(19) United States
(12) Patent Application Publication
(10) Pub. No.: US 2005/0266417 A1
(43) Pub. Date: Dec. 1, 2005

(54) Methods for identifying target nucleic acid molecules

(76) Inventors: Francis Barany, New York, NY (US); Daniel Turner, New York, NY (US); Maneesh Pingle, New York, NY (US); Hanna Pincas, New York, NY (US)

Correspondence Address: Michael L. Goldman;Nixon Peabody LLP, Clinton Square, P.O. Box 31051, Rochester, NY, 14603-1051, US

(21) Appl. No.: 10/939,294

(22) Filed: Sep. 10, 2004

Related U.S. Application Data
(60) Provisional application No. 60/502,731, filed on Sep. 12, 2003.

(51) Int. Cl. C12Q 1/68

(52) U.S. Cl. 435/6

(57) ABSTRACT

The present invention relates to methods for identifying target nucleic acid molecules differing by one or more single-base changes, insertions, deletions, or translocations; and identifying one or more target mRNA molecules differing by one or more splice site variations in a plurality of mRNA molecules. Also disclosed is a method of generating a linearly amplified representation of a whole genome. Other aspects of the present invention relate to labeled detection oligonucleotide probes and translational oligonucleotide probes as well as to methods of designing such probes.

This application claims the benefit of U.S. Provisional Patent Application Ser. No. 60/502,731, filed Sep. 12, 2003.

The subject matter of this application was made with support from the United States Government under the National Institutes of Health, Grant No. P01 CA65930-06. The U.S. Government may have certain rights.

### FIELD OF THE INVENTION

The present invention relates to methods for identifying and quantifying target nucleic acid molecules differing by single-base changes, insertions, deletions, copy number changes, or translocations, or target mRNA molecules differing by one or more splice site variations.

### BACKGROUND OF THE INVENTION

The need for large-scale multiplexed analysis of polymorphic loci and gene copy number status across the genome has intensified in recent years, especially in cancer biology. Despite the successes of microarrays for expression profiling, with notable exceptions, they have been unsuccessful in predicting patient outcomes or helping to identify new candidate cancer genes. For example, important DNA alterations in known cancer genes (mutations, methylation, gene copy number, allele imbalance, LOH) are frequently not detected by gene expression arrays. To date, DNA changes in individual genes, loss of heterozygosity (LOH), or microsatellite instability (MSI) have been the strongest predictors of prognosis in colon cancer.

Genes that have alterations at both the DNA and RNA level are more likely to lead to tumor development. There is thus an urgent need to profile DNA and RNA changes on the same tumors. Moreover, sophisticated mathematical mining of RNA expression profiles for new candidates is likely to require stratification of tumors by DNA alterations in known cancer genes.

Cancers arise from the accumulation of inherited and/or sporadic mutations at the DNA level within cell cycle, DNA repair, and growth signaling genes. Knowledge of these molecular changes can influence patient management. For instance, members of certain ethnic groups have a higher risk of carrying single nucleotide polymorphisms (SNPs) in cancer genes such as BRCA1, BRCA2 or APC. These SNPs confer an increased risk of developing breast, ovarian, prostate or colon cancers and carriers would benefit from increased vigilance in testing (Laken et al., “Genotyping by Mass Spectrometric Analysis of Short DNA Fragments,” Nature Biotechnology 16:1352-6 (1998); Abeliovich et al., “The Founder Mutations 185delAG and 5382insC in BRCA1 and 6174delT in BRCA2 Appear in 60% of Ovarian Cancer and 30% of Early-Onset Breast Cancer Patients among Ashkenazi Women,” Am. J. Human Gen. 60:505-514 (1997); Beller et al., “High Frequency of BRCA1 and BRCA2 Germline Mutations in Ashkenazi Jewish Ovarian Cancer Patients, Regardless of Family History,” Gyn. Oncol. 67:123-126 (1997); Berman et al., “A Common Mutation in BRCA2 that Predisposes to a Variety of Cancers is Found in Both Jewish Ashkenazi and Non-Jewish Individuals,” Cancer Res. 56:3409-3414 (1996); Oddoux et al., “The Carrier Frequency of the BRCA2 6174delT Mutation Among Ashkenazi Jewish Individuals is Approximately 1%,” Nature Genetics 14:188-190 (1996); Roa et al., “Ashkenazi Jewish Population Frequencies for Common Mutations in BRCA1 and BRCA2,” Nature Genetics 14:185-187 (1996); Struewing et al., “The Carrier Frequency of the BRCA1 185delAG Mutation is Approximately 1 Percent in Ashkenazi Jewish Individuals,” Nature Genetics 11:198-200 (1995); Struewing et al., “The Risk of Cancer Associated with Specific Mutations of BRCA1 and BRCA2 among Ashkenazi Jews,” New Engl. J. Med. 336:1401-8 (1997)). Sporadic mutations, such as those in the p53 gene, influence both clinical outcome and response to therapy (Broll et al., “Expression of p53 and mdm2 mRNA and Protein in Colorectal Carcinomas,” Eur. J. Cancer 35:1083-1088 (1999); Bunz et al., “Disruption of p53 in Human Cancer Cells Alters the Responses to Therapeutic Agents,” Clin. Invest. 104:263-269 (1999); Dameron et al., “Control of Angiogenesis in Fibroblasts by p53 Regulation of Thrombospondin-1,” Science 265:1582-1584 (1995); Heide et al., “The Status of p53 in the Metastatic Progression of Colorectal Cancer. Eur. J. Cancer 33:1314-1322 (1997); Prives et al., “The p53 Pathway,” J. Path. 187:112-126 (1999); Tortola et al., “p53 and K-ras Gene Mutations Correlate With Tumor Aggressiveness But Are Not of Routine Prognostic Value in Colorectal Cancer,” J. Clin. Oncol. 17:375 (1999); Zou et al., “p53 Regulates the Expression of the Tumor Suppressor Gene Maspin,” J. of Biol. Chem. 275:6051-6054 (2000)). The precise nature of the p53 mutation, therefore, may alter treatment protocols and other clinical considerations (Aurelio et al., “Germ-line-derived Hinge Domain p53 Mutants Have Lost Apoptotic But Not Cell Cycle Arrest Fnctions,” Cancer Res. 58:2190-2195 (1998); Aurelio et al., “p53 Mutants have Selective Dominant-Negative Effects on Apoptosis but not Growth Arrest,” Molec. Cell Biol. 20:770-778 (2000); Foster et al., “Pharmacological Rescue of Mutant p53 Conformation and Function,” Science 286:2507-2510 (1999); Wang et al., “Induced p53 Expression in Lung Cancer Cell Line Promotes Cell Senescence and Differentially Modifies the Cytotoxicity of Anti-Cancer Drugs,” Oncogene 17:1923-1930 (1998); Webley et al., “Effect of Mutation and Conformation on the Function of p53 in Colorectal Cancer,” J. Path. 191:361-367 (2000)).

In addition to the inherited and sporadic mutations, many tumors exhibit aneuploidy and chromosomal instability (CIN) in which the diploid structure of the genome is corrupted. Chromosomal rearrangement is common, and the genome may exhibit amplification of alleles, or conversely the loss of an allele—“loss of heterozygosity” (LOH). These changes serve to characterize the unique molecular signature of the tumor further and, consequently, also influence patient management. It is imperative, therefore, that detection techniques have the ability to accurately identify and describe these changes.

SNPs are recognized as powerful markers that may be used to help identify genes responsible for complex diseases including cancer. Fundamental concepts intrinsic to these genetic models include “linkage” and “linkage disequilibrium” (LD). Linkage refers to the tendency of two or more loci to be inherited together because of their location near to one another on the same chromosome. Tightly linked loci do not assort independently during the creation of germ cells (meiosis). This results in a state of disequilibrium, since the two-locus haplotype frequency for a pair of alleles is not equal to the product of the individual allele frequencies. This difference reflects the degree of linkage disequilibrium (Lynch et al., Genetics and Analysis of Quantitative Traits (1997).

These principles form the conceptual foundation for a whole-genome based candidate gene identification approach, often referred to as an association study. Here, geneticists attempt to correlate SNP patterns with phenotypes (e.g. normal vs. disease) to predict the locations of susceptibility genes. In principle, if a genomic region demonstrates high LD, then multiple polymorphisms within that region will be inherited together (co-segregate), thereby behaving in aggregate. In such regions, a single polymorphic site can be used as a marker for the entire set of loci. Therefore, when attempting to identify a susceptibility gene across the entire genome, regions of relatively higher LD would require fewer loci to be genotyped, because a single locus can represent a larger set (Hoh et al., “Trimming, Weighting and Grouping SNPs in Human Case-Control Association Studies,” Genome Res. 11:2115-2119 (2001)).

Association studies using LD to identify susceptibility genes have achieved some recent success. Proof of principle experiments using a high-density SNP map in genomic regions already known to contain susceptibility genes for complex diseases (including Parkinson's, Alzheimer's, psoriasis, migraine, type II diabetes, and Crohn's disease) have confirmed known genes or identified new ones (Rioux et al., “Genetic Variation in the 5q31 Cytokine Gene Cluster Confers Susceptibility to Crohn Disease,” Nature Genetics 29:223-228 (2001); Hewett et al., “Identification of a Psoriasis Susceptibility Candidate Gene by Linkage Disequilibrium Mapping with a Localized Single Nucleotide Polymorphism Map,” Genomics 79:305-314 (2002); Martin et al., “SNPing Away at Complex Diseases: Analysis of Single-Nucleotide Polymorphisms Around APOE in Alzheimer Disease,” Am. J. Human Genet. 67:383-394 (2000); Martin et al., “Association of Single-Nucleotide Polymorphisms of the tau gene with Late-Onset Parkinson Disease,” J. Am. Med. Assoc. 286:2245-2250 (2001); McCarthy et al., “Single Nucleotide Polymorphism Alleles in the Insulin Receptor Gene are Associated with Typical Migraine,” Genomics 78:135-149 (2001); Horikawa et al., “Genetic Variation in the Gene Encoding Calpain-10 is Associated with Type 2 Diabetes Mellitus,” Nature Genetics 26:135-137 (2000)).

These successful association studies are “candidate gene” approaches, where the location of the susceptibility gene is either already known or suspected. A more global approach attempts to identify the location of unknown susceptibility genes across the whole genome, without bias to initial candidate locations. This experimental design is based on a two step LD methodology (Roses, A. D., “Pharmacogenetics and the Practice of Medicine,” Nature 405:857-865 (2000)). In the first step, a low-density SNP map (i.e. “picket fence”) of the whole genome is used to identify a region(s) containing the potential susceptibility gene(s). Once the region is identified, a high-density SNP map is used to home in on the susceptibility gene within the region. The success of this approach greatly depends upon the degree of LD across the human genome, since this will determine the number and location of SNPs required to make meaningful predictions (estimates range from 10,000-30,000 for select ethnic populations, 100,000-200,000 for outbred populations, to as many as 1,000,000 SNPs). Thus, there is an urgent need to accurately score thousands of SNPs in a cost effective manner (Kruglyak, L., “Prospects for Whole-Genome Linkage Disequilibrium Mapping of Common Disease Genes,” Nature Genetics 22(2):139-44 (1999); Eaves et al., “The Genetically Isolated Populations of Finland and Sardinia may not be a Panacea for Linkage Disequilibrium Mapping of Common Disease Genes,” Nature Genetics 25(3):320-3 (2000); Taillon-Miller et al., “Juxtaposed Regions of Extensive and Minimal Linkage Disequilibrium in Human Xq25 and Xq28,” Nature Genetics 25:324-8 (2000); Collins et al., “Genetic Epidemiology of Single-Nucleotide Polymorphisms,” Proc. Nat'l Acad. Sci. USA 96:15173-7 (1999); Roses, A., “Pharmacogenetics Place in Modern Medical Science and Practice,” Life Sciences 70:1471-1480 (2002)).

Returning to the problem of tumor aneuploidy, some LOH and gene amplification events reflect the presence of tumor suppressor genes and oncogenes respectively. To track such changes accurately throughout the genome at a low resolution of 1 MB regions would require scoring 3,000 informative SNPs for each sample. Since not every SNP in a given individual is informative, a minimum of some 10,000 SNPs would need to be interrogated. Since solid tumors contain a mixture of both tumor and non-tumor (stromal) cells, analysis of copy number changes in tumor cells is complicated by the presence of the extra contaminating stromal cells with diploid copy numbers. While preparations of “pure” tumor cells may be prepared by laser capture microscopy (LCM), this limits the number of tumor cells for analysis to a few hundred. There is thus an urgent need to accurately quantify allele imbalance in thousands of SNPs in tumor cells, both in the presence of stromal cells and when starting with limiting amounts of tumor cells.

Thus, to recapitulate, there are two major uses of SNPs in genetics and tumor biology that require high-throughput and quantitative SNP scoring:
1. Use of SNPs as markers of complex disease.
2. Use of SNPs as markers of chromosome regions, to determine gains and losses in tumors.

The total number of SNPs that can be scored is limited by the amount of sample available. When sample DNA is in short supply, it can be amplified by polymerase chain reaction (PCR). However, the PCR step can generate cross-contamination artifacts, affecting the overall specificity and sensitivity, and can yield variable multiplexing results, limiting the throughput of the assay. Since the majority of methodologies currently available use PCR amplification, development of an alternative method for the high throughput genotyping of low quantity DNA samples (such as from tumor biopsies) would be desirable.

Current gene and mutation detection techniques fall into three general strategies; (i) target amplification (i.e. PCR, whole genome amplification (WGA), strand displacement assay (SDA)), (ii) probe amplification (ligase chain reaction (LCR), ligase detection reaction (LDR), rolling circle amplification (RCA)), and (iii) signal amplification (3D dendrimer labeling systems, enzymatic cascade reporters). Combining two orthogonal techniques, such as PCR with LDR can significantly improve signal to noise.

Multiple mutations or polymorphisms on multiple genes can be detected using PCR/PCR/LDR/Universal Array, which has the advantage of amplifying many regions together, before the LDR is performed (See Belgrader et al., “A Multiplex PCR-Ligase Detection Reaction Assay for Human Identity Testing,” Genome Sci. Technol. 1:77-87 (1996); and Favis et al., “Multiplex PCR/PCR/LDR Detection of BRCA1 and BRCA2 Small Insertions and Deletions Using a Universal DNA Array,” Nature Biotechnology 18:561-564 (2000)). Gene-specific PCR primers (all of which share a universal sequence on their 5′ ends) are used at low concentration for an initial simultaneous amplification of multiple regions using a limited number of PCR cycles. All primary fragments are then amplified with additional PCR cycles using the universal primer. Adjacent ligation probes, one containing a fluorescent label the other a complementary zip-code sequence, are covalently linked by ligase when there is perfect complementarity at the junction. Finally, ligation products are captured on a universal array containing the zip-code sequences. This approach has been validated for detecting K-ras and p53 gene mutations in non-microdissected tumors as well as stool, demonstrating the ability to find mutations despite a large quantity of background normal sequence. However, the method is not quantitative, and, moreover, PCR primers occasionally need to be redesigned to ensure that the different exons amplify together.

An alternative approach is to begin with an LDR step where discriminating LDR probes, encoded by a zip-code sequence, are used in conjunction with a common set of universal PCR primers. Amplification products are then rendered single-stranded and are identified by capturing at the zip-code sequence on a universal array. The advantage of this approach is the ability to multiplex about 100 probe sets together, but as with the previous example, the method is not quantitative. Furthermore, there are problems of some alleles “dropping out” during the PCR step. Even though these approaches are the most robust for multiplexing gene amplifications to date, they may not detect all potential biothreats in clinical samples. Since the dropout problem is a consequence of the PCR step, performing the LDR without a PCR step may allow quantitative scoring of hundreds to thousands of genes or SNPs.

There are certain practical limits to performing a straight multiplexed LDR reaction querying for hundreds of genes or several thousand SNPs, and then attempting to capture the LDR products on a zip-code array. A certain minimum number of molecules is needed for any hybridization-based assay to achieve efficient capture in a reasonable period of time, as required for a clinical test. For a hybridization event to take place successfully, the two DNA molecules need to encounter one another in the correct orientation. The problem is exacerbated when one of the molecules is tethered to a large support, as this limits the degree of freedom. Thermodynamically, an array will eventually capture a few molecules of complementary target DNA, but kinetically, it may take days or even weeks. The reason why PCR works is because initially there is a million-fold excess of primer over target DNA, so the hybridizations take place very rapidly.

One approach to circumvent arrays is to encode a tiny bar with gold and silver (known as molecular bar coding). Another approach is to put highly fluorescent nanocrystals, known as quantum dots (Q-dots) into beads (Han et al., “Quantum-Dot-Tagged Microbeads for Multiplexed Optical Coding of Biomolecules,” Nature Biotechnology, 19:631-635 (2001)).

Thus, the need remains for a rapid single assay to identify or quantify accurately dozens to hundreds of single base differences, copy number changes, insertions, deletions, loss of heterozygosity, or translocations in a polynucleotide sample.

The present invention is directed toward overcoming these deficiencies in the art.

### SUMMARY OF THE INVENTION

The present invention relates to a method for identifying one or more target nucleic acid molecules differing by one or more single-base changes, insertions, deletions, or translocations. This method involves providing a test sample potentially containing one or more target nucleic acid molecules differing by one or more single-base changes, insertions, deletions, or translocations. Also provided is one or more primary oligonucleotide probe sets, each set characterized by (a) a first oligonucleotide probe, having a target-specific portion and (b) a second oligonucleotide probe, having a target-specific portion, where the oligonucleotide probes in a particular set are suitable for ligation together when hybridized on a corresponding target nucleic acid molecule, but have a mismatch which interferes with such ligation when hybridized to any other nucleic acid molecule present in the sample, and where one or both oligonucleotide probes in the set contain one or more detection oligonucleotide probe-specific portions or their complements such that each probe set contains a unique set of one or more detection oligonucleotide probe-specific portions or their complements. The sample, the one or more primary oligonucleotide probe sets, and a ligase are blended to form a primary ligase detection reaction mixture. The primary ligase detection reaction mixture is subjected to one or more ligase detection reaction cycles. These cycles each include a denaturation treatment, where any hybridized oligonucleotides are separated from the target nucleic acid molecules, and a hybridization treatment, where the primary oligonucleotide probe sets hybridize in a base-specific manner to their respective target nucleic acid molecules, if present in the sample, and ligate to one another to form a primary ligation product containing the target-specific portions and one or more detection oligonucleotide probe-specific portions or their complements. The primary ligation product for each of the primary oligonucleotide probe sets are distinguishable from other nucleic acid molecules in the primary ligase detection reaction mixture by a unique set of one or more detection oligonucleotide probe-specific portions or their complements. The primary oligonucleotide probe sets may hybridize to nucleic acid molecules in the sample other than their respective target nucleic acid molecules but do not ligate together due to a presence of one or more mismatches and individually separate during the denaturation treatment. Detection oligonucleotide probes which bind to the complementary detection oligonucleotide probe-specific portion of the captured primary ligation product or complements thereof are provided, where each detection oligonucleotide probe has a reporter label, thereby providing each primary ligation product with a unique detectable encryption code. The primary ligation products and the detection oligonucleotide probes are contacted under conditions effective to permit hybridization of the detection oligonucleotide probes to the primary ligation products so that a labeled primary ligation product is formed. The reporter label(s) on the primary ligation product are then detected, thereby indicating the presence of one or more target nucleic acid molecules in the sample. Nucleic acid molecules differing by one or more single-base changes, insertions, deletions, or translocations are discriminated from one another during the one or more ligase detection reaction cycles and the discriminated nucleic acid molecules are detected as a result of each differently labeled, primary ligation product having a unique encryption code with a different pattern of detectable emission spectra.

Another embodiment of the present invention relates to a method for identifying one or more target nucleic acid molecules differing by one or more single-base changes, insertions, deletions, or translocations. This method involves providing a test sample potentially containing one or more target nucleic acid molecules. One or more primary oligonucleotide probe sets, each set characterized by (a) a first oligonucleotide probe, having a target-specific portion and a 5′ upstream portion containing a translational oligonucleotide portion and (b) a second oligonucleotide probe, having a target-specific portion, and a 3′ downstream primer-specific portion are provided. The oligonucleotide probes in a particular primary oligonucleotide probe set are suitable for ligation together when hybridized to a corresponding target nucleic acid molecule, but have a mismatch which interferes with this ligation when hybridized to any other nucleic acid molecule present in the test sample. The test sample, the one or more primary oligonucleotide probe sets, and a ligase are blended to form a primary ligase detection reaction mixture. The primary ligase detection reaction mixture is subjected to one or more ligase detection reaction cycles. These cycles each include a denaturation treatment, where any hybridized oligonucleotide probes are separated from the target nucleic acid molecules, and a hybridization treatment, where the primary oligonucleotide probe sets hybridize in a base-specific manner to their respective target nucleic acid molecules, if present in the test sample, and ligate to one another to form a primary ligation product containing (a) the 5′ upstream translational oligonucleotide portion, (b) the target-specific portions, and (c) the 3′ downstream primer-specific portion. The primary ligation product for each primary oligonucleotide probe set is distinguishable from other nucleic acids in the ligase detection reaction mixture, and the primary oligonucleotide probe sets may hybridize to nucleic acid molecules in the test sample other than their respective target nucleic acid molecules but do not ligate together due to a presence of one or more mismatches and individually separate during the denaturation treatment. Also provided in this aspect of the present invention is a downstream primer complementary to the 3′ downstream primer-specific portion of the primary ligation product. The primary ligation product is blended with the downstream primer and a polymerase to form a polymerase chain reaction mixture. The polymerase chain reaction mixture is subjected to one or more polymerase chain reaction cycles comprising a denaturation treatment, where hybridized nucleic acid molecules are separated, a hybridization treatment, where the primer hybridizes to its complementary 3′ downstream primer-specific portion of the primary ligation product, and an extension treatment, where the hybridized primers are extended to form extension products complementary to the primary ligation product. The extension product is captured on one or more solid supports, so that the extension product may be individually distinguished. Also provided are one or more secondary oligonucleotide probe sets, each set characterized by (a) a first oligonucleotide probe, having a translational oligonucleotide portion and a 5′ upstream portion complementary to one or more detection oligonucleotide probe-specific portions and (b) a second oligonucleotide probe, having a target portion, and a 3′ downstream portion complementary to one or more detection oligonucleotide probe-specific portions. The oligonucleotide probes in a particular secondary oligonucleotide probe set are suitable for ligation together when hybridized to a corresponding captured primary extension product, but have a mismatch which interferes with this ligation when hybridized to any other nucleic acid molecule. The captured extension product, the one or more secondary oligonucleotide probe sets, and the ligase are blended to form a second ligase detection reaction mixture. The second ligase detection reaction mixture is subjected to one ligase detection reaction cycle. This cycle comprises a denaturation treatment, where any hybridized oligonucleotides are separated from the captured extension product, and a hybridization treatment, where the secondary oligonucleotide probe sets hybridize in a base-specific manner to their respective captured extension products, if present, and ligate to one another to form a secondary ligation product containing (a) the 5′ upstream portion comprising one or more detection oligonucleotide probe-specific portions, (b) the upstream translational oligonucleotide portion connected to the target portion, and (c) the 3′ downstream portion comprising one or more detection oligonucleotide probe-specific portions. The secondary ligation product for each secondary oligonucleotide probe set are distinguishable from other nucleic acids in the second ligase detection reaction mixture. The one or more secondary oligonucleotide probe sets may hybridize to nucleic acid molecules in the sample other than their respective captured extension products but do not ligate together due to the presence of one or more mismatches and individually separate during the denaturation treatment by heating to above the temperature at which each translational oligonucleotide portion melts, but below a temperature at which each secondary ligation product melts. As a result, each secondary ligation product remains hybridized to the captured extension product as a complex. Also provided are detection oligonucleotide probes which bind to the detection oligonucleotide probe-specific portions of the complex, where each detection oligonucleotide probe has a reporter label, thereby providing each complex containing a secondary ligation product with a unique detectable encryption code. The complex and the detection oligonucleotide probes are contacted under conditions effective to permit hybridization of the detection oligonucleotide probes to the complex so that a labeled complex is formed. The reporter label(s) on the complex are then detected, thereby indicating the presence of one or more target nucleic acid molecule in the test sample. As a result, nucleic acid molecules differing by one or more single-base changes, insertions, deletions, or translocations are discriminated from one another during the primary and secondary ligase detection reactions and the discriminated nucleic acid molecules are detected as a result of each different labeled complex having a unique encryption code with a different pattern of detectable emission spectra.

Another aspect of the present invention is directed to a method for identifying one or more target nucleic acid molecules, differing by one or more single-base changes, insertions, deletions, or translocations, in a plurality of nucleic acid molecules or identifying one or more target mRNA molecules differing by one or more splice site variations in a plurality of mRNA molecules. This method involves providing a test sample potentially containing one or more target nucleic acid molecules differing by one or more single-base changes, insertions, deletions, or translocations, in a plurality of nucleic acid molecules or one or more target mRNA molecules differing by one or more splice site variations in a plurality of mRNA molecules. One or more primary oligonucleotide probe sets are provided. Each set is characterized by (a) a first oligonucleotide probe, having one or more detection oligonucleotide probe-specific portions or their complements and a target-specific portion and (b) a second oligonucleotide probe, having a target-specific portion and an addressable array-specific portion or its complement. The oligonucleotide probes in a particular set are suitable for ligation together when hybridized to a corresponding target nucleic acid molecule or target mRNA molecule, but have a mismatch which interferes with this ligation when hybridized to any other nucleic acid molecule or mRNA molecule present in the sample, and such that each probe set contains a unique combination of detection oligonucleotide probe-specific portions and addressable array-specific portions or their complements. The sample, the one or more oligonucleotide probe sets, and a ligase are blended to form a primary ligase detection reaction mixture. The primary ligase detection reaction mixture is then subjected to one or more primary ligase detection reaction cycles. These cycles include a denaturation treatment, where any hybridized oligonucleotides are separated from the target nucleic acid molecules or target mRNA molecules, and a hybridization treatment, where the one or more oligonucleotide probe sets hybridize in a base-specific manner to their respective target nucleic acid molecules or target mRNA molecules, if present in the sample, and ligate to one another to form a primary ligation product containing (a) the one or more detection oligonucleotide probe-specific portions or their complements, (b) the target-specific portions, and (c) the addressable array-specific portion or its complement. The primary ligation product for each one or more oligonucleotide probe set is distinguished from other nucleic acid molecules or mRNA molecules in the primary ligase detection reaction mixture by virtue of their containing a unique combination of detection oligonucleotide probe-specific portions and addressable array-specific portions or their complements. The primary oligonucleotide probe sets may hybridize to nucleic acid molecules or mRNA molecules in the sample other than their respective target nucleic acid molecules or target mRNA molecules but do not ligate together due to the presence of one or more mismatches and individually separate during the denaturation treatment. Also provided is a solid support with capture oligonucleotide probes immobilized at different sites, where the capture oligonucleotide probes have nucleotide sequences complementary to the addressable array-specific portions or their complements. The primary ligation products, copies of primary ligation products, or complements thereof are contacted with the solid support under conditions effective to hybridize the primary ligation products, copies of primary ligation products, or complements thereof to the capture oligonucleotide probes in a base-specific manner. As a result, the primary ligation products, copies of primary ligation products, or complements thereof are captured on the solid support at the site with the complementary capture oligonucleotide. Detection oligonucleotide probes are provided which bind to the detection oligonucleotide probe-specific portions of the captured primary ligation products, copies of primary ligation products, or complements thereof. Each detection oligonucleotide probe has a reporter label, so that each of the captured primary ligation products, copies of primary ligation products, or complements thereof have a unique detectable encryption code. The captured primary ligation products, copies of primary ligation products, or complements thereof are contacted with the detection oligonucleotide probes under conditions effective to permit hybridization of the detection oligonucleotide probes to the captured primary ligation products, copies of primary ligation products, or complements thereof so that labeled, captured primary ligation products, copies of primary ligation products, or complements thereof are formed. The reporter labels on the labeled, captured primary ligation products, copies of primary ligation products, or complements thereof are detected, thereby indicating the presence of one or more target nucleic acid molecules or target mRNA molecules in the sample. As a result, target nucleic acid molecules differing by one or more single-base changes, insertions, deletions, or translocations in a plurality of nucleic acid molecules or target mRNA molecules differing by one or more splice site variations in a plurality of mRNA molecules are discriminated from one another during the primary ligase detection reaction. The discriminated molecules are detected as a result of different labeled ligation products having encryption codes with a different pattern of detectable emission spectra, at different sites on the solid support.

Another embodiment of the present invention relates to a method for identifying one or more target nucleic acid molecules, differing by one or more single-base changes, insertions, deletions, or translocations, in a plurality of nucleic acid molecules or identifying one or more target mRNA molecules differing by one or more splice site variations in a plurality of mRNA molecules. This involves providing a sample potentially containing one or more target nucleic acid molecules differing by one or more single-base changes, insertions, deletions, or translocations or one or more target mRNA molecules differing by one or more splice site variations. The target nucleic acid molecules or target mRNA molecules in the sample are captured on one or a plurality of solid supports, so that they may be individually distinguished. One or more primary oligonucleotide probe sets are provided, each set characterized by (a) a first oligonucleotide probe, having a target-specific portion and a 5′ upstream portion complementary to one or more detection oligonucleotide probes and (b) a second oligonucleotide probe, having a target-specific portion and a 3′ downstream portion complementary to one or more detection oligonucleotide probes. The oligonucleotide probes in a particular set are suitable for ligation together when hybridized to one another on a corresponding target nucleic molecule, but have a mismatch which interferes with this ligation when hybridized to any other nucleic molecule present in the sample. Each probe set contains a unique set of 5′ upstream and 3′ downstream portions. The sample, the one or more primary oligonucleotide probe sets, and the ligase are blended to form a primary ligase detection reaction mixture. The primary ligase detection reaction mixture is subjected to one or more ligase detection reaction cycles. These cycles include a denaturation treatment, where any hybridized oligonucleotides are separated from the target nucleic acid molecule or target mRNA molecule, and a hybridization treatment, where the oligonucleotide probe sets hybridize in a base-specific manner to their respective target nucleic acid molecules or target mRNA molecule, if present in the sample, and ligate to one another to form a primary ligation product. The primary ligation product contains (a) the 5′ upstream portion, (b) the target-specific portions, and (c) the 3′ downstream portion with the primary ligation product remaining bound to the captured target nucleic acid molecule or target mRNA molecule. The primary ligation product for each primary oligonucleotide probe set is distinguishable from other nucleic acids in the primary ligase detection reaction mixture by virtue of its containing a unique set of 5′ upstream portion and 3′ downstream portion. The oligonucleotide probe sets may hybridize to nucleic acid molecules in the sample other than their respective target nucleic acid molecule or target mRNA sequences but do not ligate together due to the presence of one or more mismatches and individually separate during the denaturation treatment. Also provided are detection oligonucleotide probes which bind to the 5′ upstream and 3′ downstream portions of the ligation products. Each detection oligonucleotide probe has a reporter label, so that each labeled primary ligation product has a unique detectable encryption code. The primary ligation products and the detection oligonucleotide probes are contacted under conditions effective to permit hybridization of the detection oligonucleotide probes to the primary ligation products so that a labeled captured primary ligation product is formed. The reporter labels on the primary ligation products are detected. As a result, the presence of one or more target nucleic acid molecules differing by one or more single base changes, insertions, deletions, or translocations or one or more target mRNA molecules differing by one or more splice site variations are discriminated from one another during the ligase detection reaction and the discriminated target nucleic acid molecules or the target mRNA molecules are detected due to each different reporter label having a unique encryption code with a different pattern of detectable emission spectra.

Another aspect of the present is directed to a method for identifying one or more target nucleic acid molecules differing by one or more single-base changes, insertions, deletions, or translocations in a plurality of nucleic acid molecules. This method involves providing a test sample potentially containing one or more target nucleic acid molecules. Also provided are one or more primary oligonucleotide probe sets, each set characterized by (a) a first oligonucleotide probe, having a target-specific portion and a 5′ upstream portion containing one or more translational oligonucleotide probes or their complements, and (b) a second oligonucleotide probe, having a target-specific portion. The oligonucleotide probes in a particular primary oligonucleotide set are suitable for ligation together when hybridized to a corresponding target nucleic acid molecule, but have a mismatch which interferes with this ligation when hybridized to any other nucleic acid molecule present in the sample. Each probe set contains a unique combination of translational oligonucleotide probes or their complements. The sample, the one or more primary oligonucleotide probe sets, and the ligase are blended to form a primary ligase detection reaction mixture. The primary ligase detection reaction mixture is subjected to one or more ligase detection reaction cycles. These cycles include a denaturation treatment, where any hybridized oligonucleotides are separated from the target nucleic acid molecules, and a hybridization treatment, where the oligonucleotide probes or the primary oligonucleotide probe sets hybridize in a base-specific manner to their respective target nucleic acid molecules, if present in the sample, and ligate to one another to form a primary ligation product. The primary ligation product contains (a) the translational oligonucleotide-specific portion or their complements and (b) the target-specific portions. The ligation product for each primary oligonculeotide probe set is distinguishable from other nucleic acids in the primary ligase detection reaction mixture by virtue of its containing a unique combination of translational oligonucleotide portions or their complements. The primary oligonucleotide probe sets may hybridize to nucleic acid molecules in the sample other than their respective target nucleic acid molecules but do not ligate together due to the presence of one or more mismatches and individually separate during the denaturation treatment. Also provided are one or more secondary oligonucleotide probe sets, each set characterized by (a) a first oligonucleotide probe, having a translational oligonucleotide-specific portion and a 5′ upstream portion complementary to one or more detection oligonucleotide probe sequences and (b) a second oligonucleotide probe, having a translational oligonucleotide-specific portion, and a 3′ downstream portion complementary to one or more detection oligonucleotide probe sequences. The oligonucleotide probes in a particular secondary oligonucleotide probe set are suitable for ligation together when hybridized to a corresponding complement of a primary ligation product, but have a mismatch which interferes with this ligation when hybridized to any other nucleic acid molecule present in the sample. As a result, each secondary oligonucleotide probe set contains a unique set of 5′ upstream and 3′ downstream portions. The primary ligation products, the plurality of secondary oligonucleotide probe sets, and the ligase are blended to form a secondary ligase detection reaction mixture. The secondary ligase detection reaction mixture is subjected to one ligase detection reaction cycle. Each cycle includes a denaturation treatment, where any hybridized oligonucleotide probes are separated from nucleic acid molecules to which they are hybridized, and a hybridization treatment, where the secondary oligonucleotide probe sets hybridize in a base-specific manner to their corresponding primary ligation products, if present, and ligate to one another to form a secondary ligation product containing (a) the 5′ upstream portion complementary to one or more distinct oligonucleotide probe sequences, (b) the upstream translational oligonucleotide-specific portion, (c) the downstream translational oligonucleotide-specific portion, and (d) the 3′ downstream portion complementary to one or more distinct oligonucleotide probe sequences. The secondary oligonucleotide probe sets may hybridize to nucleic acid molecules other than their respective primary ligation products but do not ligate together due to the presence of one or more mismatches and individually separate during the denaturation treatment. Also provided are detection oligonucleotide probes which bind to the 5′ upstream portion and the 3′ downstream portion, where each detection oligonucleotide probe has a reporter label, so that each of the products has a unique detectable encryption code. The secondary ligation products and the detection oligonucleotide probes are contacted under conditions effective to permit hybridization of the detection oligonucleotide probes to the ligation products so that labeled, secondary ligation products are formed. The reporter labels on the labeled, secondary ligation products are detected. The presence of one or more target nucleic acid molecules in the sample is indicated, where sequences differing by one or more single-base changes, insertions, deletions, or translocations are discriminated from one another during the primary ligase detection reaction. The discriminated sequences are detected as a result of each different labeled secondary ligation product having a unique encryption code with a different pattern of detectable emission spectra.

Another aspect of the present invention relates to a method of generating a linearly amplified representation of a whole genome. This involves providing genomic DNA molecules and subjecting the genomic DNA molecules to enzymatic digestion with a first restriction endonuclease to produce a degenerate oligonucleotide fragment with a degenerate overhang. A hairpin linker is provided which contains an overhang complementary to the degenerate overhang and a modification complementing the 5′ end of the degenerate oligonucleotide fragment within a second restriction site. The modification blocks restriction endonuclease cleavage on the 5′ side, but not the 3′ side, of the degenerate oligonucleotide fragment. The enzymatic digestion step is carried out in the presence of the hairpin linker, a ligase, and a first restriction endonuclease, under conditions effective to permit cleavage of genomic DNA molecules and ligation of the hairpin linker onto ends of the degenerate oligonucleotide fragments. Unligated linkers are then removed. Also provided are a second restriction endonuclease and a processive DNA polymerase with strand-displacement activity. The enzymatically digested genomic DNA molecules, the second restriction endonuclease, and the polymerase are blended to form a representational genome amplification mixture. The representational genome amplification mixture is incubated under conditions effective to permit the second restriction endonuclease to nick the hairpin DNA on its unmodified strand and the polymerase to extend the degenerate oligonucleotide fragments at their free 3′ ends. As the polymerase extends and displaces the pre-existing strand, it reproduces the second restriction site allowing for repeated nicking/polymerase extension and linear amplification of a representation of the whole genome.

Another aspect of the present relates to a method of designing a plurality of labeled detection oligonucleotide probes for use in combinations of one to four or more probes to identify or quantify complementary sequences which will hybridize with little mismatch. The plurality labeled oligonucleotide probes have melting temperatures within a narrow range. This method involves providing a first set of a plurality of tetramers of four nucleotides linked together, where (1) each tetramer within the set differs from all other tetramers in the set by at least two nucleotide bases, (2) no two tetramers within a set are complementary to one another, (3) no tetramers within a set are palindromic or dinucleotide repeats, and (4) no tetramer within a set has less than one or more than three G and C nucleotides. Groups of 2 to 4 tetramers from the first set are linked together to form a collection of multimer units. All multimer units formed from the same tetramer and all multimer units having a melting temperature in ° C. of less than 6 times the number of tetramers forming a multimer unit are removed from the collection of multimer units, to form a modified collection of multimer units. A second collection of multimer units are selected from the modified collection of multimer units such that no consecutive tetramer pair is used twice. 1 or 2 tetramers are added to either or both ends of the second collection of multimers to generate a new set of modified multimer units with higher melting temperatures, so that no consecutive tetramer pair is used twice. The new set of modified multimer units are arranged in a list in order of melting temperature. Units having a melting temperature in ° C. of less than 12 times the number of tetramers and more than 18 times the number of tetramers are removed from the set of modified multimer units to form a further collection of multimer units. Reporter labels are linked to the further collection of multimer units, to form labeled detection oligonucleotide probes.

The present invention also relates to a method of designing a plurality of translational oligonucleotides for attachment to target-specific oligonucleotide probes to identify or quantify complementary sequences which will hybridize with little mismatch, where the plural translating oligonucleotide sequences have melting temperatures within a narrow range. This involves providing a first set of a plurality of tetramers of four nucleotides linked together, where (1) each tetramer within the set differs from all other tetramers in the set by at least two nucleotide bases, (2) no two tetramers within a set are complementary to one another, (3) no tetramers within a set are palindromic or dinucleotide repeats, and (4) no tetramer within a set has less than one or more than three G and C nucleotides. Groups of 2 to 4 tetramers from the first set are linked together to form a collection of multimer units. All multimer units formed from the same tetramer and all multimer units having a melting temperature in ° C. of less than 3 times the number of tetramers forming a multimer unit are removed from the collection of multimer units to form a modified collection of multimer units. The modified collection of multimer units are arranged in a list in order of melting temperature. The order of the modified collection of multimer units is randomized in 0.1° C. increments of melting temperature. Alternating multimer units in the list are divided into first and second subcollections, each arranged in order of melting temperature. The order of the second subcollection is inverted. In order, the first subcollection of multimer units is linked to the inverted second subcollection of multimer units in order to form a collection of double multimer units. The collection of double multimer units is arranged in a list in order of melting temperature. Those units having a melting temperature in ° C. of less than 12 times the number of tetramers and more than 18 times the number of tetramers are removed from the ordered collection of double multimer units to form a modified collection of multimer units. The double multimer units are linked to a target-specific oligonucleotide probe.

The present invention also relates to a collection of labeled detection oligonucleotide probes. These probes include a collection of detection oligonucleotide probes to which complementary oligonucleotide probes will hybridize, within a narrow temperature range of greater than 24° C. with little mismatch. The oligonucleotide probes are formed from sets of tetramers where (1) each tetramer within the set differs from all other tetramers in the set by at least two nucleotide bases, (2) no two tetramers within a set are complementary to one another, (3) no tetramers within a set are palindromic or dinucleotide repeats, and (4) no tetramer within a set has less than one or more than three G and C nucleotides. The collection of oligonucleotide probes has oligonucleotides having a melting temperature in ° C. less than 12 times the number of tetramers and more than 18 times the number of tetramers. This aspect of the present invention also includes reporter labels linked to each of the oligonucleotide probes in the collection.

The present invention also relates to a collection of fusion oligonucleotide probes. These probes includes a collection of translational oligonucleotide probes to which complementary oligonucleotide probes will hybridize, within a narrow temperature range of greater than 24° C. with little mismatch. The oligonucleotide probes are formed from sets of tetramers where (1) each tetramer within the set differs from all other tetramers in the set by at least two nucleotide bases, (2) no two tetramers within a set are complementary to one another, and (3) no tetramers within a set are palindromic or dinucleotide repeats, and (4) no tetramer within a set has less than one or more than three G and C nucleotides. The collection of oligonucleotide probes has oligonucleotides having a melting temperature in ° C. less than 12 times the number of tetramers and more than 18 times the number of tetramers. Target-specific oligonucleotide probes linked to each of the oligonucleotide probes in the collection.

The methods of the present invention can be used to detect a wide variety of infectious diseases, genetic diseases, and cancer and can be applied to biodefense, environmental monitoring, forensics, and the food and feed industry. The methods hold the promise of high-throughput identification and quantification of dozens to hundreds of nucleic acid sequences in a single homogneous reaction. This will assist in determining the presence of genetic diseases that arise from deletions or copy number imbalance. Furthermore, it will help with LOH and gene dosage analysis of tumor samples on a genome-wide scale. This will be invaluable for identifying genes responsible for tumor development, and consequently new targets for therapies.

The ligase detection primers are designed to contain detection probe sequences. When two ligase detection primers hybridize adjacent to each other on the target nucleic acid, they may be ligated to each other provided there is perfect complementarity at the ligation junction. The resultant ligation product may be uniquely distinguished, identified, and quantified, separately from all other potential ligation products in the reaction mix, by virtue of containing a unique set of 2, 3, 4, or more detection probe or complementary detection probe sequences. Detection probes, containing distinct reporter groups are then hybridized directly to the ligation product, the complement of the ligation product, or an amplified form of the ligation product, and the unique combination of reporter groups is subsequently detected and scored. This process serves to assemble the unique reporter signal at a molecular level.

This invention addresses problems that could not be overcome by previous approaches. Firstly, individual reporter groups, such as nanocrystal Quantum dots are small, so they may be used at high concentrations, typically between 500 and 1,000 femtomoles compared to a limit of approximately 3 femtomoles for the smallest quantum beads (100 nM diameter). Thus, the detection probes may be used at sufficiently high concentration to allow for rapid and accurate hybridization to all ligation products. Secondly, prior art methods required 1,000 different LDR or PCR products finding and correctly hybridizing to 1,000 different quantum beads or addresses on arrays or beads or solid supports. In contrast, in the present invention, the total number of reporter detection probes has been reduced to 8, 12, or 16 sequences, which need to find and correctly hybridize to 8, 12, or 16 complementary sequences. This also reduces the total amount of labeled probe present in the reaction from 200 pmol for labeling individual probes to between 4 and 16 pmol using reporter detection probes. Furthermore, it reduces the cost of manufacturing labeled LDR probes from thousands of specialty probes to 8,12, or 16 reporter detection probes. Finally, a true signal requires 2, 3, or 4 reporter detection probes on the same DNA, and thus a positive signal can be distinguished from the non-specific binding of individual reporter detection probes that generate noise.

### BRIEF DESCRIPTION OF THE DRAWINGS

FIG. 1 is a schematic diagram of a ligase detection reaction (“LDR”) process, in accordance with the present invention, where multiple oligonucleotide probes (“Q-Zip probes”) containing quantum dots (“Q-dots”) are used on a single tail to distinguish single base differences in nucleic acid molecules.

FIG. 2 is a schematic diagram of an LDR process, in accordance with the present invention, where multiple Q-Zip probes containing Q-dots on a single tail are used with microbead capture to score allele imbalance in a tumor sample compared with a normal sample.

FIG. 3 is a schematic diagram of an LDR process, in accordance with the present invention, where multiple Q-Zip portions containing Q-dots on a single tail are used with microbead capture to score gene copy number in a tumor sample compared with a normal sample.

FIG. 4 is a schematic diagram of an LDR process, in accordance with the present invention, where multiple Q-Zip portions containing Q-dots on a single tail are used with microbead capture and a polymerase extension step to distinguish single base differences in nucleic acid molecules.

FIG. 5 is a schematic diagram of an LDR process, in accordance with the present invention, where double Q-Zip portions containing Q-dots on a single tail are used are used with microbead capture and a polymerase extension step to score allele imbalance in a tumor sample compared with a normal sample.

FIG. 6 is a schematic diagram of an LDR process, in accordance with the present invention, where multiple Q-Zip portions containing Q-dots on a single tail are used with microbead capture and a polymerase extension step to score gene copy number in a tumor sample compared with a normal sample.

FIG. 7 is a schematic diagram of an LDR process, in accordance with the present invention, where double Q-Zip portions containing Q-dots on double tails are used with microbead capture and a polymerase extension step to score allele imbalance in a tumor sample compared with a normal sample.

FIG. 8 is a schematic diagram of an LDR process, in accordance with the present invention, where double ligated Q-Zip portions containing Q-dots on double tails are used with microbead capture and a polymerase extension step to score allele imbalance in a tumor sample compared with a normal sample.

FIG. 9 is a schematic diagram of an LDR process, in accordance with the present invention, where double Q-Zip portions containing Q-dots on double tails are used with microbead capture and a polymerase extension step to score gene copy number in a tumor sample compared with a normal sample.

FIG. 10 is a schematic diagram of an LDR process, in accordance with the present invention, where double ligated Q-Zip portions containing Q-dots on double tails are used with microbead capture and a polymerase extension step to score gene copy number in a tumor sample compared with a normal sample.

FIG. 11 is a schematic diagram of an LDR process, in accordance with the present invention, where double Q-Zip portions containing Q-dots on a single tail are used with universal array capture to distinguish single base differences in nucleic acid molecules.

FIG. 12 is a schematic diagram of an LDR process, in accordance with the present invention, where ligated double Q-Zip portions containing Q-dots on a single tail are used with universal array capture to distinguish single base differences in nucleic acid molecules.

FIG. 13 is a schematic diagram of an LDR process, in accordance with the present invention, where double Q-Zip portions containing Q-dots on a single tail are used with universal array capture to score allele imbalance in a tumor sample compared with a normal sample.

FIG. 14 is a schematic diagram of an LDR process, in accordance with the present invention, where ligated double Q-Zip portions containing Q-dots on a single tail are used with universal array capture to score allele imbalance in a tumor sample compared with a normal sample.

FIG. 15 is a schematic diagram of an LDR process, in accordance with the present invention, where double Q-Zip portions containing Q-dots on a single tail are used with universal array capture to score gene copy number in a tumor sample compared with a normal sample.

FIG. 16 is a schematic diagram of an LDR process, in accordance with the present invention, where ligated double Q-Zip portions containing Q-dots on a single tail are used with universal array capture to score gene copy number in a tumor sample compared with a normal sample.

FIG. 17 is a schematic diagram of an LDR process, in accordance with the present invention, where double Q-Zip portions containing Q-dots on double tails are used with microbead capture to distinguish single base differences in nucleic acid molecules.

FIG. 18 is a schematic diagram of an LDR process, in accordance with the present invention, where ligated double Q-Zip portions containing Q-dots on double tails are used with microbead capture to distinguish single base differences in nucleic acid molecules.

FIG. 19 is a schematic diagram of an LDR process, in accordance with the present invention, where double Q-Zip portions containing Q-dots on double tails are used with microbead capture to score allele imbalance in a tumor sample compared with a normal sample.

FIG. 20 is a schematic diagram of an LDR process, in accordance with the present invention, where ligated double Q-Zip portions containing Q-dots on double tails are used with microbead capture to score allele imbalance in a tumor sample compared with a normal sample.

FIG. 21 is a schematic diagram of an LDR process, in accordance with the present invention, where double Q-Zip portions containing Q-dots on double tails are used with microbead capture to score gene copy number in a tumor sample compared with a normal sample.

FIG. 22 is a schematic diagram of an LDR process, in accordance with the present invention, where ligated double Q-Zip portions containing Q-dots on double tails are used with microbead capture to score gene copy number in a tumor sample compared with a normal sample.

FIG. 23 is a schematic diagram of an LDR process, in accordance with the present invention, where double Q-Zip portions containing Q-dots on double tails are used with microbead capture to distinguish single base differences in nucleic acid molecules.

FIG. 24 is a schematic diagram of an LDR process, in accordance with the present invention, where ligated double Q-Zip portions containing Q-dots on double tails are used with microbead capture to distinguish single base differences in nucleic acid molecules.

FIG. 25 is a schematic diagram of an LDR process, in accordance with the present invention, where double Q-Zip portions containing Q-dots on double tails are used with microbead capture to score allele imbalance in a tumor sample compared with a normal sample.

FIG. 26 is a schematic diagram of an LDR process, in accordance with the present invention, where ligated double Q-Zip portions containing Q-dots on double tails are used with microbead capture to score allele imbalance in a tumor sample compared with a normal sample.

FIG. 27 is a schematic diagram of an LDR process, in accordance with the present invention, where double Q-Zip portions containing Q-dots on double tails are used with microbead capture to score gene copy number in a tumor sample compared with a normal sample.

FIG. 28 is a schematic diagram of an LDR process, in accordance with the present invention, where ligated double Q-Zip portions containing Q-dots on double tails are used with microbead capture to score gene copy number in a tumor sample compared with a normal sample.

FIG. 29 is a schematic diagram of an LDR process, in accordance with the present invention, where double Q-Zip portions containing Q-dots on double tails are used with microbead capture to distinguish and quantify splice variants on mRNA.

FIG. 30 is a schematic diagram of an LDR process, in accordance with the present invention, where ligated double Q-Zip portions containing Q-dots on double tails are used with microbead capture to distinguish and quantify splice variants on mRNA.

FIG. 31 is a schematic diagram of an LDR process, in accordance with the present invention, where double Q-Zip portions containing Q-dots on double tails are used with microbead capture to distinguish and quantify splice variants on mRNA in a tumor sample compared with a normal sample.

FIG. 32 is a schematic diagram of an LDR process, in accordance with the present invention, where ligated double Q-Zip portions containing Q-dots on double tails are used with microbead capture to distinguish and quantify splice variants on mRNA in a tumor sample compared with a normal sample.

FIG. 33 is a schematic diagram of an LDR process, in accordance with the present invention, where double Q-Zip portions containing Q-dots on double tails are used with microbead capture to distinguish and quantify splice variants on mRNA.

FIG. 34 is a schematic diagram of an LDR process, in accordance with the present invention, where ligated double Q-Zip portions containing Q-dots on double tails are used with microbead capture to distinguish and quantify splice variants on mRNA.

FIG. 35 is a schematic diagram of an LDR process, in accordance with the present invention, where double Q-Zip portions containing Q-dots on double tails are used with microbead capture to distinguish and quantify splice variants on mRNA in a tumor sample compared with a normal sample.

FIG. 36 is a schematic diagram of an LDR process, in accordance with the present invention, where ligated double Q-Zip portions containing Q-dots on double tails are used with microbead capture to distinguish and quantify splice variants on mRNA in a tumor sample compared with a normal sample.

FIG. 37 is a schematic diagram of an LDR process, in accordance with the present invention, where double Q-Zip portions containing Q-dots on a single tail are used with a universal array to distinguish and quantify splice variants on mRNA.

FIG. 38 is a schematic diagram of an LDR process, in accordance with the present invention, where ligated double Q-Zip portions containing Q-dots on a single tail are used with a universal array to distinguish and quantify splice variants on mRNA.

FIG. 39 is a schematic diagram of an LDR process, in accordance with the present invention, where double Q-Zip portions containing Q-dots on a single tail are used with a universal array to distinguish and quantify splice variants on mRNA in a tumor sample compared with a normal sample.

FIG. 40 is a schematic diagram of an LDR process, in accordance with the present invention, where ligated double Q-Zip portions containing Q-dots on a single tail are used with a universal array to distinguish and quantify splice variants on mRNA in a tumor sample compared with a normal sample.

FIG. 41 is a schematic diagram of an LDR process, in accordance with the present invention, where double Q-Zip portions containing Q-dots on a single tail are used with a universal array to distinguish and quantify splice variants directly on mRNA.

FIG. 42 is a schematic diagram of an LDR process, in accordance with the present invention, where ligated double Q-Zip portions containing Q-dots on a single tail are used with a universal array to distinguish and quantify splice variants directly on mRNA.

FIG. 43 is a schematic diagram of an LDR process, in accordance with the present invention, where double Q-Zip portions containing Q-dots on a single tail are used with a universal array to distinguish and quantify splice variants directly on mRNA in a tumor sample compared with a normal sample.

FIG. 44 is a schematic diagram of an LDR process, in accordance with the present invention, where ligated double Q-Zip portions containing Q-dots on a single tail are used with a universal array to distinguish and quantify splice variants directly on mRNA in a tumor sample compared with a normal sample.

FIG. 45 is a schematic diagram of an LDR process, in accordance with the present invention, where whole genome amplification and double Q-Zip portions containing Q-dots on a single tail are used with universal array capture to distinguish single base differences in nucleic acid molecules.

FIG. 46 is a schematic diagram of an LDR process, in accordance with the present invention, where whole genome amplification and ligated double Q-Zip portions containing Q-dots on a single tail are used with universal array capture to distinguish single base differences in nucleic acid molecules.

FIG. 47 is a schematic diagram of an LDR process, in accordance with the present invention, where whole genome amplification and ligated double Q-Zip portions containing Q-dots on a single tail are used with ligation to a universal array to distinguish single base differences in nucleic acid molecules.

FIG. 48 is a schematic diagram of an LDR process, in accordance with the present invention, where whole genome amplification and double Q-Zip portions containing Q-dots on a single tail are used with universal array capture to score allele imbalance in a tumor sample compared with a normal sample.

FIG. 49 is a schematic diagram of an LDR process, in accordance with the present invention, where whole genome amplification and ligated double Q-Zip portions containing Q-dots on a single tail are used with universal array capture to score allele imbalance in a tumor sample compared with a normal sample.

FIG. 50 is a schematic diagram of an LDR process, in accordance with the present invention, where whole genome amplification and ligated double Q-Zip portions containing Q-dots on a single tail are used with ligation to universal array capture to score allele imbalance in a tumor sample compared with a normal sample.

FIG. 51 is a schematic diagram of an LDR process, in accordance with the present invention, where whole genome amplification and double Q-Zip portions containing Q-dots on a single tail are used with universal array capture to score gene copy number in a tumor sample compared with a normal sample.

FIG. 52 is a schematic diagram of an LDR process, in accordance with the present invention, where whole genome amplification and ligated double Q-Zip portions containing Q-dots on a single tail are used with universal array capture to score gene copy number in a tumor sample compared with a normal sample.

FIG. 53 is a schematic diagram of an LDR process, in accordance with the present invention, where whole genome amplification and ligated double Q-Zip portions containing Q-dots on a single tail are used with ligation to a universal array to score gene copy number in a tumor sample compared with a normal sample.

FIG. 54 is a schematic diagram of an LDR process, in accordance with the present invention, where isothermal amplification and double Q-Zip portions containing Q-dots on a single tail are used with universal array capture to distinguish single base differences in nucleic acid molecules.

FIG. 55 is a schematic diagram of an LDR process, in accordance with the present invention, where isothermal amplification and ligated double Q-Zip portions containing Q-dots on a single tail are used with universal array capture to distinguish single base differences in nucleic acid molecules.

FIG. 56 is a schematic diagram of an LDR process, in accordance with the present invention, where isothermal amplification and ligated double Q-Zip portions containing Q-dots on a single tail are used with ligation to a universal array to distinguish single base differences in nucleic acid molecules.

FIG. 57 is a schematic diagram of an LDR process, in accordance with the present invention, where isothermal amplification and double Q-Zip portions containing Q-dots on a single tail are used with universal array capture to score allele imbalance in a tumor sample compared with a normal sample.

FIG. 58 is a schematic diagram of an LDR process, in accordance with the present invention, where isothermal amplification and ligated double Q-Zip portions containing Q-dots on a single tail are used with universal array capture to score allele imbalance in a tumor sample compared with a normal sample.

FIG. 59 is a schematic diagram of an LDR process, in accordance with the present invention, where isothermal amplification and ligated double Q-Zip portions containing Q-dots on a single tail are used with ligation to a universal array to score allele imbalance in a tumor sample compared with a normal sample.

FIG. 60 is a schematic diagram of an LDR process, in accordance with the present invention, where isothermal amplification and double Q-Zip portions containing Q-dots on a single tail are used with universal array to score gene copy number in a tumor sample compared with a normal sample.

FIG. 61 is a schematic diagram of an LDR process, in accordance with the present invention, where isothermal amplification and ligated double Q-Zip portions containing Q-dots on a single tail are used with universal array capture to score gene copy number in a tumor sample compared with a normal sample.

FIG. 62 is a schematic diagram of an LDR process, in accordance with the present invention, where isothermal amplification and ligated double Q-Zip portions containing Q-dots on a single tail are used with ligation to a universal array to score gene copy number in a tumor sample compared with a normal sample.

FIG. 63 is a schematic diagram of an LDR process, in accordance with the present invention, where isothermal amplification and double Q-Zip portions containing Q-dots on double tails are used with translation oligonucleotide probe (“T-Zip portion”) ligation and universal bead capture with addressable array-specific portions to distinguish single base differences in nucleic acid molecules.

FIG. 64 is a schematic diagram of an LDR process, in accordance with the present invention, where isothermal amplification and ligated double Q-Zip portions containing Q-dots on double tails are used with T-Zip portion ligation and universal bead capture with addressable array-specific portions to distinguish single base differences in nucleic acid molecules.

FIG. 65 is a schematic diagram of an LDR process, in accordance with the present invention, where isothermal amplification and ligated double Q-Zip portions containing Q-dots on double tails are used with T-Zip portion ligation and ligation to universal capture probes with addressable array-specific portions on beads to distinguish single base differences in nucleic acid molecules.

FIG. 66 is a schematic diagram of an LDR process, in accordance with the present invention, where isothermal amplification and double Q-Zip portions containing Q-dots on double tails are used with T-Zip portion ligation and universal bead capture with addressable array-specific portions to score allele imbalance in a tumor sample compared with a normal sample.

FIG. 67 is a schematic diagram of an LDR process, in accordance with the present invention, where isothermal amplification and double Q-Zip portions containing Q-dots on double tails are used with T-Zip portion ligation and universal bead capture with addressable array-specific portions to score allele imbalance in a tumor sample compared with a normal sample.

FIG. 68 is a schematic diagram of an LDR process, in accordance with the present invention, where isothermal amplification and ligated double Q-Zip portions containing Q-dots on double tails are used with T-Zip portion ligation and ligation to universal capture probes with addressable array-specific portions on beads to score allele imbalance in a tumor sample compared with a normal sample.

FIG. 69 is a schematic diagram of an LDR process, in accordance with the present invention, where isothermal amplification and double Q-Zip portions containing Q-dots on double tails are used with T-Zip portion ligation and universal bead capture with addressable array-specific portions to score gene copy number in a tumor sample compared with a normal sample.

FIG. 70 is a schematic diagram of an LDR process, in accordance with the present invention, where isothermal amplification and ligated double Q-Zip portions containing Q-dots on double tails are used with T-Zip portion ligation and universal bead capture with addressable array-specific portions to score gene copy number in a tumor sample compared with a normal sample.

FIG. 71 is a schematic diagram of an LDR process, in accordance with the present invention, where isothermal amplification and ligated double Q-Zip portions containing Q-dots on double tails are used with T-Zip portion ligation and ligation to universal capture probes with addressable array-specific portions on beads to score gene copy number in a tumor sample compared with a normal sample.

FIGS. 72A-C are schematic diagrams showing isothermal representational amplification of genomic DNA. FIG. 72A shows one approach for generating single stranded DNA representations of genomic DNA by using an oligonucleotide containing an infrequent restriction site. FIGS. 72B-C show a second approach for generating single stranded DNA representations of genomic DNA by using an oligonucleotide containing a frequent restriction site.

FIG. 73 is a graph showing the equalization of LDR yields using minicyles, where primers anneal to target 1 (first bar) with 40% efficiency and to target 2 (second bar) with 80% efficiency. For each mini-cycle, the percent yield of a target sequence is calculated from the efficiency of primer ligation multiplied by the amount of free template. For the first mini-cycle, 100% of the template is available. Primers anneal to target 1 with an efficiency of 40% and to target 2 with an efficiency of 80% and ligation occurs. Considering target 1, in the second mini-cycle primers anneal to the remaining 60% of available target with an efficiency of 40%, and ligate. Ligation thus occurs at a further 24% of target 1, leaving 36% of the target available for ligation in the third round, and so on. After 10 mini-cycles, 99% of target 1 and 100% of target 2 have ligation product annealed. The third bar of each mini-cycle shown in FIG. 73 represents the ratio of target 1 to target 2, and indicates that after 10 mini-cycles, both targets are virtually completely annealed to ligated probes.

FIG. 74 is a graph showing the equalization of LDR yields using minicyles, where probes anneal to target 1 (first bar) with 30% efficiency and to target 2 (second bar) with 90% efficiency. For each mini-cycle, the percent yield of a target sequence is calculated from the efficiency of probe ligation multiplied by the amount of free template. For the first mini-cycle, 100% of the template is available. Probes anneal to target 1 with an efficiency of 30% and to target 2 with an efficiency of 90% and ligation occurs. Considering target 1, in the second mini-cycle probes anneal to the remaining 70% of available target 1, with an efficiency of 30%, and ligate. Ligation thus occurs at a further 21% of target 1, leaving 49% of target 1 available for annealing in the third round, and so on. After 10 mini-cycles, 97% of target 1 and 100% of target 2 have ligation product annealed. The third bar of each mini-cycle shown in FIG. 74 represents the ratio of target 1 to target 2, and indicates that after 10 mini-cycles, both targets are virtually completely annealed to ligated probes.

FIG. 75 is a graph showing the equalization of LDR yields using minicyles, where for each target region there is a probe that is specific for the wild type target and one that is specific for the SNP, and where probes anneal to target 1 (first bar) with an overall efficiency of 20% and to target 2 (second bar) with an overall efficiency of 40%. For each mini-cycle, the percent yield of a target sequence is calculated from the efficiency of primer ligation multiplied by the amount of free template. For the first mini-cycle, 100% of the template is available. Primers anneal to target 1 with an efficiency of 40%, to target 2 with an efficiency of 80%, and ligation occurs. However, for each target region there is a primer that is specific for the wild type target and one that is specific for the SNP. Although both primers will compete to anneal to the target region, only one of these probes will ligate successfully. Thus, the overall efficiency of ligation is 20% at target 1 and 40% at target 2. Considering target 1, in the second mini-cycle, probes anneal to the remaining 80% of available target 1 with an efficiency of 20%, and ligate. Ligation thus occurs at a further 16% of target 1, leaving 64% of target 1 available for ligation in the third round, and so on. After 10 mini-cycles, 89% of target 1 and 99% of target 2 have ligation product annealed. The third bar of each mini-cycle shown in FIG. 75 represents the ratio of target 1 to target 2, and indicates that after 10 mini-cycles, comparable proportions of each target are annealed to ligated probes.

FIG. 76 is a graph showing the equalization of LDR yields using minicycles, where for each target region there is a probe that is specific for the wild type target and one that is specific for the SNP, and where probes anneal to target 1 (first bar) with an overall efficiency of 15% and to target 2 (second bar) with an overall efficiency of 45%. For each mini-cycle, the percent yield of a target sequence is calculated from the efficiency of primer ligation multiplied by the amount of free template. For the first mini-cycle, 100% of the template is available. Probes anneal to target 1 with an efficiency of 30%, to target 2 with an efficiency of 90% and ligation occurs. However, for each target region there is a probe that is specific for the wild type target and one that is specific for the SNP. Although both probes will compete to anneal to the target region, only one of these probes will successfully ligate. Thus, the overall efficiency of ligation is 15% at target 1 and 45% at target 2. Considering target 1, in the second mini-cycle, probes anneal to the remaining 85% of available target 1 with an efficiency of 15%, and ligate. Ligation thus occurs at a further 12.75% of target 1, leaving 72.25% of target 1 available for annealing in the third round, and so on. After 10 mini-cycles, 80% of target 1 and 100% of target 2 have ligation product annealed. The third bar of each mini-cycle shown in FIG. 76 represents the ratio of target 1 to target 2, and indicates that after 10 mini-cycles, comparable proportions of each target are annealed to ligated probes.

FIG. 77 is a graph showing the equalization of LDR yields using minicyles, where probes anneal to target 1 (first bar) with 40% efficiency and to target 2 (second bar) with 80% efficiency, and 5% of target molecules dissociate after ligation. For each mini-cycle, the percent yield of a target sequence is calculated from the efficiency of probe ligation multiplied by the amount of free template. For the first mini-cycle, 100% of the template is available. Probes anneal to target 1 with an efficiency of 40%, to target 2 with an efficiency of 80%, and ligation occurs. However, following ligation, 5% of product molecules dissociate. Considering target 1, 62% is available for further annealing in the second mini-cycle, and this proceeds again with an efficiency of 40%. Following ligation, 5% of ligation product dissociates, leaving 37% of target 1 available for further annealing in the third round, and so on. Because ligation product molecules dissociate from the target after each mini-cycle, the total yield can exceed the amount represented by 100% of the target region. After 10 mini-cycles, the amount of ligation product from target 1 corresponds to 128% of the total region, and the amount from target 2 corresponds to 142% of the total region. The third bar of each mini-cycle shown in FIG. 77 represents the ratio of target 1 to target 2, and indicates that after 10 mini-cycles, comparable amounts of product have been produced from each target.

FIG. 78 is a graph showing the equalization of LDR yields using minicyles, where probes anneal to target 1 (first bar) with 30% efficiency and to target 2 (second bar) with 90% efficiency, and 5% of product molecules dissociate after ligation. For each mini-cycle, the percent yield of a target sequence is calculated from the efficiency of probe ligation multiplied by the amount of free template. For the first mini-cycle, 100% of the template is available. Probes anneal to target 1 with an efficiency of 30%, to target 2 with an efficiency of 90%, and ligation occurs. However, following ligation, 5% of product molecules dissociate. Considering target 1, 71.5% is available for further annealing in the second mini-cycle, and this proceeds again with an efficiency of 40%. Following ligation, 5% of ligation product dissociates, leaving 49% of target 1 available for further annealing in the third round, and so on. Because ligation product molecules dissociates from the target after each mini-cycle, the total yield can exceed the amount represented by 100% of the target region. After 10 mini-cycles, the amount of ligation product from target 1 corresponds to 120% of the total region, and the amount from target 2 corresponds to 144% of the total region. The third bar of each mini-cycle shown in FIG. 78 represents the ratio of target 1 to target 2, and indicates that after 10 mini-cycles, comparable amounts of product have been produced from each target.

FIG. 79 is a graph showing the equalization of LDR yields using minicyles, where for each target region there is a probe that is specific for the wild type target and one that is specific for the SNP, and where probes anneal to target 1 (first bar) with an overall efficiency of 20% and to target 2 (second bar) with an overall efficiency of 40%, and 5% of product molecules dissociate after ligation. For each mini-cycle, the percent yield of each target sequence is calculated from the efficiency of probe ligation multiplied by the amount of free template. For the first mini-cycle, 100% of the template is available. Probes anneal to target 1 with an efficiency of 40%, to target 2 with an efficiency of 80% and ligation occurs. However, for each target region there is a probe that is specific for the wild type target and one that is specific for the SNP. Although both probes will compete to anneal to the target region, only one of these probes will successfully ligate. Thus, the overall efficiency of ligation is 20% at target 1 and 40% at target 2. Once ligation has occurred, 5% of product molecules dissociate. Considering target 1, in the second mini-cycle, probes anneal to the remaining 81% of available target 1, again with an efficiency of 20%, and ligate. Following ligation, 5% of ligation product dissociates, leaving 66% of target 1 available for further annealing in the third round, and so on. Because ligation product dissociates from the target after each mini-cycle, the total yield can exceed the amount represented by 100% of the target region. After 10 mini-cycles, the amount of ligation product from target 1 corresponds to 103% of the total region, and the amount from target 2 corresponds to 128% of the total region. The third bar of each mini-cycle shown in FIG. 79 represents the ratio of target 1 to target 2, and indicates that after 10 mini-cycles, comparable amounts of product have been produced from each target.

FIG. 80 is a graph showing the equalization of LDR yields using minicyles, where for each target region there is a probe that is specific for the wild type target and one that is specific for the SNP, and where probes anneal to target 1 (first bar) with an overall efficiency of 15% and to target 2 (second bar) with an overall efficiency of 45%, and 5% of product molecules dissociate after ligation. For each mini-cycle, the percent yield of each target sequence is calculated from the efficiency of probe ligation multiplied by the amount of free template. For the first mini-cycle, 100% of the template is available. Probes anneal to target 1 with an efficiency of 30% and to target 2 with an efficiency of 90%. However, for each target region there is a probe that is specific for the wild type target and one that is specific for the SNP. Although both probes will compete to anneal to the target region, only one of these probes will successfully ligate. Thus, the overall efficiency of ligation is 15% at target 1 and 45% at target 2. Once ligation has occurred, 5% of product molecules dissociate. Considering target 1, in the second mini-cycle, probes anneal to the remaining 86% of available target 1, again with an efficiency of 15%, and ligate. Following ligation, 5% of ligation product dissociates, leaving 72% of target 1 available for further annealing in the third round, and so on. Because ligation product dissociates from the target after each mini-cycle, the total yield can exceed the amount represented by 100% of the target region. After 10 mini-cycles, the amount of ligation product from target 1 corresponds to 90% of the total region, and the amount from target 2 corresponds to 131% of the total region. The third bar of each mini-cycle shown in FIG. 80 represents the ratio of target 1 to target 2, and indicates that after 10 mini-cycles, comparable amounts of product have been produced from both targets.

FIG. 81 is a graph showing the Tm values of Q-Zip portions containing Q-dots that are 16mers, where the Tm values are from 63° C. to 69.5° C.

FIG. 82 is a graph showing the Tm values of T-Zip portions that are 16mers, where the Tm values are from 66.2° C. to 67.4° C.

FIG. 83 is an agarose gel photograph showing product yields for whole genome amplification of 100 ng of genomic human DNA. The colors have been inverted for clarity. Probe mixes 1, labeled AA probe mix, and 2, labeled CC probe mix, both contain semi-random hexamers, but represent different approaches to the probe design. No template DNA was added to the amplification mixture displayed in the left hand two lanes to confirm that, under the reaction conditions used, no detectable primer dimerism was occurring. The center two lanes show the product obtained from whole genome amplification using the polymerase buffer supplied by the manufacturer (New England Biolabs), and the right hand two lanes contain products from the amplification using optimized buffer conditions. The gel is 1.5% agarose, run for 40 minutes at 6V/cm in 1×TBE buffer. Due to the hyperbranched nature of the product molecules, the DNA does not migrate as far as would be anticipated for linear DNA of the same length.

FIG. 84 is a polyacrylamide gel showing a test of synthetic oligonucleotides with different blocking groups for resistance to Lambda Exonuclease or Exonuclease I. 3.5 ng of substrate was incubated at 37° C. for 1 h in a 30-μl reaction volume in the presence or absence of increasing amounts (units) of Exonuclease (5′→3′ Lambda Exonuclease and/or 3′→5′ Exonuclease I). Prior to the digestion reaction, the 5′ end of substrate XS 3′ F1 (μg) was phosphorylated by 10 units of T4 polynucleotide kinase in the presence of 10 mM ATP at 37° C. for 1 h in a 25 μl reaction volume. Aliquots of the digestion reactions were electrophoresed in a 10% denaturing polyacrylamide gel, and detected using filter C setting on an ABI 377 DNA sequencer (blue signal). 3.5 ng oligonucleotide diluted in water was used as a control (lane “-”).

FIG. 85 is a polyacrylamide gel showing a test of synthetic oligonucleotides with different blocking groups for resistance to Exonuclease I. 15 ng of substrate was incubated at 37° C. for 1 h in a 15 μl reaction volume in the presence or absence of 3′→5′ Exonuclease I. Aliquots of the digestion reactions were electrophoresed in a 10% denaturing polyacrylamide gel, and detected using filter C setting on an ABI 377 DNA sequencer (blue signal). 15 ng oligonucleotide diluted in water was used as a control (lane “-”).

FIG. 86 is a polyacrylamide gel showing a test of synthetic oligonucleotides with different blocking groups for resistance to Exonuclease III. 15 ng of substrate was incubated at 37° C. for 1 h in a 15 μl reaction volume in the presence or absence of 3′→5′ Exonuclease III. Aliquots of the digestion reactions were electrophoresed in a 10% denaturing polyacrylamide gel, and detected using filter C setting on an ABI 377 DNA sequencer (blue signal). 15 ng oligonucleotide diluted in water was used as a control (lane “-”).

FIG. 87 is a polyacrylamide gel showing a test of synthetic oligonucleotides with different blocking groups for resistance to Lambda Exonuclease. 3.5 ng of substrate was incubated at 37° C. for 1 h in a 30 μl reaction volume in the presence or absence of 5′ 3′ Lambda Exonuclease. Prior to the digestion reaction, the 5′end of substrate XS 3′F1 (1 μg) was phosphorylated by 10 units of T4 polynucleotide kinase in the presence of 10 mM ATP at 37° C. for 1 h in a 25 μl reaction volume. Aliquots of the digestion reactions were electrophoresed in a 10% denaturing polyacrylamide gel, and detected using filter C setting on an ABI 377 DNA sequencer (blue signal). 3.5 ng oligonucleotide diluted in water was used as a control (lane “-”).

FIG. 88 is a polyacrylamide gel showing a test of synthetic oligonucleotides with different blocking groups for resistance to Exonuclease VII. 15 ng of substrate was incubated at 37° C. for 1 h in a 15 μl reaction volume in the presence or absence of 5′→3′ and 3→5′ Exonuclease VII. Aliquots of the digestion reactions were electrophoresed in a 10% denaturing polyacrylamide gel, and detected using filter C setting on an ABI 377 DNA sequencer (blue signal). 15 ng oligonucleotide diluted in water was used as a control (lane “-”).

FIG. 89A-B illustrate a procedure for preparing Q-dots with monovalent tails.

FIGS. 89A-B are flow diagrams showing the preparation of Q-dots with monovalent tails. Two exemplary methods for preparing Q-dots with monovalent tails are shown. In either case, the first step is preparation of magnetic beads bearing a single complementary Q-Zip-oligonucleotide, as shown in FIGS. 89A and B. Magnetic beads containing COOH groups are activated with 1-[3-(Dimethylamino)propyl]-3-ethylcarbodiimide (EDC) in imidazole buffer and 5′-amino complementary Q-Zips are linked covalently to the magnetic beads via formation of an amide bond. (For attaching Q-Zips on the 3′ end, 3′ amino complementary Q-Zips are used.) The magnetic beads are captured and washed extensively to remove free unreacted oligonucleotides. The degree of functionalization of the magnetic beads can be tuned by using another amine such as ethanolamine or 1-aminohexanol along with the oligonucleotide.

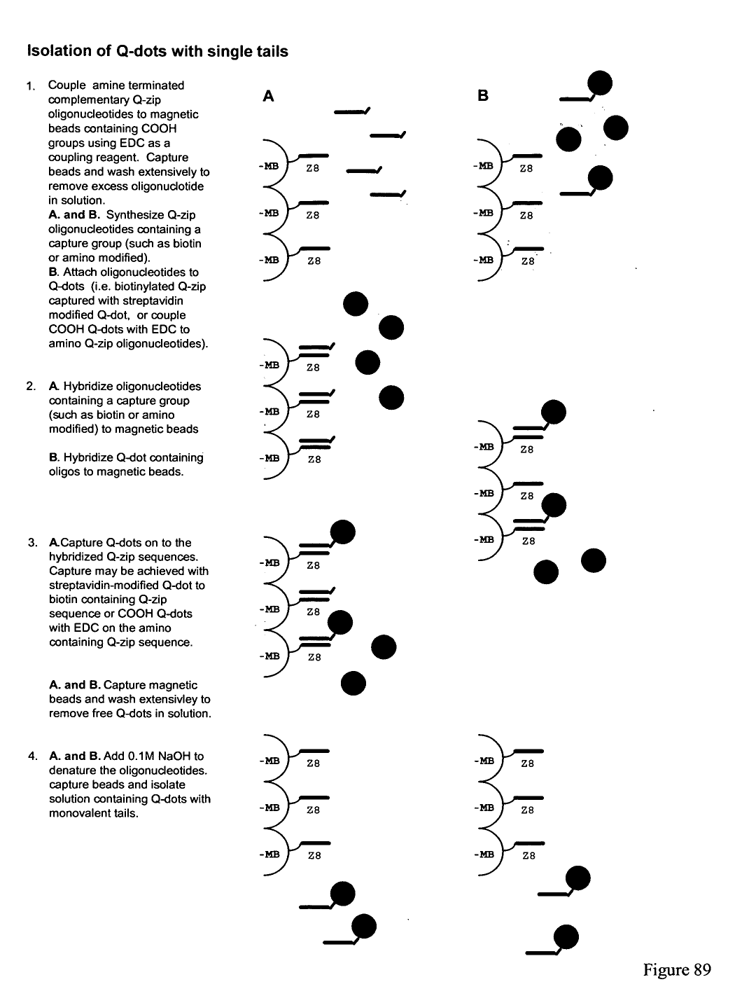

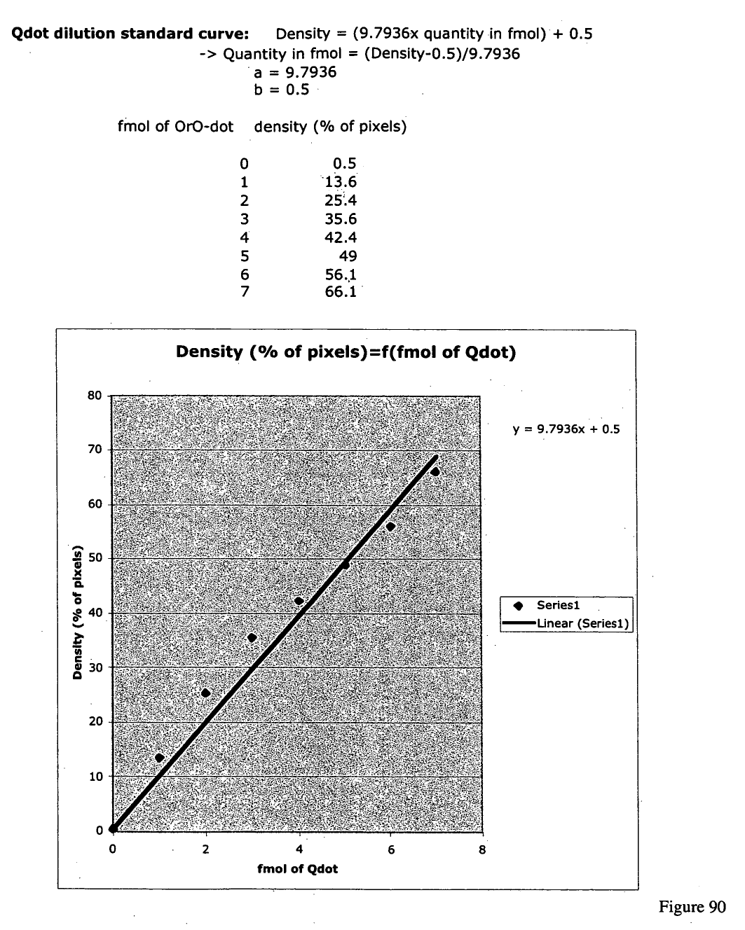

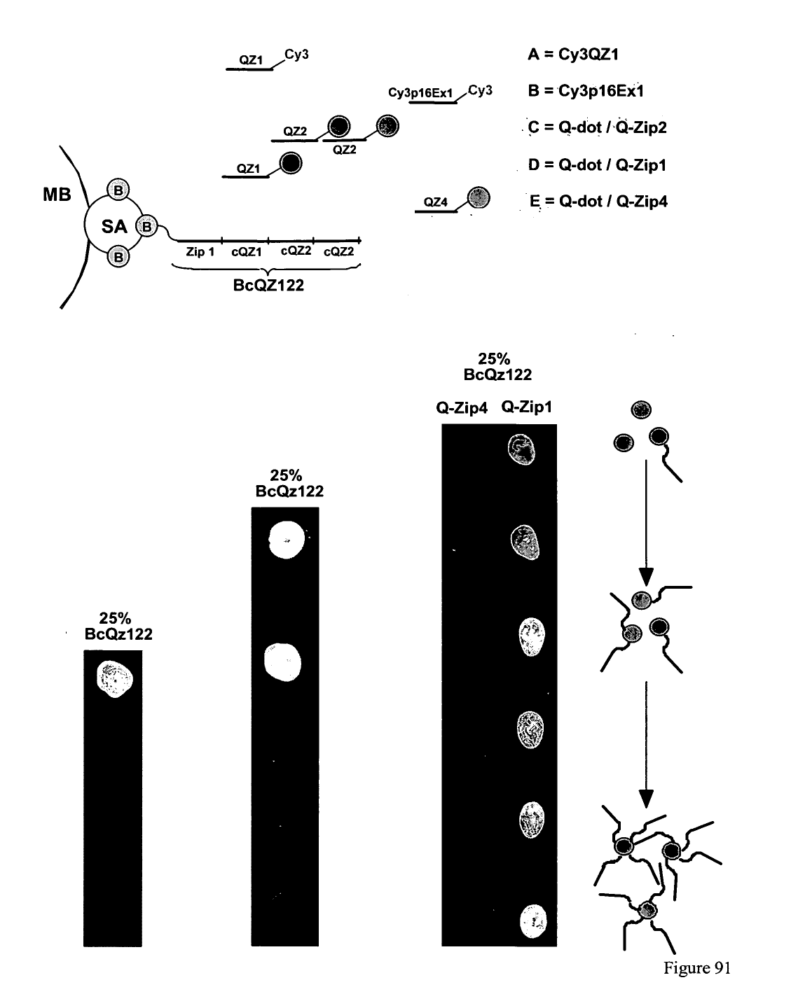

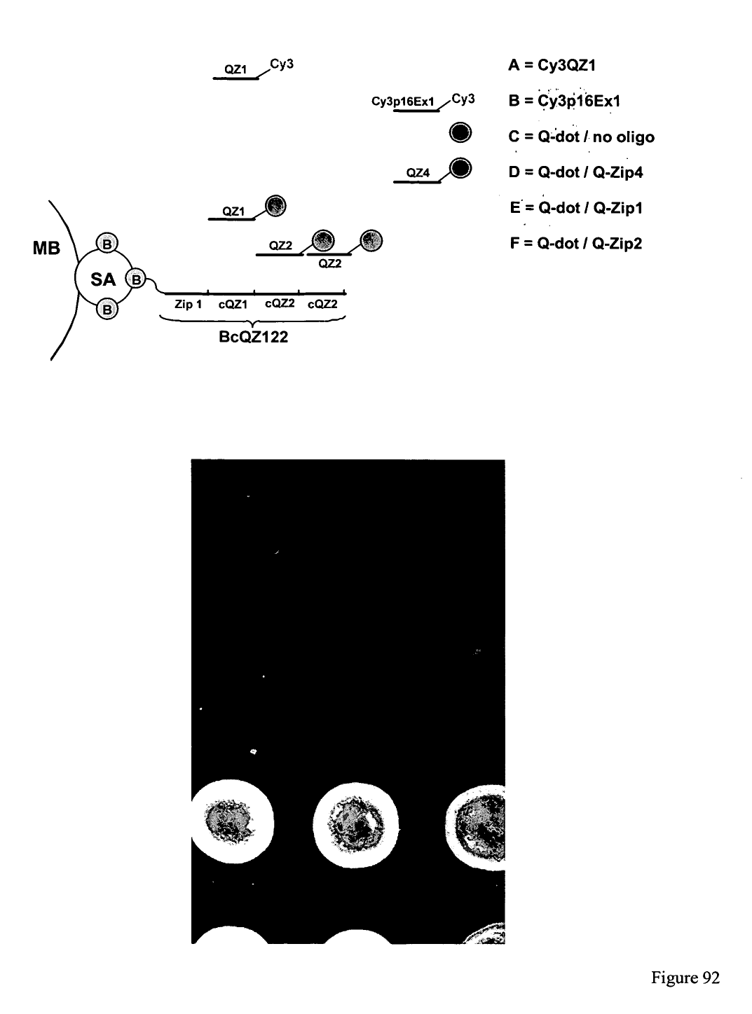

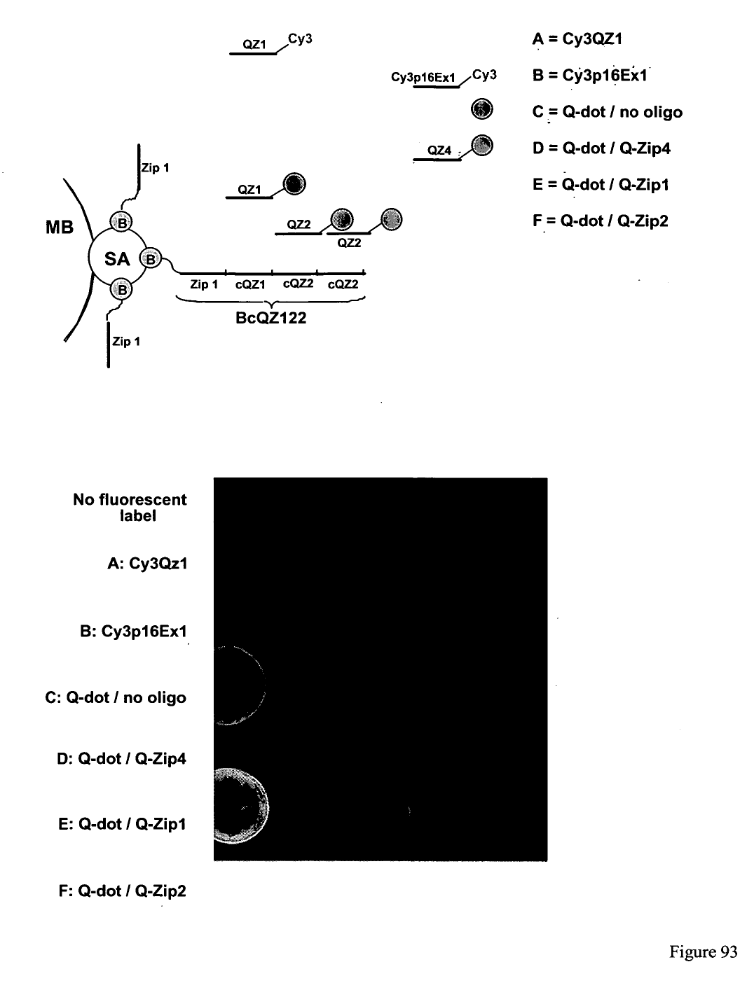

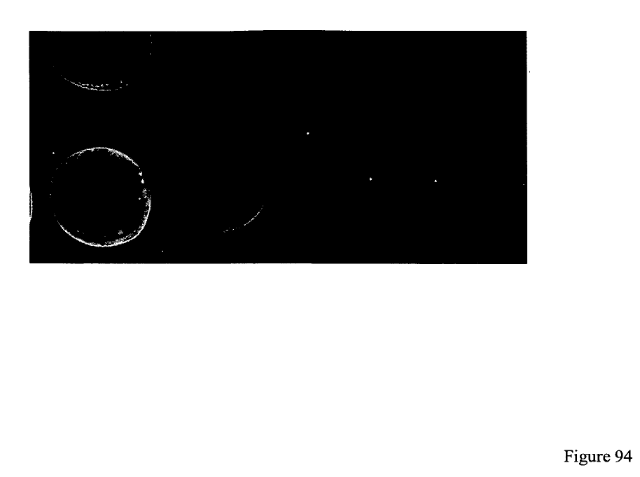

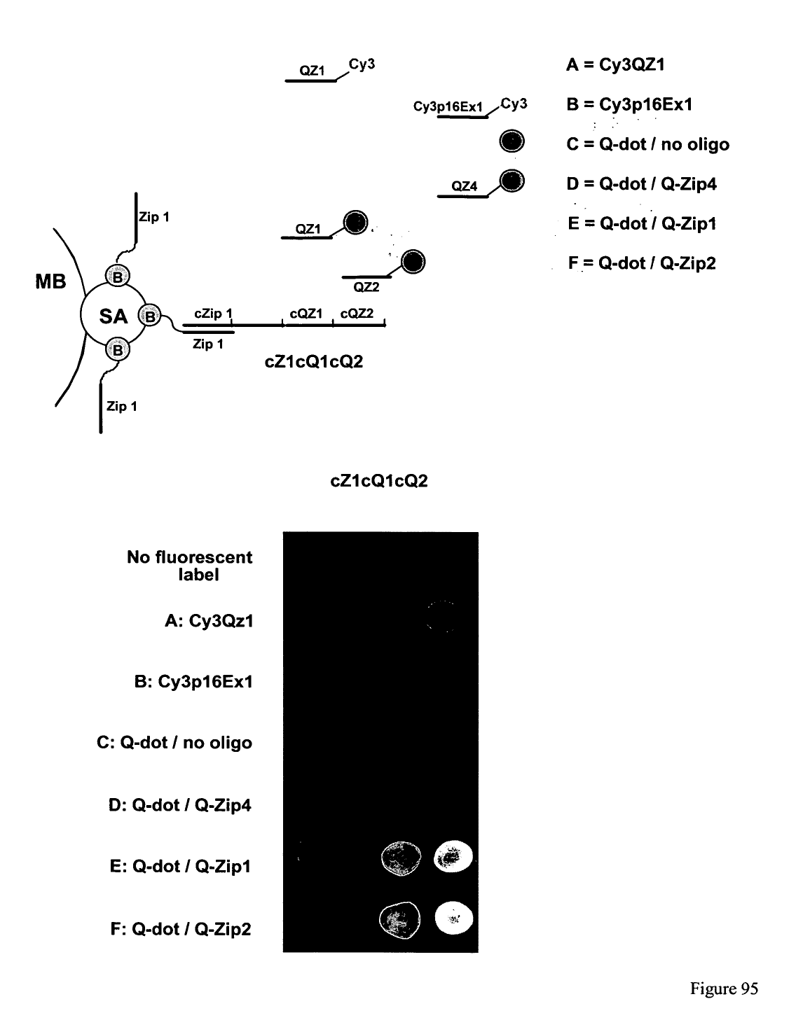

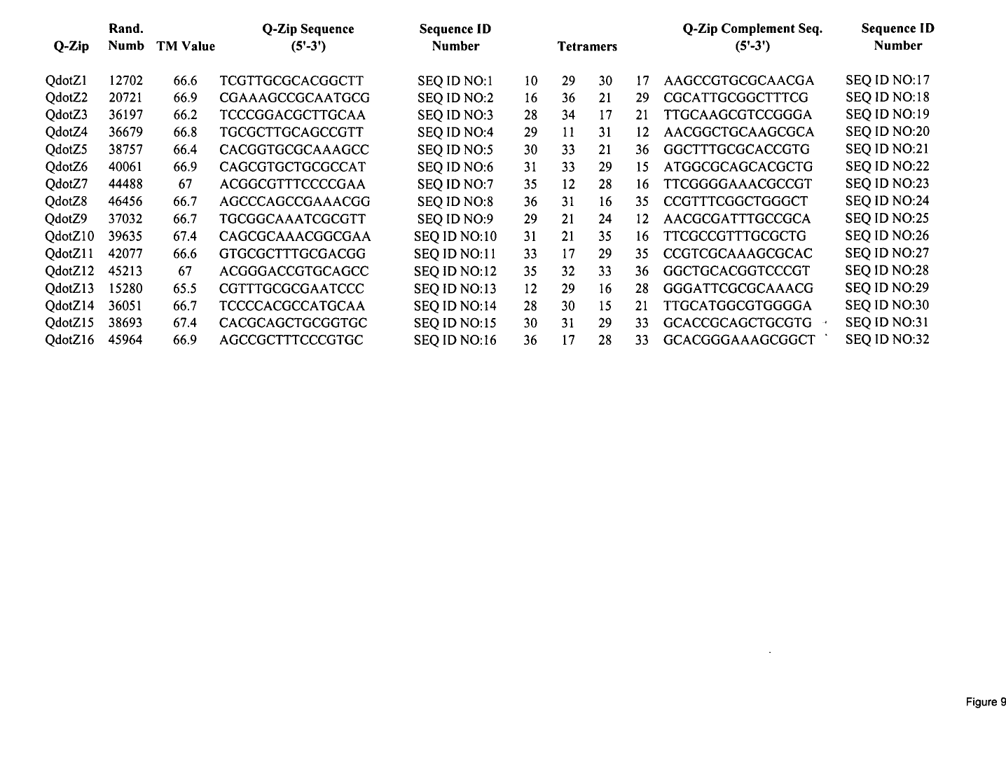

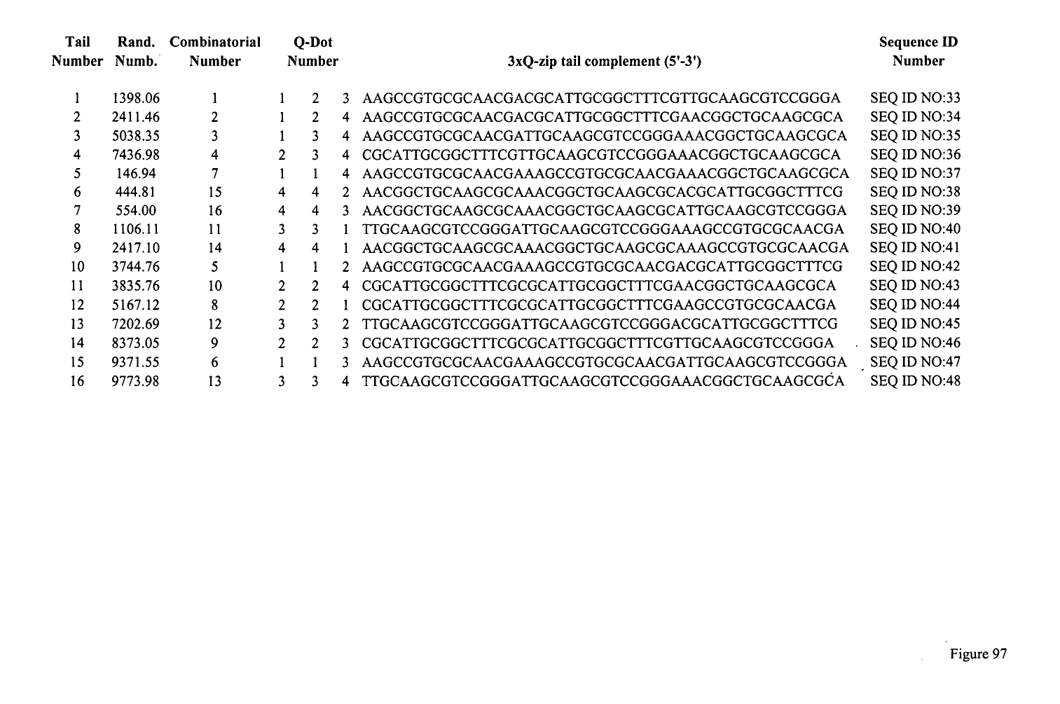

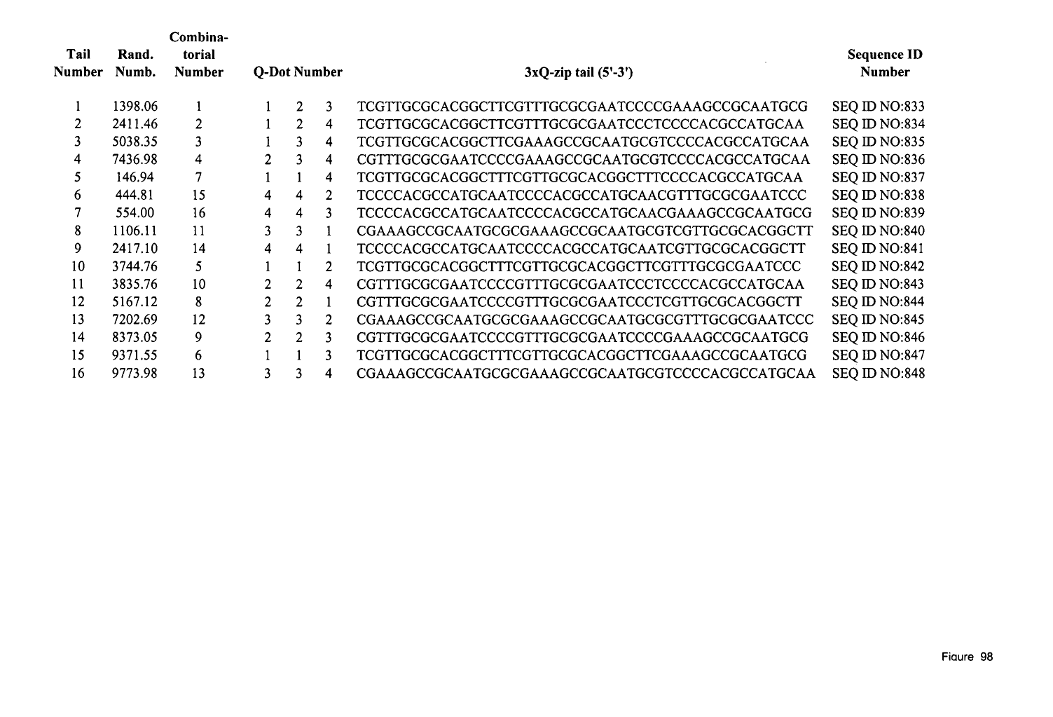

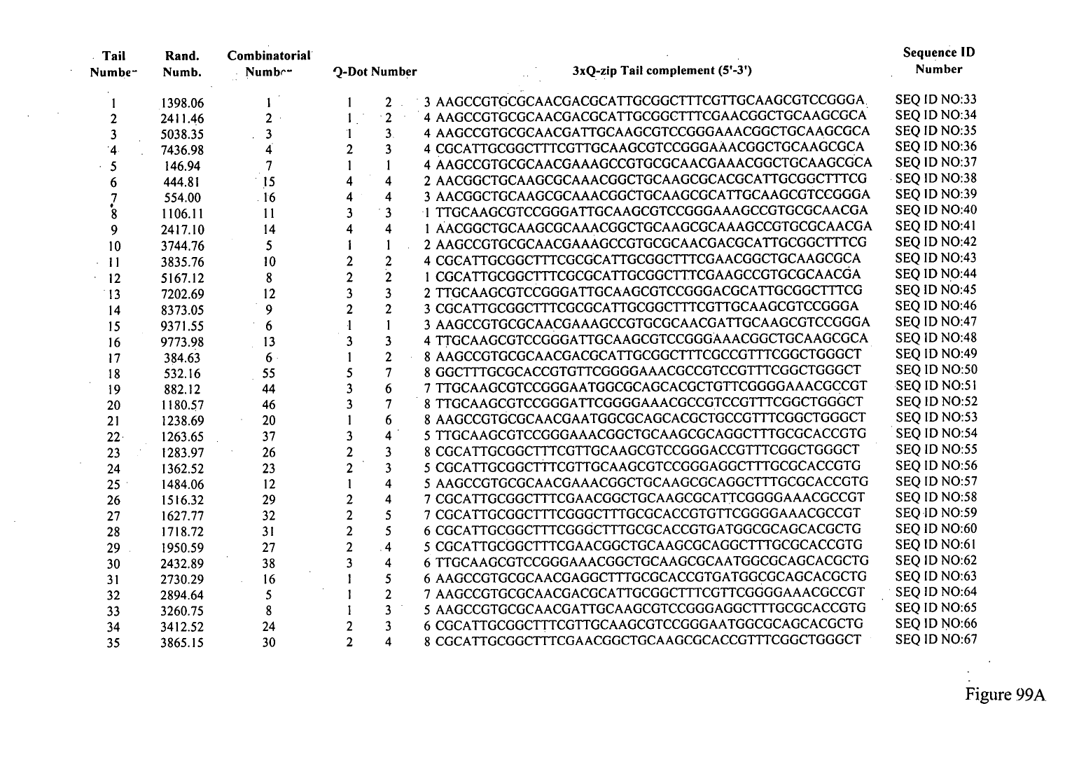

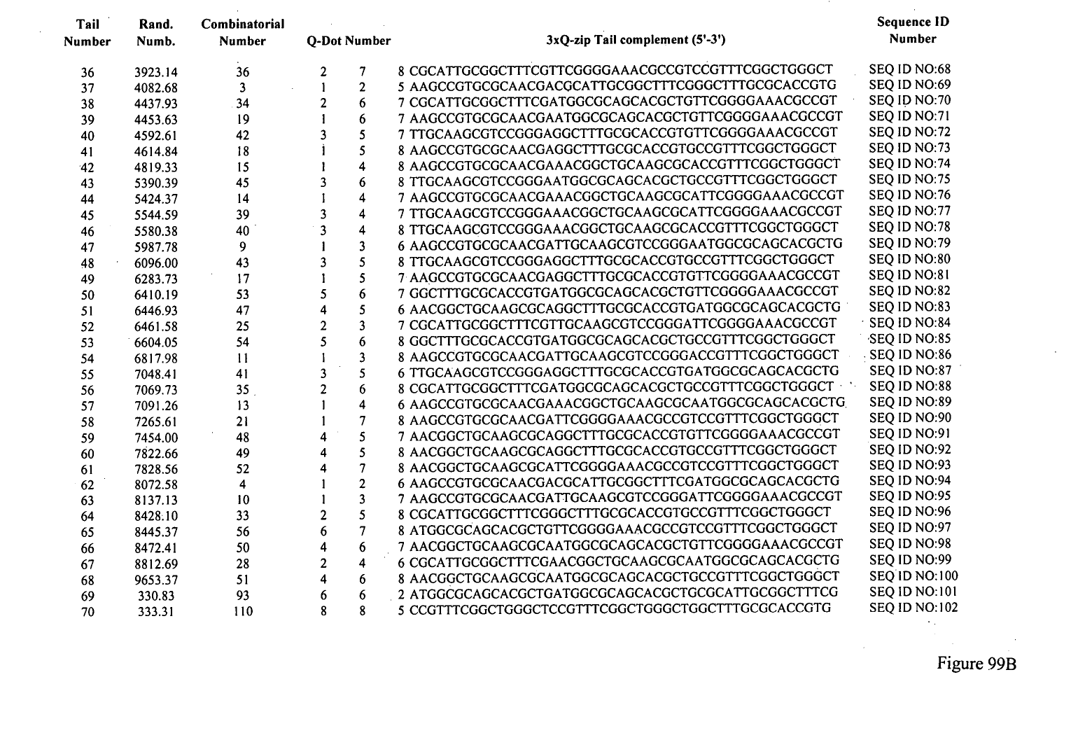

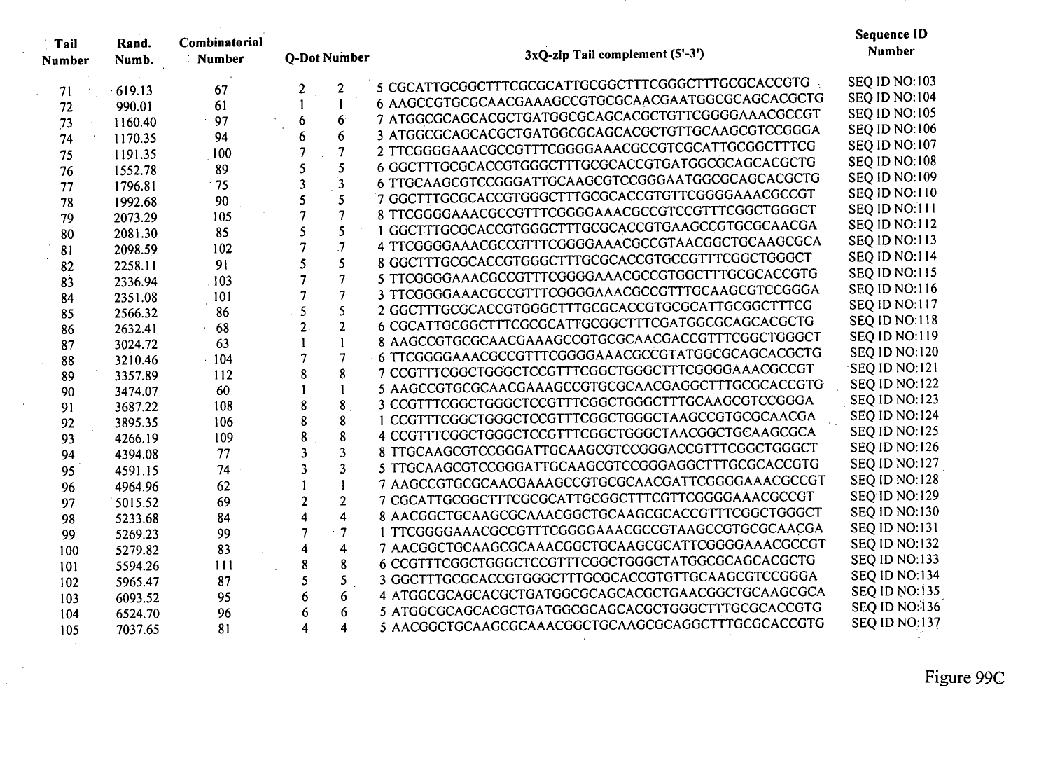

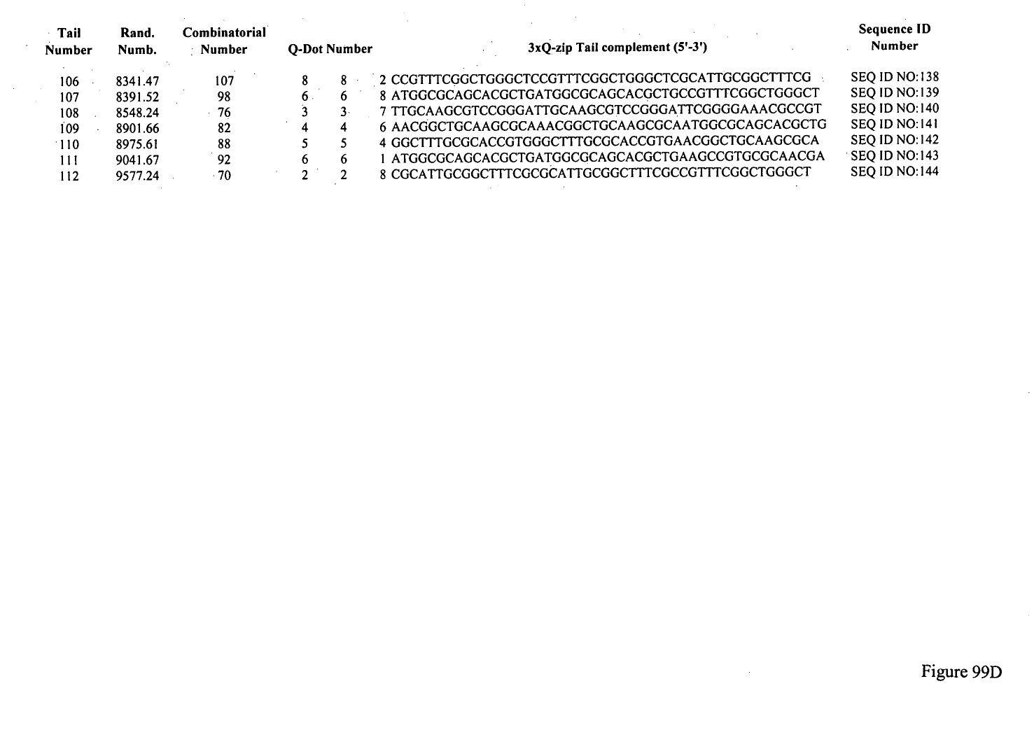

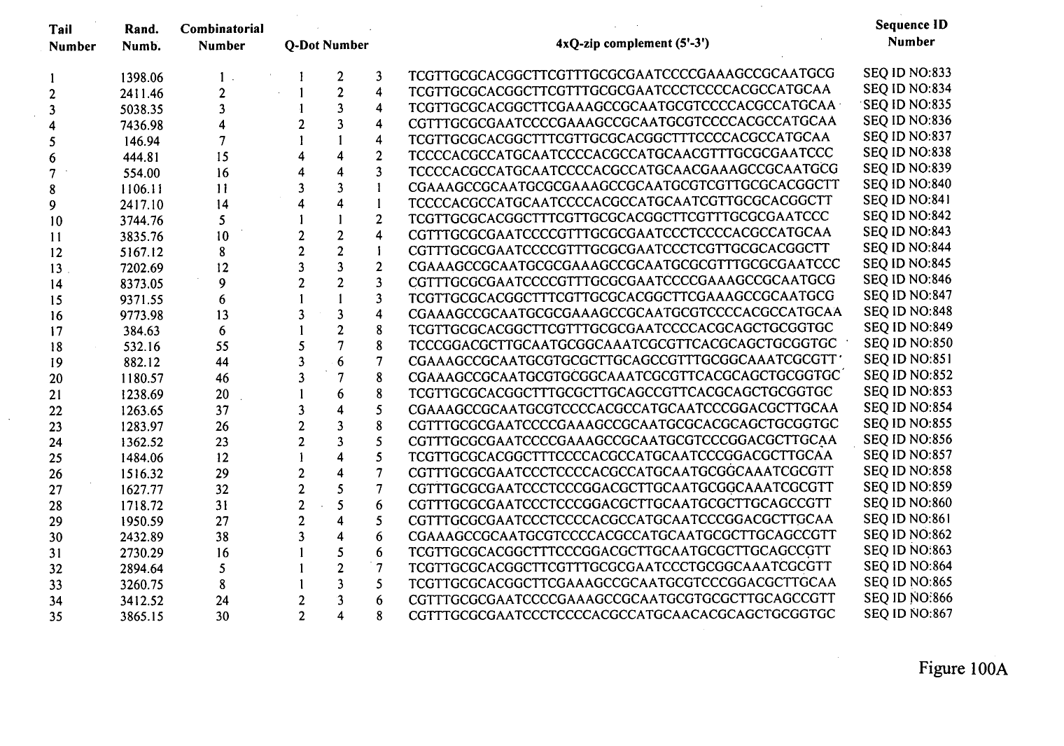

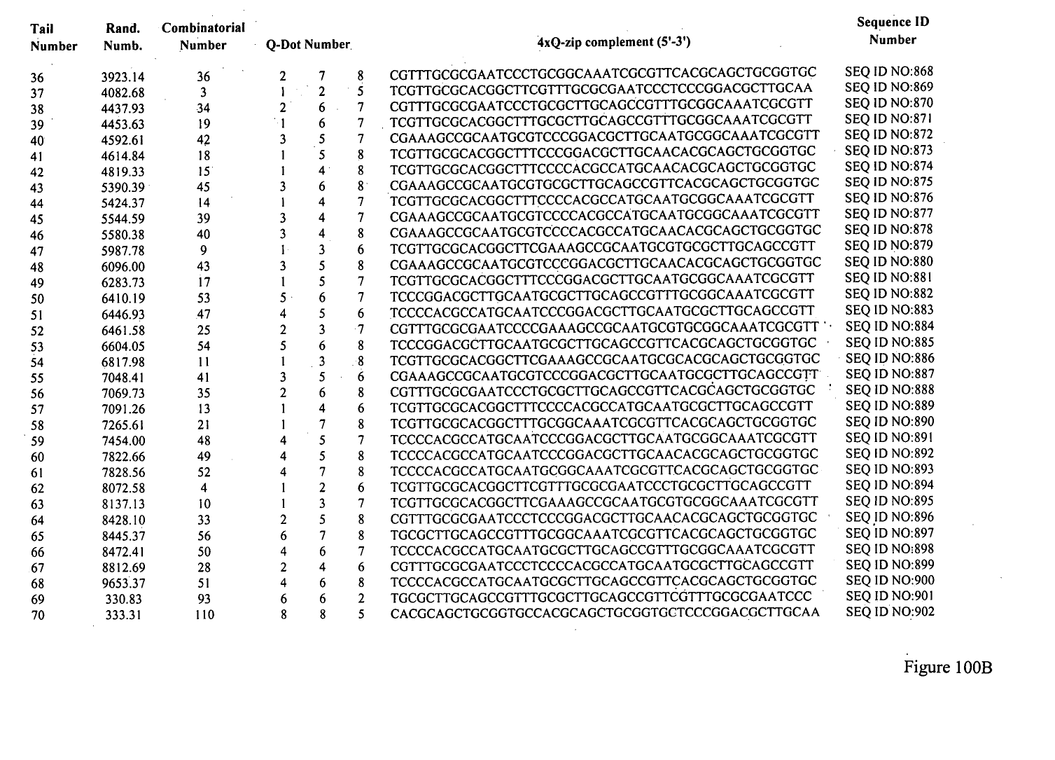

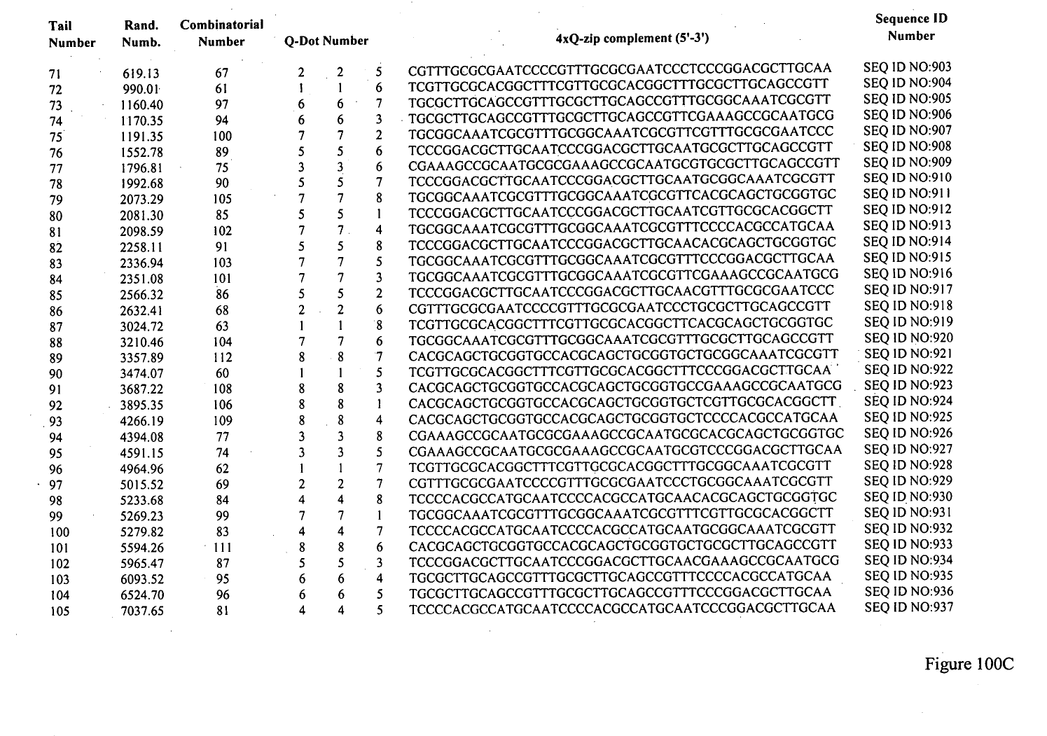

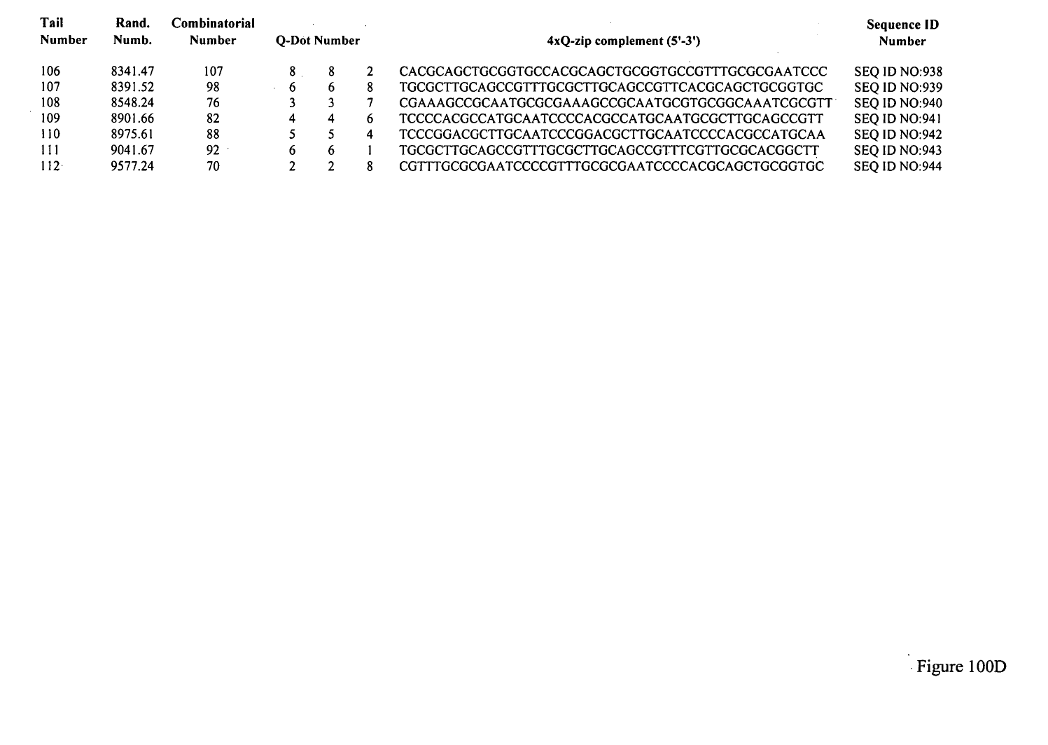

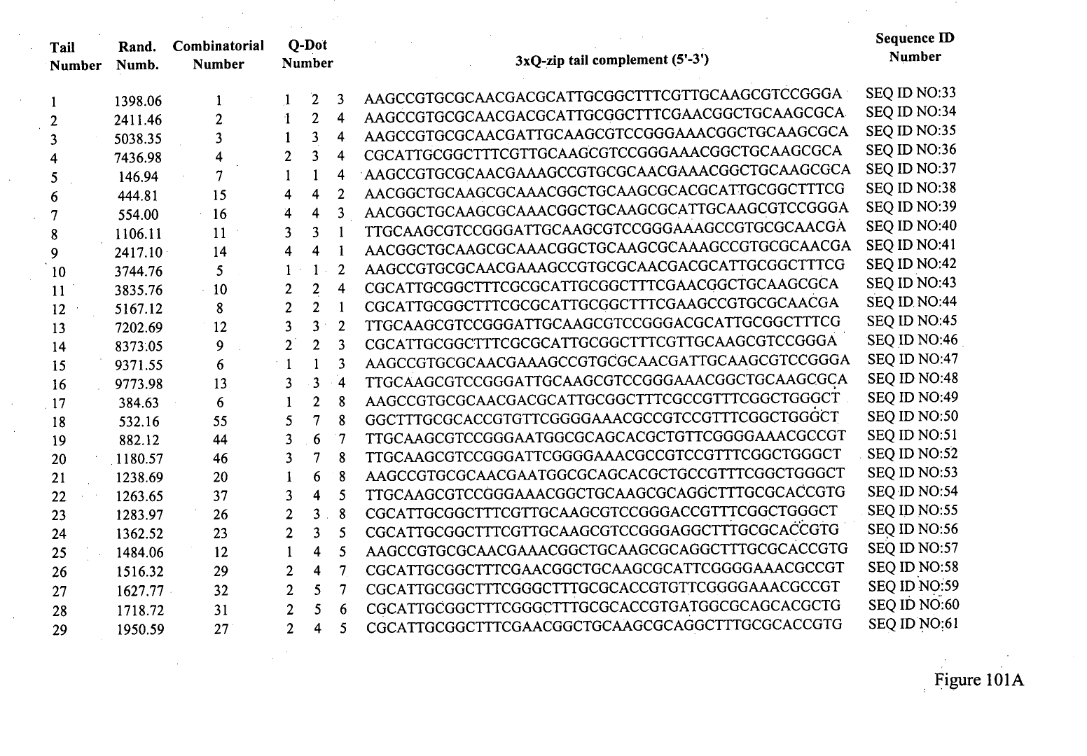

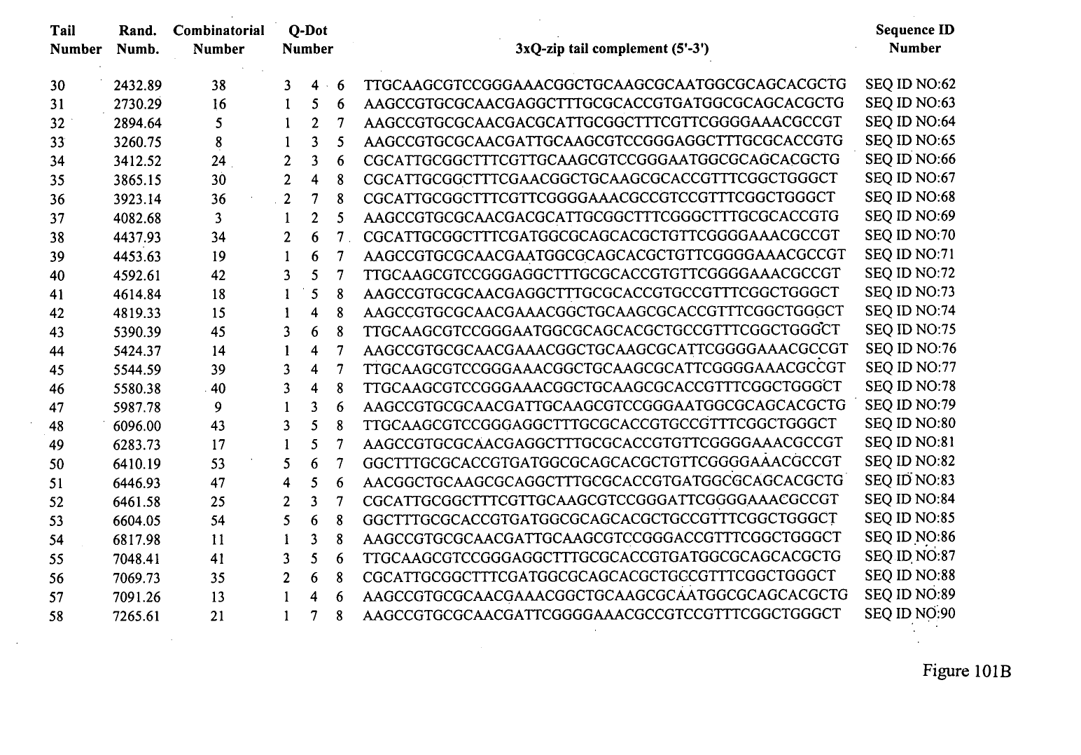

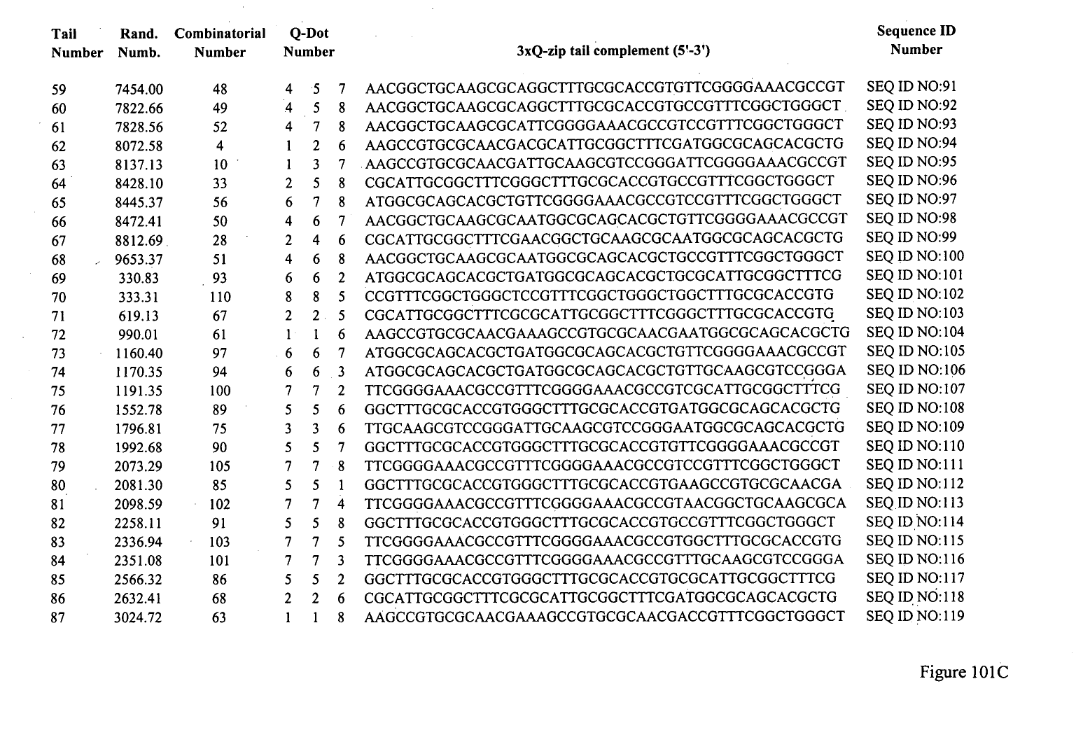

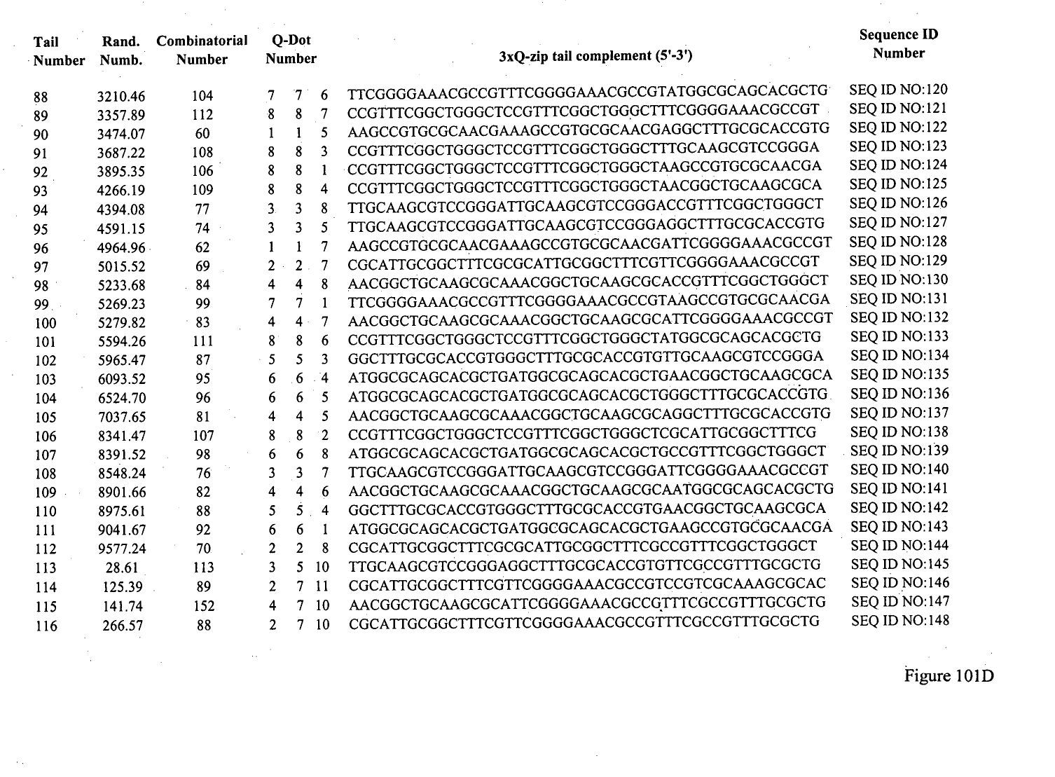

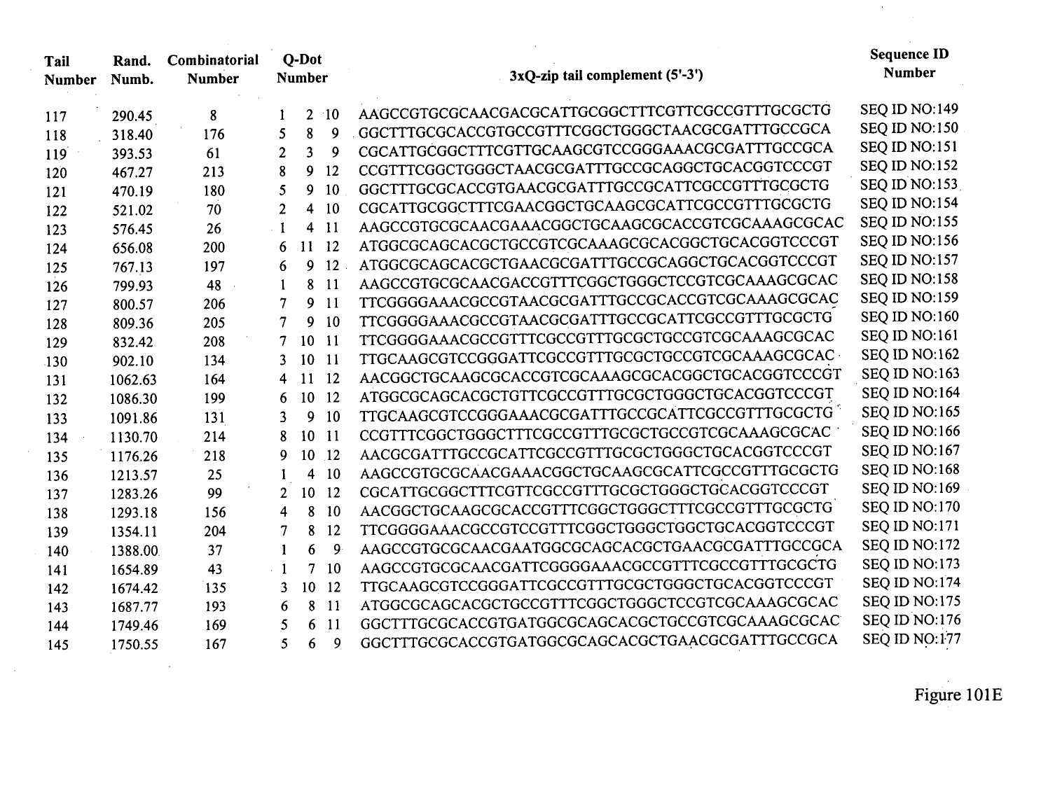

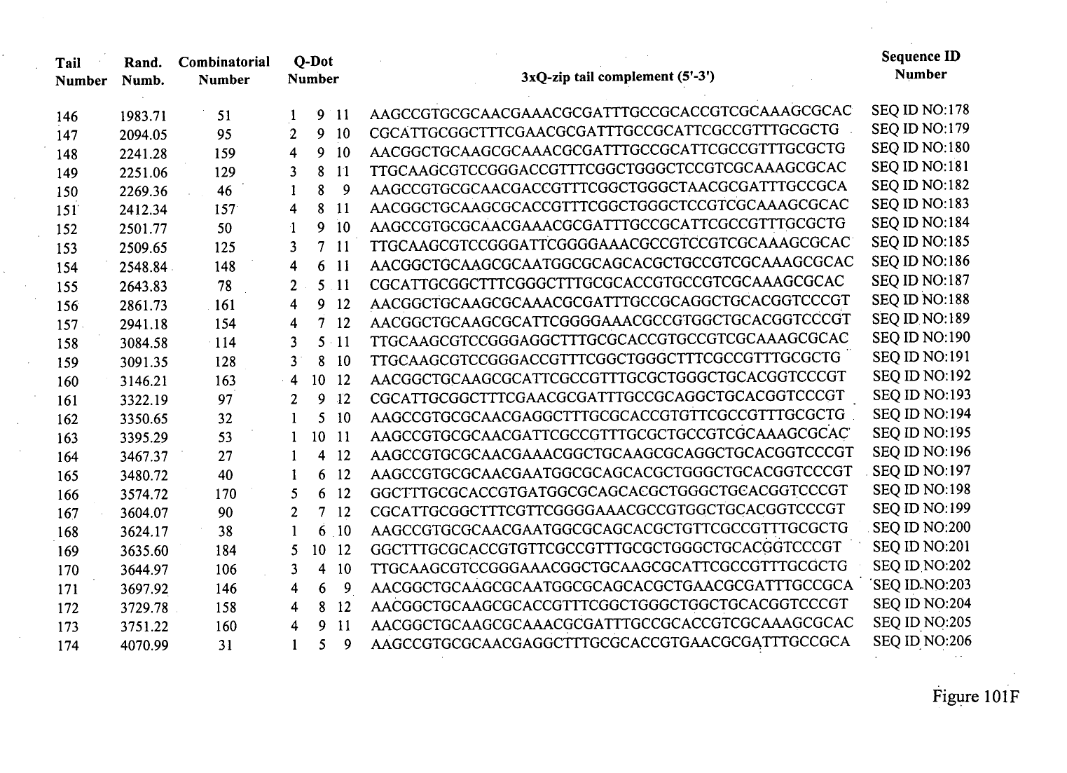

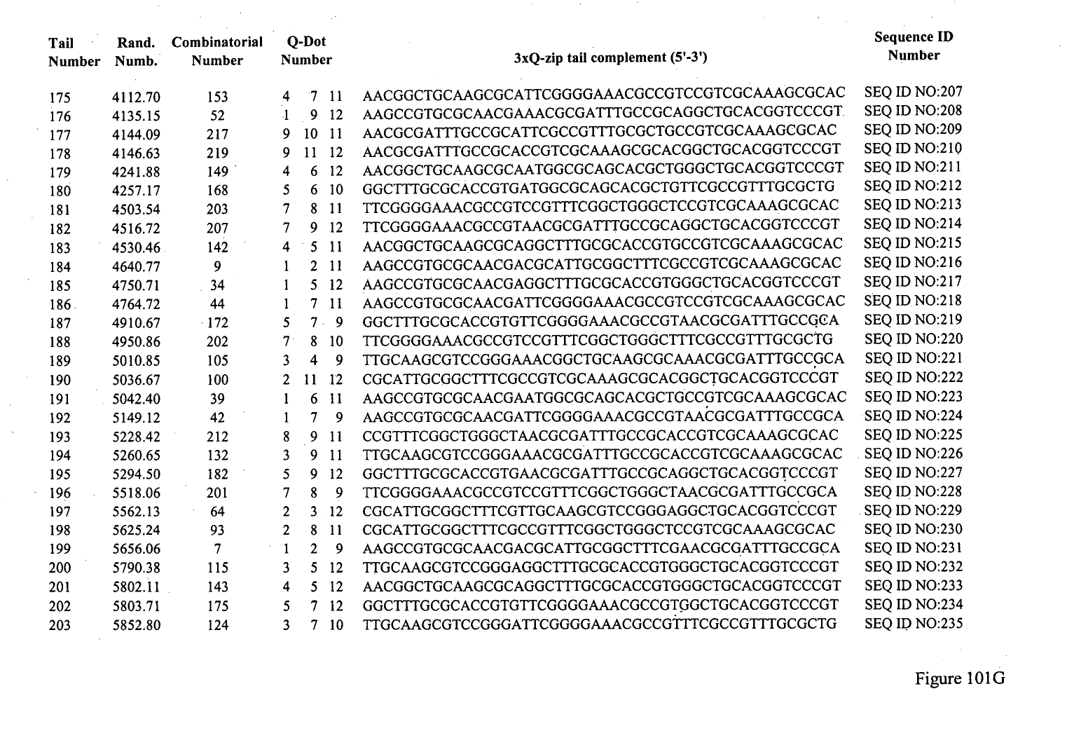

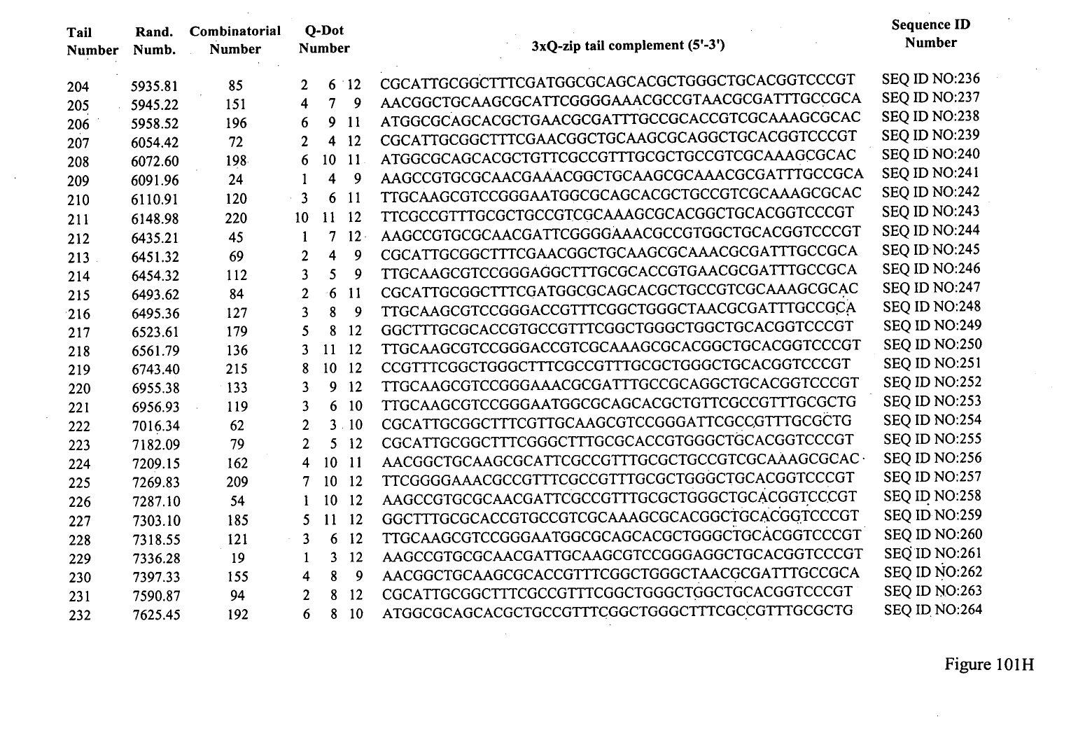

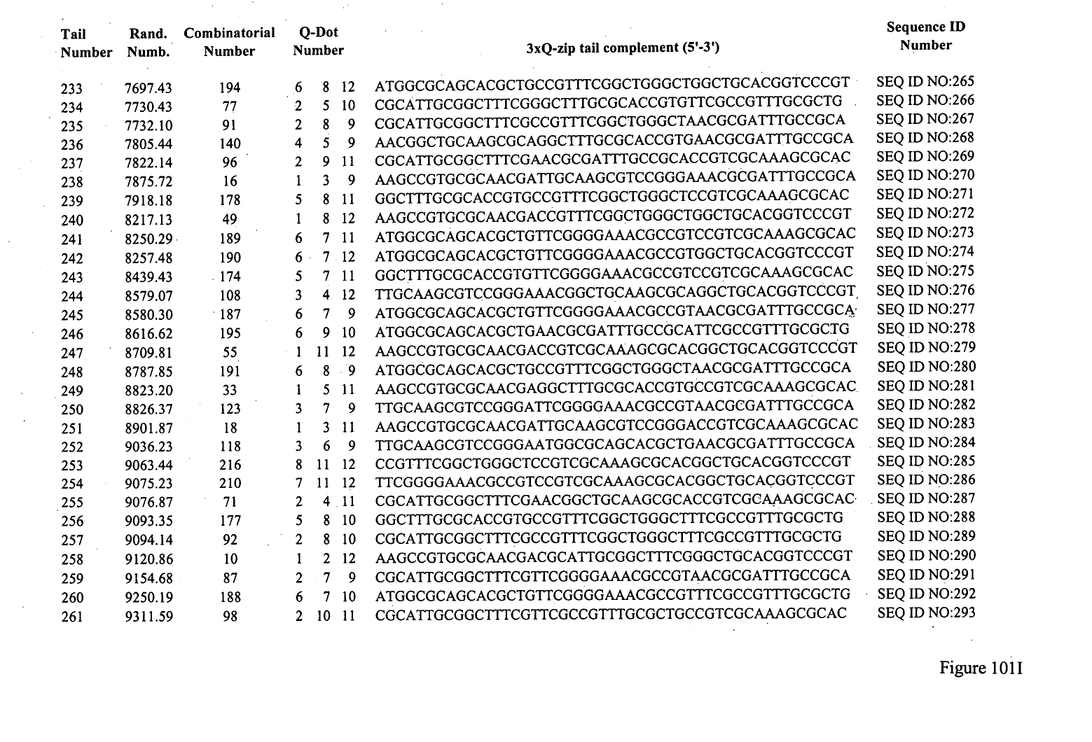

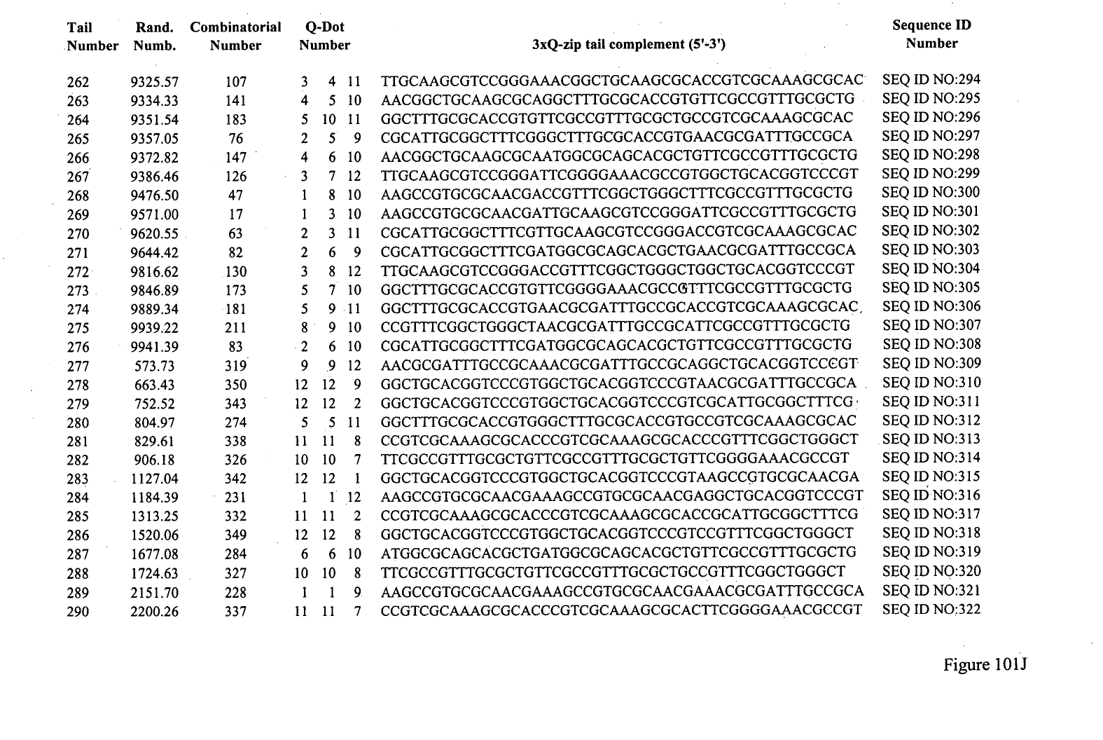

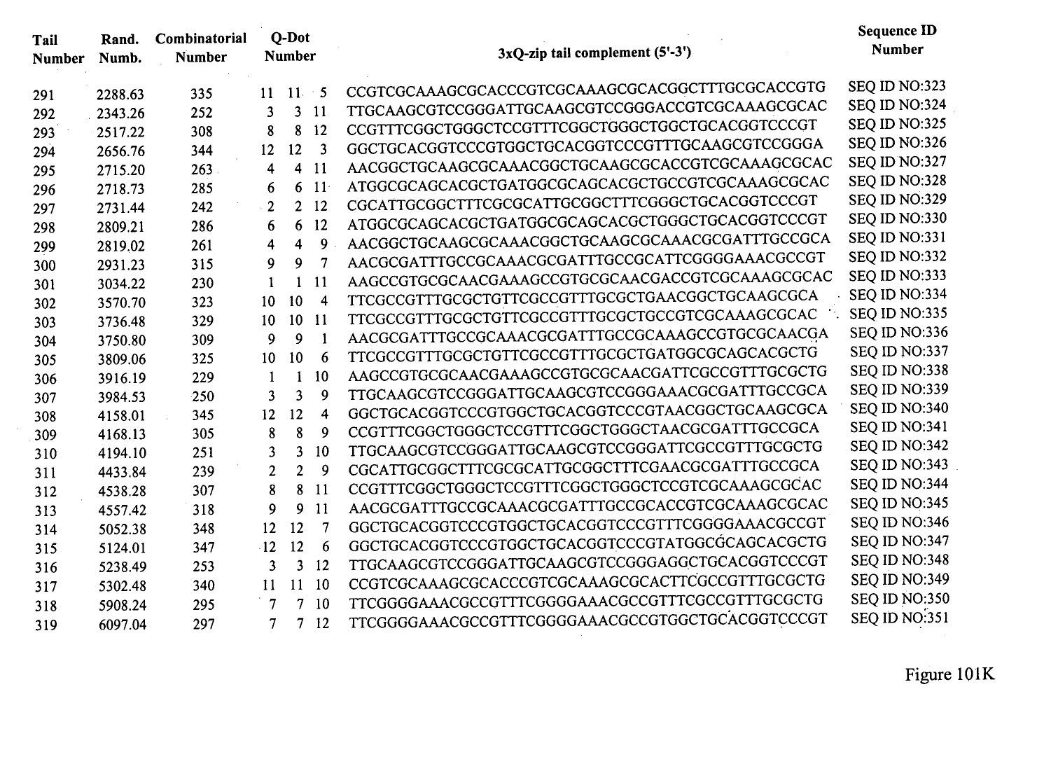

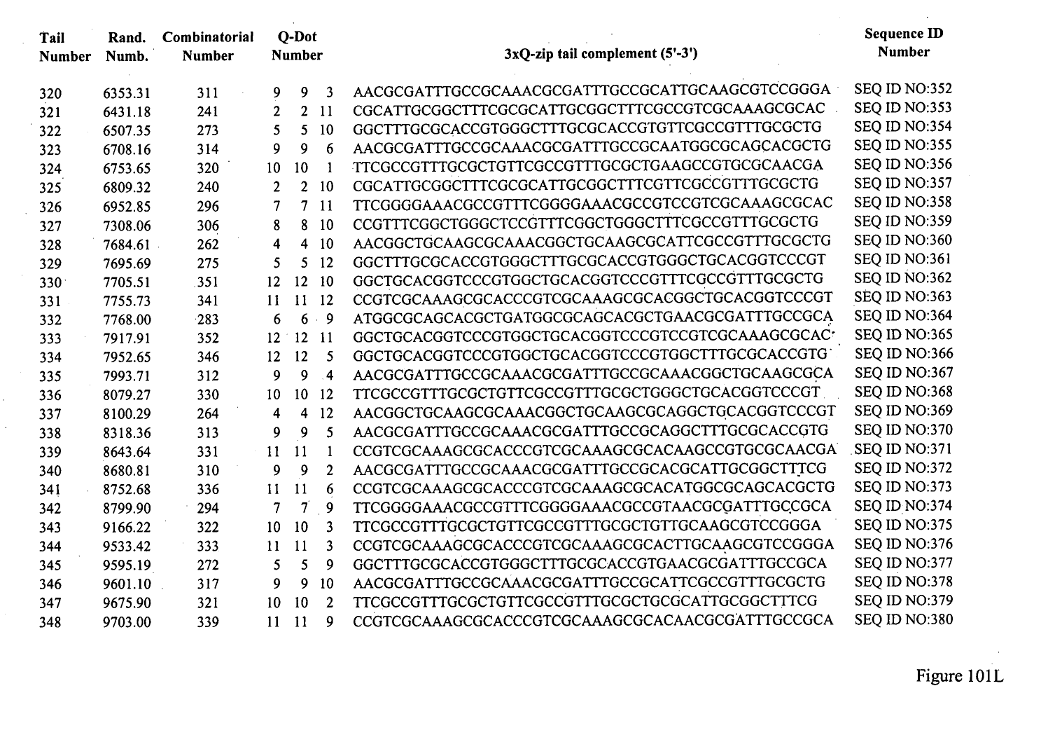

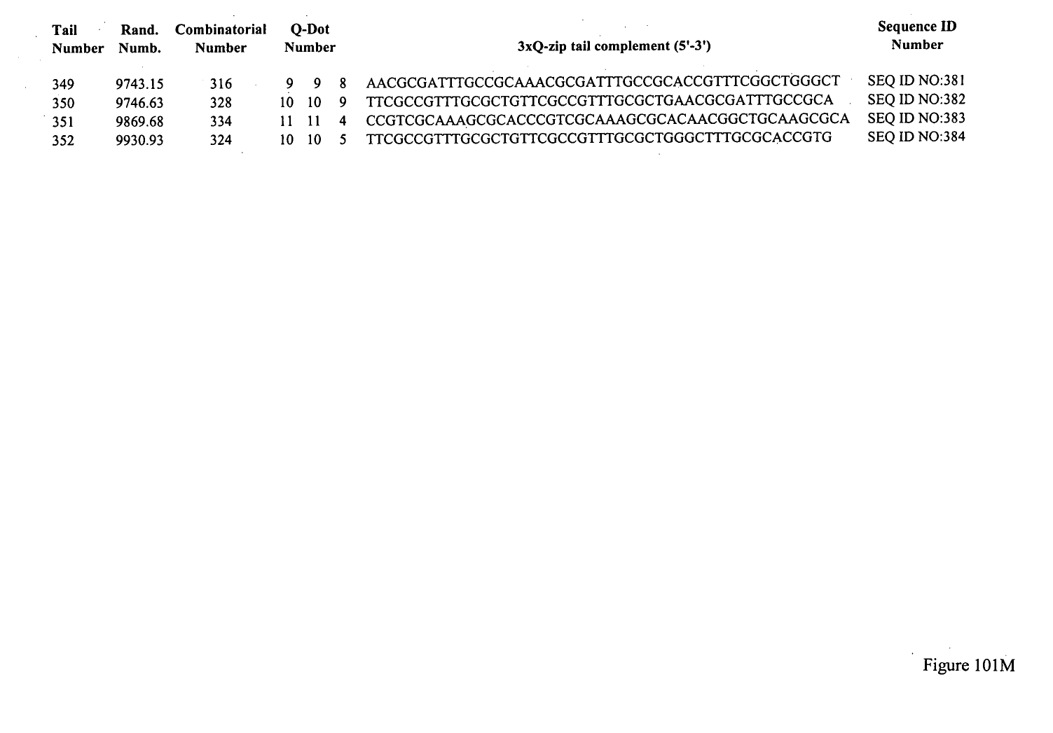

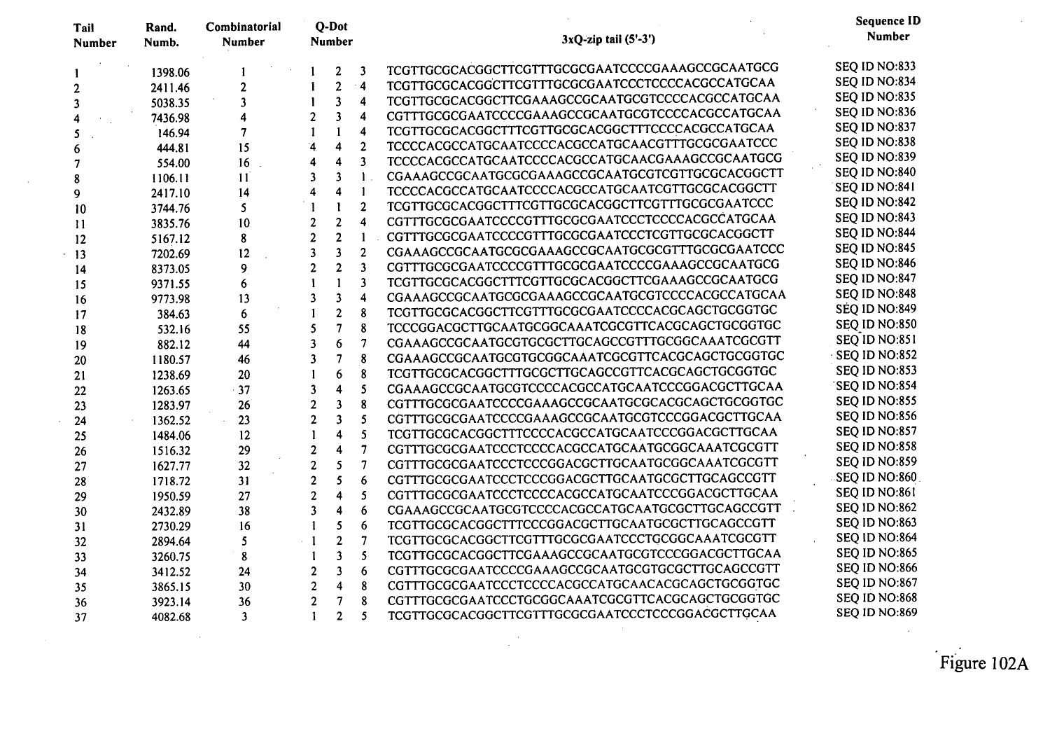

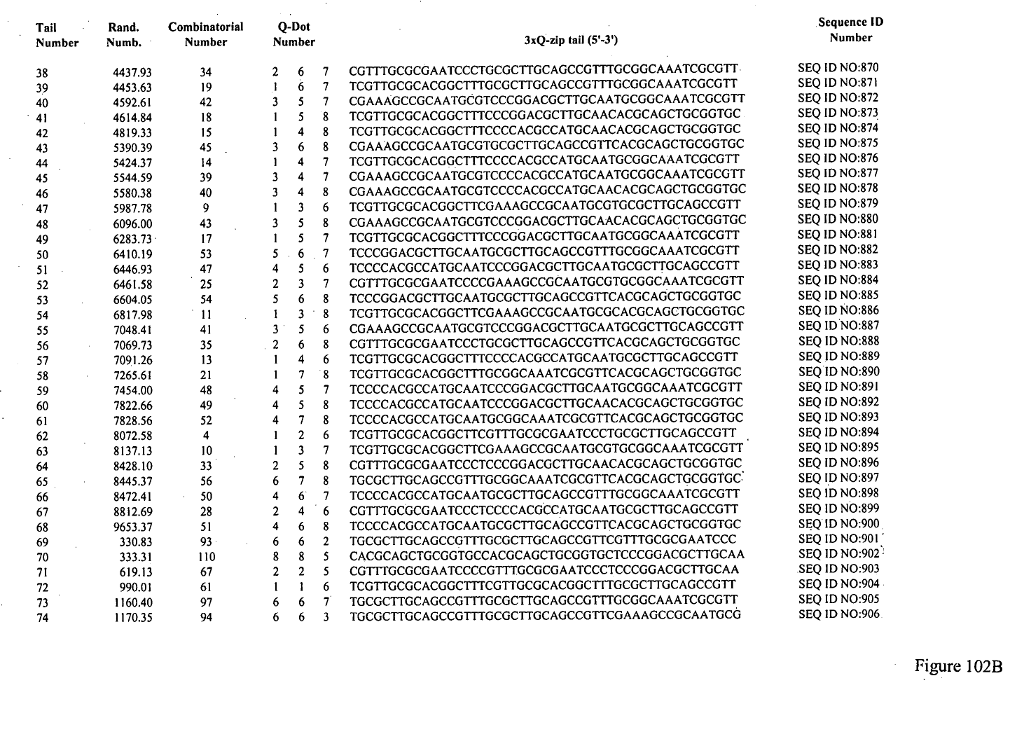

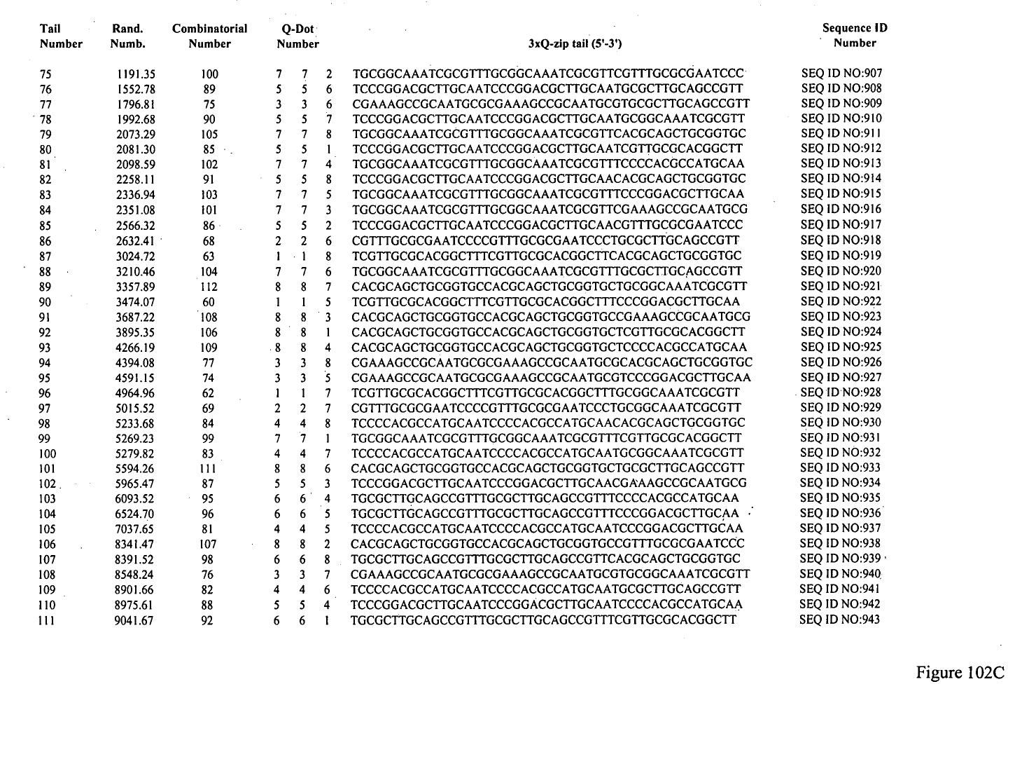

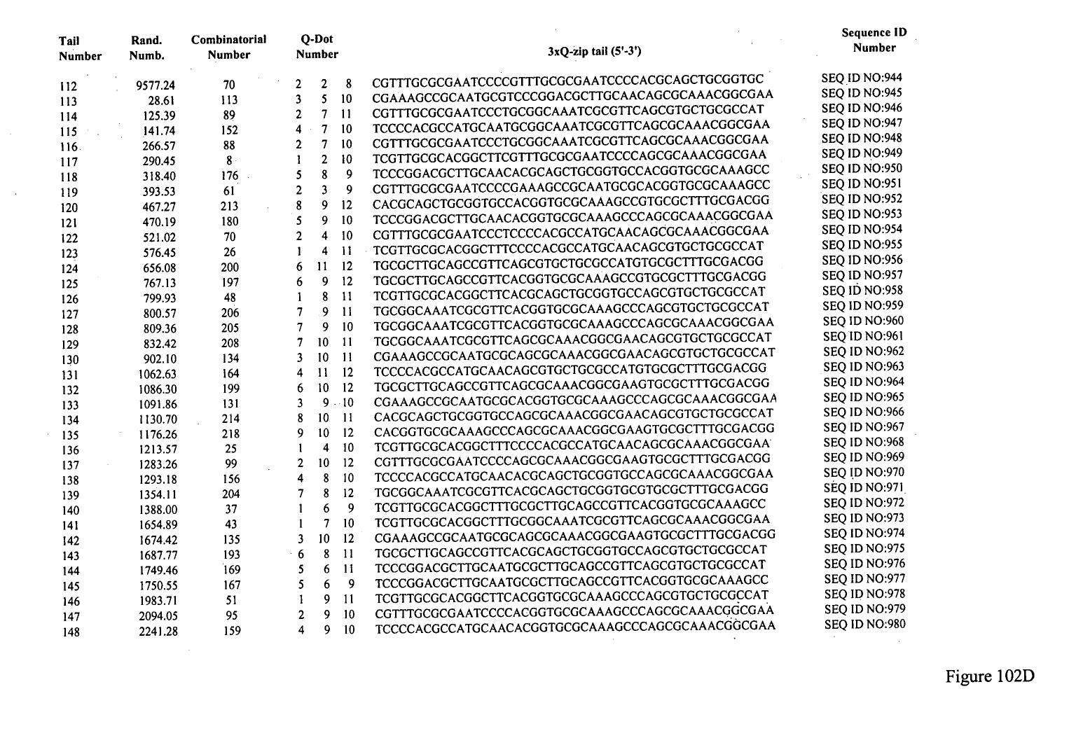

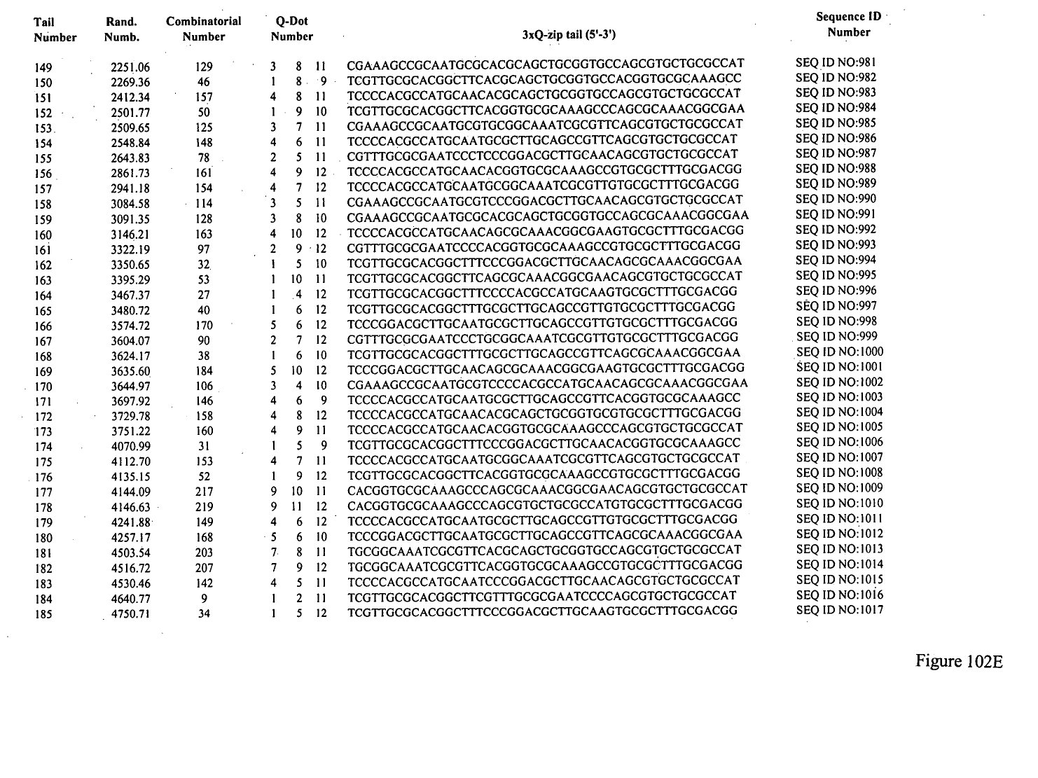

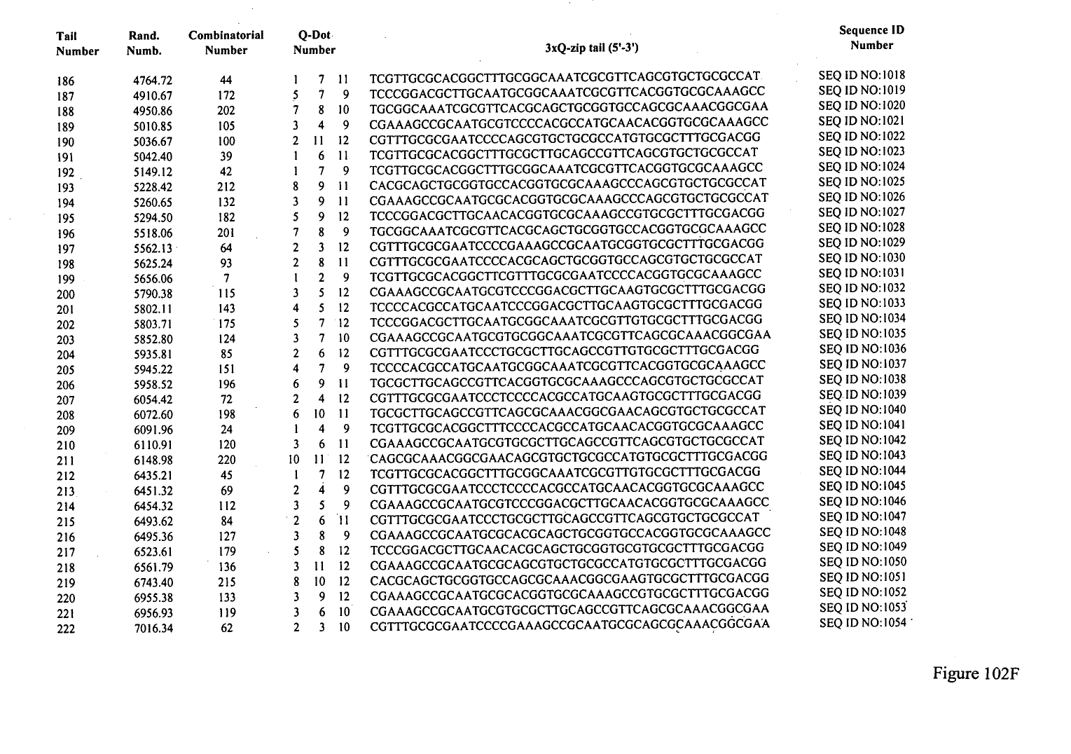

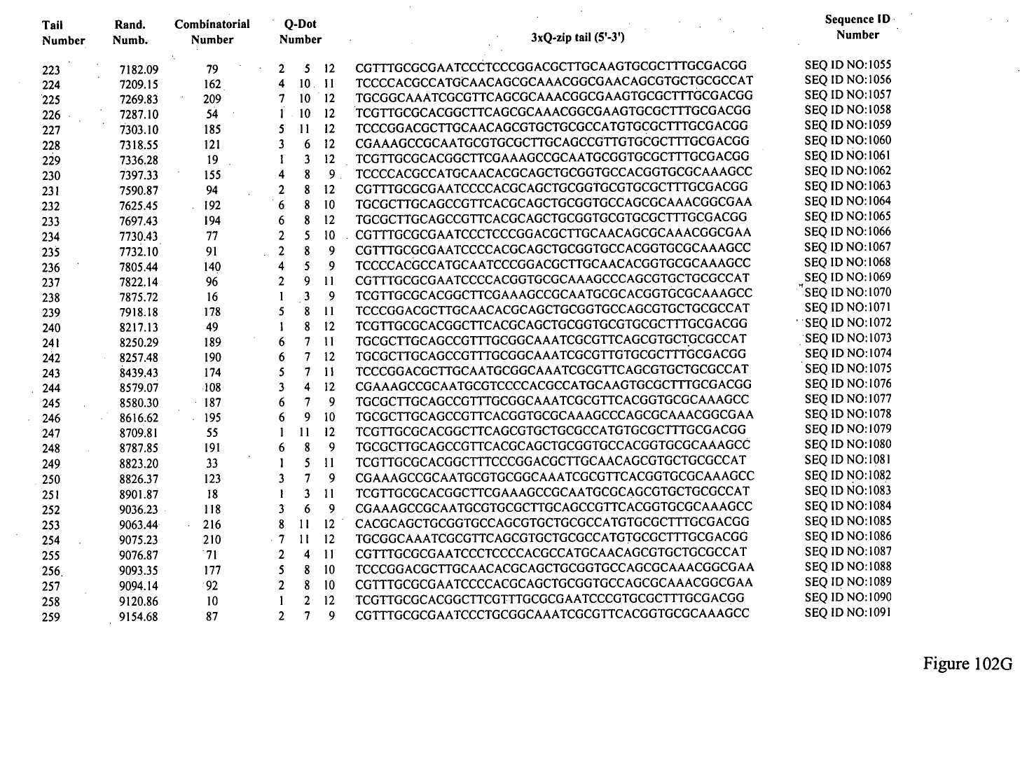

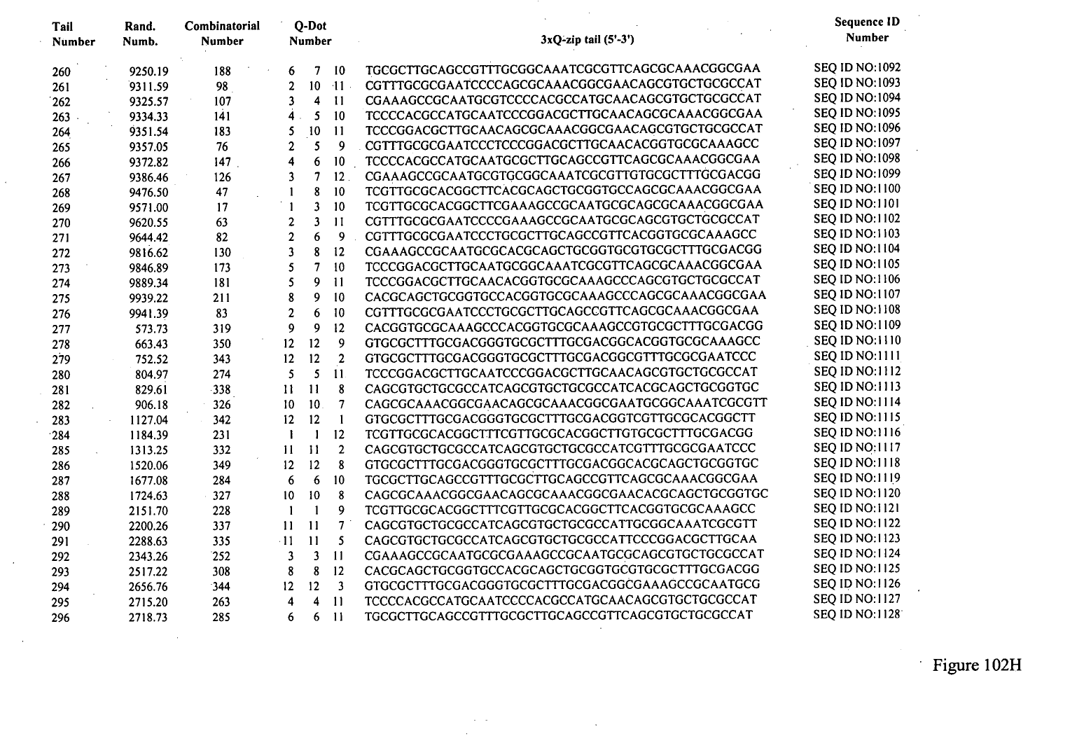

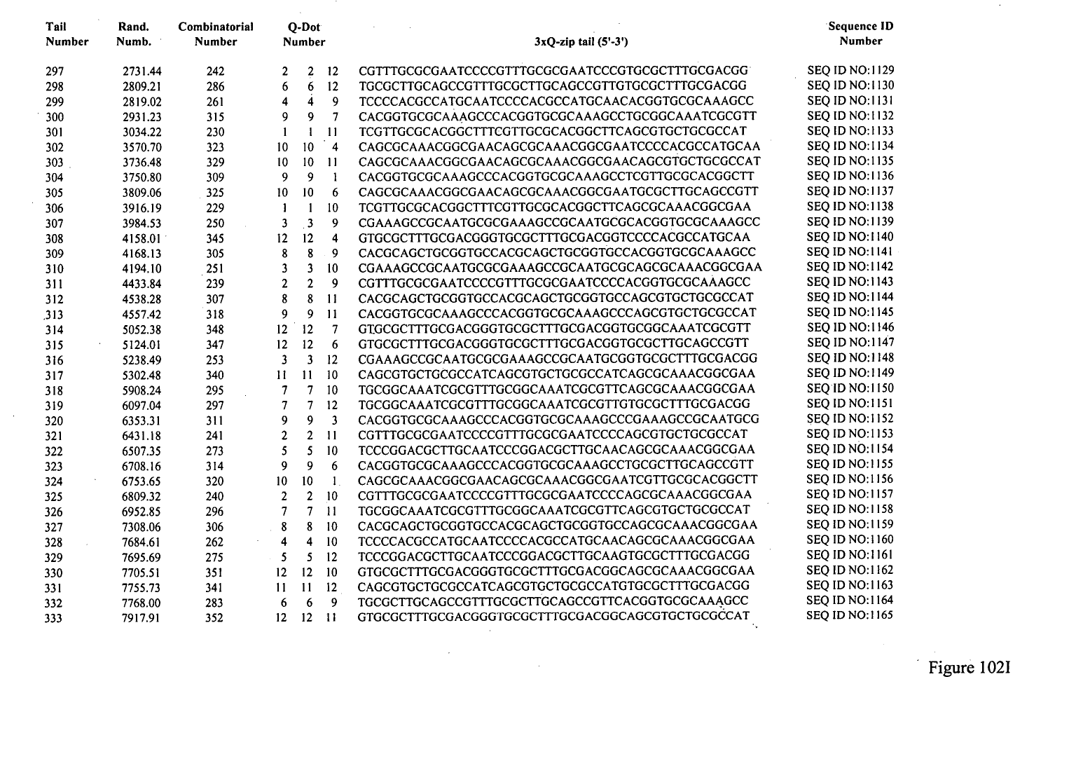

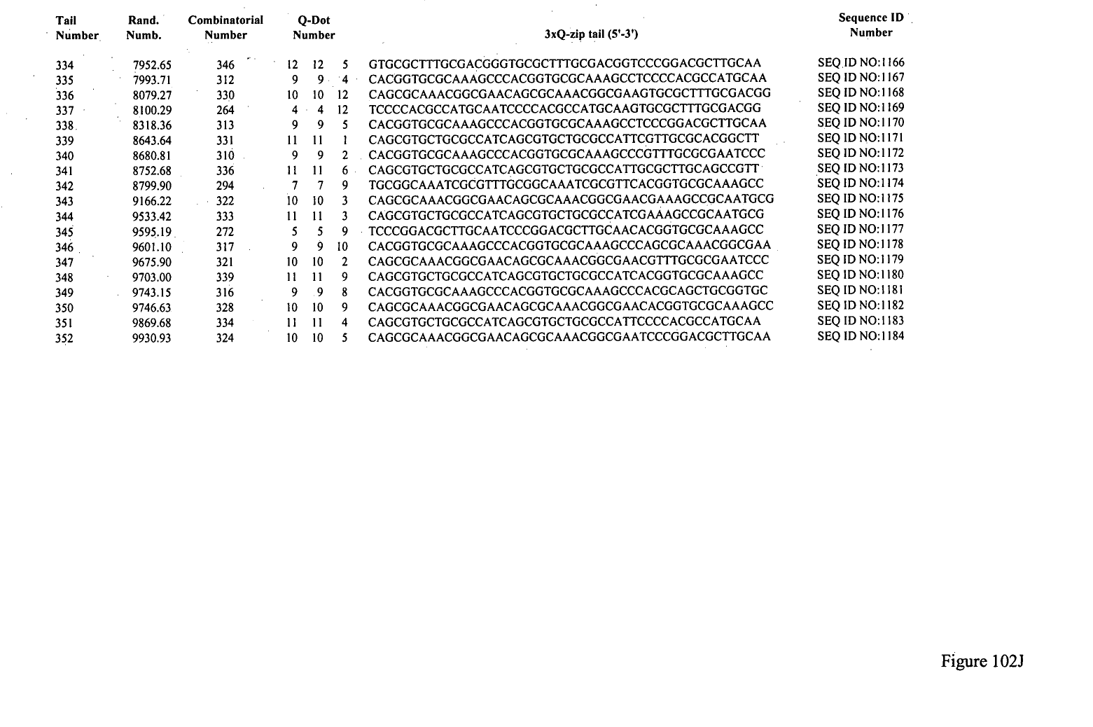

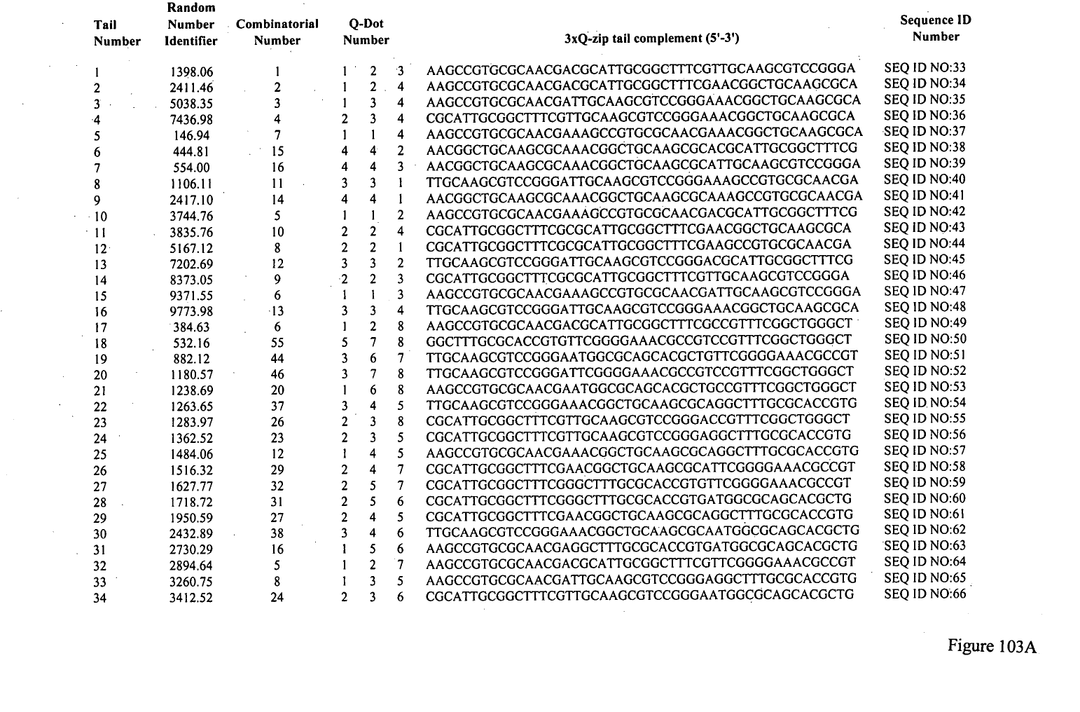

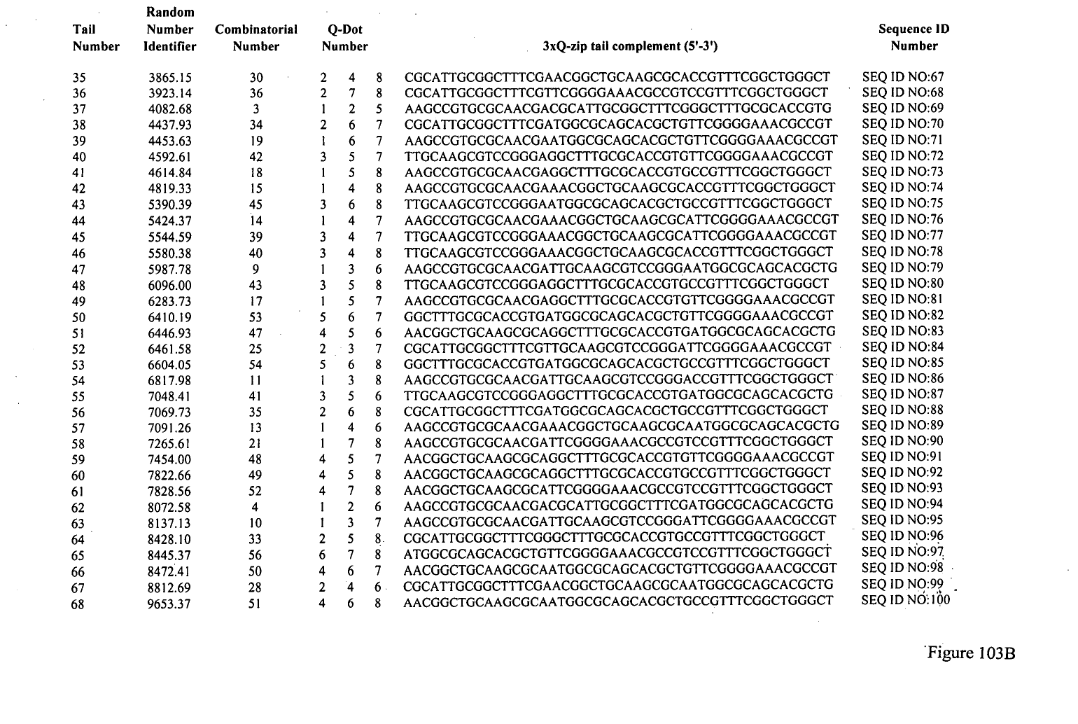

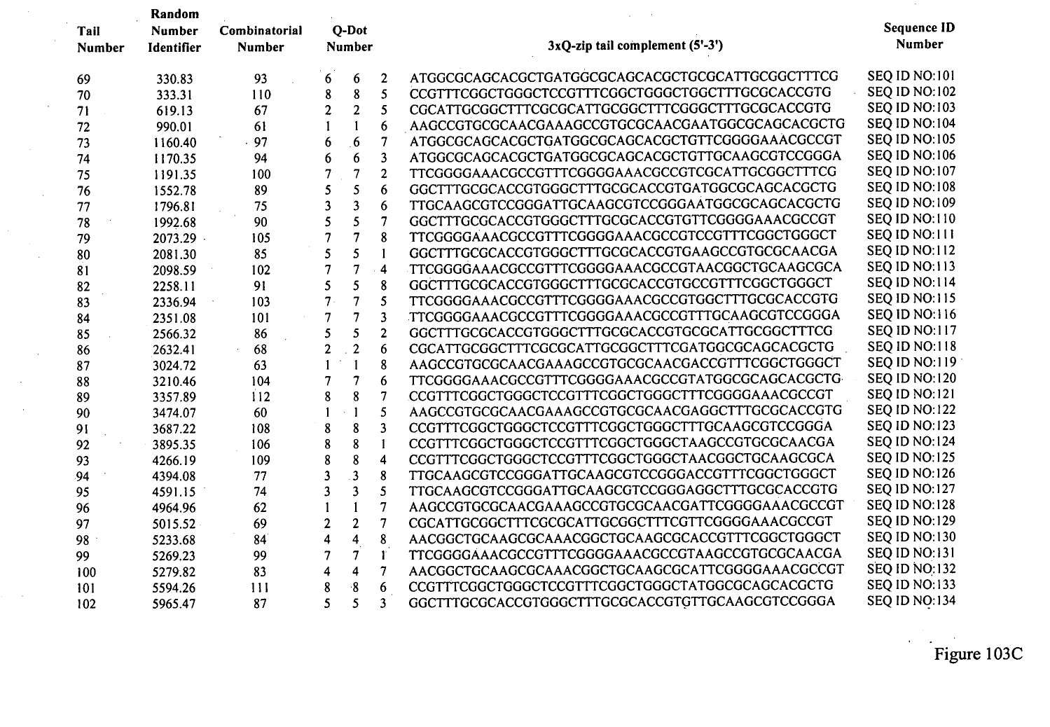

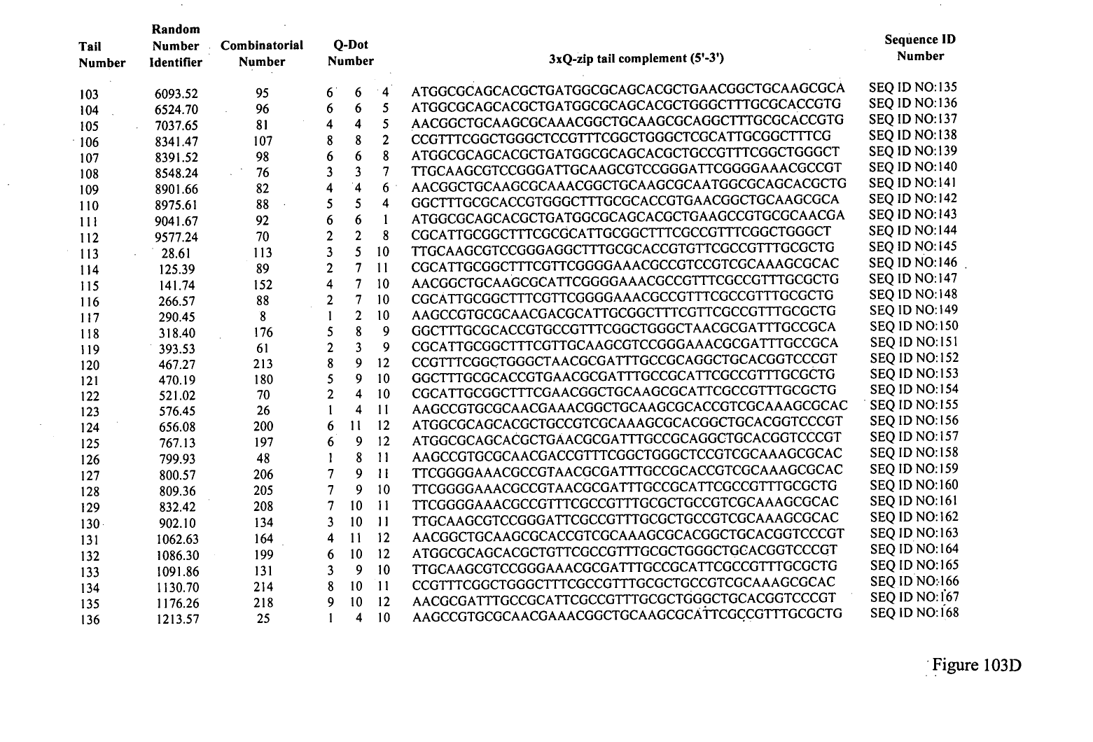

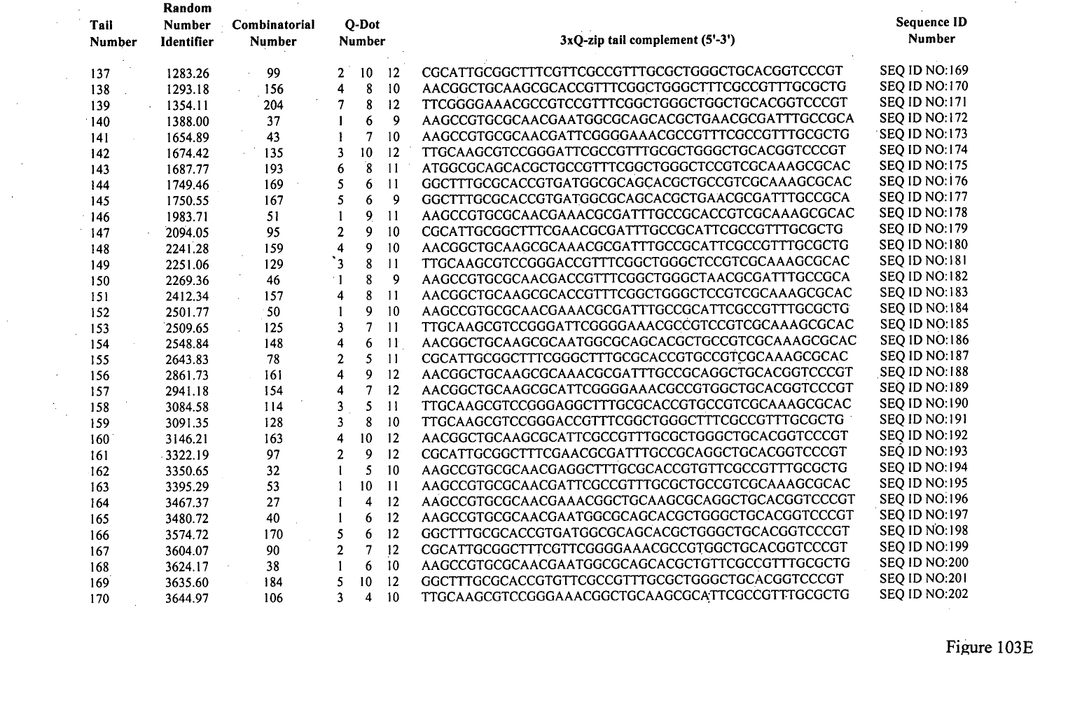

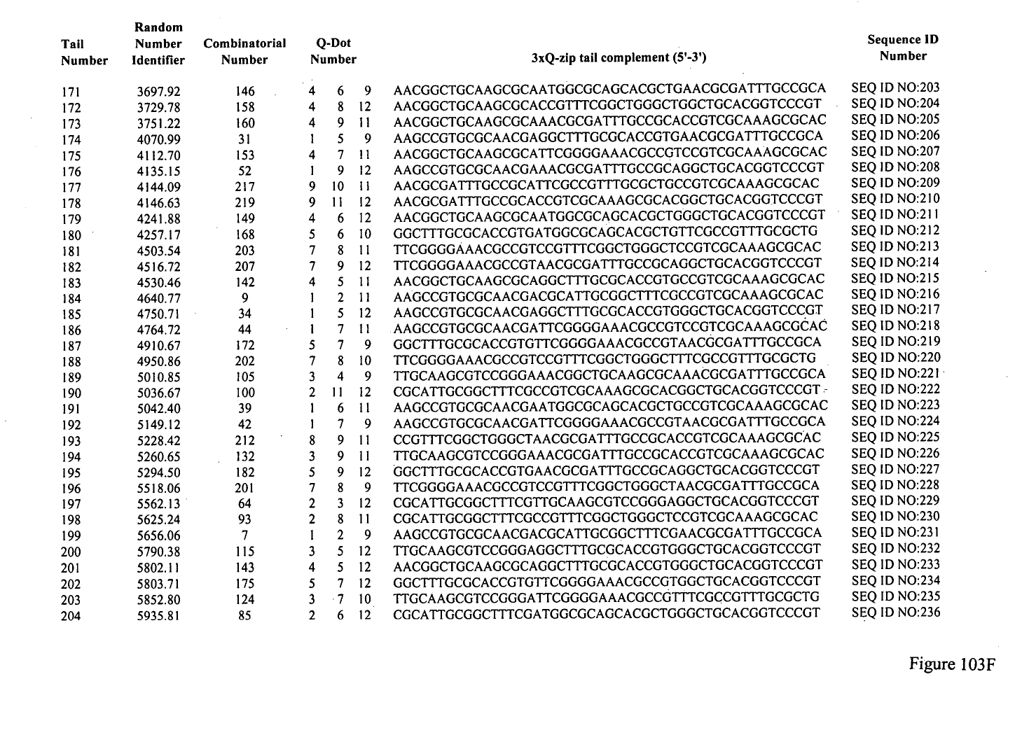

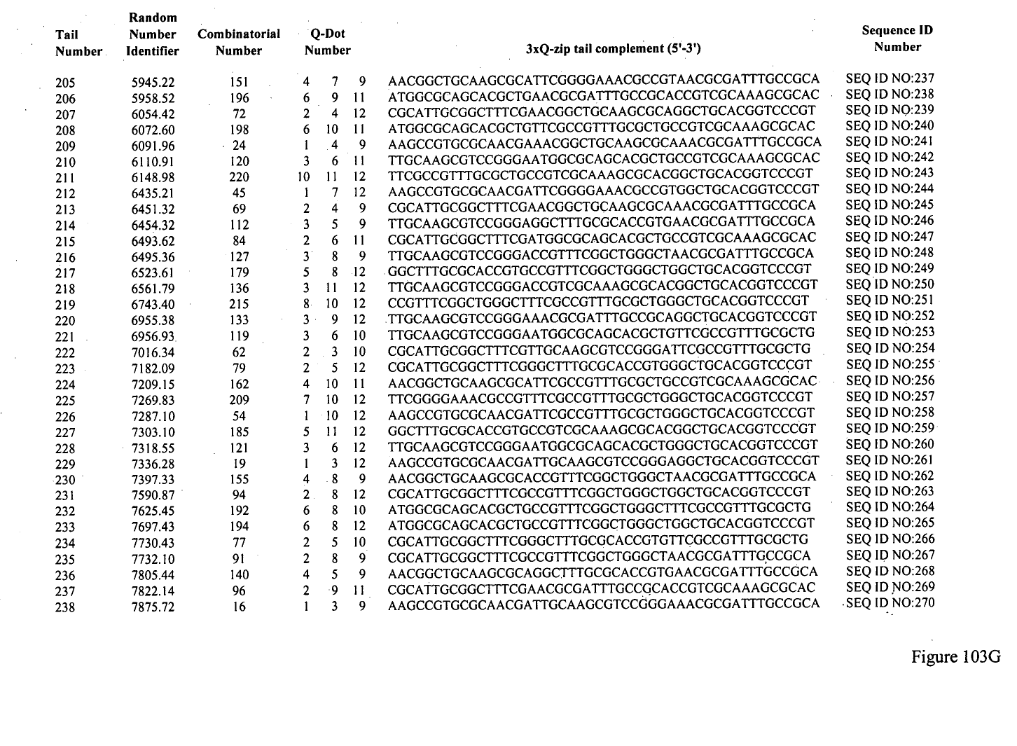

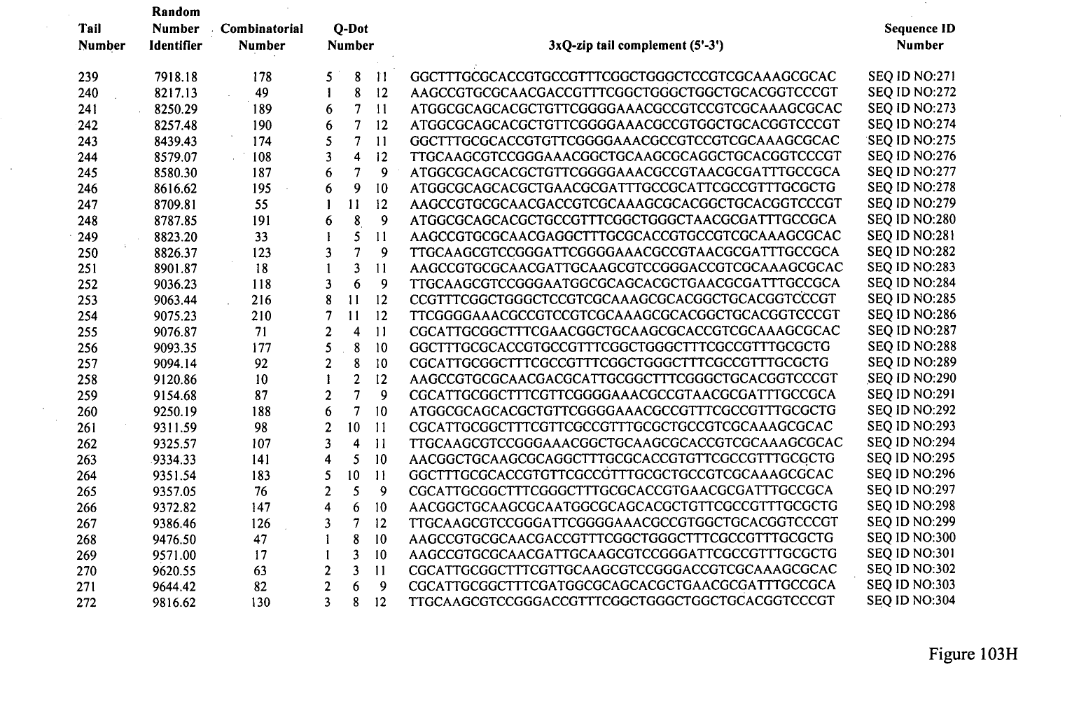

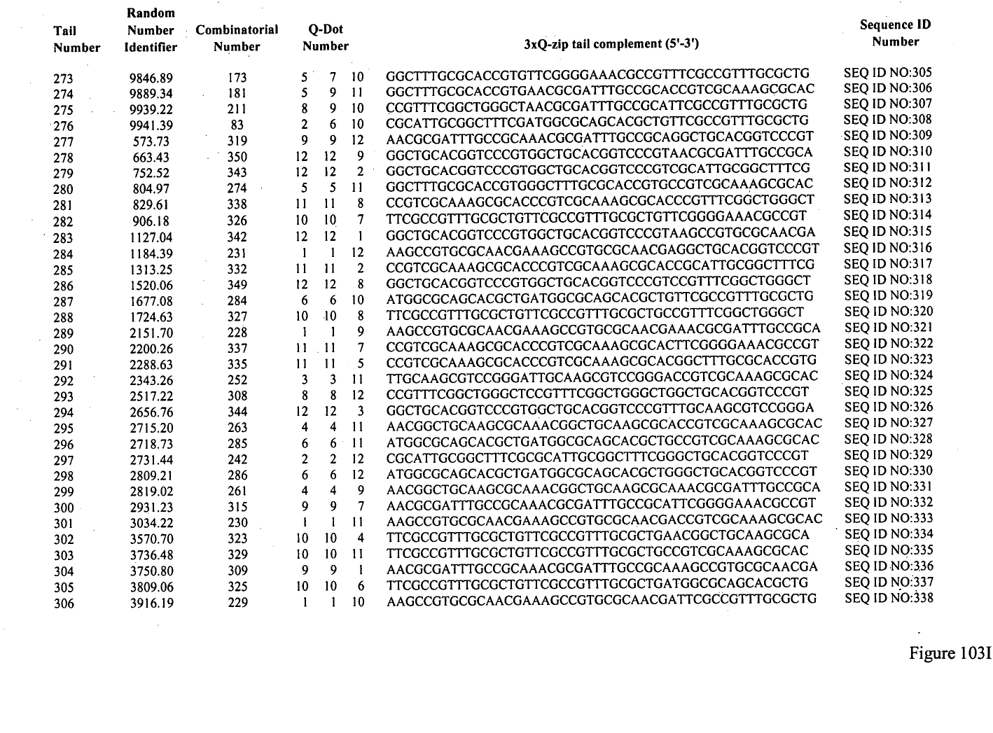

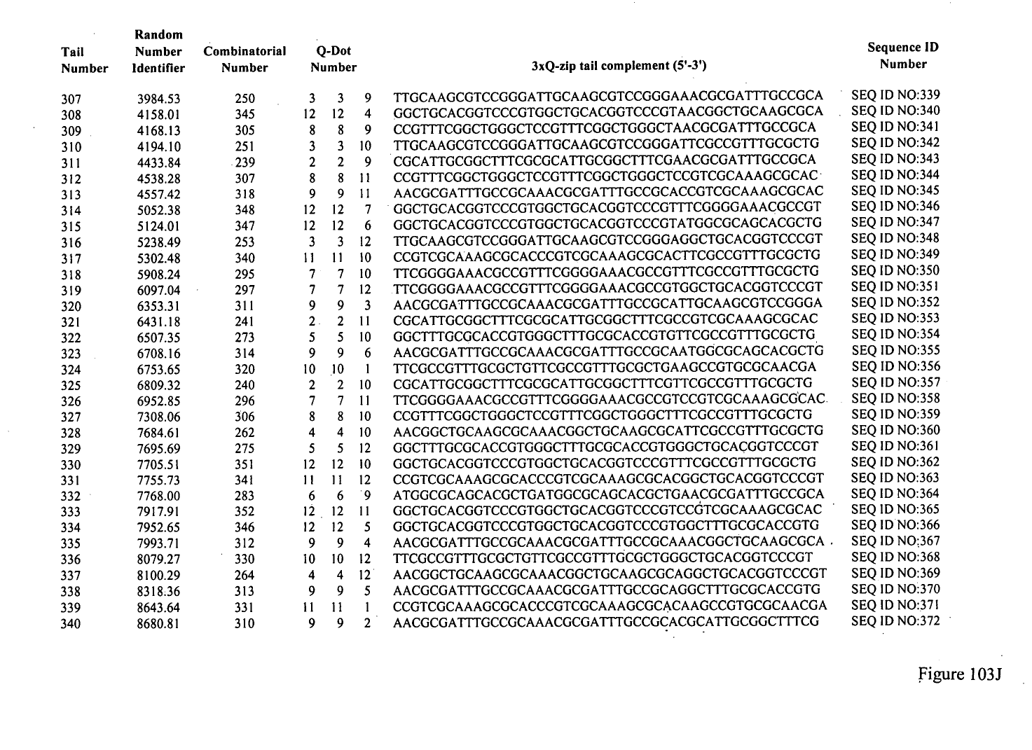

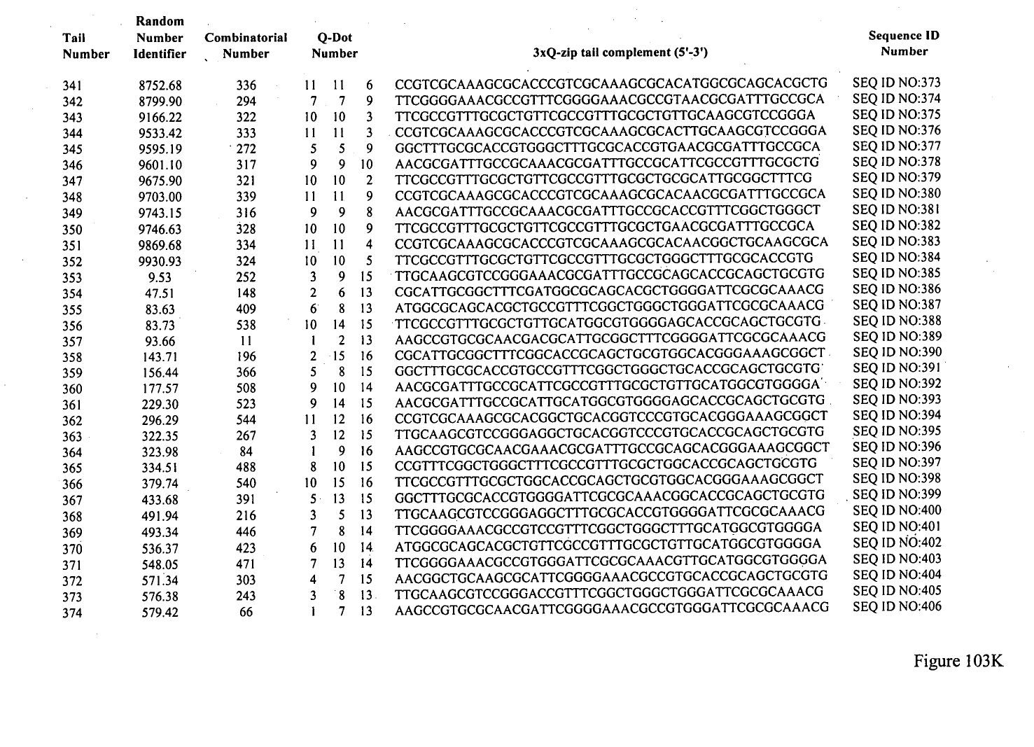

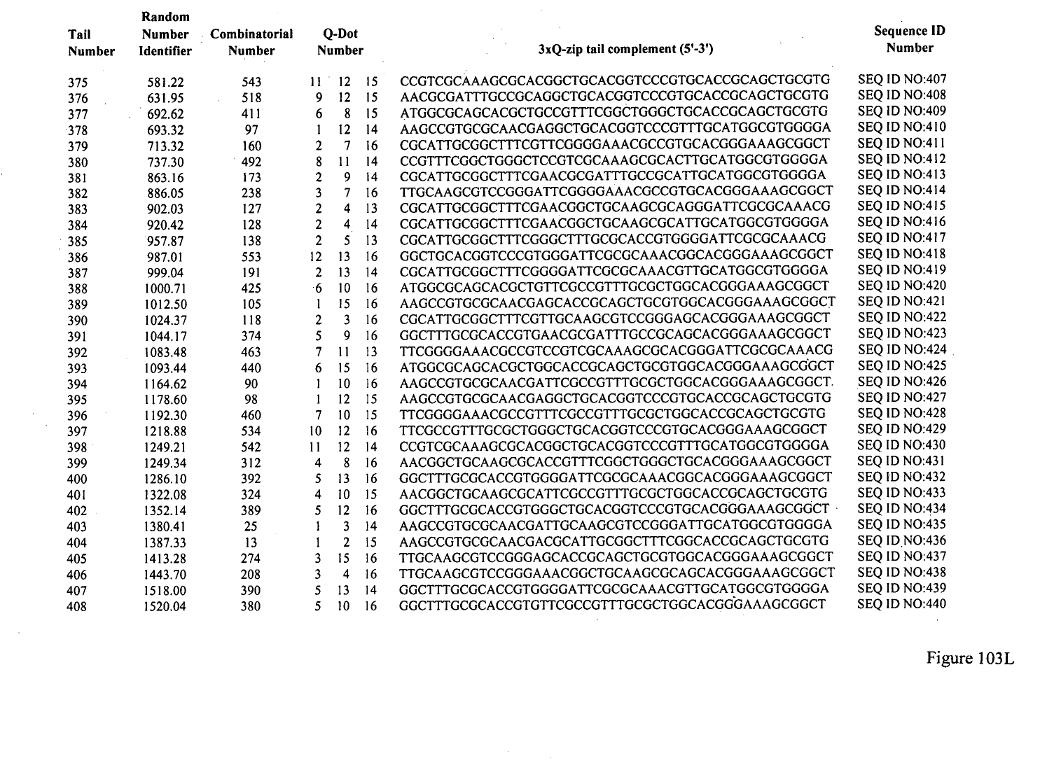

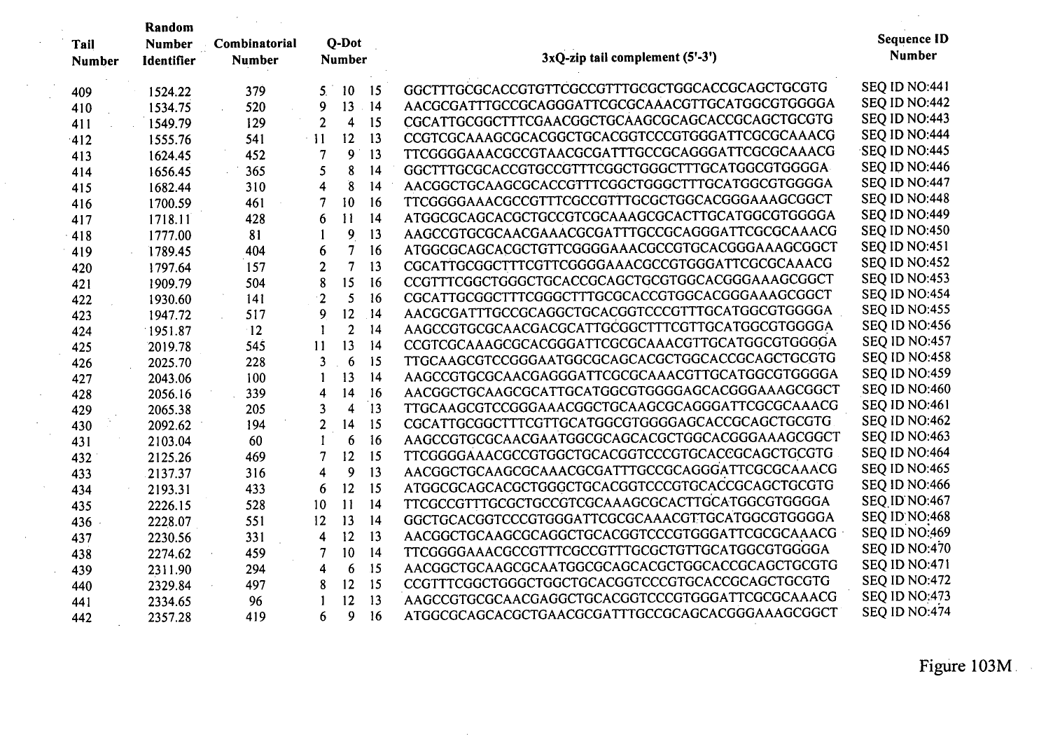

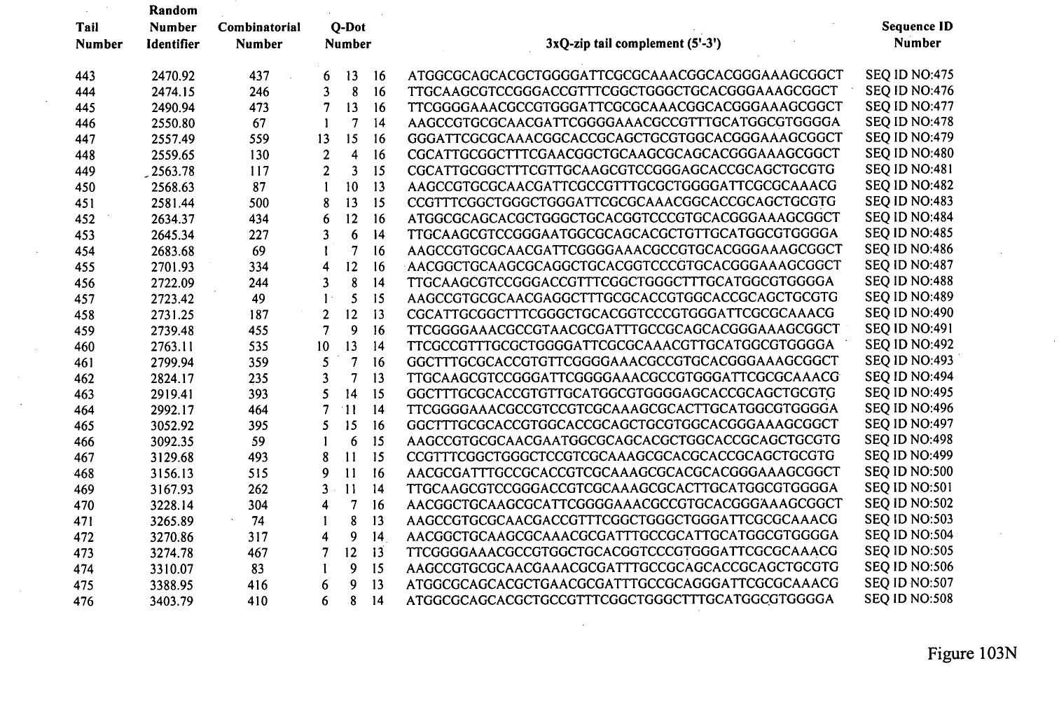

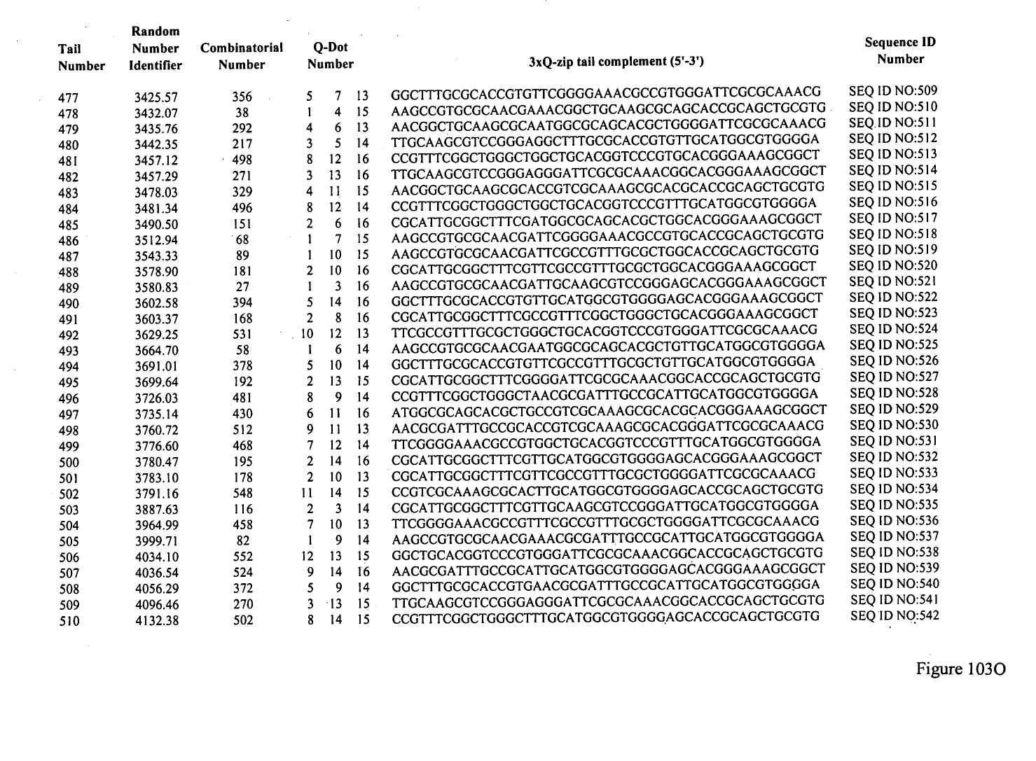

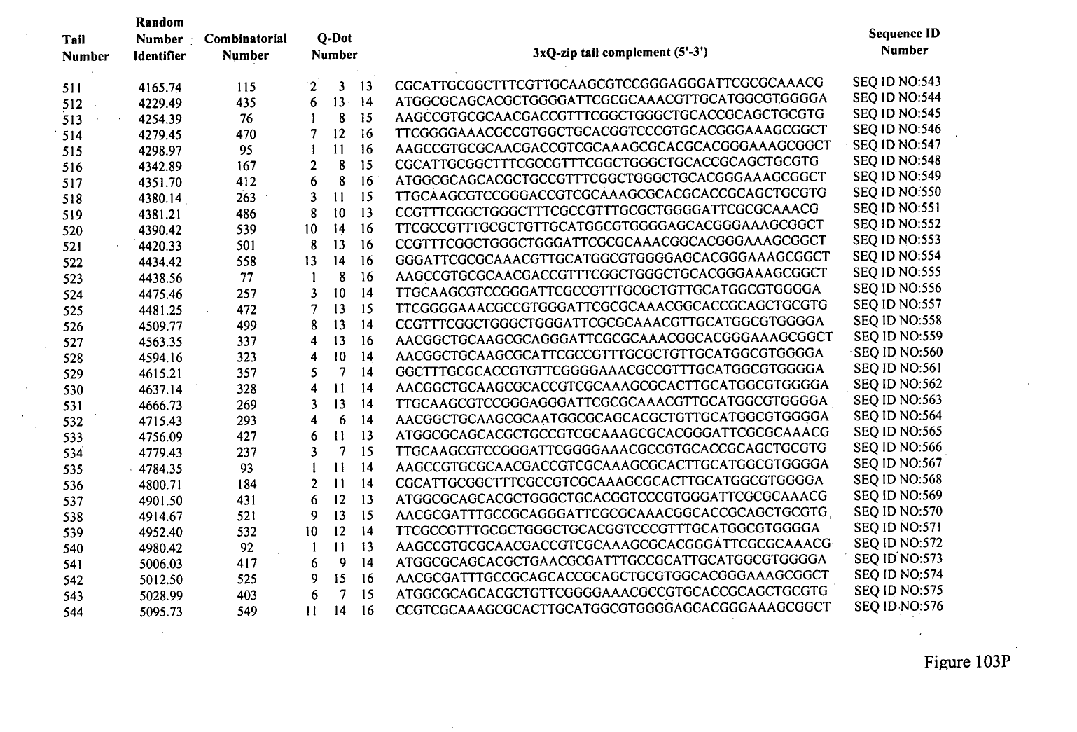

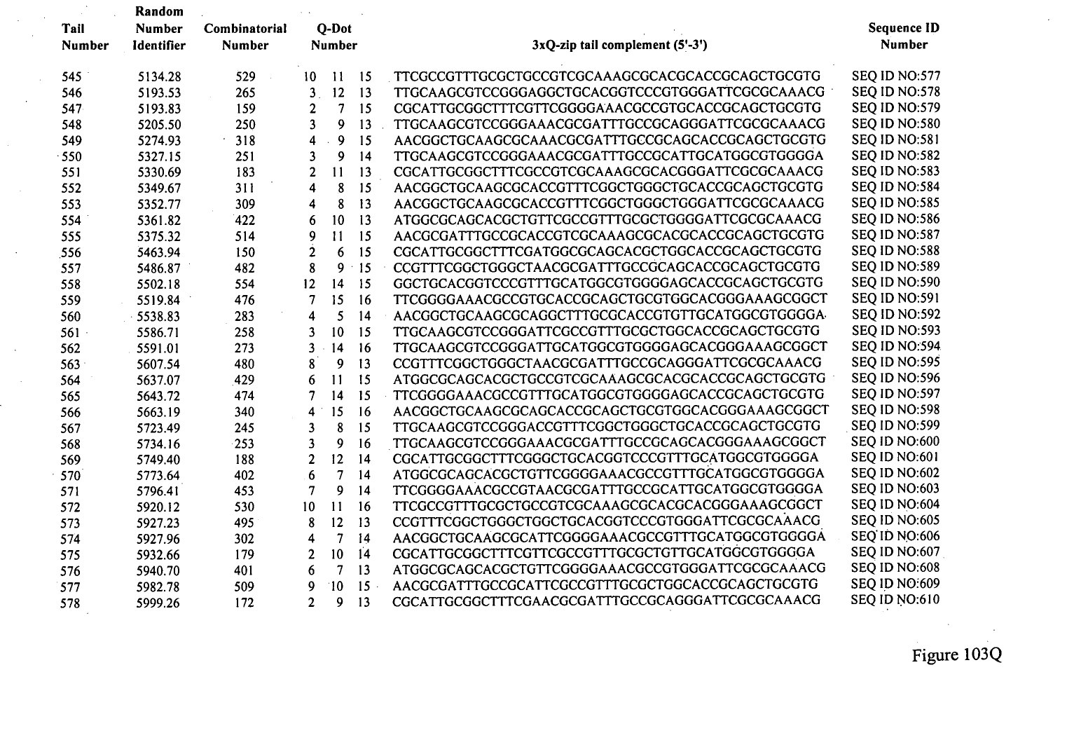

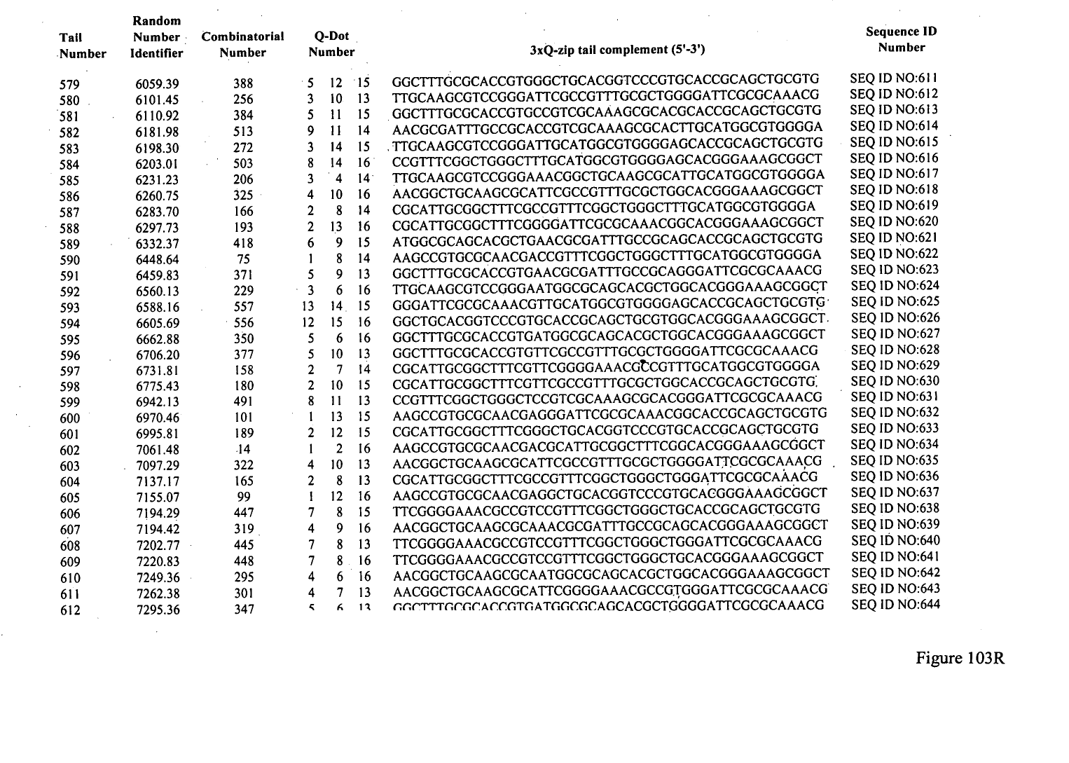

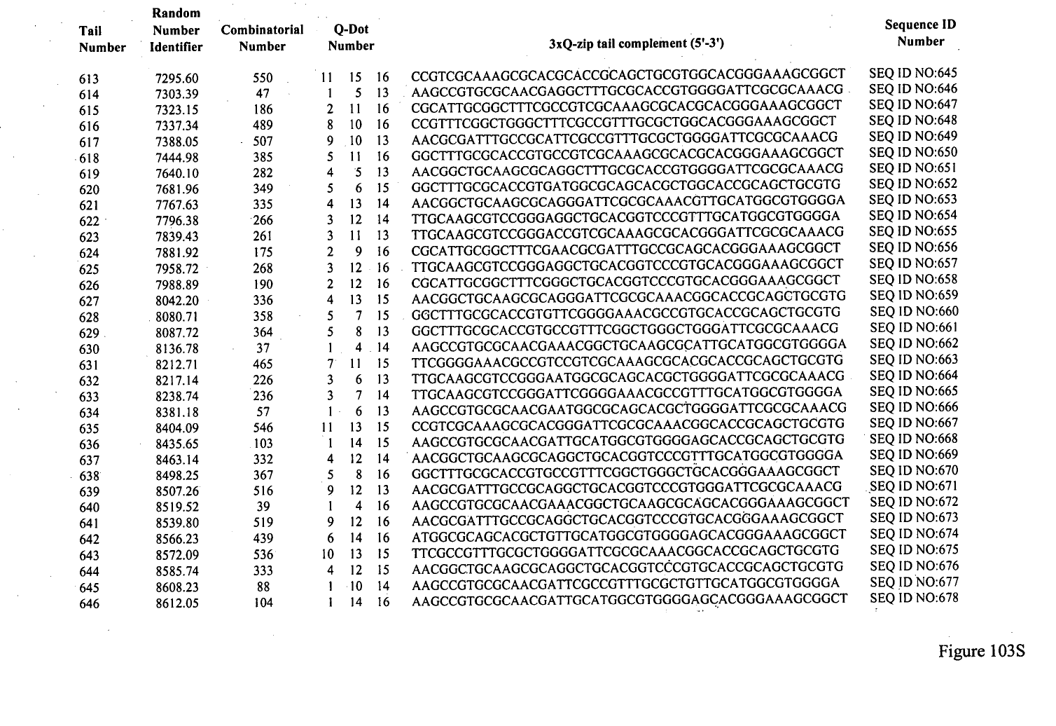

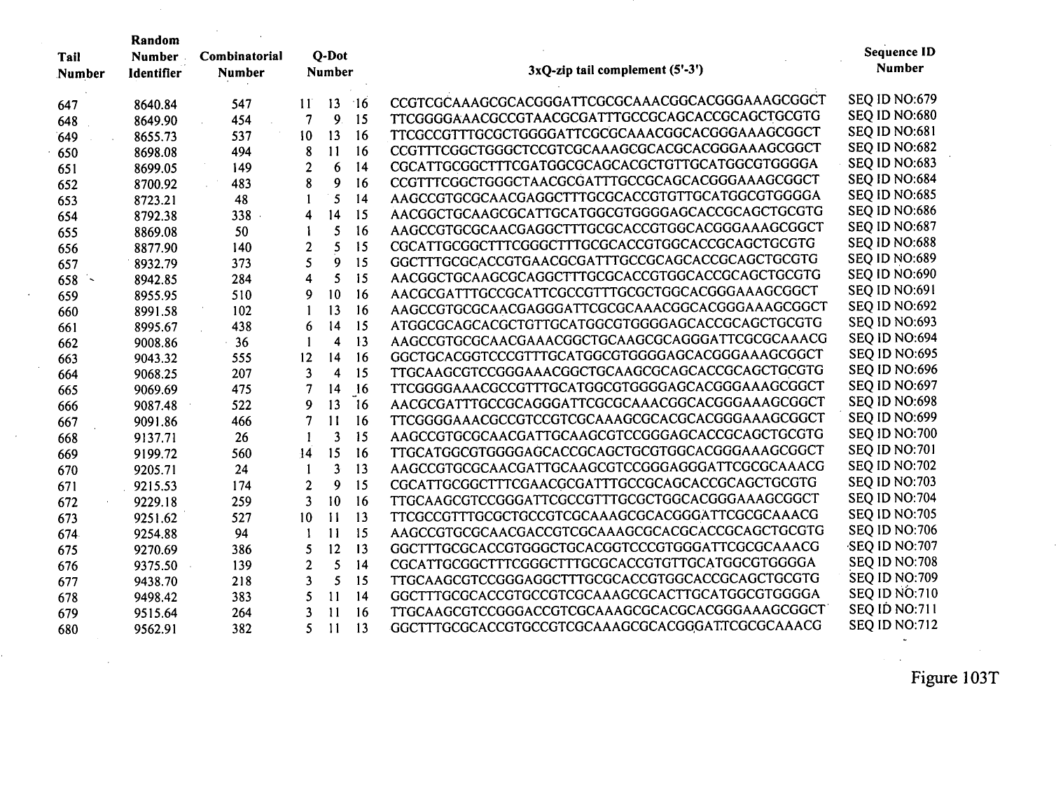

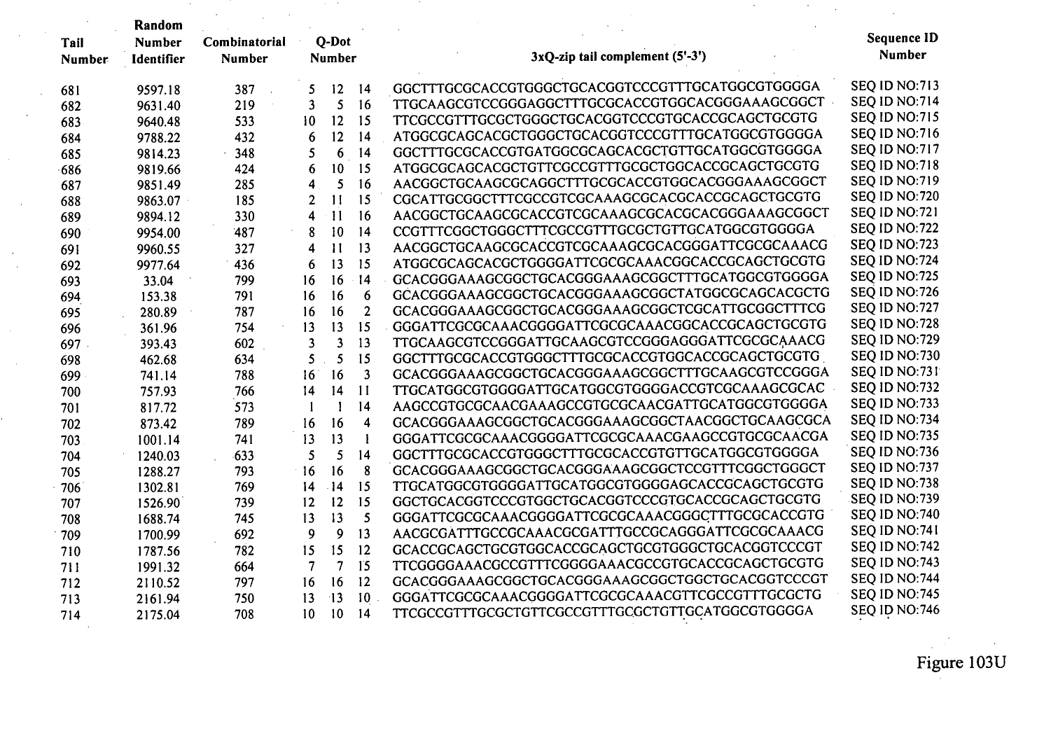

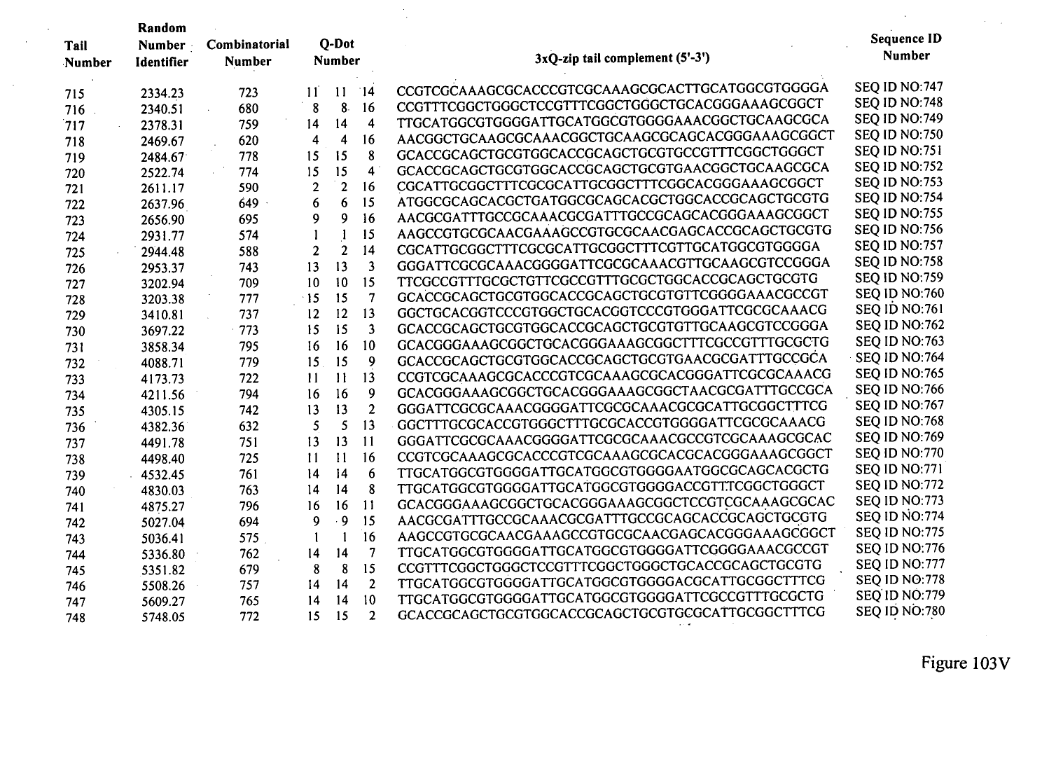

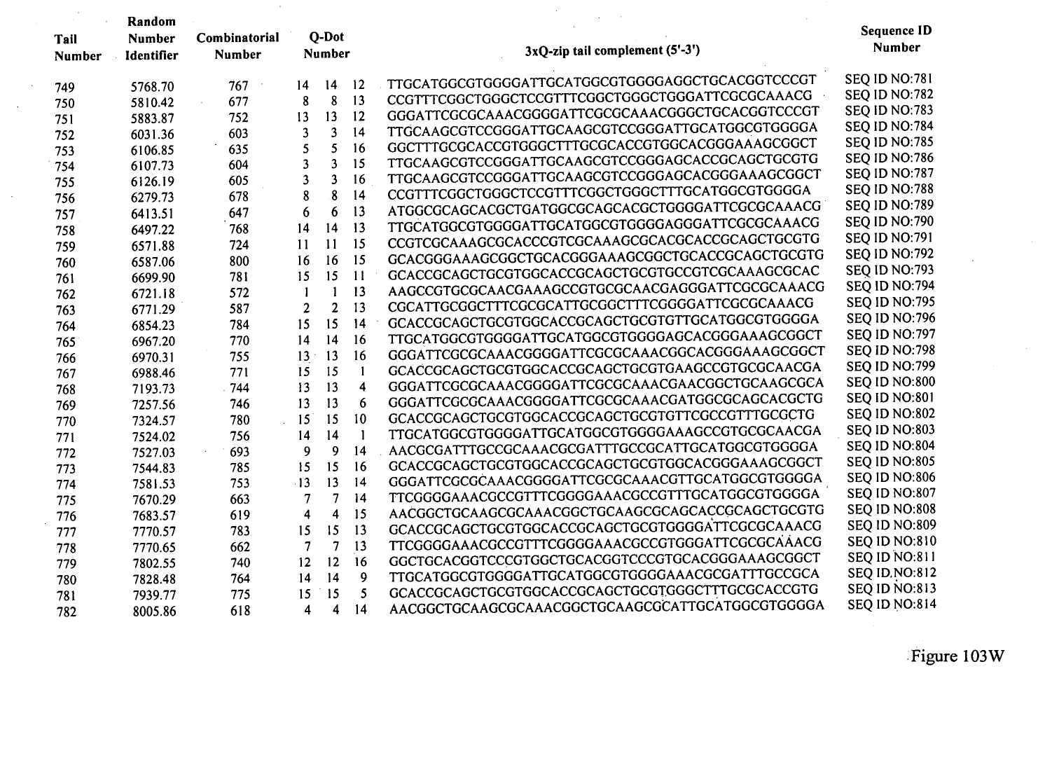

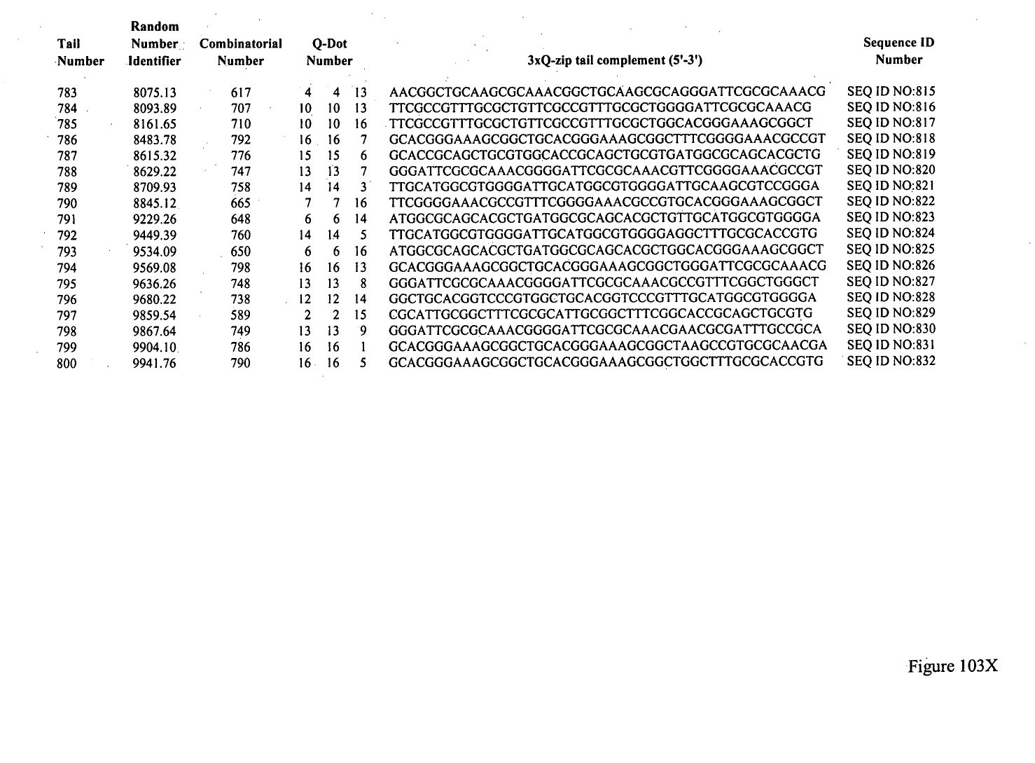

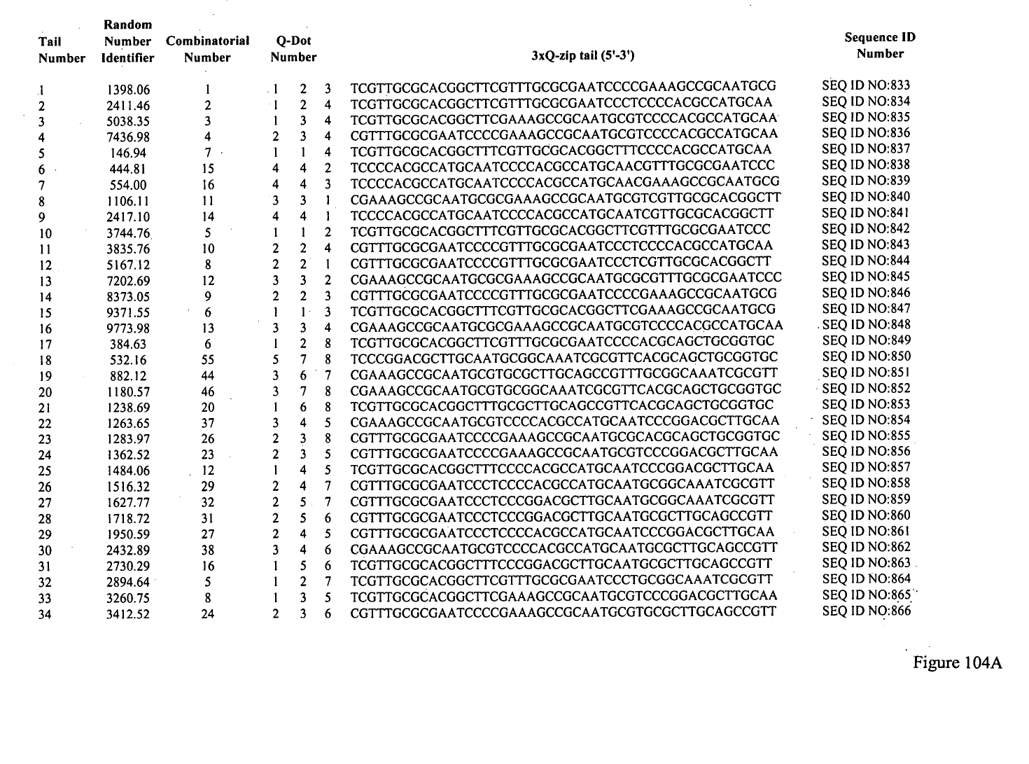

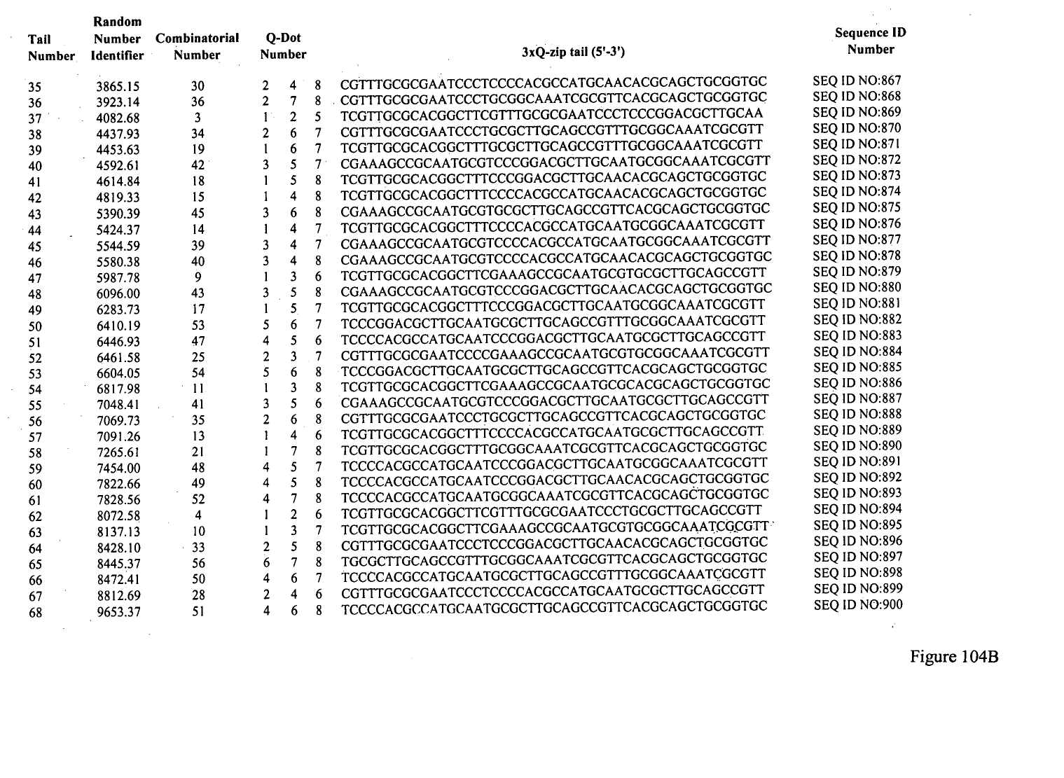

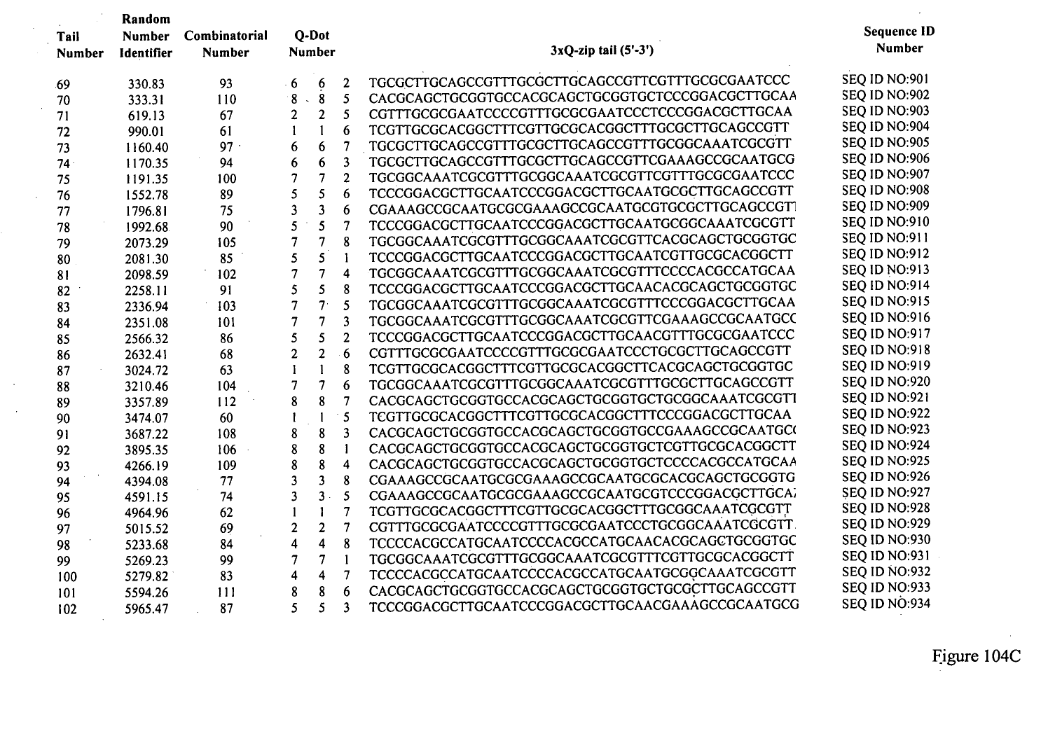

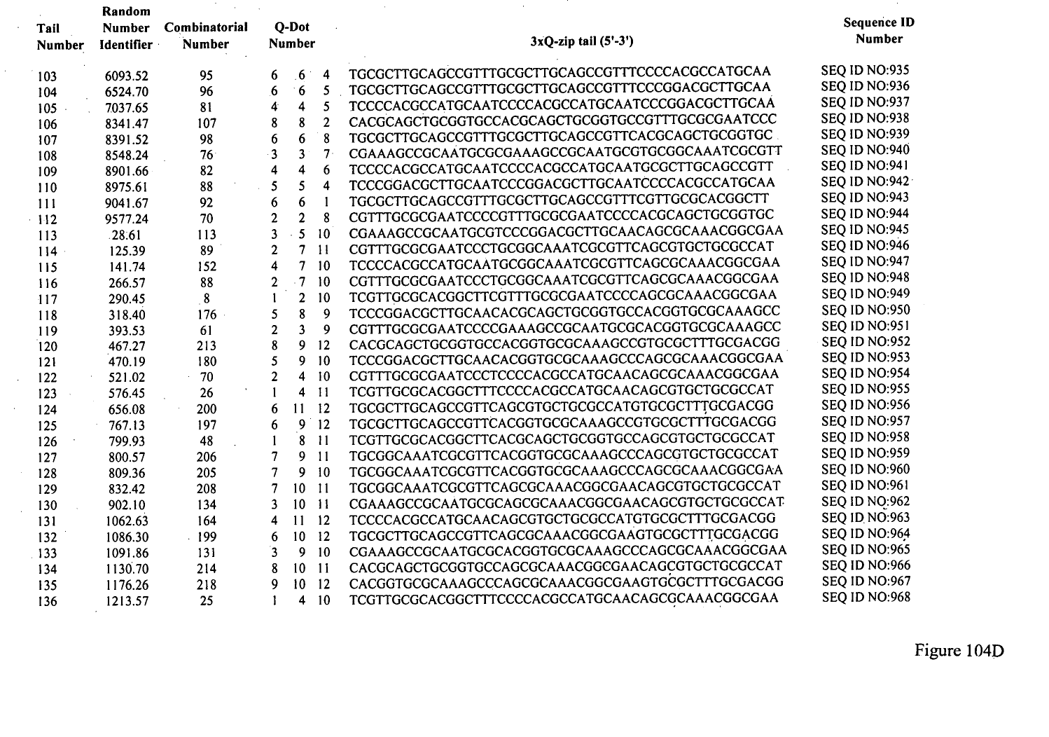

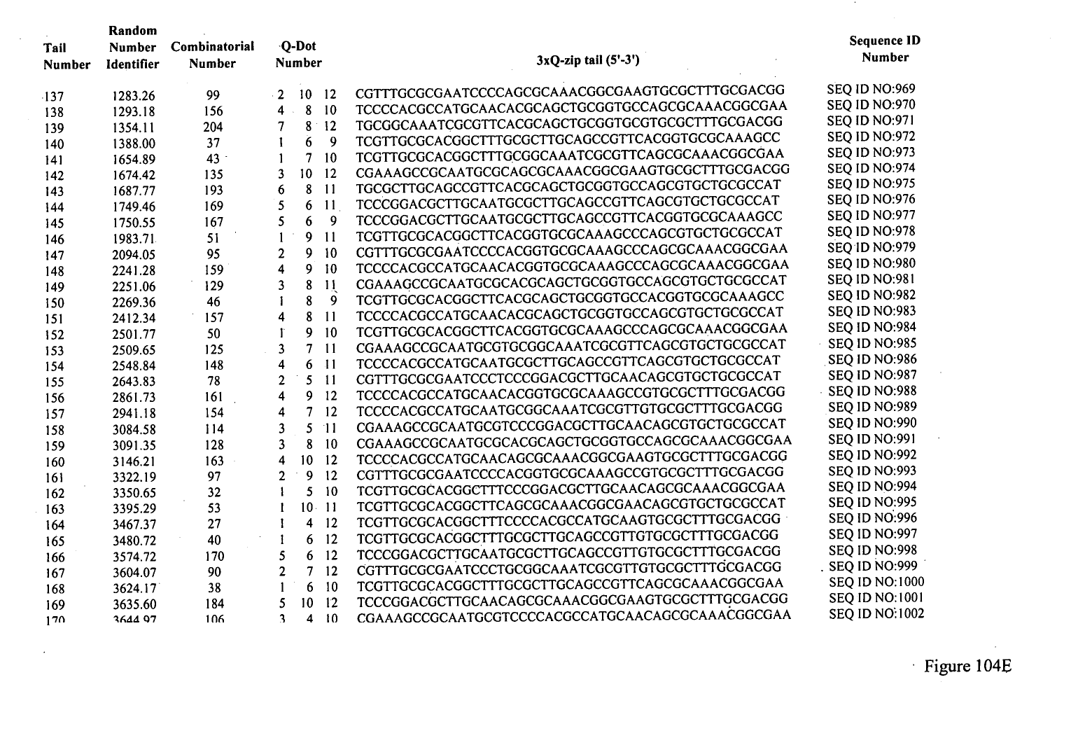

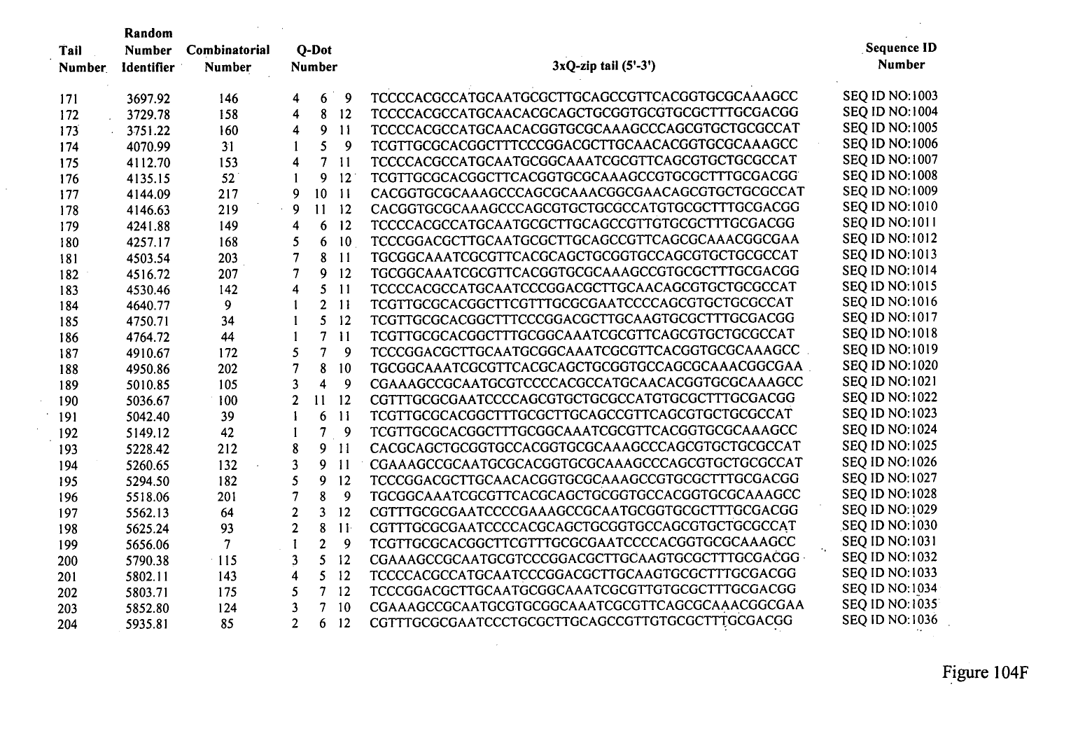

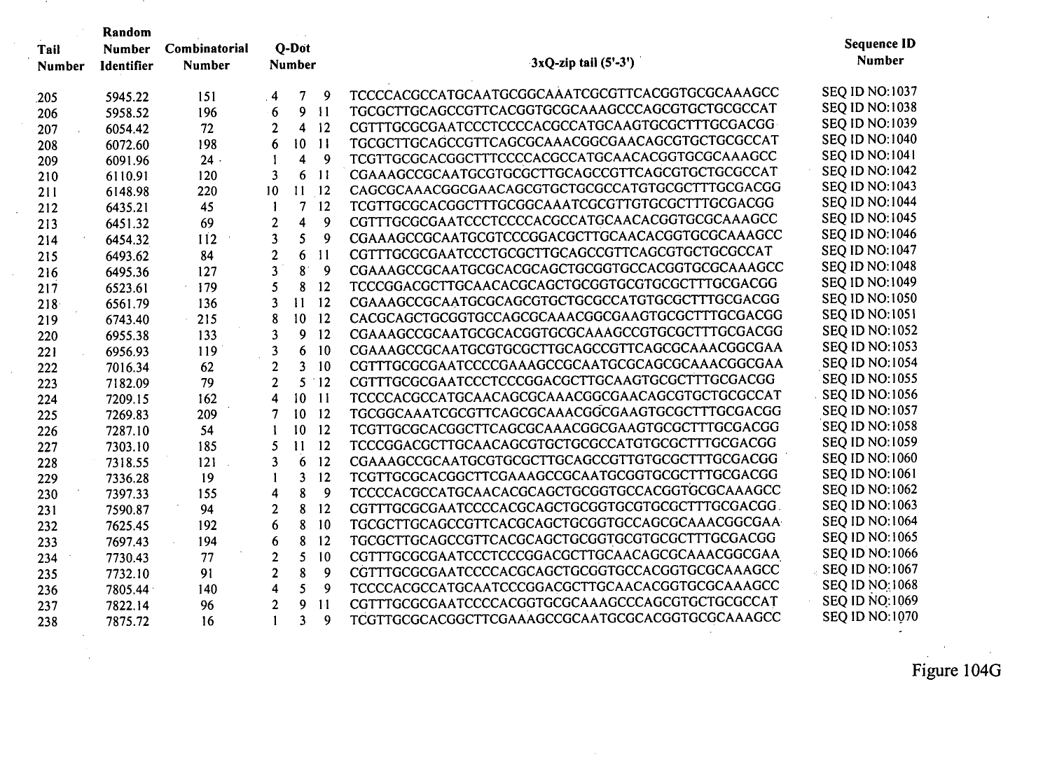

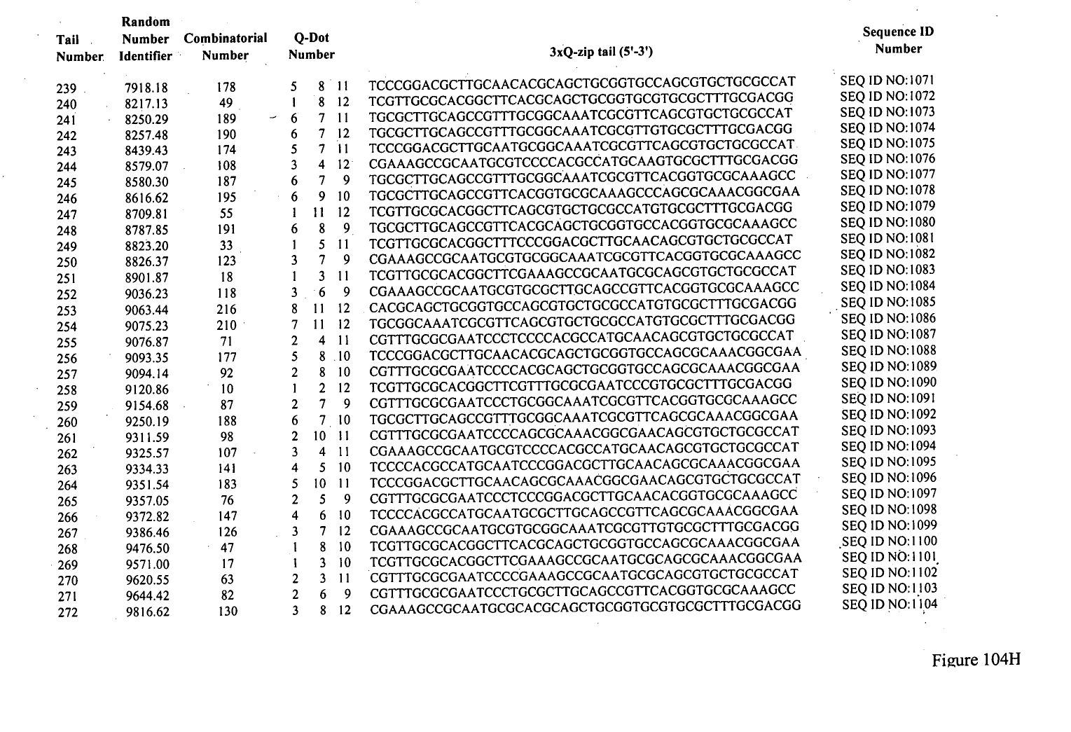

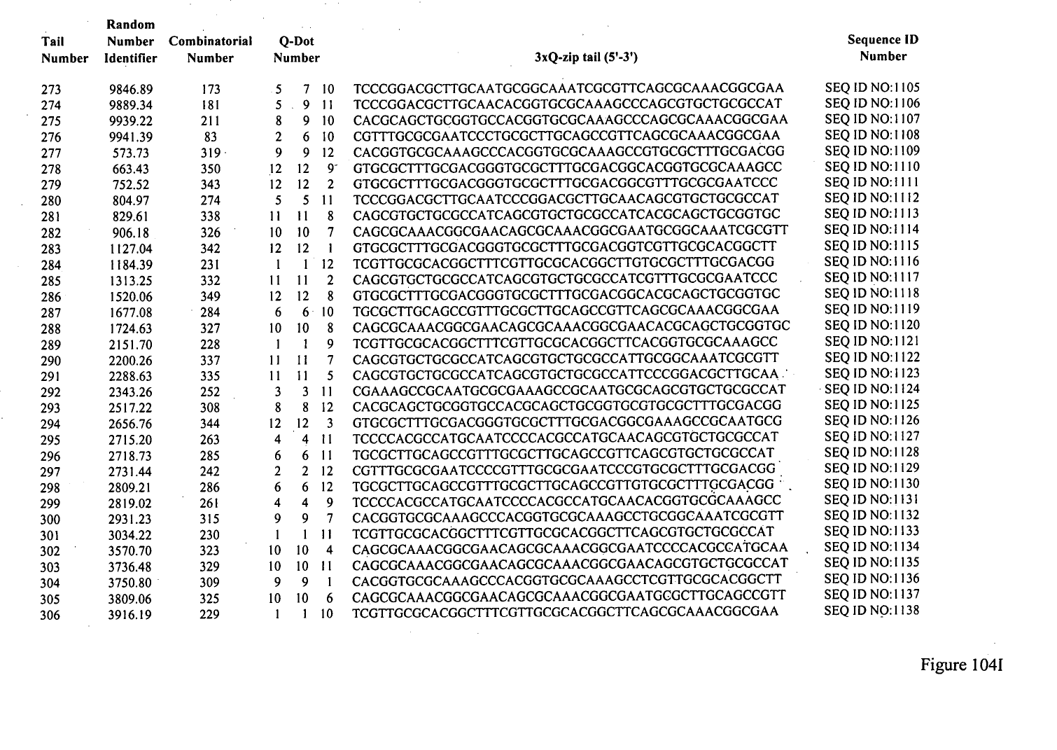

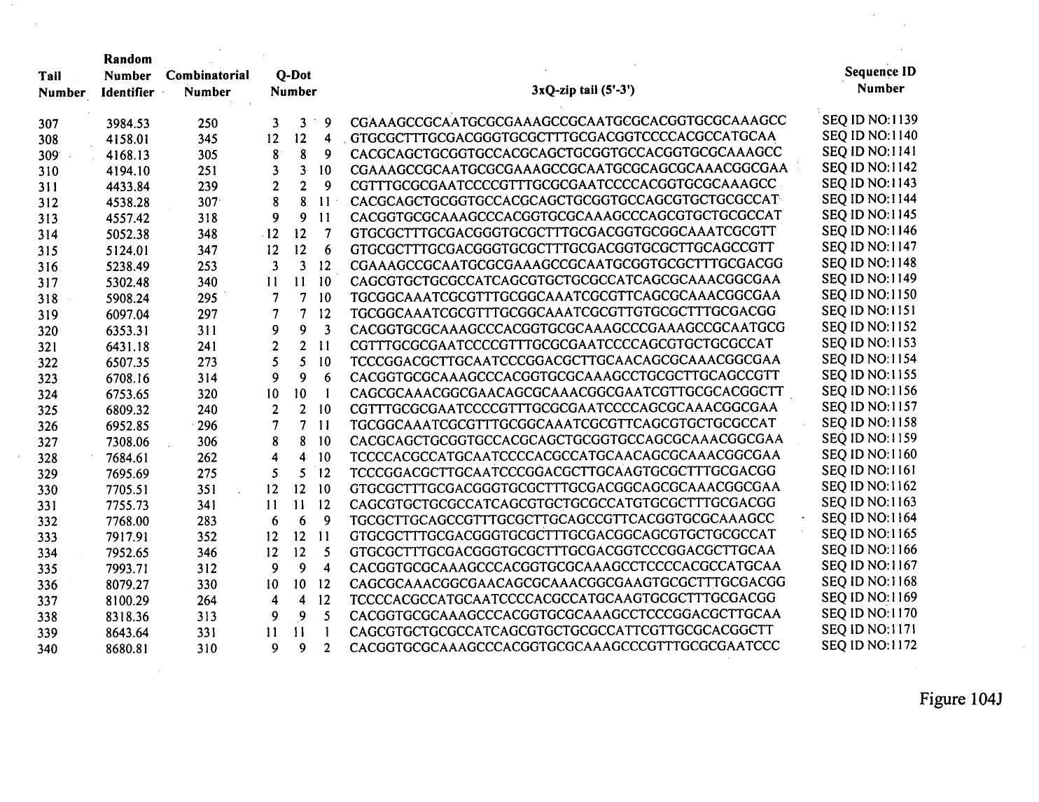

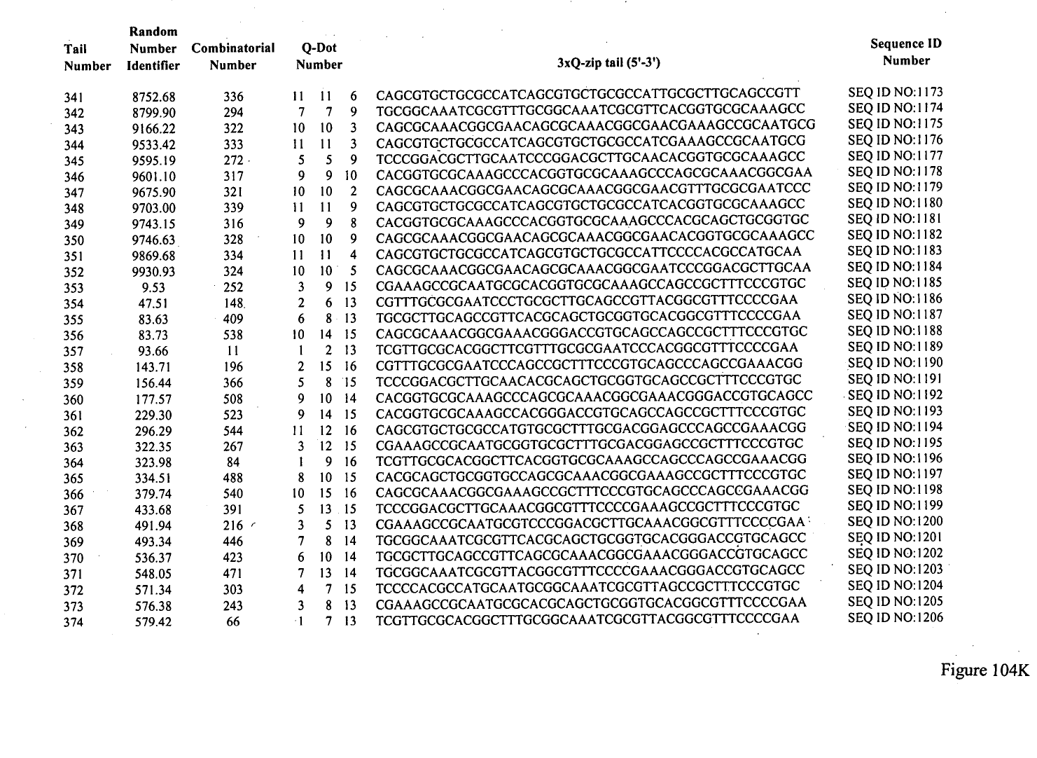

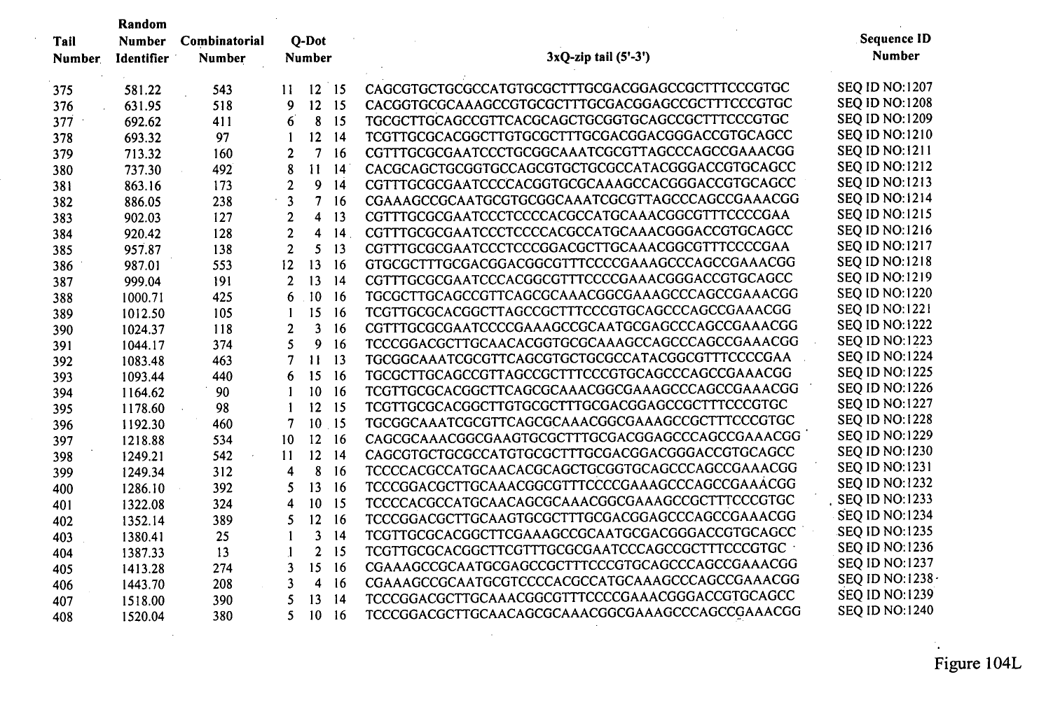

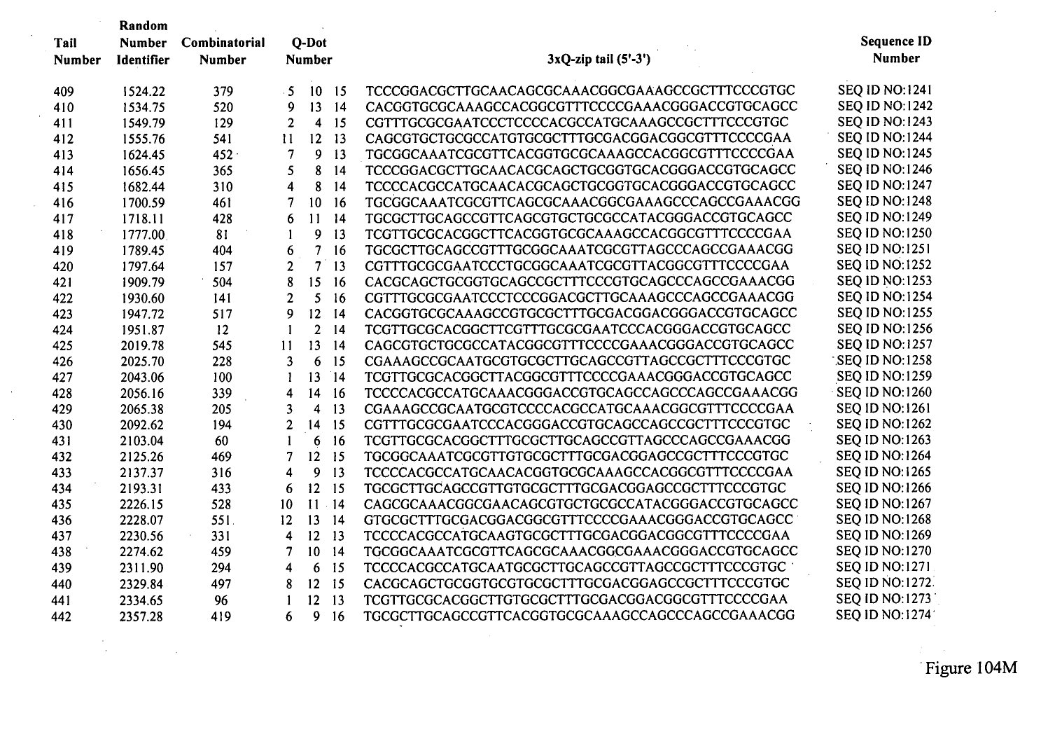

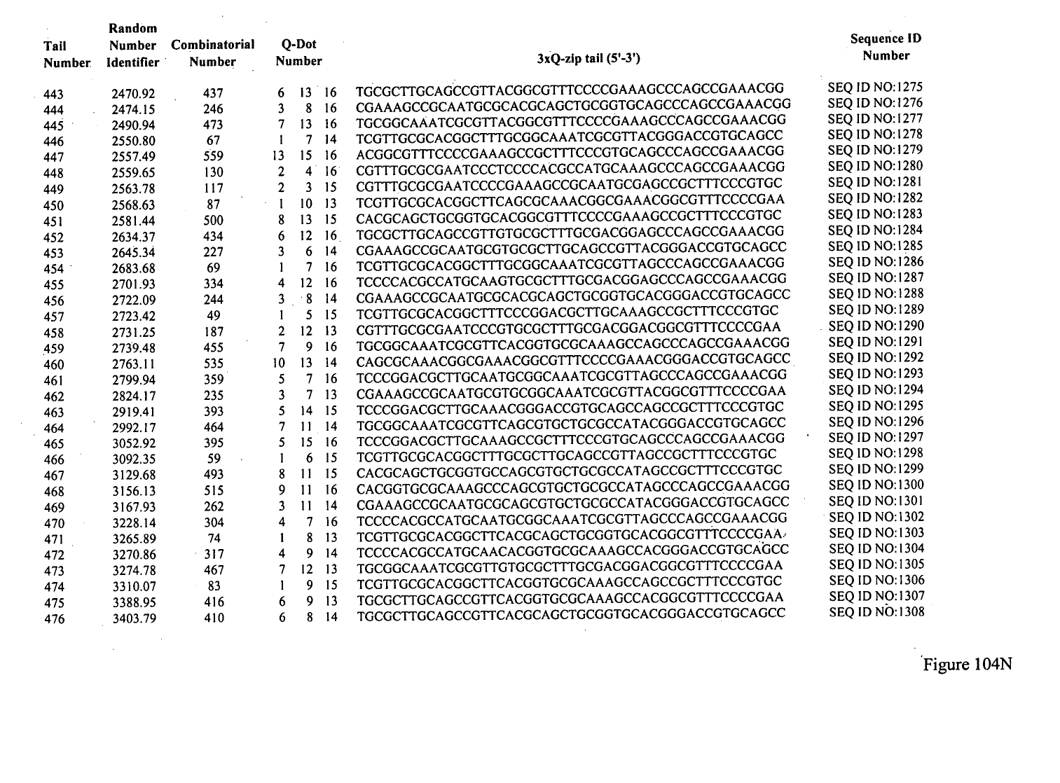

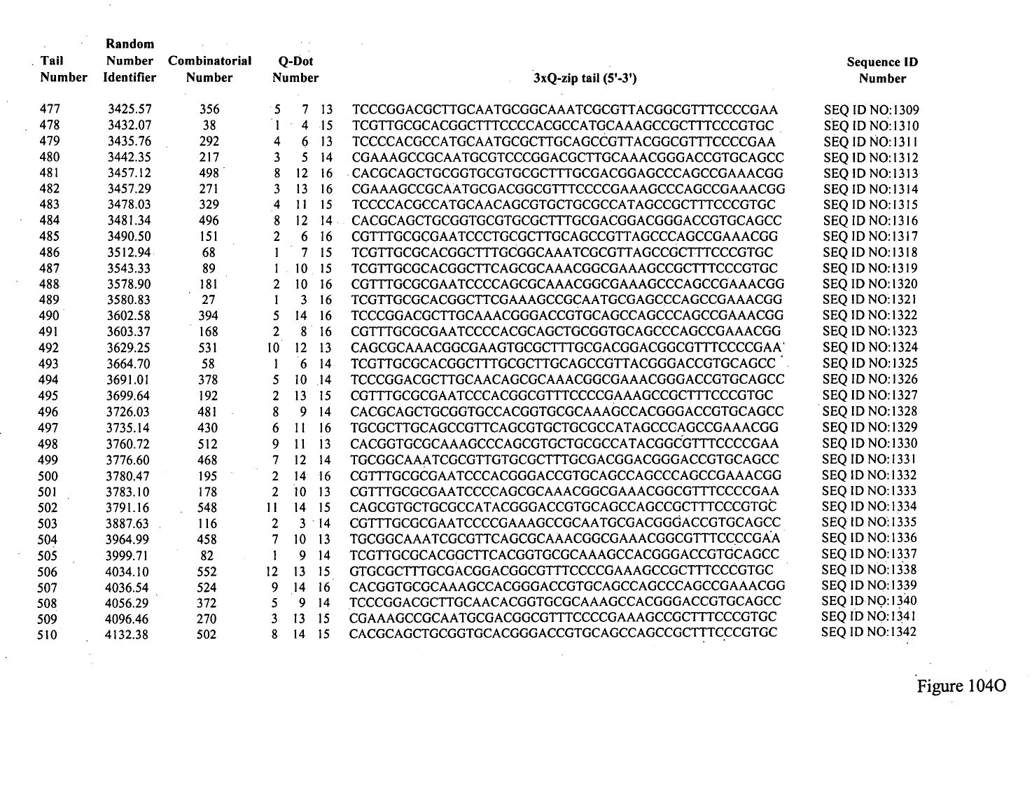

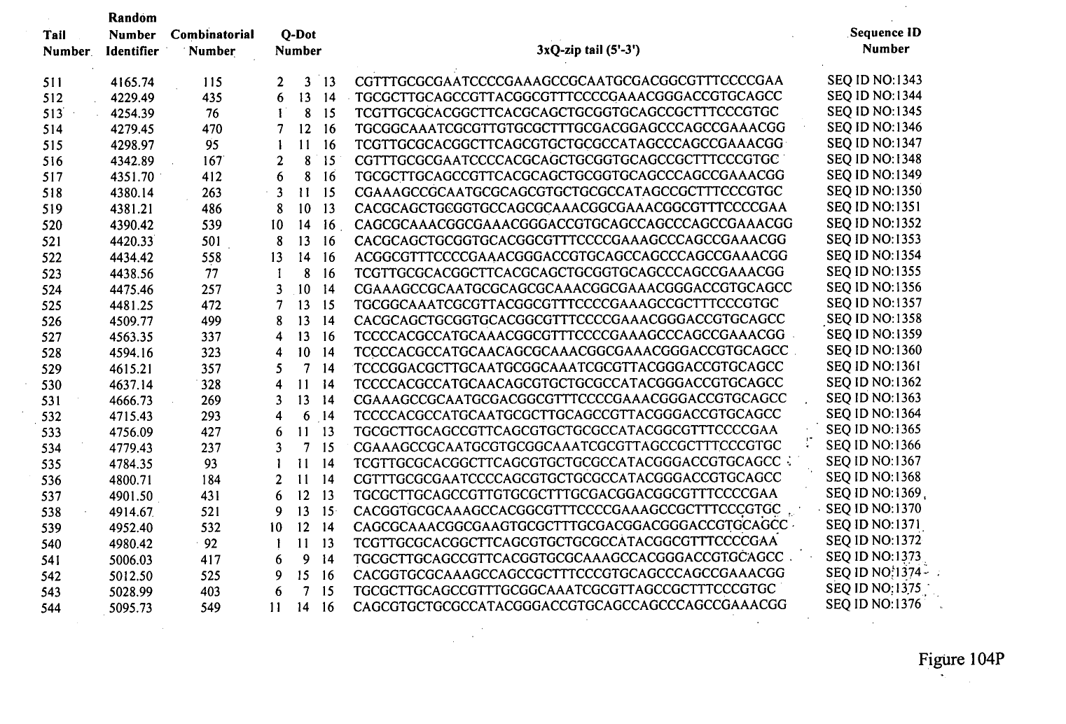

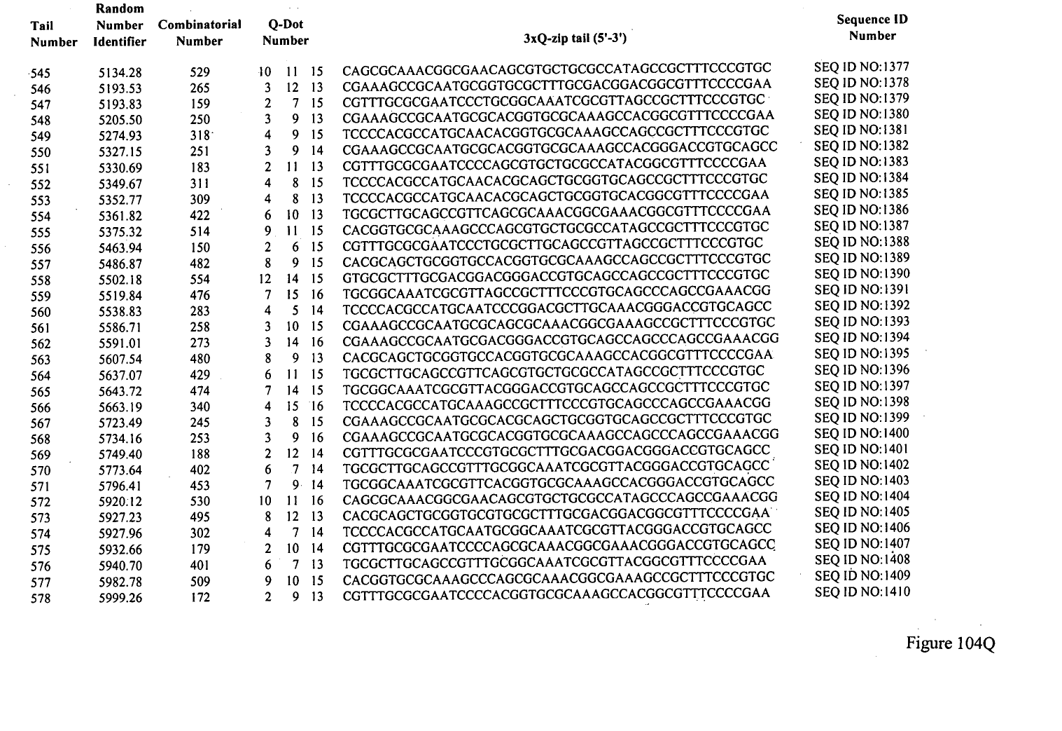

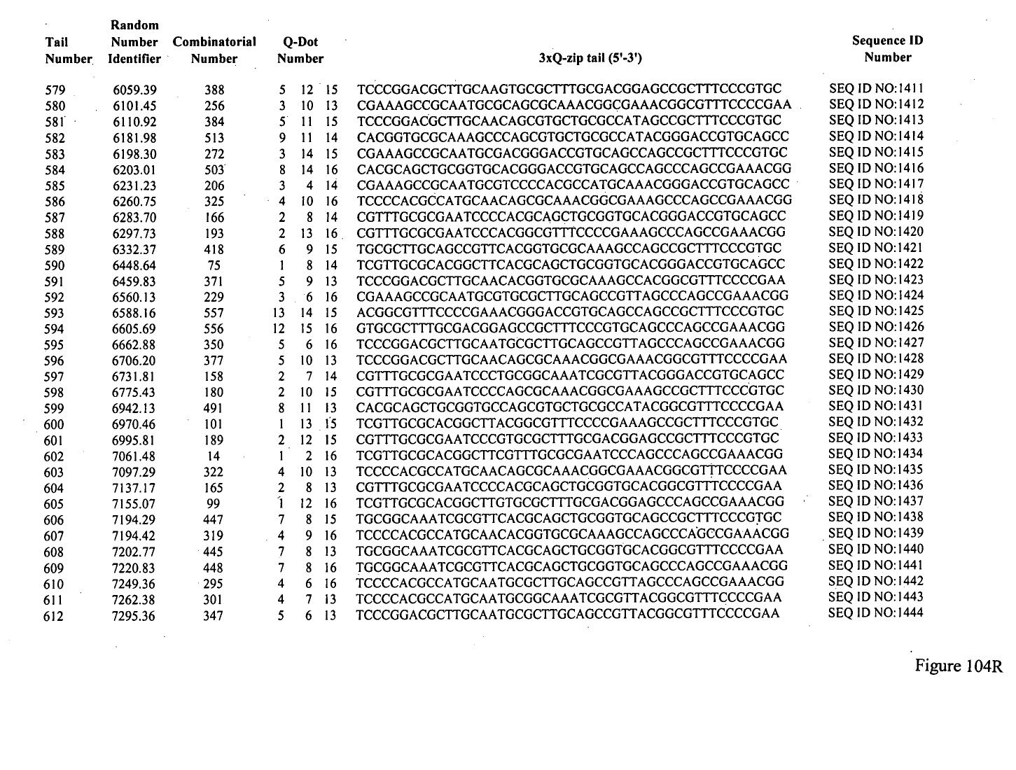

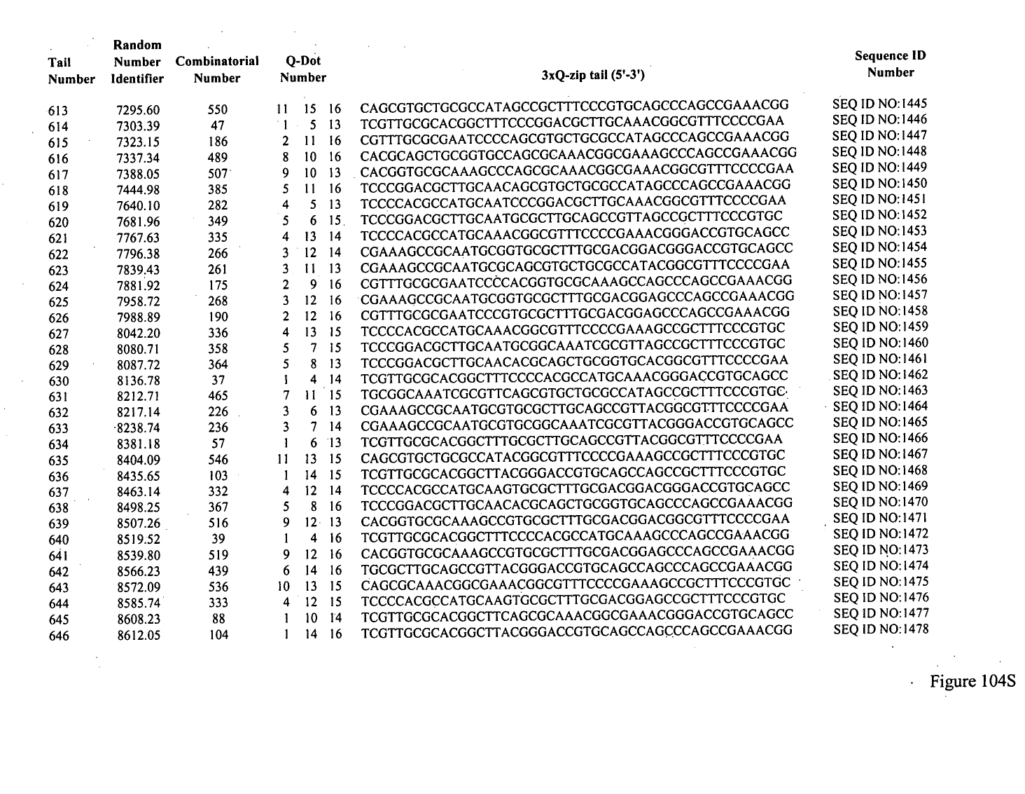

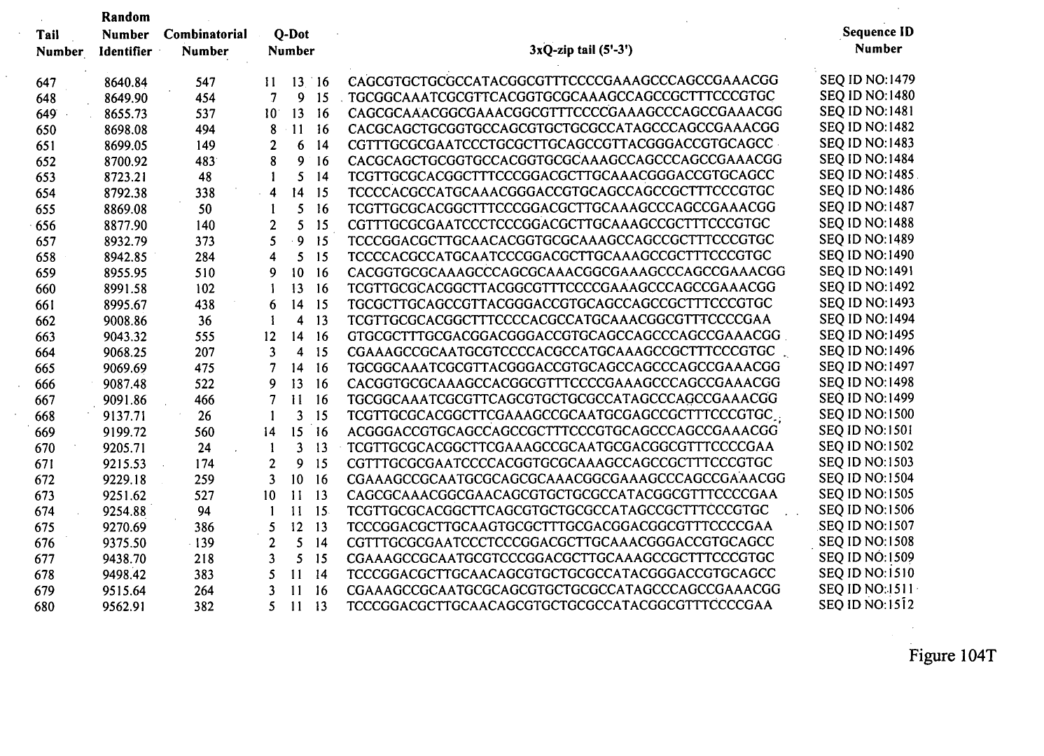

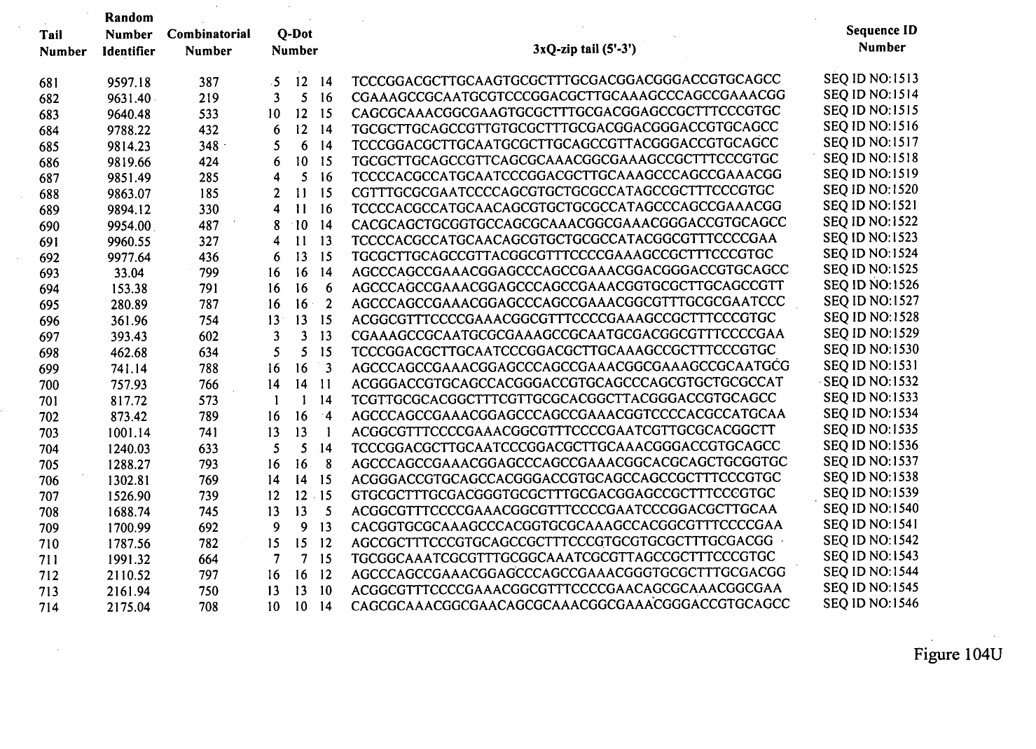

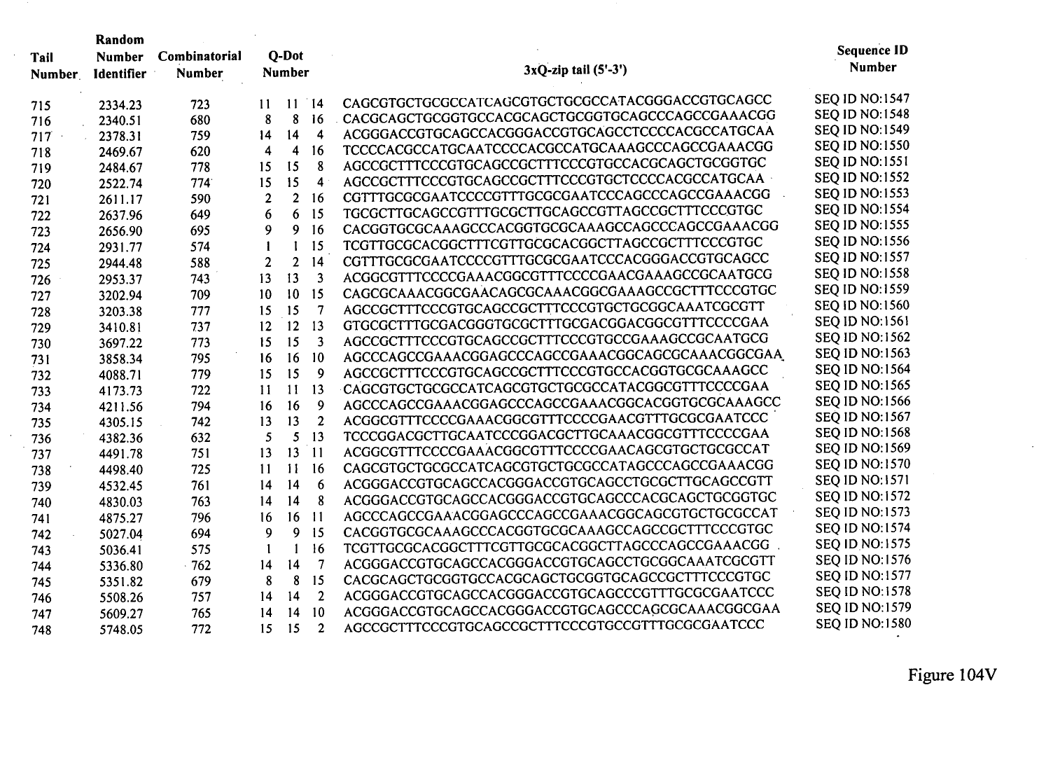

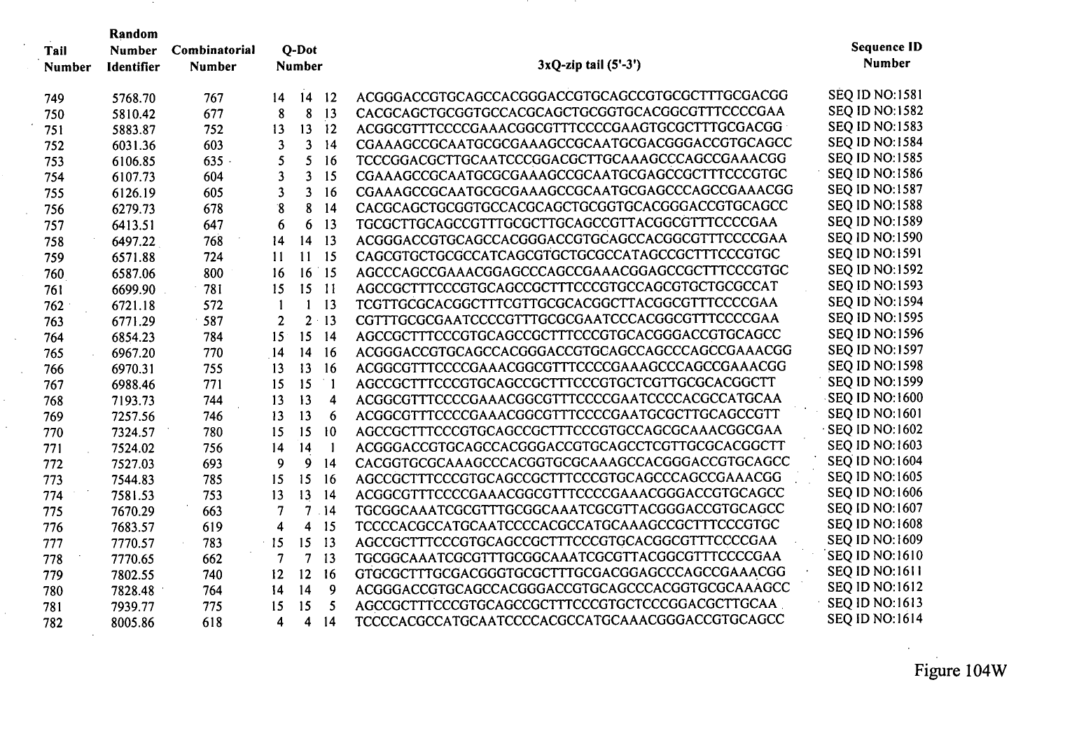

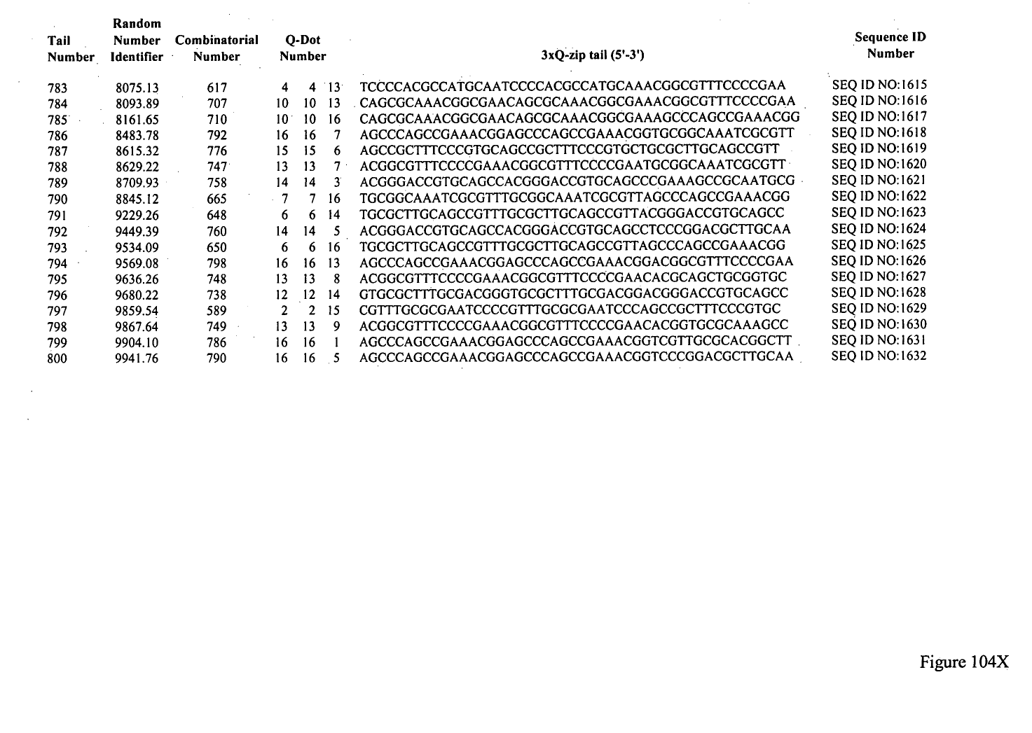

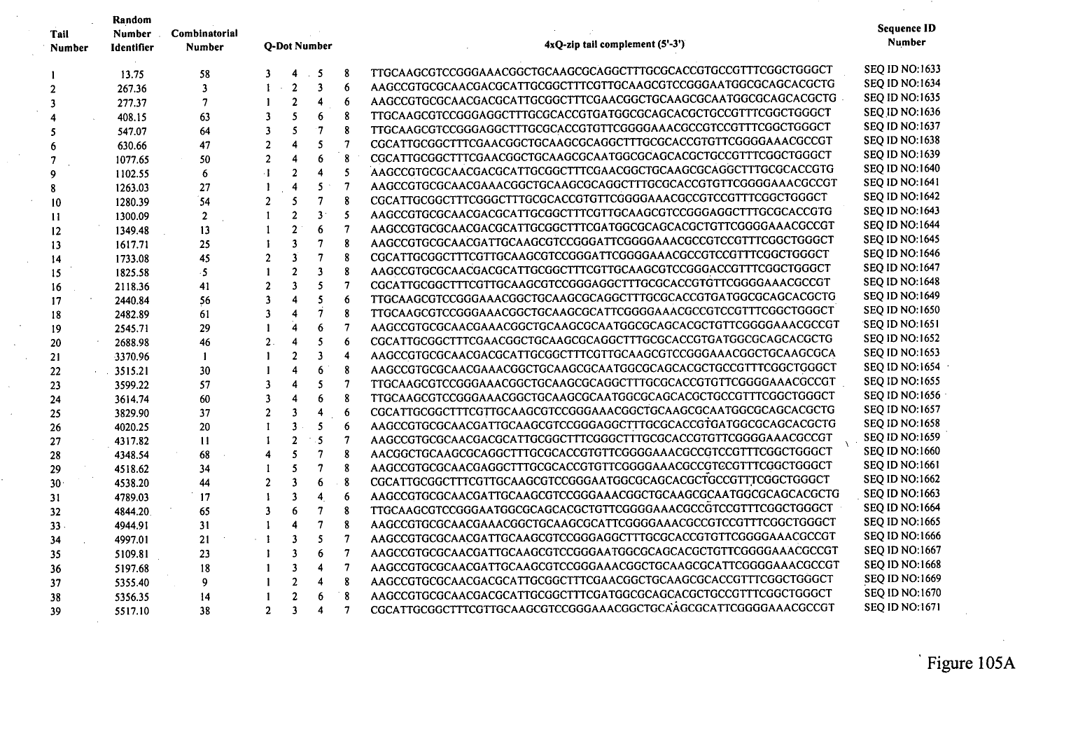

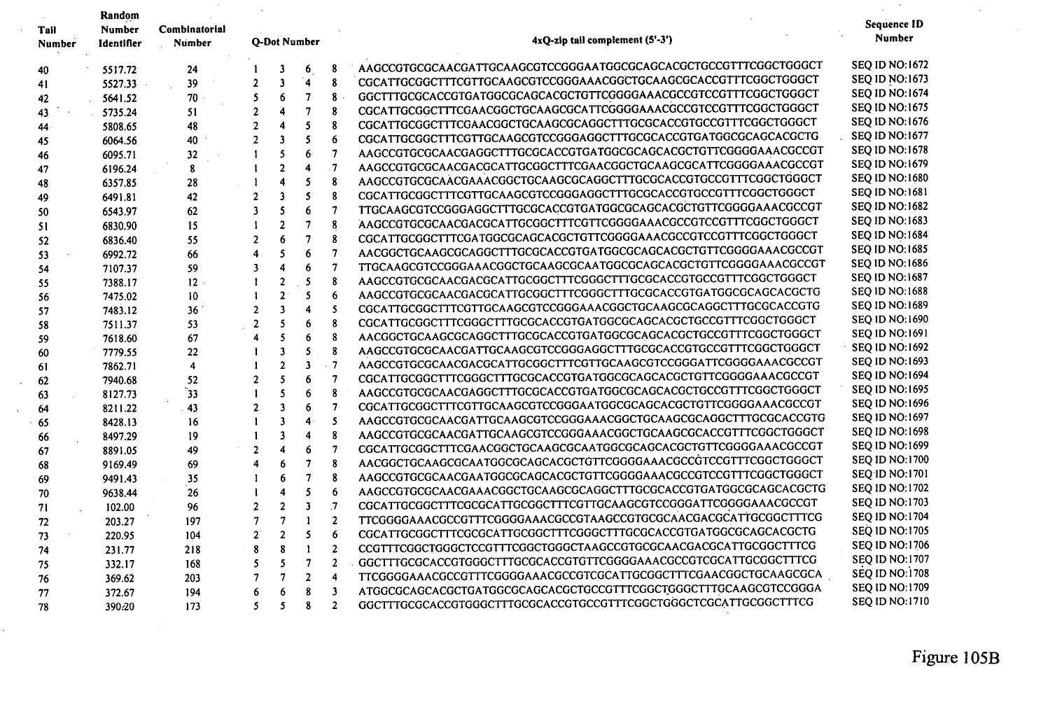

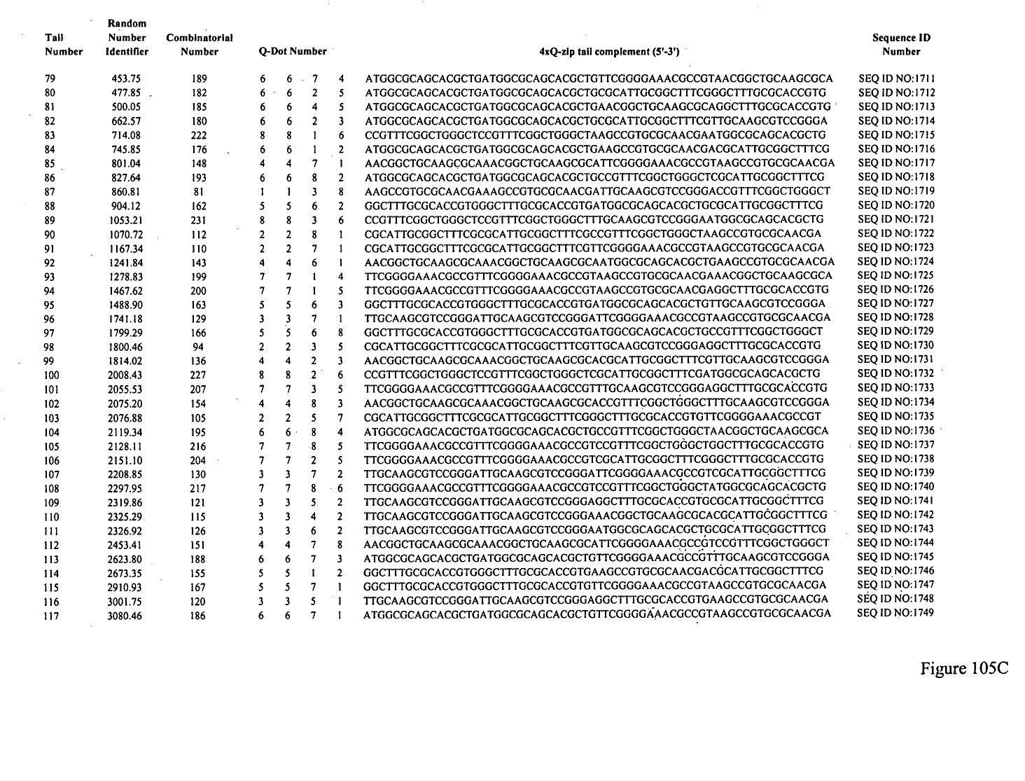

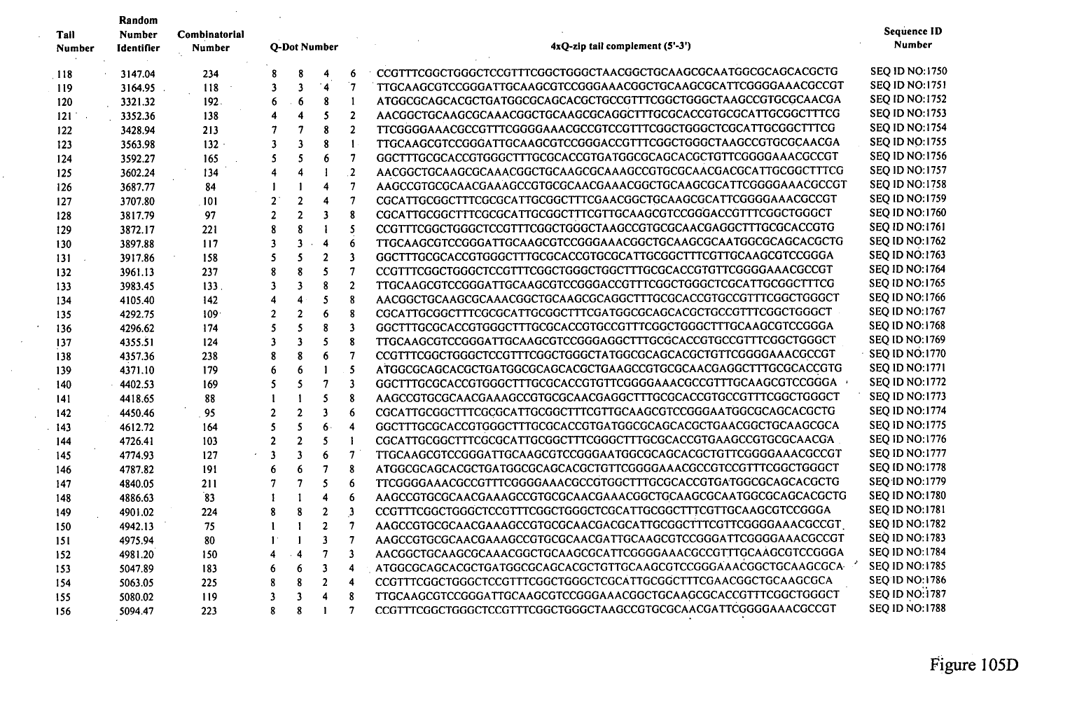

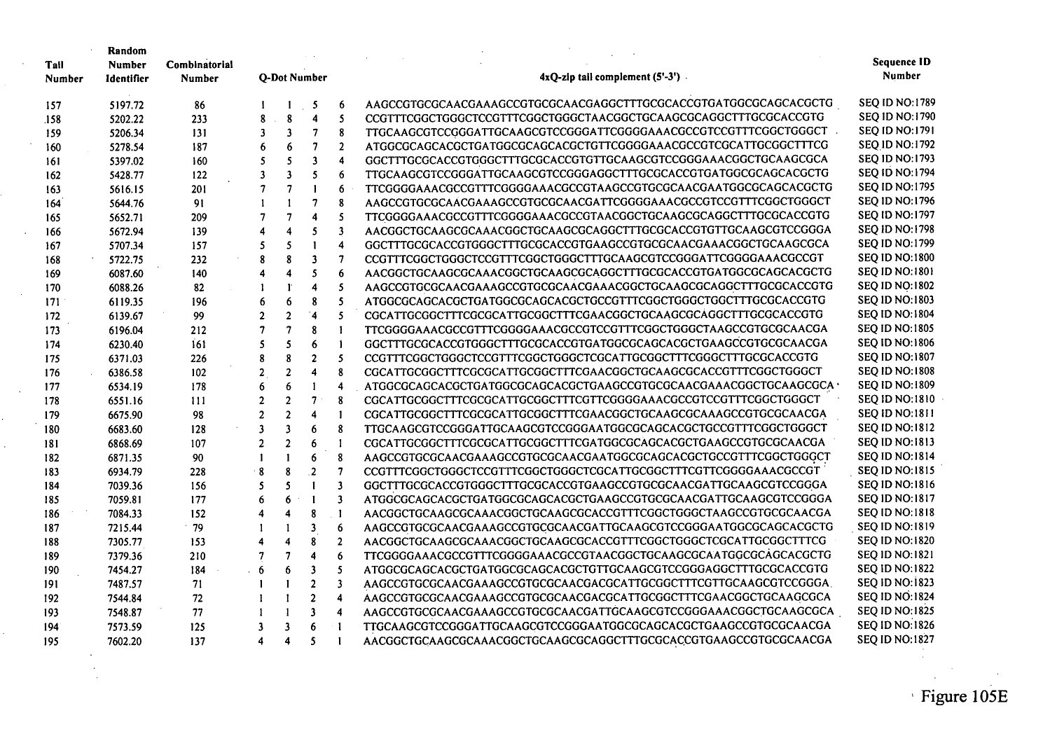

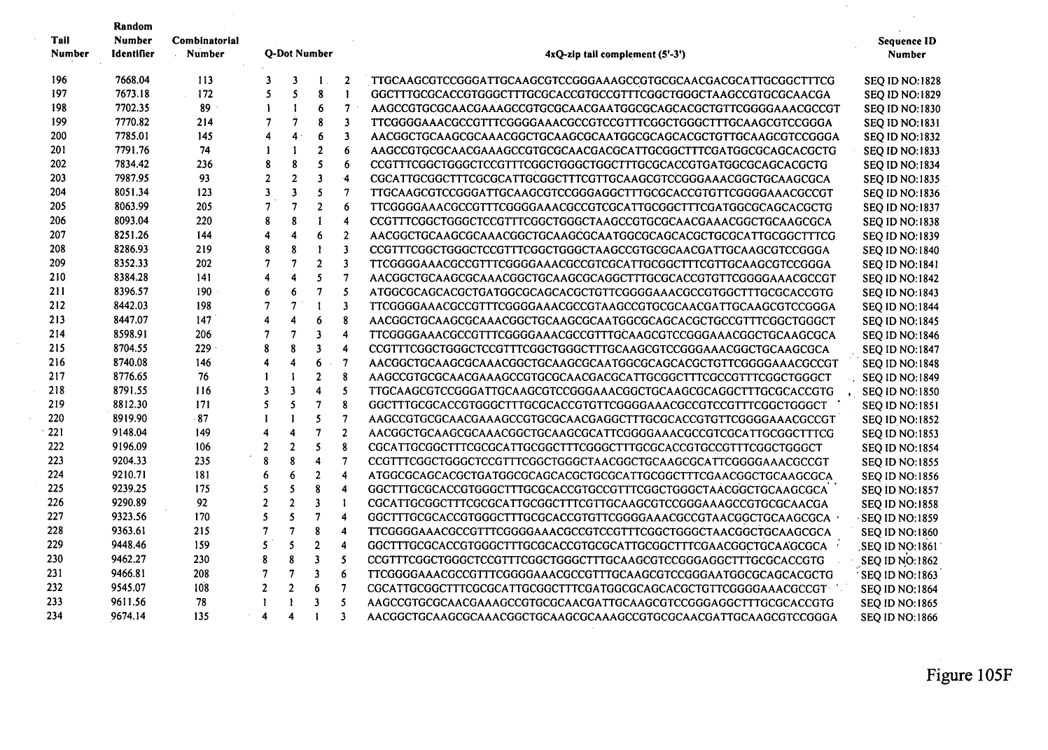

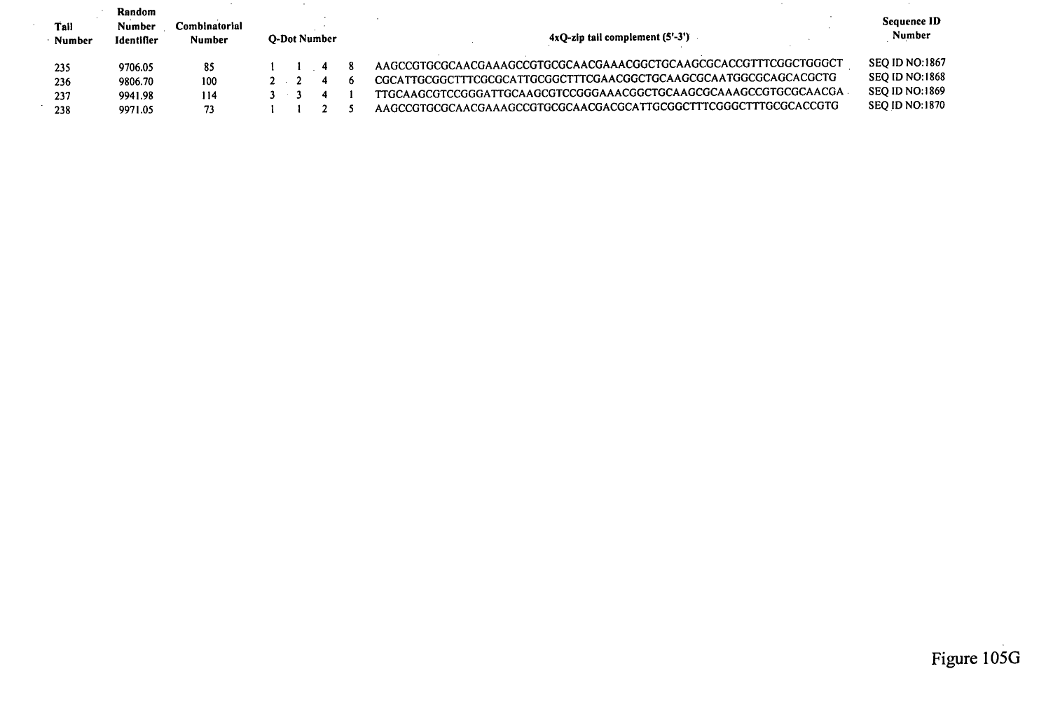

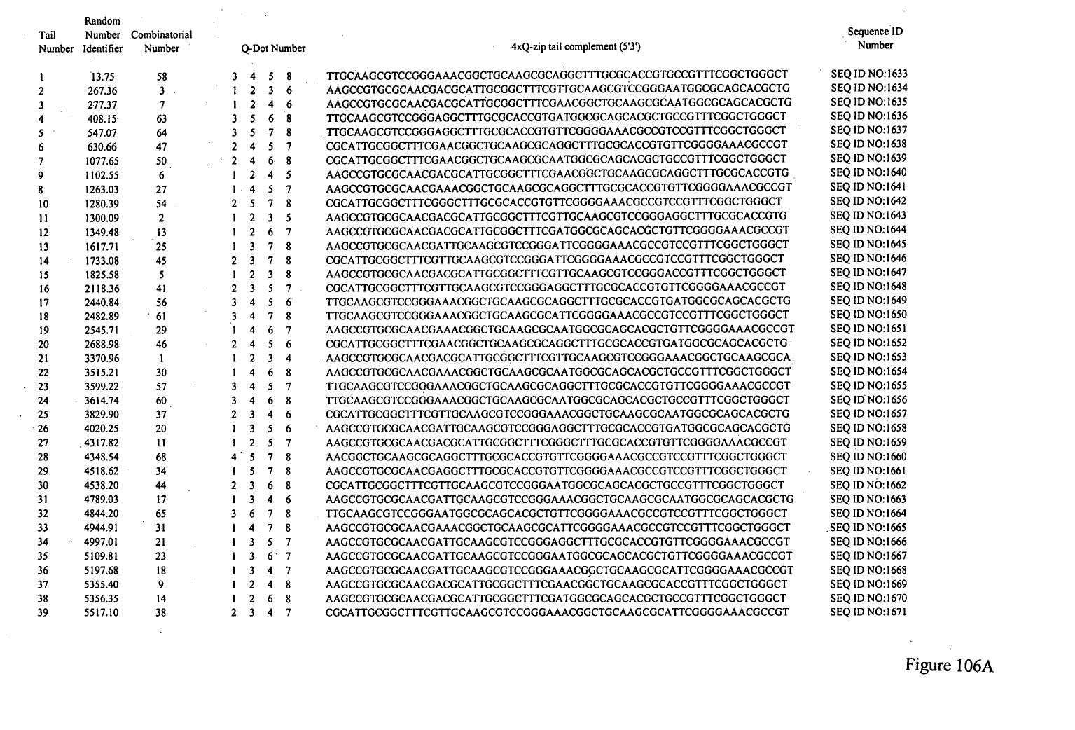

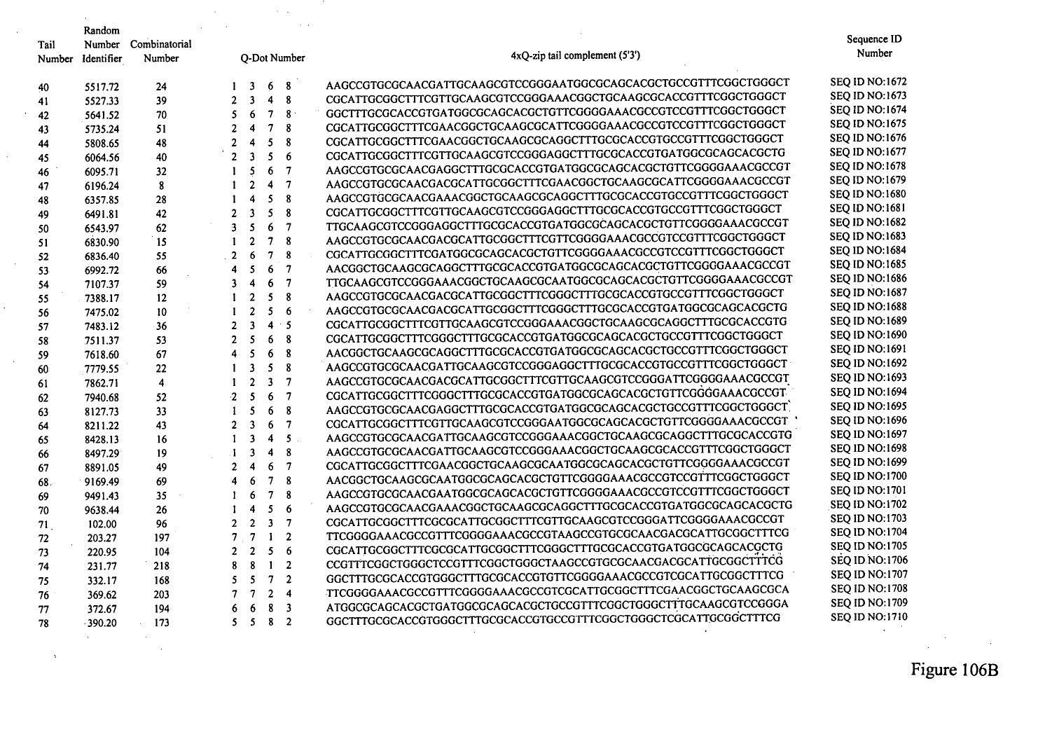

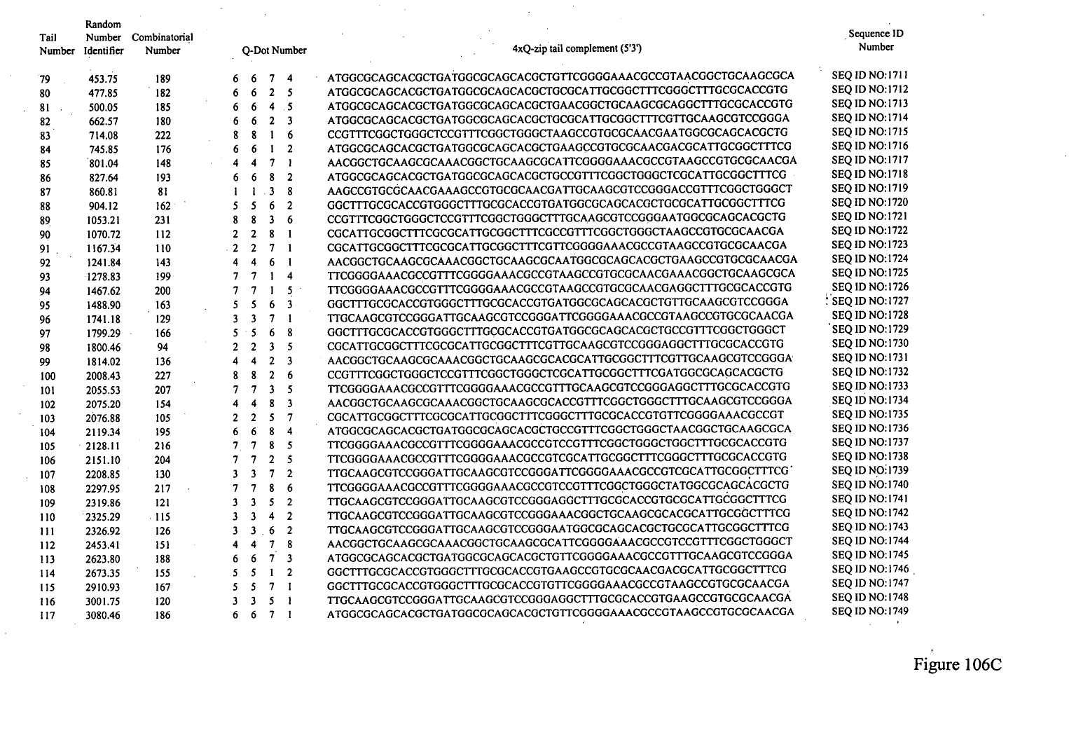

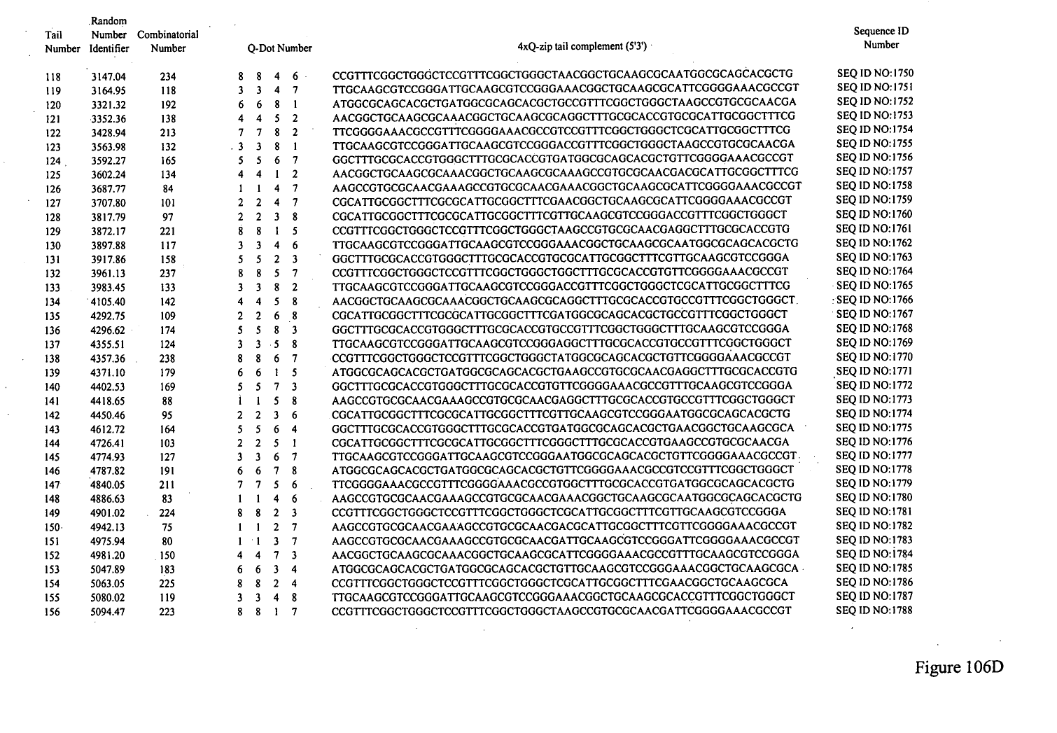

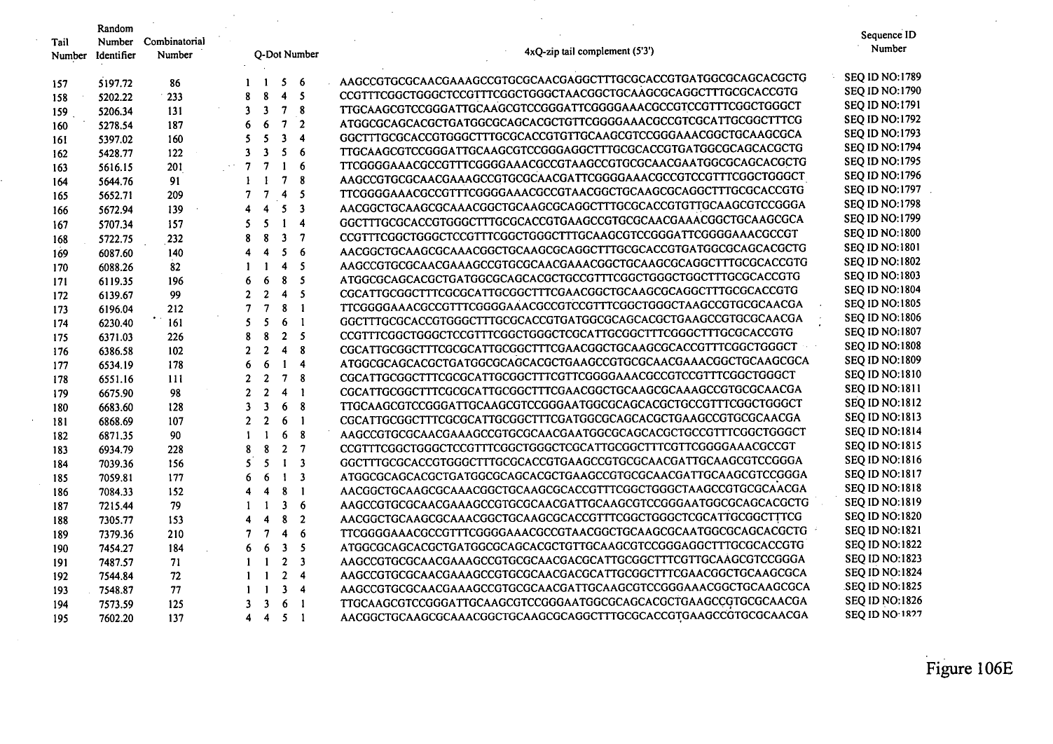

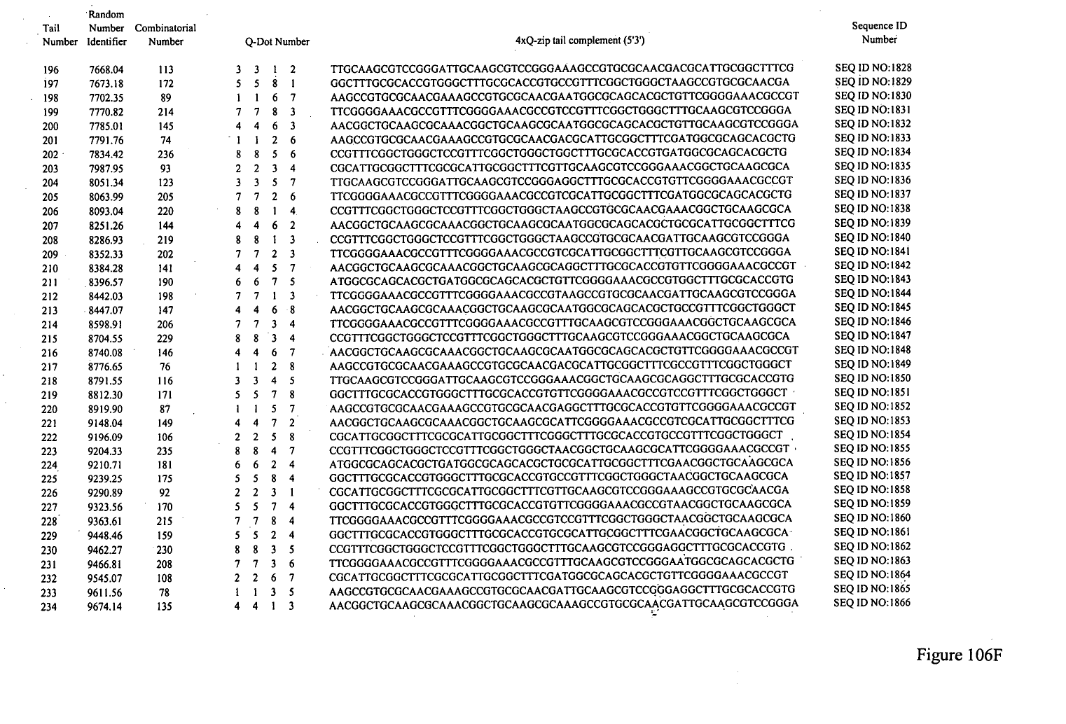

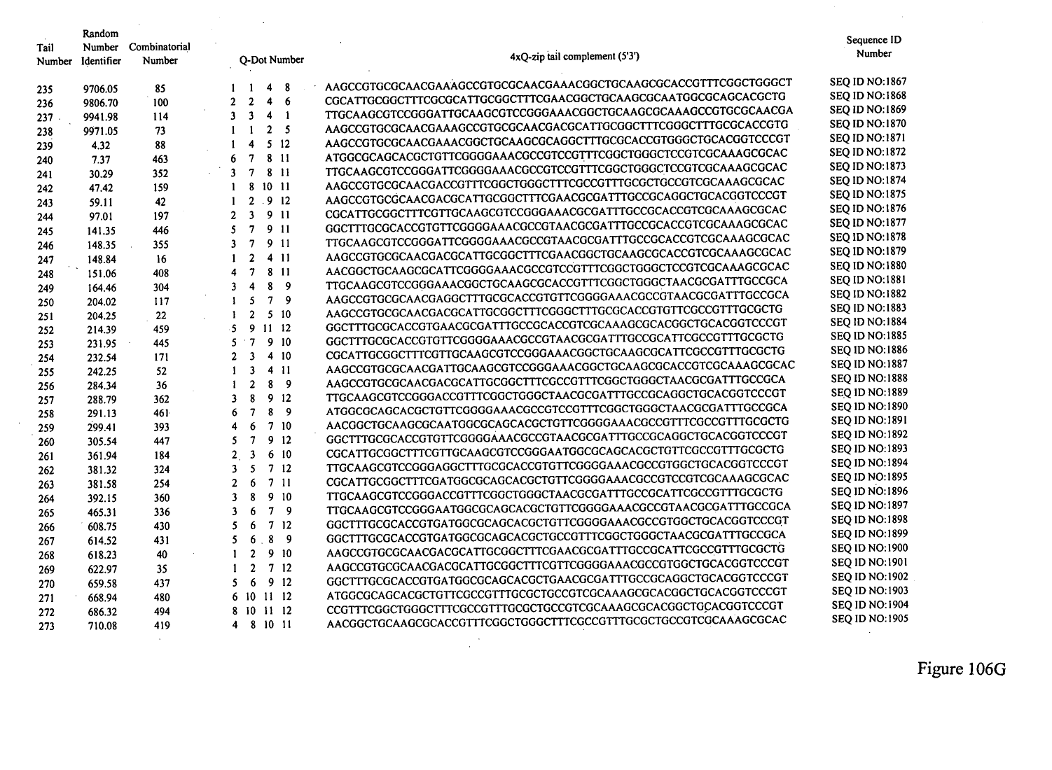

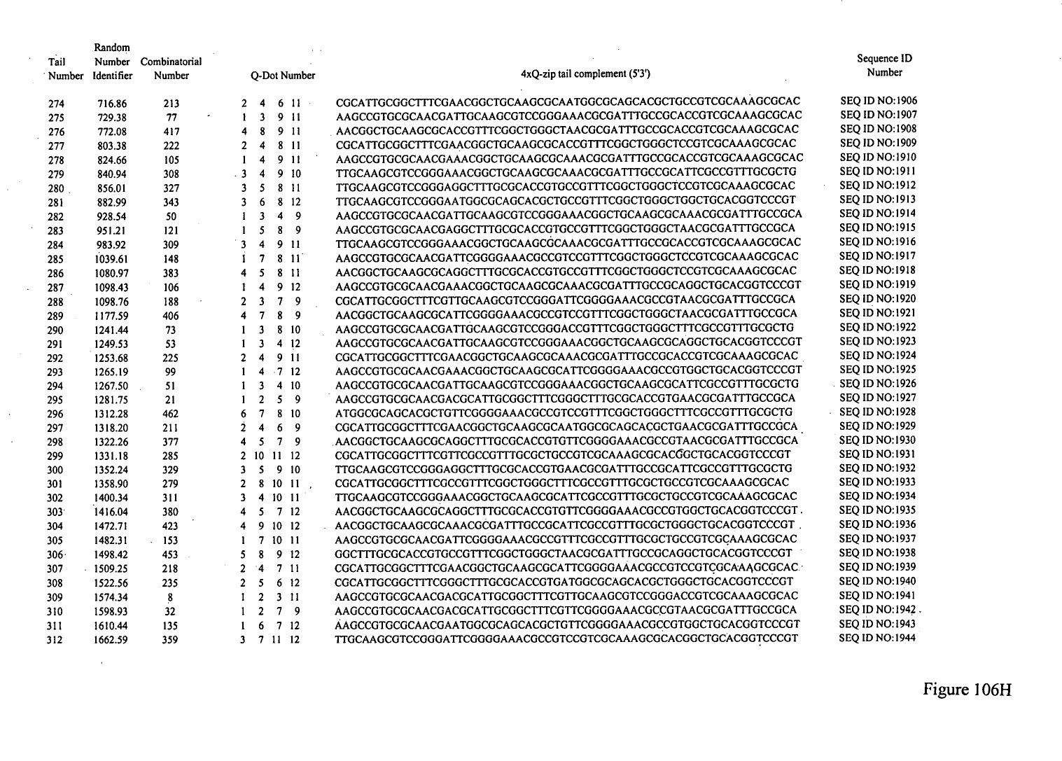

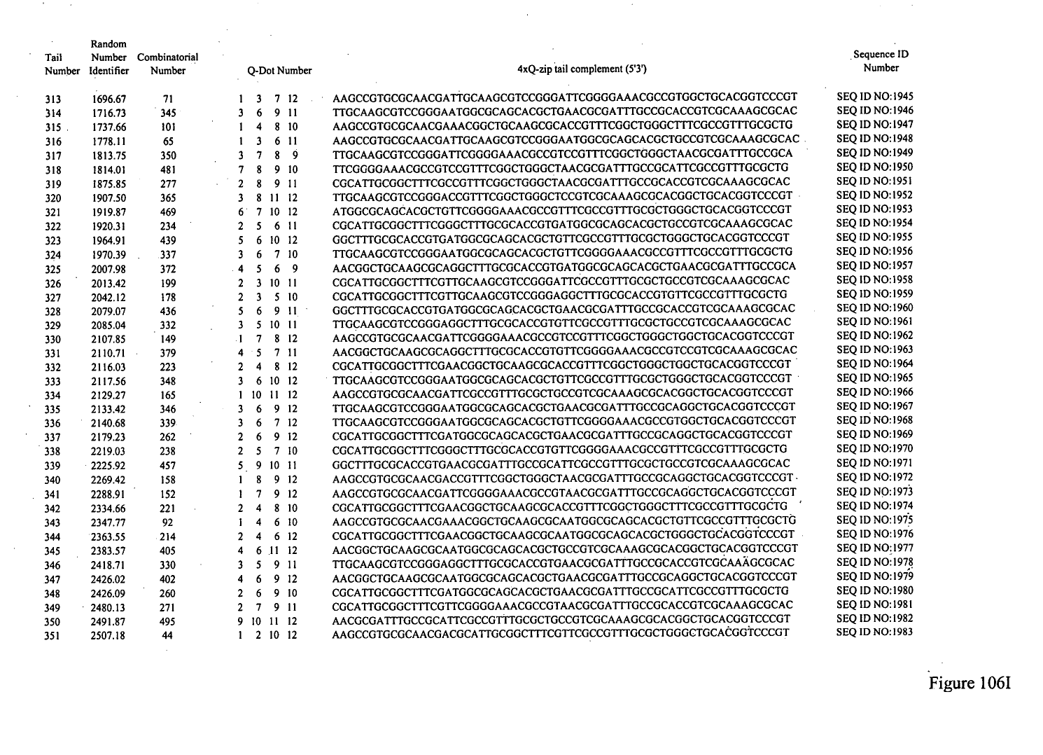

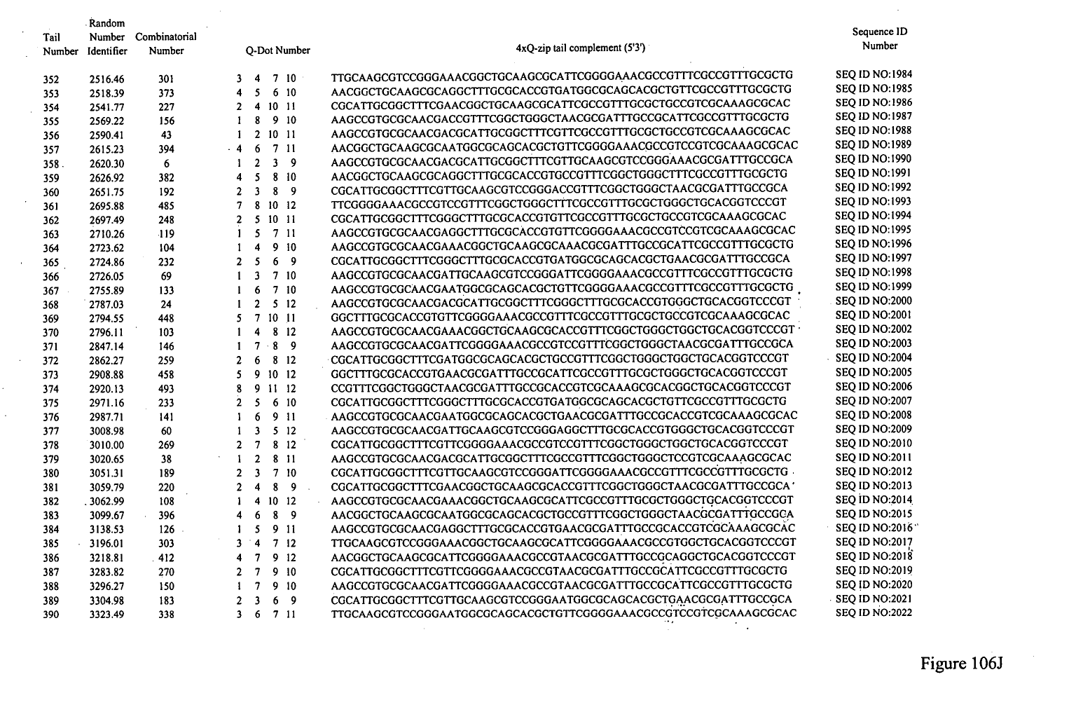

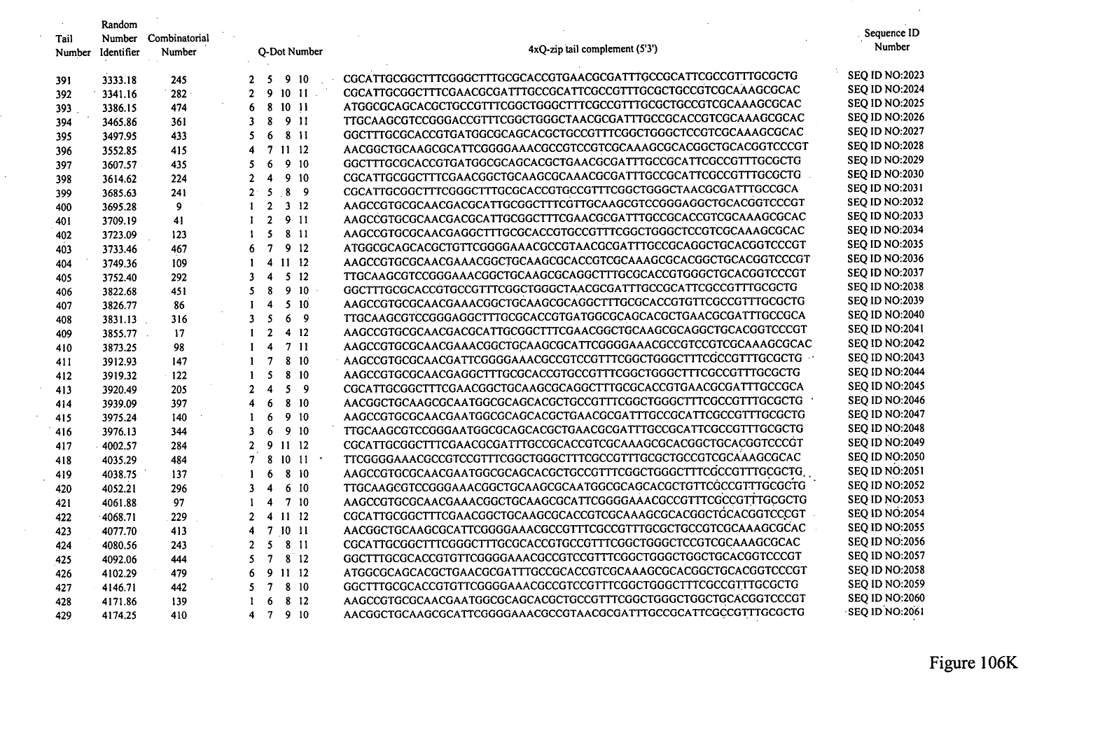

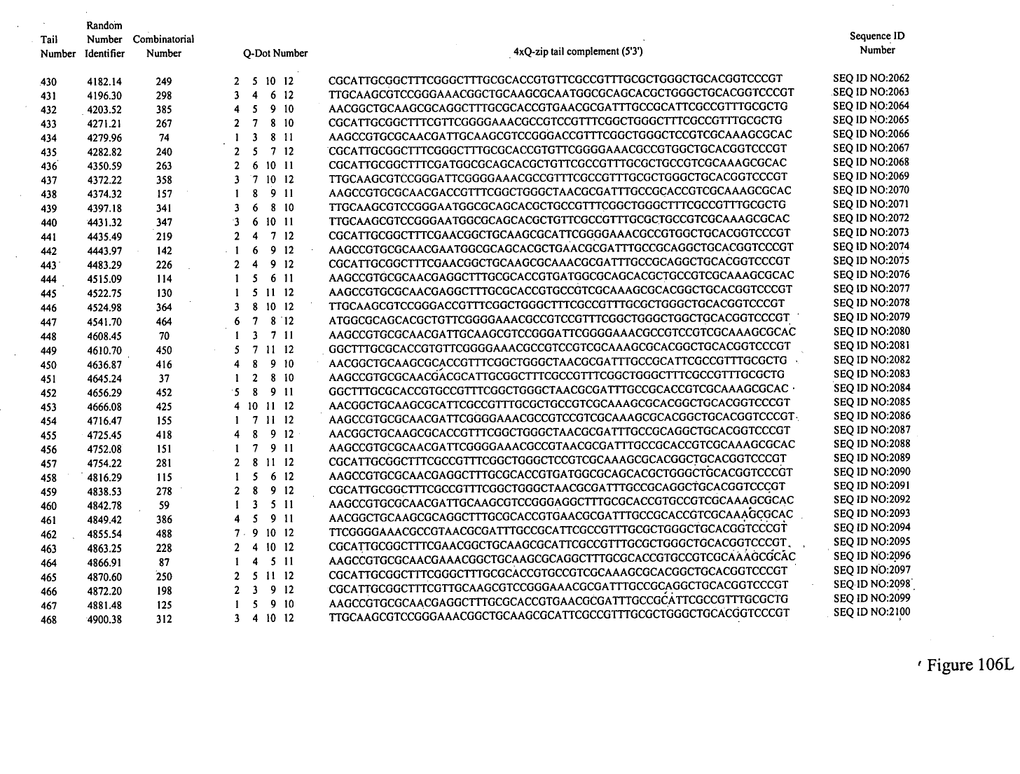

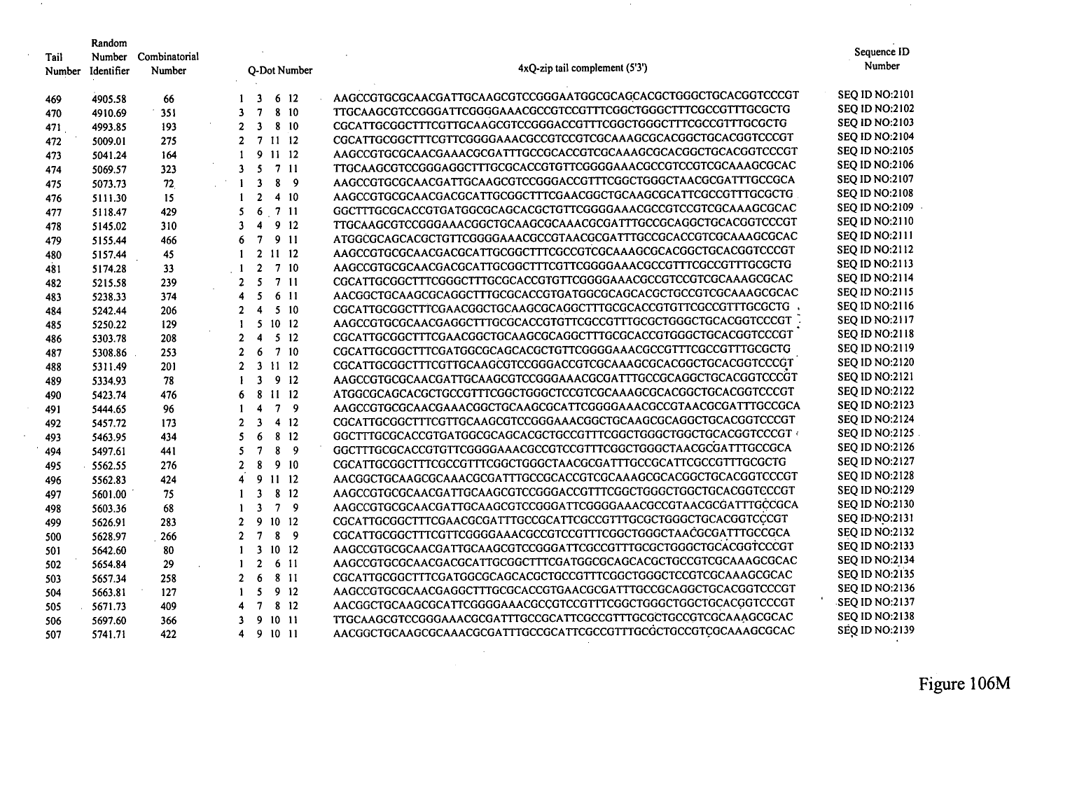

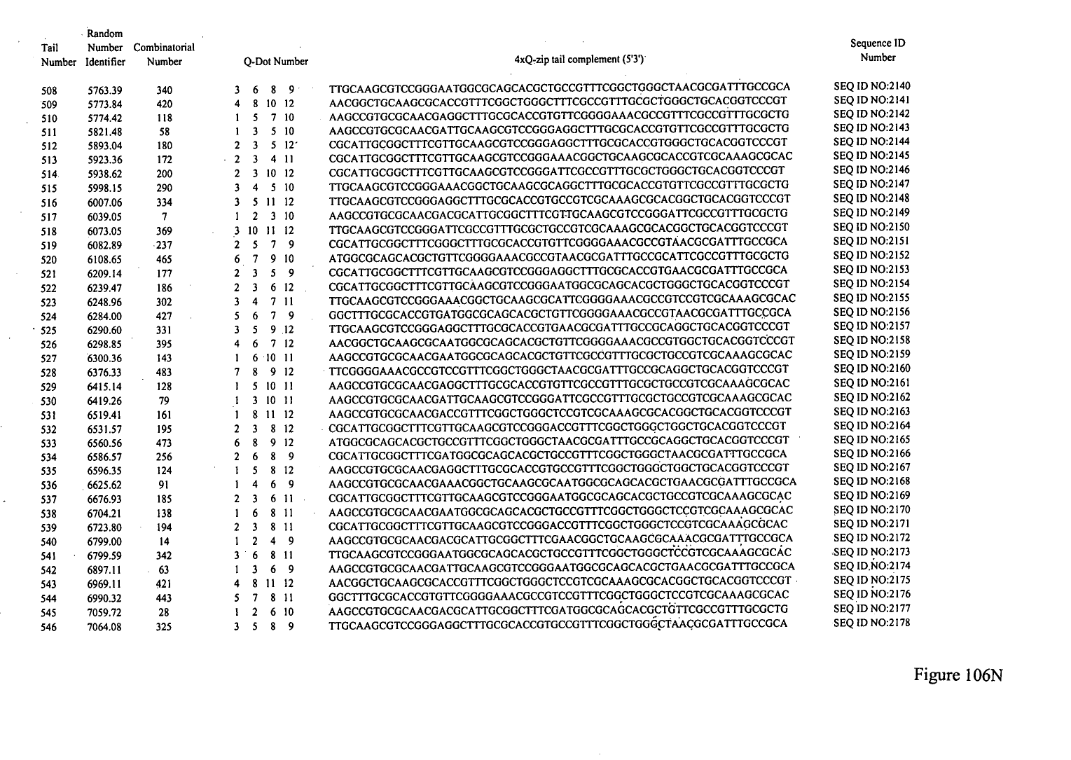

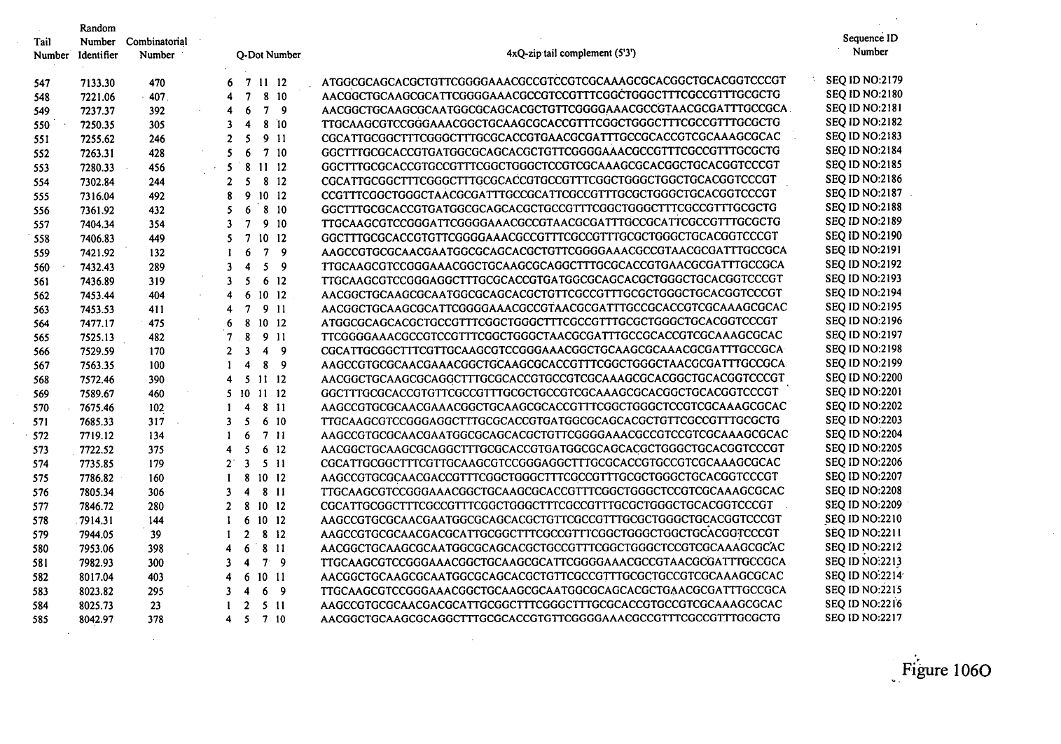

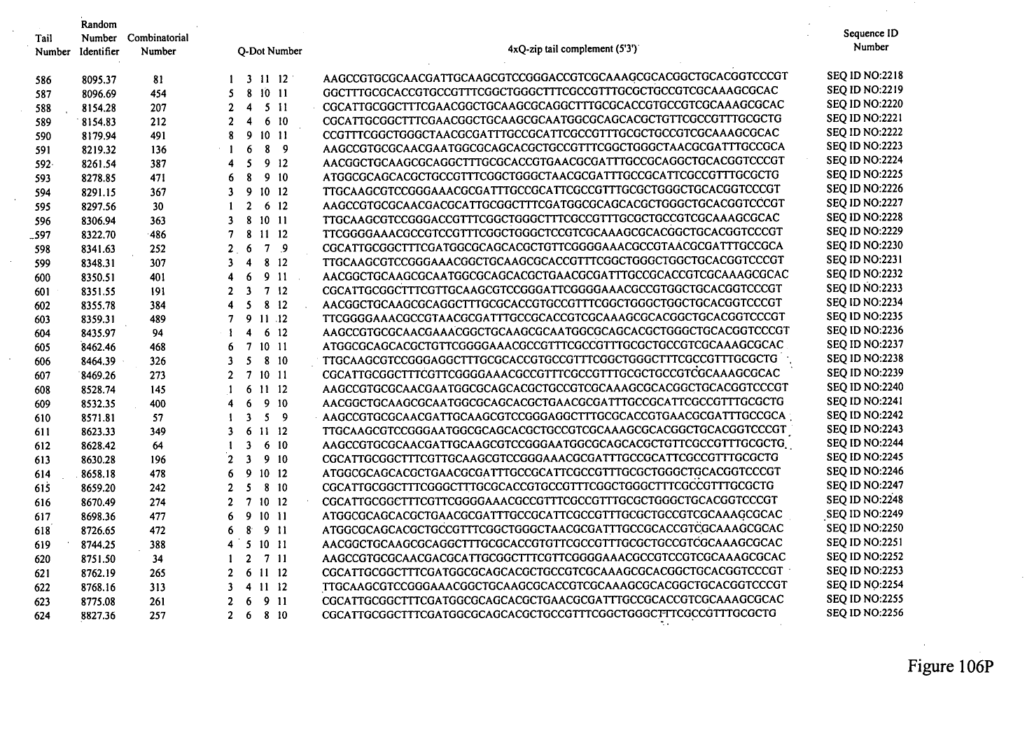

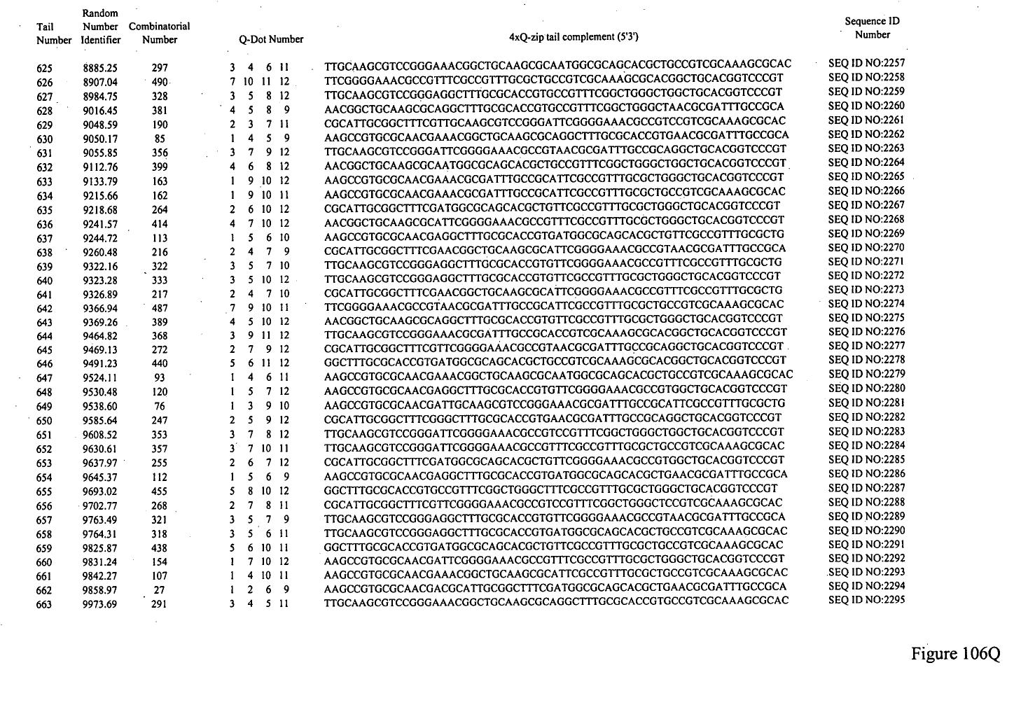

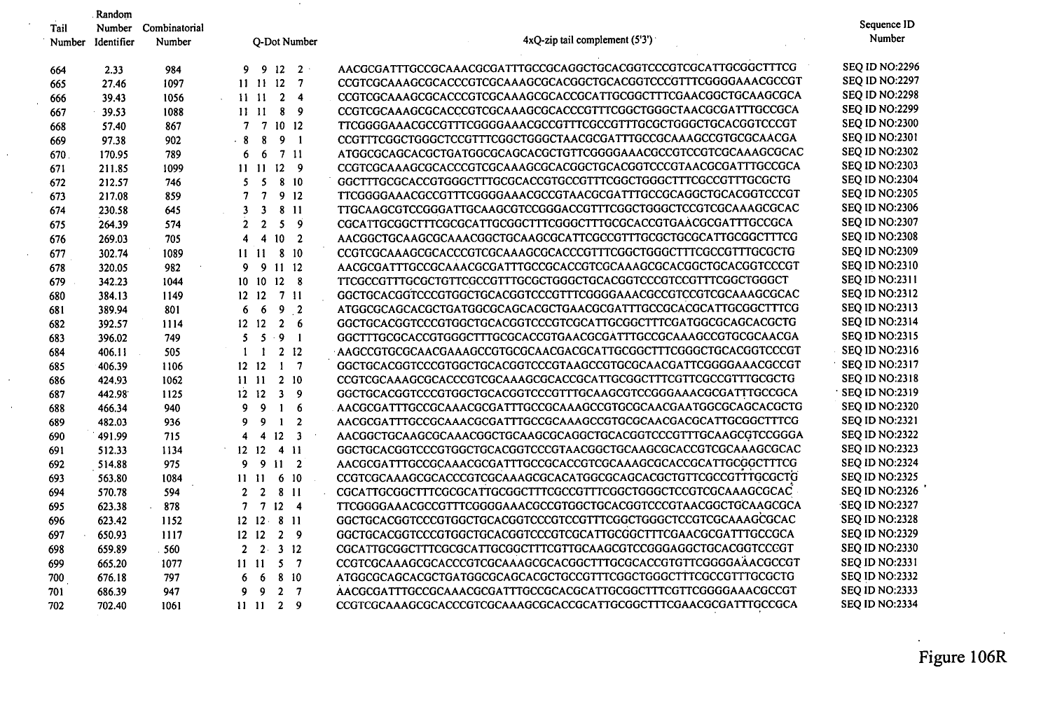

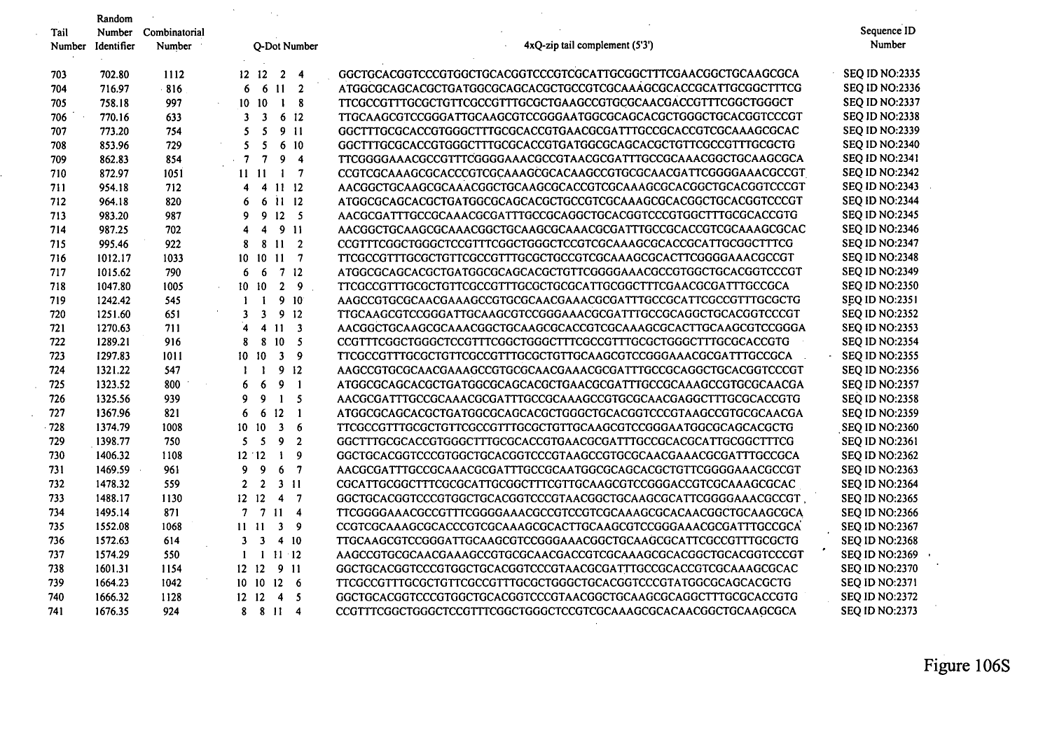

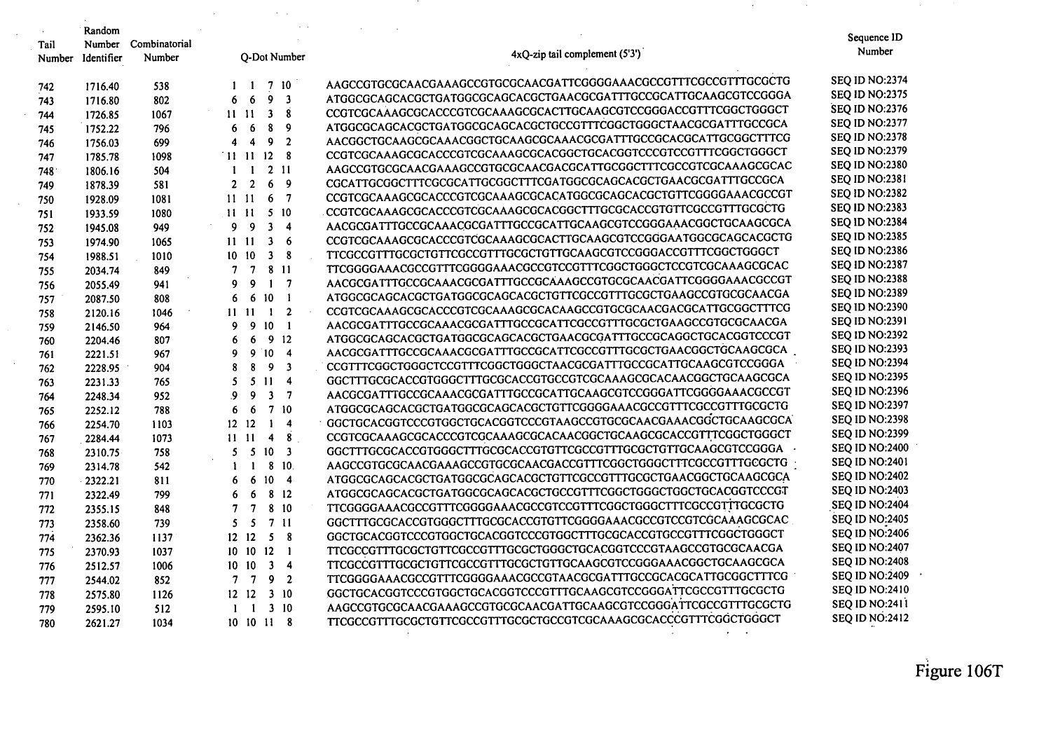

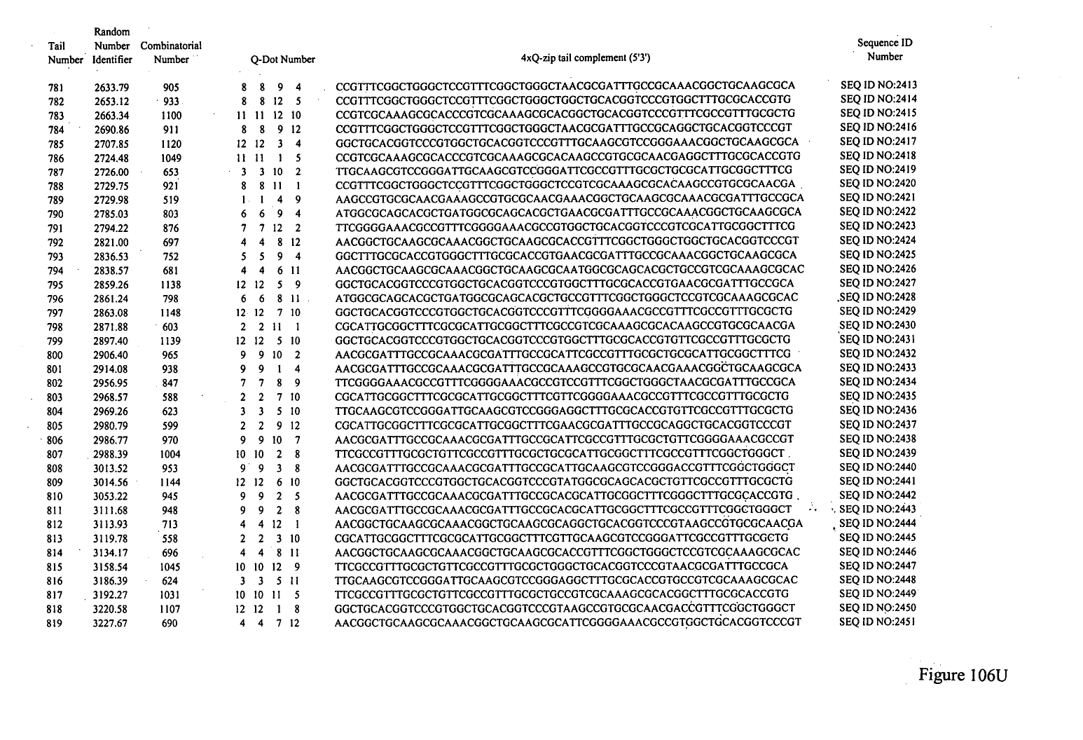

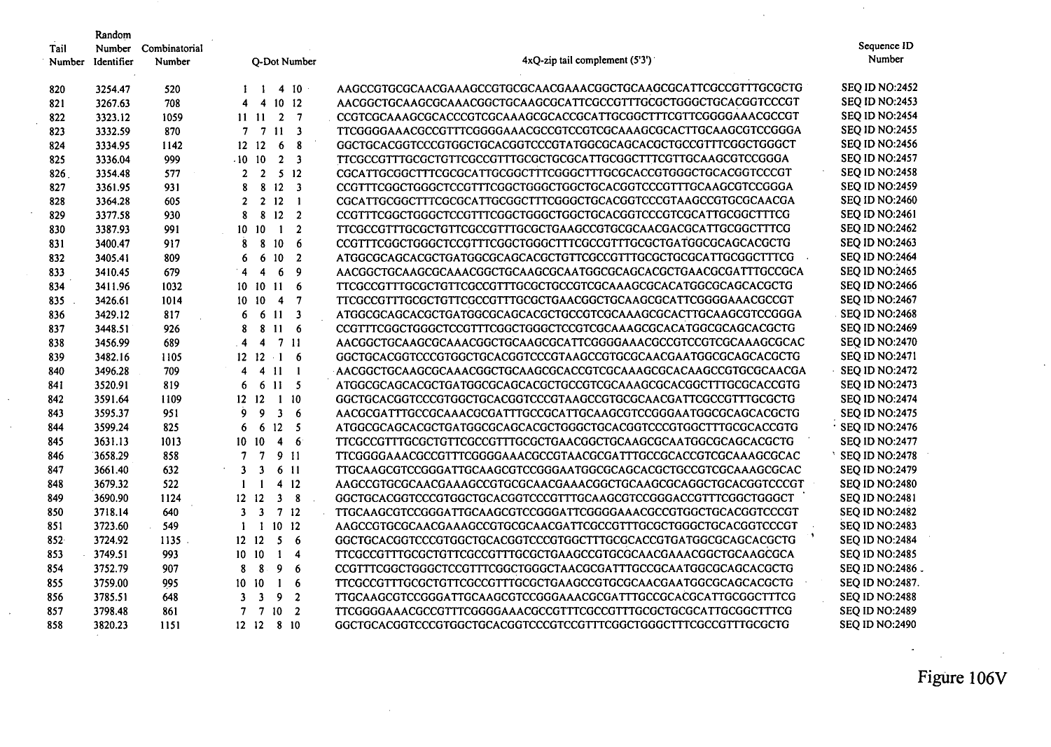

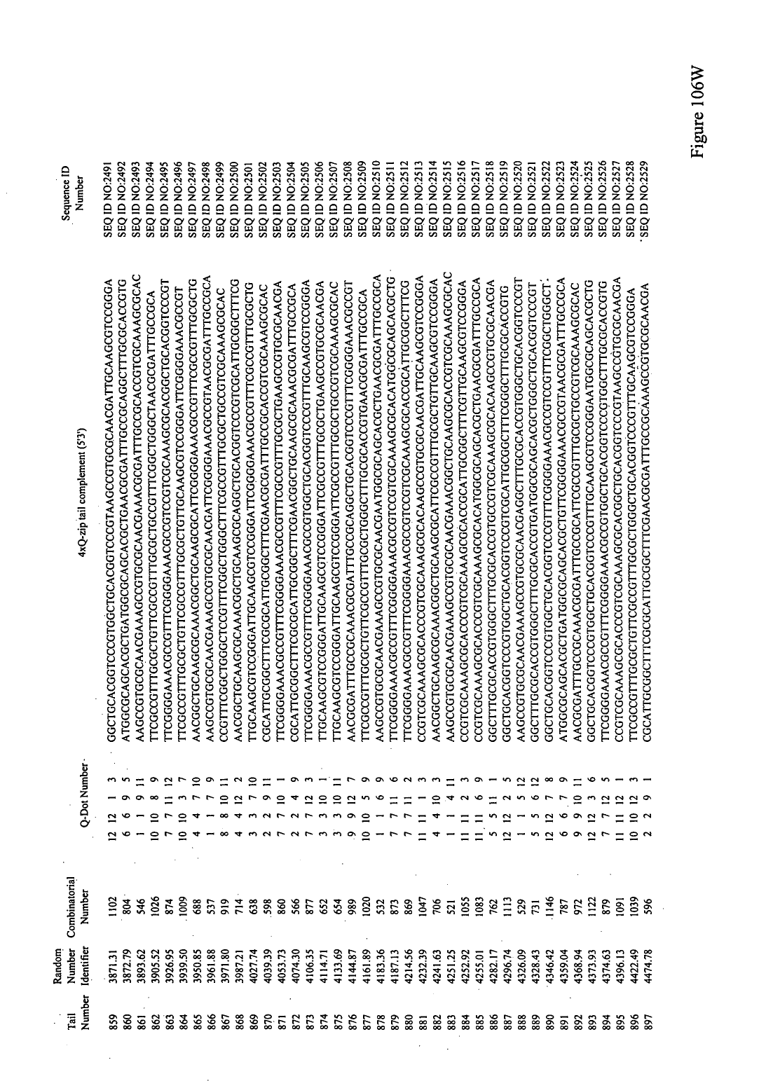

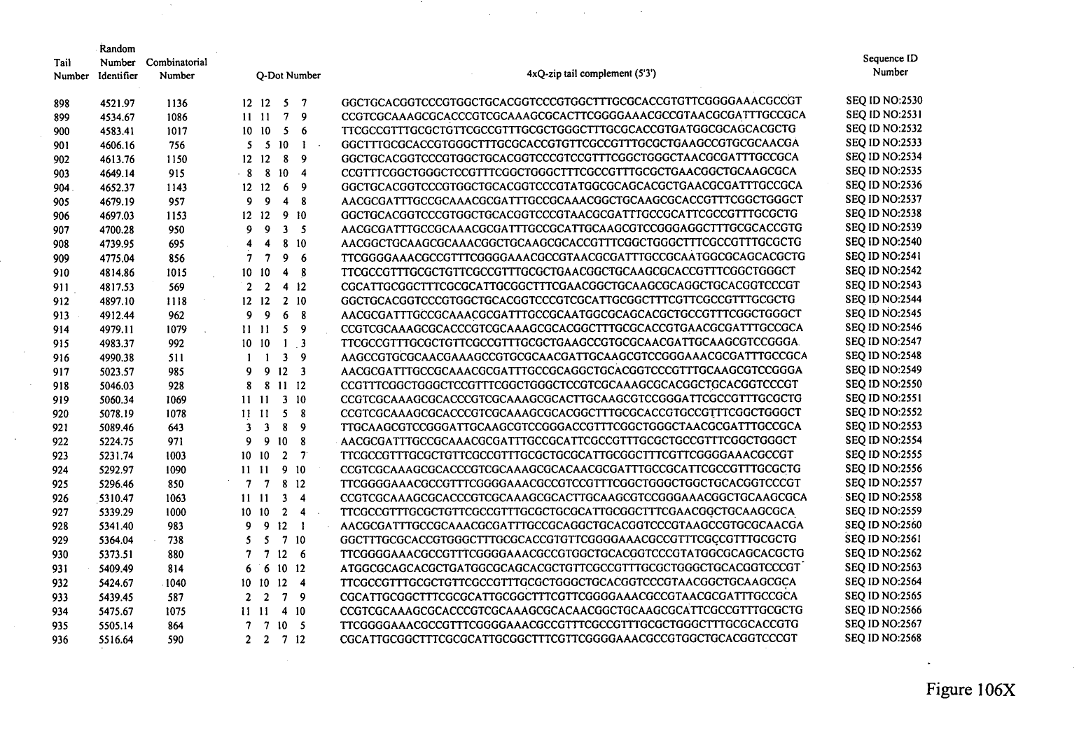

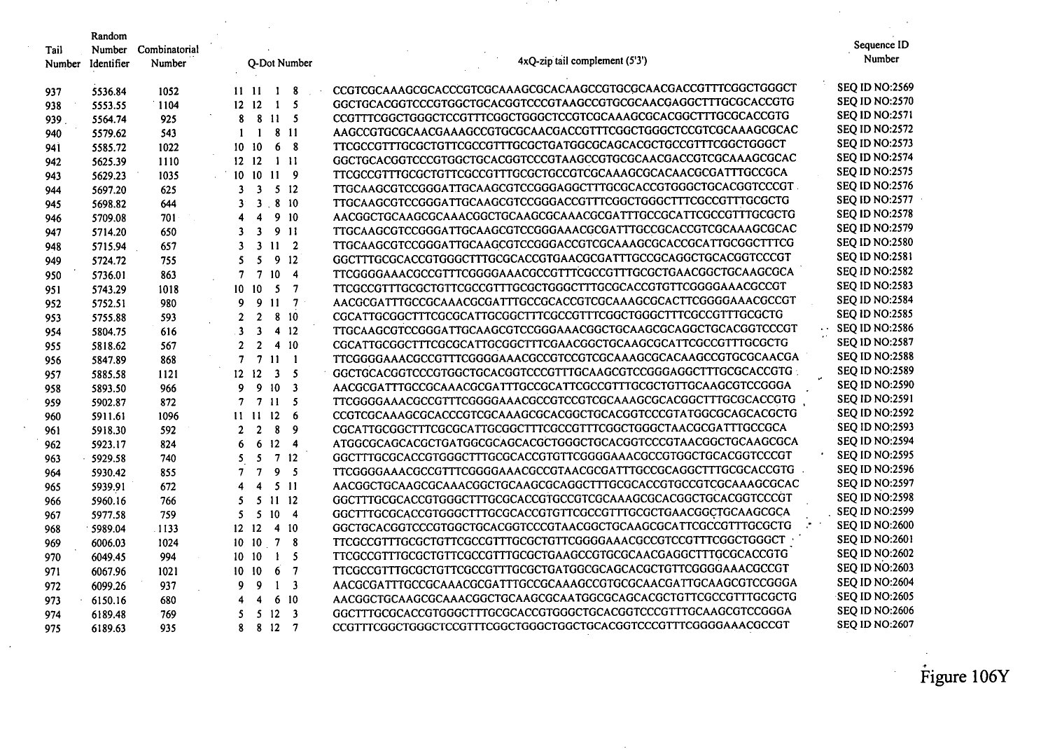

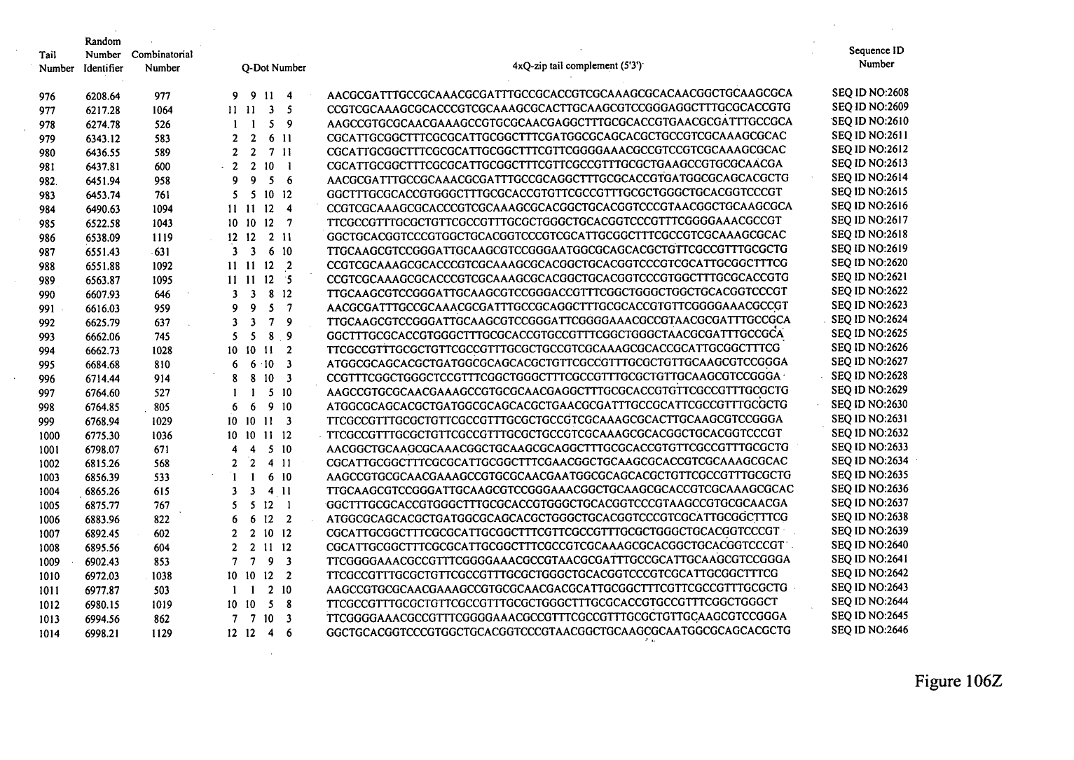

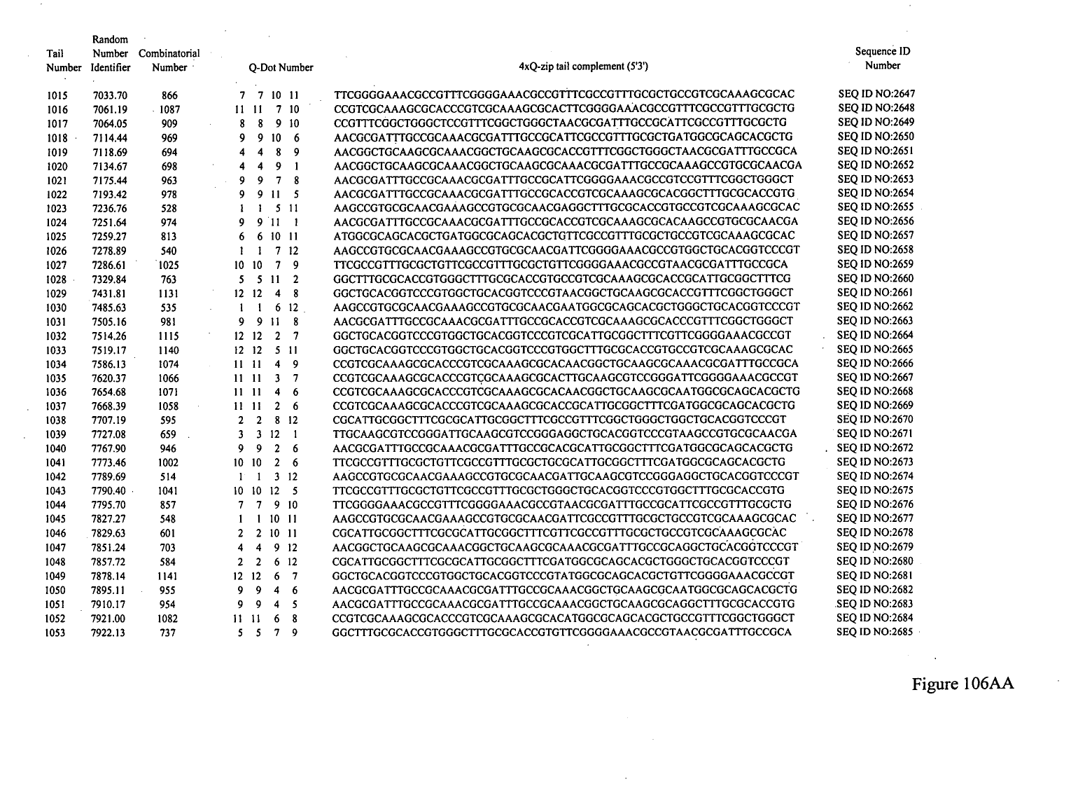

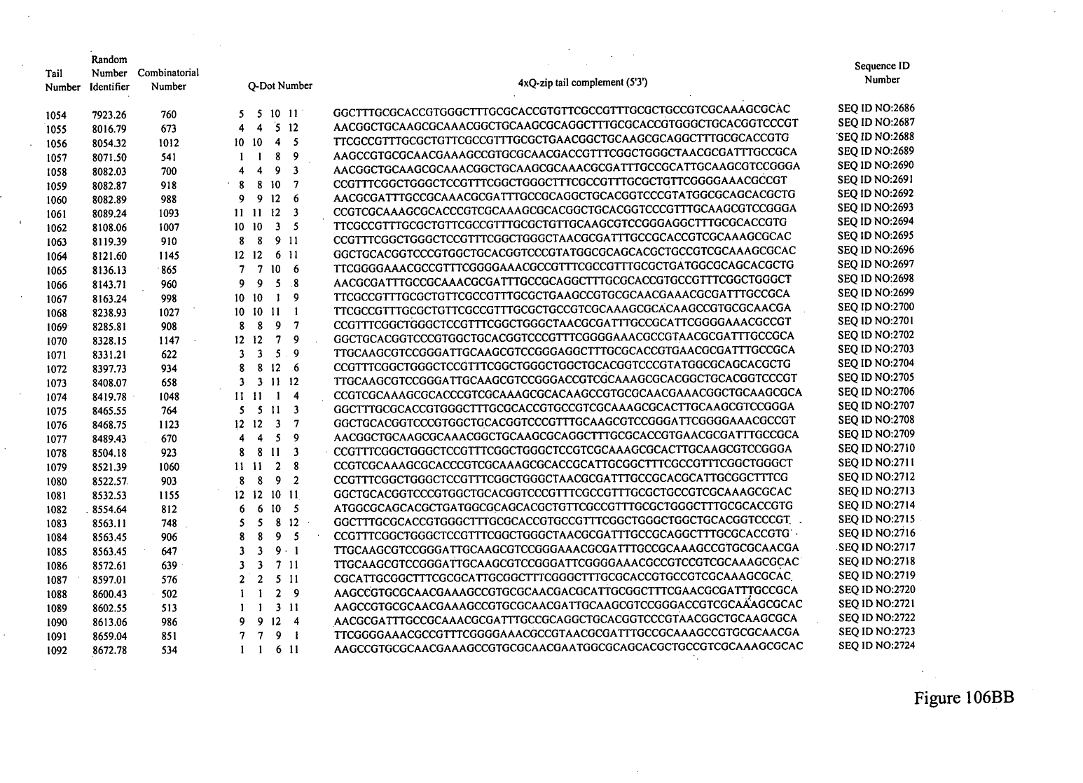

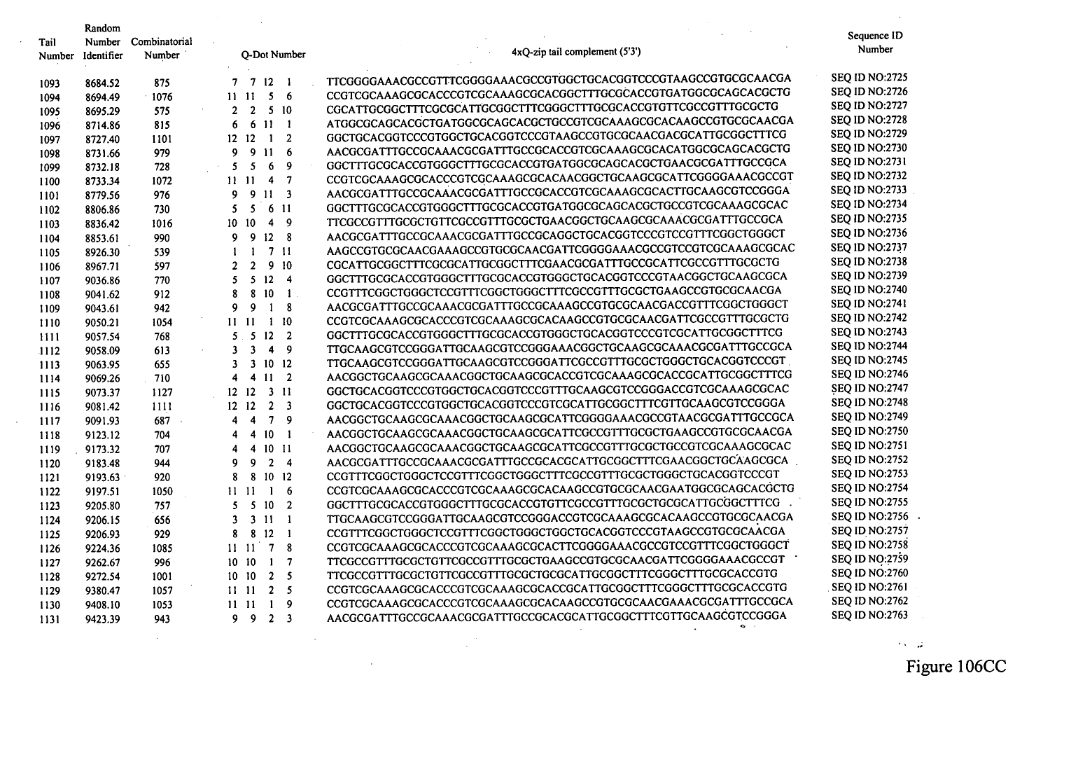

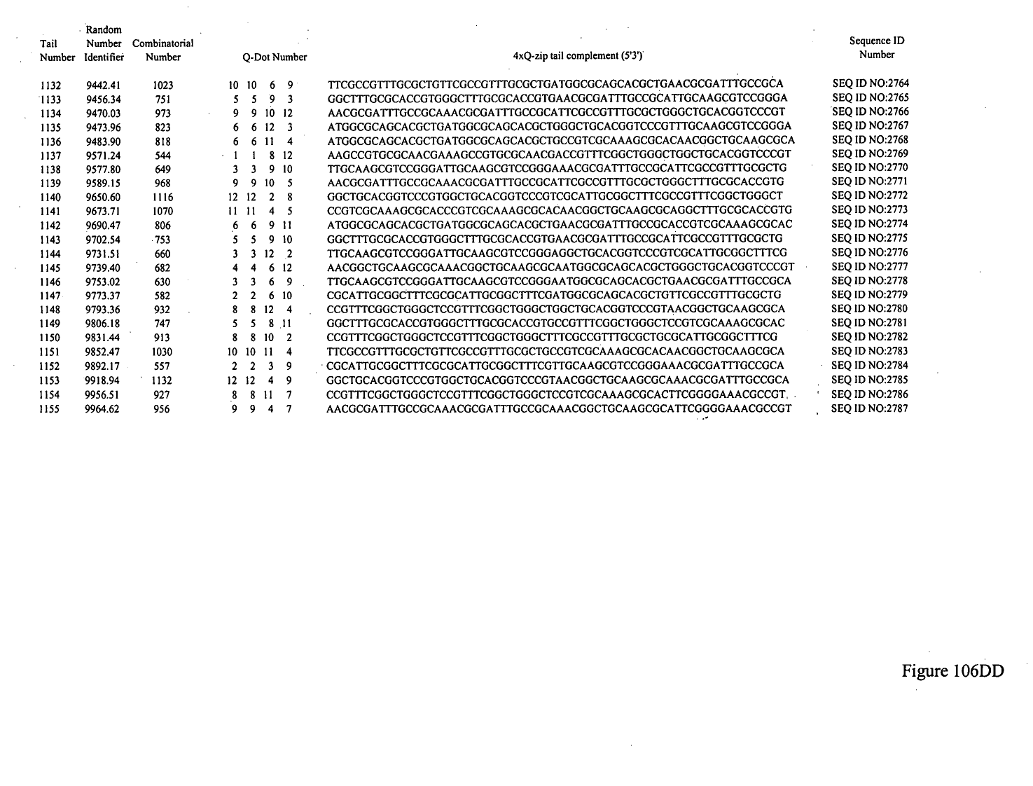

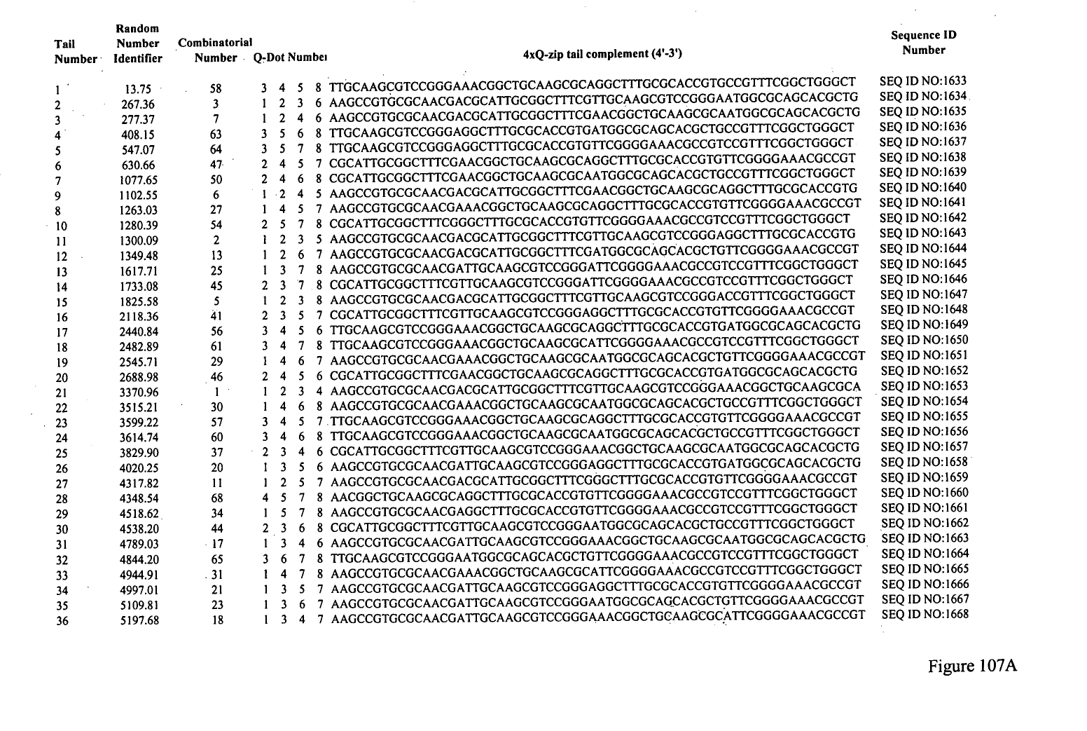

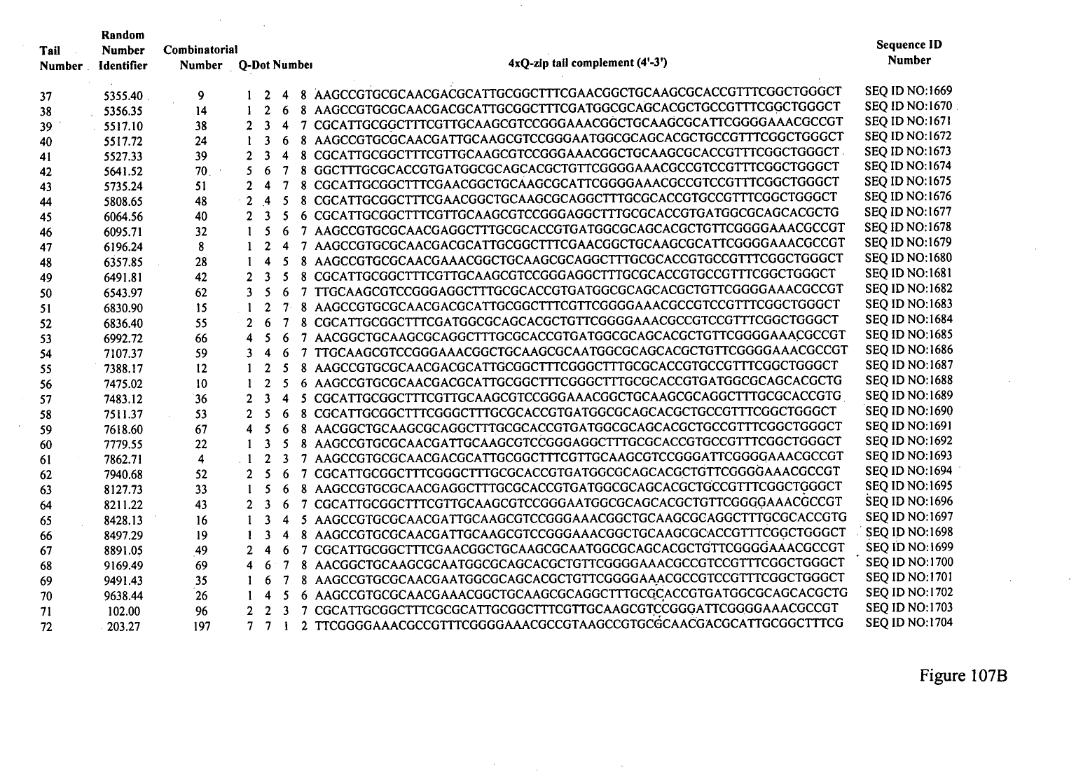

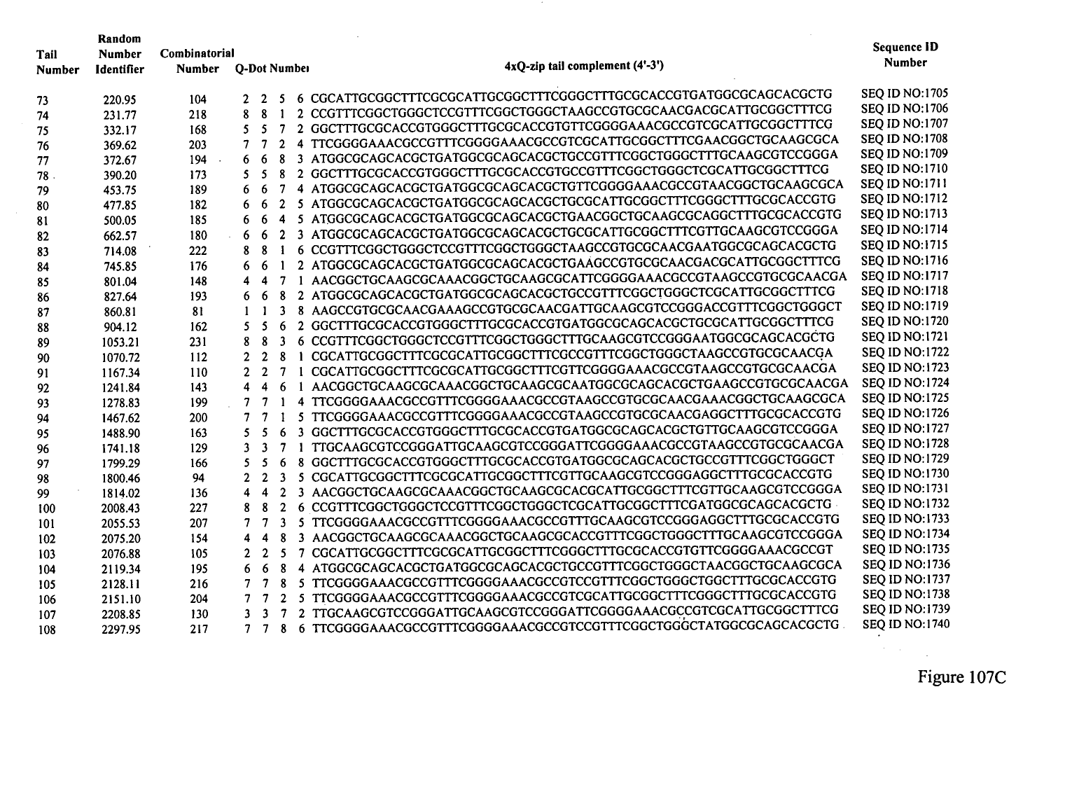

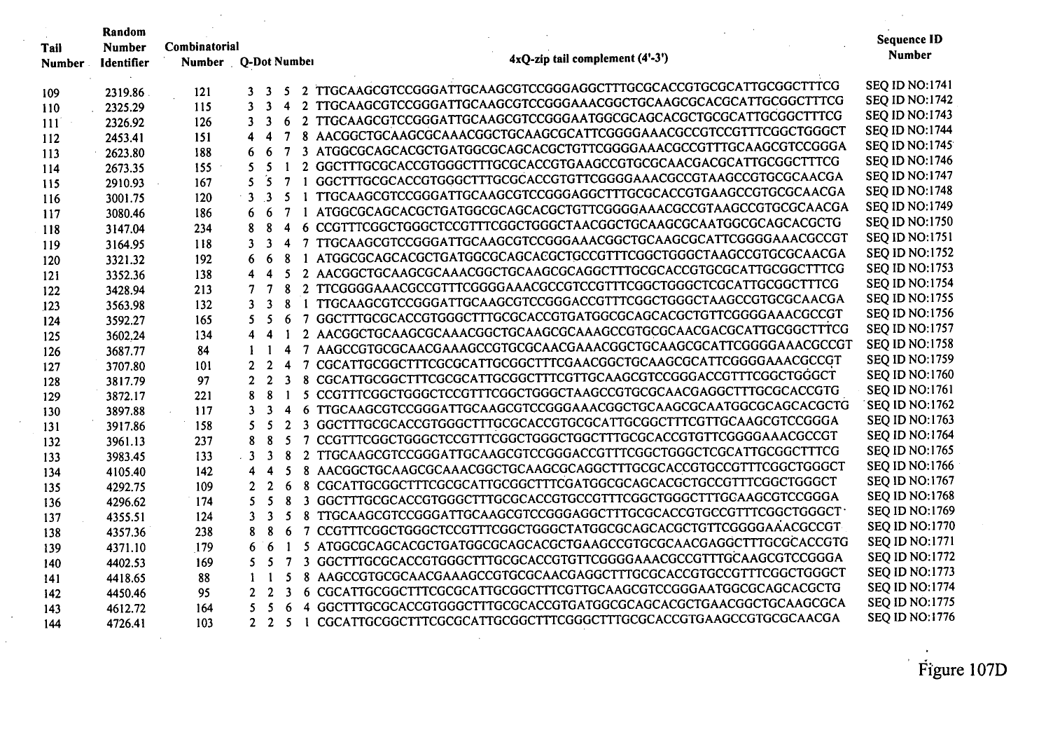

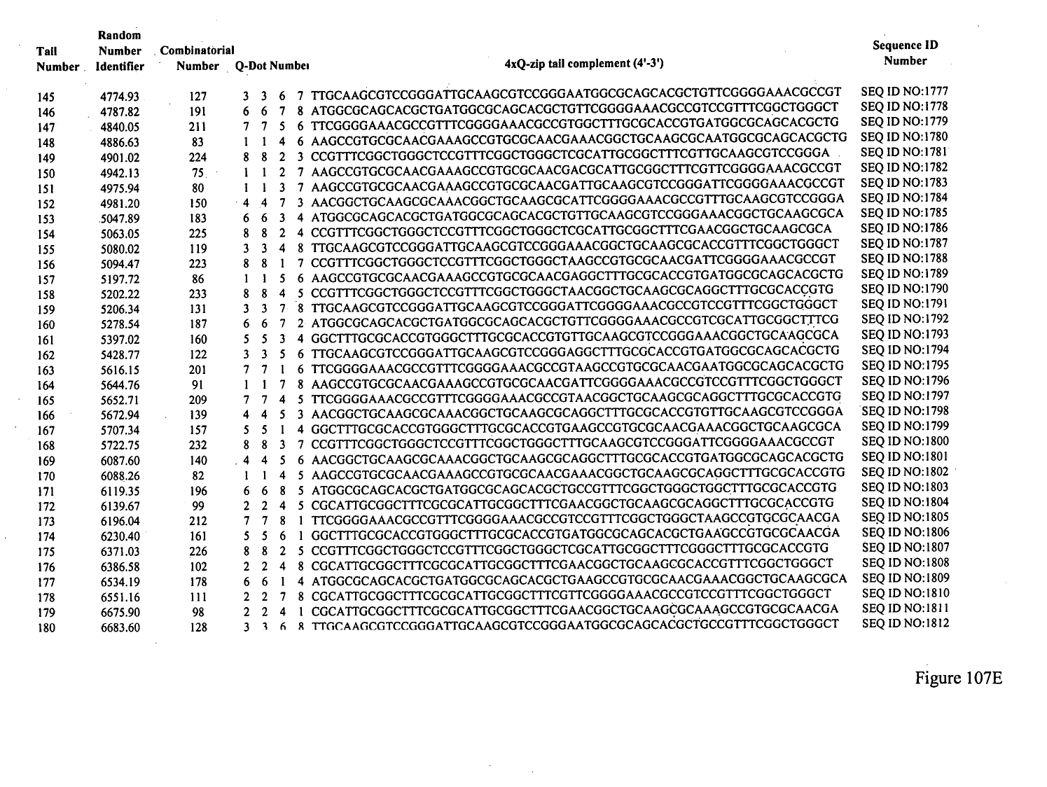

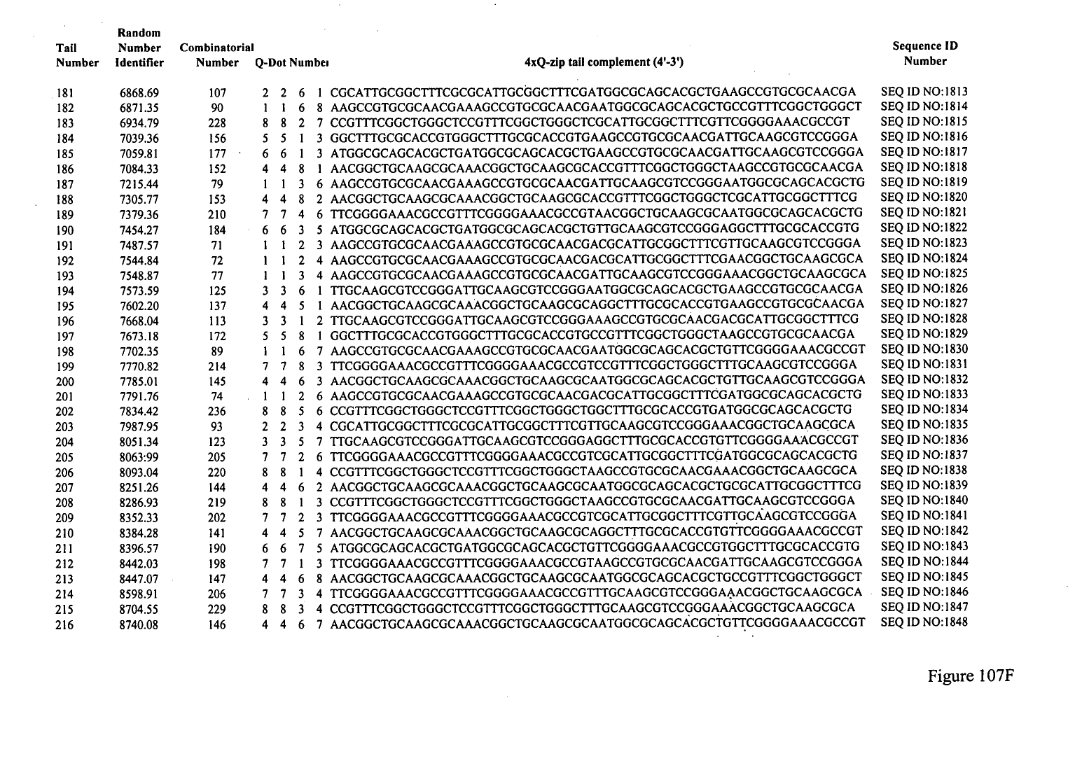

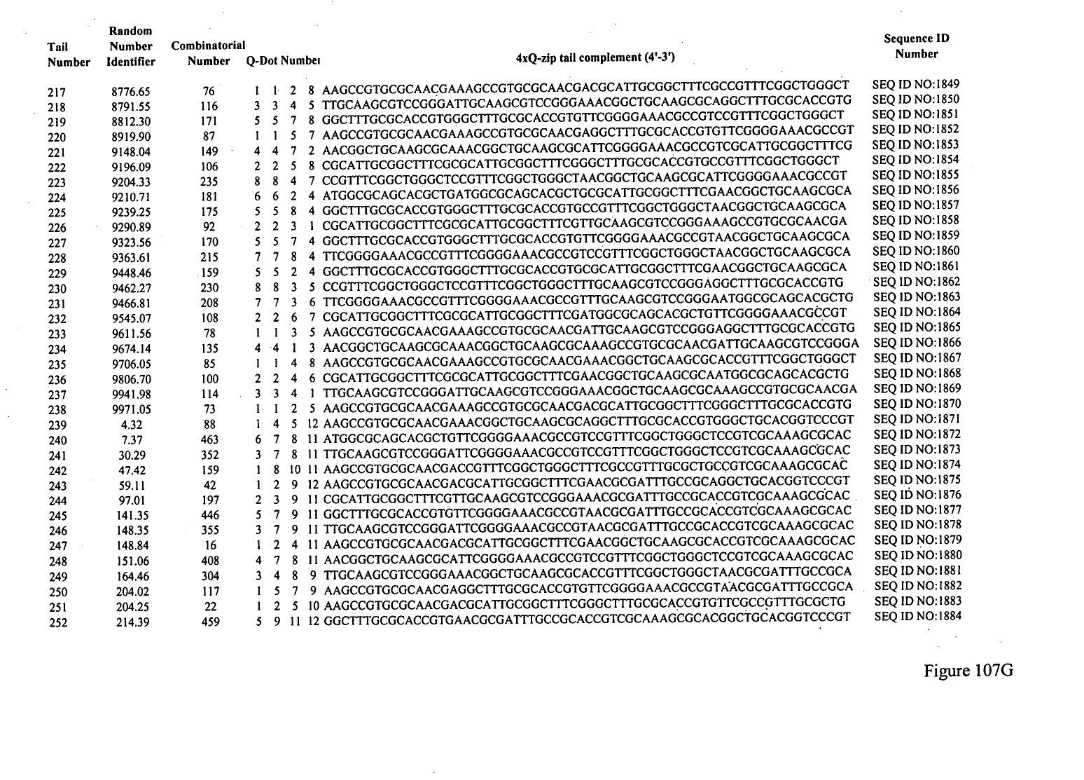

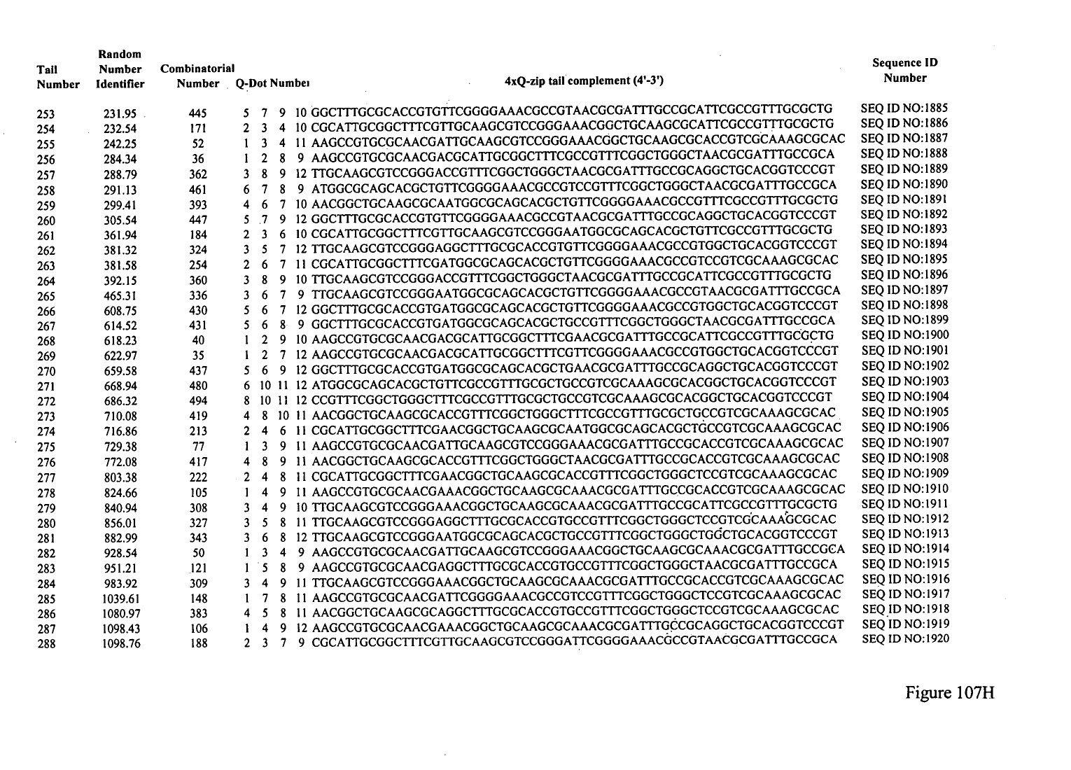

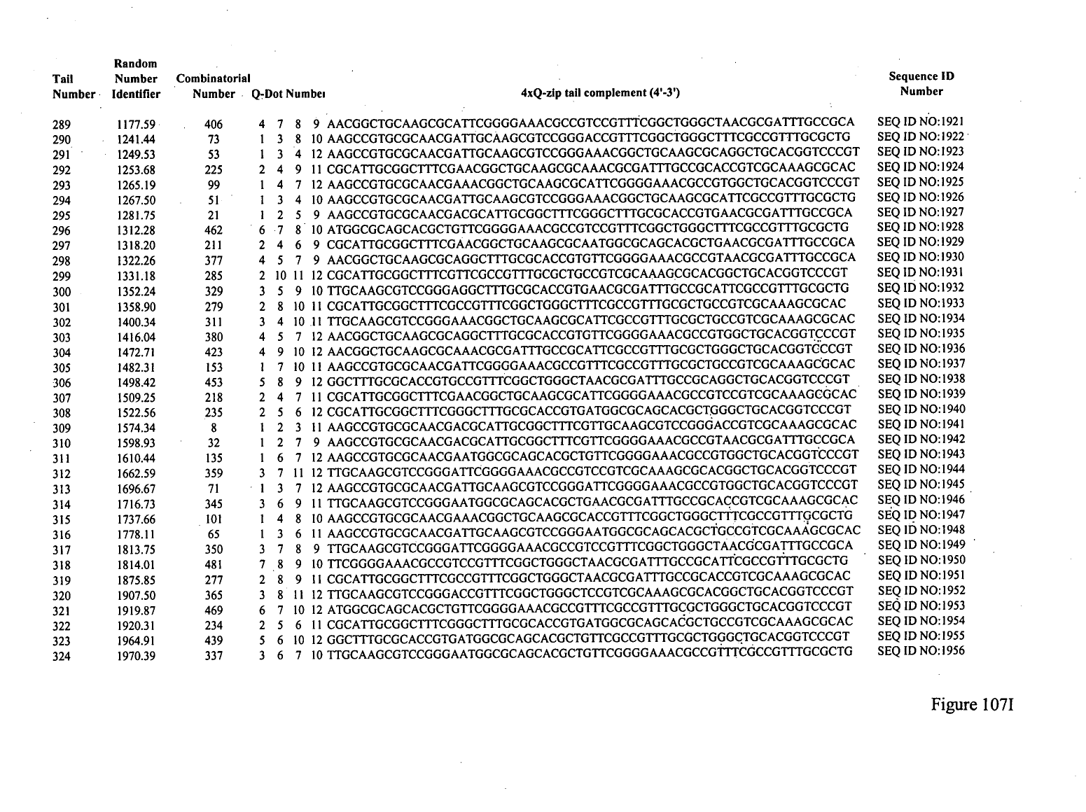

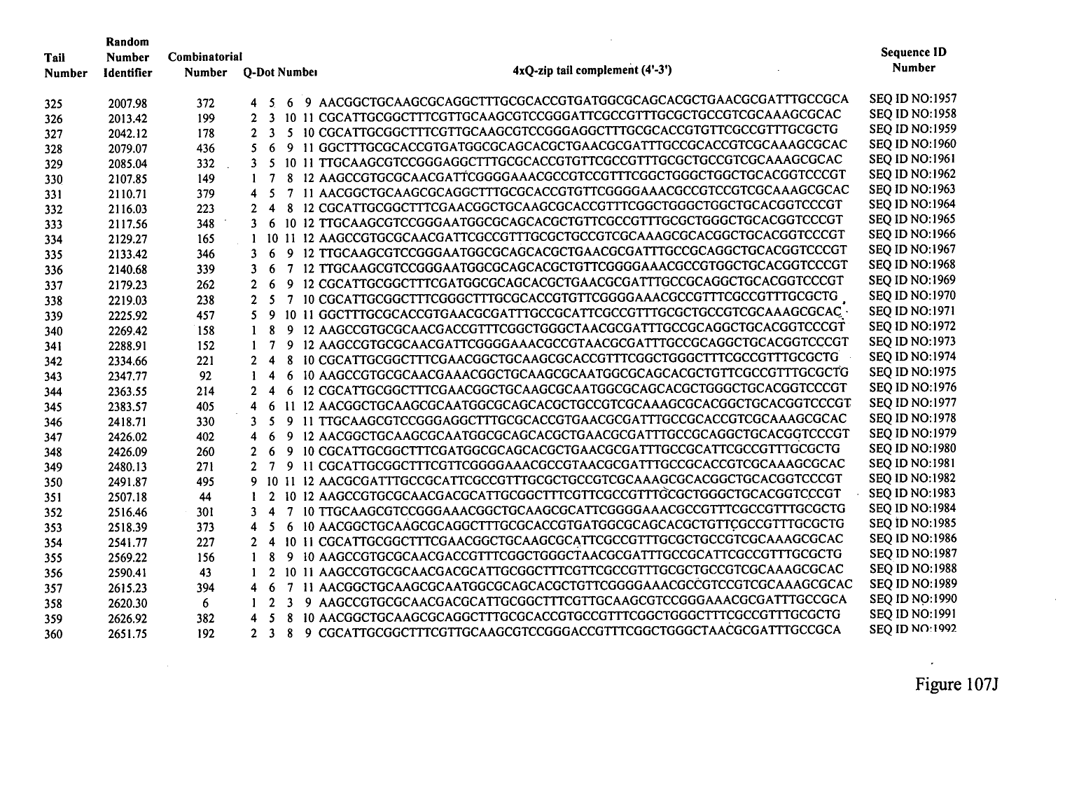

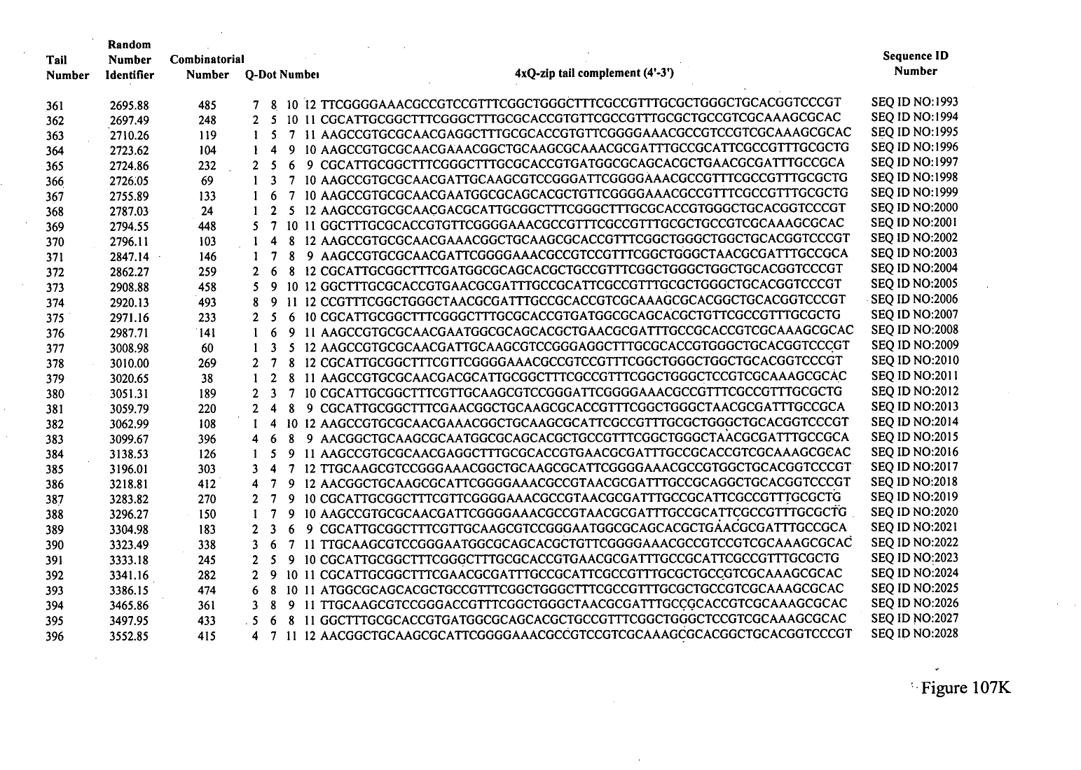

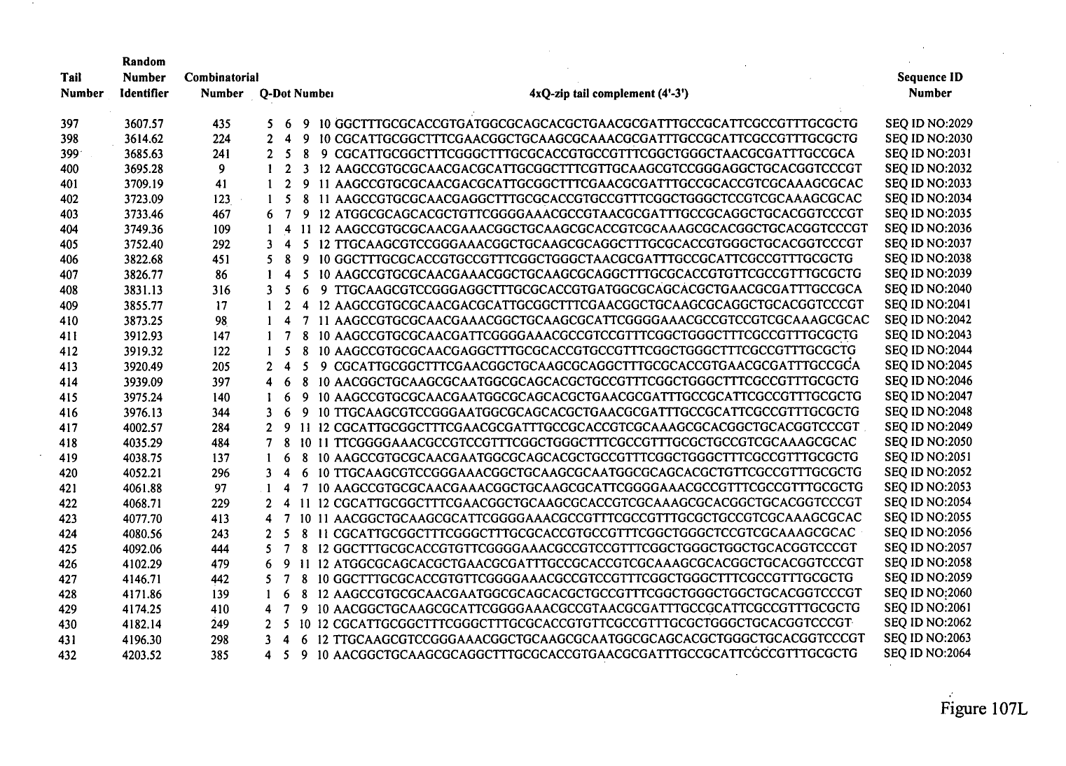

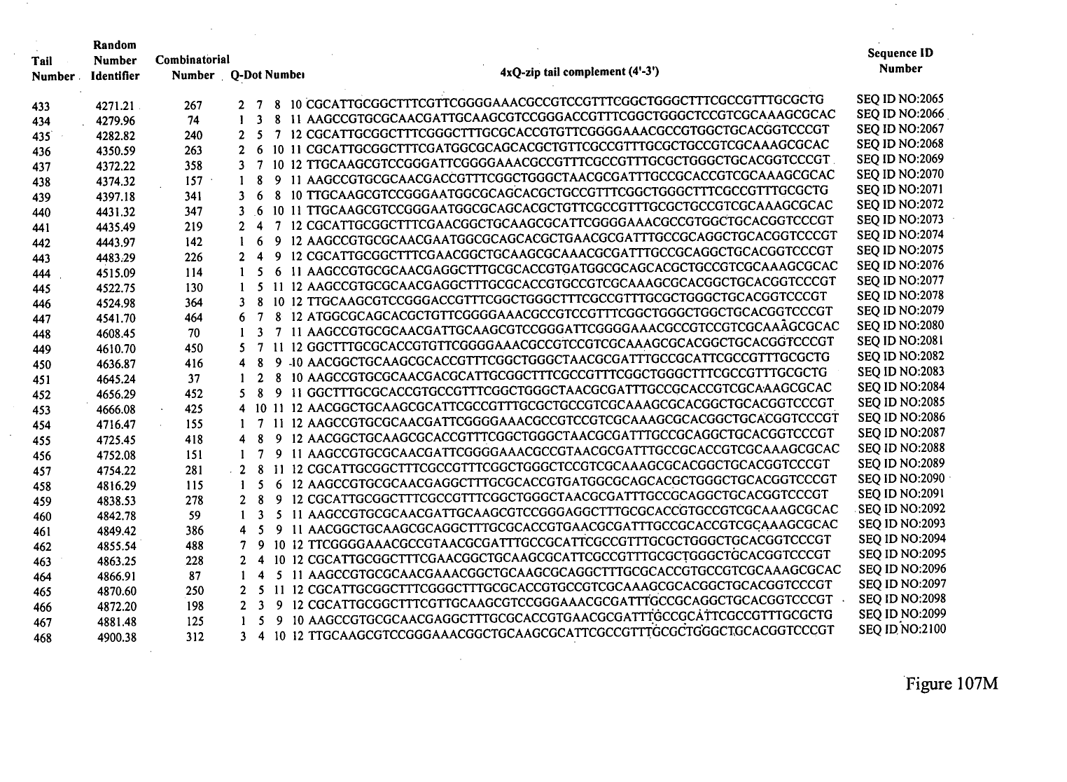

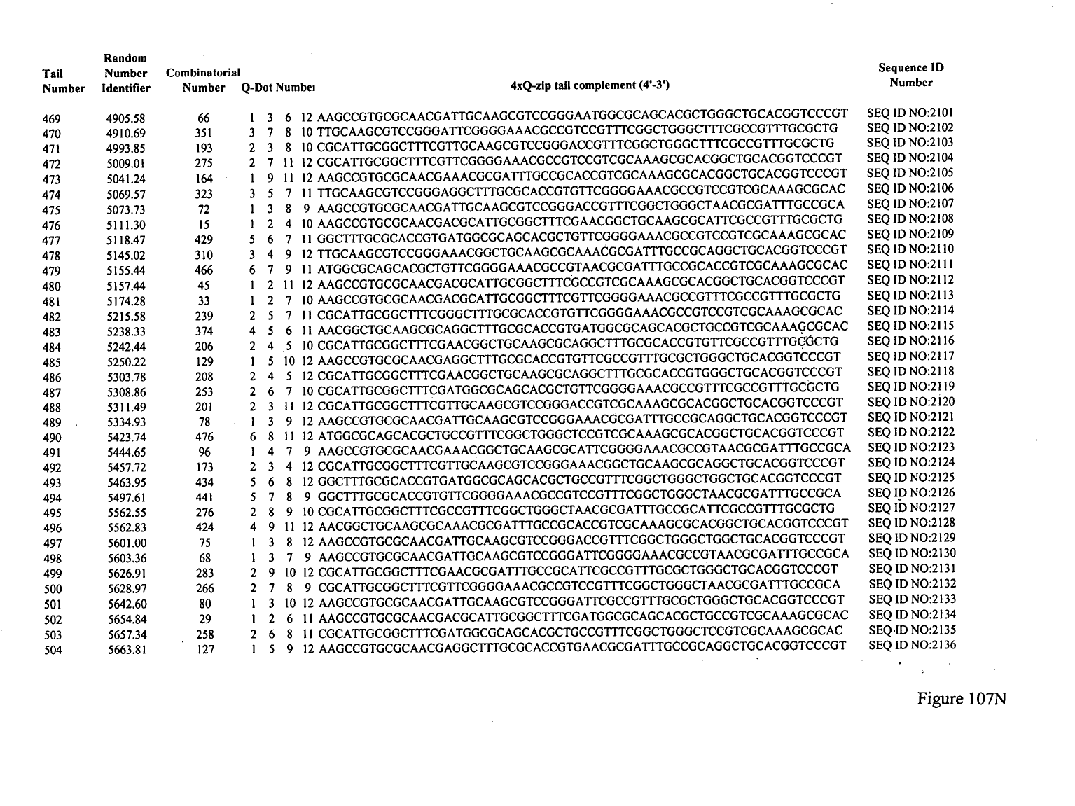

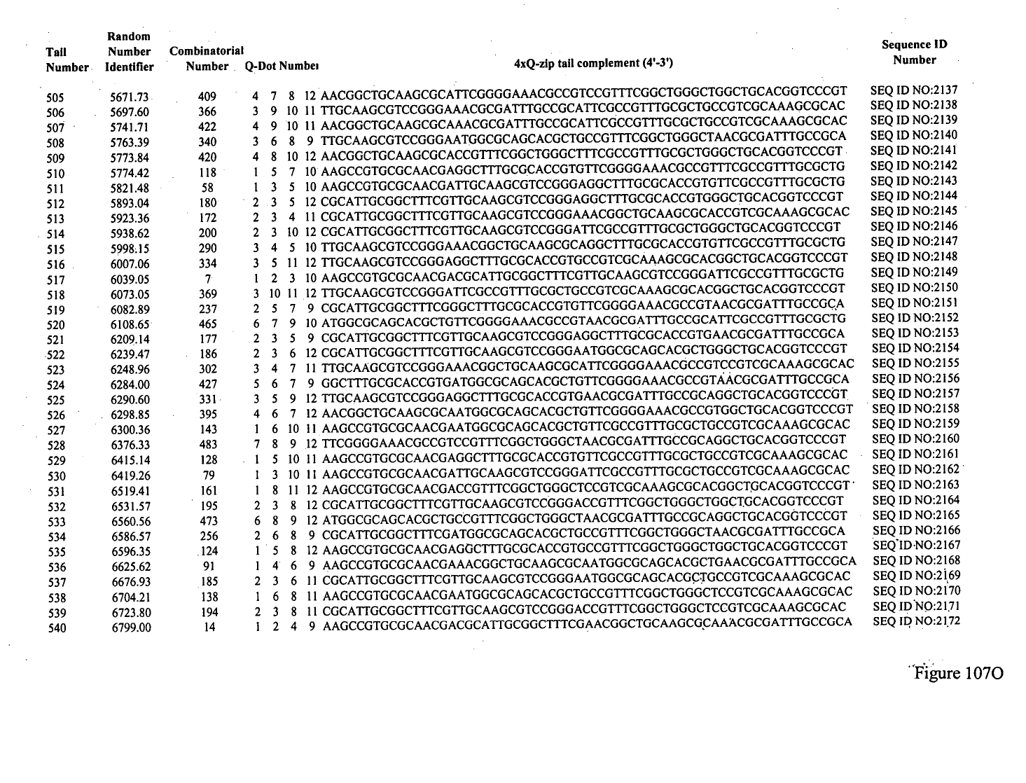

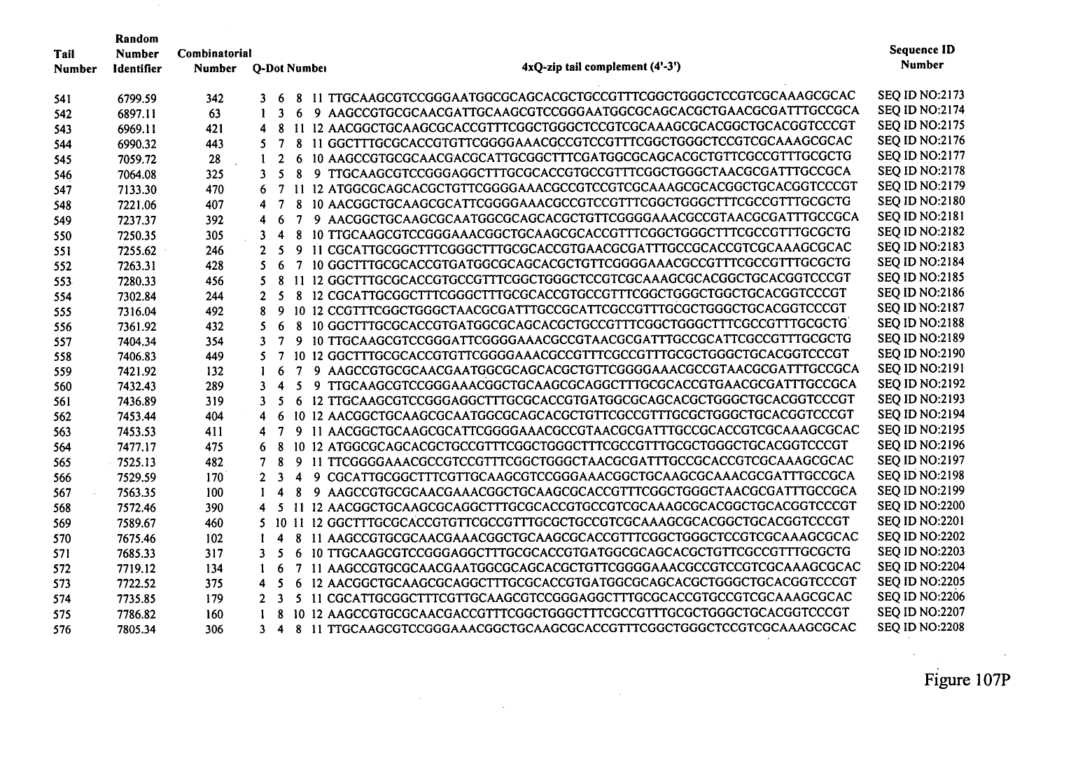

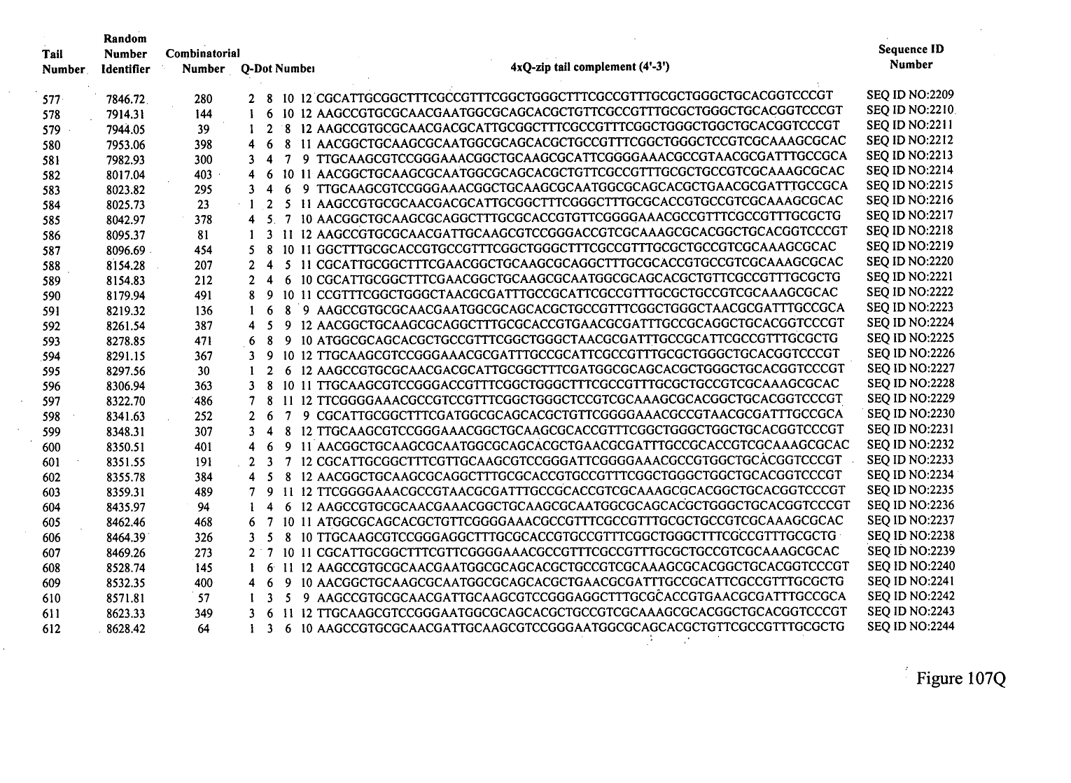

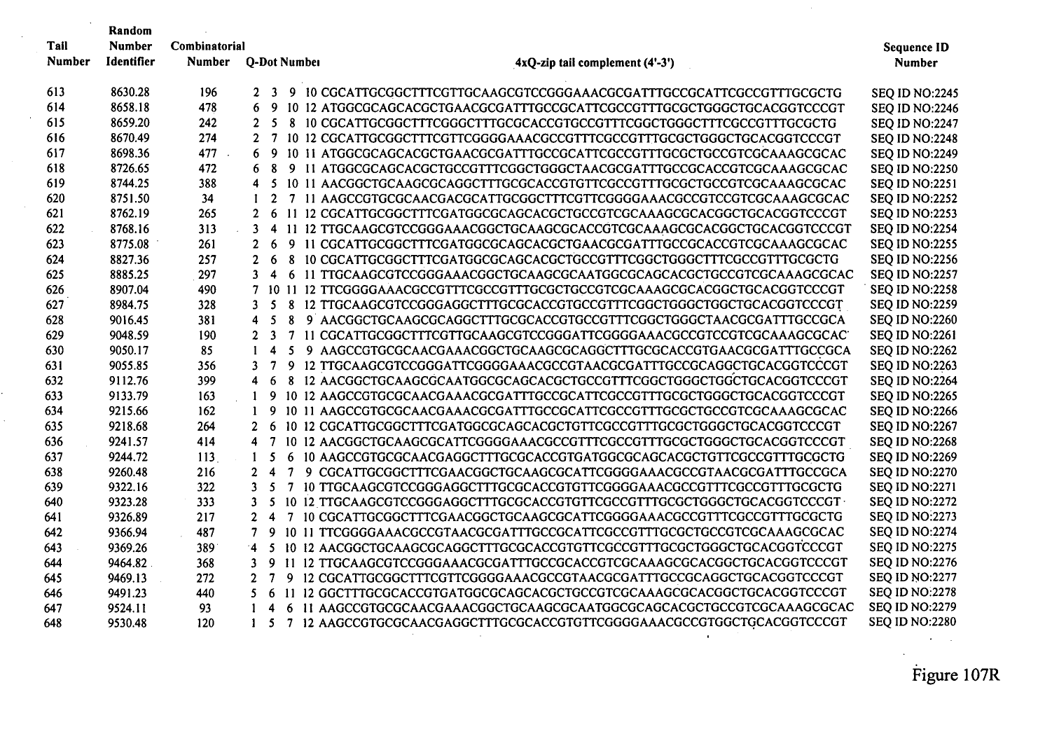

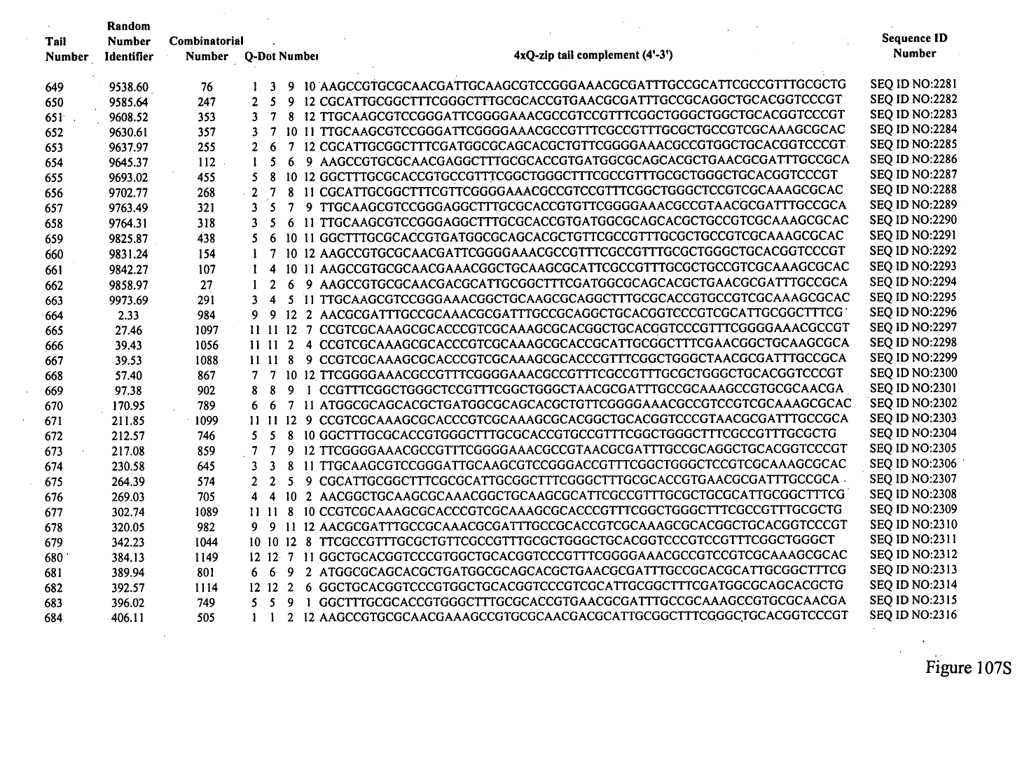

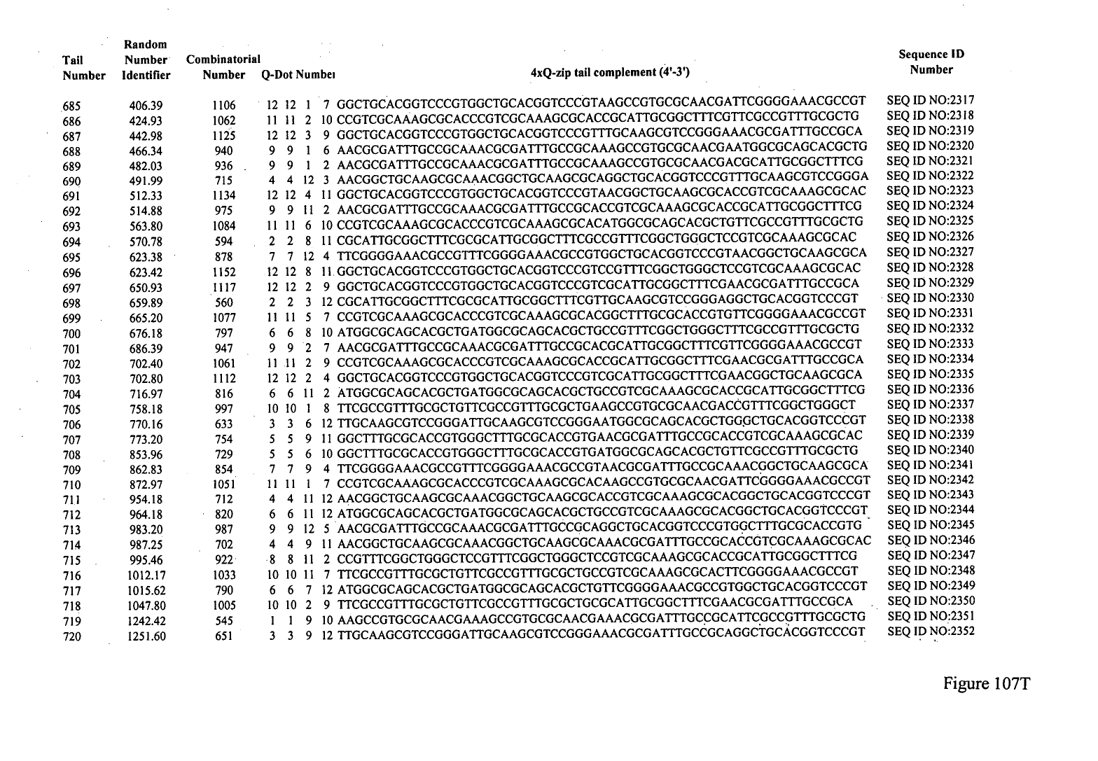

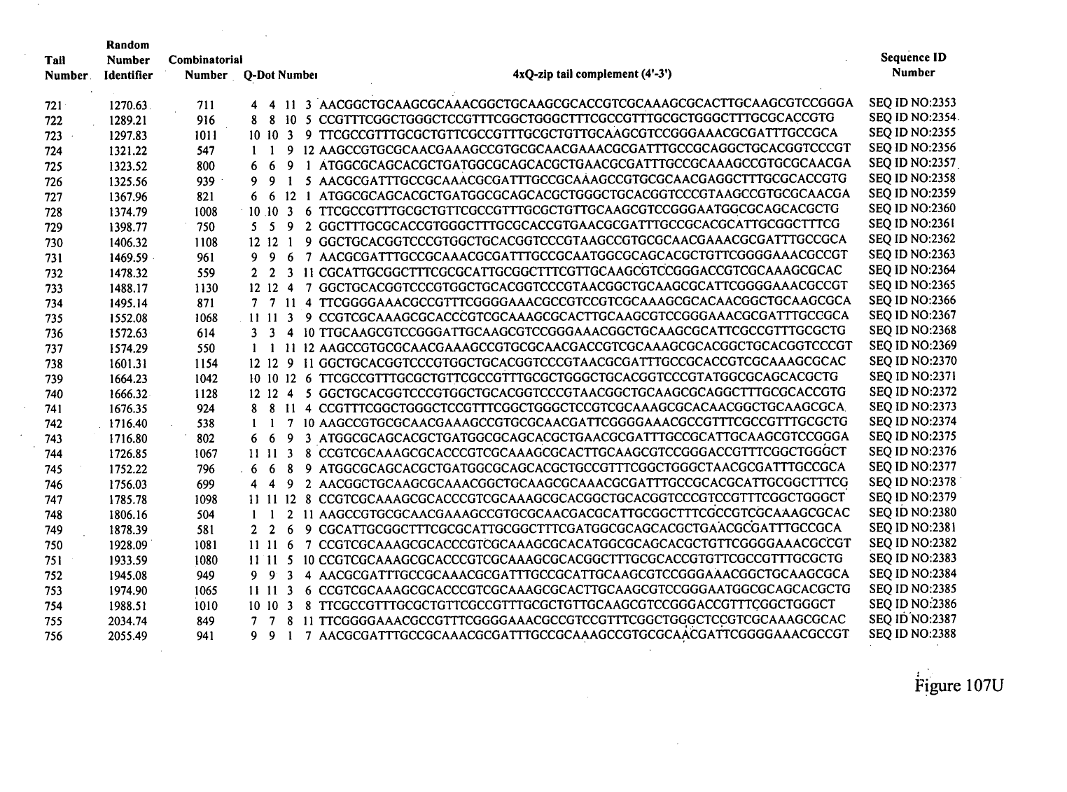

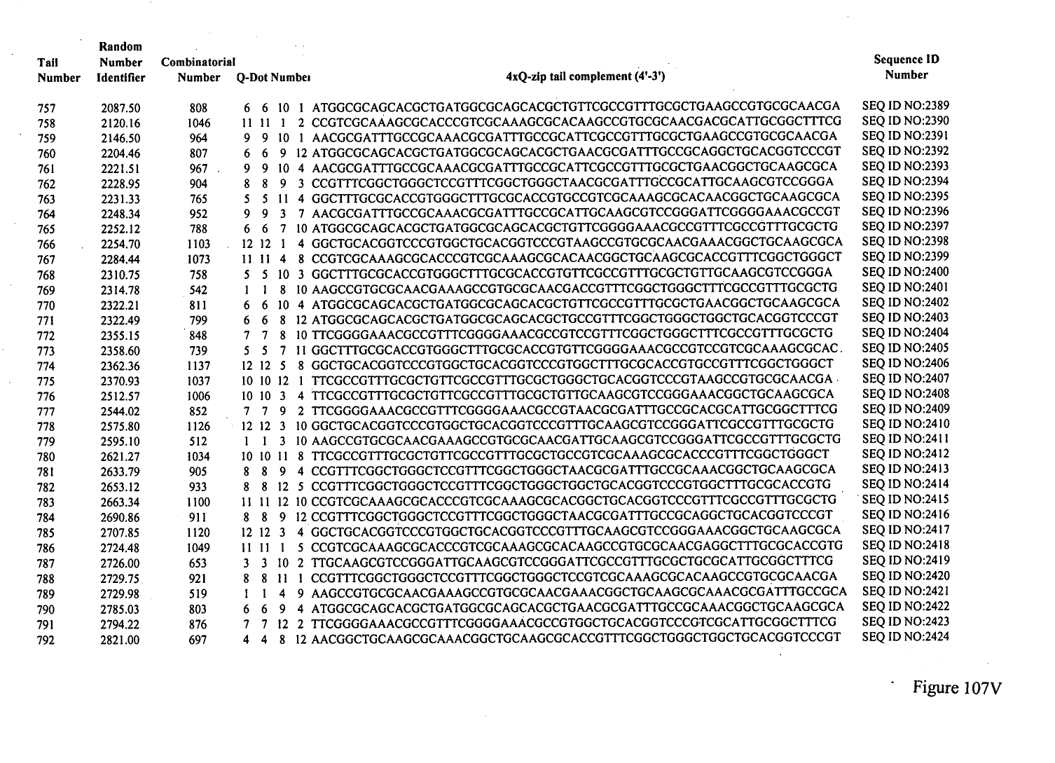

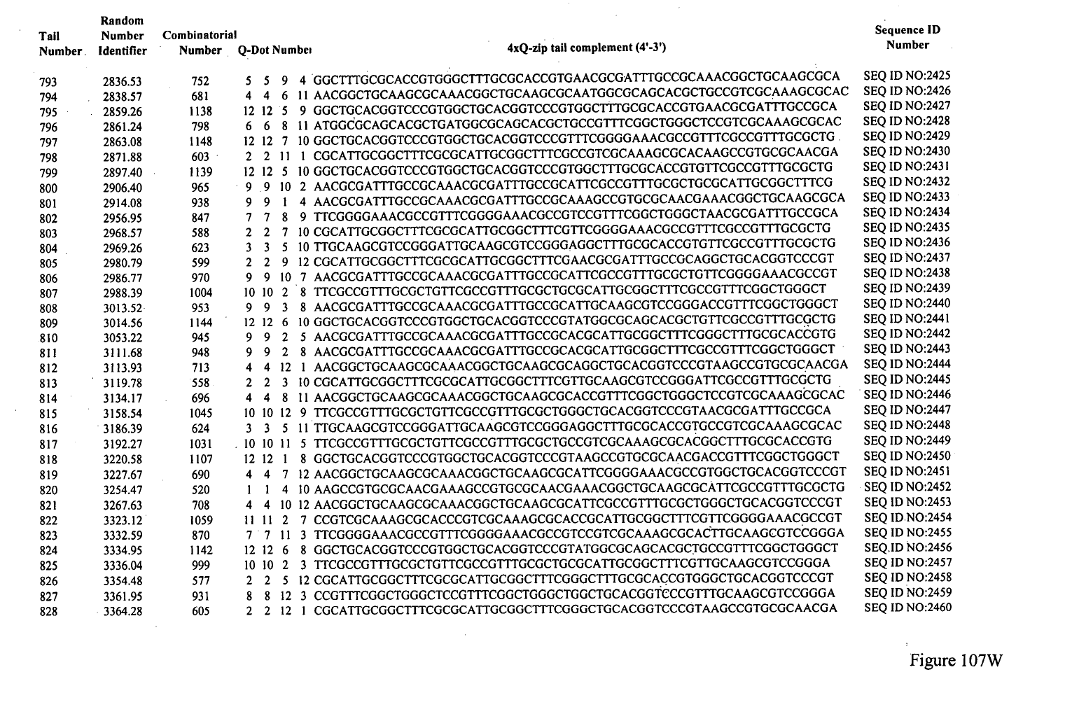

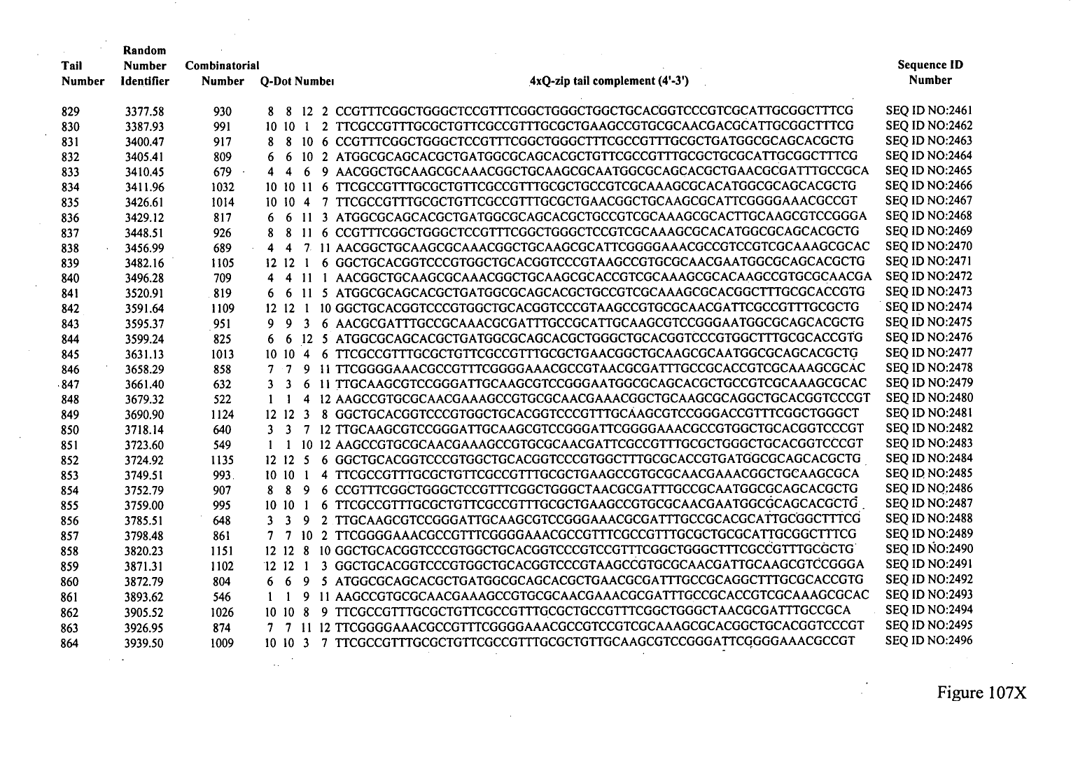

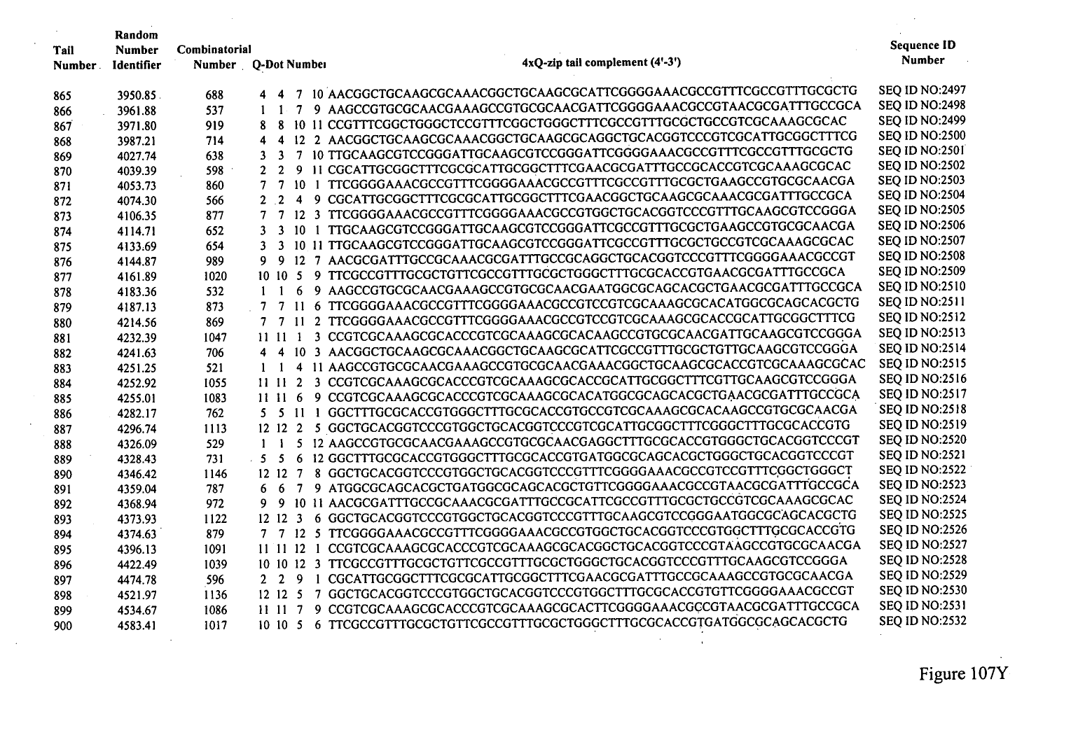

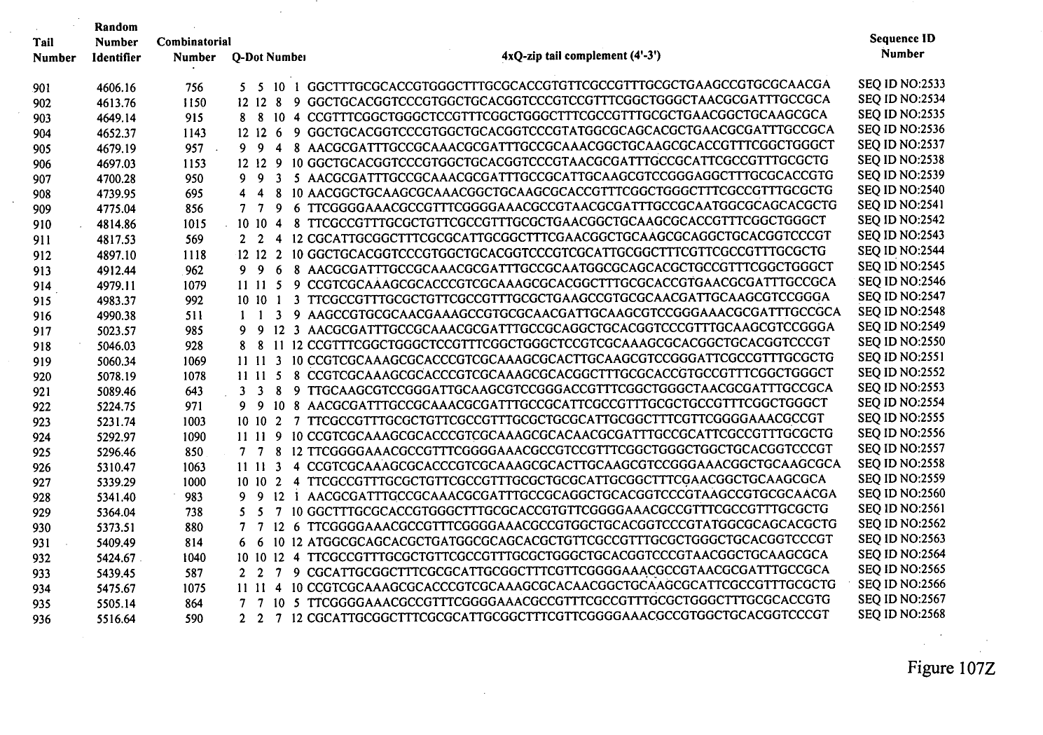

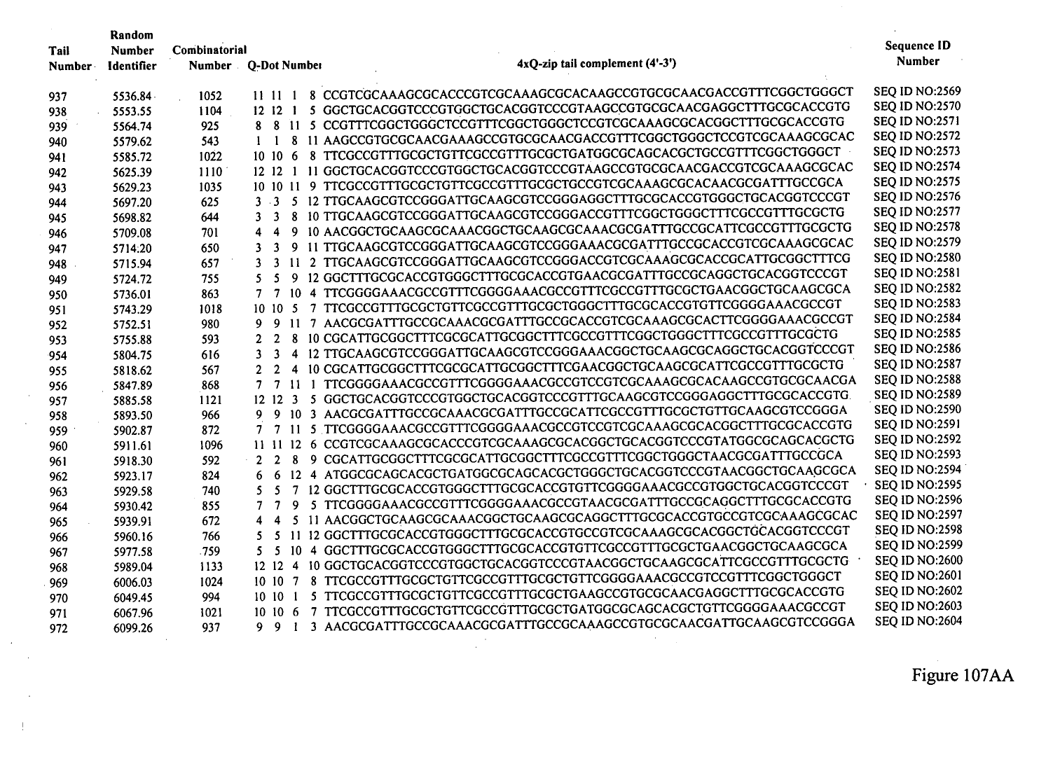

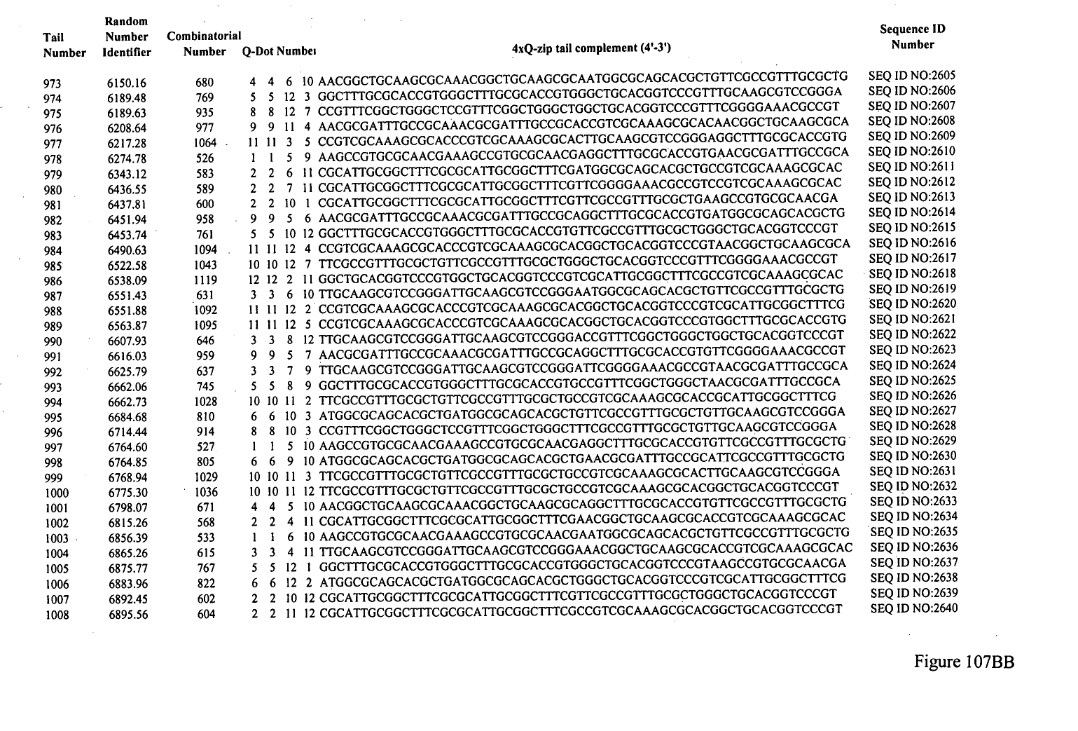

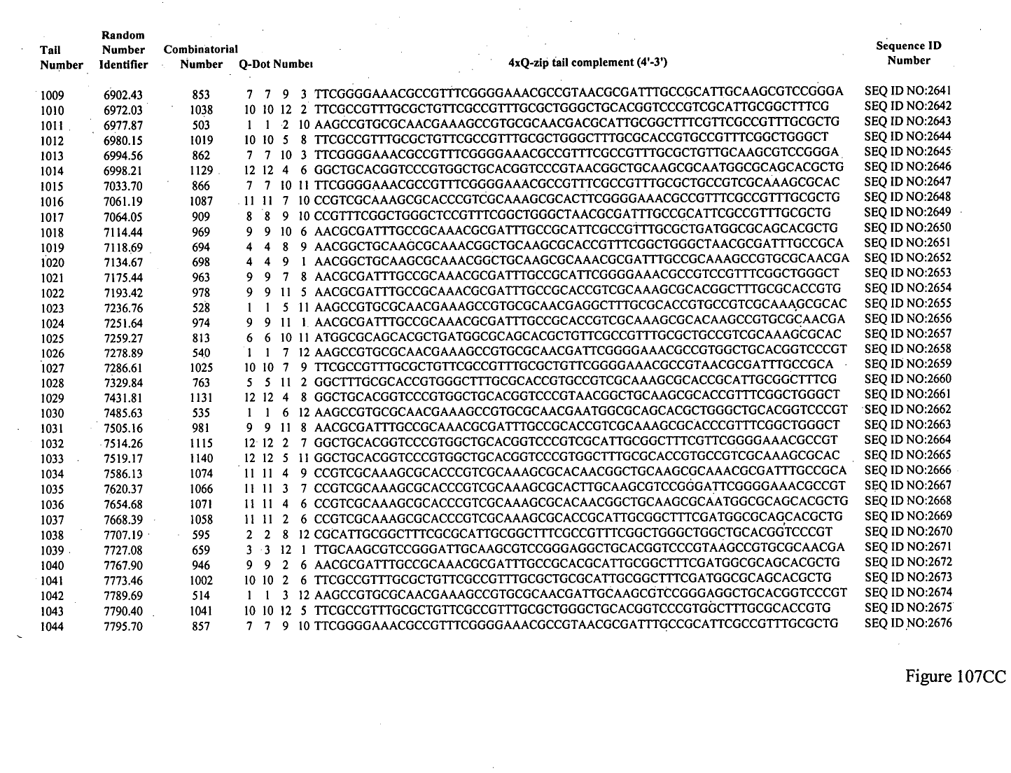

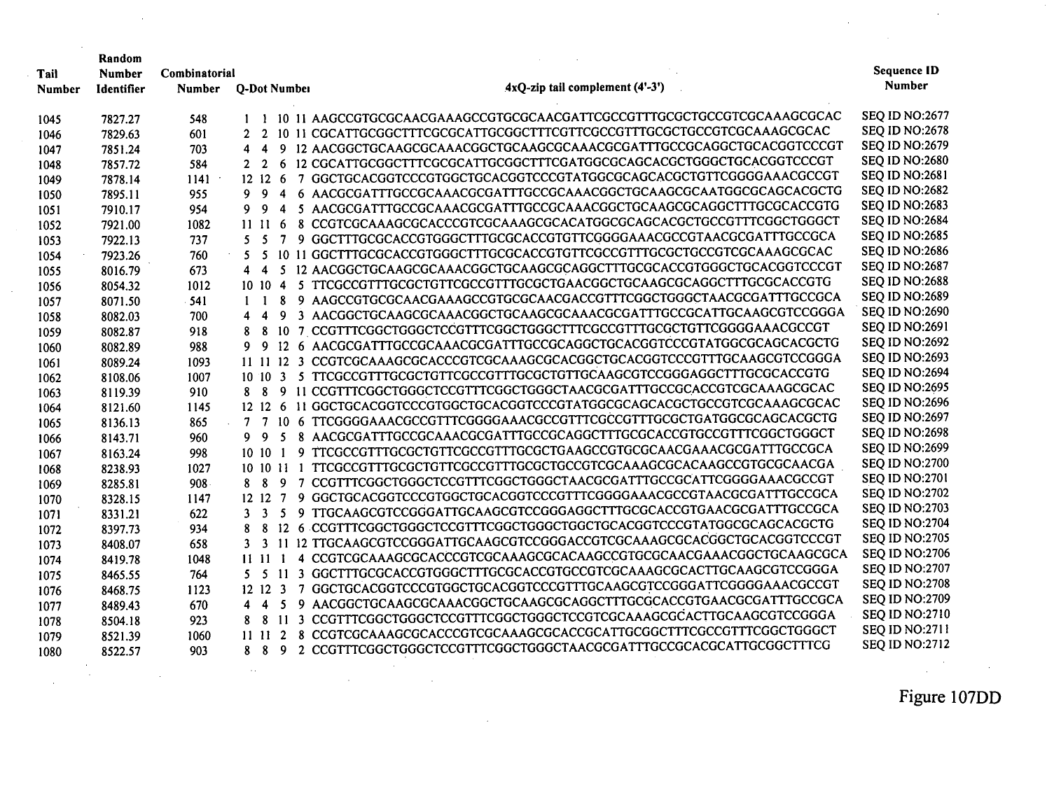

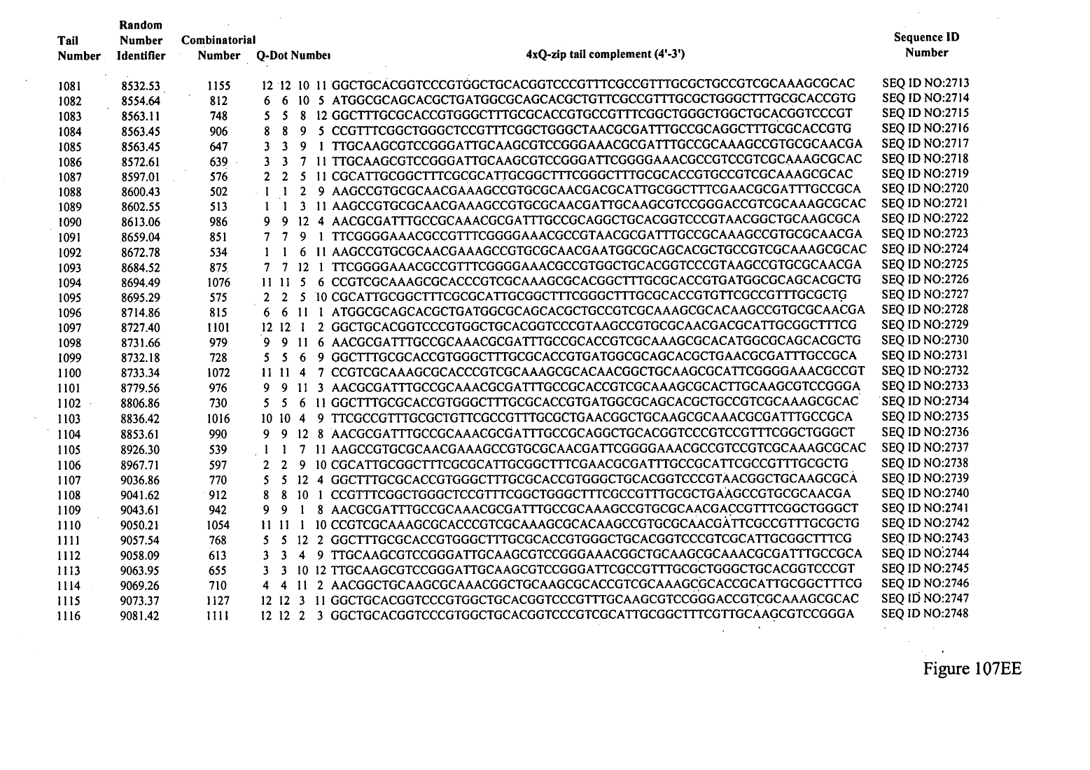

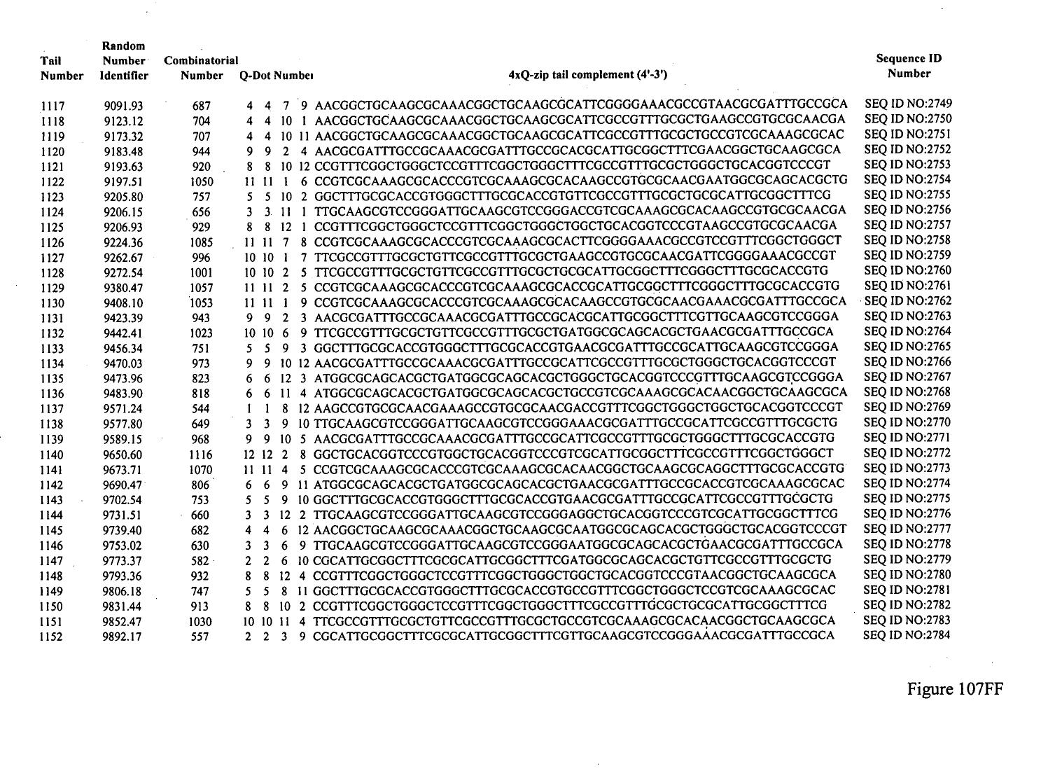

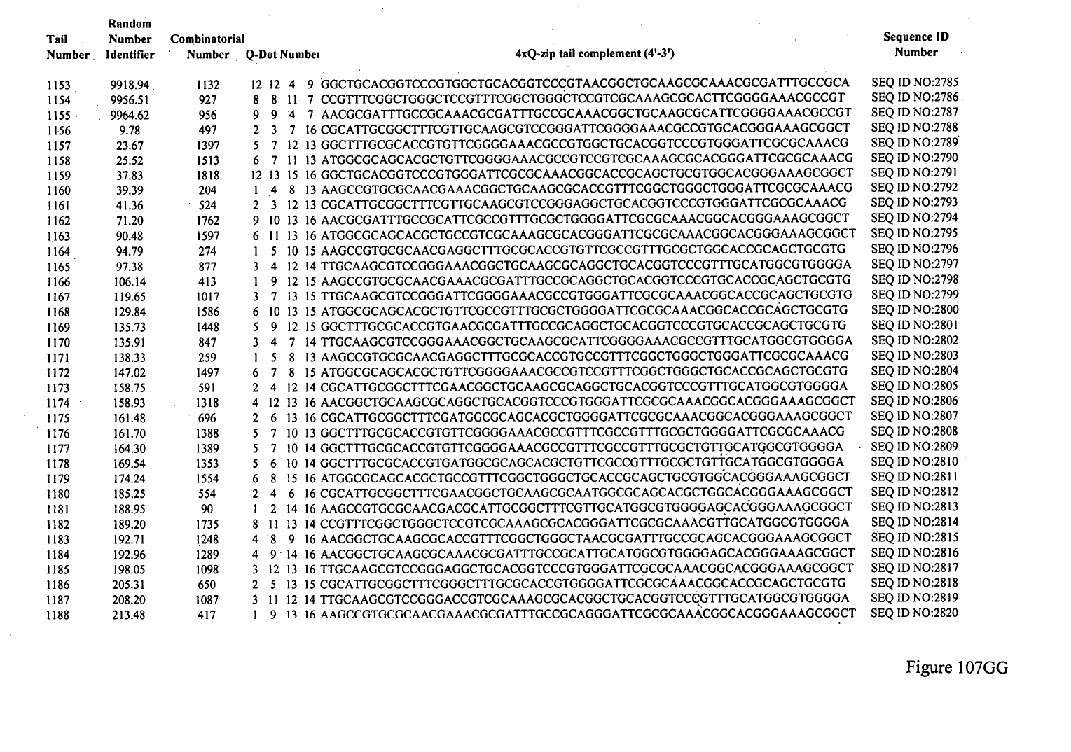

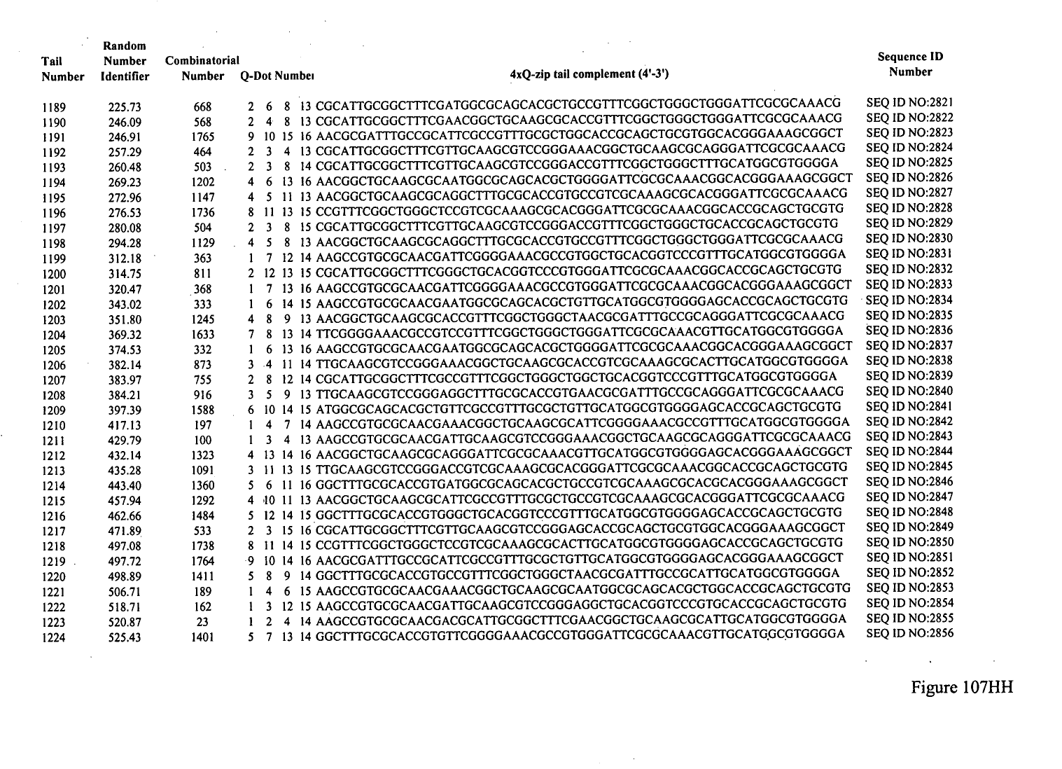

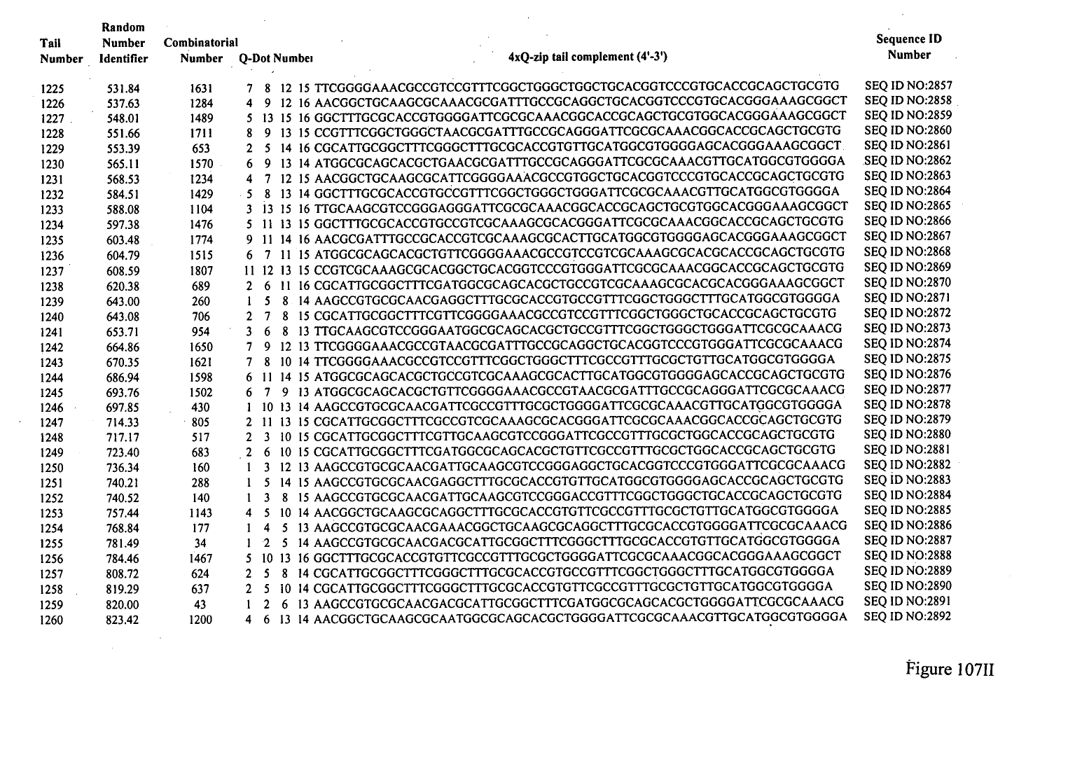

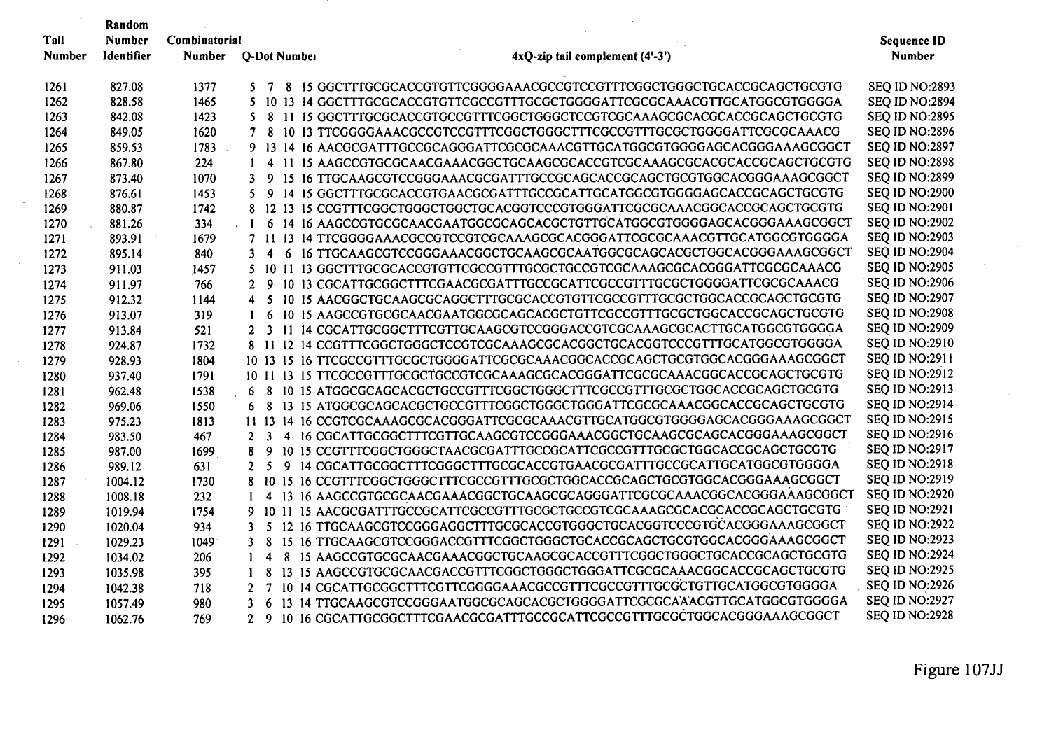

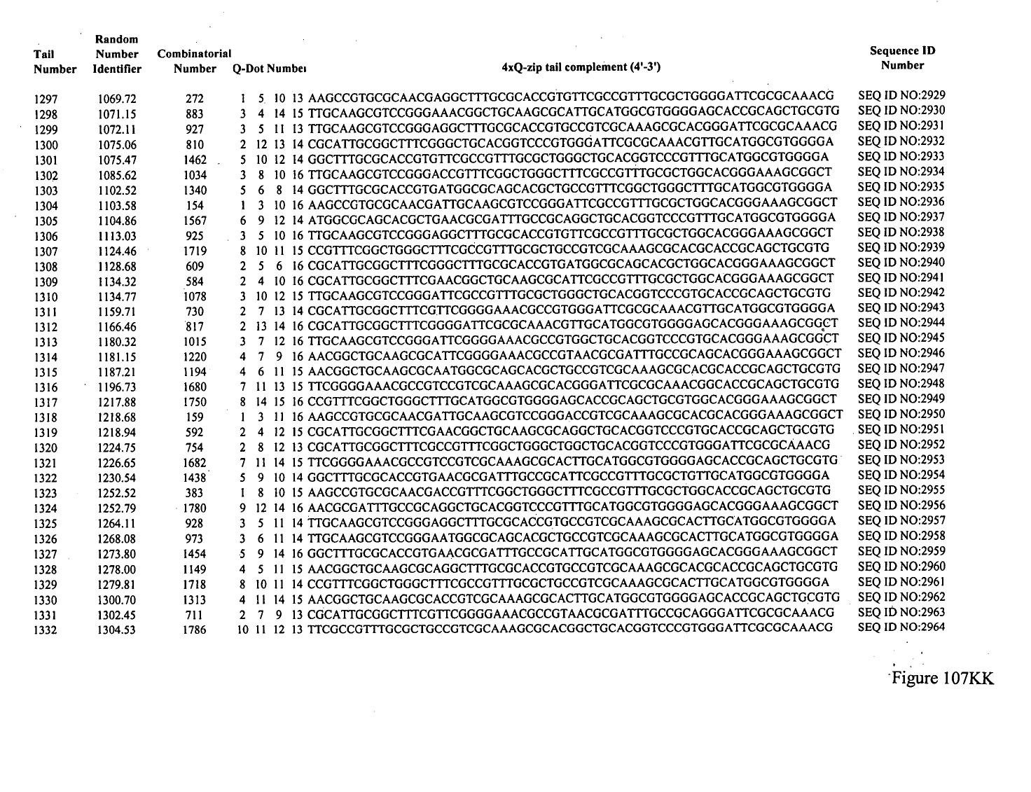

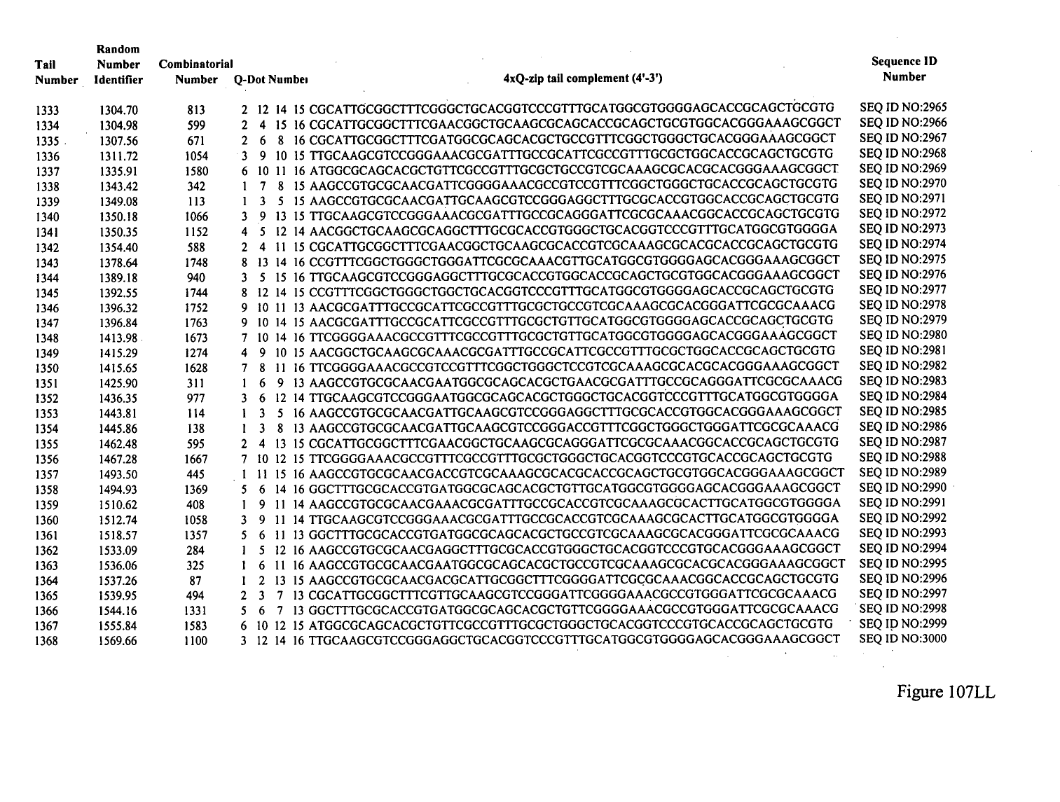

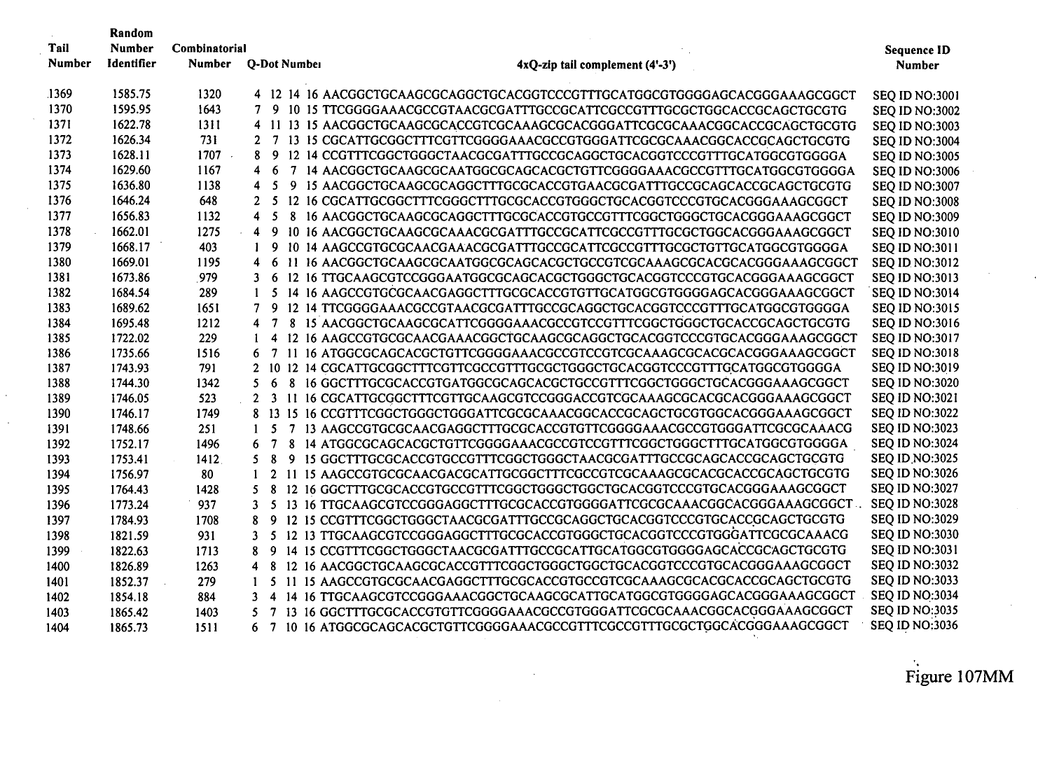

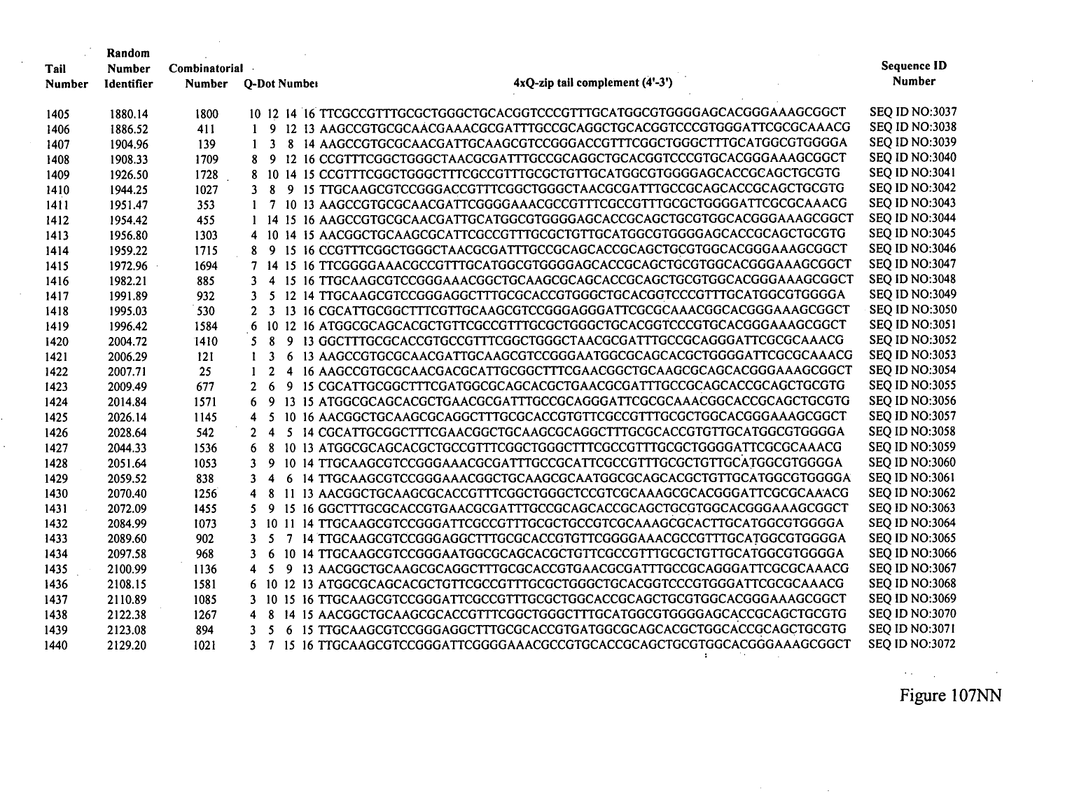

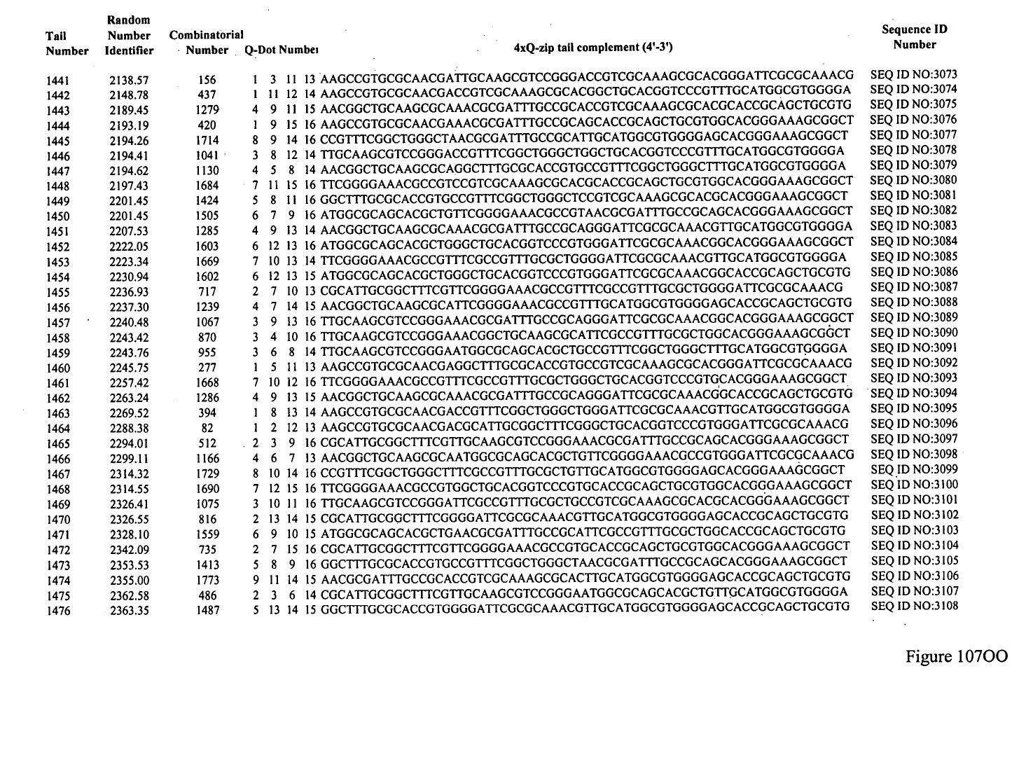

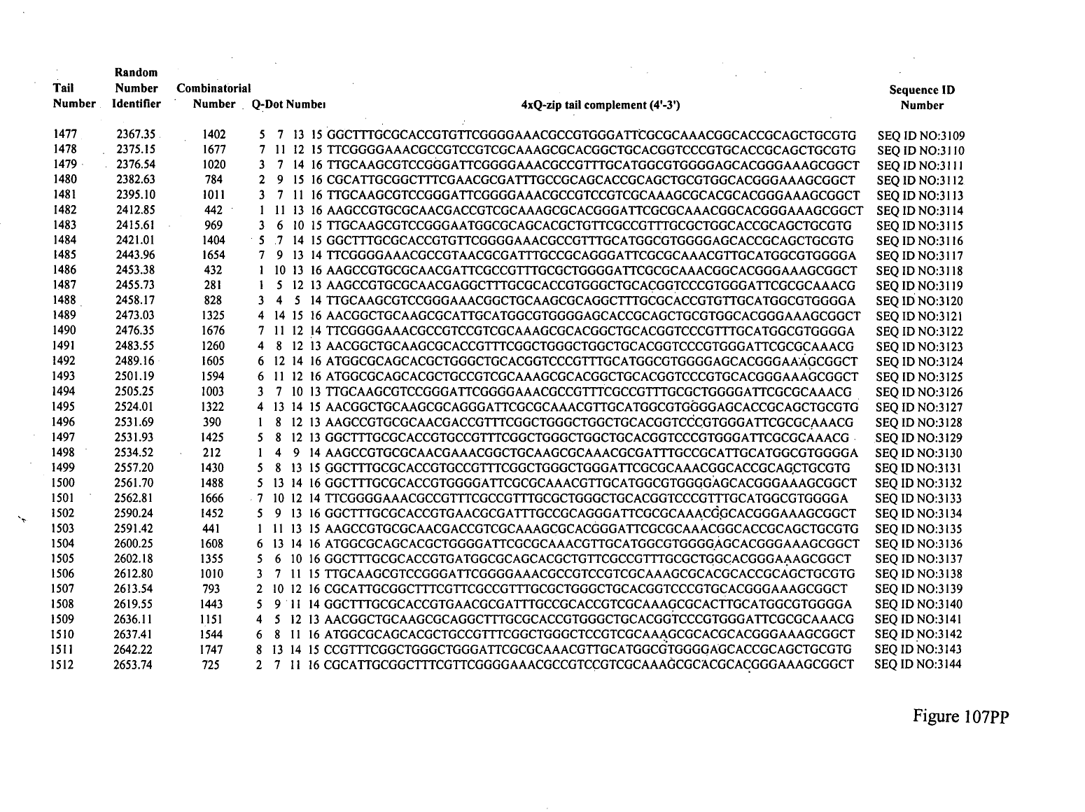

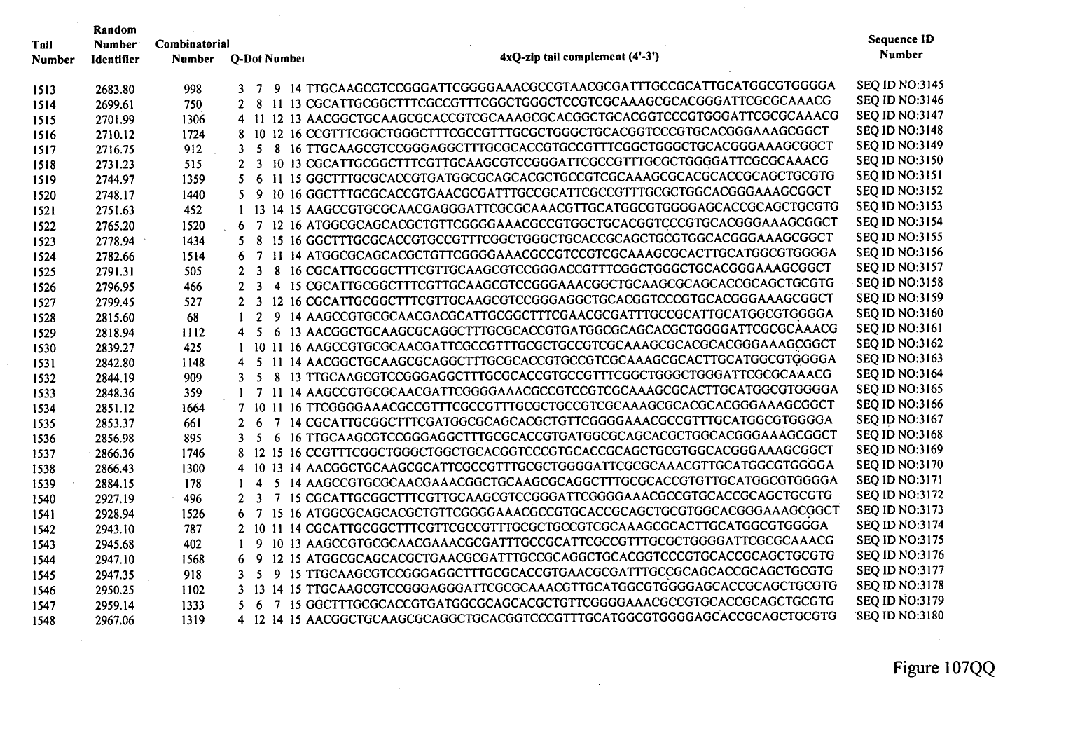

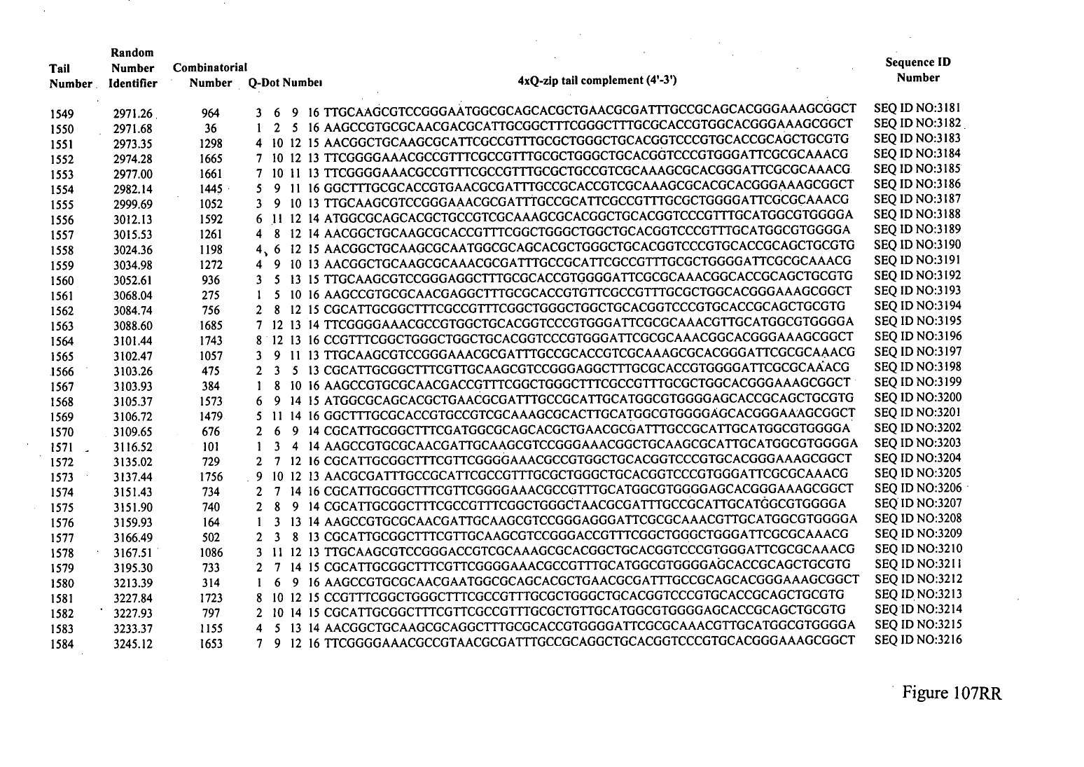

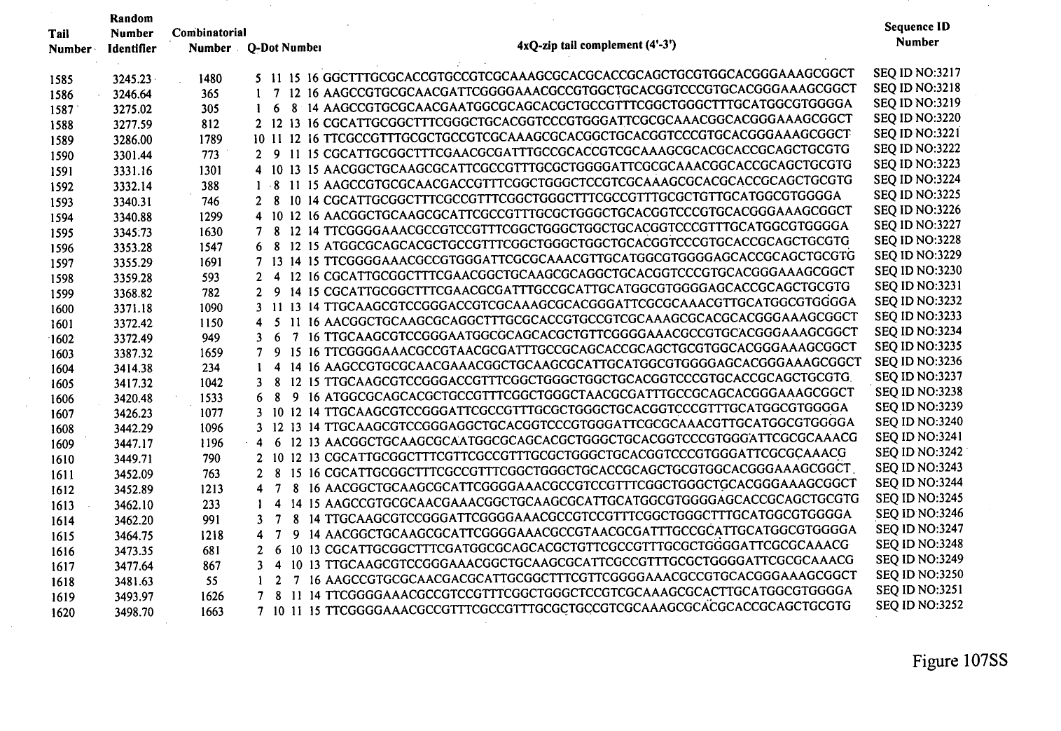

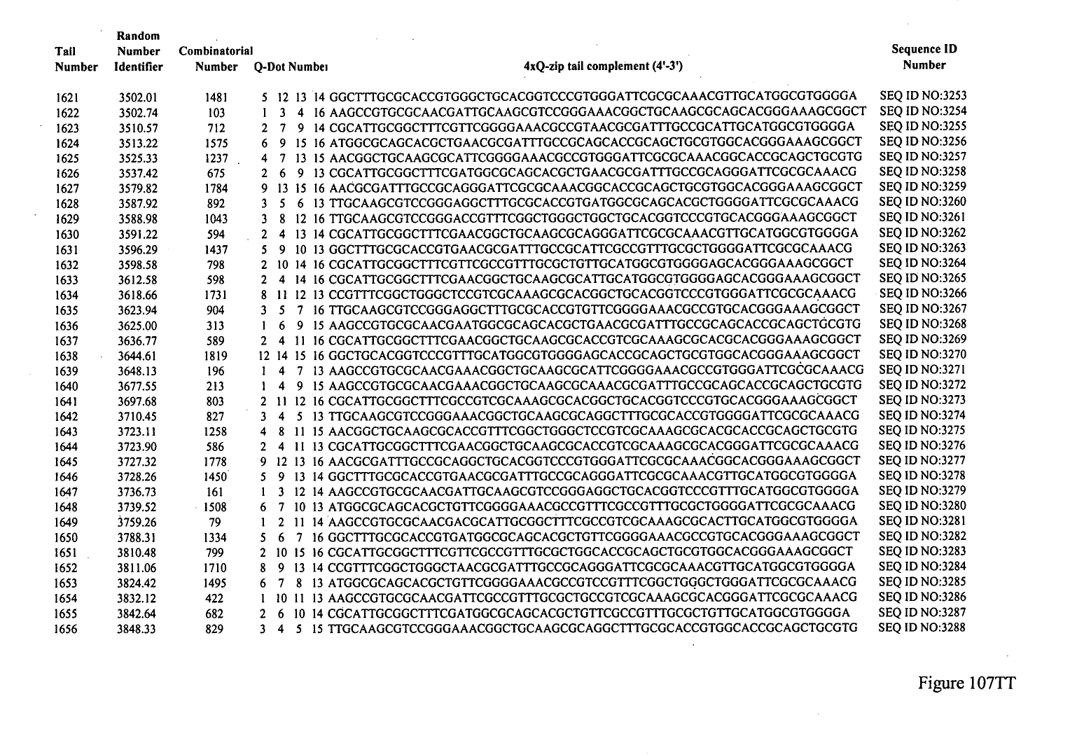

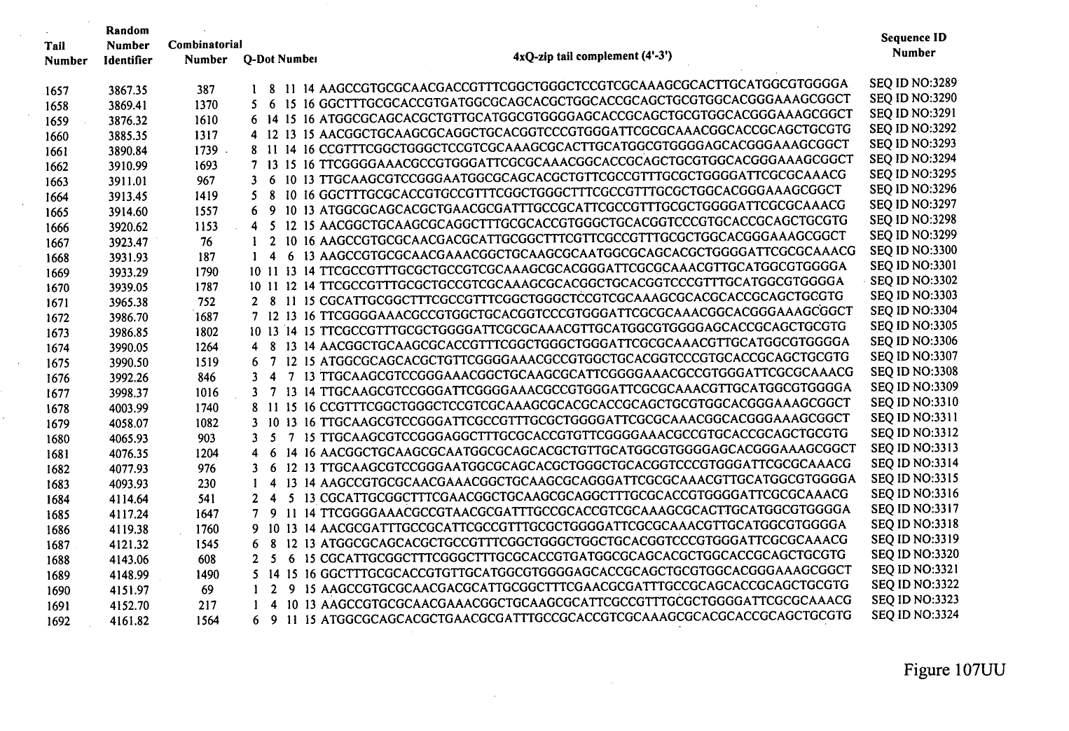

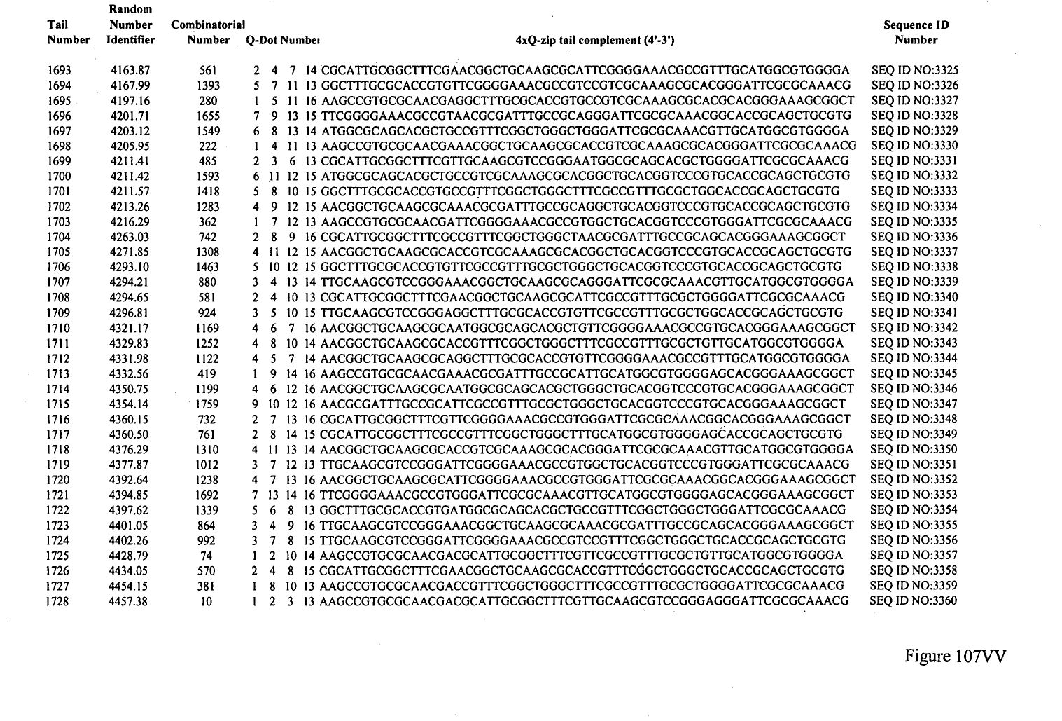

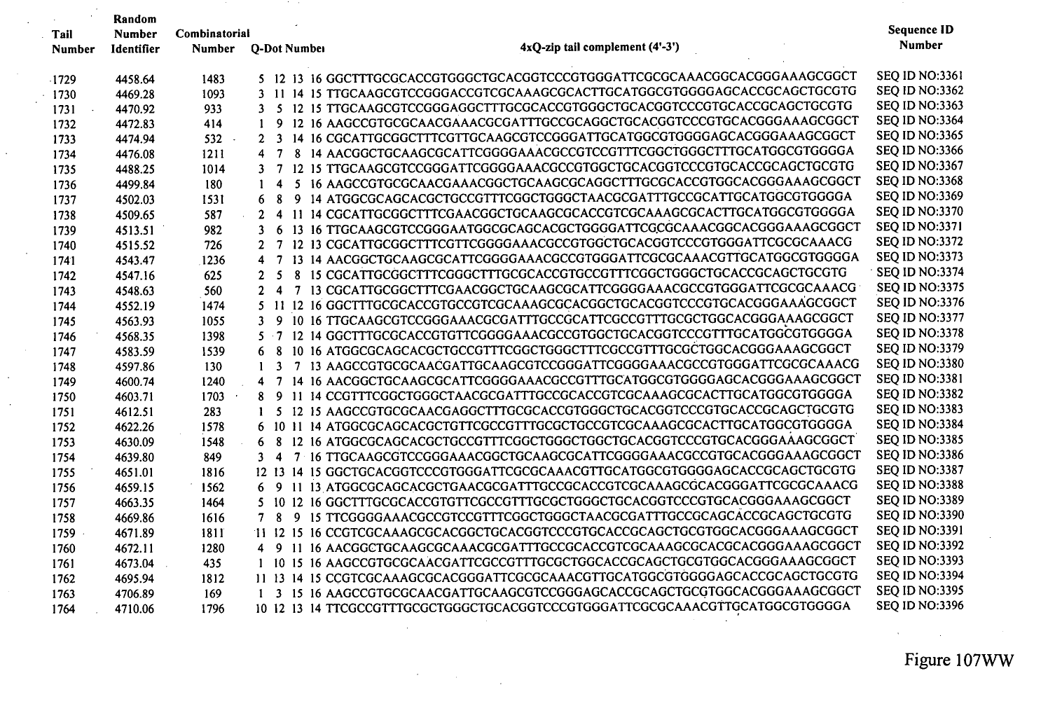

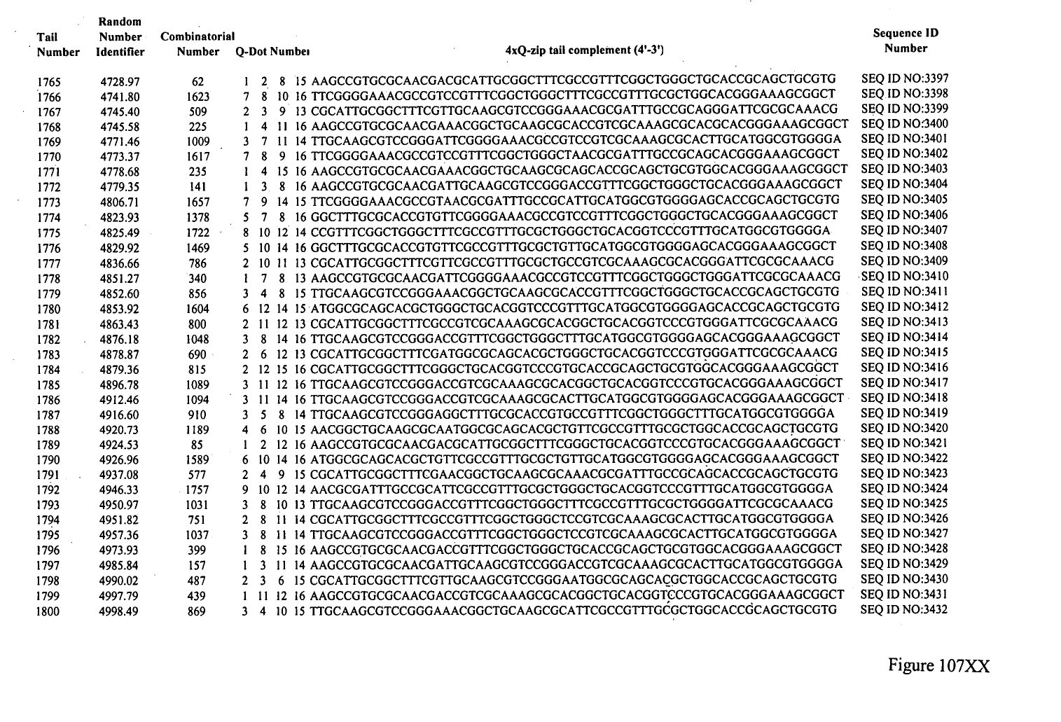

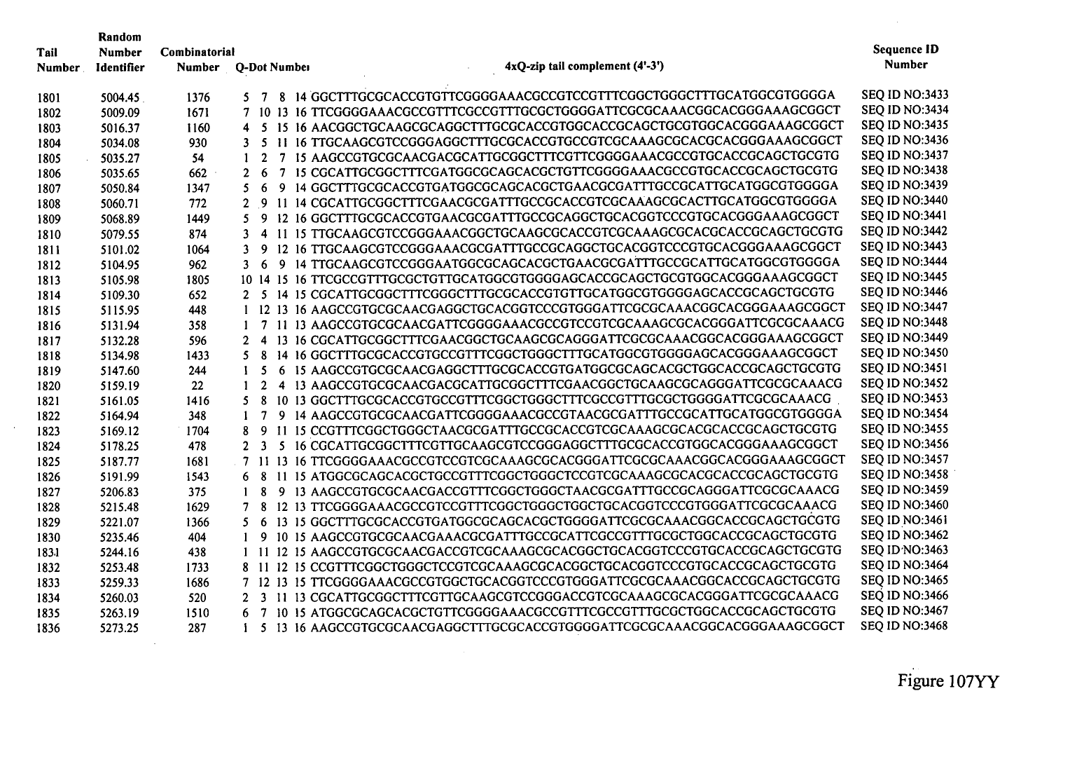

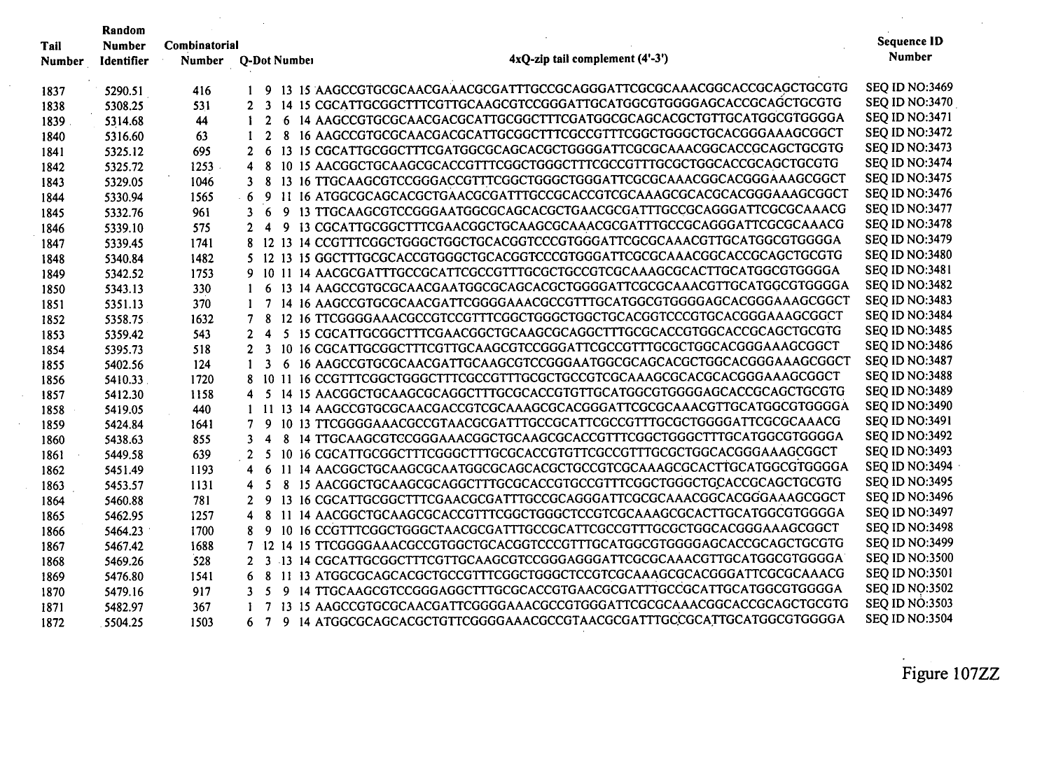

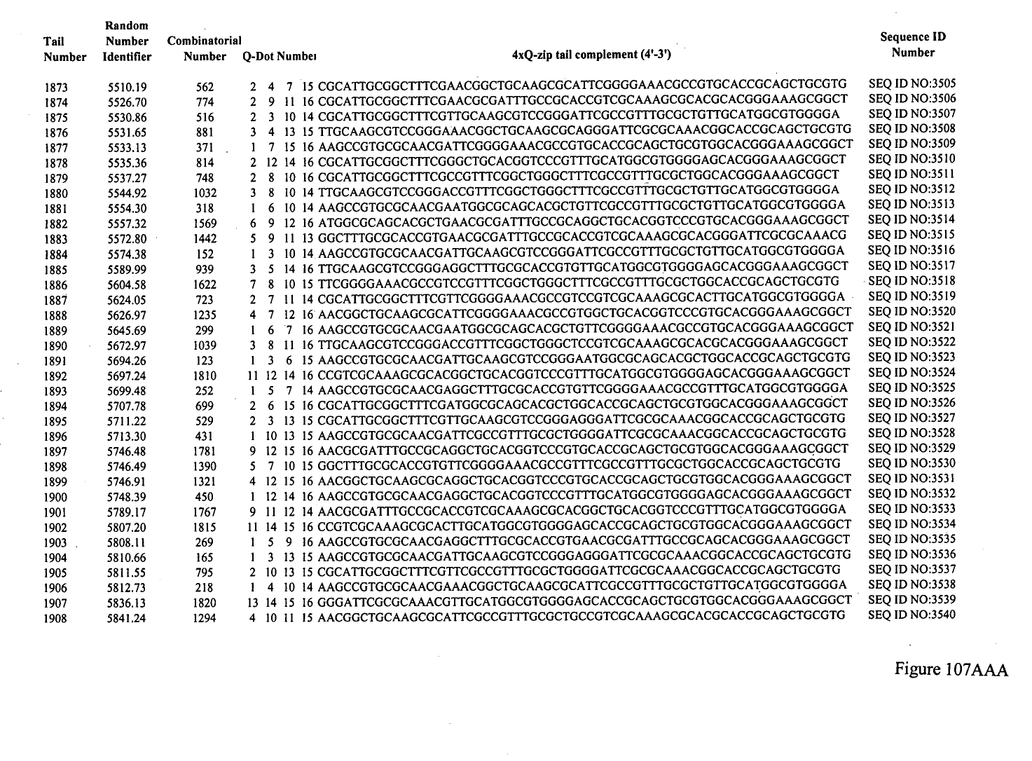

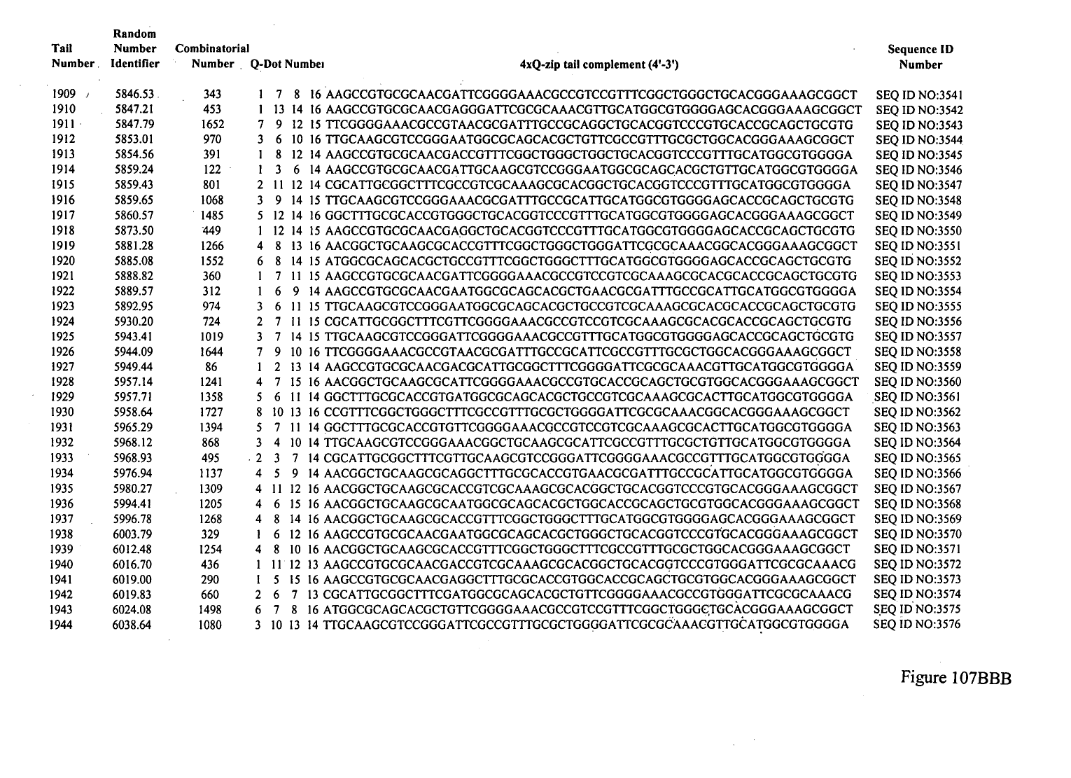

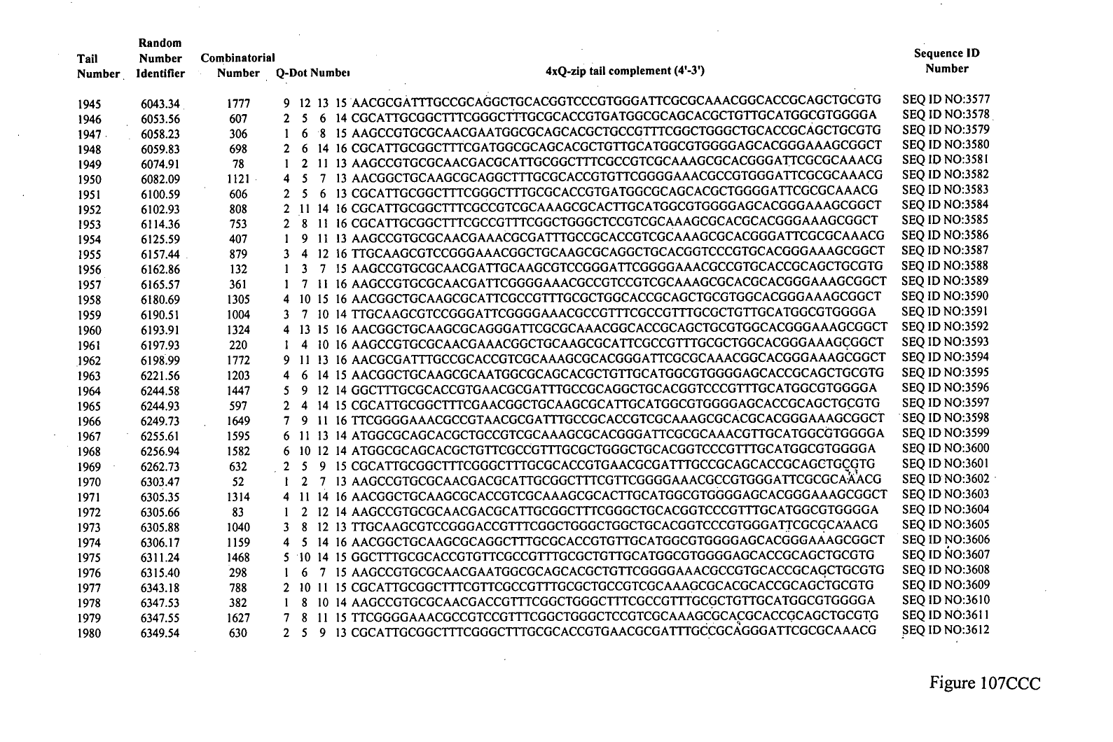

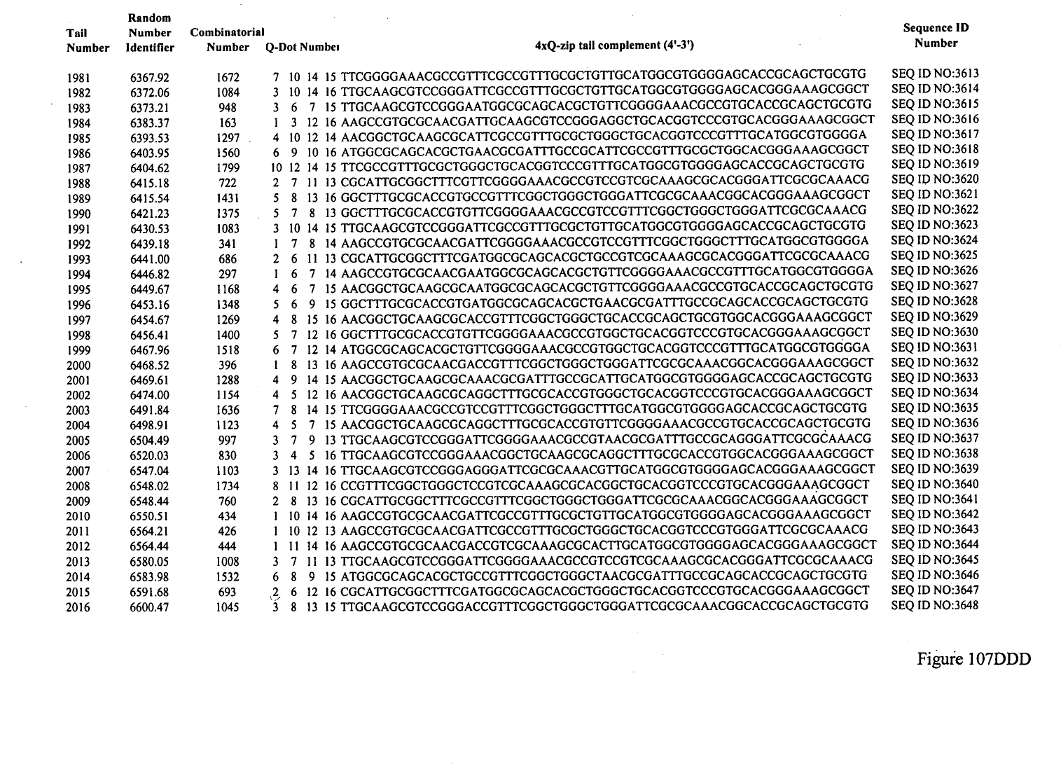

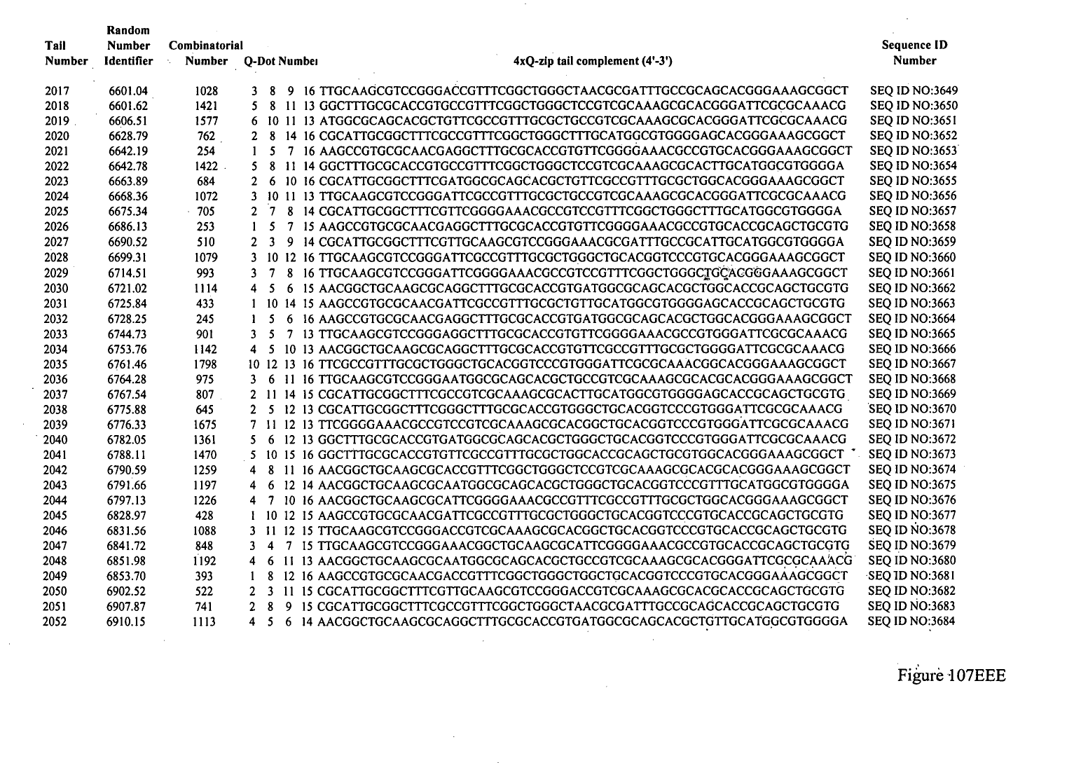

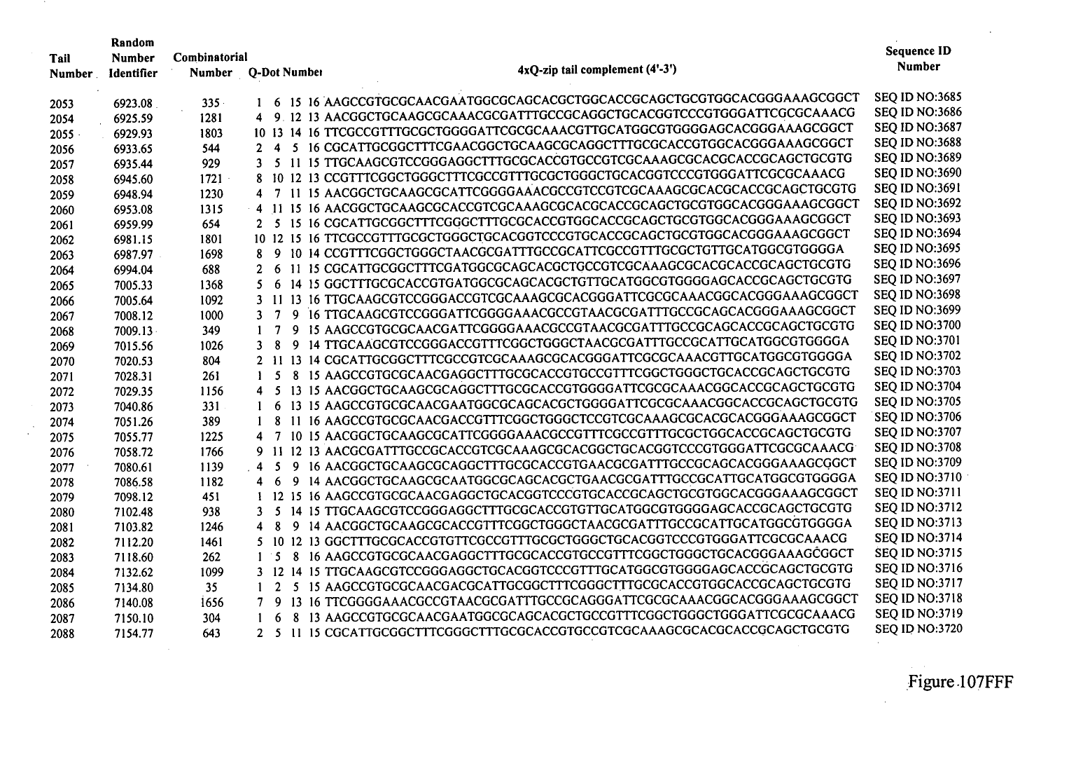

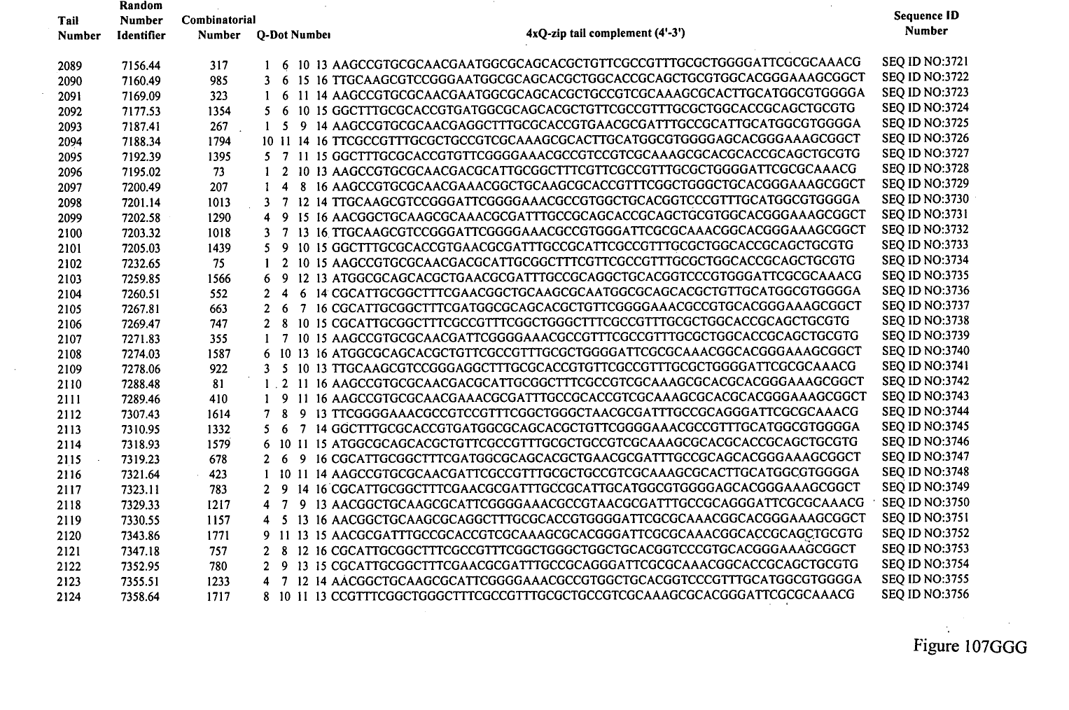

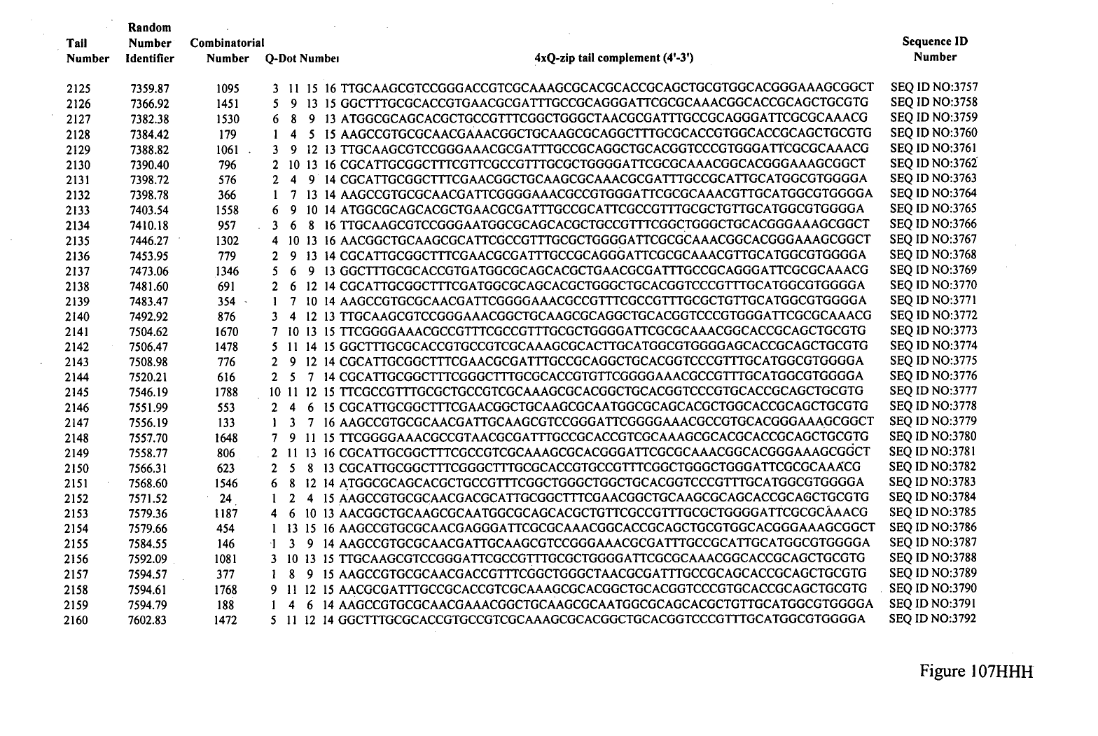

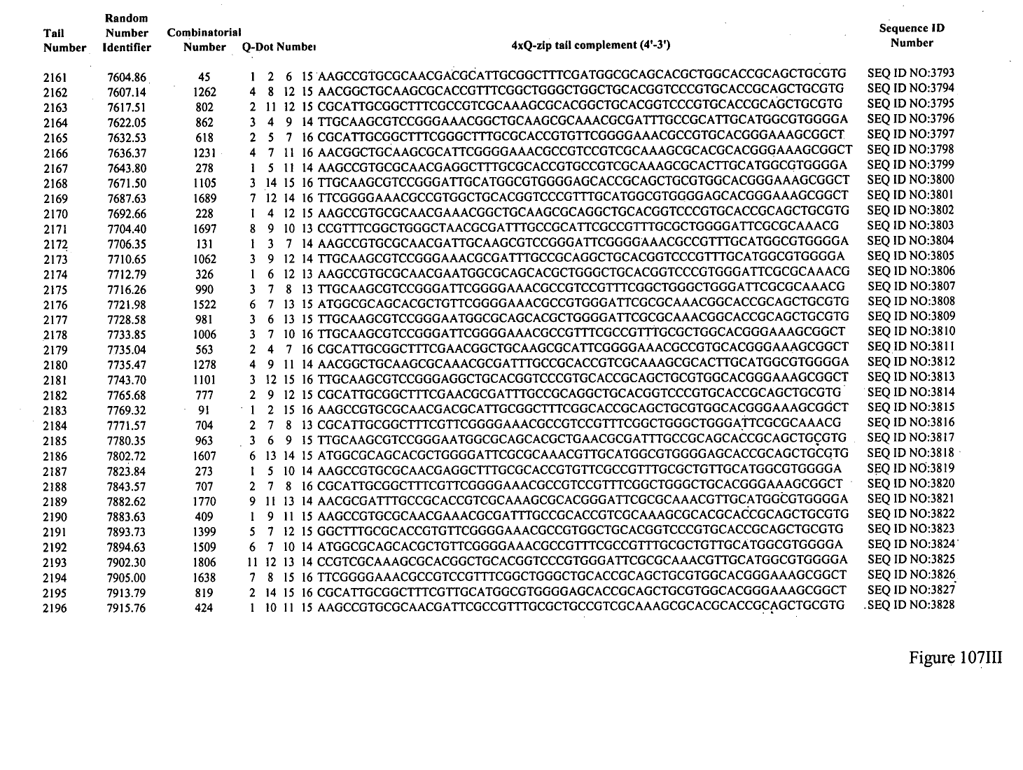

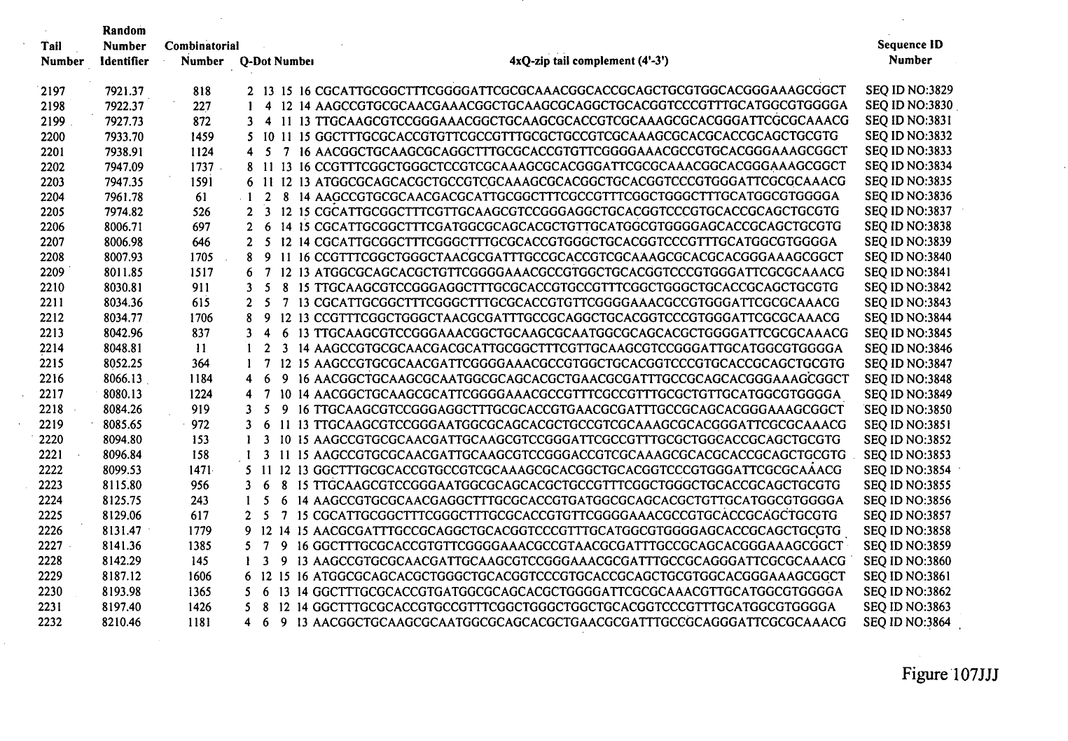

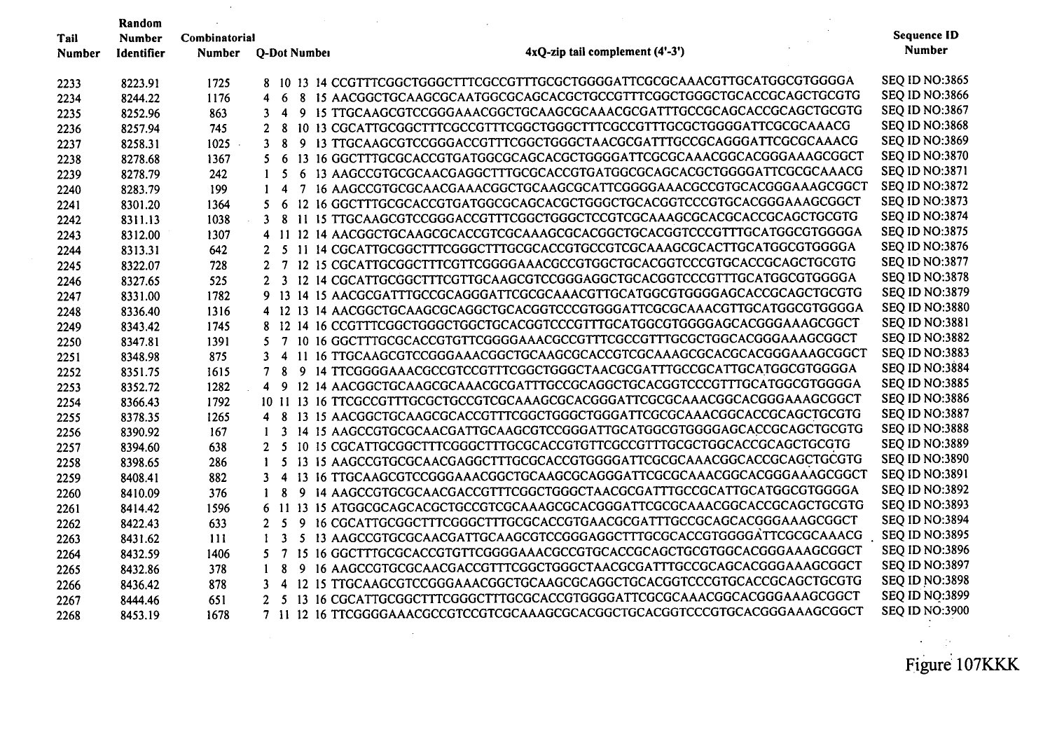

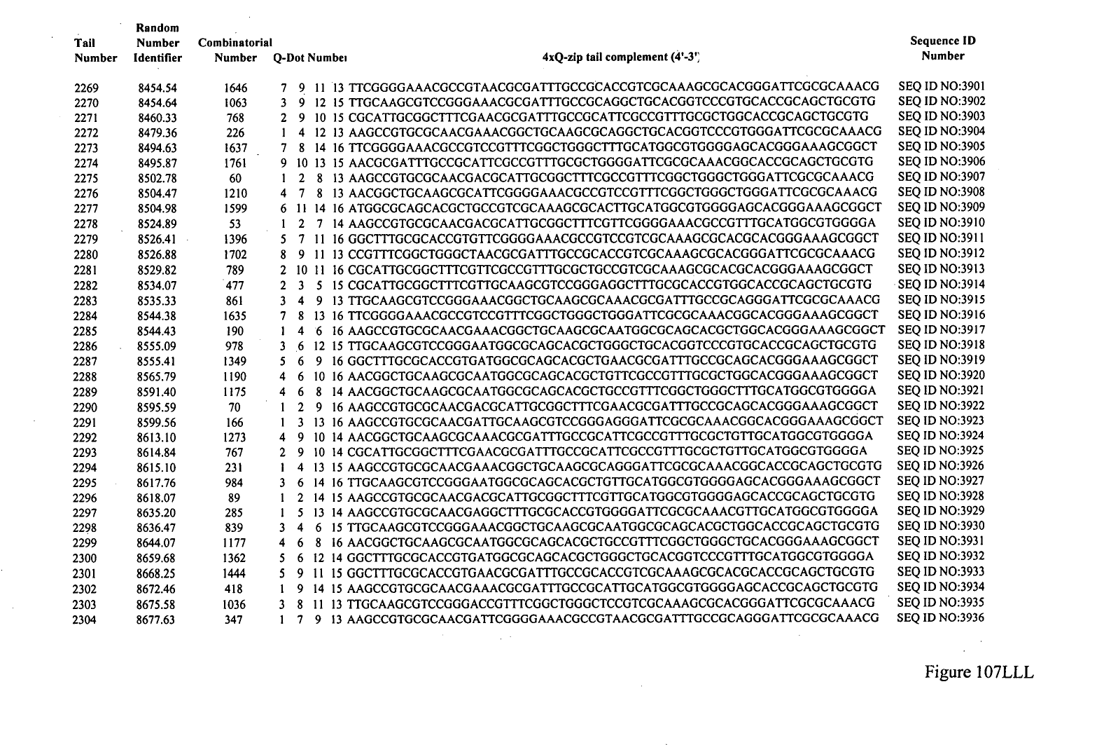

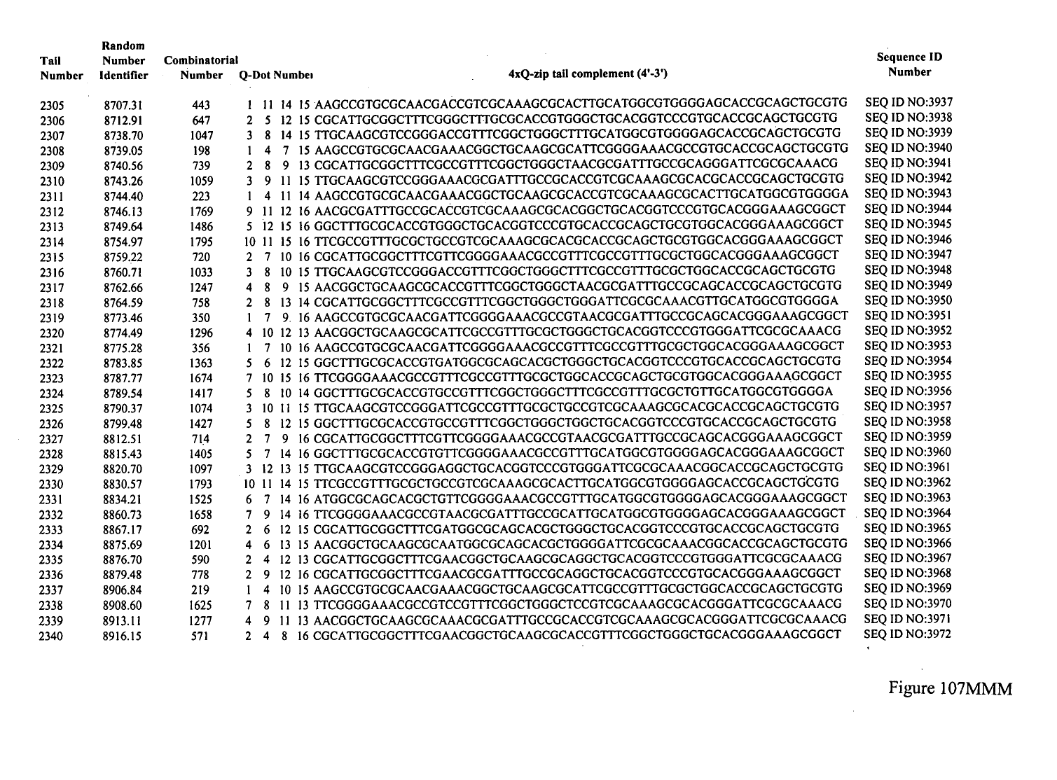

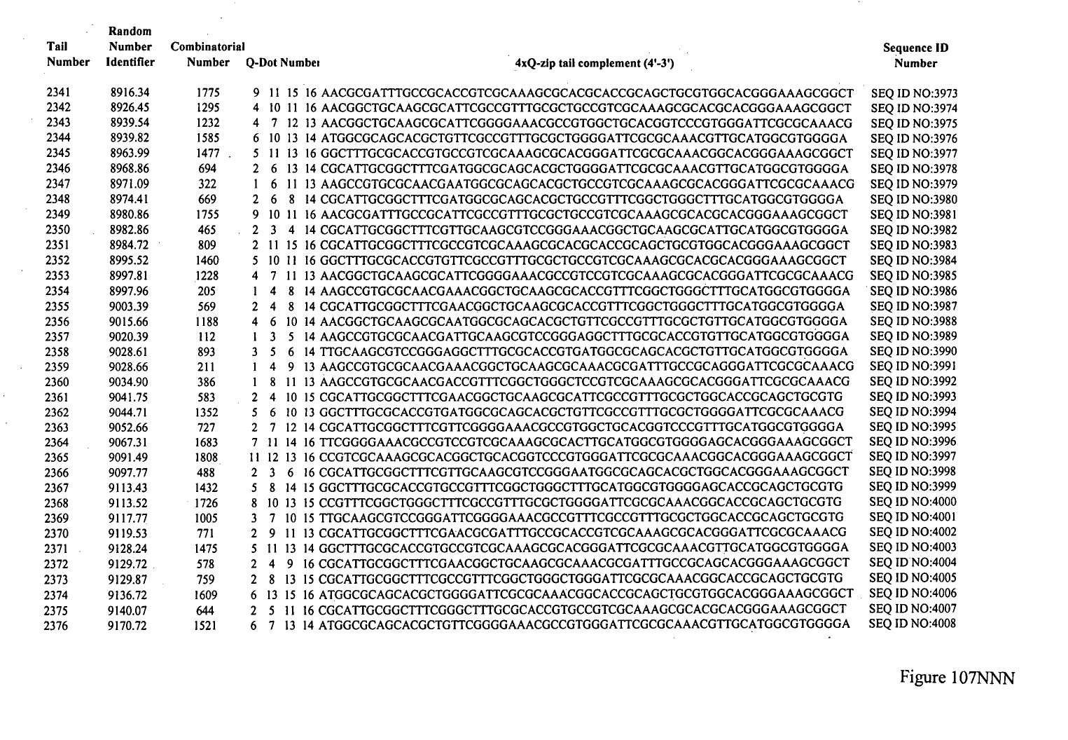

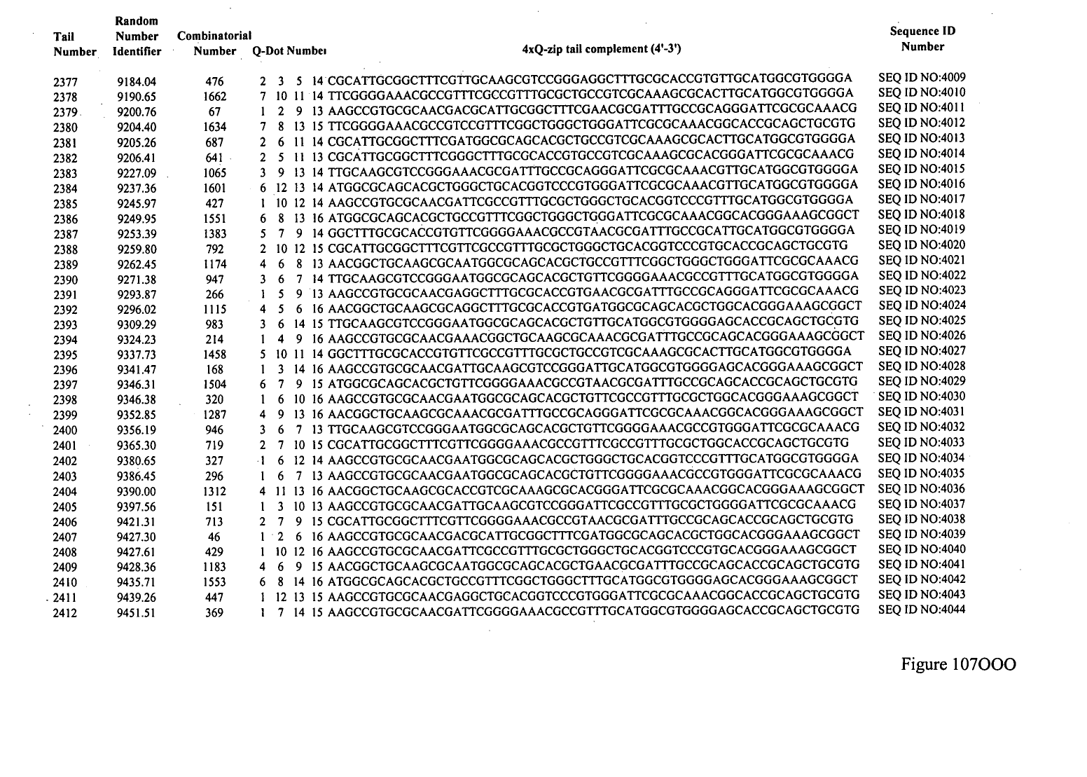

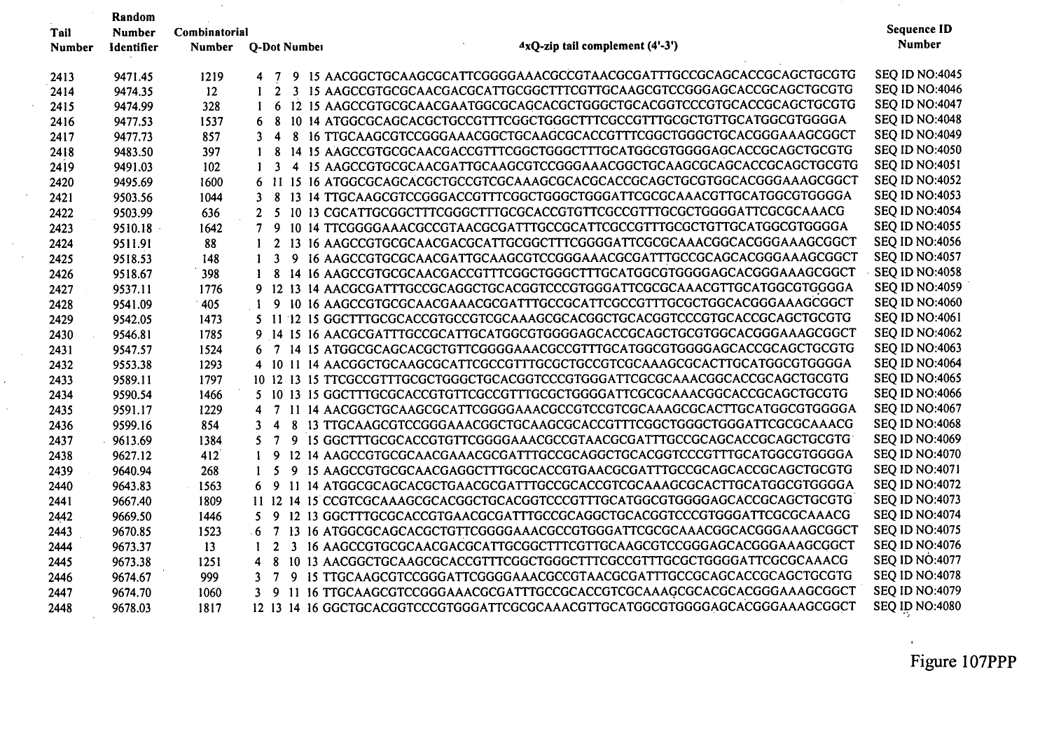

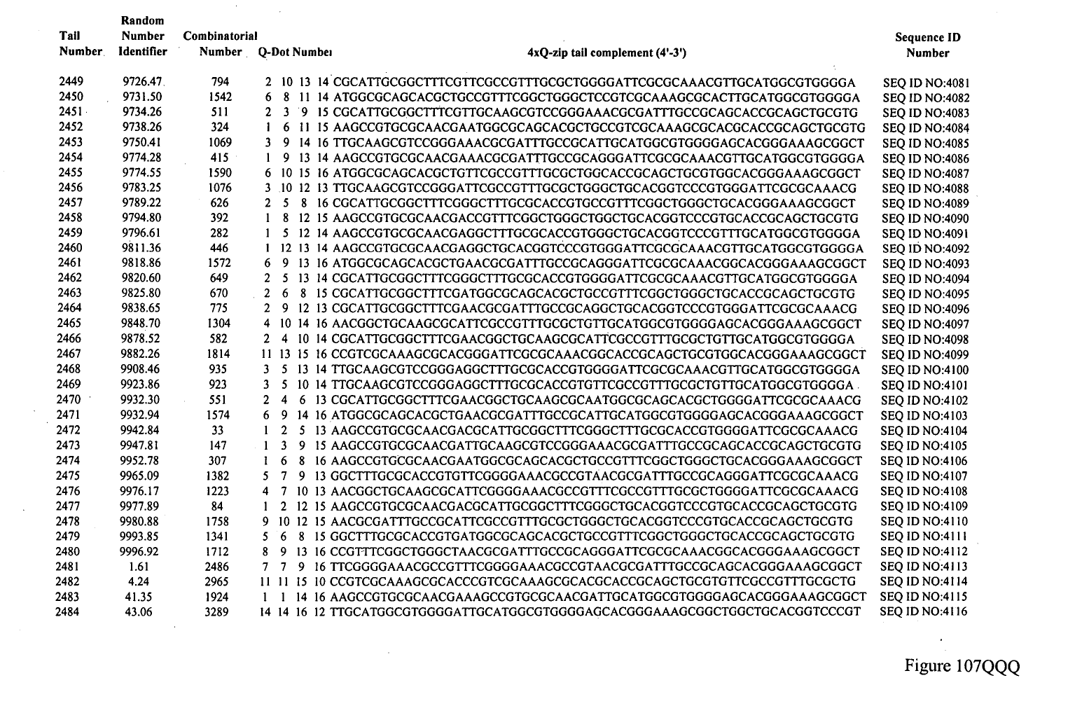

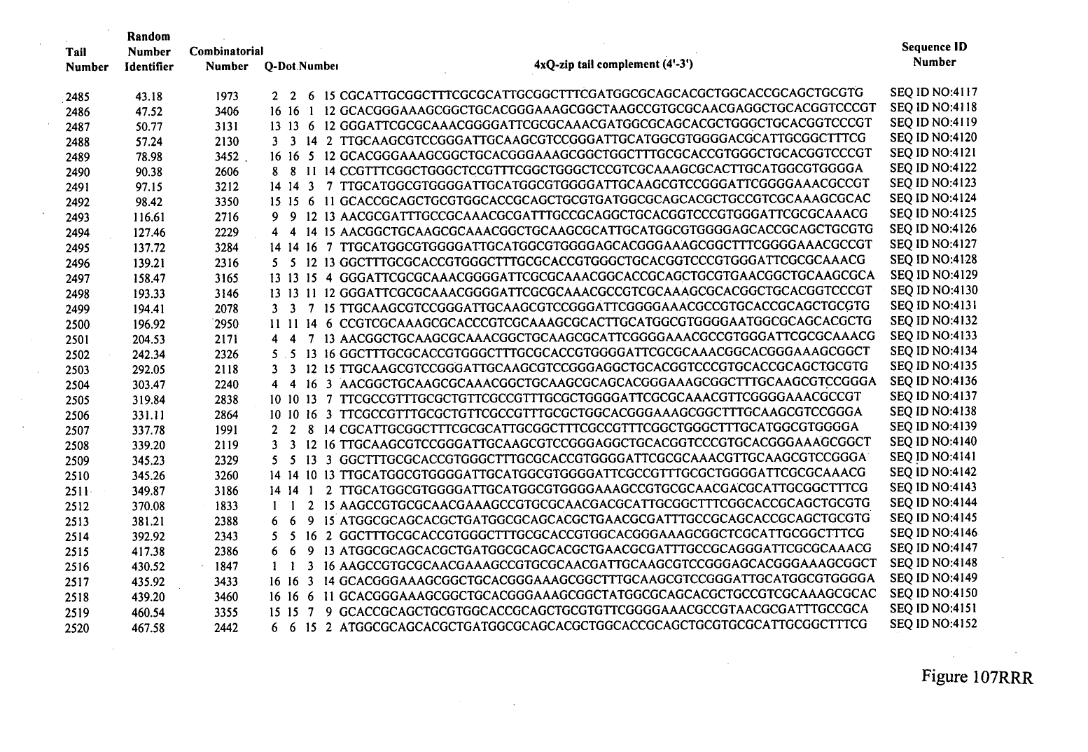

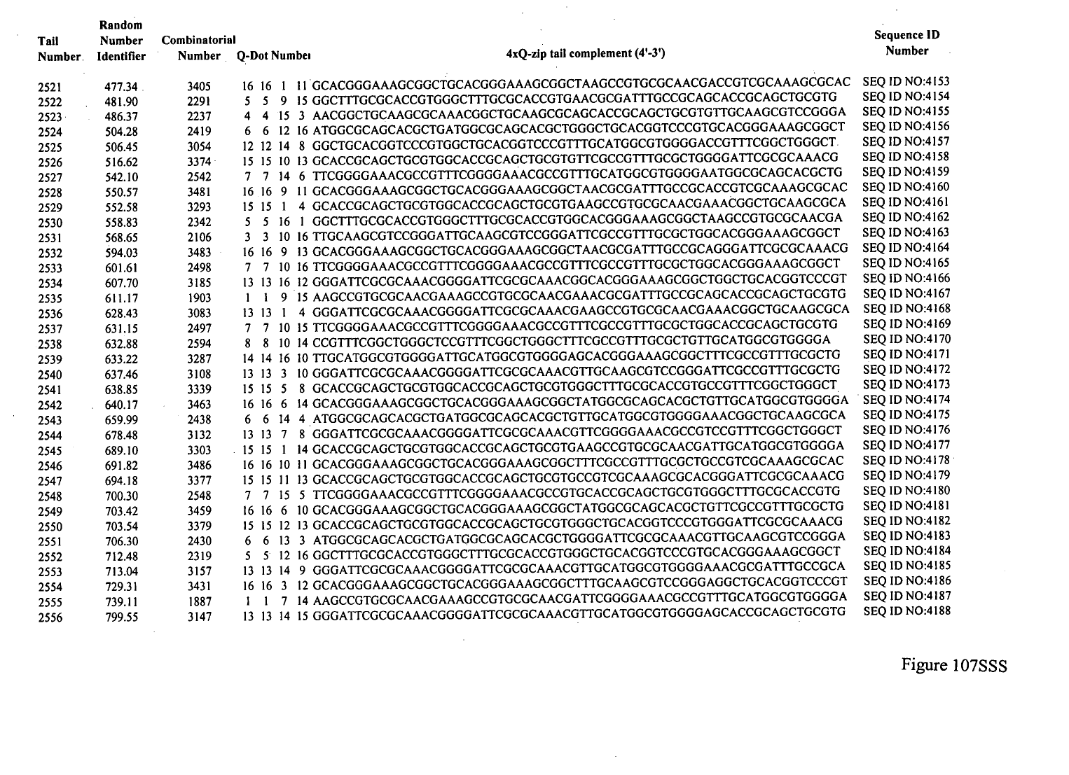

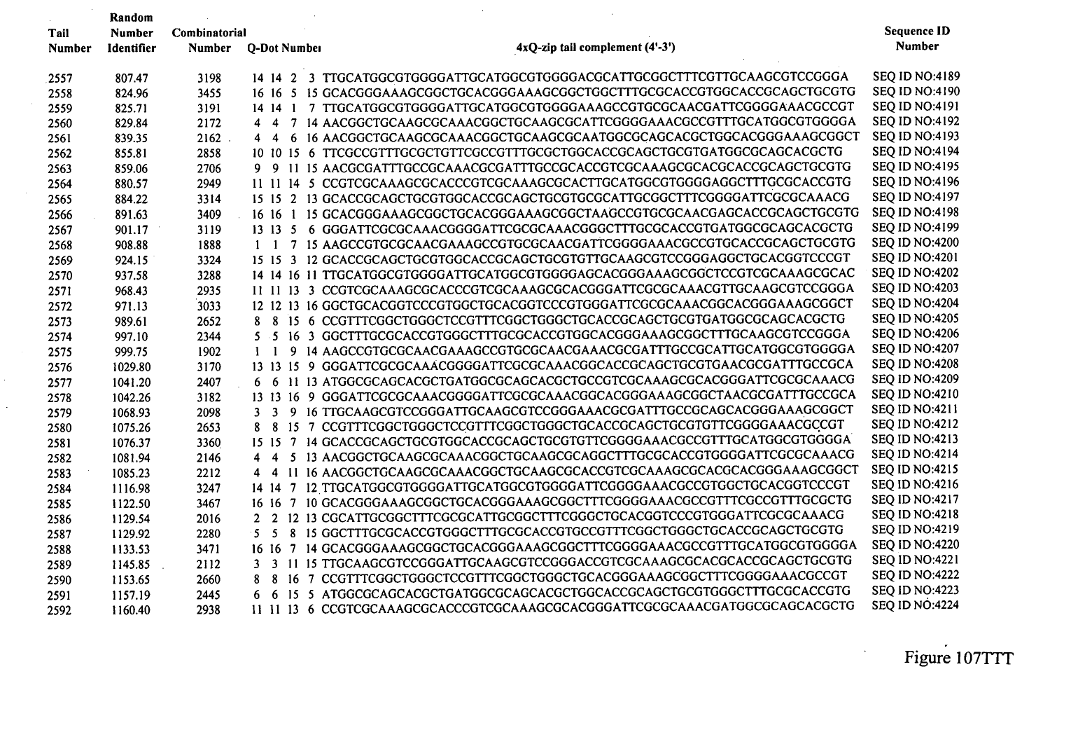

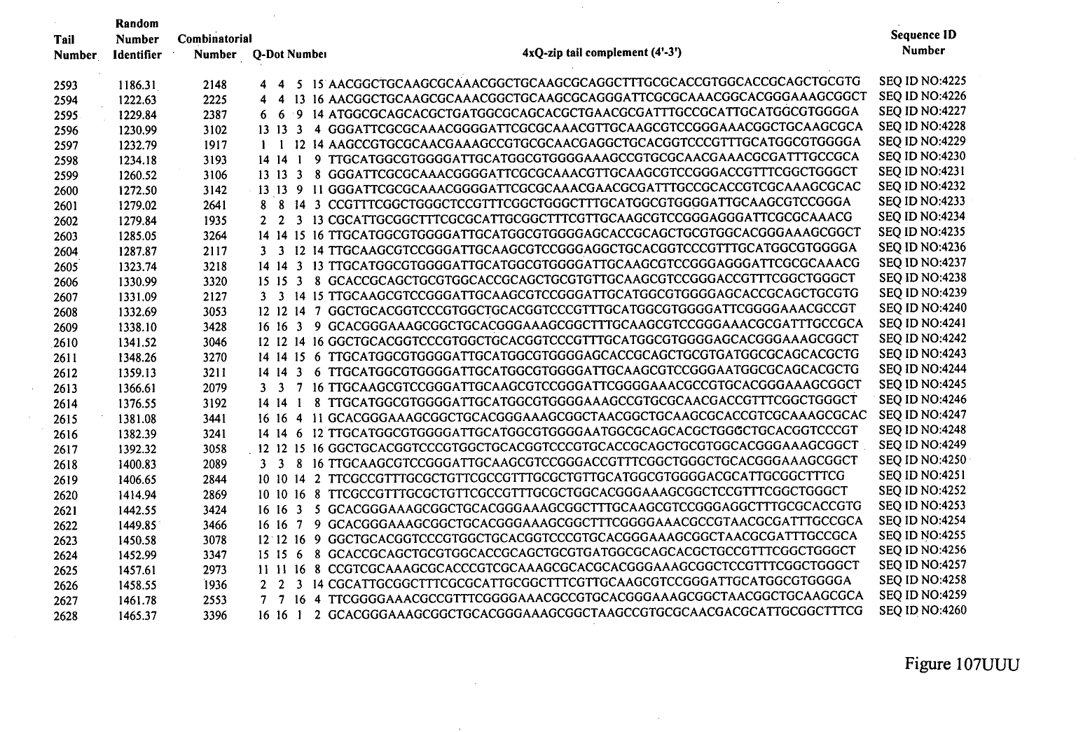

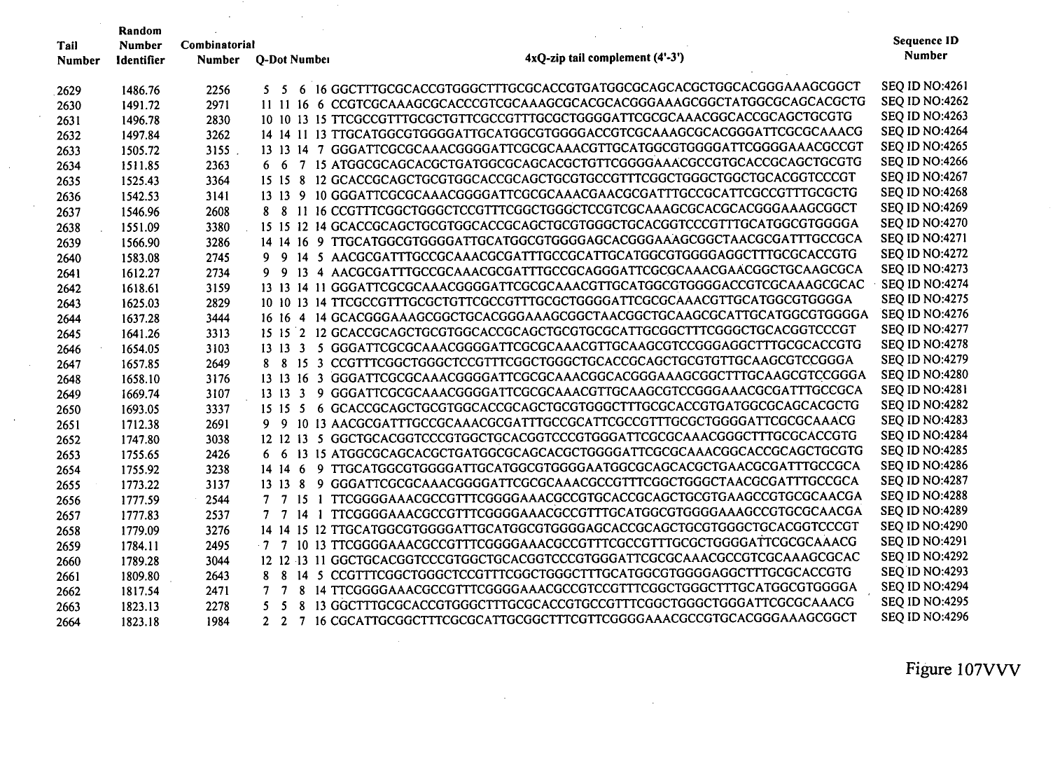

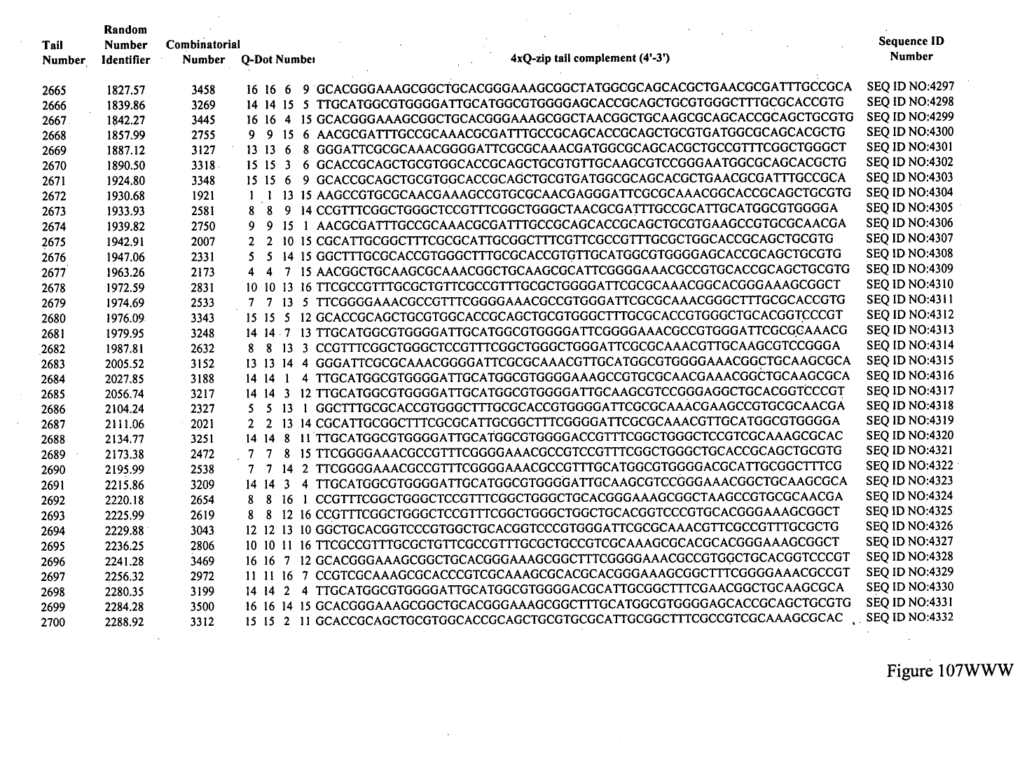

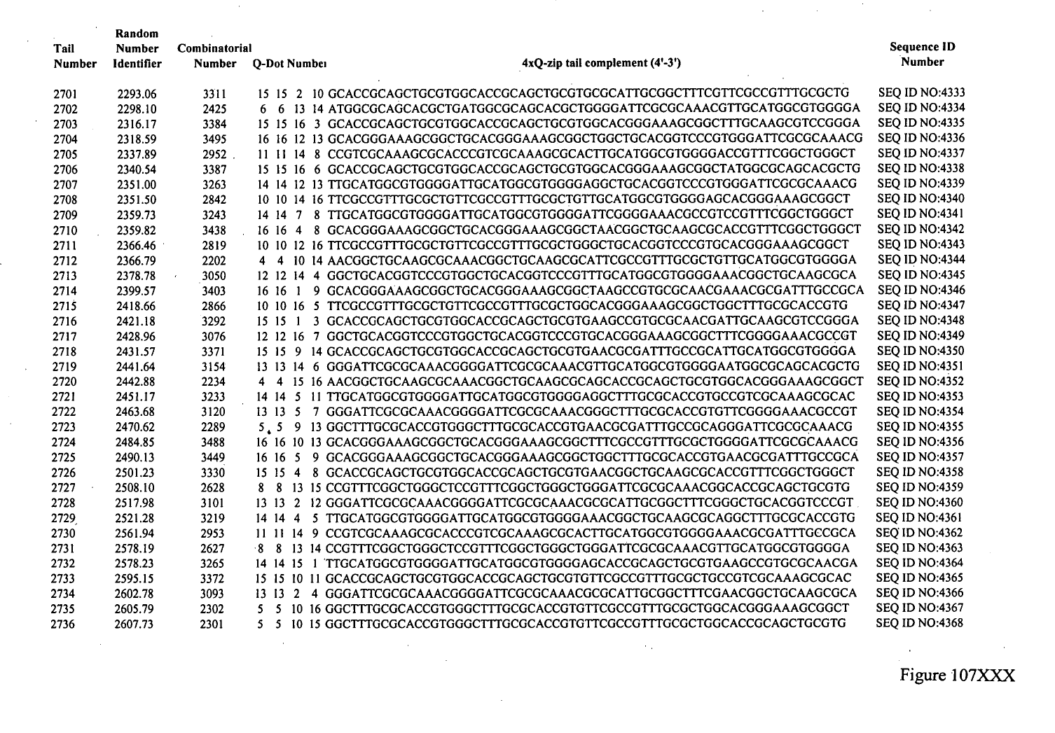

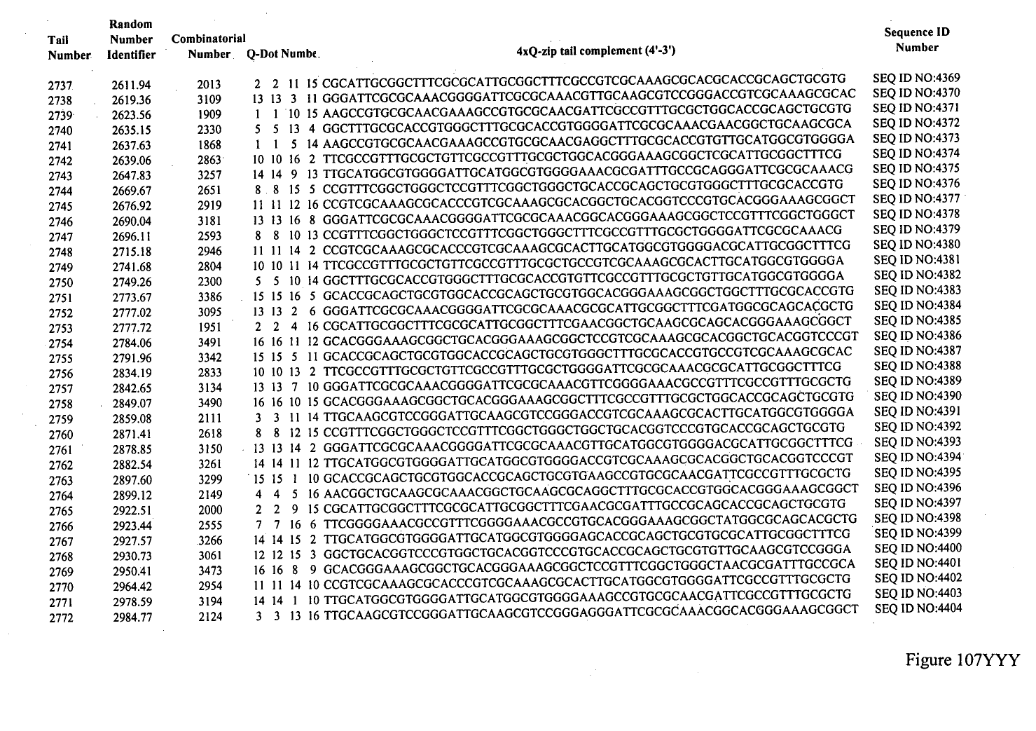

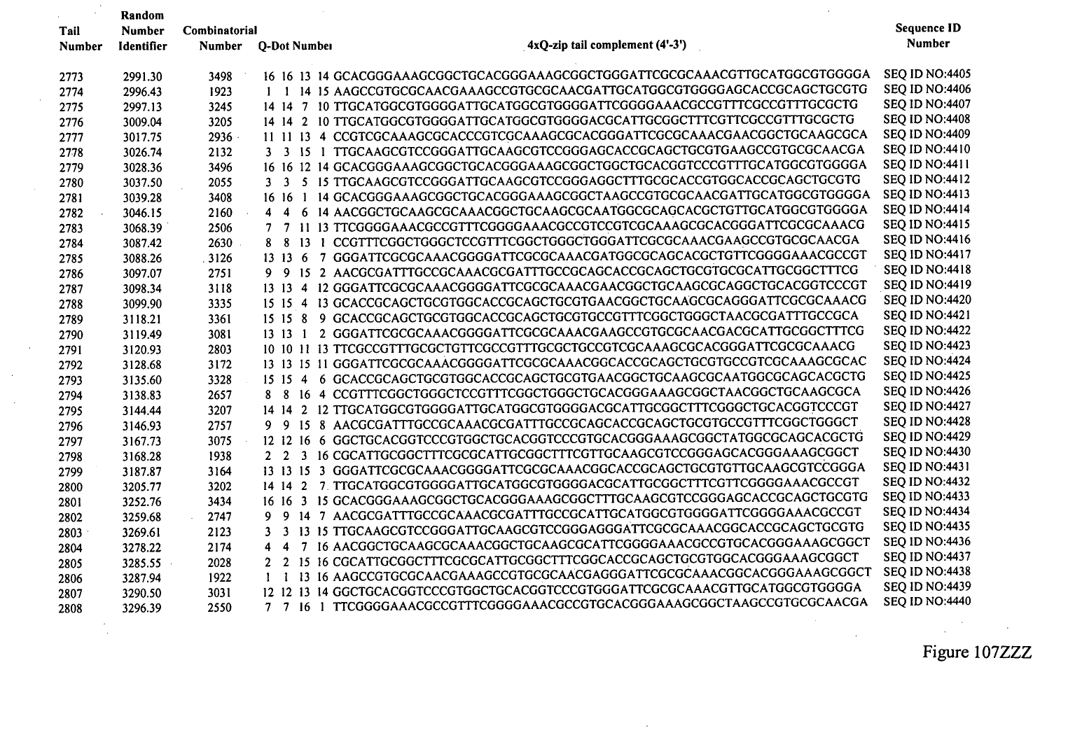

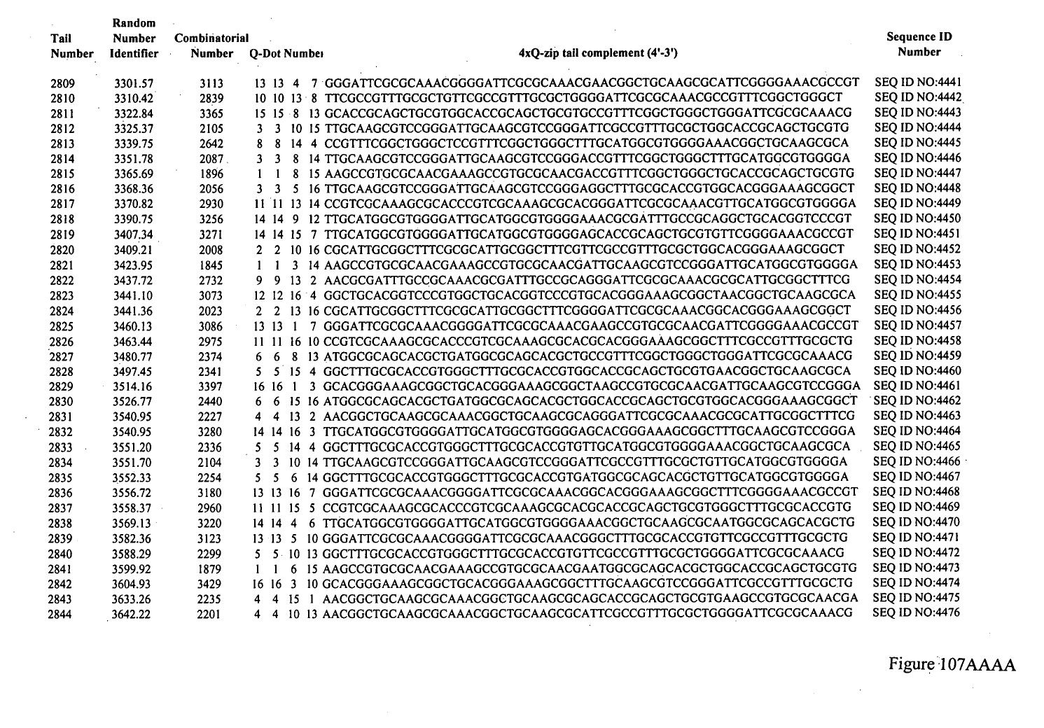

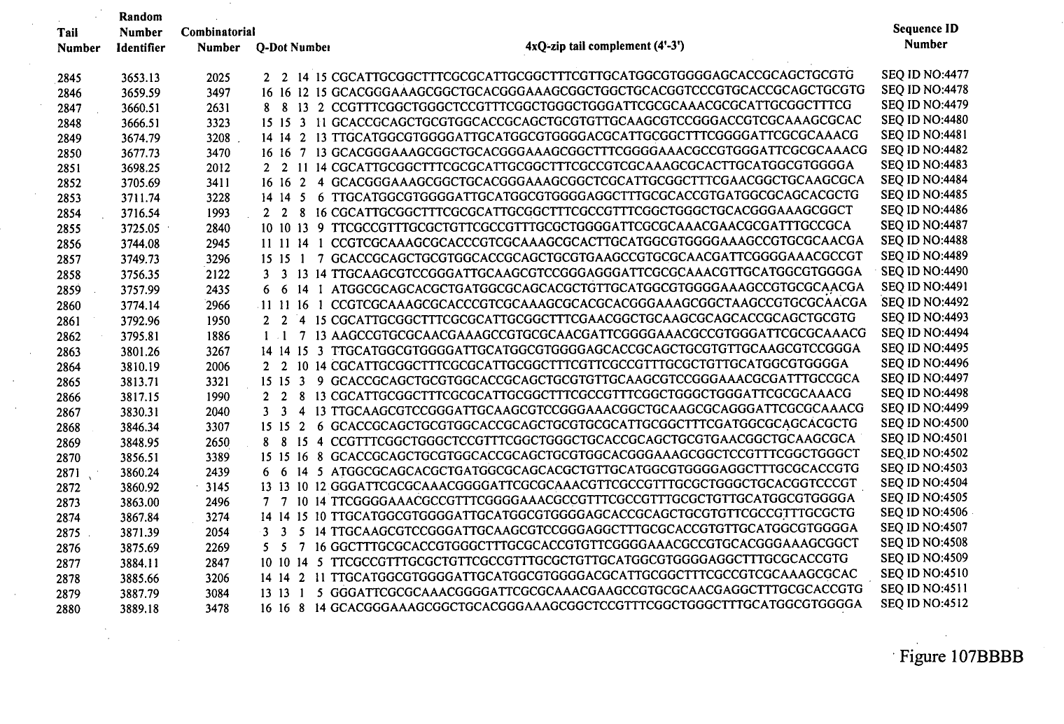

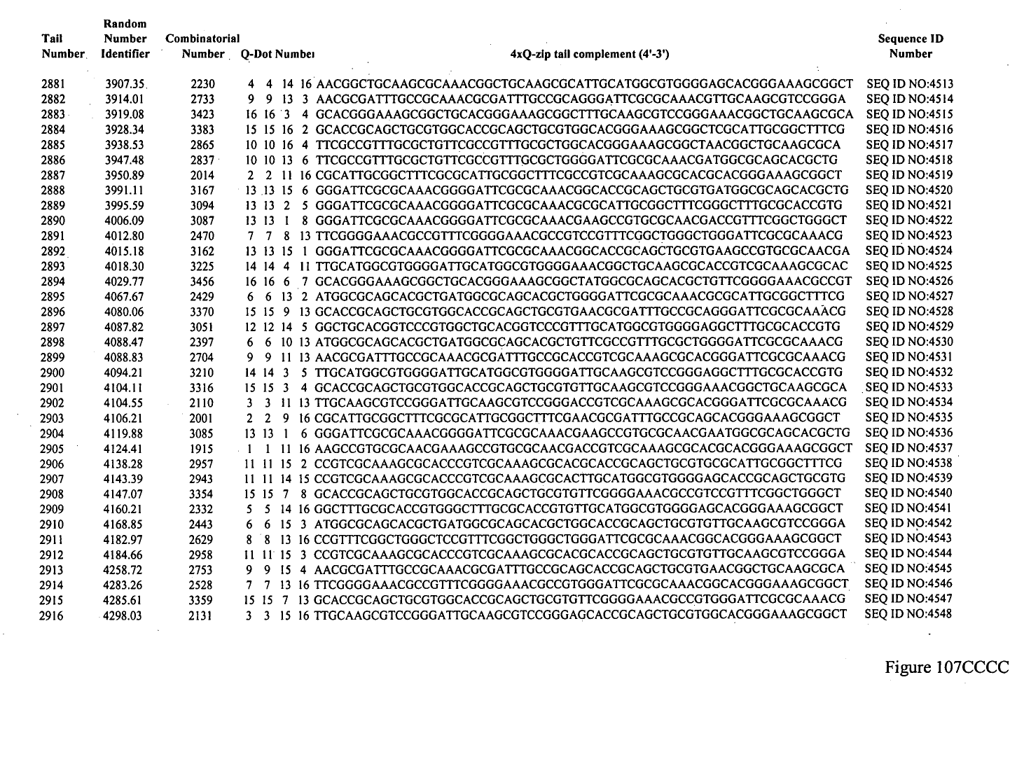

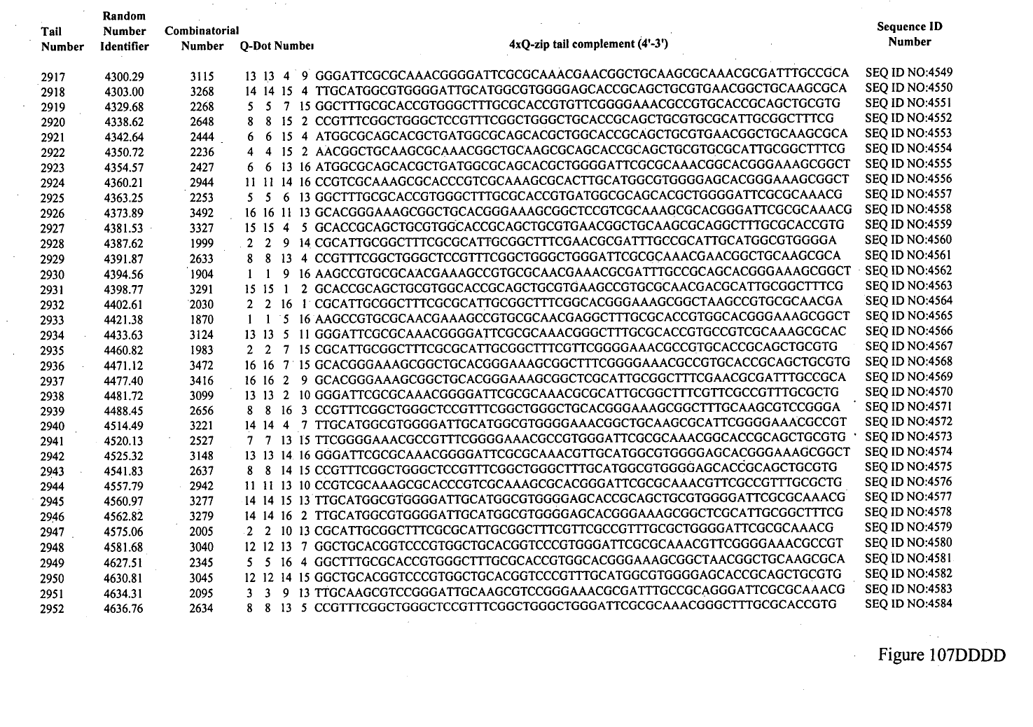

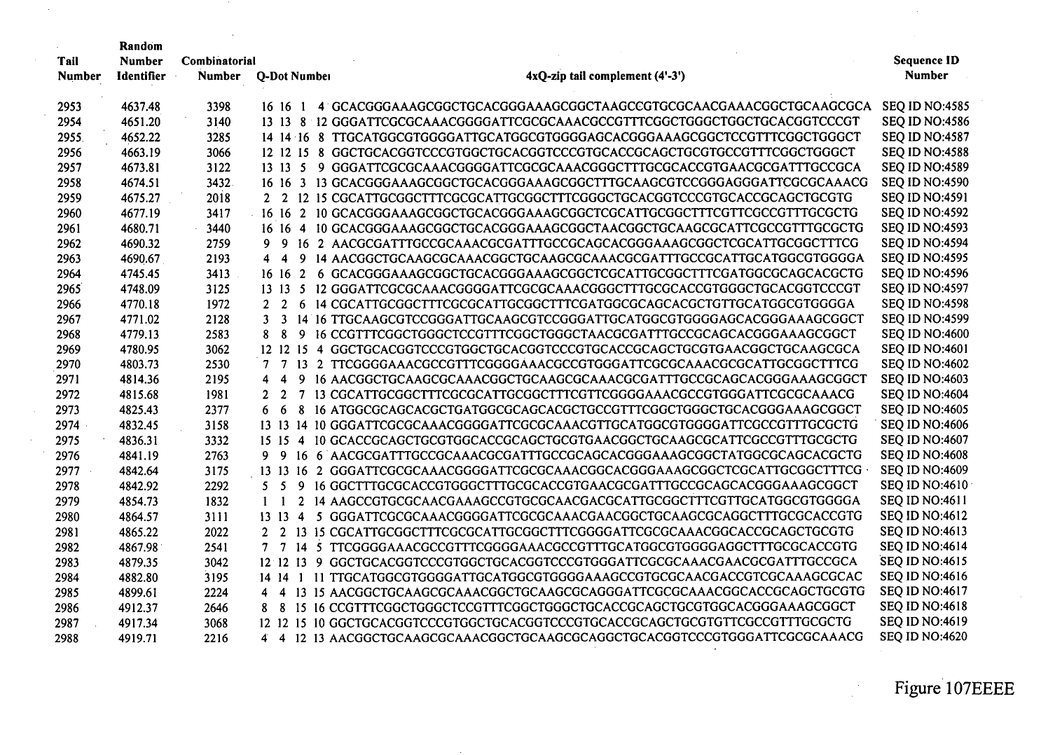

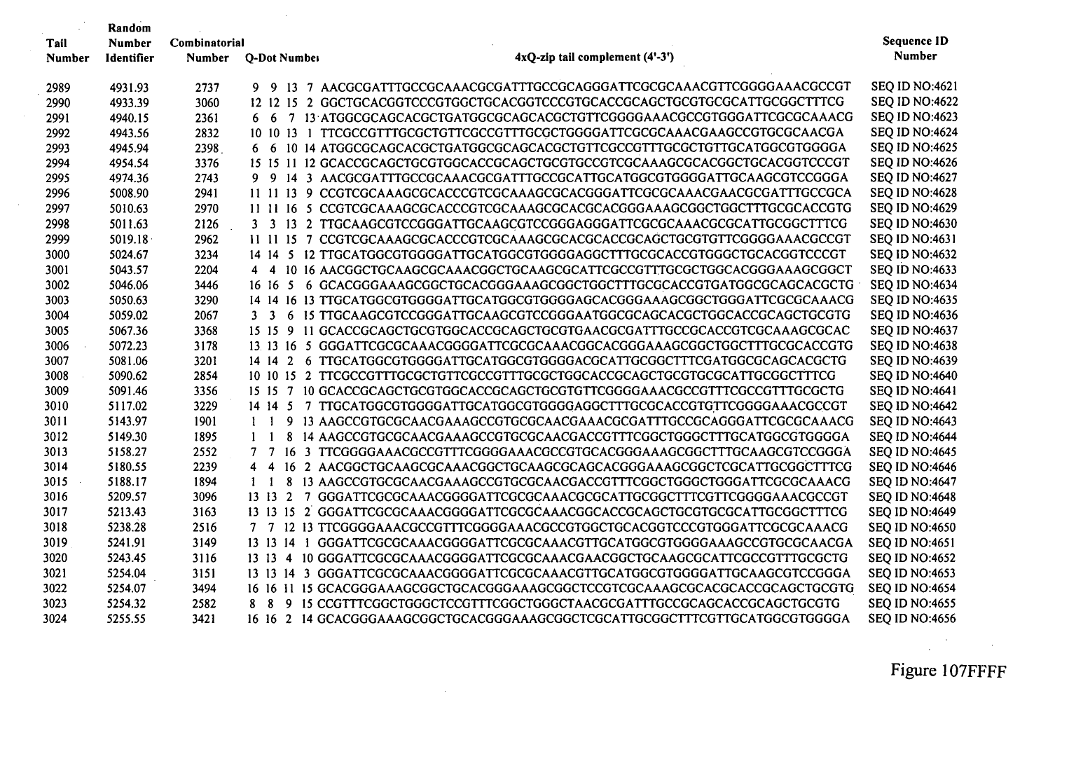

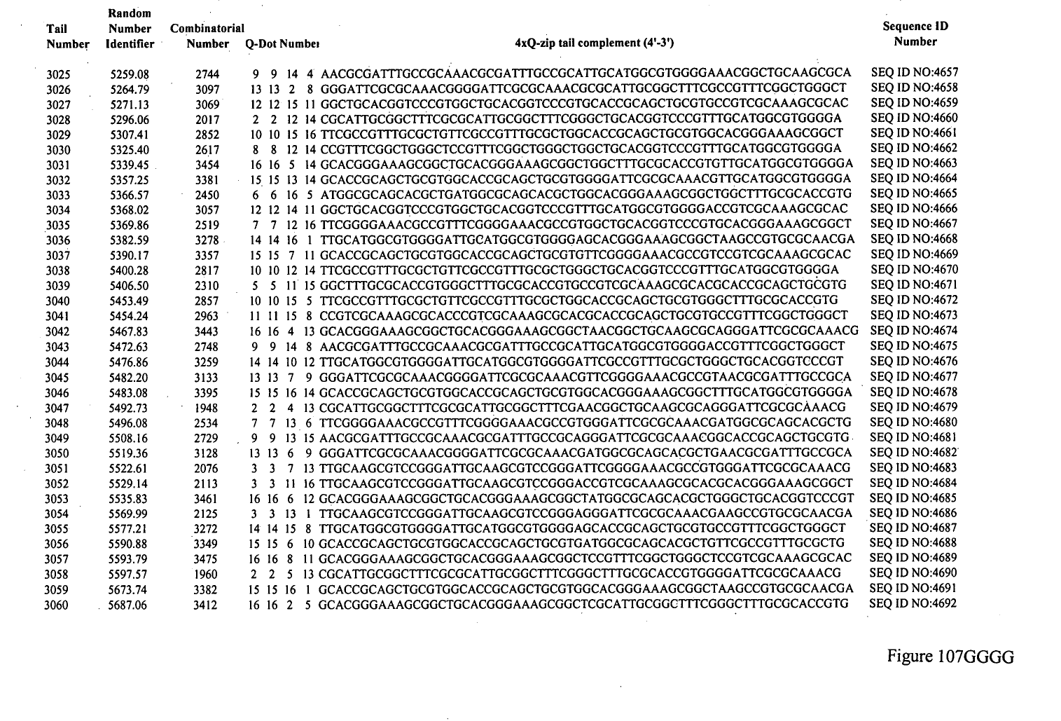

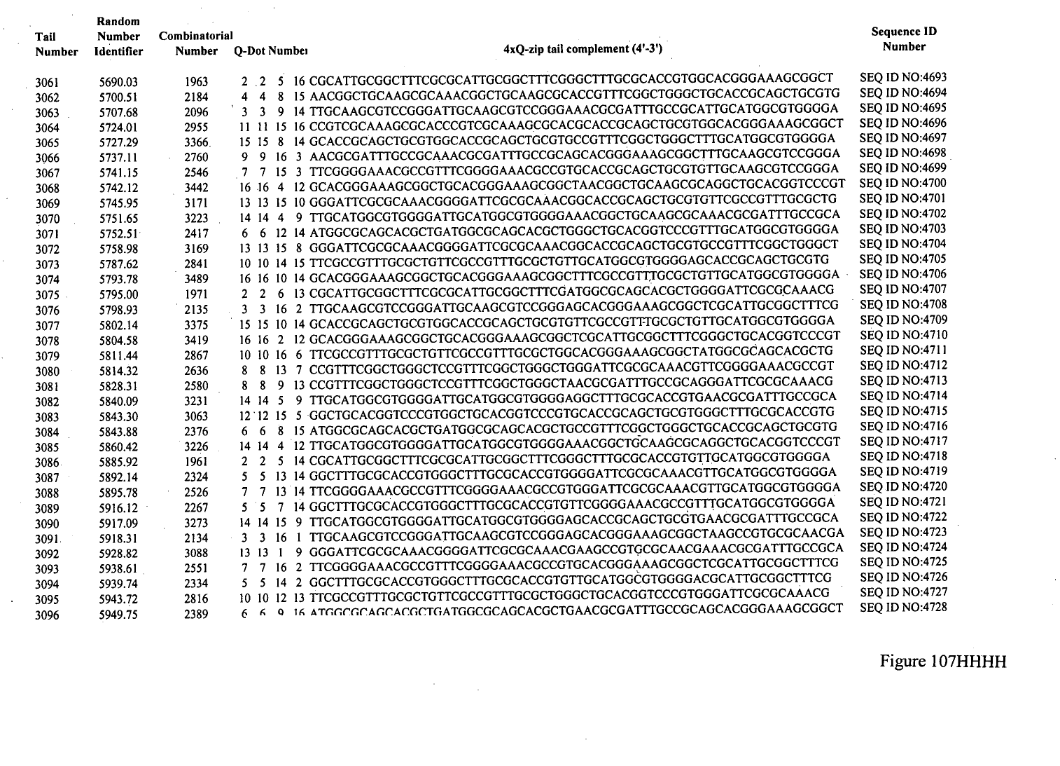

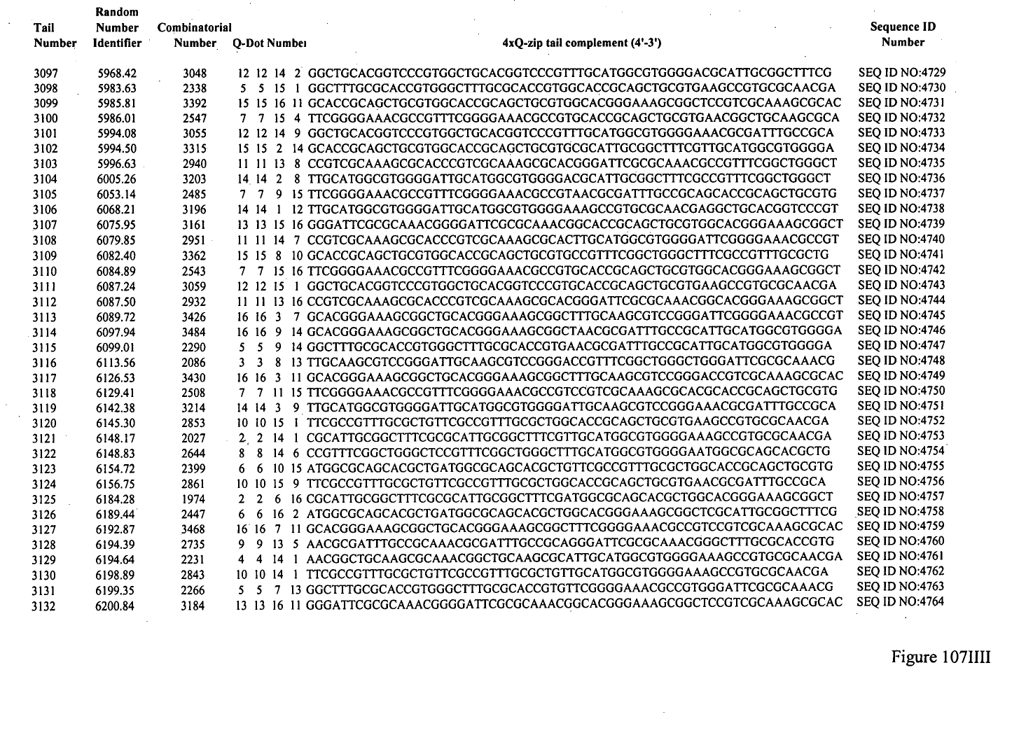

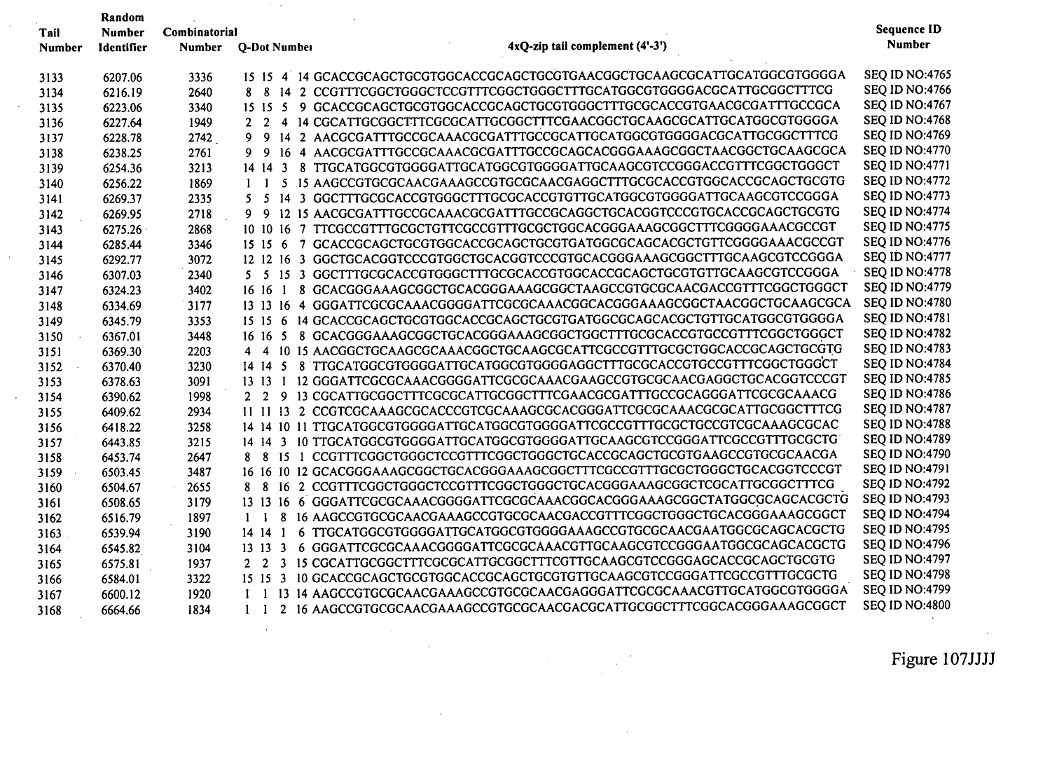

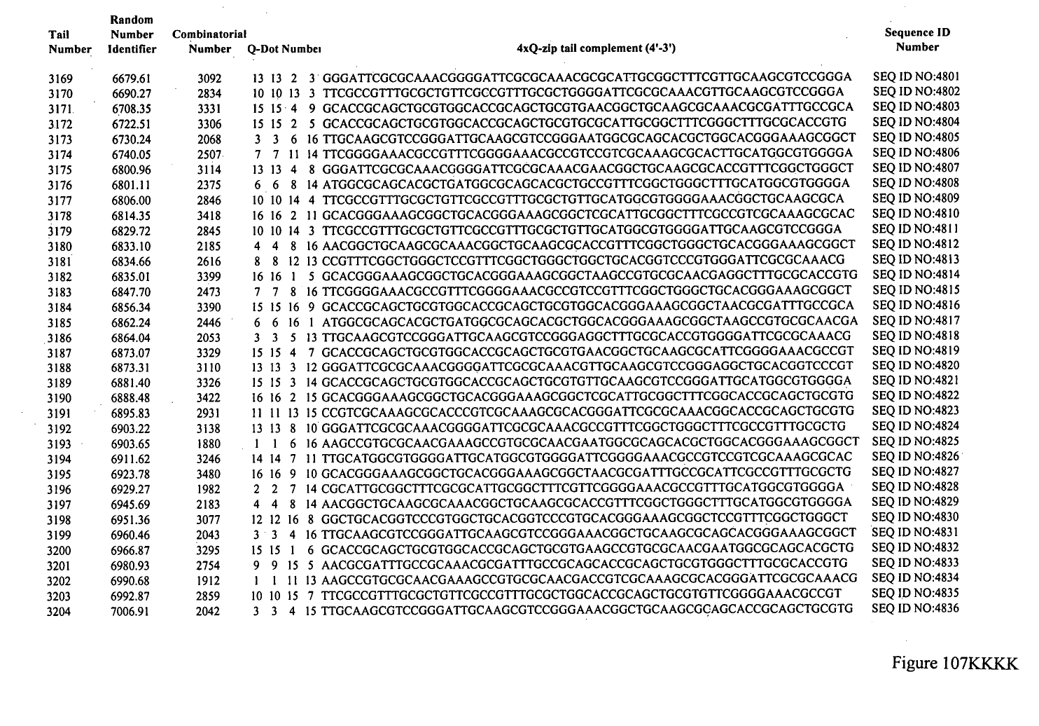

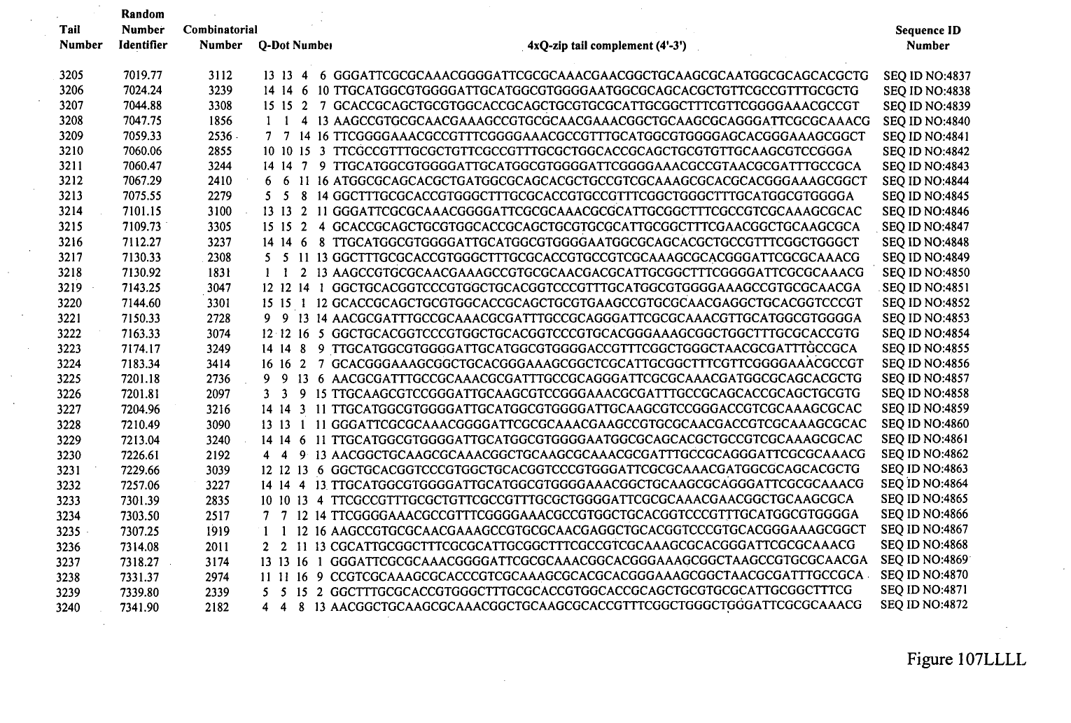

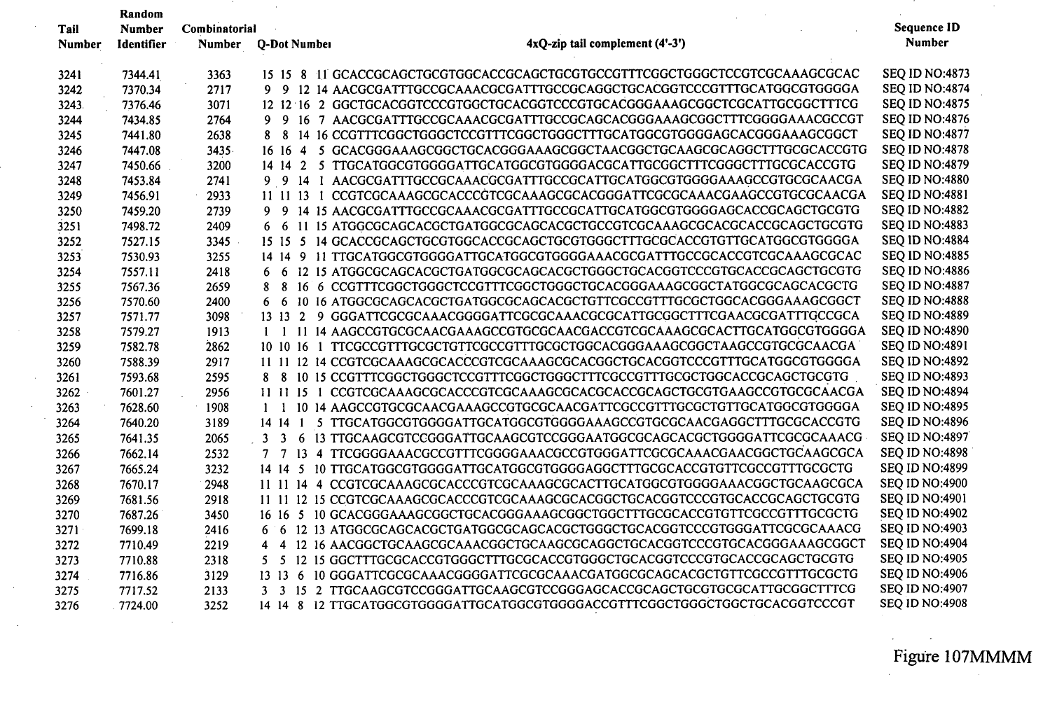

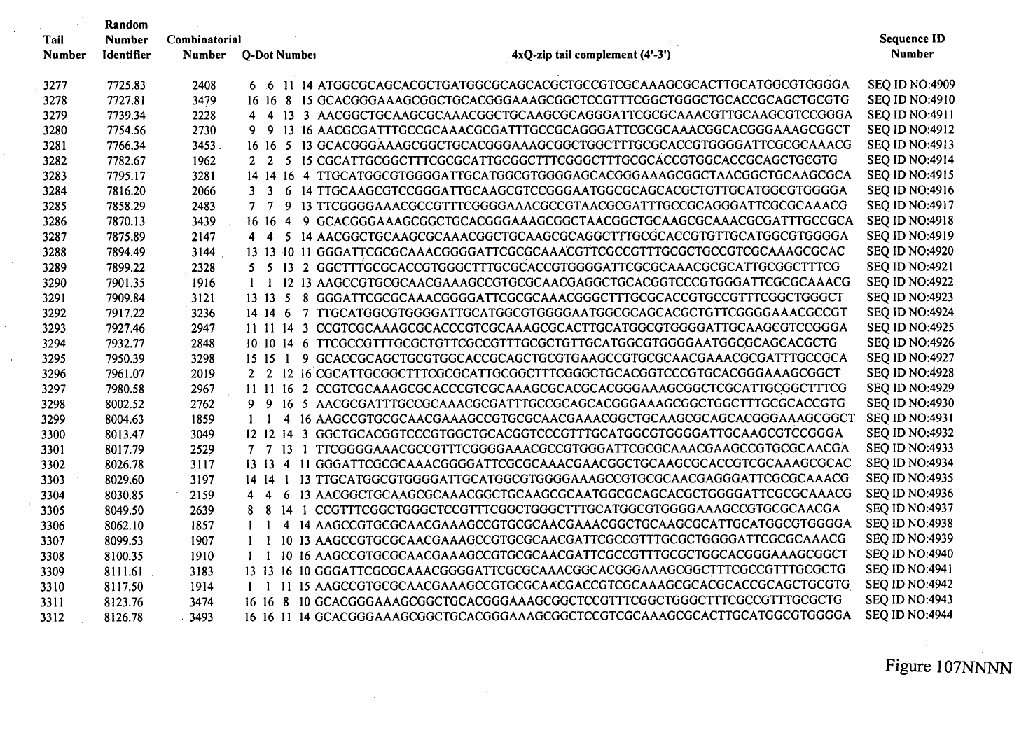

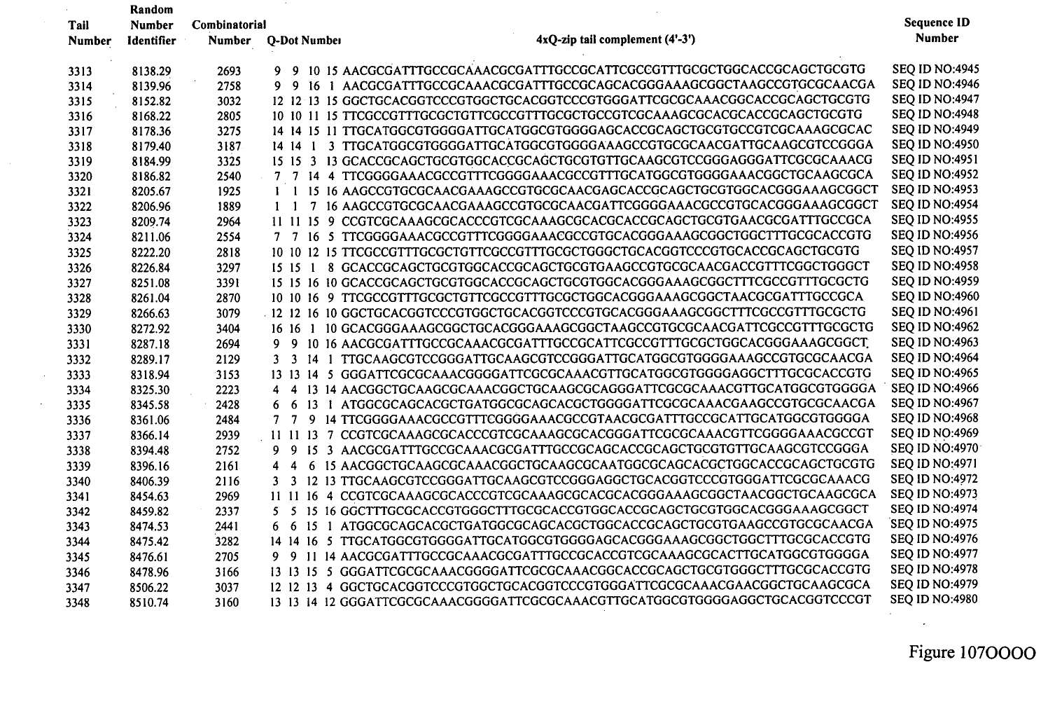

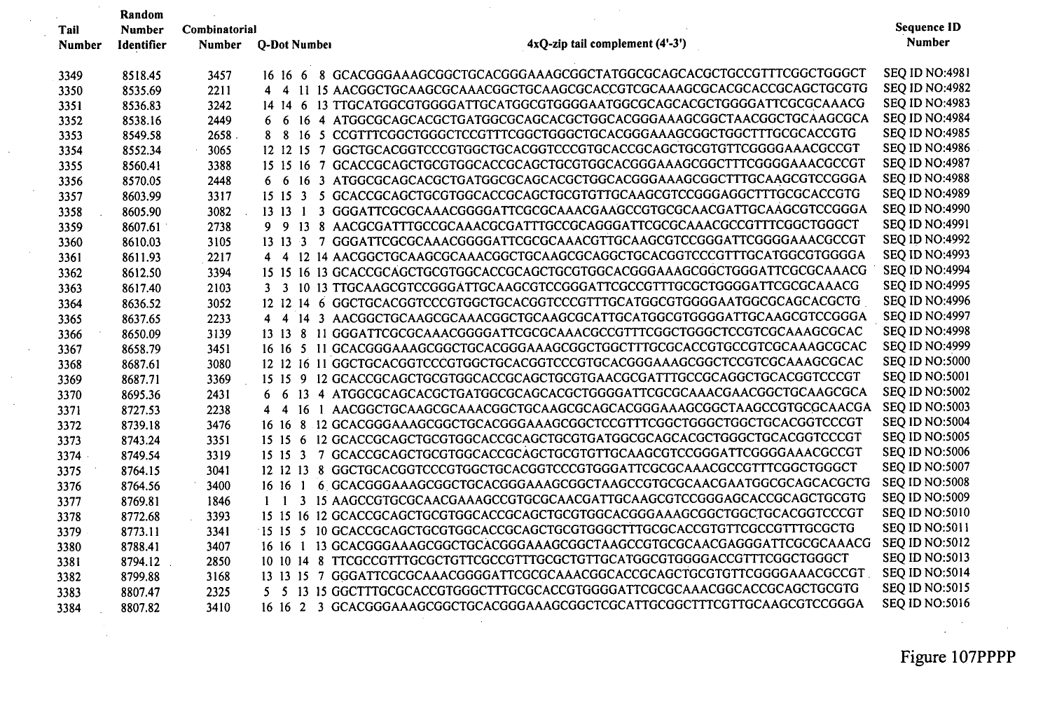

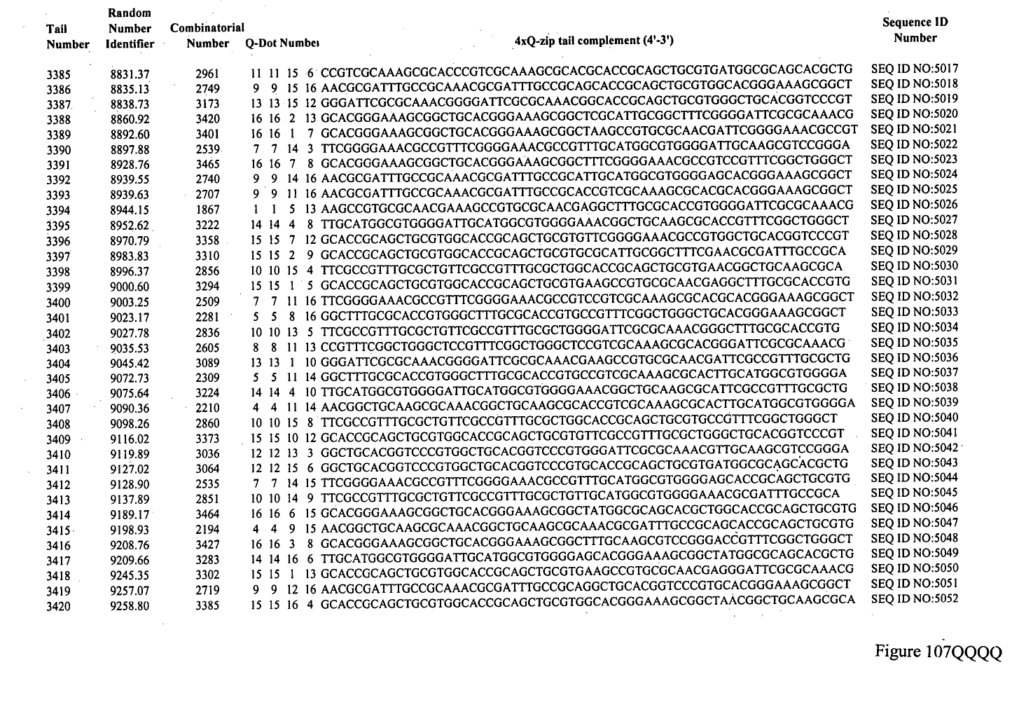

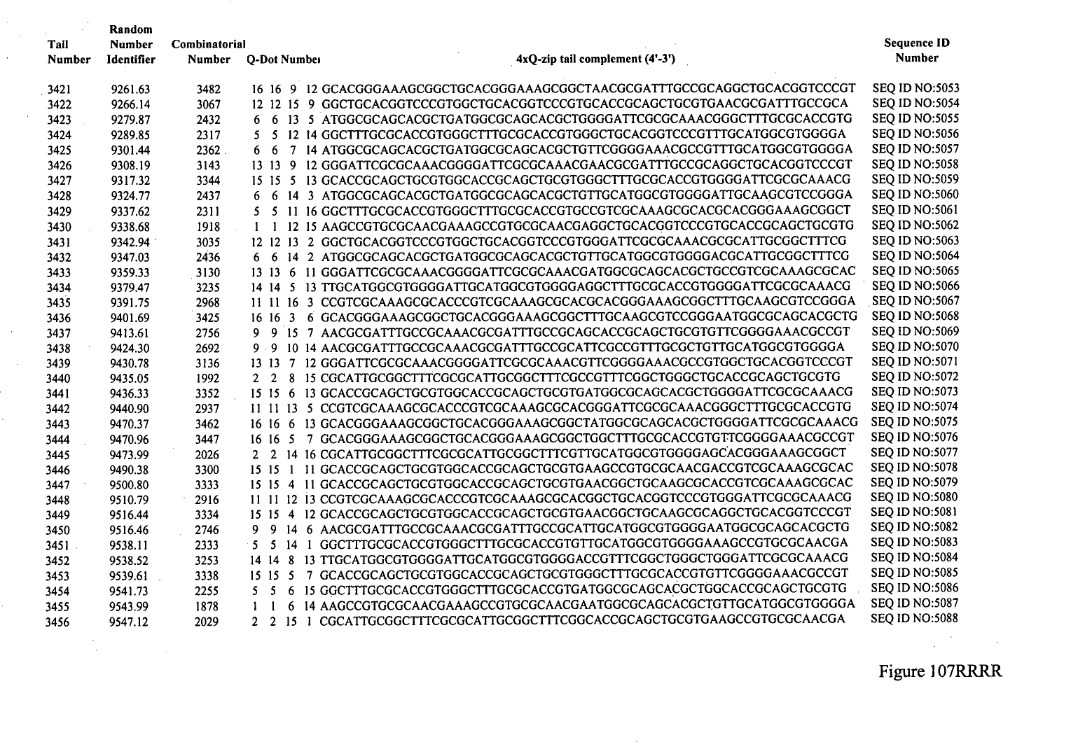

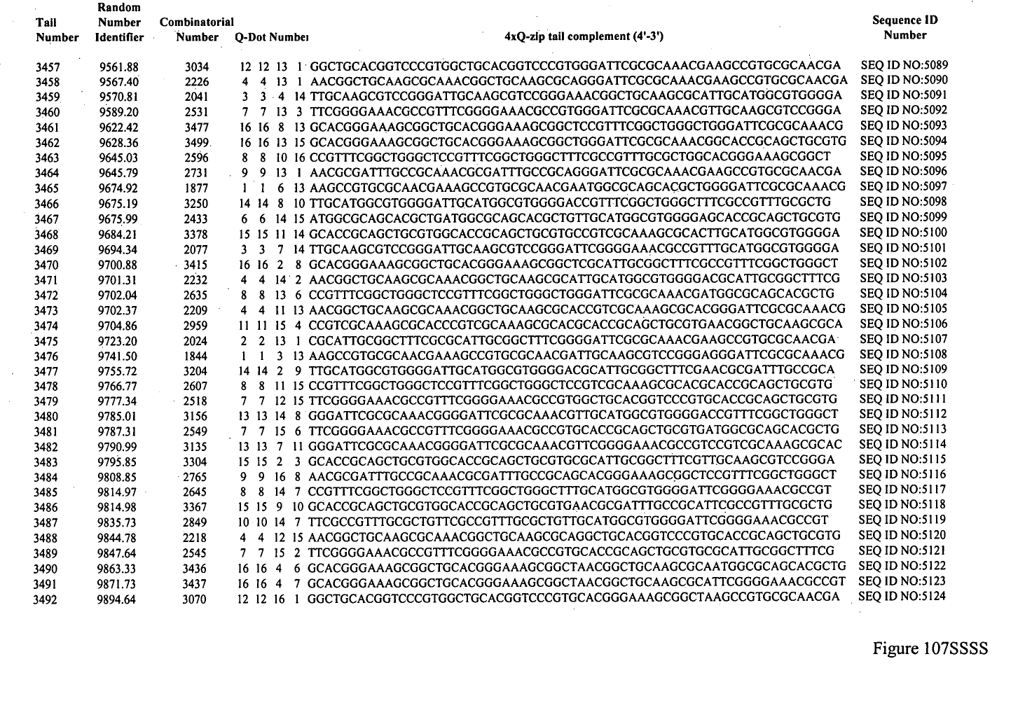

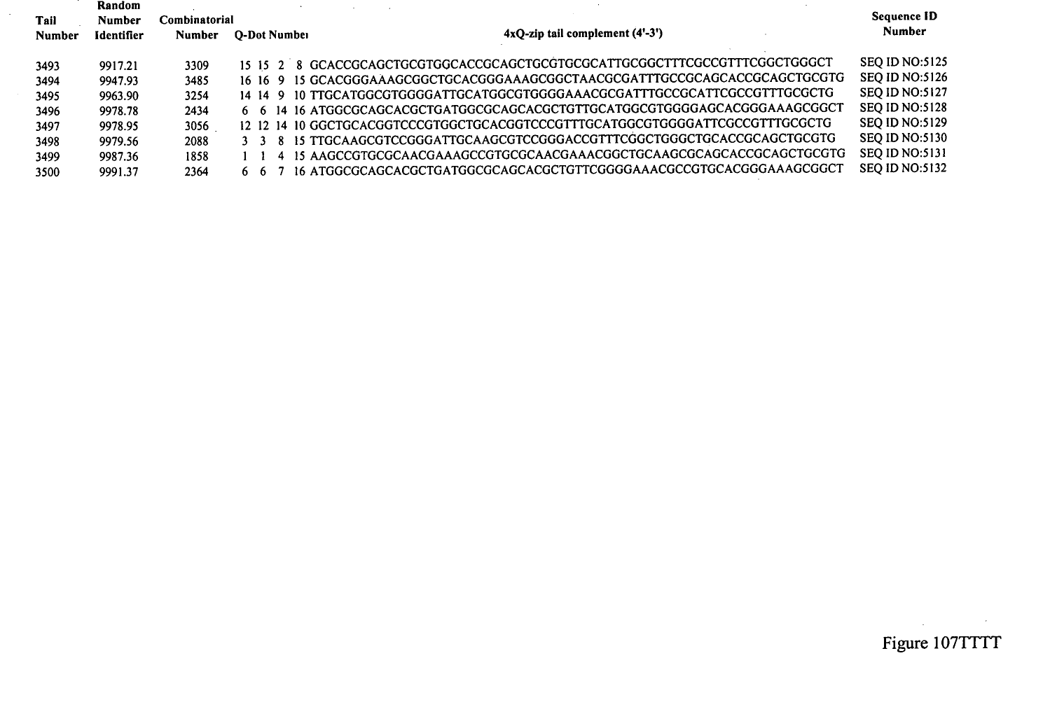

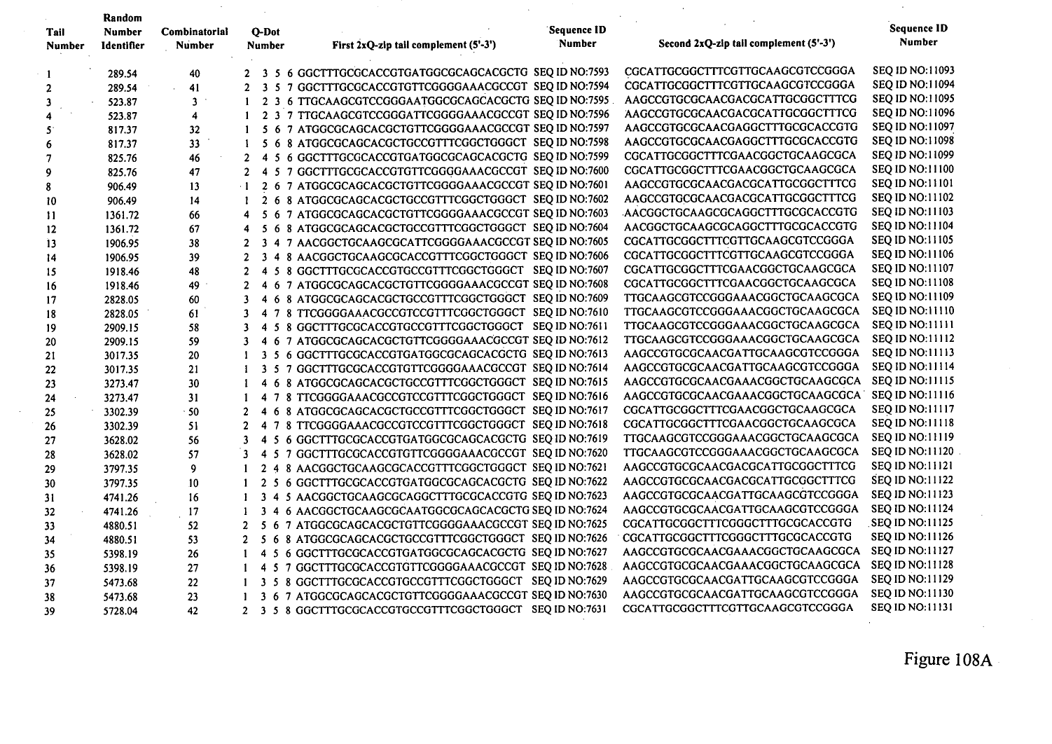

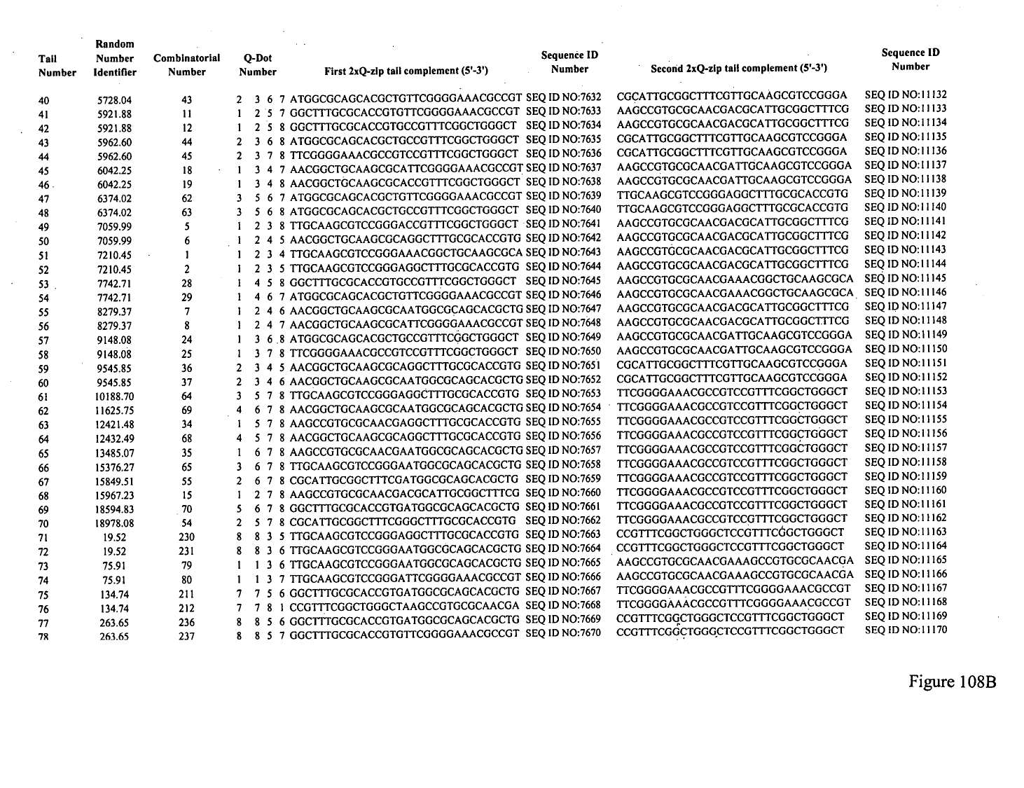

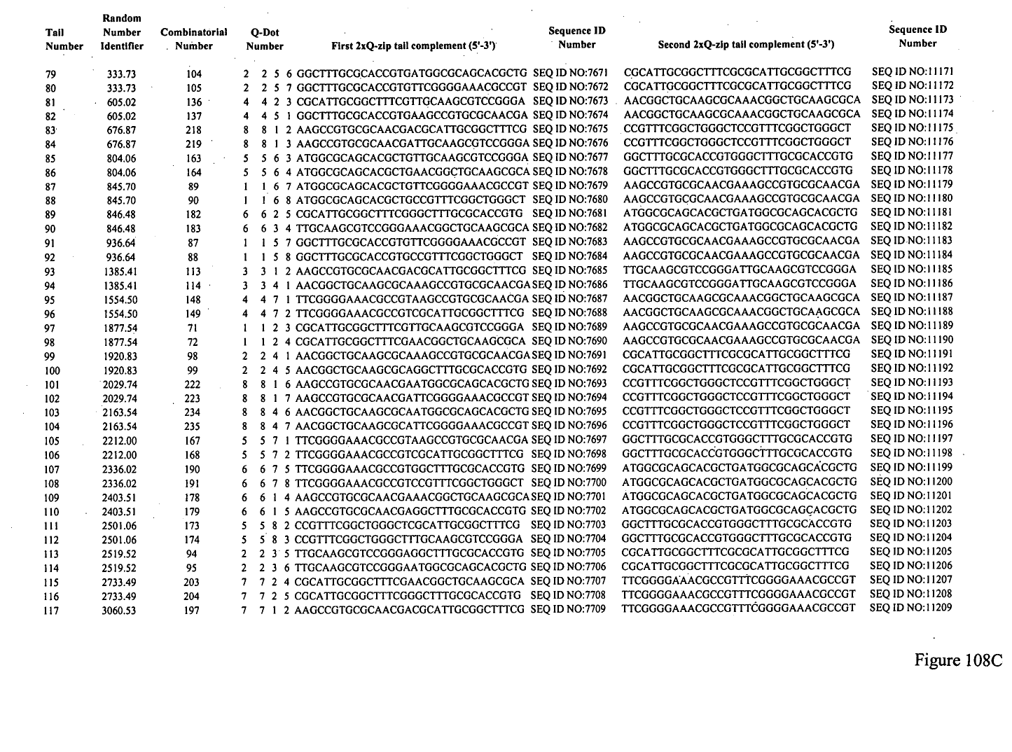

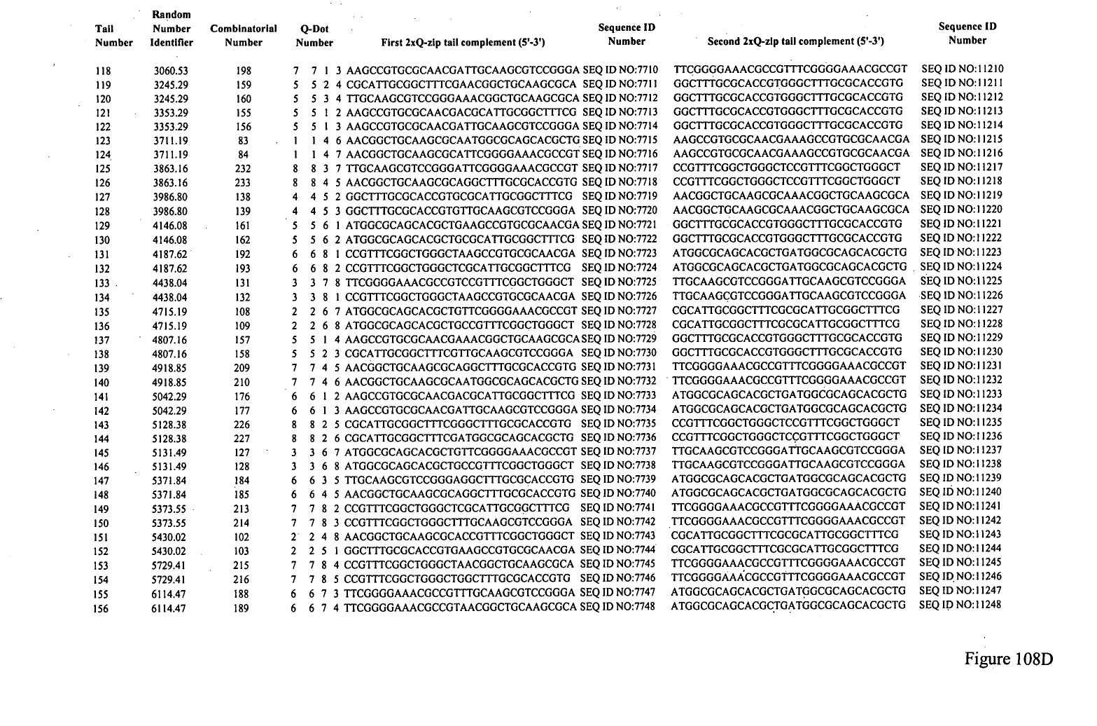

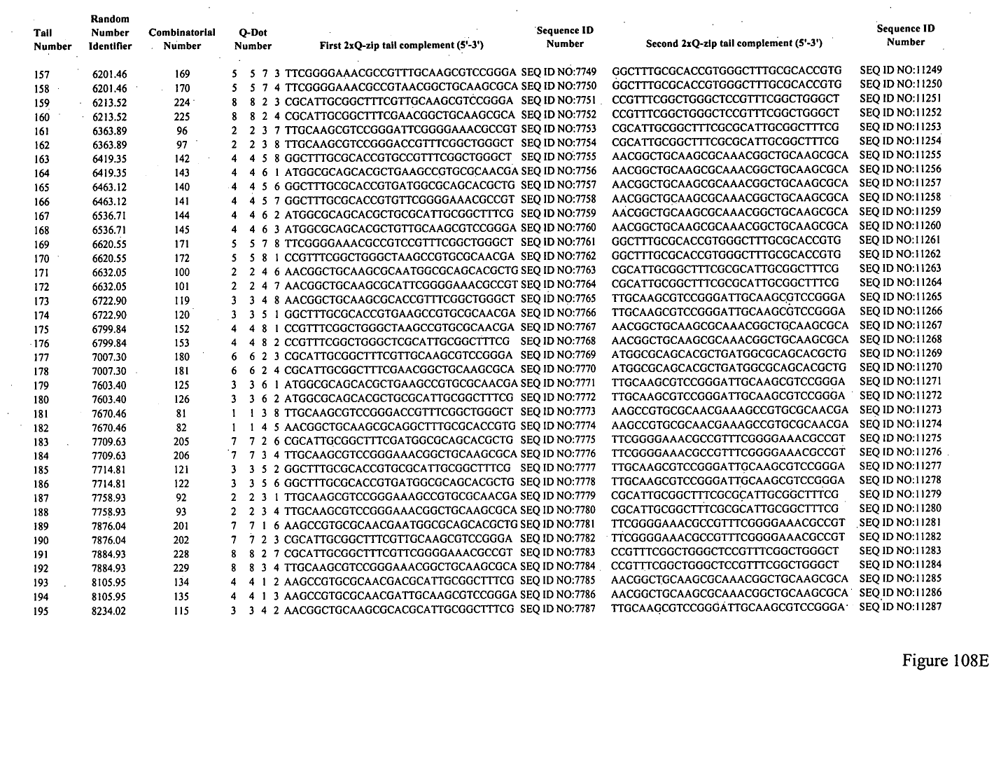

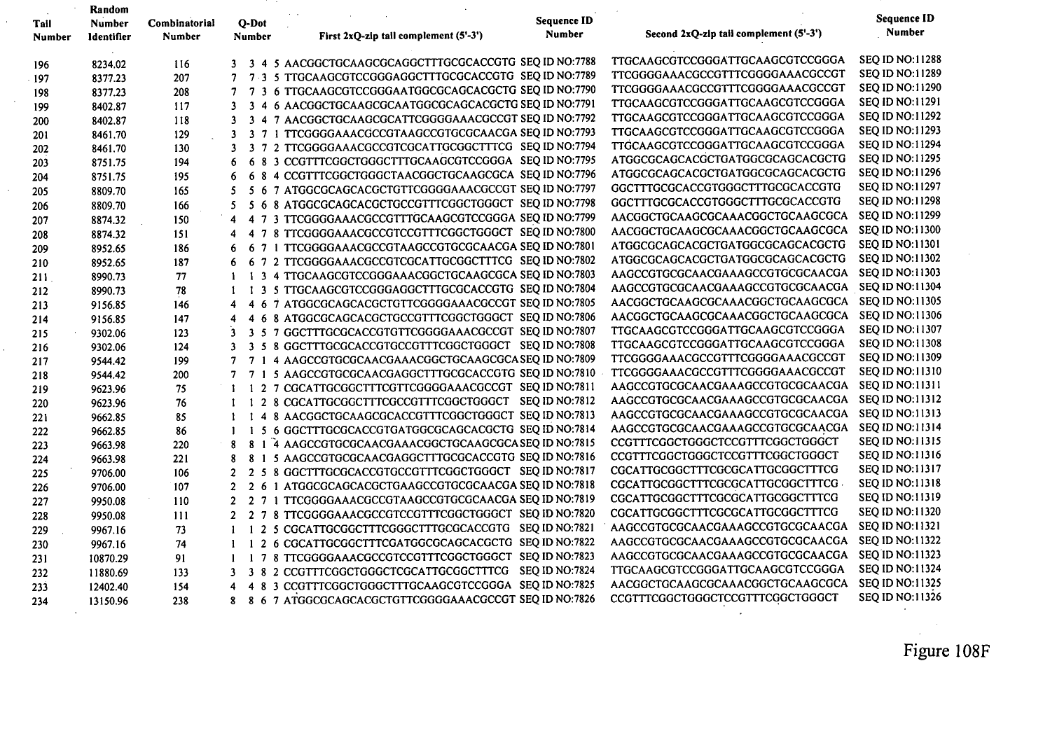

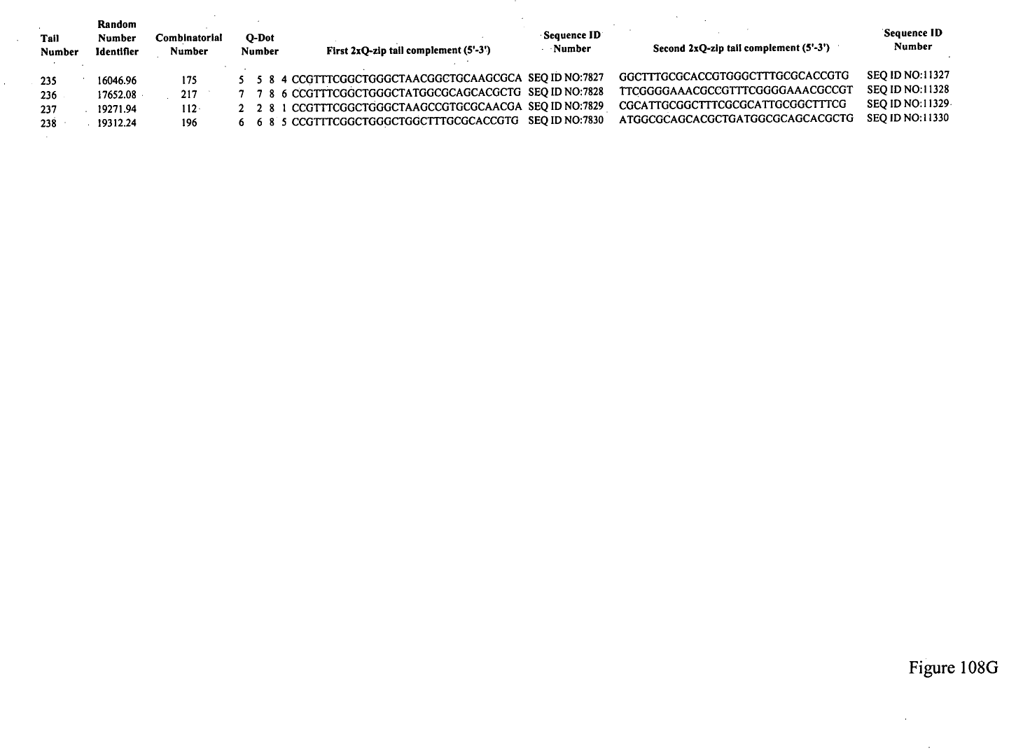

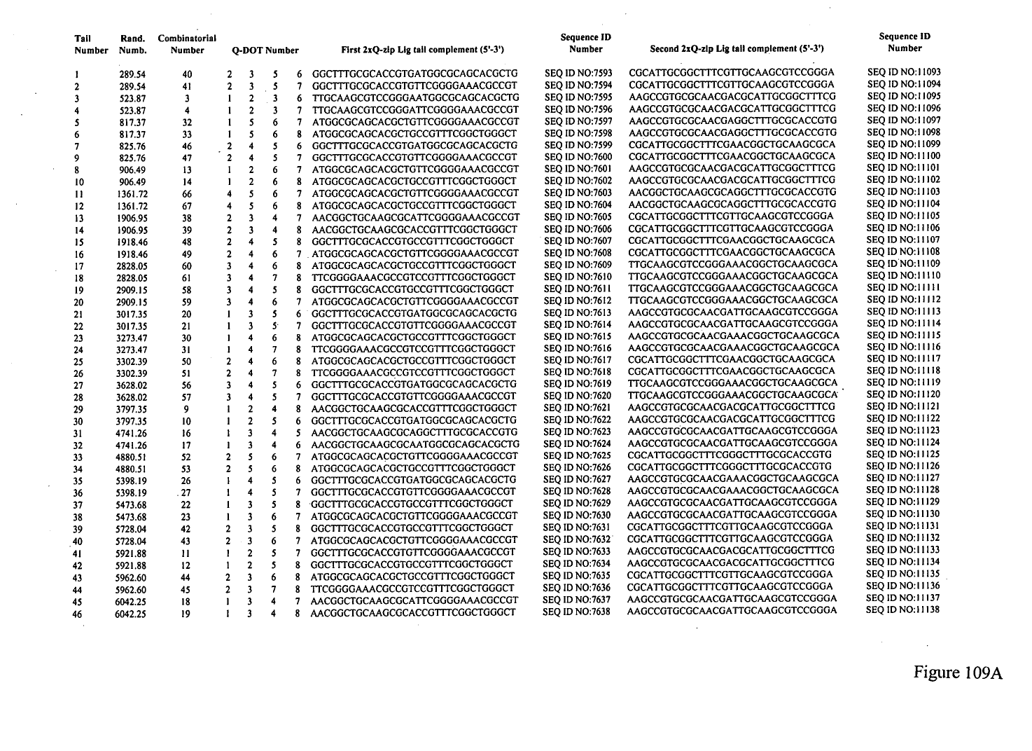

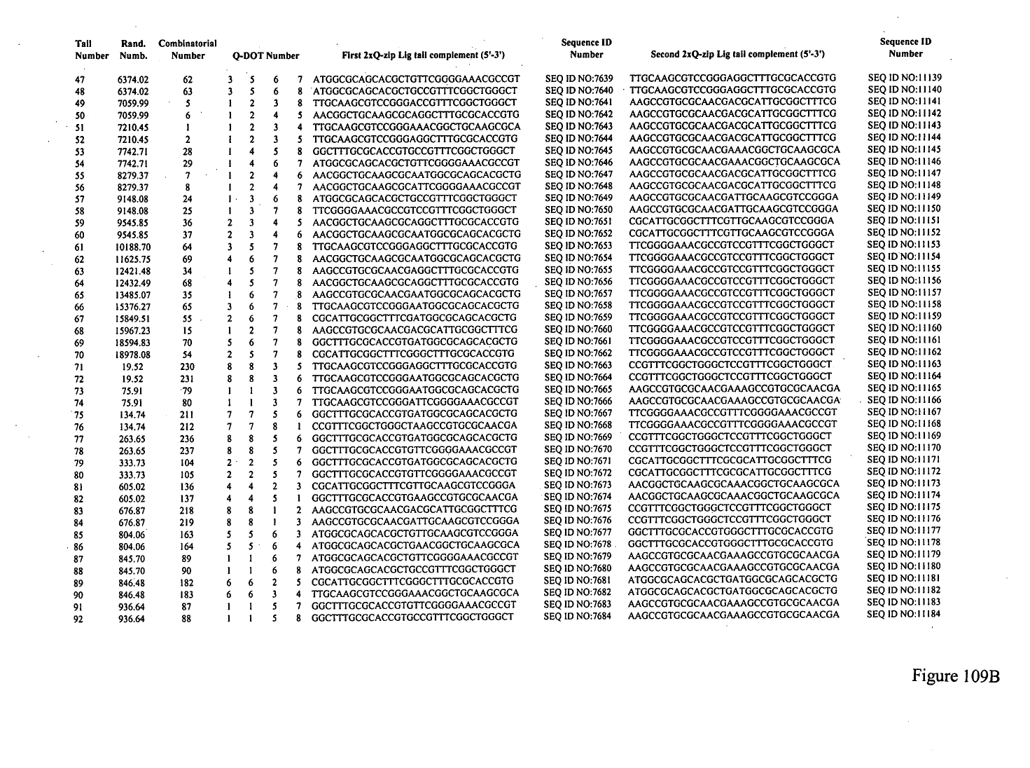

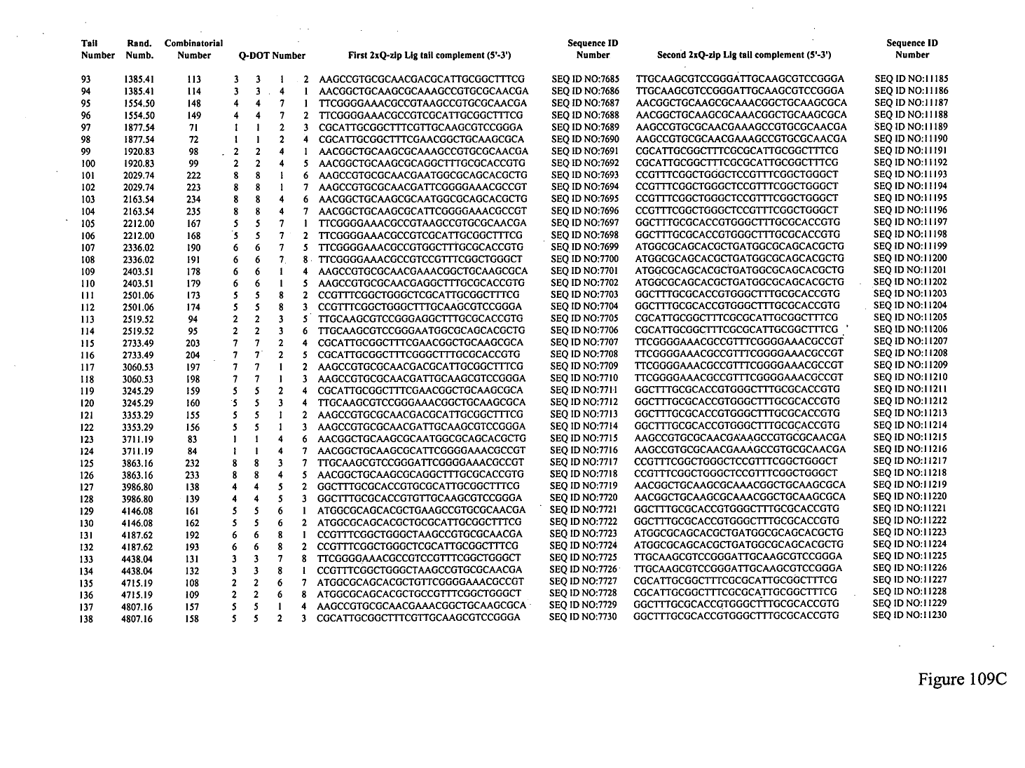

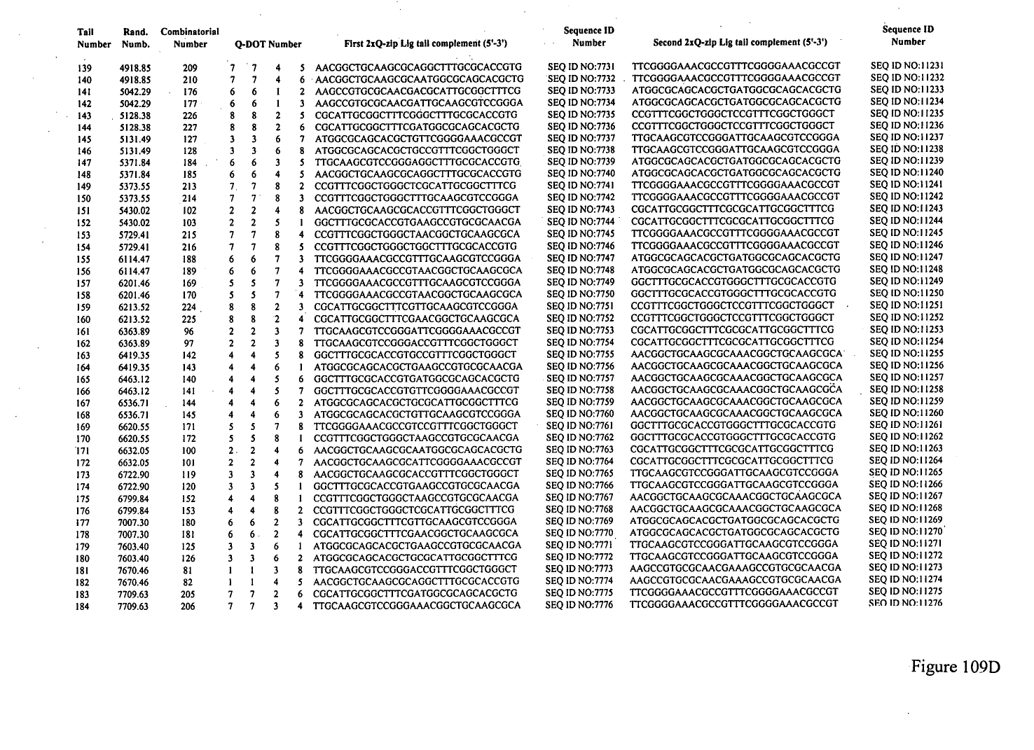

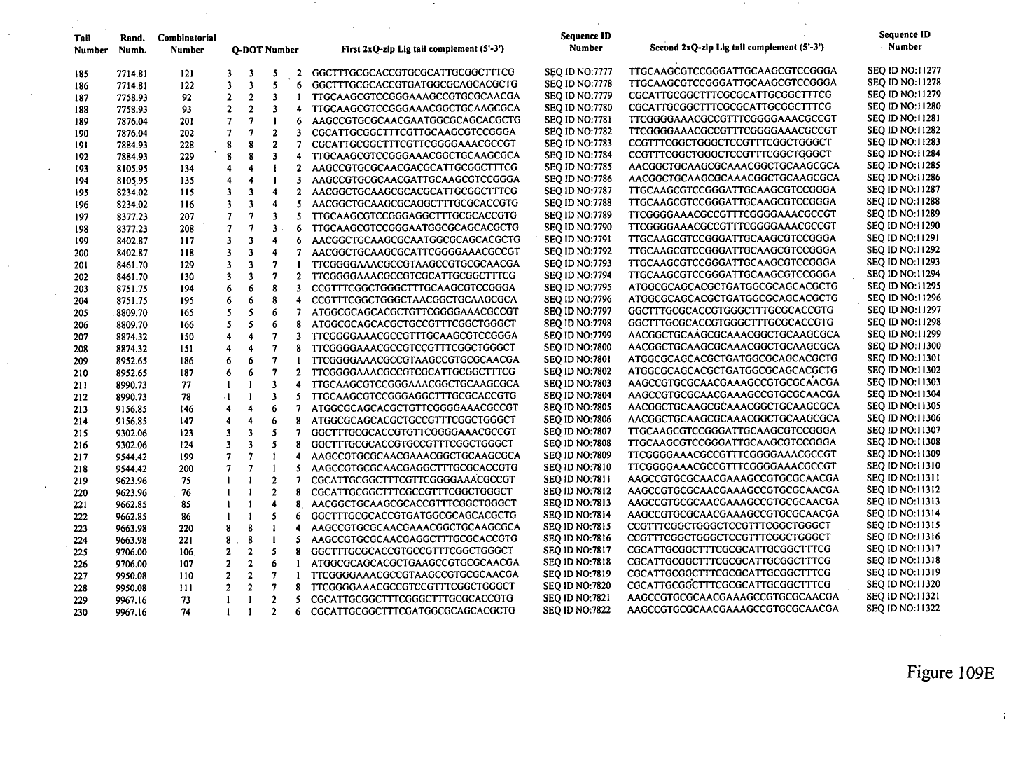

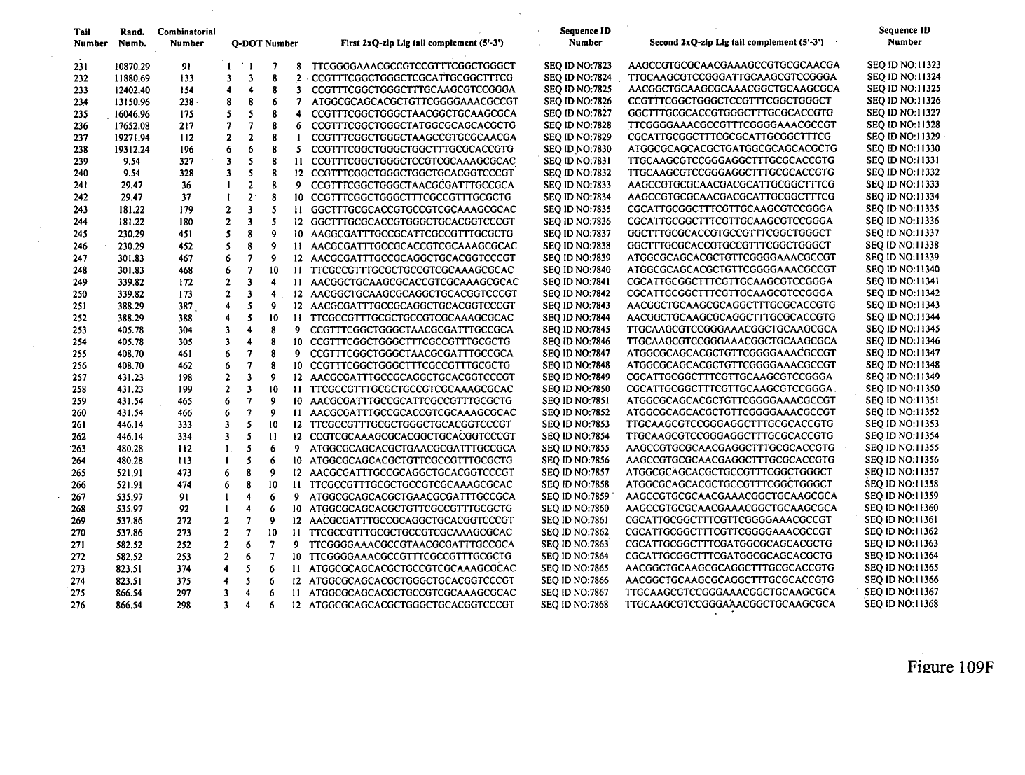

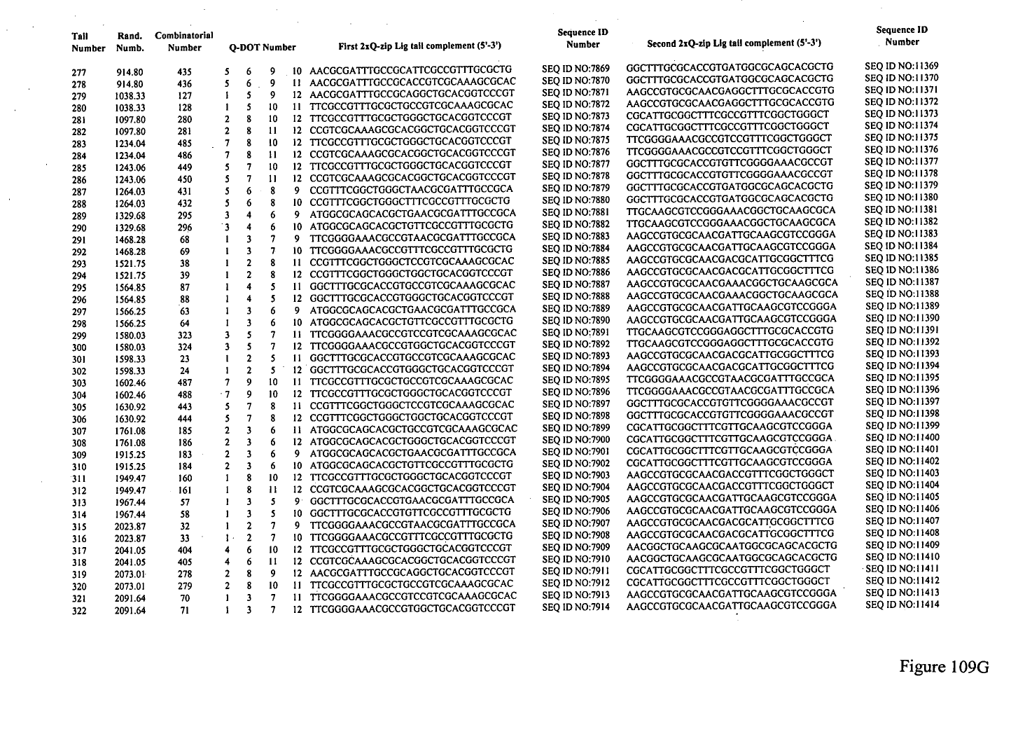

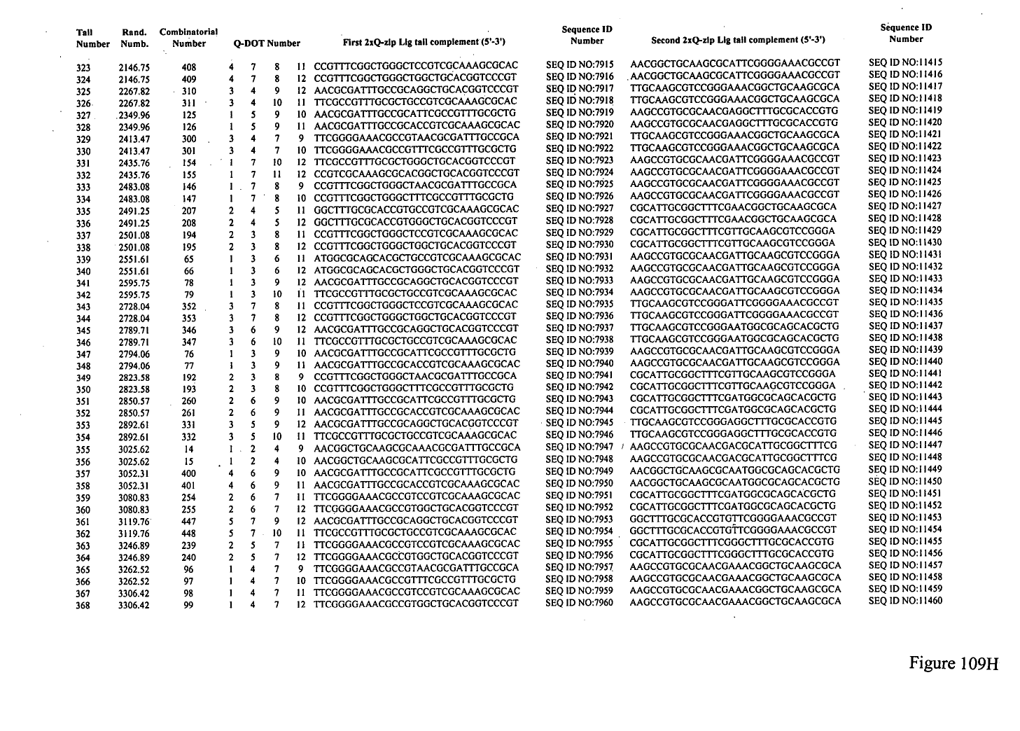

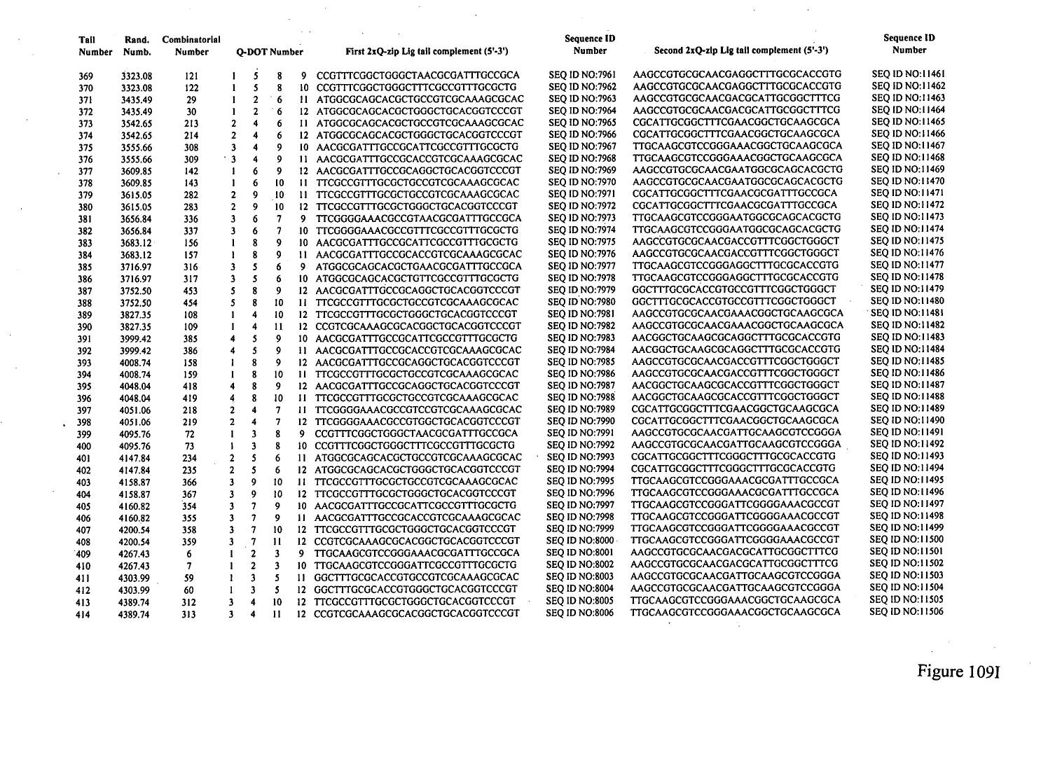

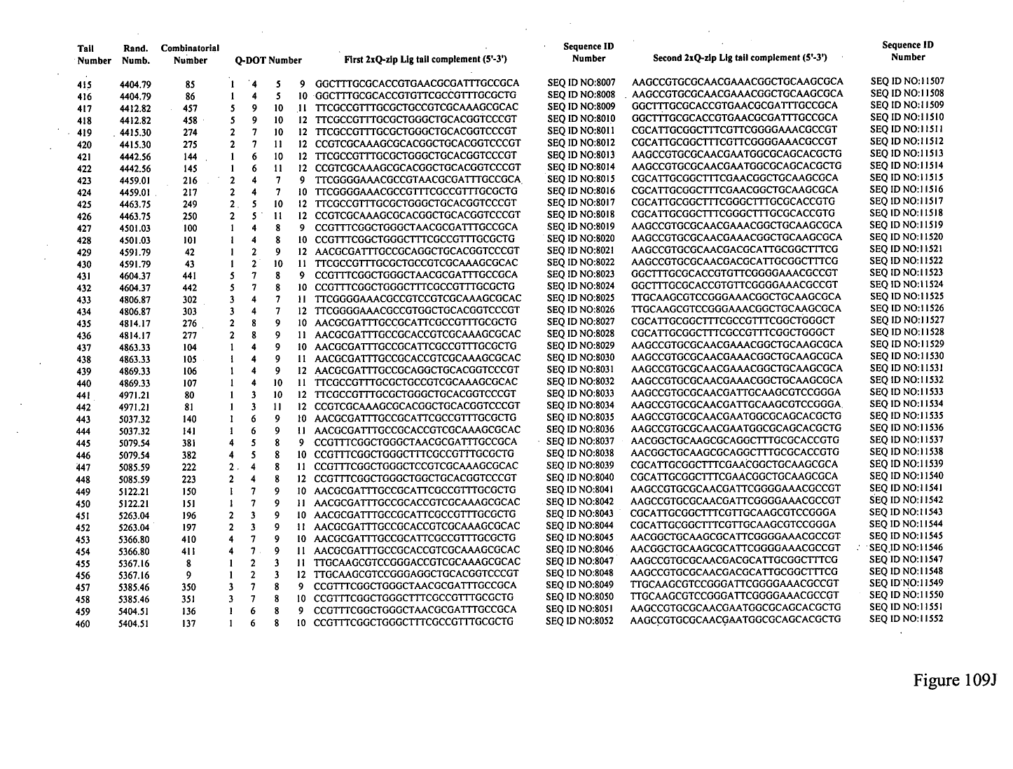

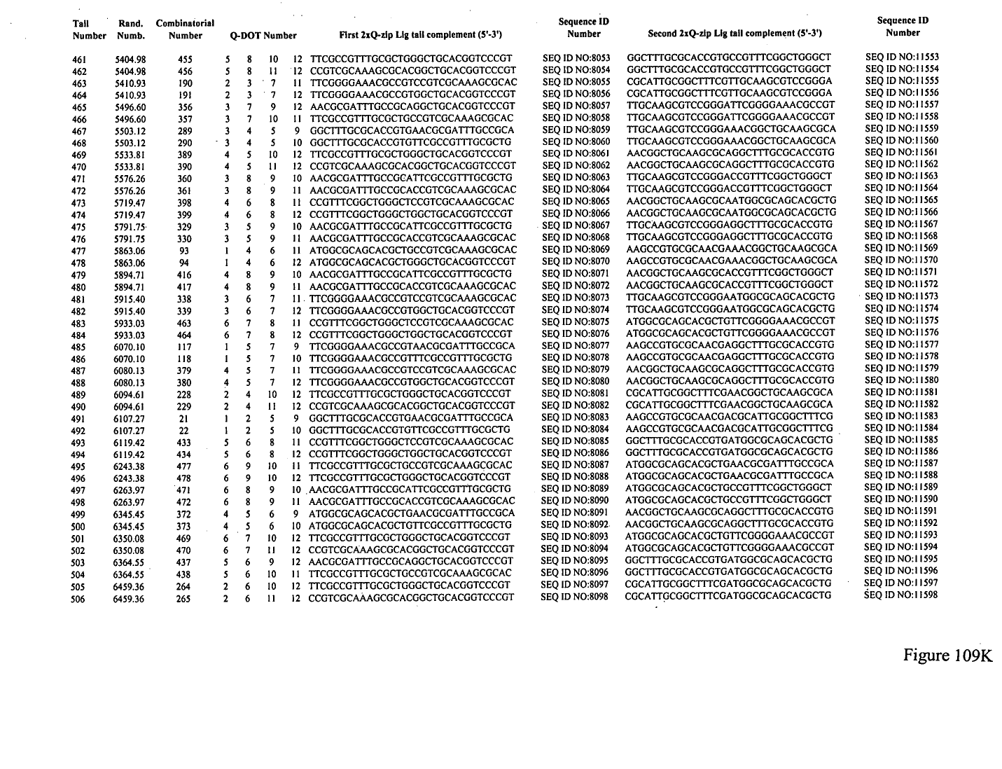

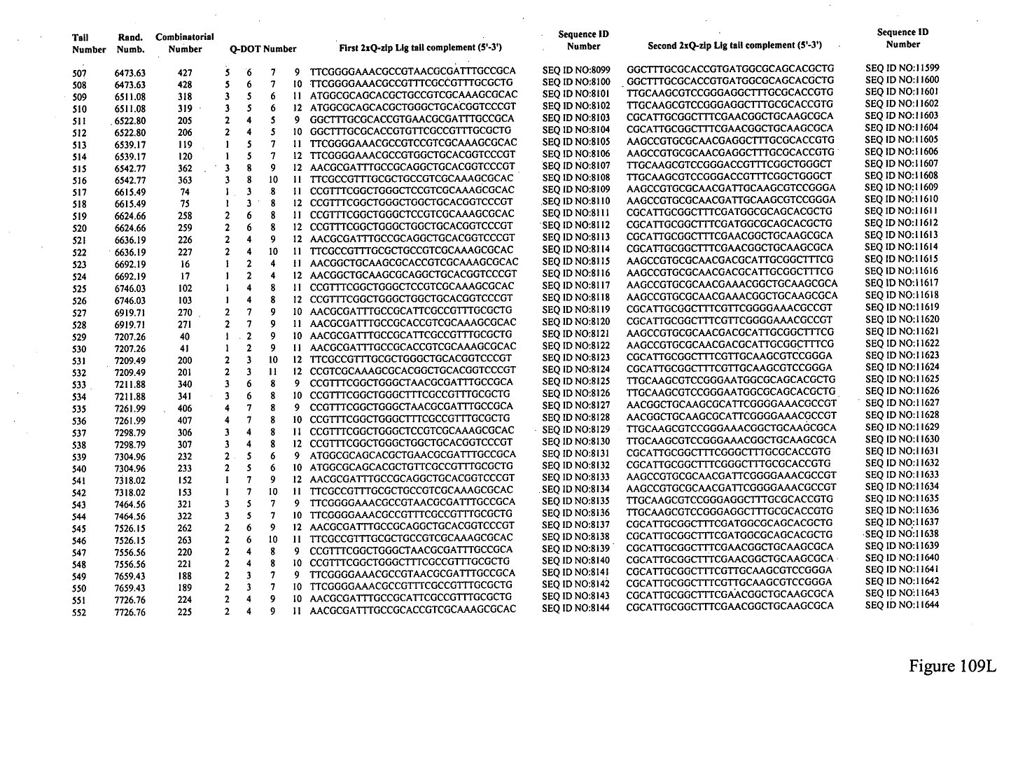

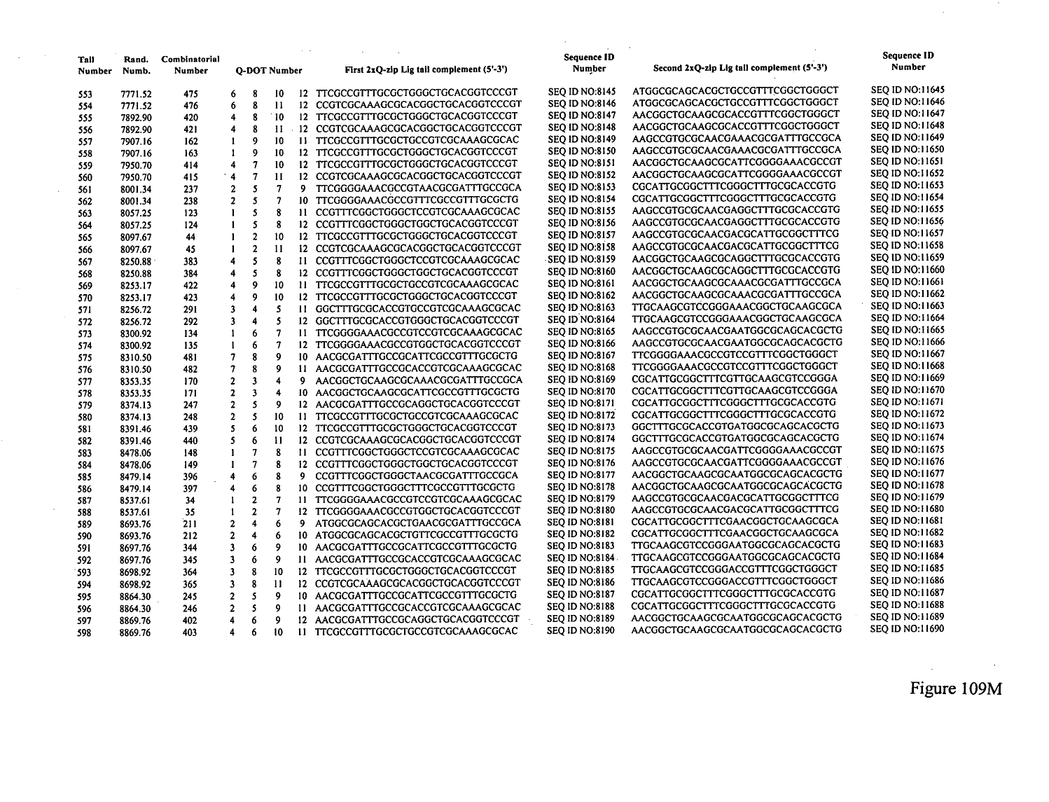

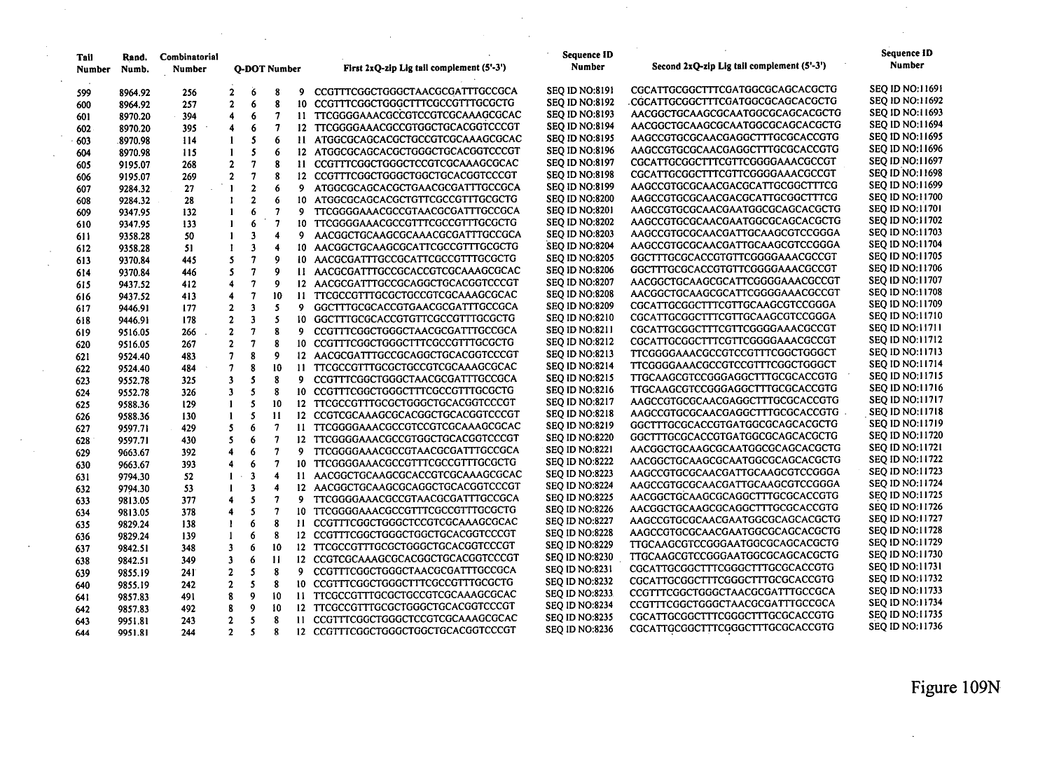

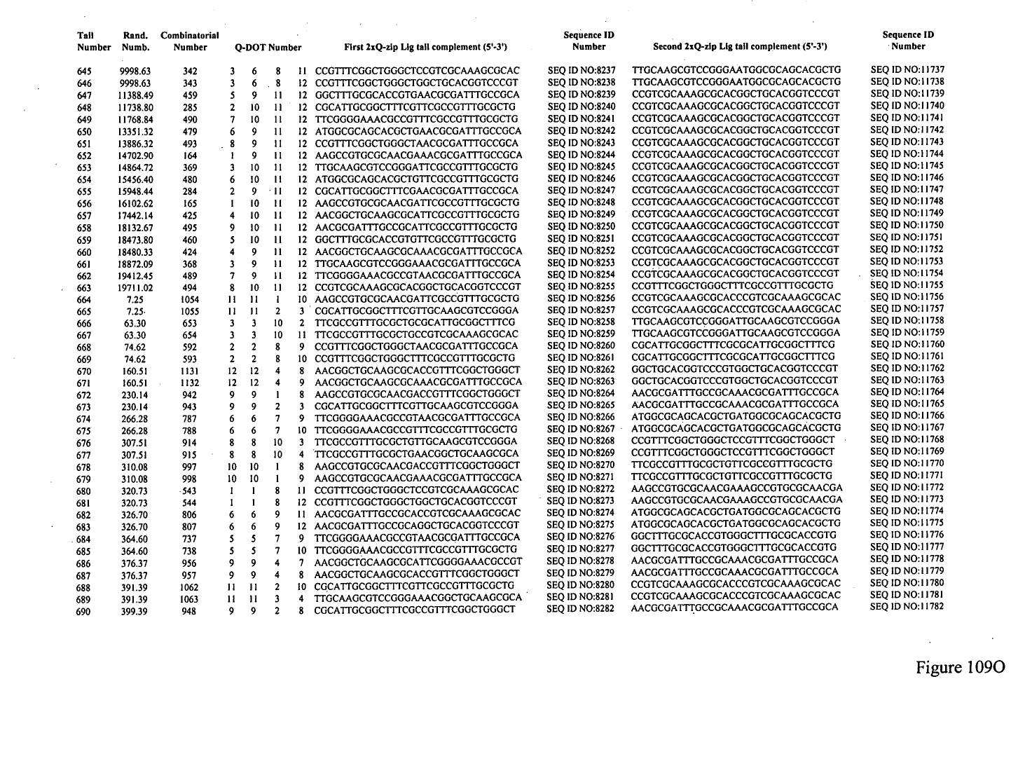

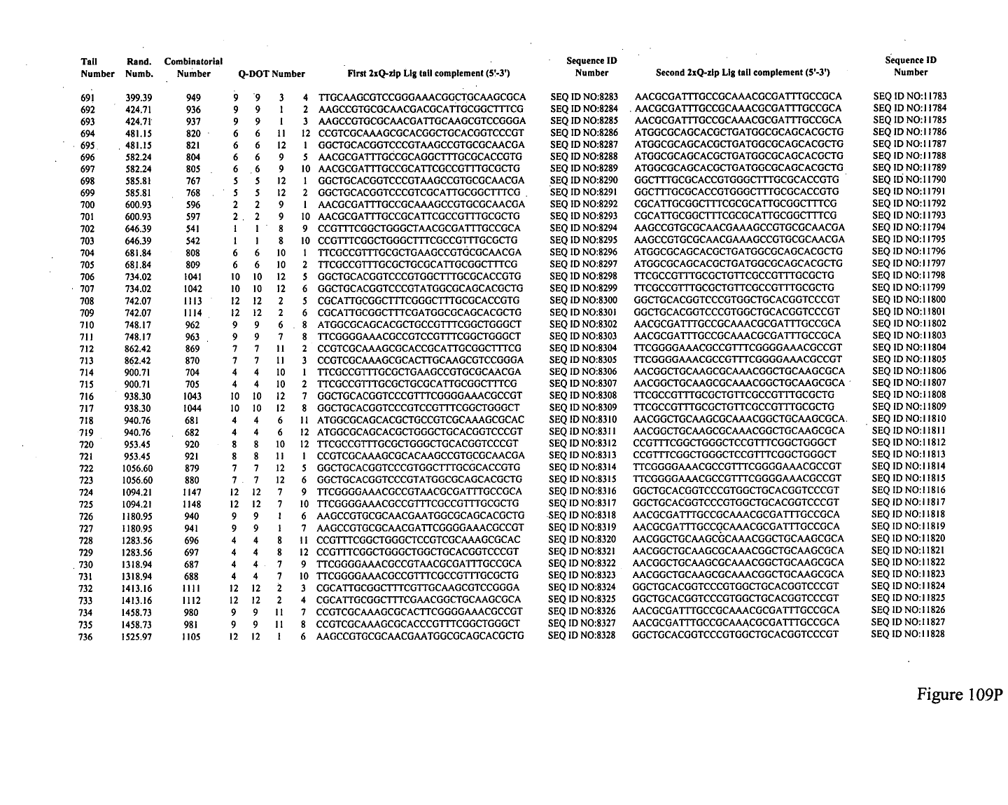

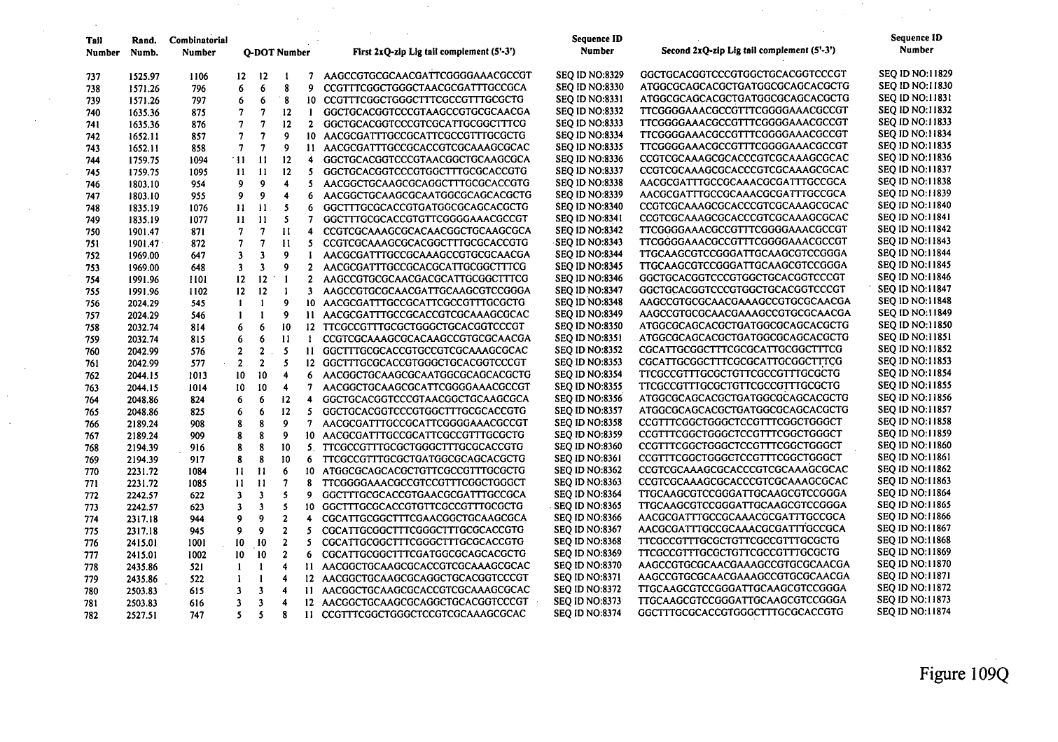

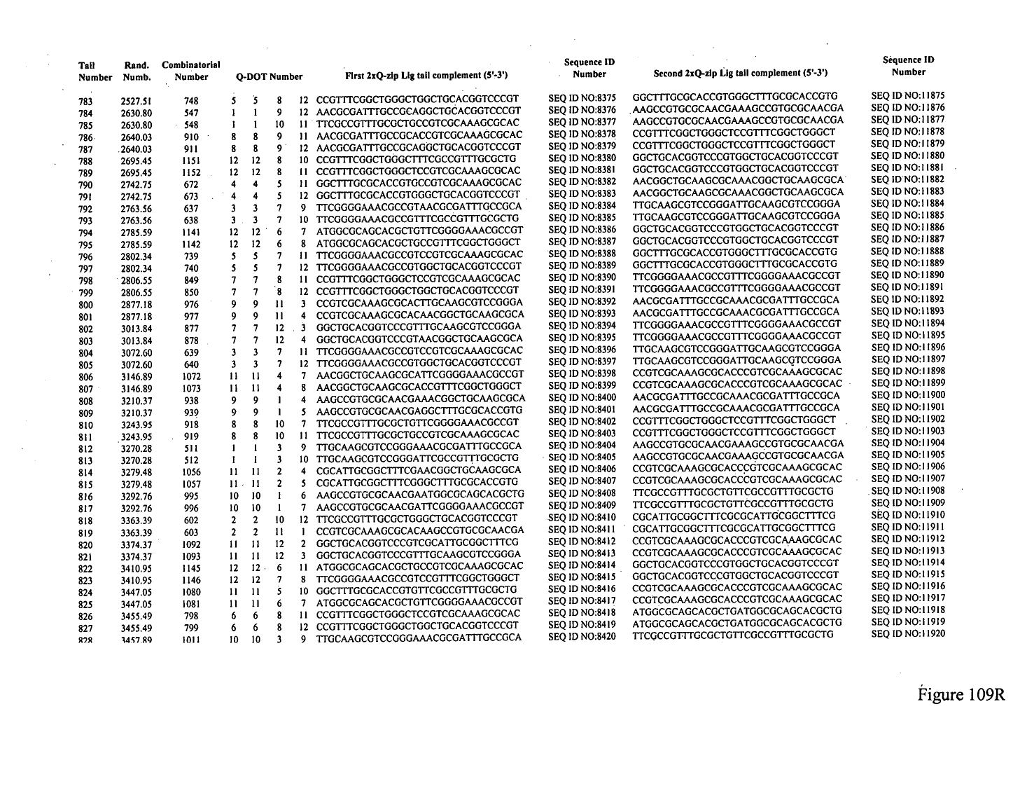

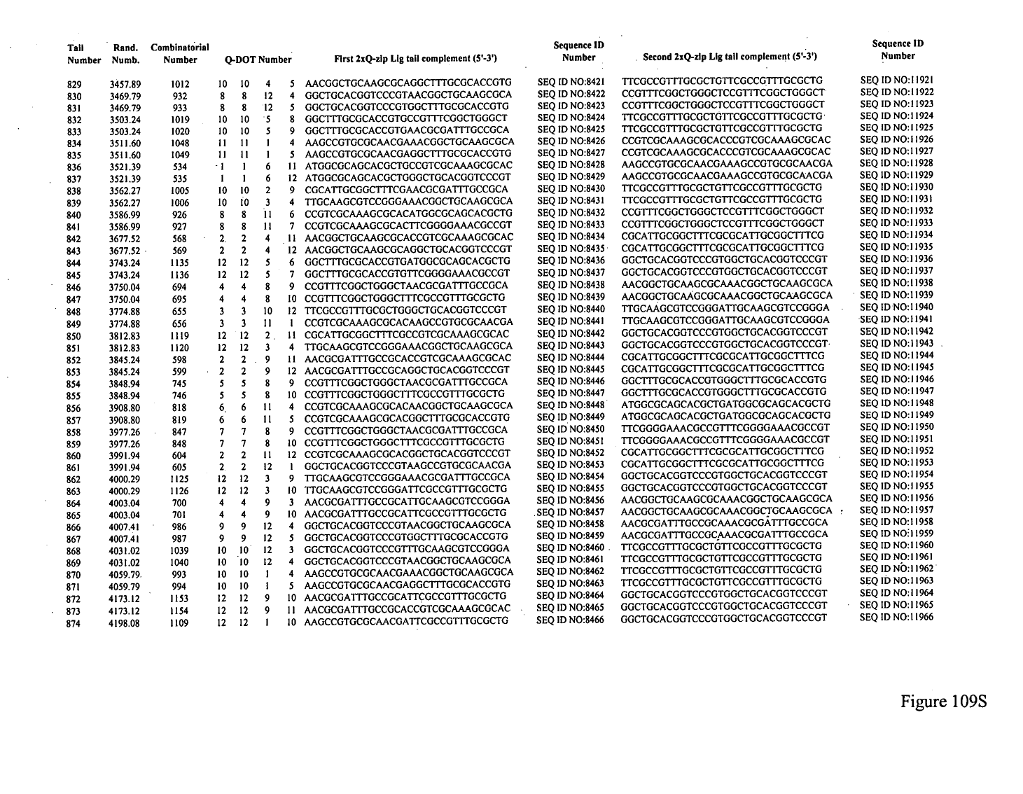

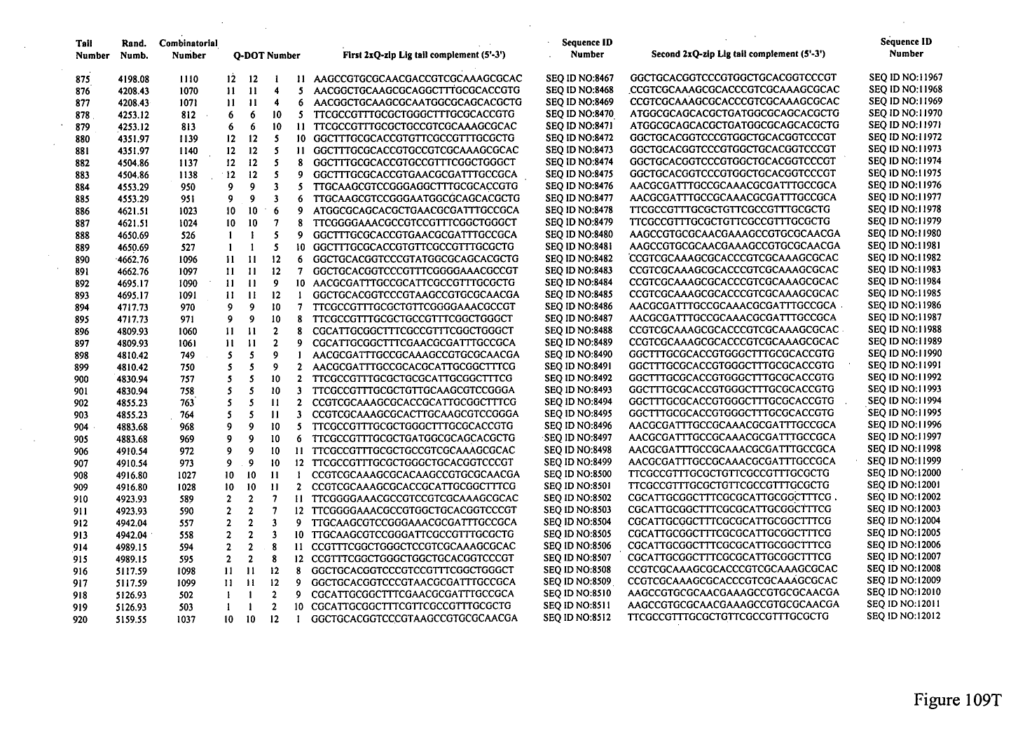

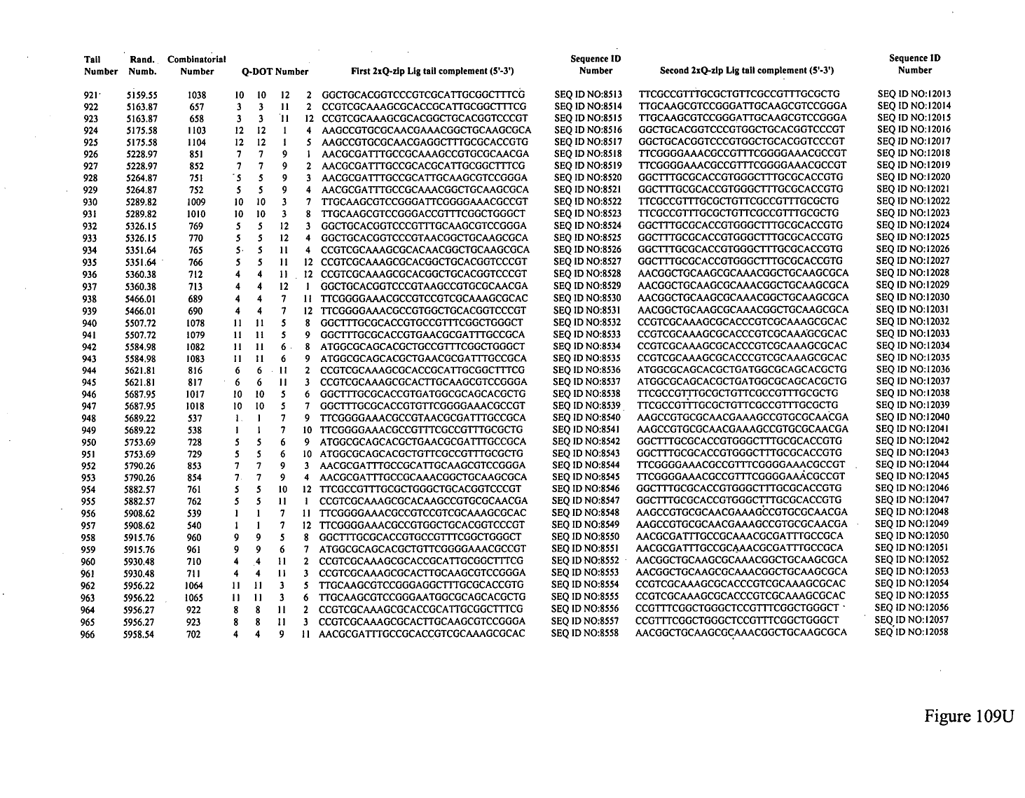

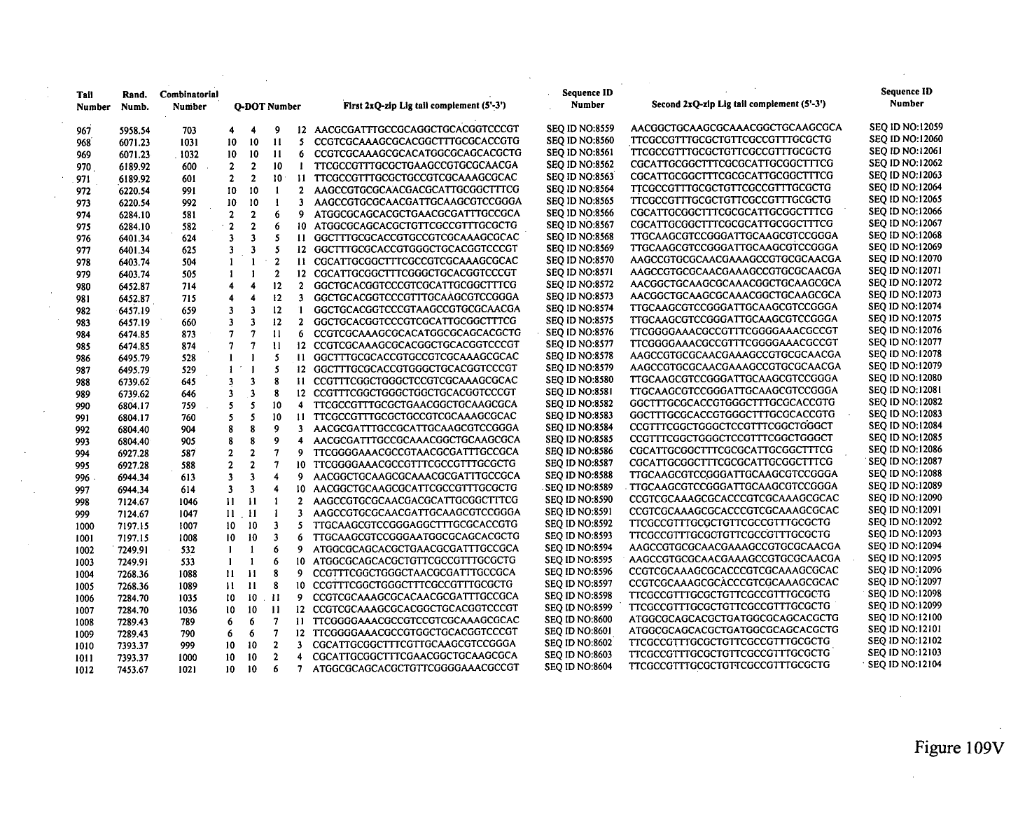

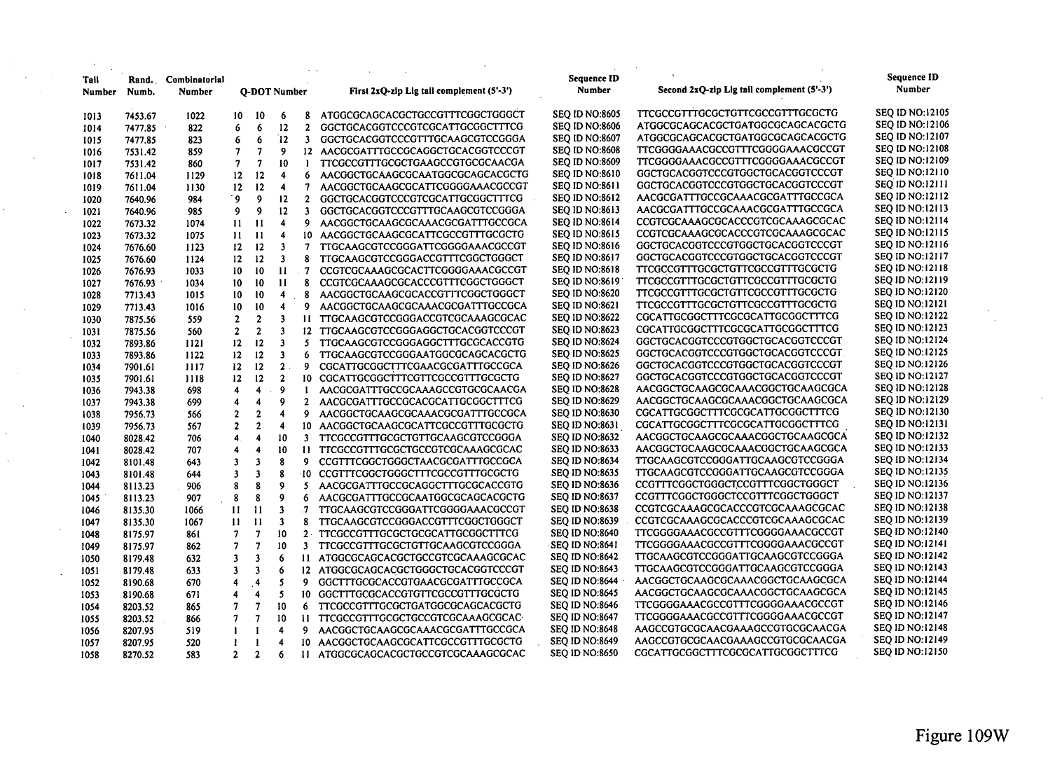

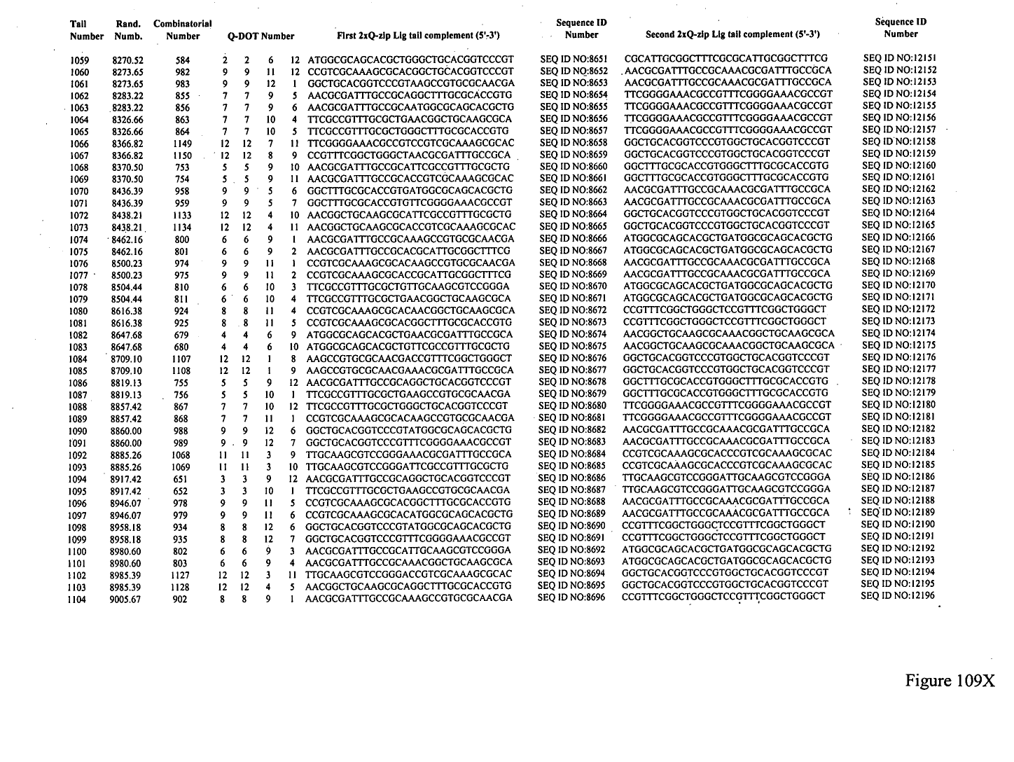

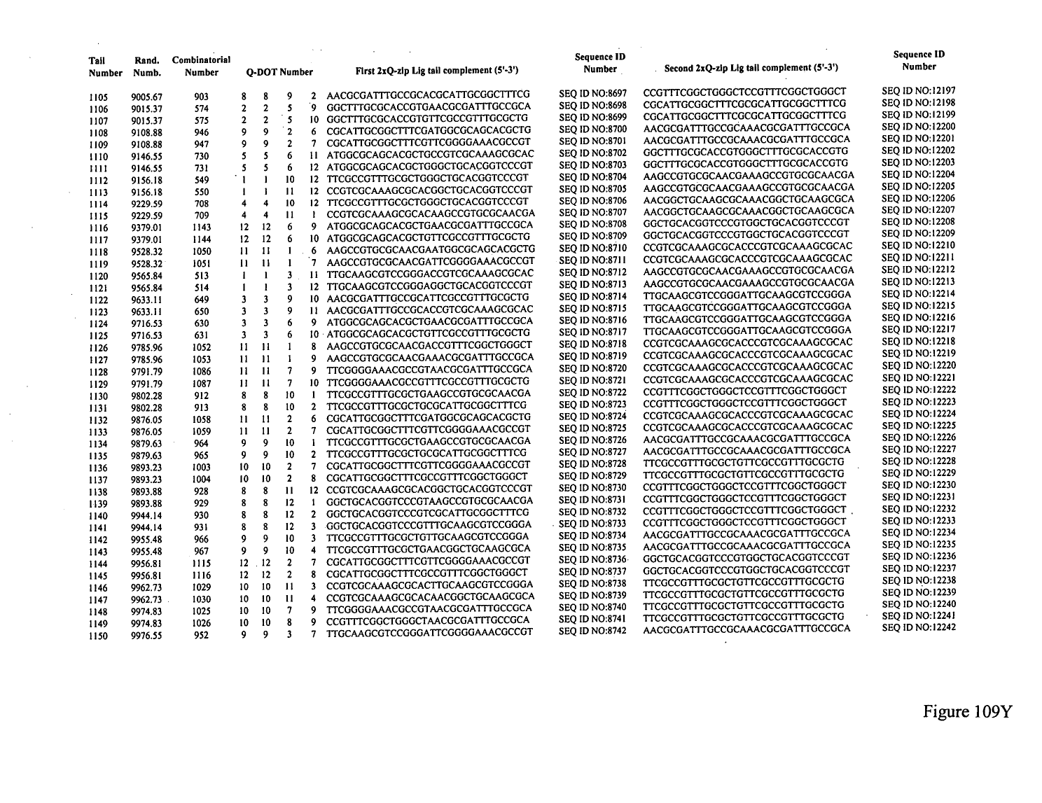

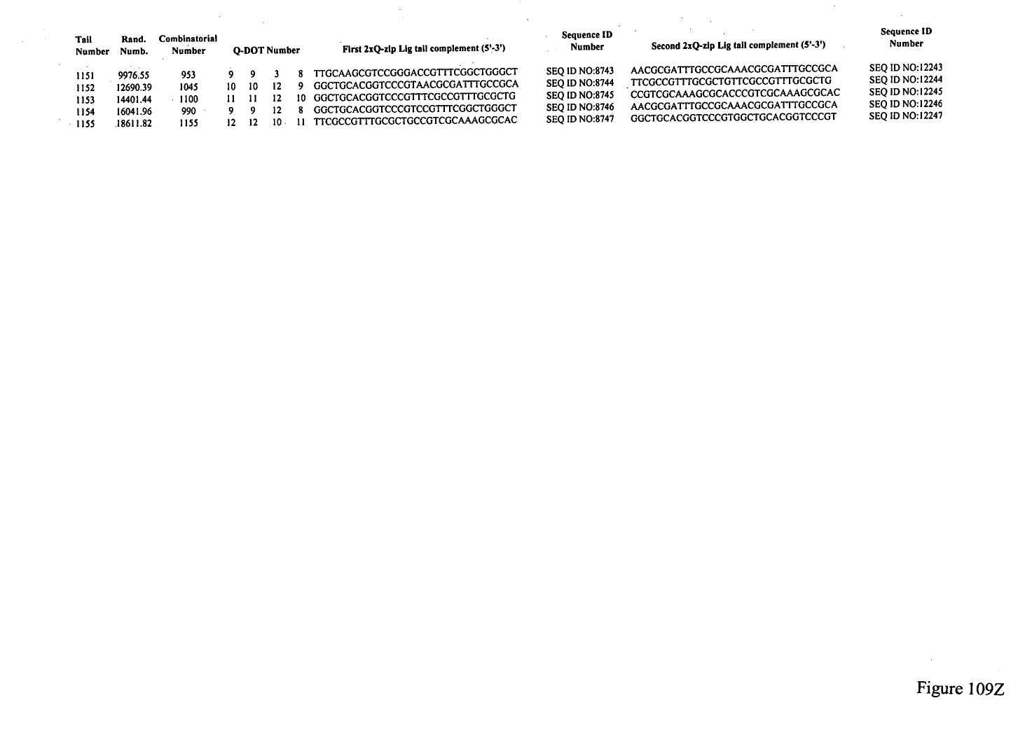

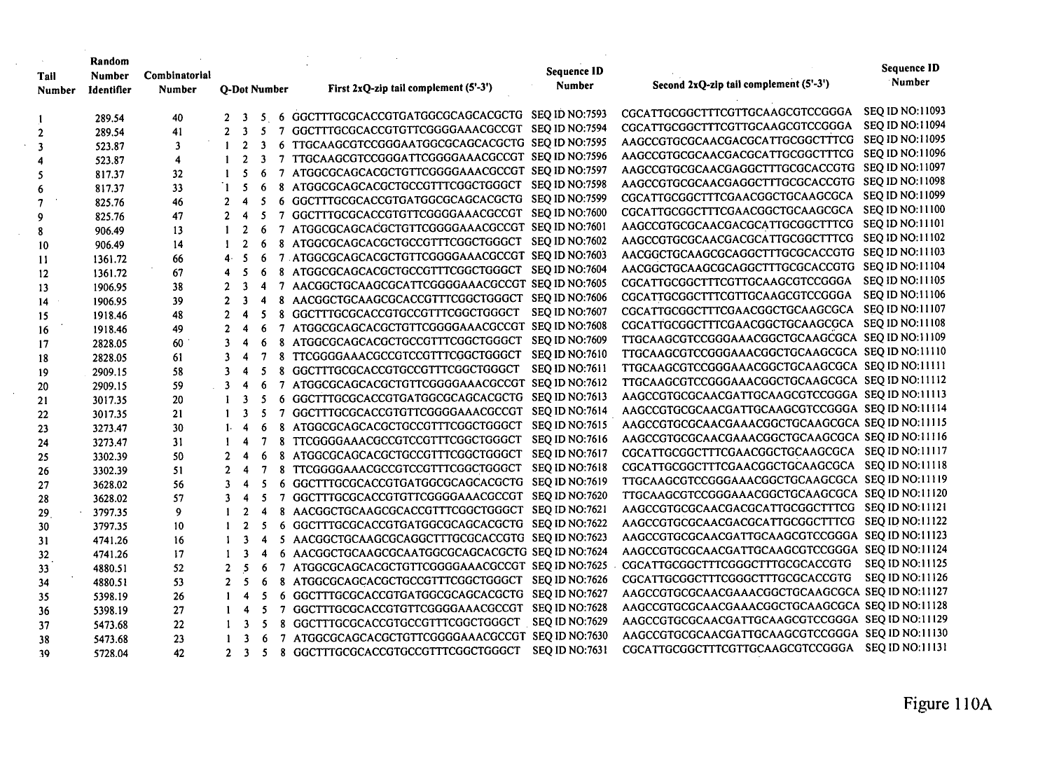

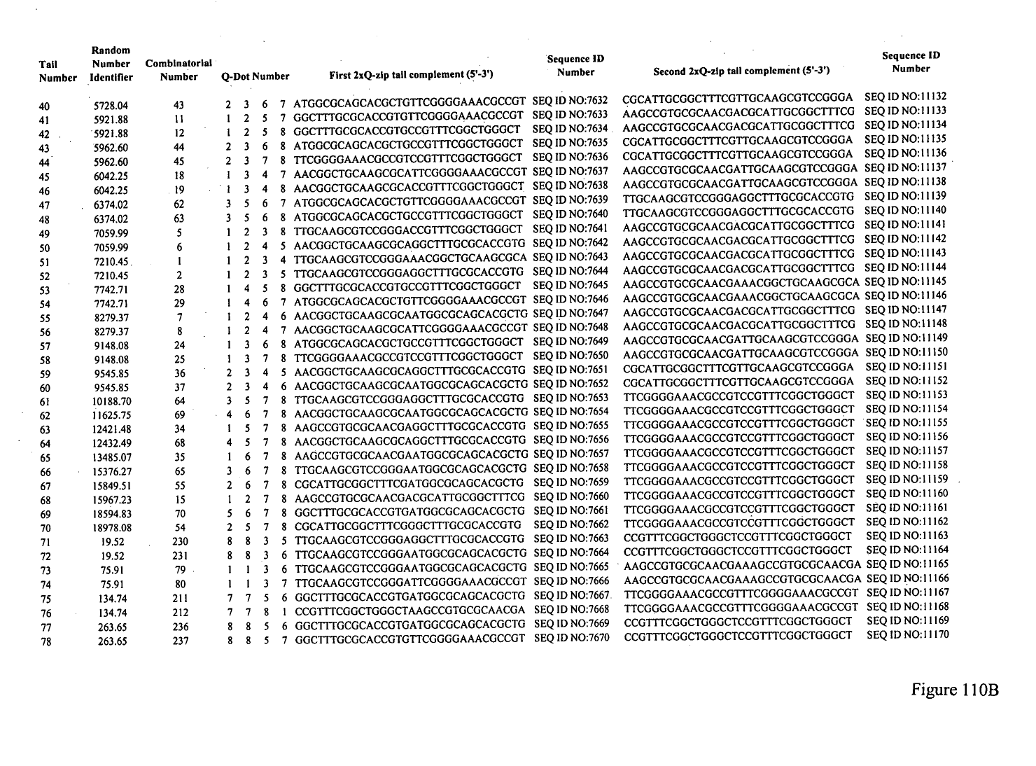

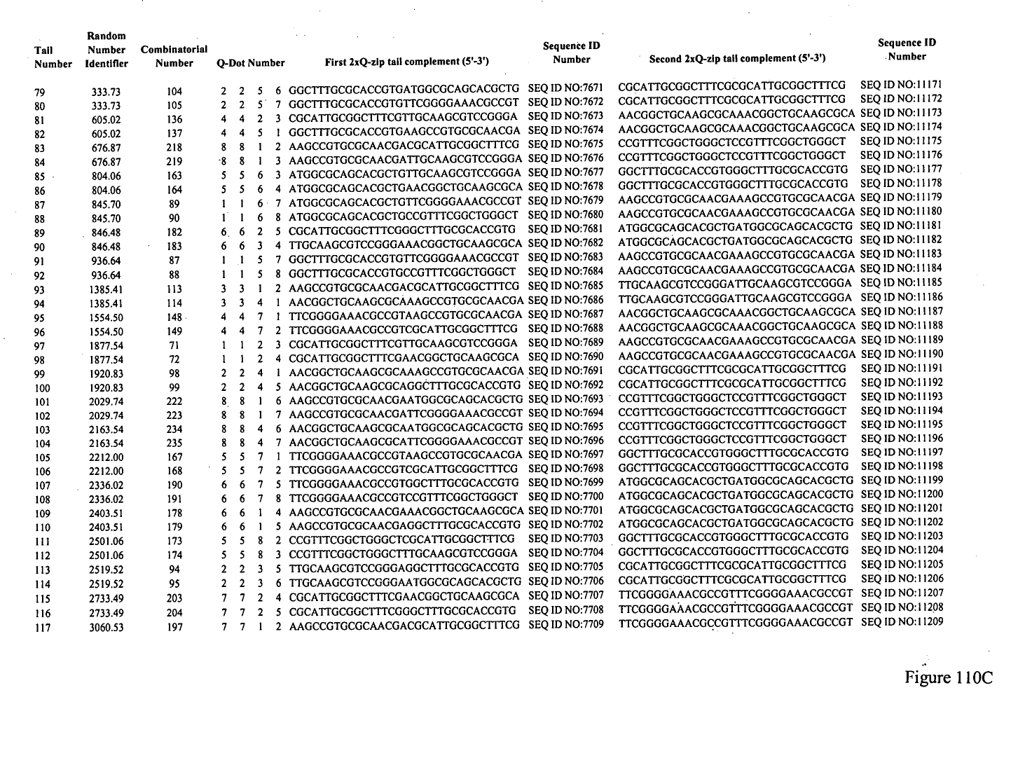

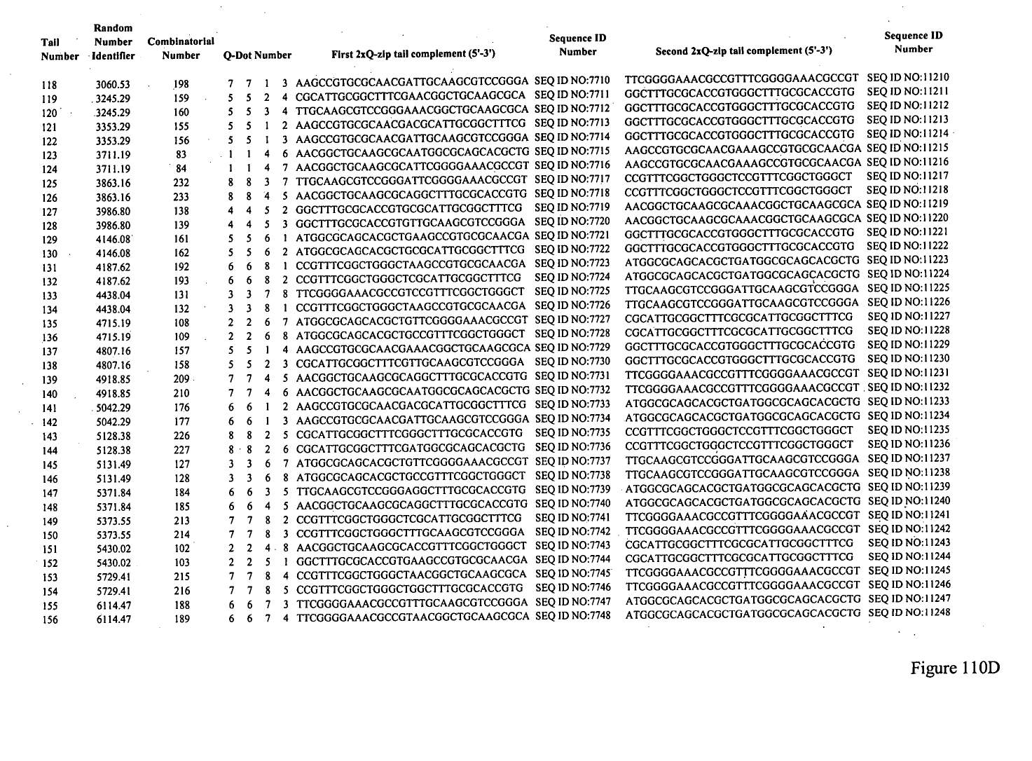

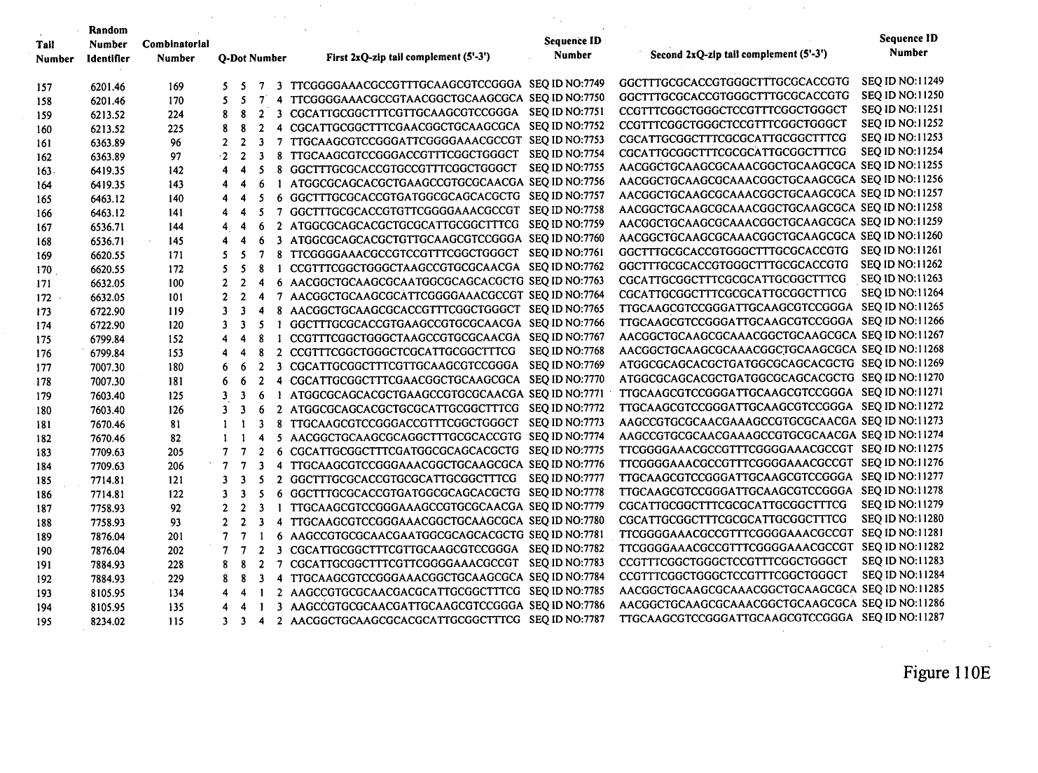

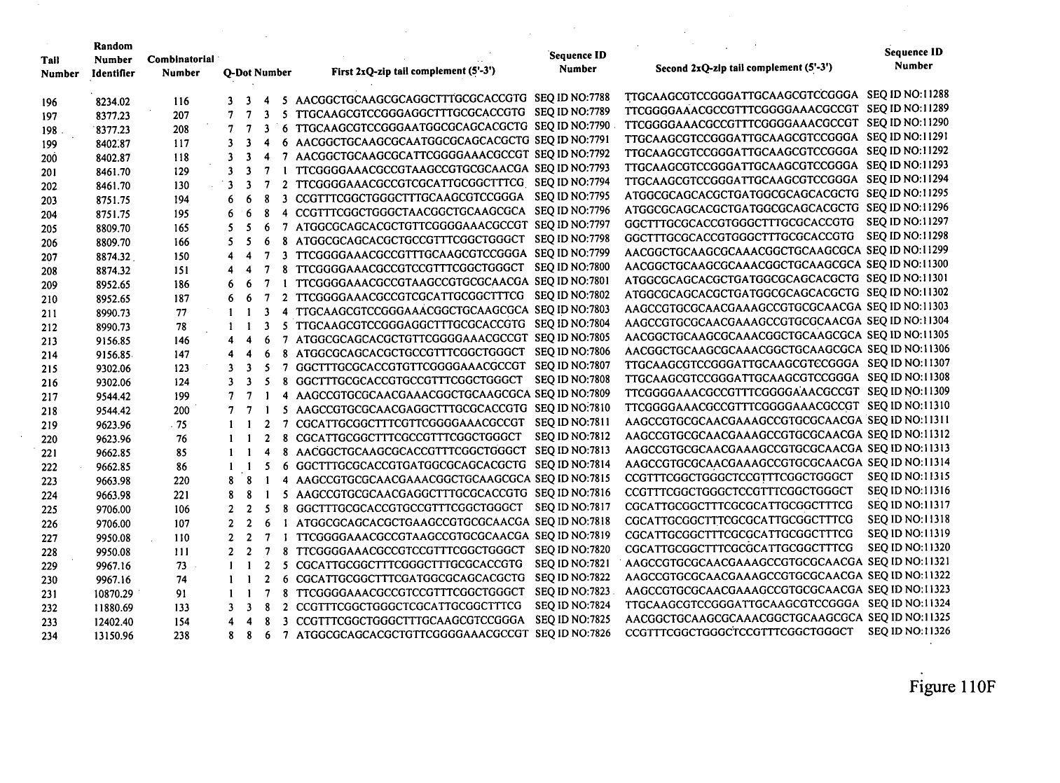

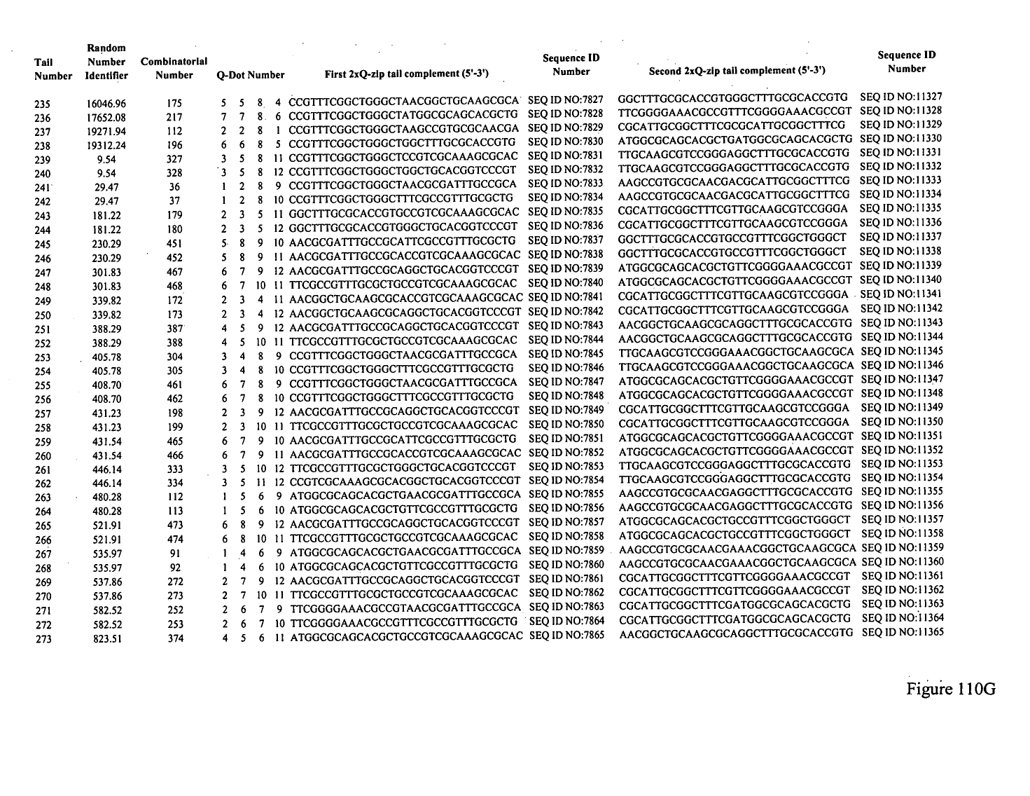

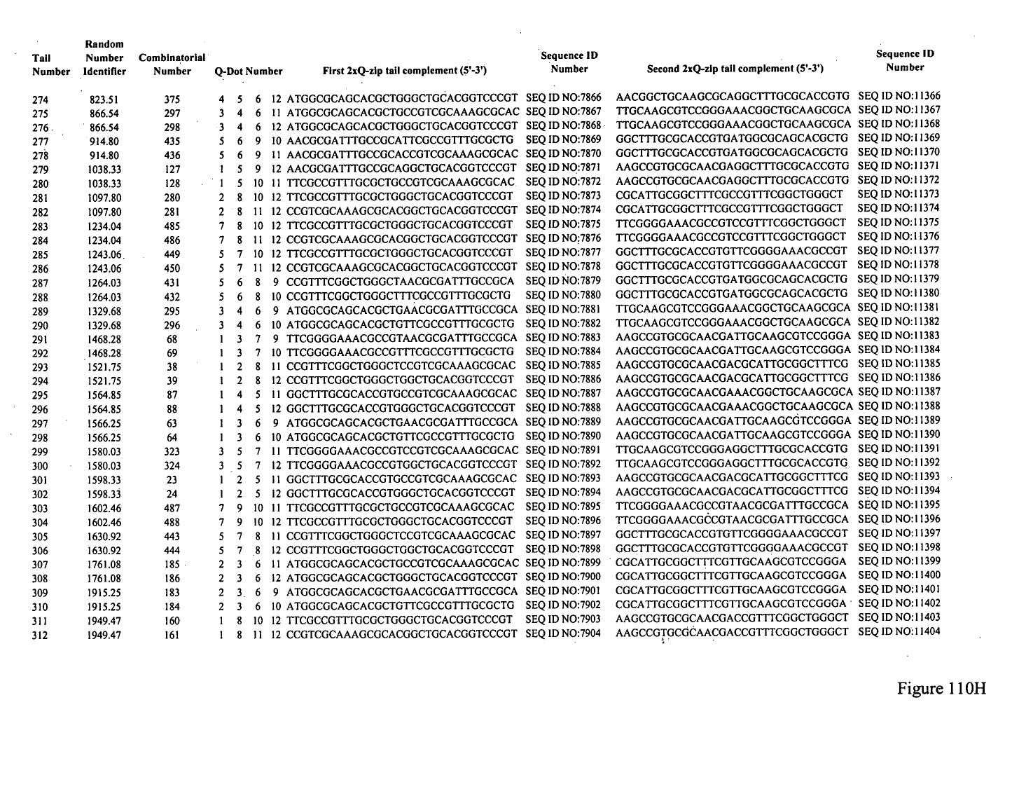

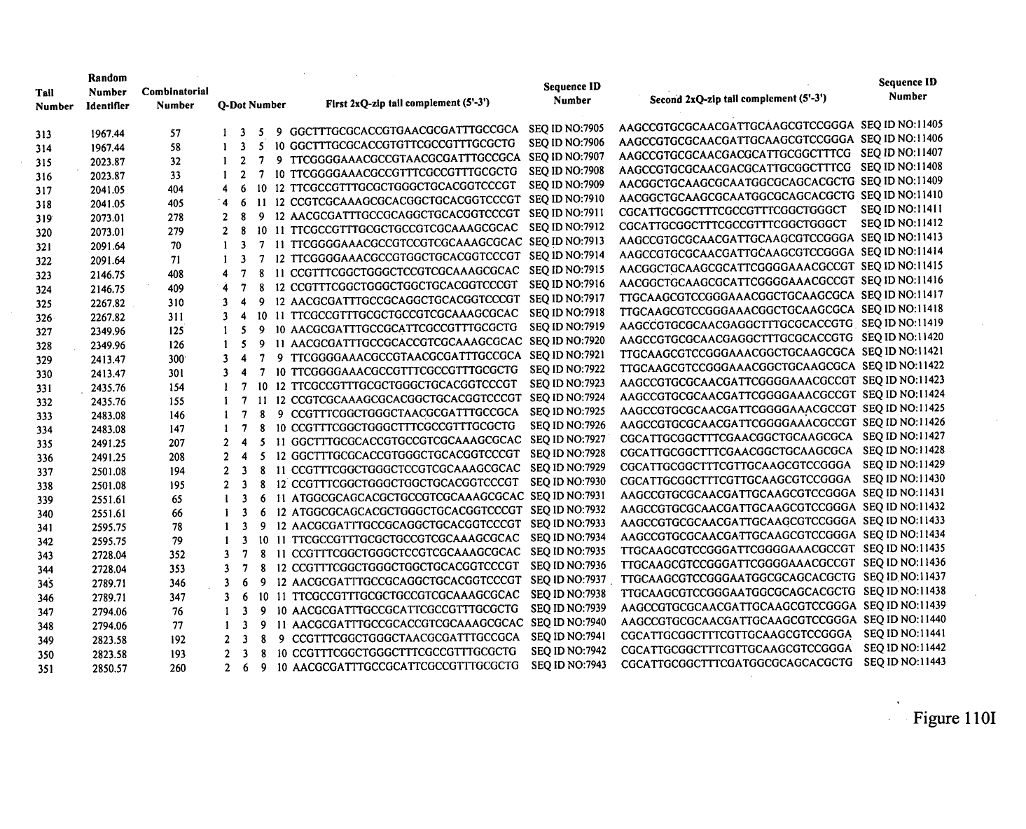

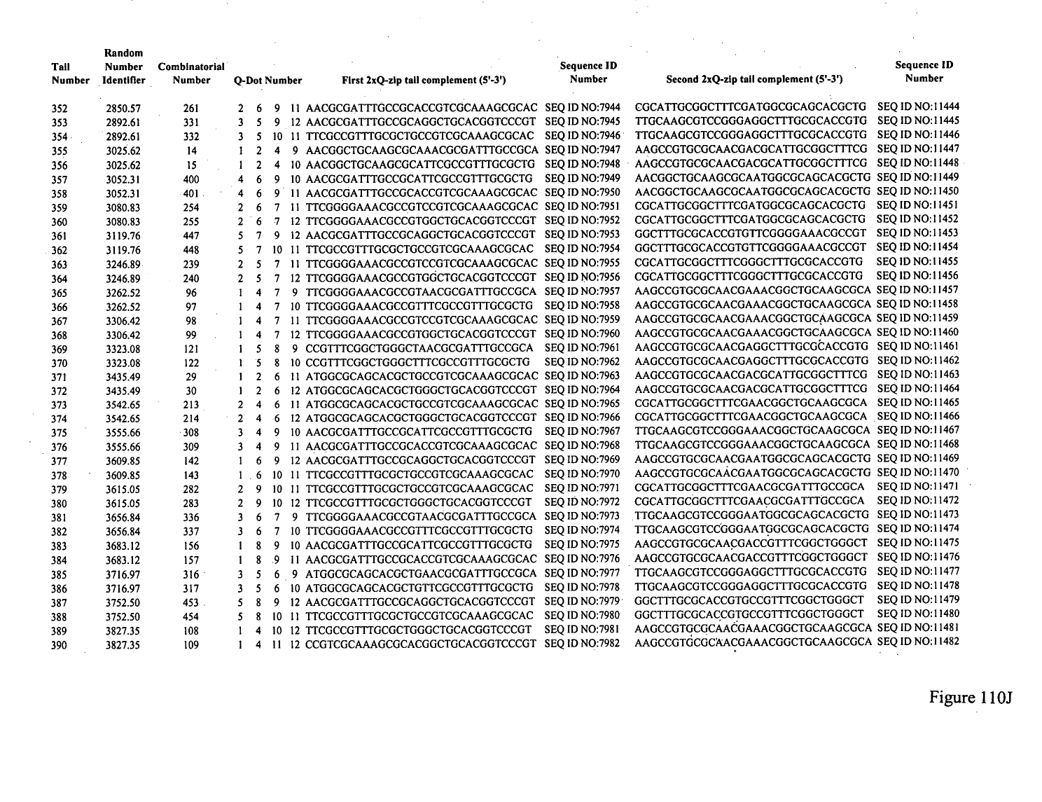

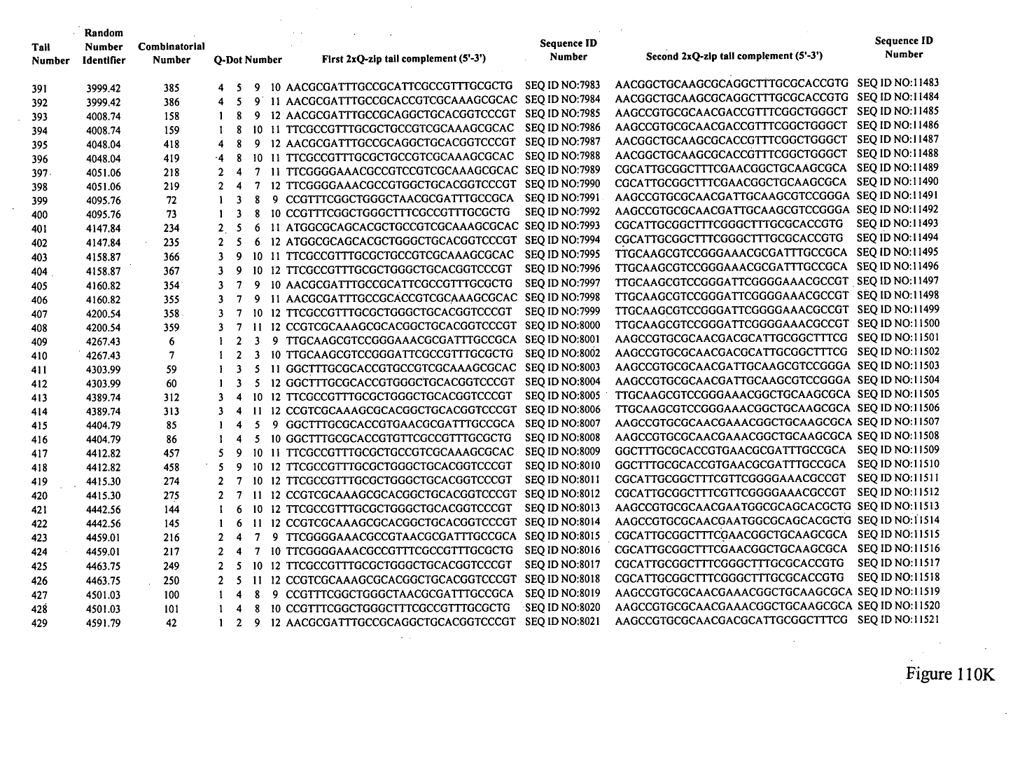

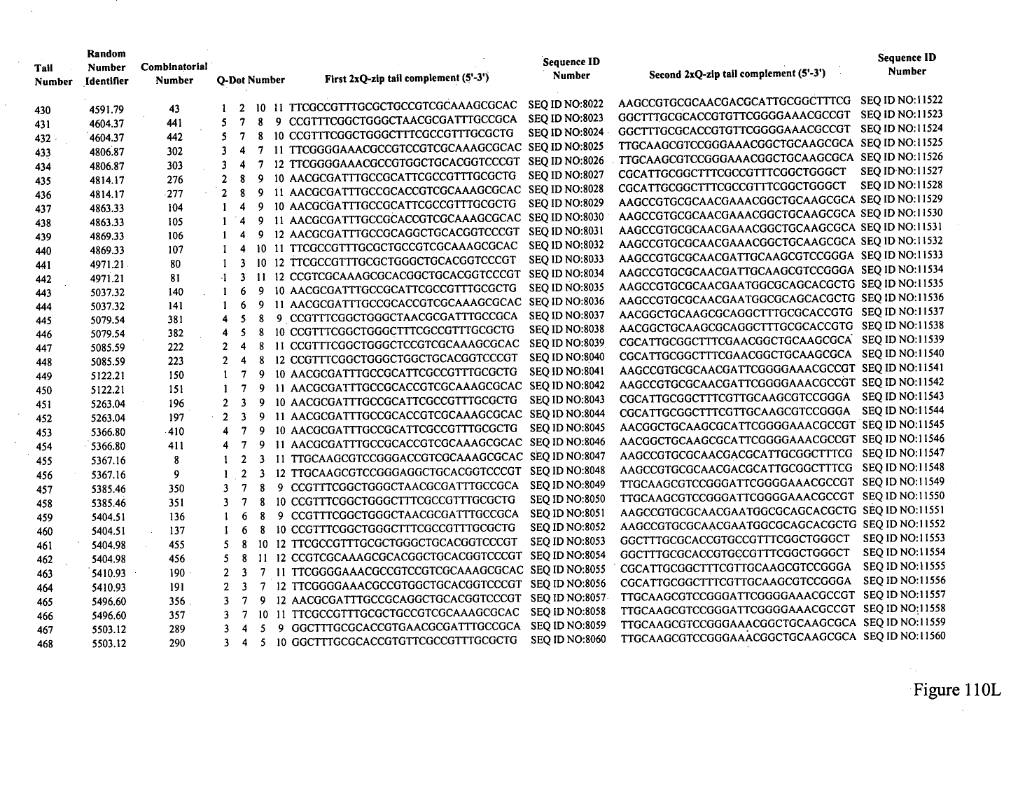

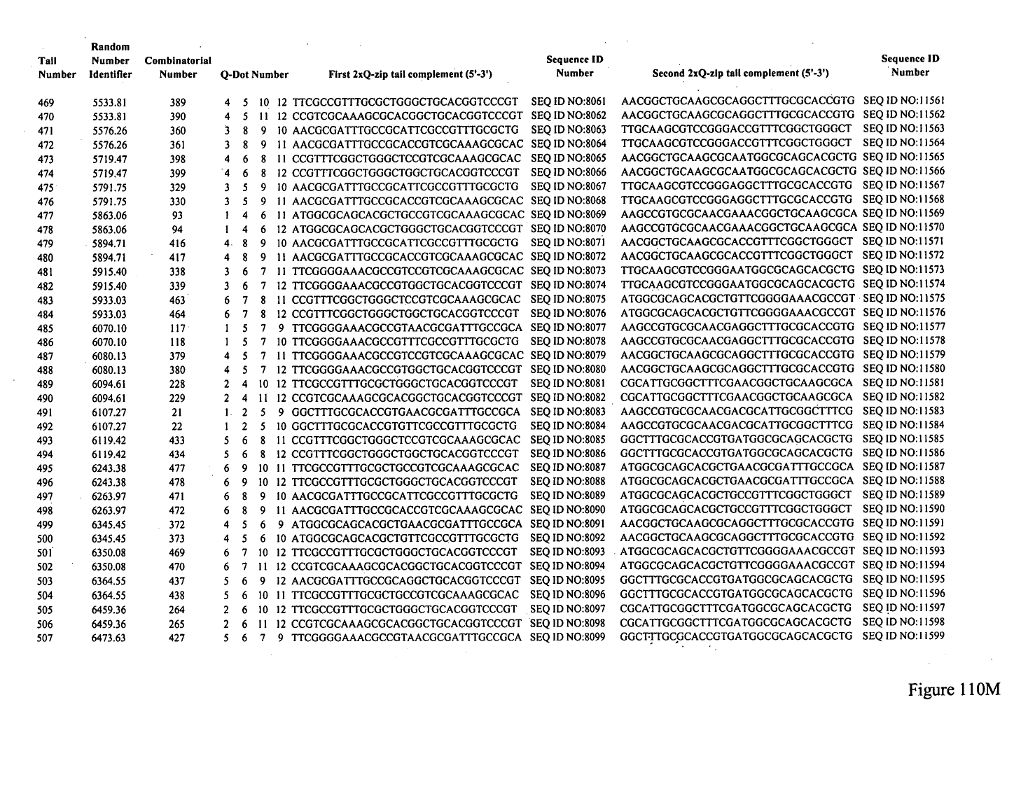

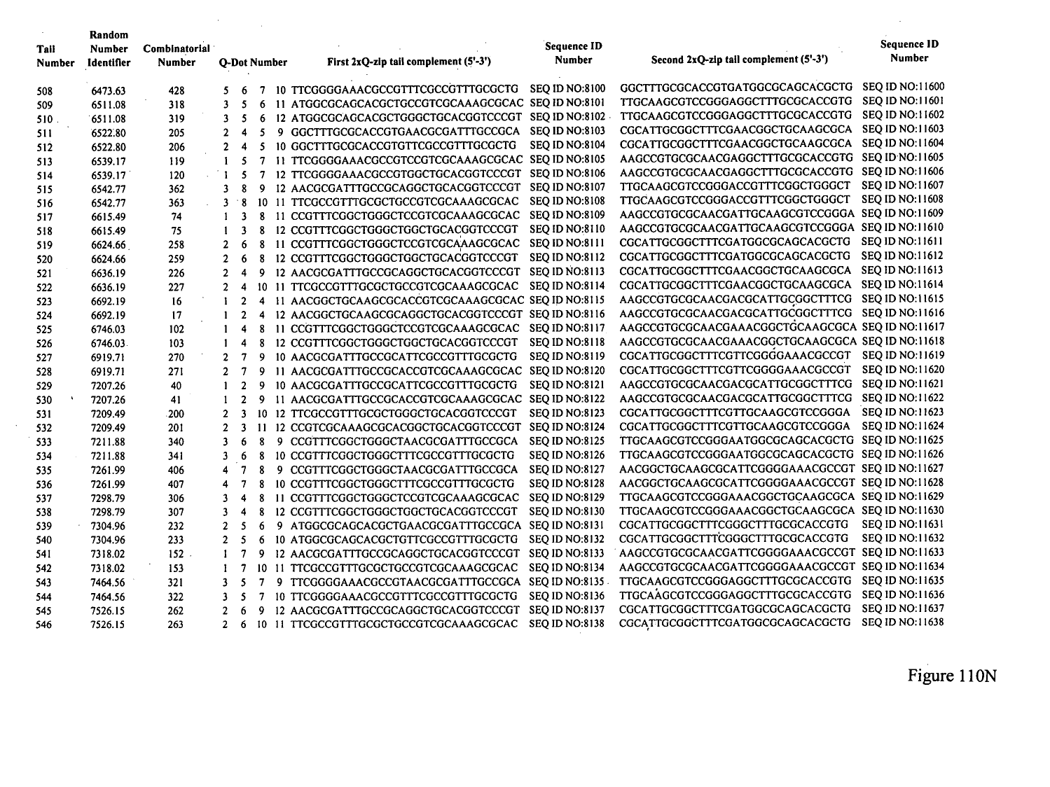

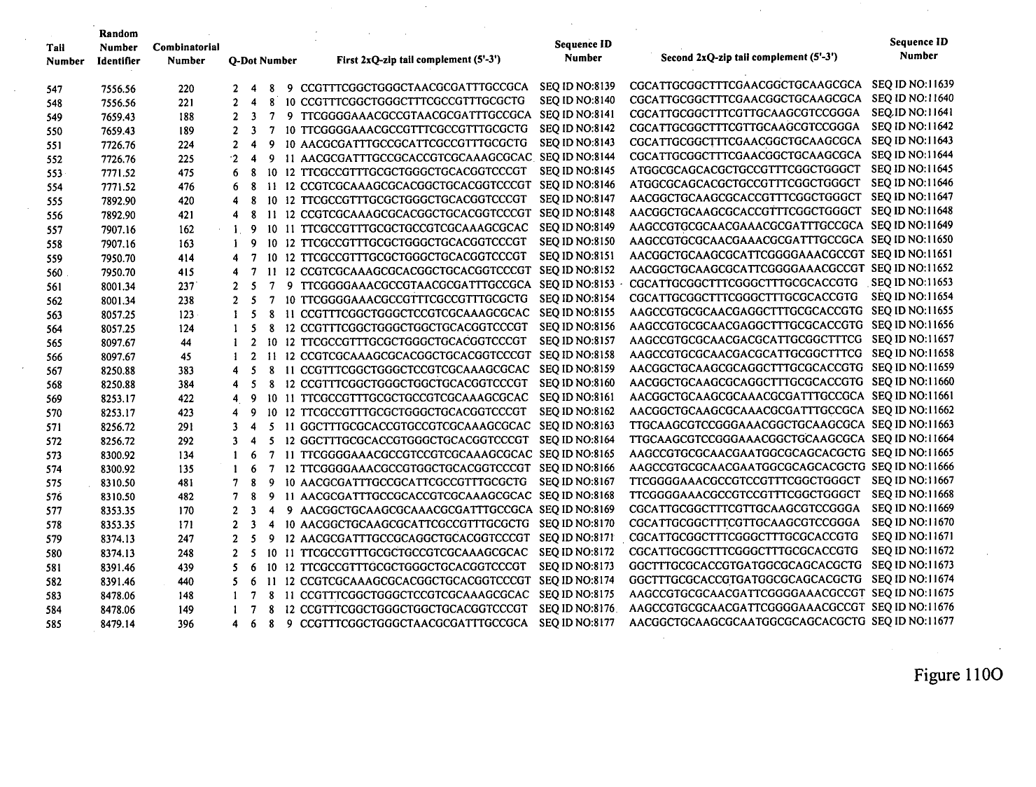

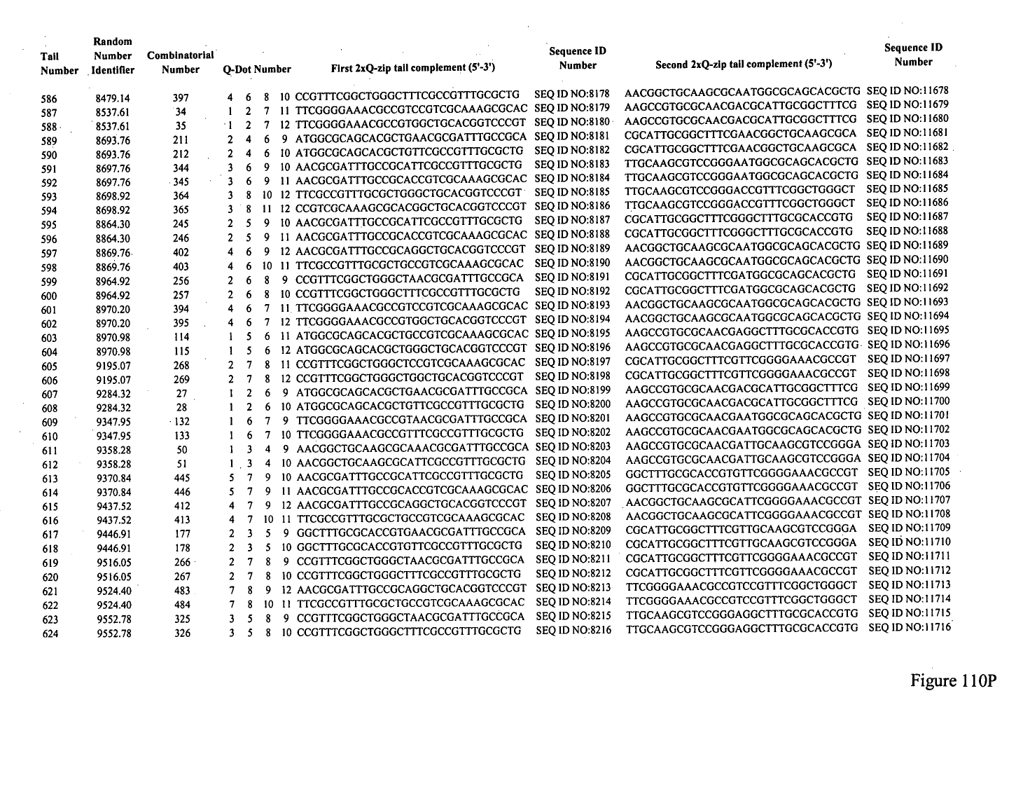

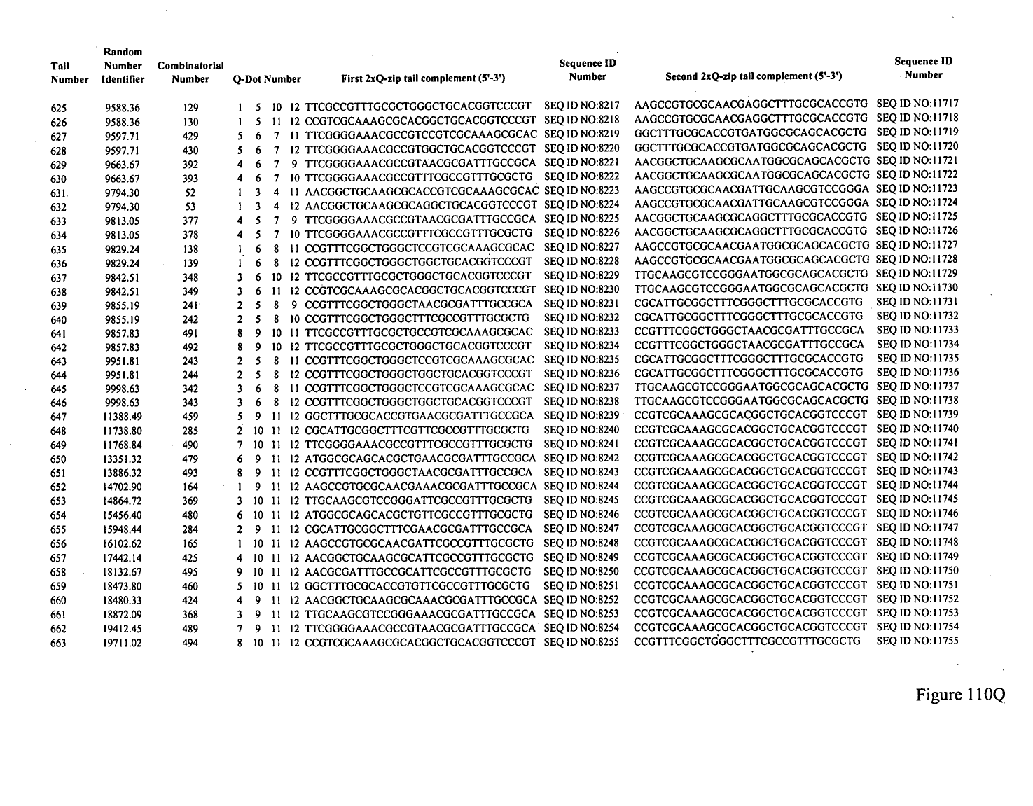

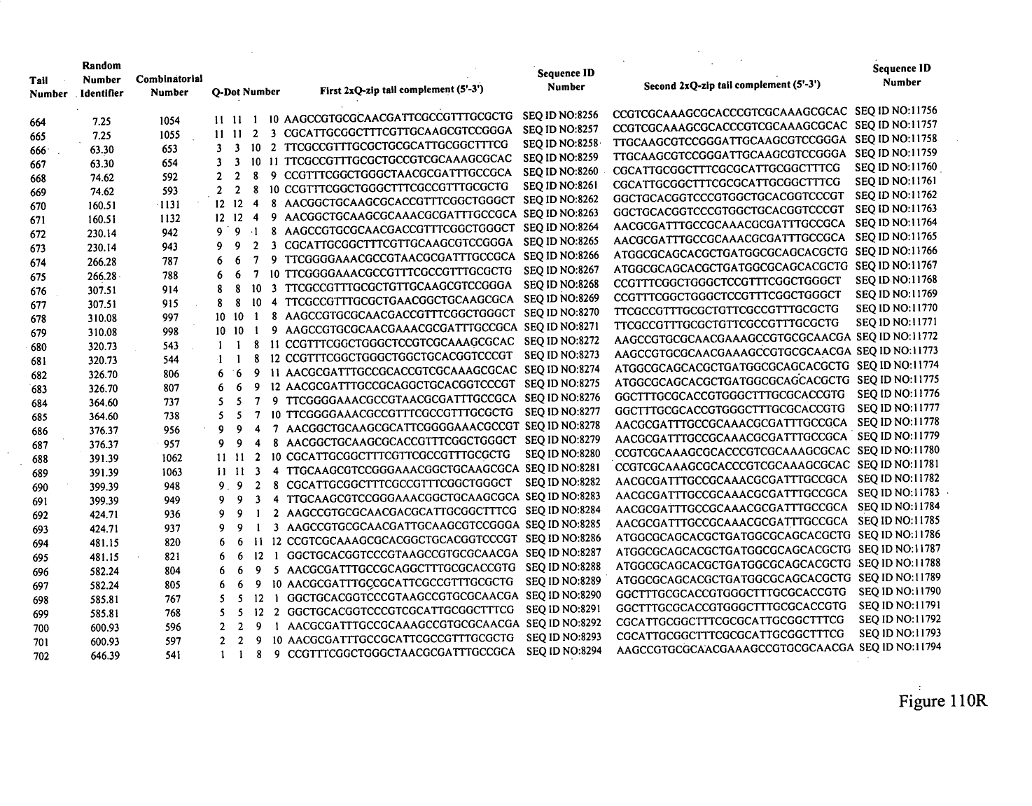

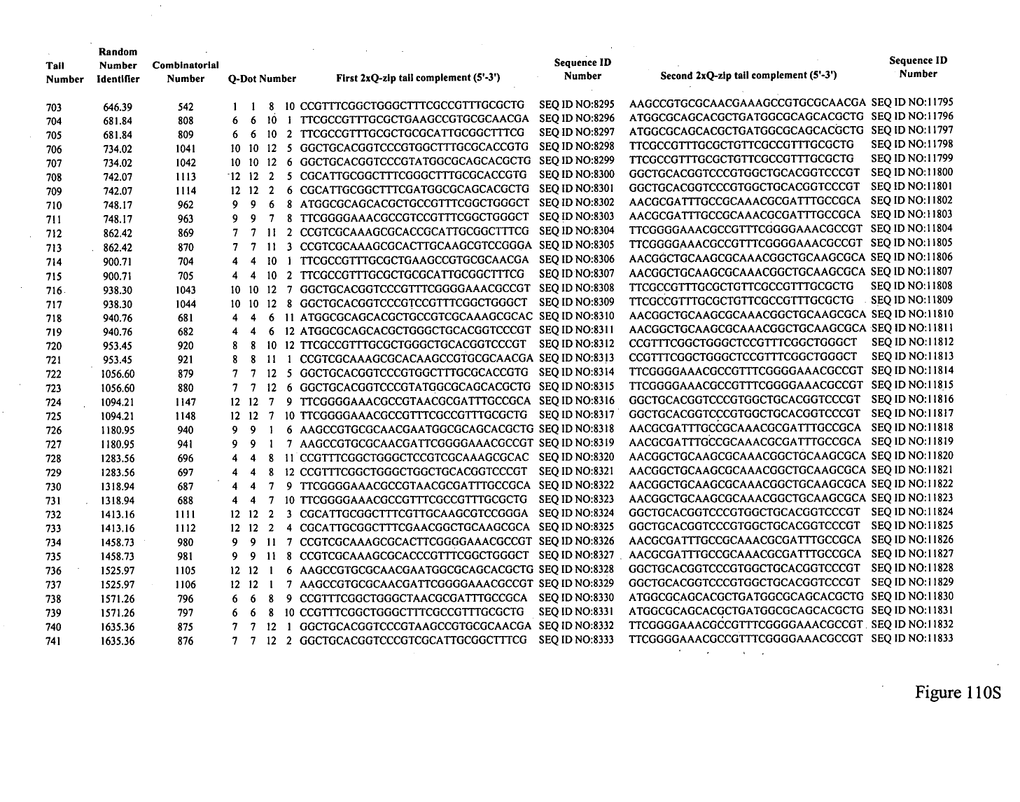

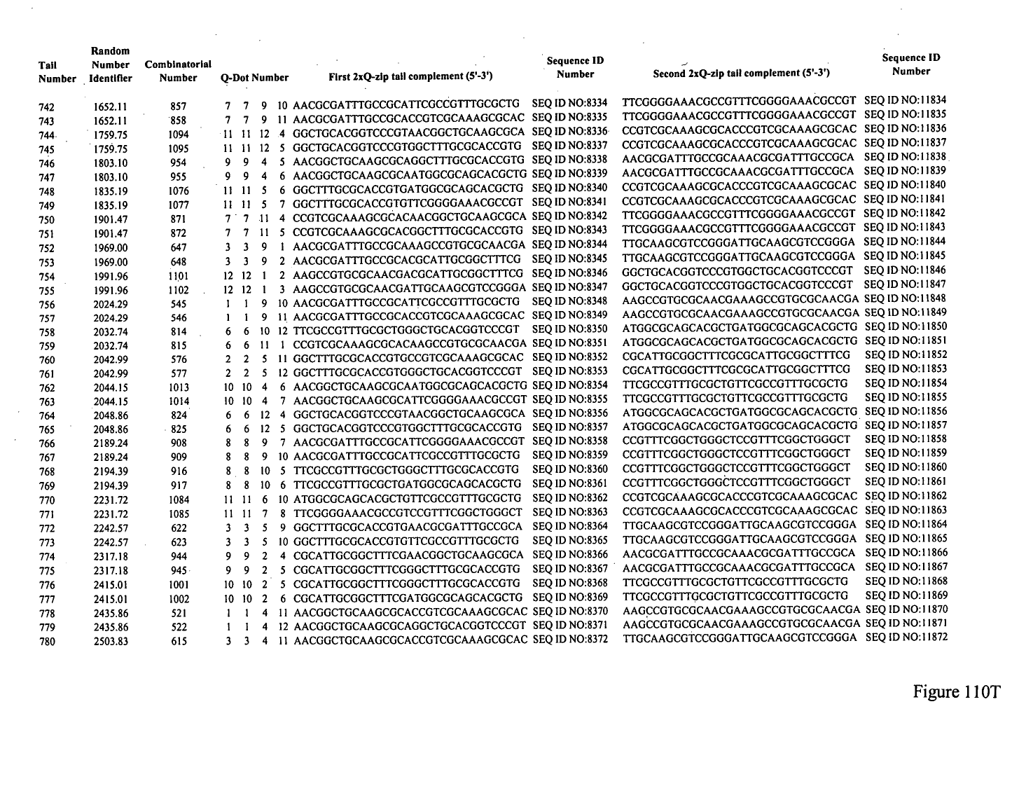

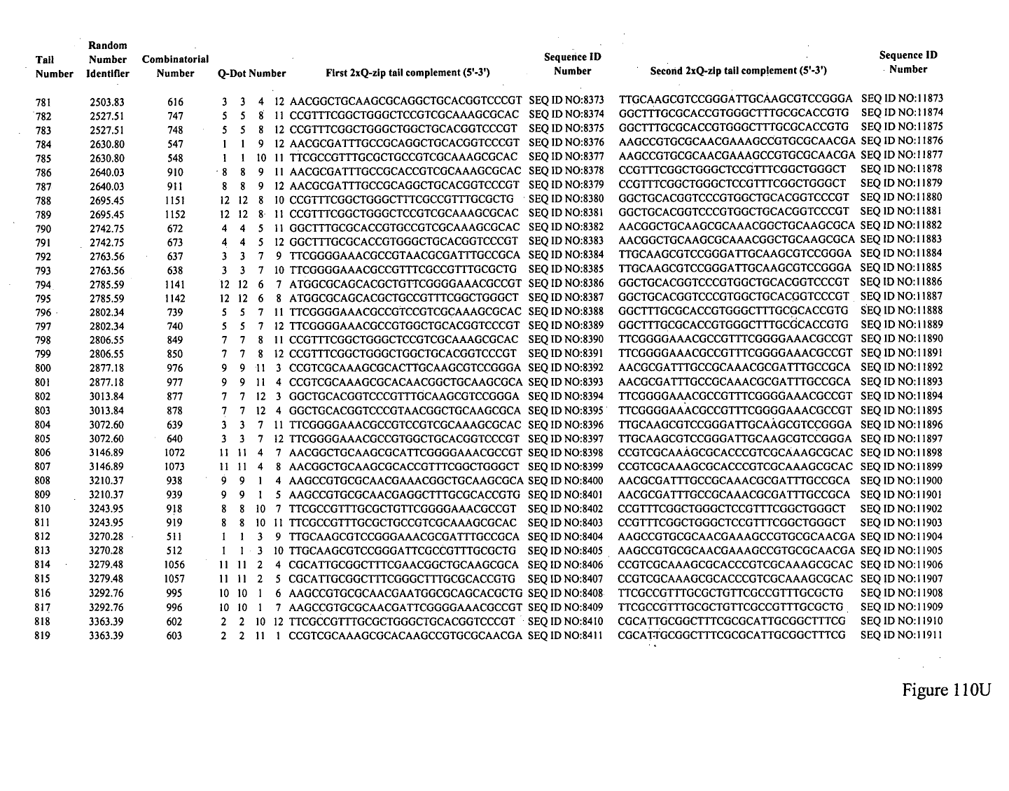

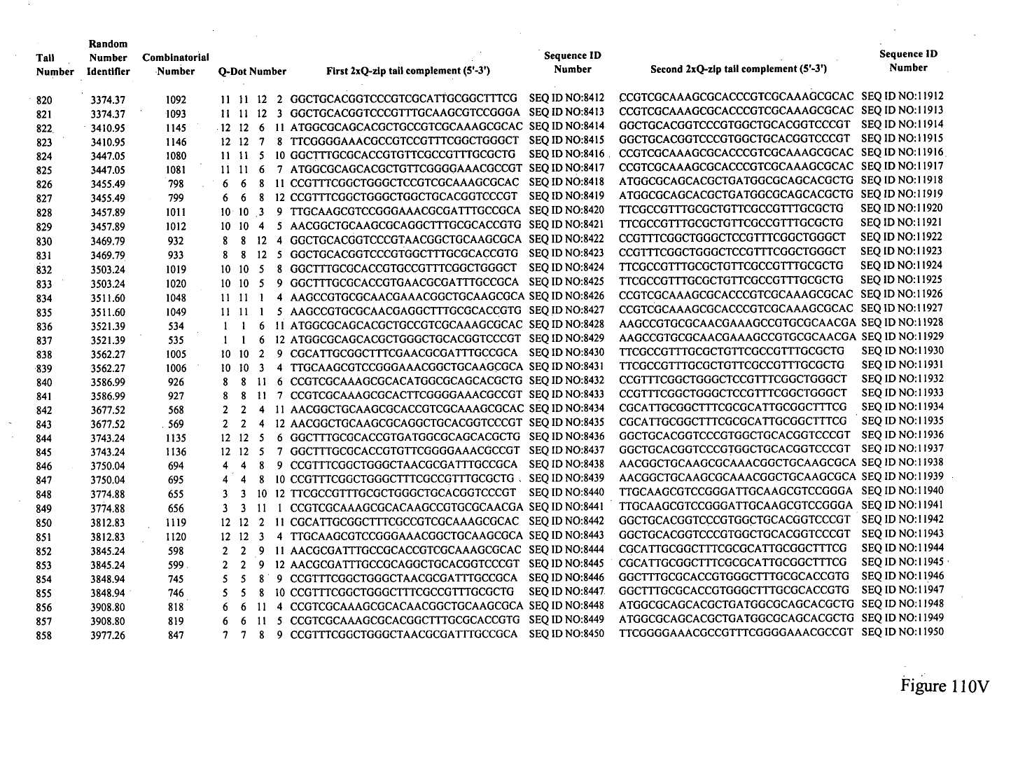

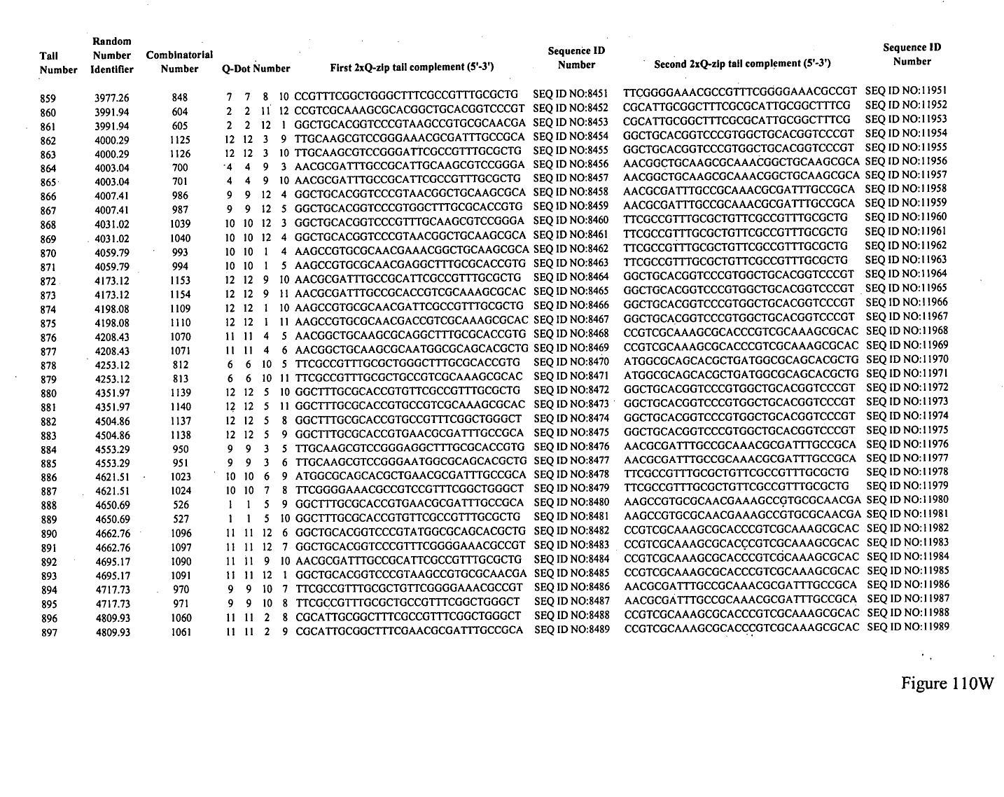

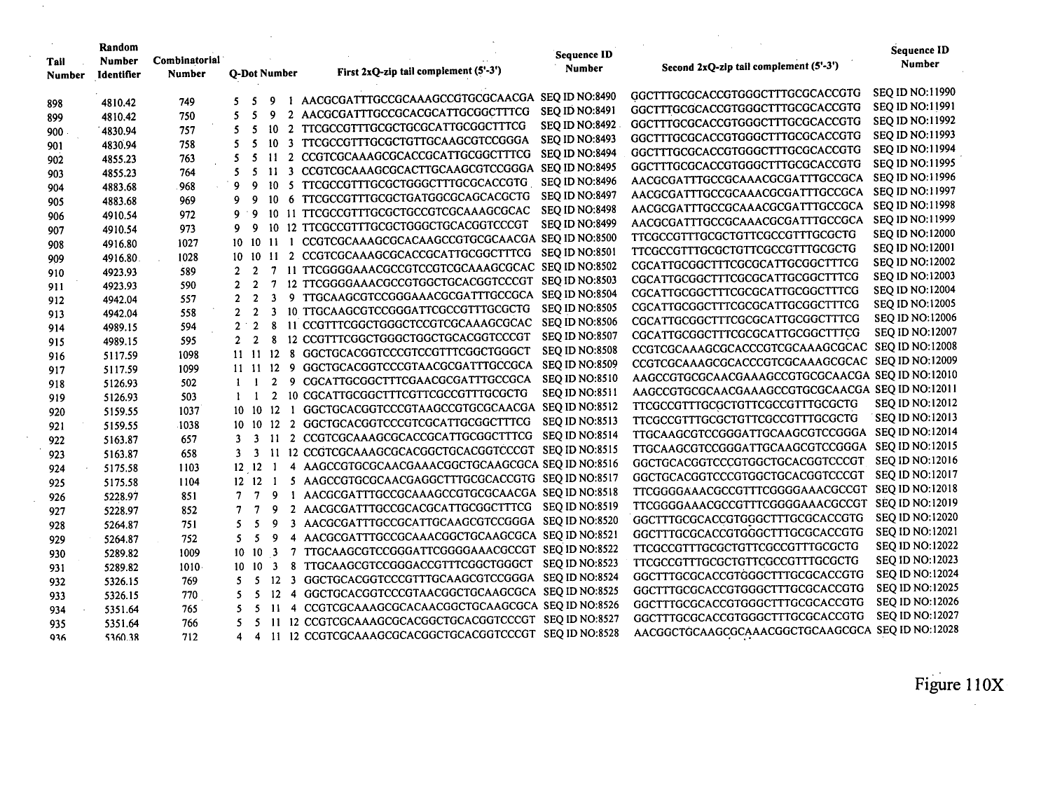

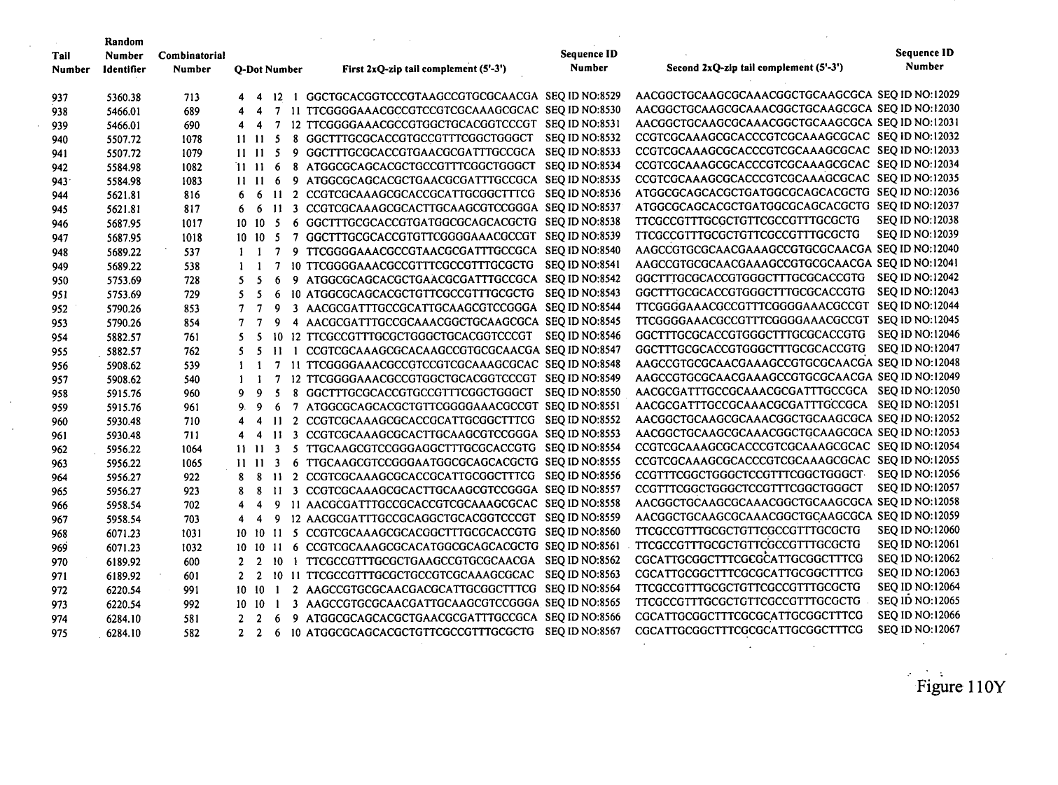

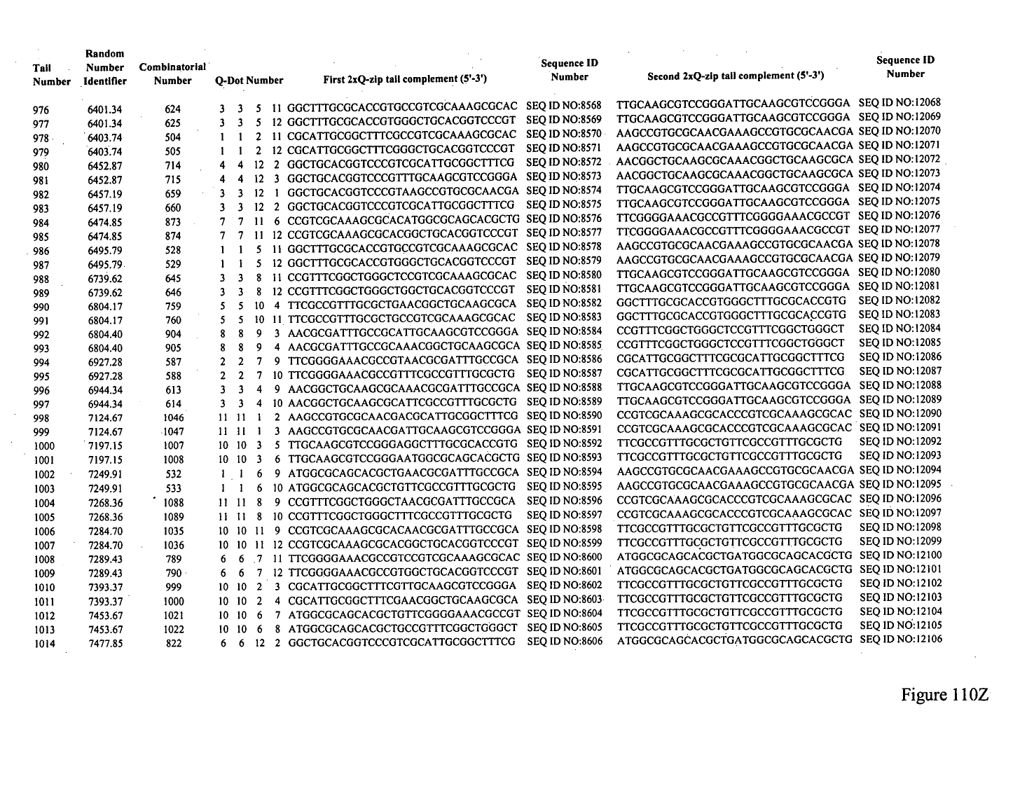

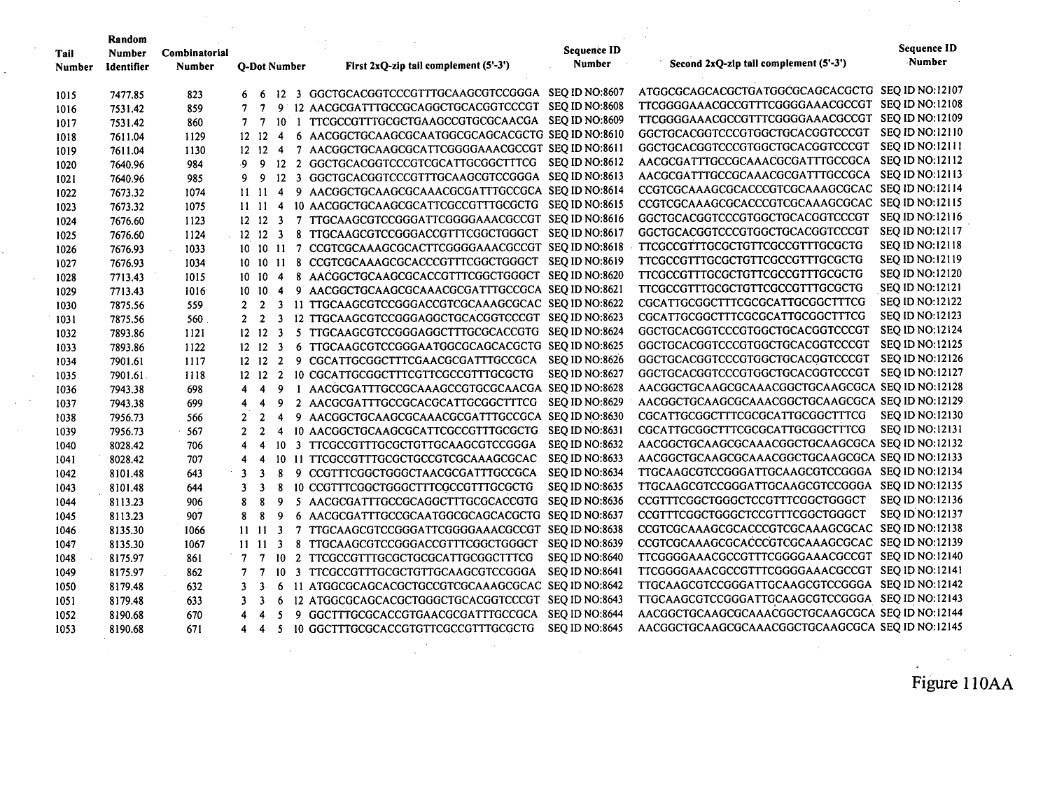

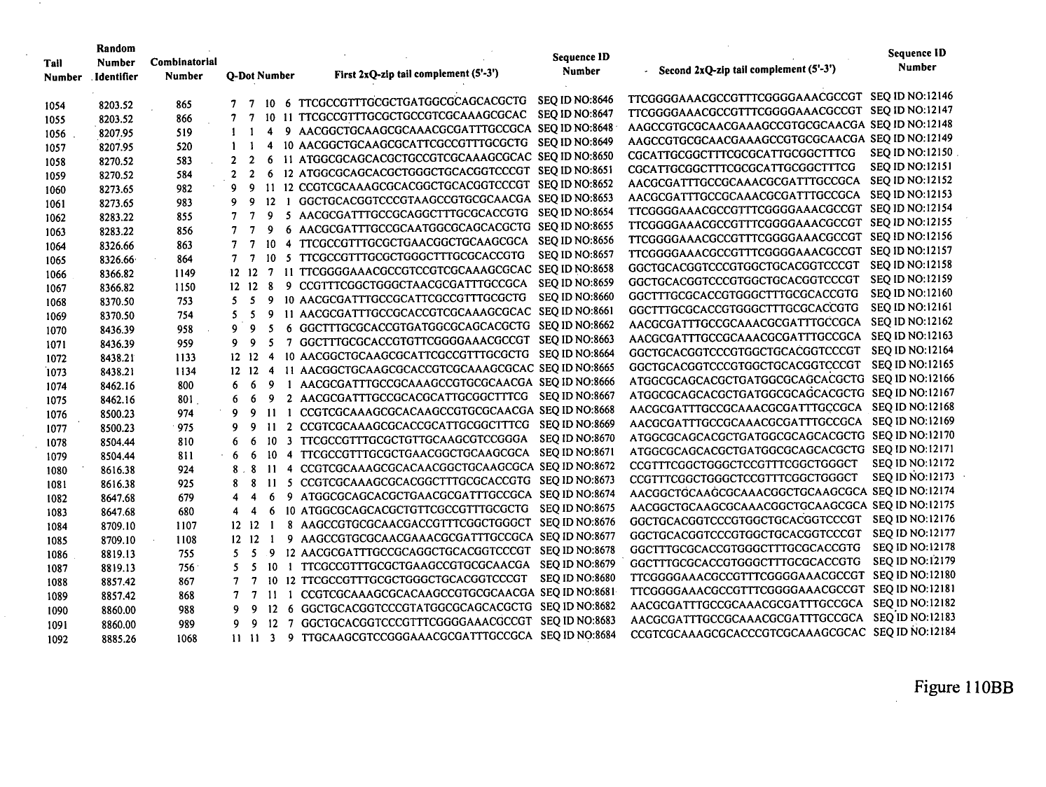

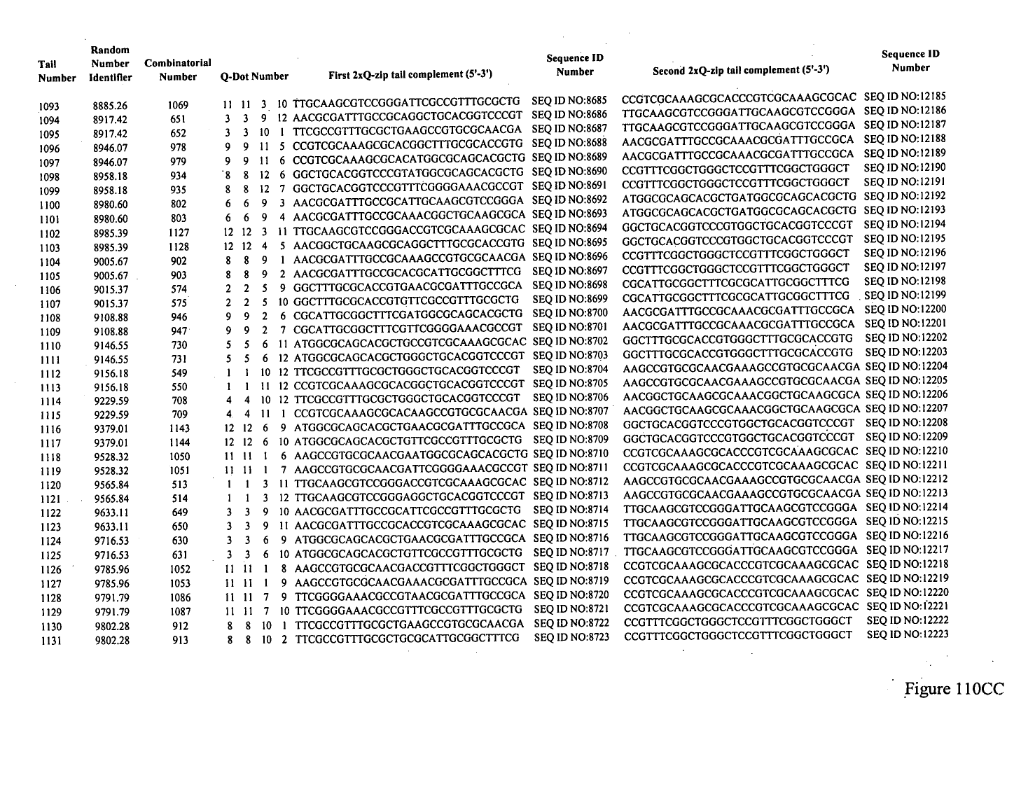

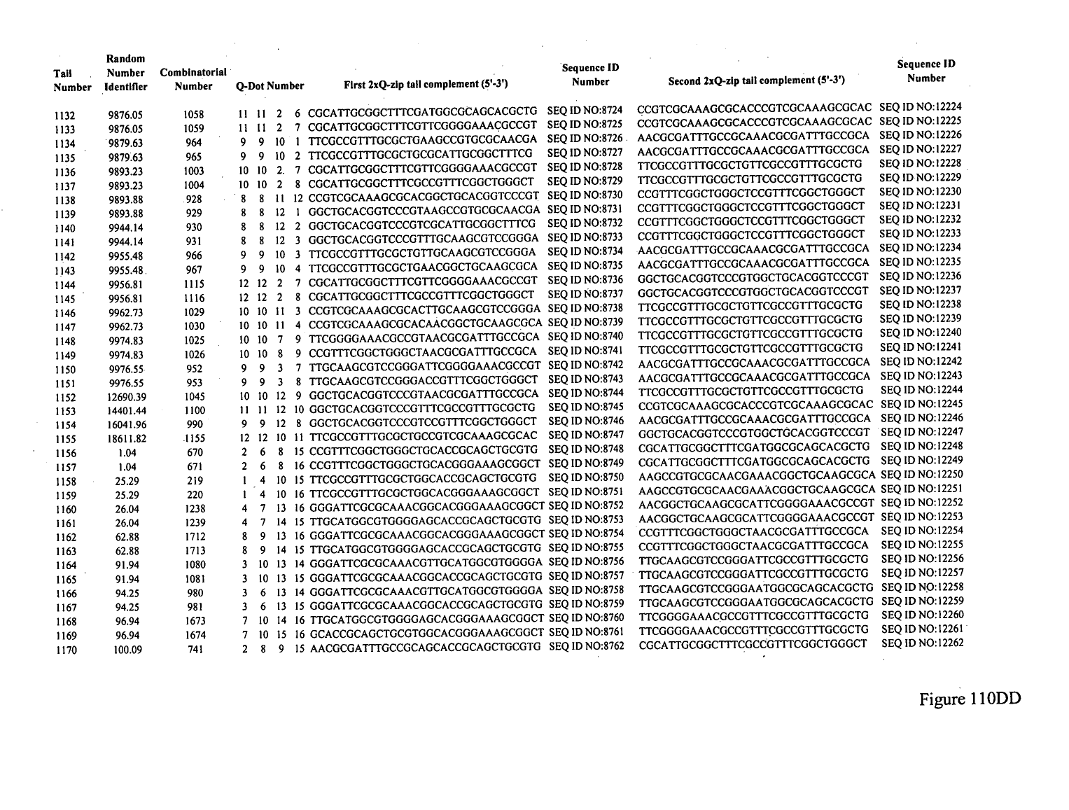

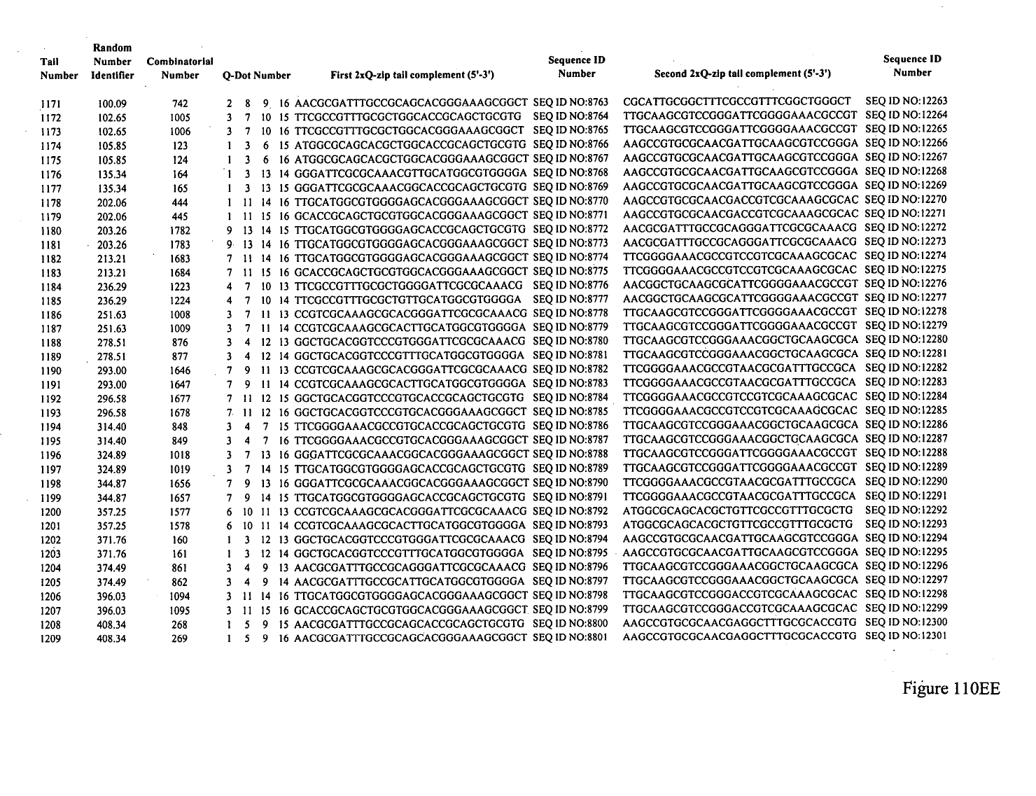

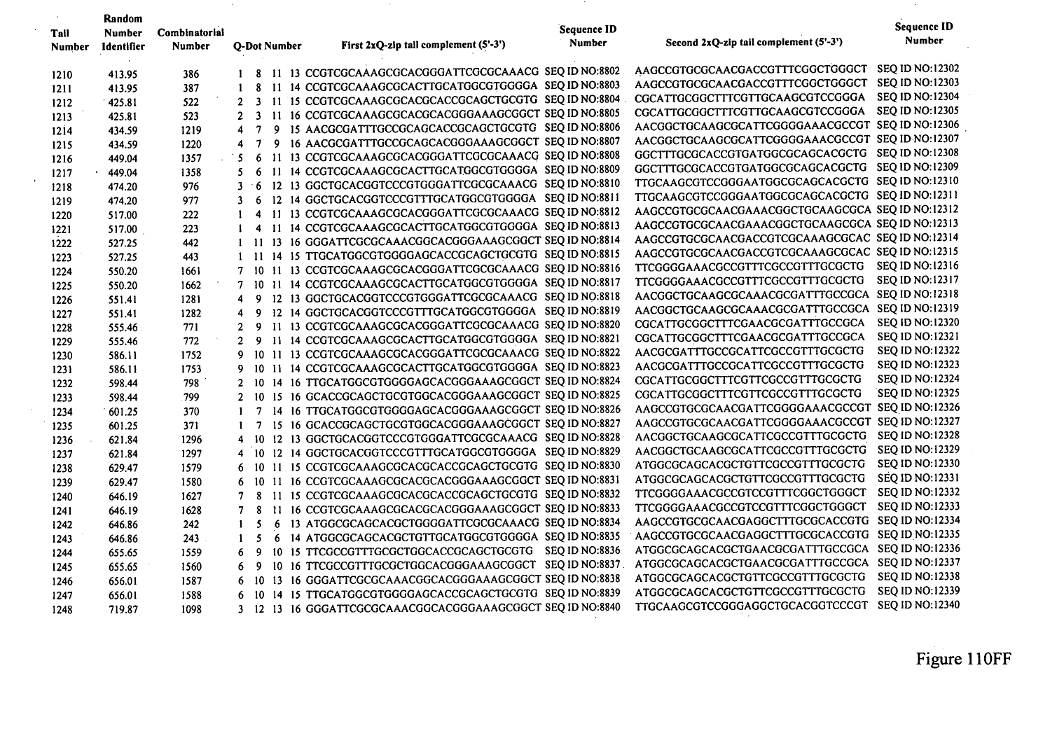

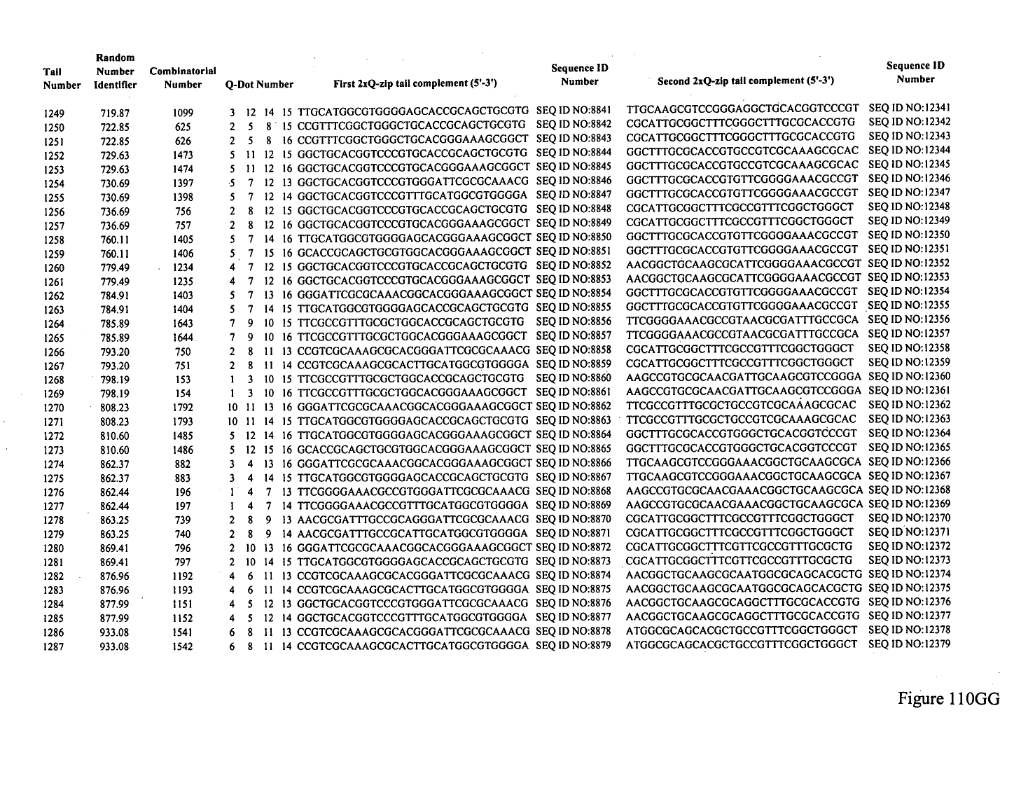

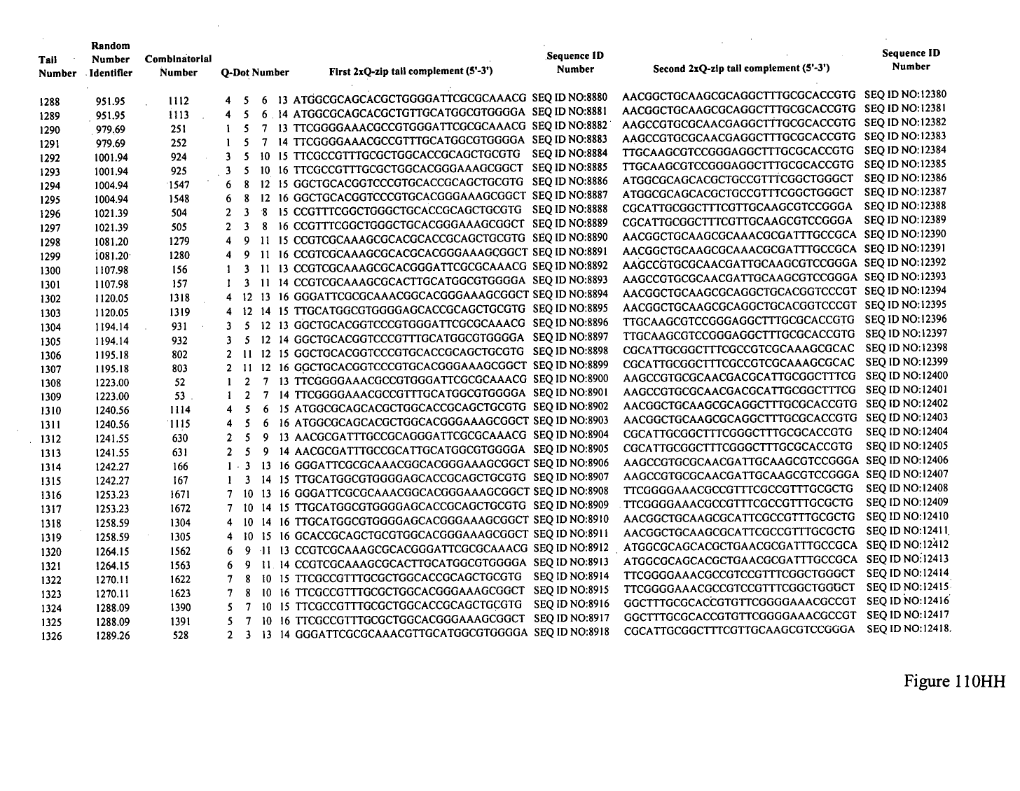

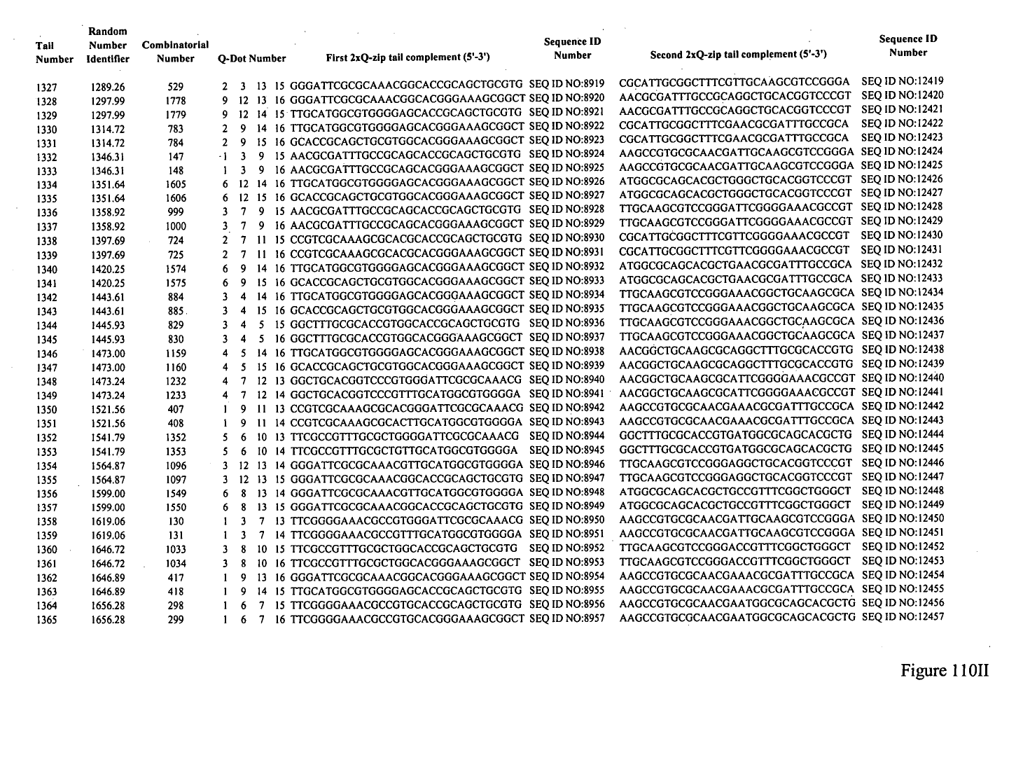

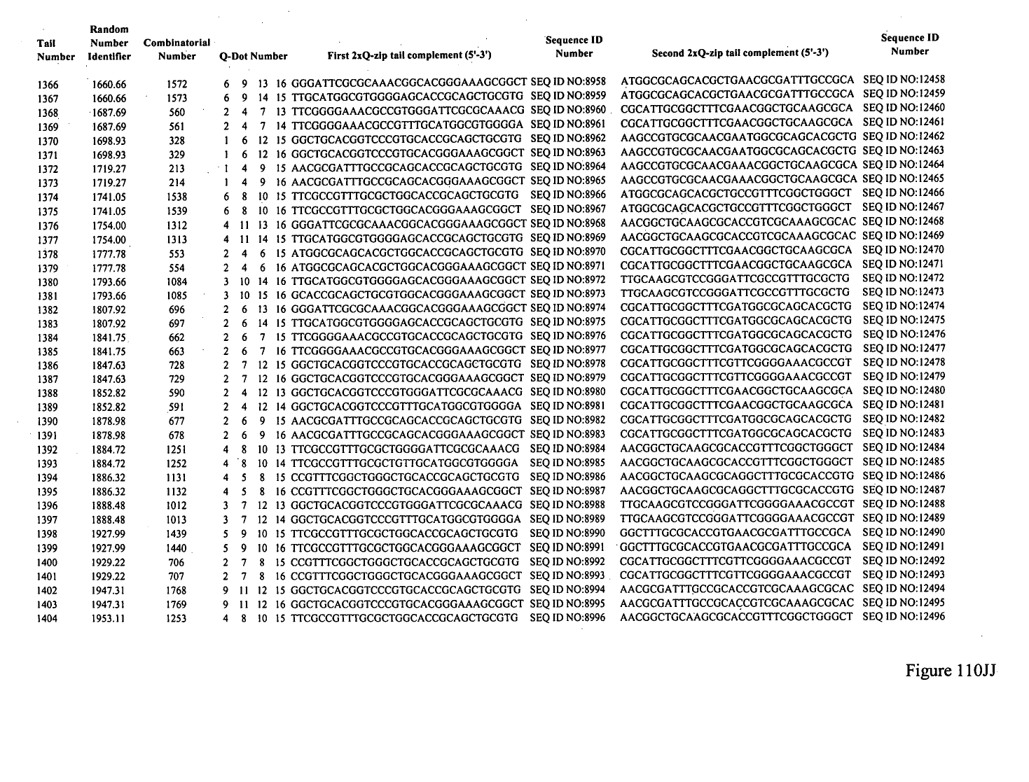

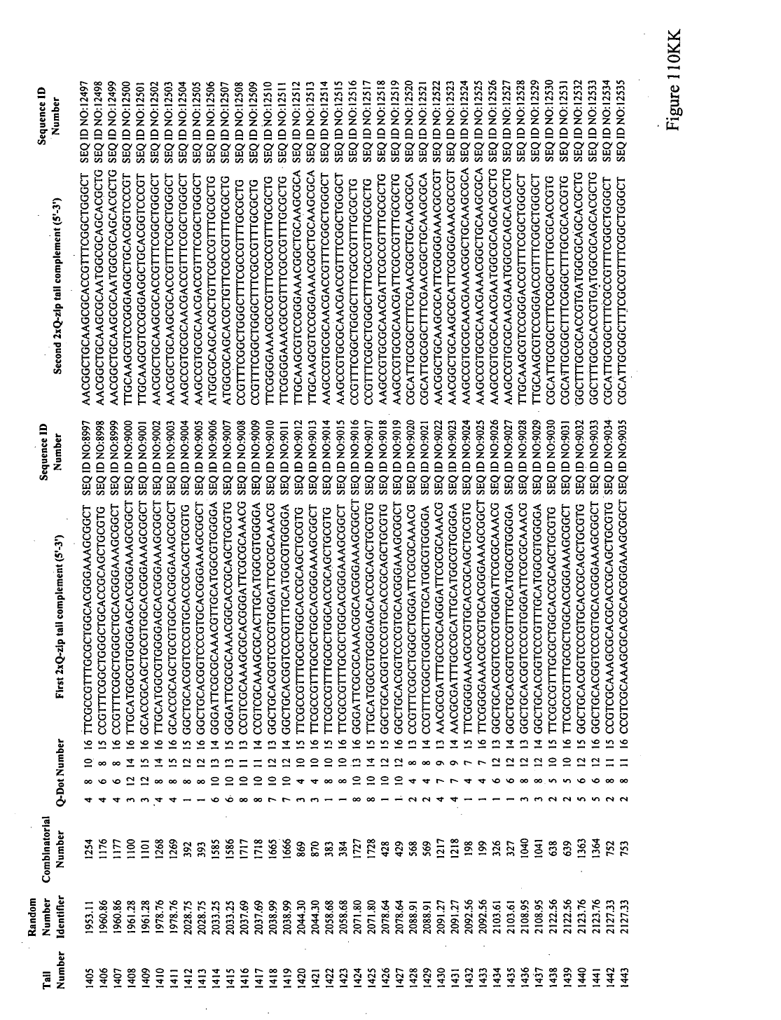

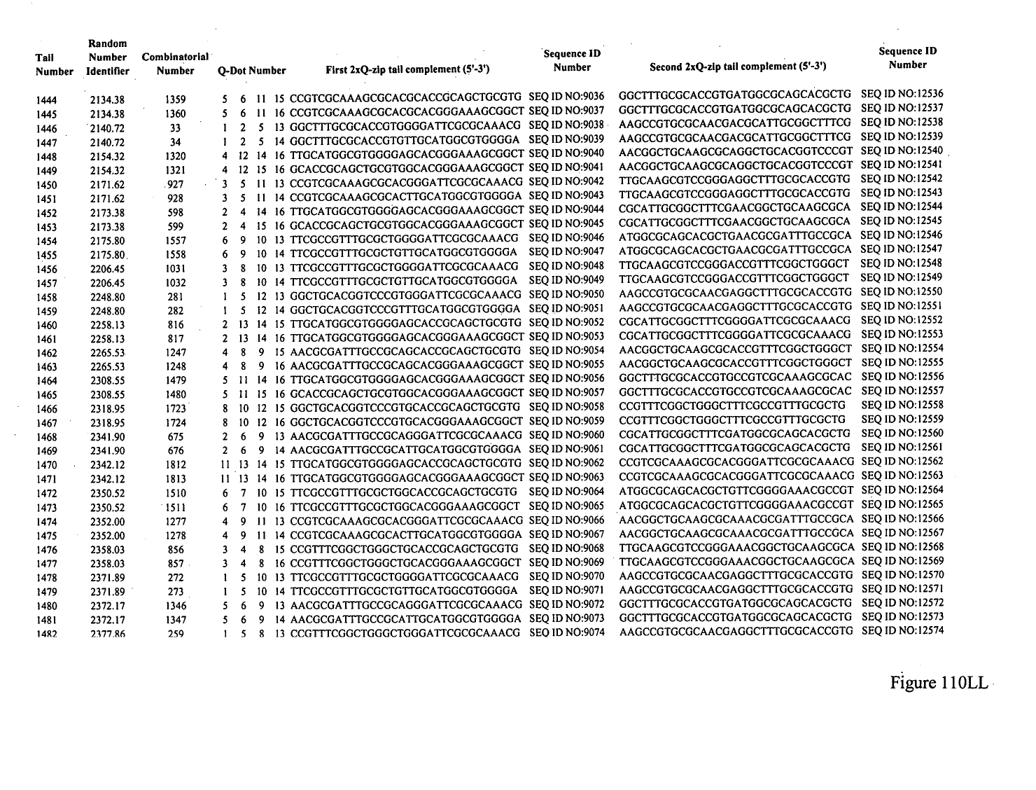

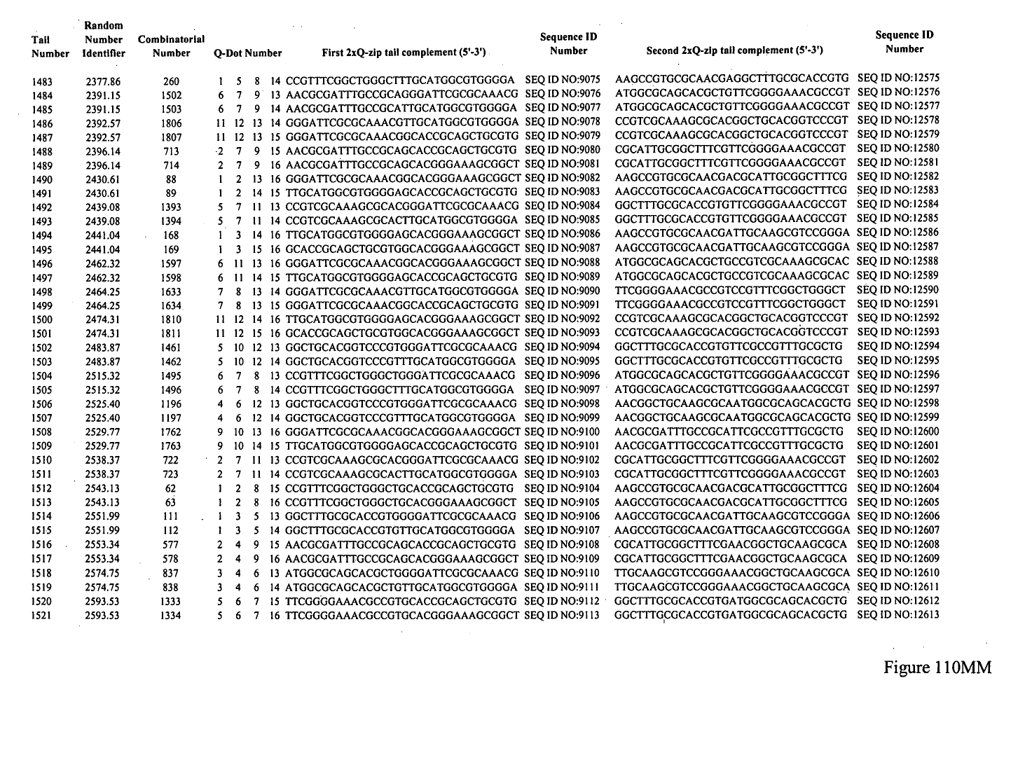

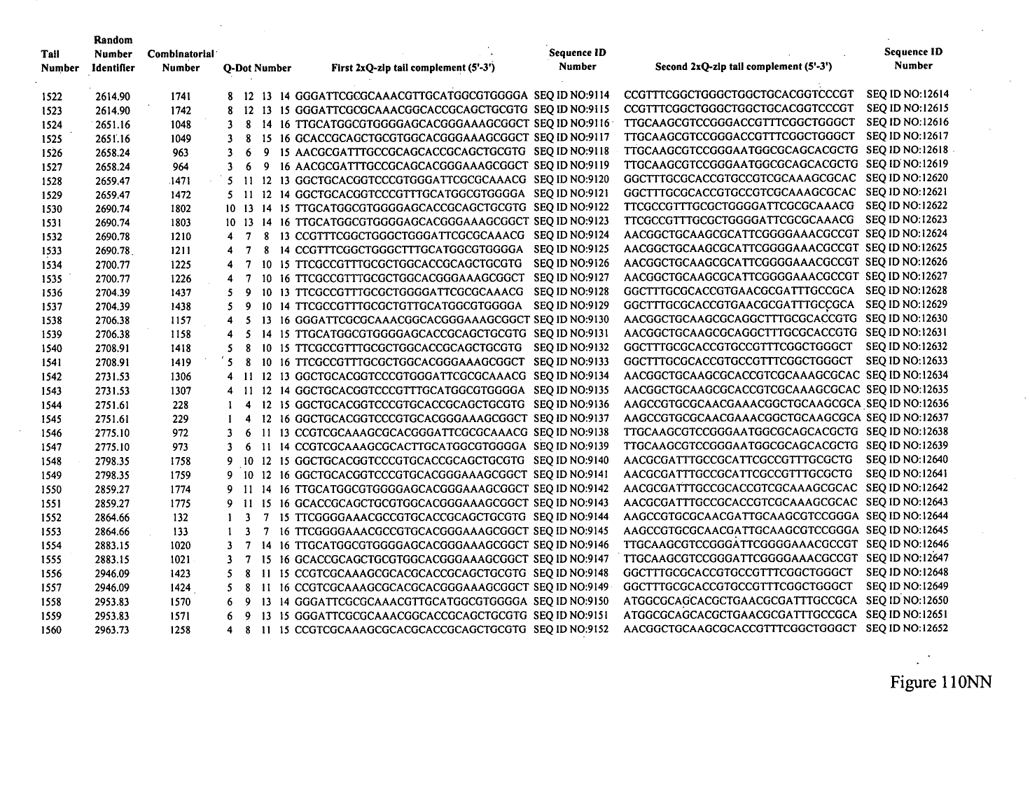

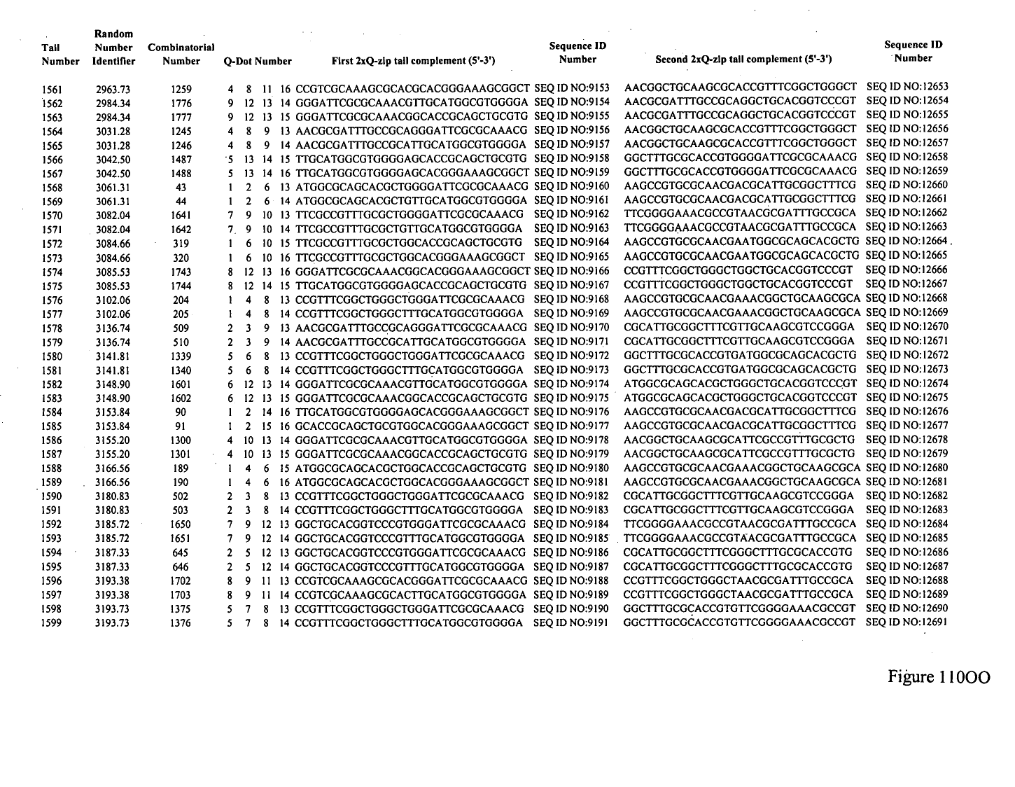

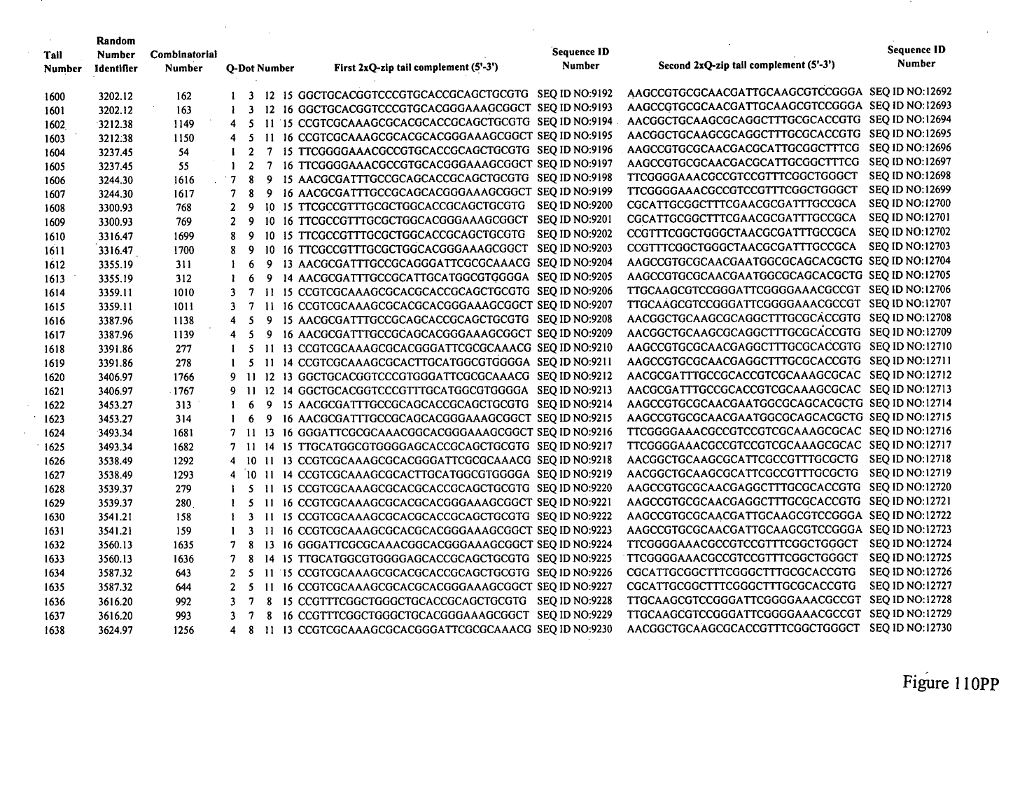

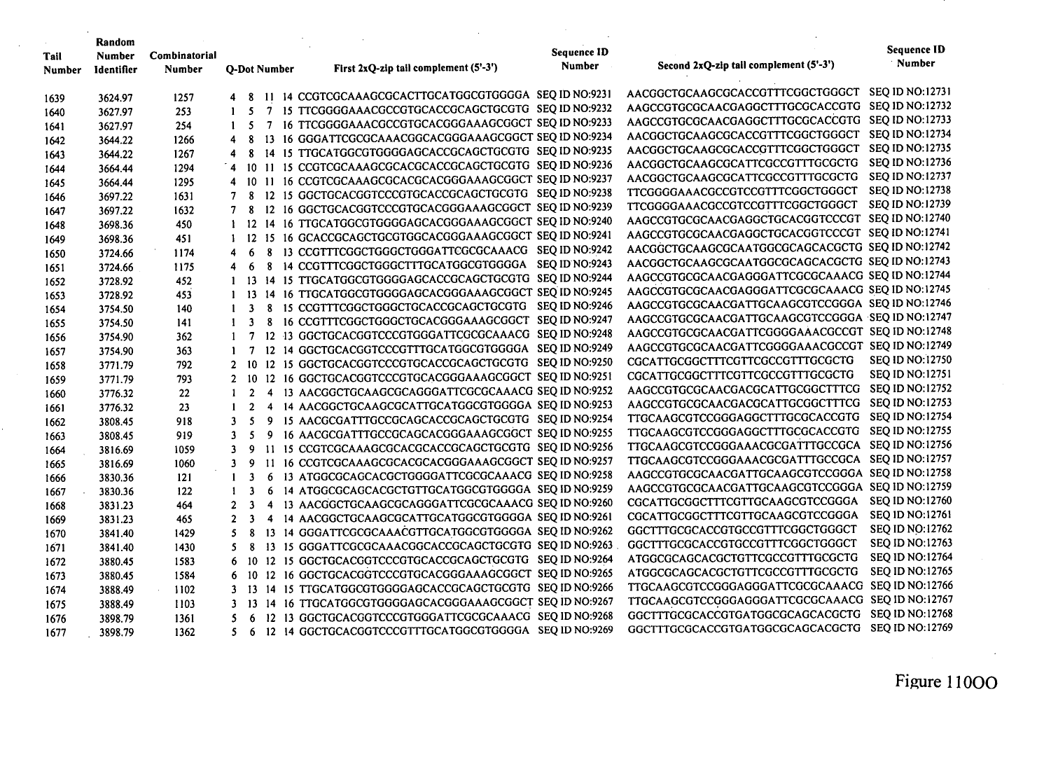

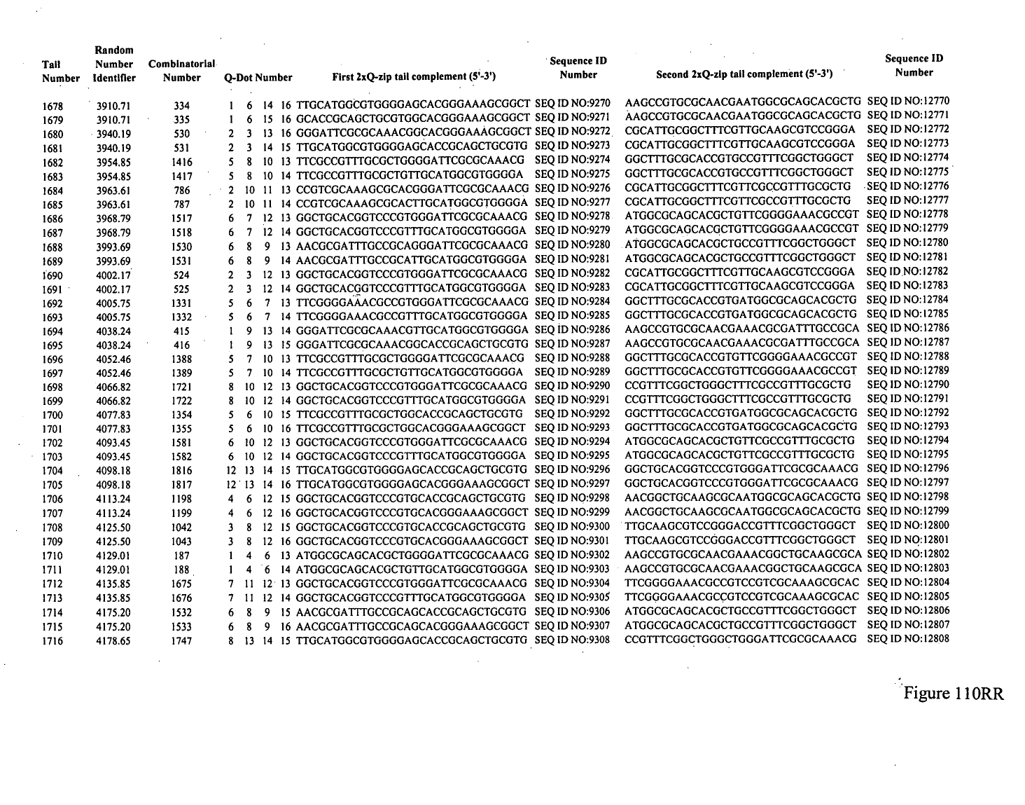

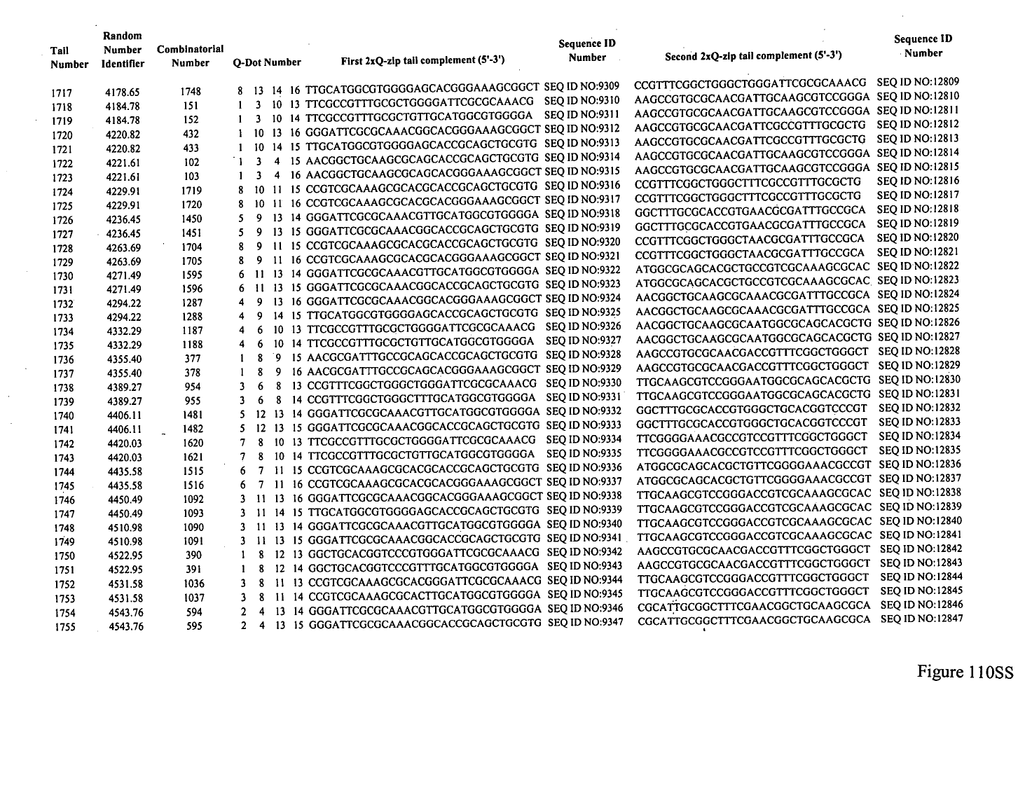

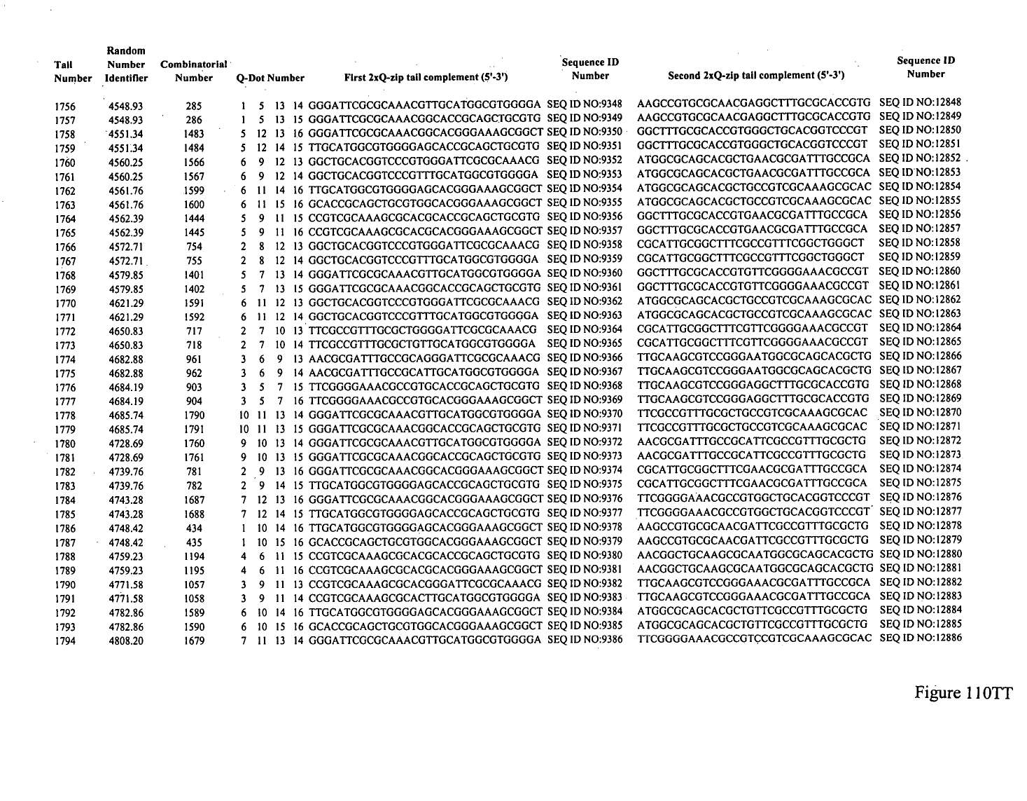

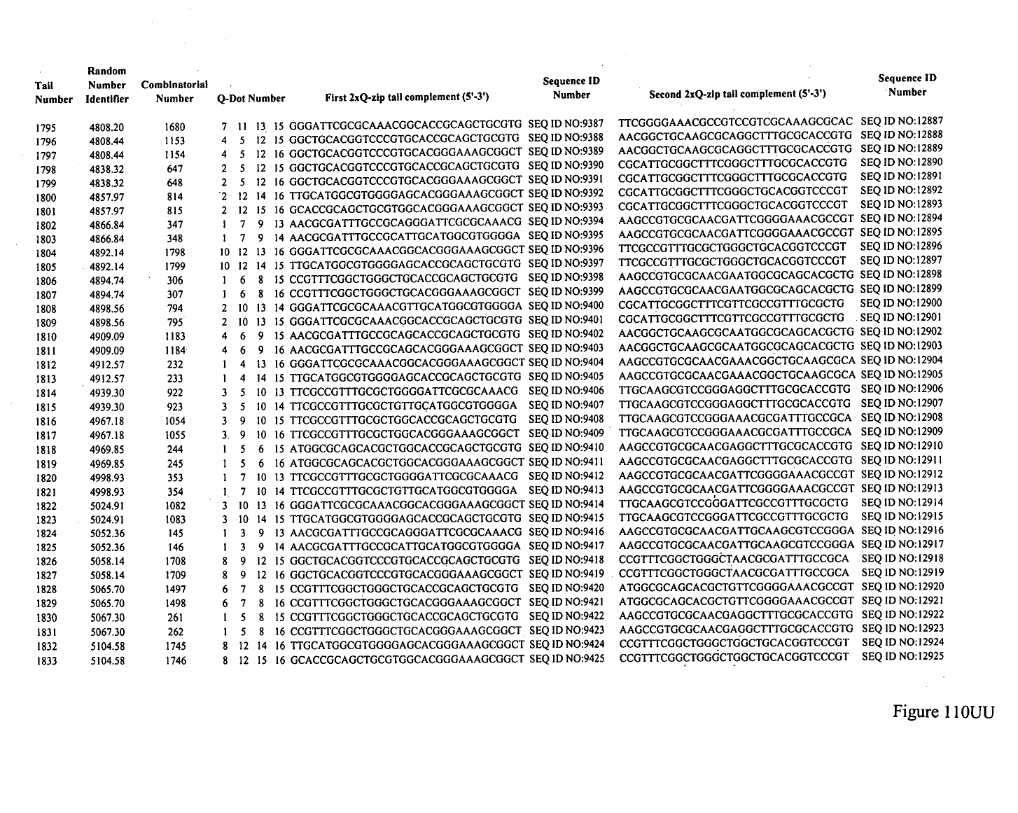

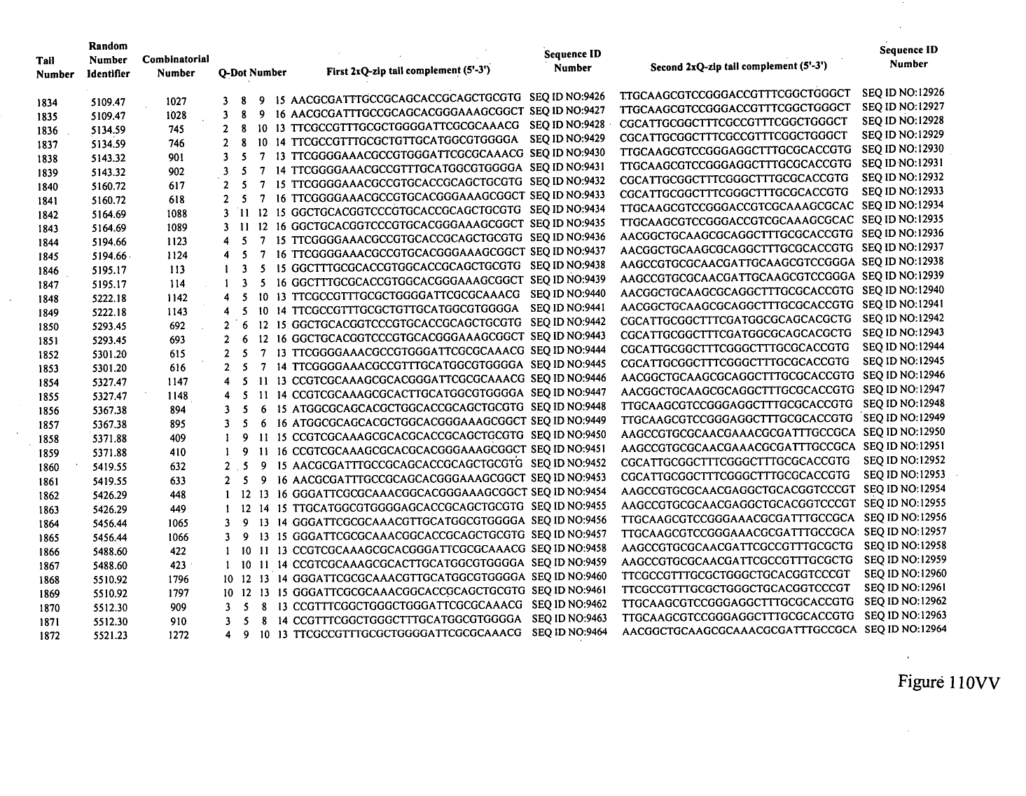

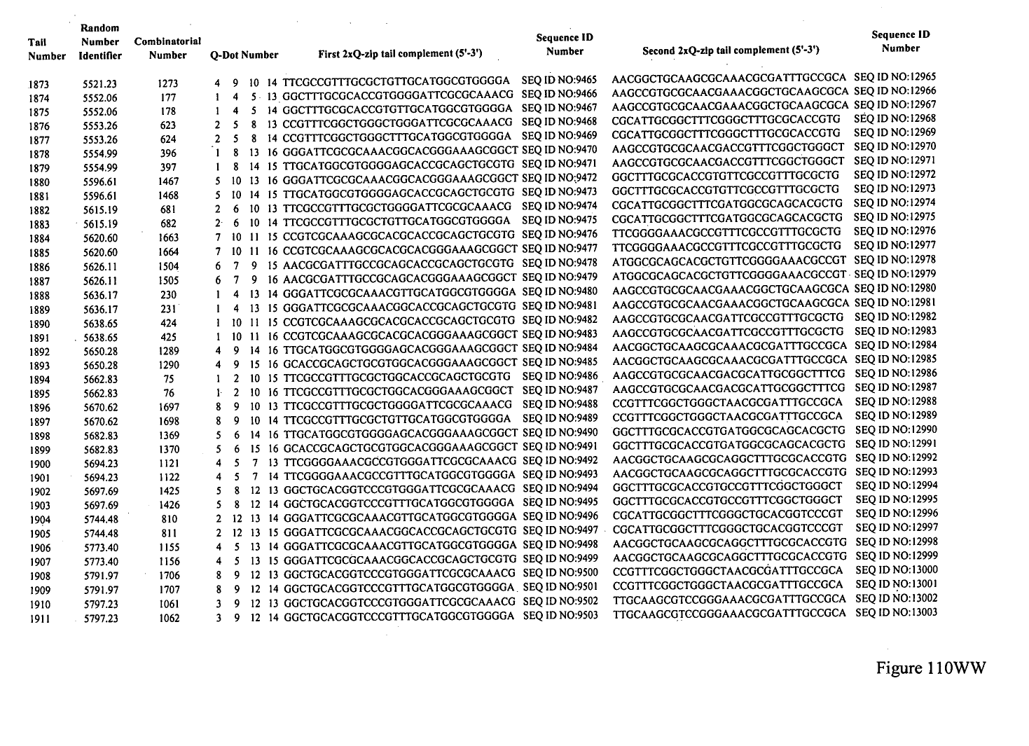

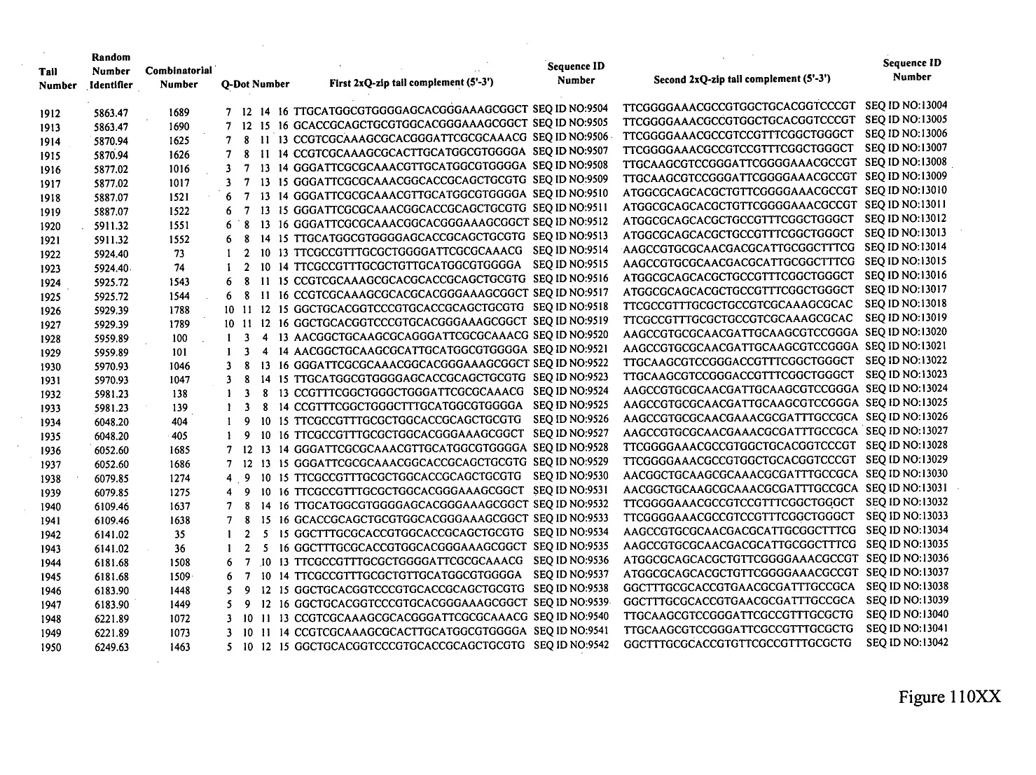

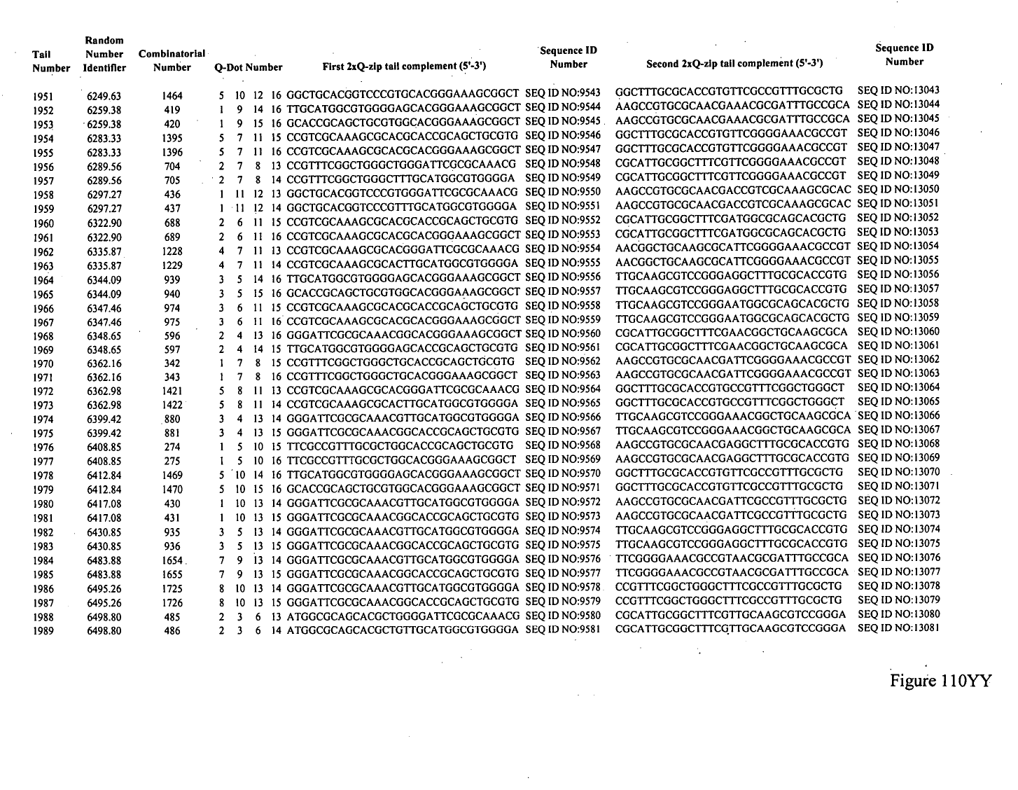

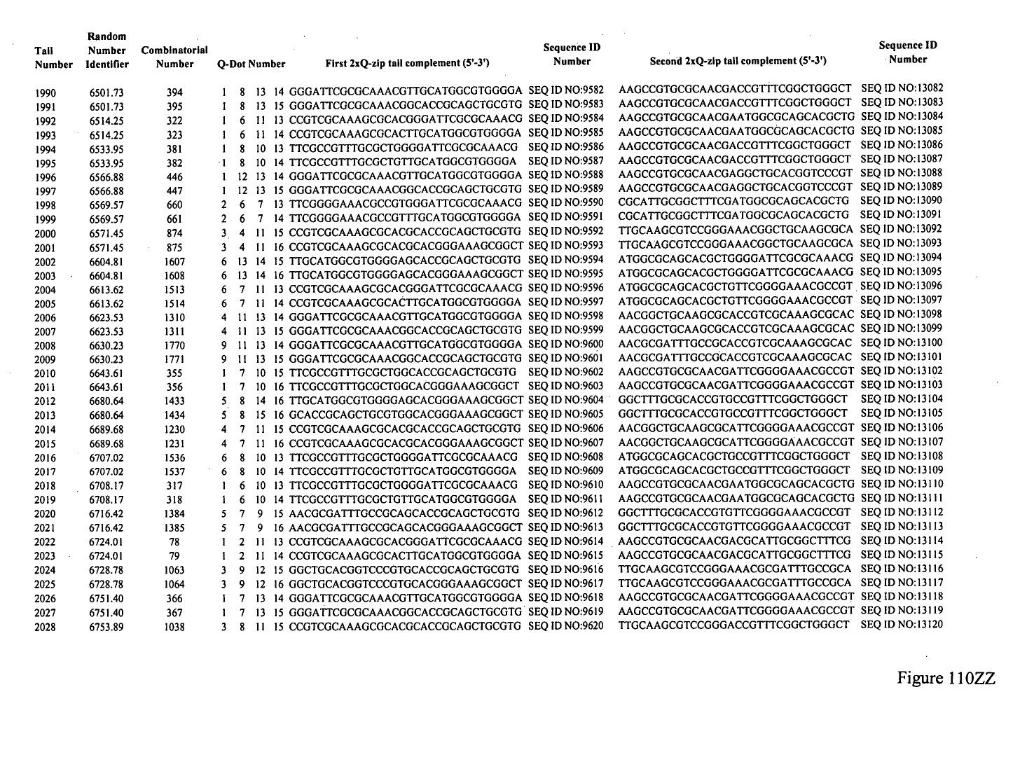

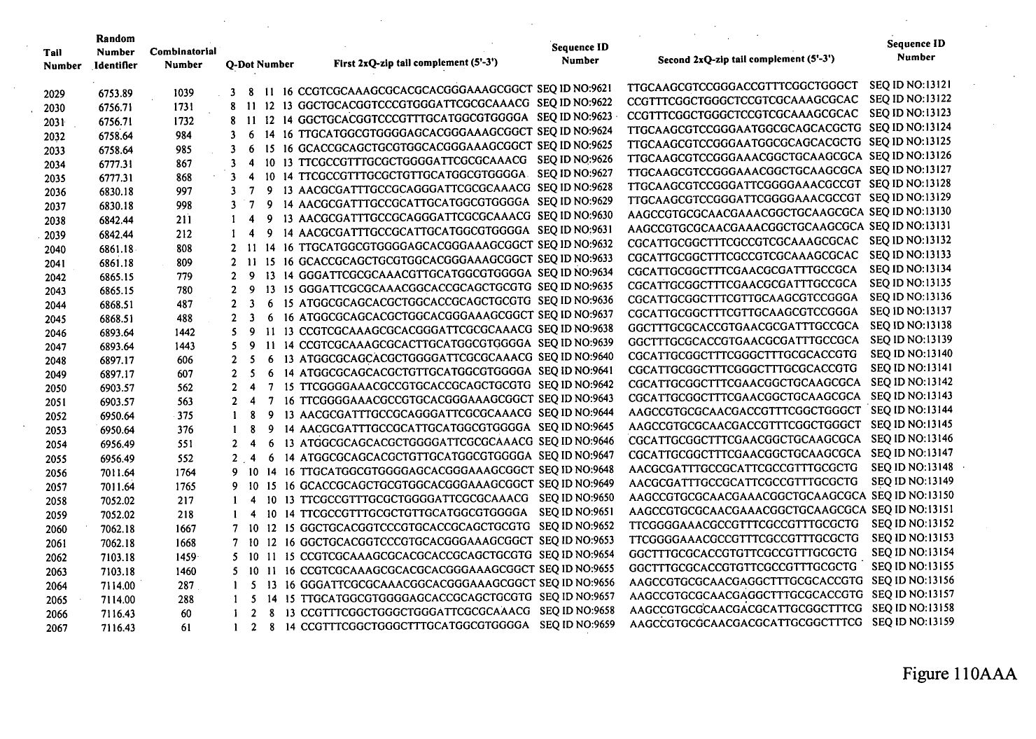

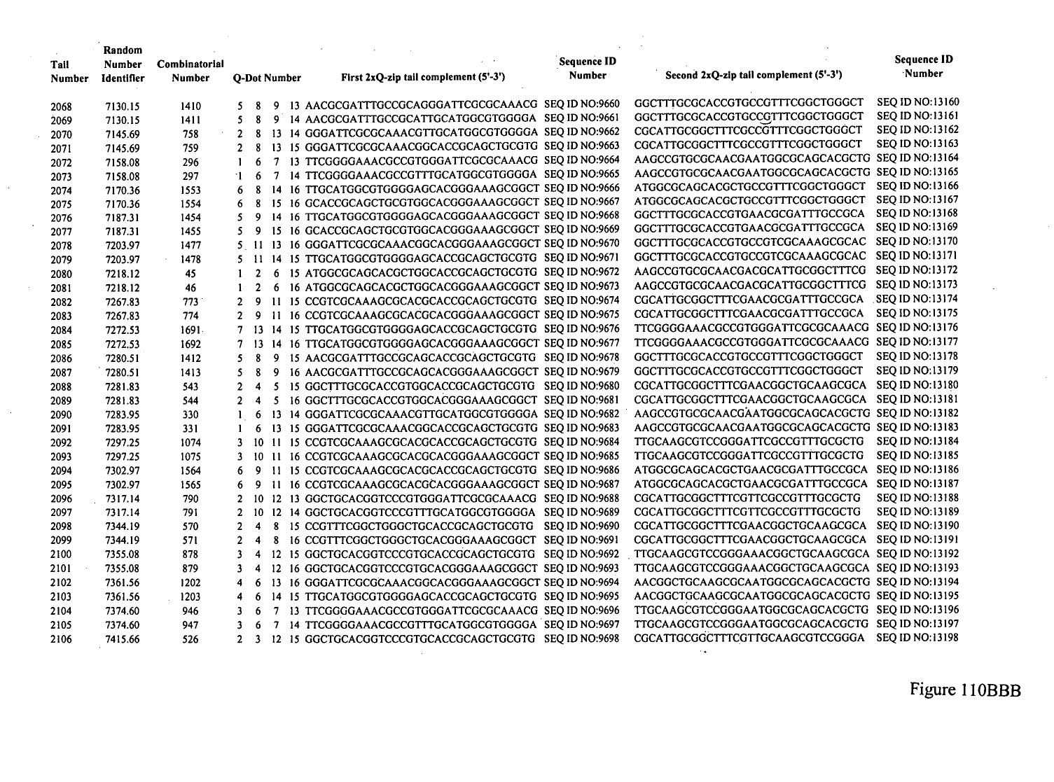

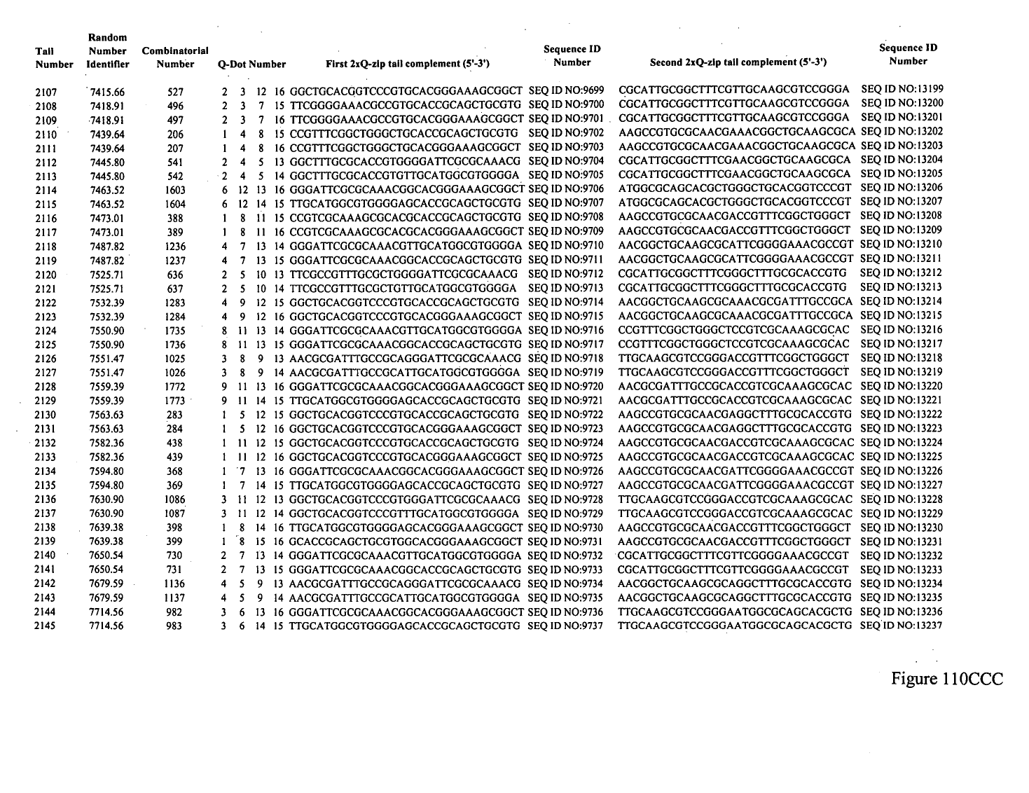

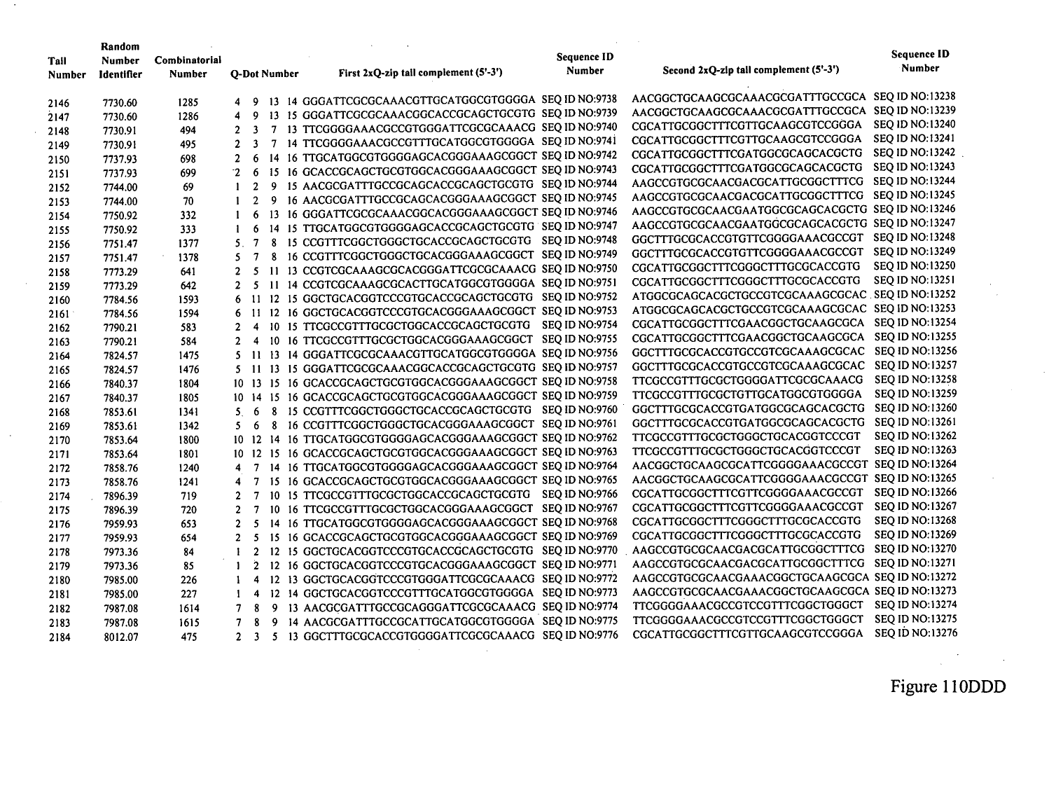

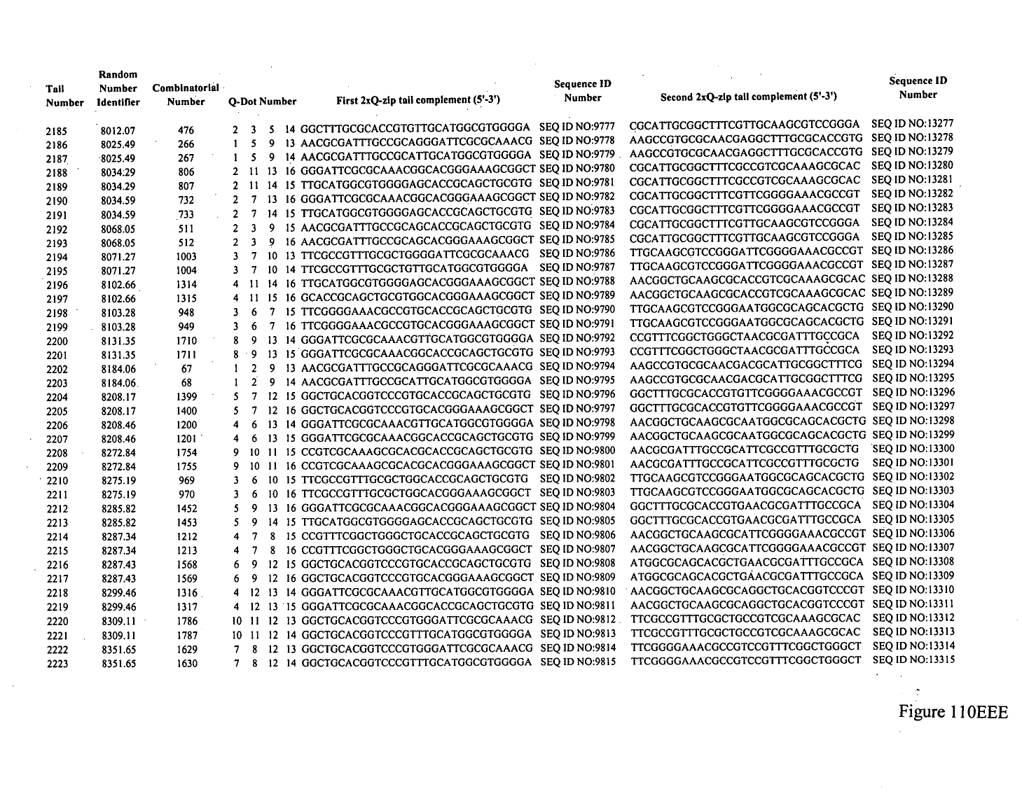

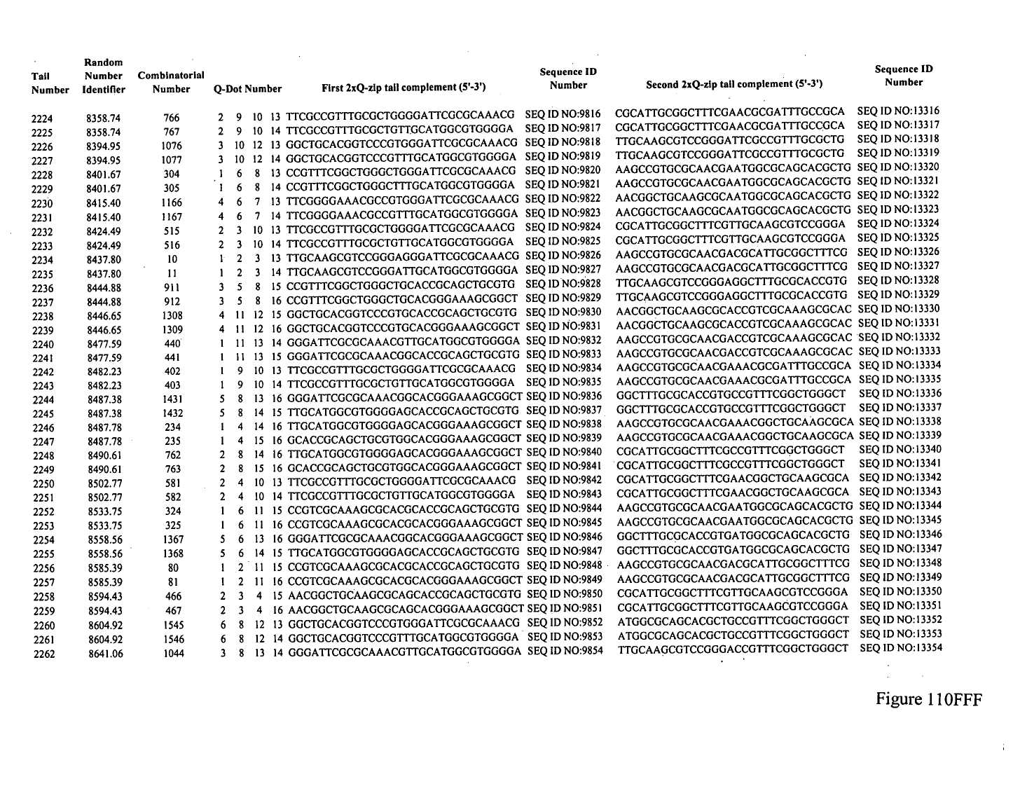

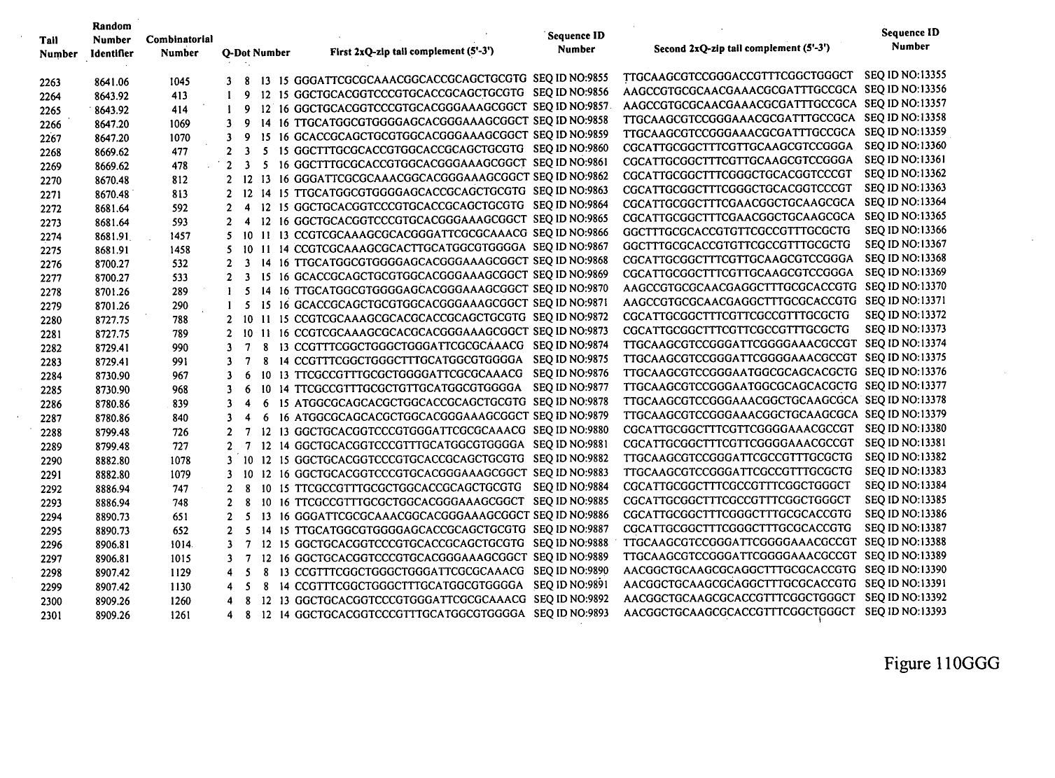

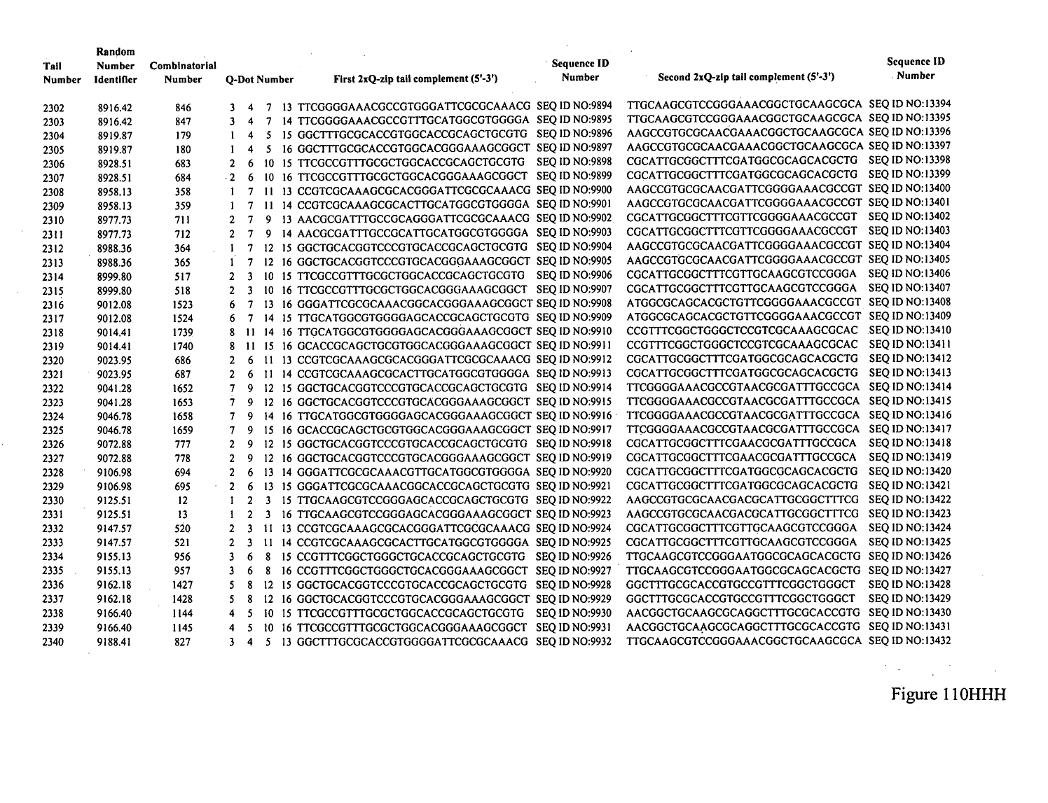

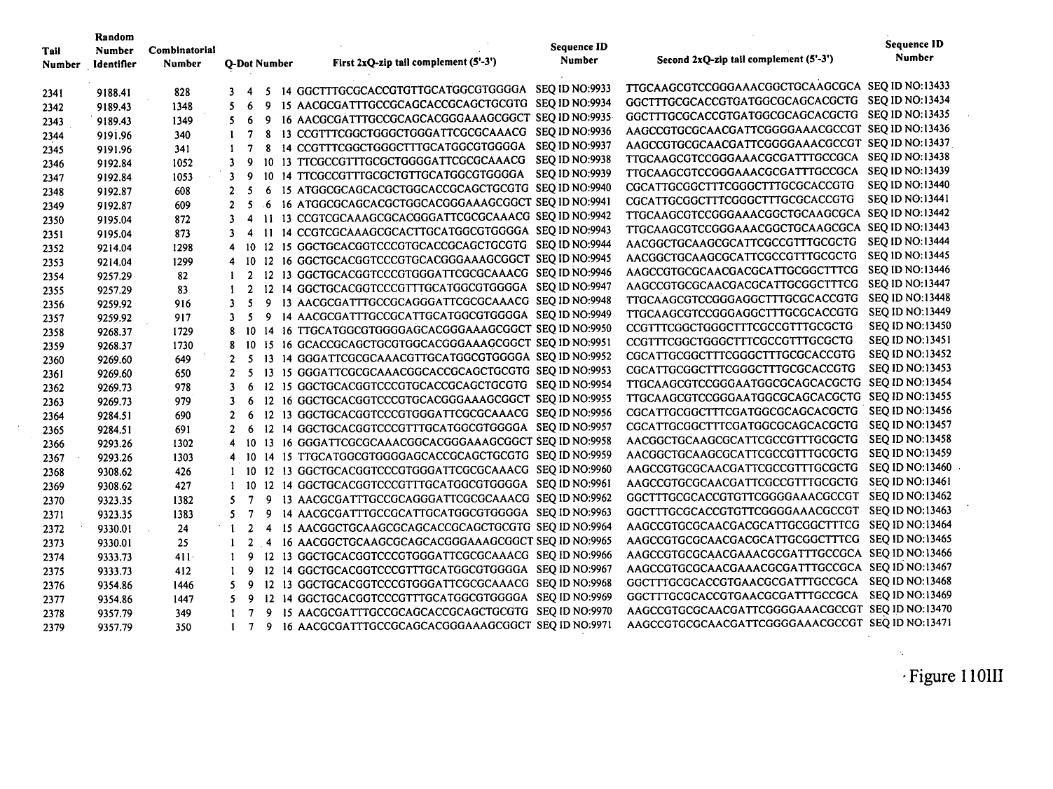

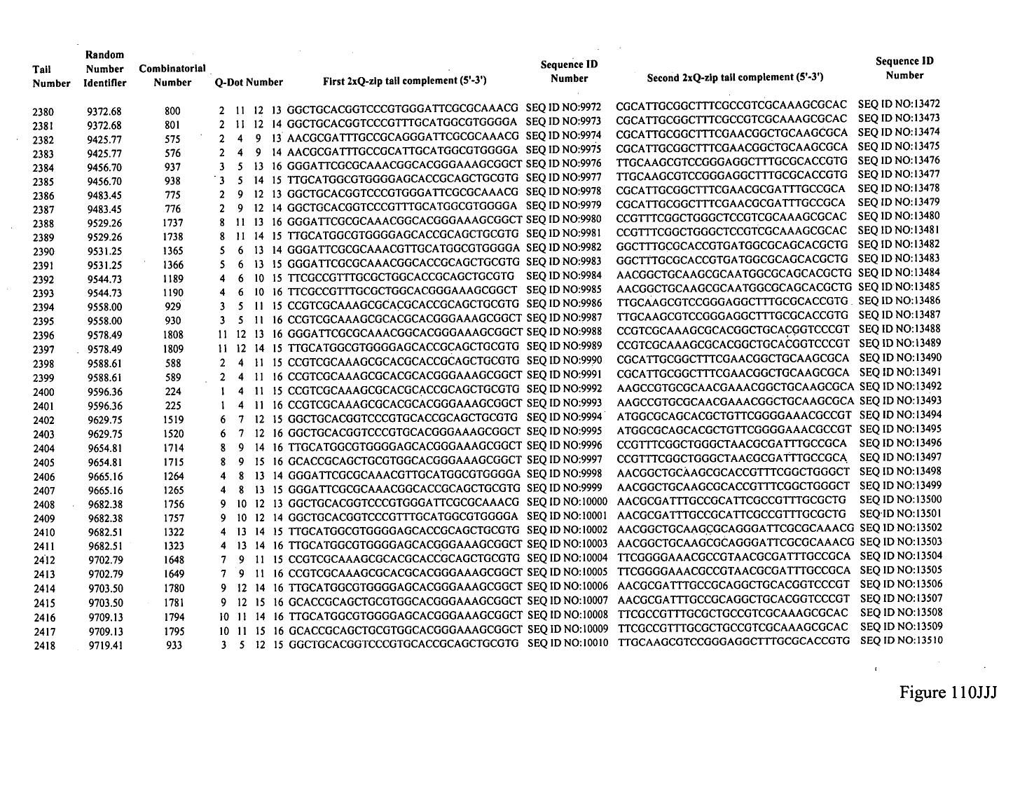

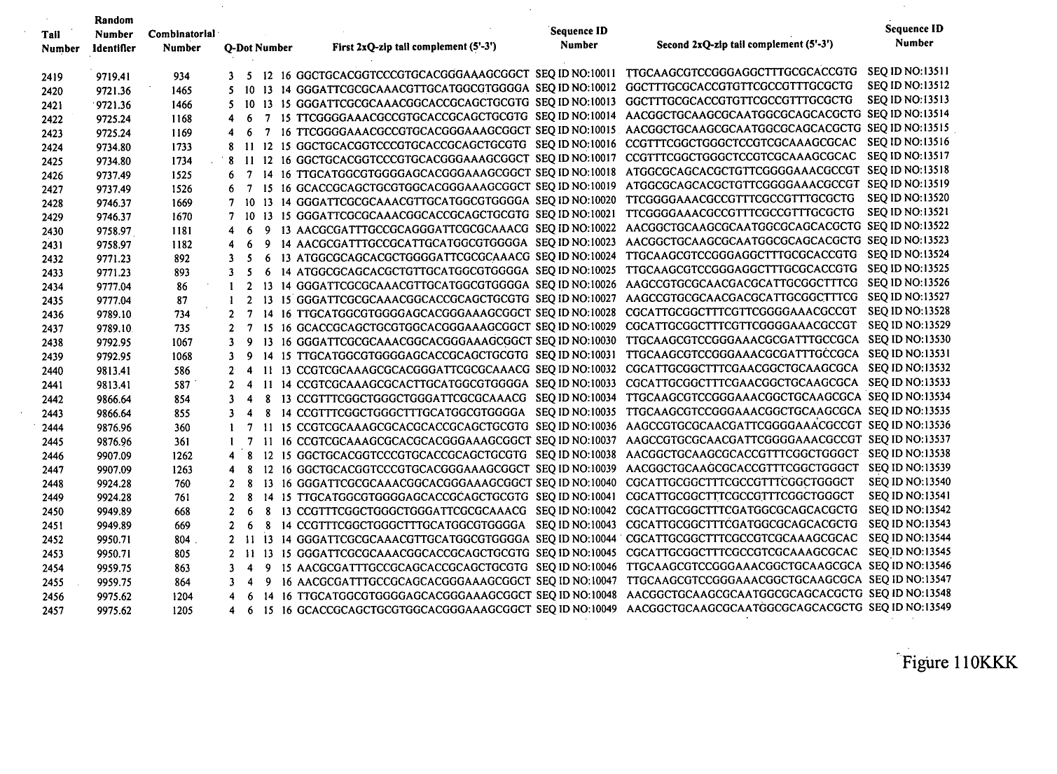

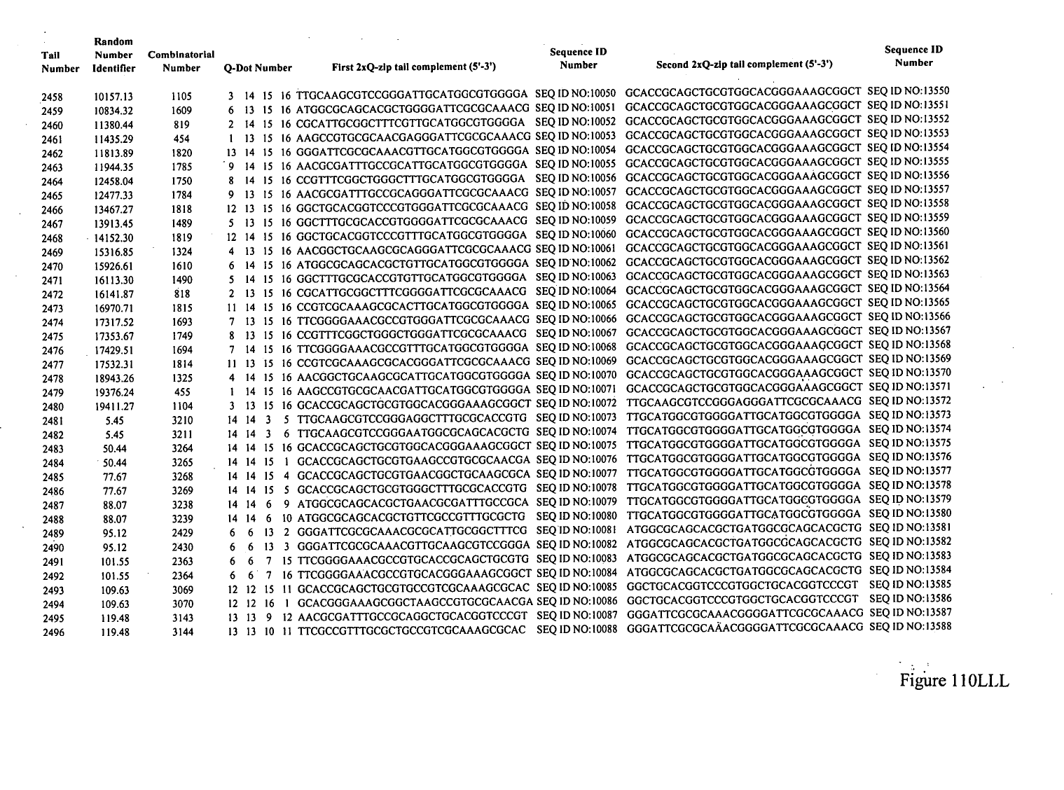

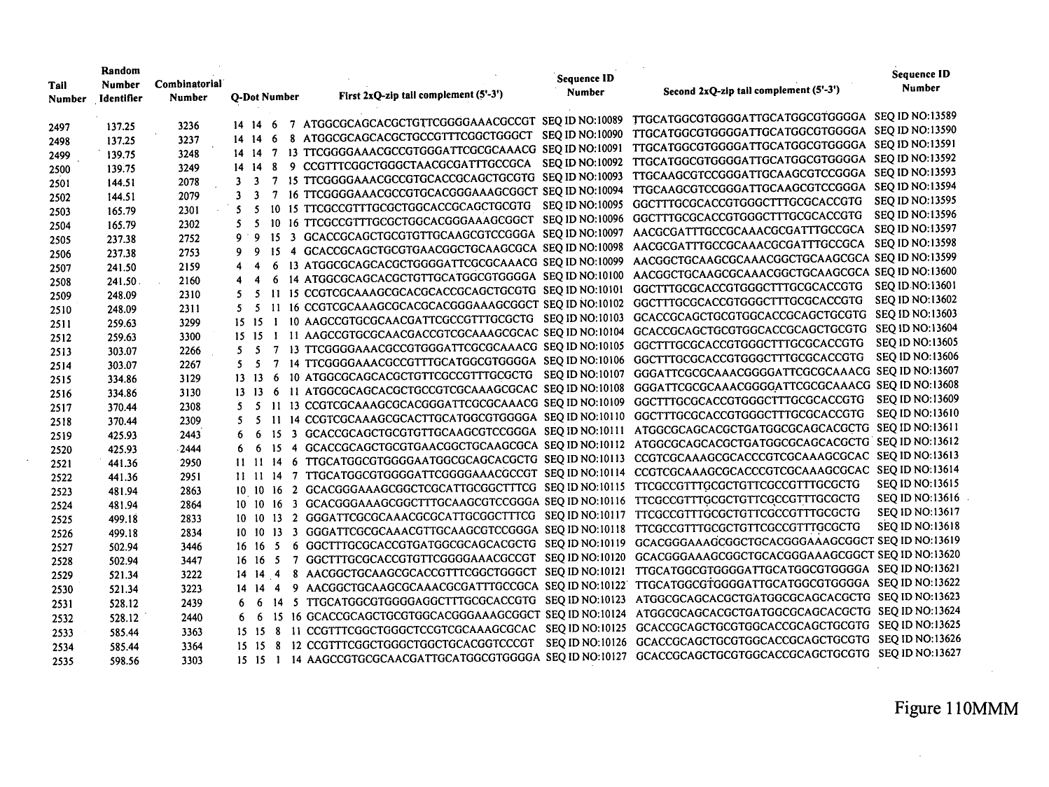

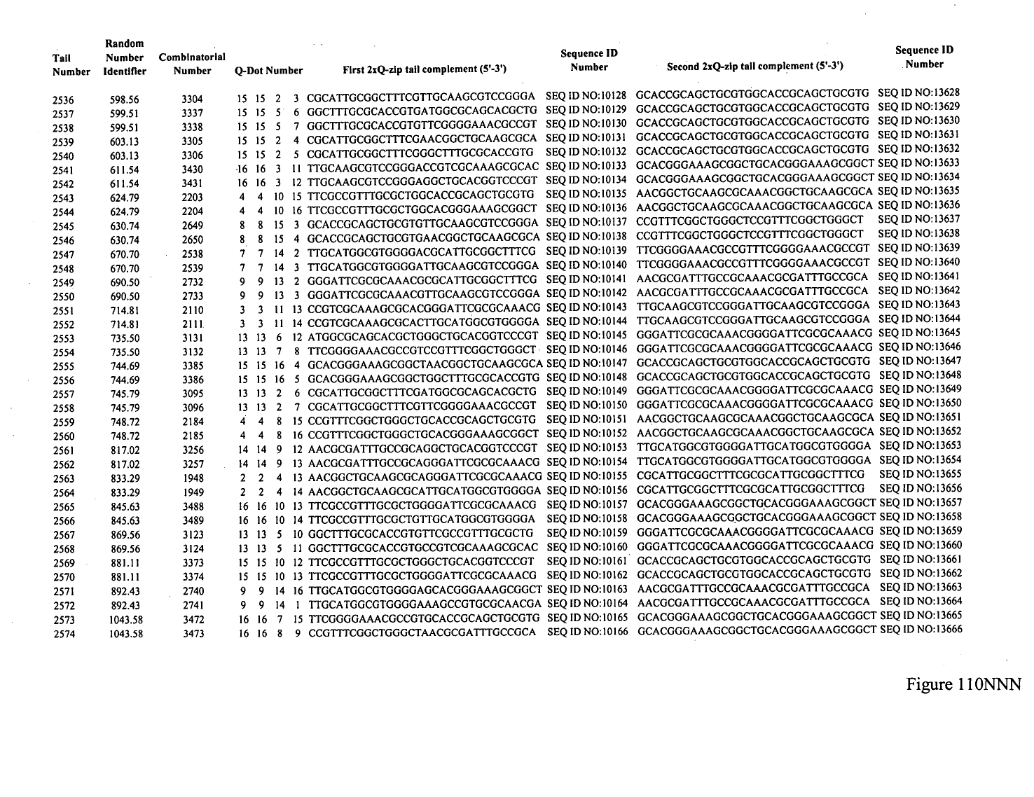

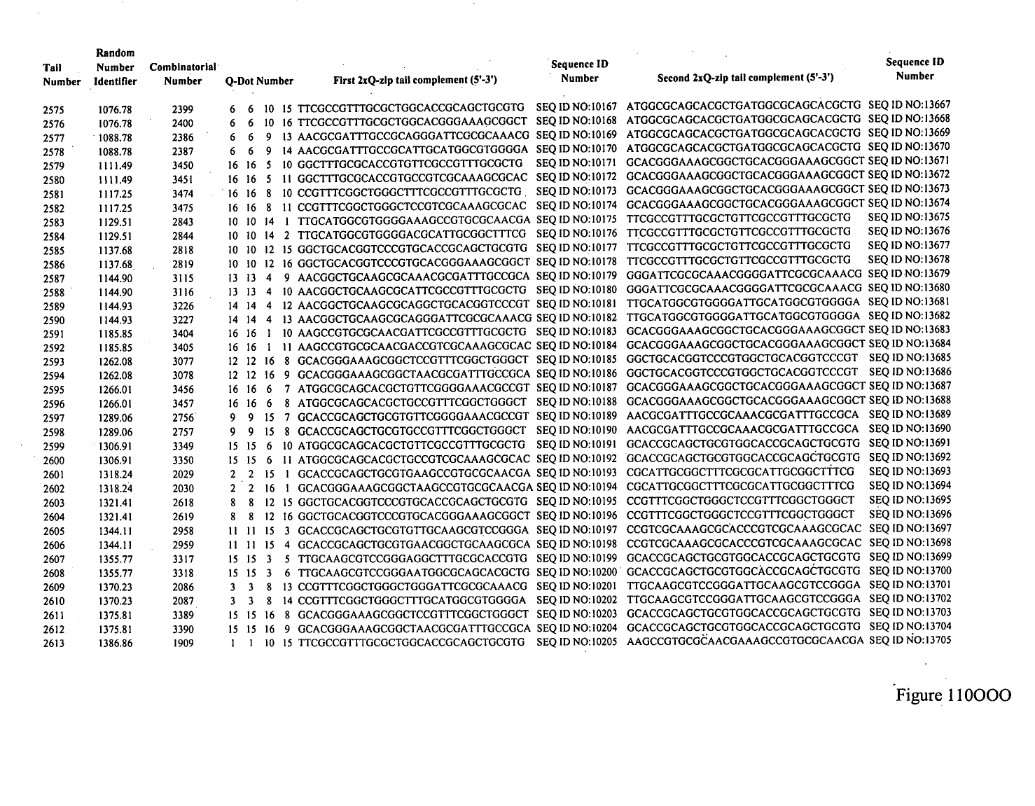

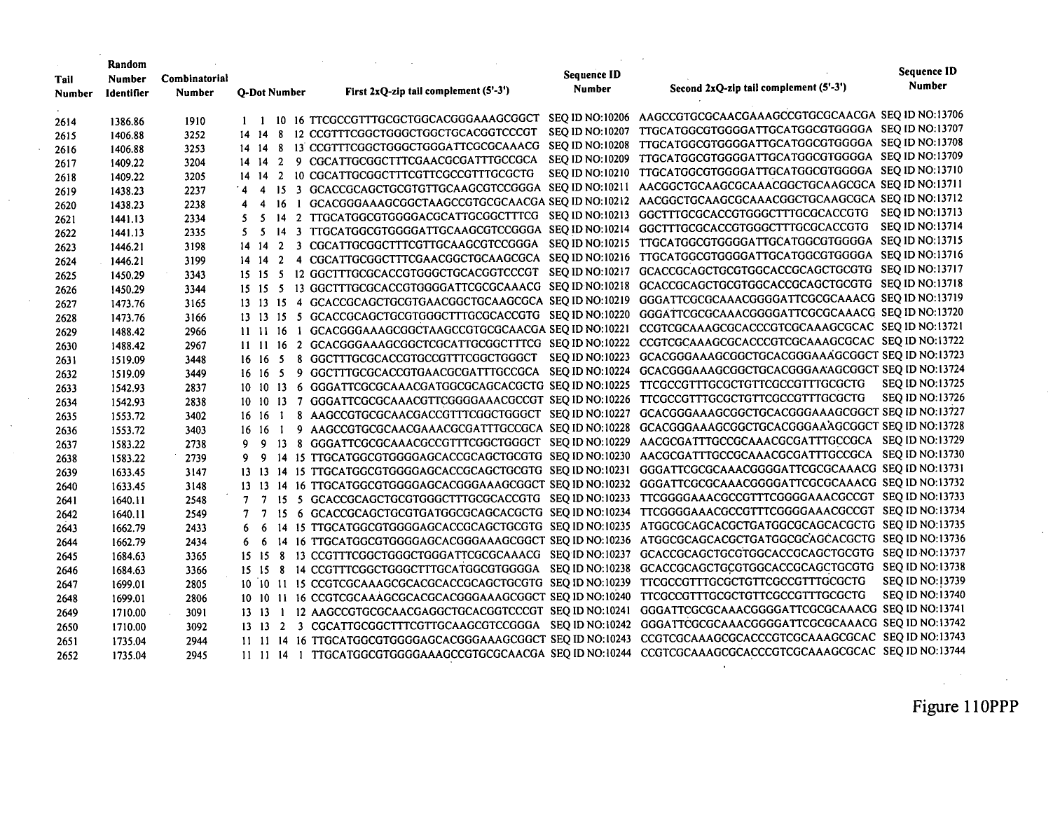

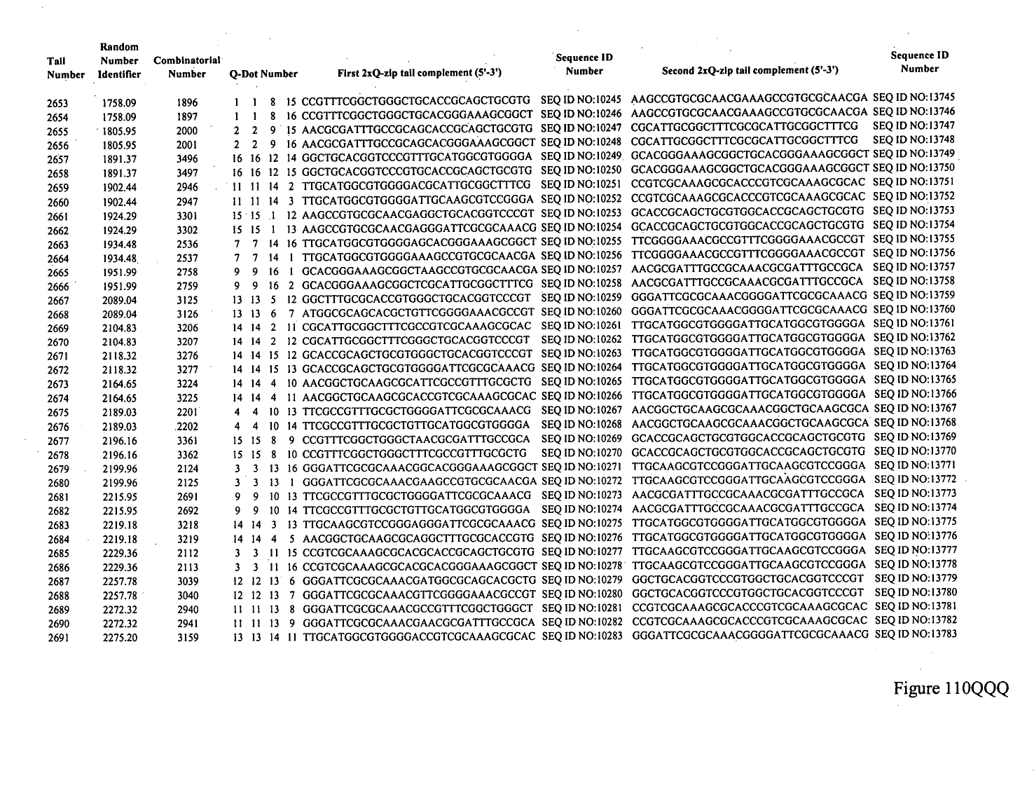

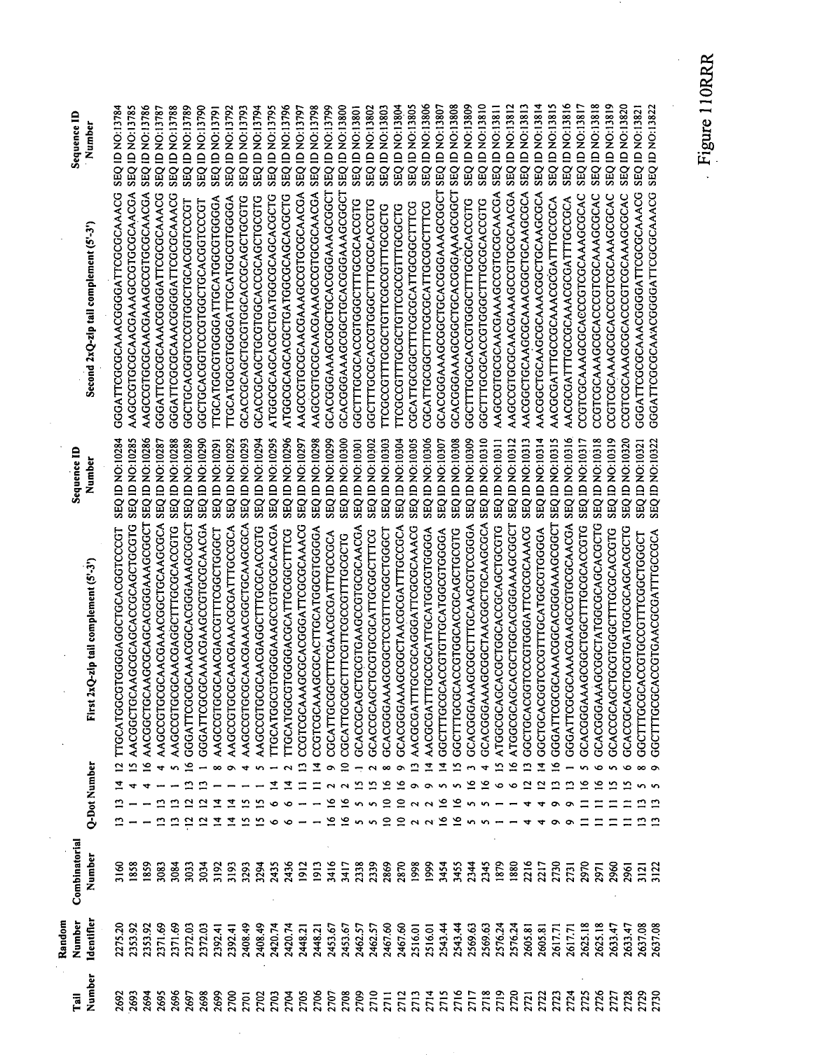

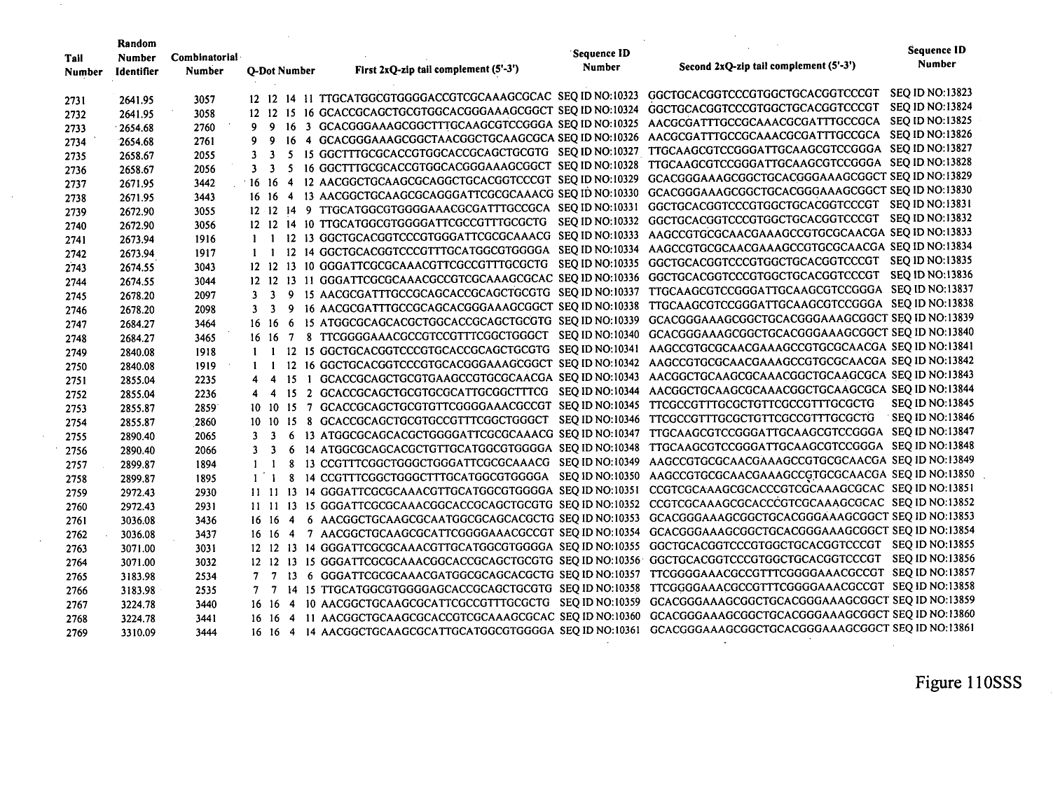

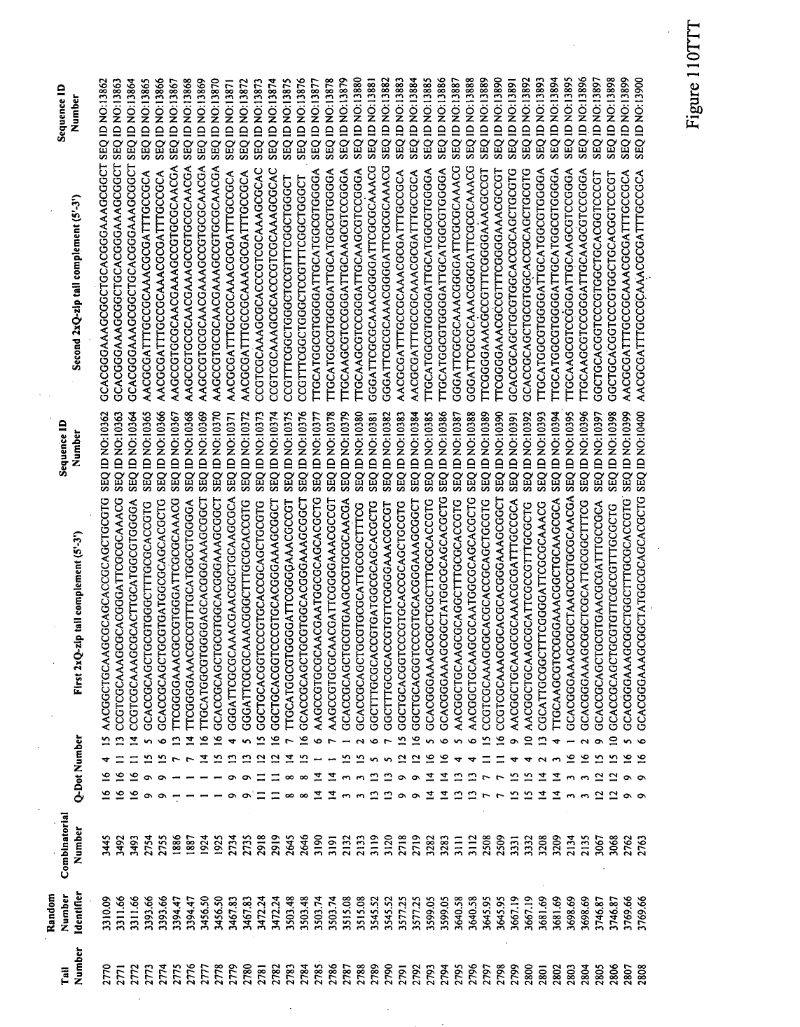

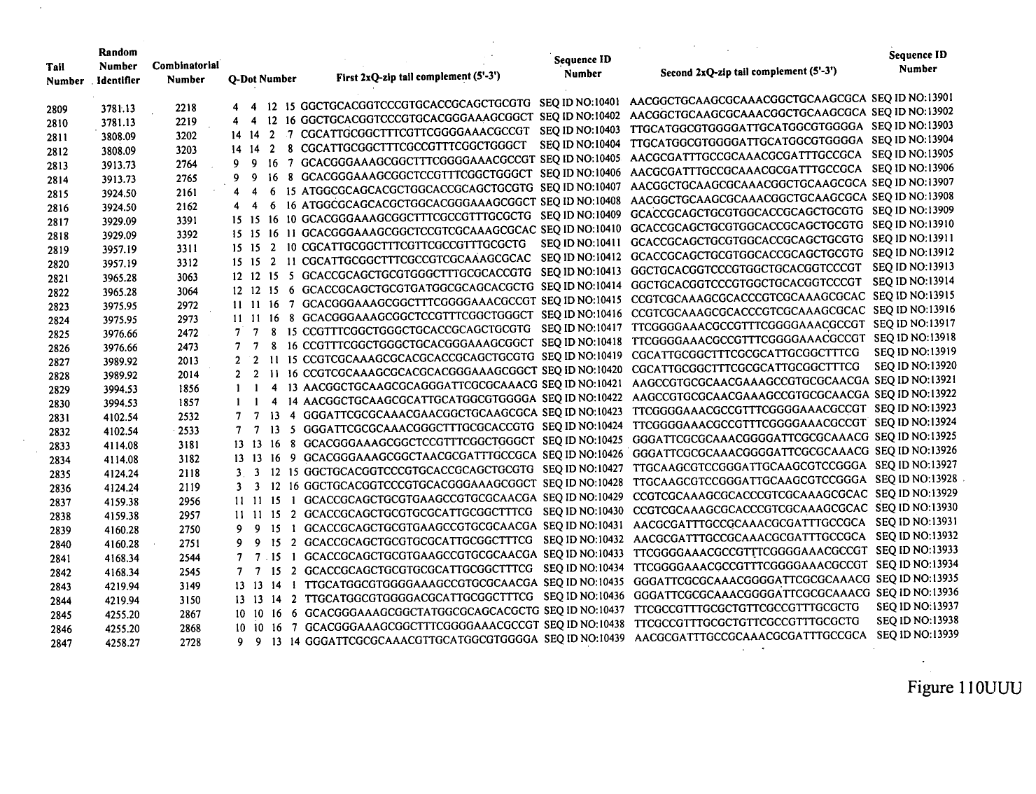

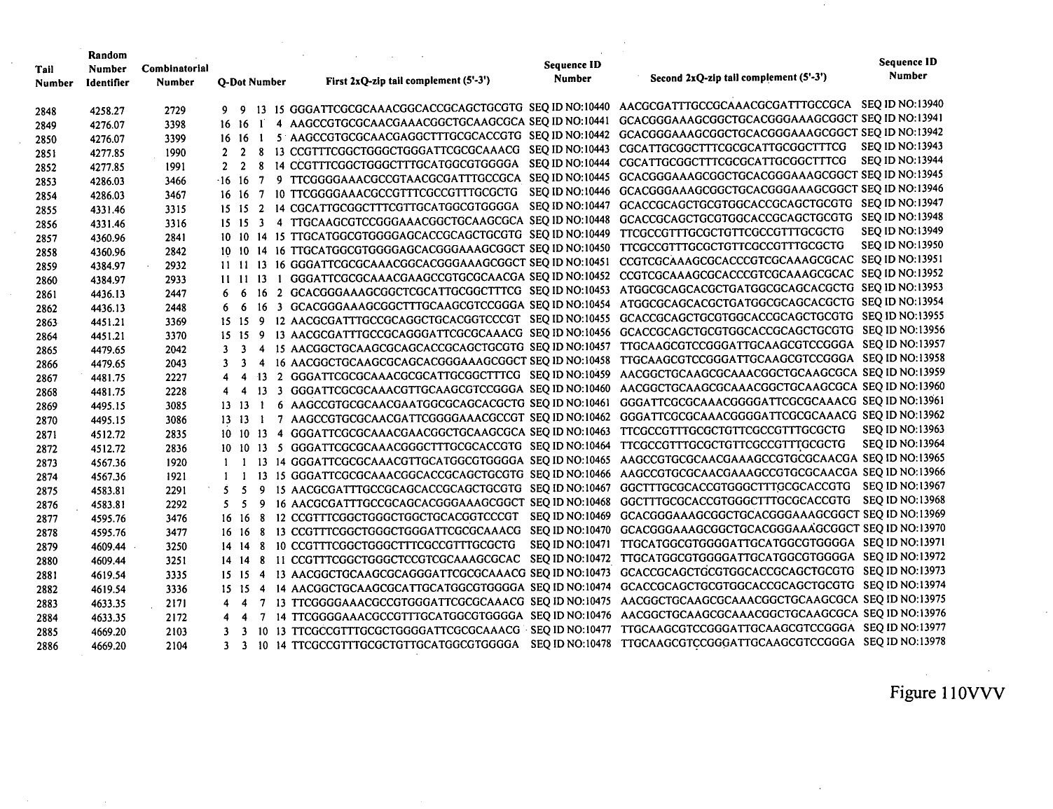

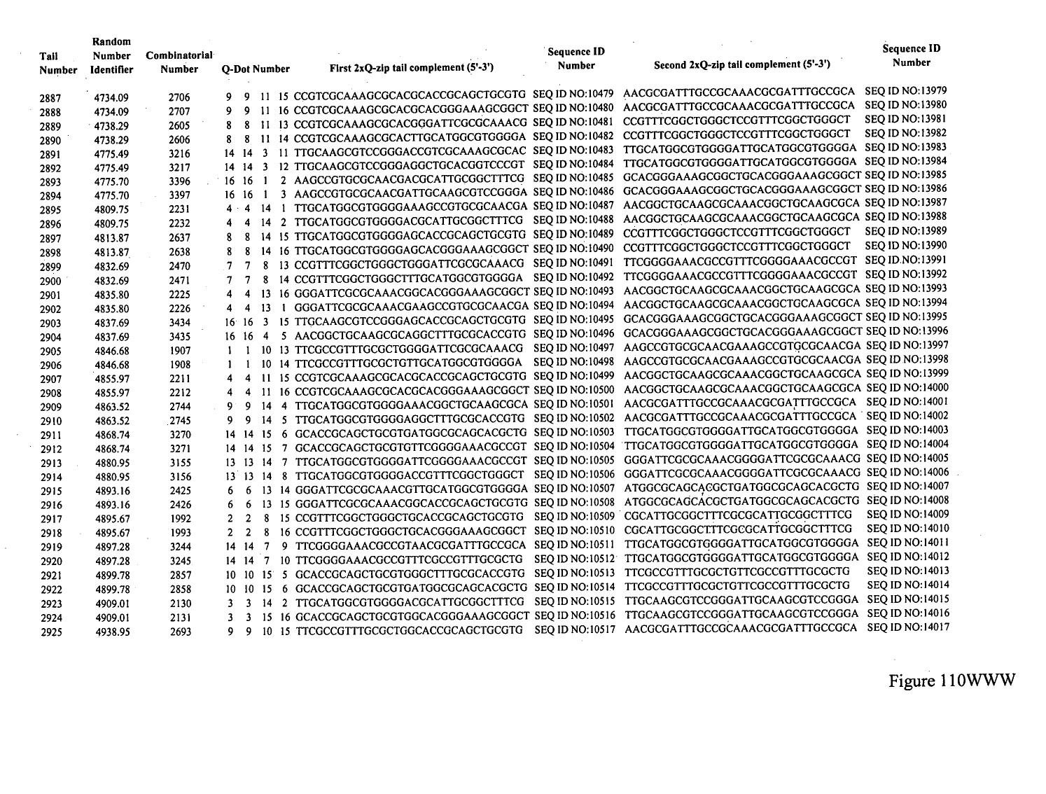

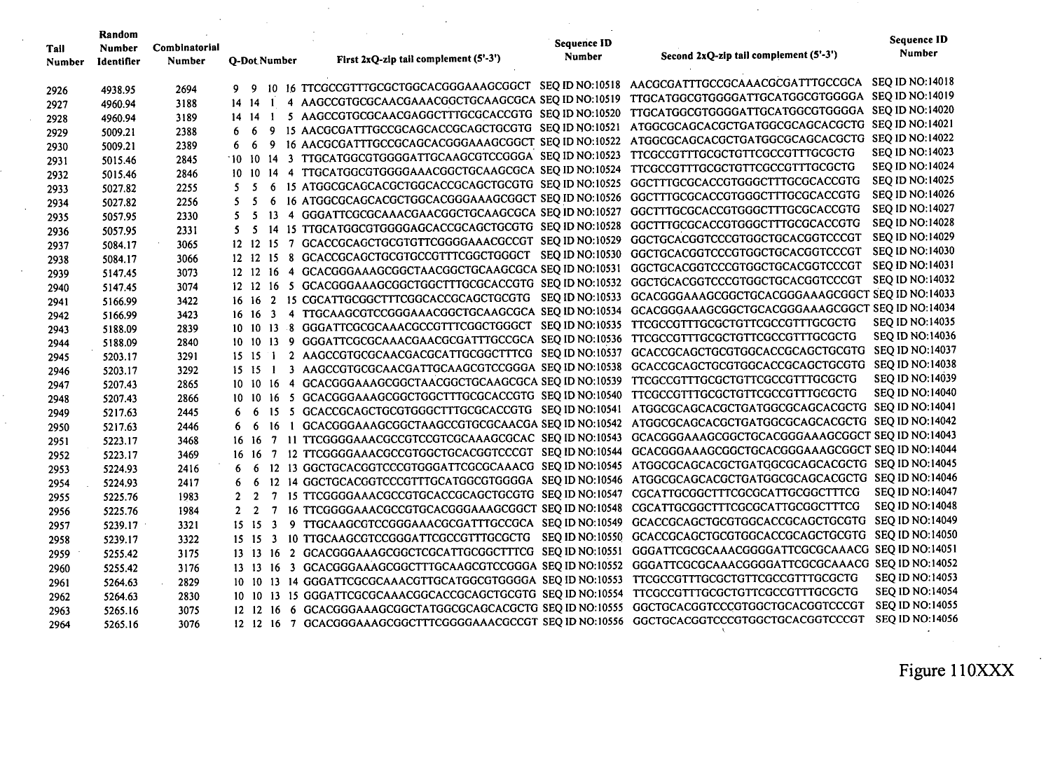

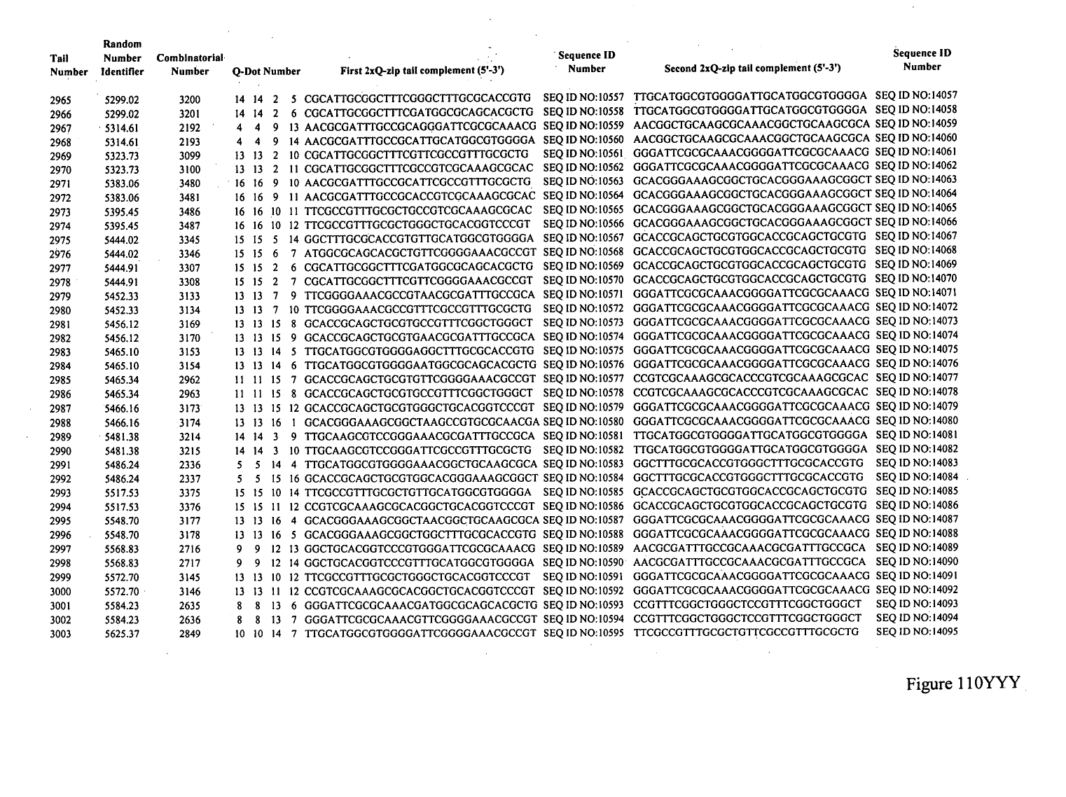

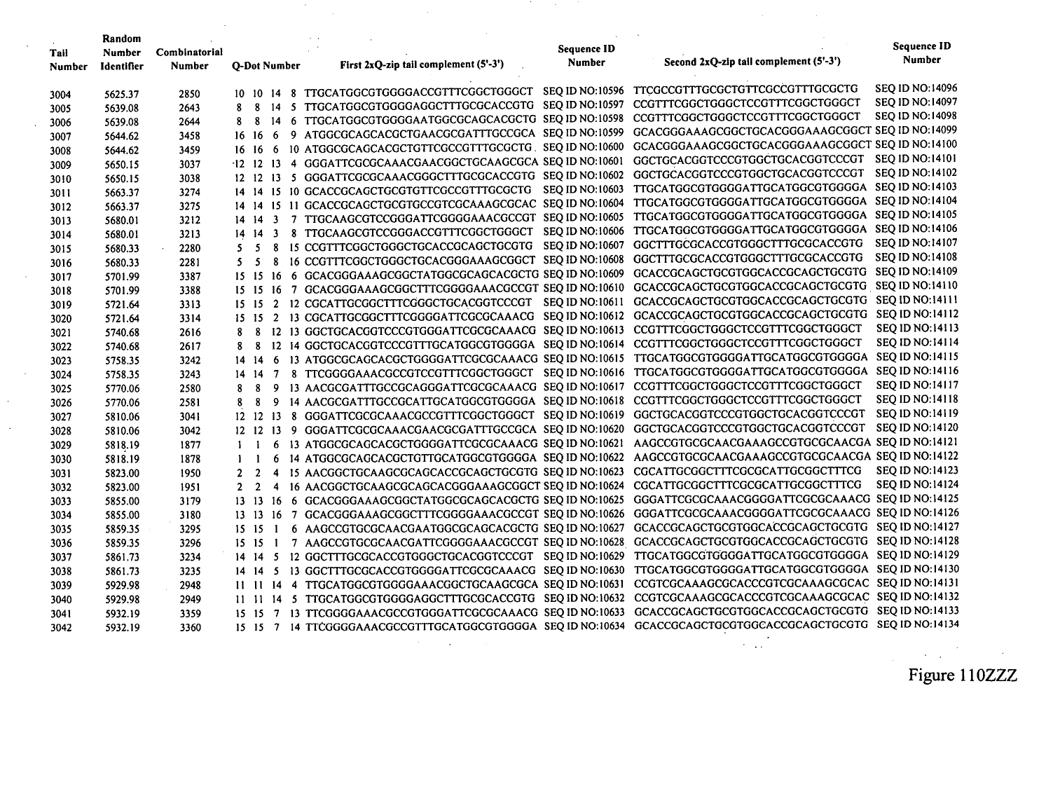

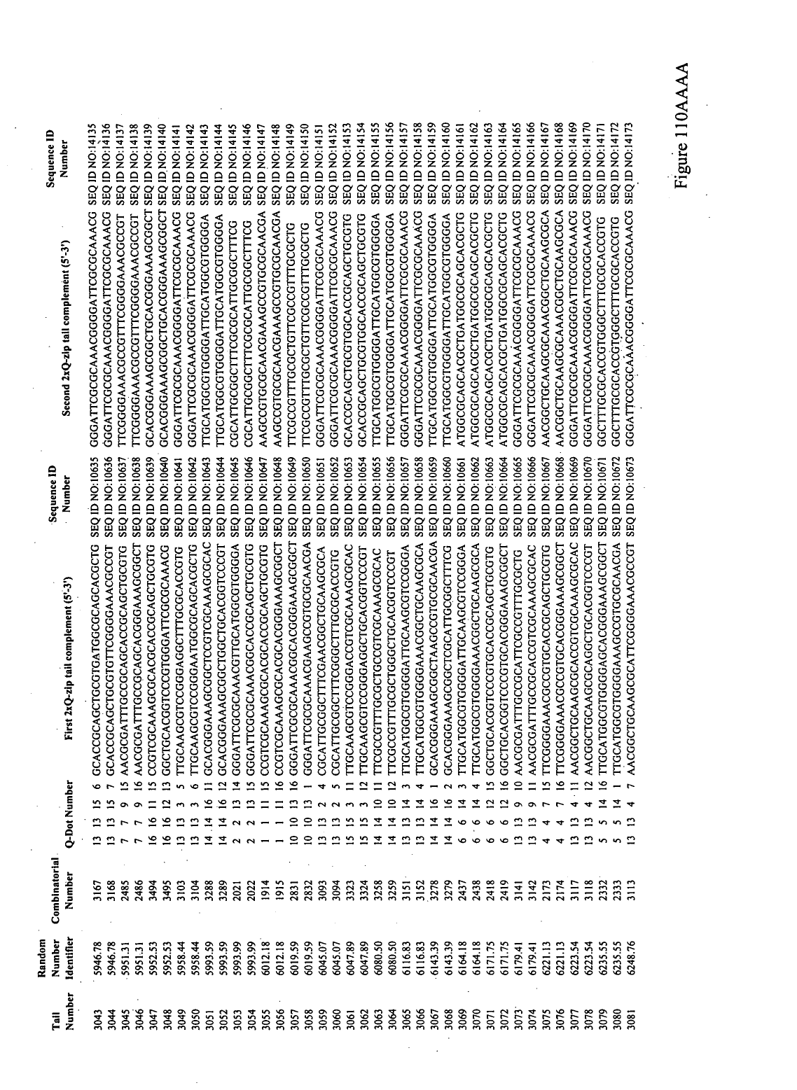

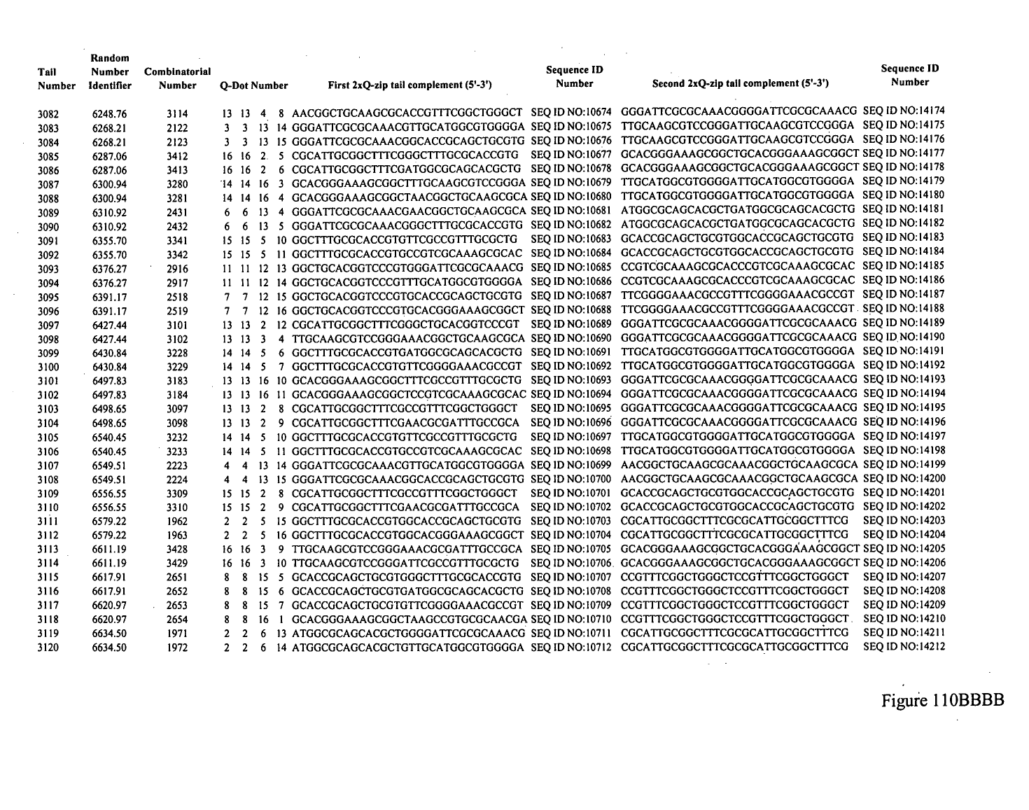

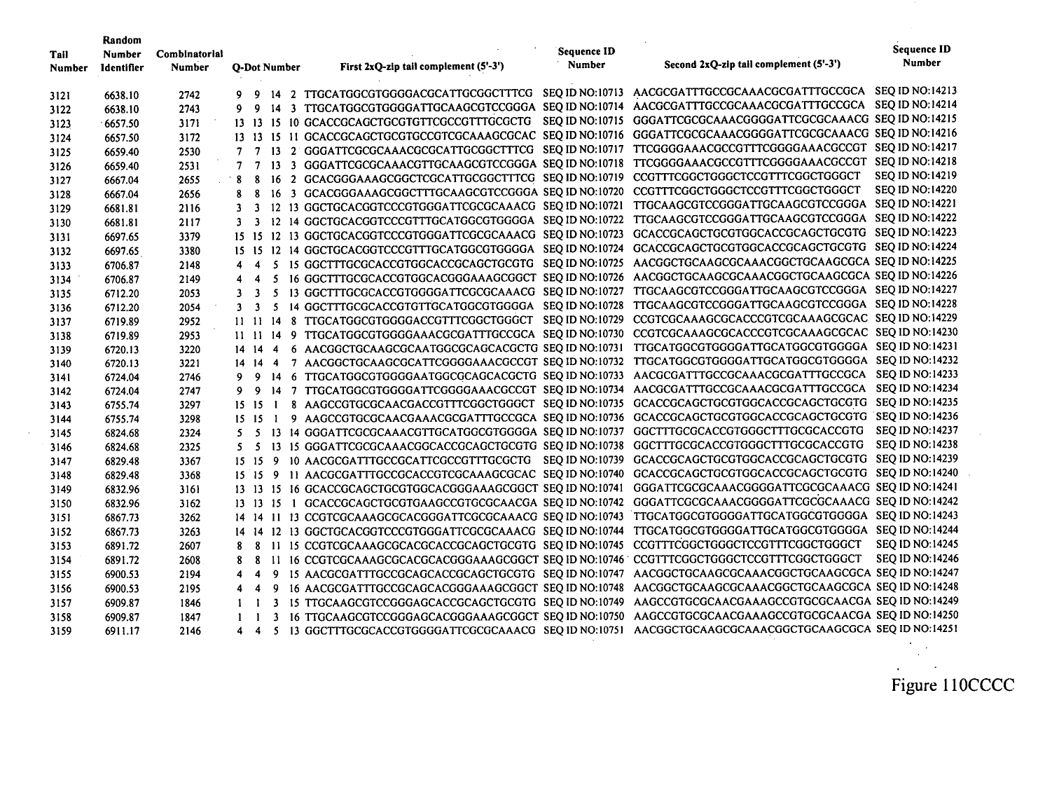

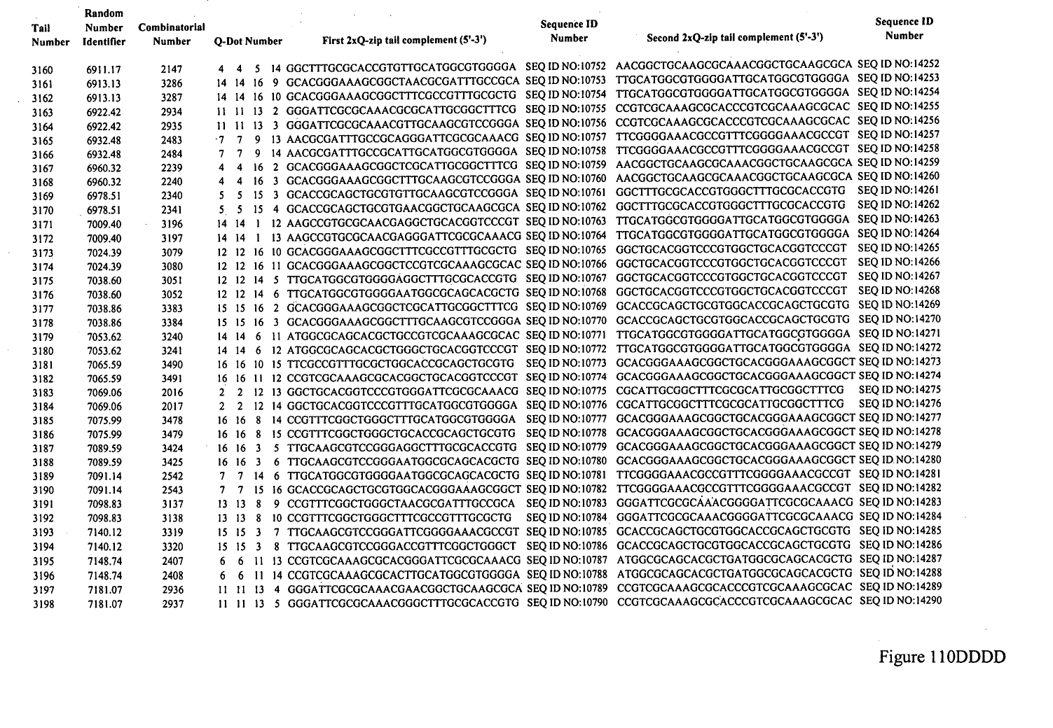

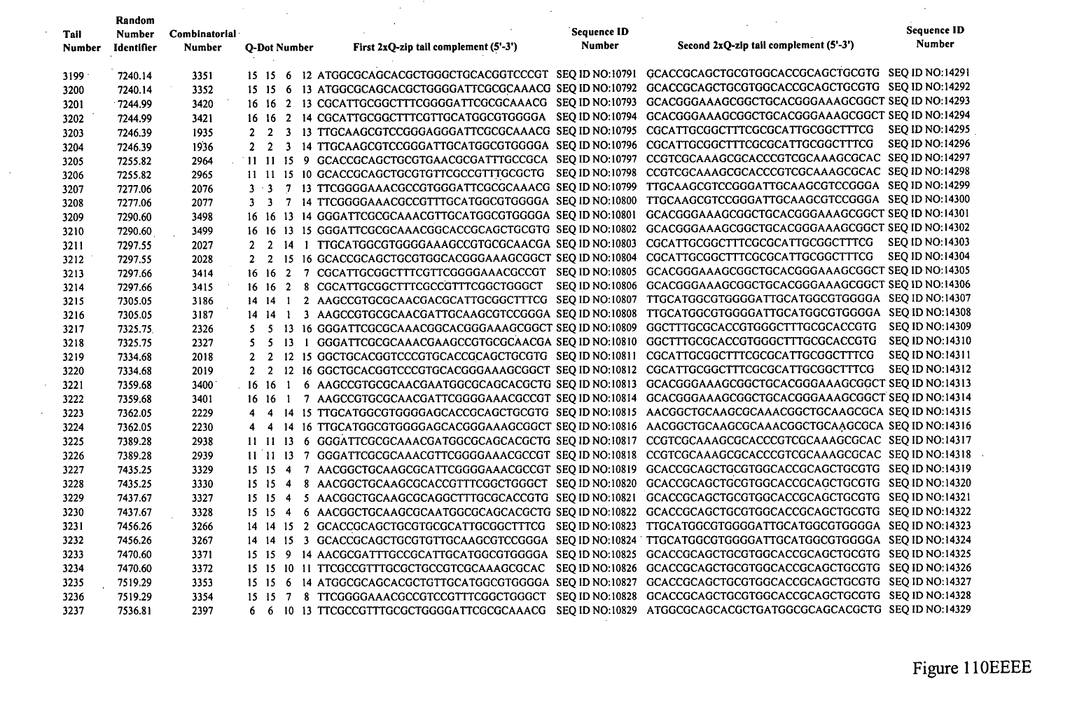

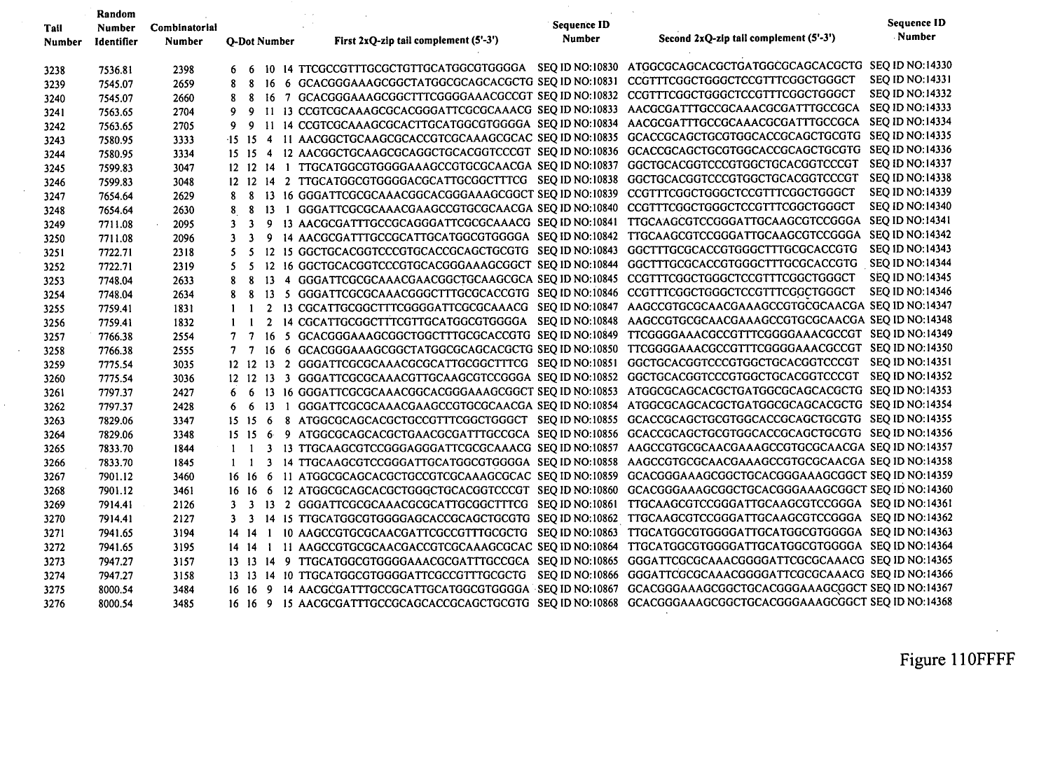

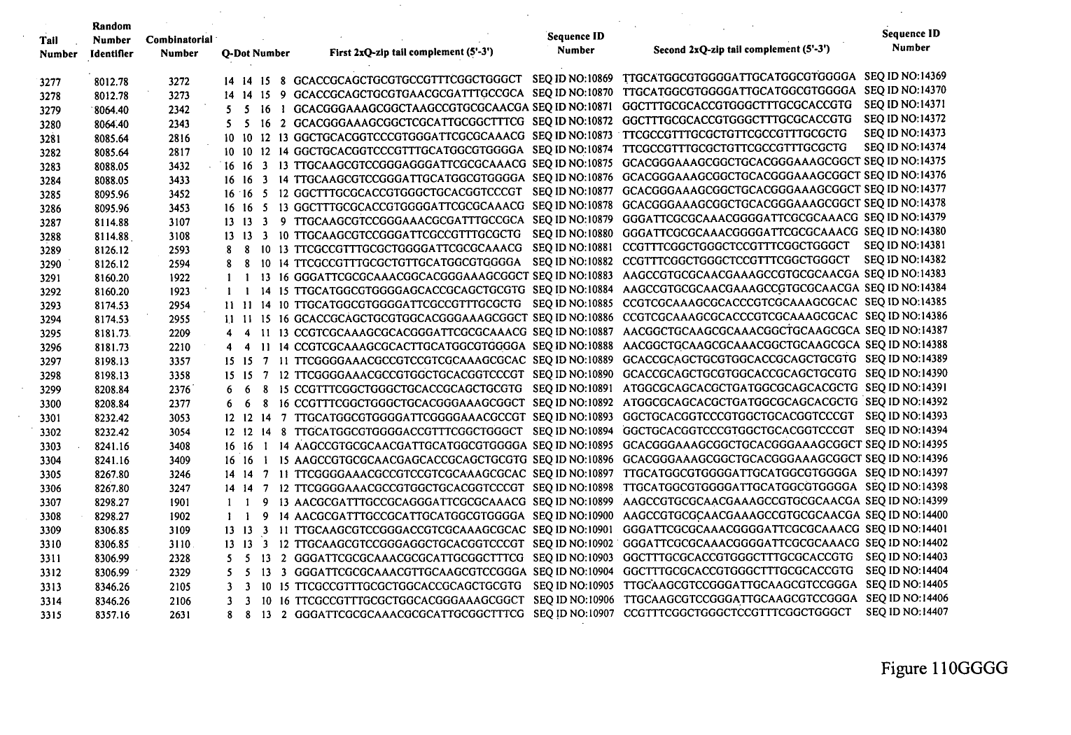

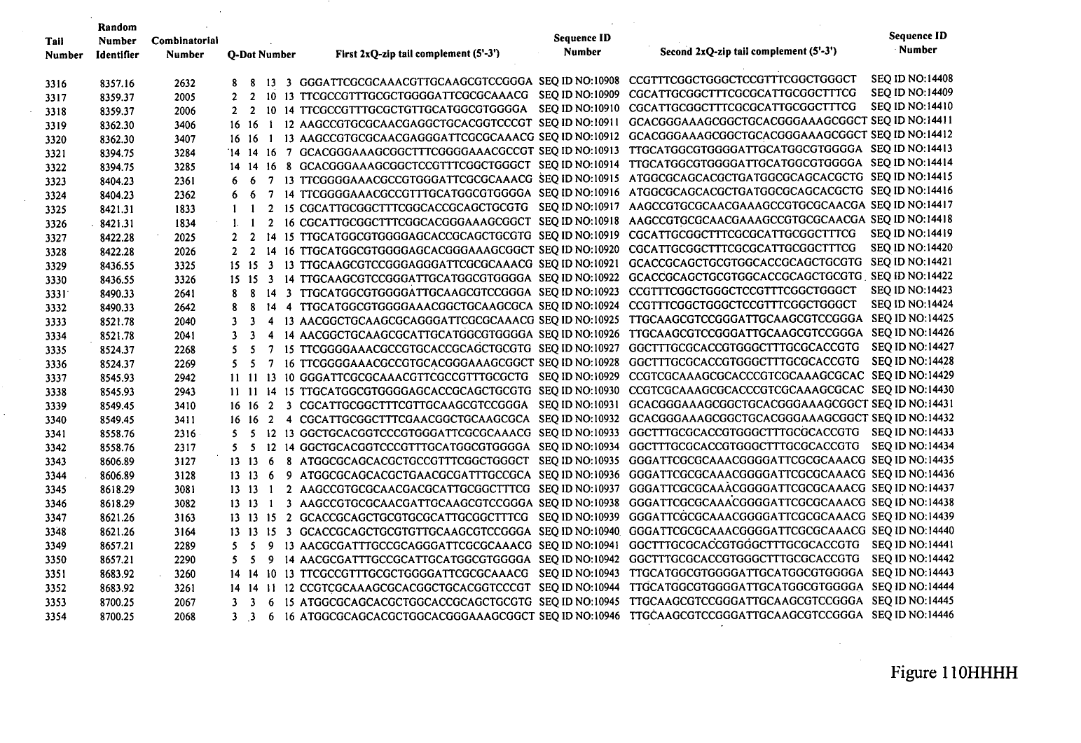

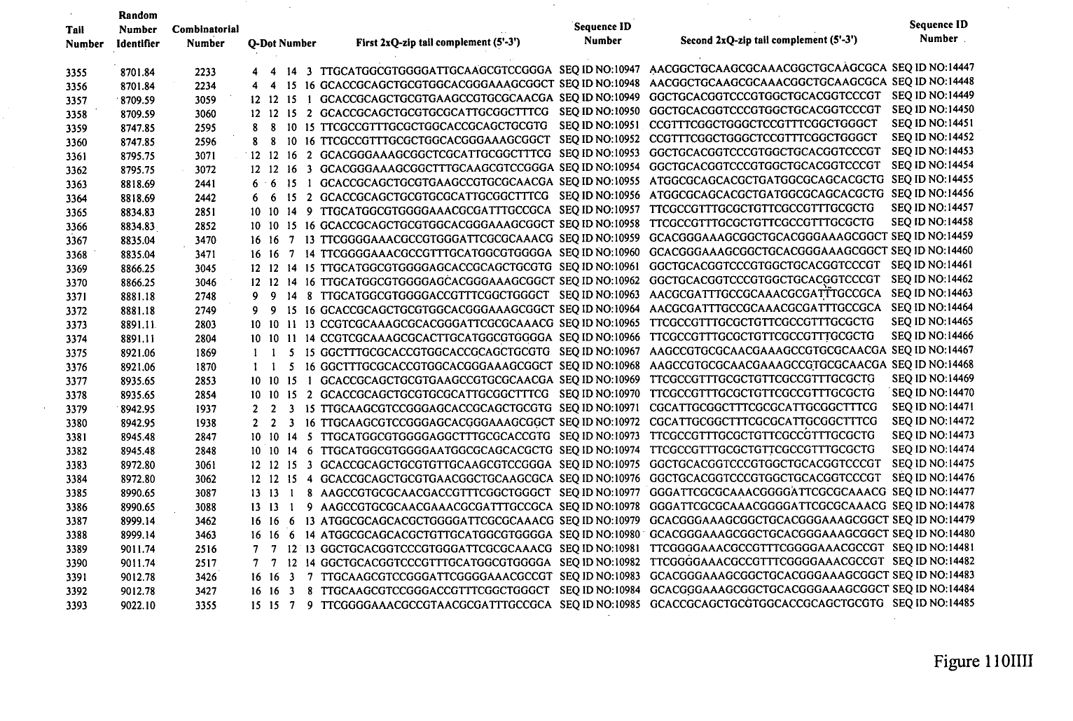

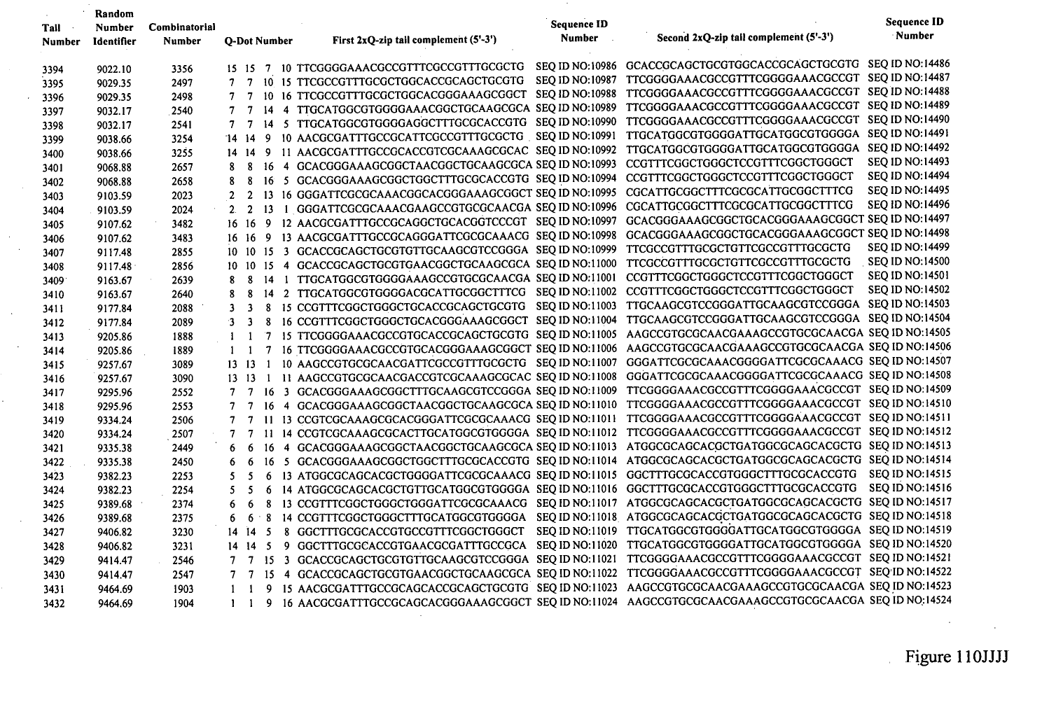

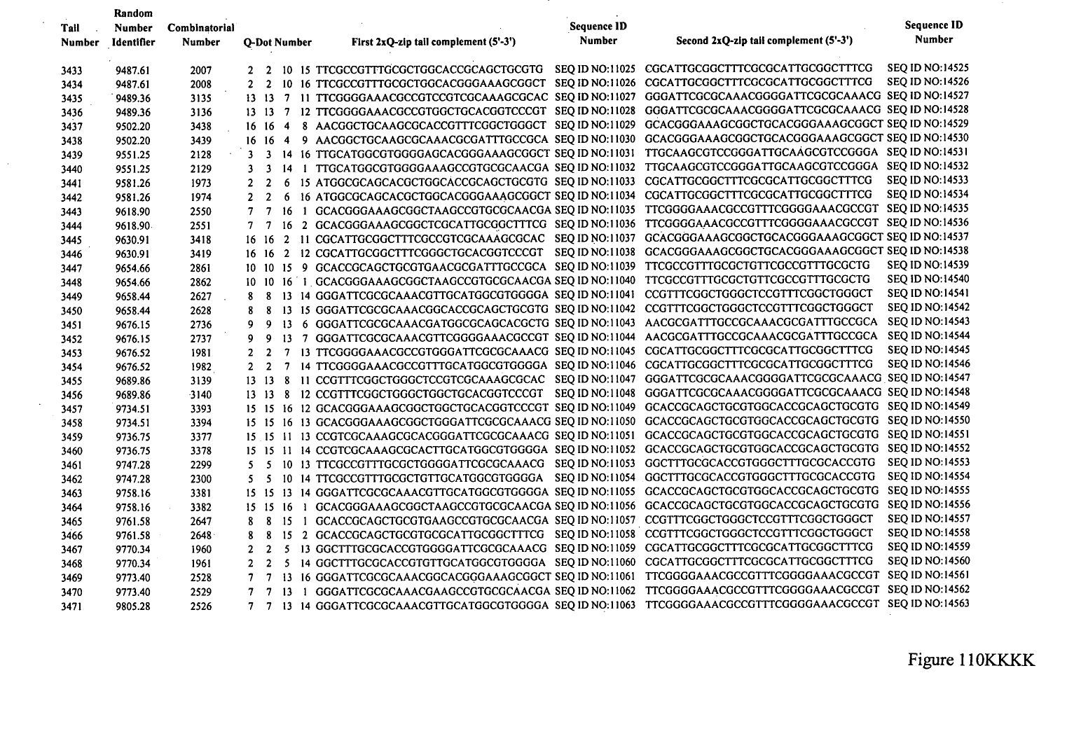

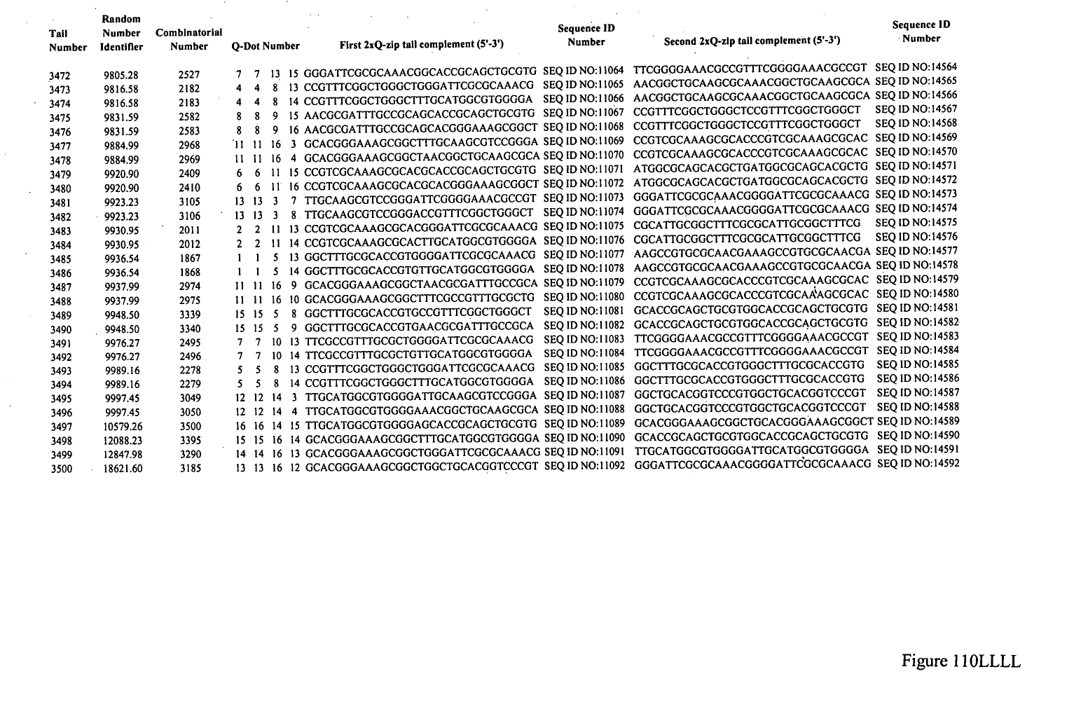

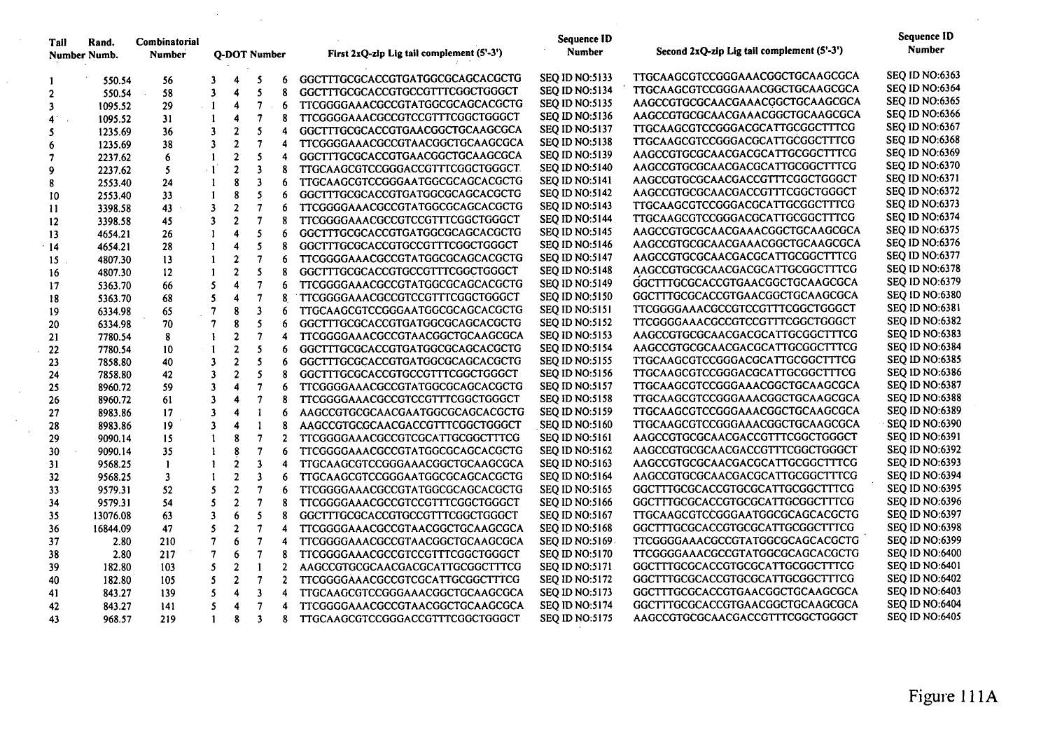

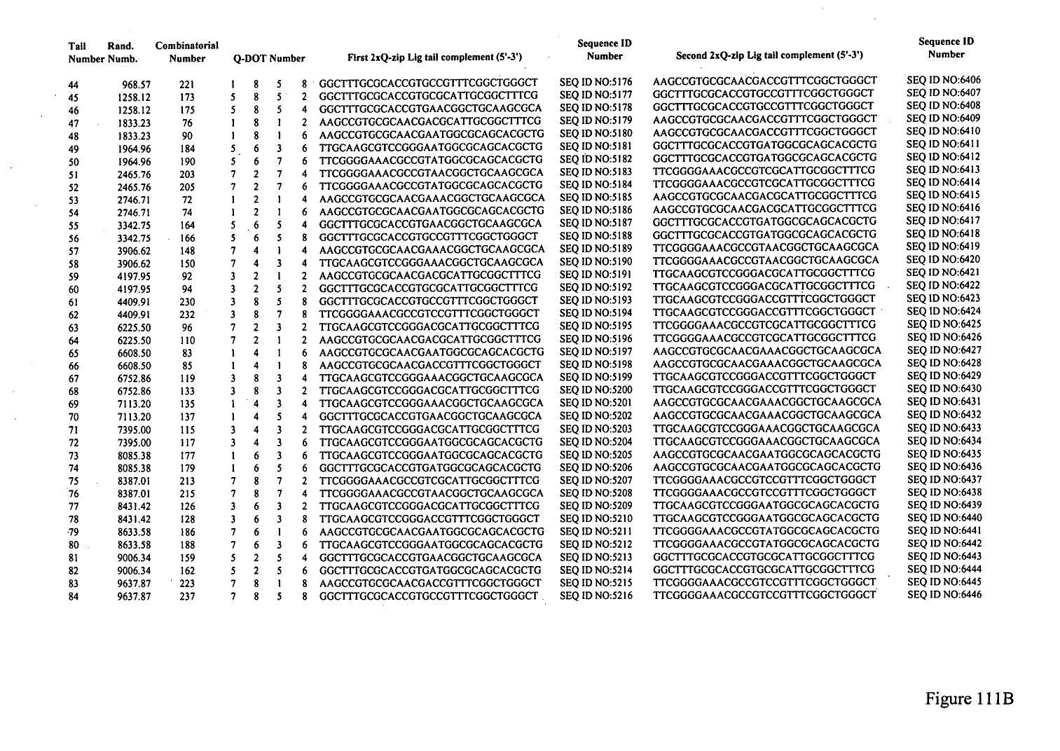

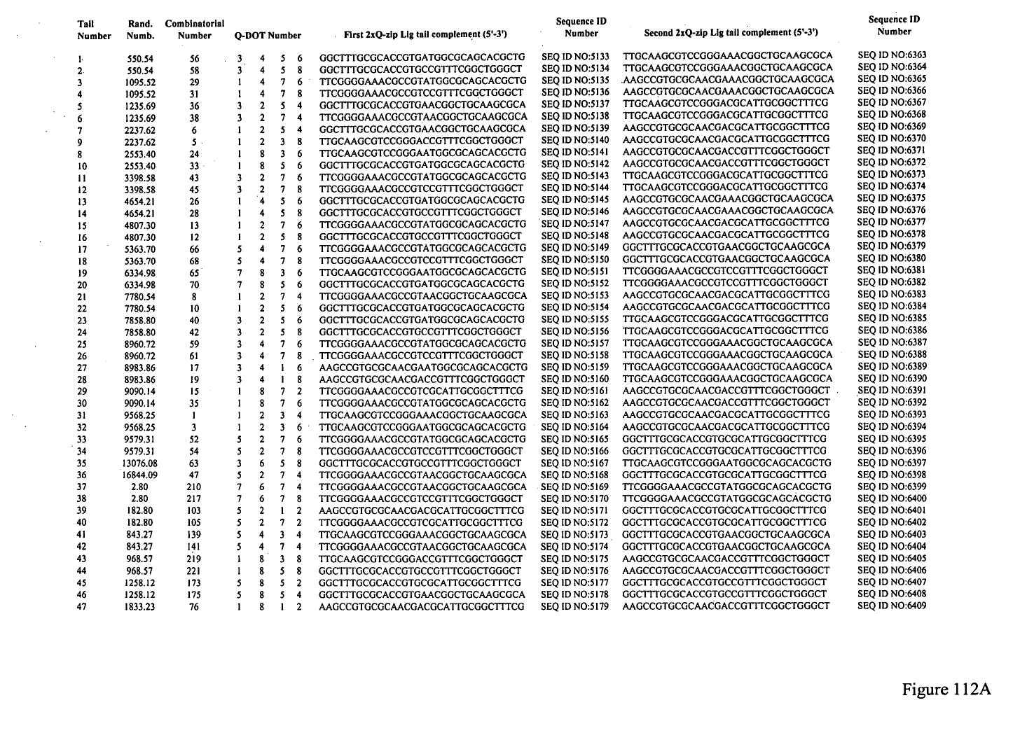

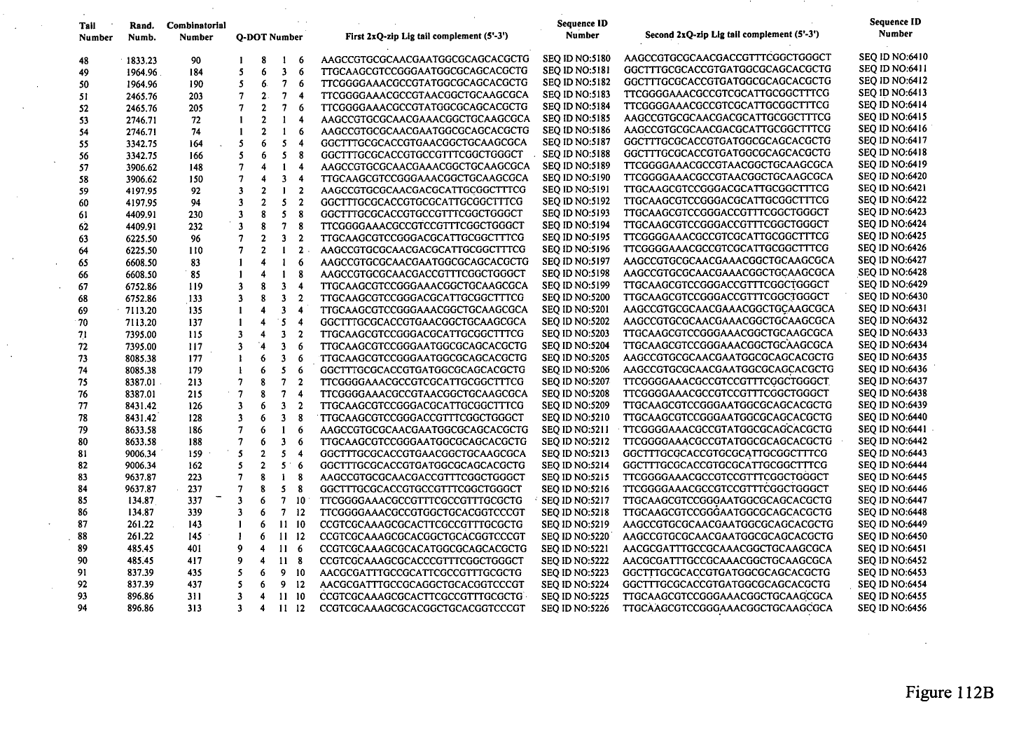

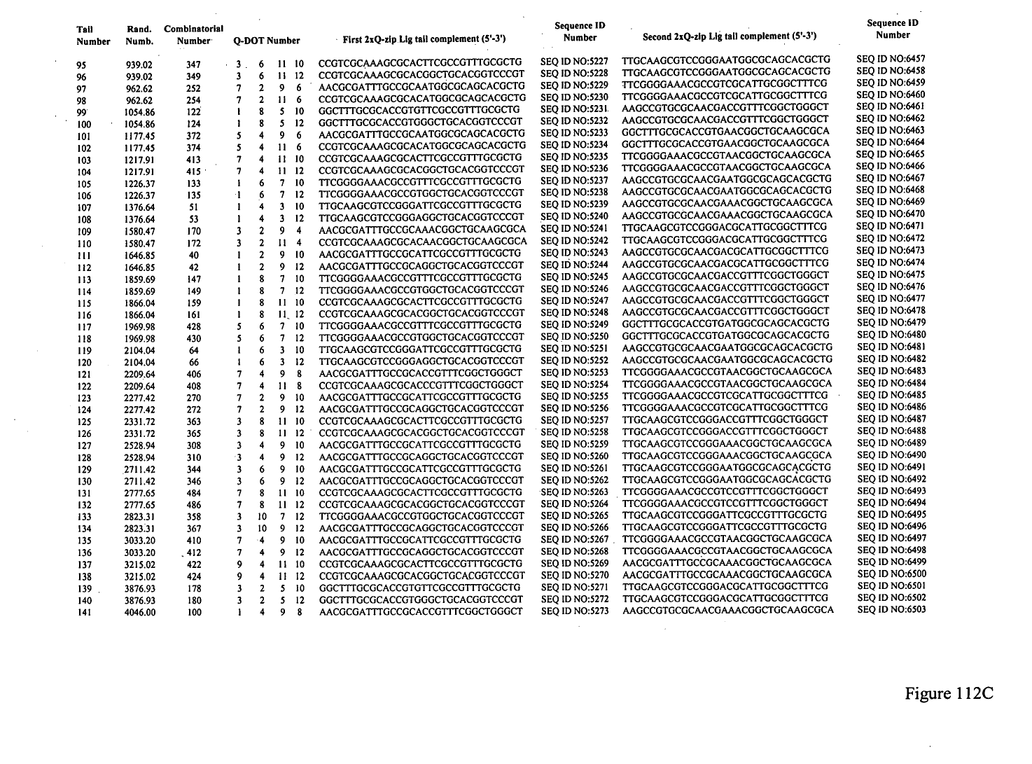

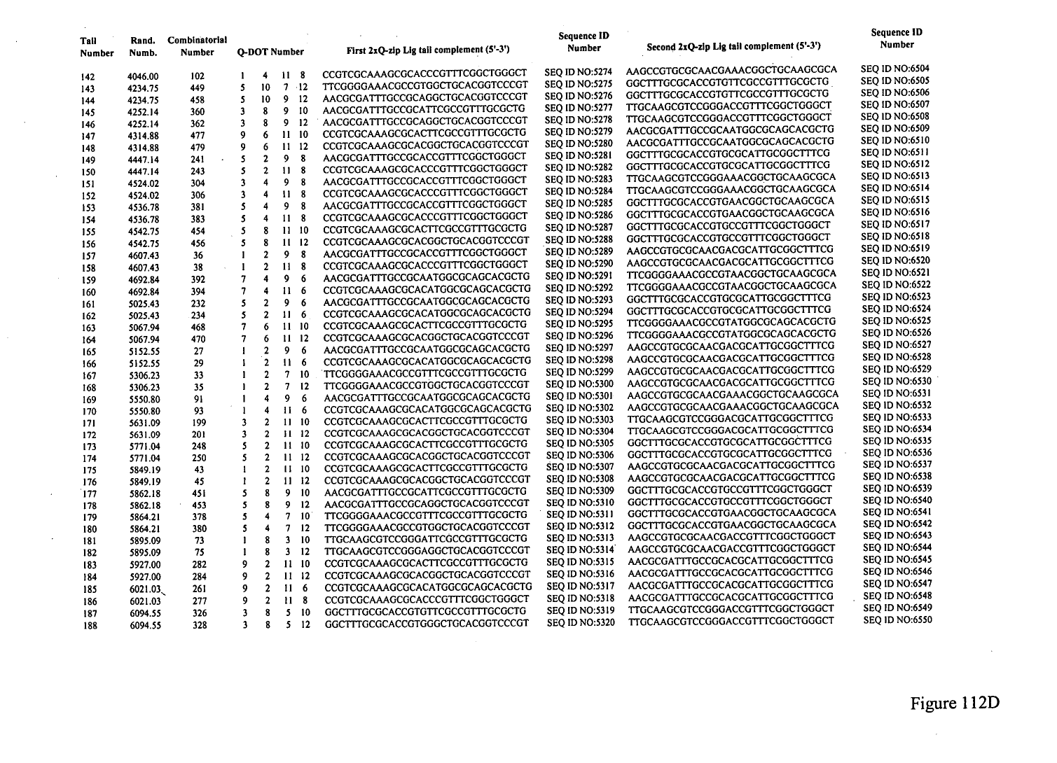

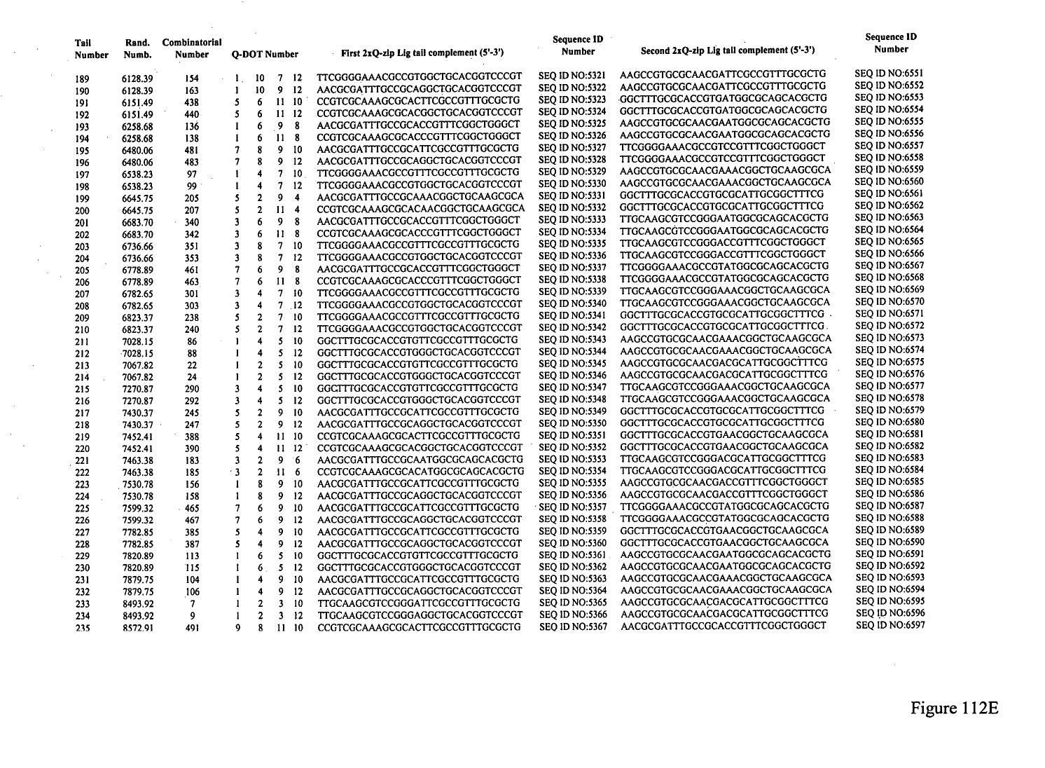

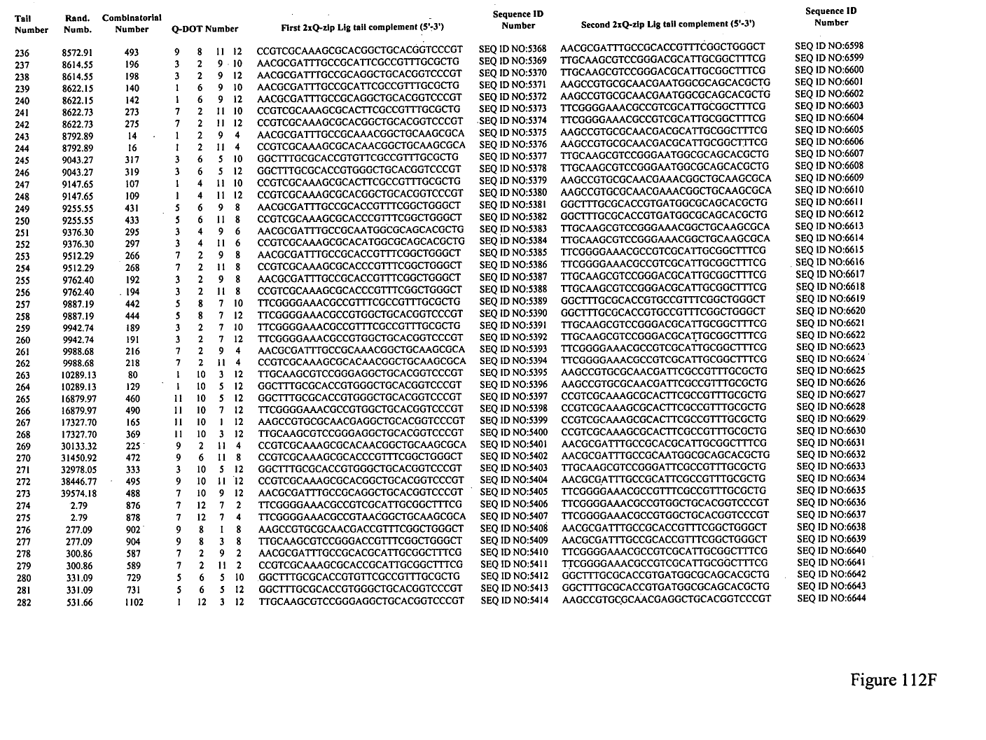

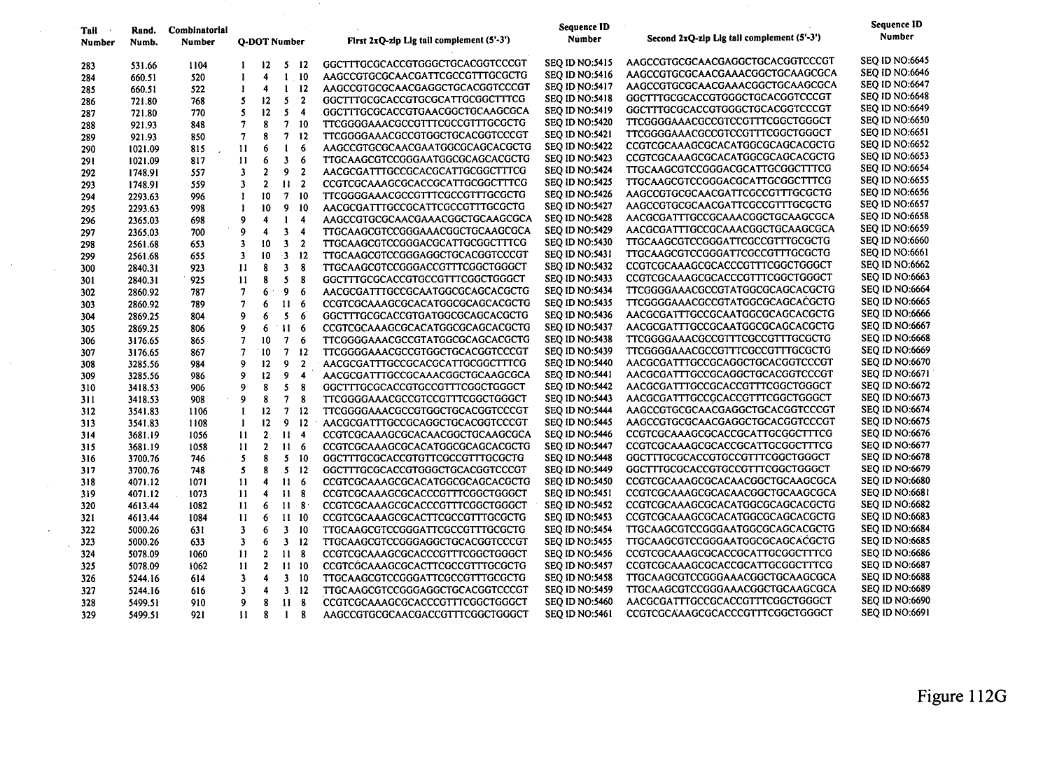

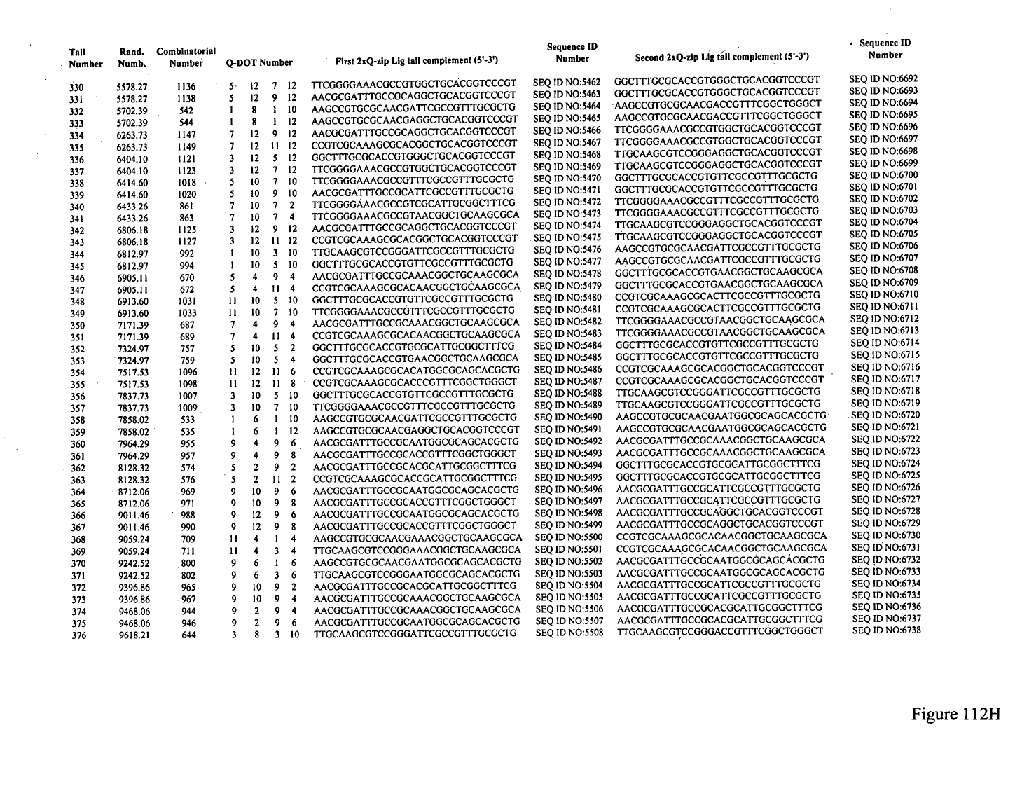

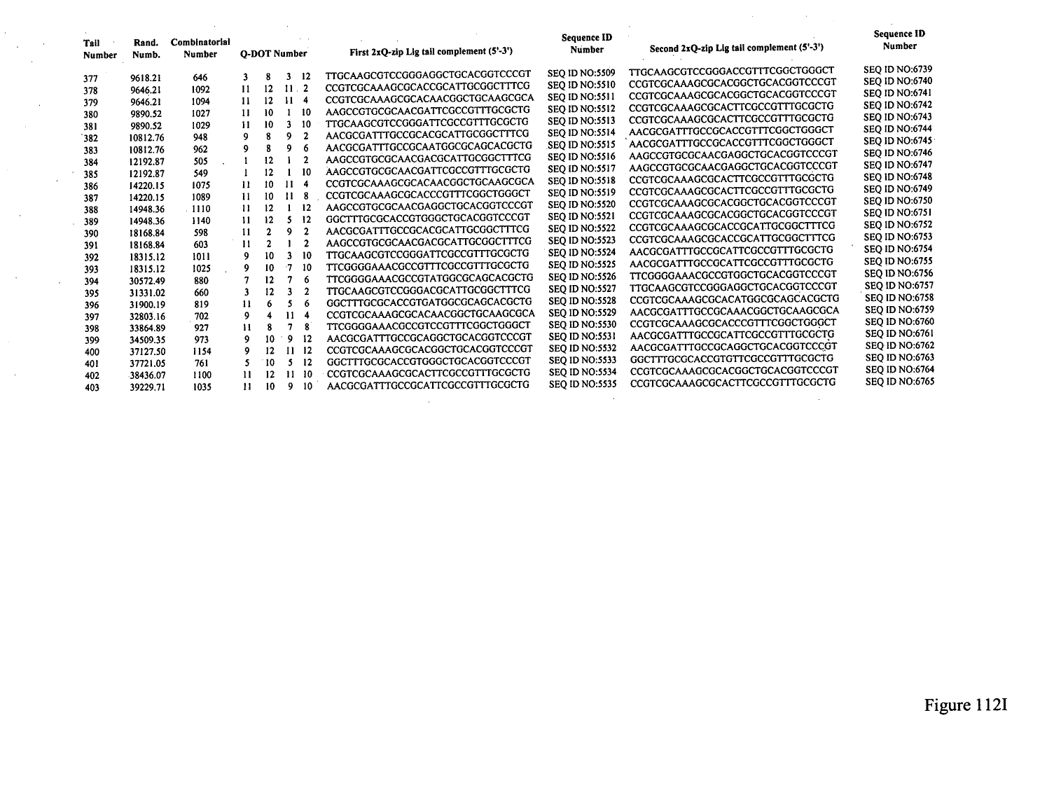

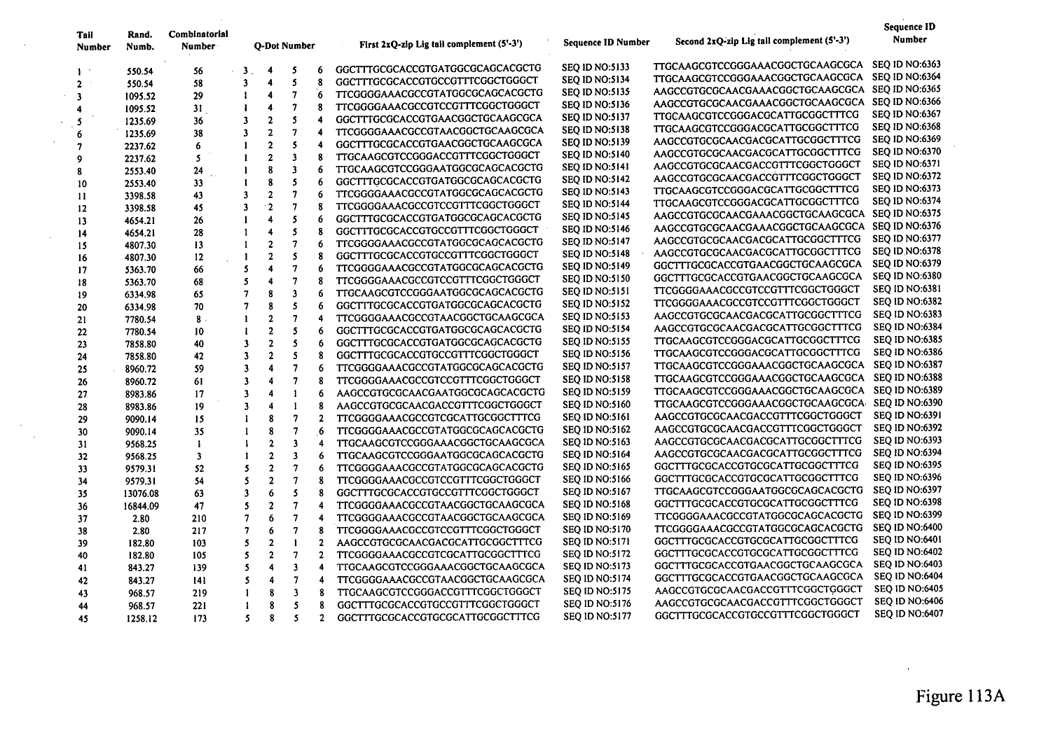

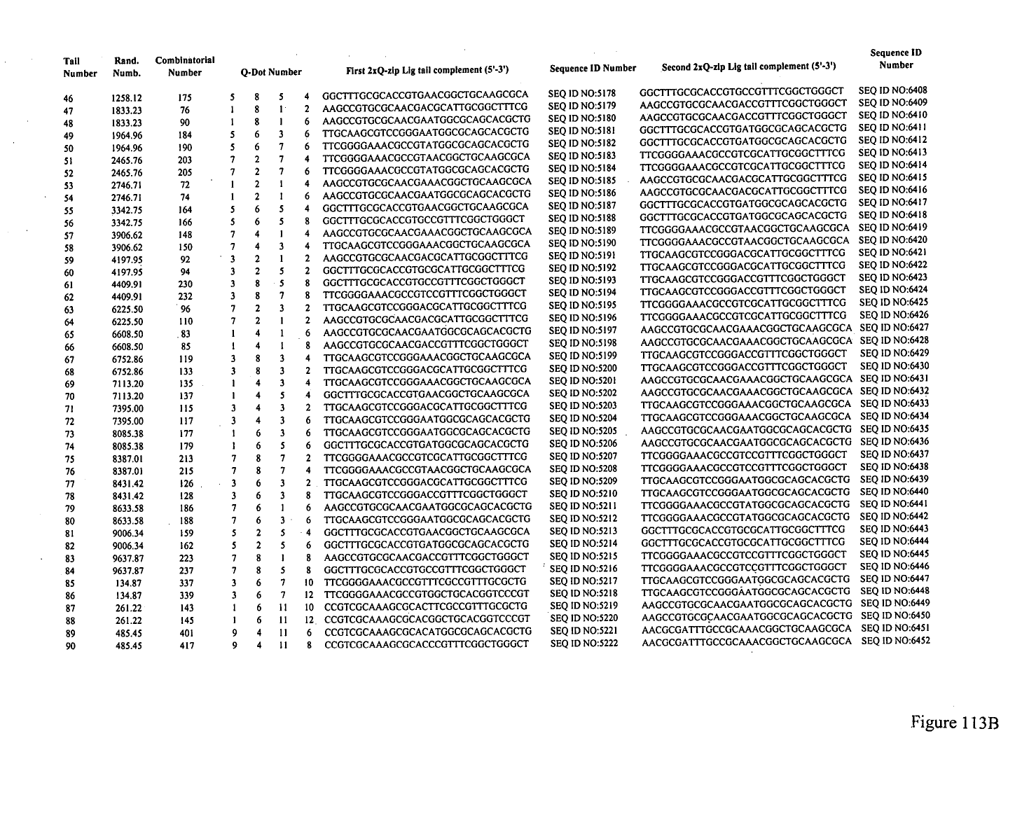

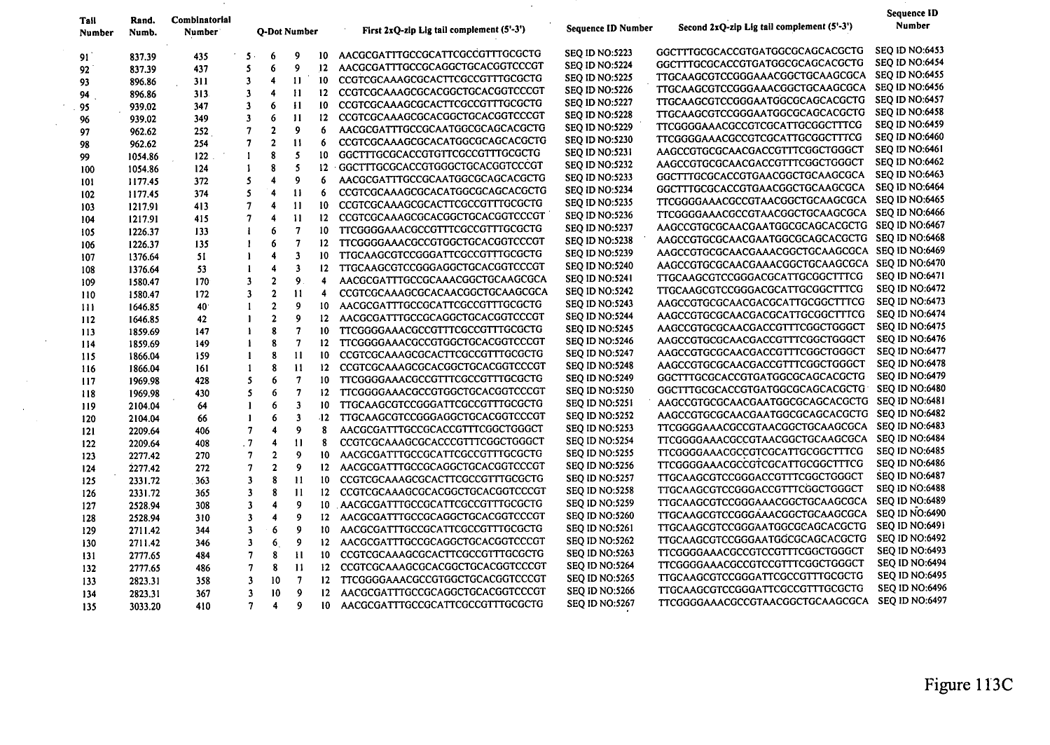

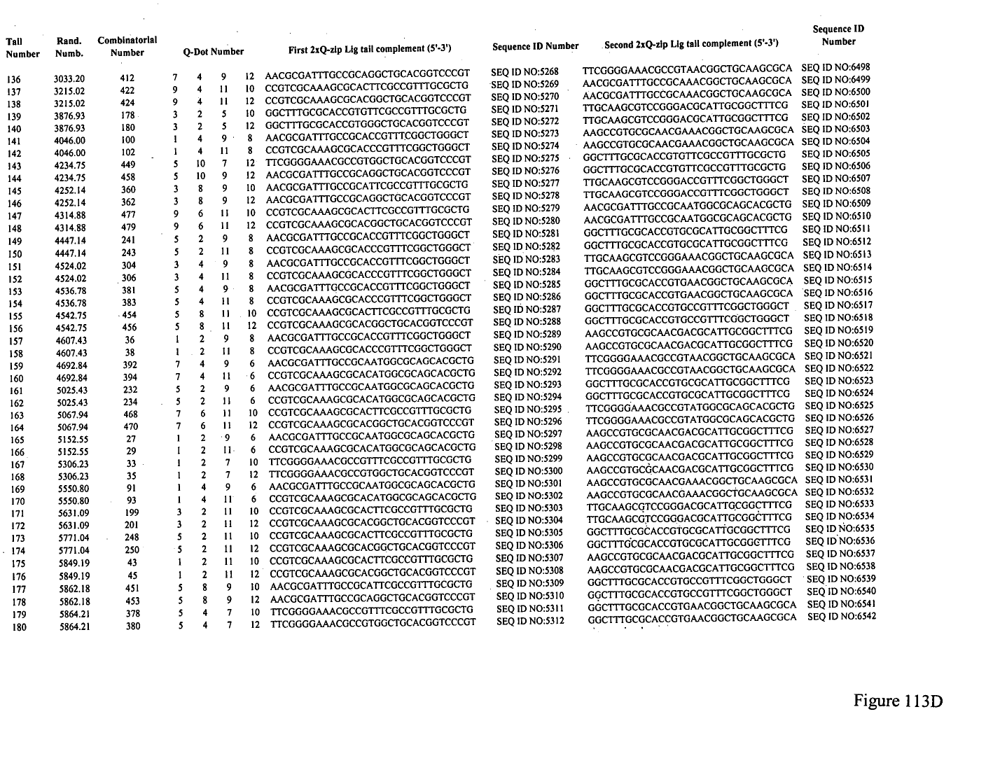

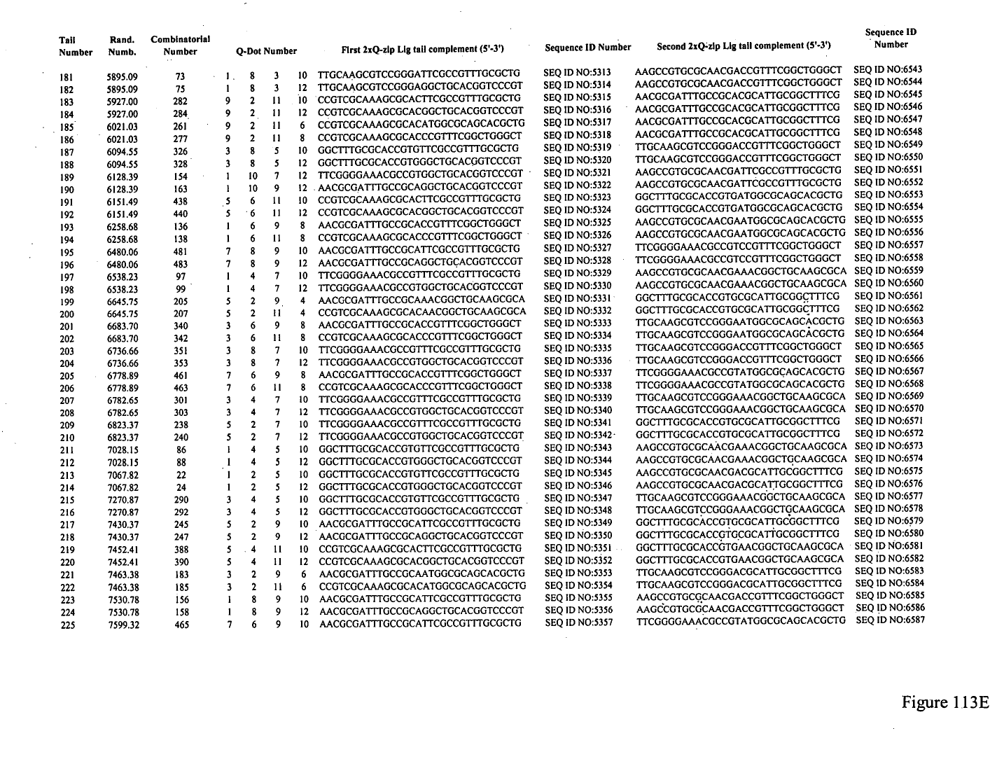

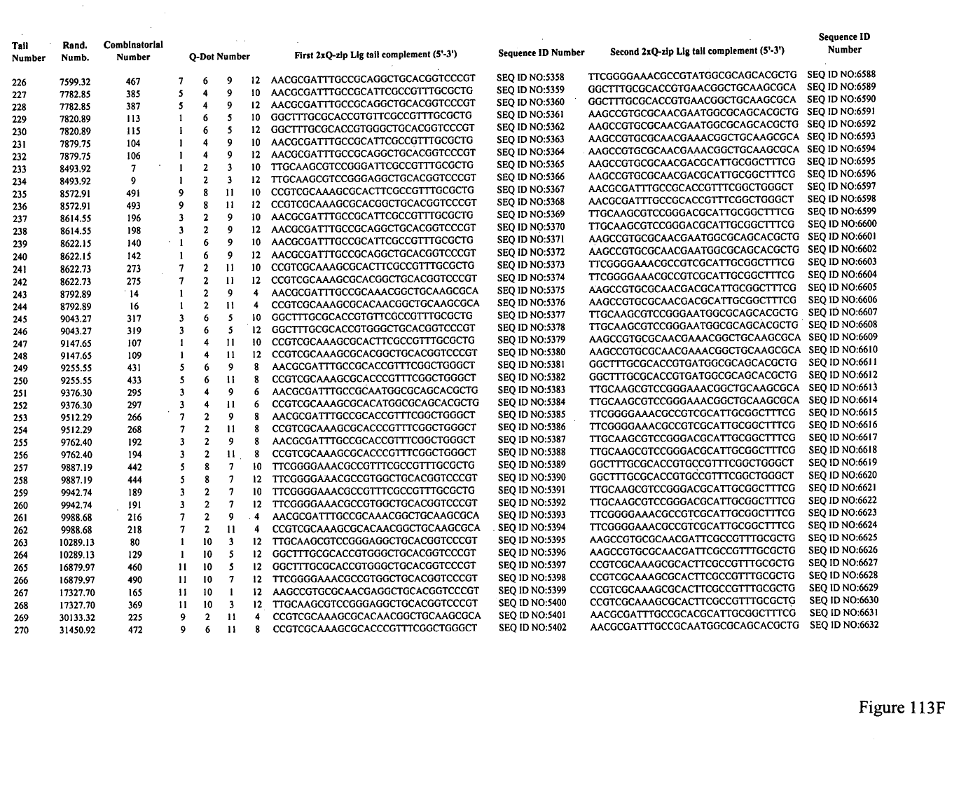

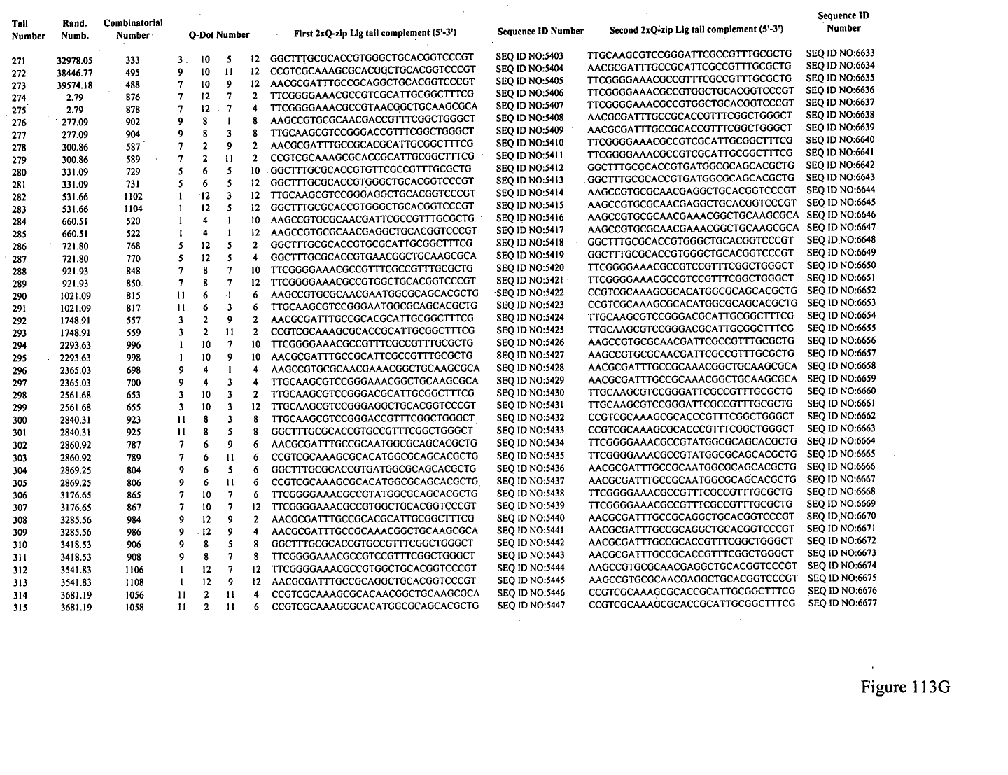

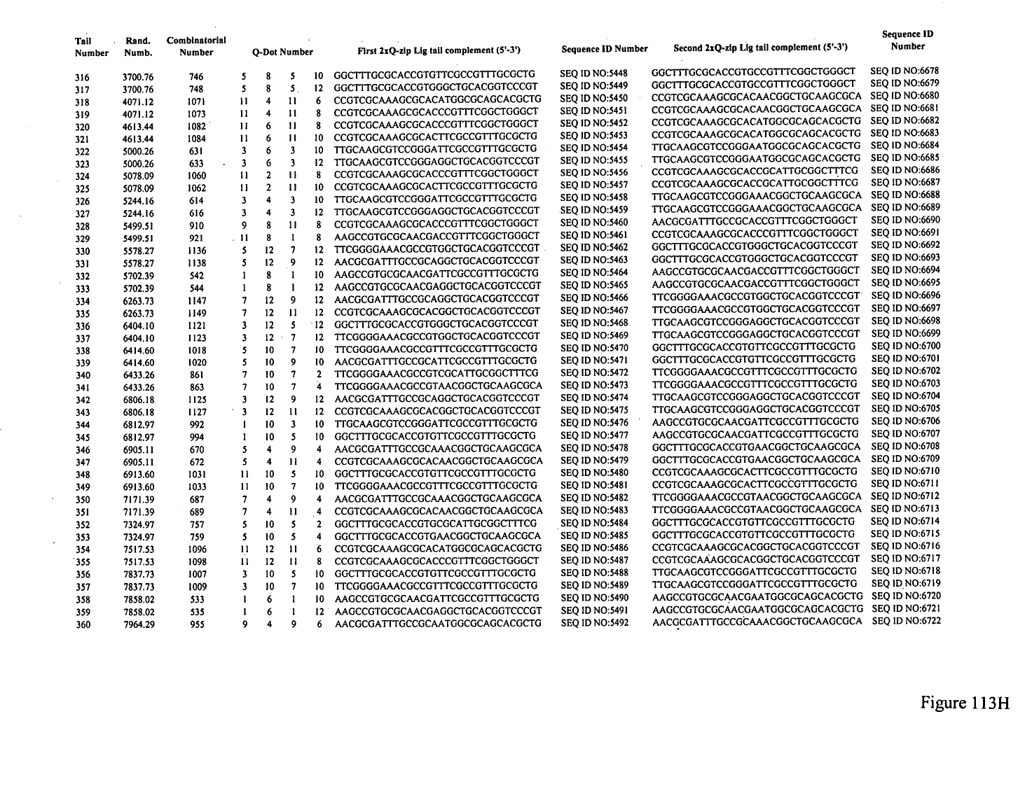

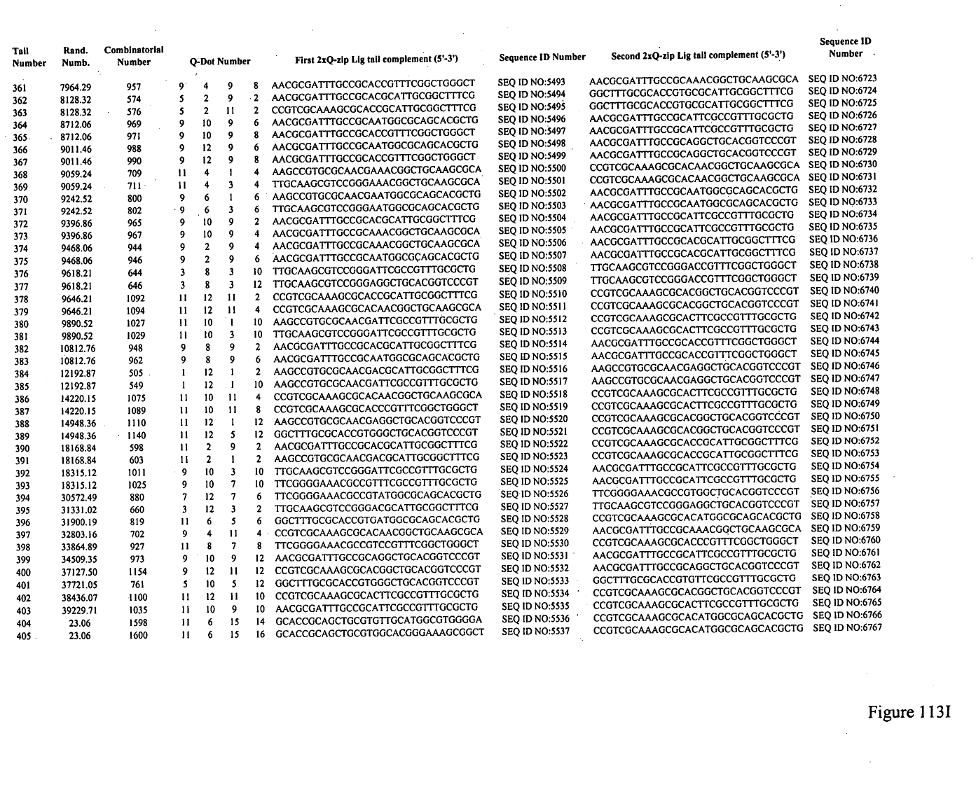

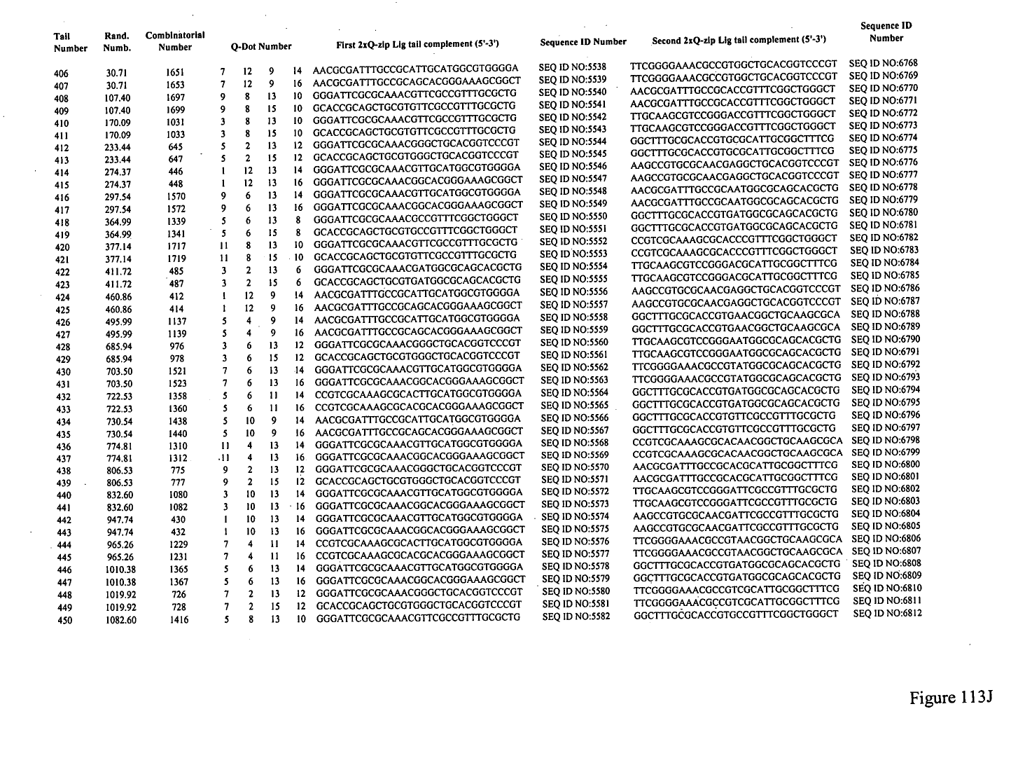

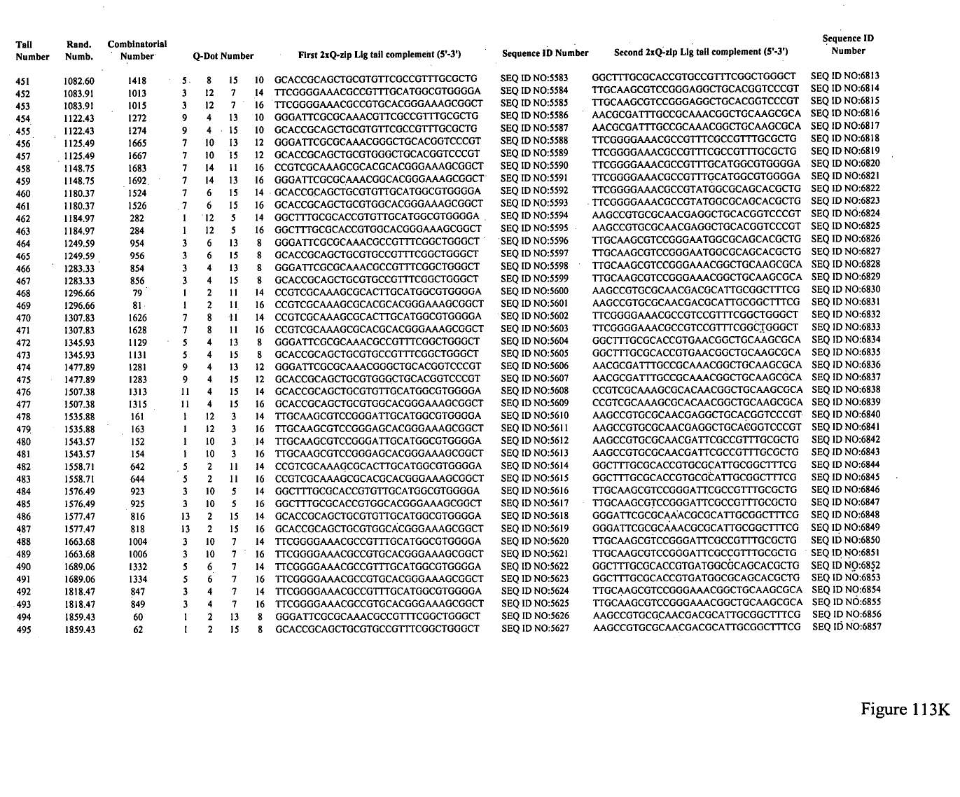

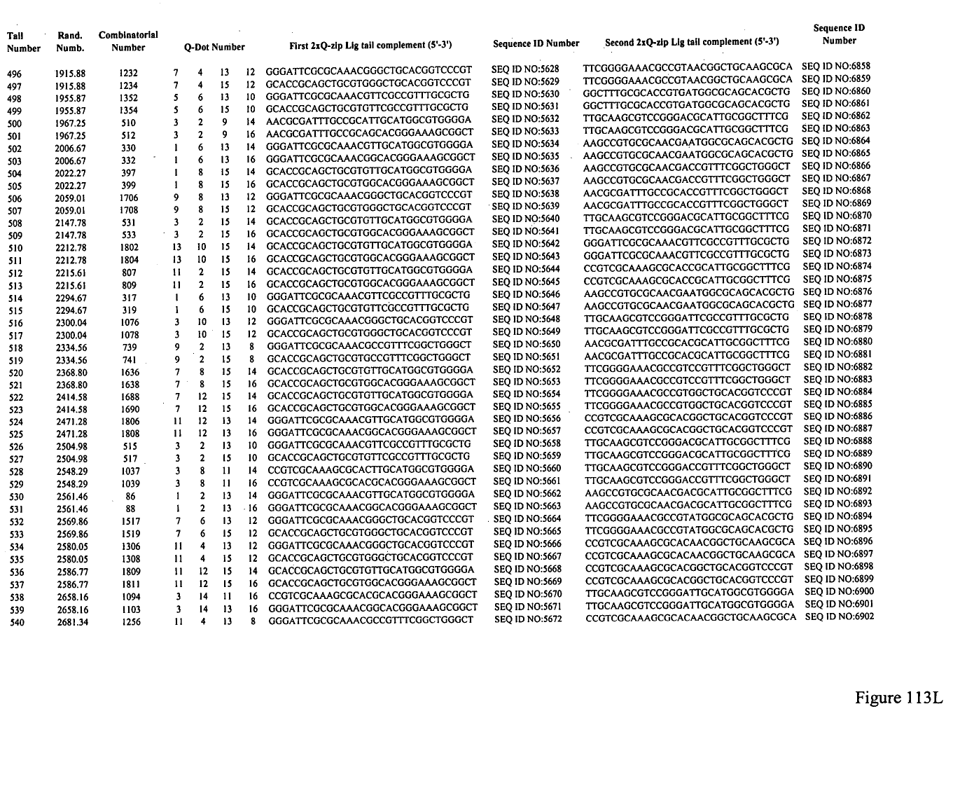

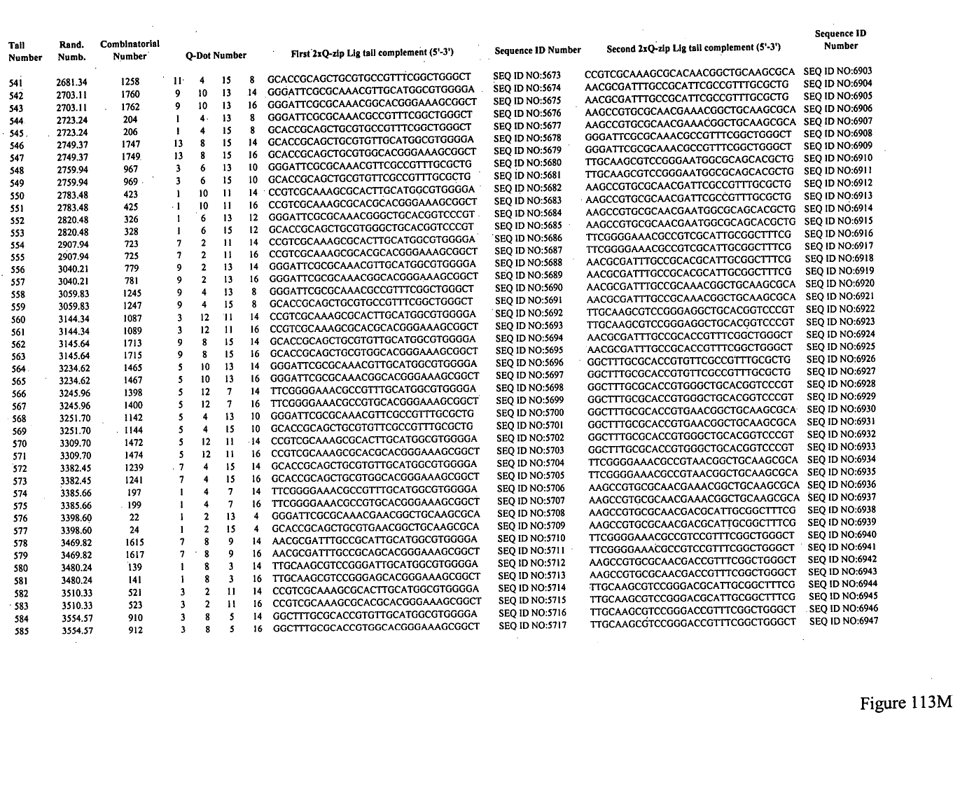

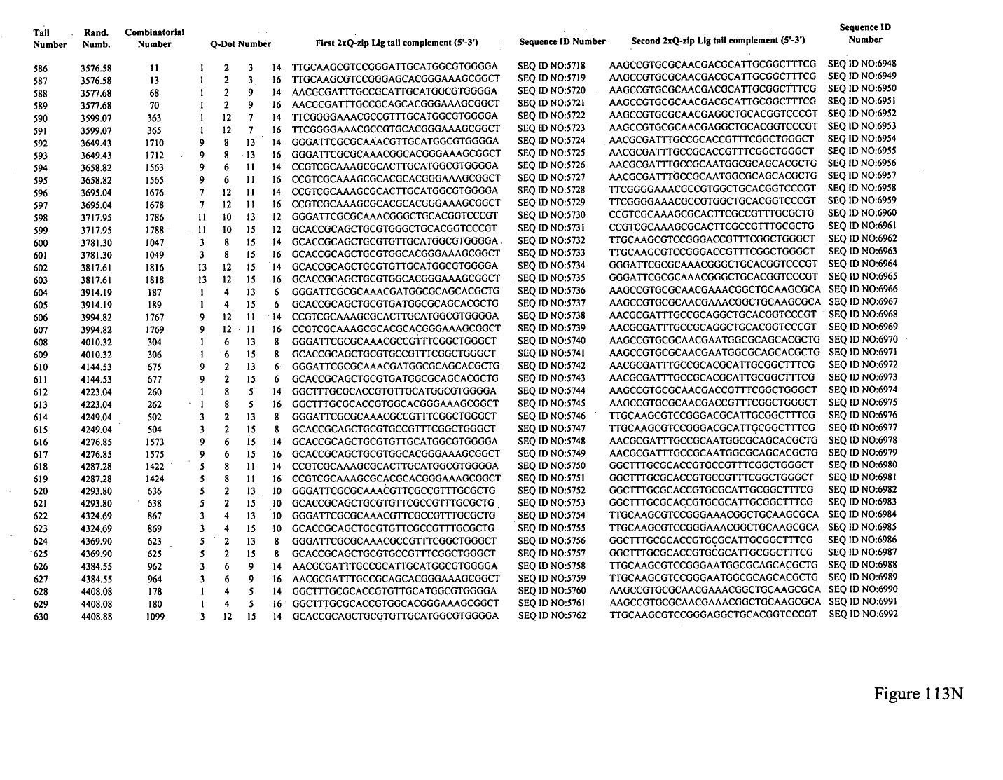

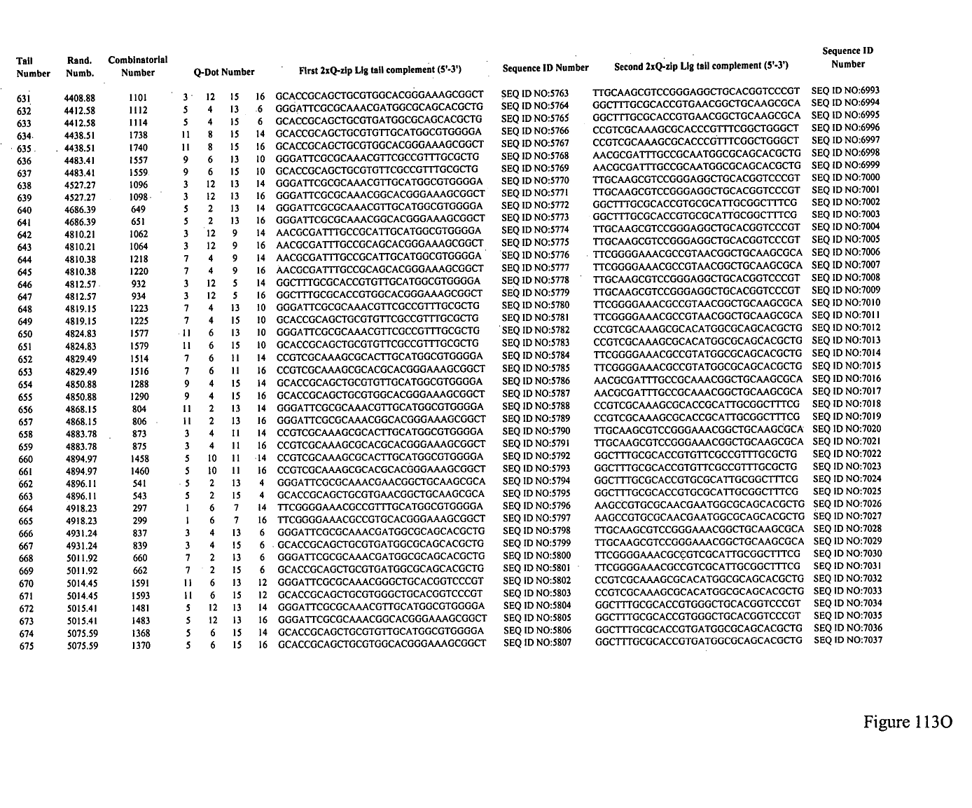

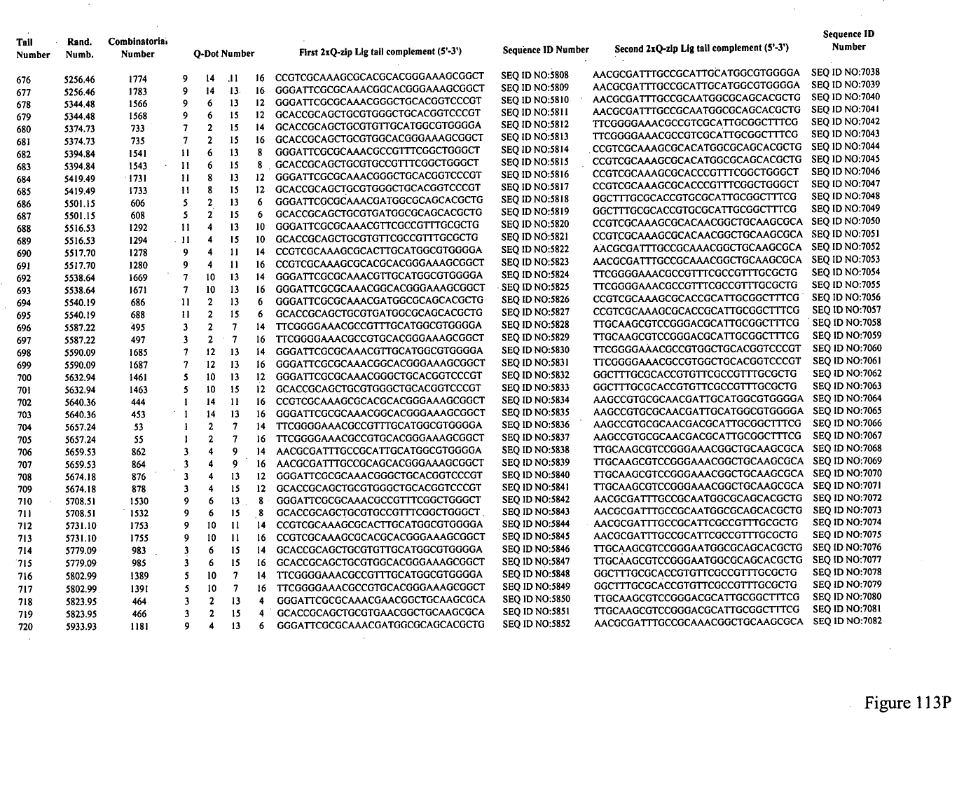

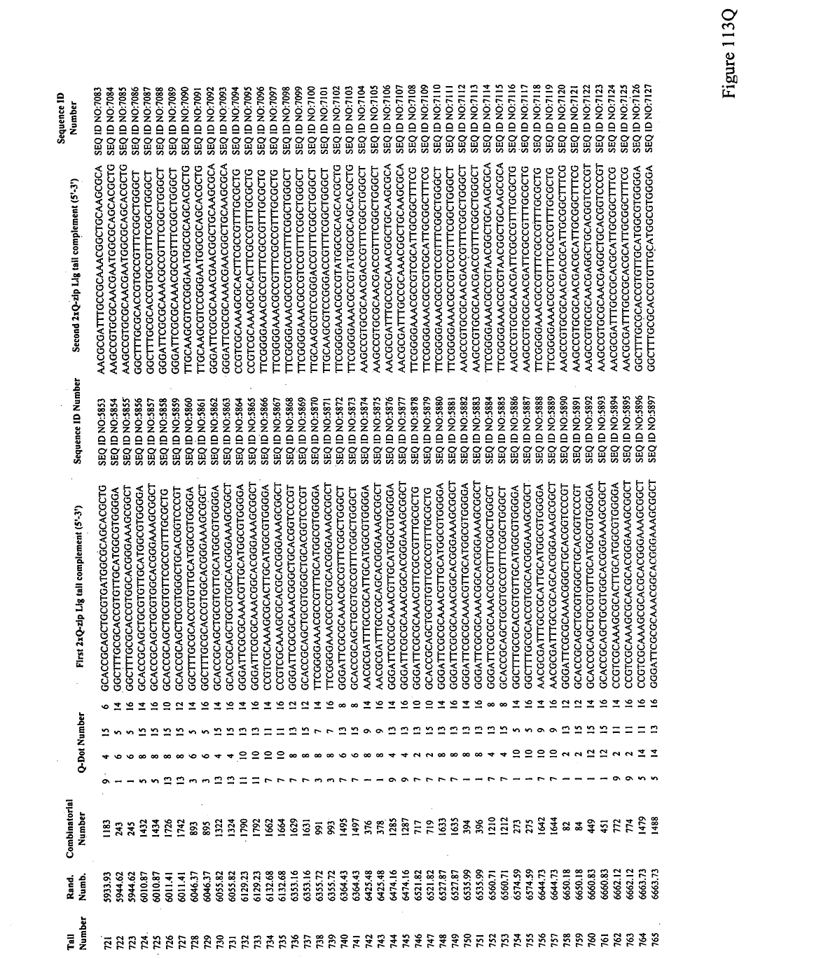

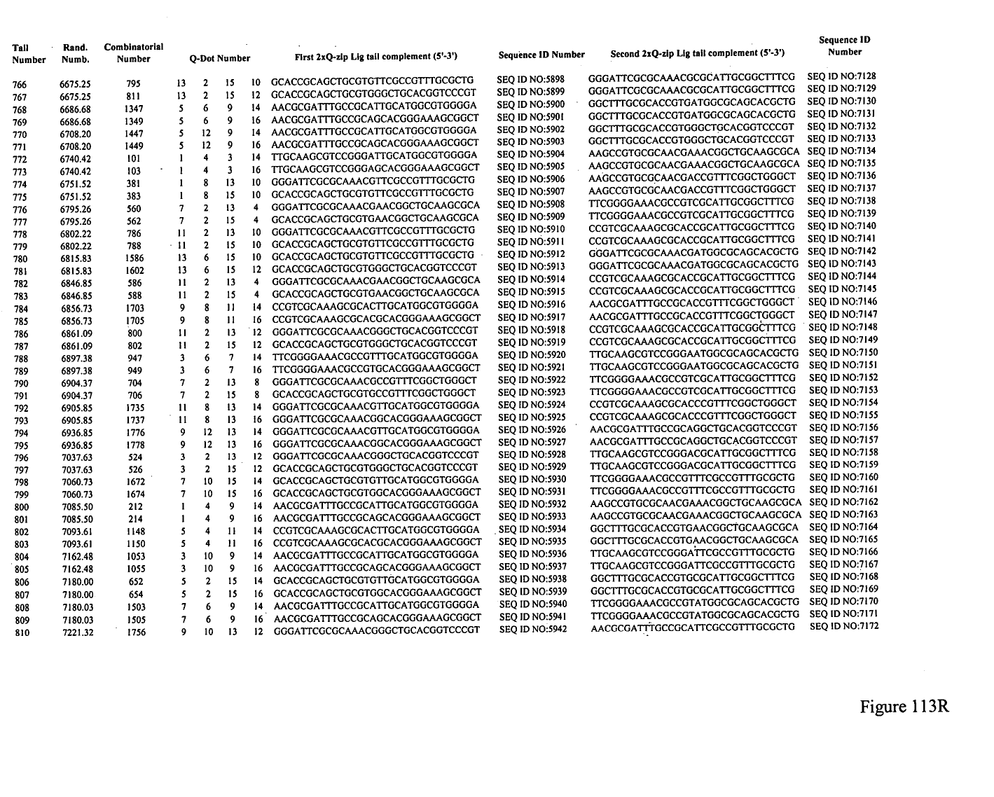

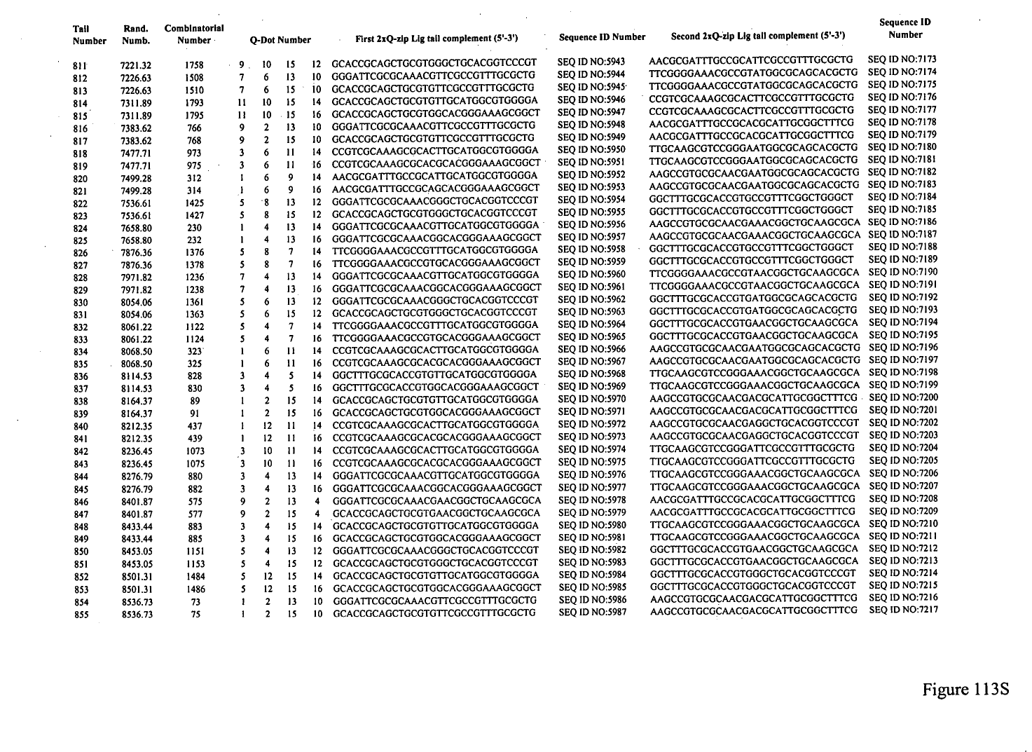

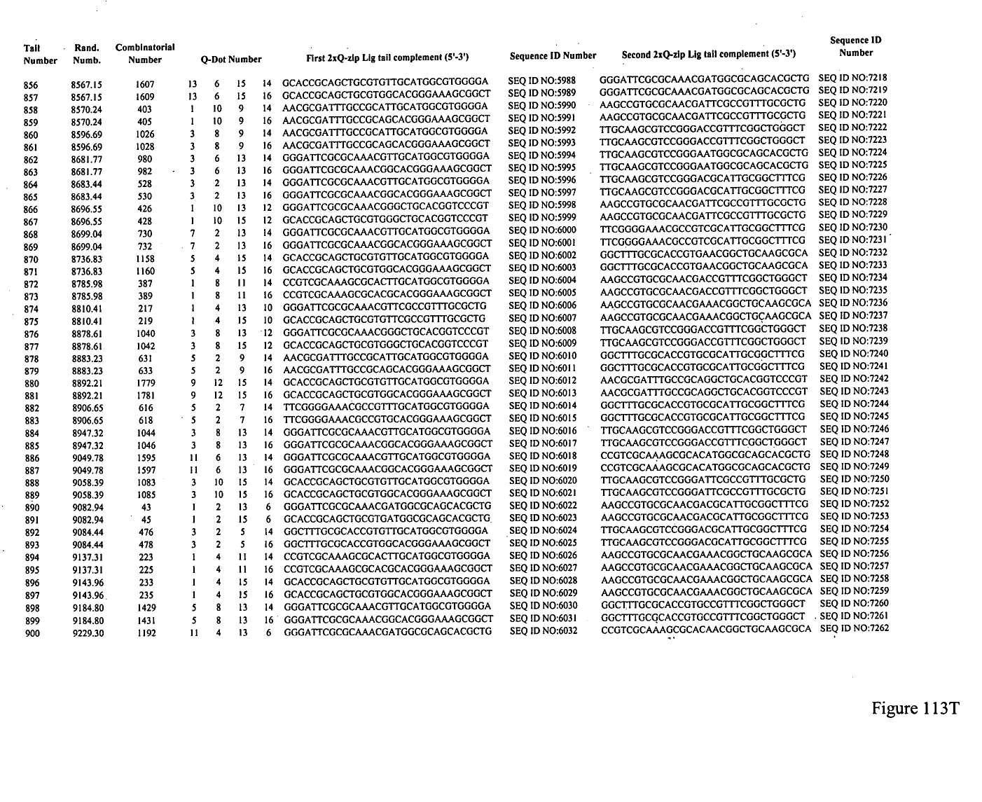

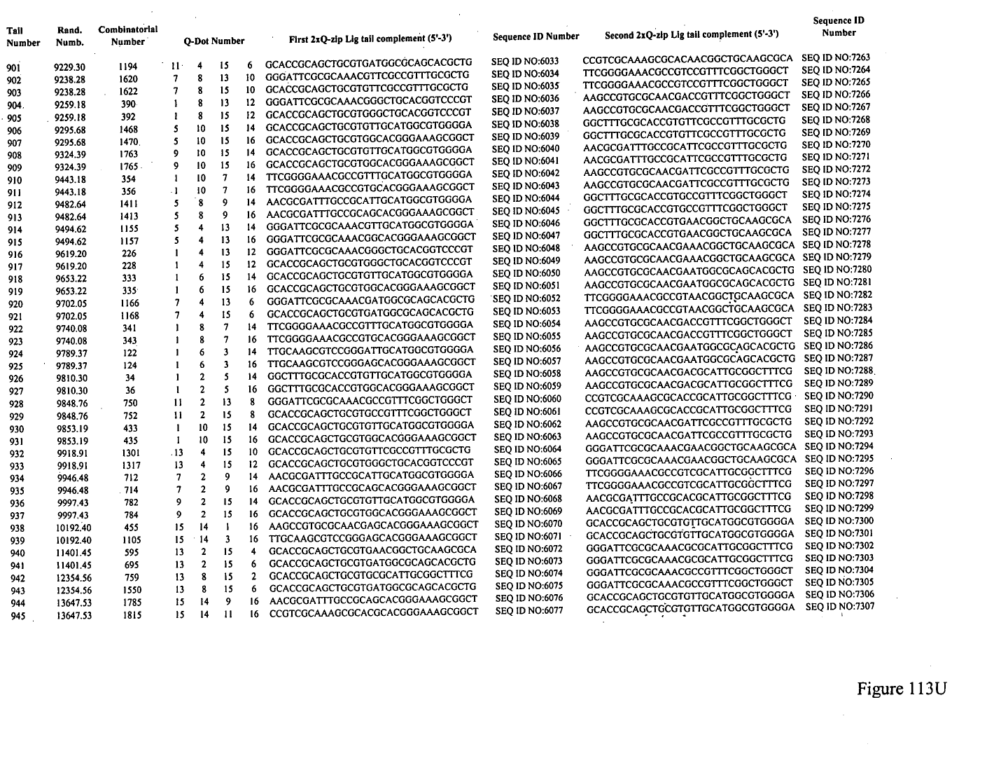

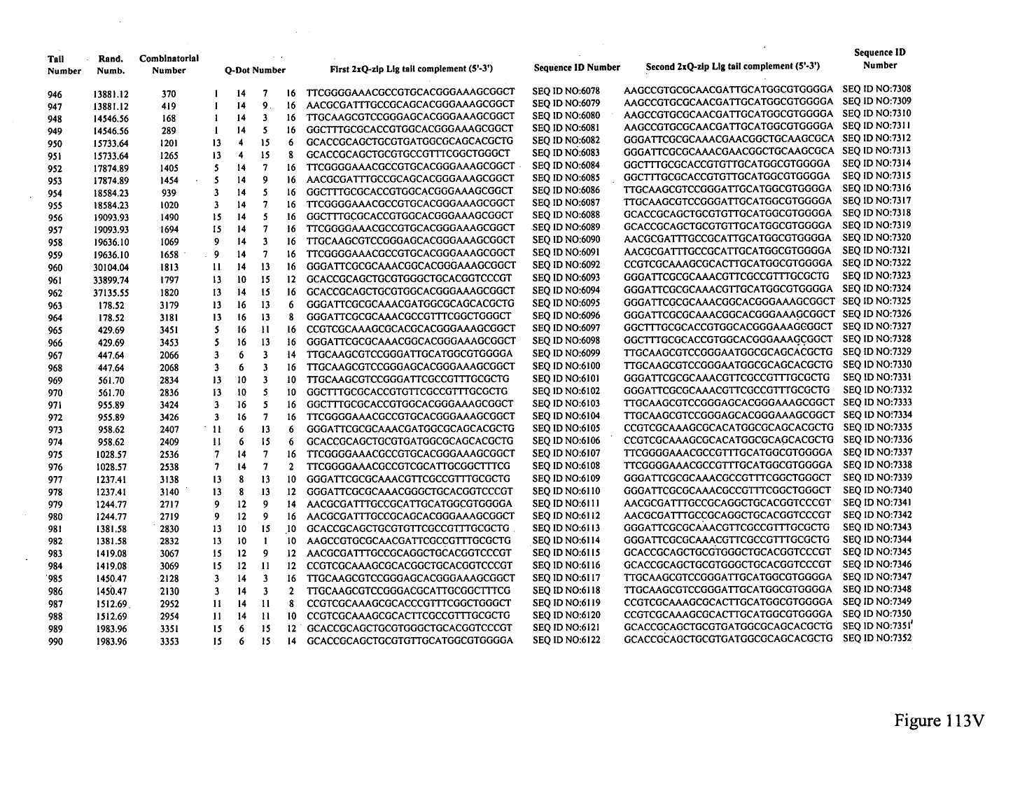

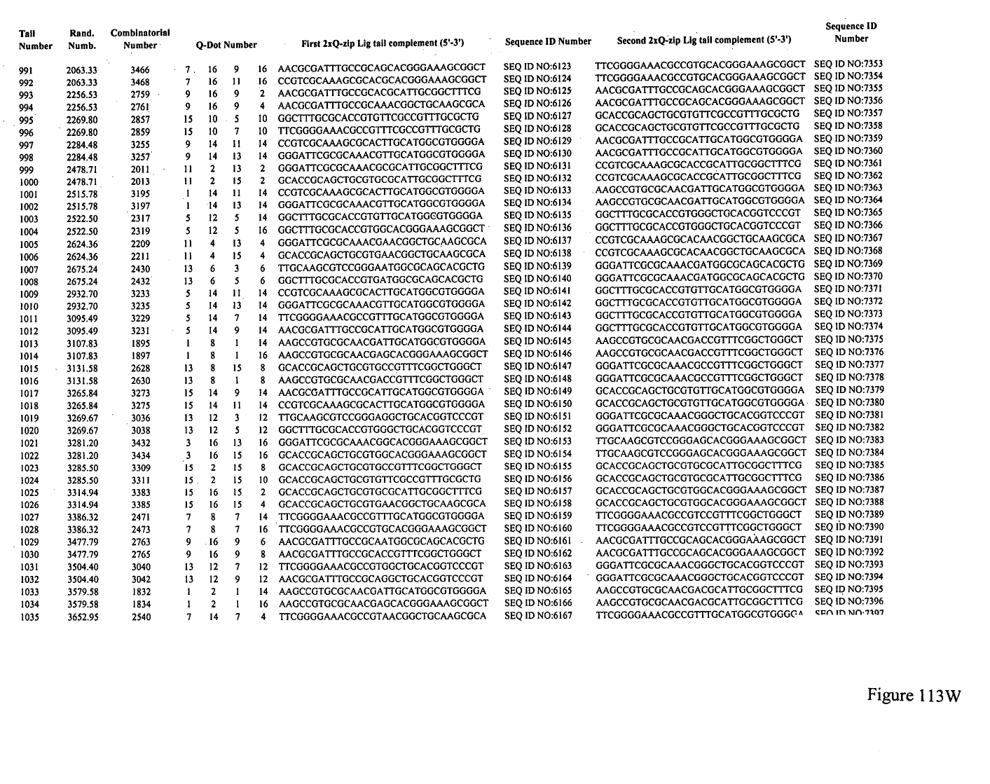

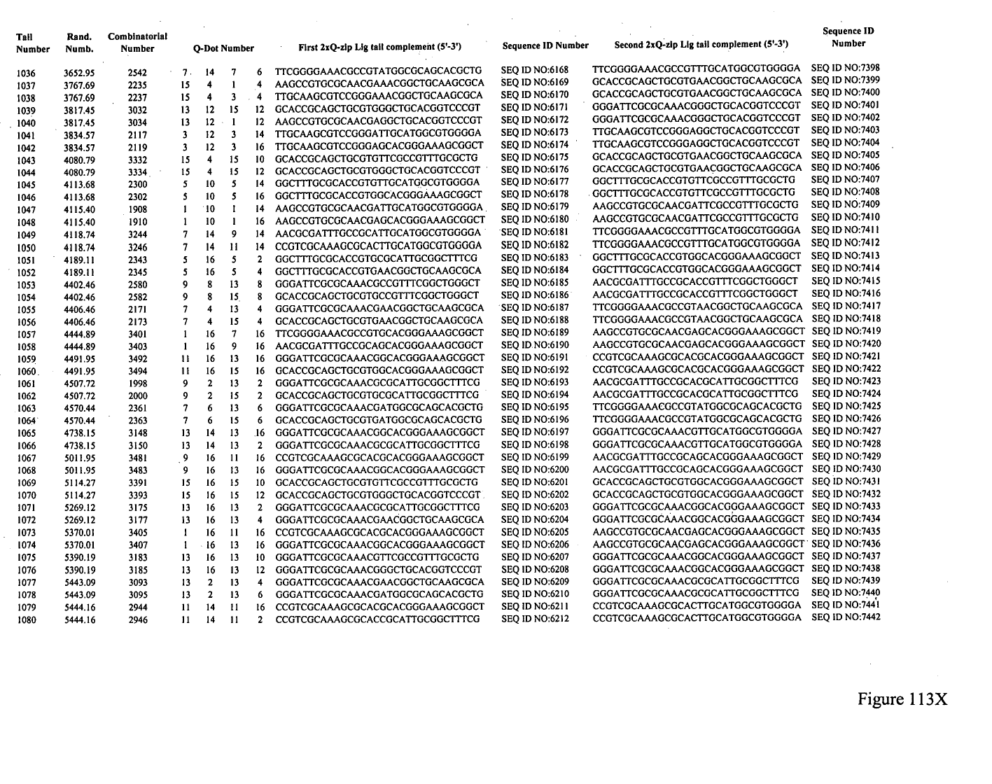

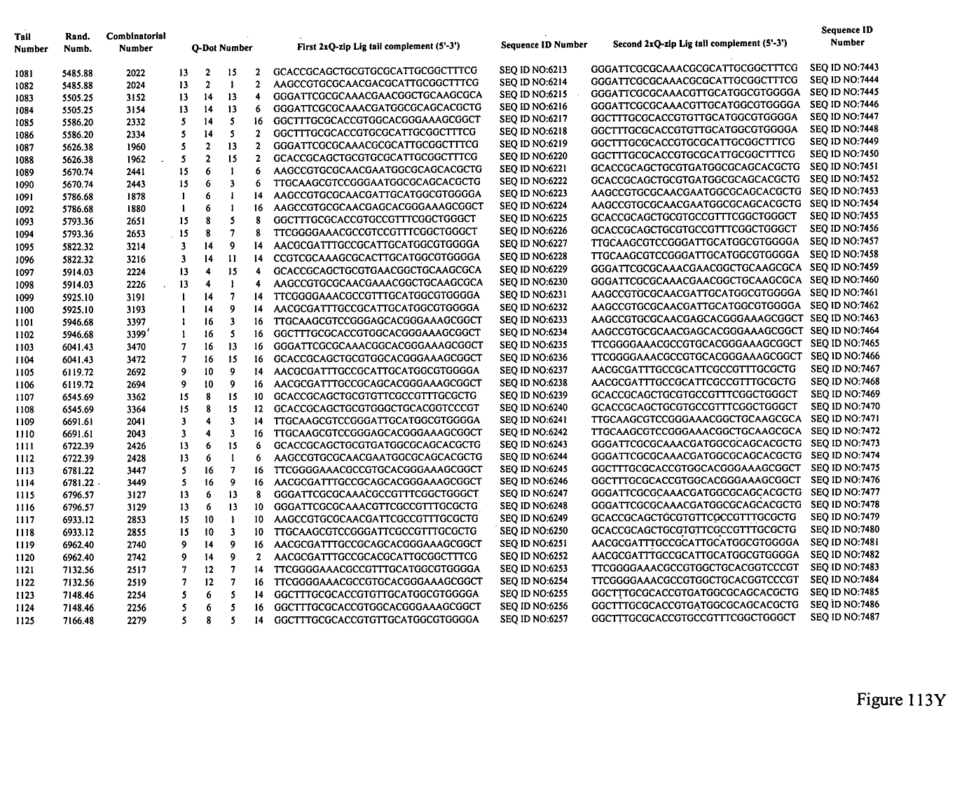

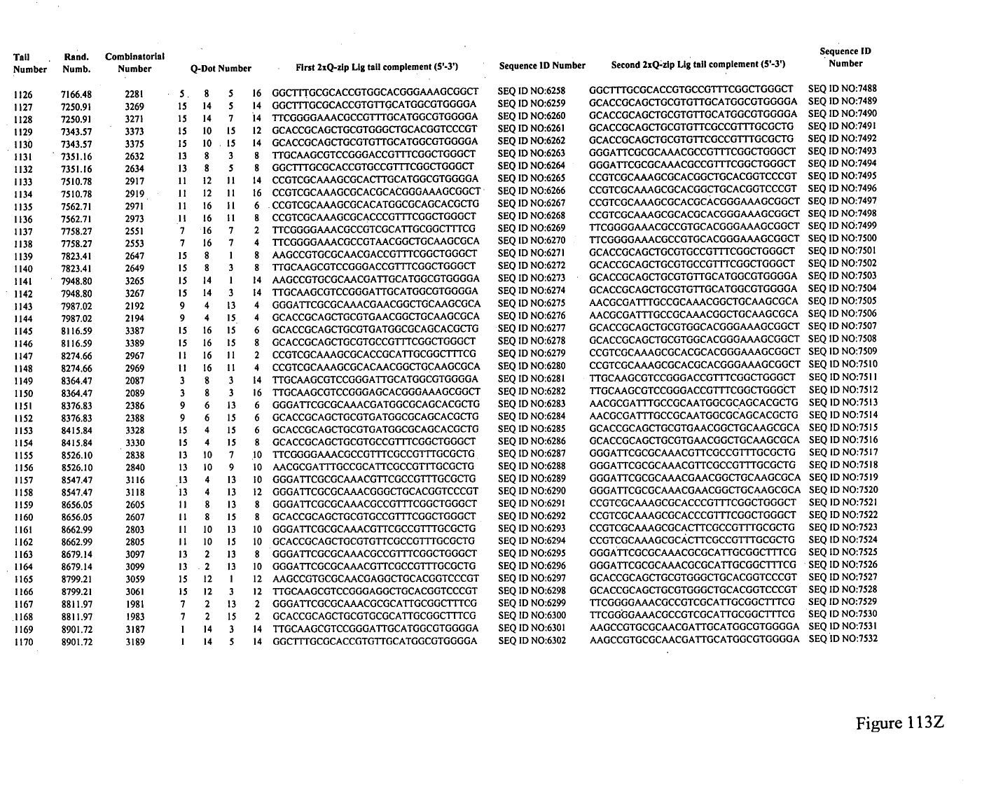

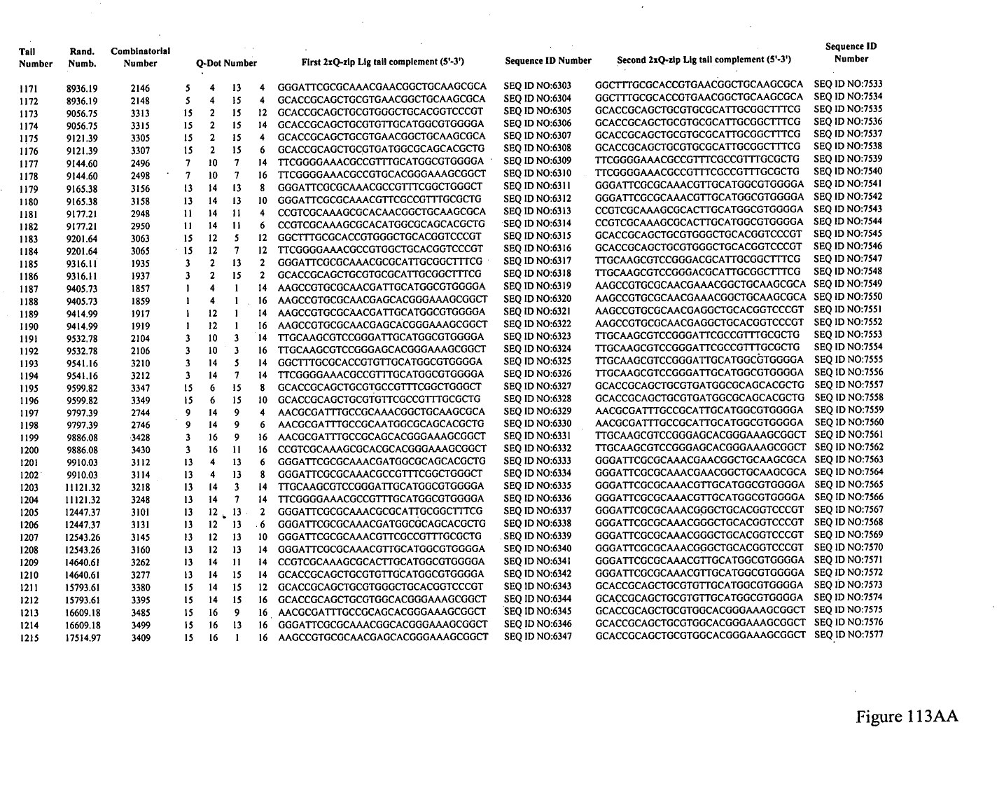

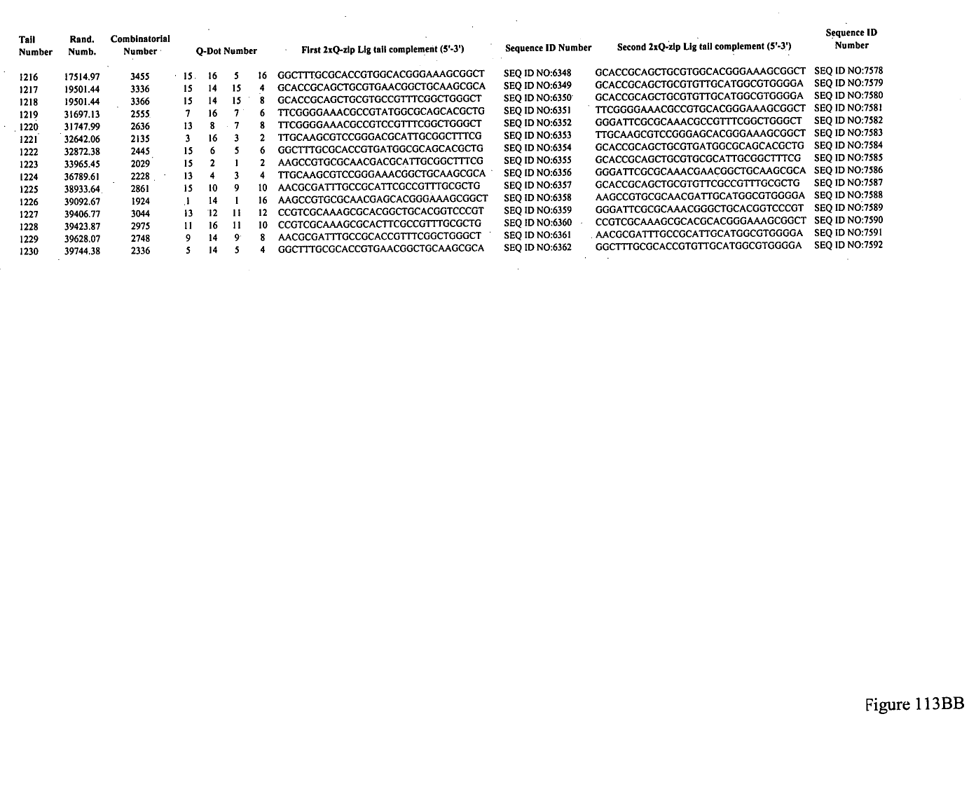

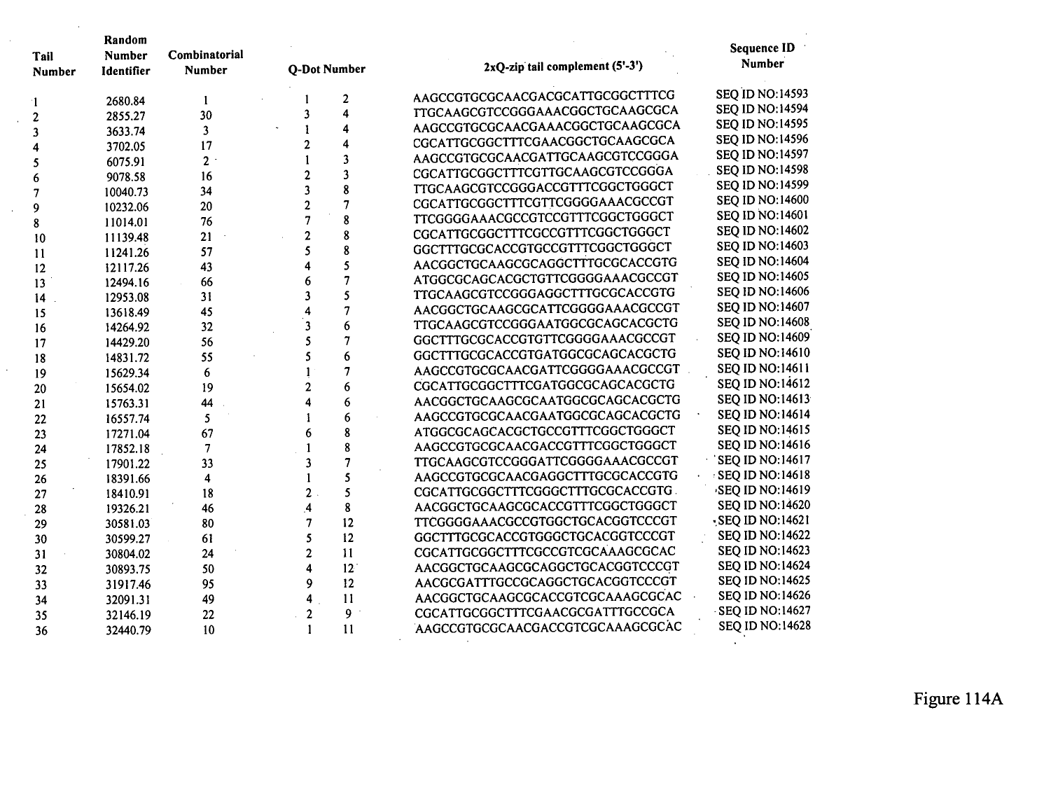

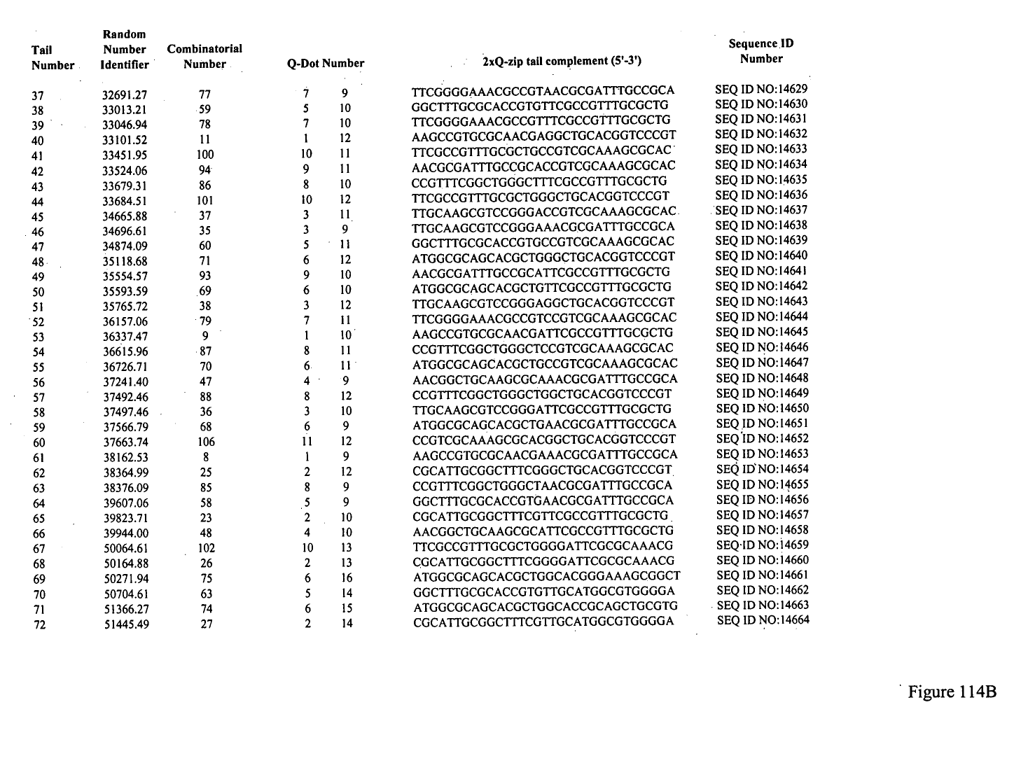

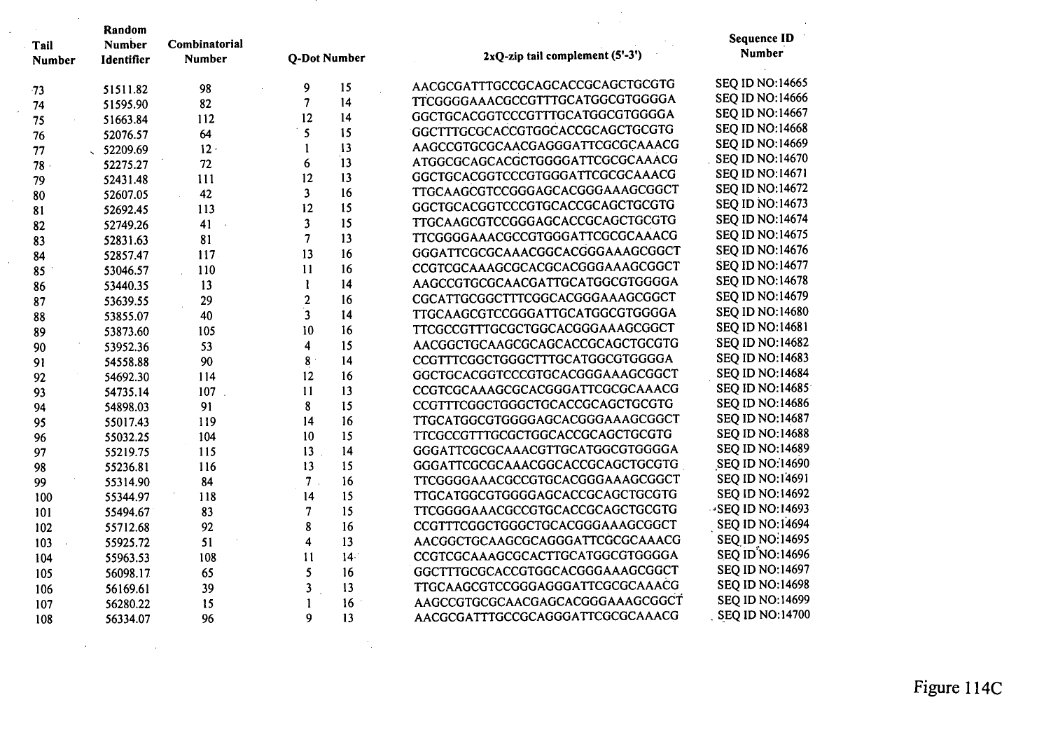

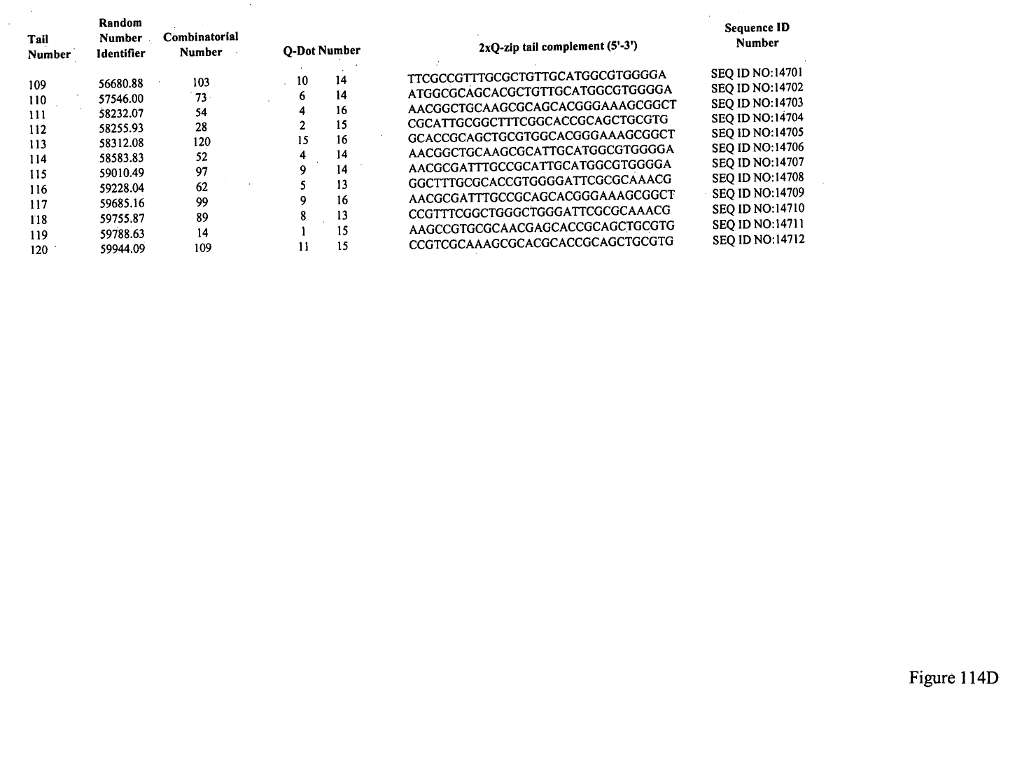

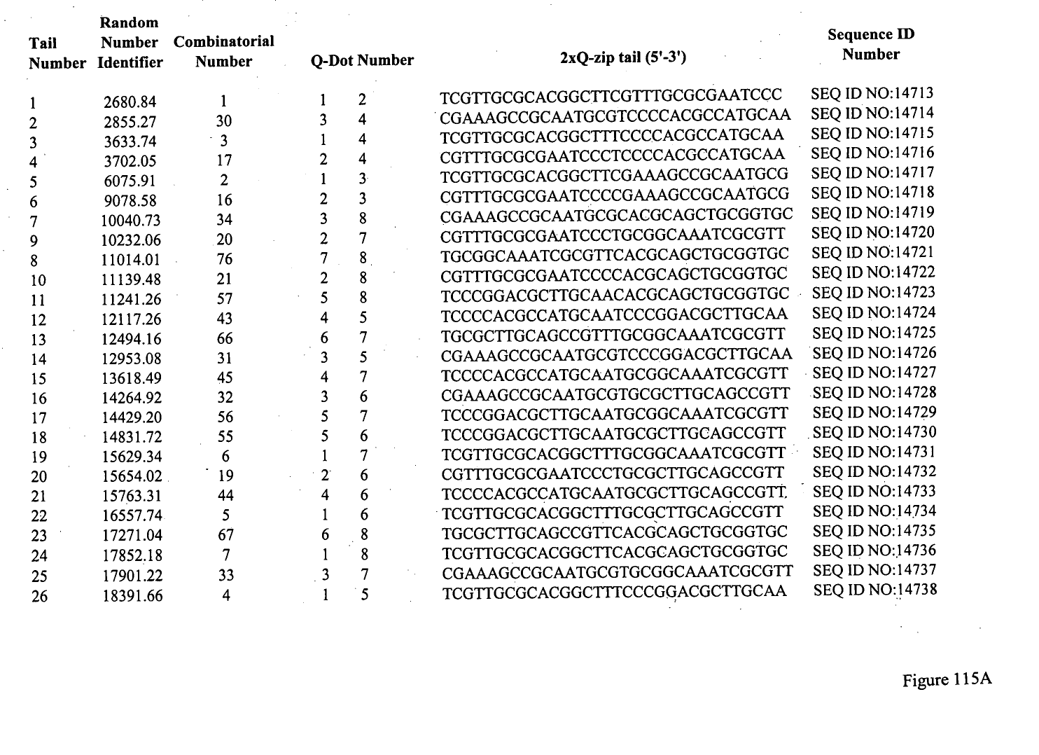

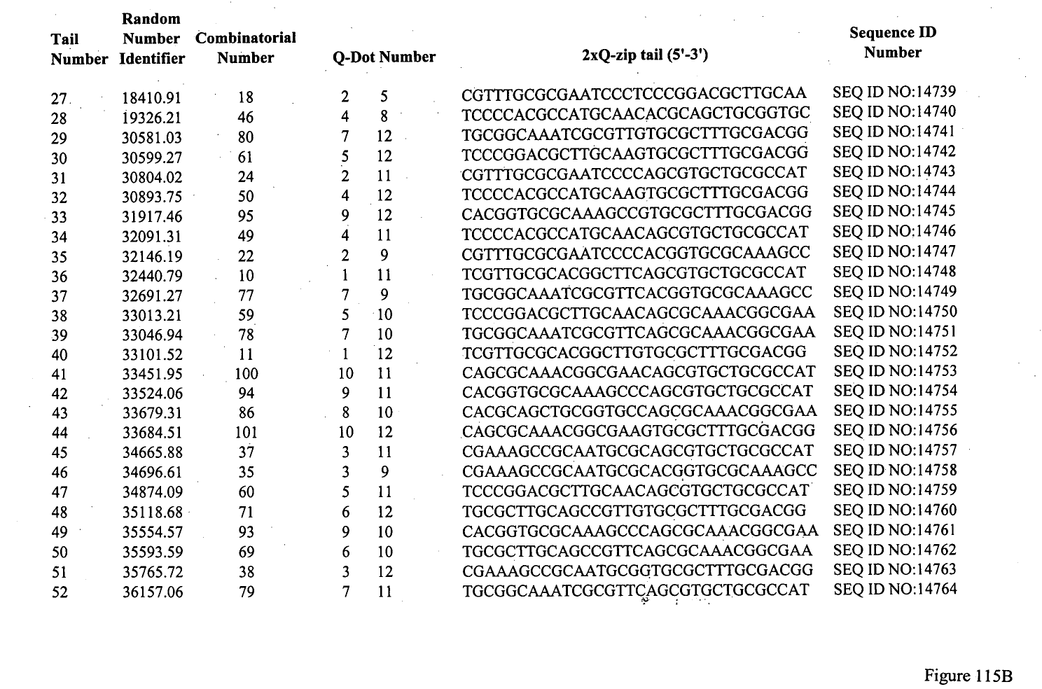

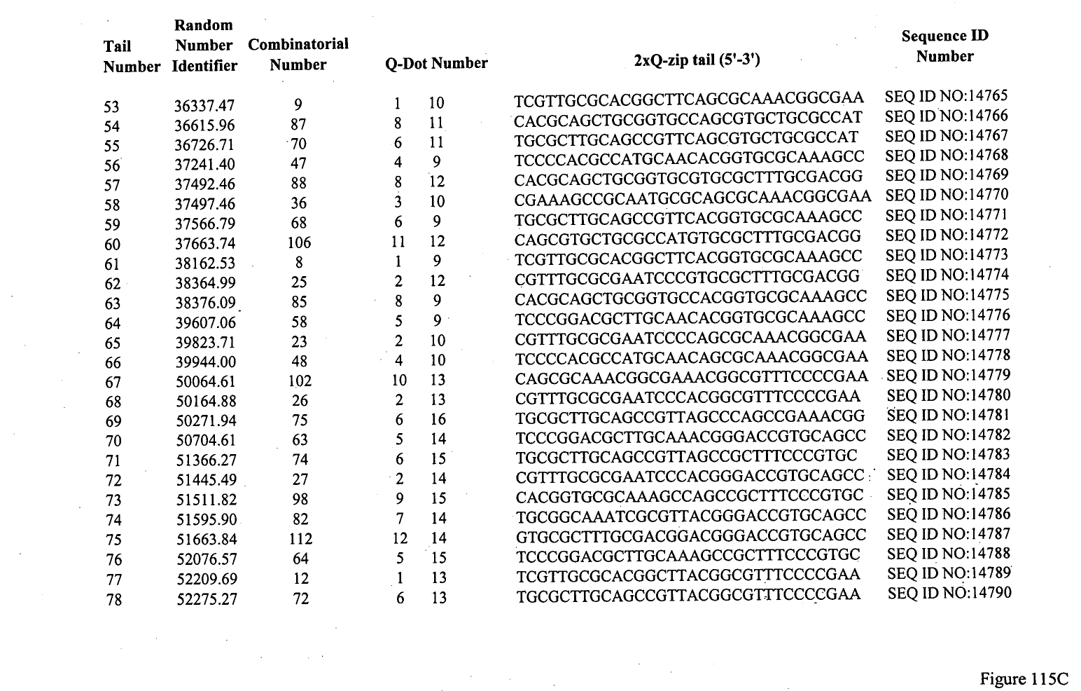

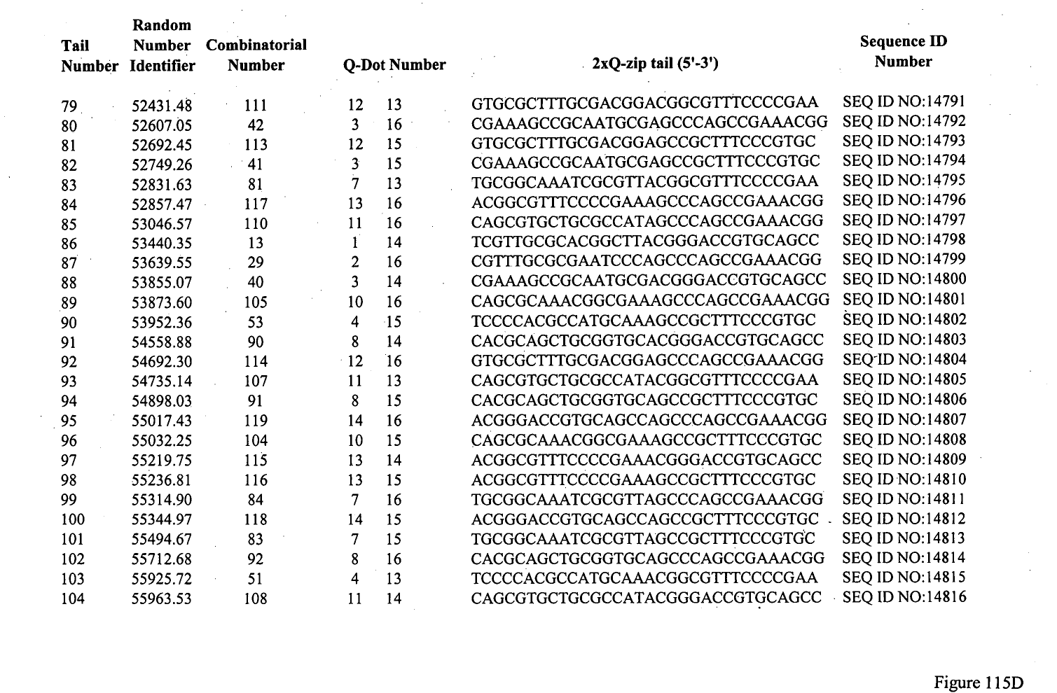

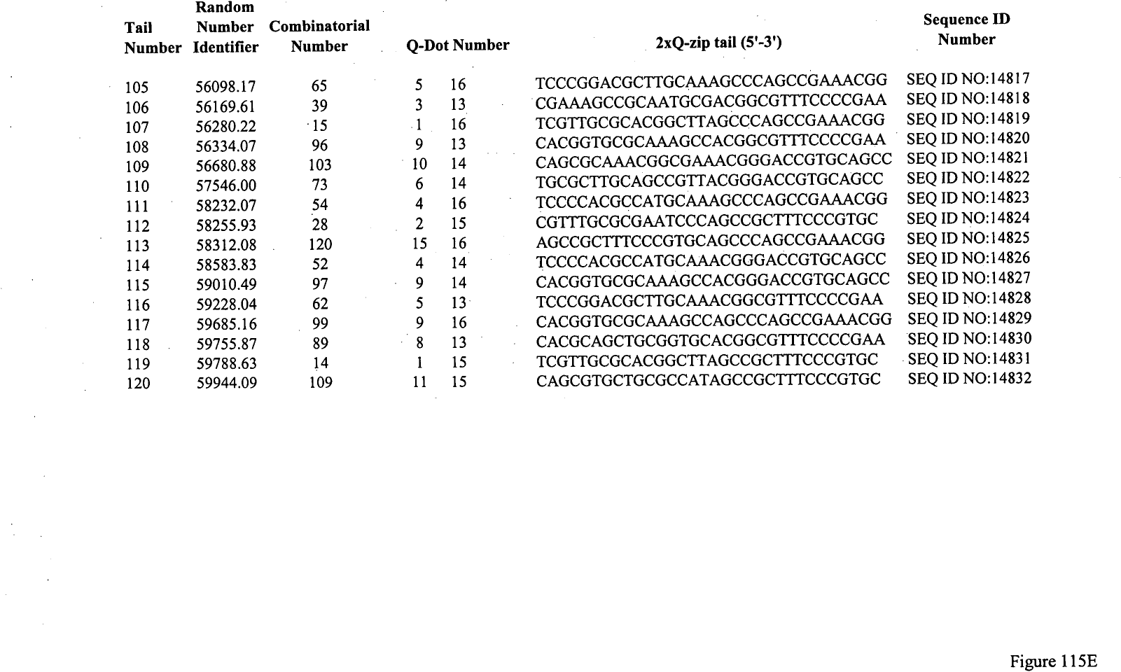

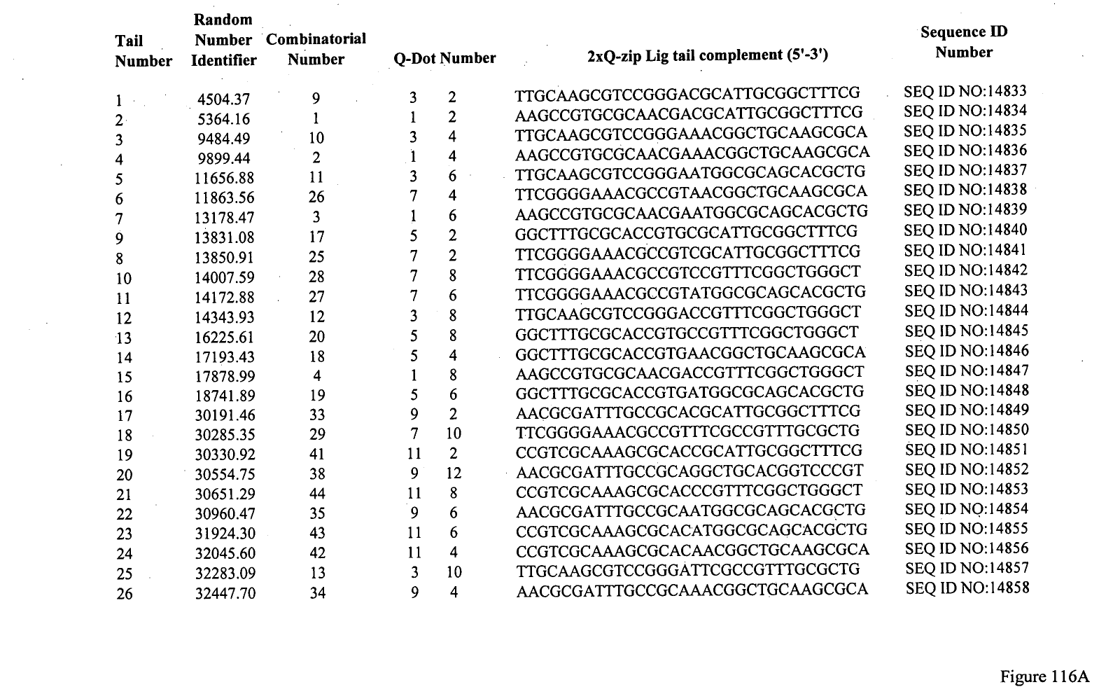

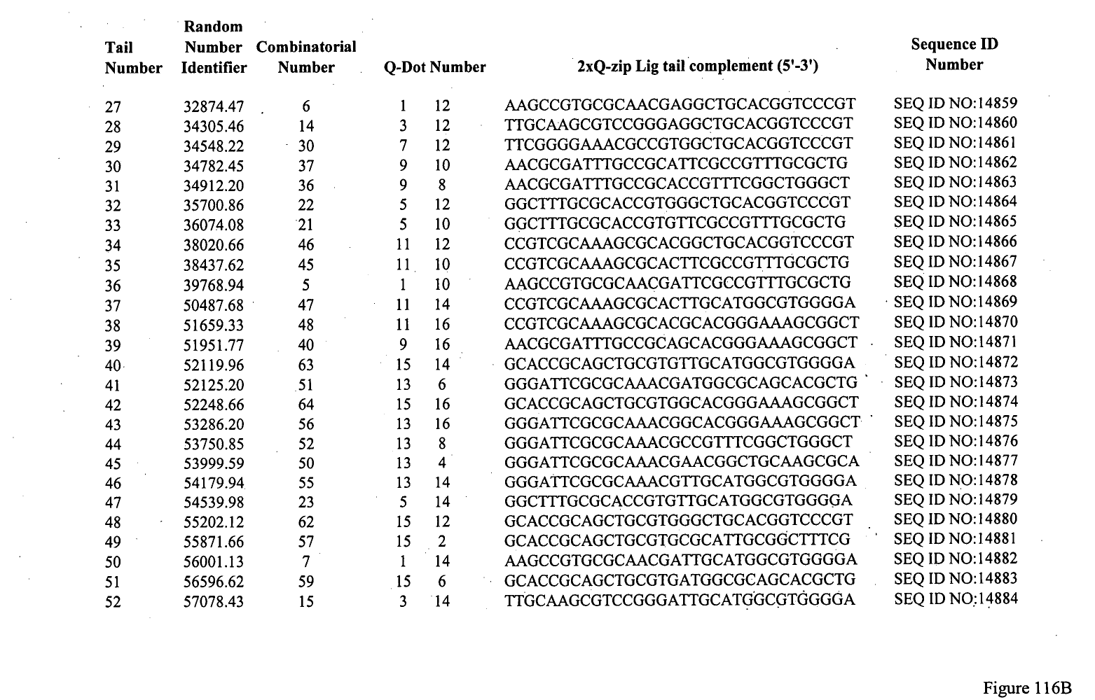

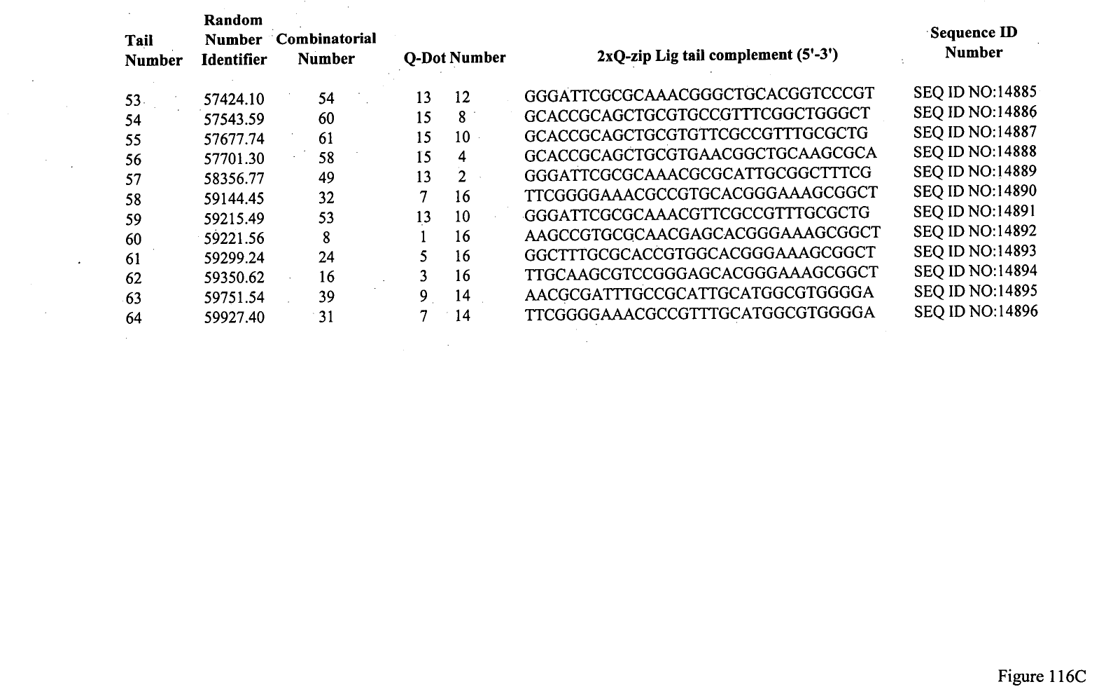

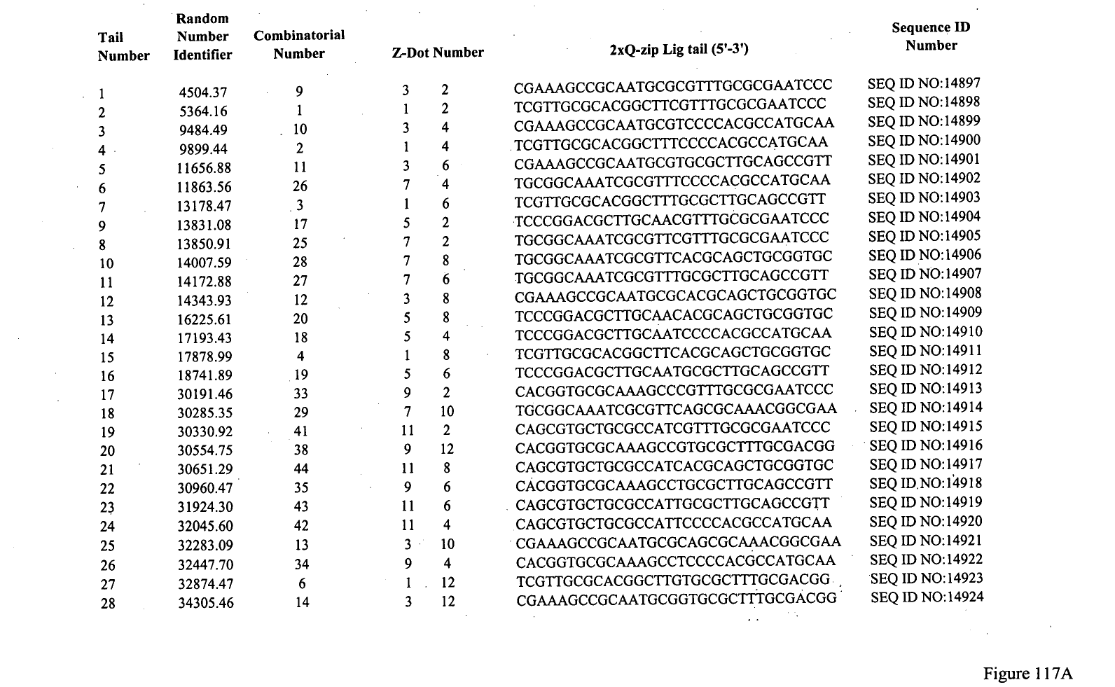

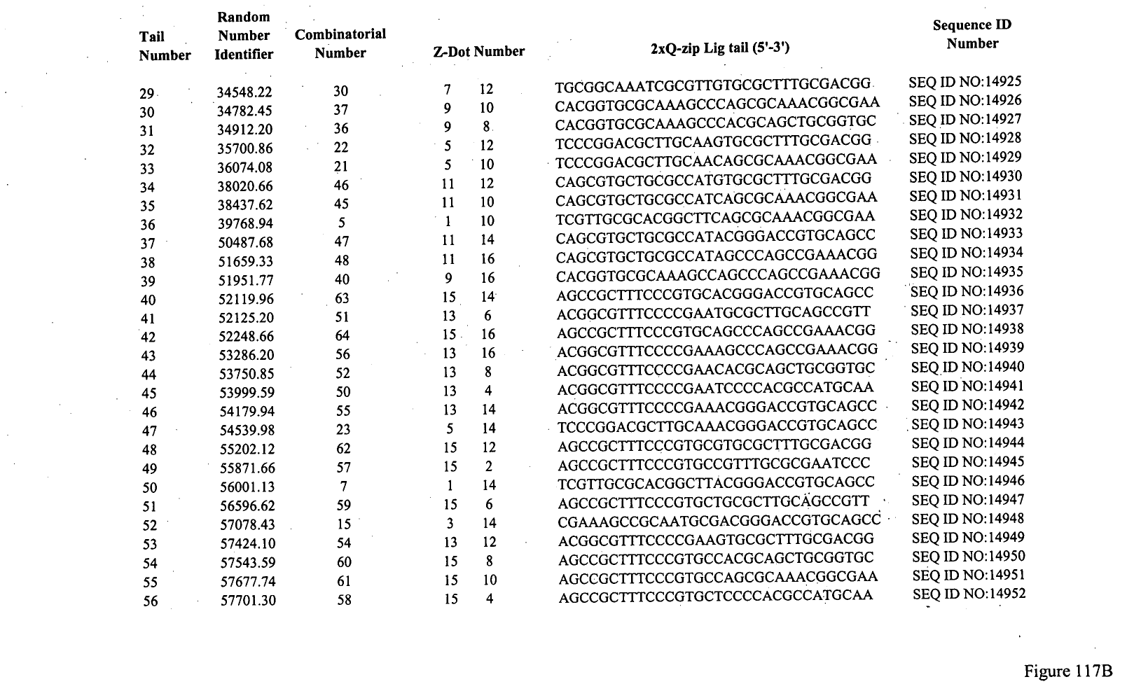

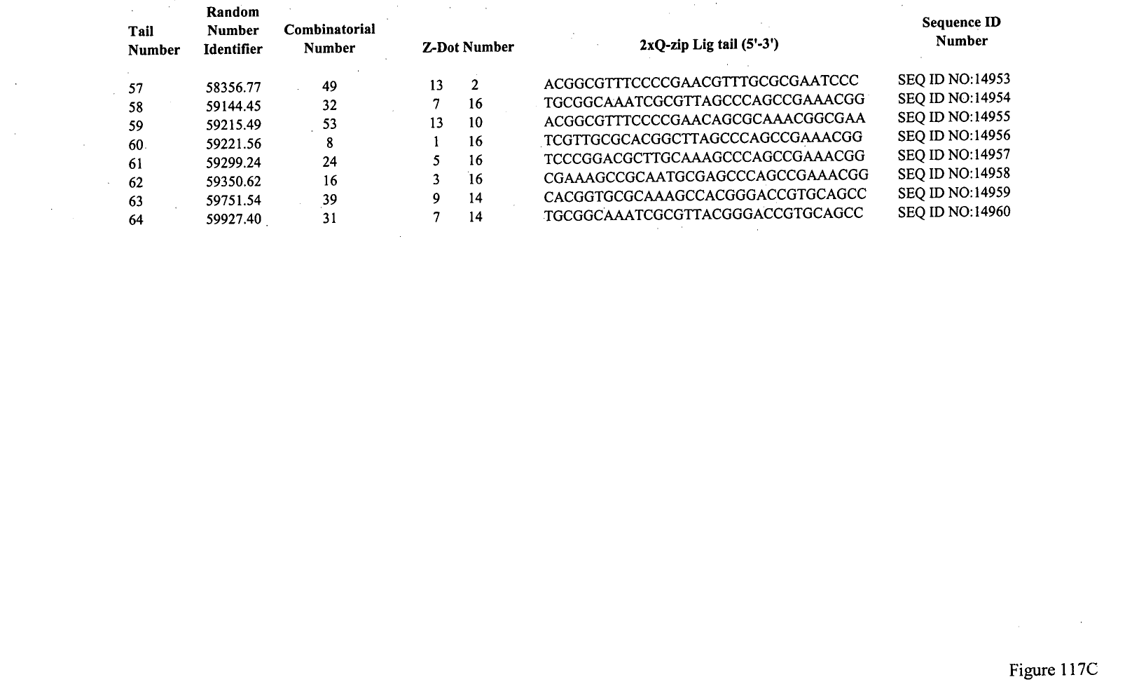

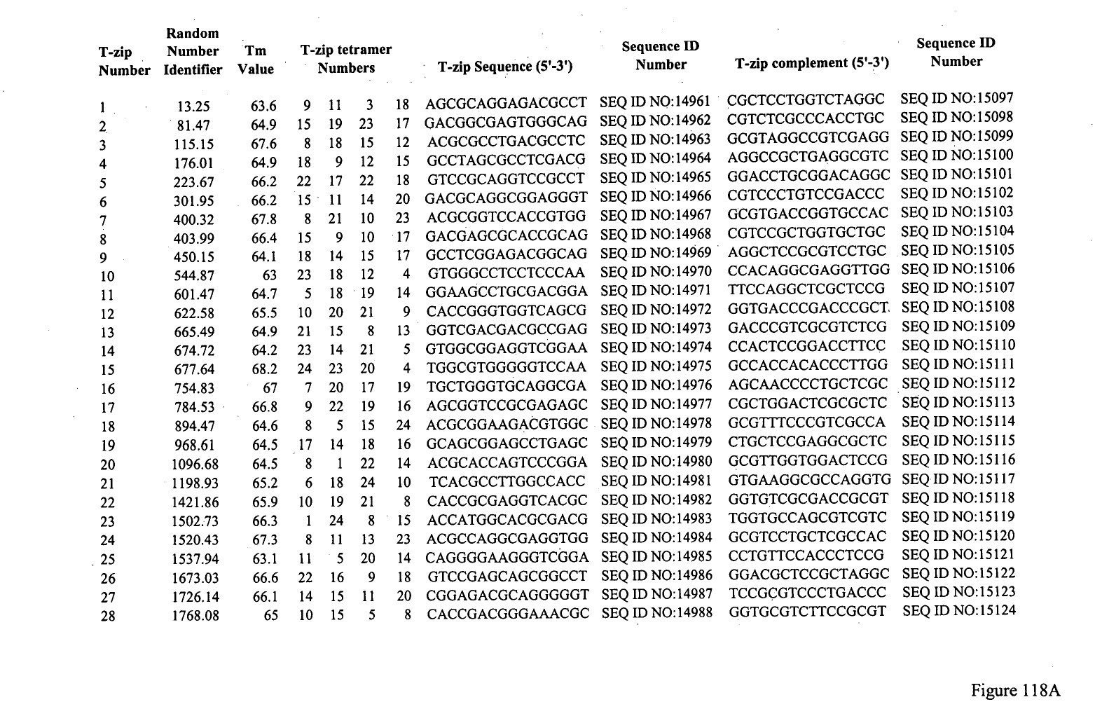

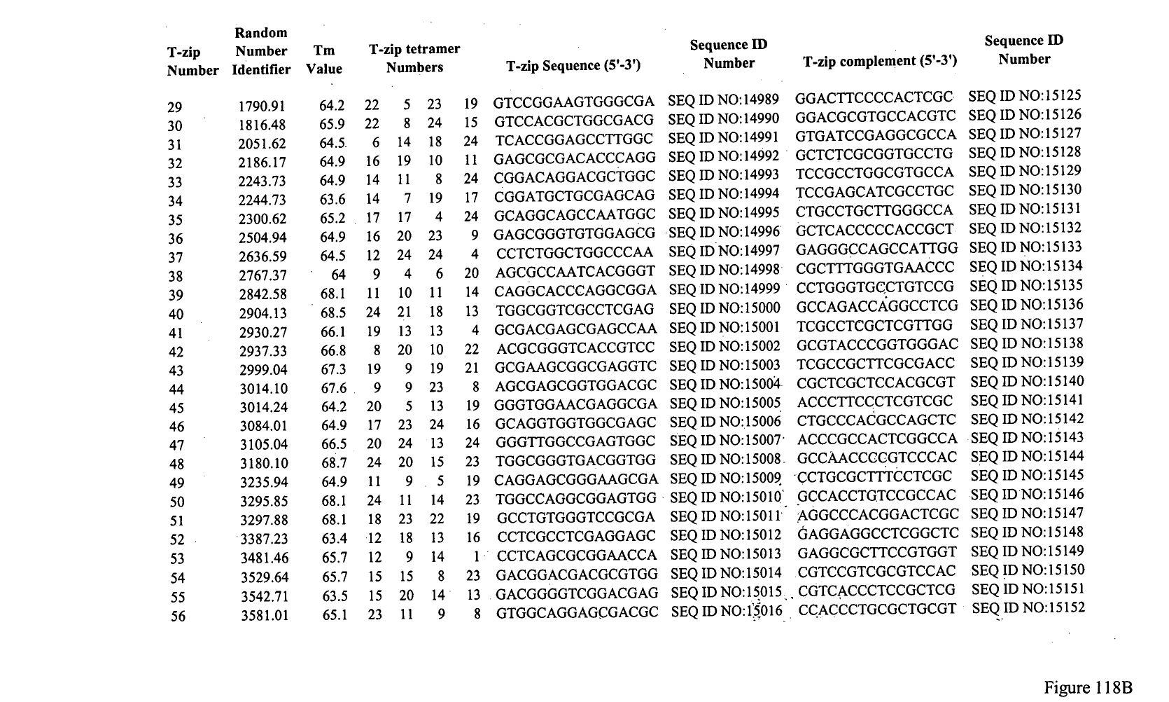

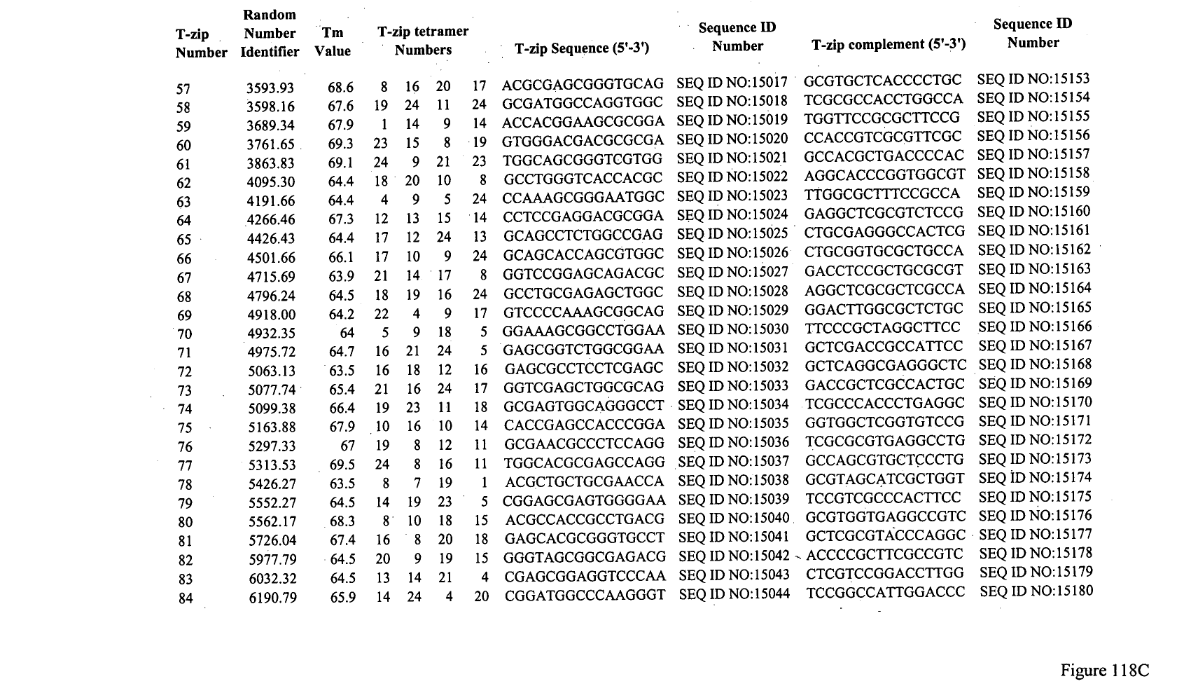

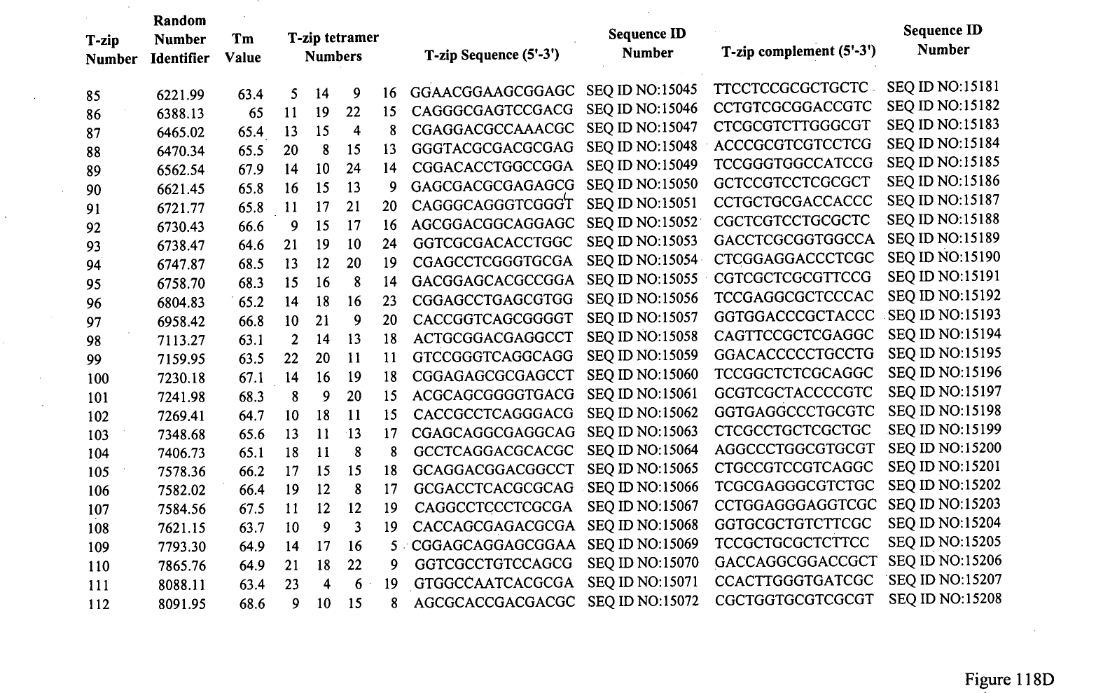

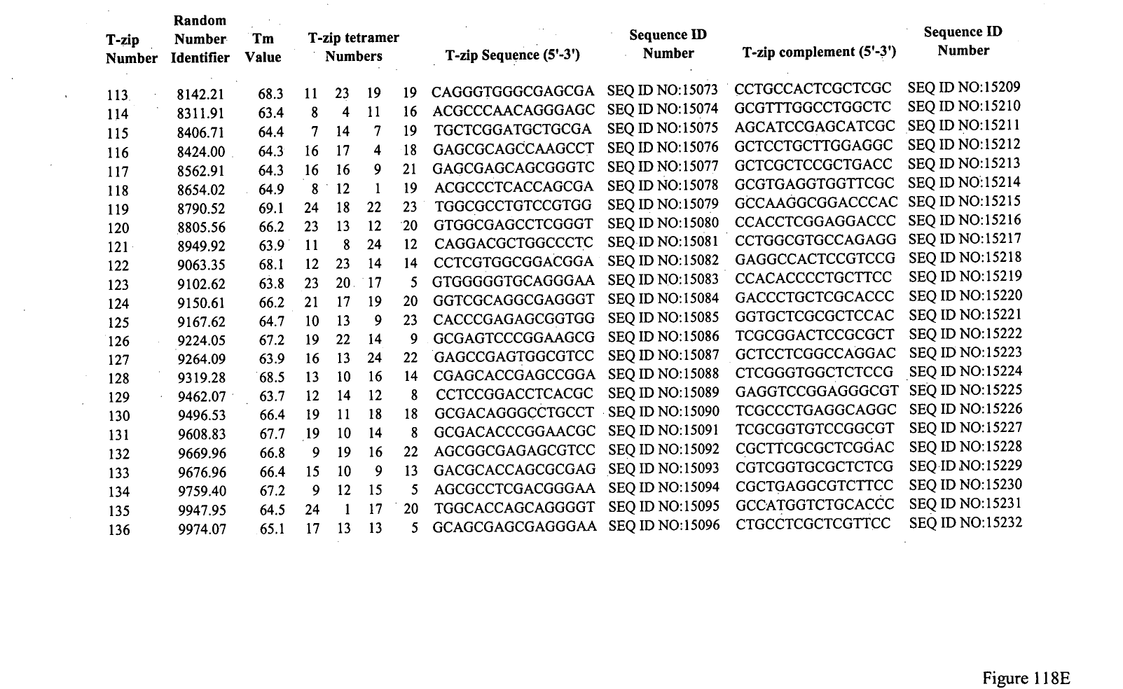

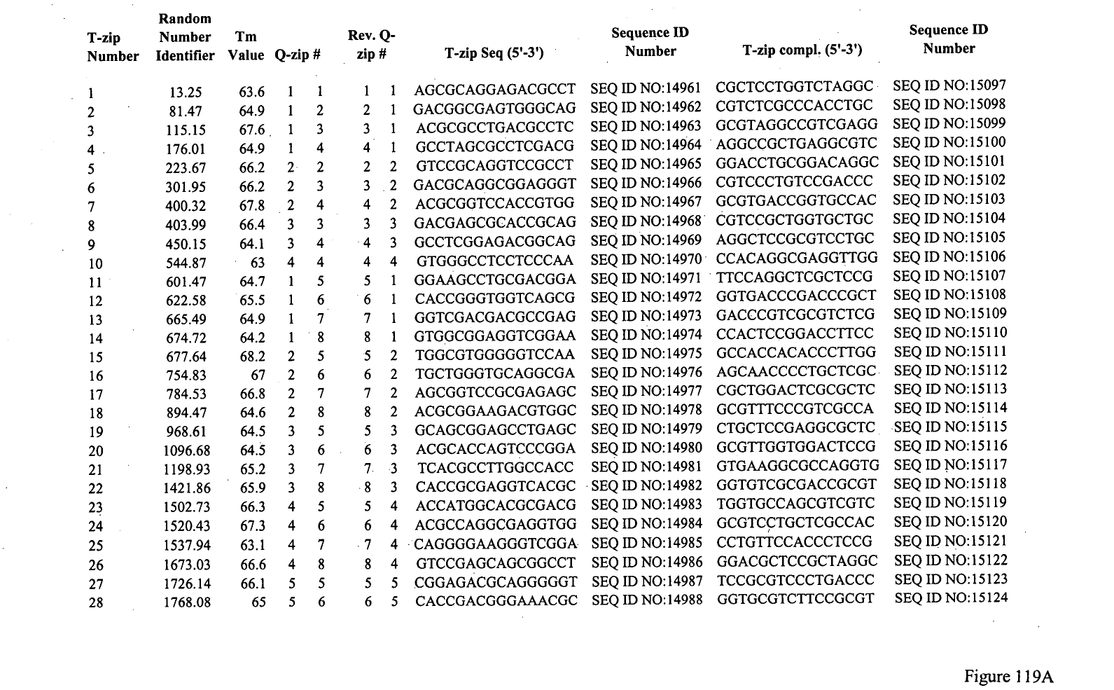

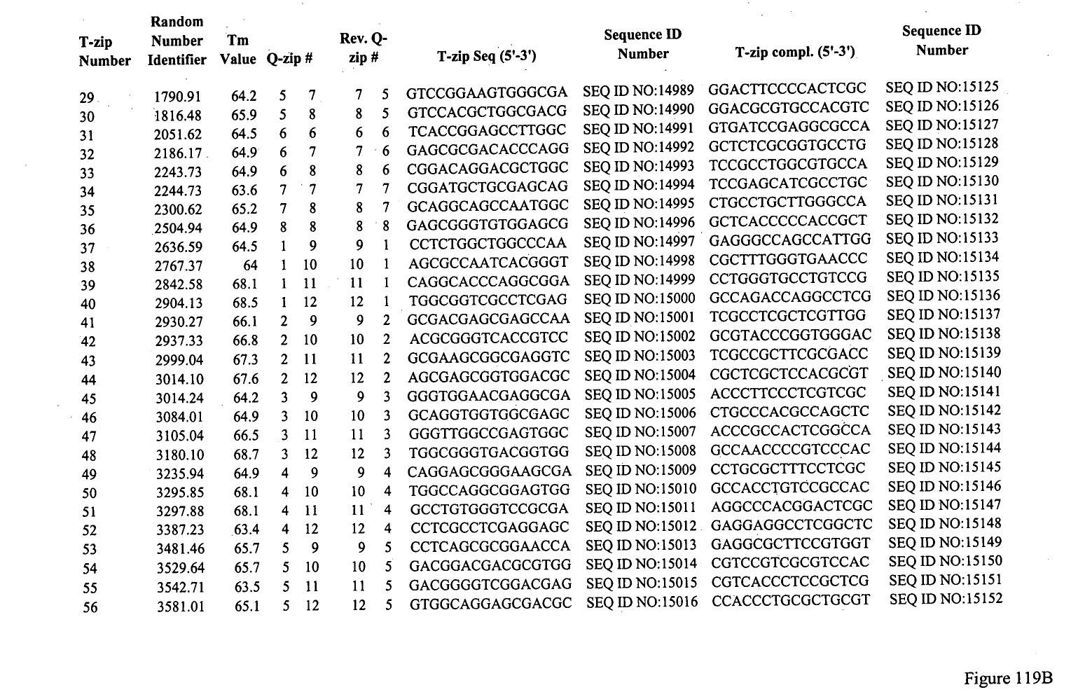

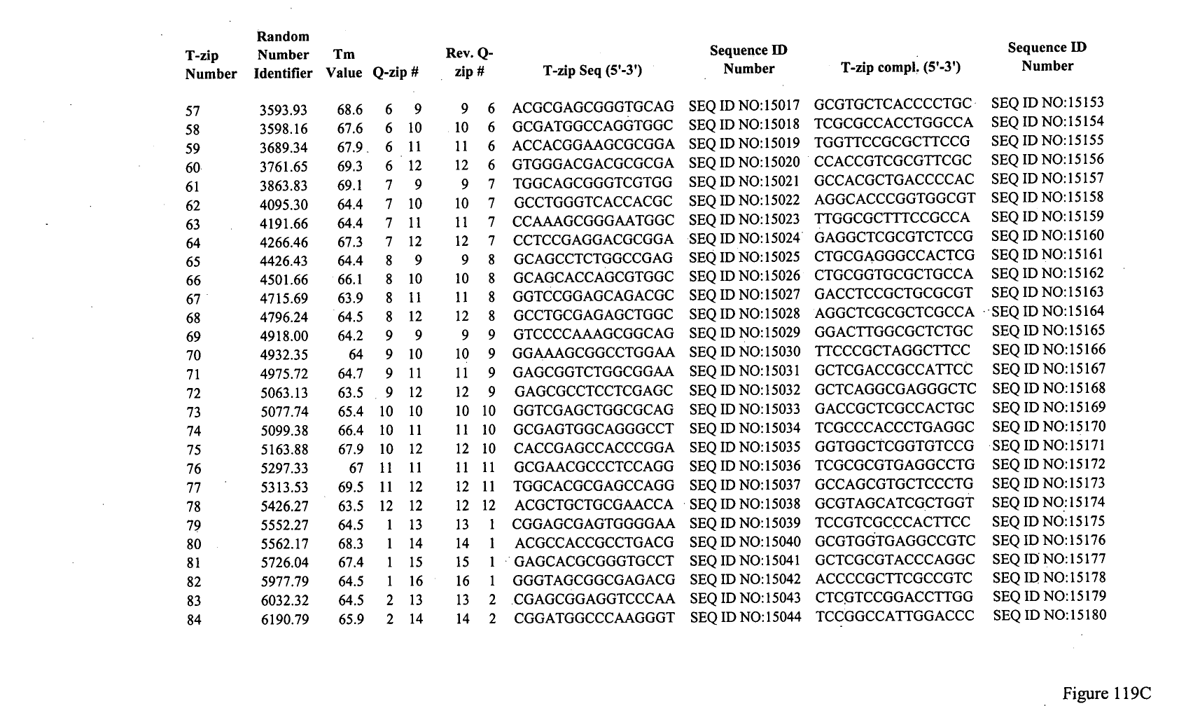

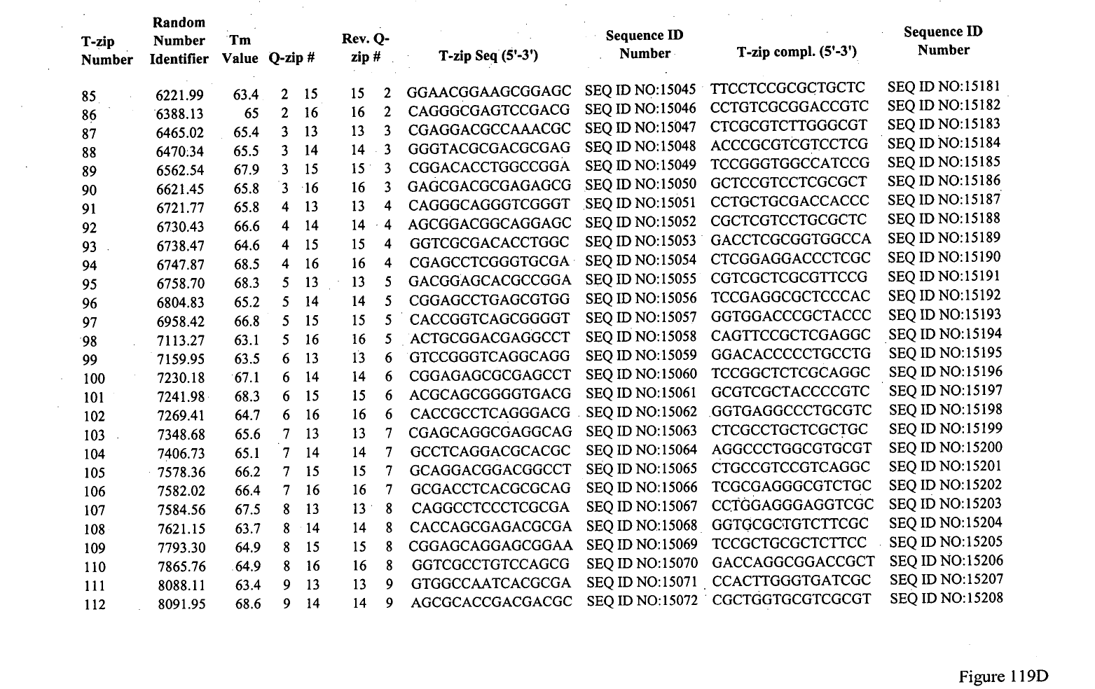

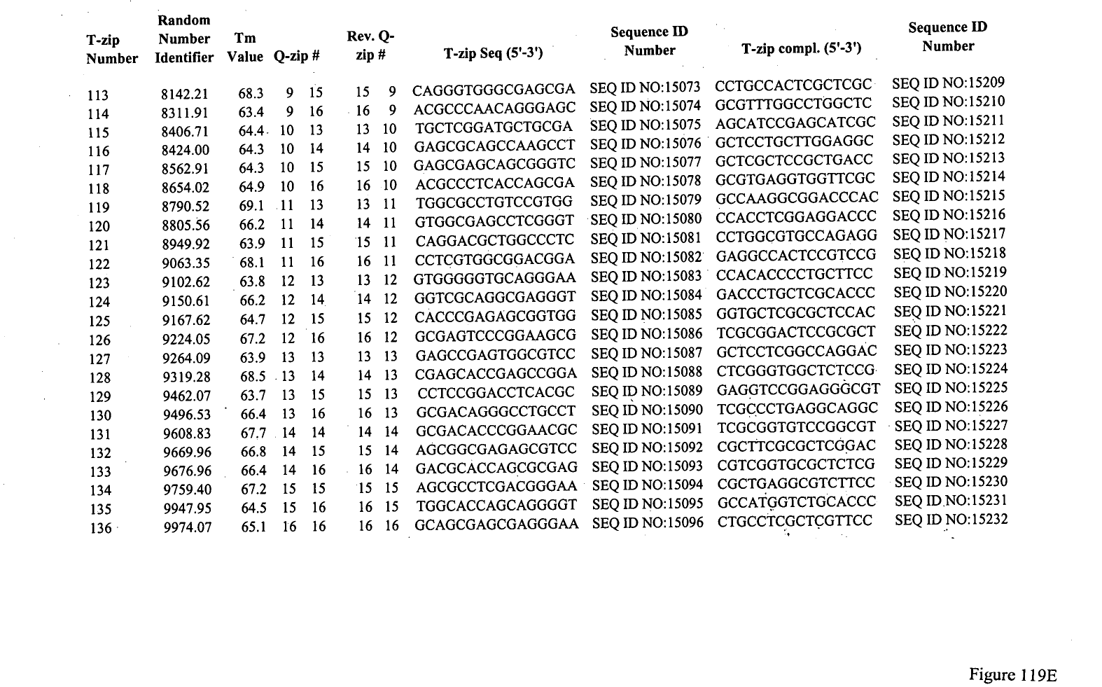

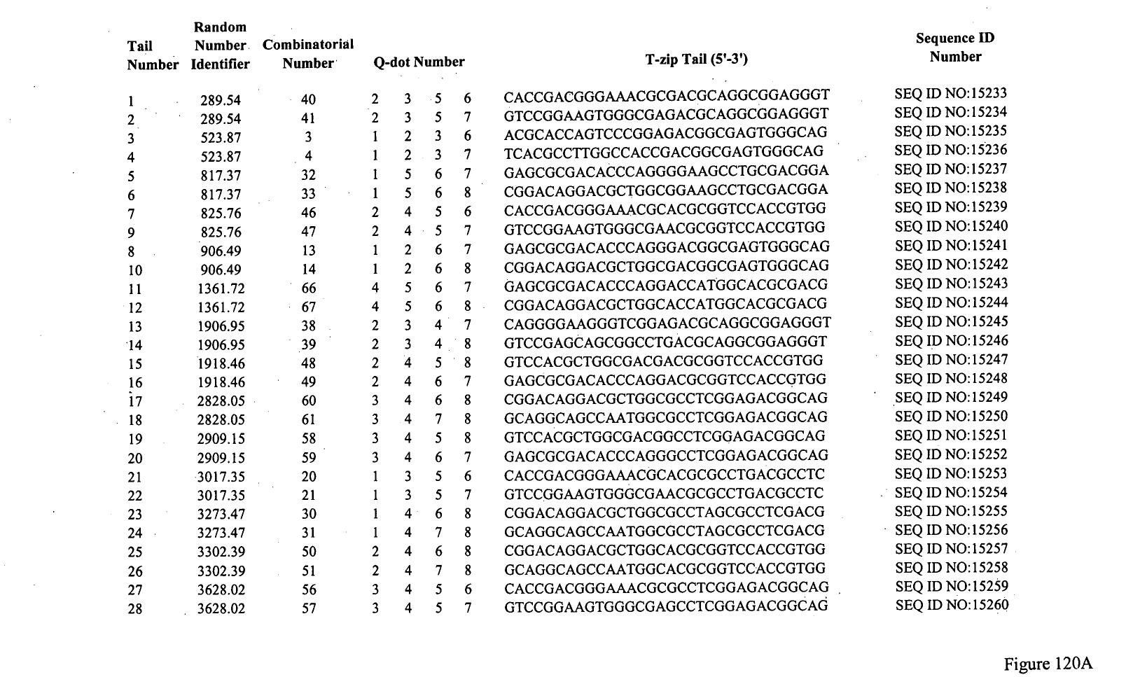

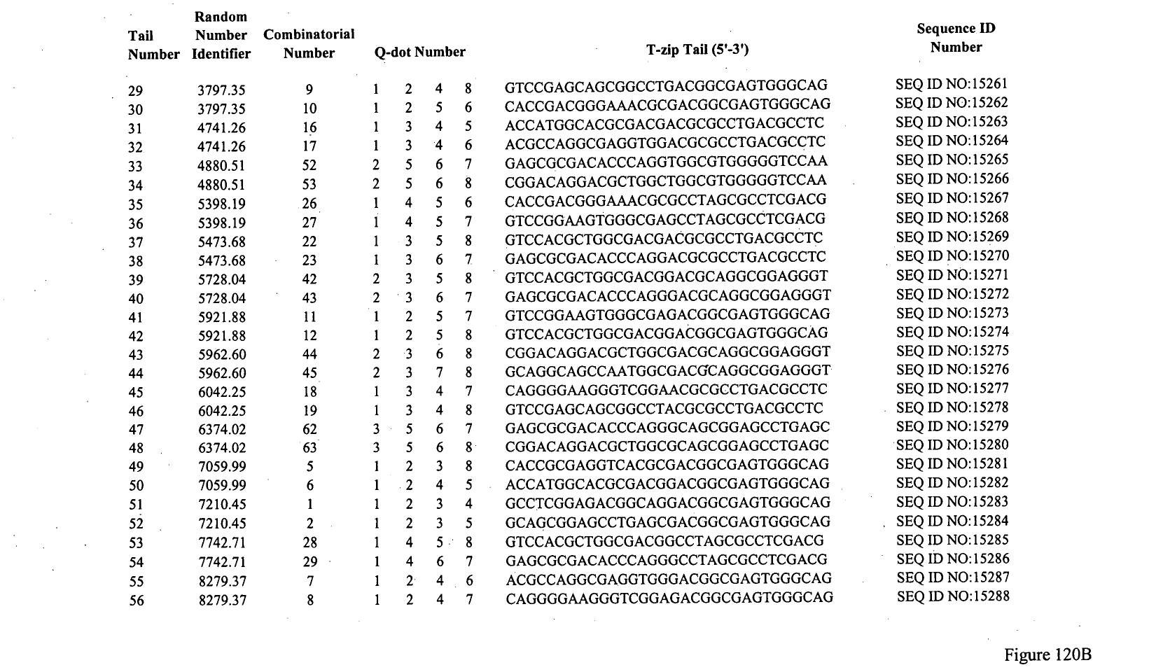

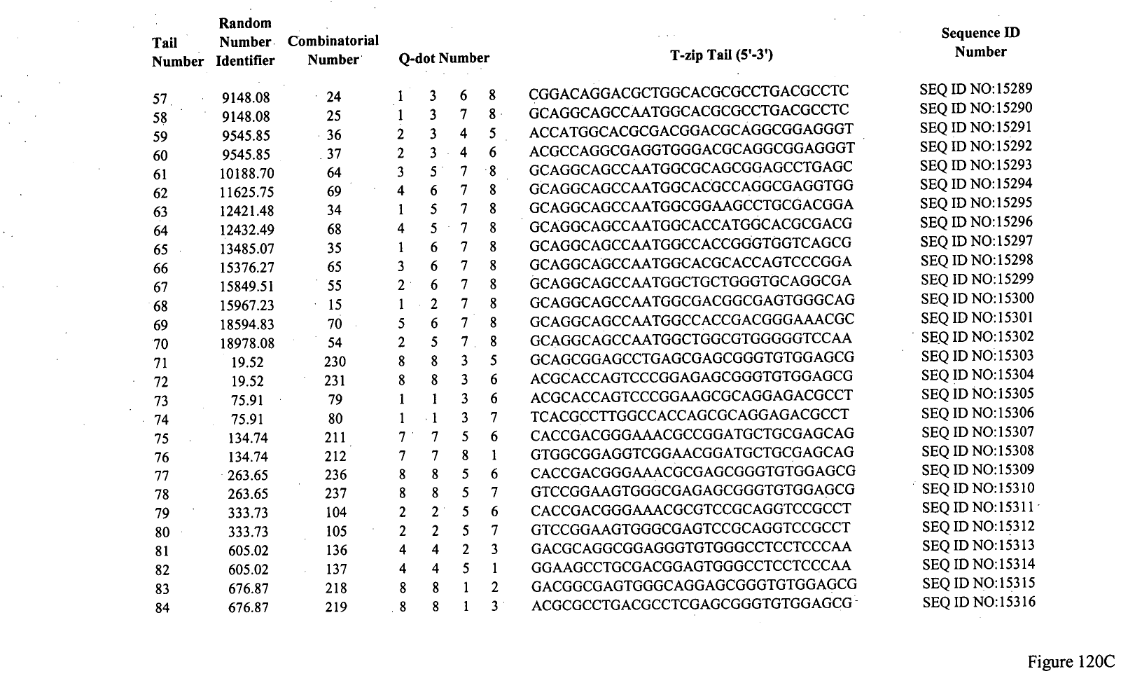

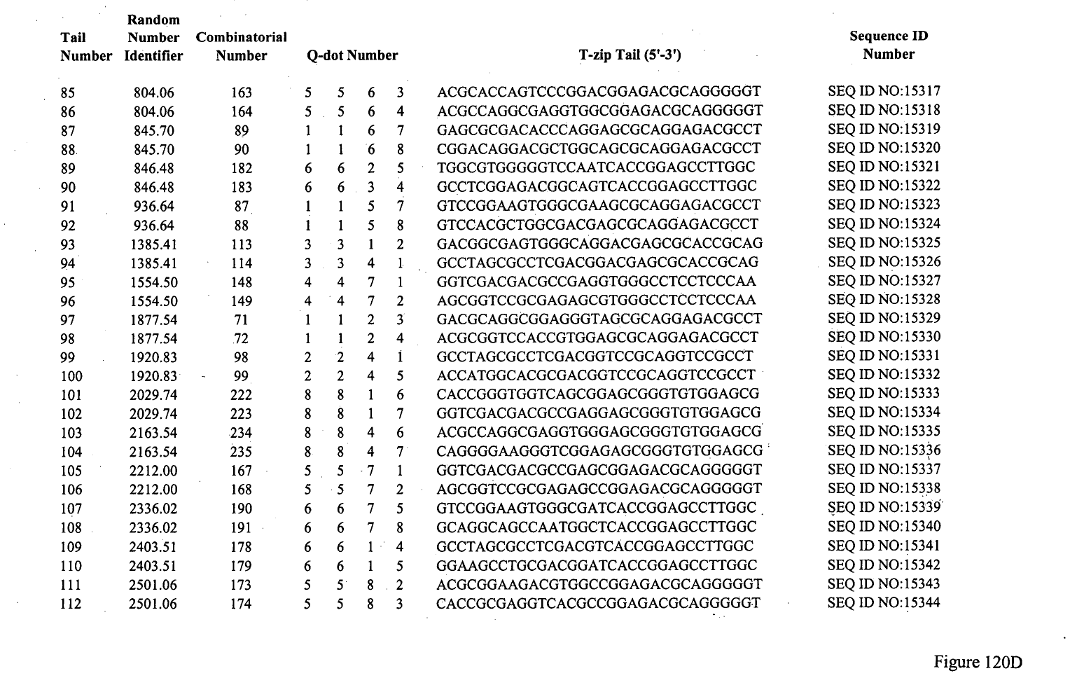

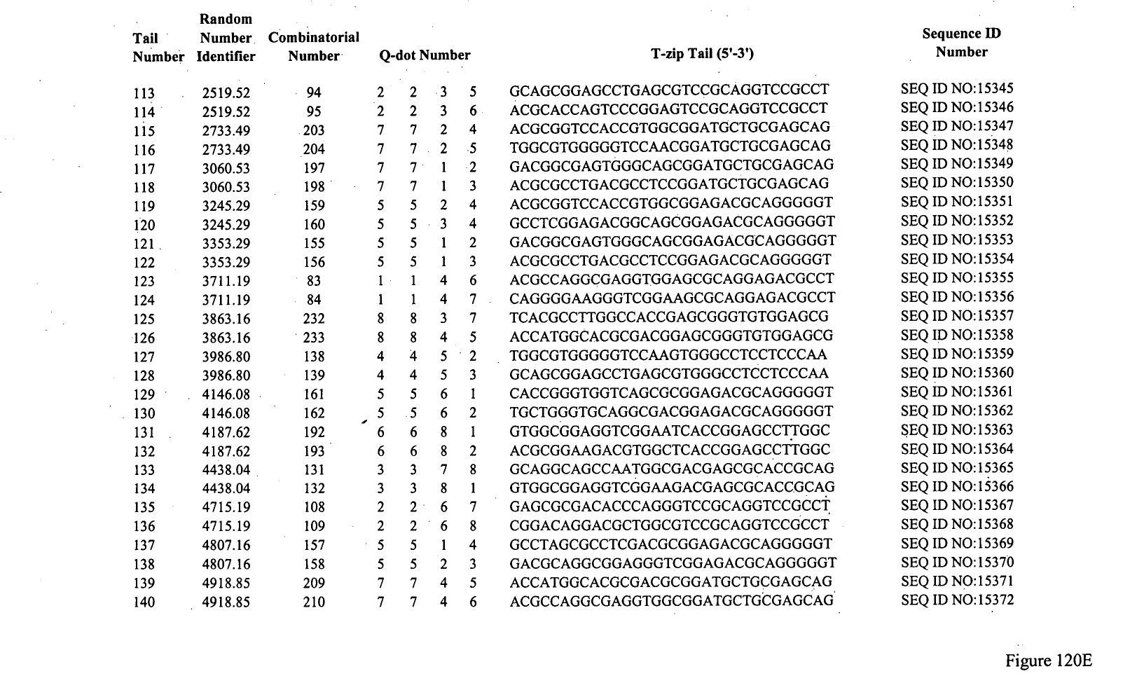

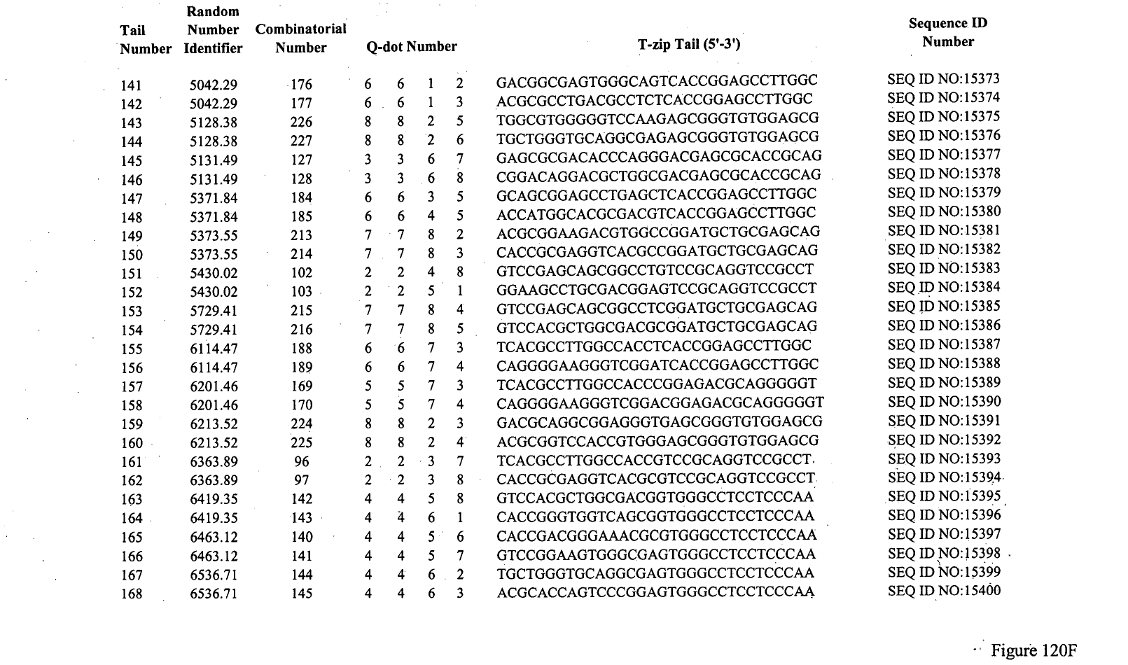

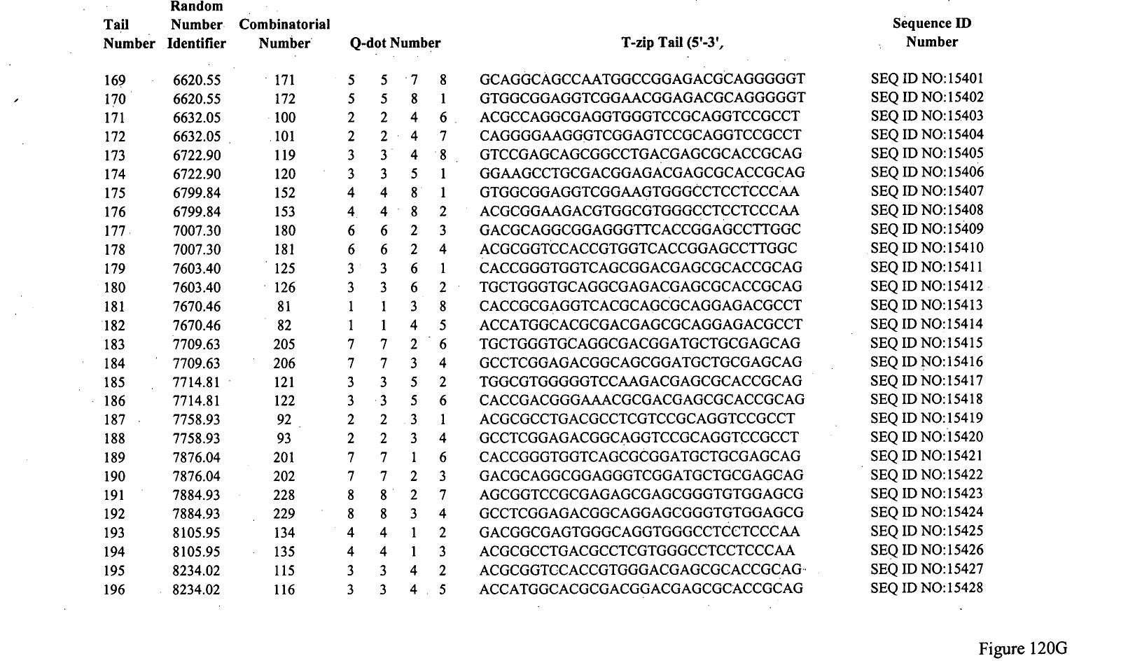

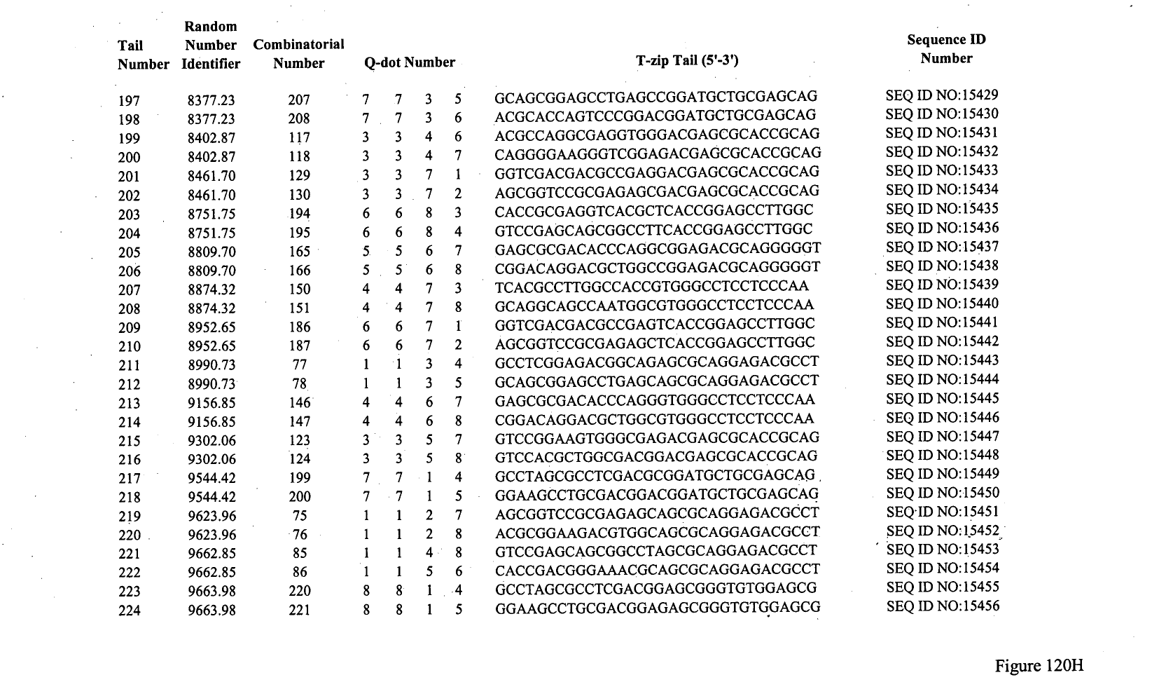

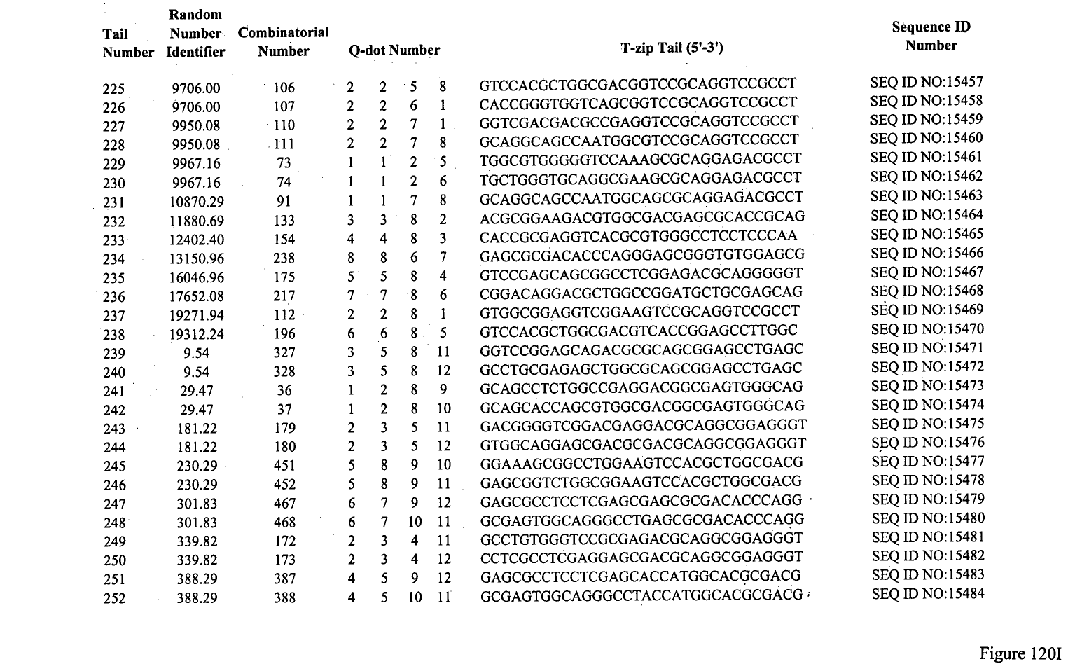

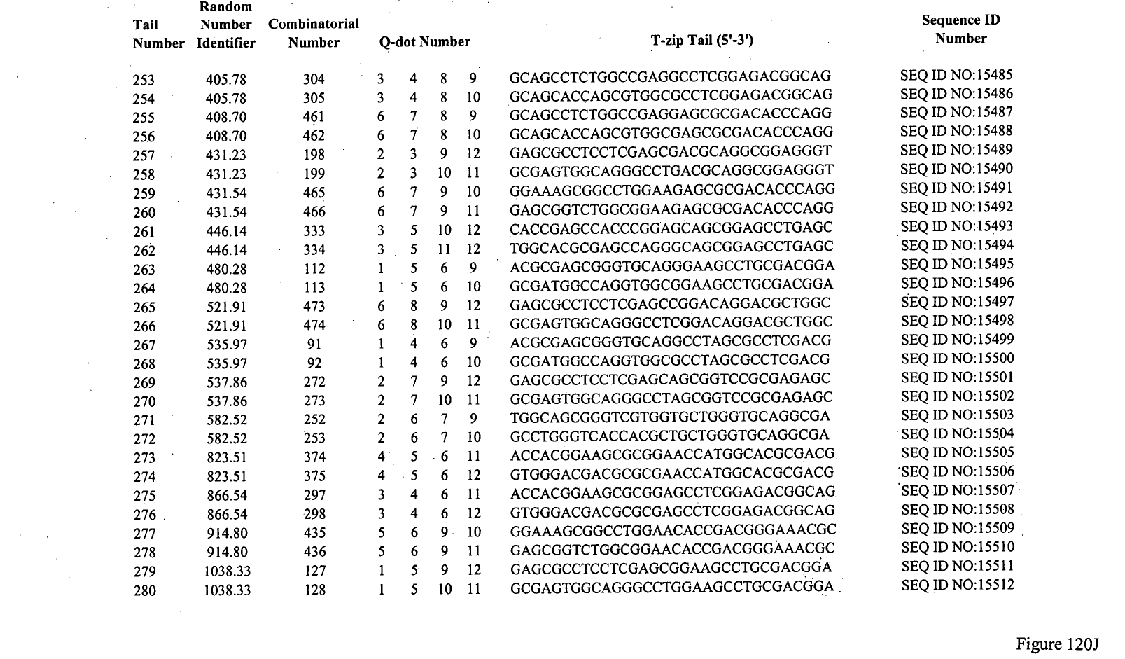

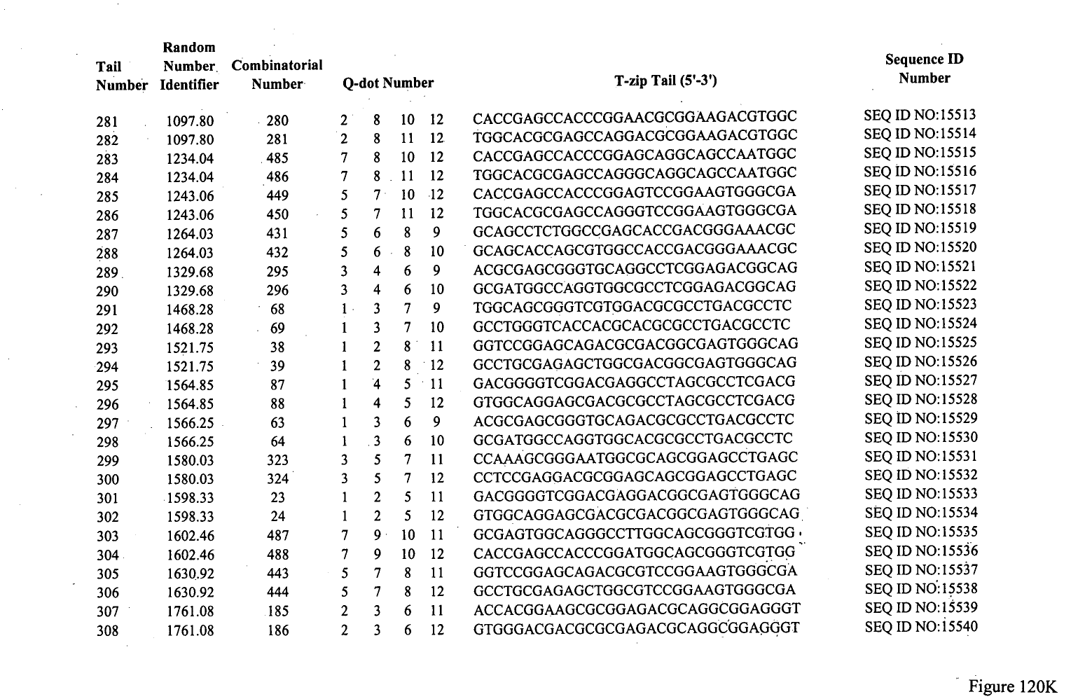

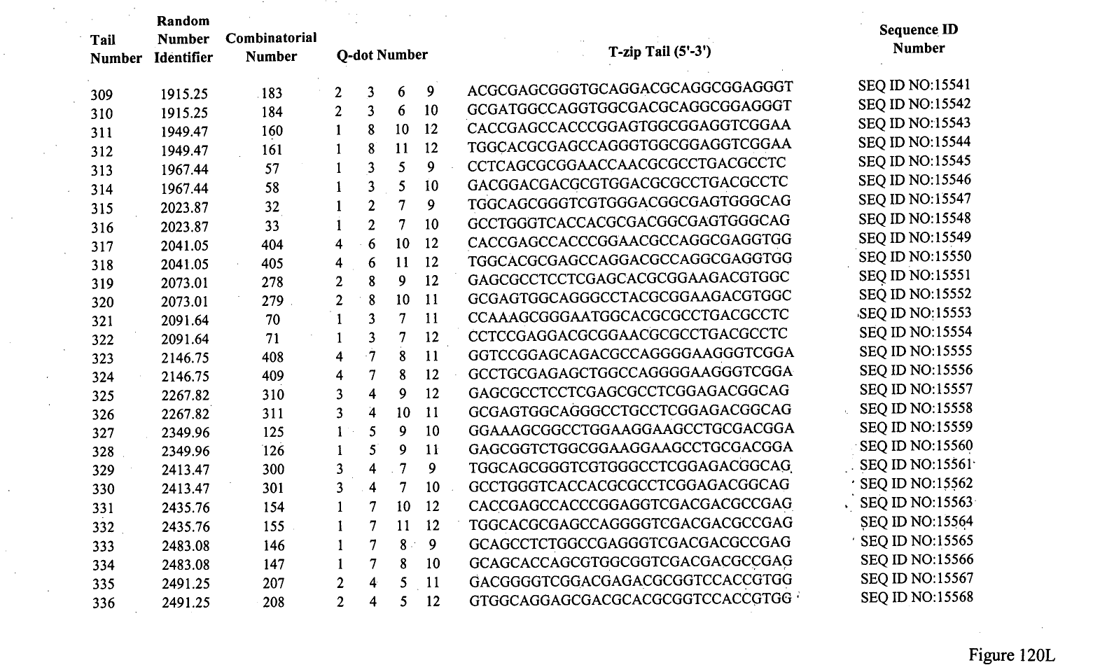

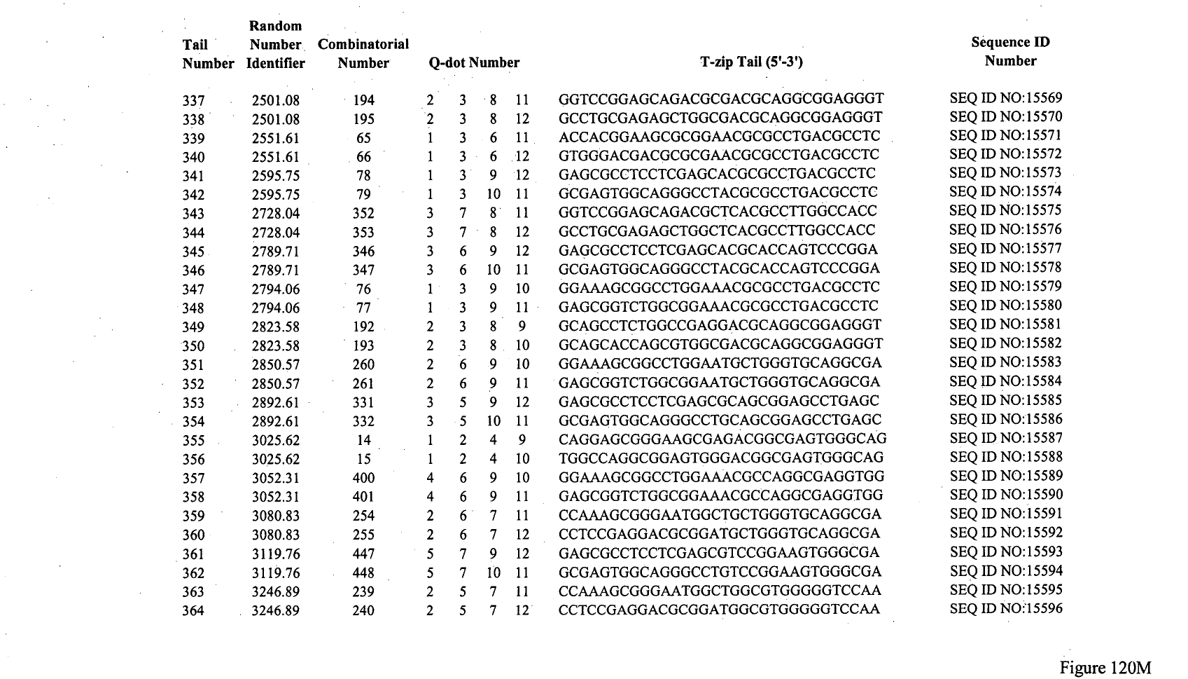

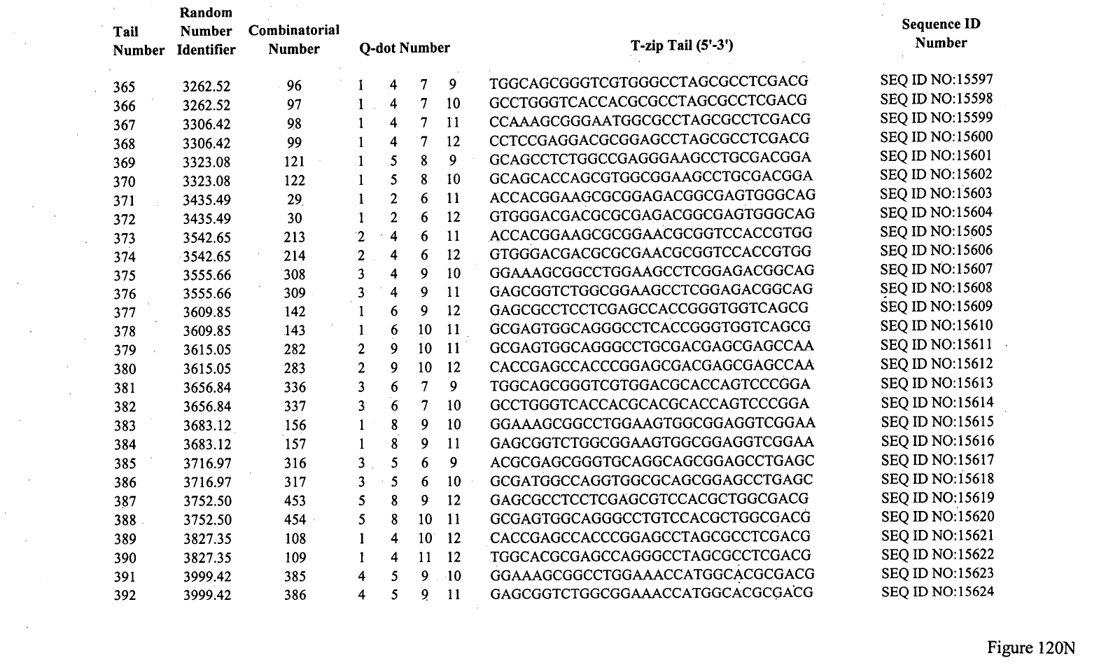

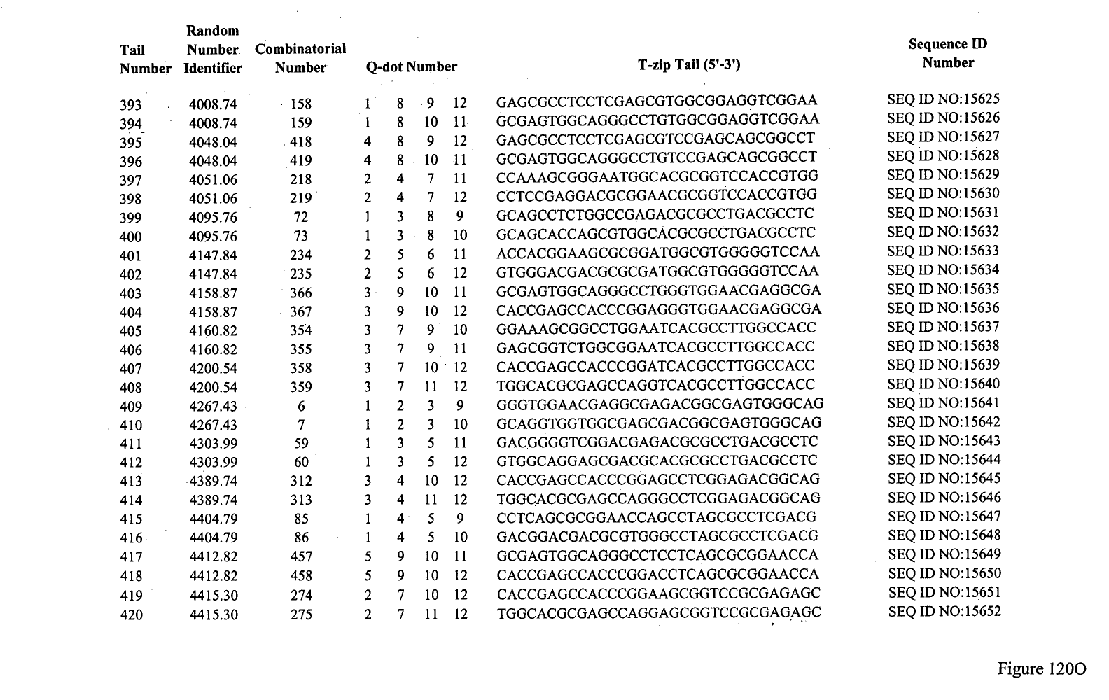

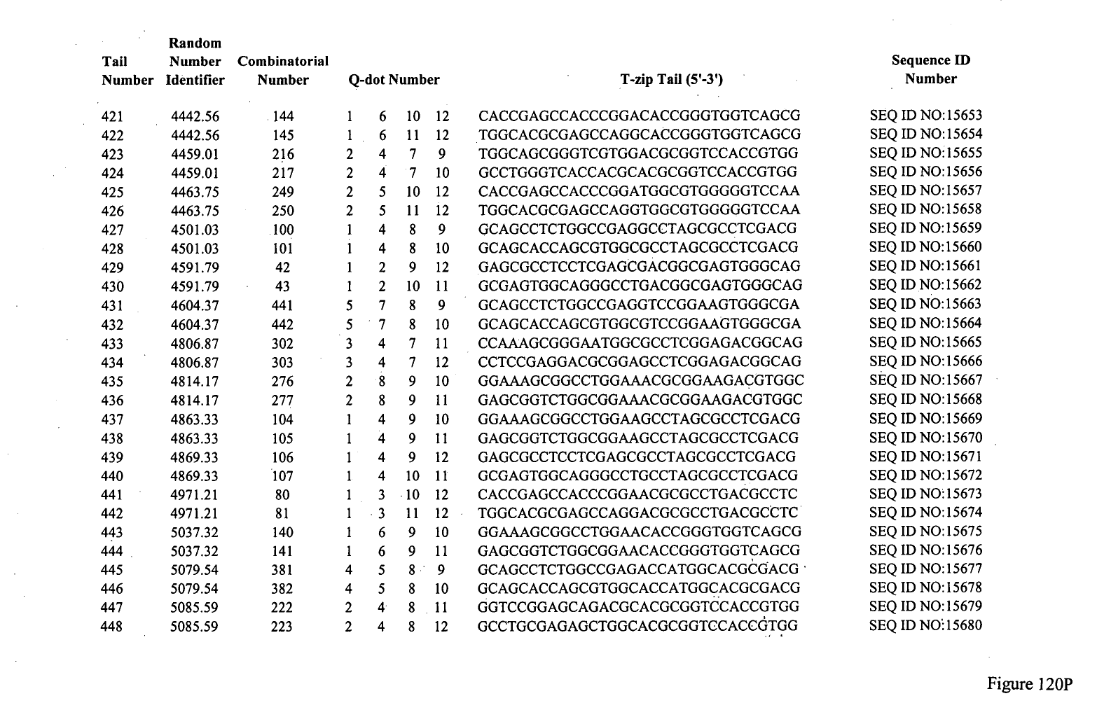

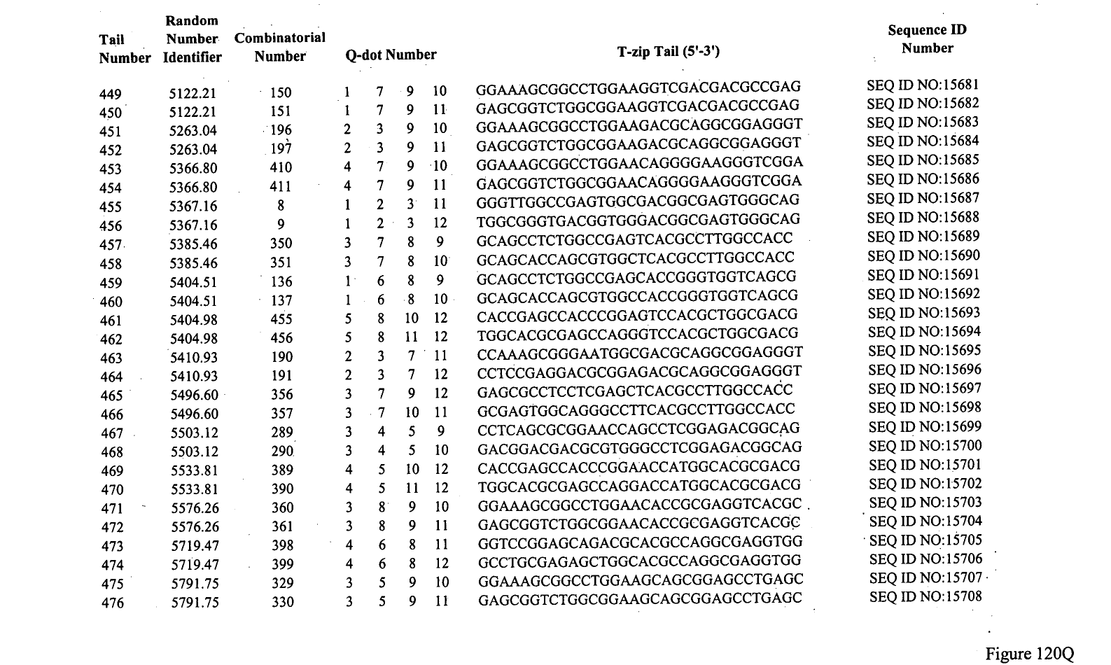

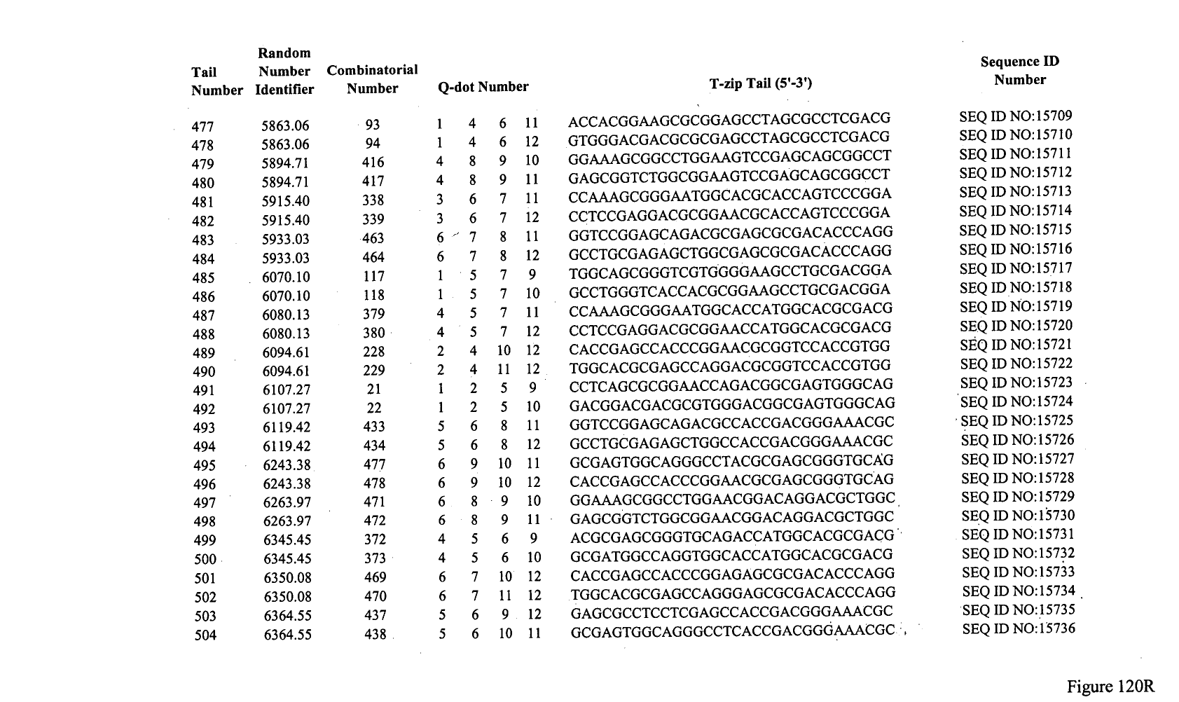

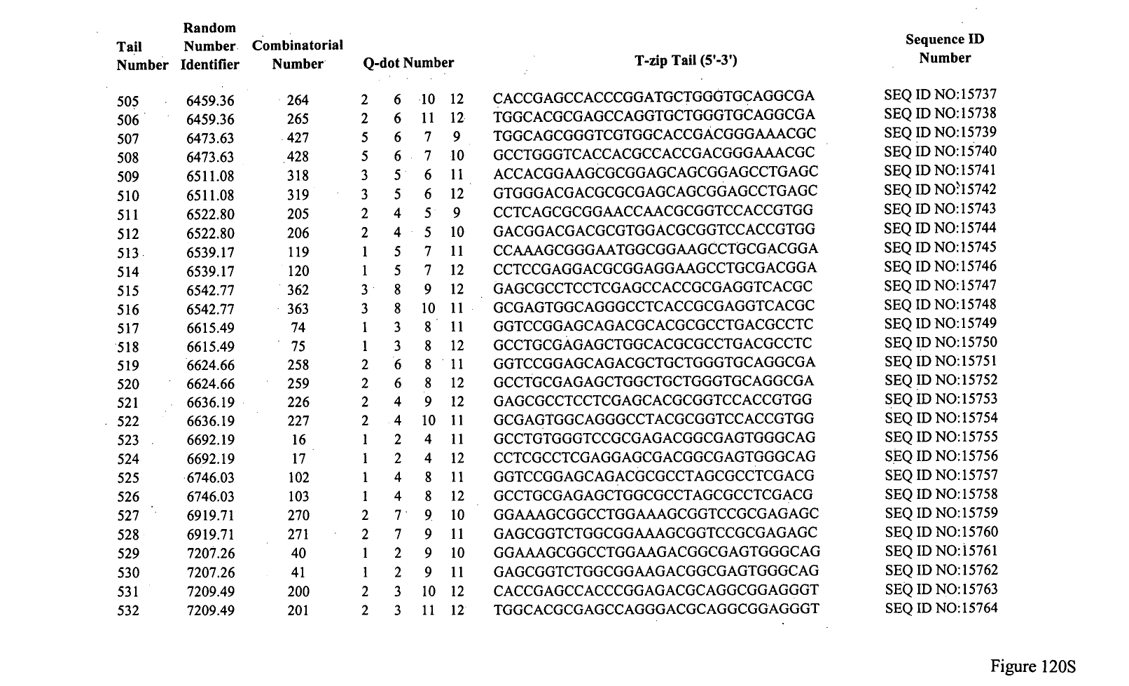

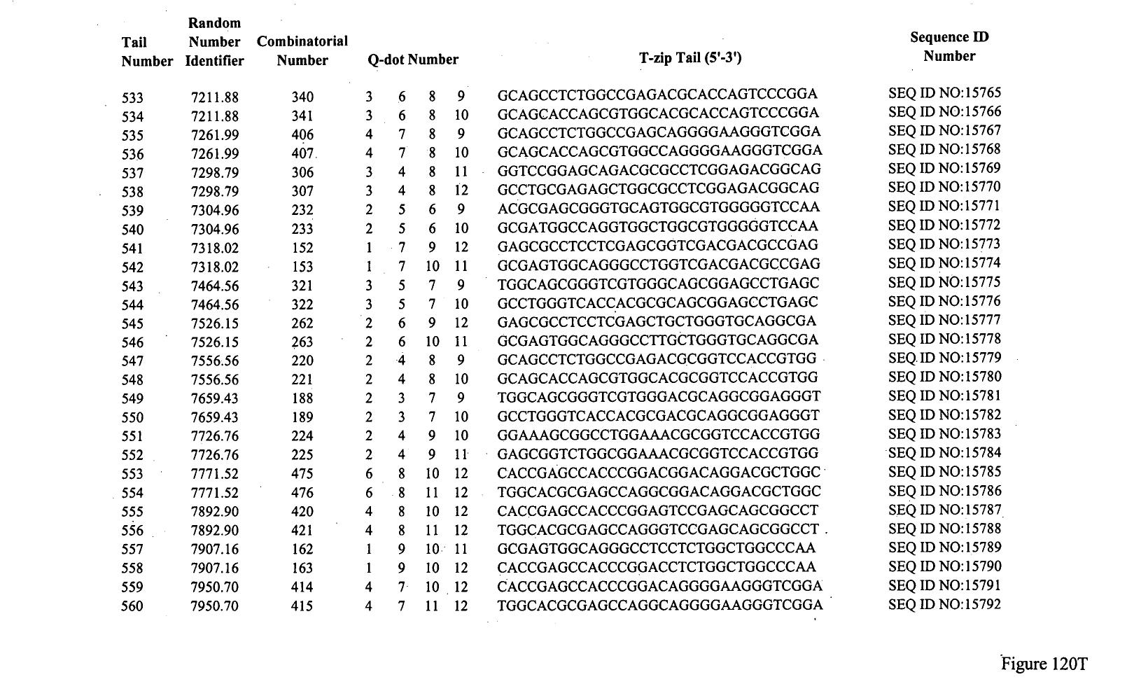

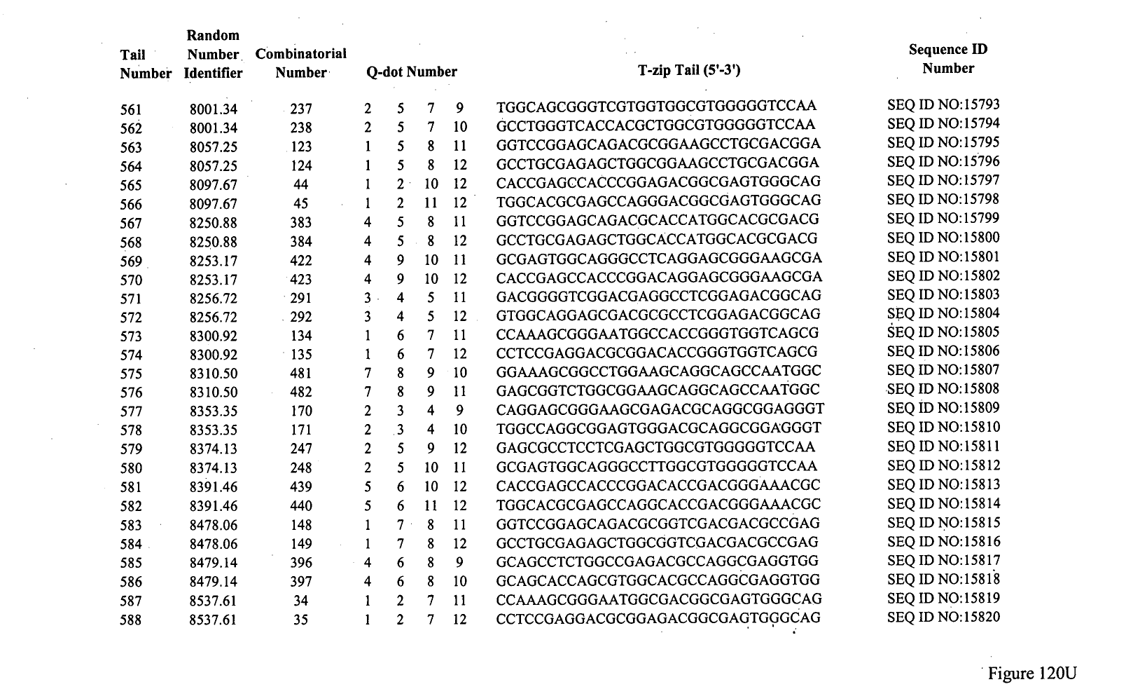

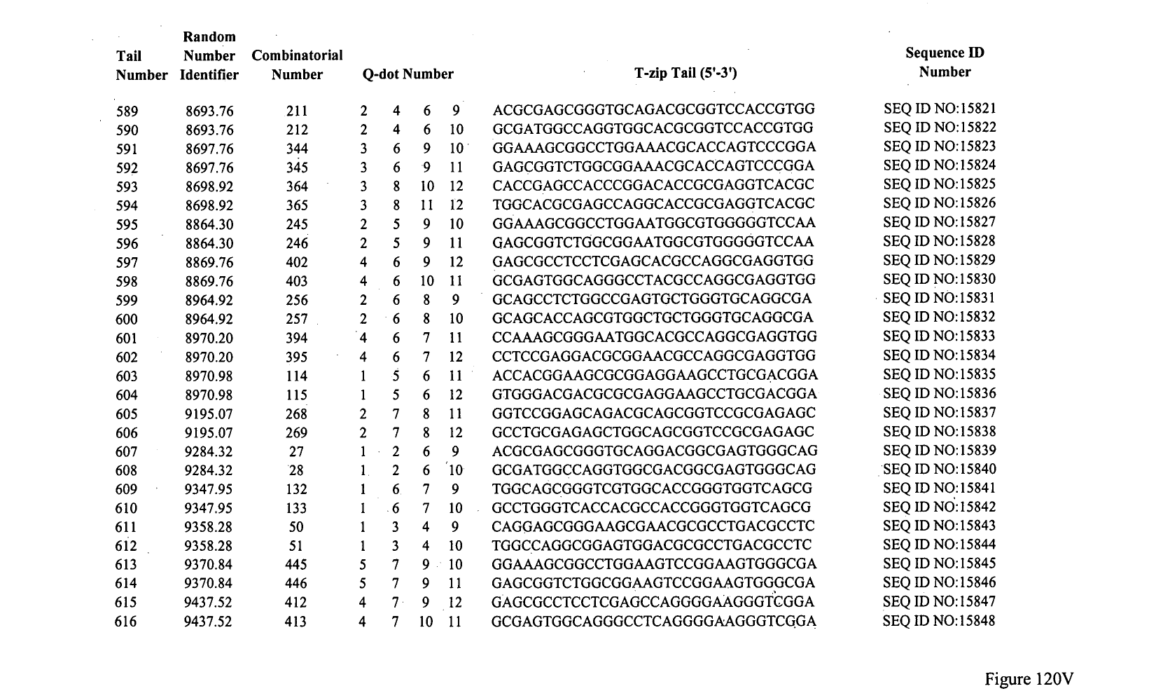

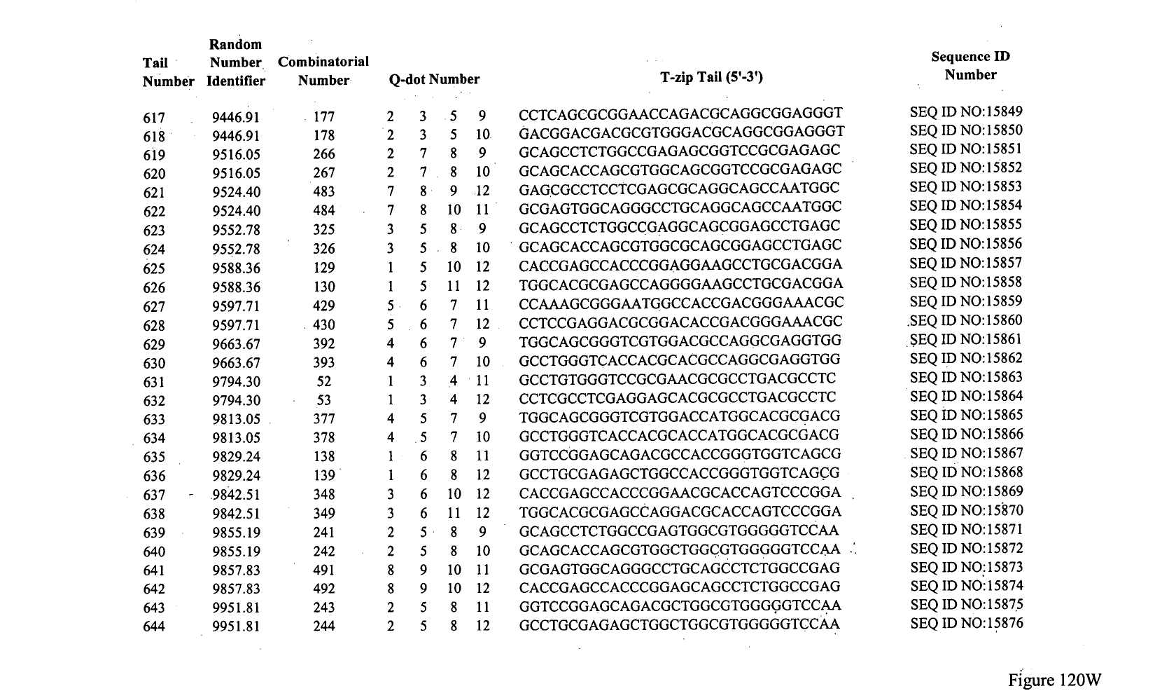

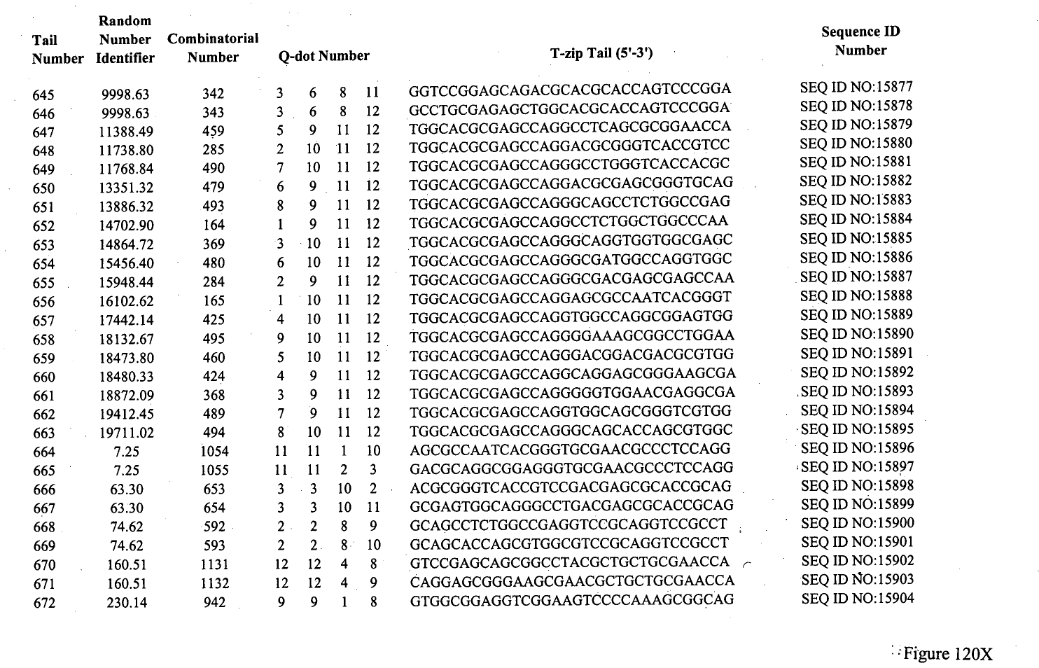

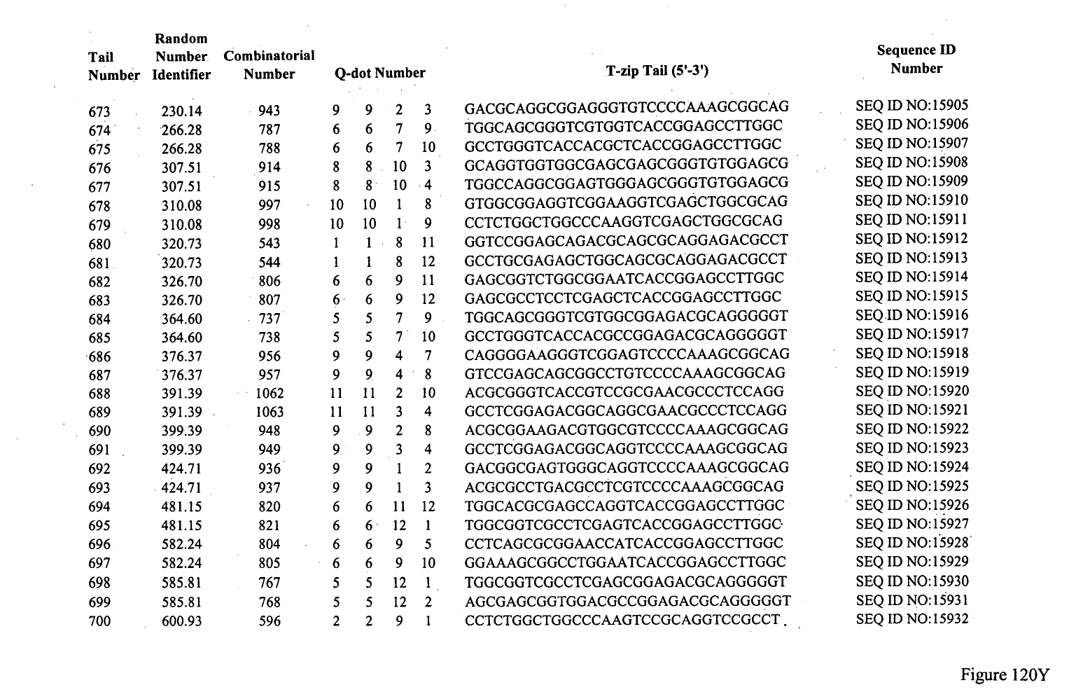

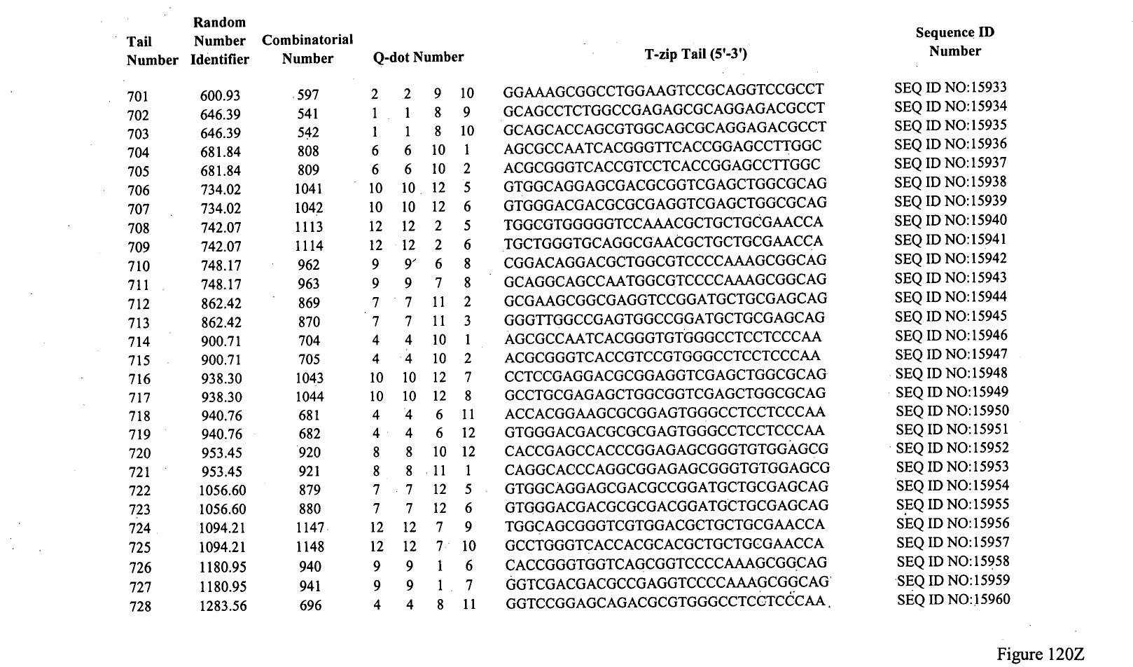

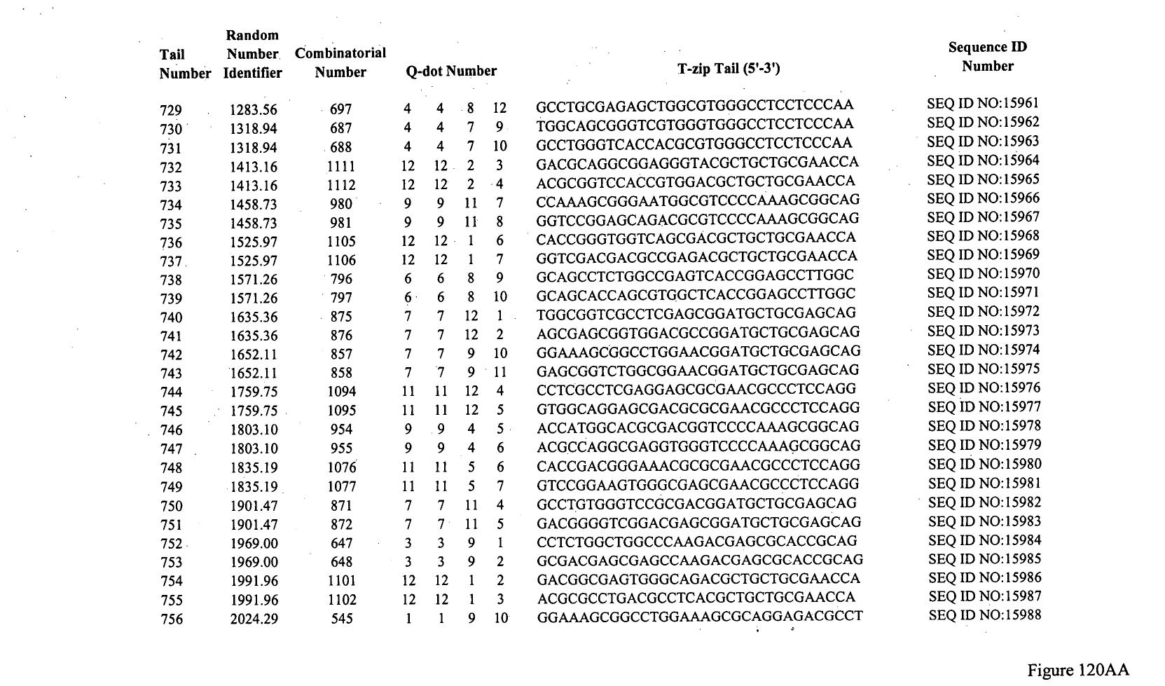

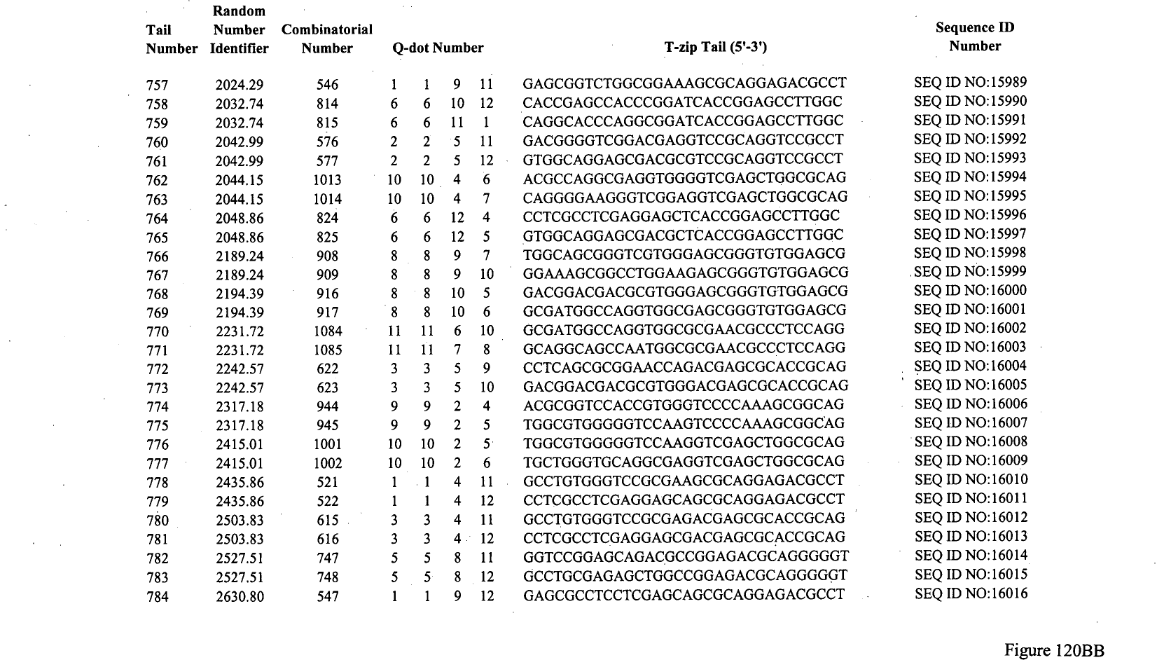

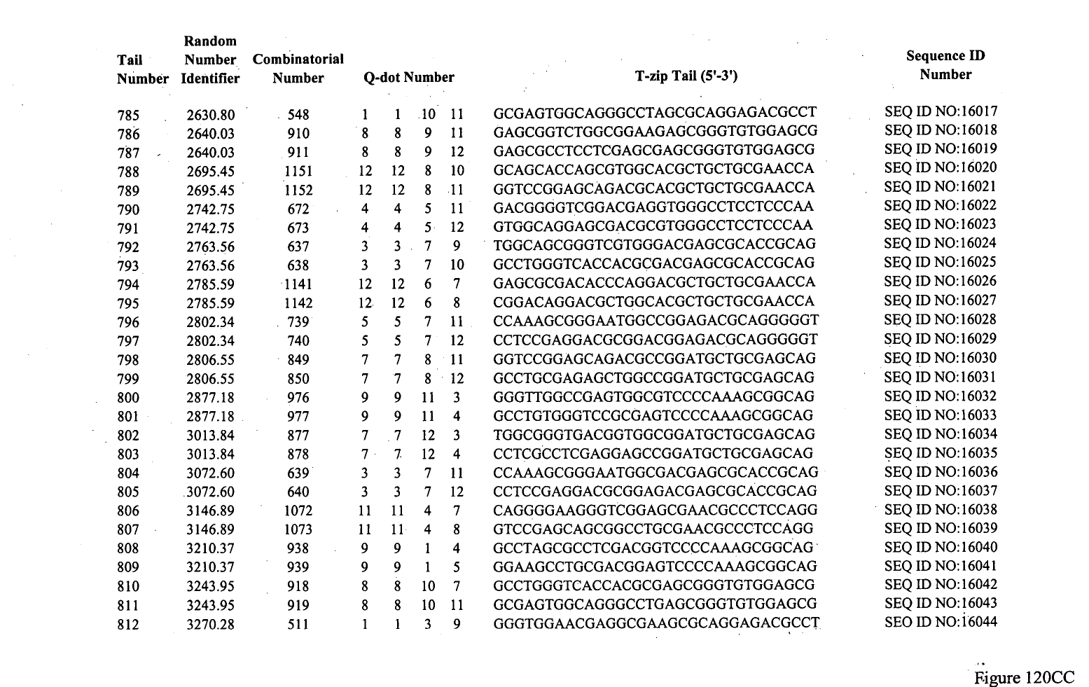

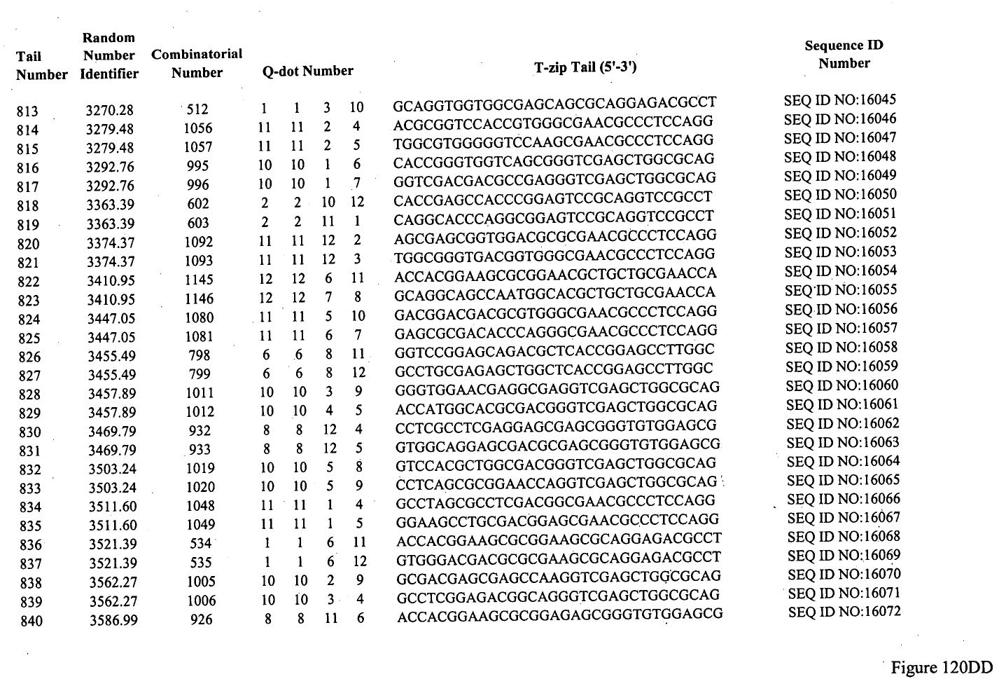

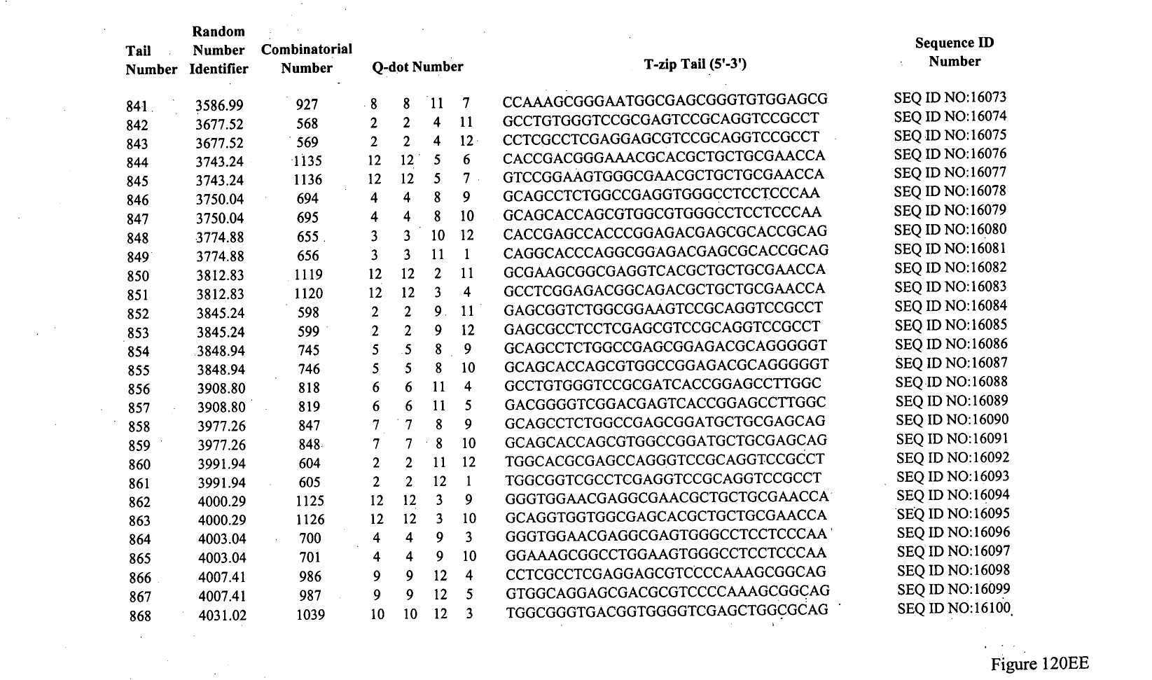

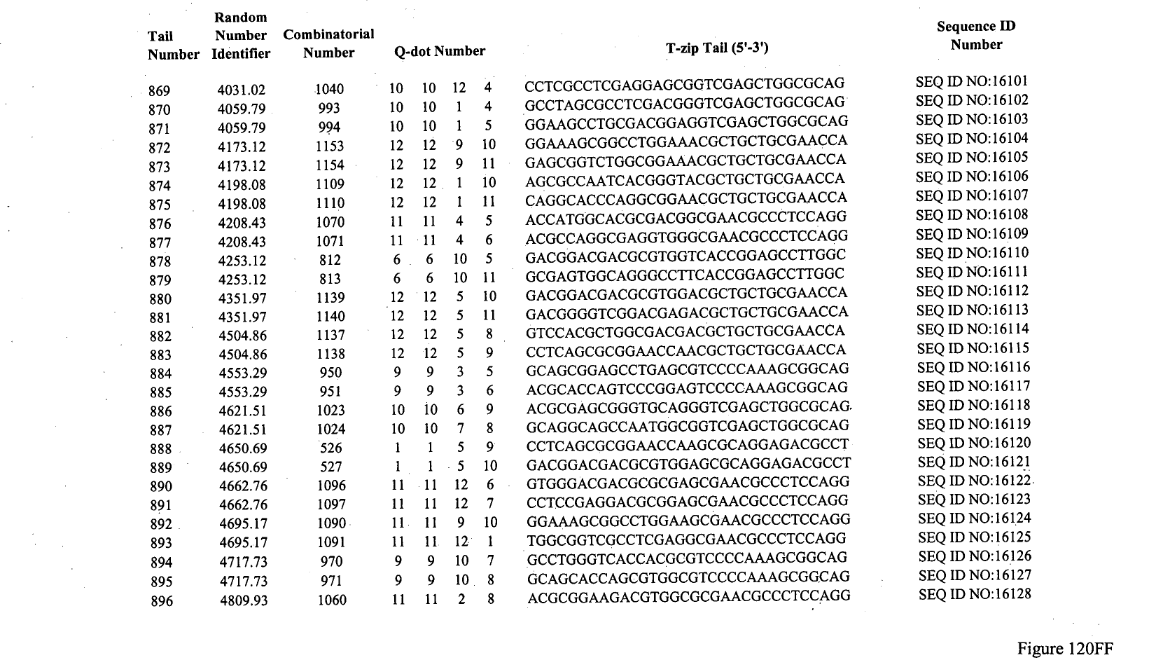

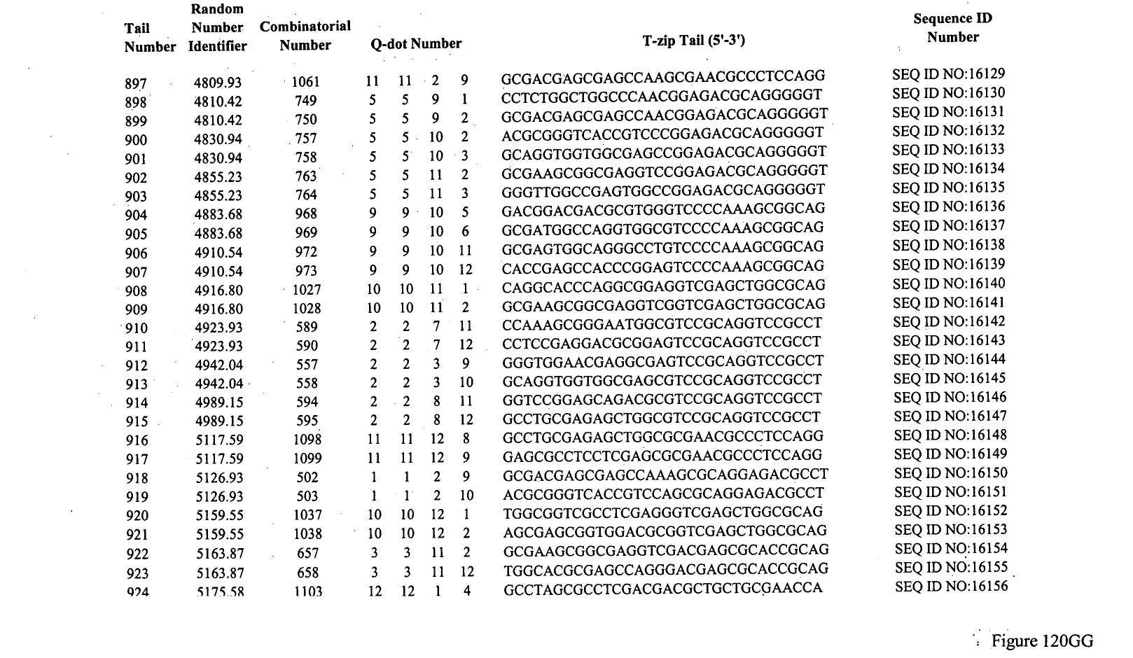

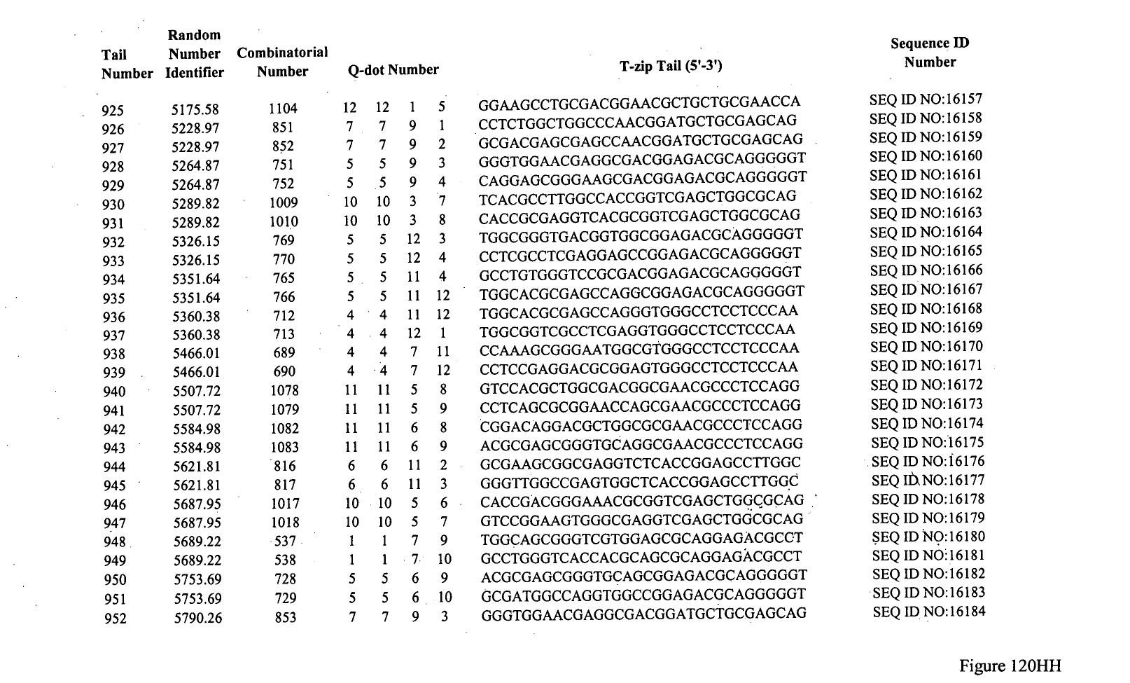

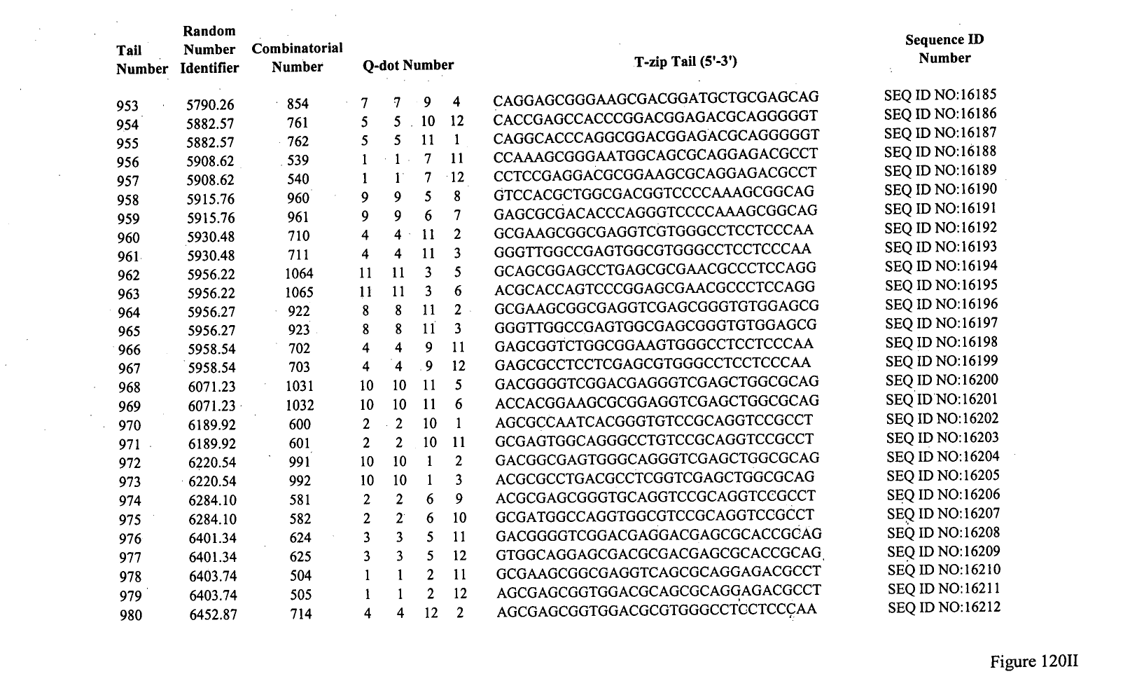

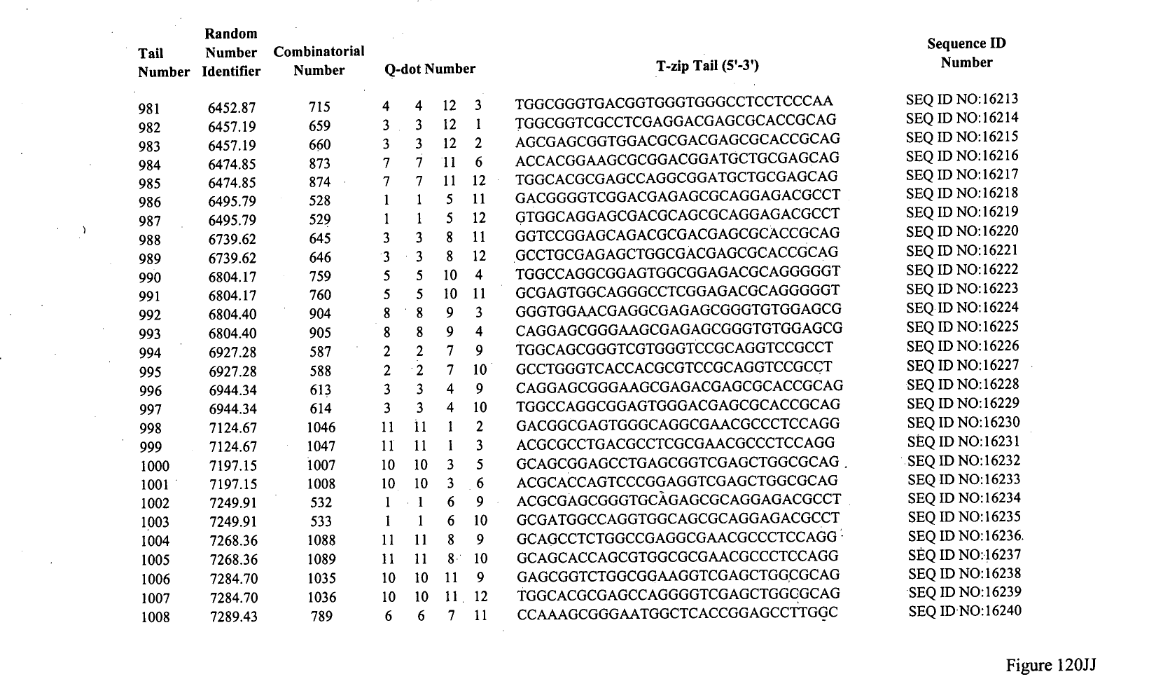

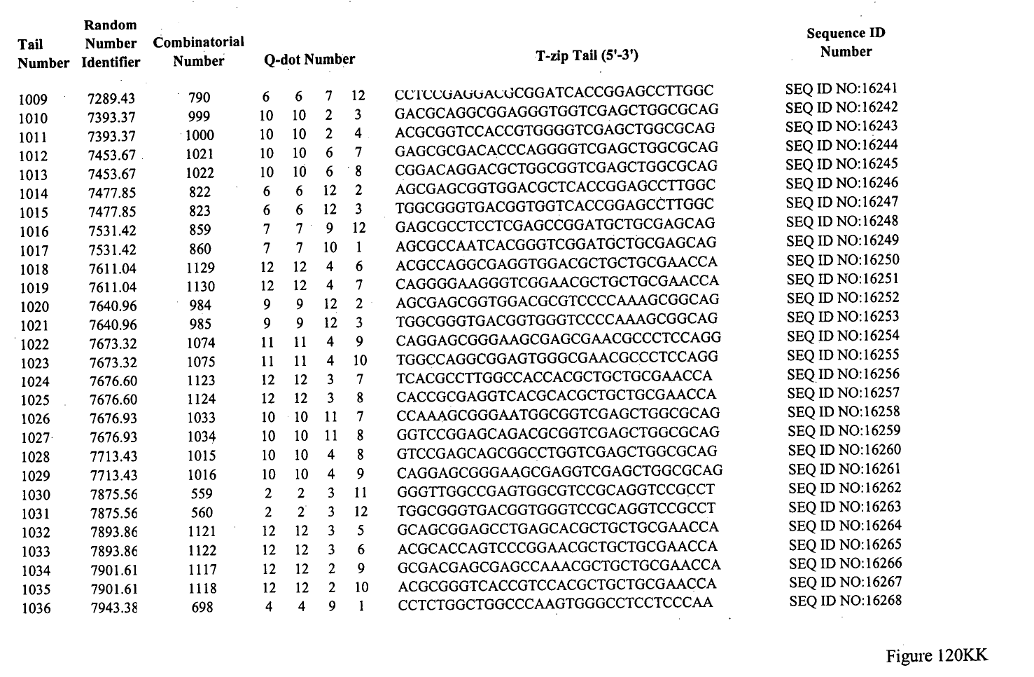

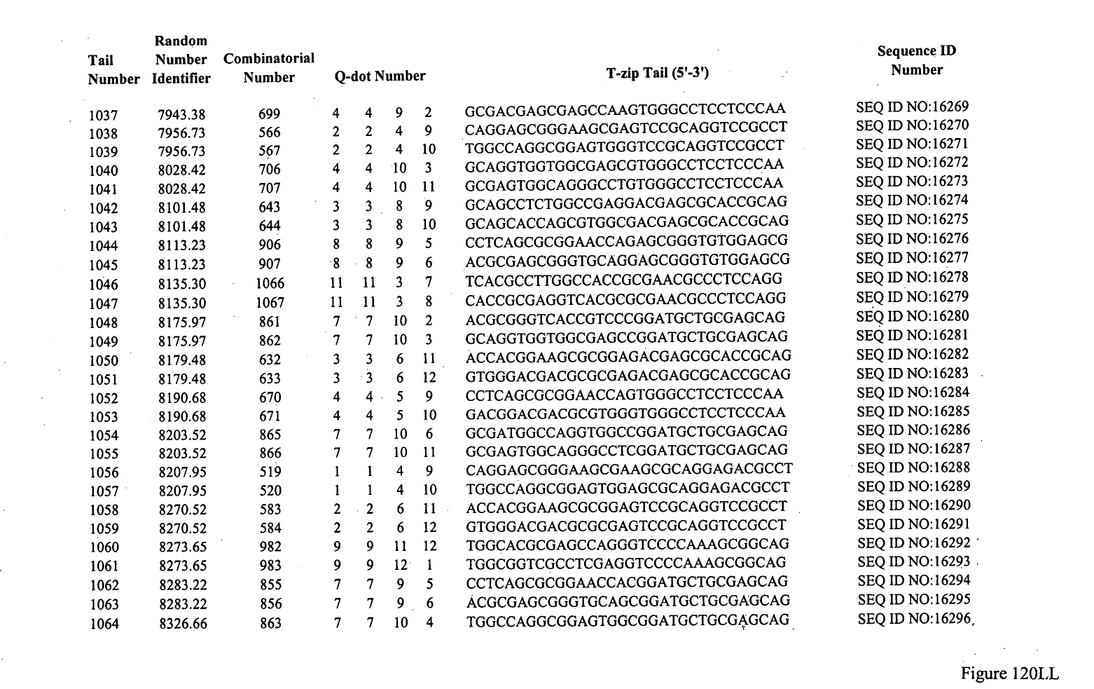

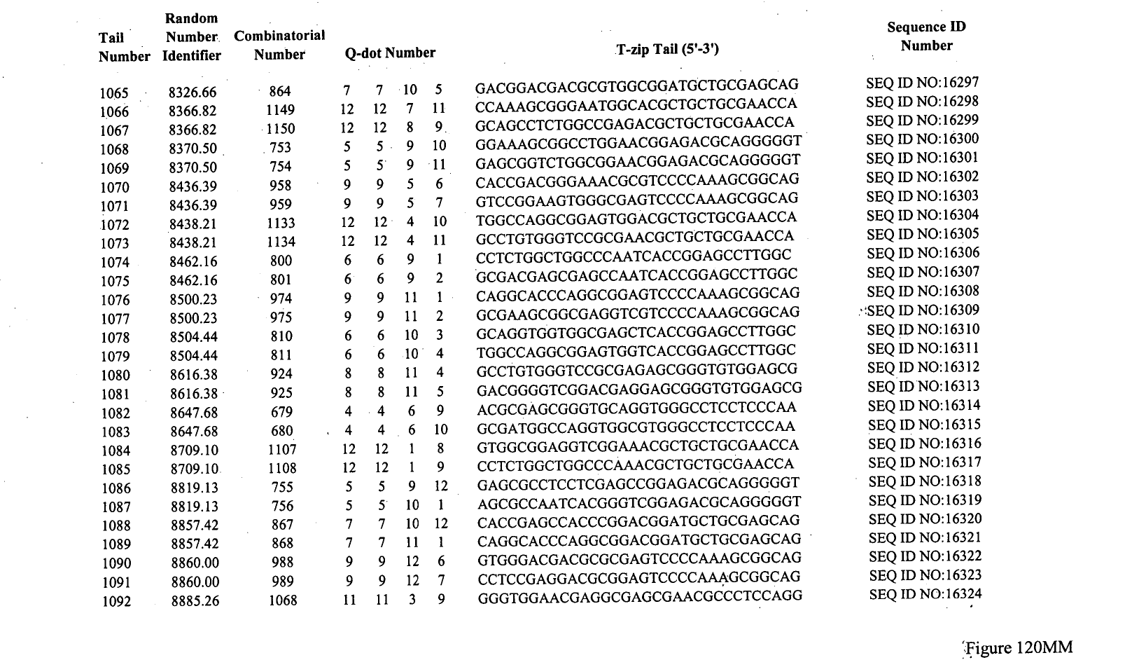

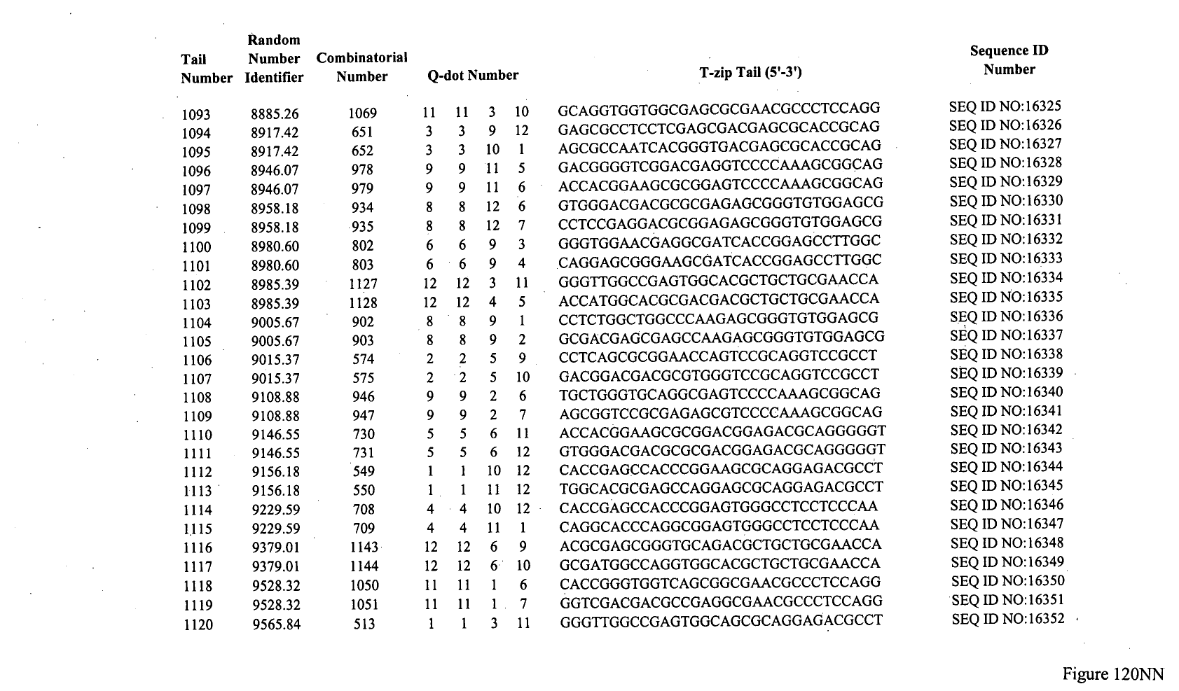

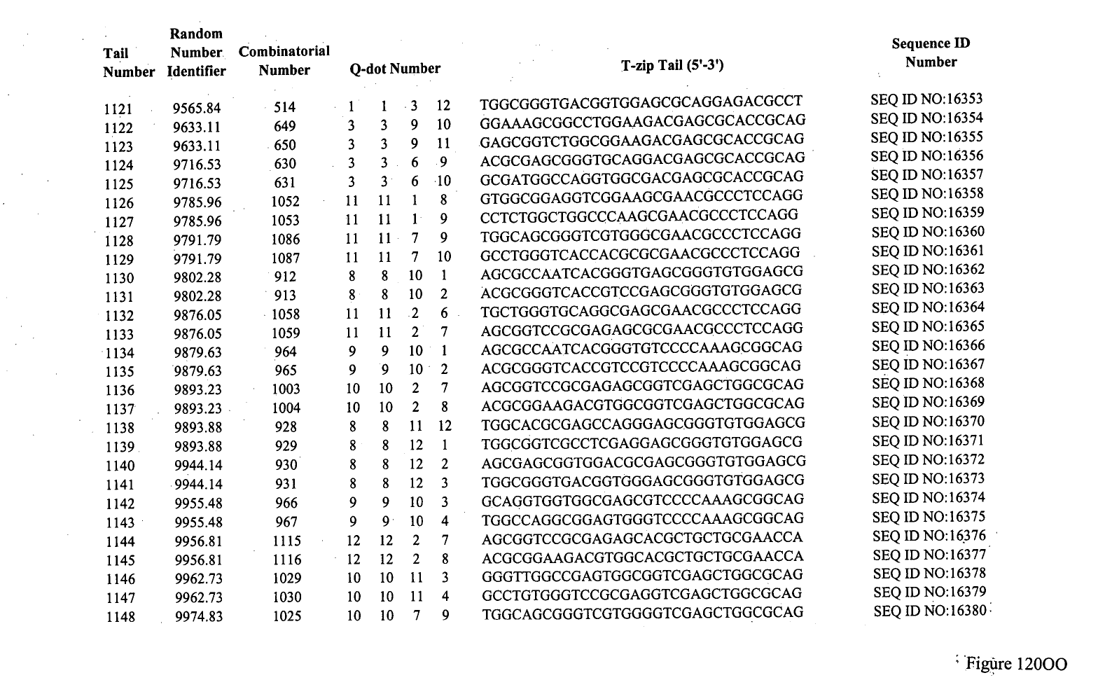

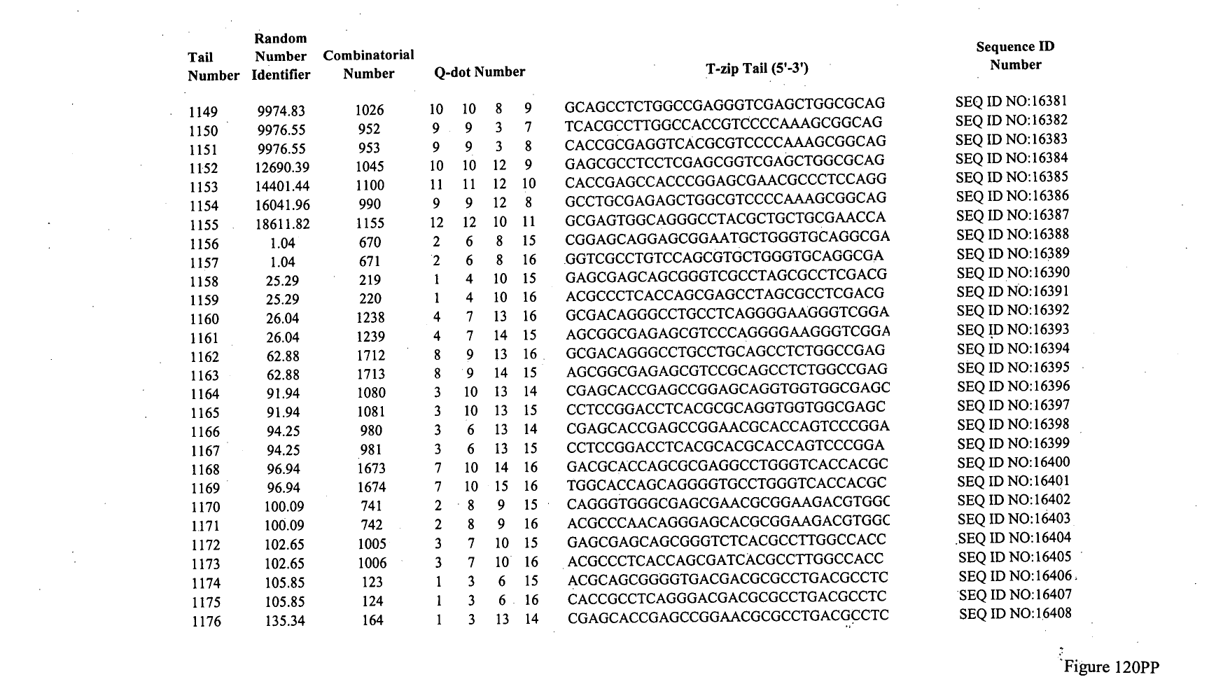

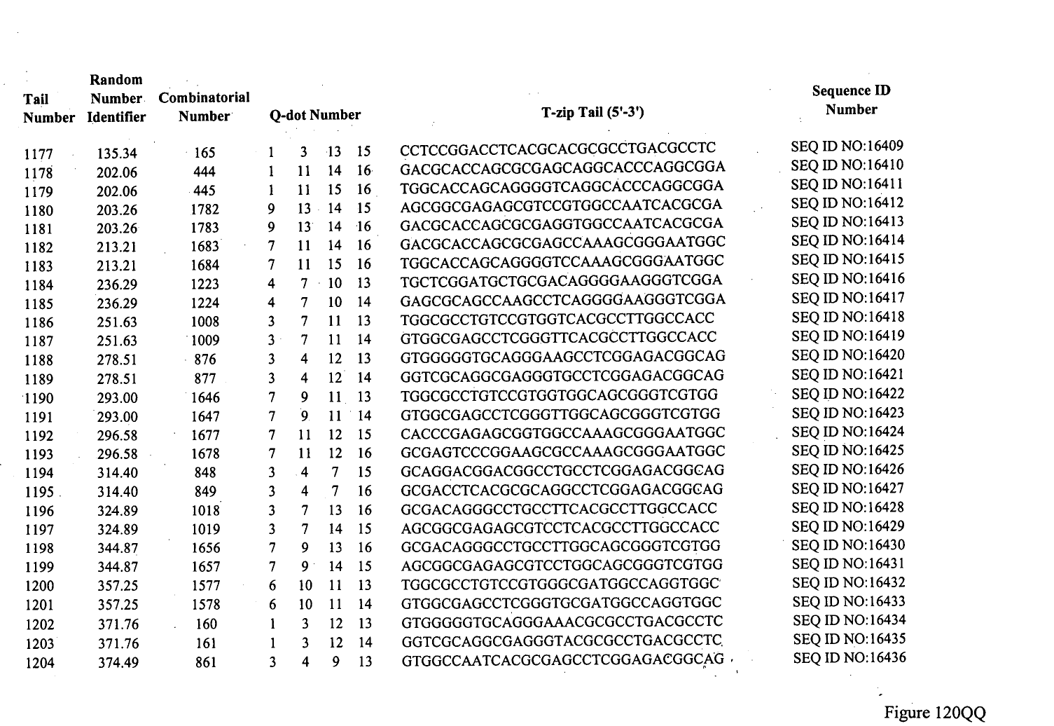

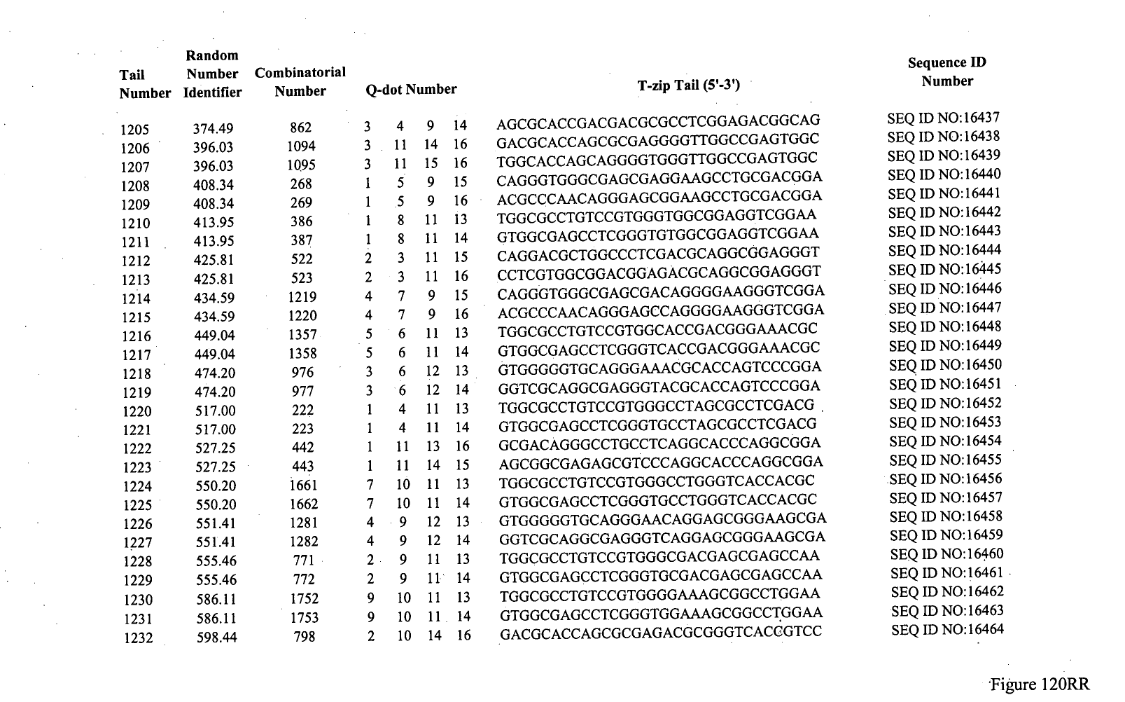

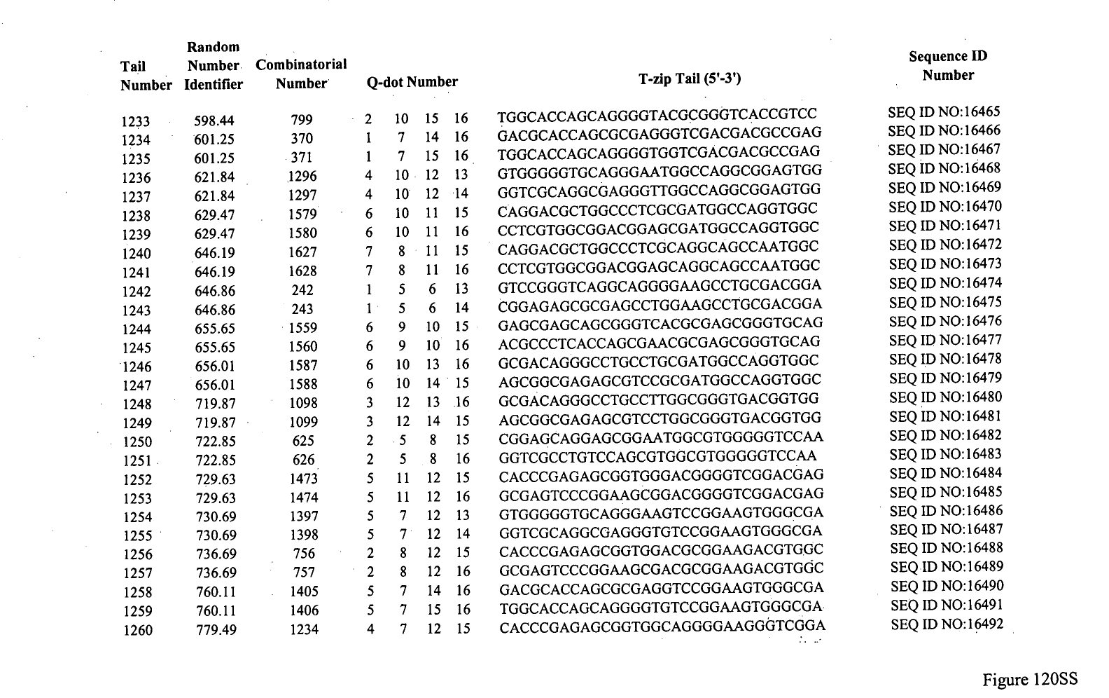

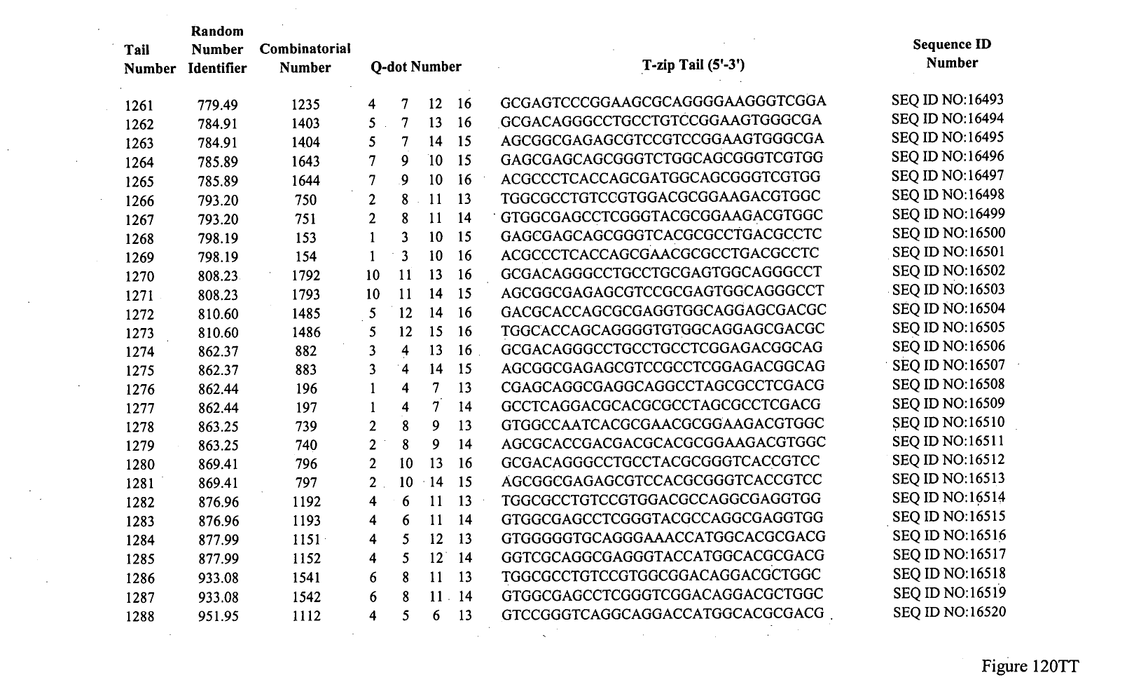

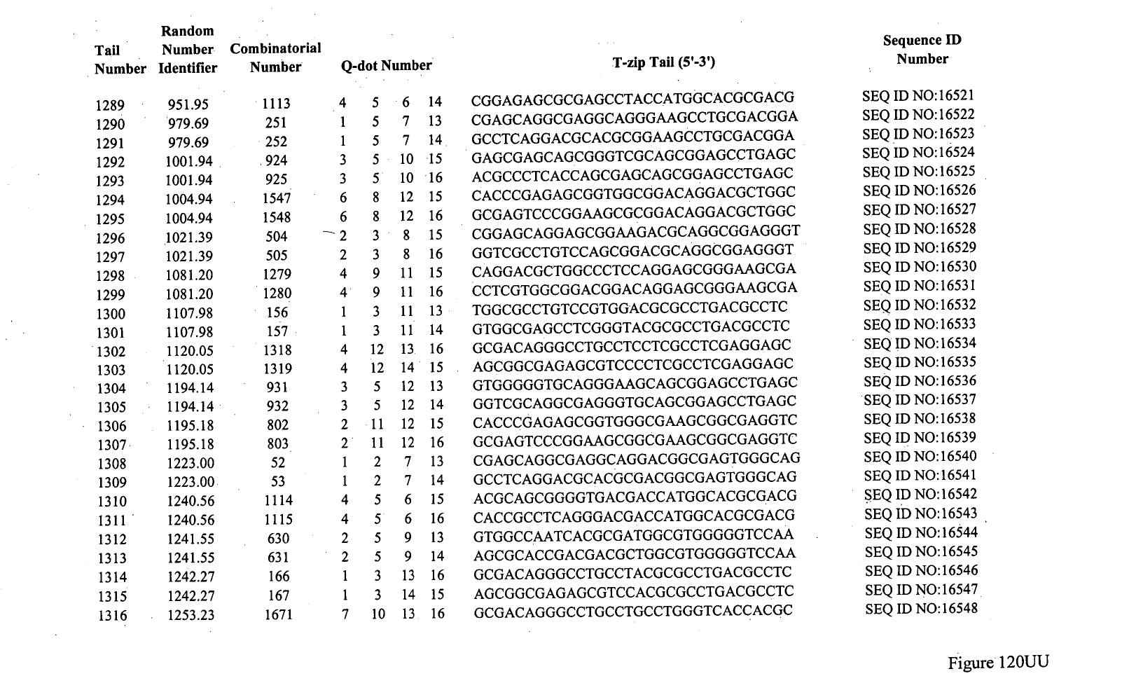

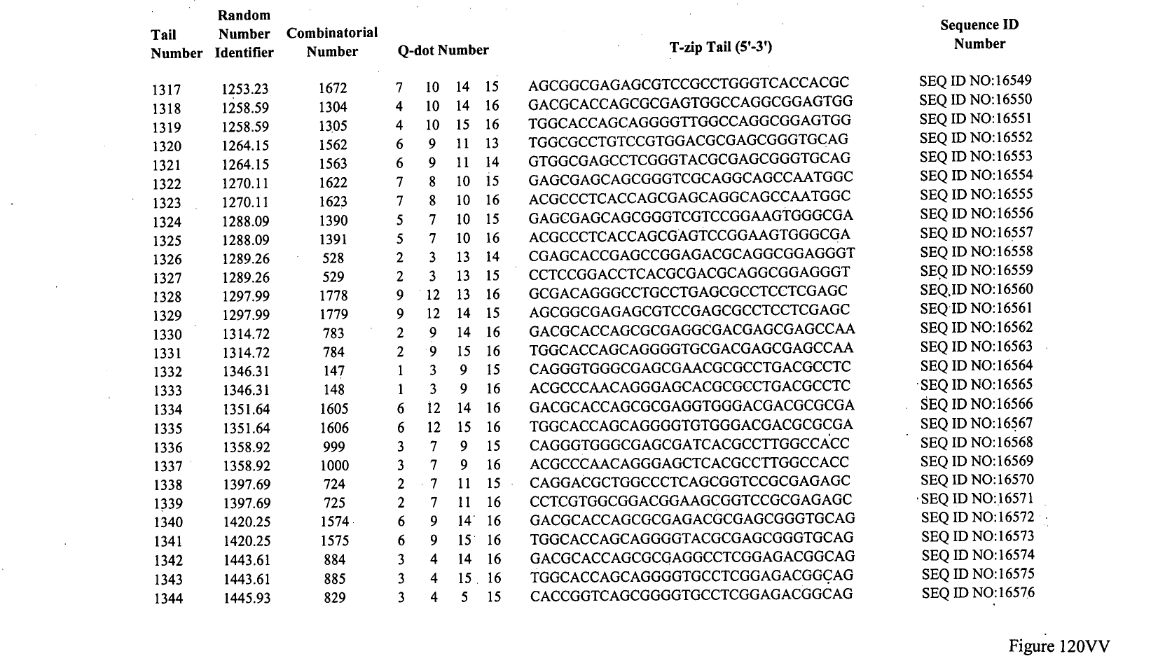

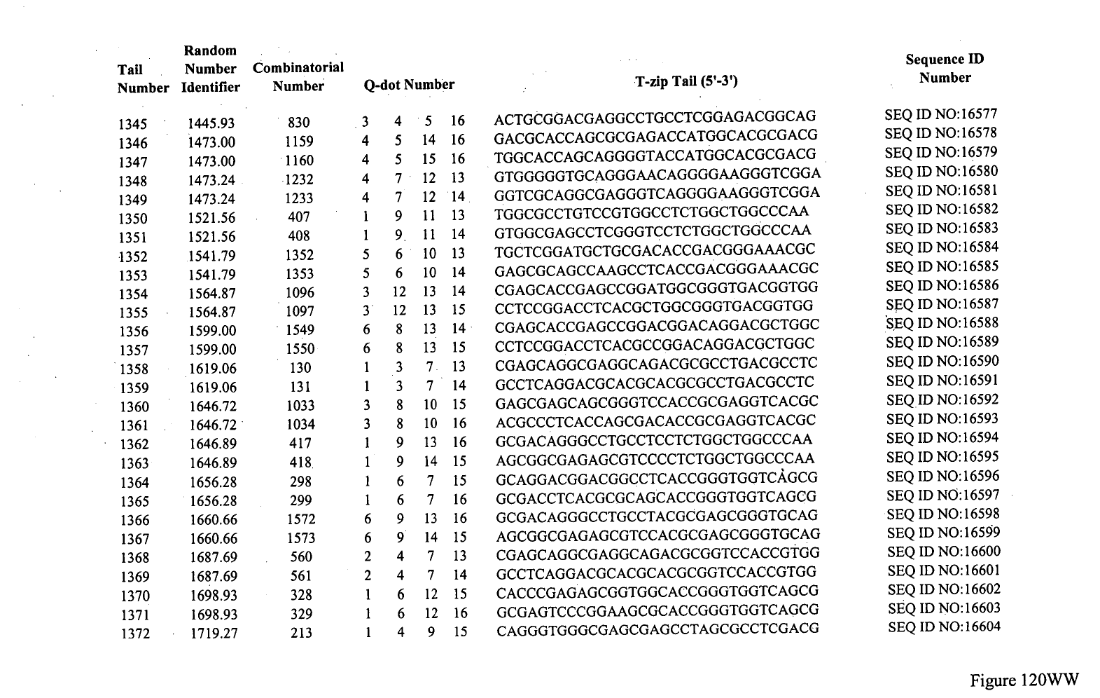

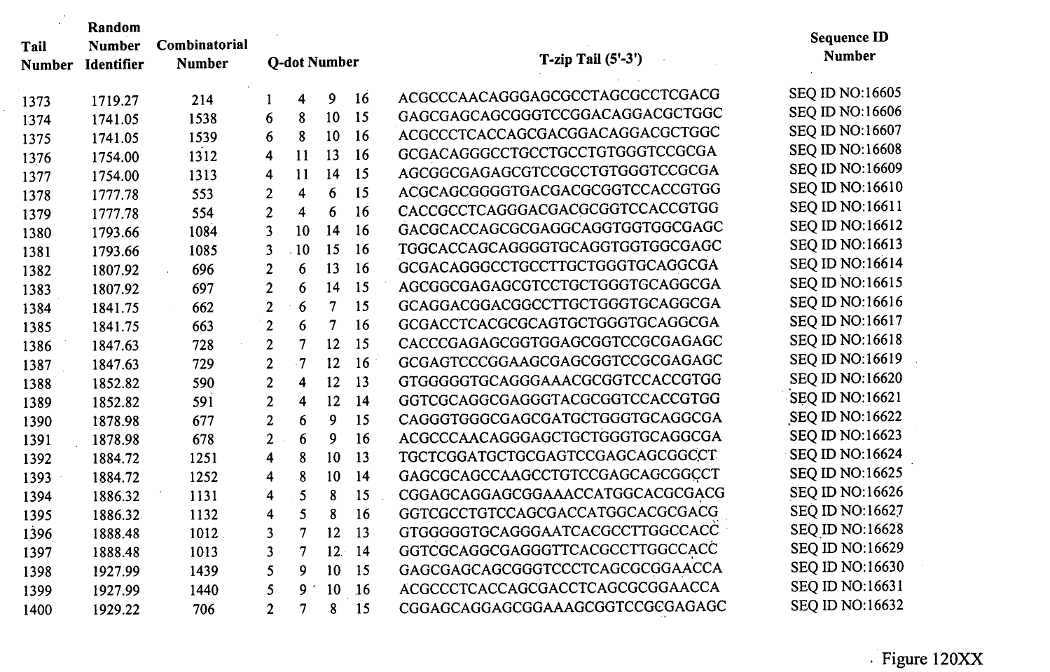

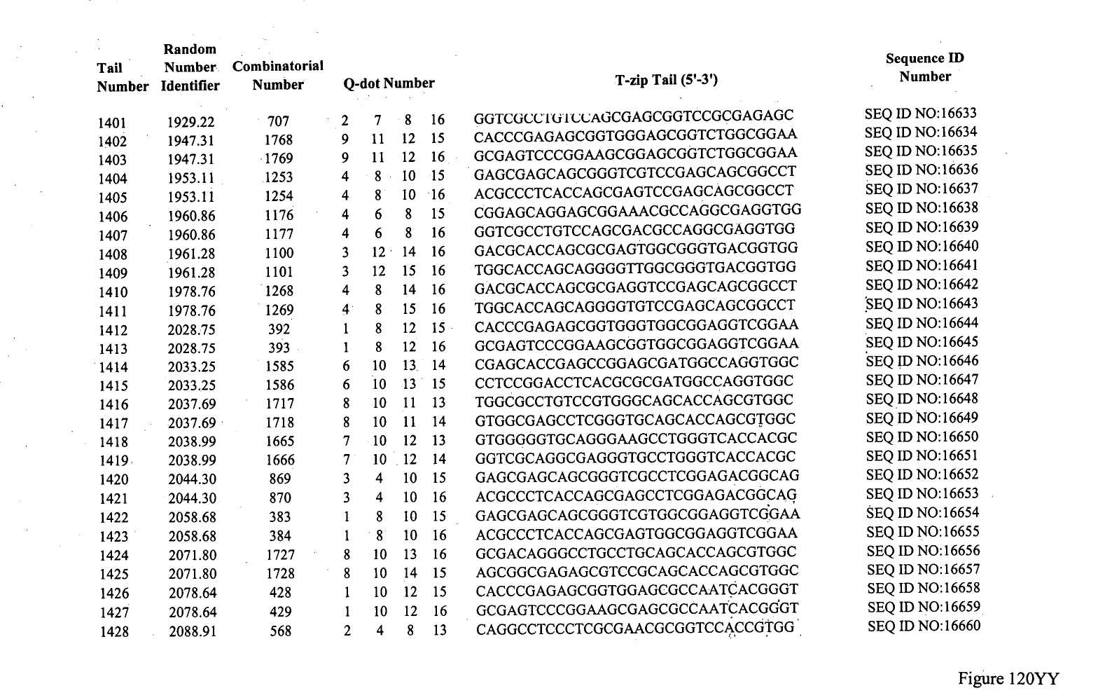

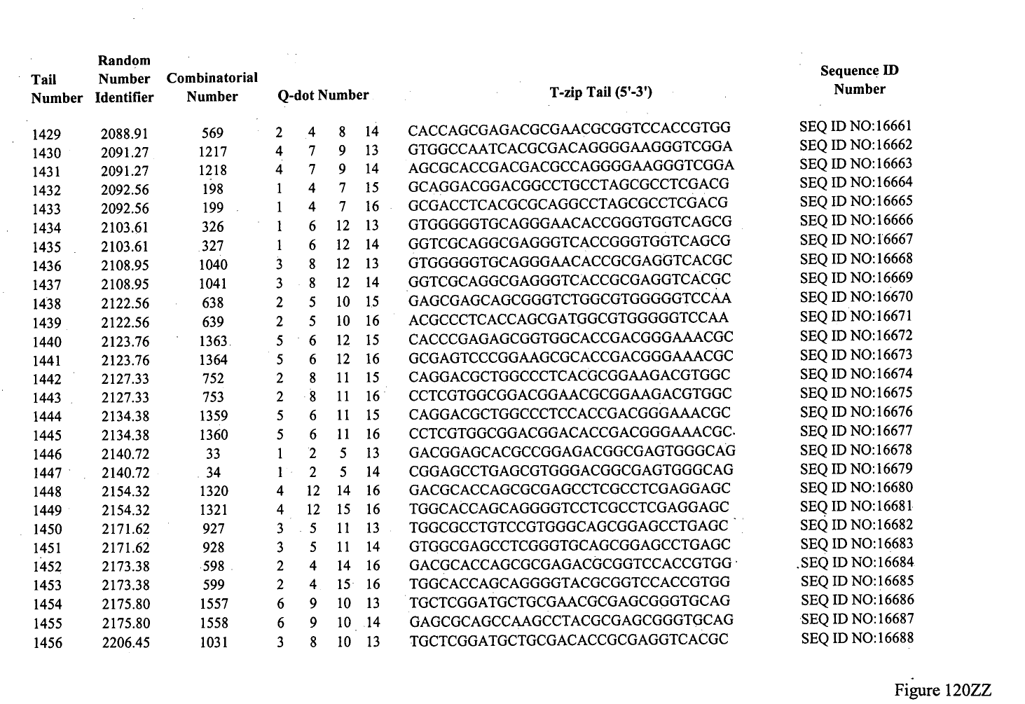

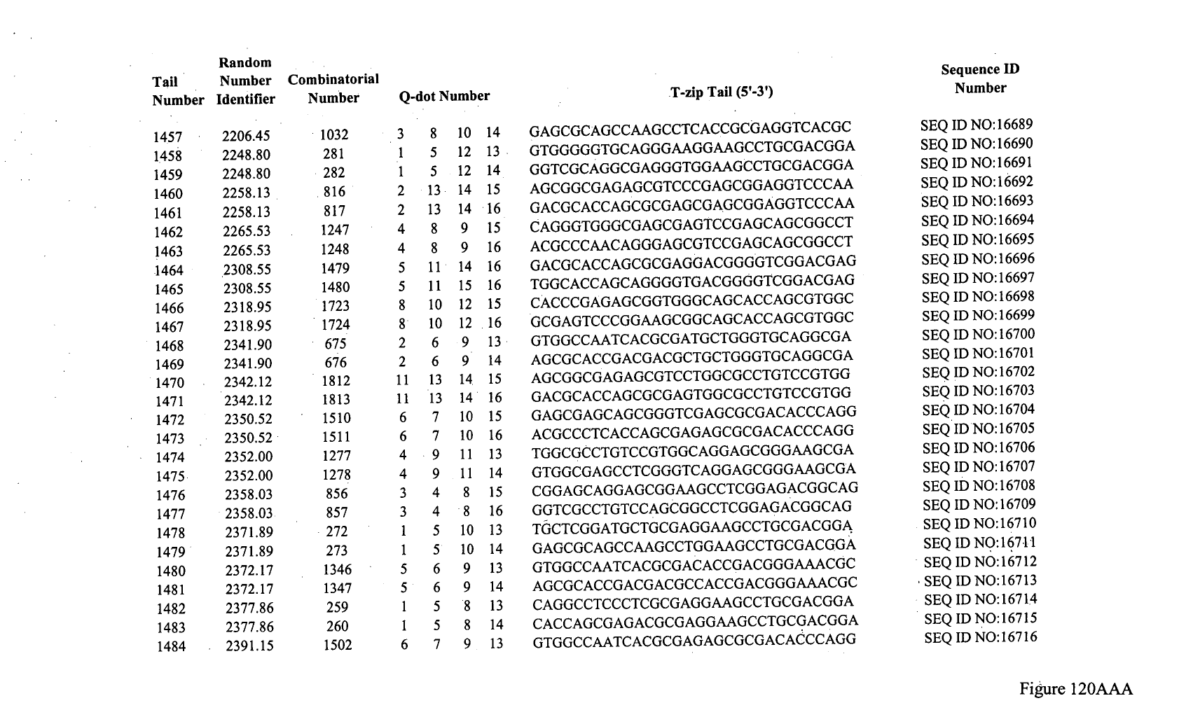

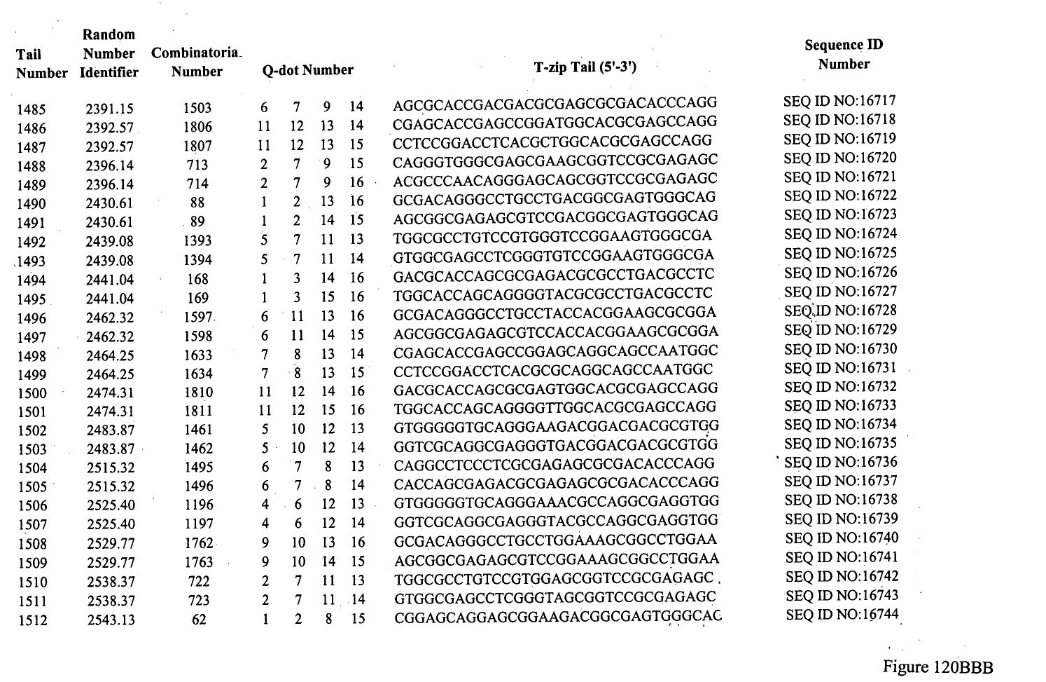

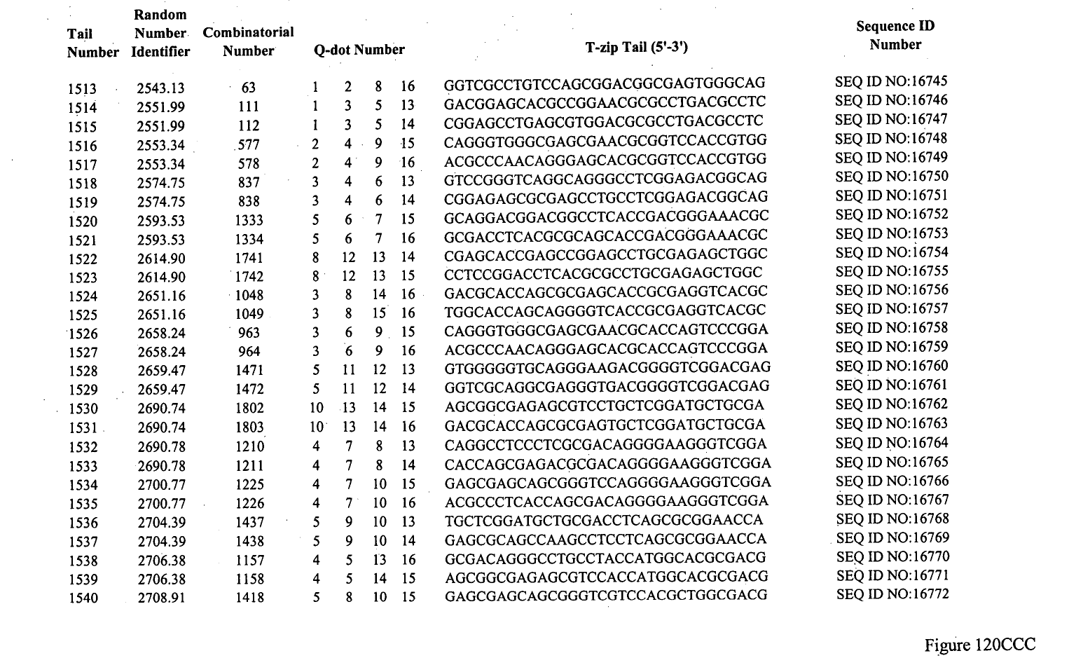

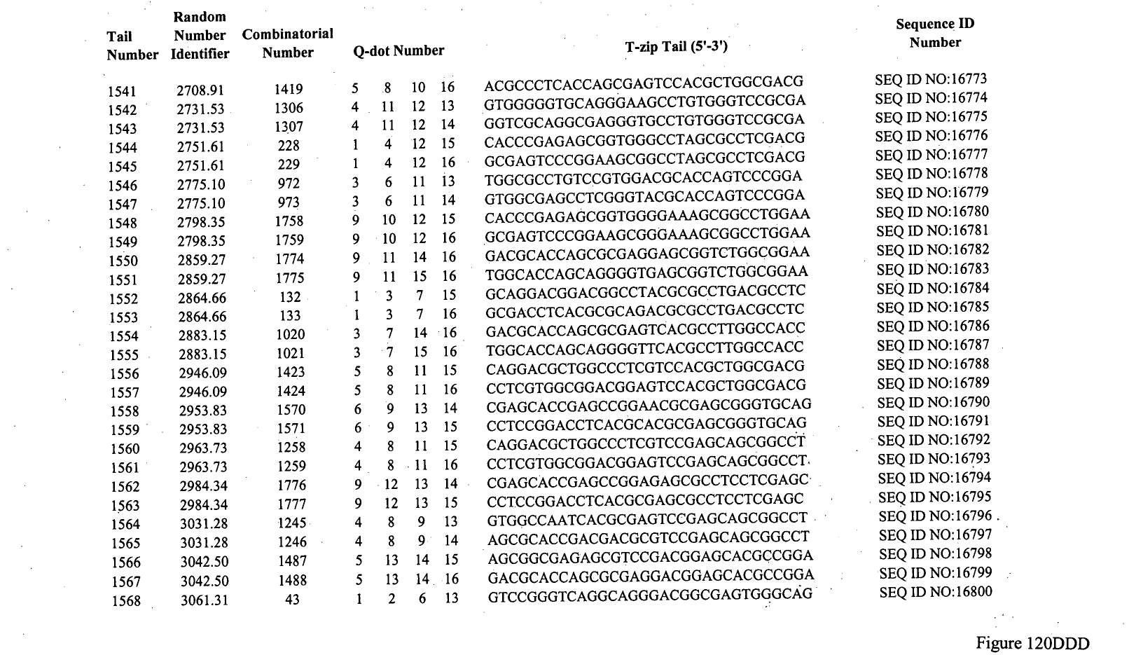

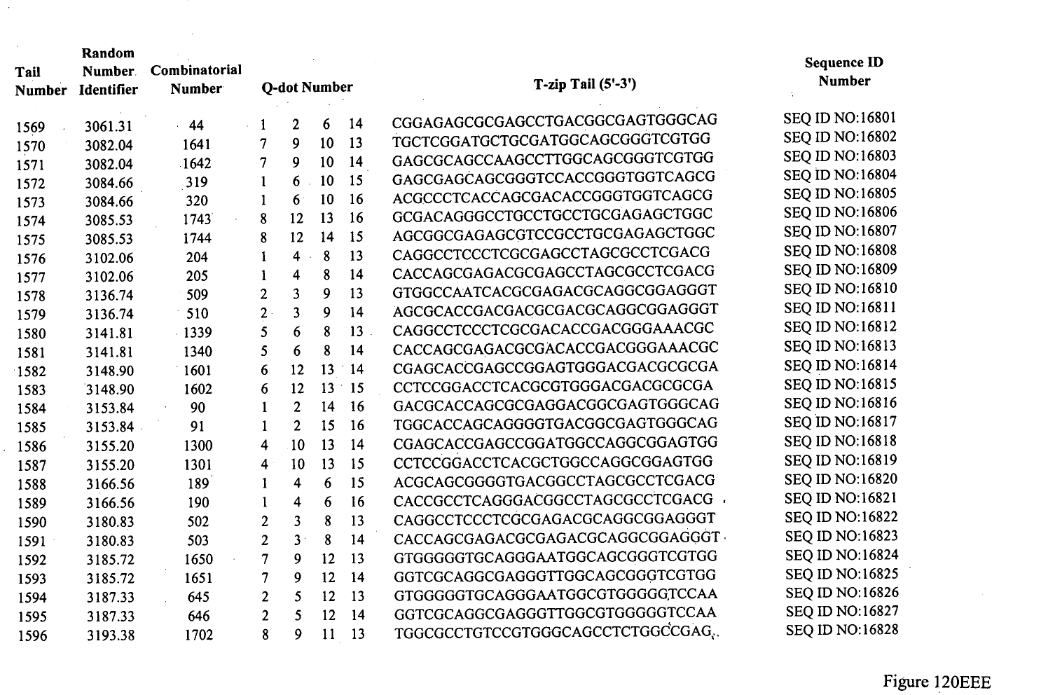

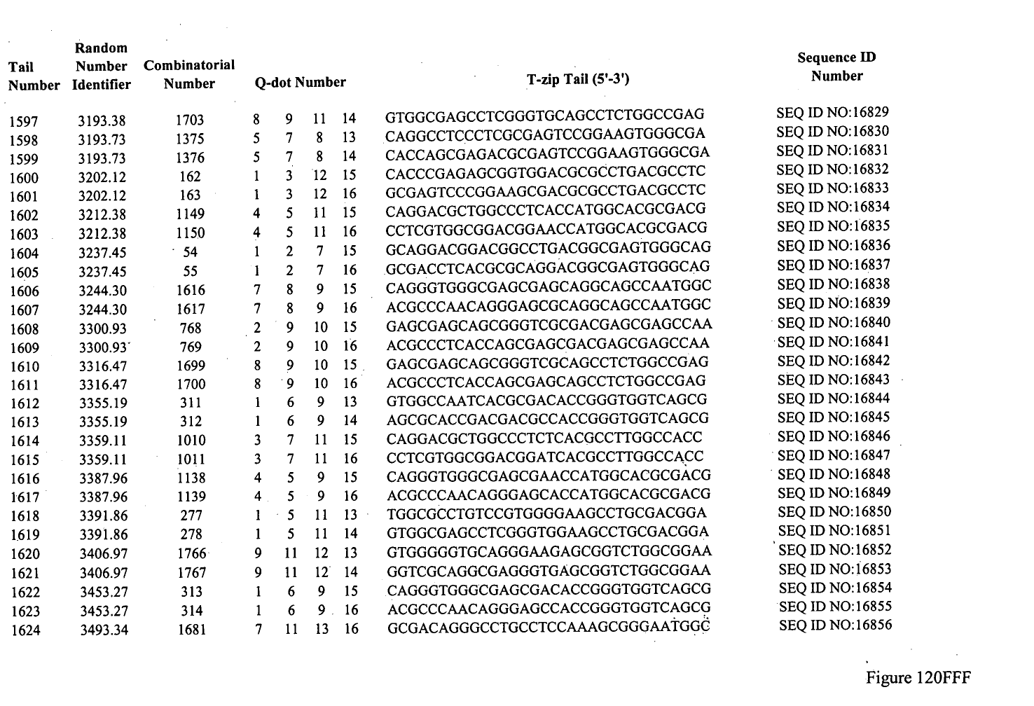

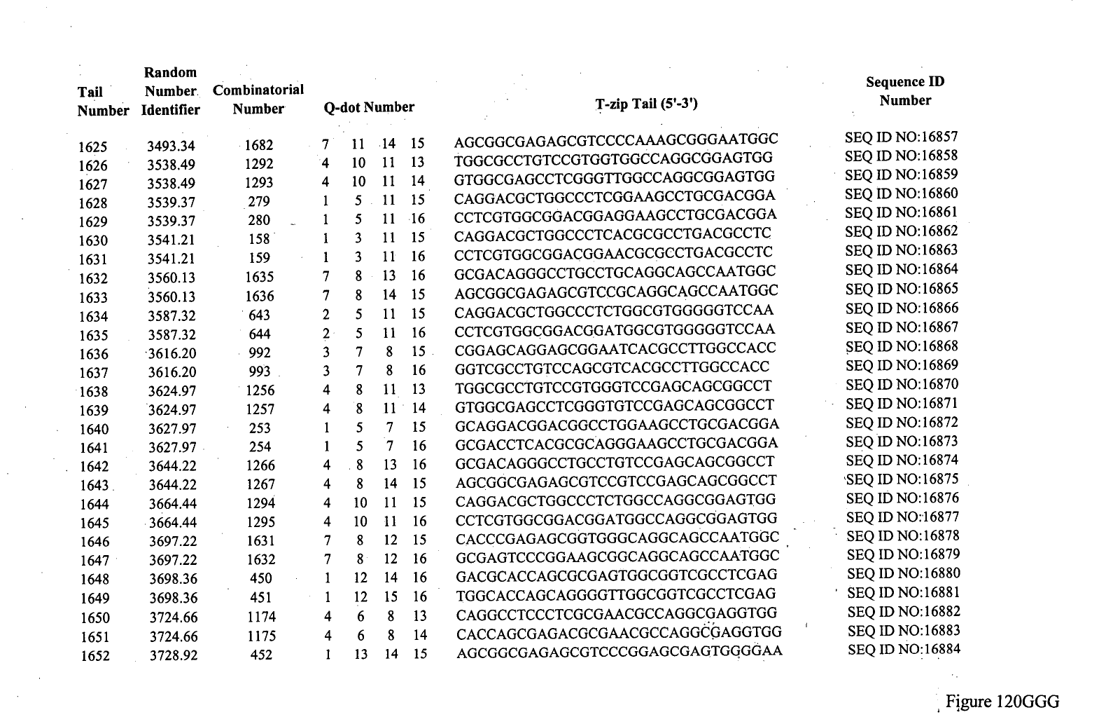

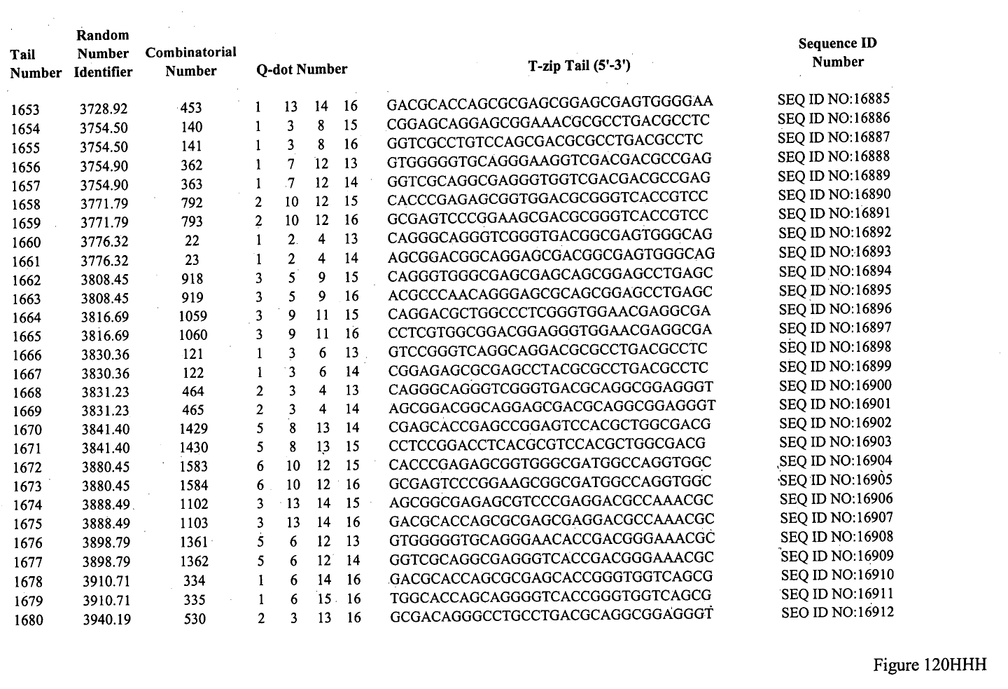

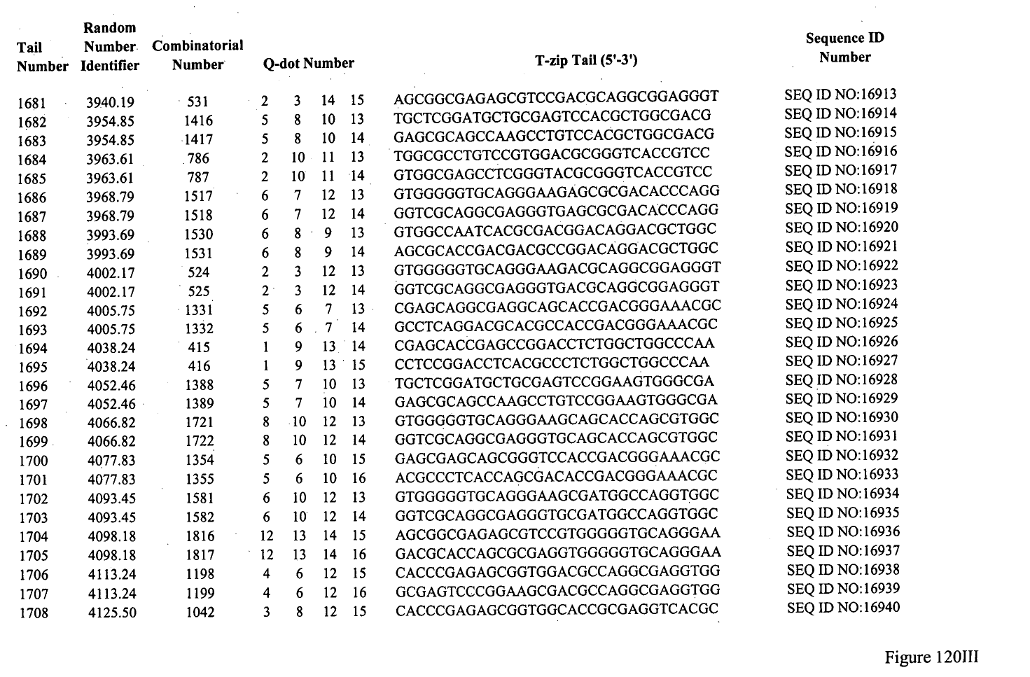

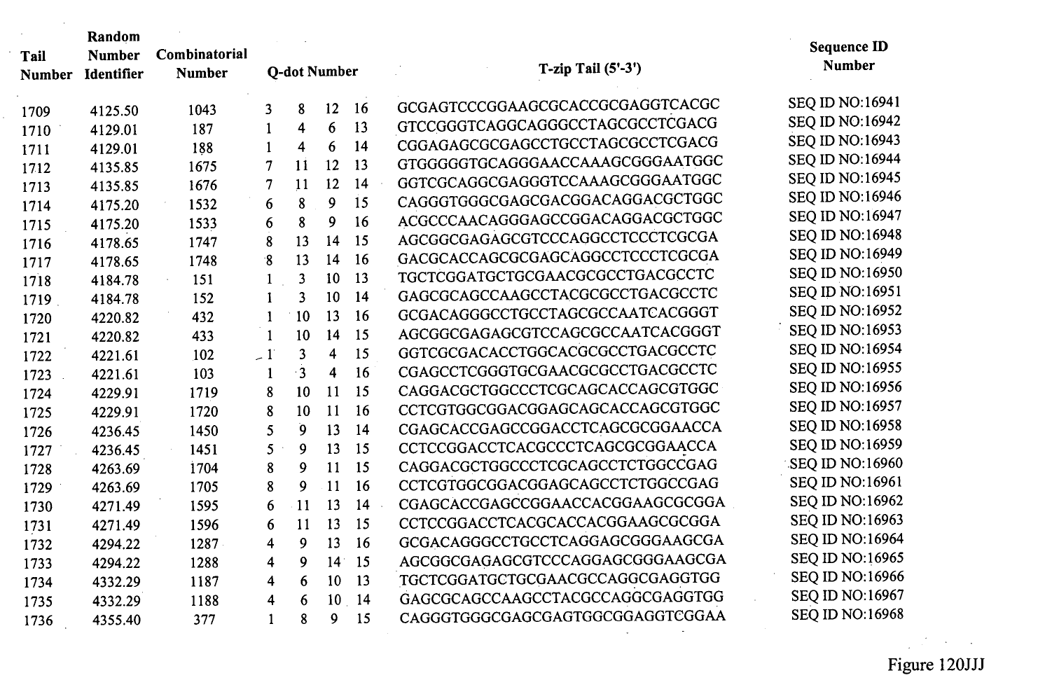

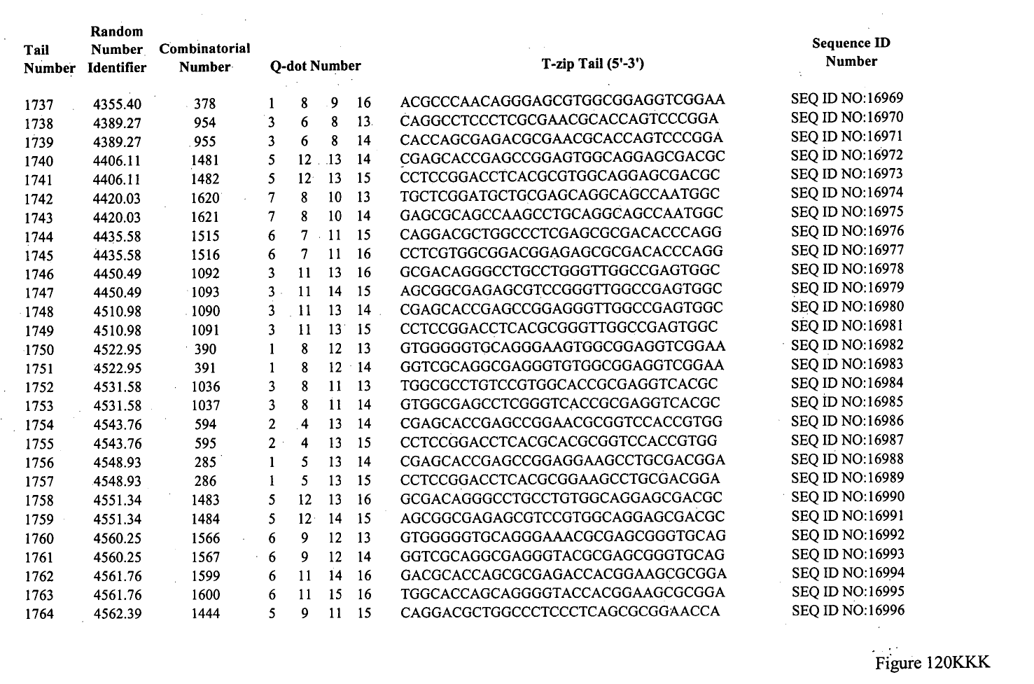

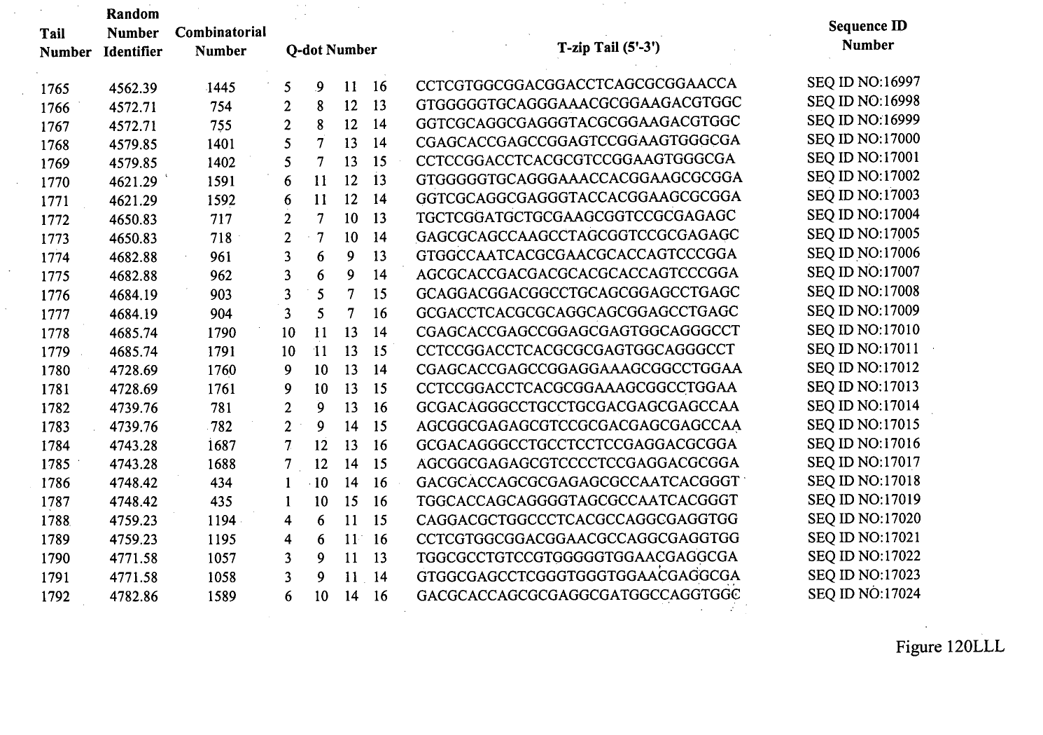

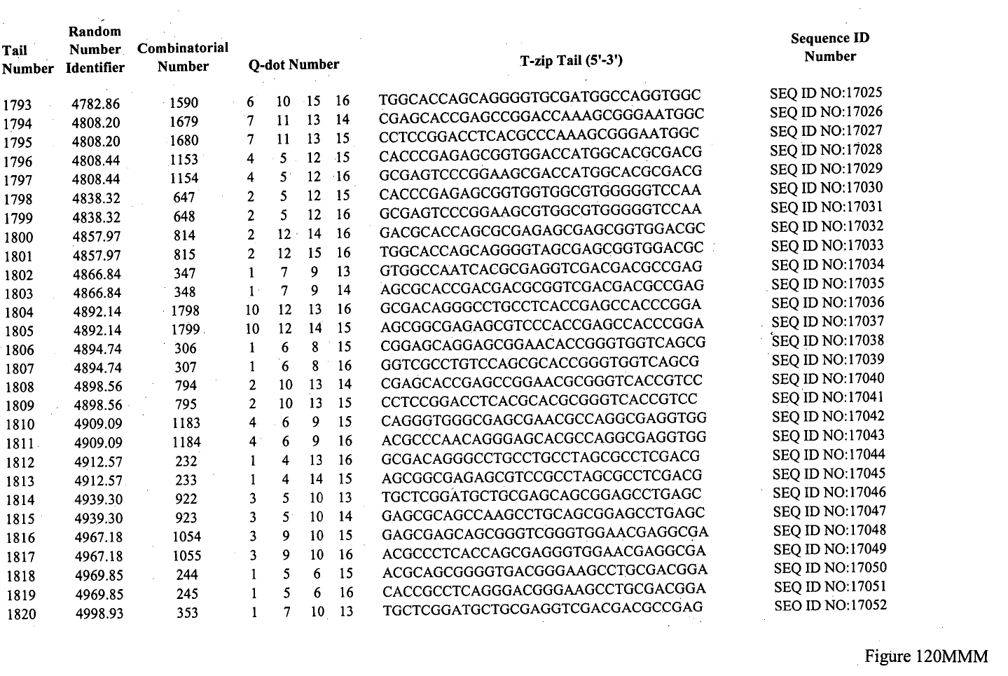

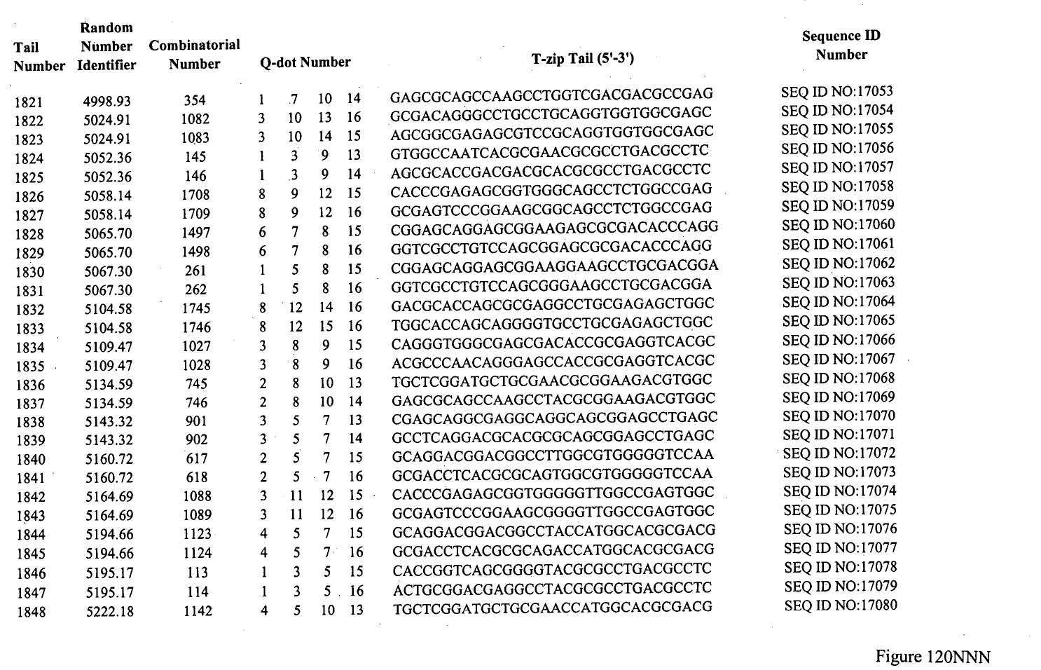

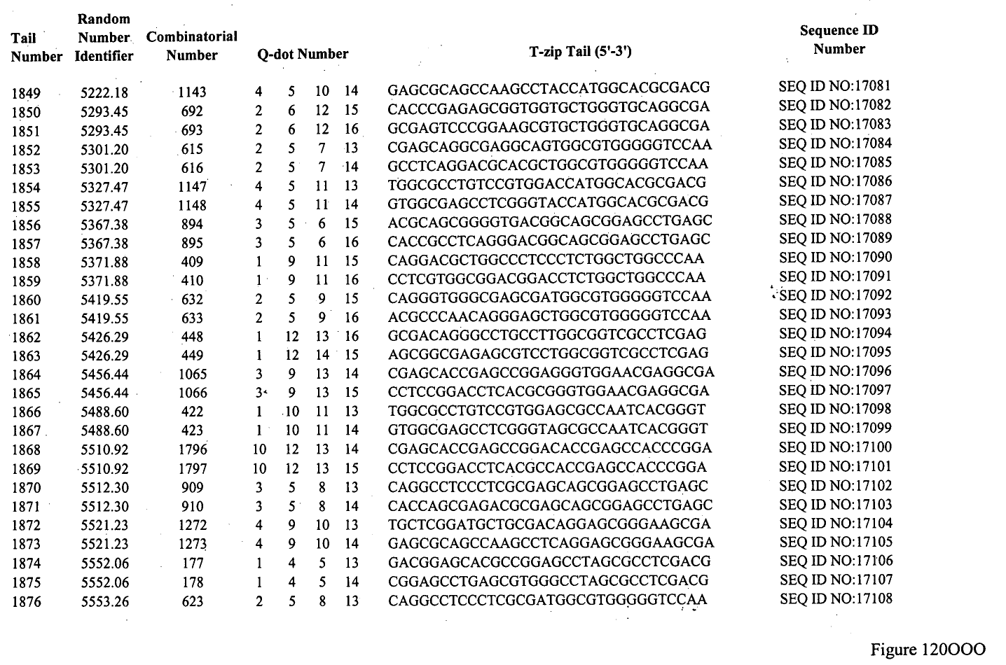

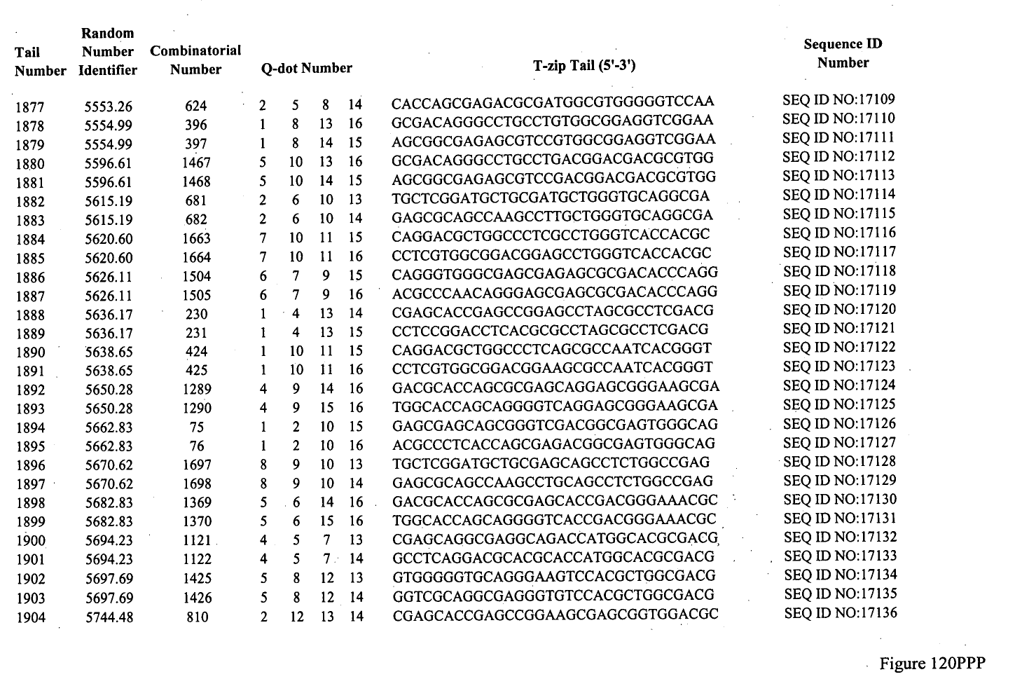

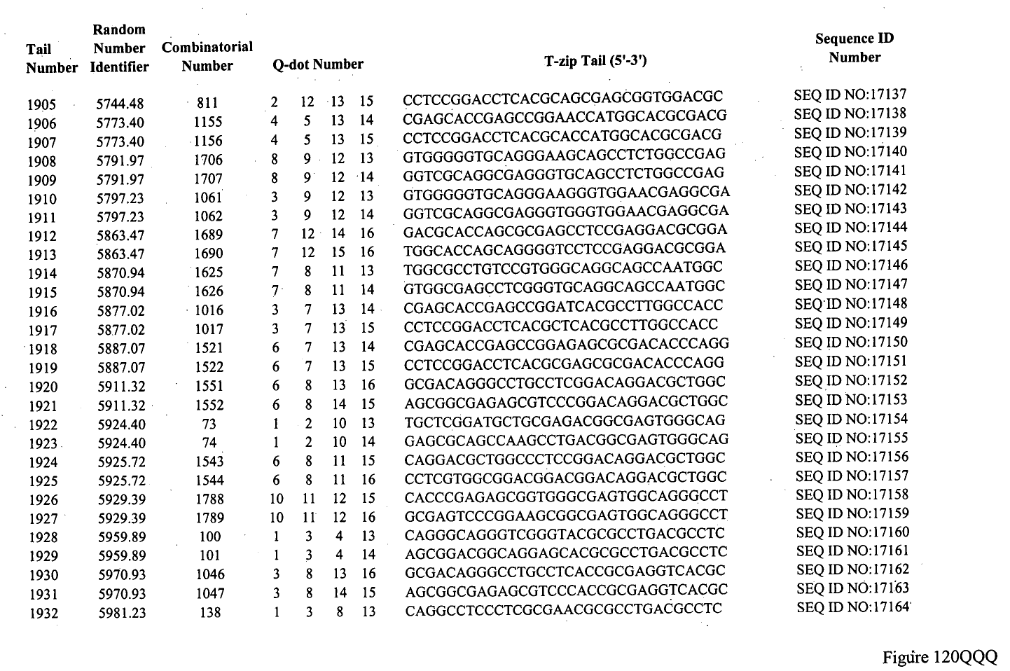

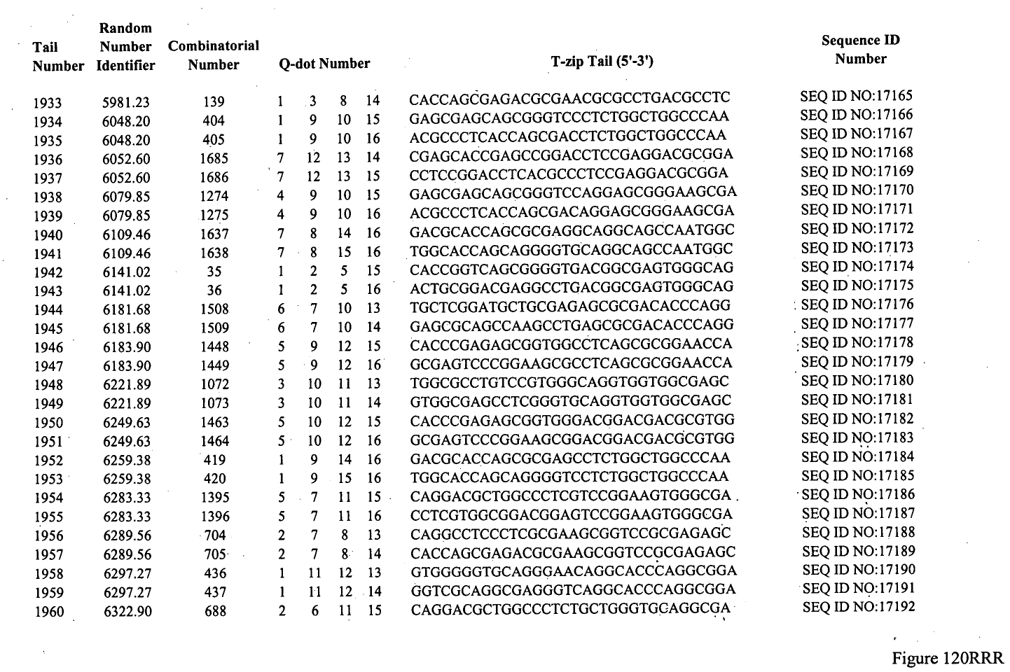

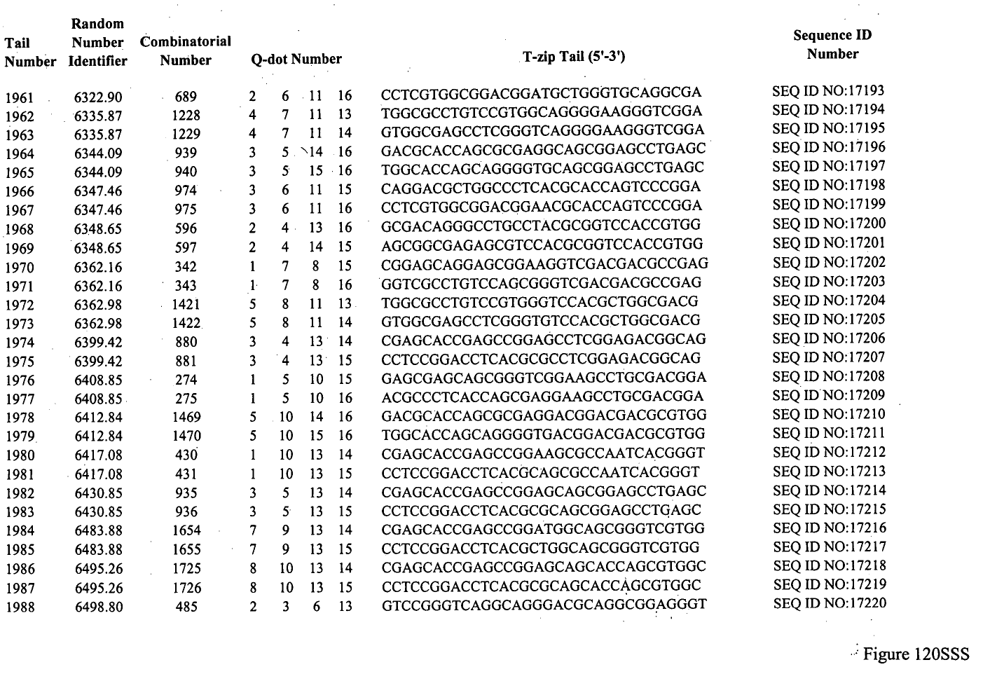

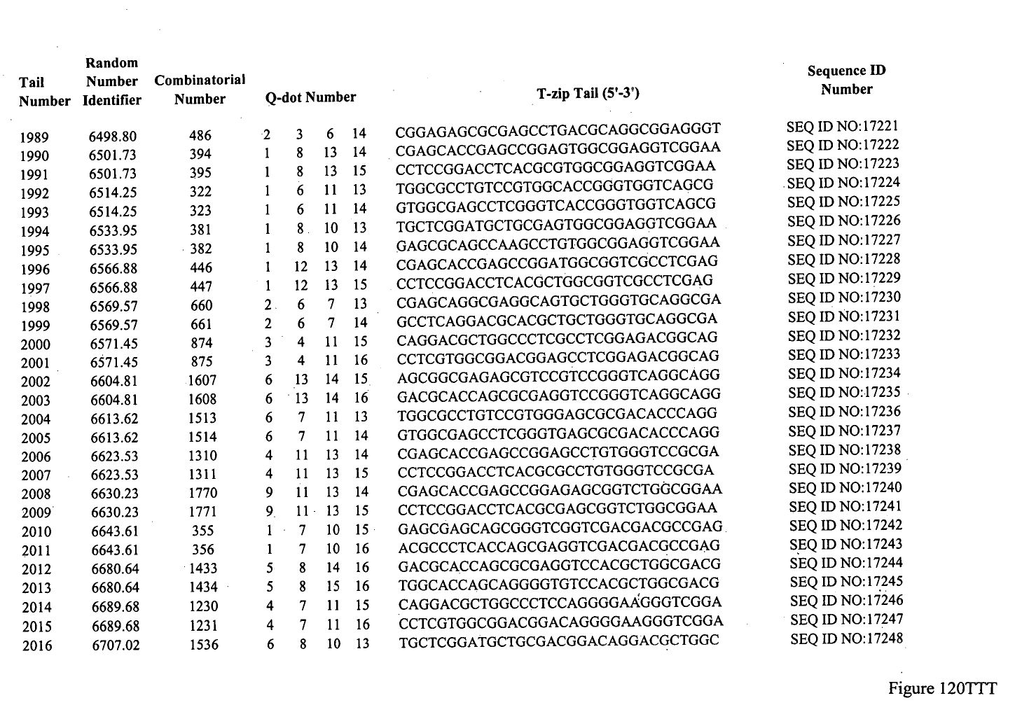

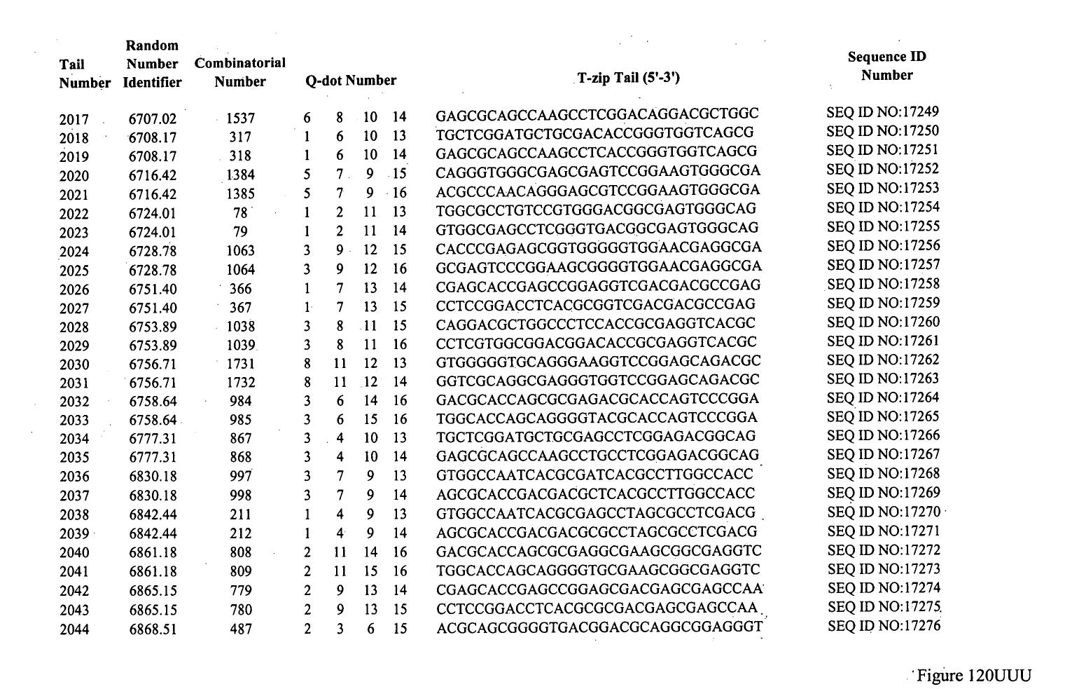

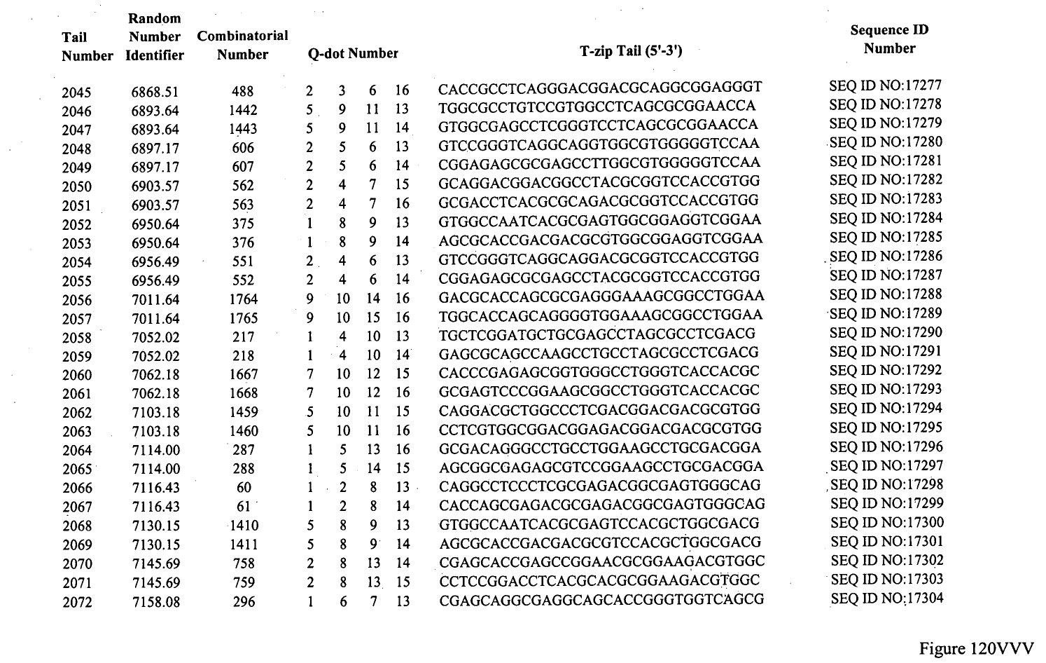

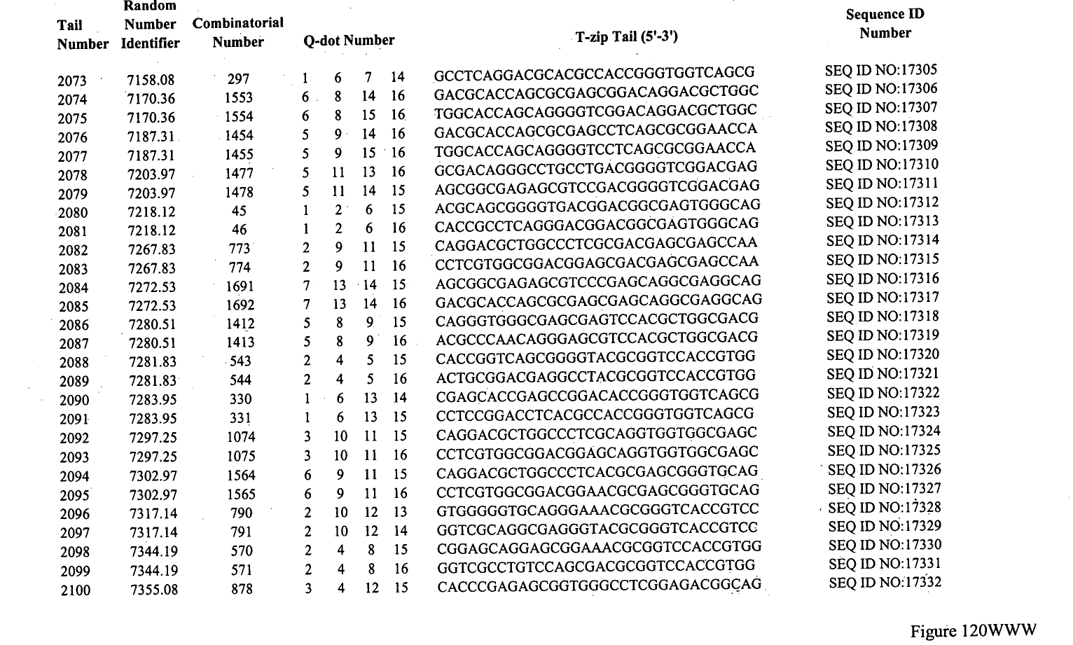

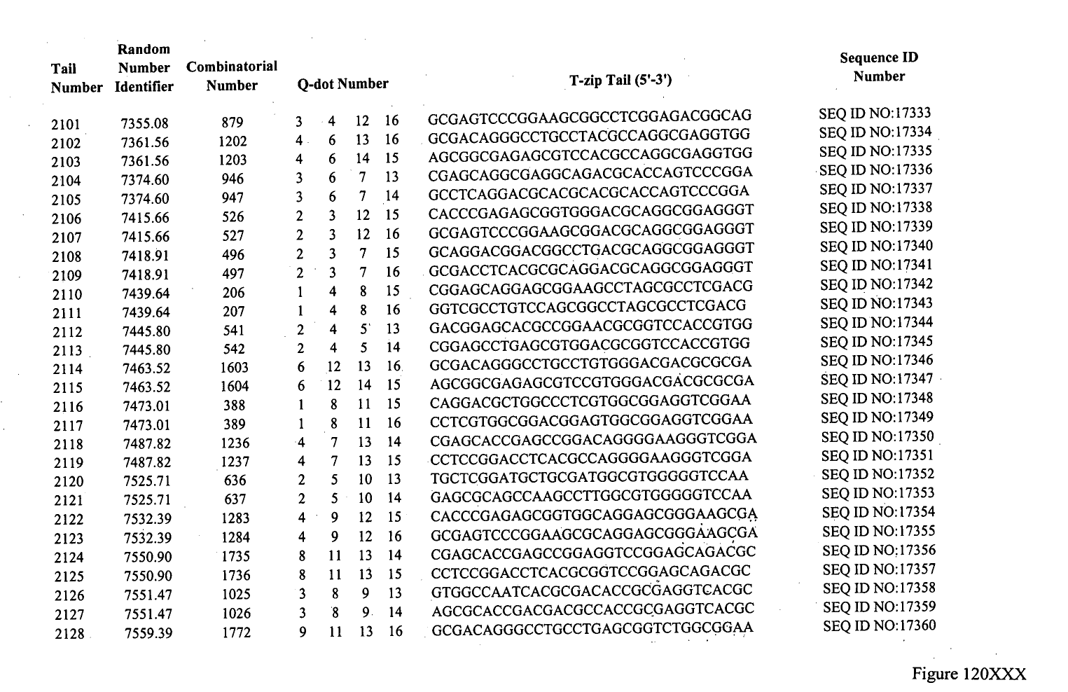

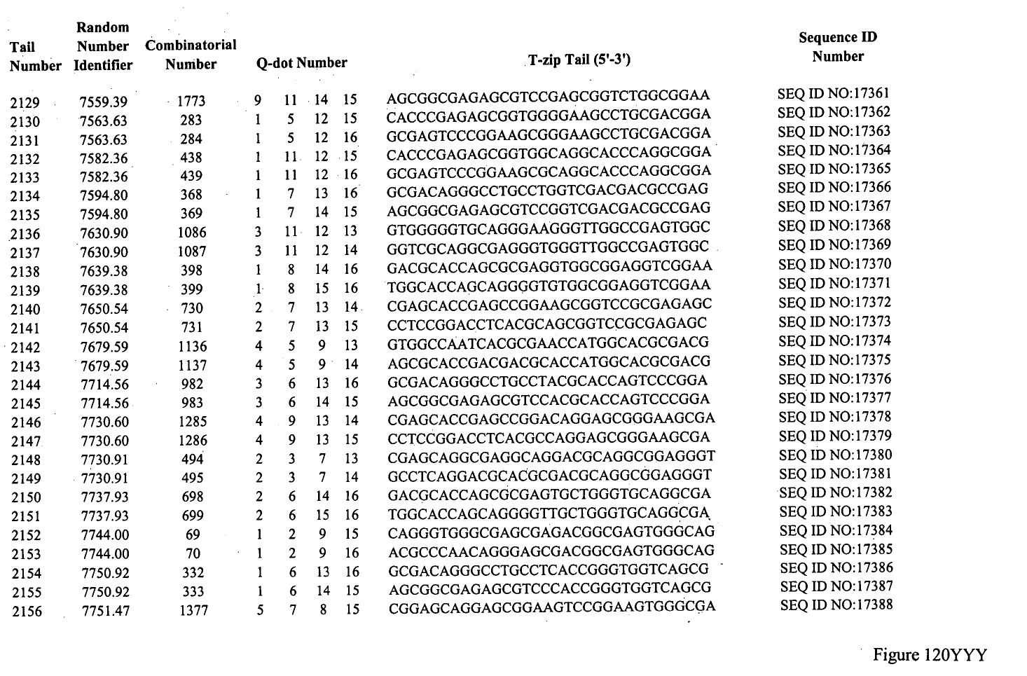

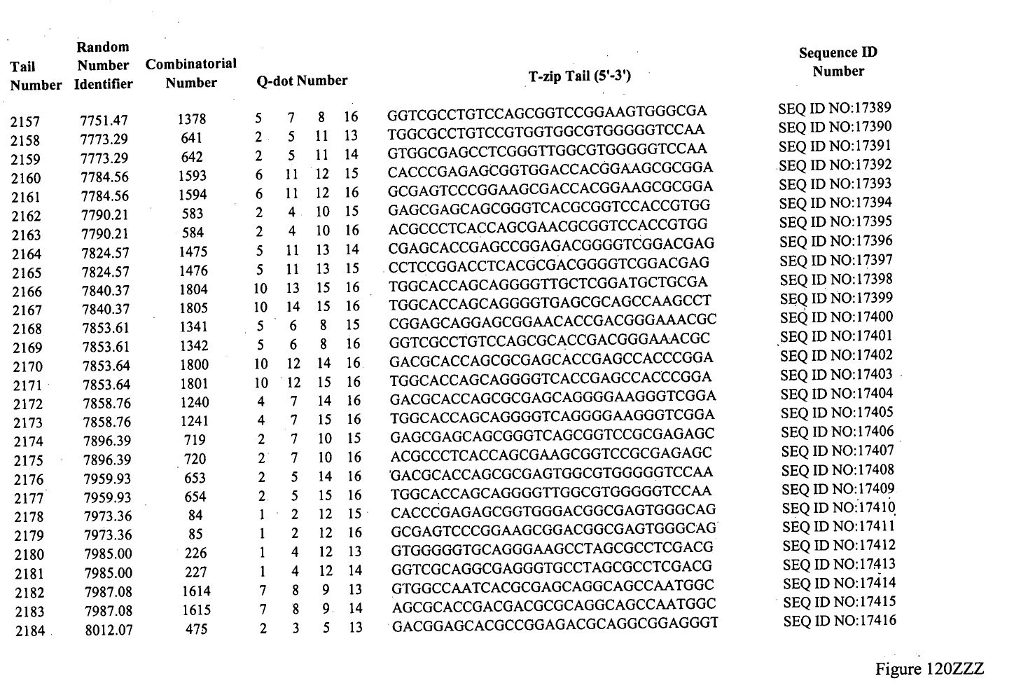

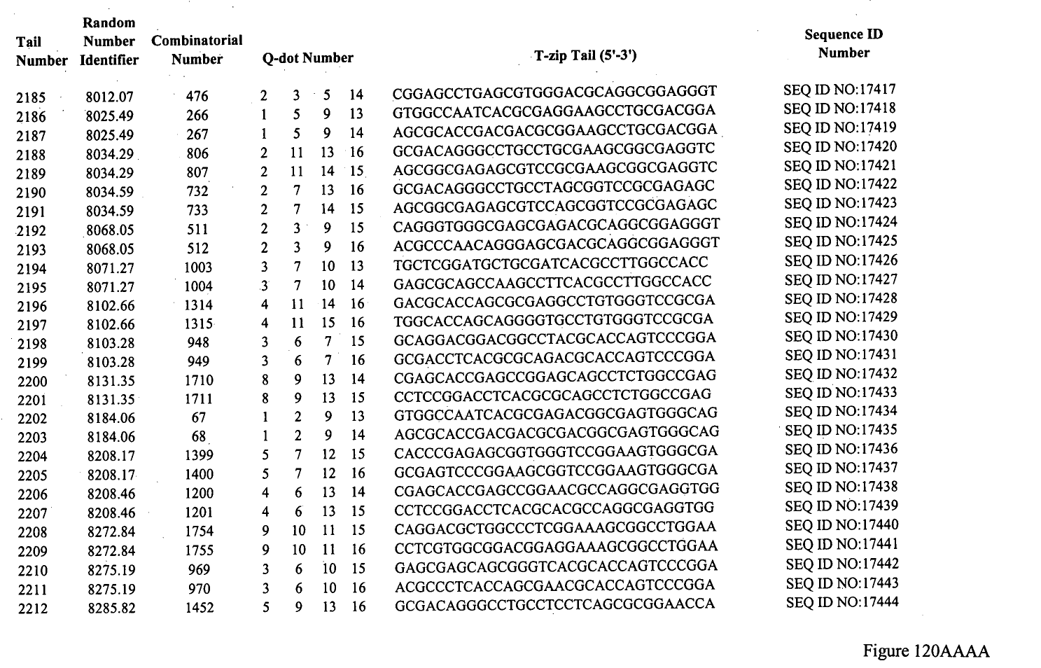

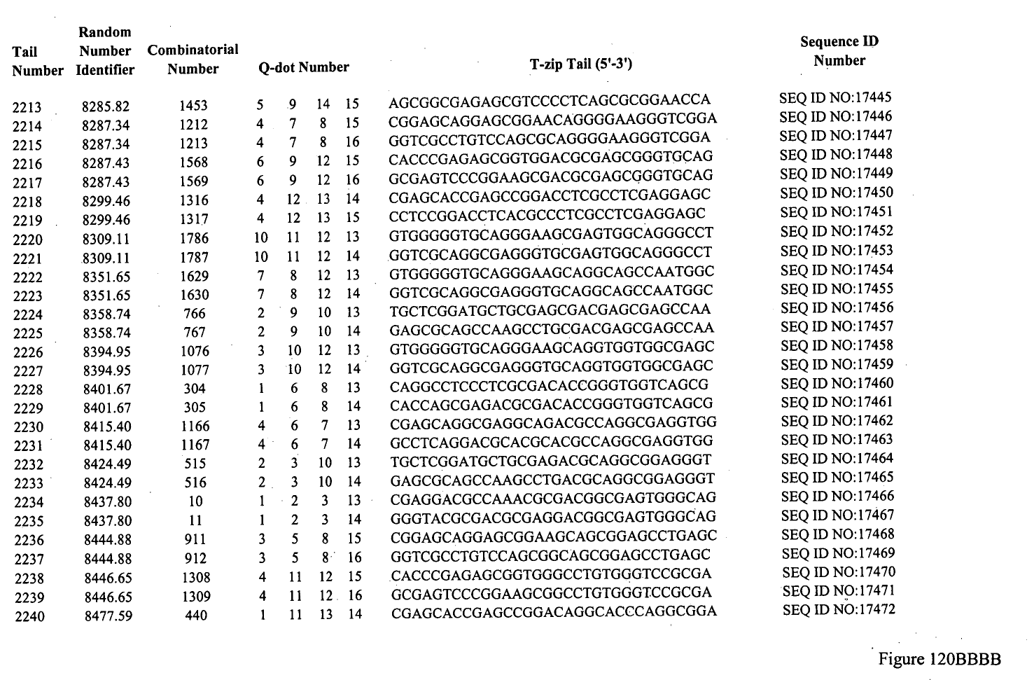

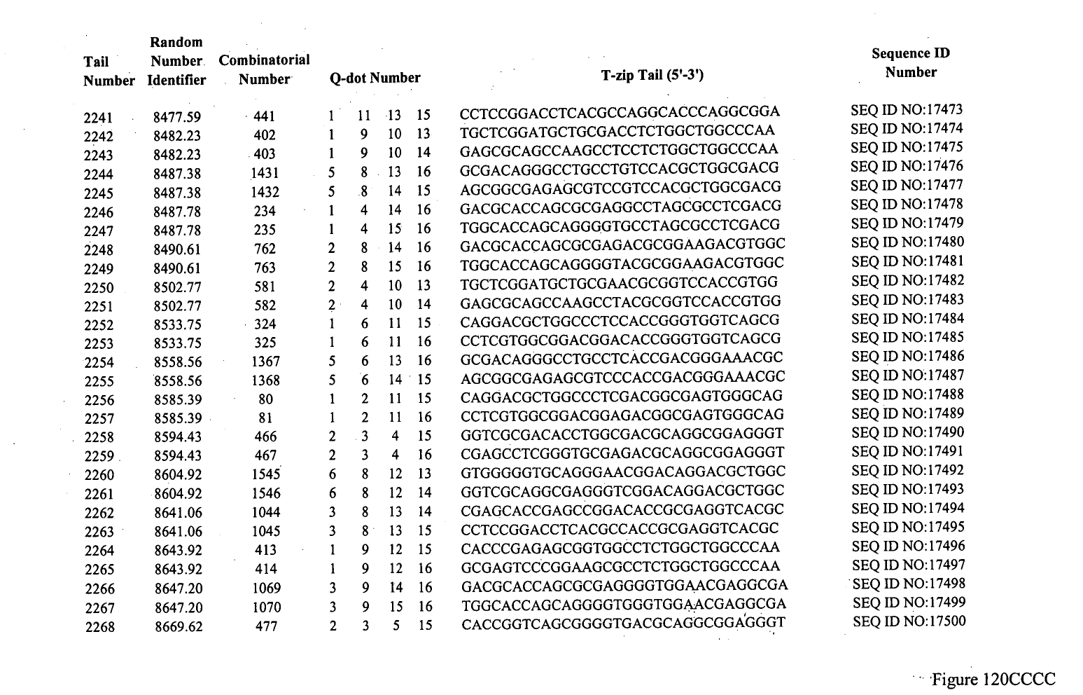

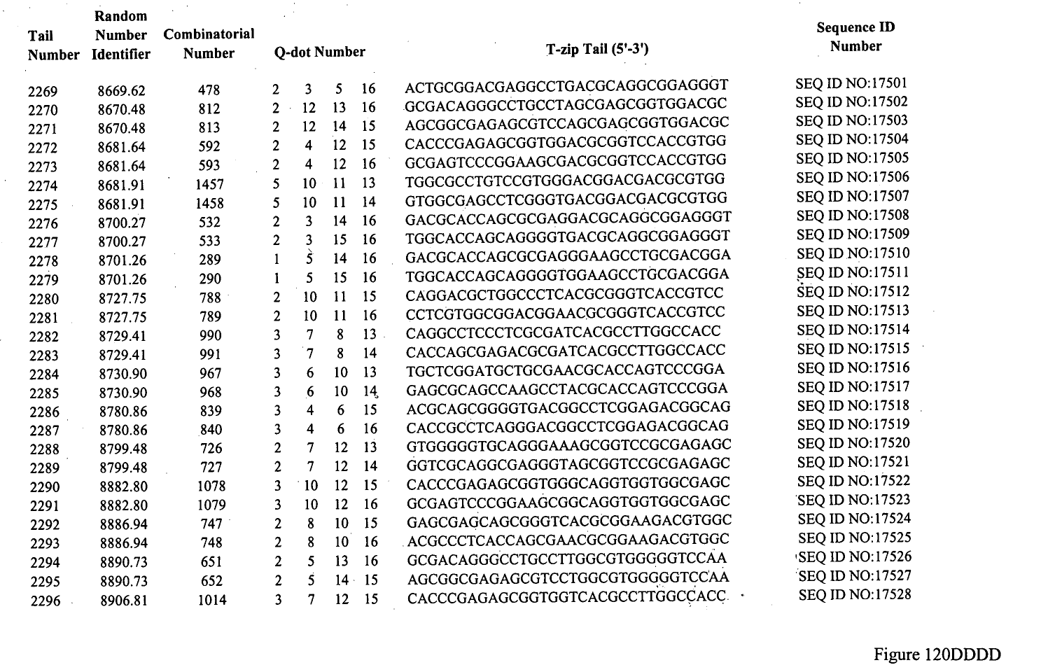

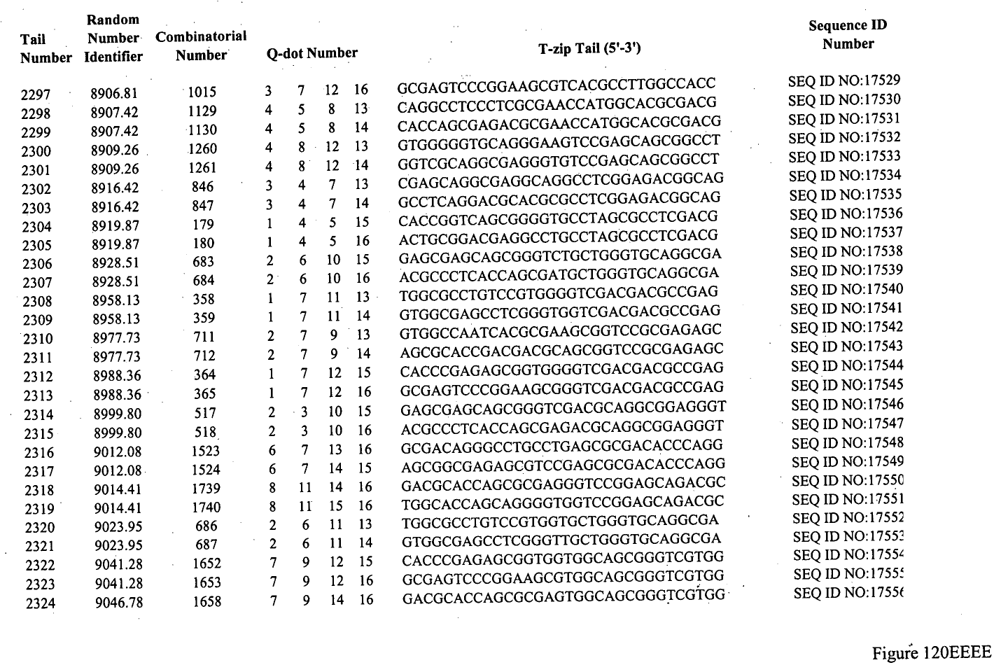

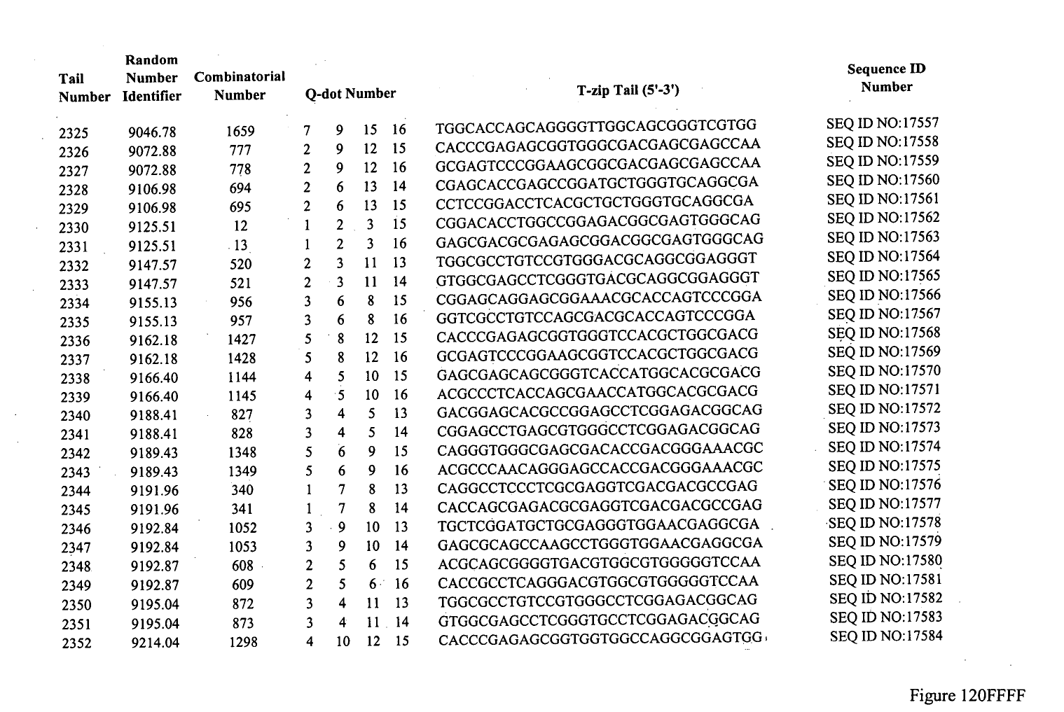

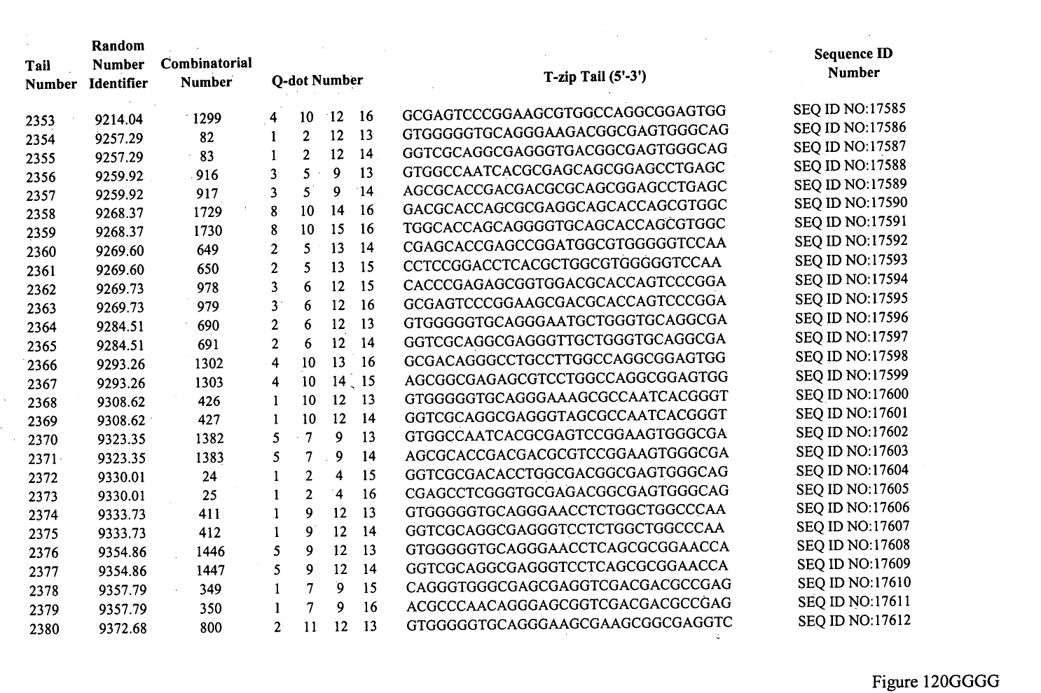

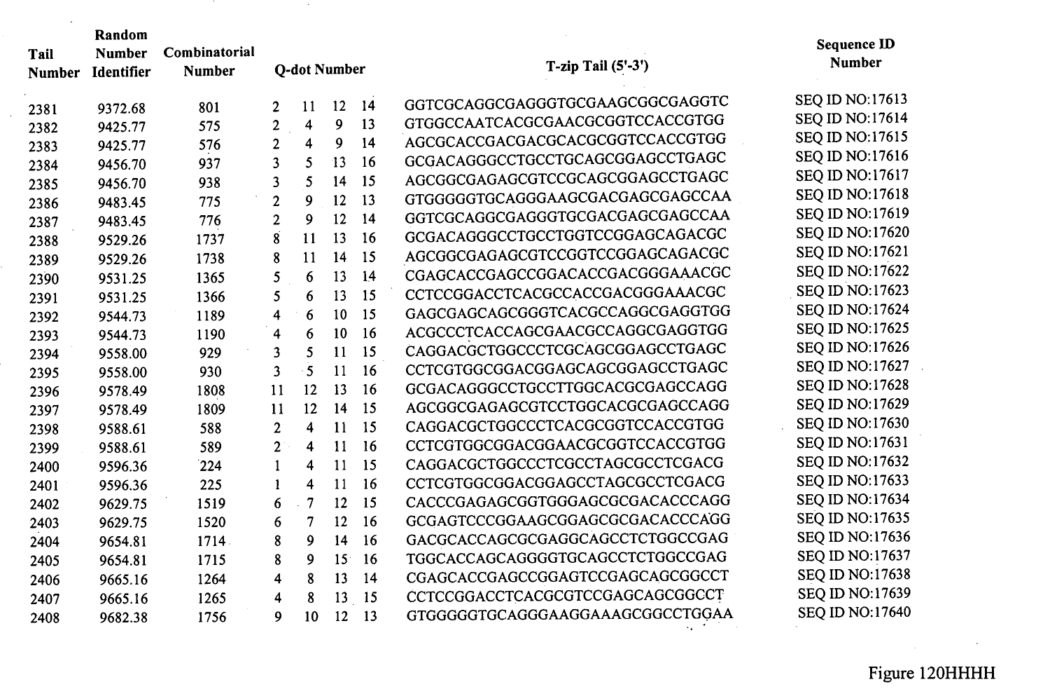

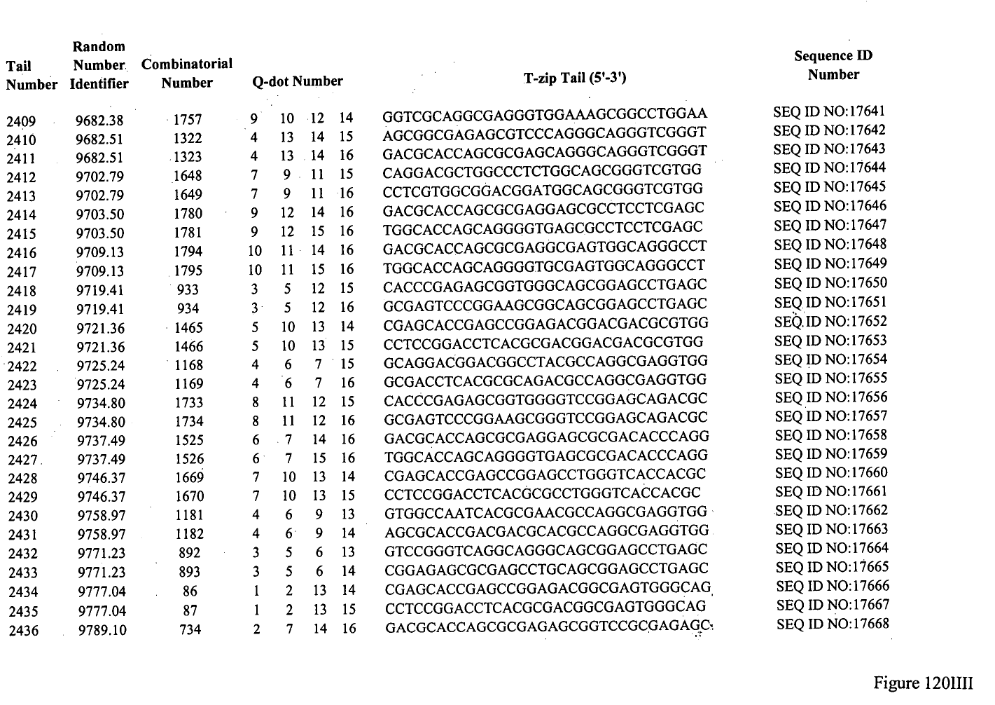

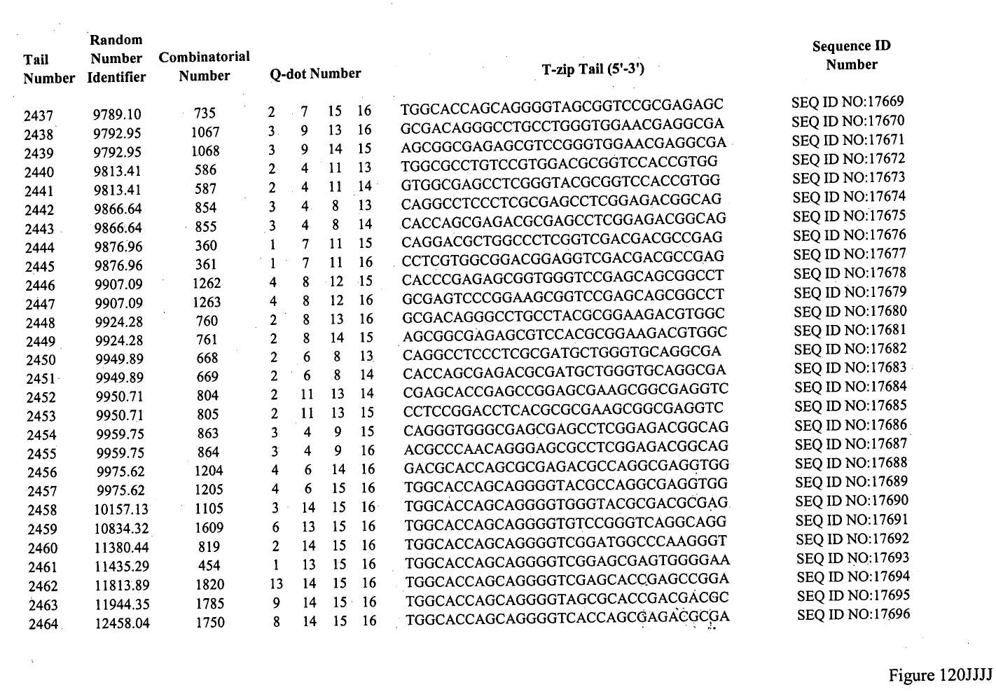

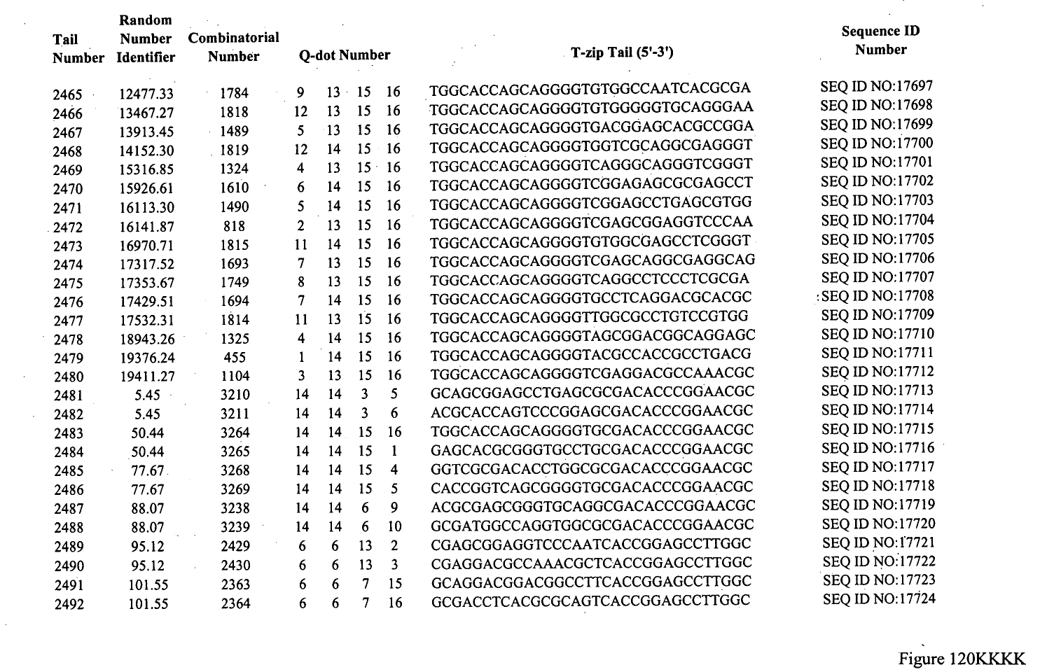

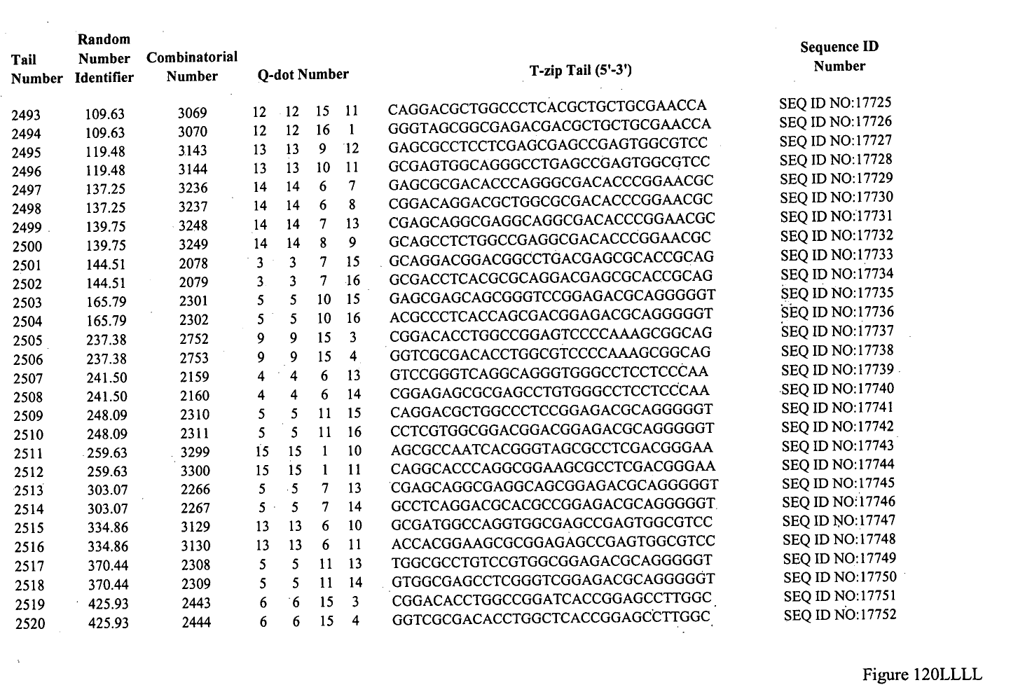

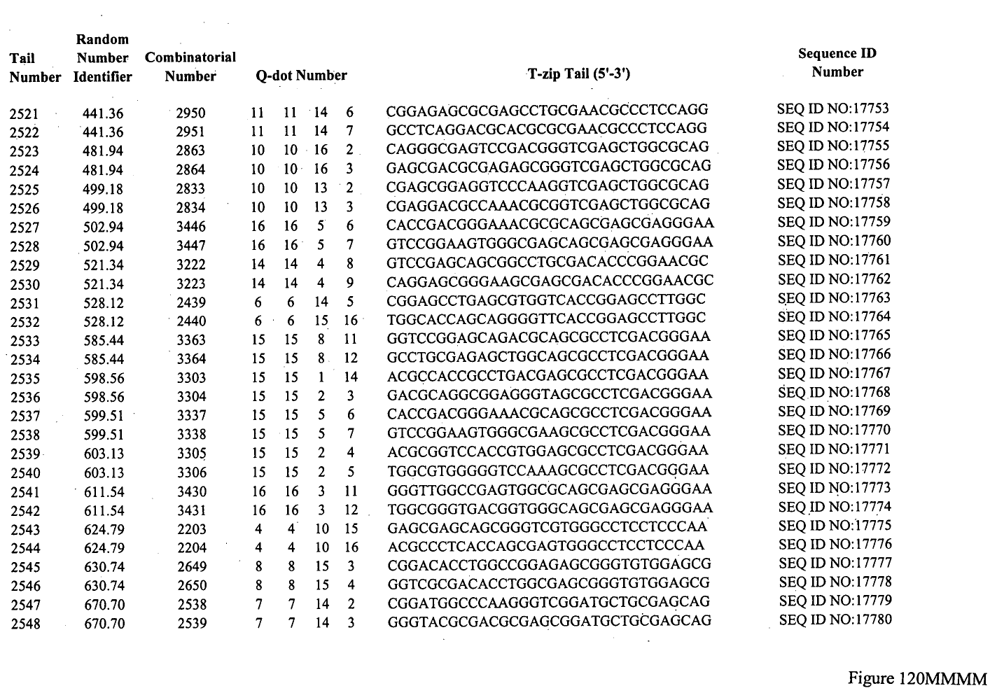

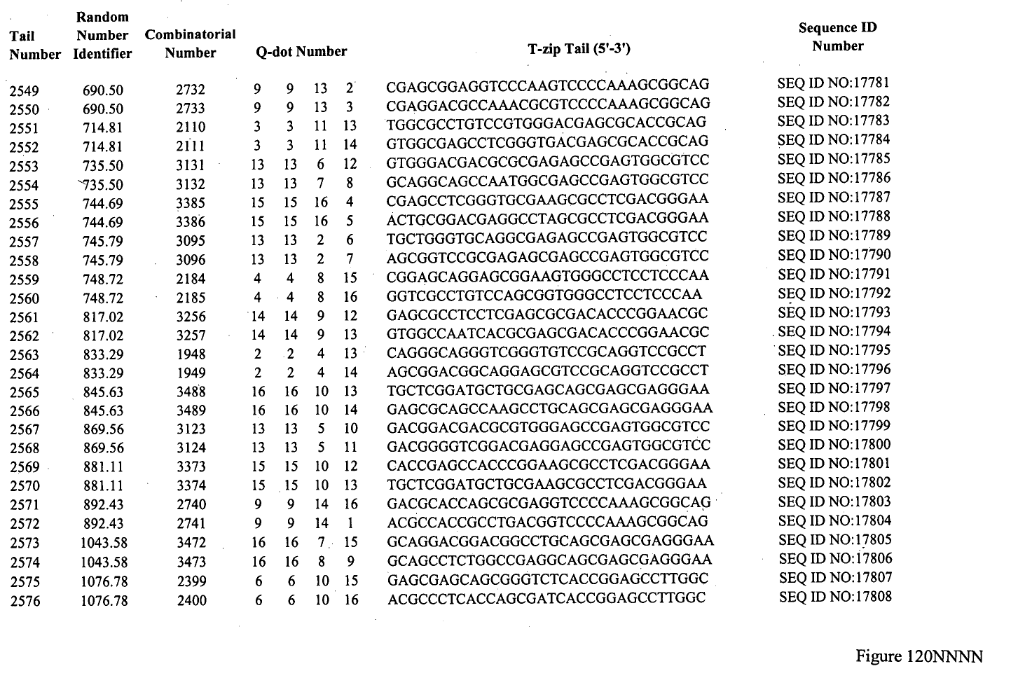

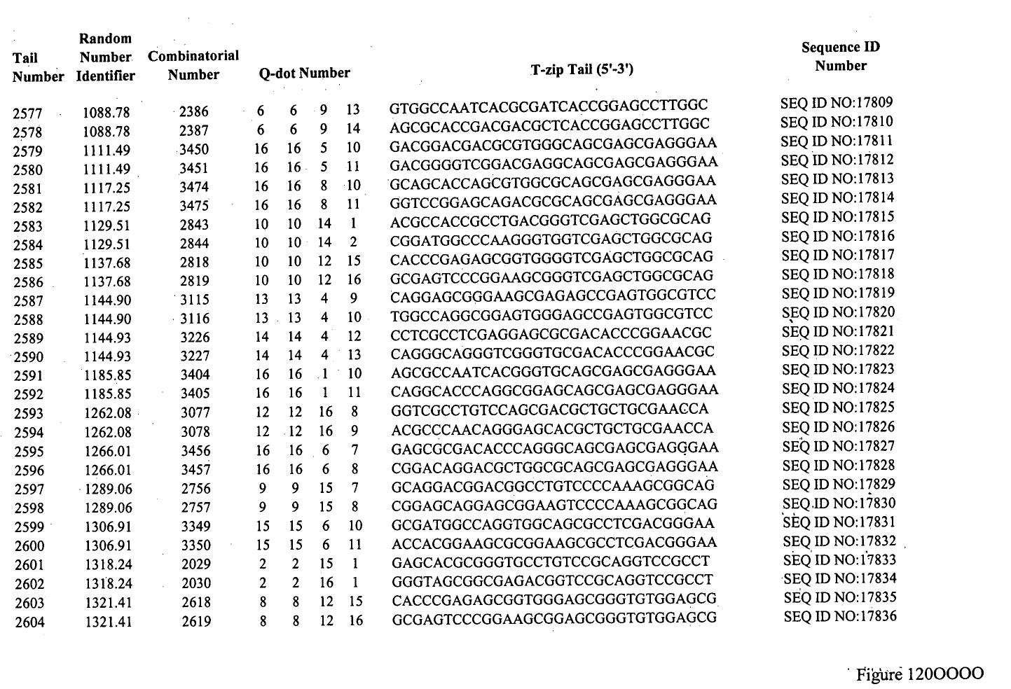

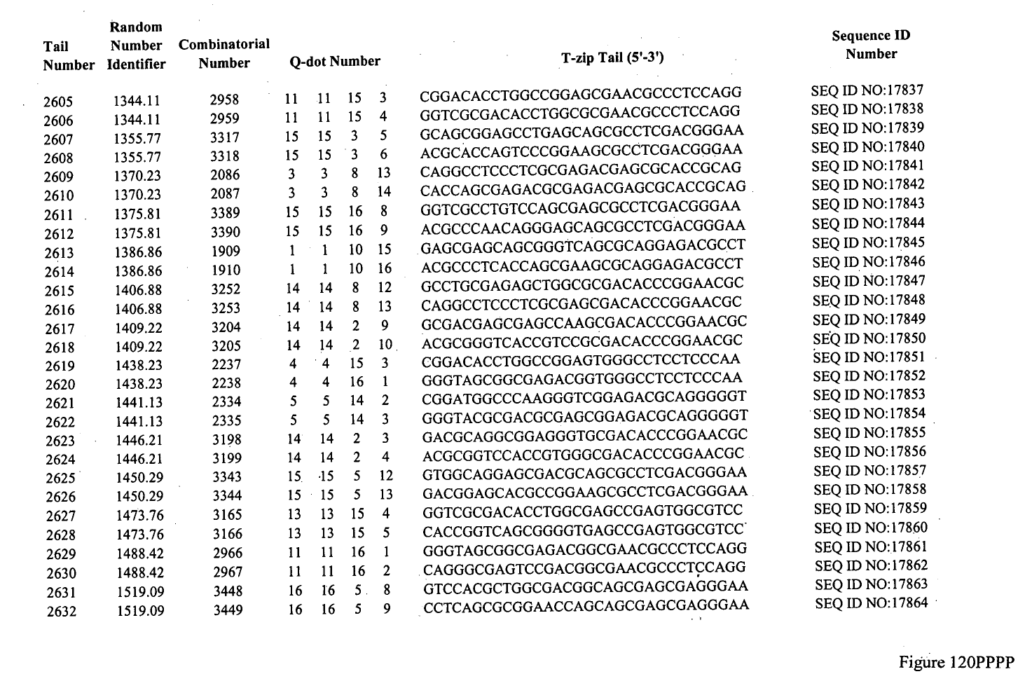

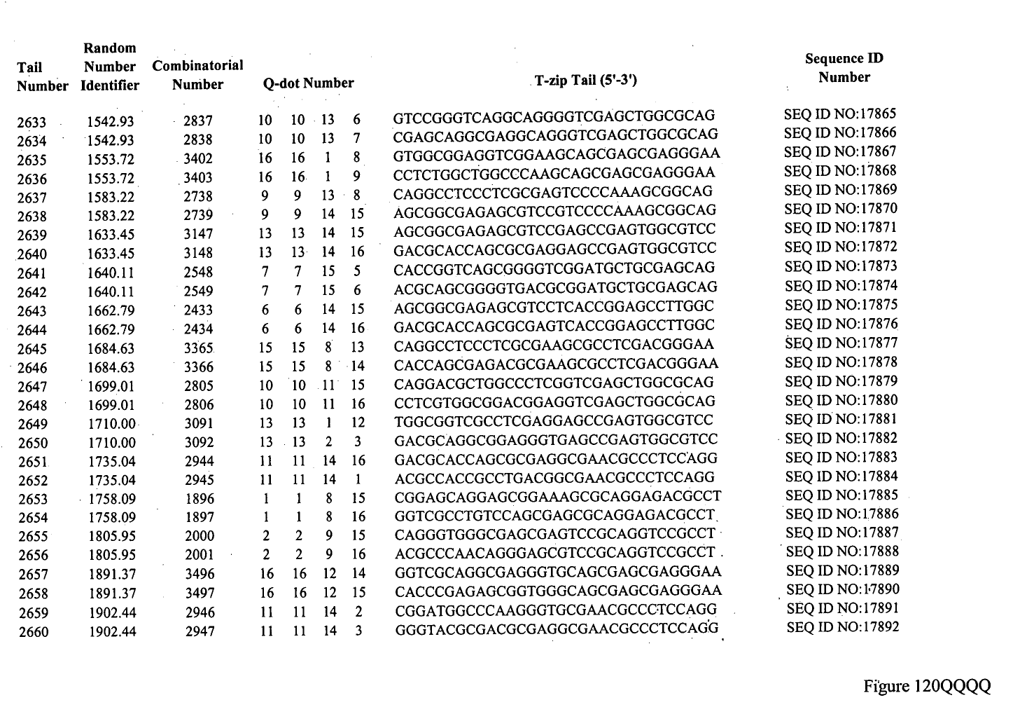

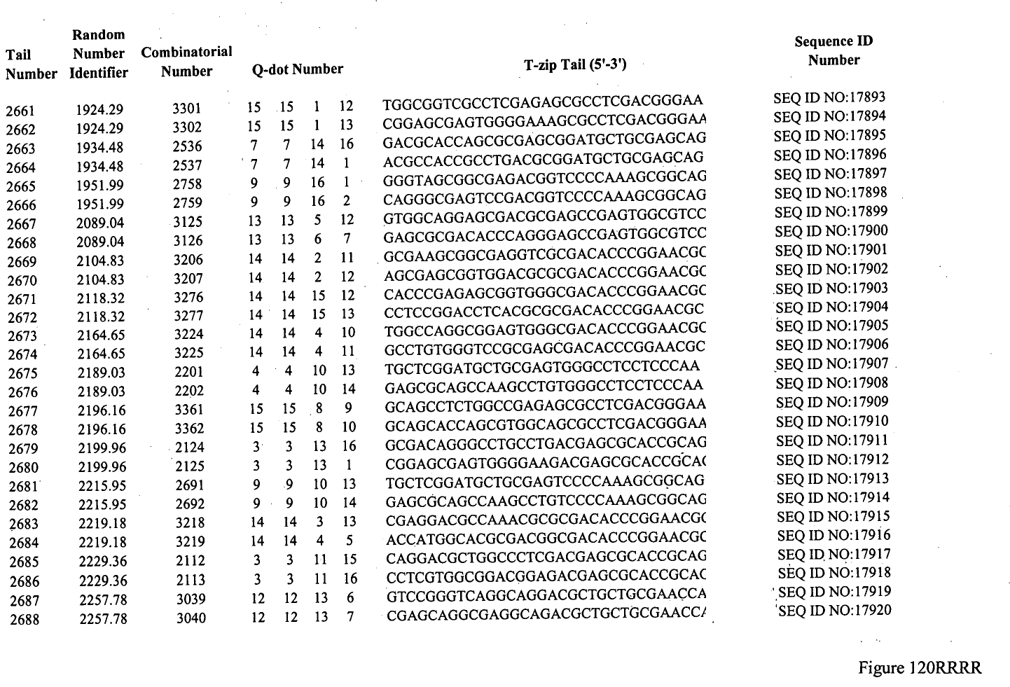

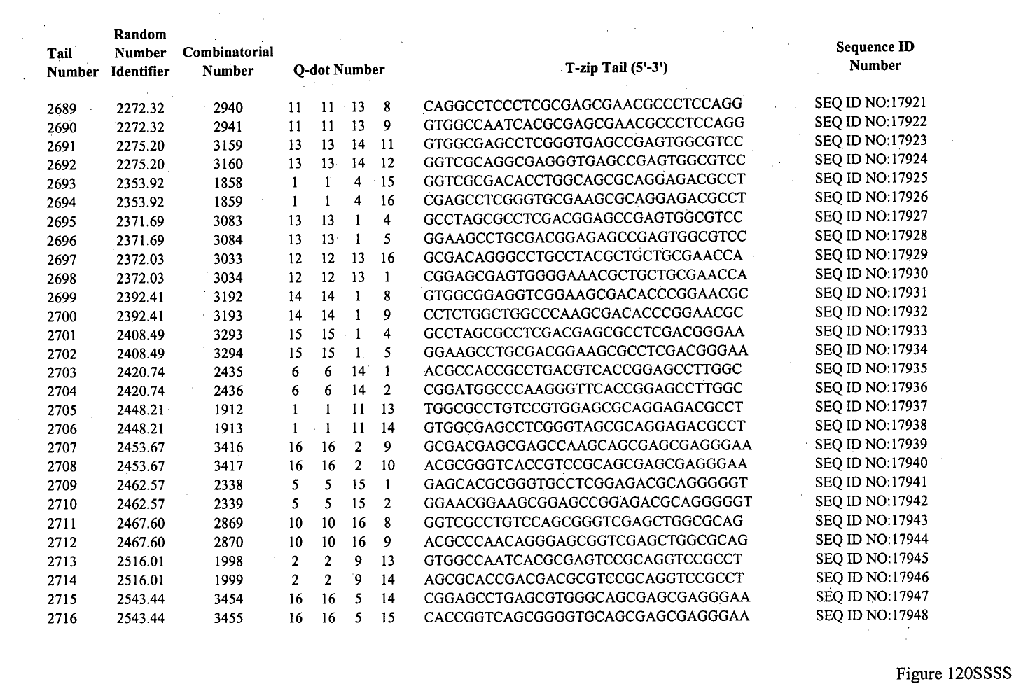

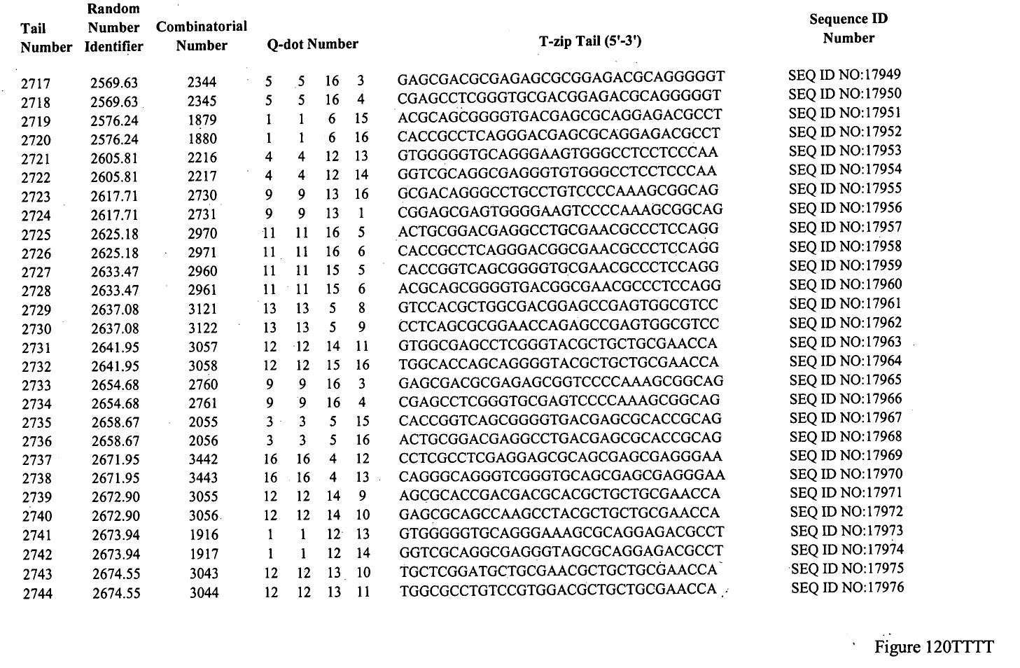

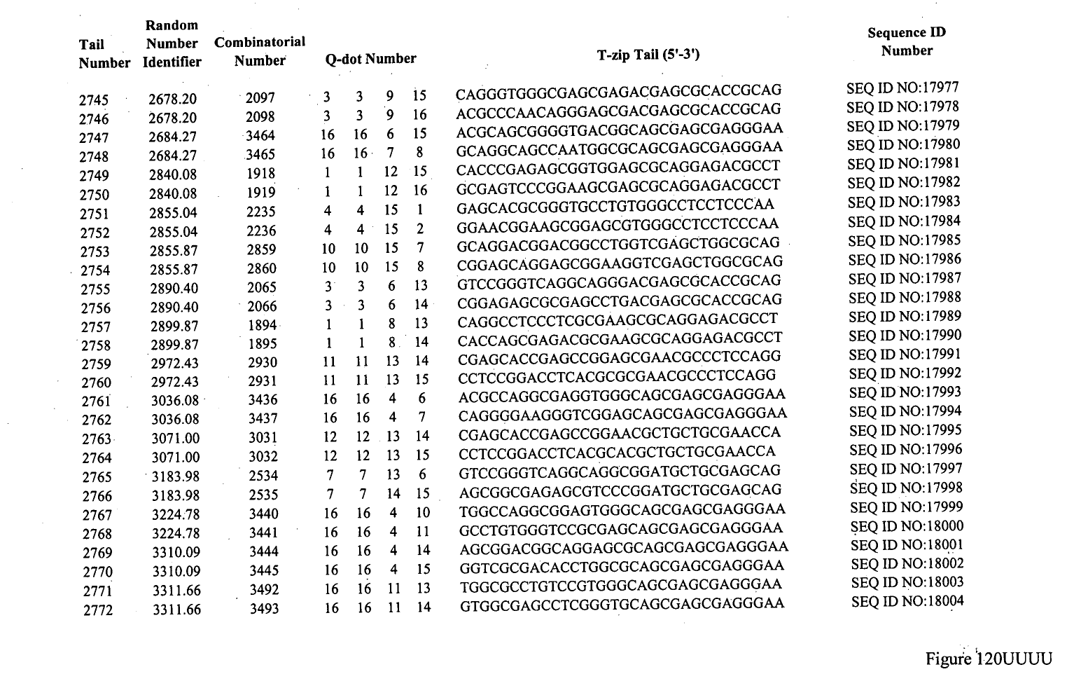

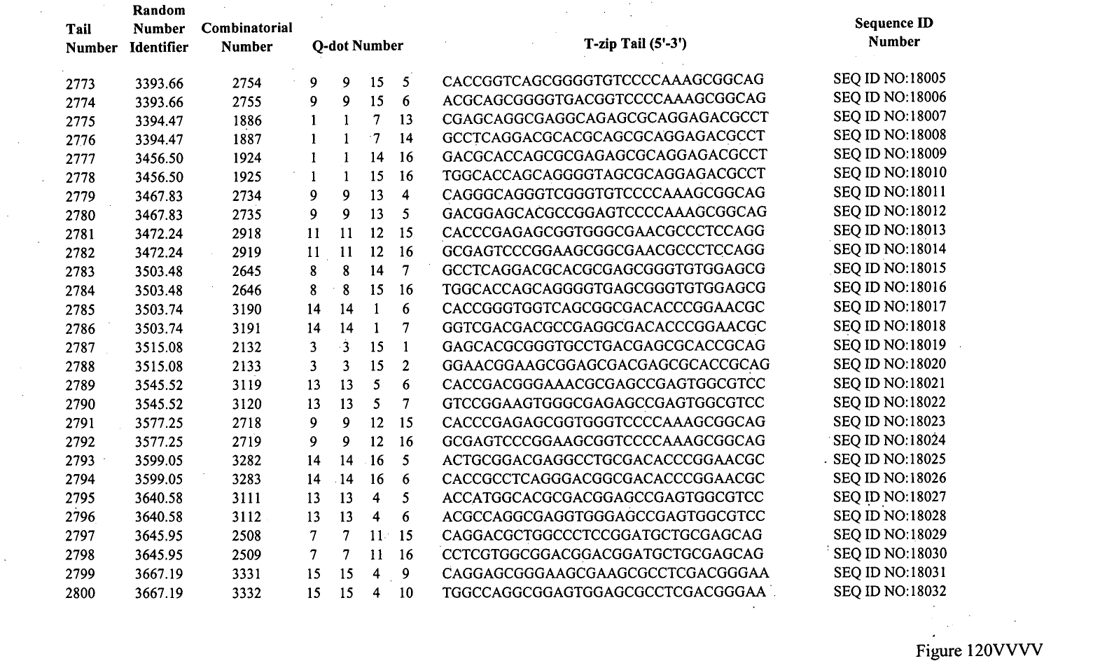

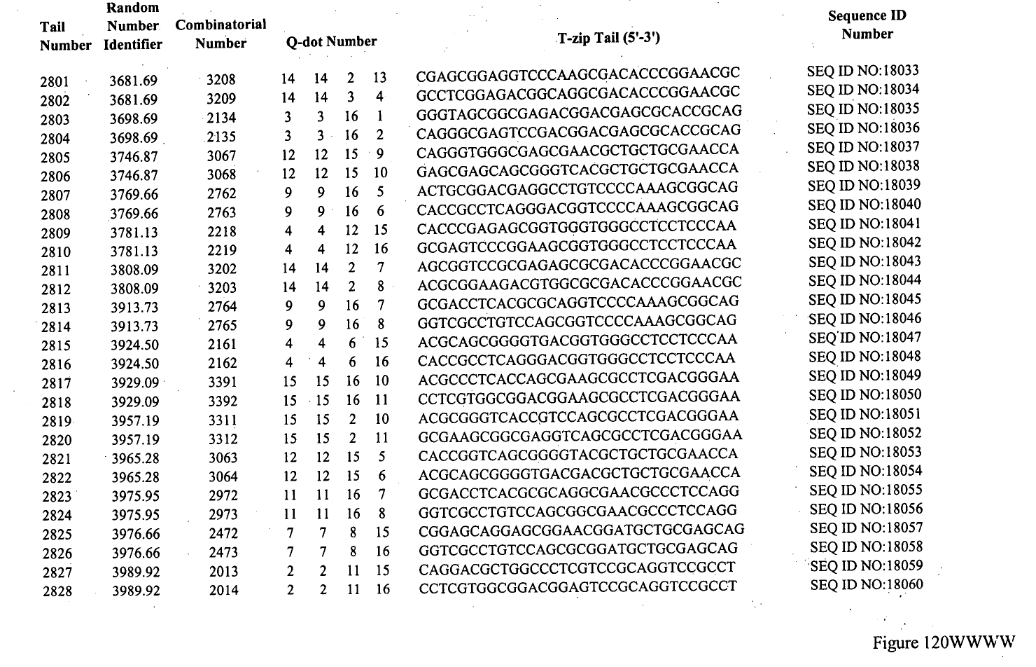

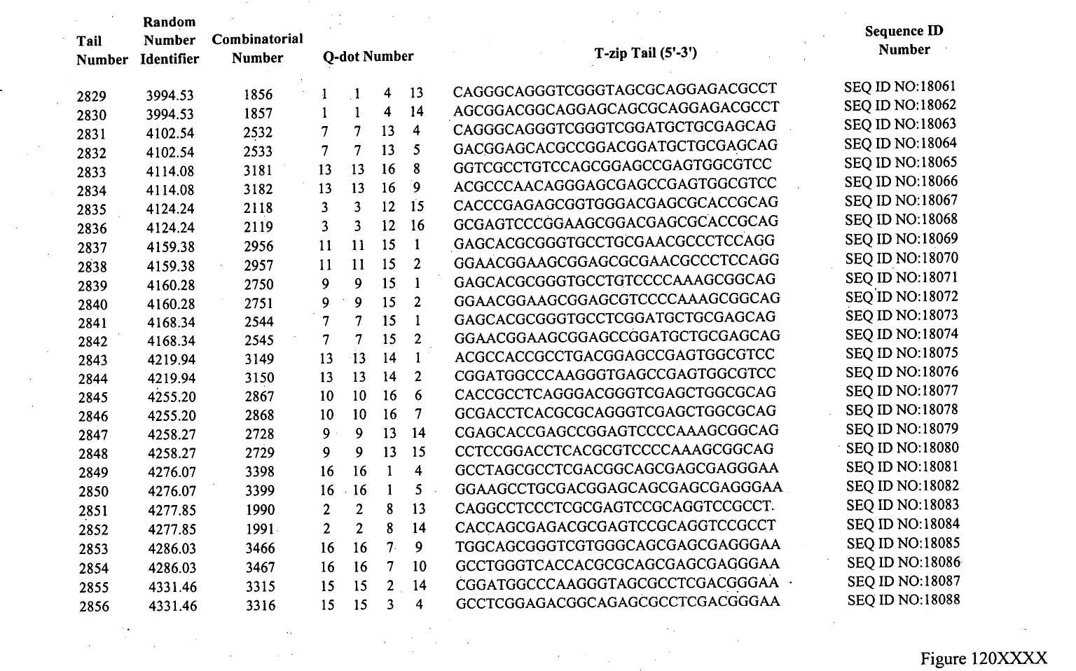

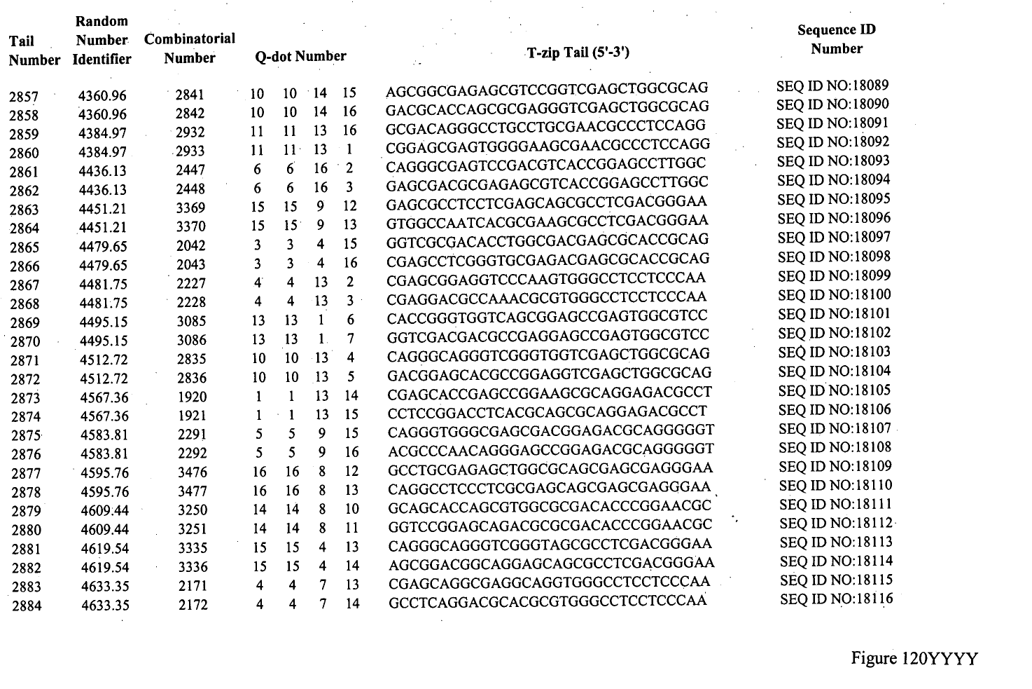

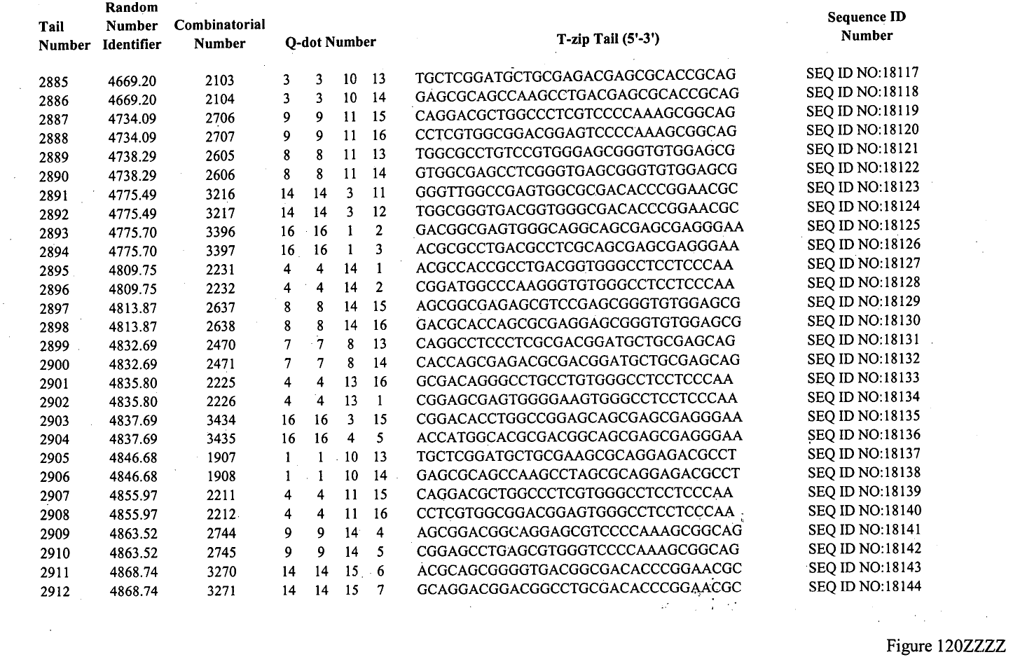

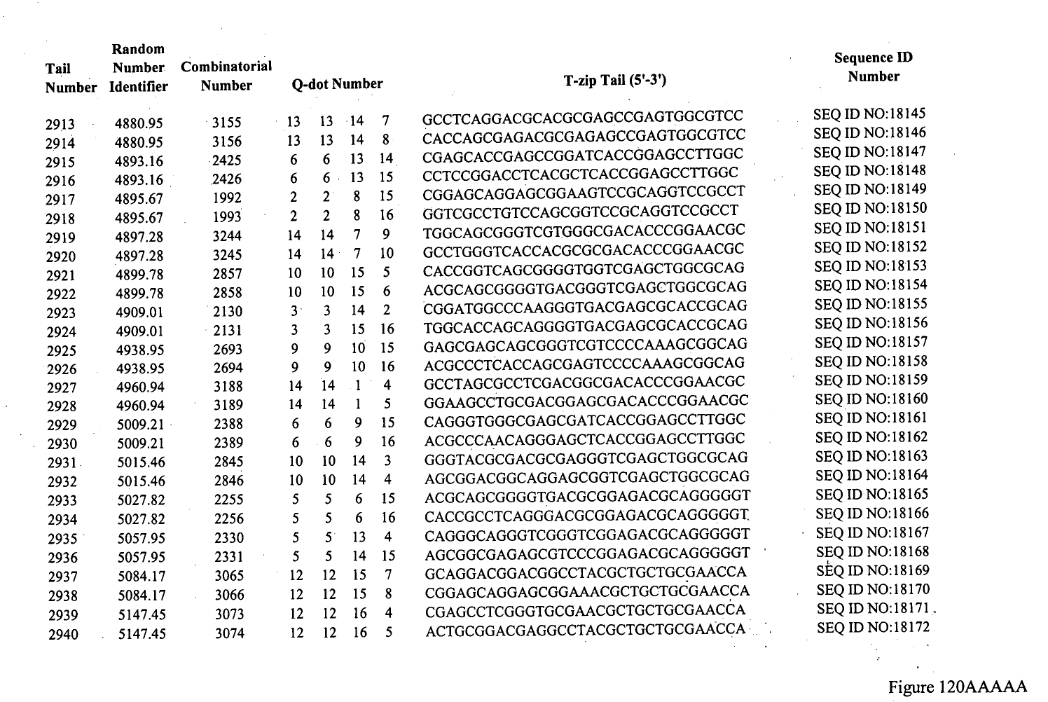

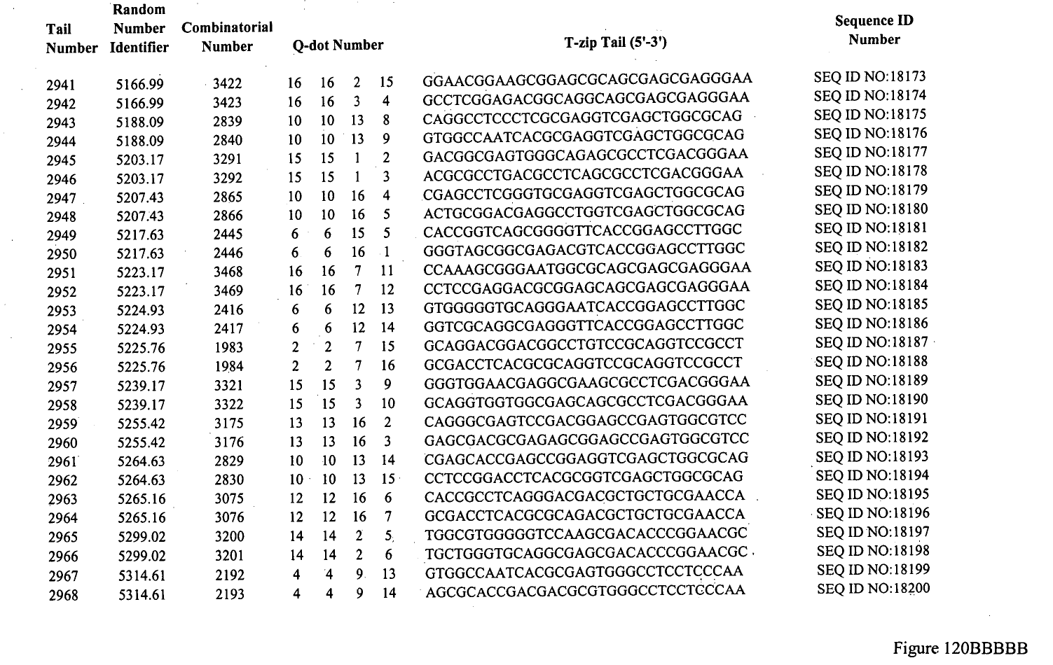

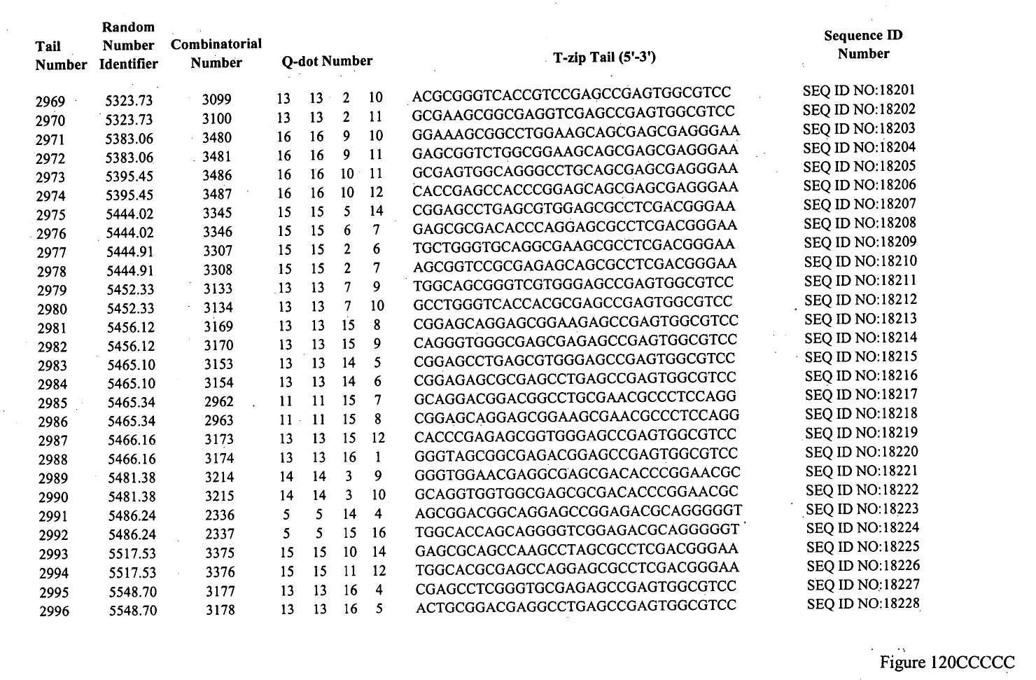

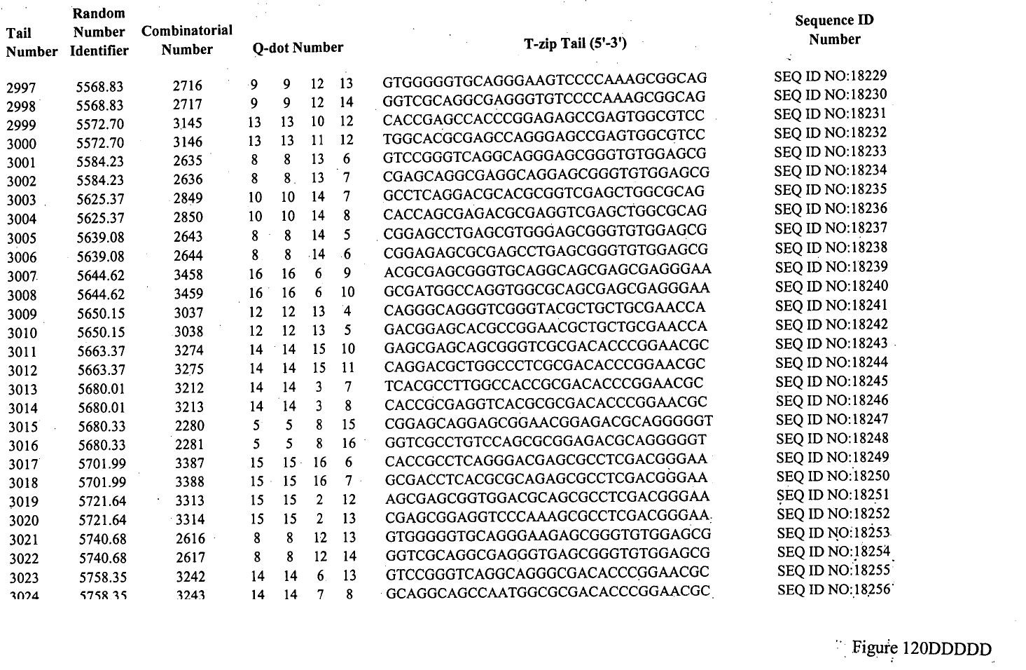

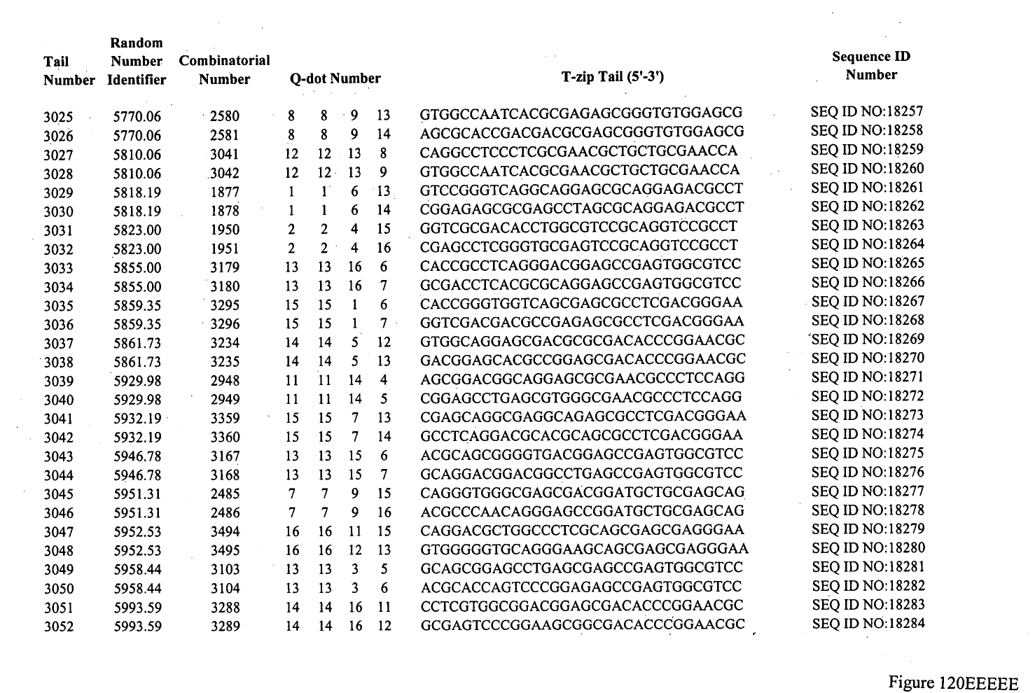

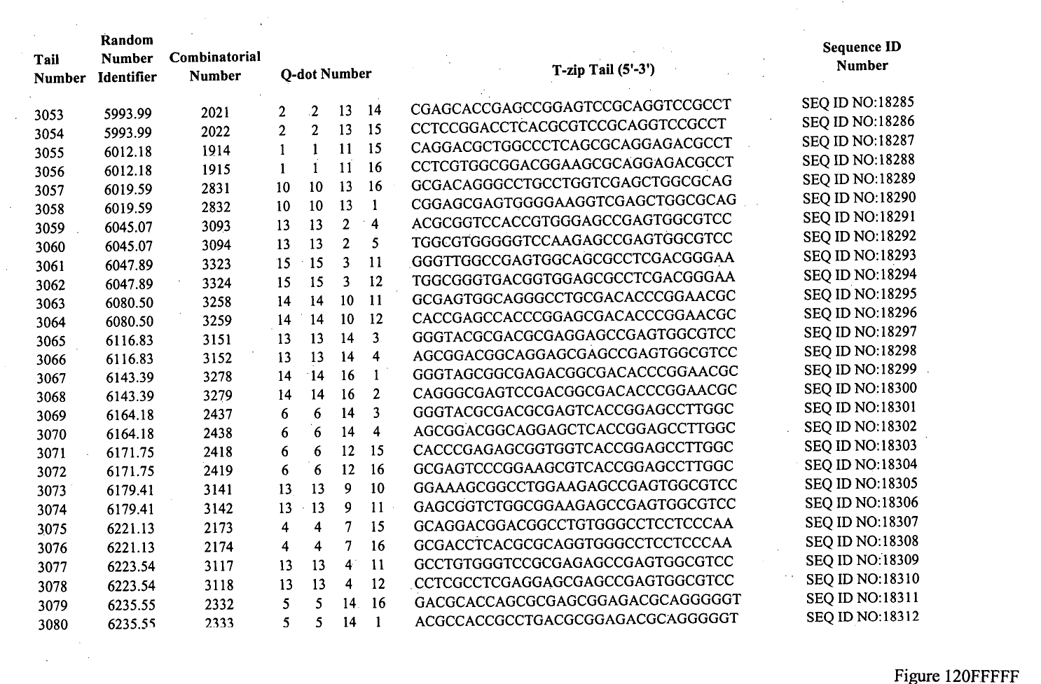

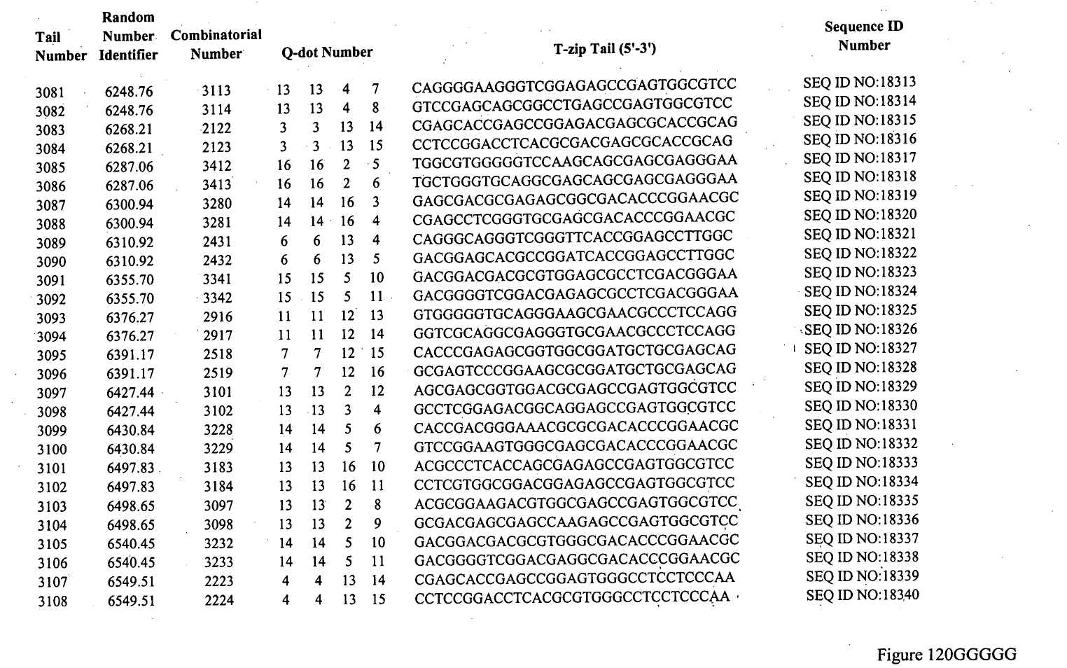

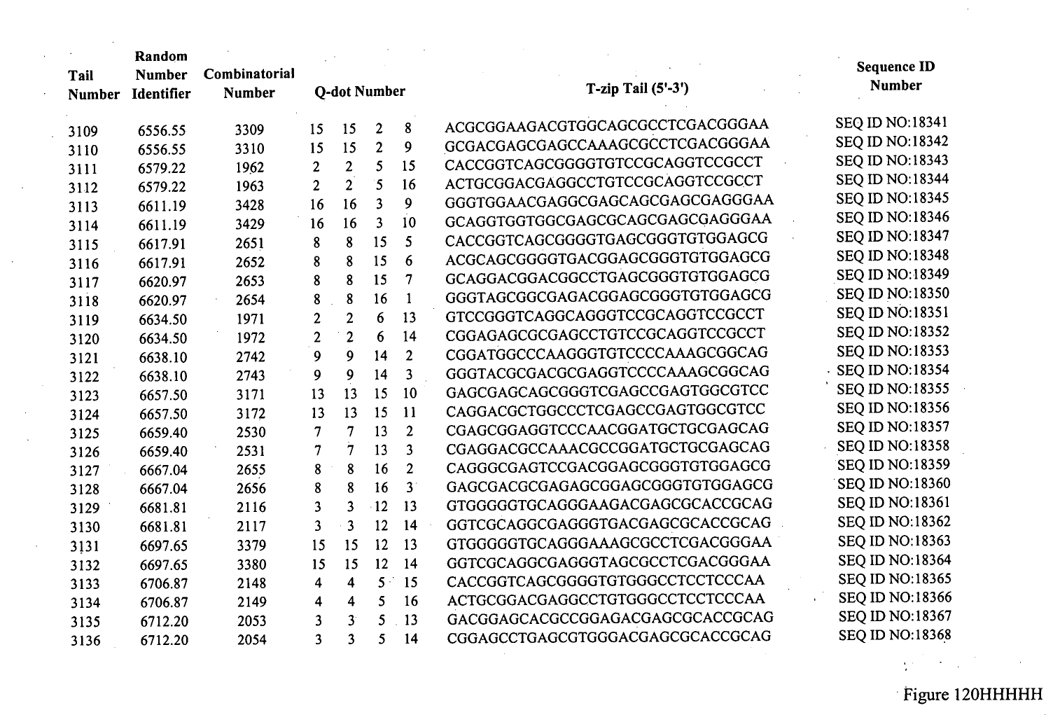

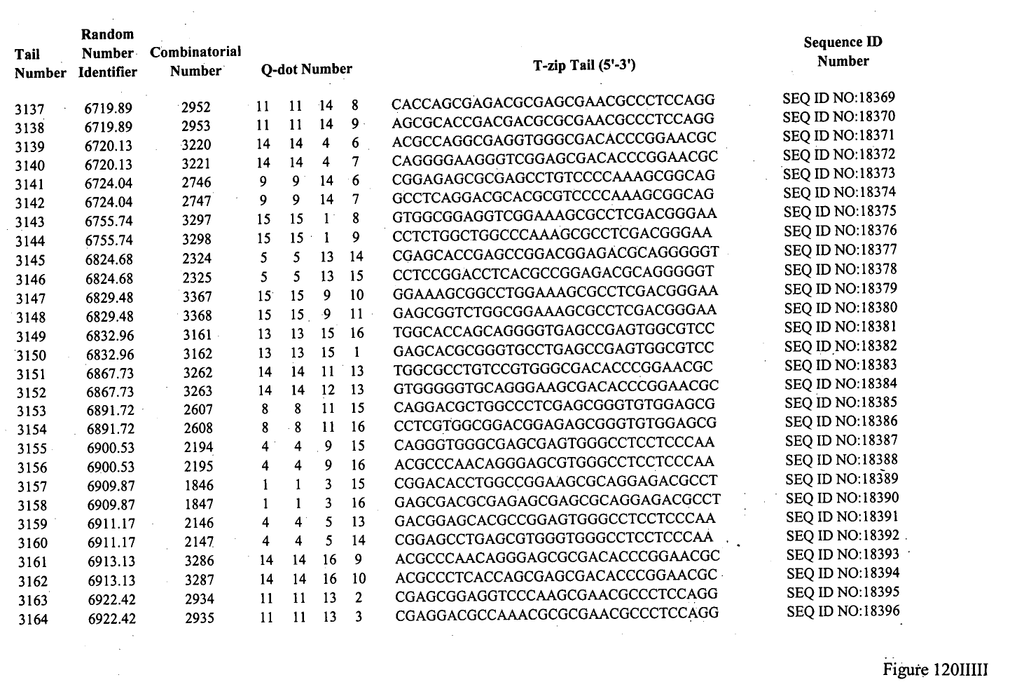

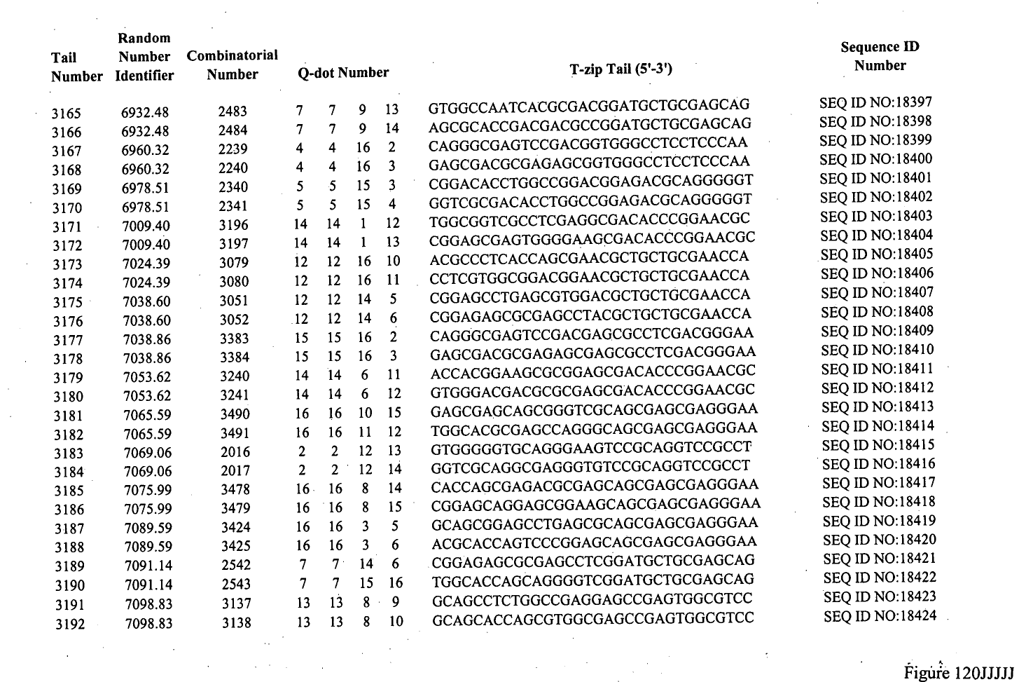

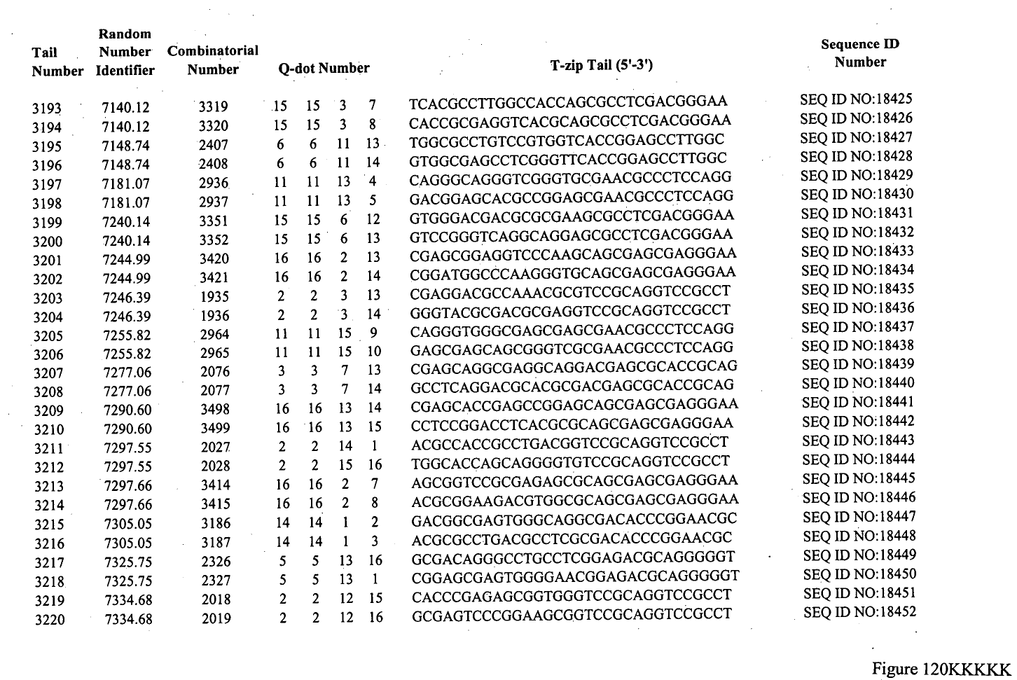

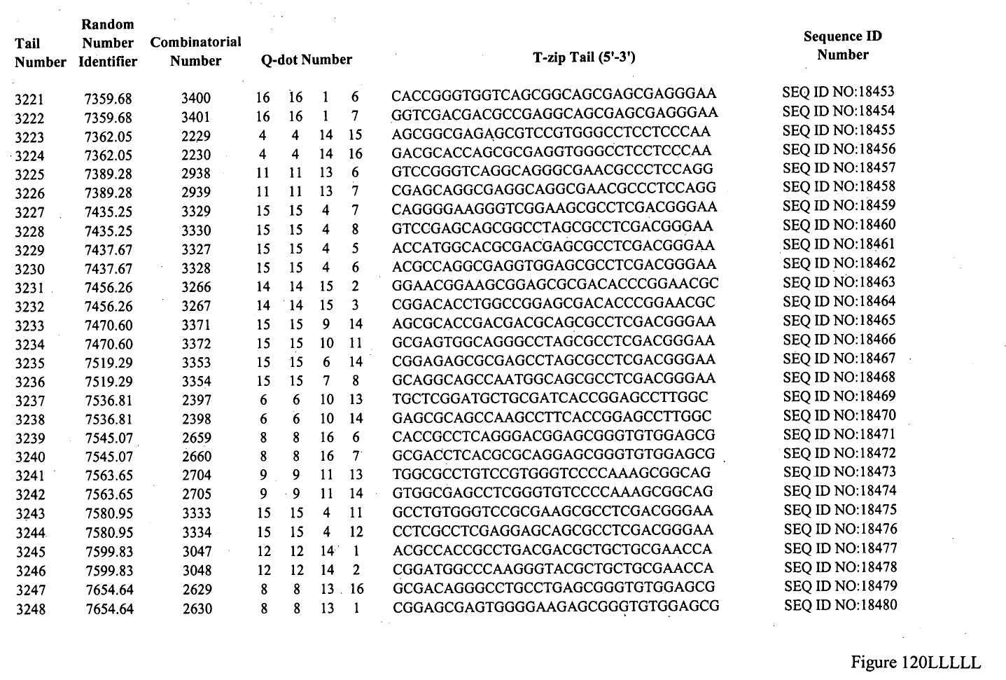

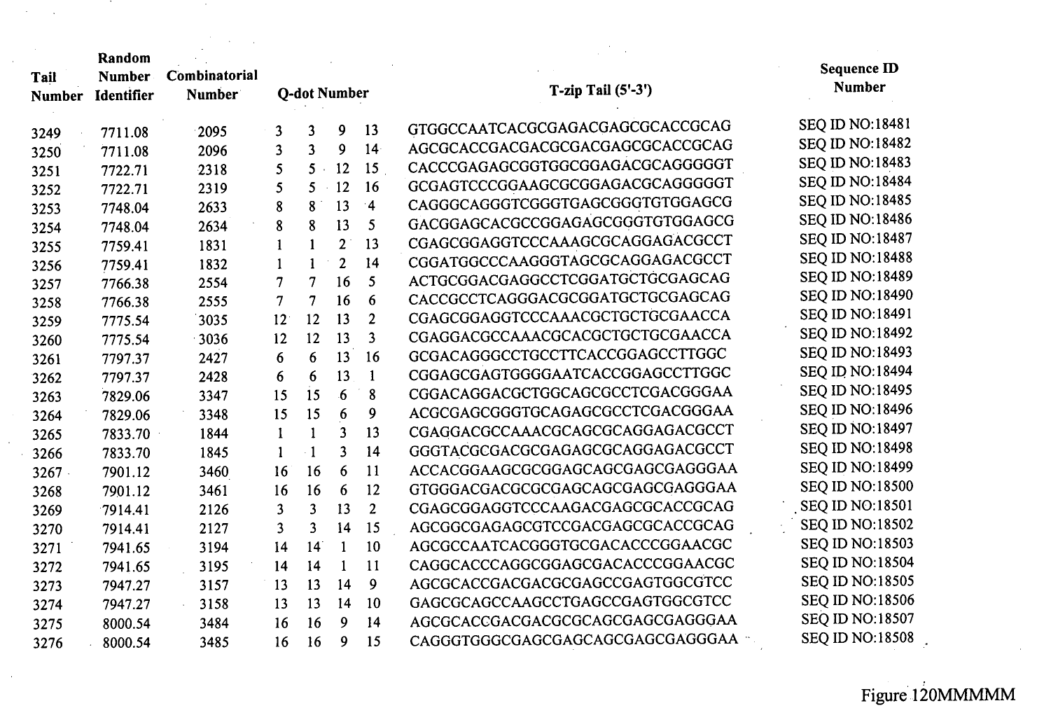

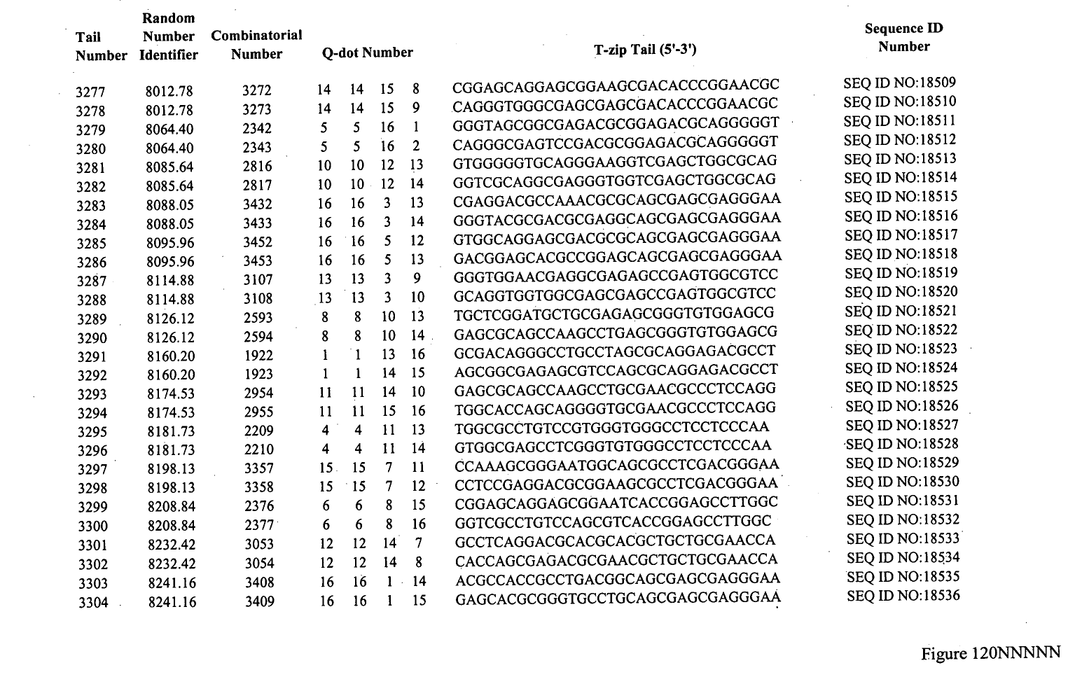

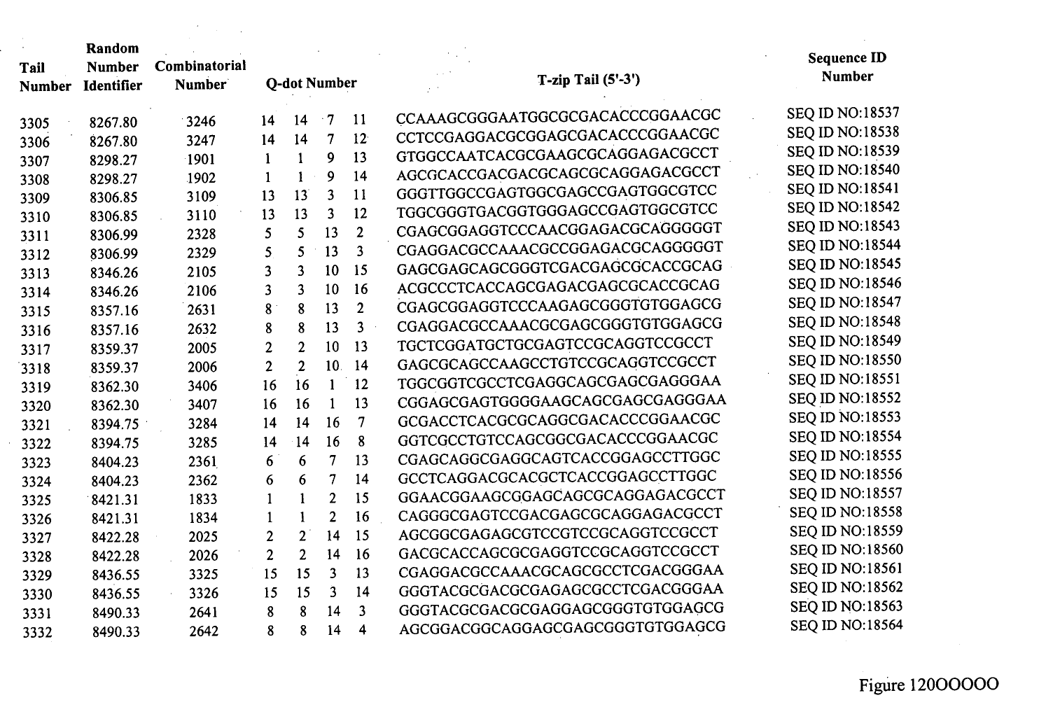

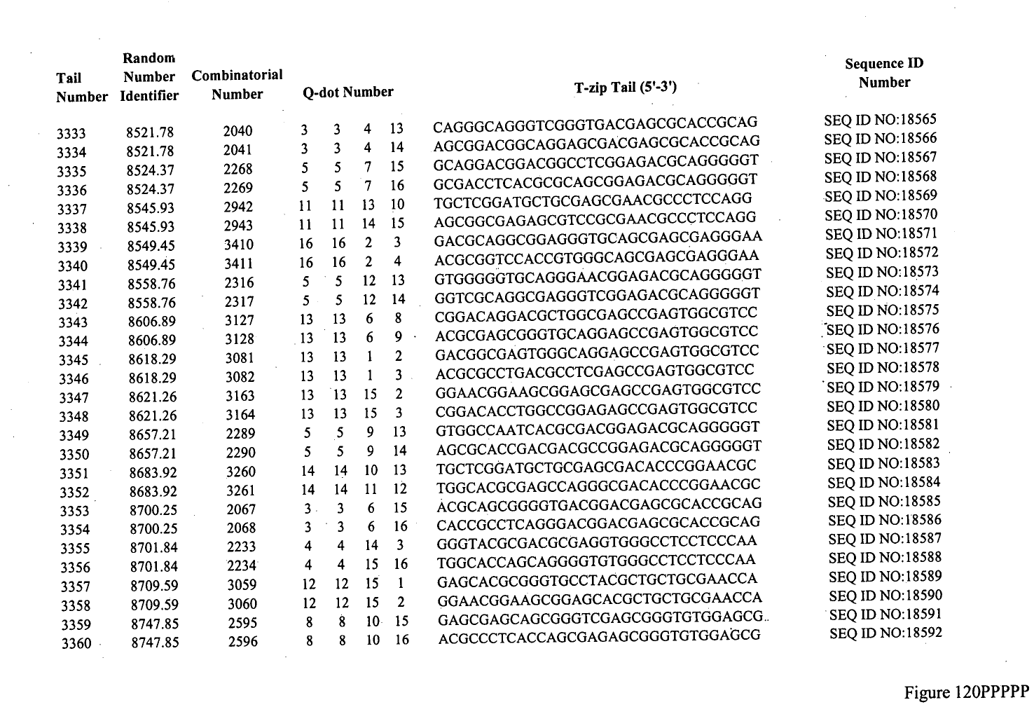

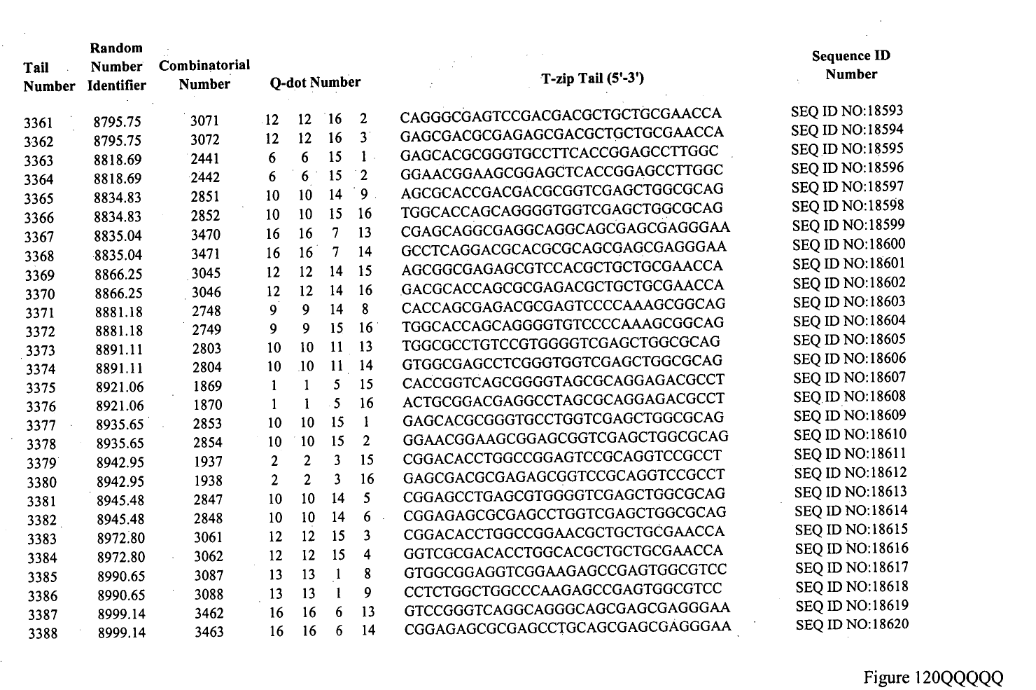

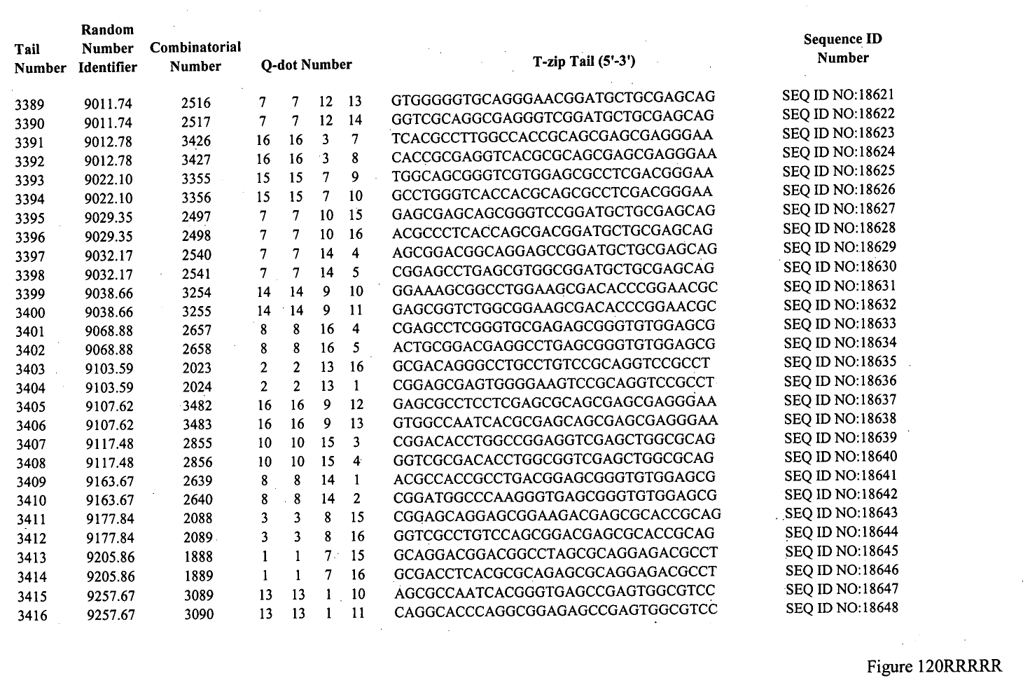

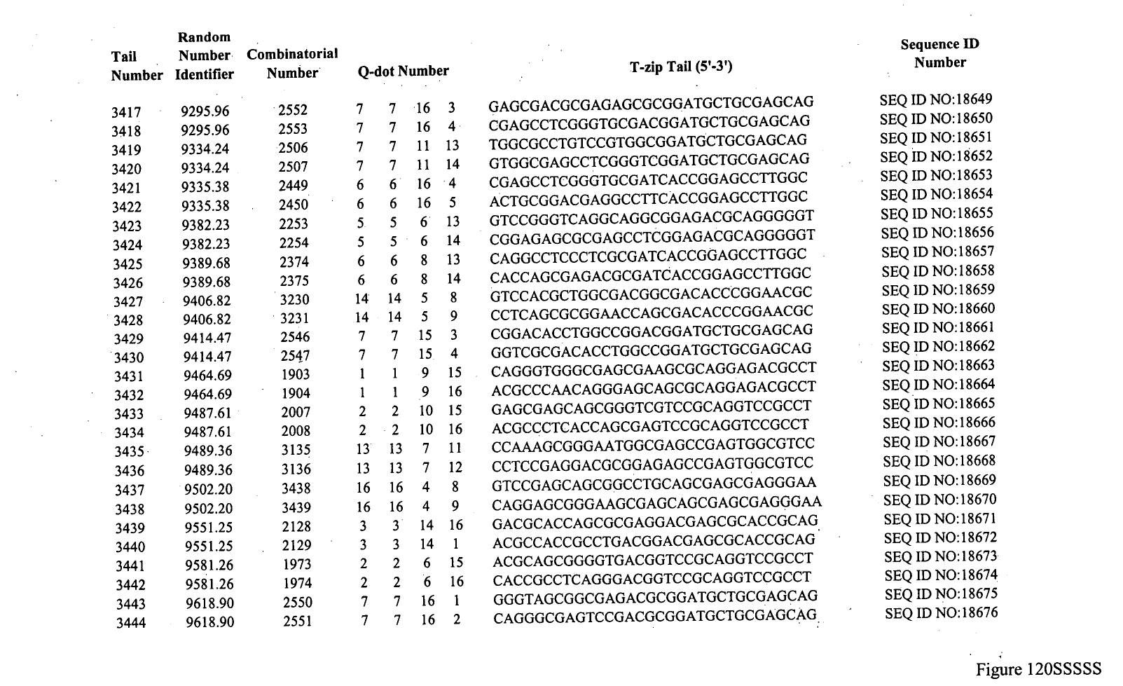

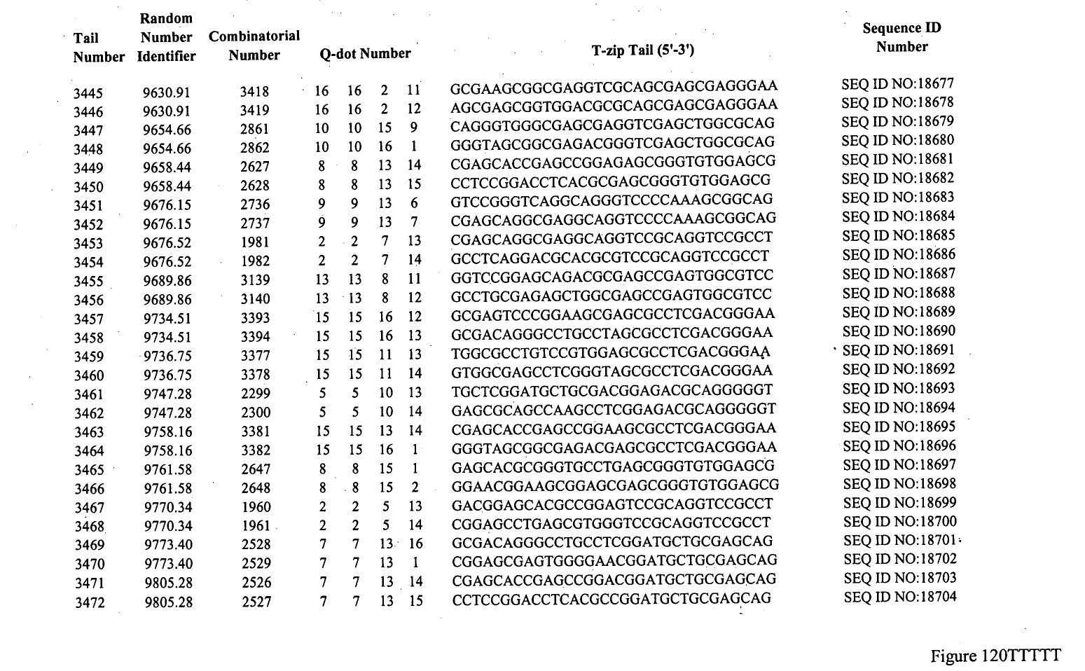

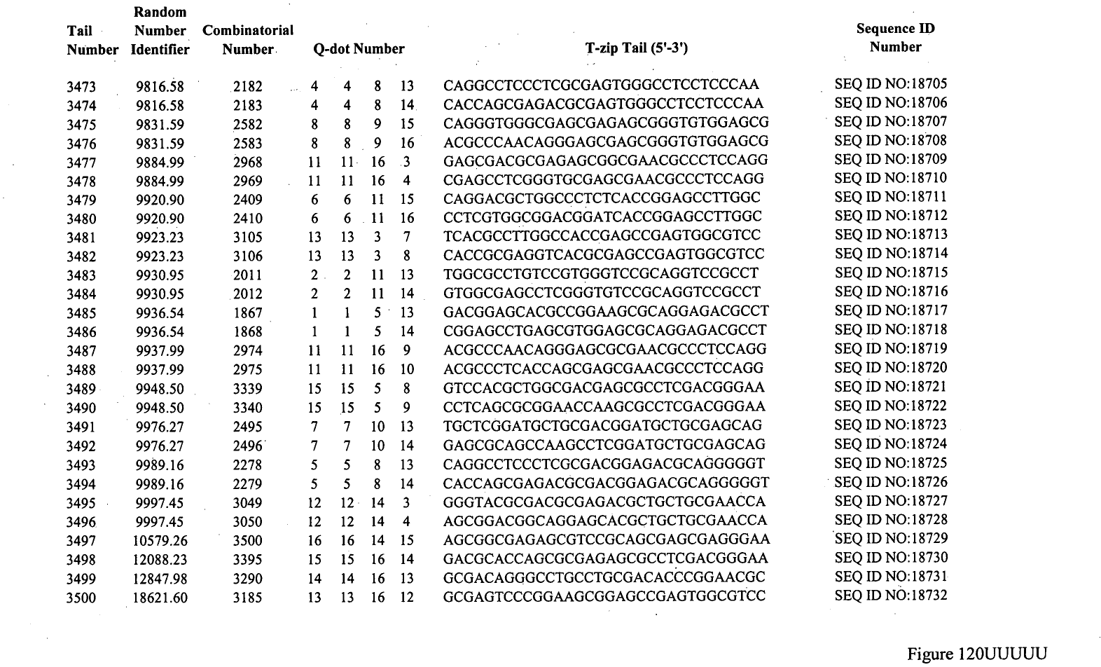

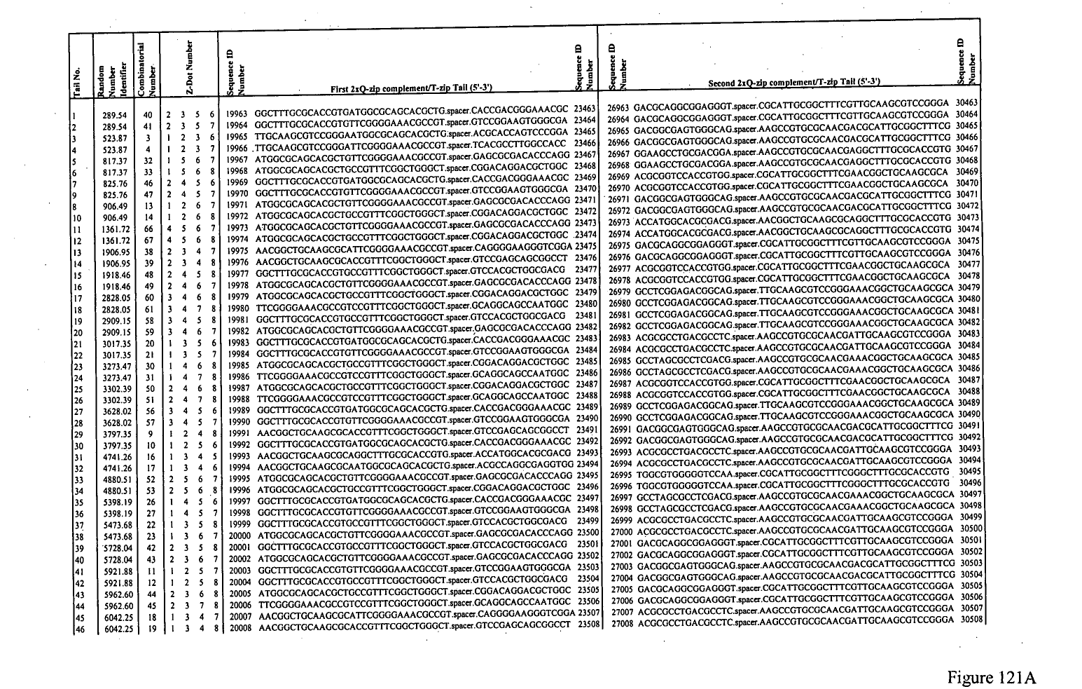

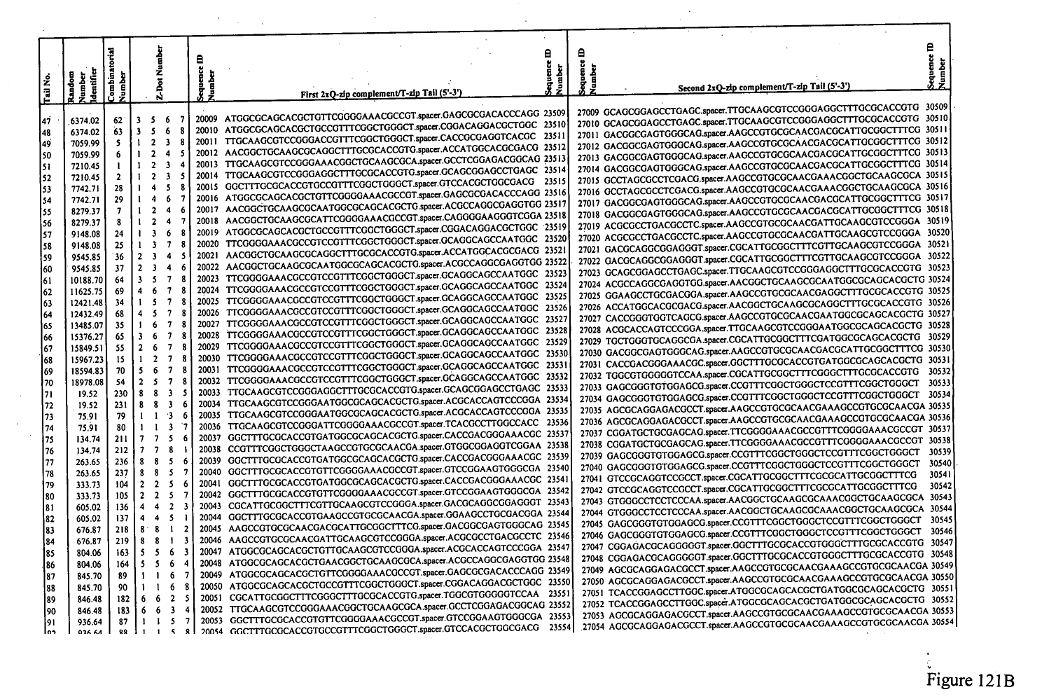

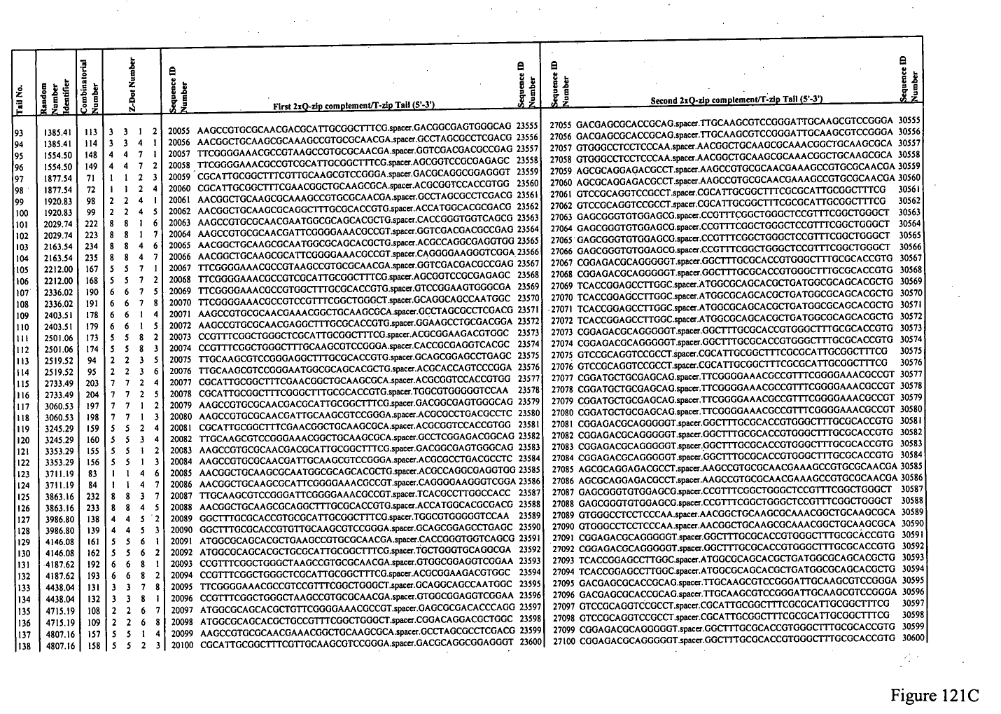

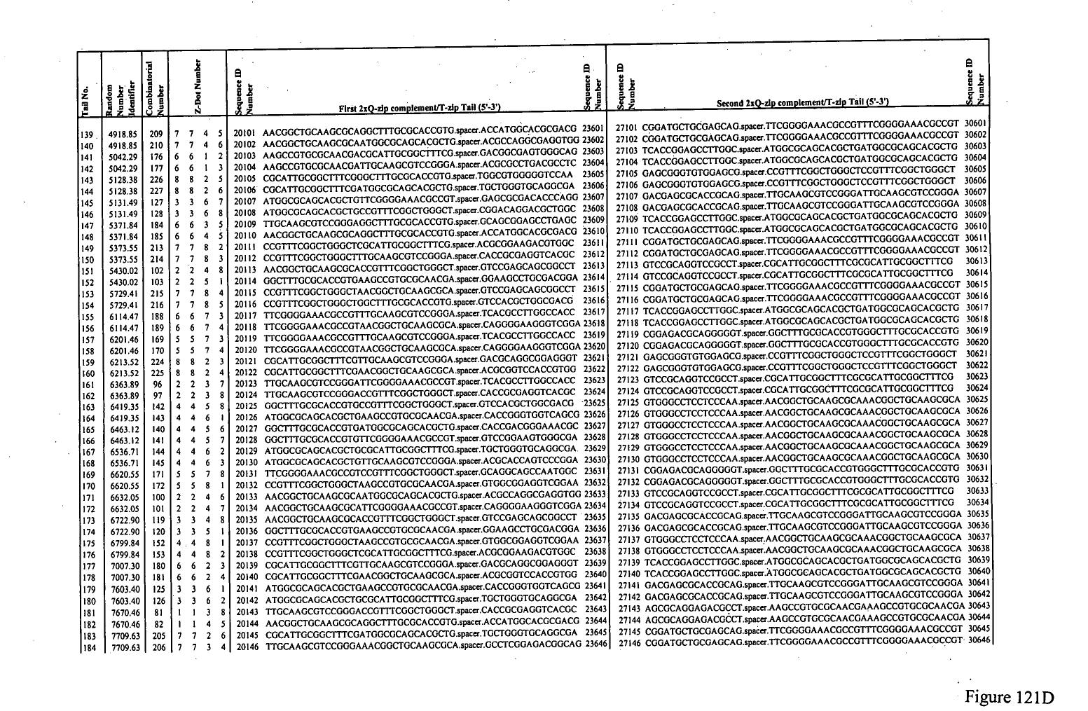

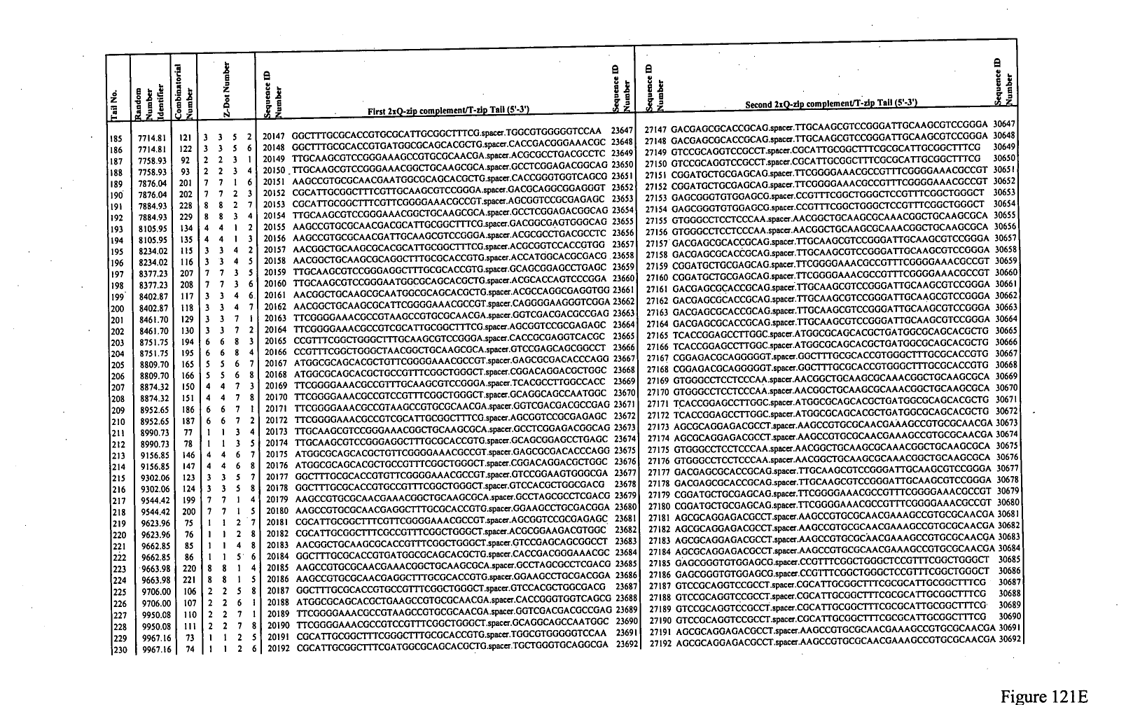

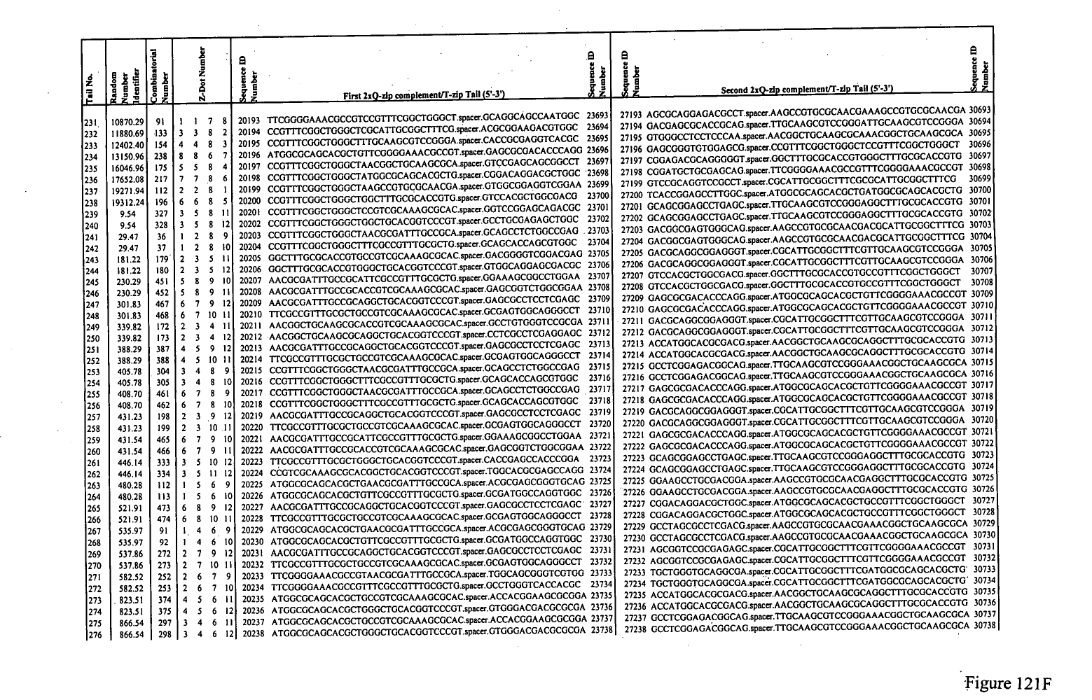

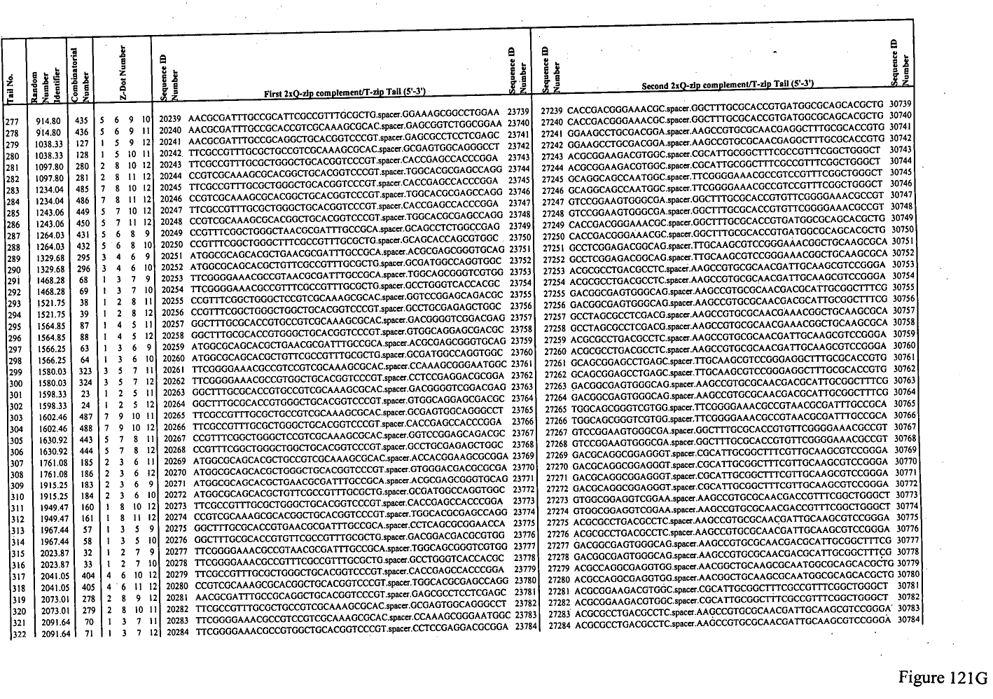

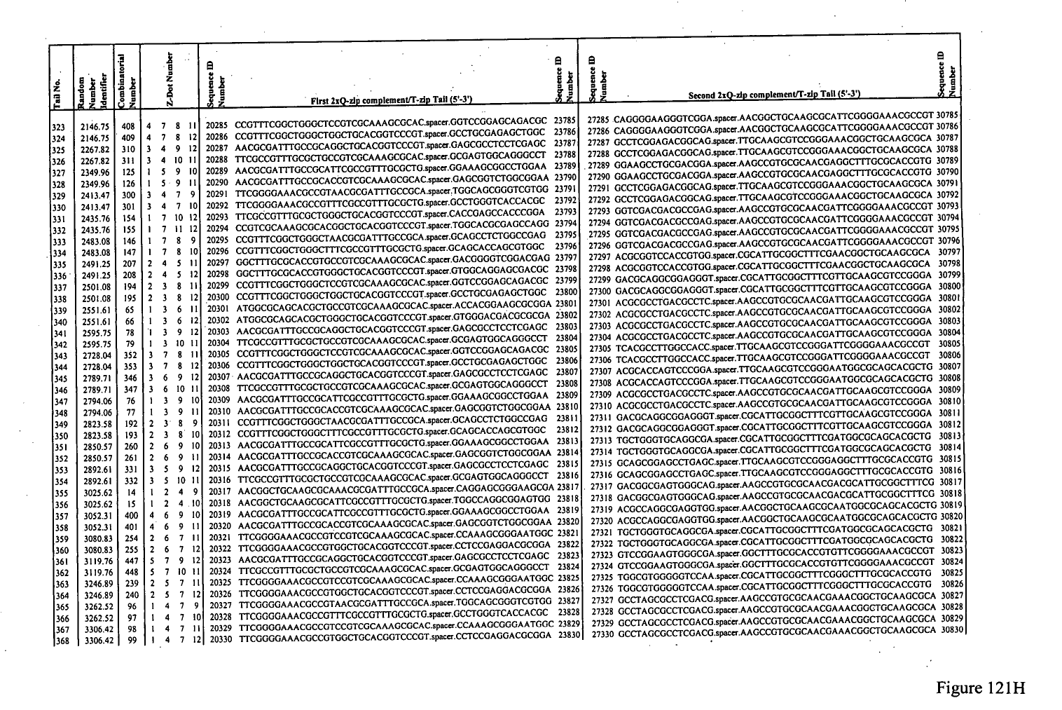

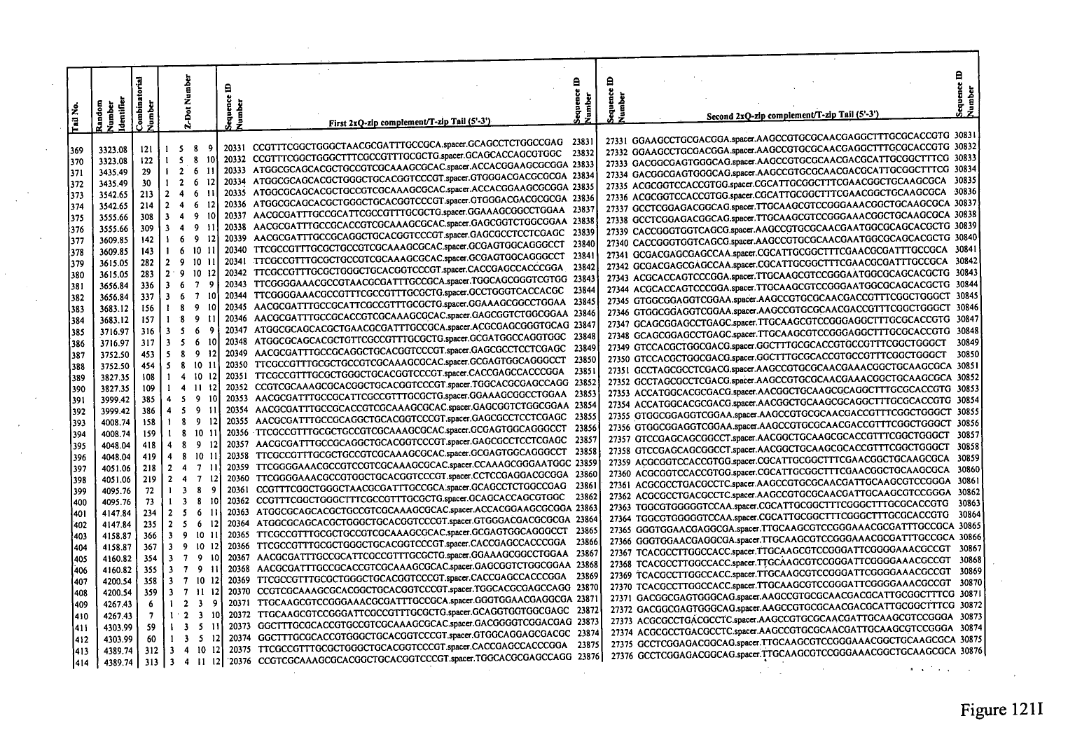

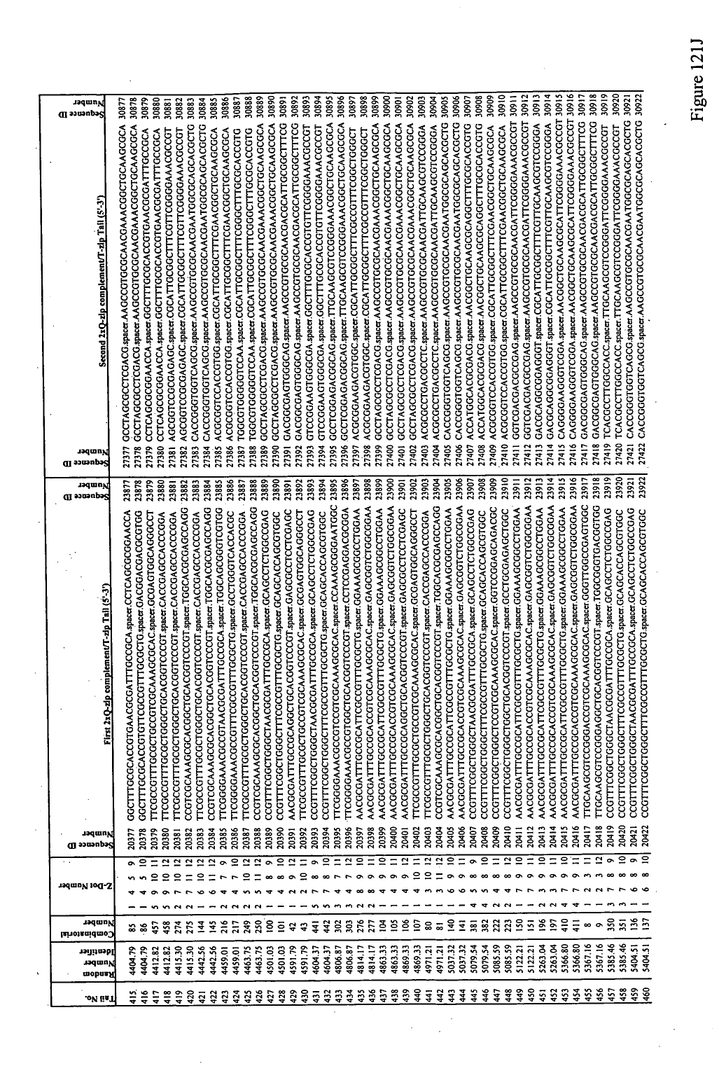

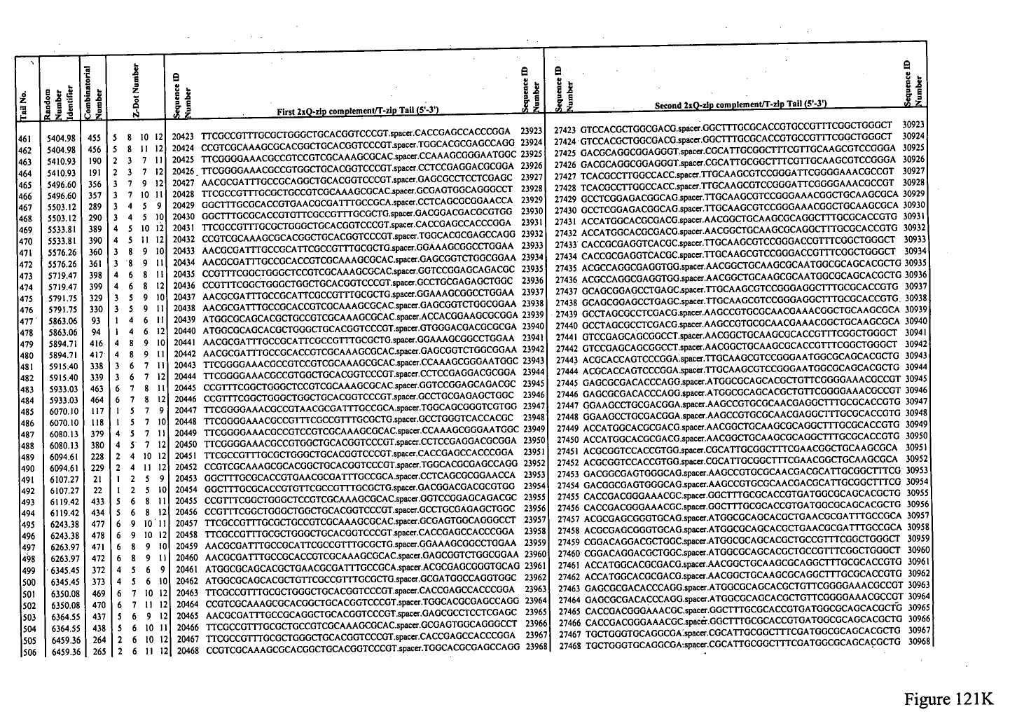

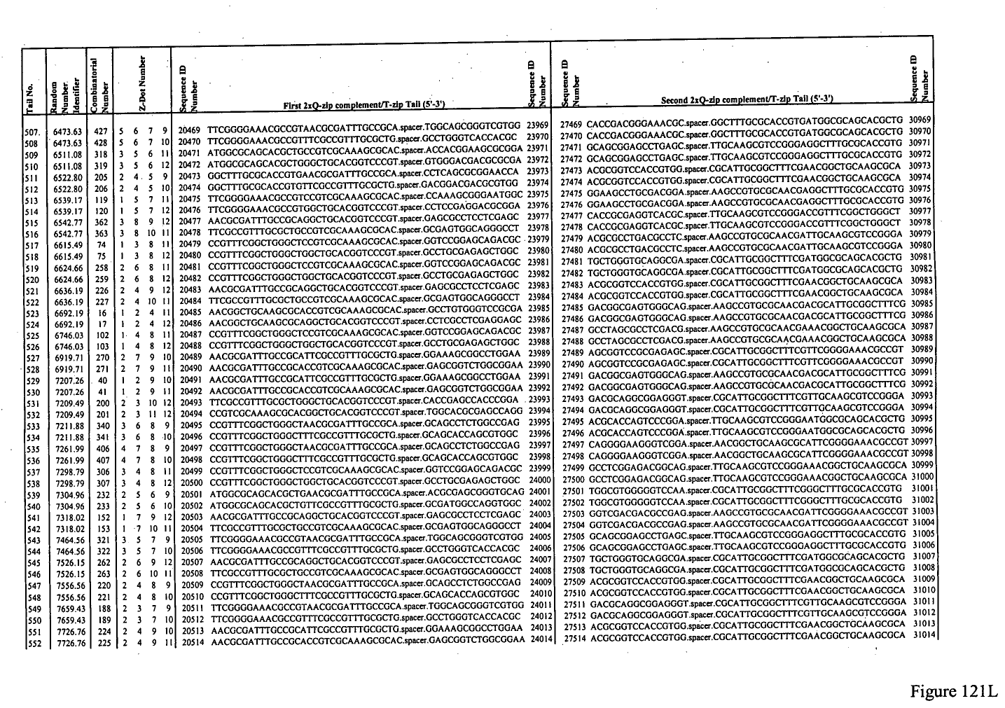

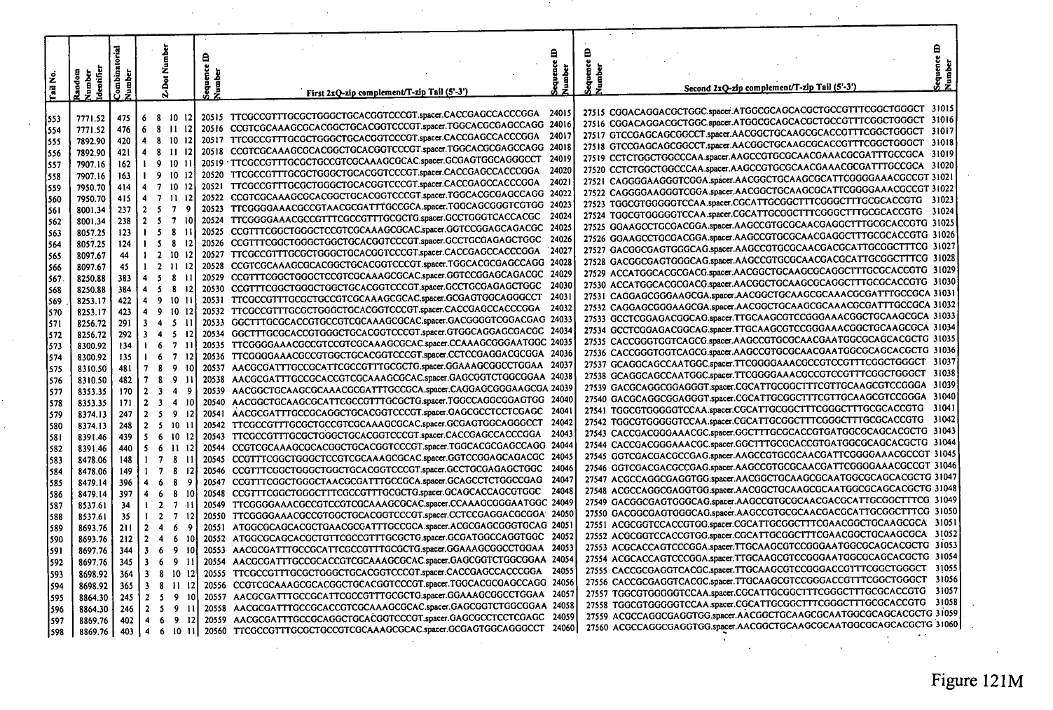

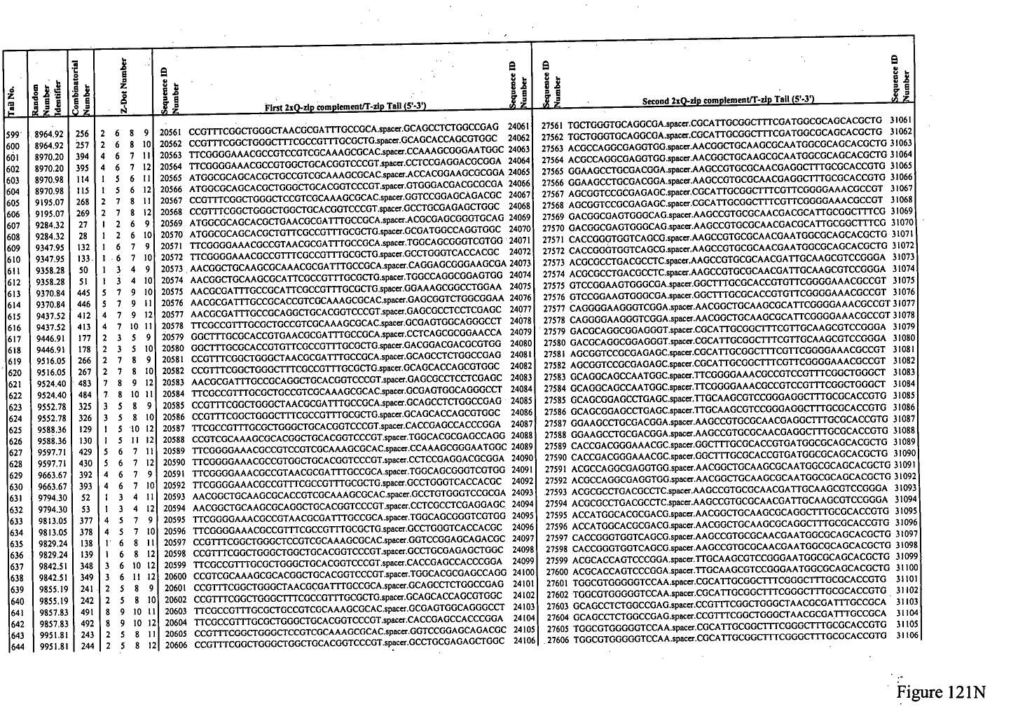

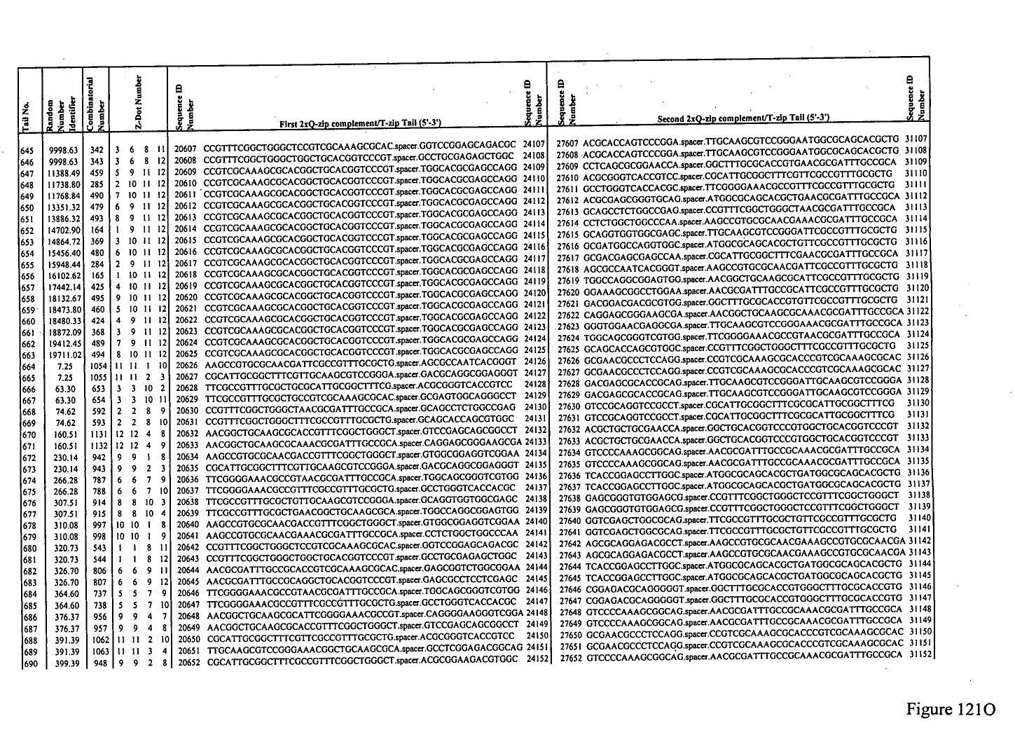

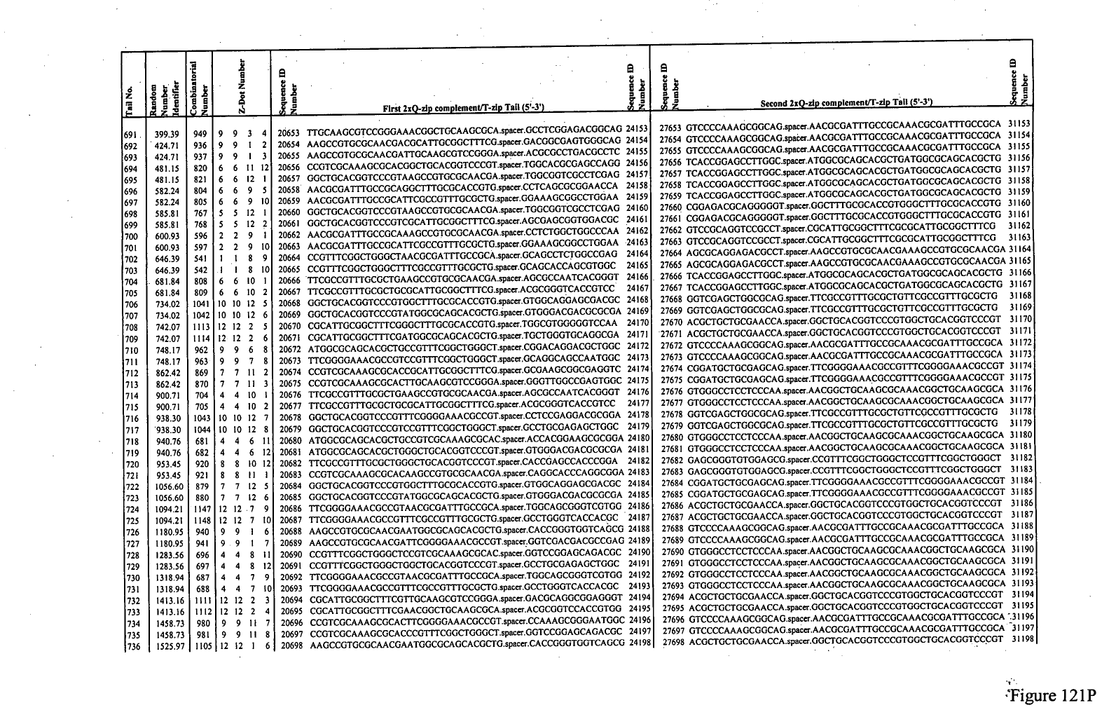

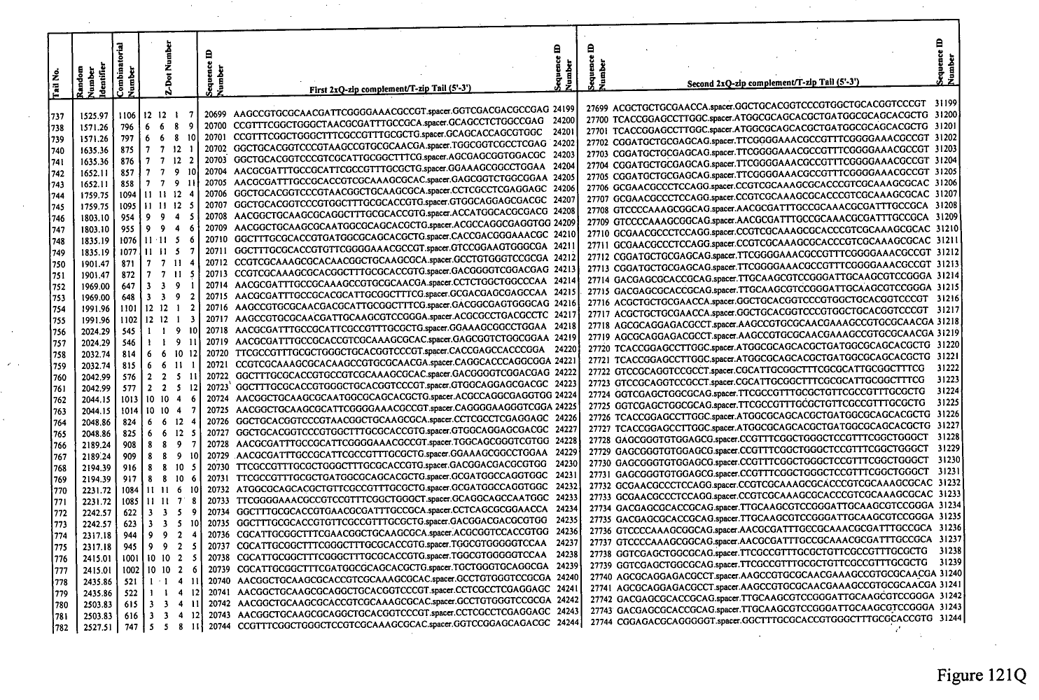

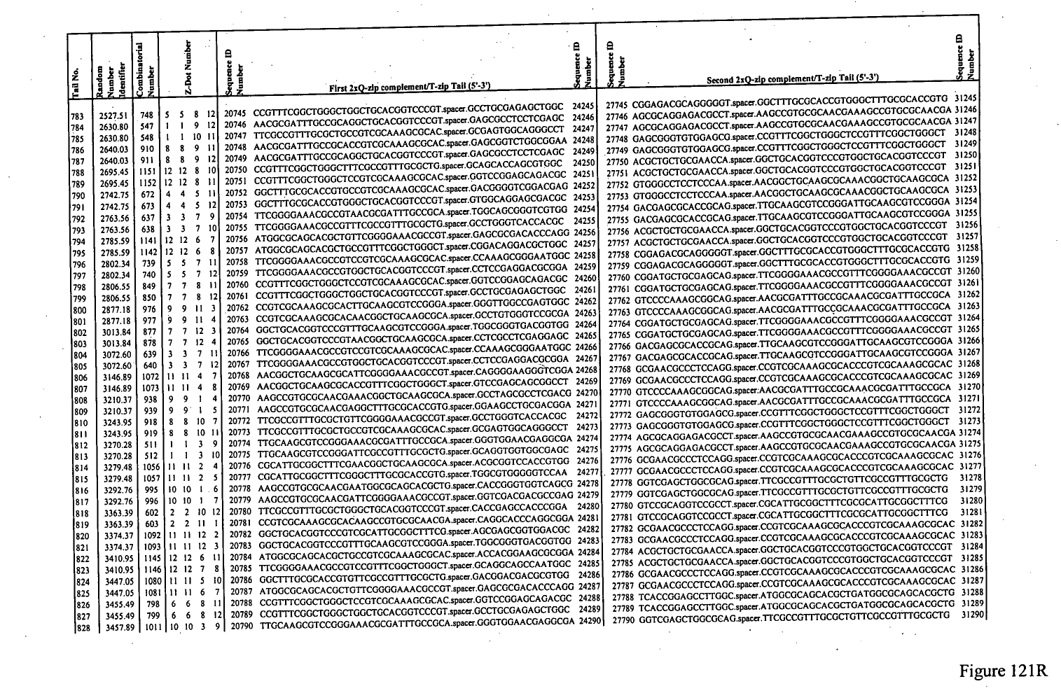

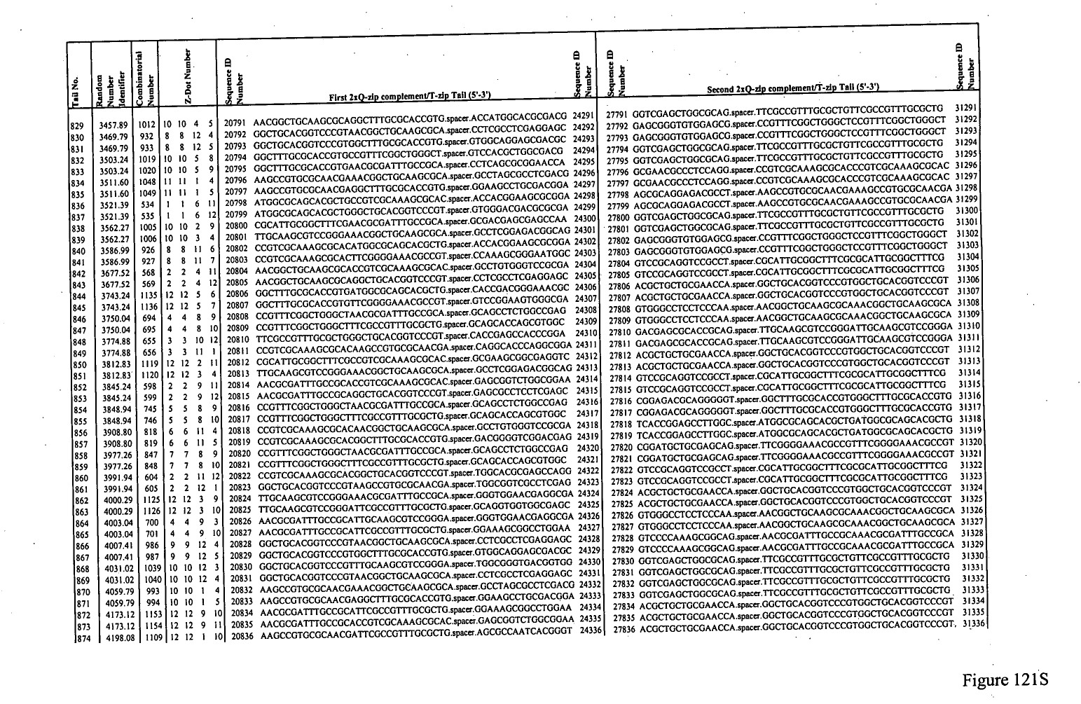

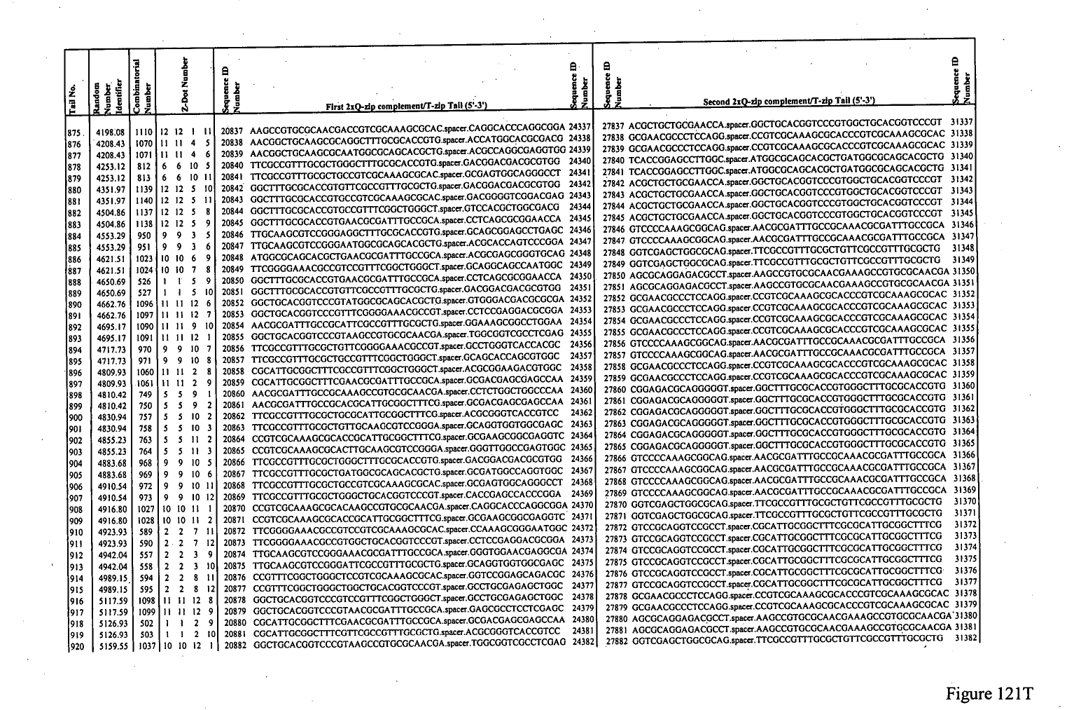

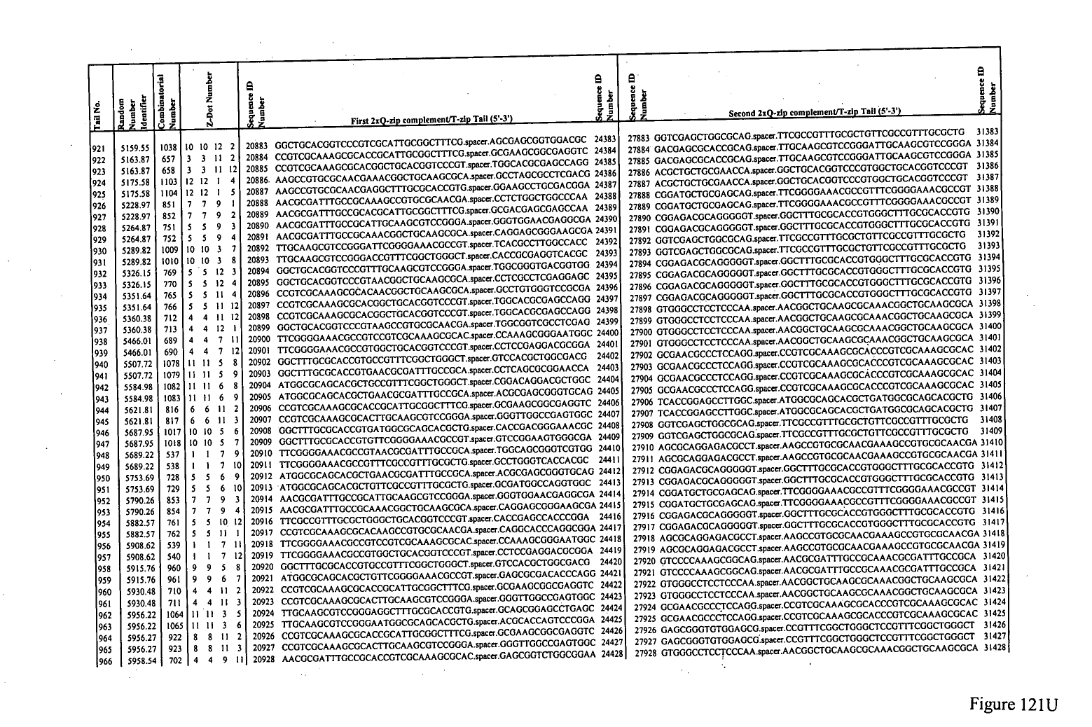

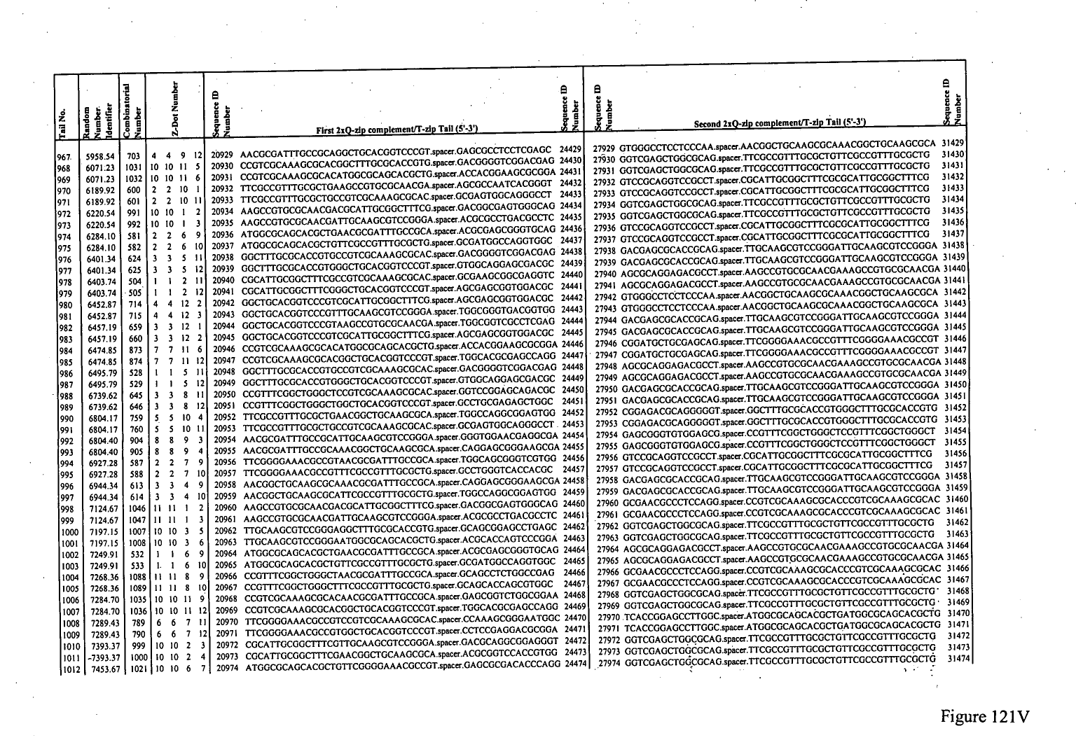

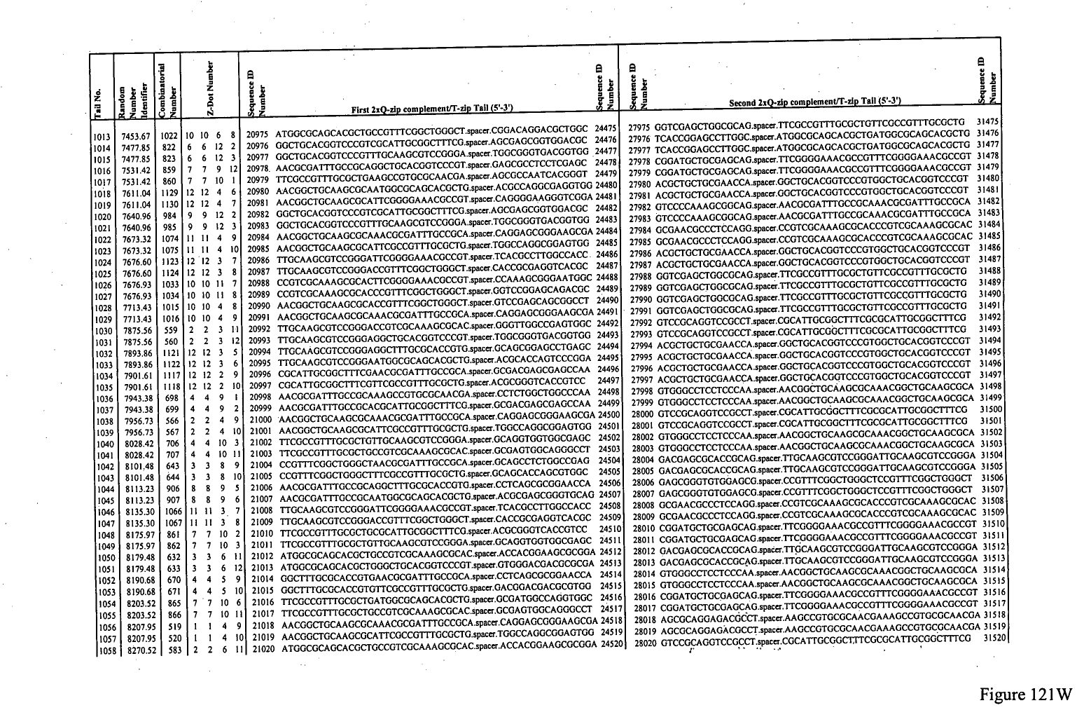

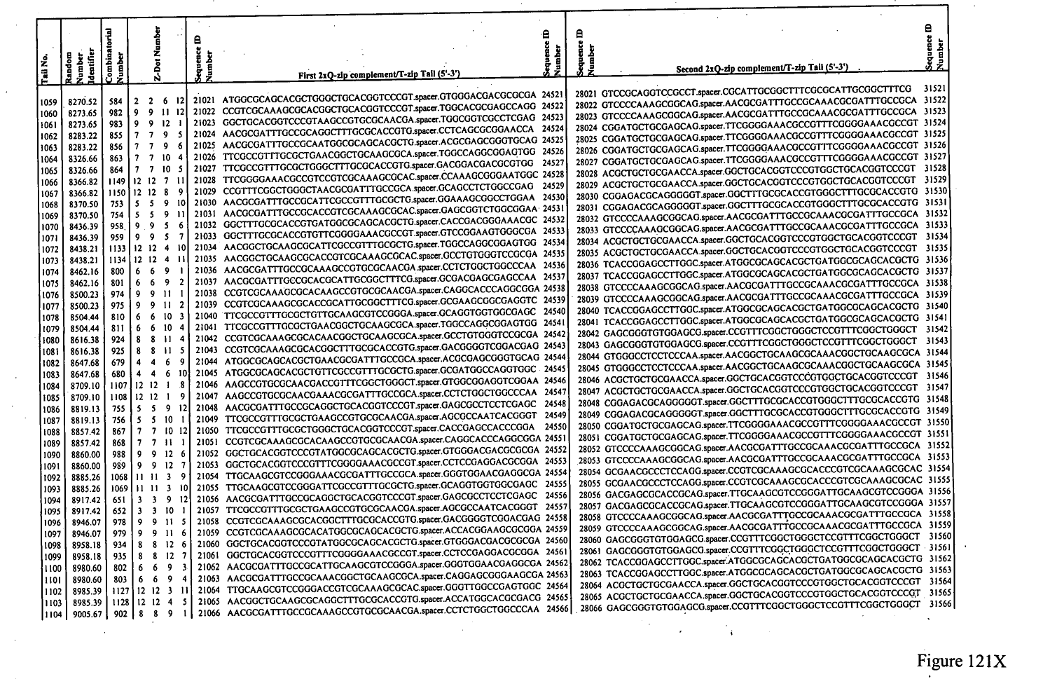

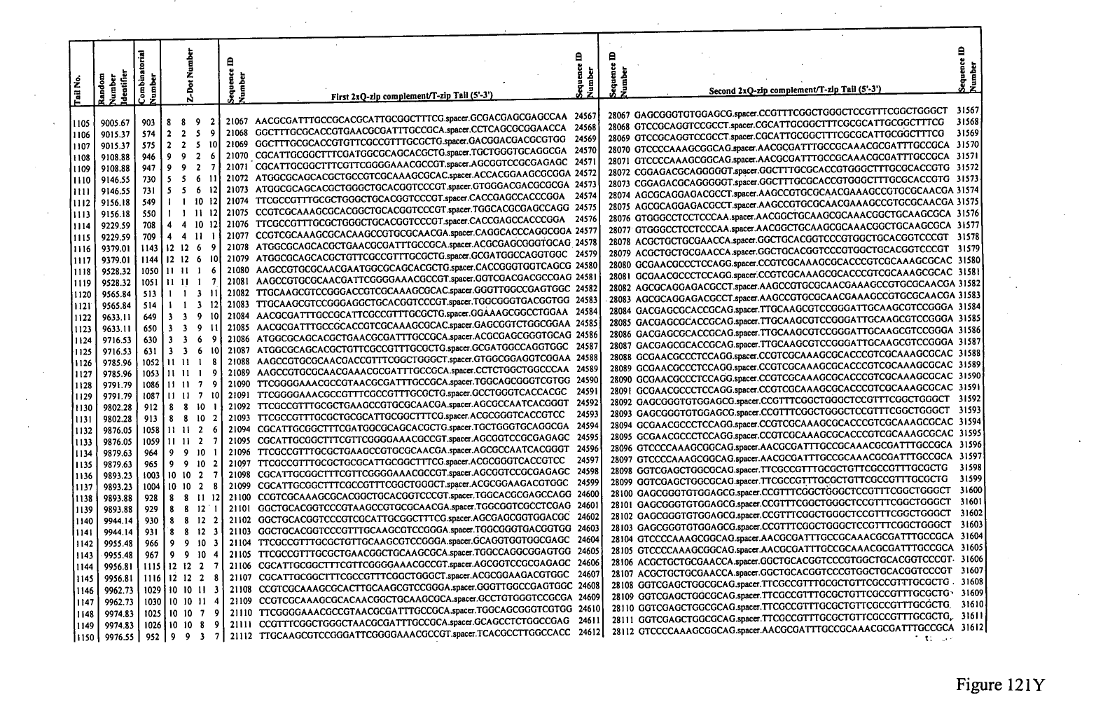

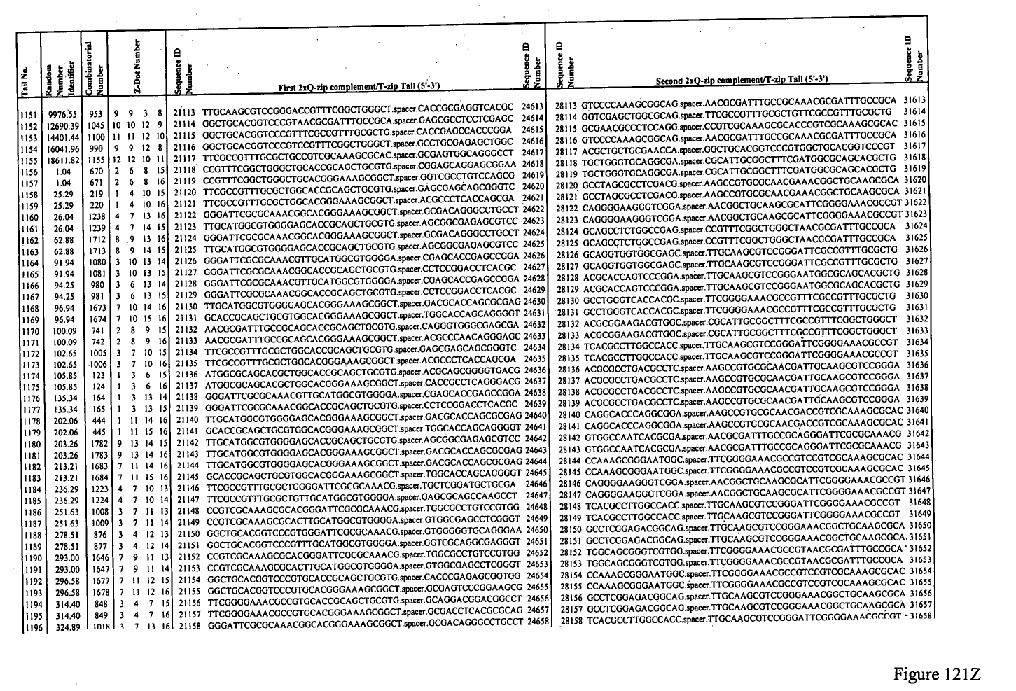

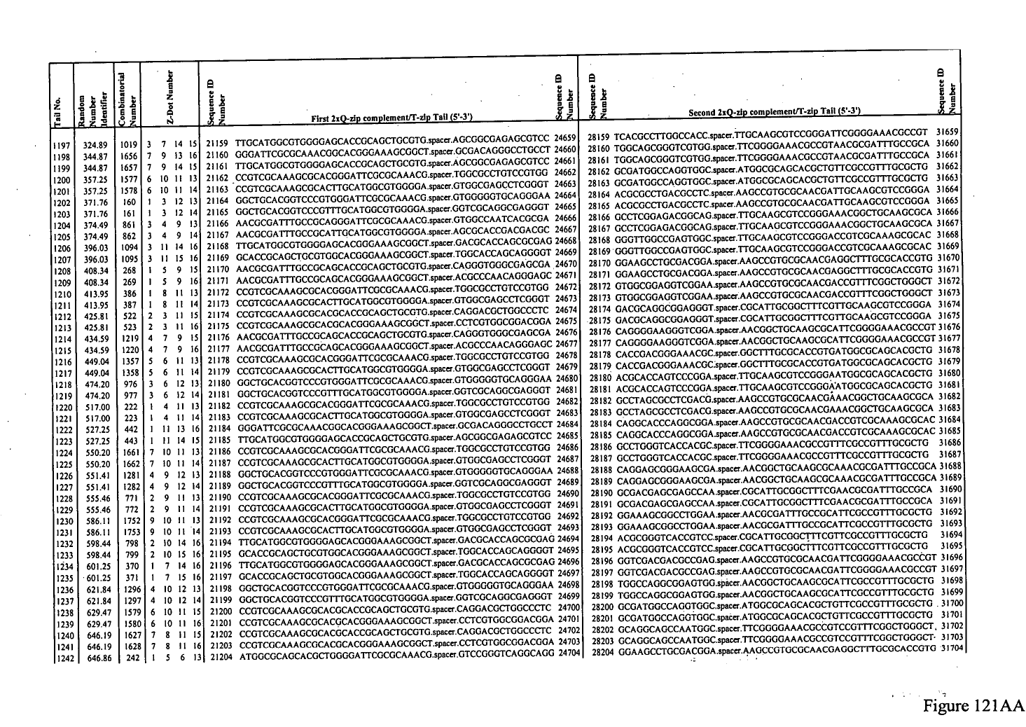

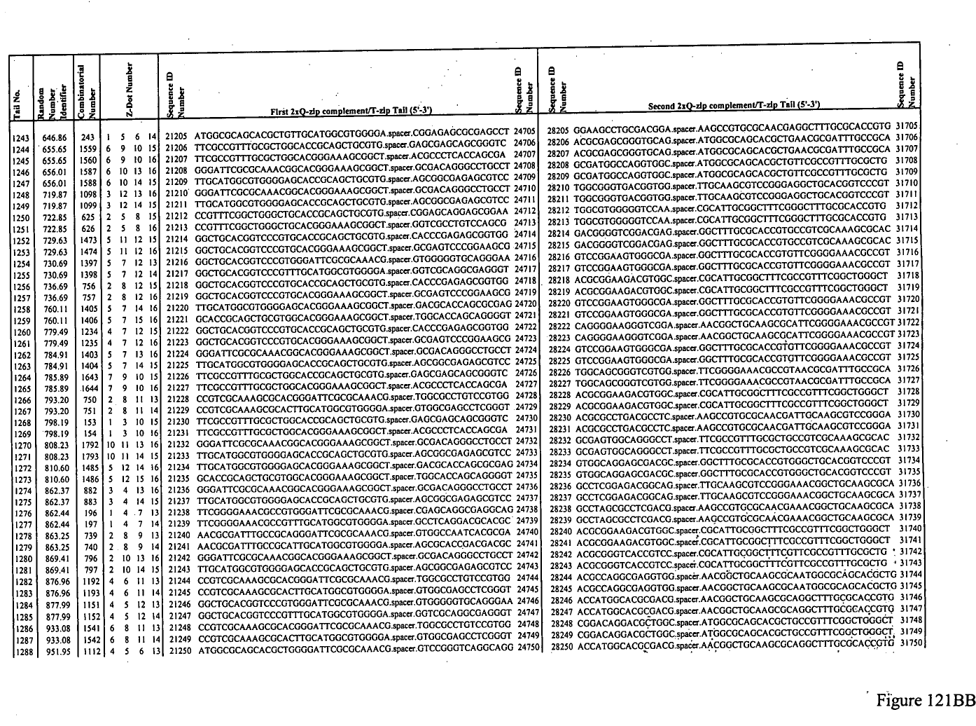

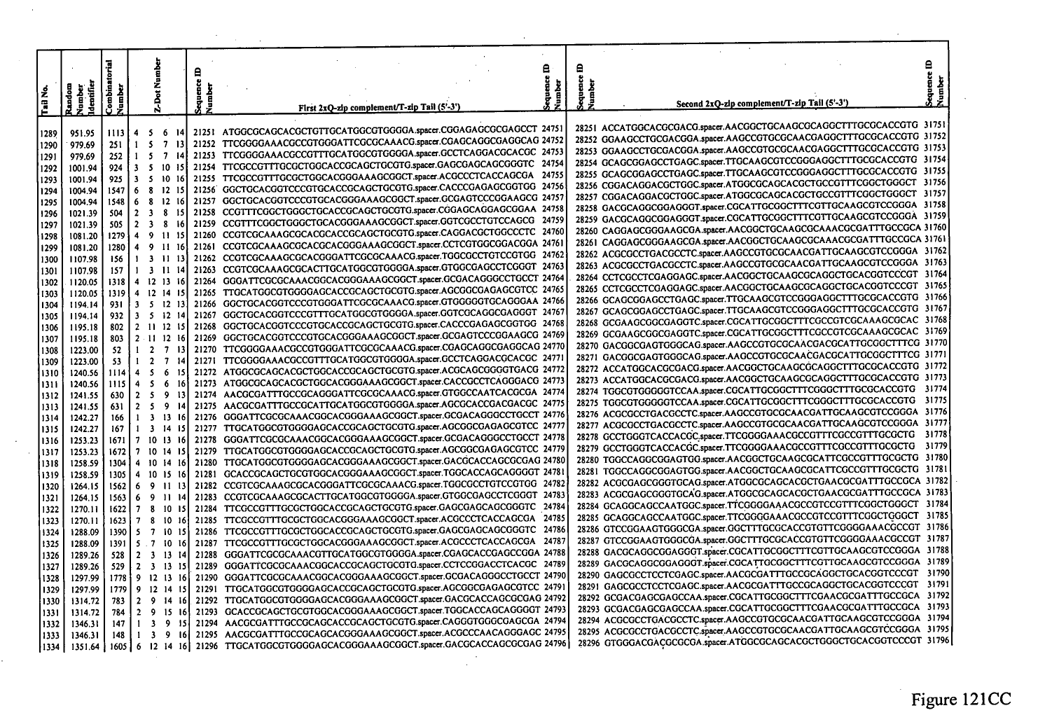

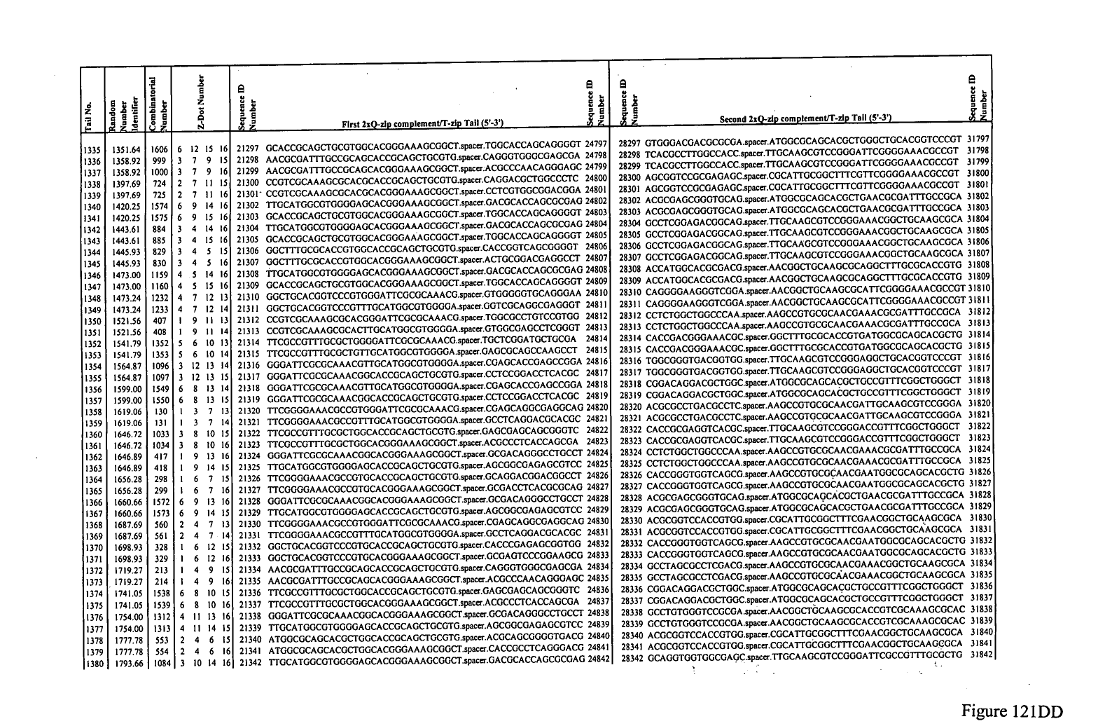

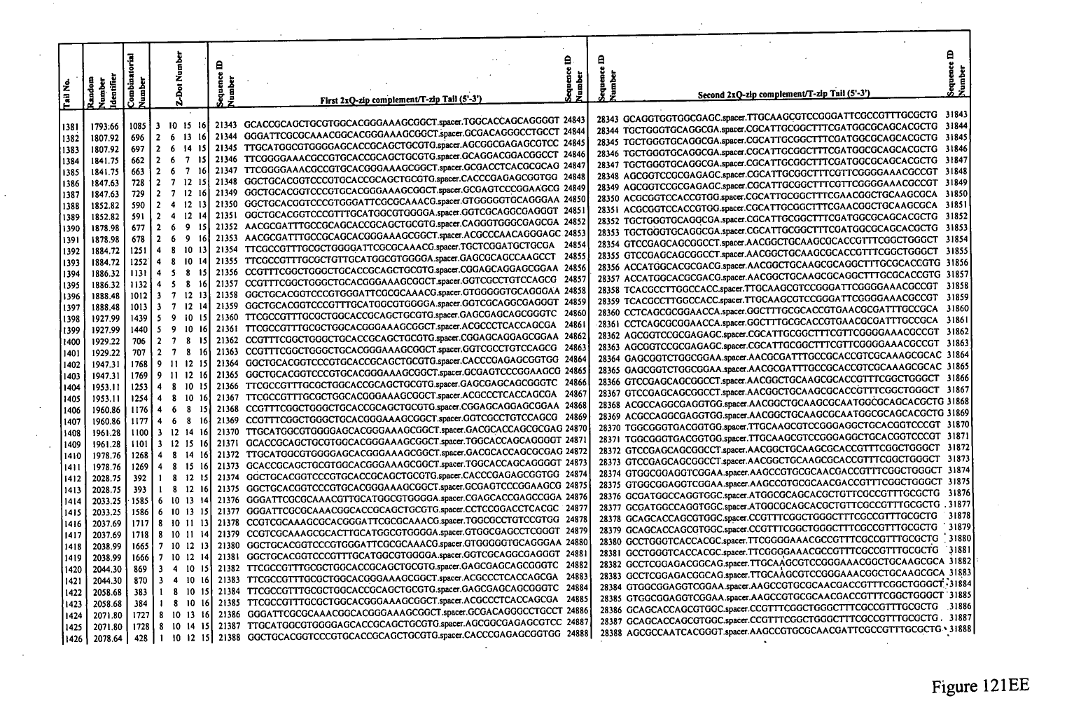

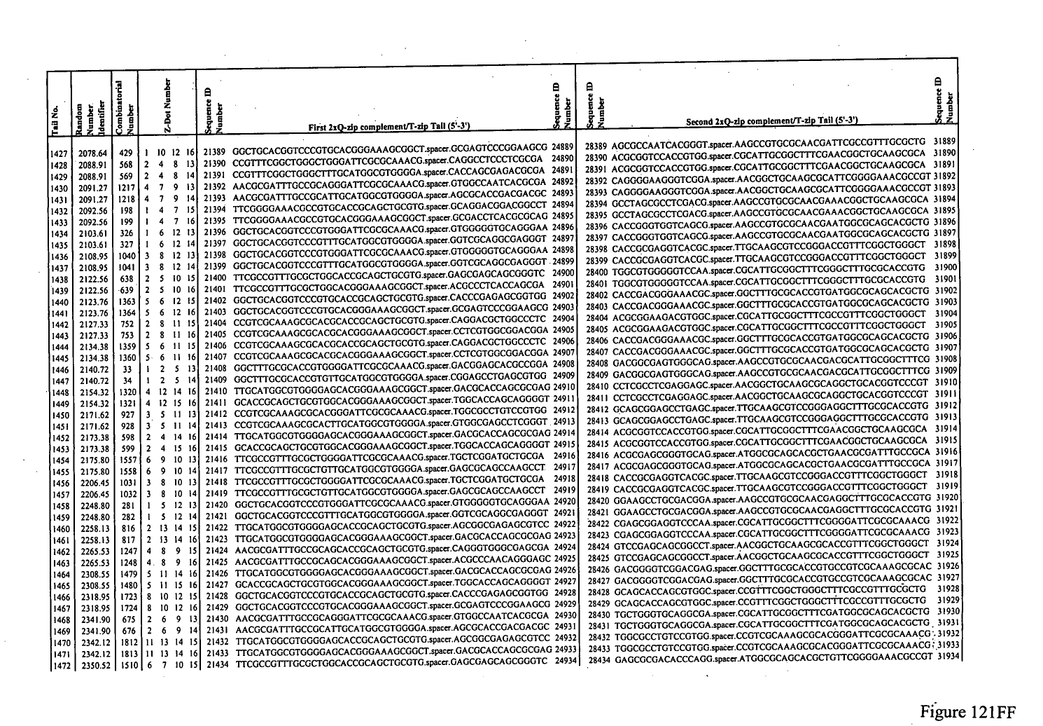

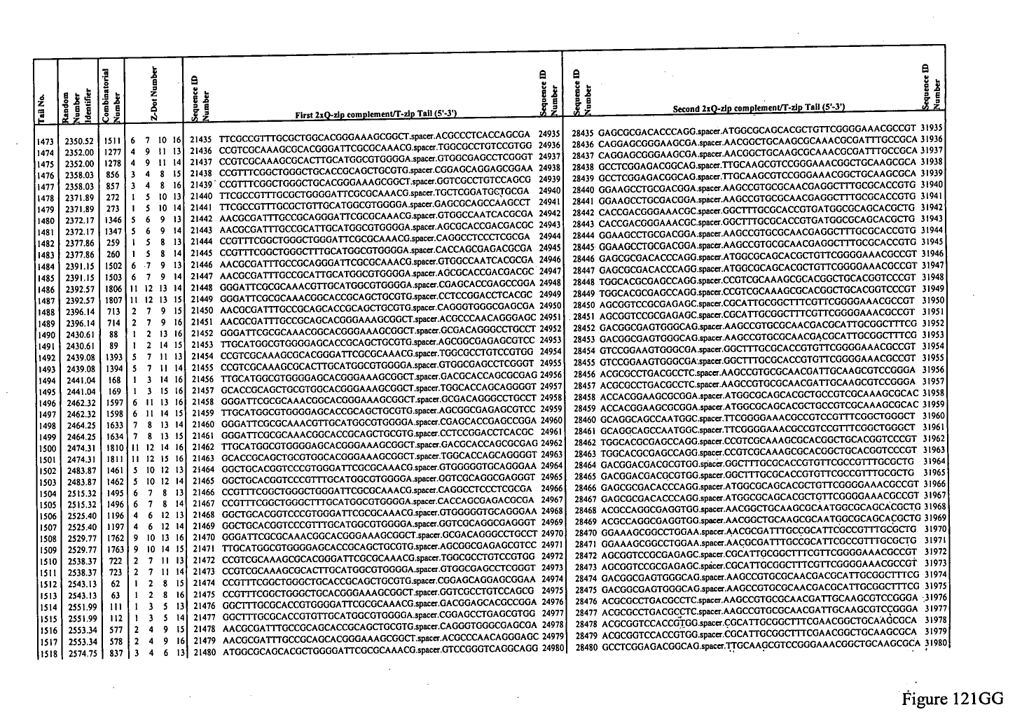

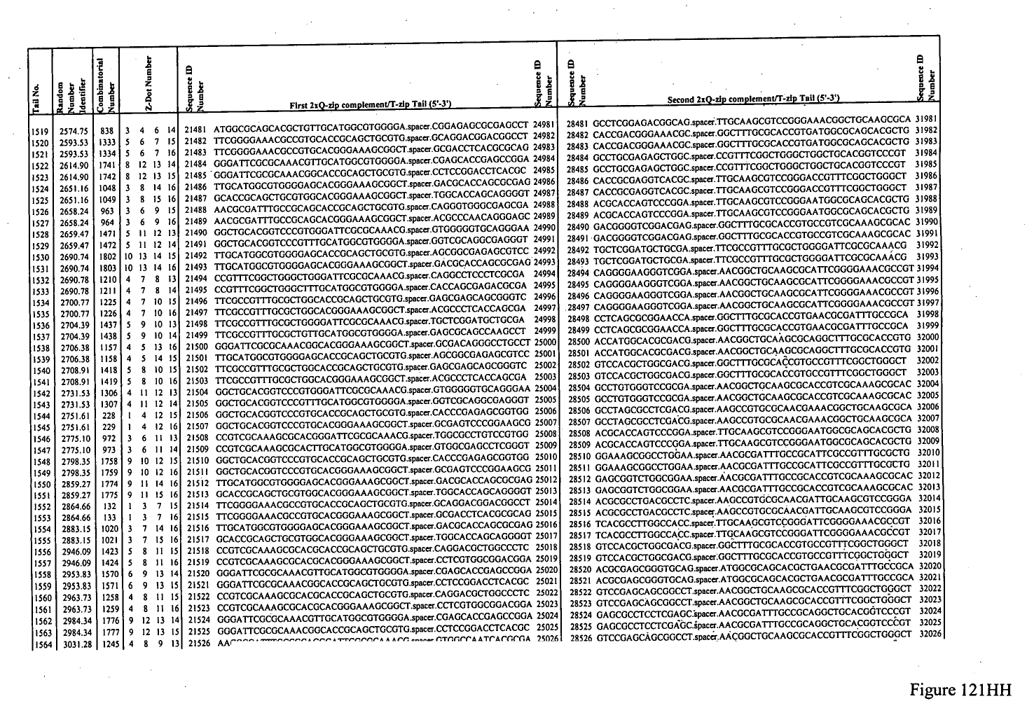

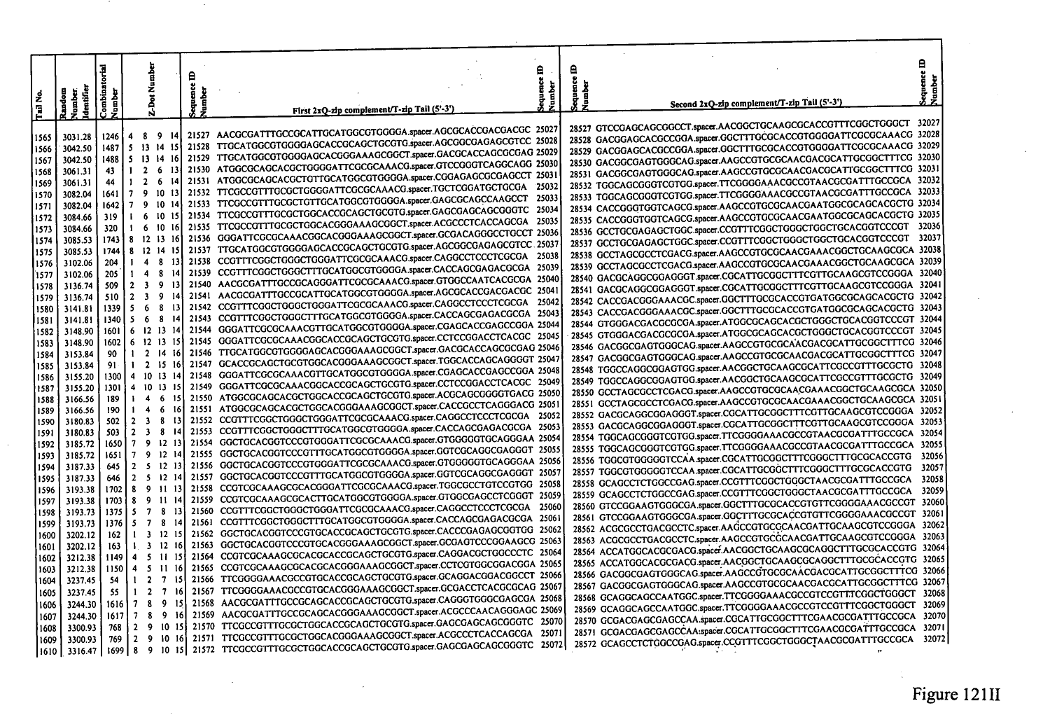

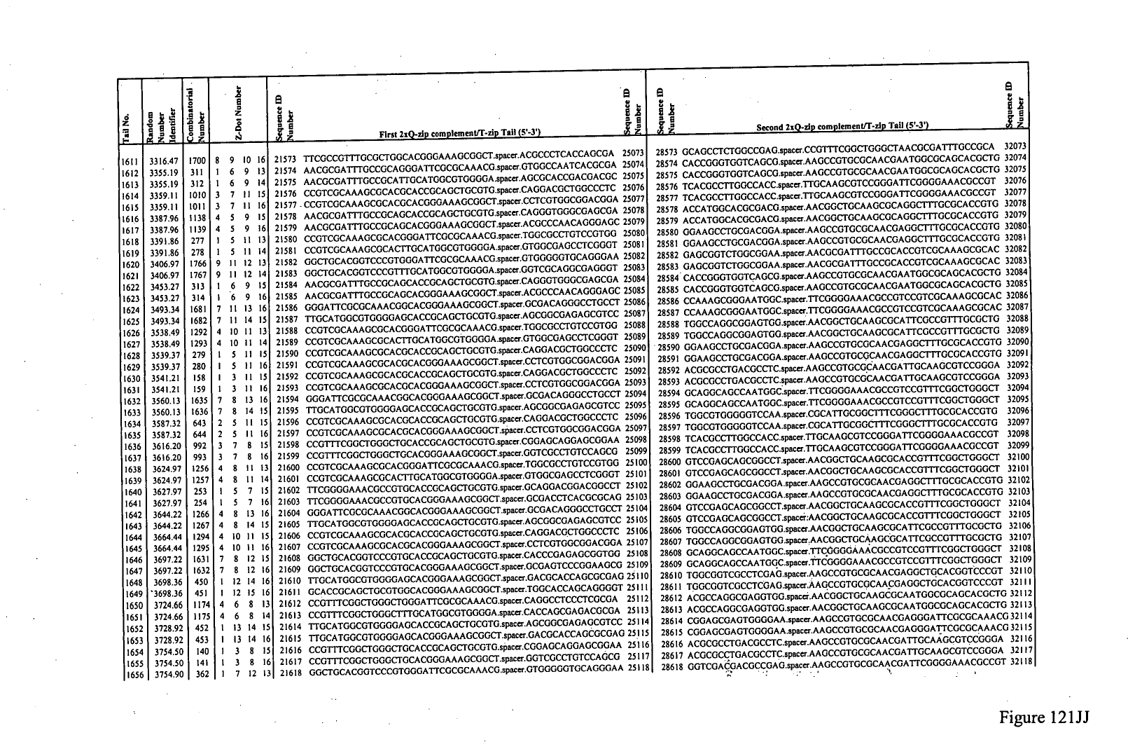

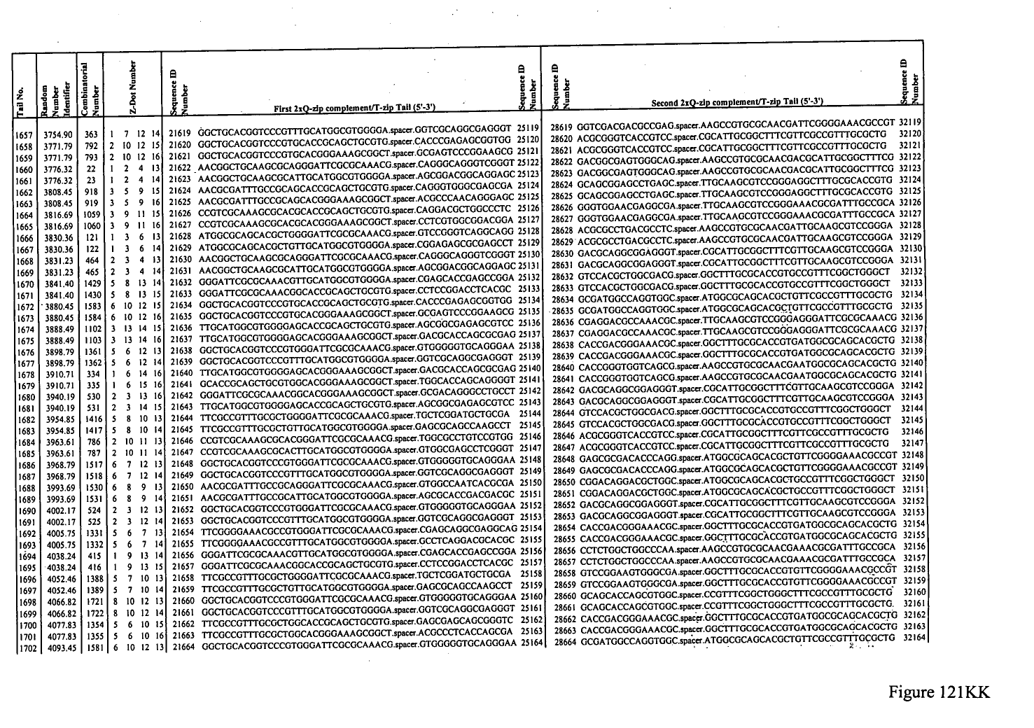

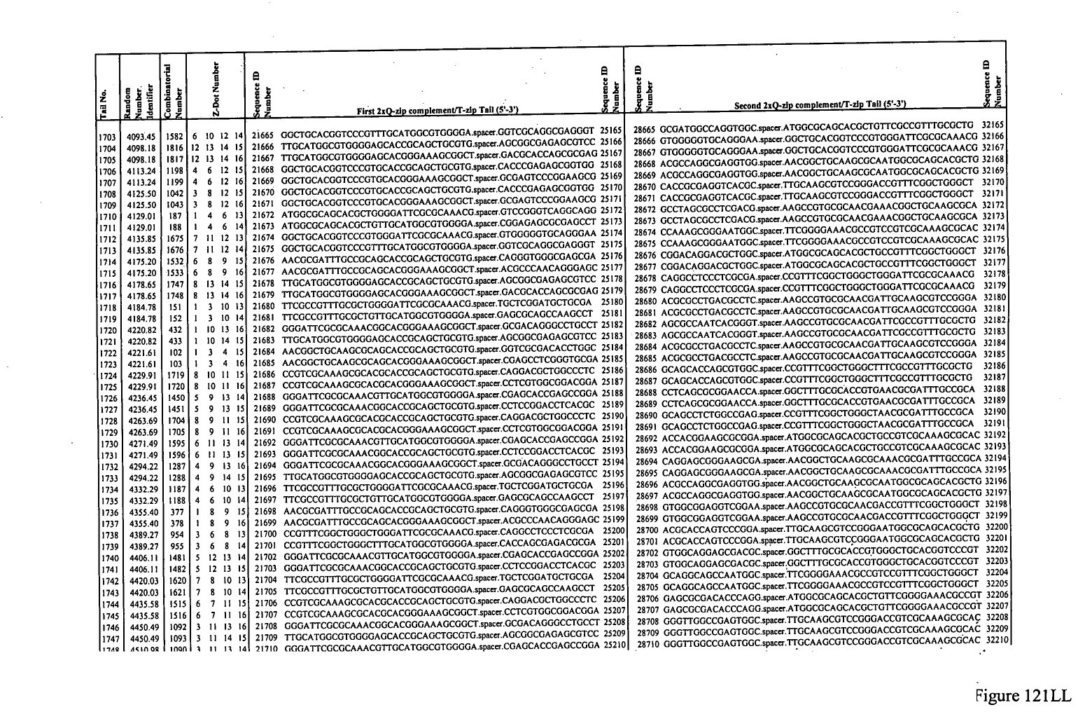

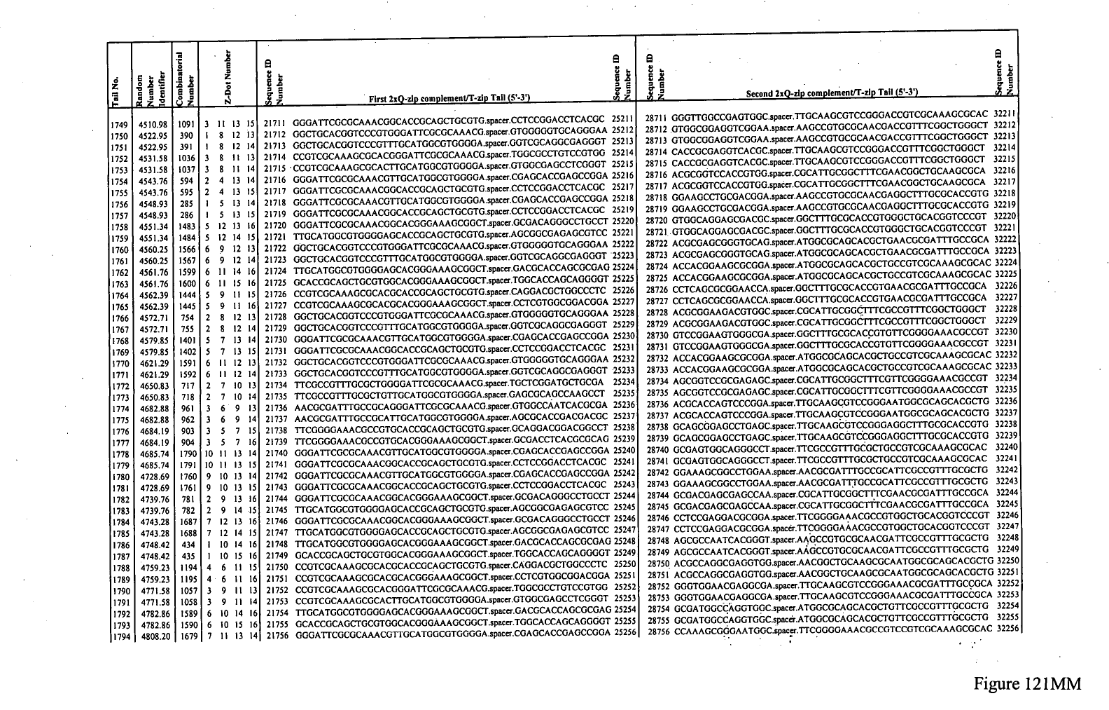

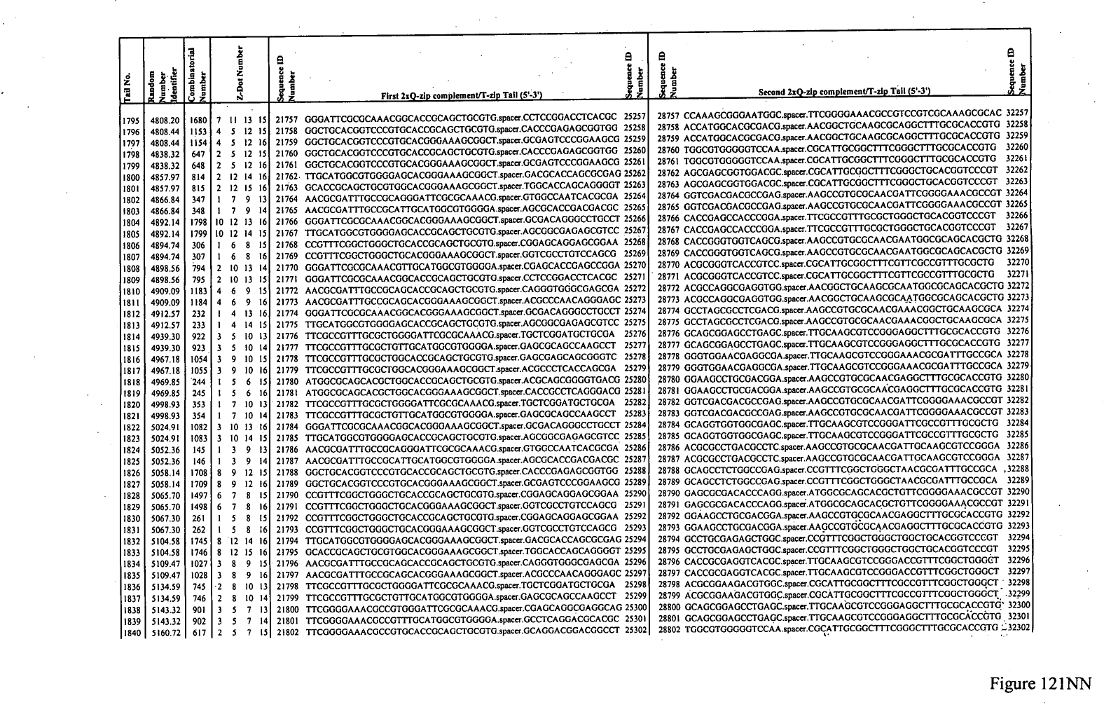

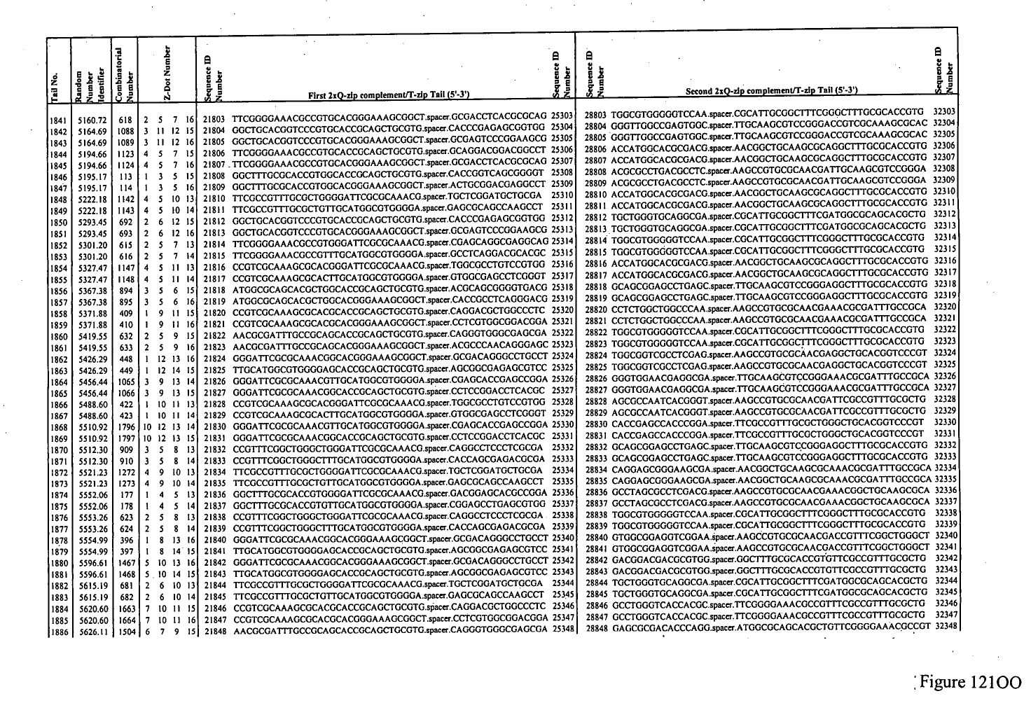

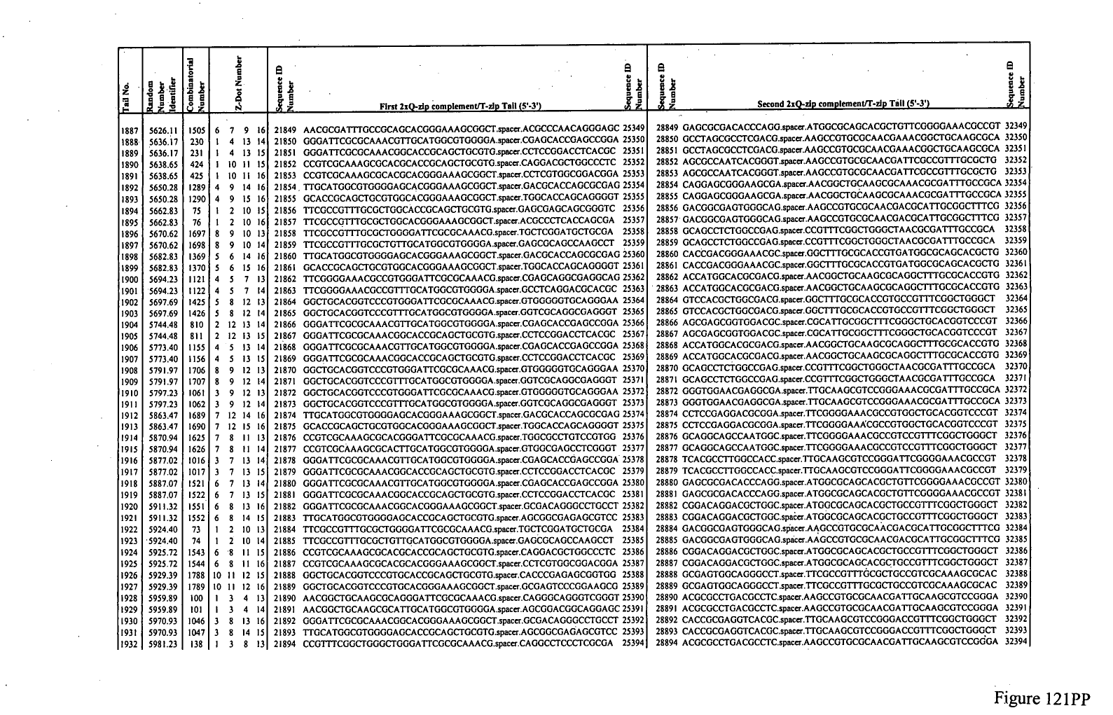

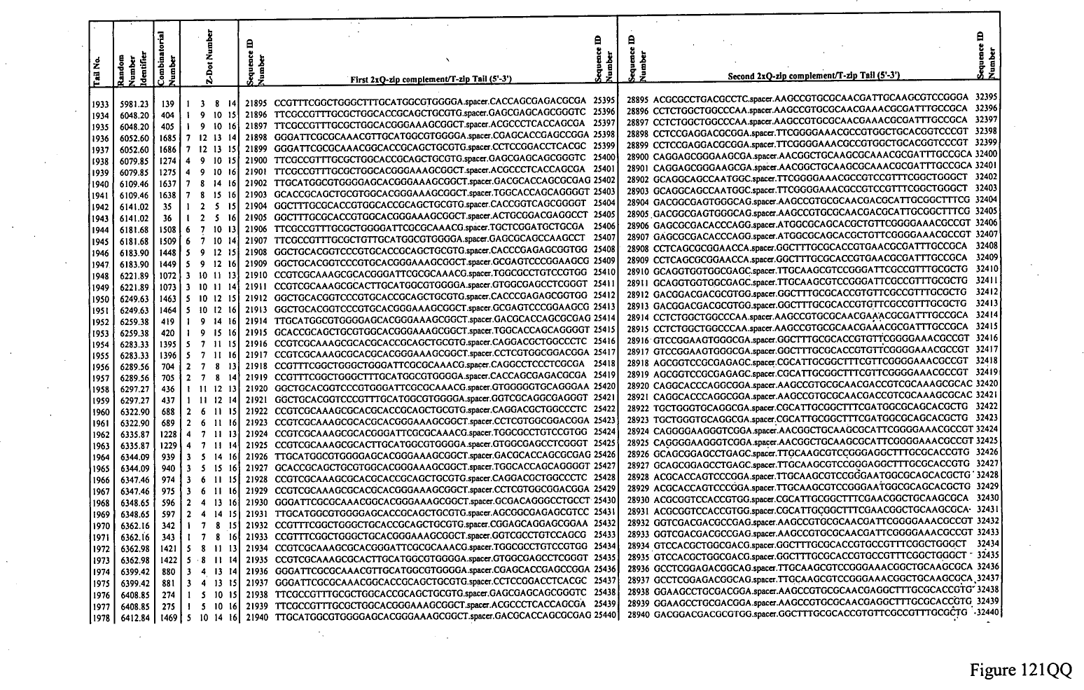

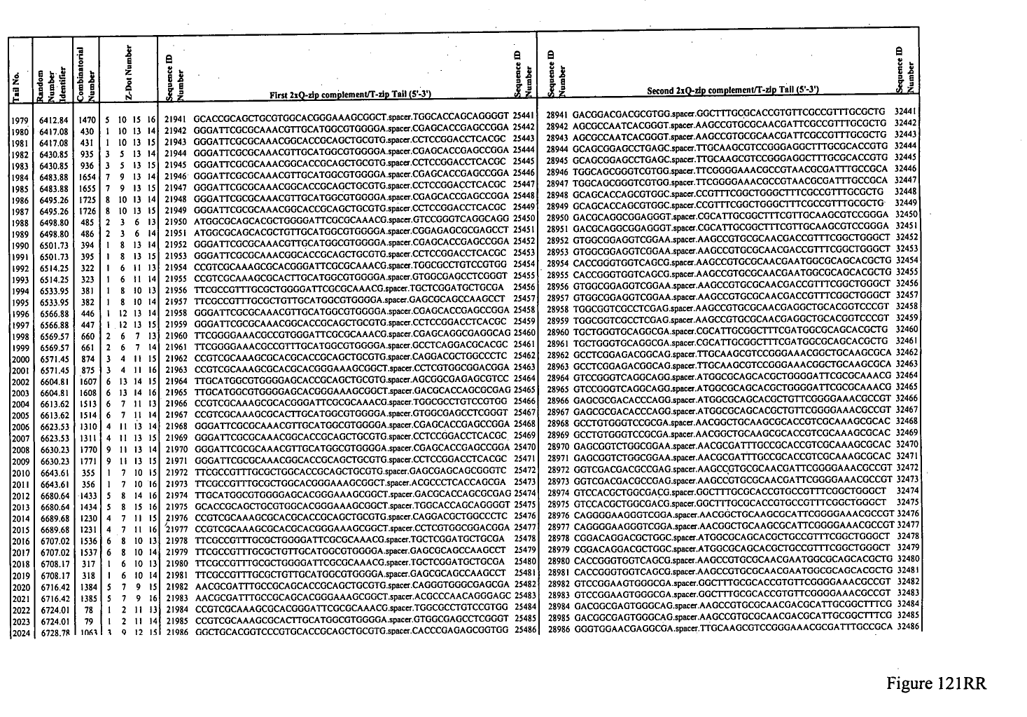

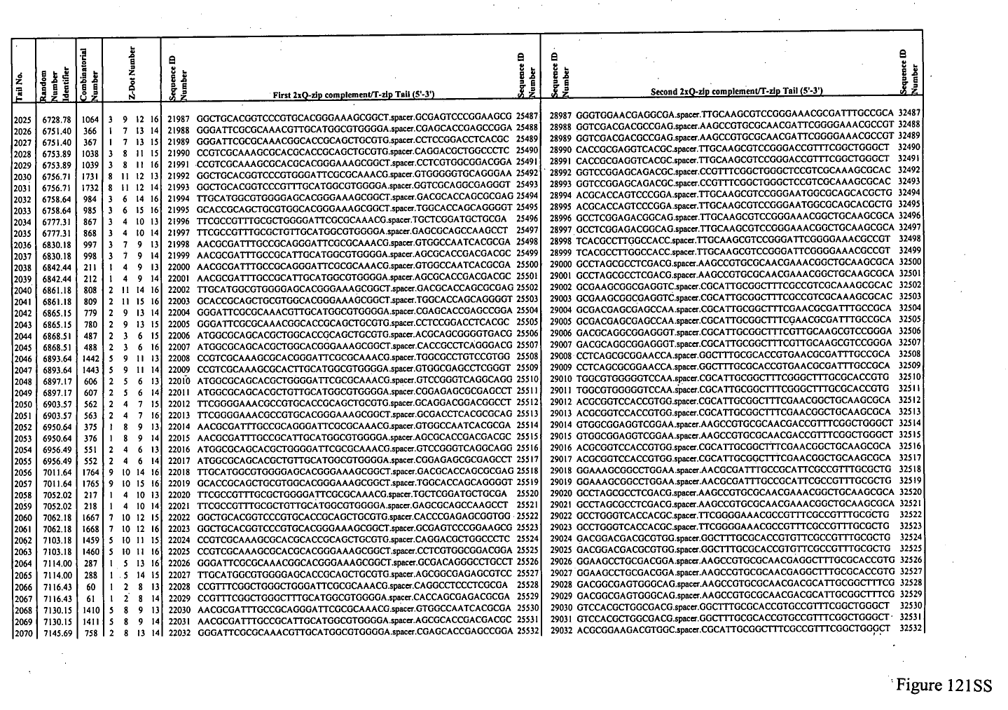

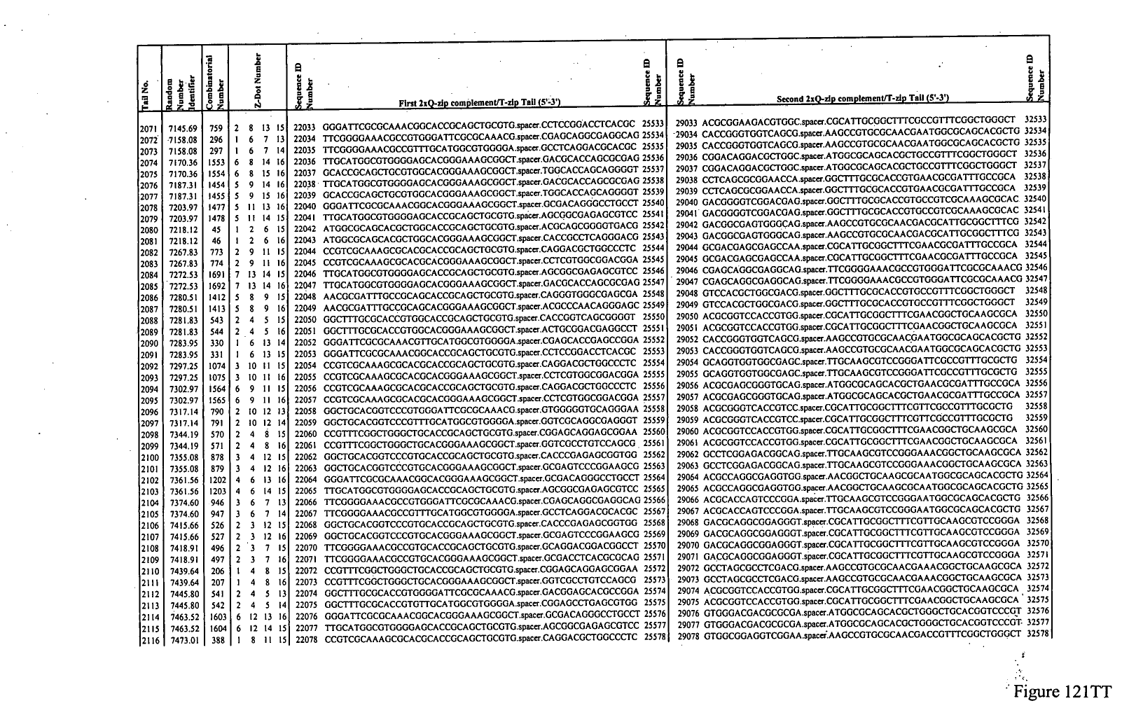

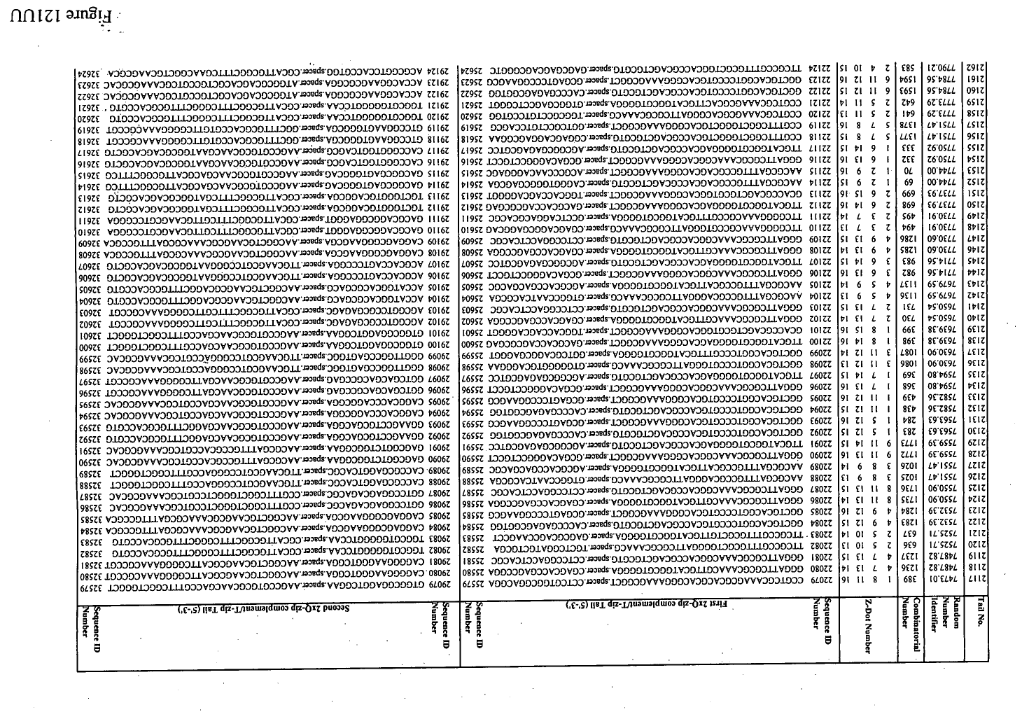

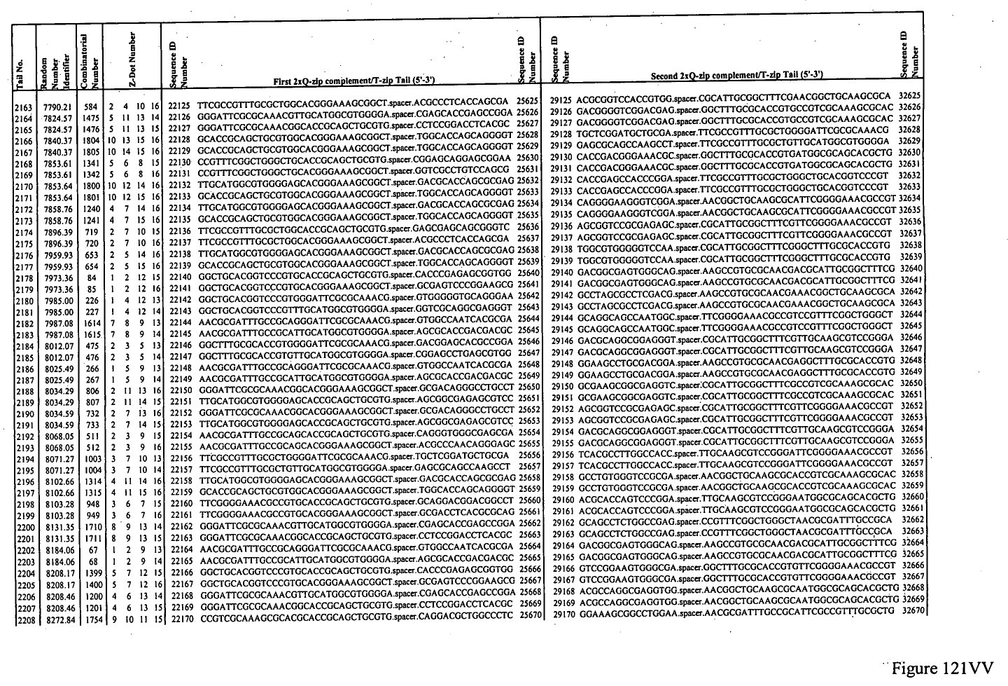

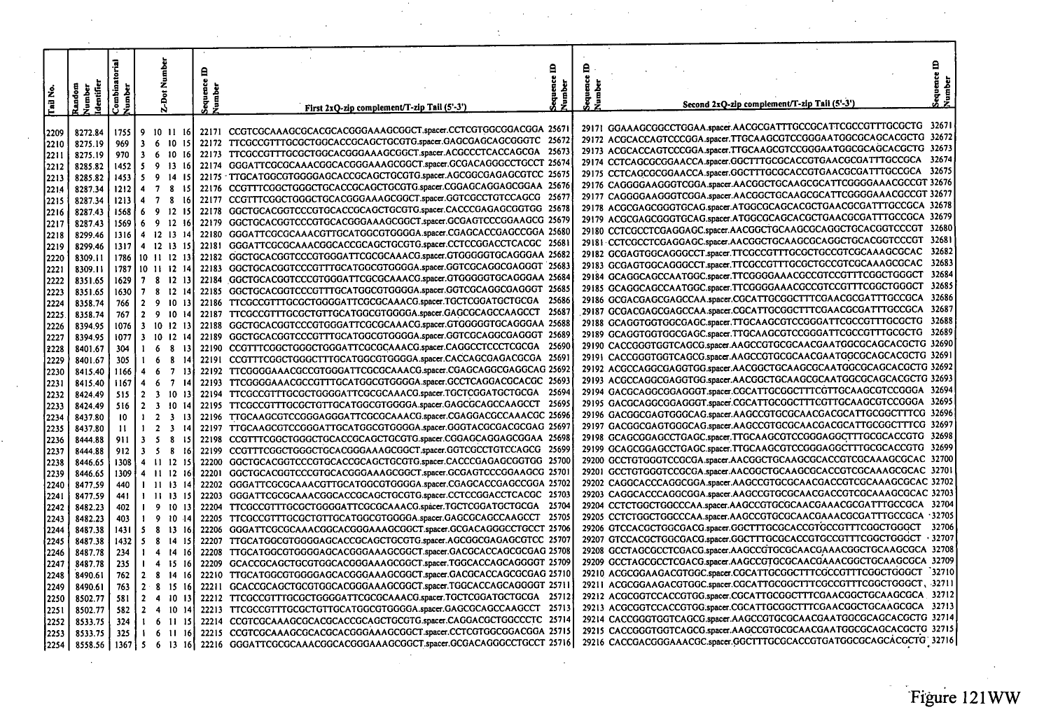

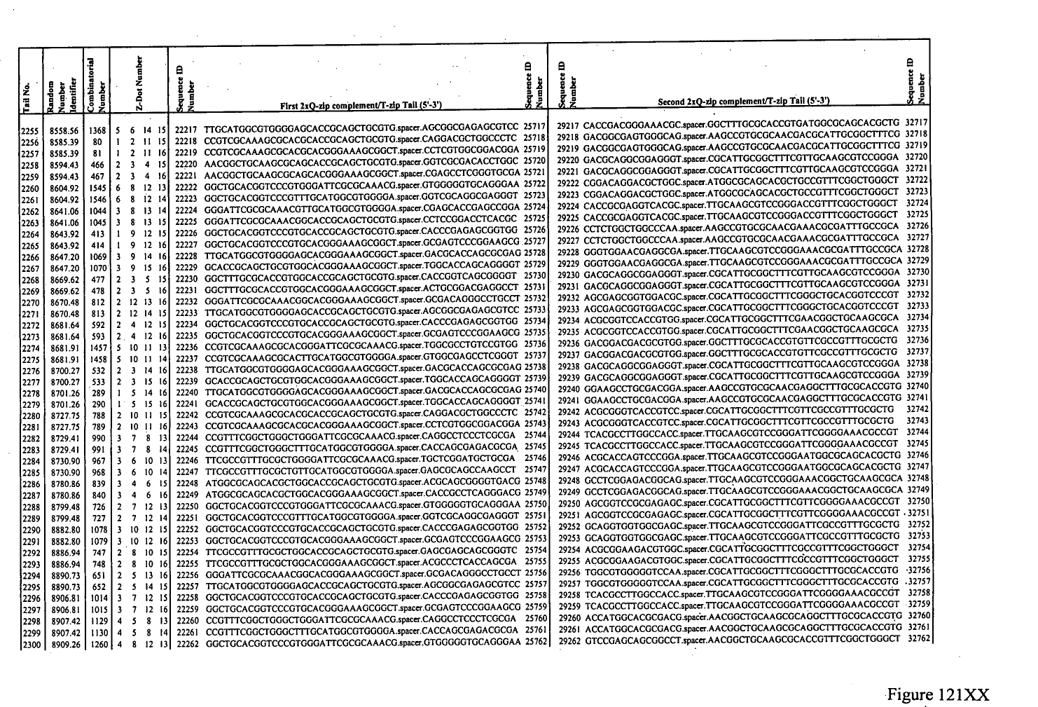

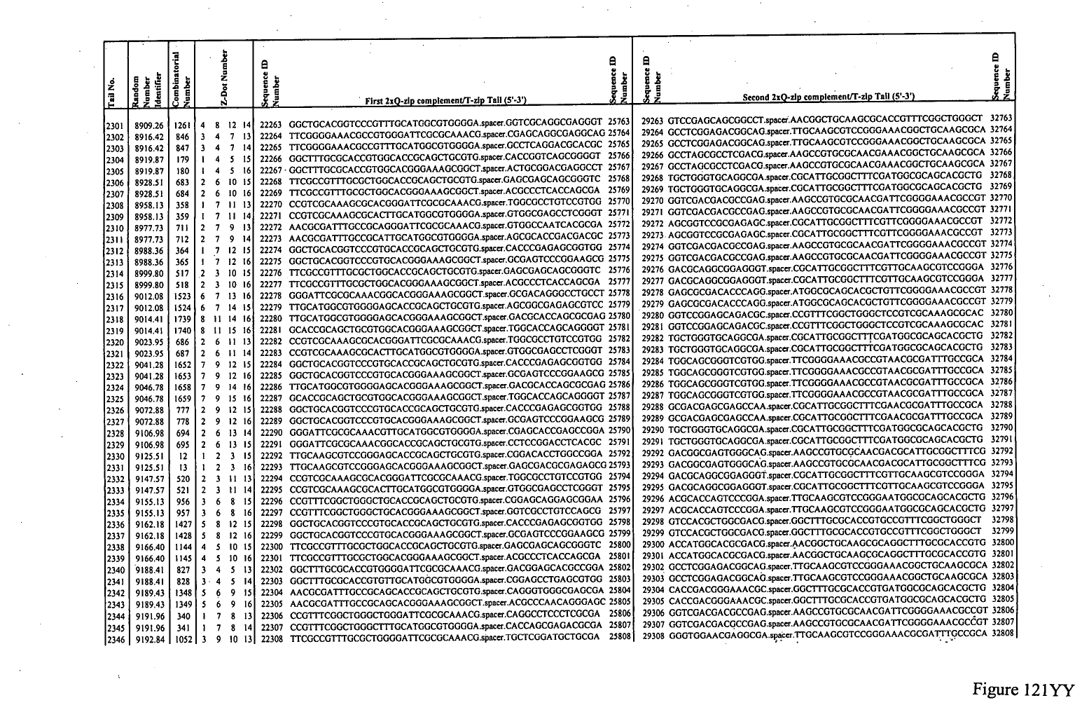

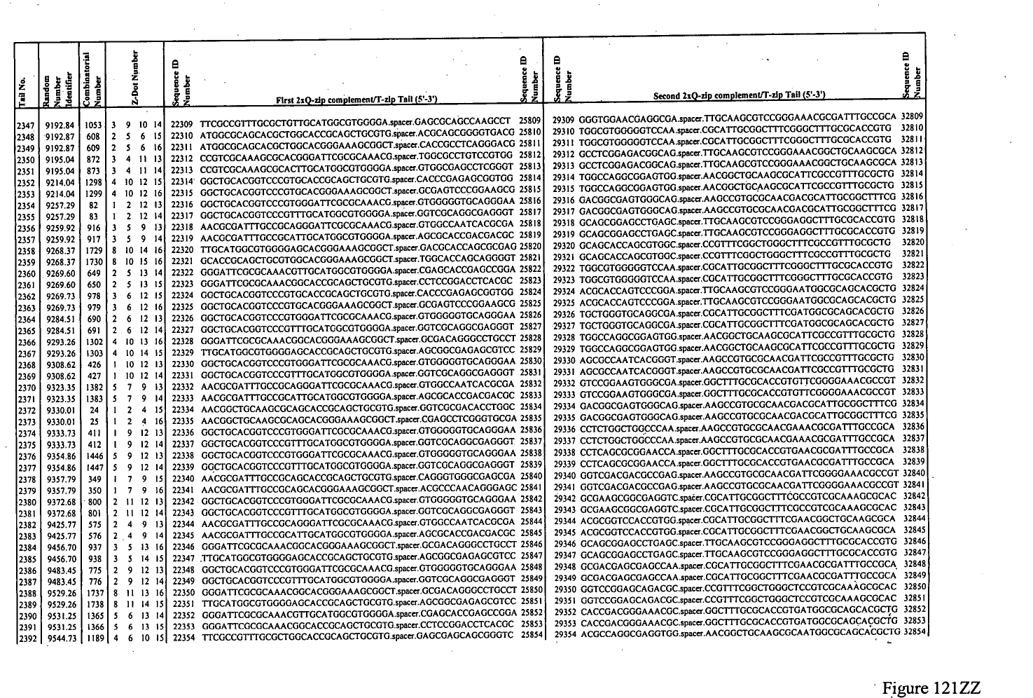

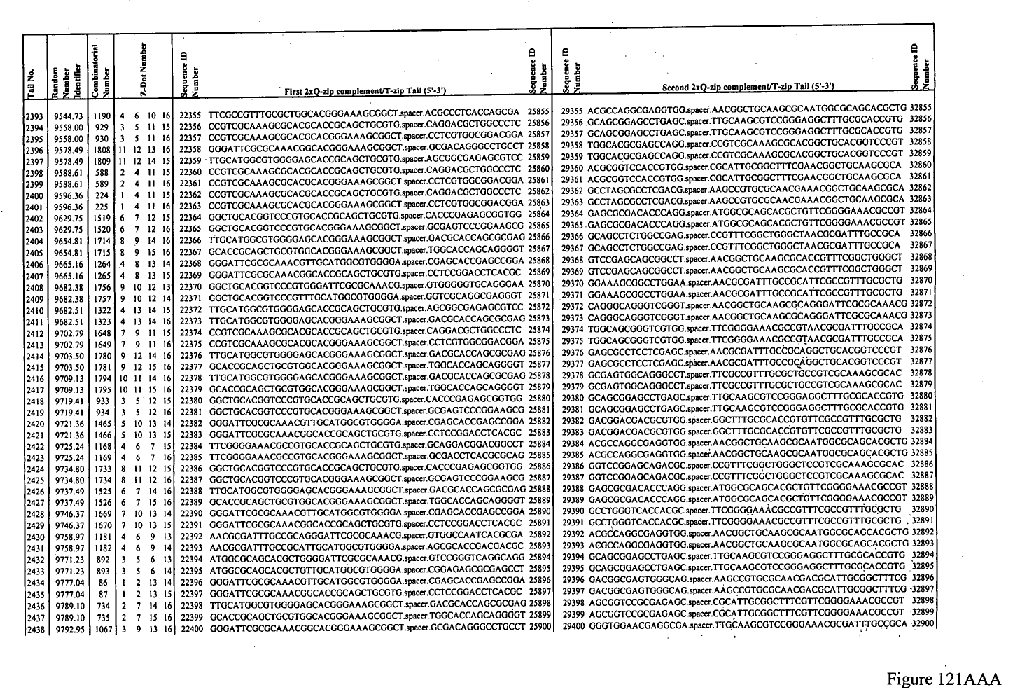

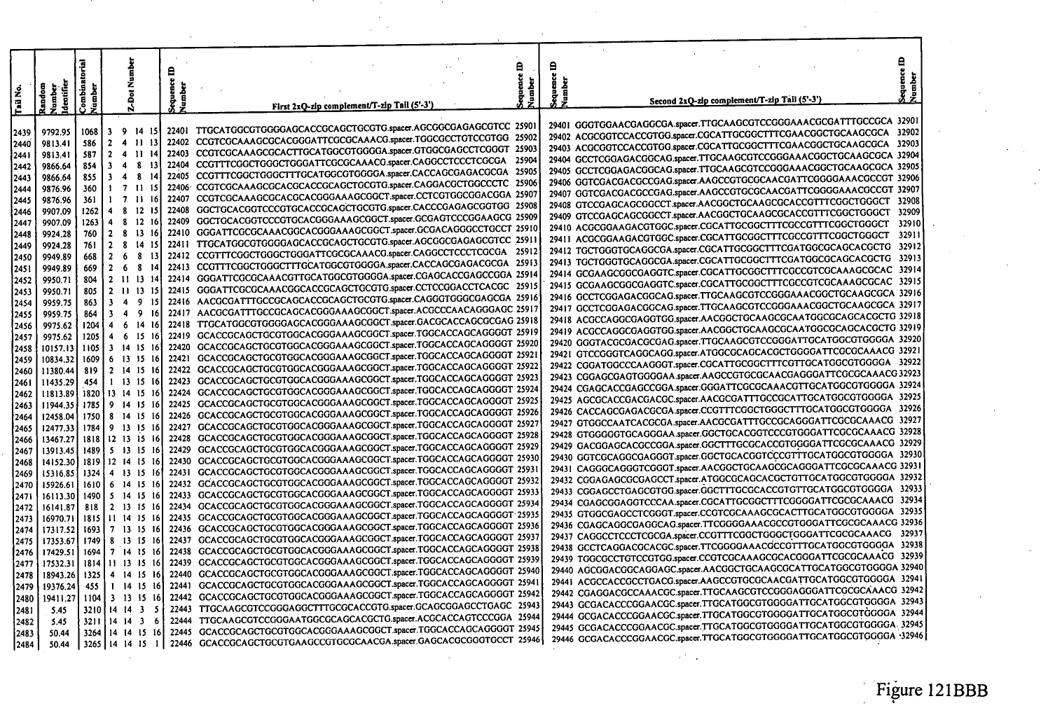

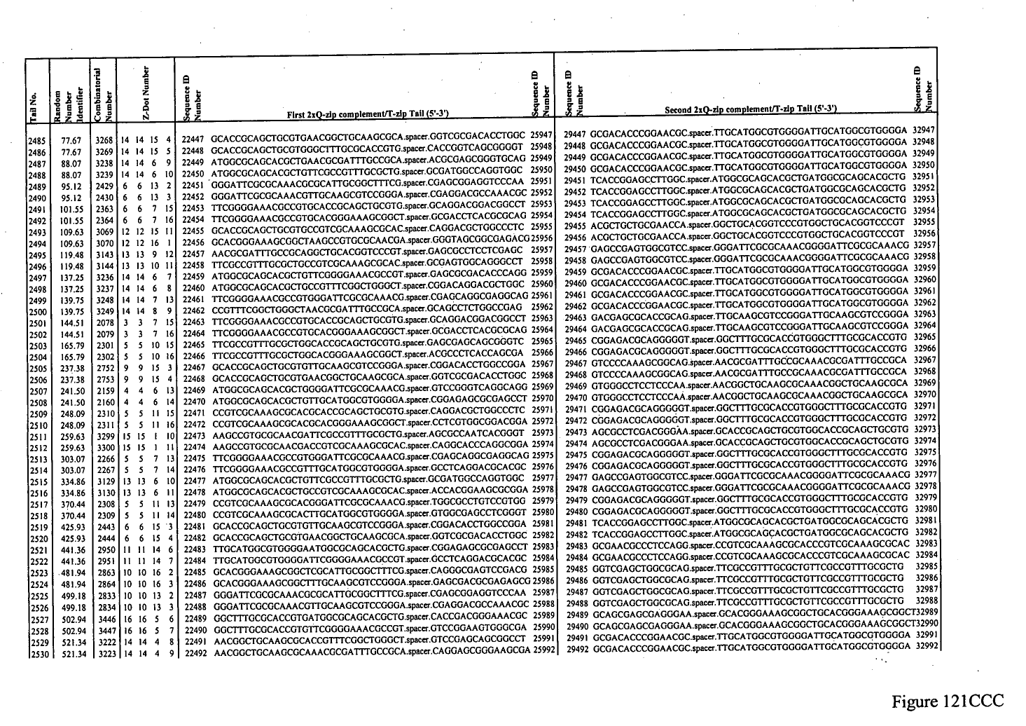

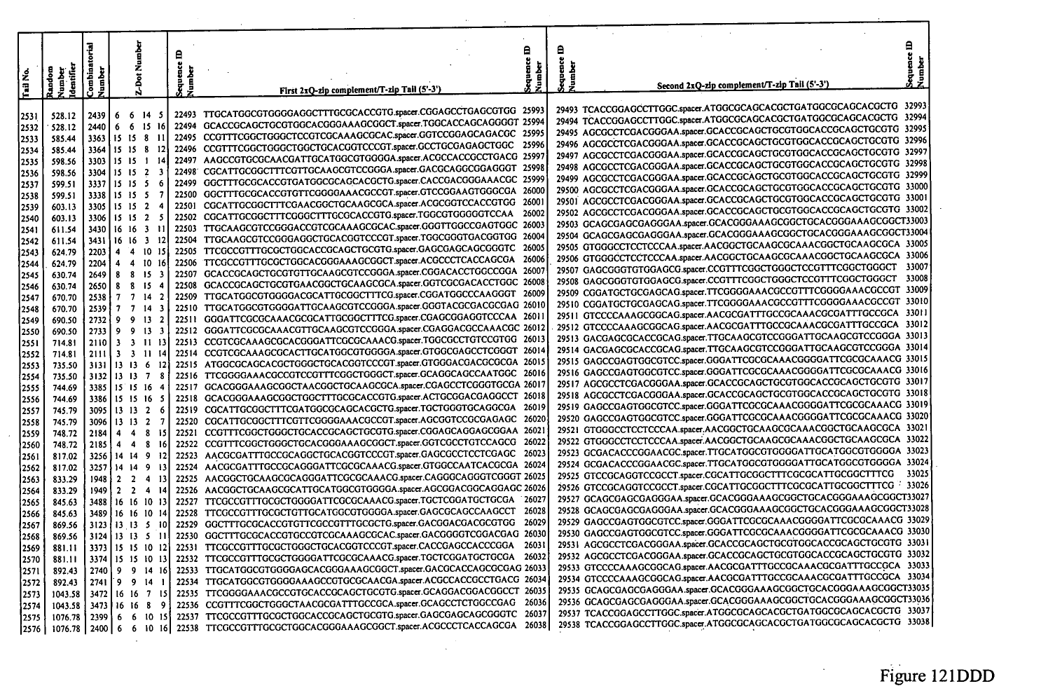

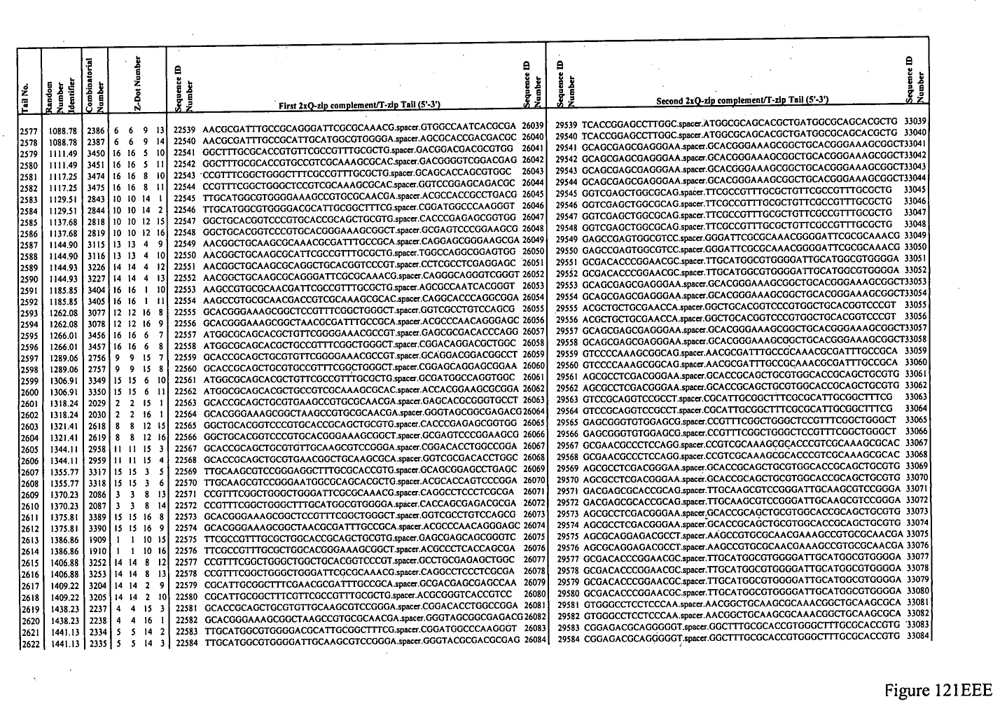

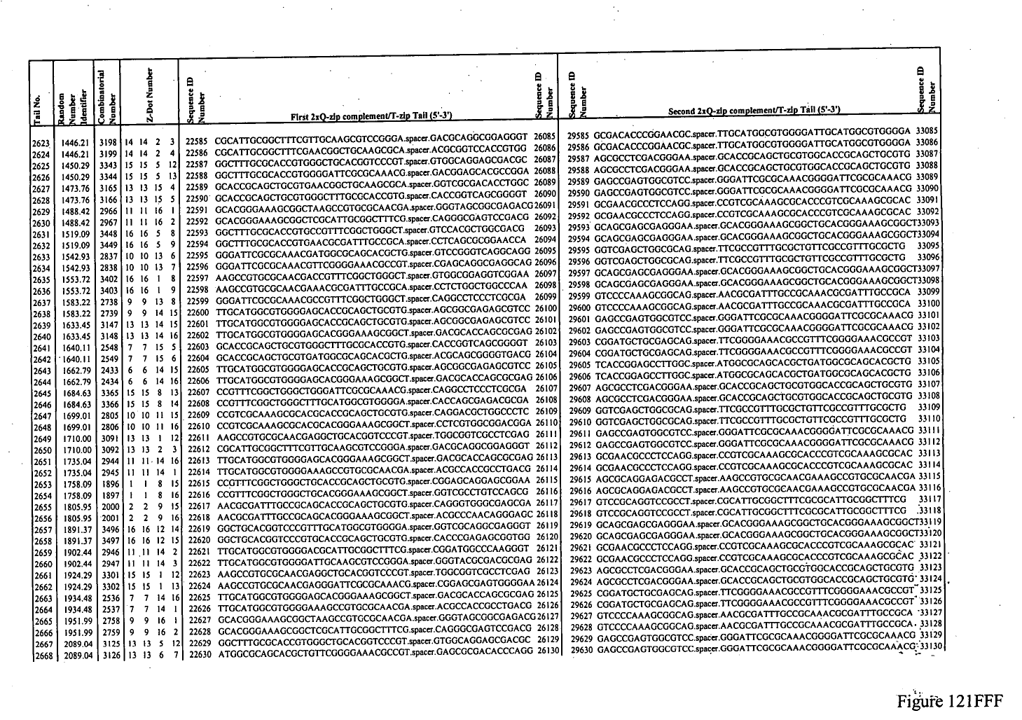

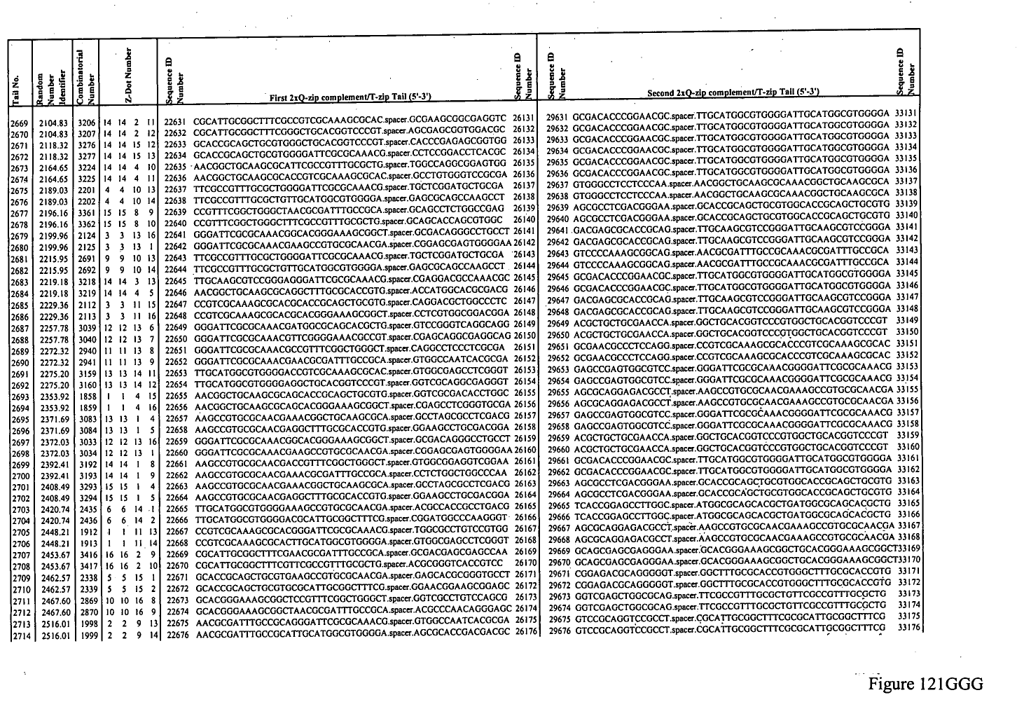

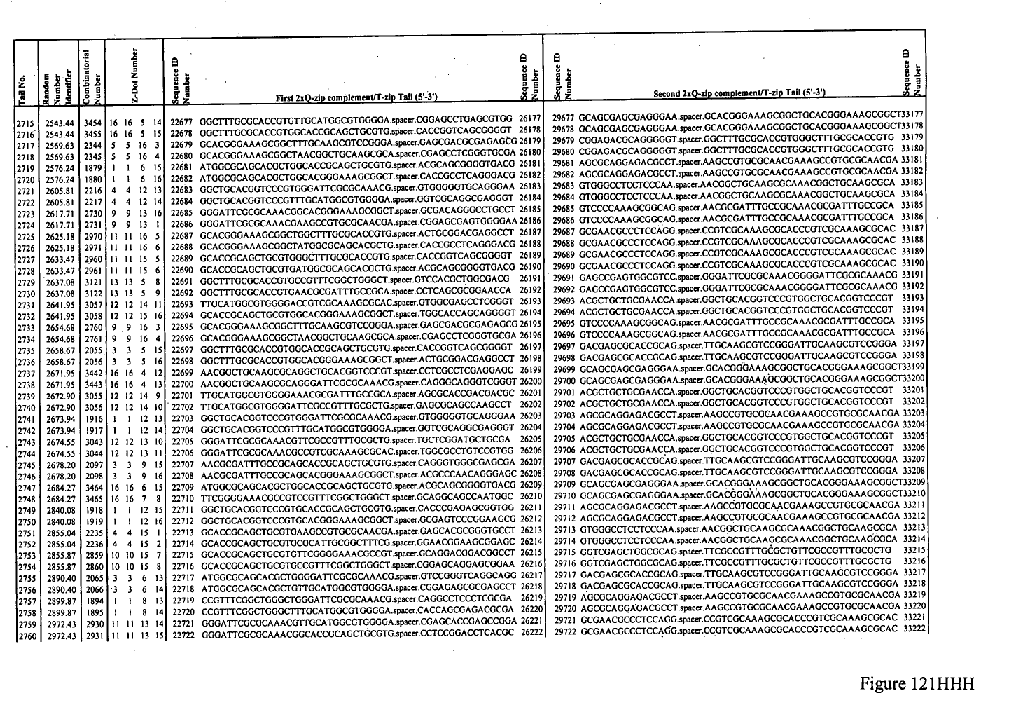

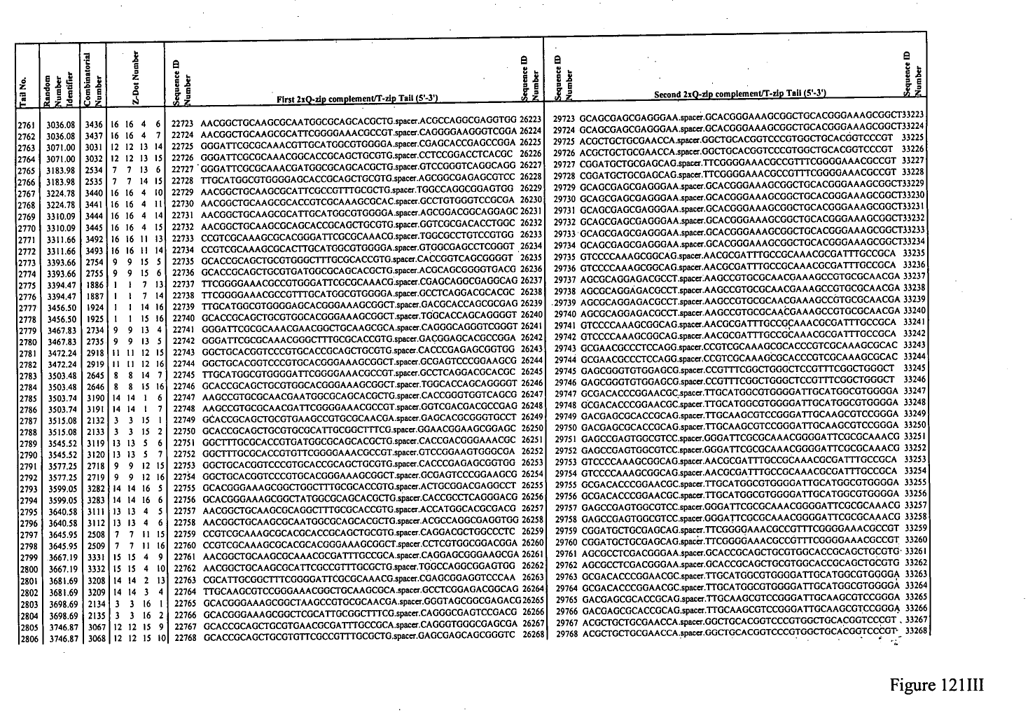

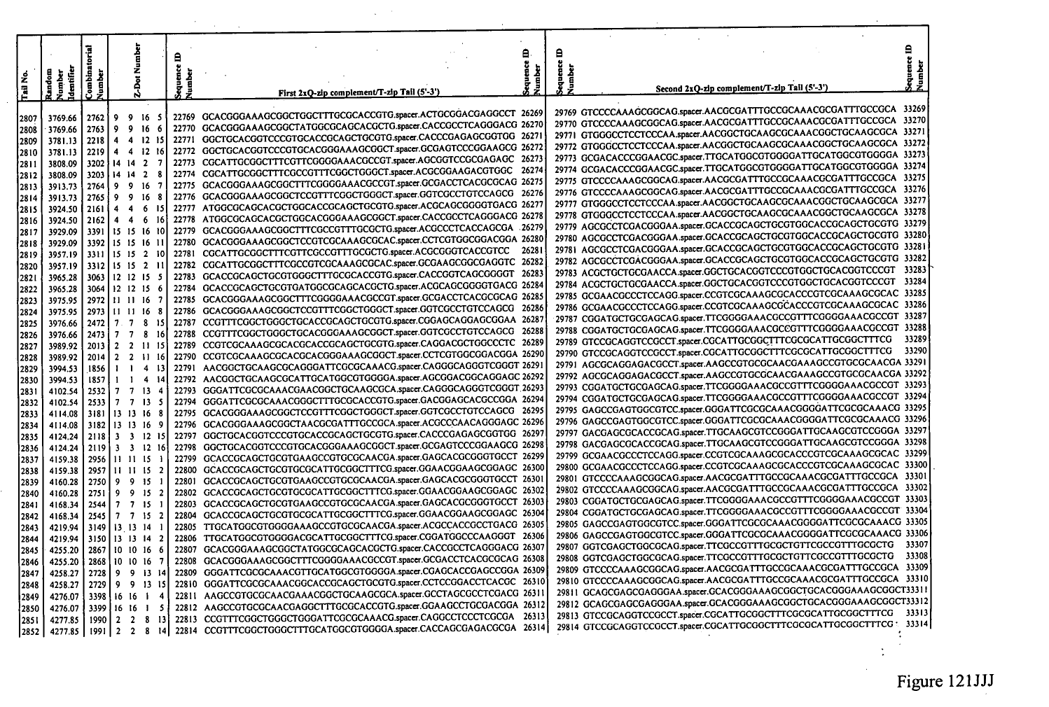

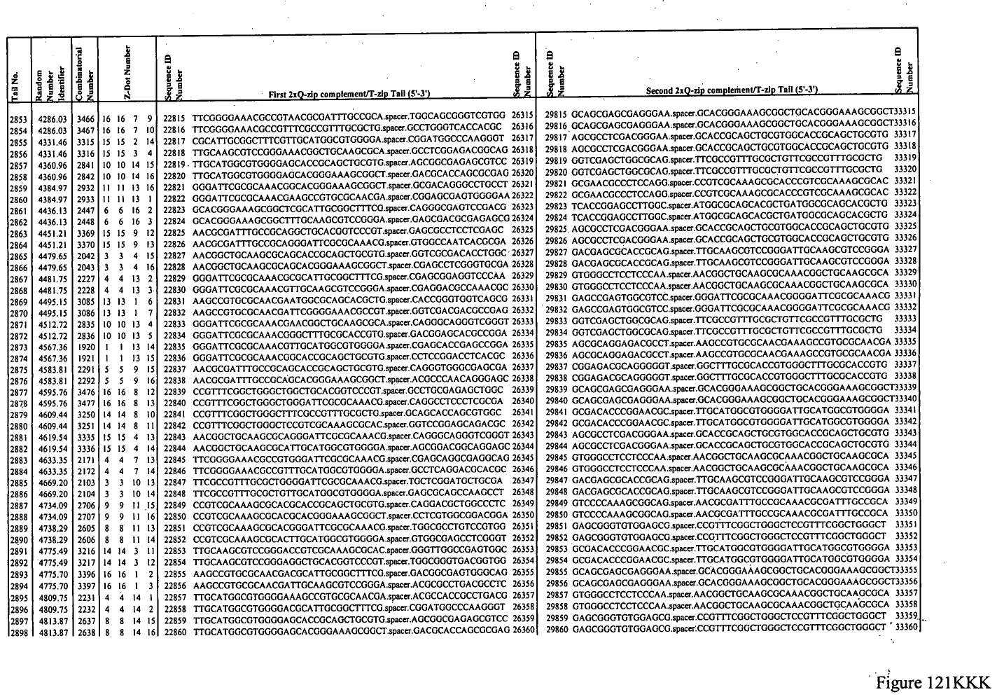

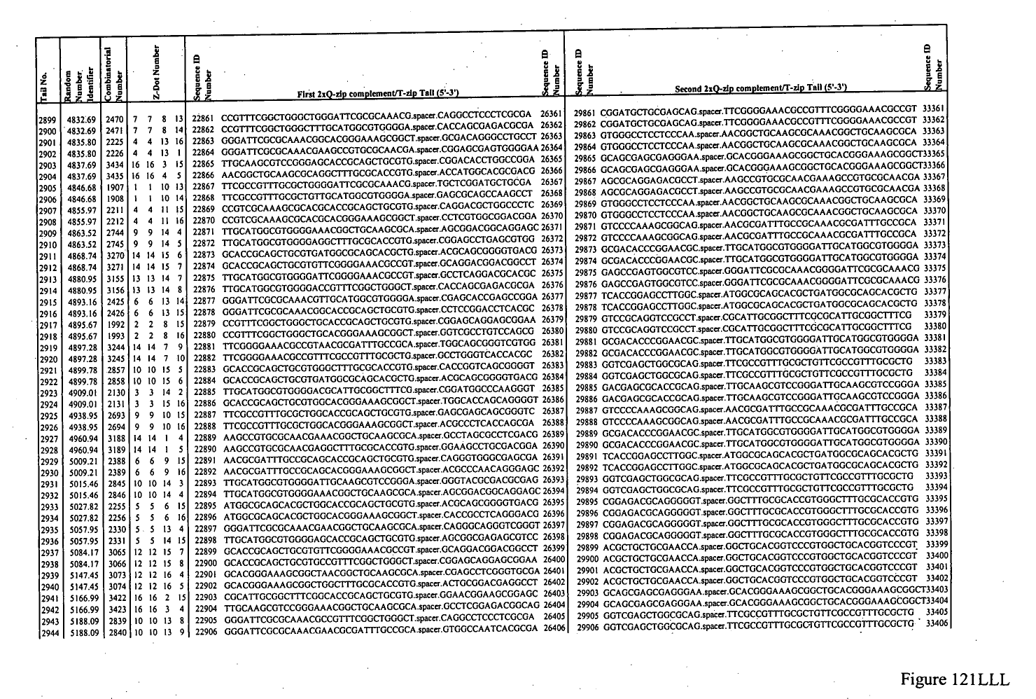

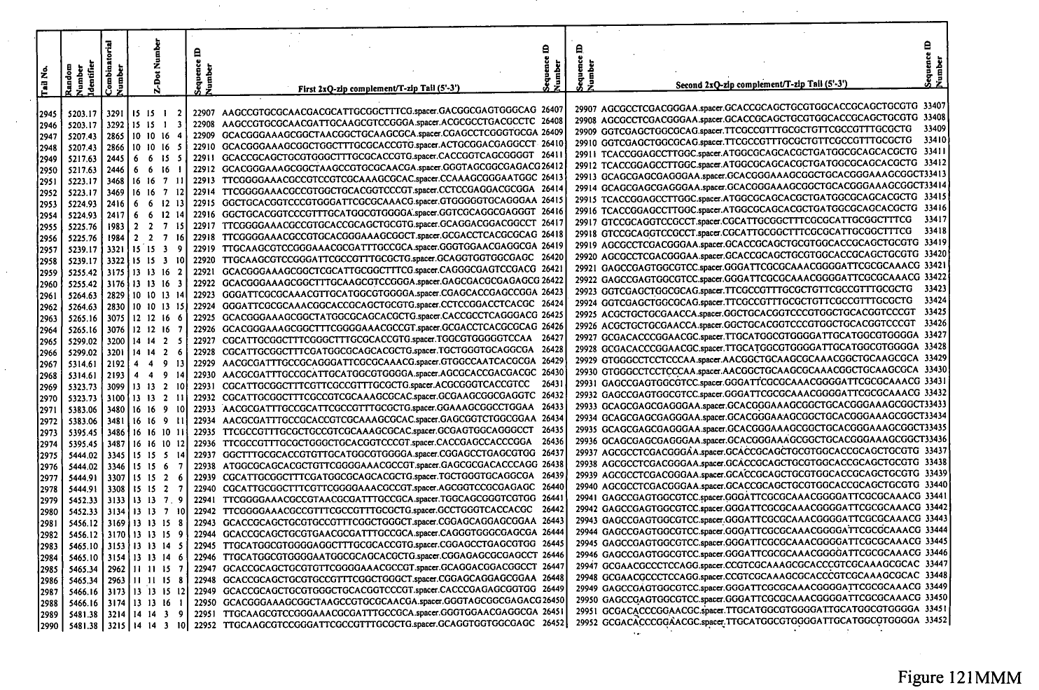

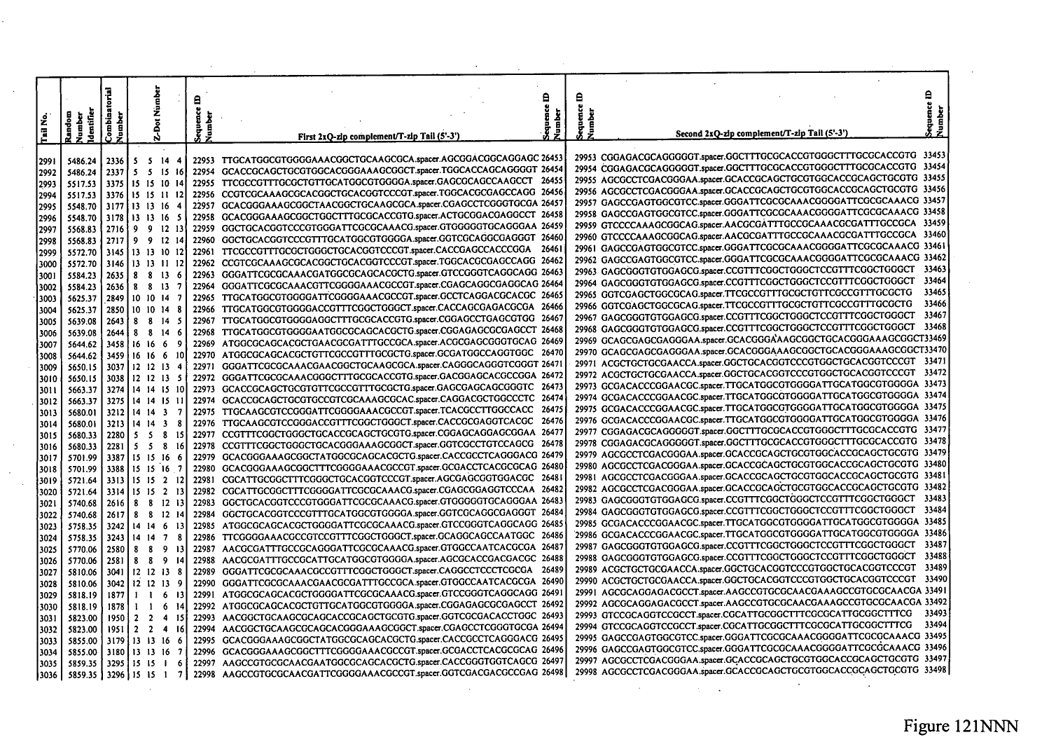

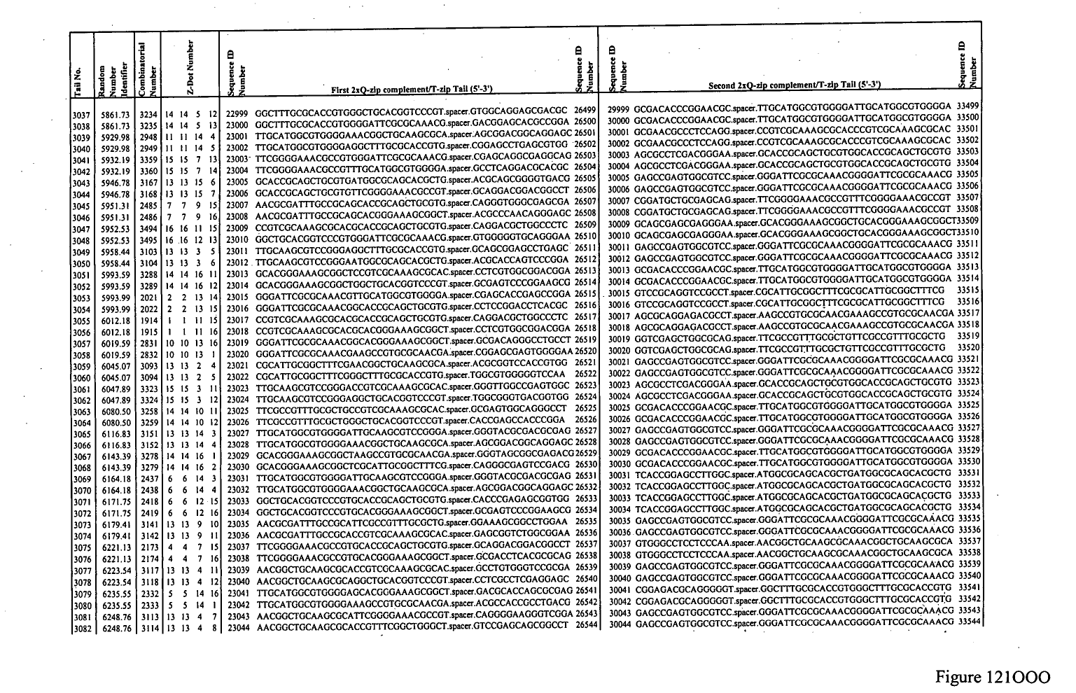

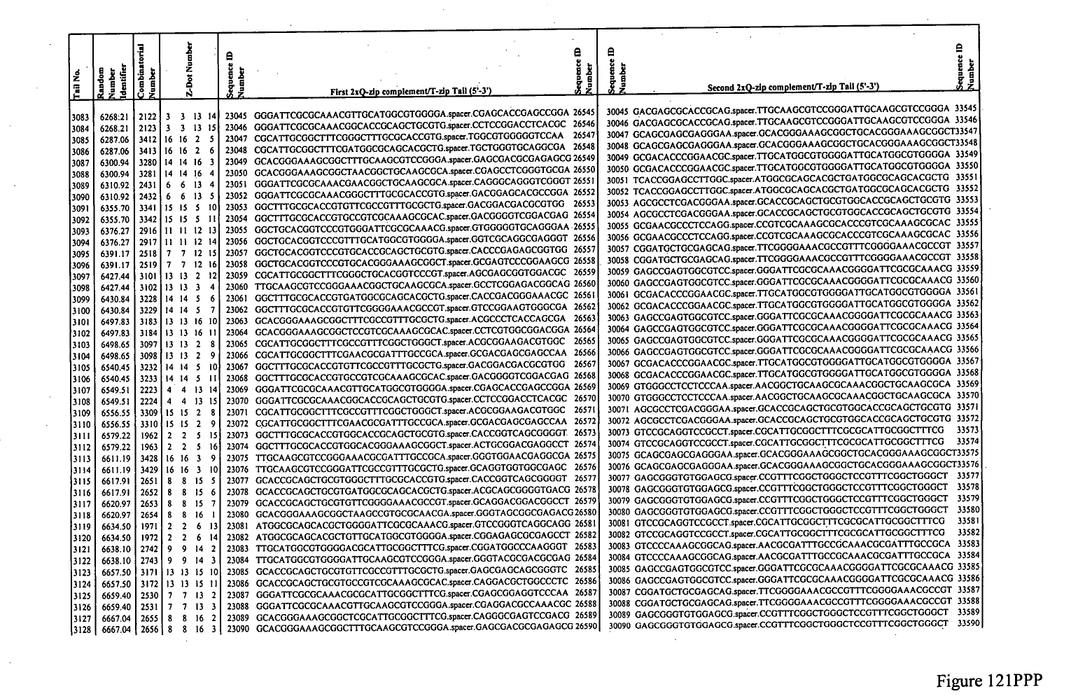

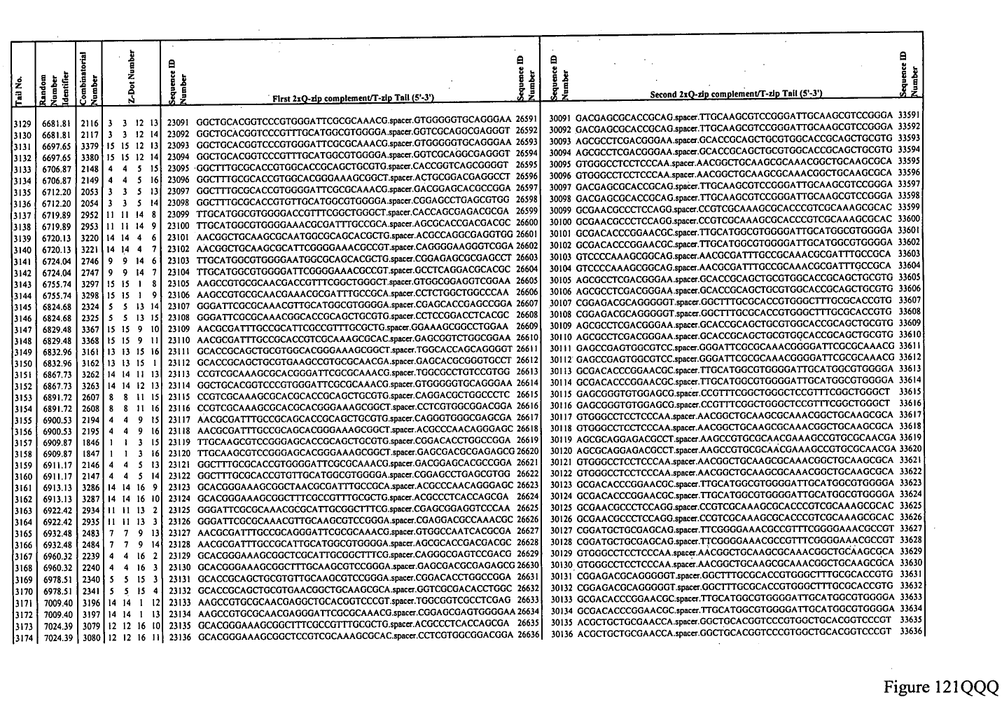

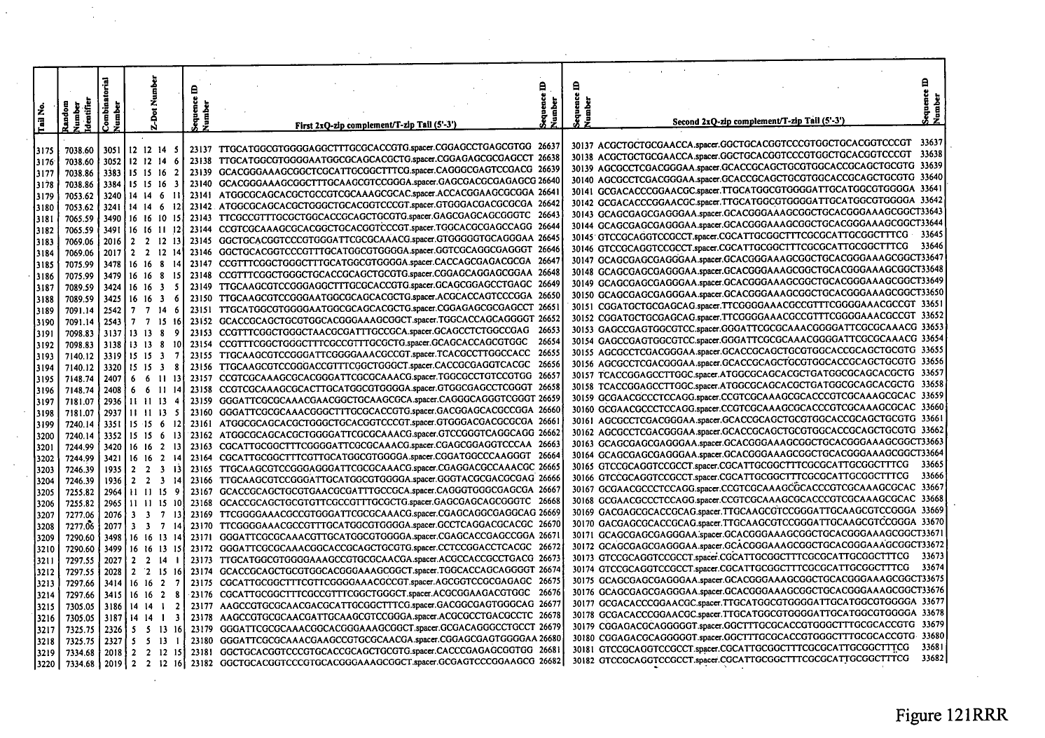

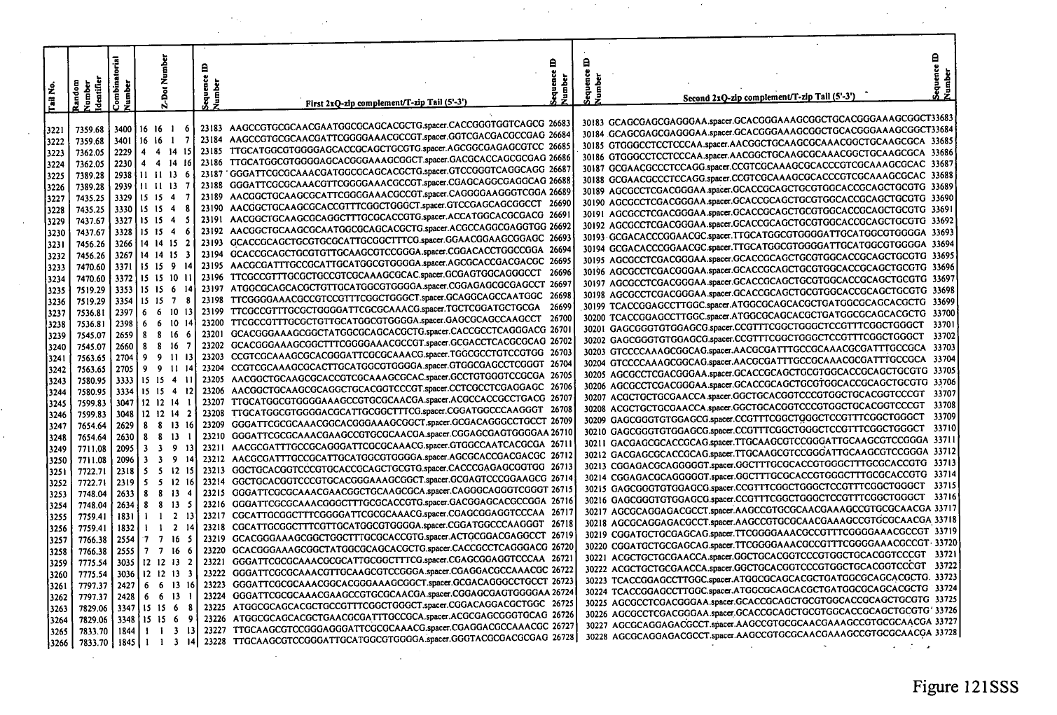

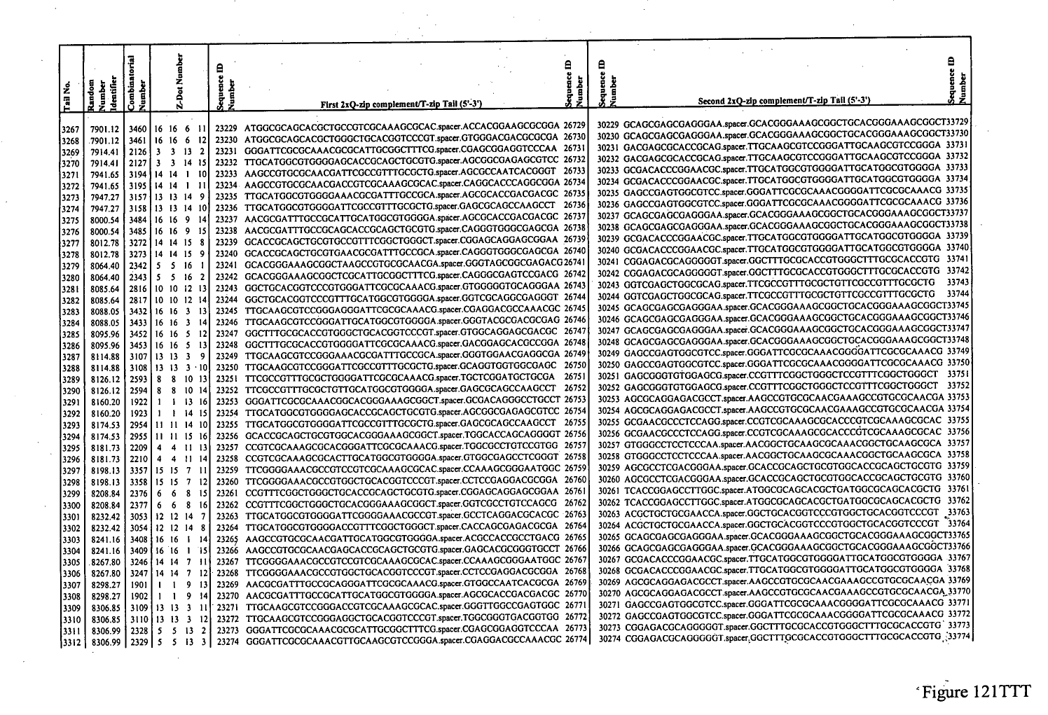

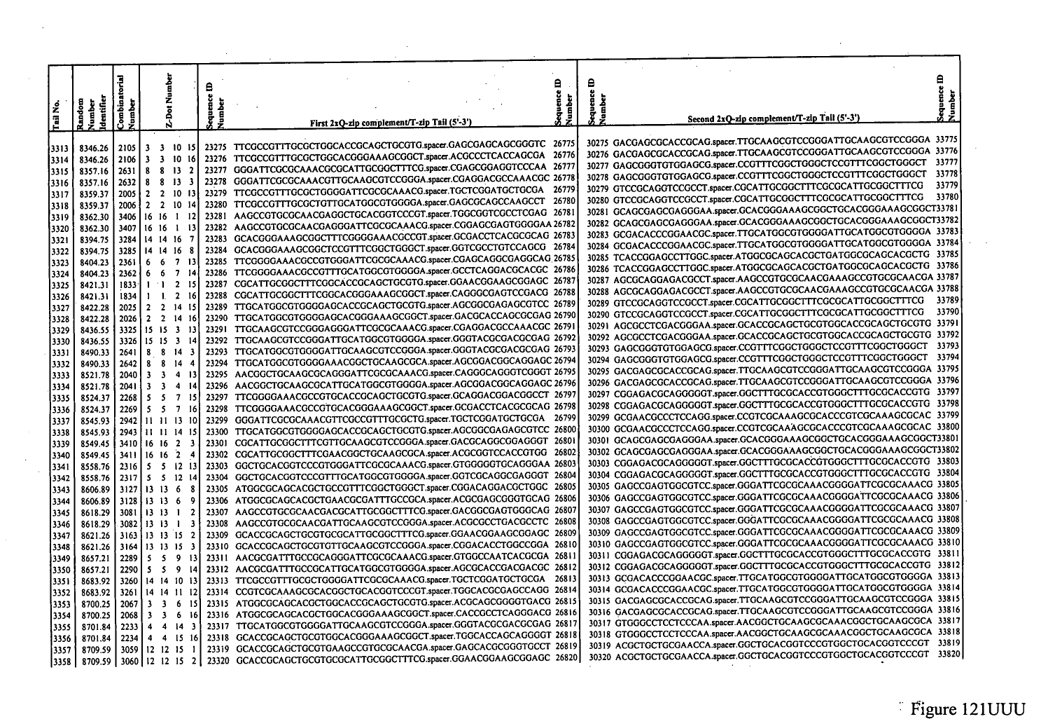

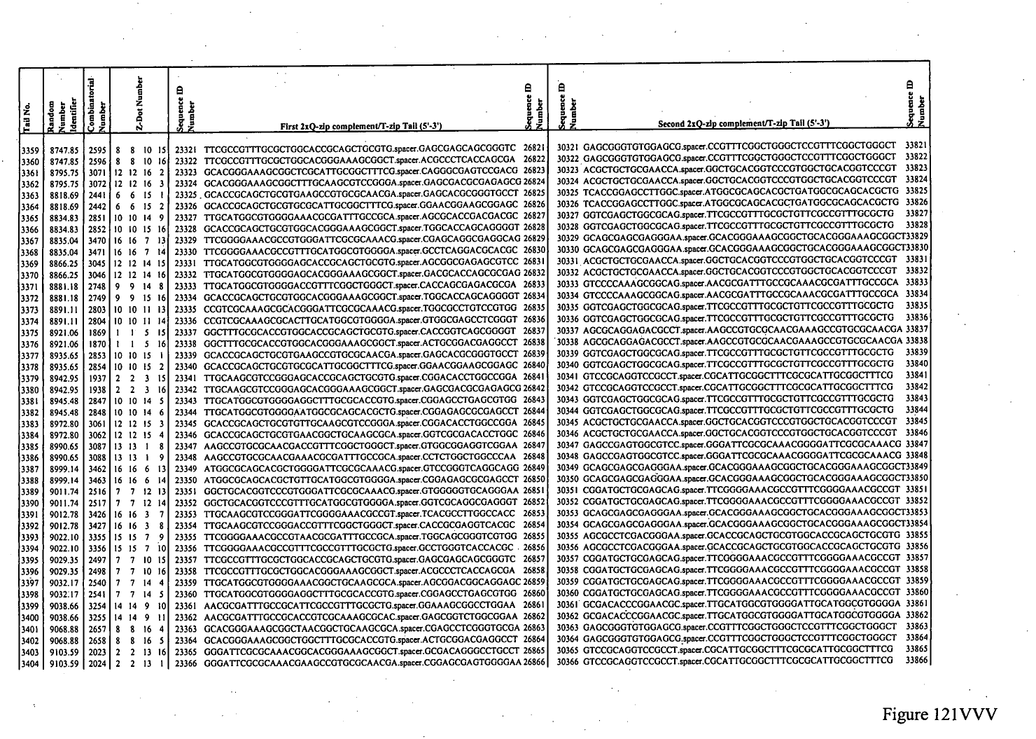

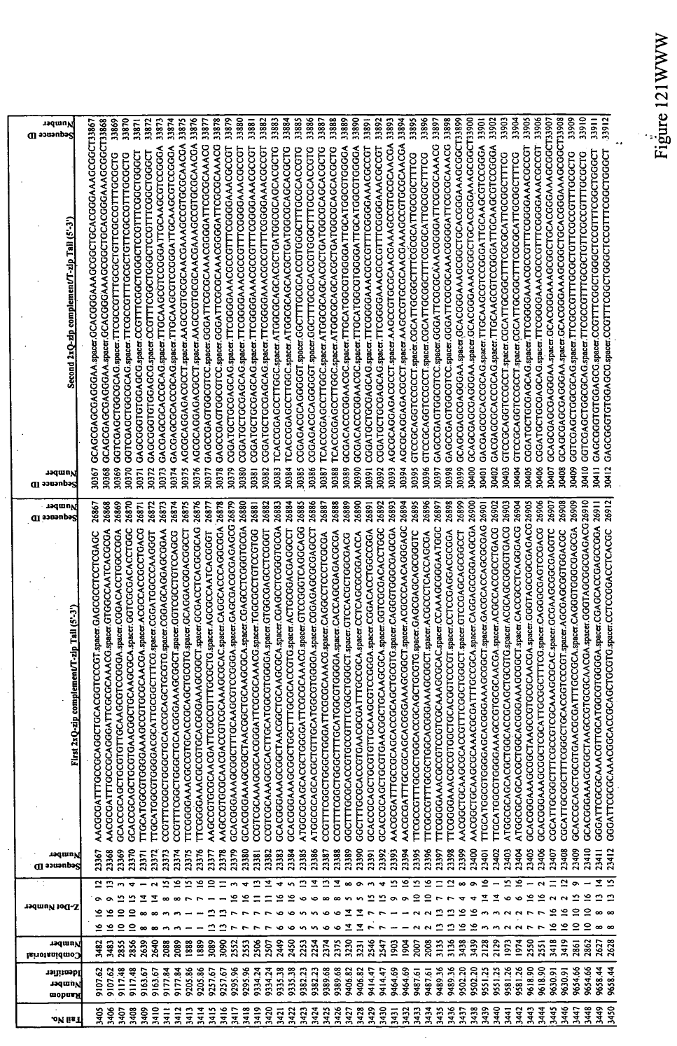

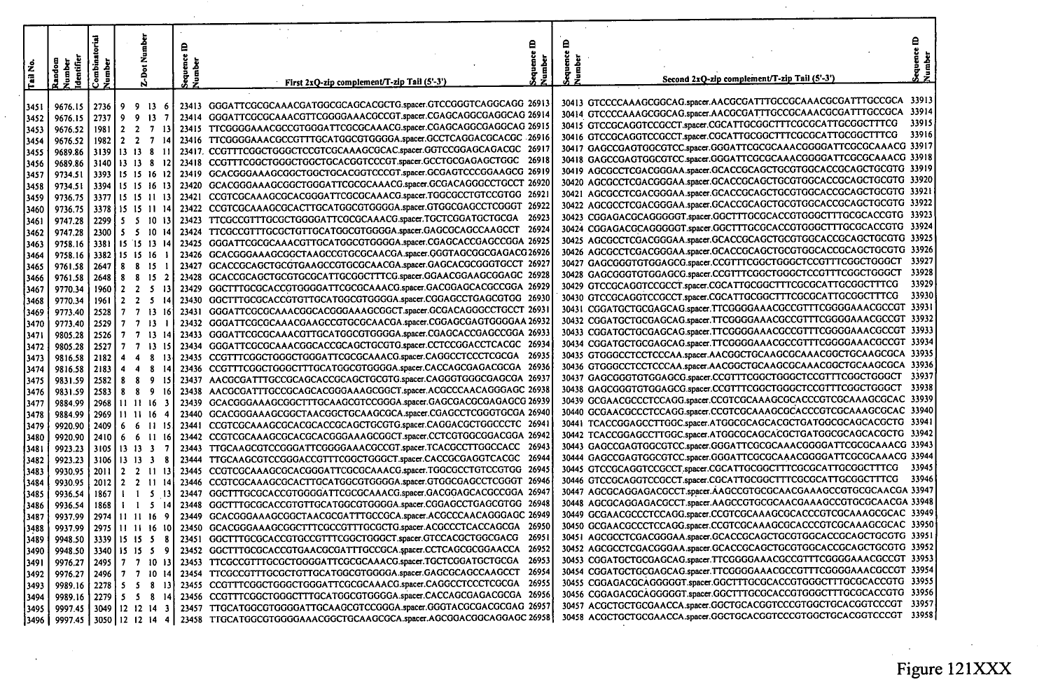

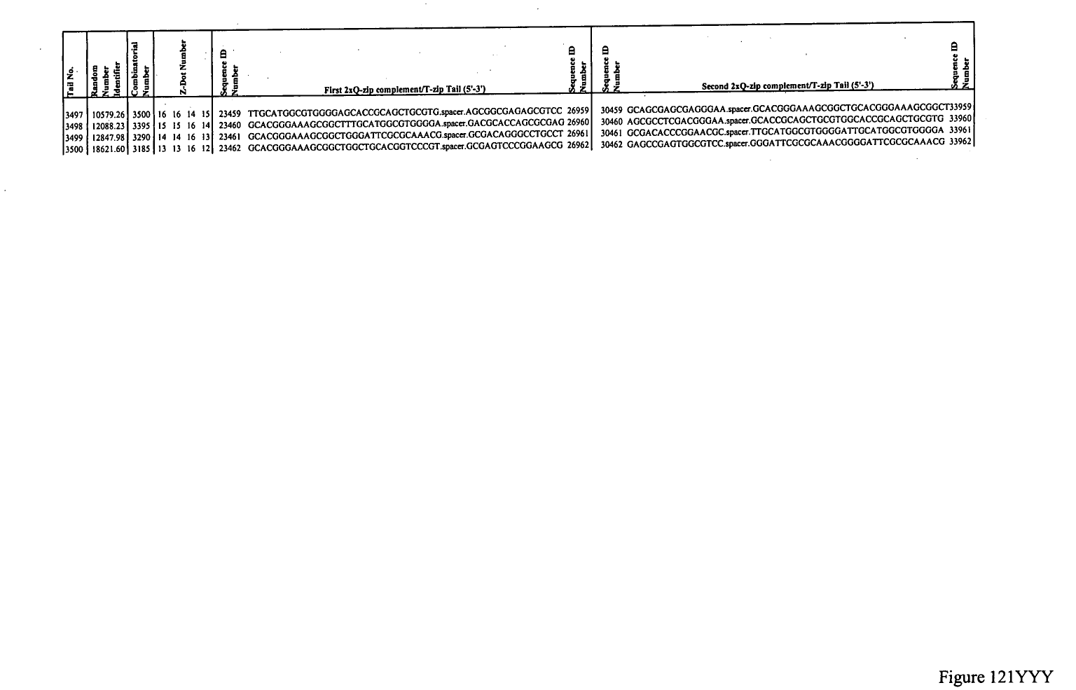

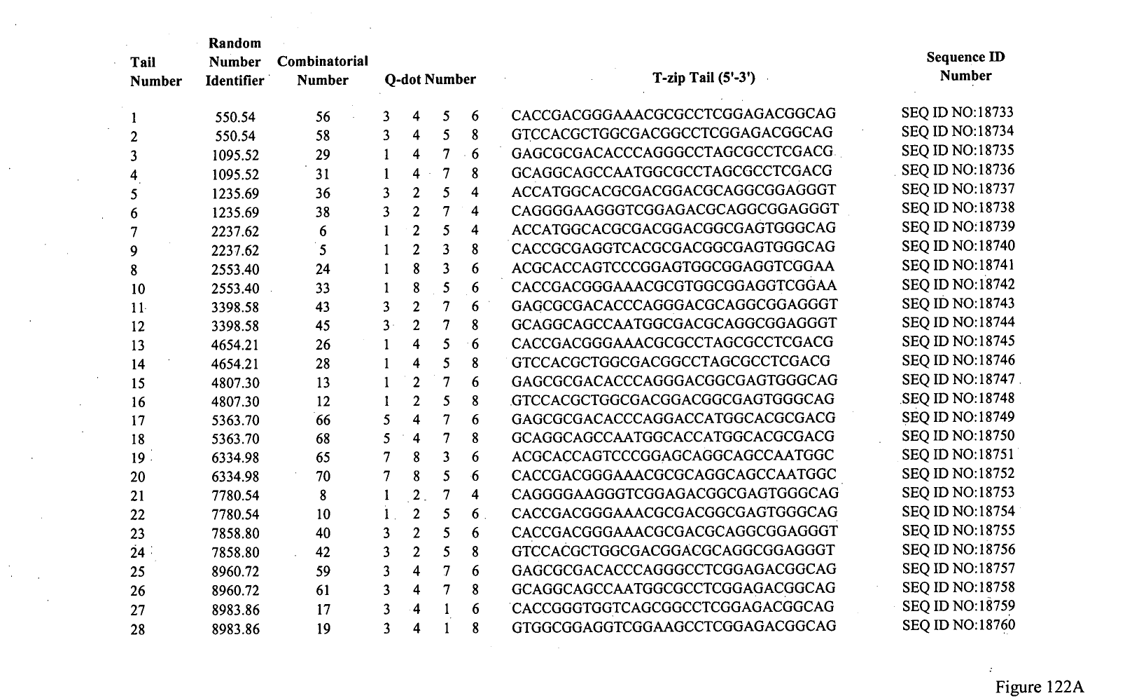

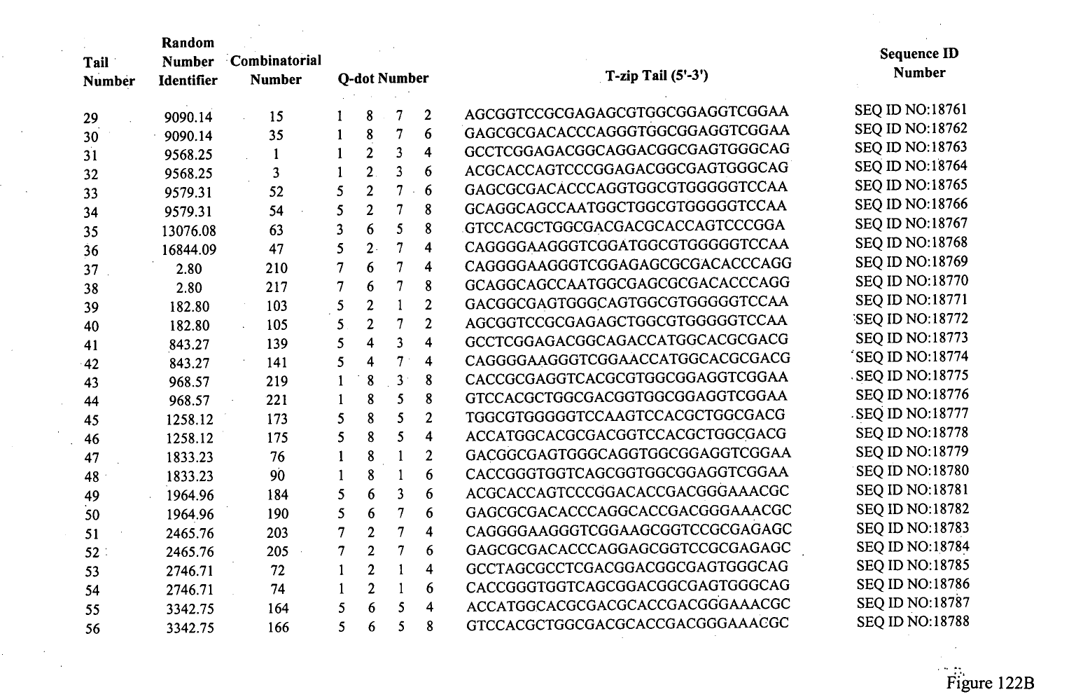

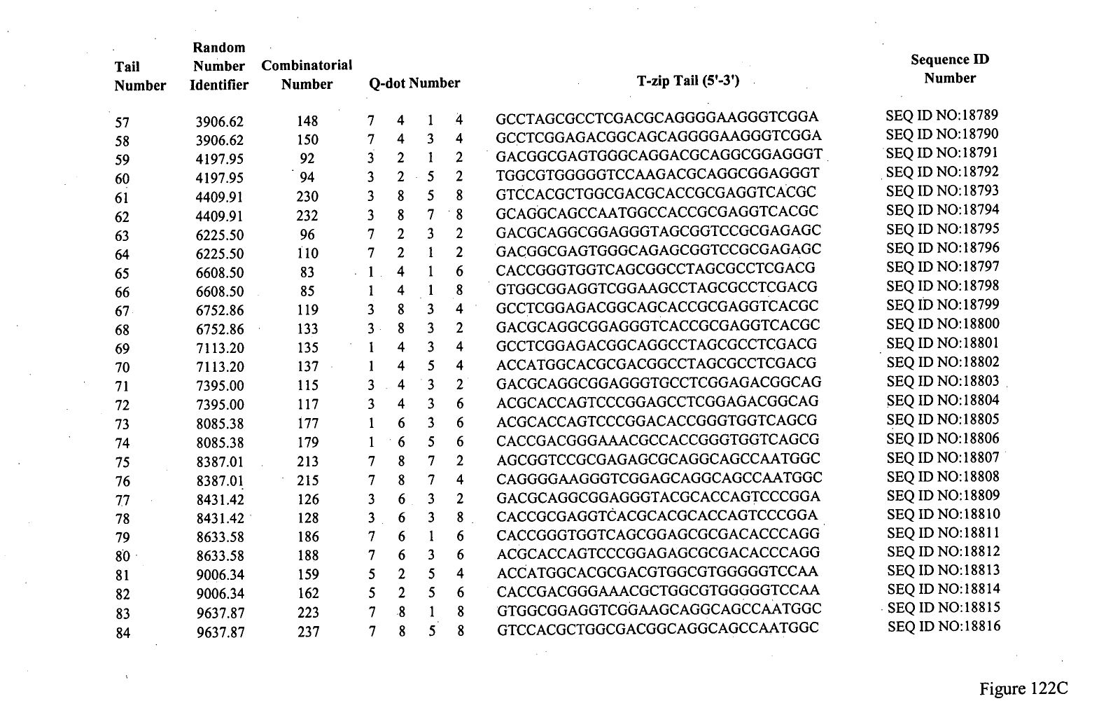

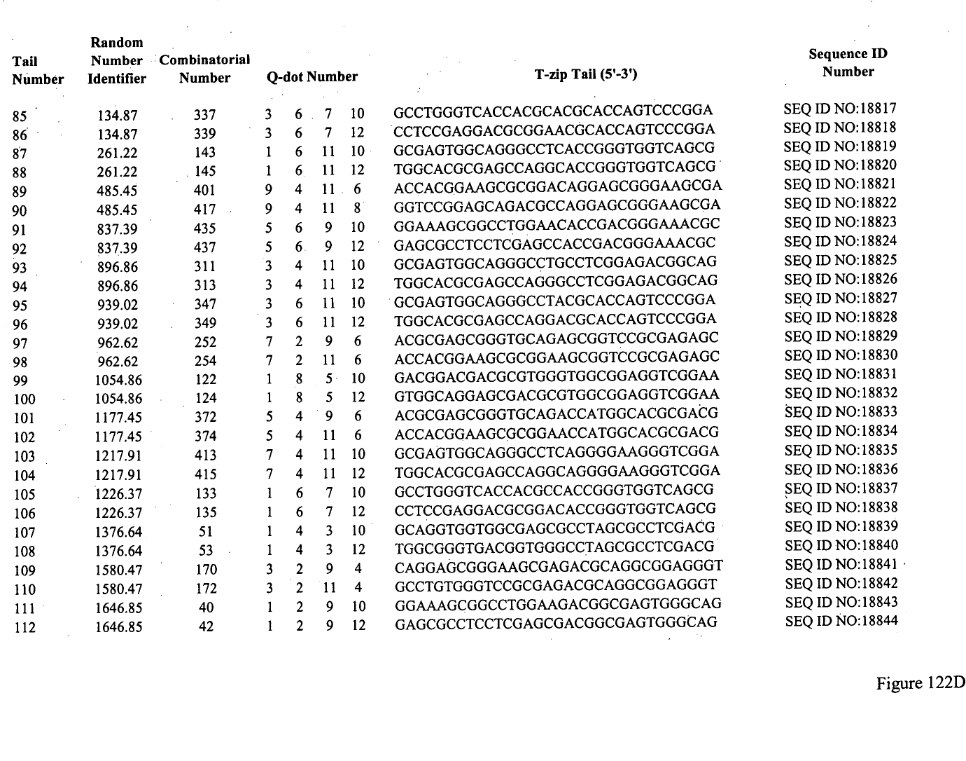

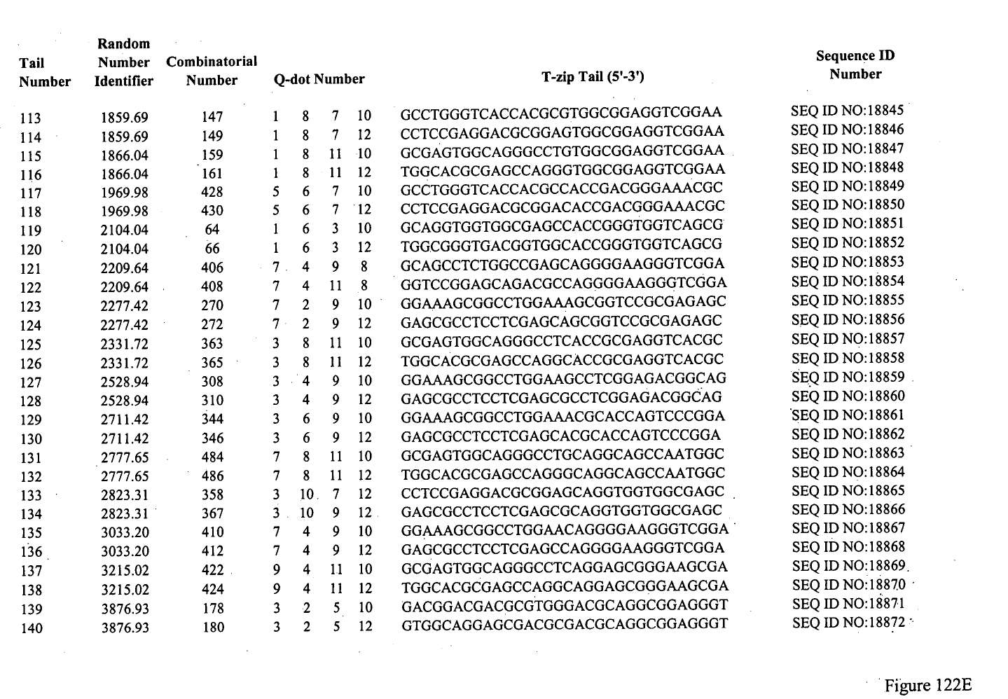

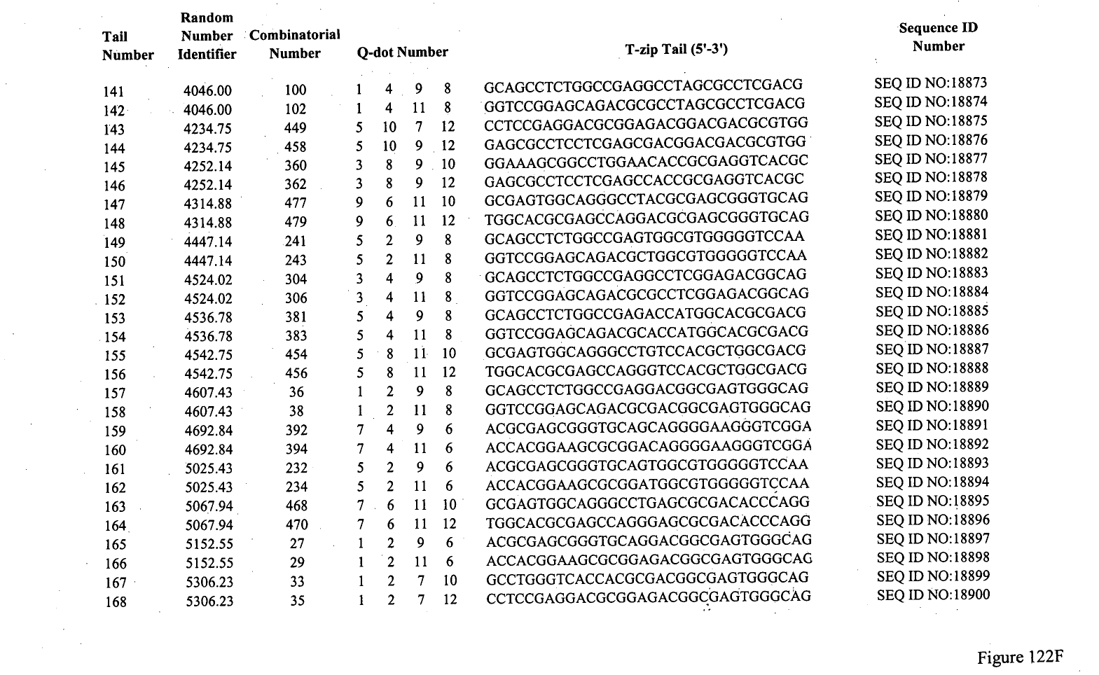

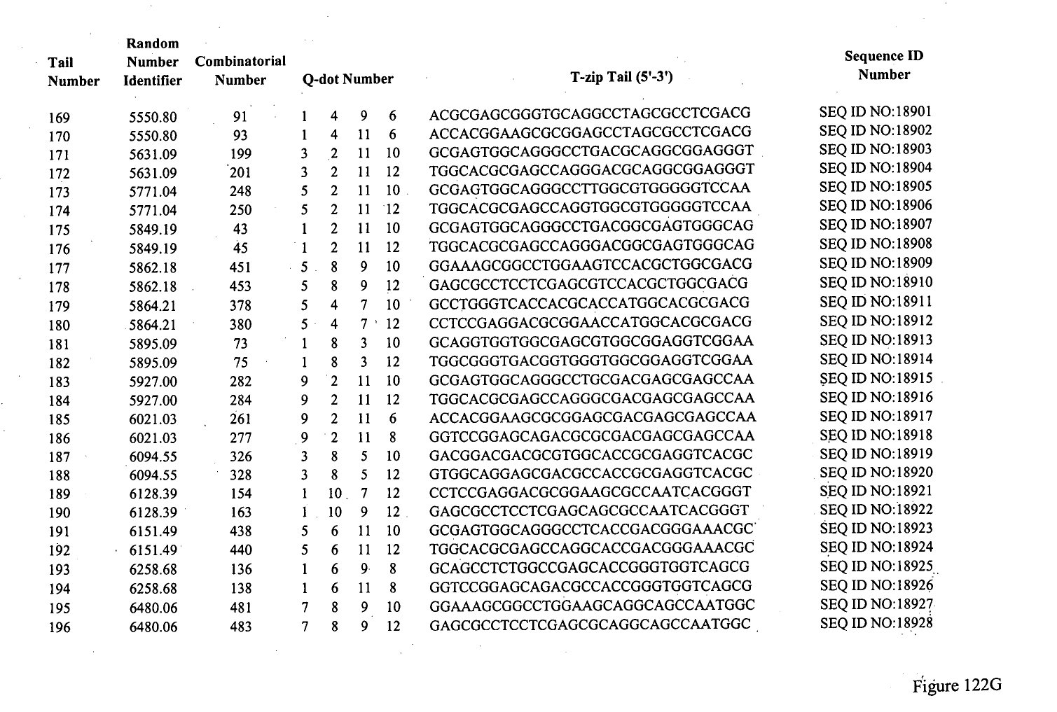

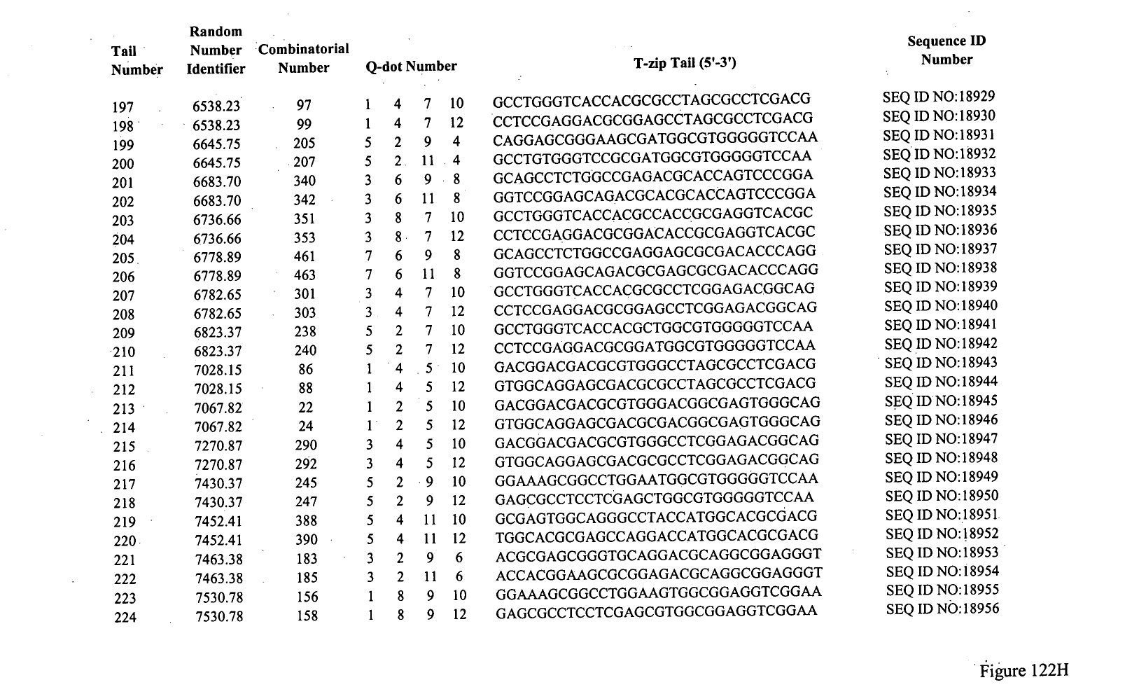

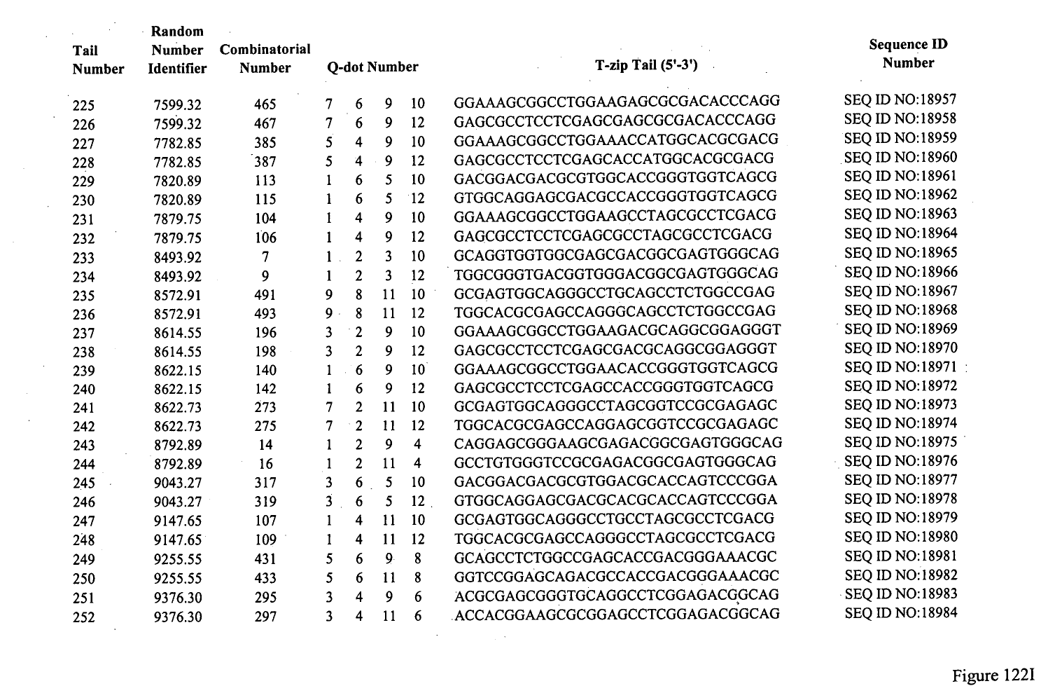

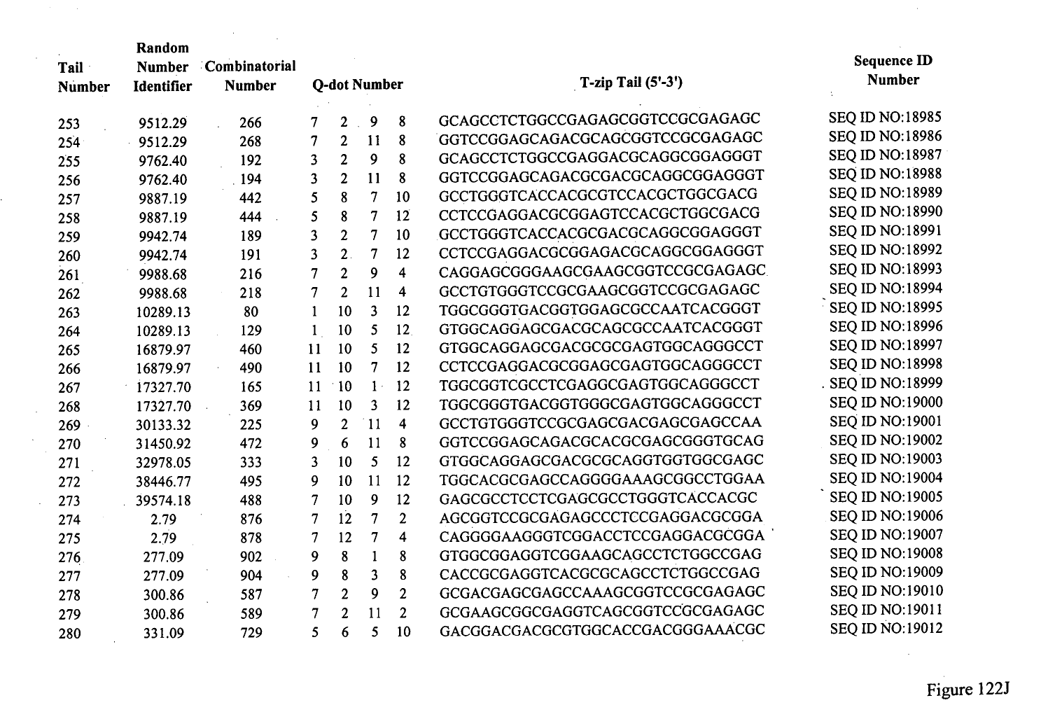

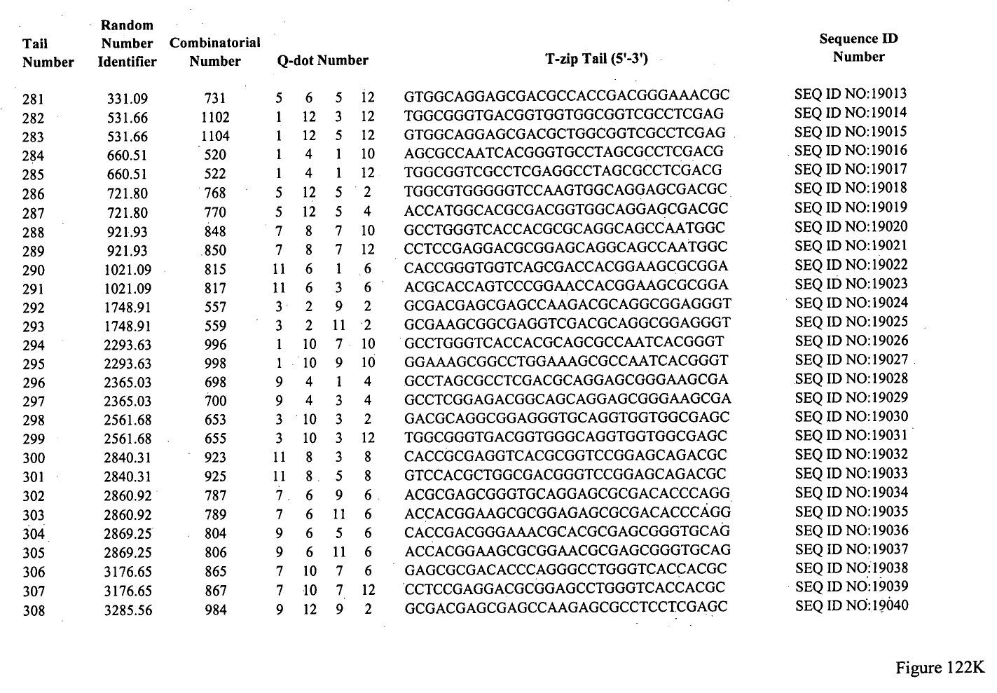

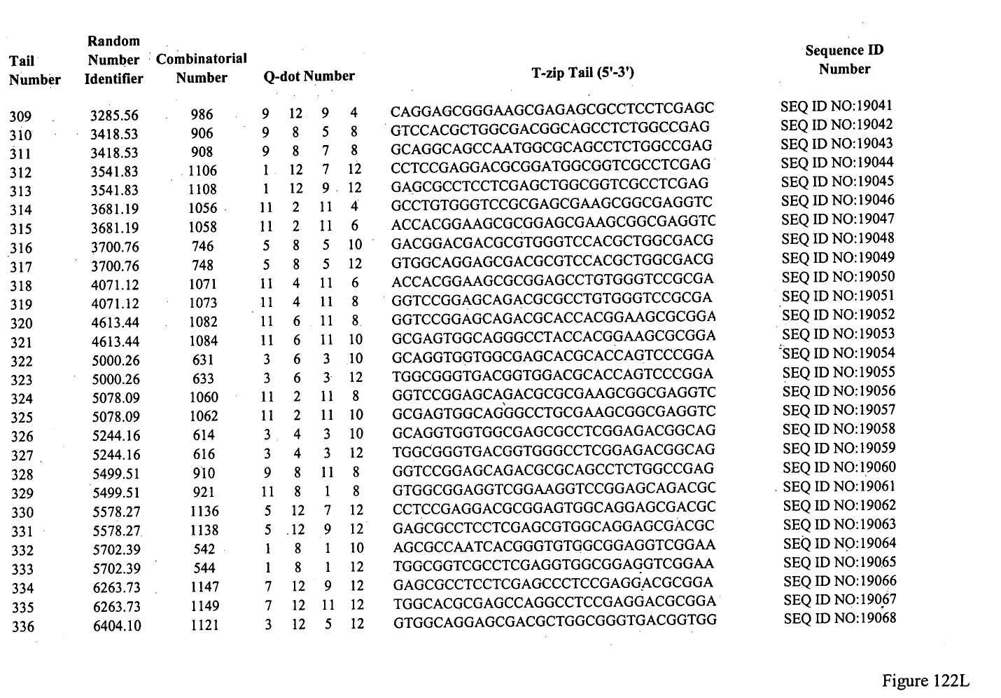

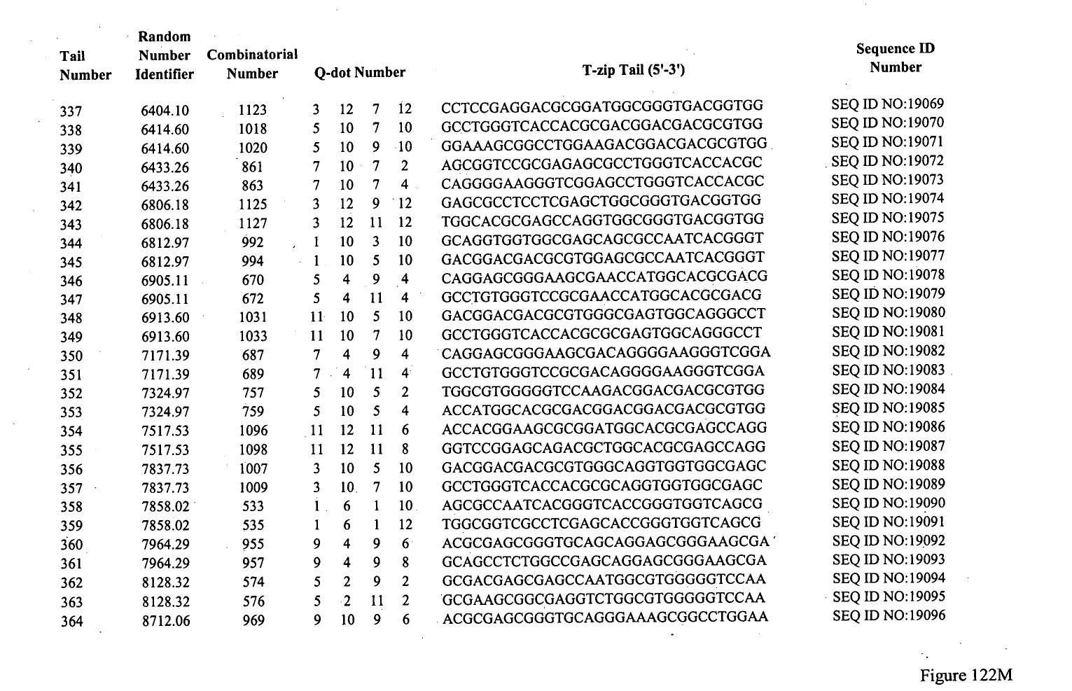

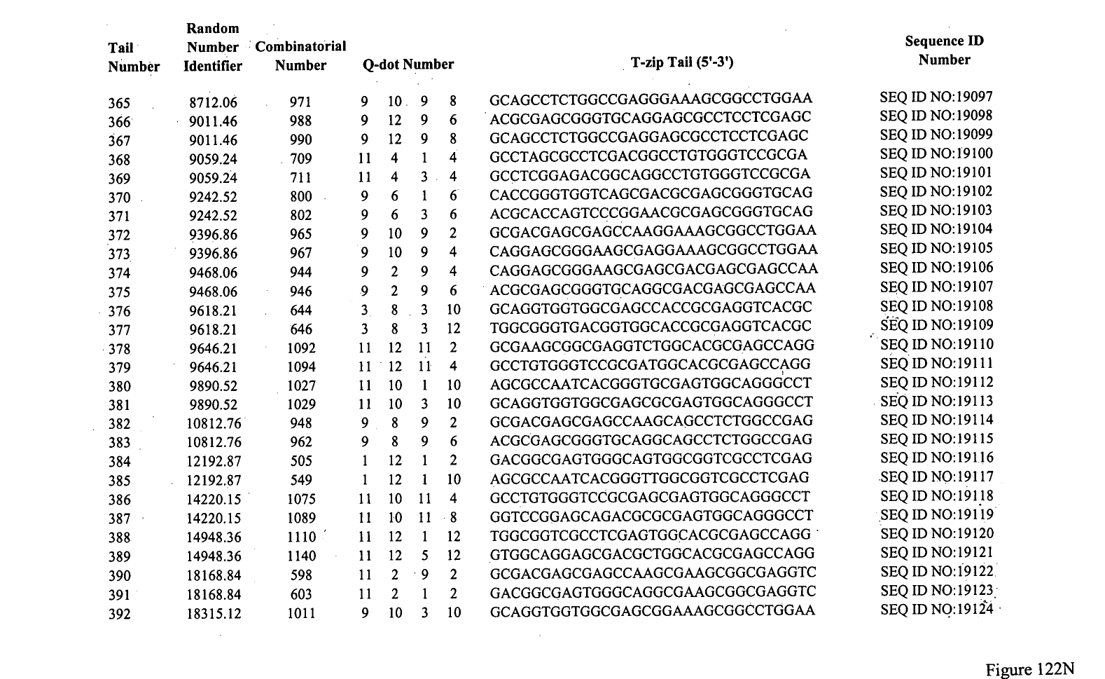

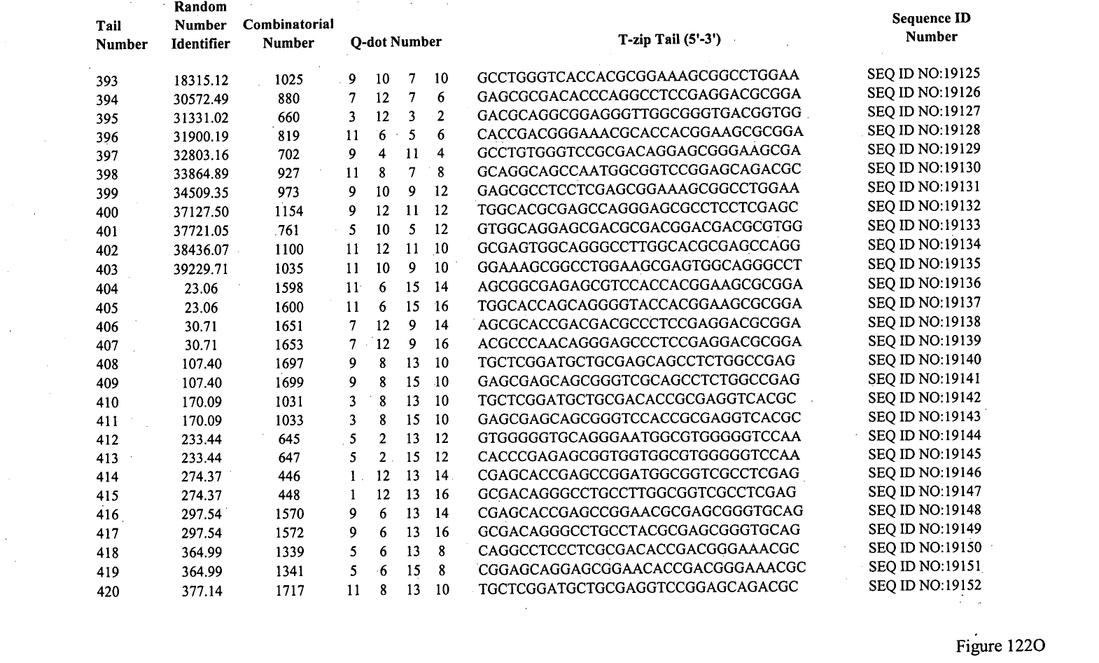

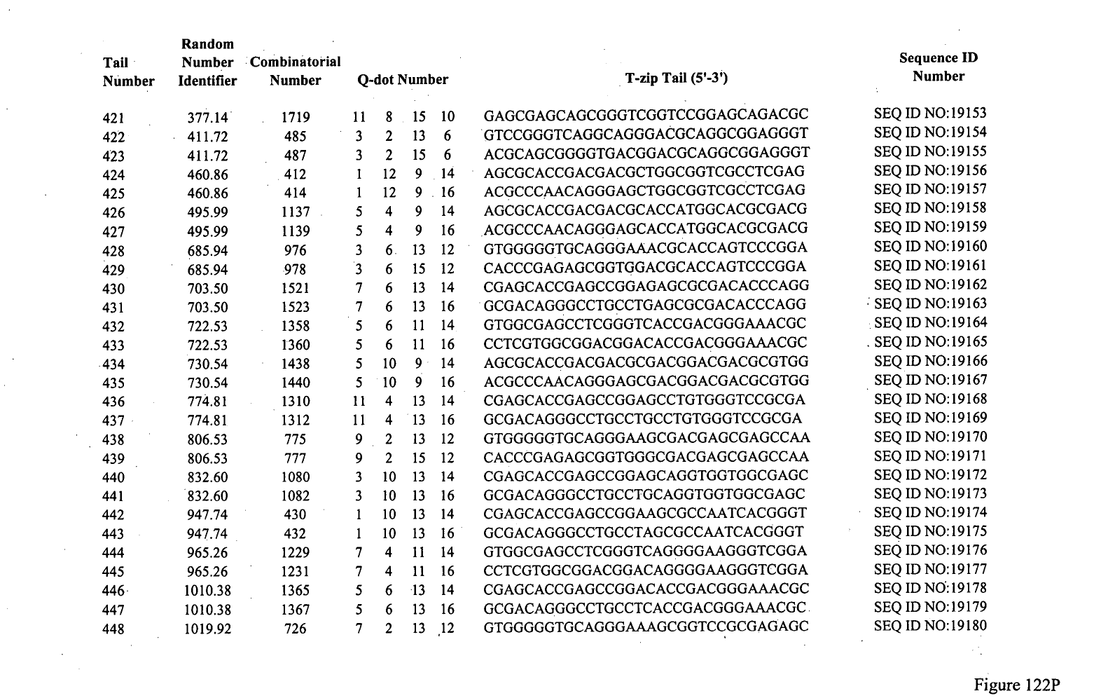

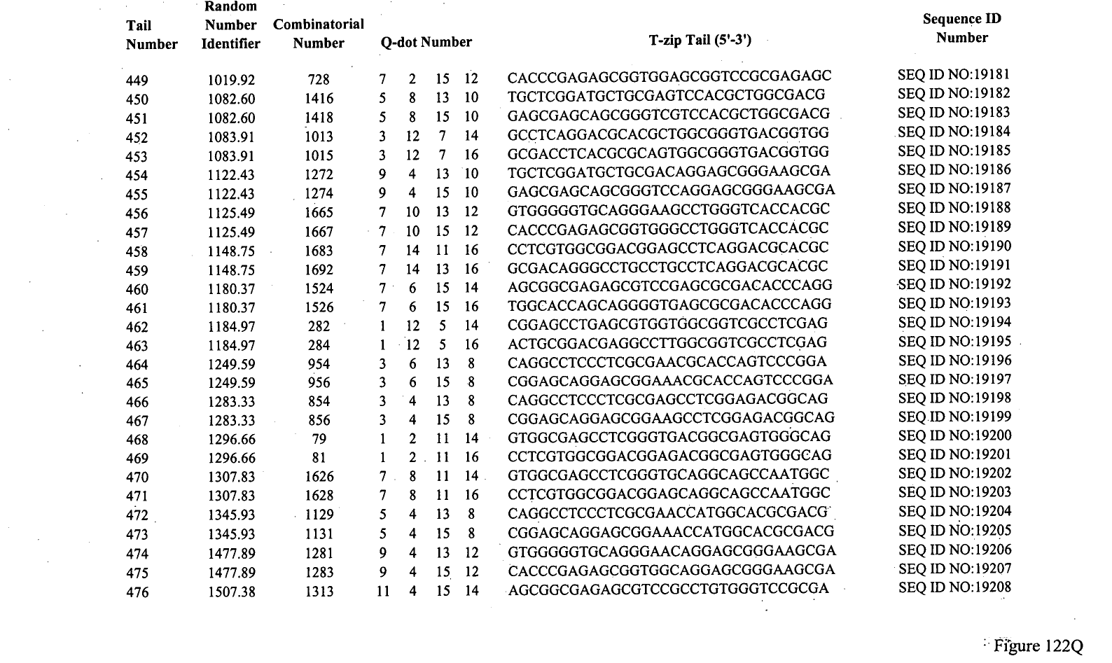

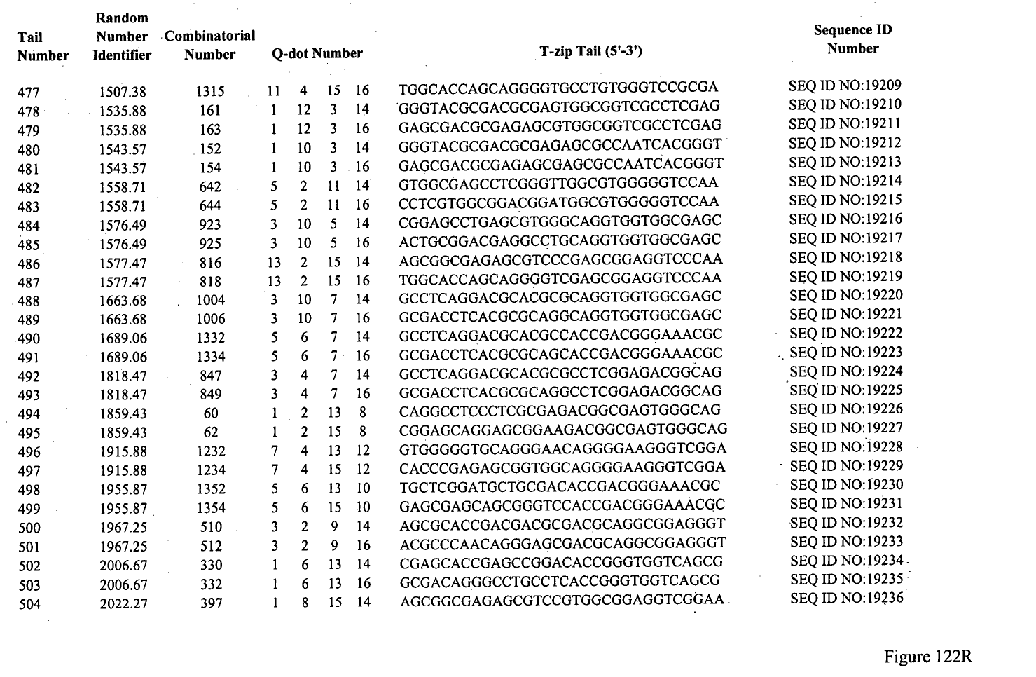

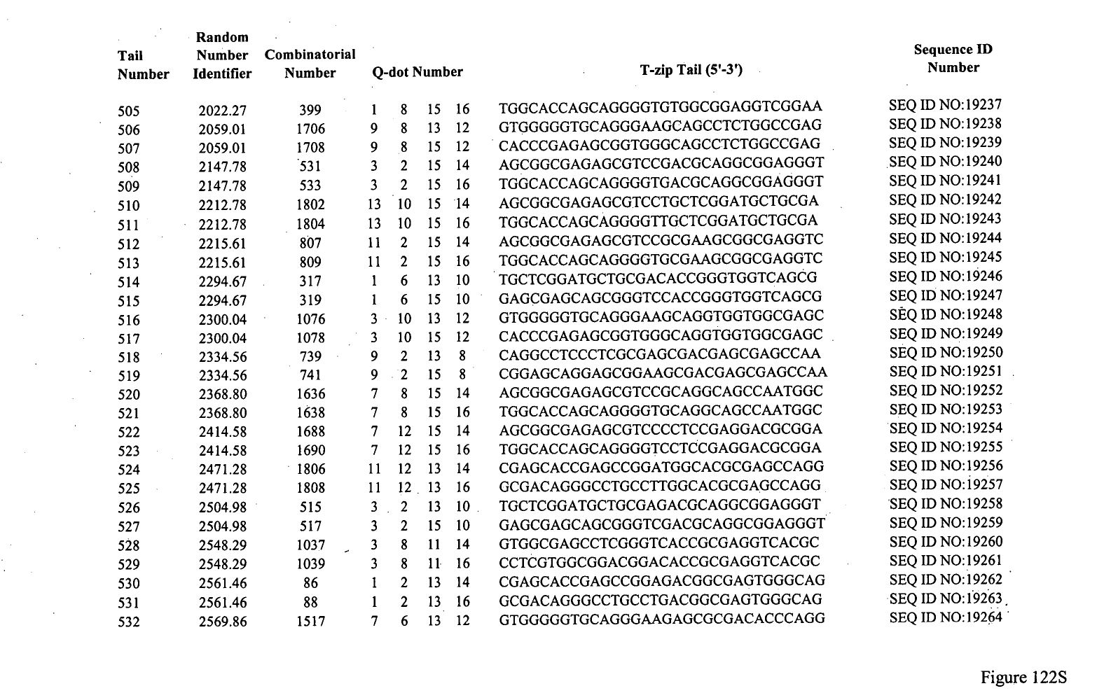

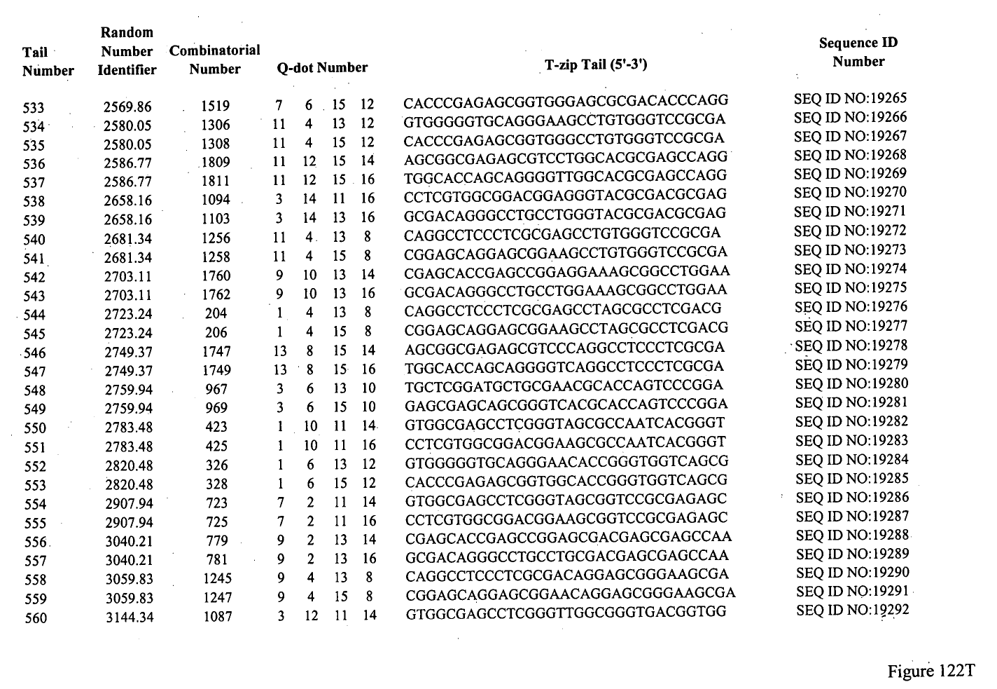

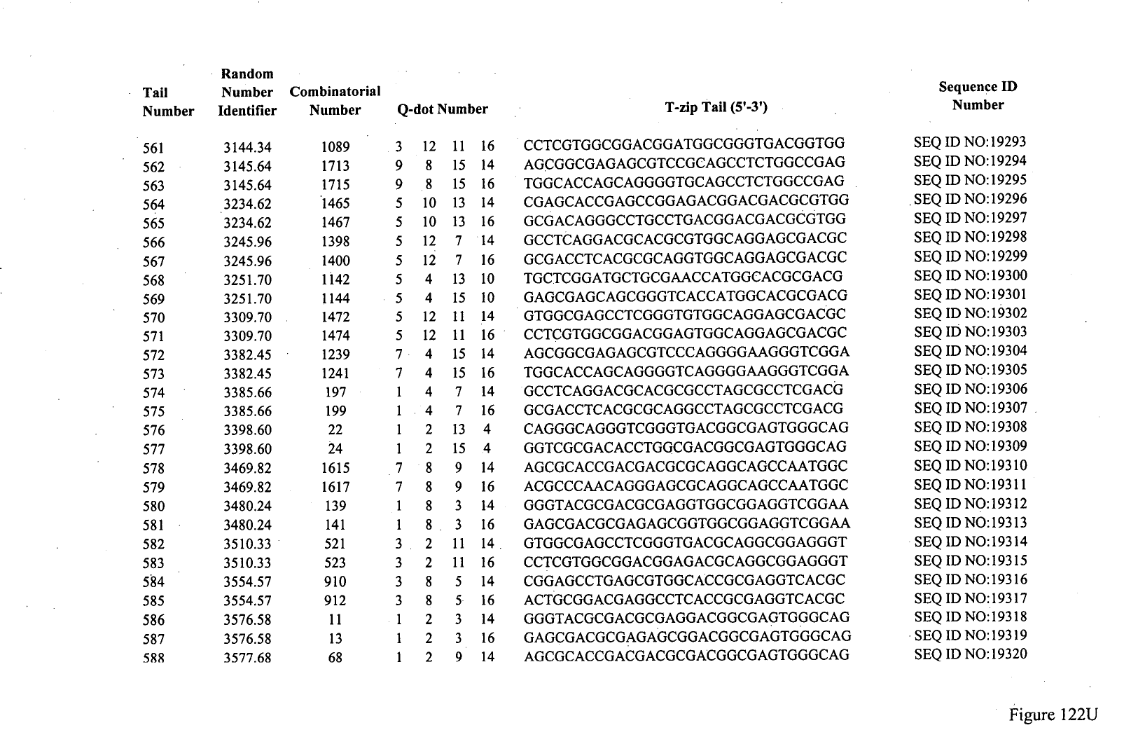

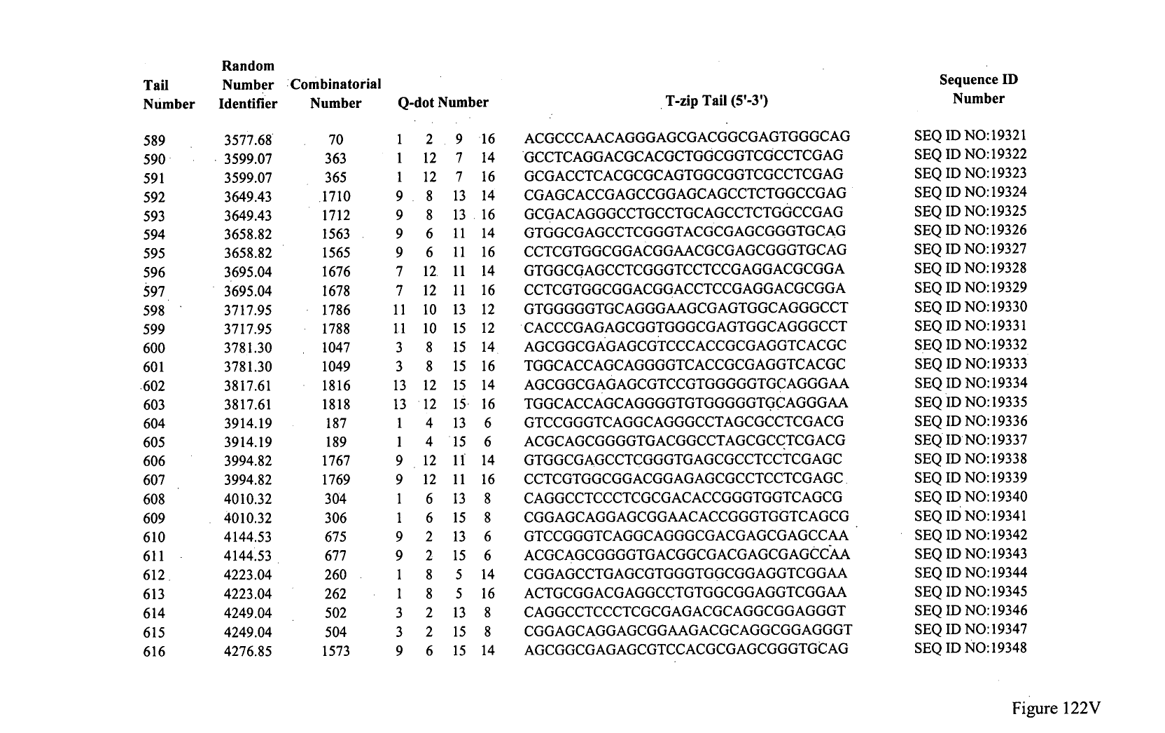

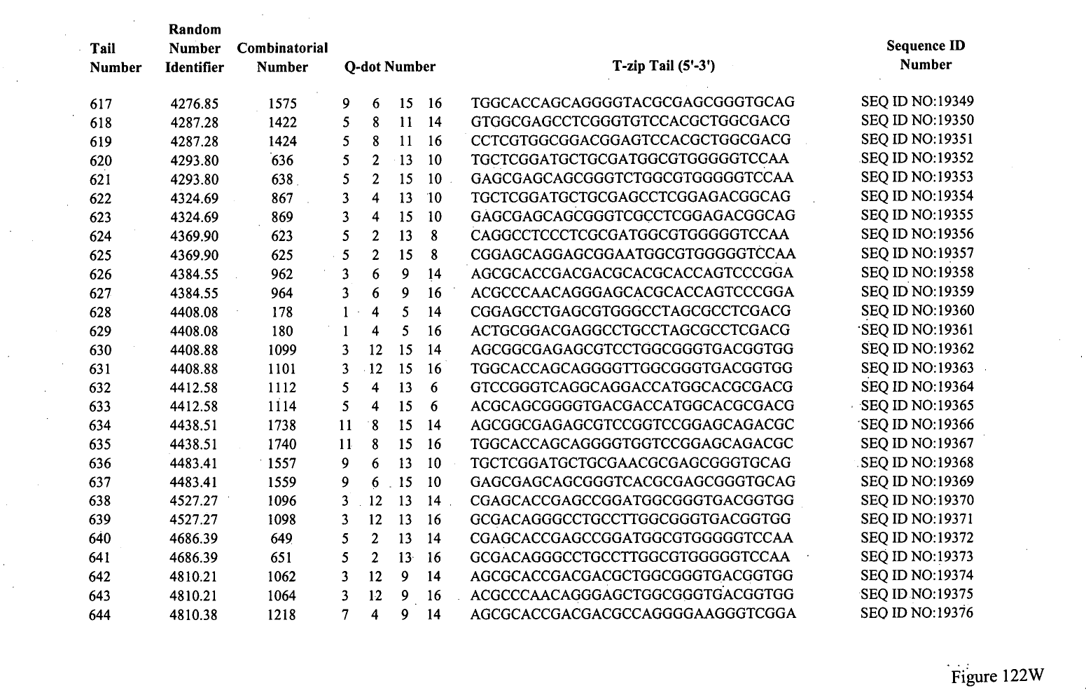

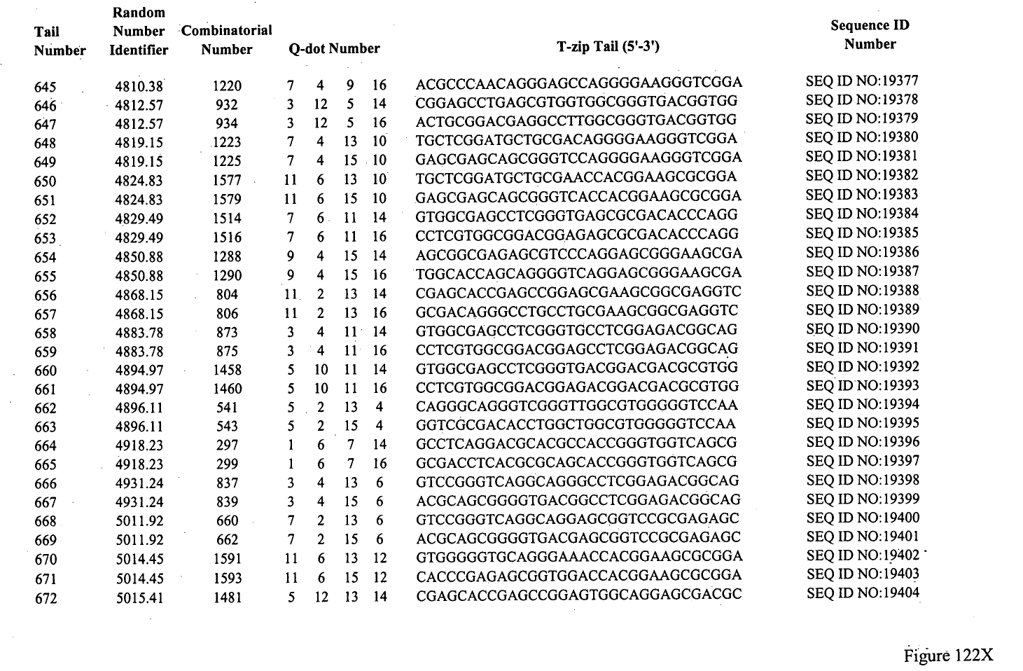

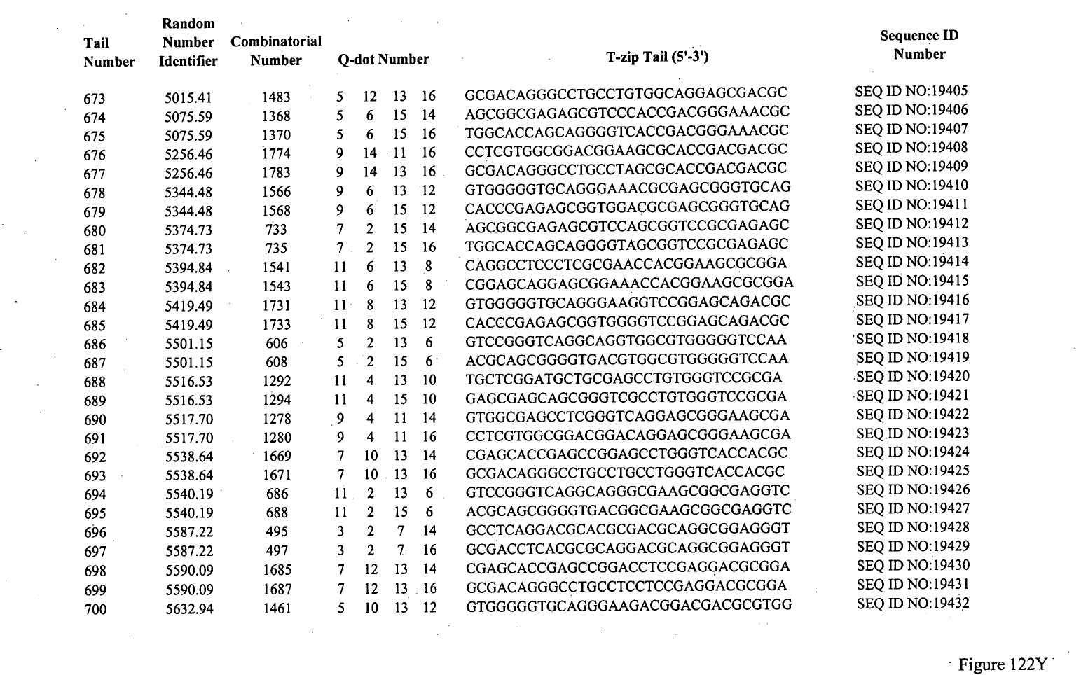

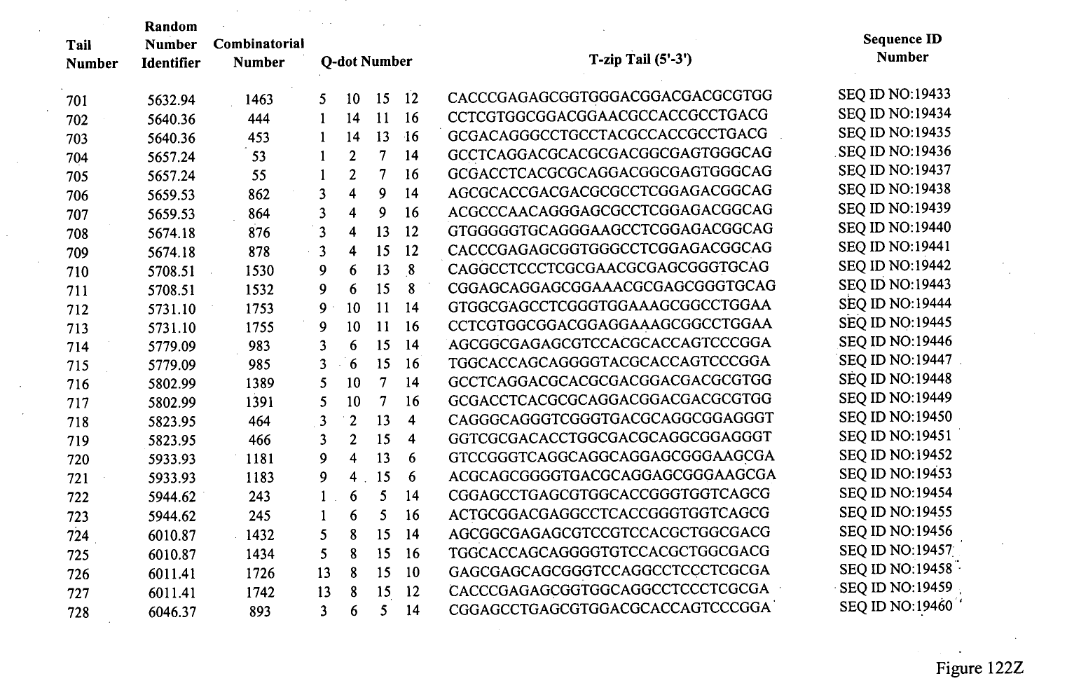

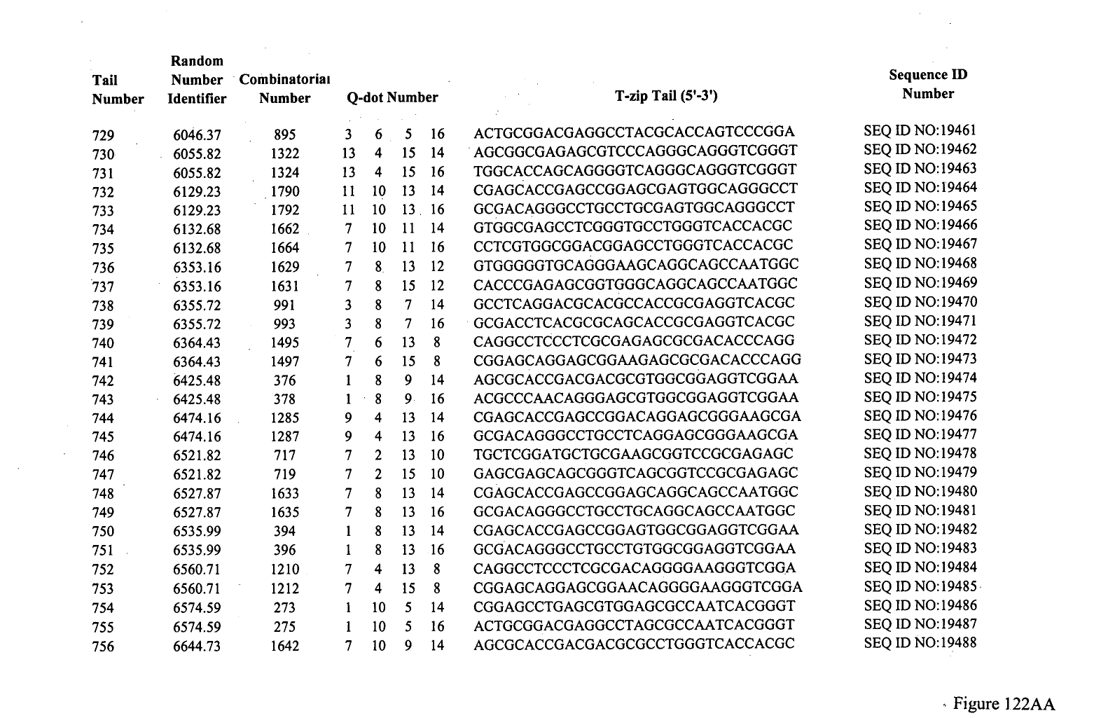

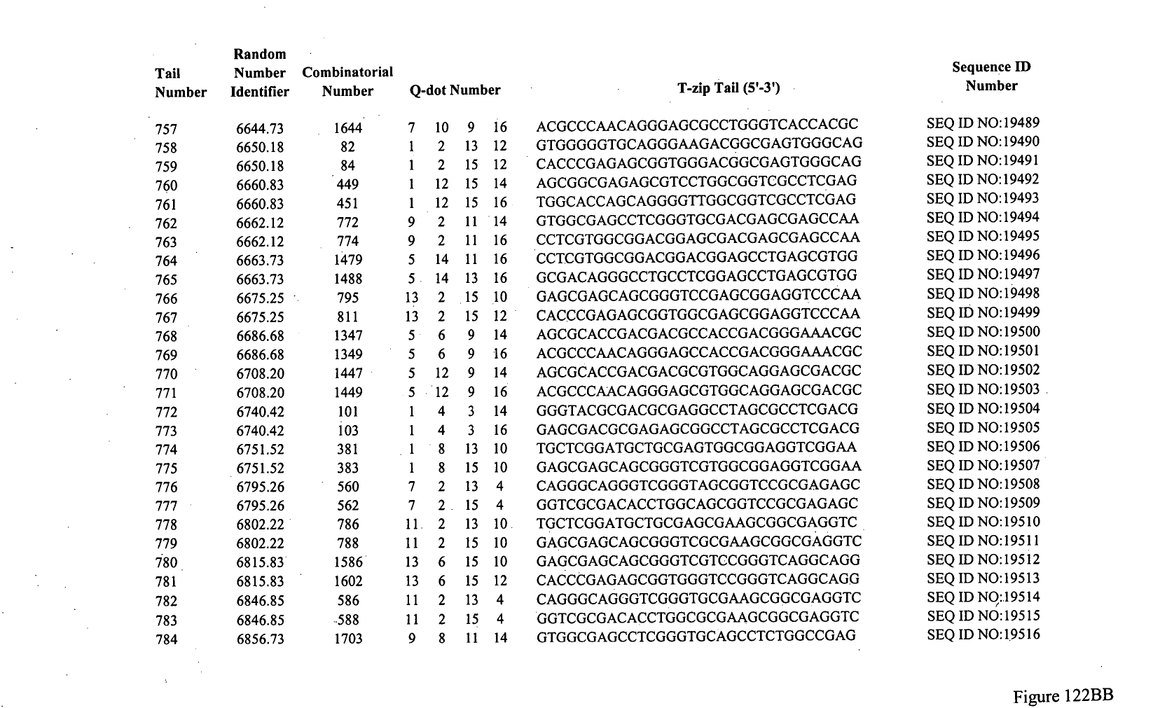

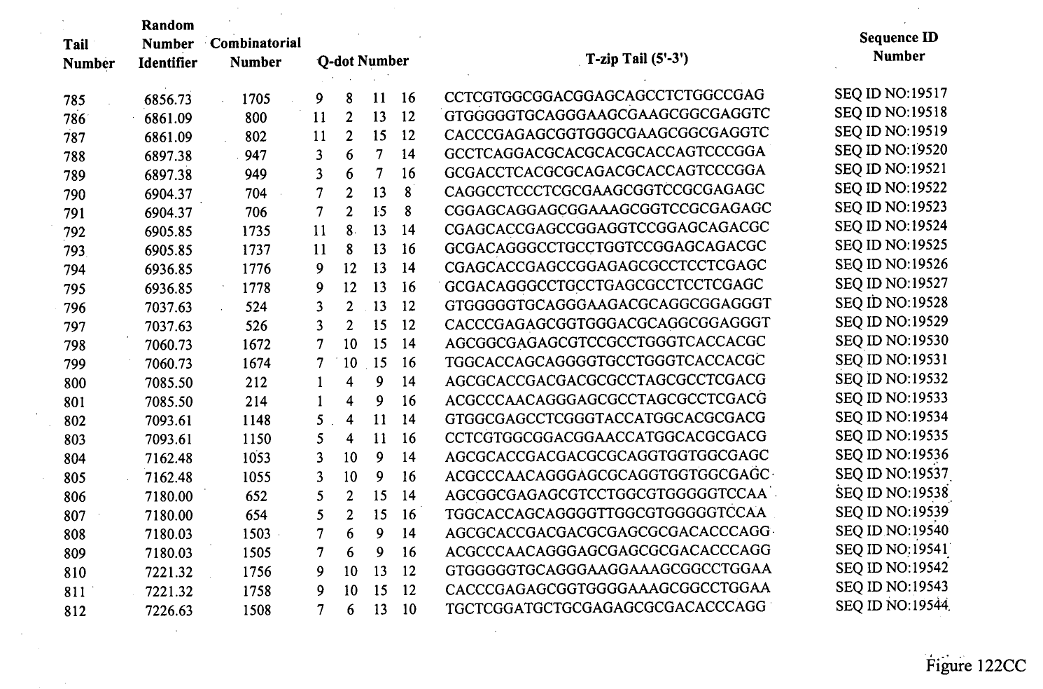

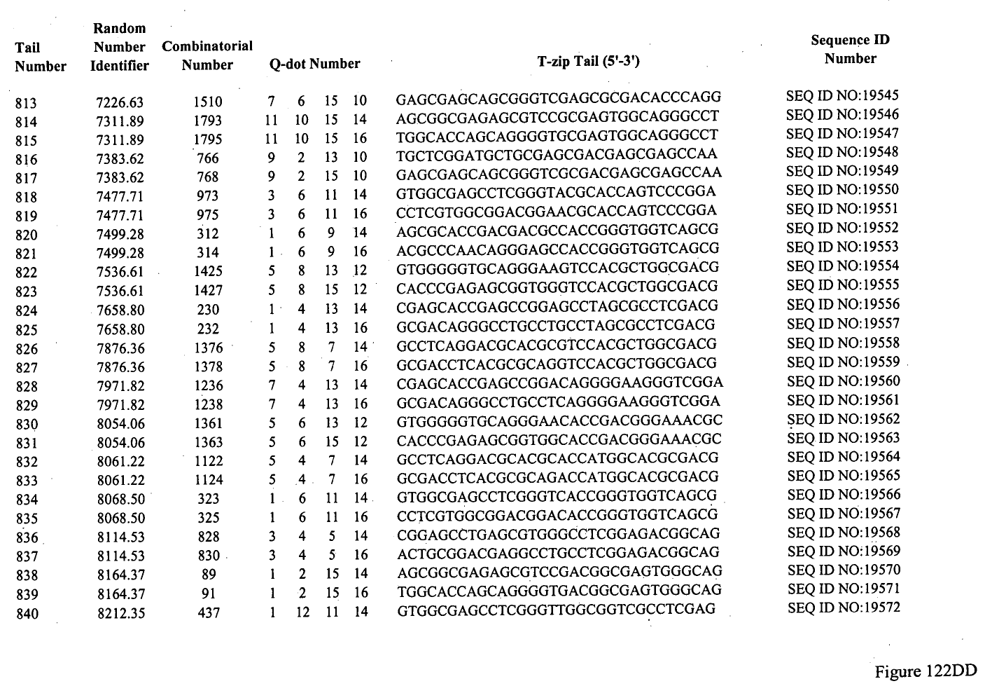

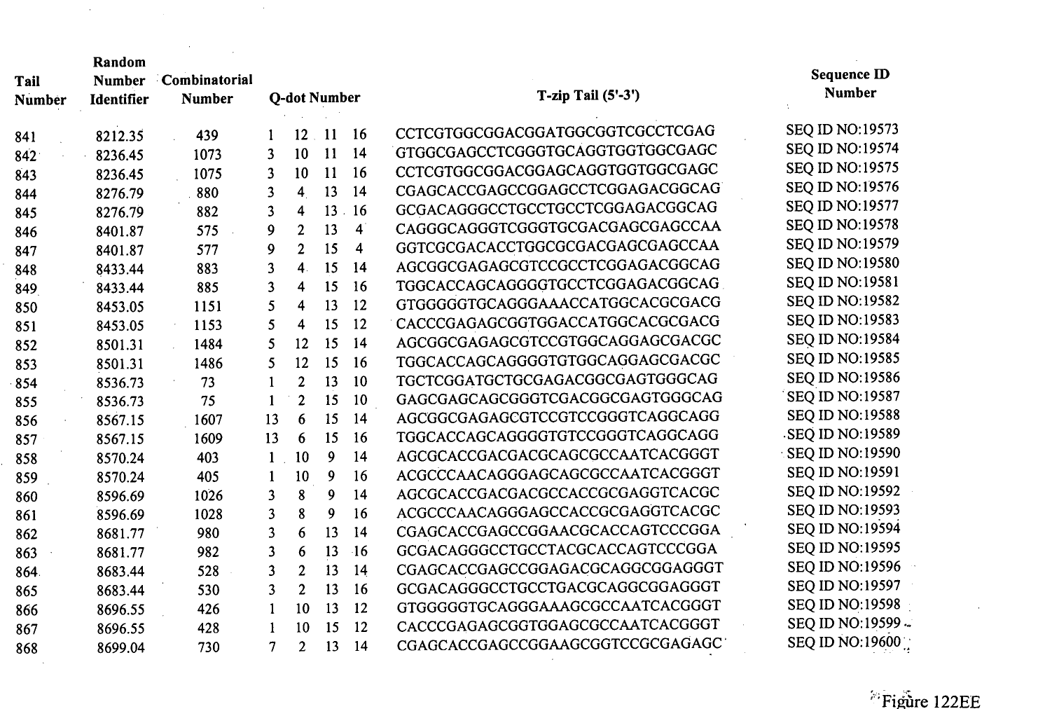

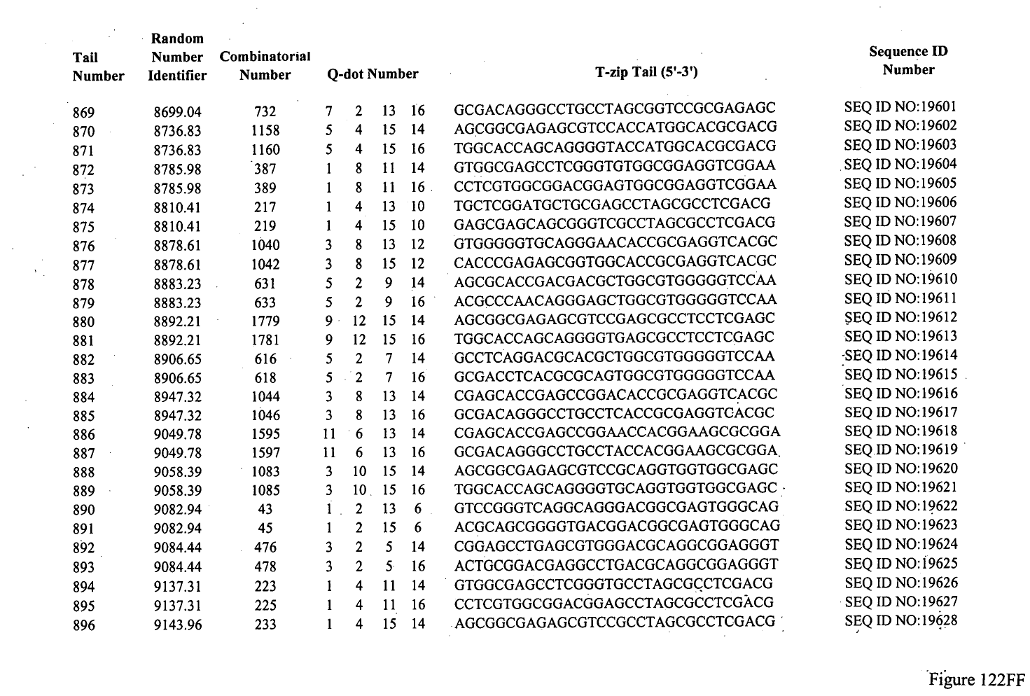

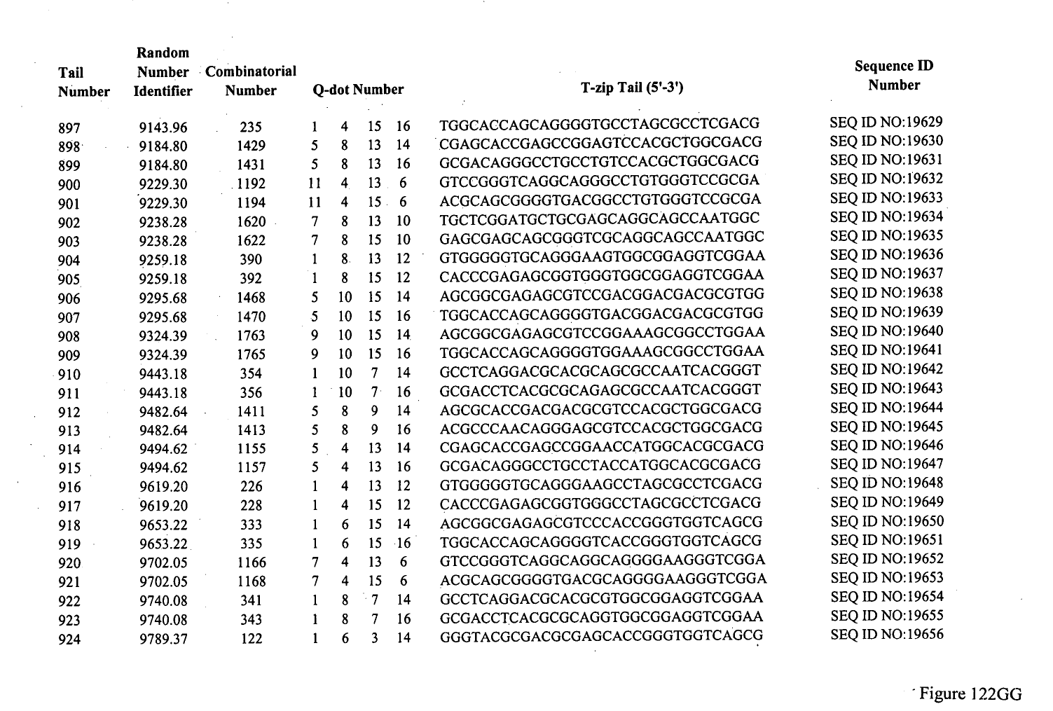

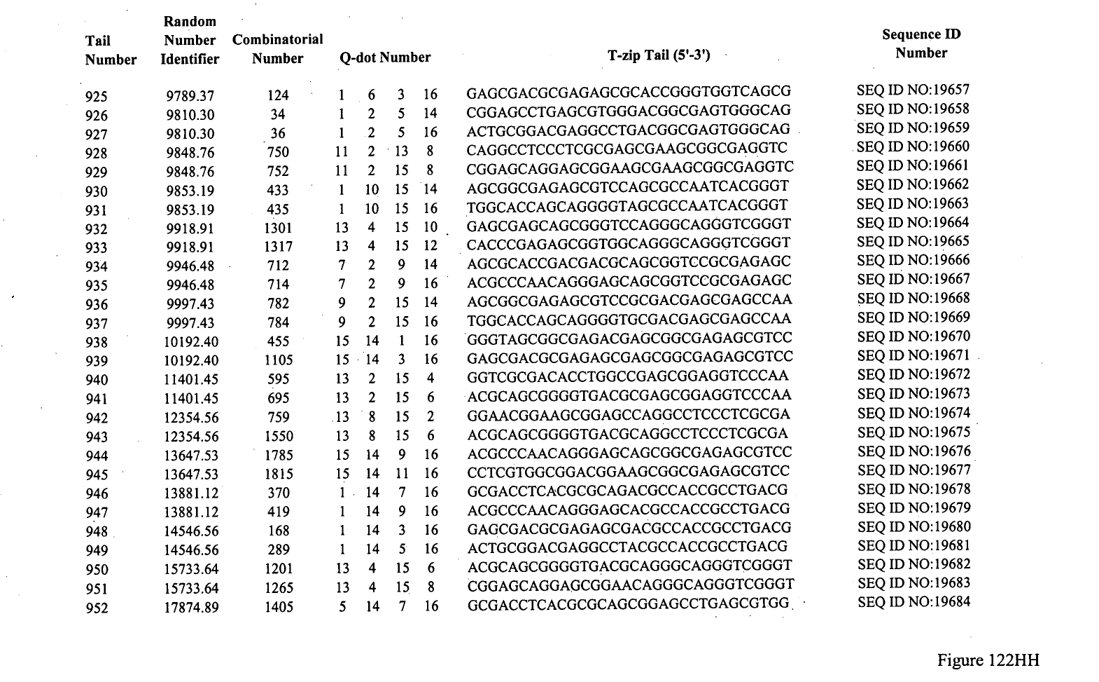

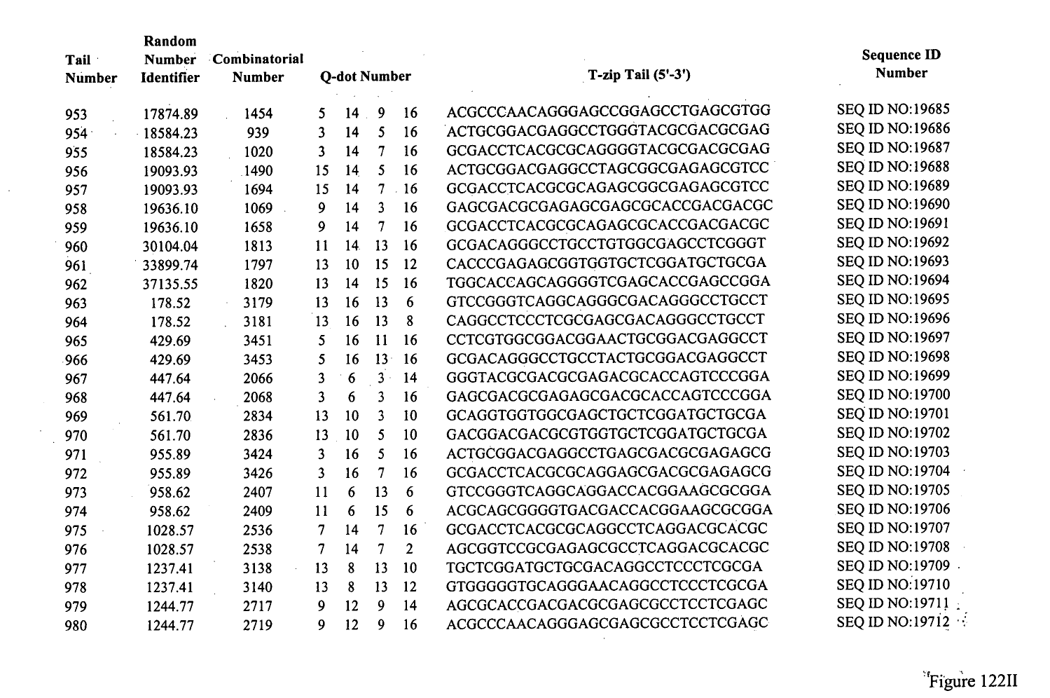

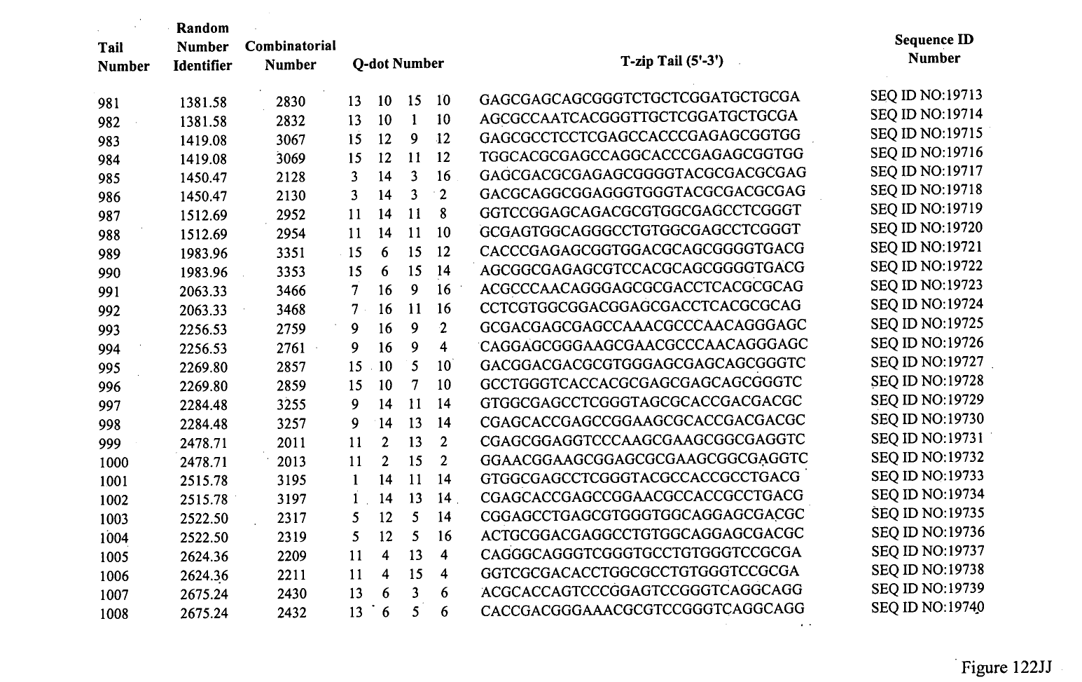

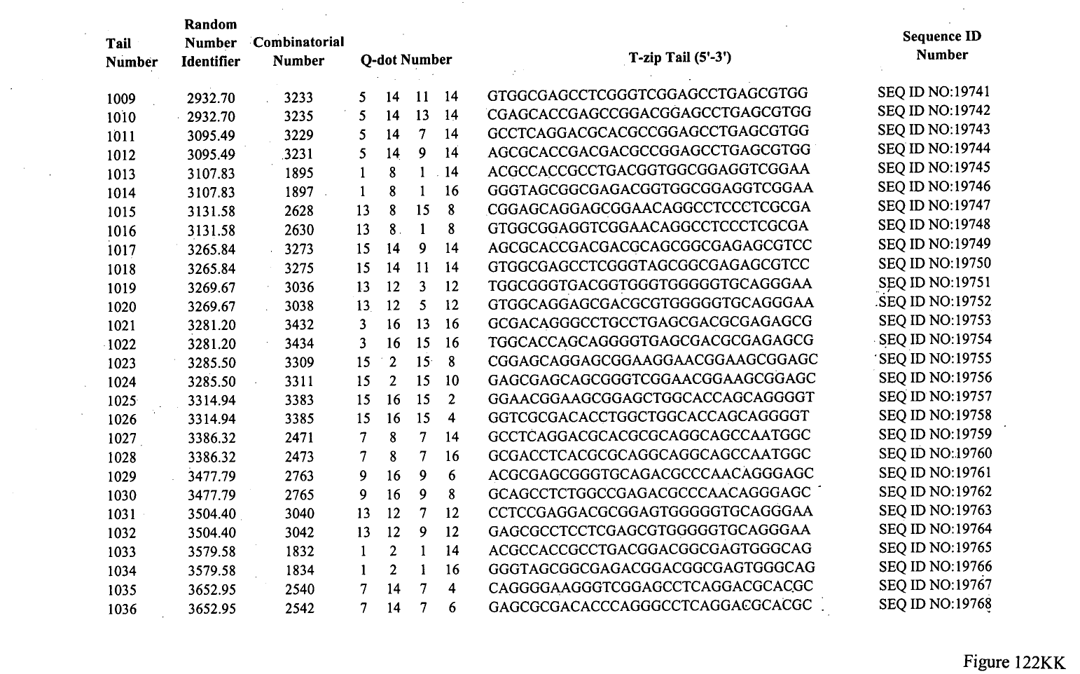

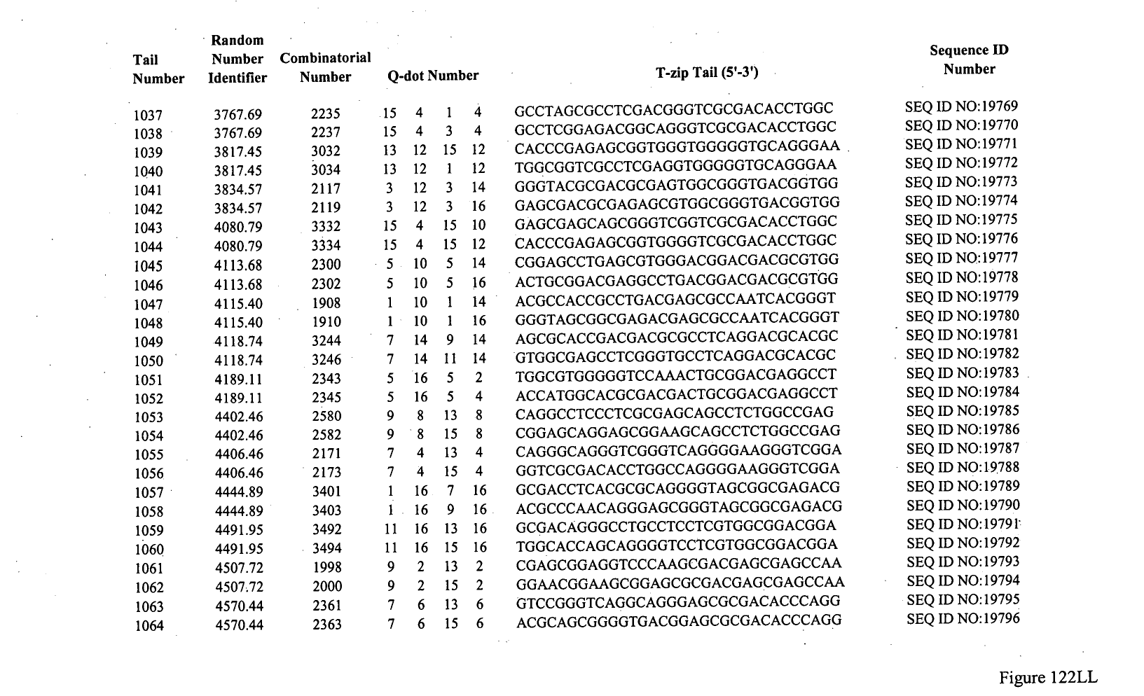

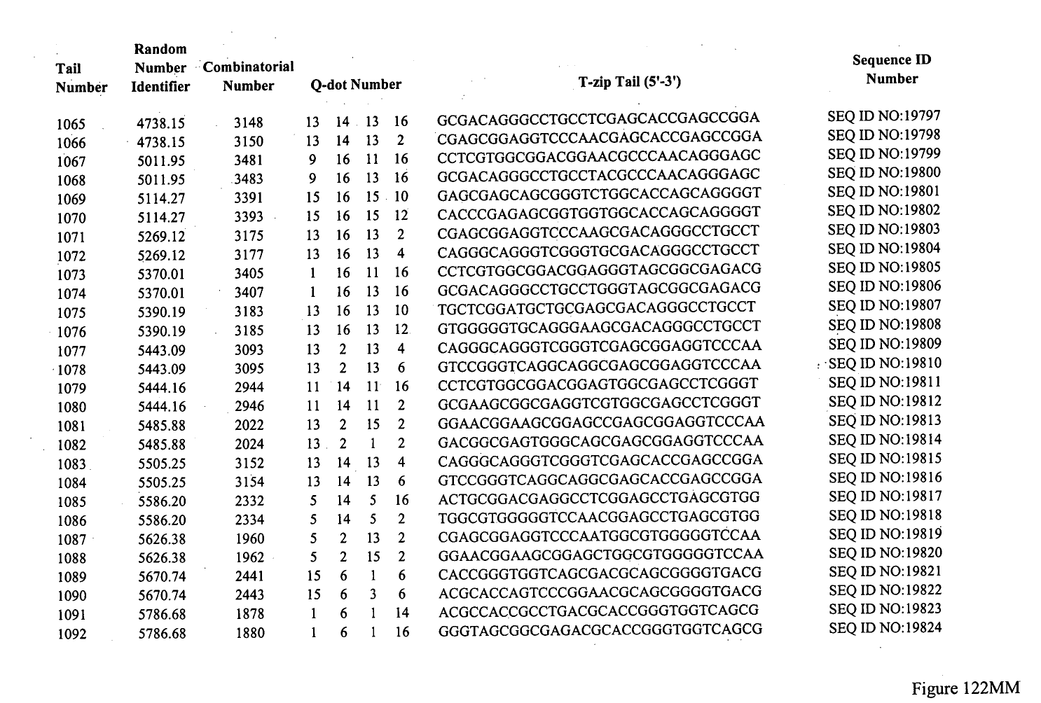

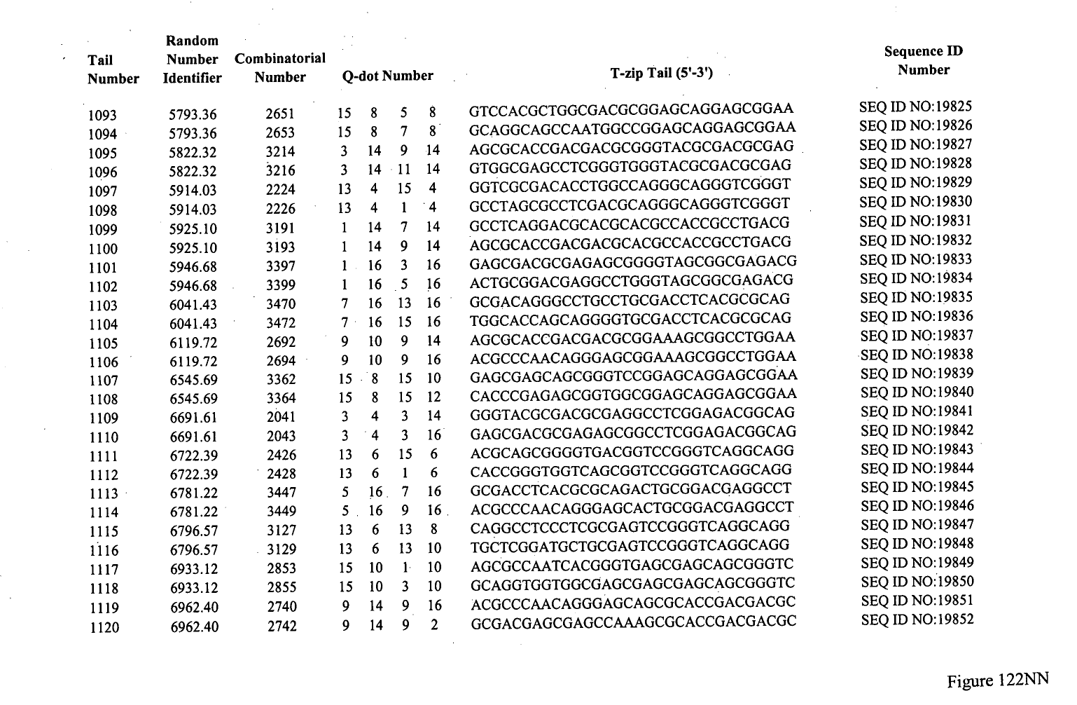

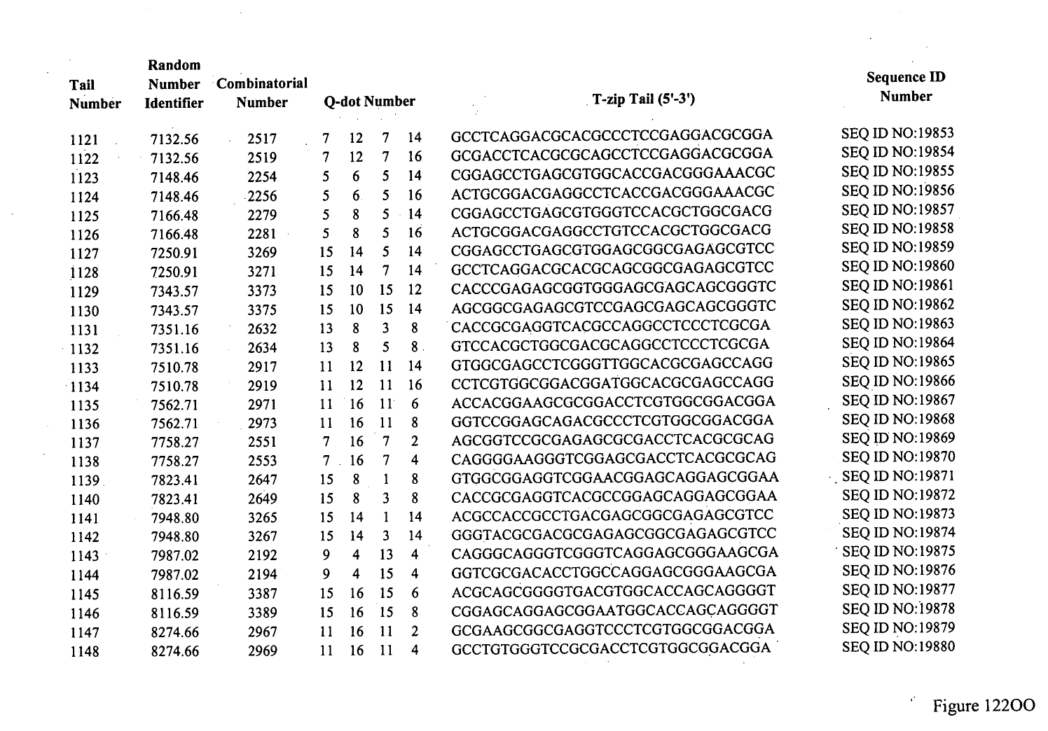

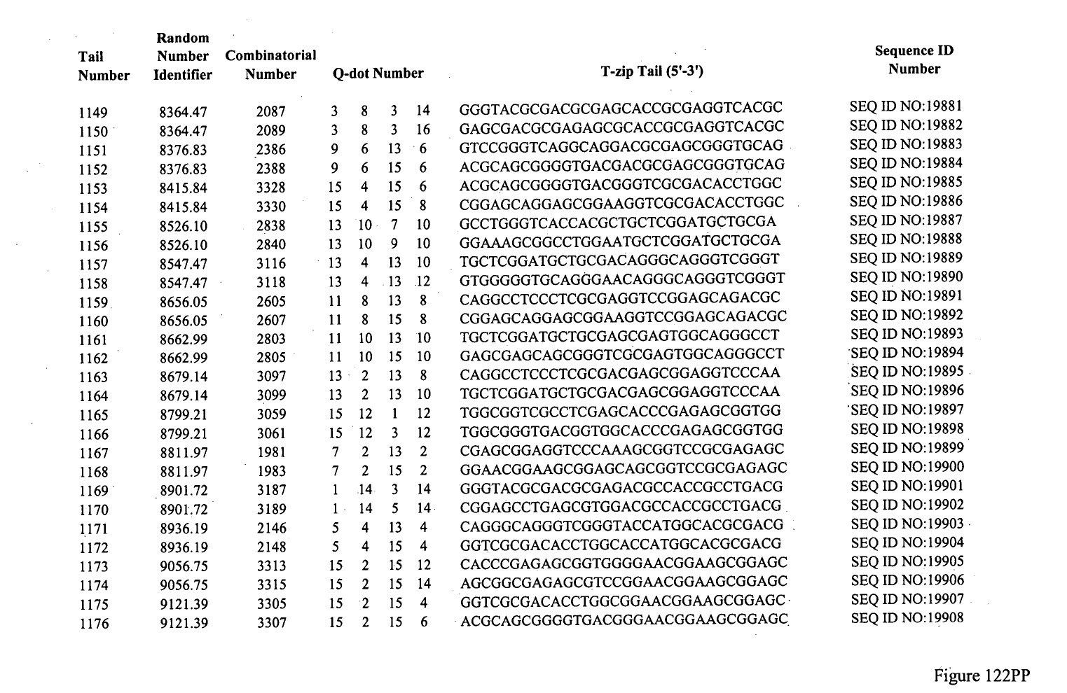

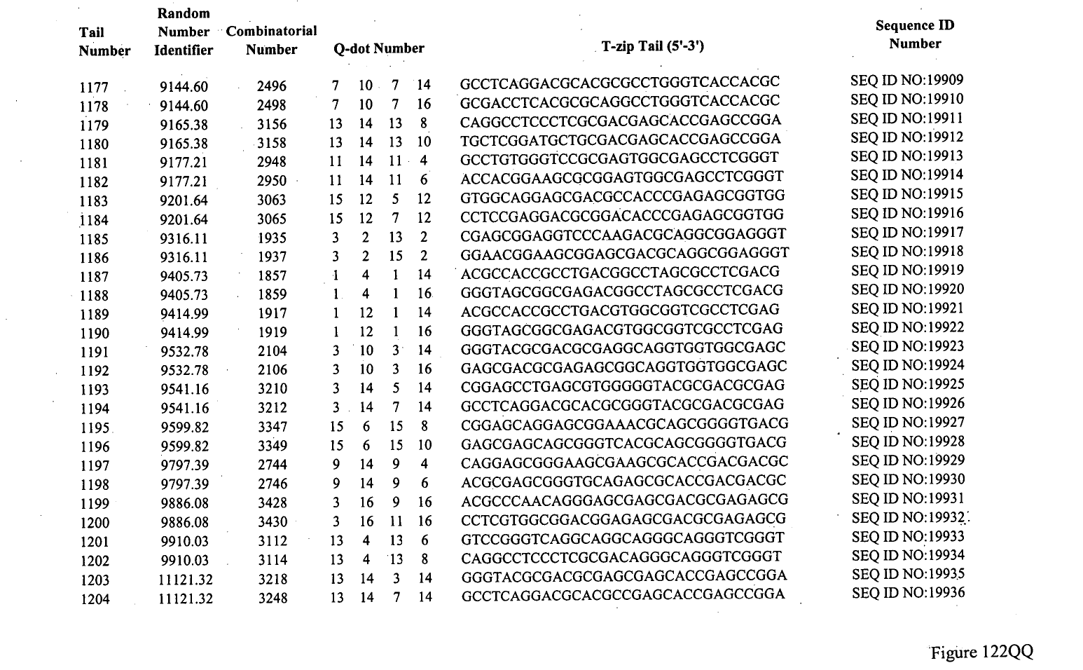

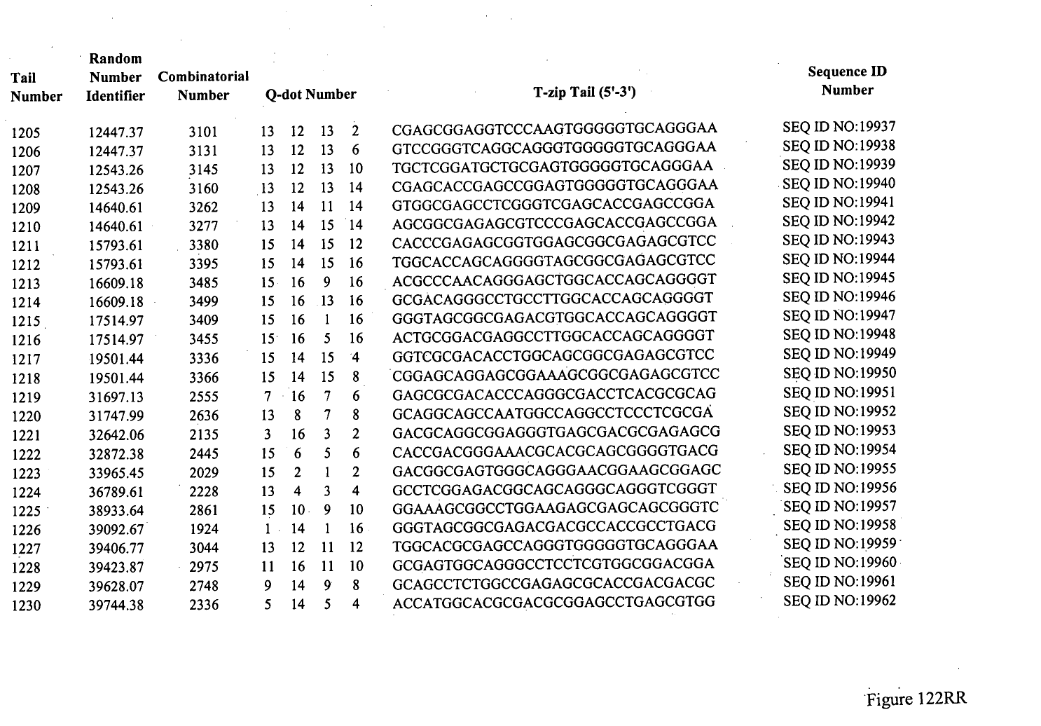

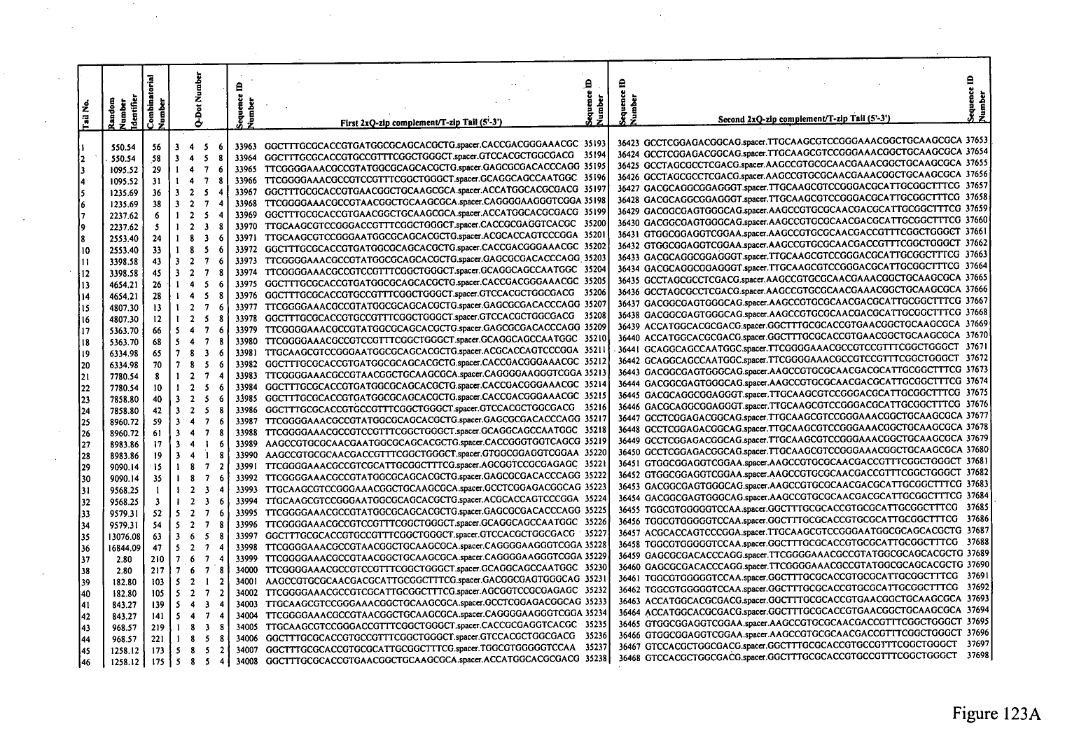

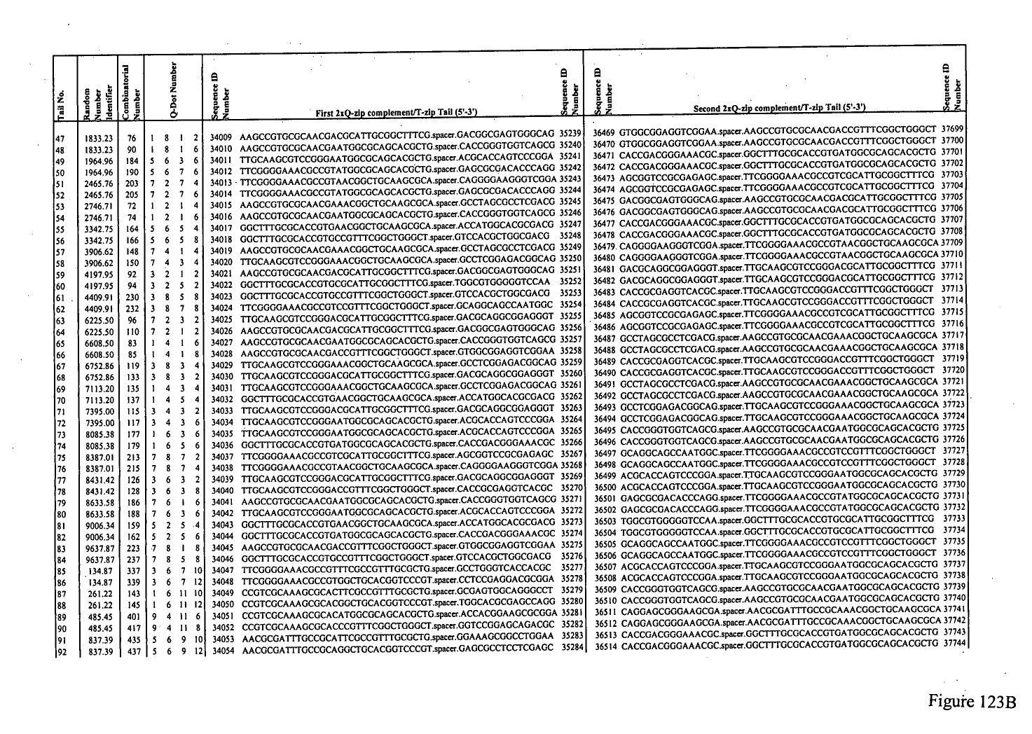

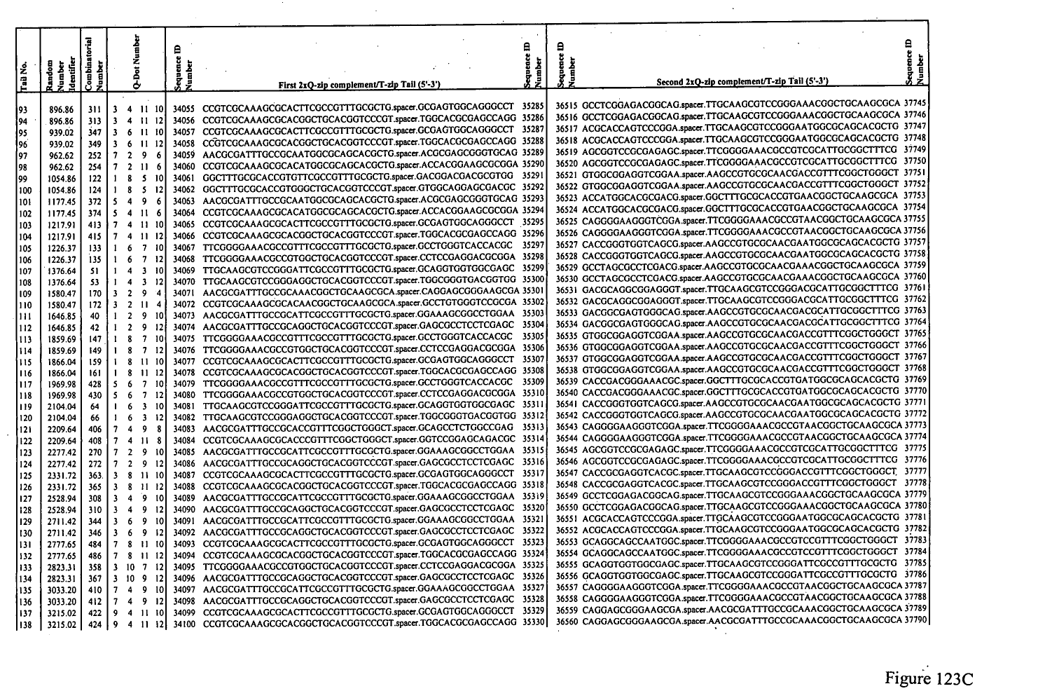

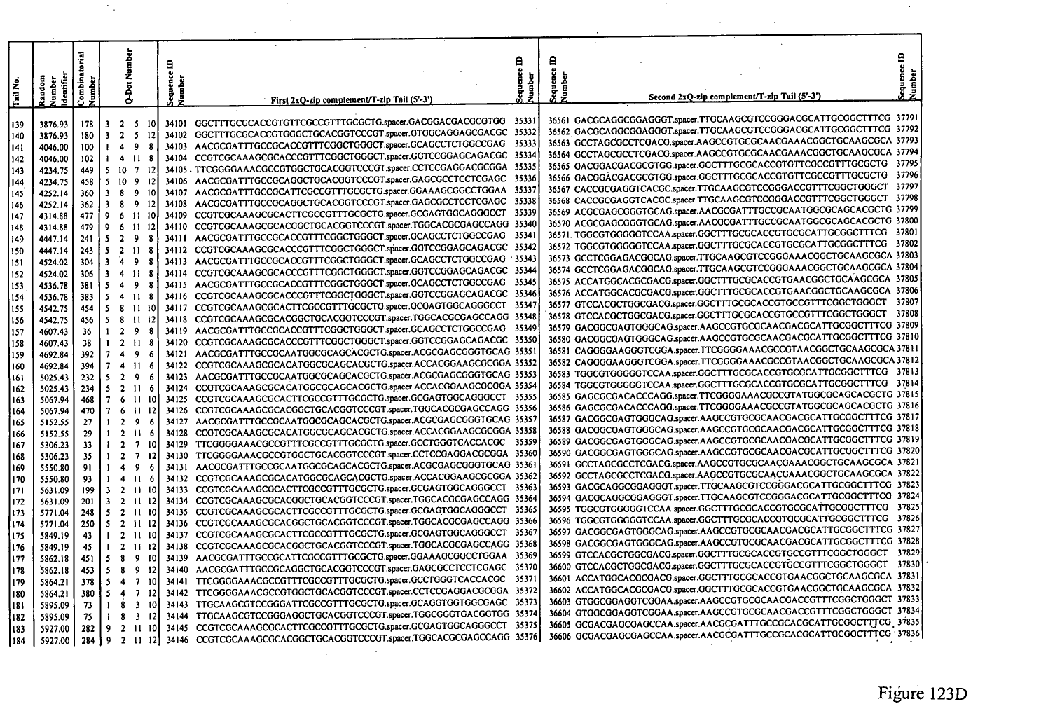

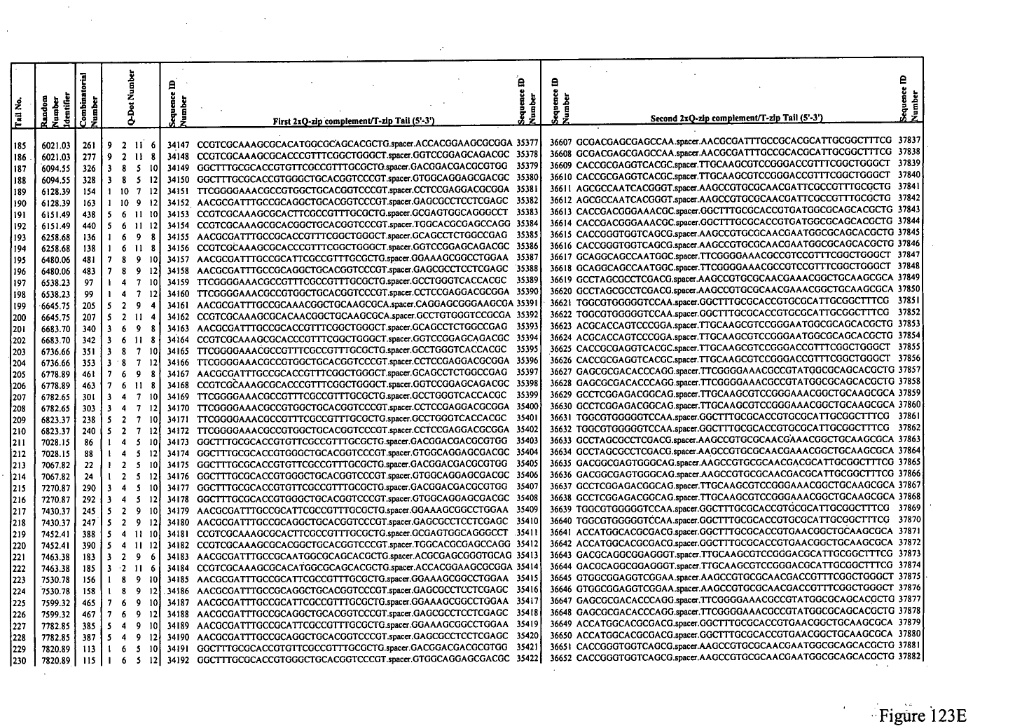

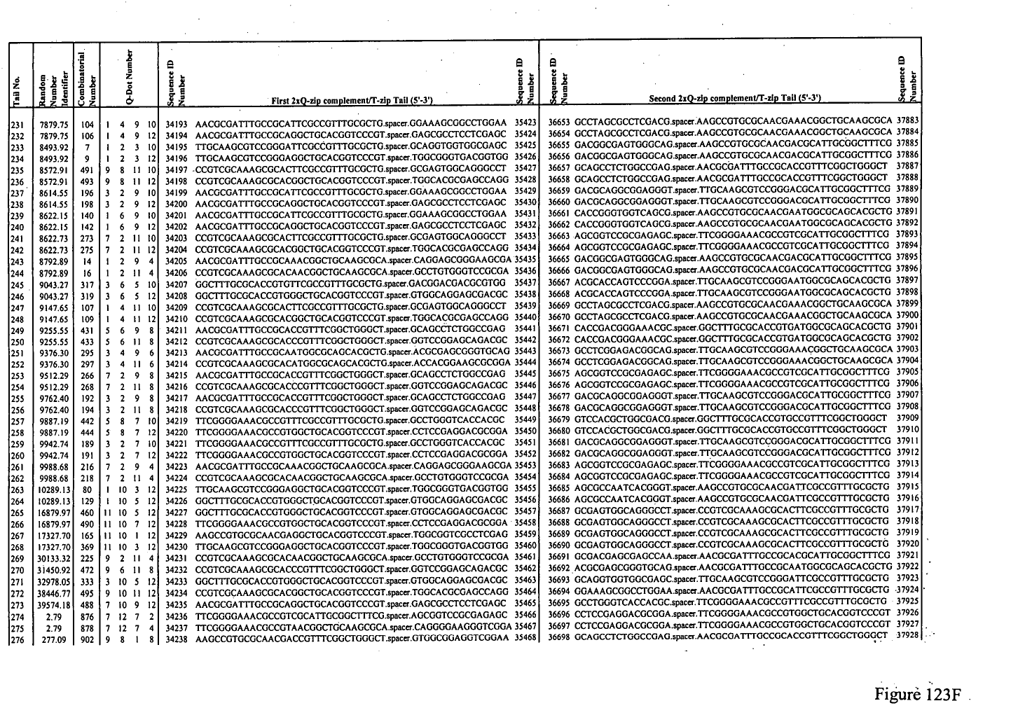

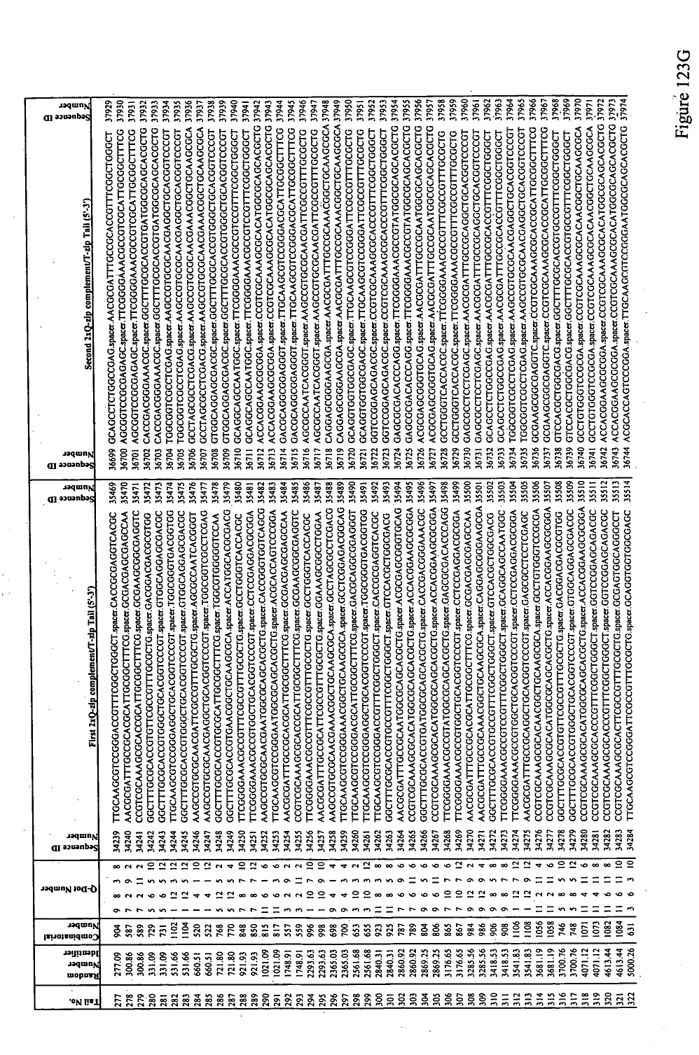

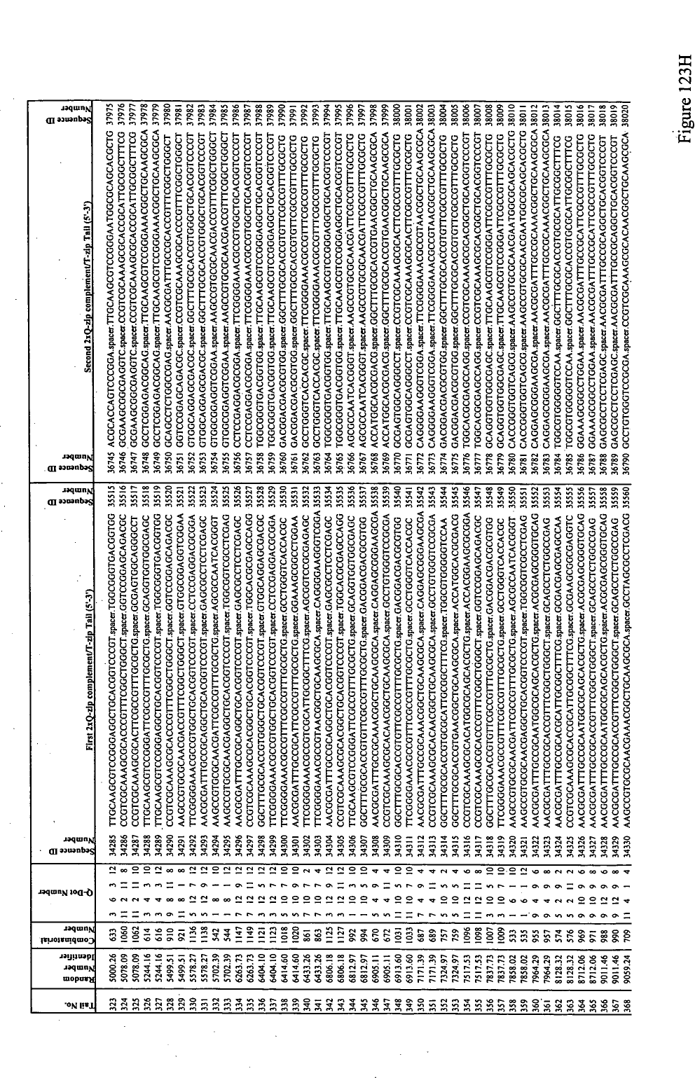

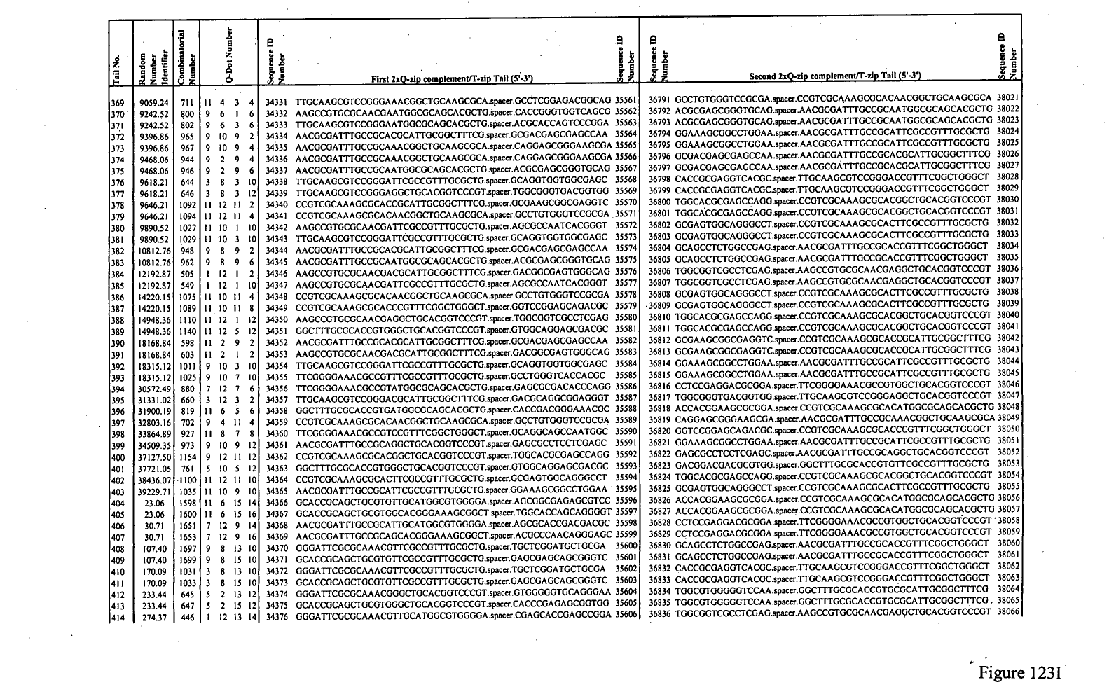

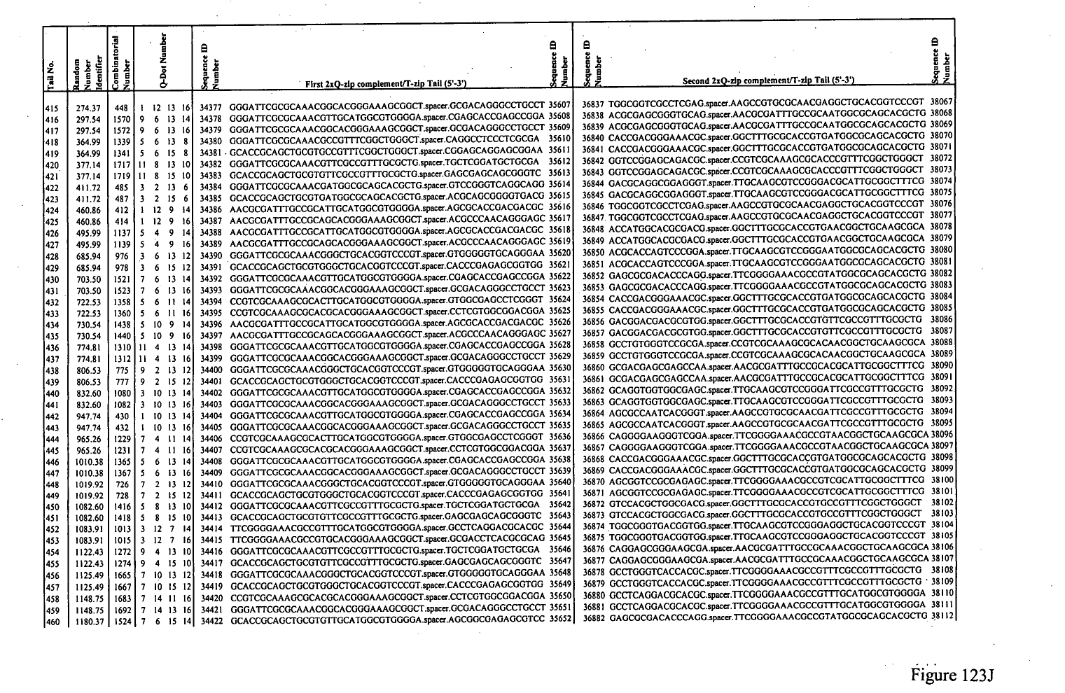

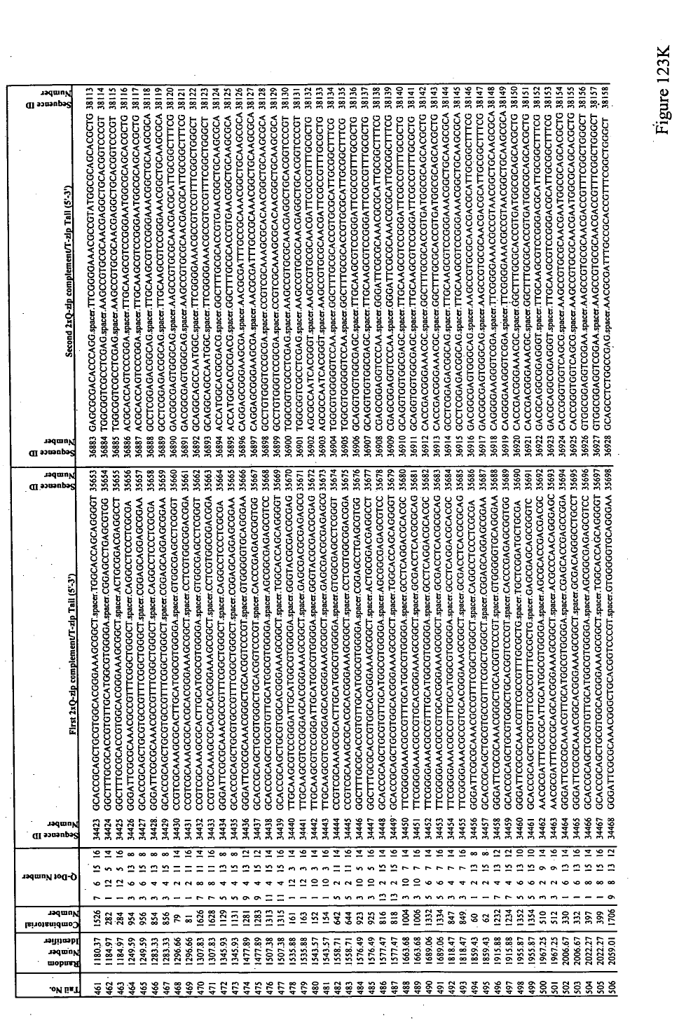

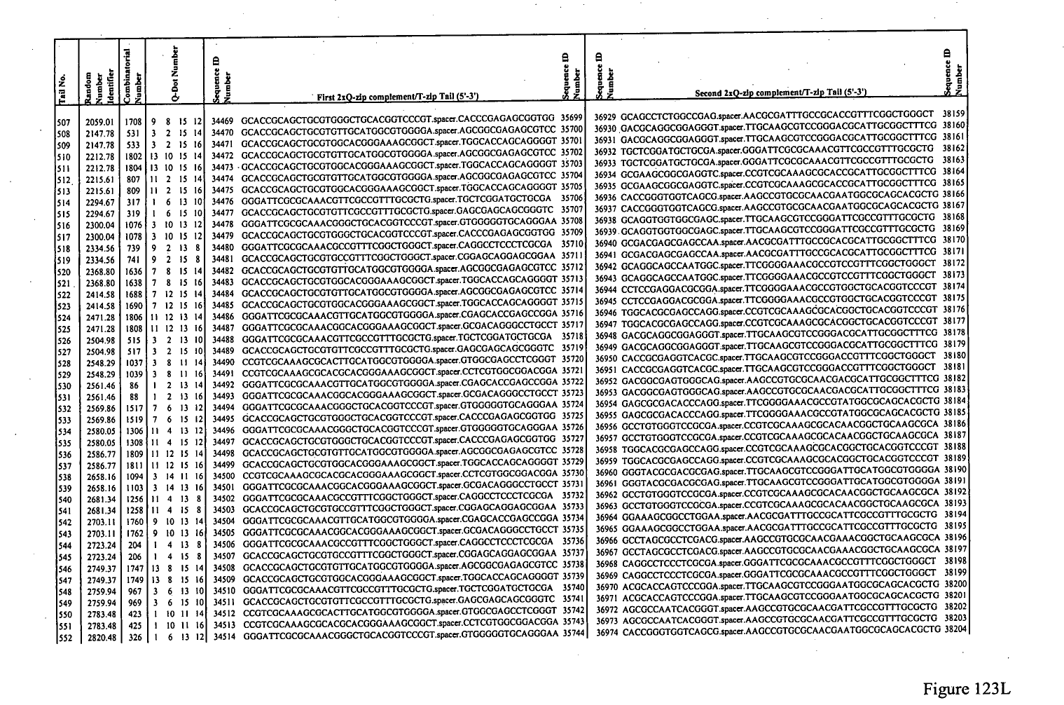

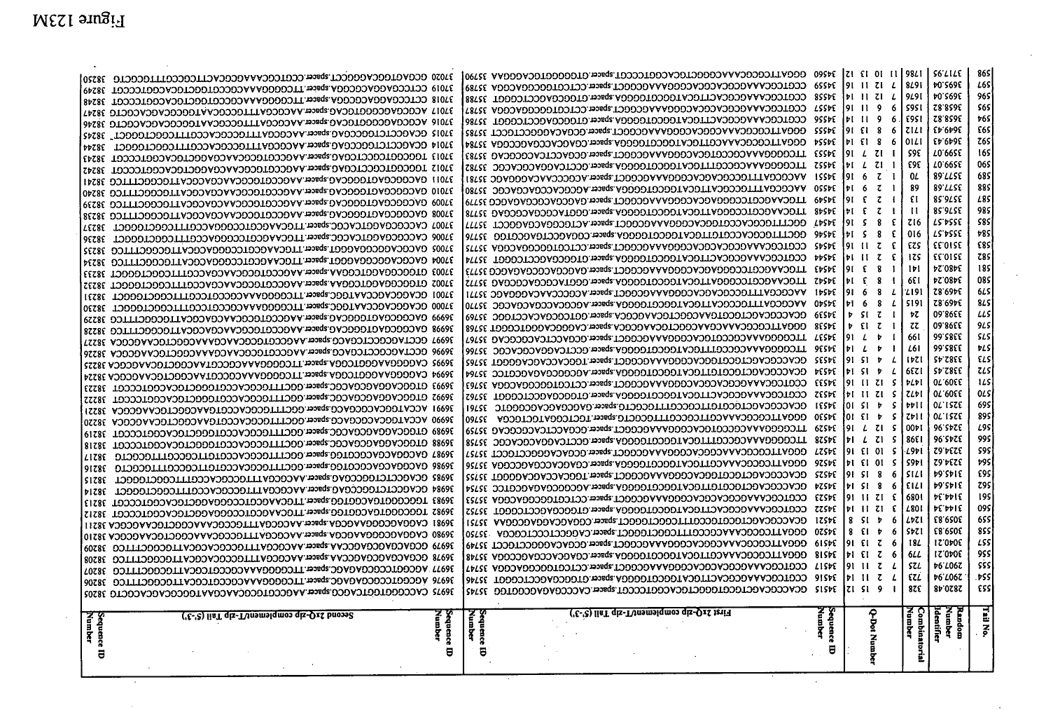

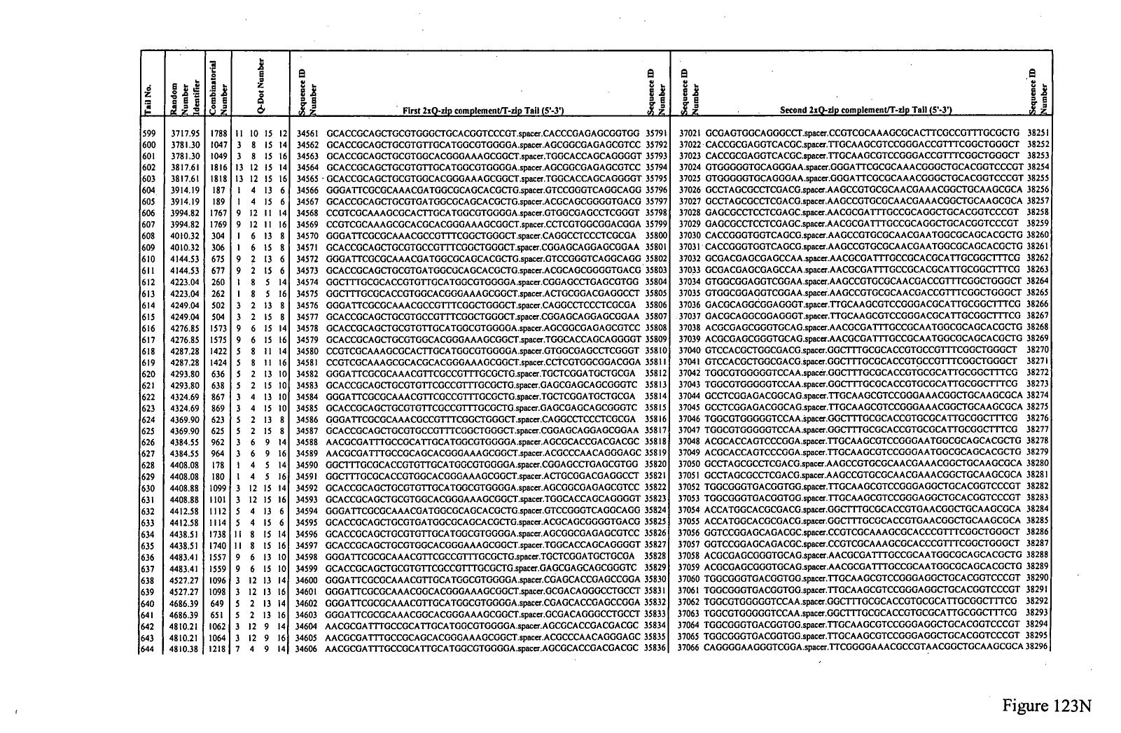

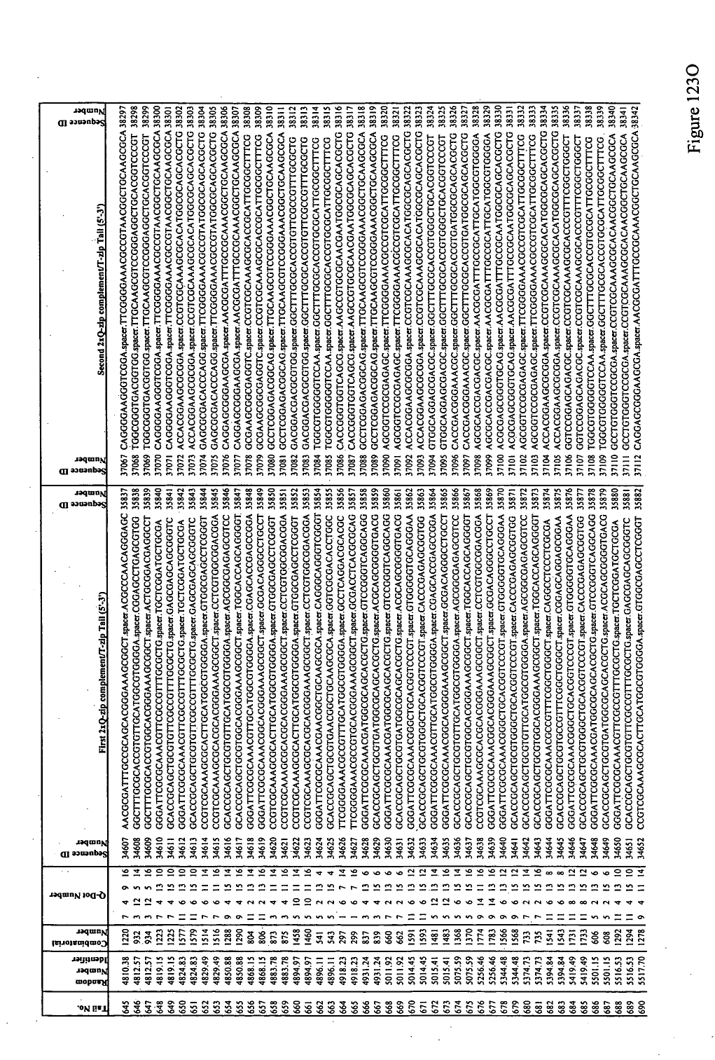

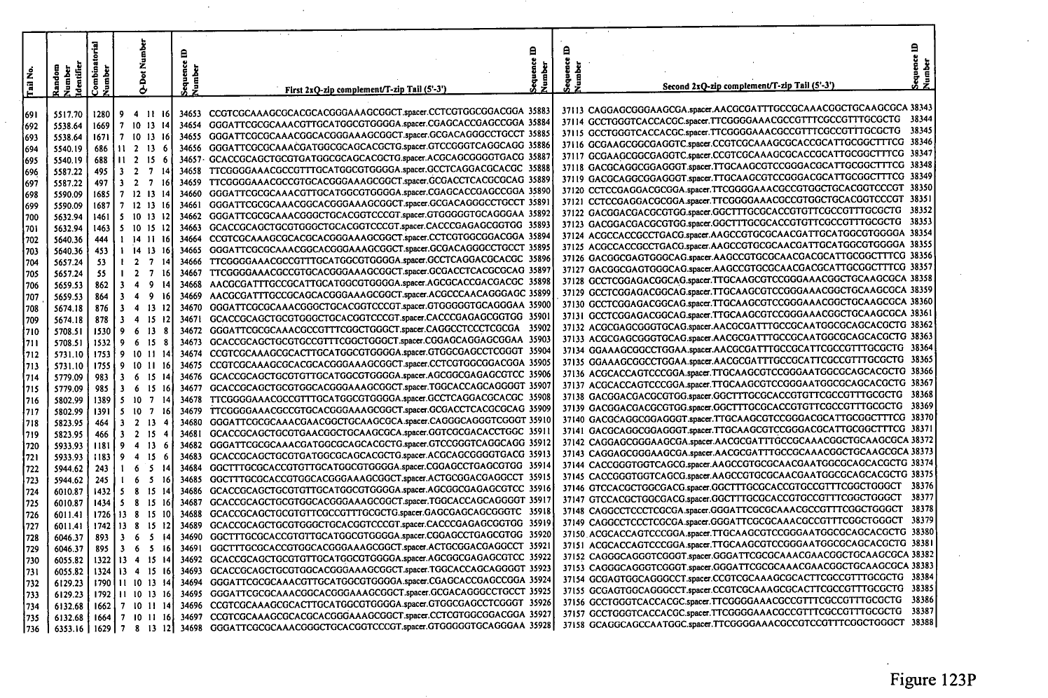

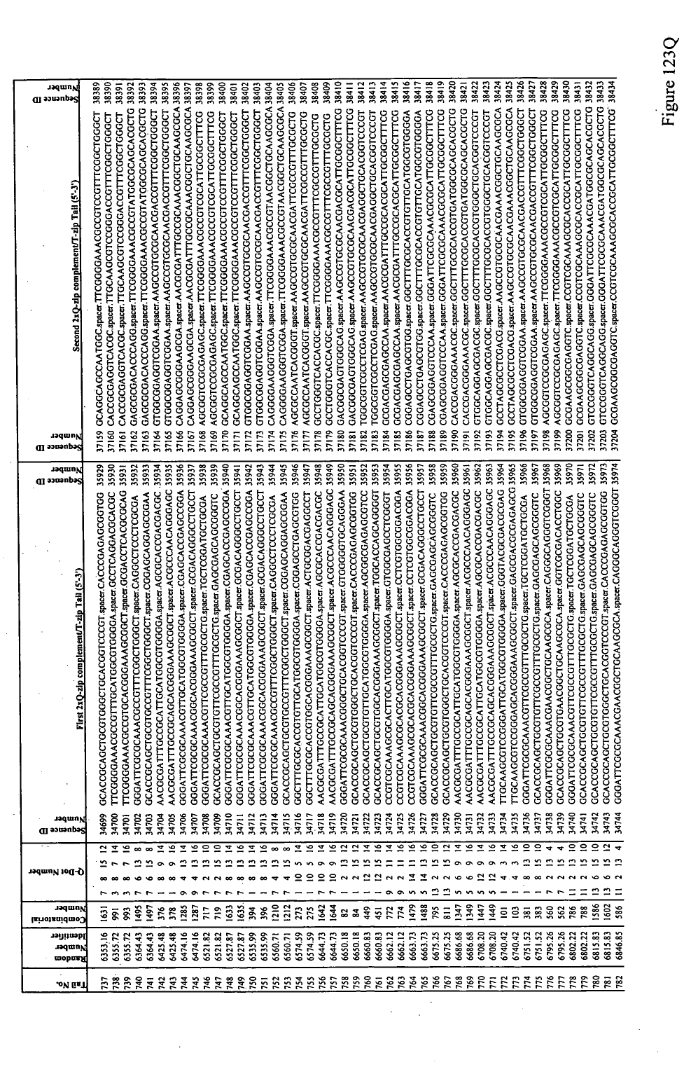

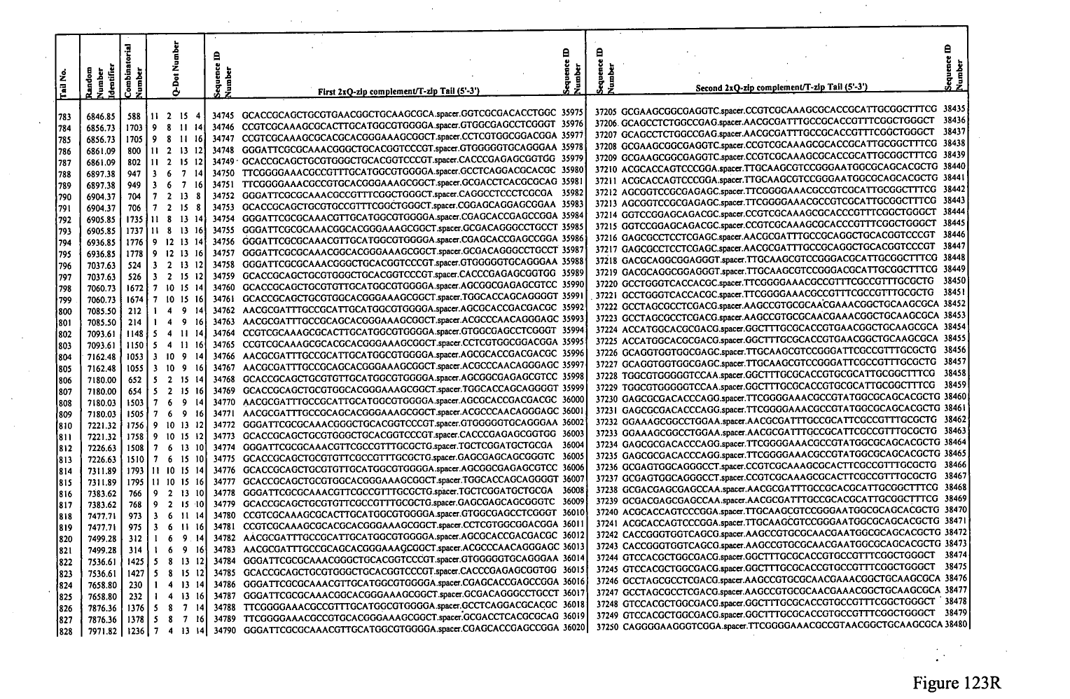

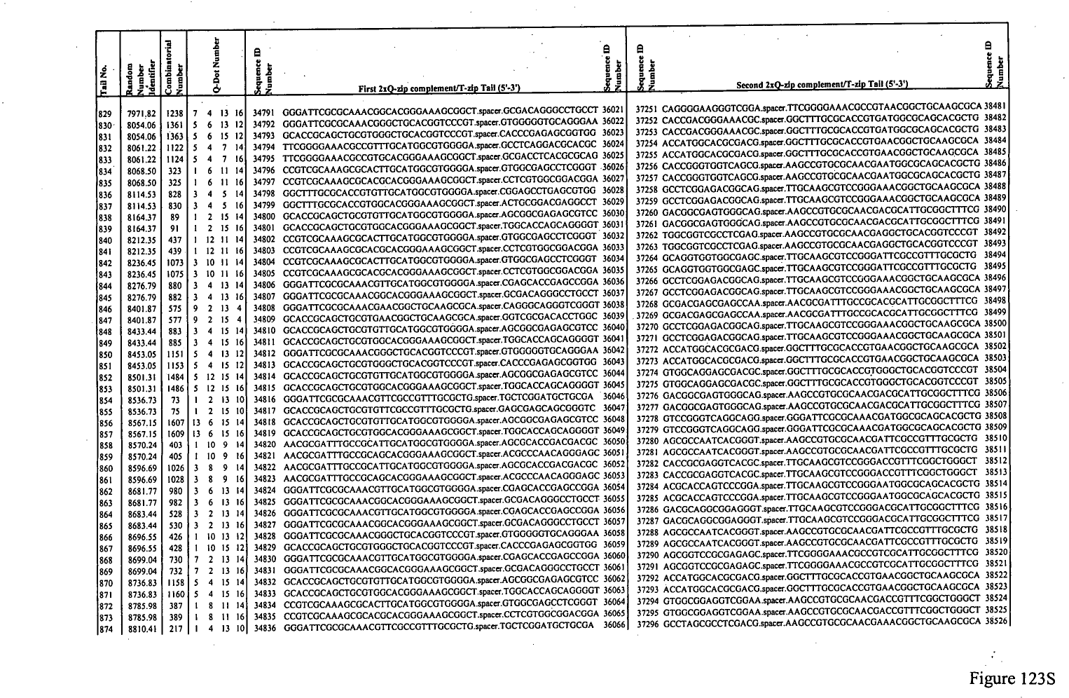

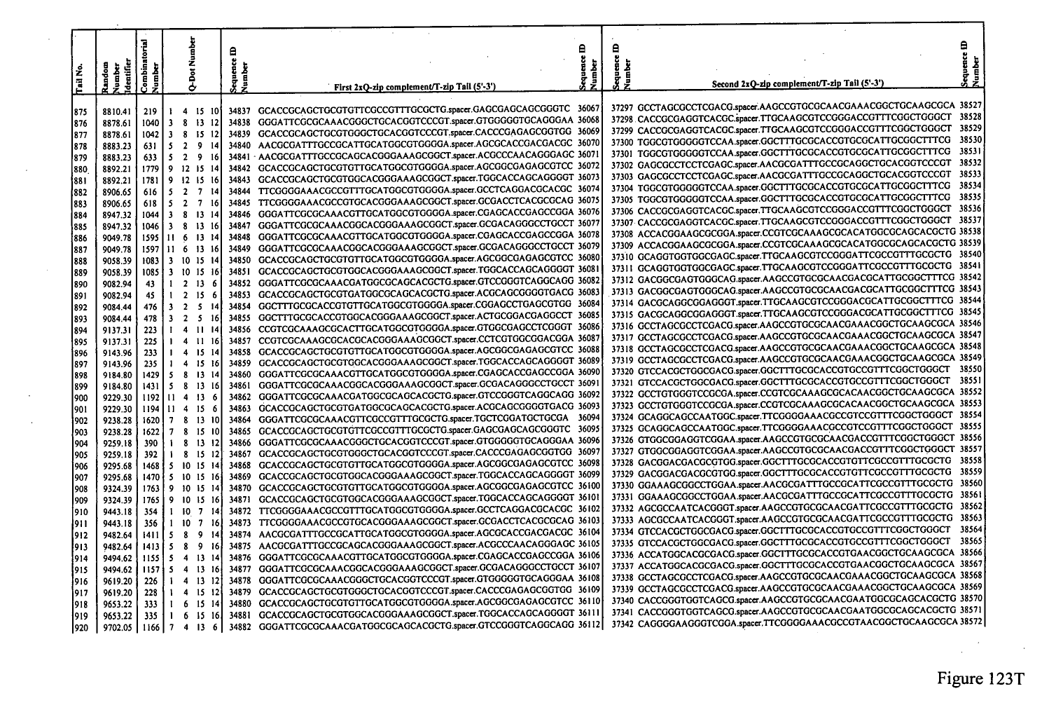

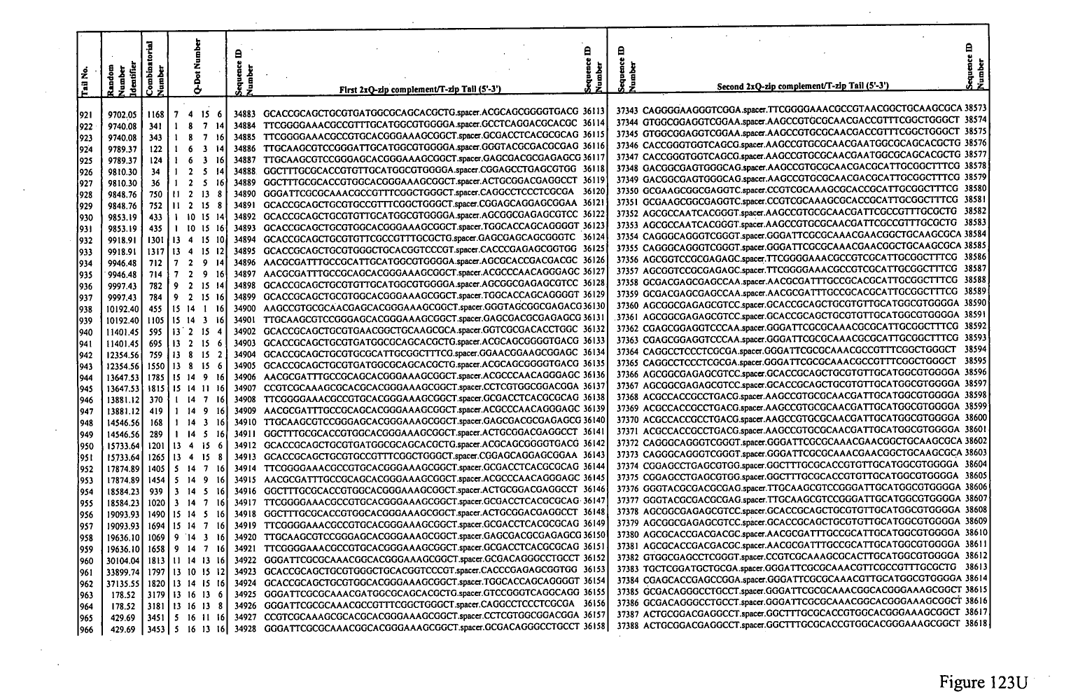

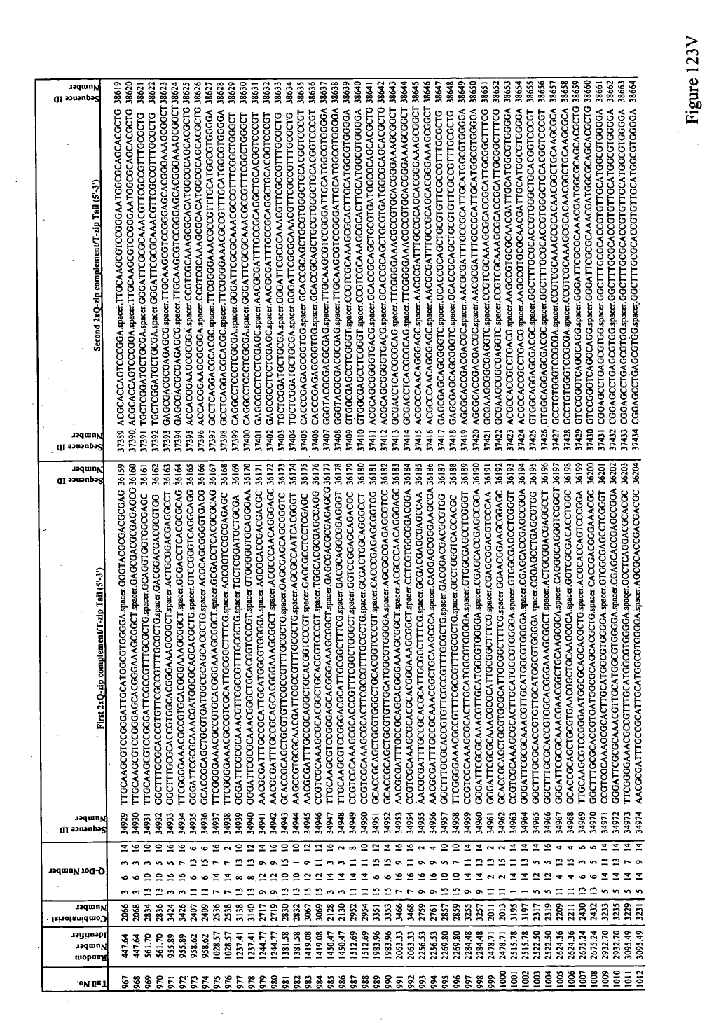

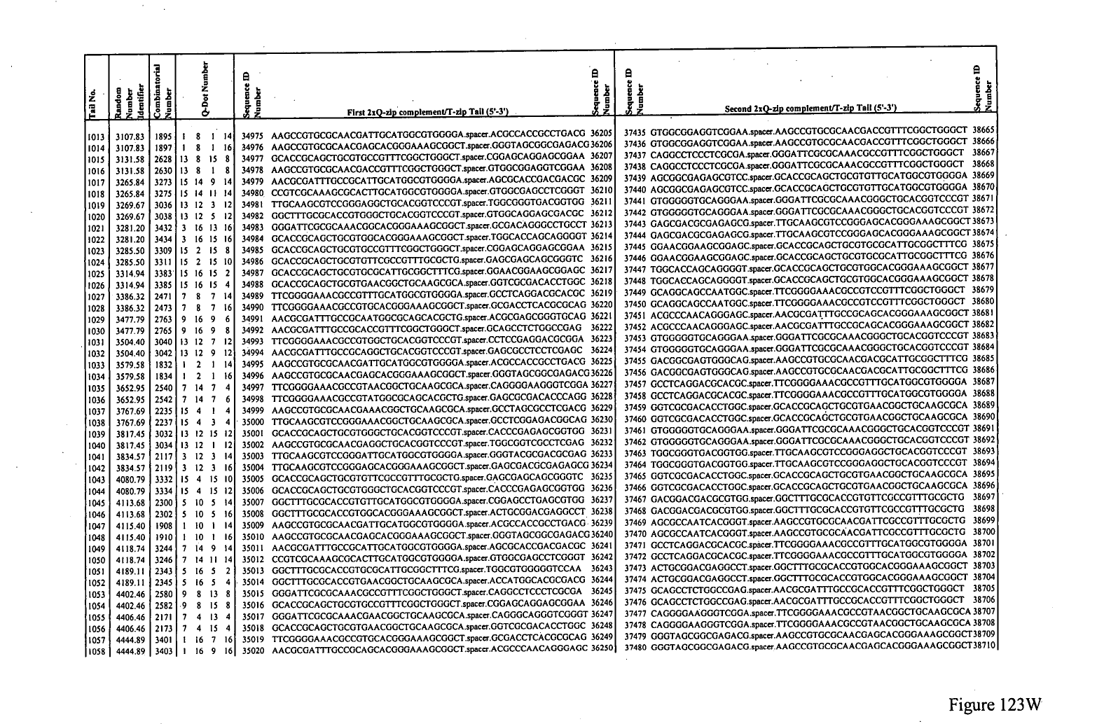

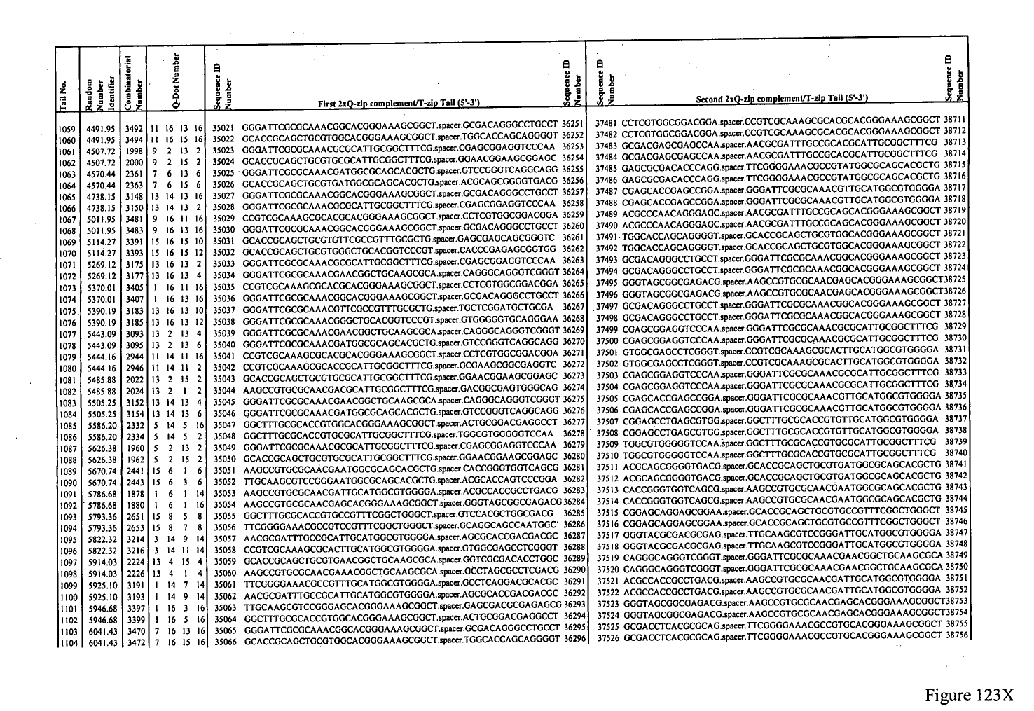

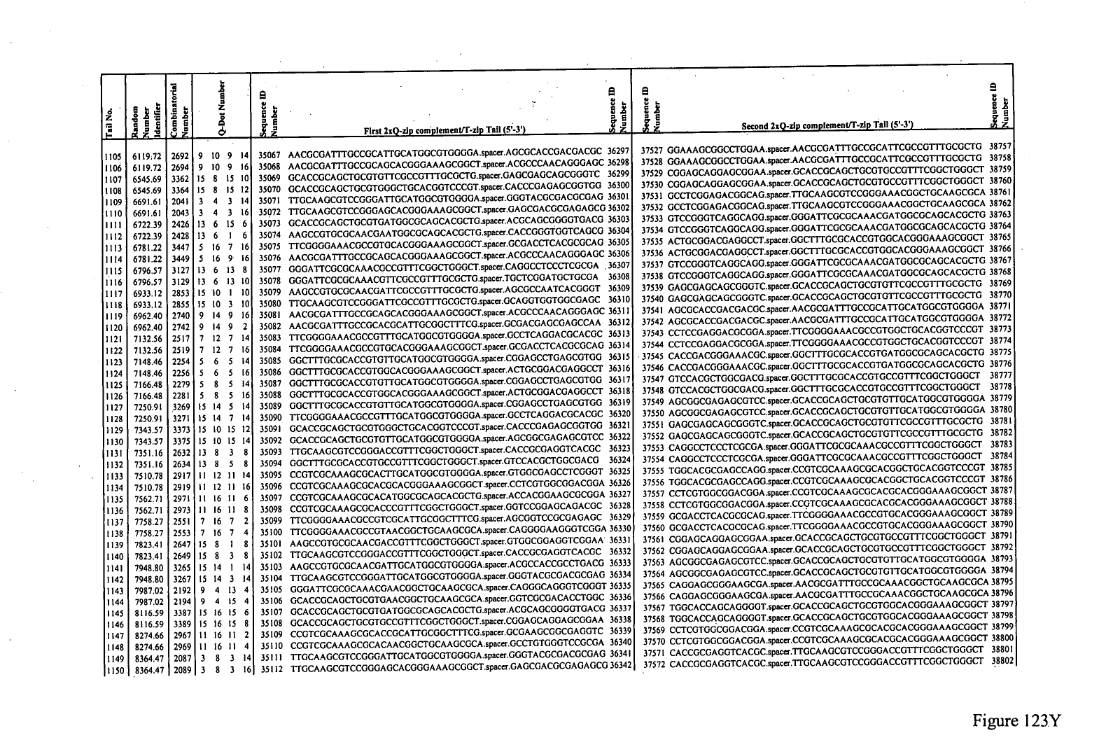

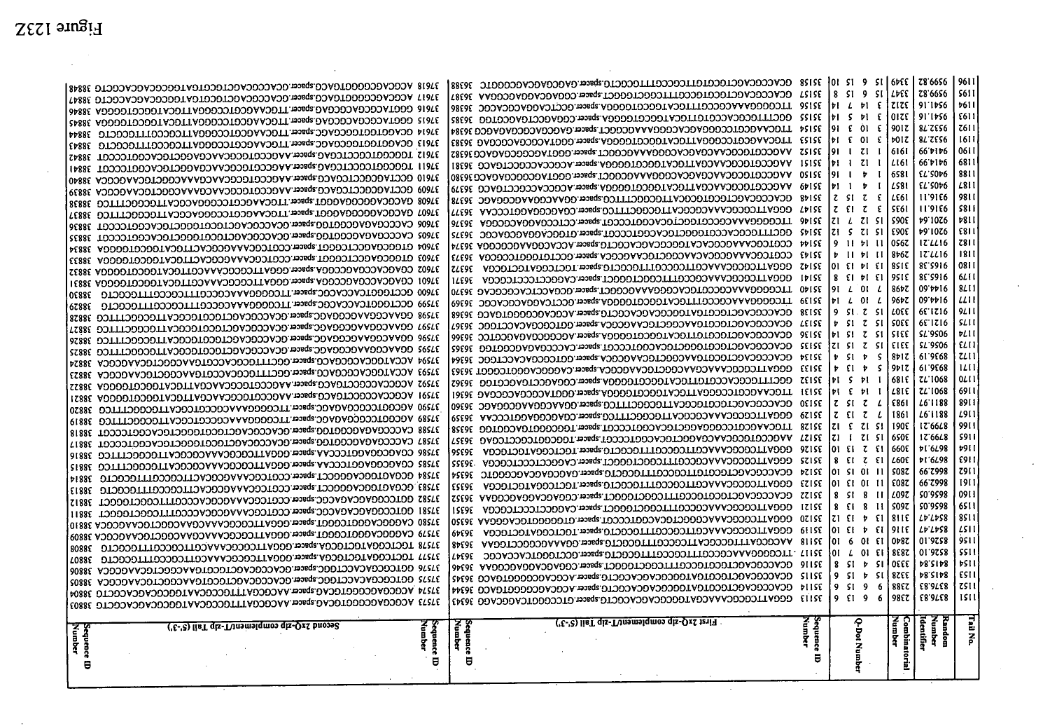

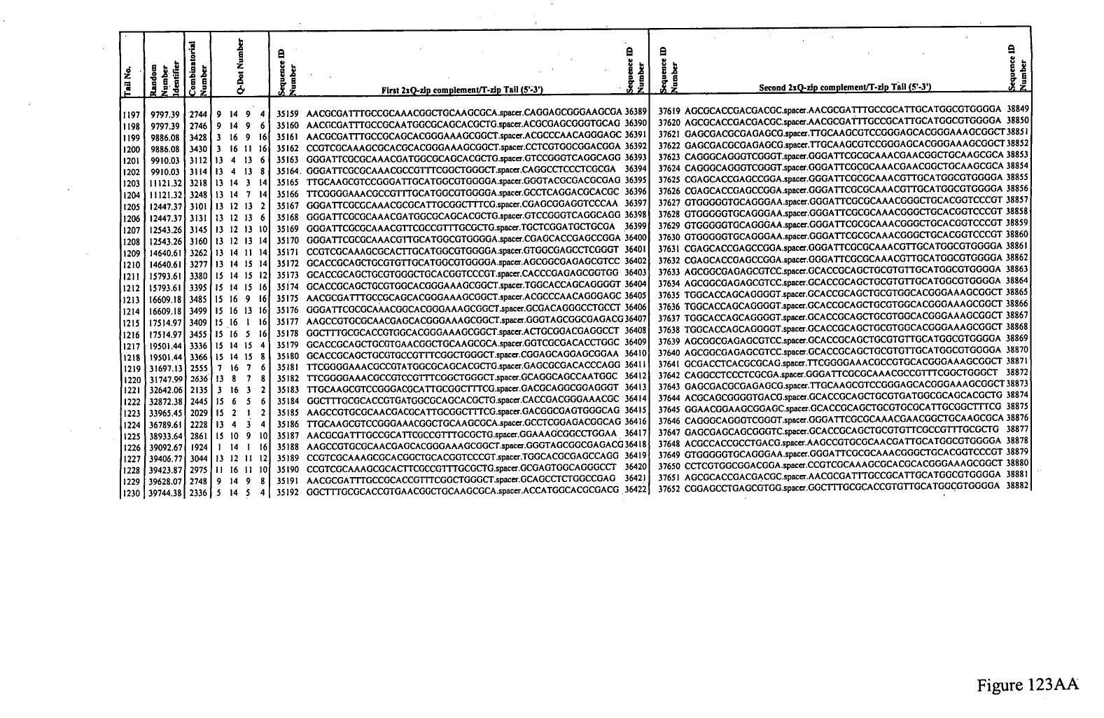

In one method, shown in FIG. 89A, Q-Zips bearing a 5′-biotin group or 5′-amino are then hybridized to the magnetic beads. The beads are captured and washed extensively to remove unhybridized oligonucleotides. Streptavidin Q-dots or COOH Q-dots with EDC are added to the beads to capture the Q-dots on to the Q-Zips and then washed extensively to remove unreacted Q-Zips. The product is denatured with 0.1 M NaOH to elute the Q-dots with monovalent tails.

In a second method, shown in FIG. 89B, streptavidin Q-dots or COOH Q-dots are instead treated with Q-Zips bearing a 5′-biotin or 5′-amino group and EDC respectively to conjugate the Q-dots to the Q-Zips. The oligonucleotides containing a capture group are hybridized to magnetic beads and washed extensively to remove unhybridzed oligonucleotides and unreacted Q-dots. The product is denatured with 0.1M NaOH to elute the Q-dots with monovalent tails.

FIG. 90 is a graph depicting the standard curve for Q-dot dilution.

FIG. 91 is a schematic diagram showing the effect of Q-dot loading on signal intensity.

FIG. 92 is a schematic diagram showing the titration of oligonucleotide probe concentration on magnetic microspheres using Biocytin.

FIG. 93 is a schematic diagram showing the titration of oligonucleotide probe concentration on magnetic microspheres using Zip1.

FIG. 94 is a schematic diagram showing an estimation of the relative intensity of Q-dot/Q-Zip1 and Q-dot/Q-Zip2 signals.

FIG. 95 is a schematic diagram showing indirect hybridization of Q-dots to magnetic microspheres using a ‘bridging oligo’.

FIG. 96 shows a list of 16 Q-Zip probes (SEQ ID NO:1 through SEQ ID NO:16), and their 16 complements (SEQ ID NO:17 through SEQ ID NO:32).

FIG. 97 shows a list of 16 complementary Q-Zip probe-containing tails (“Q-tails”) (SEQ ID NO:33 through SEQ ID NO:48). The tails are each 48mers and consist of three concatenated 16mer Q-Zip probes. The tails were constructed from the four complementary Q-Zip probes (SEQ ID NO:17 through SEQ ID NO:20) labeled QZ1-QZ4 in FIG. 96, as described in Example 2.

FIG. 98 shows a list of 16 Q-tails (SEQ ID NO:833 through SEQ ID NO:848). The tails are each 48mers and consist of three concatenated 16mer Q-Zip portions. The tails were constructed from the four Q-Zip probes (SEQ ID NO:1 through SEQ ID NO:4) labeled QZ1-QZ4 in FIG. 96.

FIGS. 99A-D show a list of 112 complementary Q-tails (SEQ ID NO:33 through SEQ ID NO:144). The tails are each 48mers and consist of three concatenated 16mer Q-Zip portions. The tails were constructed from the eight complementary Q-Zip portions (SEQ ID NO:17 through SEQ ID NO:24) labeled QZ1-QZ8 in FIG. 96, as described in Example 2.

FIGS 100A-D show a list of 112 Q-tails (SEQ ID NO:833 through SEQ ID NO:944). The tails are each 48mers and consist of three concatenated 16mer Q-Zip portions. The tails were constructed from the eight Q-Zip portions (SEQ ID NO:1 through SEQ ID NO:8) labeled QZ1-QZ8 in FIG. 96.

FIGS. 101A-M show a list of 352 complementary Q-tails (SEQ ID NO:33 through SEQ ID NO:384). The tails are each 48mers and consist of three concatenated 16mer Q-Zip portions. The tails were constructed from the twelve complementary Q-Zip portions (SEQ ID NO:17 through SEQ ID NO:28) labeled QZ1-QZ12 in FIG. 96.

FIGS. 102A-J show a list of 352 Q-tails (SEQ ID NO:833 through SEQ ID NO:1184). The tails are each 48mers and consist of three concatenated 16mer Q-Zip portions. The tails were constructed from the twelve Q-Zip portions (SEQ ID NO:1 through SEQ ID NO:12) labeled QZ1-QZ12 in FIG. 96.

FIGS. 103A-X show a list of 800 complementary Q-tails (SEQ ID NO:33 through SEQ ID NO:832). The tails are each 48mers and consist of three concatenated 16mer Q-Zip portions. The tails were constructed from the sixteen complementary Q-Zip portions (SEQ ID NO:17 through SEQ ID NO:32) labeled QZ1-QZ16 in FIG. 96.

FIGS. 104A-X show a list of 800 Q-tails (SEQ ID NO:833 through SEQ ID NO:1632). The tails are each 48mers and consist of three concatenated 16mer Q-Zip portions. The tails were constructed from the sixteen Q-Zip portions (SEQ ID NO:1 through SEQ ID NO:16) labeled QZ1-QZ12 in FIG. 96.

FIGS. 105A-G show a list of 238 complementary Q-tails (SEQ ID NO:1633 through SEQ ID NO:1870). The tails are each 64 mers and consist of four concatenated 16mer Q-Zip portions. The tails were constructed from the eight complementary Q-Zip portions (SEQ ID NO:17 through SEQ ID NO:24) labeled QZ1-QZ8 in FIG. 96.

FIGS. 106A-DD show a list of 1155 complementary Q-tails (SEQ ID NO:1633 through SEQ ID NO:2787). The tails are each 64 mers and consist of four concatenated 16mer Q-Zip portions. The tails were constructed, from the twelve complementary Q-Zip portions (SEQ ID NO:17 through SEQ ID NO:28) labeled QZ1-QZ12 in FIG. 96.

FIGS. 107A-TTTT show a list of 3500 complementary Q-tails (SEQ ID NO:1633 through SEQ ID NO:5132). The tails are each 64 mers and consist of four concatenated 16mer Q-Zip portions. The tails were constructed from the sixteen complementary Q-Zip portions (SEQ ID NO:17 through SEQ ID NO:32) labeled QZ1-QZ16 in FIG. 96.

FIGS. 108A-G show a list of 238 pairs of complementary Q-tails. Each tail (SEQ ID NO:7593 through SEQ ID NO:7830; SEQ ID NO:11093 through SEQ ID NO:1330) is a 32 mer and consists of two concatenated Q-Zip portions. The tails were constructed from the eight complementary Q-Zip portions (SEQ ID NO:17 through SEQ ID NO:24) labeled QZ1-QZ8 in FIG. 96. The first tail (i.e. the discriminating oligonucleotide probe) in each pair will ligate to the second tail (i.e. the common oligonucleotide probe) during the ligase detection reaction if the allele-specific oligonucleotide probe is present. Two Q-Zip portions containing Q-dots will hybridize with each oligonucleotide probe, allowing detection of four Q-dots per oligonucleotide probe.

FIG. 109A-Z show a list of 1155 pairs of complementary Q-tails. Each tail (SEQ ID NO:7593 through SEQ ID NO:8747; SEQ ID NO:11093 through SEQ ID NO:12247) is a 32 mer and consists of two concatenated Q-Zip portions. The tails were constructed from the twelve complementary Q-Zip portions (SEQ ID NO:17 through SEQ ID NO:28) labeled QZ1-QZ12 in FIG. 96. The first tail (i.e. the discriminating oligonucleotide probe) in each pair will ligate to the second tail (i.e. the common oligonucleotide probe) during the ligase detection reaction if the allele-specific oligonucleotide probe is present. Two Q-Zip portions containing Q-dots will hybridize with each oligonucleotide probe, allowing detection of four Q-dots per oligonucleotide probe.

FIGS. 110A-LLLL show a list of 3500 pairs of complementary Q-tails. Each tail (SEQ ID NO:7593 through SEQ ID NO:11092; SEQ ID NO:11093 through SEQ ID NO:14592) is a 32 mer and consists of two concatenated Q-Zip portions. The tails were constructed from the sixteen complementary Q-Zip portions (SEQ ID NO:17 through SEQ ID NO:32) labeled QZ1-QZ16 in FIG. 96. The first tail (i.e. the discriminating oligonucleotide probe) in each pair will ligate to the second tail (i.e. the common oligonucleotide probe) during the ligase detection reaction if the allele-specific oligonucleotide is present. Two Q-Zip portions containing Q-dots will hybridize with each oligonucleotide probe, allowing detection of four Q-dots per oligonucleotide probe.

FIGS. 111A-B show a list of 84 pairs of complementary Q-tails. Each tail (SEQ ID NO:5133 through SEQ ID NO:5216; SEQ ID NO:6363 through SEQ ID NO:6446) is a 32 mer and consists of two concatenated Q-Zip portions. The tails were constructed from the eight complementary Q-Zip portions (SEQ ID NO:17 through SEQ ID NO:24) labeled QZ1-QZ8 in FIG. 96. The first tail (i.e. the discriminating oligonucleotide probe) in each pair will ligate to the second tail (i.e. the common oligonucleotide probe) during the ligase detection reaction if the allele-specific oligonucleotide probe is present. Two Q-Zip portions containing Q-dots will hybridize with each oligonucleotide probe, allowing detection of four Q-dot probes per oligonucleotide probe. The two Q-Zip portions containing Q-dots hybridizing to the same oligonucleotide probe will also ligate to each other in the presence of thermostable ligase.

FIGS. 112A-I show a list of 403 pairs of complementary Q-tails. Each tail (SEQ ID NO:5133 through SEQ ID NO:5535; SEQ ID NO:6363 through SEQ ID NO:6765) is a 32 mer and consists of two concatenated Q-Zip portions. The tails were constructed from the twelve complementary Q-Zip portions (SEQ ID NO:17 through SEQ ID NO:28) labeled QZ1-QZ12 in FIG. 96. The first tail (i.e. the discriminating oligonucleotide probe) in each pair will ligate to the second tail (i.e. the common oligonucleotide probe) during the ligase detection reaction if the allele-specific oligonucleotide is present. Two Q-Zip portions containing Q-dots will hybridize with each oligonucleotide probe, allowing detection of four Q-Zip portions containing Q-dots per oligonucleotide probe. The two Q-Zip portions containing Q-dots hybridizing to the same oligonucleotide probe will also ligate to each other in the presence of thermostable ligase.

FIGS. 113A-BB show a list of 1230 pairs of complementary Q-tails. Each tail (SEQ ID NO:5133 through SEQ ID NO:6362; SEQ ID NO:6363 through SEQ ID NO:7592) is a 32 mer and consists of two concatenated Q-Zip portions. The tails were constructed from the sixteen complementary Q-Zip portions (SEQ ID NO:17 through SEQ ID NO:32) labeled QZ1-QZ16 in FIG. 96. The first tail (i.e. the discriminating oligonucleotide probe) in each pair will ligate to the second tail (i.e. the common oligonucleotide probe) during the ligase detection reaction if the allele-specific oligonucleotide is present. Two Q-Zip portions containing Q-dots will hybridize with each oligonucleotide probe, allowing detection of four Q-dots per oligonucleotide probe. The two Q-Zip portions containing Q-dots hybridizing to the same oligonucleotide probe will also ligate to each other in the presence of thermostable ligase.

FIGS. 114A-D show a list of 120 single complementary Q-tails. Each tail (SEQ ID NO:14593 through SEQ ID NO:14712) is a 32 mer and consists of two concatenated Q-Zip portions. The tails were constructed from the sixteen complementary Q-Zip portions (SEQ ID NO:17 through SEQ ID NO:32) labeled QZ1-QZ16 in FIG. 96. Two Q-Zip portions containing Q-dots will hybridize with each tail, allowing detection of two Q-dots per oligonucleotide probe.

FIGS. 115A-E show a list of 120 single Q-tails. Each tail (SEQ ID NO:14713 through SEQ ID NO:14832) is a 32 mer and consists of two concatenated Q-Zip portions. The tails were constructed from the sixteen Q-Zip portions (SEQ ID NO:1 through SEQ ID NO:16) labeled QZ1-QZ16 in FIG. 96. Two Q-Zip portions containing Q-dots will hybridize with each tail, allowing detection of two Q-dots per oligonucleotide probe.

FIGS. 116A-C show a list of 64 single complementary Q-tails. Each tail (SEQ ID NO:14833 through SEQ ID NO:14896) is a 32 mer and consists of two concatenated Q-Zip portions. The tails were constructed from the sixteen complementary Q-Zip portions (SEQ ID NO:17 through SEQ ID NO:32) labeled QZ1-QZ16 in FIG. 96. Two Q-Zip portions containing Q-dots will hybridize with each tail, allowing detection of two Q-dot probes per oligonucleotide probe. Upon hybridization, Q-Zip portions containing Q-dots will ligate to each other in the presence of thermostable ligase.

FIGS. 117A-C show a list of 64 single Q-tails. Each tail (SEQ ID NO:14897 through SEQ ID NO:14960) is a 32 mer and consists of two concatenated Q-Zip portions. The tails were constructed from the sixteen Q-Zip portions (SEQ ID NO:1 through SEQ ID NO:16) labeled QZ1-QZ16 in FIG. 96. Two Q-Zip portions containing Q-dots will hybridize with each tail, allowing detection of two Q-dot probes per oligonucleotide probe. Upon hybridization, Q-Zip portions containing Q-dots will ligate to each other in the presence of thermostable ligase.

FIGS. 118A-E show a list of 136 T-Zip portions (SEQ ID NO:14961 through SEQ ID NO:15096), and their 136 complements (SEQ ID NO:15097 through SEQ ID NO:15232). The T-Zip portions are each 16mers and were constructed.

FIGS. 119A-E show the list of 136 T-Zip portions (SEQ ID NO:14961 through SEQ ID NO:15096) and their 136 complements (SEQ ID NO:15097 through SEQ ID NO:15232) shown in FIG. 118. Also shown are the Q-Zip portions and reverse Q-Zip portion numbers.

FIGS. 120A-UUUUU show a list of 3500 T-Zip portion-containing tails (“T-Zip tails”) (SEQ ID NO:15233 through SEQ ID NO:18732) (initial LDR oligonucleotide probes). Each tail is a 32 mer and consists of two concatenated T-Zip portions. The tails were constructed from the 136 T-Zip portions (SEQ ID NO:14961 through SEQ ID NO:15096) shown in FIG. 119.

FIGS. 121A-YYY show a list of 3500 upstream and 3500 downstream oligonucleotide probes. Each upstream oligonucleotide probe consists of two concatenated Q-Zip portions followed by a spacer and a T-Zip portion. Each downstream oligonucleotide probe consists of one T-Zip portion followed by a spacer and two concatenated complementary Q-Zip portions. The oligonucleotide probes were constructed from the 136 T-Zip portions (SEQ ID NO:14961 through SEQ ID NO:15096) shown in FIG. 119 and the 16 complementary Q-Zip portions (SEQ ID NO:17 through SEQ ID NO:32) labeled QZ1-QZ16 in FIG. 96. Sequence identification numbers refer to the nucleotide sequence immediately following or immediately preceding the sequence identification number, as applicable.

FIGS. 122A-RR show a list of 1230 T-Zip tails (SEQ ID NO:18733 through SEQ ID NO:19962) (initial LDR oligonucleotide probes). Each tail is a 32 mer and consists of two concatenated T-Zip portions. The tails were constructed from the 136 T-Zip portions (SEQ ID NO:14961 through SEQ ID NO:15096) shown in FIG. 119.

FIGS. 123A-AA show a list of 1230 upstream and 1230 downstream oligonucleotide probes. Each upstream oligonucleotide probe consists of one ligated Q-tail followed by a spacer and a T-Zip portion. Each downstream oligonucleotide probe consists of one T-Zip portion followed by a spacer and one complementary ligated Q-tail. The oligonucleotide probes were constructed from the 1230 T-Zip tails (SEQ ID NO:18733 through SEQ ID NO:19962) shown in FIG. 122, and the 1230 Q-tails (SEQ ID NO:5133 through SEQ ID NO:6362) and 1230 complementary Q-tails (SEQ ID NO:6363 through SEQ ID NO:7592) shown in FIG. 113. Sequence identification numbers refer to the nucleotide sequence immediately following or immediately preceding the sequence identification number, as applicable.

### DETAILED DESCRIPTION OF THE INVENTION

Ligase Detection Reaction/Detection Probe Hybridization/Detection Scheme

The present invention relates to a method for identifying one or more target nucleic acid molecules differing by one or more single-base changes, insertions, deletions, or translocations. This method includes a ligase detection reaction phase, a detection probe hybridization phase, and a detection phase.

The ligation detection reaction phase involves providing a test sample potentially containing one or more target nucleic acid molecules differing by one or more single-base changes, insertions, deletions, or translocations. One or more primary oligonucleotide probe sets are used in this phase. Each set is characterized by (a) a first oligonucleotide probe, having a target-specific portion and (b) a second oligonucleotide probe, having a target-specific portion, where the oligonucleotide probes in a particular set are suitable for ligation together when hybridized on a corresponding target nucleic acid molecule, but have a mismatch which interferes with such ligation when hybridized to any other nucleic acid molecule present in the sample. One or both oligonucleotide probes in the set contain one or more detection oligonucleotide probe-specific portions or their complements such that each probe set contains a unique set of one or more detection oligonucleotide probe-specific portions or their complements. The sample, the one or more primary oligonucleotide probe sets, and a ligase are blended to form a primary ligase detection reaction mixture. The primary ligase detection reaction mixture is subjected to one or more ligase detection reaction cycles. These cycles each include a denaturation treatment and a hybridization treatment. In the denaturation treatment, any hybridized oligonucleotides are separated from the target nucleic acid molecules. With the hybridization treatment, the primary oligonucleotide probe sets hybridize in a base-specific manner to their respective target nucleic acid molecules, if present in the sample, and ligate to one another to form a primary ligation product containing the target-specific portions and one or more detection oligonucleotide probe-specific portions or their complements. The primary ligation product for each of the primary oligonucleotide probe sets are distinguishable from other nucleic acid molecules in the primary ligase detection reaction mixture by a unique set of one or more detection oligonucleotide probe-specific portions or their complements. The primary oligonucleotide probe sets may hybridize to nucleic acid molecules in the sample other than their respective target nucleic acid molecules but do not ligate together due to the presence of one or more mismatches and individually separate during the denaturation treatment.

For the detection probe hybridization phase, detection oligonucleotide probes which bind to the complementary detection oligonucleotide probe-specific portion of the captured primary ligation product or complements thereof are provided. Each detection oligonucleotide probe has a reporter label, thereby providing each primary ligation product with a unique detectable encryption code. The primary ligation products and the detection oligonucleotide probes are contacted under conditions effective to permit hybridization of the detection oligonucleotide probes to the primary ligation products so that a labeled primary ligation product is formed.

In the detection phase, the reporter label(s) on the primary ligation product are detected, thereby indicating the presence of one or more target nucleic acid molecules in the sample. Nucleic acid molecules differing by one or more single-base changes, insertions, deletions, or translocations are discriminated from one another during the one or more ligase detection reaction cycles and the discriminated nucleic acid molecules are detected as a result of each different labeled, primary ligation product having a unique encryption code with a different pattern of detectable emission spectra.

The ligase detection reaction process, in accordance with the present invention, is described generally in WO 90/17239 to Barany et al., F. Barany et al., “Cloning, Overexpression and Nucleotide Sequence of a Thermostable DNA Ligase-encoding Gene,” Gene 109:1-11 (1991), and F. Barany, “Genetic Disease Detection and DNA Amplification Using Cloned Thermostable Ligase,” Proc. Natl. Acad. Sci. USA, 88:189-193 (1991), the disclosures of which are hereby incorporated by reference in their entirety. In accordance with the present invention, the ligase detection reaction can use 2 sets of complementary oligonucleotides. This is known as the ligase chain reaction which is described in the 3 immediately preceding references, which are hereby incorporated by reference in their entirety. Alternatively, the ligase detection reaction can involve a single cycle which is known as the oligonucleotide ligation assay. See Landegren, et al., “A Ligase-Mediated Gene Detection Technique,” Science 241:1077-80 (1988); Landegren, et al., “DNA Diagnostics—Molecular Techniques and Automation,” Science 242:229-37 (1988); and U.S. Pat. No. 4,988,617 to Landegren, et al.

During the ligase detection reaction phase of the process, the denaturation treatment is carried out at a temperature of 80-105° C., while hybridization takes place at 50-85° C. Each cycle comprises a denaturation treatment and a thermal hybridization treatment which in total is from about one to five minutes long. Typically, the ligation detection reaction involves repeatedly denaturing and hybridizing for 2 to 50 cycles. The total time for the ligase detection reaction phase of the process is 1 to 250 minutes.

The oligonucleotide probe sets can be in the form of ribonucleotides, deoxynucleotides, modified ribonucleotides, modified deoxyribonucleotides, peptide nucleotide analogues, modified peptide nucleic acid analogues, modified phosphate-sugar-backbone oligonucleotides, nucleotide analogs, and mixtures thereof.

In one variation, the oligonucleotides of the oligonucleotide probe sets each have a hybridization or melting temperature (i.e. Tₘ) of 66-70° C. These oligonucleotides are 20-28 nucleotides long.

It may be desirable to destroy chemically or enzymatically unconverted LDR oligonucleotide probes that contain addressable nucleotide array-specific portions after the ligase detection reaction process is completed. Such unconverted probes will otherwise compete with ligation products for hybridization to other nucleic acid molecules during downstream processing. Destruction can be accomplished by utilizing an exonuclease, such as exonuclease III (L-H Guo and R. Wu, Methods in Enzymology 100:60-96 (1985), which is hereby incorporated by reference in its entirety) in combination with LDR probes that are blocked at the ends and not involved with ligation of probes to one another. The blocking moiety could be a reporter group or a phosphorothioate group. T. T. Nikiforow, et al., “The Use of Phosphorothioate Primers and Exonuclease Hydrolysis for the Preparation of Single-stranded PCR Products and their Detection by Solid-phase Hybridization,” PCR Methods and Applications, 3:p. 285-291 (1994), which is hereby incorporated by reference in its entirety. After the LDR process, unligated probes are selectively destroyed by incubation of the reaction mixture with the exonuclease. The ligated probes are protected due to the elimination of free 3′ ends which are required for initiation of the exonuclease reaction. This approach results in an increase in the signal-to-noise ratio, especially where the LDR reaction forms only a small amount of product. Since unligated oligonucleotides compete for hybridization to other nucleic acid molecules in downstream processing, such competition with the ligated oligonucleotides lowers the signal. An additional advantage of this approach is that unhybridized label-containing sequences are degraded and, therefore, are less able to cause a target-independent background signal, because they can be removed more easily by washing.

Prior to the ligation detection reaction phase of the present invention, the sample is preferably amplified by an initial target nucleic acid amplification procedure. This increases the quantity of the target nucleotide sequence in the sample. For example, the initial target nucleic acid amplification may be accomplished using the polymerase chain reaction process, self-sustained sequence replication, Q-β replicase-mediated RNA amplification, or ligase chain reaction. The polymerase chain reaction process is the preferred amplification procedure and is fully described in H. Erlich, et. al., “Recent Advances in the Polymerase Chain Reaction,” Science 252: 1643-50 (1991); M. Innis, et. al., PCR Protocols: A Guide to Methods and Applications, Academic Press: New York (1990); and R. Saiki, et. al., “Primer-directed Enzymatic Amplification of DNA with a Thermostable DNA Polymerase,” Science 239: 487-91 (1988), which are hereby incorporated by reference in their entirety. The ligase chain reaction process is fully described in F. Barany, et. al., “Cloning, Overexpression and Nucleotide Sequence of a Thermostable DNA Ligase-encoding Gene,” Gene 109: 1-11 (1991) and F. Barany, “Genetic Disease Detection and DNA Amplification Using Cloned Thermostable Ligase,” Proc. Natl. Acad. Sci. USA 88: 189-93 (1991), which are hereby incorporated by reference in their entirety. J. Guatelli, et. al., “Isothermal, in vitro Amplification of Nucleic Acids by a Multienzyme Reaction Modeled After Retroviral Replication,” Proc. Natl. Acad. Sci. USA 87: 1874-78 (1990), which is hereby incorporated by reference in its entirety, describes the self-sustained sequence replication process. The Q-β replicase-mediated RNA amplification is disclosed in F. Kramer, et. al., “Replicatable RNA Reporters,” Nature 339: 401-02 (1989), which is hereby incorporated by reference in its entirety.

Of these amplification procedures, the polymerase chain reaction is preferred. In carrying out this procedure, the target nucleic acid, when present in the form of a double stranded DNA molecule is denatured to separate the strands. This is achieved by heating to a temperature of 80-105° C. Polymerase chain reaction primers are then added and allowed to hybridize to the strands, typically at a temperature of 20-85° C. A thermostable polymerase (e.g., Thermus aquaticus polymerase) is also added, and the temperature is then adjusted to 50-85° C. to extend the primer along the length of the nucleic acid to which the primer is hybridized. After the extension phase of the polymerase chain reaction, the resulting double stranded molecule is heated to a temperature of 80-105° C. to denature the molecule and to separate the strands. These hybridization, extension, and denaturation steps may be repeated a number of times to amplify the target nucleic acid to an appropriate level.

In carrying out the ligase detection reaction of the present invention, a suitable thermostable ligase can be isolated from Thermus aquaticus, as set forth in M. Takahashi, et al., “Thermophillic DNA Ligase,” J. Biol. Chem. 259:10041-47 (1984), which is hereby incorporated by reference in its entirety. Alternatively, it can be prepared recombinantly. Procedures for such isolation as well as the recombinant production of Thermus aquaticus ligase (and Thermus themophilus ligase) are disclosed in WO 90/17239 to Barany, et. al., and F. Barany, et al., “Cloning, Overexpression and Nucleotide Sequence of a Thermostable DNA-Ligase Encoding Gene,” Gene 109:1-11 (1991), which are hereby incorporated by reference in their entirety. These references contain complete sequence information for this ligase as well as the encoding DNA. Other suitable ligases include, without limitation, E. coli ligase, T4 ligase, Thermus sp. AK16 ligase (WO 00/26381 to Barany et al., which is hereby incorporated by reference), Aquifex aeolicus ligase, Thermotoga maritima ligase, and Pyrococcus ligase. The ligation detection reaction mixture may include a carrier DNA, such as salmon sperm DNA.

The hybridization step, which is preferably a thermal hybridization treatment, discriminates between nucleotide sequences based on a distinguishing nucleotide at the ligation junctions. The difference between the target nucleotide sequences can be, for example, a single nucleic acid base difference, a nucleic acid deletion, a nucleic acid insertion, or translocation. Such sequence differences involving more than one base can also be detected. Preferably, the oligonucleotide probe sets have substantially the same length so that they hybridize to target nucleotide sequences at substantially similar hybridization conditions. As a result, the process of the present invention is able to detect infectious diseases, genetic diseases, and cancer. It is also useful in environmental monitoring, forensics, and food science.

A wide variety of infectious diseases can be detected by the process of the present invention. Typically, these are caused by bacterial, viral, parasite, and fungal infectious agents. The resistance of various infectious agents to drugs can also be determined using the present invention.

Bacterial infectious agents which can be detected by the present invention include, without limitation, Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Actinomycetes.

Fungal infectious agents which can be detected by the present invention include, without limitation, Cryptococcus neoformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis.

Viral infectious agents which can be detected by the present invention include, without limitation, human immunodeficiency virus, human T-cell lymphocytotrophic virus, hepatitis viruses (e.g., Hepatitis B Virus and Hepatitis C Virus), Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

Parasitic agents which can be detected by the present invention include, without limitation, Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

The present invention is also useful for detection of drug resistance by infectious agents. For example, vancomycin-resistant Enterococcus faecium, methicillin-resistant Staphylococcus aureus, penicillin-resistant Streptococcus pneumoniae, multi-drug resistant Mycobacterium tuberculosis, and AZT-resistant human immunodeficiency virus can all be identified with the present invention.

Genetic diseases can also be detected by the process of the present invention. This can be carried out by prenatal screening for chromosomal and genetic aberrations or post natal screening for genetic diseases. Examples of detectable genetic diseases include, without limitation, 21 hydroxylase deficiency, cystic fibrosis, Fragile X Syndrome, Turner Syndrome, Duchenne Muscular Dystrophy, Down Syndrome or other trisomies, heart disease, single gene diseases, HLA typing, phenylketonuria, sickle cell anemia, Tay-Sachs Syndrome, thalassemia, Kinefelter's Syndrome, Huntington's Disease, autoimmune diseases, lipidosis, obesity defects, hemophilia, inborn errors in metabolism, and diabetes.

Cancers which can be detected by the process of the present invention generally involve oncogenes, tumor suppressor genes, or genes involved in DNA amplification, replication, recombination, or repair. Examples of these include, without limitation, MSH2 gene, MLH2 gene, APC, AKT, ALT, AXL, BAX, Bcl2, Beta-Catenin, bFGF, BRCA1, BRCA2, Braf, Cdc25A, Cdk4, c-Fos, c-Jun, c-Kit, C-met, c-Myc, c-Ret, CSF1R, CSF2, c-Src, CYCD-CDK4, CYCE-CDK2, Cyclin D1, Cyclin E1, Cytokines, Dishevelled, E2F, E-Cadherin, EGFR, elF4E, ErbB-3, ErbB-4, FGFR-1, FGFR-2, FGFR-3, FGFR-4, FH4 (VEGFR-3), Fit-1 (VEGFR-1), Flk-1 (VEGFR-2), Frizzled, G Proteins, GPCR, GRB2-SOS, GSK3 beta, Her2-neu, HGF, HSP27, HSP70, IFGII, IGFR1, K-ras, H-ras, N-ras, LT, MAPK, MDM2, MEK, MLH1, MSH2, MSH6, MYC, p15^{INK4b}, p16^{INK4a}, p19^{ARF}, p21^{Cip}, p27^{Kip}, p53, PDGFR alpha, PDGFR beta, PI3K, PP2A, PTEN, RAF, RAS, RB, Ron, RSK, RTK, Ski, Smad2, Smad4, ST, surviving, TbRII, TCF, Tcf4, TERT, TGF-Beta, TGF-Beta R, TIC2, TOR, VEGF, WAF1, Wisp-1, Wisp-3, WNT, SNPs within cancer genes or adjacent regions that serve as markers for copy number changes or loss of heterozygosity in such genes, BRCA1 gene, p53 gene, Familial polyposis coli, Her2/Neu amplification, Bcr/Ab1, K-ras gene, human papillomavirus Types 16 and 18, leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, ENT tumors, and loss of heterozygosity. Suitable adjacent regions can be in areas in the 100 kilobases adjacent to known or candidate cancer genes. An example of this is SNPs adjacent to Her2-Neu that co-amplifies Her-Neu gene or SNPs or the Q-arm of chromosome 18 that may used to score loss of heterozygosity on 18Q.

In the area of environmental monitoring, the present invention can be used, for example, for detection, identification, and monitoring of pathogenic and indigenous microorganisms in natural and engineered ecosystems and microcosms such as in municipal waste water purification systems and water reservoirs or in polluted areas undergoing bioremediation. It is also possible to detect plasmids containing genes that can metabolize xenobiotics, to monitor specific target microorganisms in population dynamic studies, or either to detect, identify, or monitor genetically modified microorganisms in the environment and in industrial plants.

The present invention can also be used in a variety or forensic areas, including, without limitation, for human identification for military personnel and criminal investigation, paternity testing and family relation analysis, HLA compatibility typing, and screening blood, sperm, or transplantation organs for contamination.

In the food and feed industry, the present invention has a wide variety of applications. For example, it can be used for identification and characterization of production organisms such as yeast for production of beer, wine, cheese, yogurt, bread, etc. Another area of use is with regard to quality control and certification of products and processes (e.g., livestock, pasteurization, and meat processing) for contaminants. Other uses include the characterization of plants, bulbs, and seeds for breeding purposes, identification of the presence of plant-specific pathogens, and detection and identification of veterinary infections.

Once the ligation detection reaction phase of the process is completed, the detection probe hybridization phase is initiated. During this phase of the process, the ligation products and the detection oligonucleotide probes are contacted with each other at a temperature of 25-90° C., preferably 60-80° C., and for a time period of 10-180 minutes, preferably up to 60 minutes.

The detection phase of the present invention may involve scanning and identifying if ligation of particular oligonucleotide sets occurred and correlating ligation to the presence or absence of the target nucleotide sequence in the test sample. Scanning can be carried out by scanning electron microscopy, confocal microscopy, charge-coupled device, scanning tunneling electron microscopy, infrared microscopy, atomic force microscopy, electrical conductance, and fluorescent or phosphor imaging. Correlating is carried out with a computer.

Suitable reporter labels in accordance with the present invention include, without limitation, nanocrystals. When nanocrystals are used as reporting labels in the present invention, detecting includes exciting the nanocrystals to produce an emission spectrum and evaluating the emission spectra of the nanocrystals.

The preferred embodiment of the present invention uses nanocrystals known as Quantum dots. Quantum dots (Q-dots) are nanometer sized semiconductor crystals with optical properties that are strongly dependent on both the size and the material of the crystal (Alivisatos, “Semiconductor Clusters, Nanocrystals, and Quantum Dots,” Science 271:933-937 (1996), which is hereby incorporated by reference in its entirety). Most notably, the absorption and emission spectra from Q-dots can be tuned across a broad range of the electromagnetic spectrum simply by changing their size (Bruchez et al., “Semiconductor Nanocrystals as Fluorescent Biological Labels,” Science 281:2013 (1998), which is hereby incorporated by reference in its entirety).

The principle behind the size dependent properties of Quantum dots is called “quantum confinement.” The absorbing and emitting states shift to higher energy as the size of the particle decreases, requiring more energy to “confine” the excitation within the crystal structure (Efros, et al., “Interband Absorption of Light in a Semiconductor Sphere,” Soviet Physics Semiconductors 16(7):772-775 (1982), which is hereby incorporated by reference in its entirety). 12 to 16 or more easily distinguishable Quantum dots may be synthesized in the visible and IR regions alone.

Quantum dots can be synthesized with a predetermined emission wavelength with an accuracy of ±2 nm, a full width at half-maximum (FWHM) less than 35 nm and quantum yields (QYs) as high as 50% (Hines et al., “Synthesis and Characterization of Strongly Luminescing Layered Semiconductor Nanoclusters ((CdSe)ZnS),” J. Phys. Chem. 100(2):468-471 (1996), Peng, et al., “Epitaxial Growth of Highly Luminescent CdSe/CdS Core/Shell Nanocrystals with Photostability and Electronic Accessibility,” J. Am. Chem. Soc. 119(30):7019-7029 (1997), Dabbousi et al., “(CdSe)ZnS Core-Shell Quantum Dots: Synthesis and Characterization of a Size Series of Highly luminescent Nanocrystallites,” J. Phys. Chem. B 101:9463 (1997), which is hereby incorporated by reference in their entirety). A recent advance in Q-dot synthesis was the discovery of “core/shell” Q-dots. These are Quantum dots made from one material (e.g. CdSe) that are coated with a shell of a second, higher bandgap material (e.g. ZnS). The shell protects the fluorescent core from the surface and surrounding environment, significantly enhancing quantum yield to as high as 80%, meaning that most of the photons absorbed are re-emitted as fluorescence (Peng, et al., “Epitaxial Growth of Highly Luminescent CdSe/CdS Core/Shell Nanocrystals with Photostability and Electronic Accessibility,” J. Am. Chem. Soc. 119(30):7019-7029 (1997), which is hereby incorporated by reference in its entirety).

In addition to tunable emission wavelengths, other novel optical properties give Q-dots distinct advantages over organic dyes as has already been reported in the literature cited above:
1) Flexible excitation: relative to organic dyes, Q-dots have extremely large excitation spectra (50 to 90-fold higher with excitation at 350 nm), which allows all Q-dot colors to be excited efficiently with a single wavelength or a broadband source (e.g., a lamp). Single wavelength excitation simplifies optical systems for multiplexed Q-dot assays by allowing the use of inexpensive and robust light-sources such as blue solid-state lasers or LEDs.
2) Minimal spectral overlap: emission spectra from organic dyes are broad and asymmetric. In contrast, the symmetric emission spectra of Q-dots greatly reduce cross-talk in multiplexed assays, increasing sensitivity as well as the number of available colors.
3) Controllable surface chemistry: modifiable surface molecules allow all Q-dots to be conjugated using standard chemistries. Since the surface of all Q-dots can be coated with an identical organic layer, differential reactivity is minimized. Surface chemistry can also be tailored to eliminate non-specific binding in bio-assays.
4) High photostability: unlike organic dyes, Q-dots are extremely photostable. Enhanced photostability means that fluorescence can be measured over long periods of time, enhancing detection sensitivity.

The reporter label is connected to its probe using a binding agent-binding partner. Suitable binding agent-binding partner pairs in accordance with the present invention include, without limitation, antibody-antigen binding partners, streptavidin-biotin binding partners, complementary oligonucleotides, amino groups and EDC activated carboxylic acid groups, thiol based binding partners, histidine moieties and nickel-NTA, and other chemical moieties that may be covalently or ionically linked to each other.

The reporter probe sequences need to be designed so that there is no cross hybridization between any of the sequences or their complements, yet the Tm should be high. The preferred embodiment of this invention uses probe sequences of 16 bases. Most 16mer sequences have Tₘ values in the range of 44° C. to 52° C. The preferred embodiment of this invention uses probe sequences of 16 bases which have Tm values between 65° C. and 67° C. The preferred embodiment for selection of these special sequences is illustrated in FIG. 96. One solution to the set of 16 is provided in Table 3 (infra), other permutations and variations of these and closely related sequences may also be used, provided that there is no cross-hybridization between them.

There are different formulations for using the reporter probe sequences. In one formulation, the discriminating LDR probes each have regions with 3 or 4 reporter probe sequences or their complements, and the common probe possesses a binding agent for subsequent capture. Alternatively, the genomic DNA has a binding agent (such as biotin groups) incorporated to allow capture, the discriminating LDR probes each have 2 reporter probe sequences, and the common probes also possess 2 reporter probe sequences. In another formulation, the discriminating LDR probes each have 2 reporter probe sequences, the common probes have standard addressable array-specific portion, and the products are captured on a standard Universal array. Also included in some of these formulations is the option of capturing reporter probes onto the detection sequences using a thermostable ligase to ligate two or more detection probes together.

In addressing the encryption problem, due to the quantitative nature of quantum dot nanocrystals, the use of one, two, or even three of the same color quantum dot nanocrystals can be used to distinguish between ligation products at a given location or address. For the encryption solutions presented below, the number of quantum dot nanocrystals of the same color at a given location or address has been limited to a maximum of two, defined as “doublets” in this application.

The individual primary ligation products or complements thereof may be captured on one or a plurality of solid supports. This takes place after subjecting the primary ligase detection reaction mixture to one or more ligase detection reaction cycles and prior to contacting the primary ligation products and the detection oligonucleotide probes. As a result, each primary ligation product is individually identified.

Paramagnetic beads are suitable solid supports in accordance with the present invention. Where the solid support is a paramagnetic bead, the method further involves recovering the paramagnetic beads by magnetic attraction after capturing, and placing the recovered paramagnetic beads on a microscope slide.

In permitting attachment of ligation products to solid supports, the first oligonucleotide probe of the primary oligonucleotide probe set or sets may have a binding agent which is incorporated in any primary ligation product, and the solid support can have one or more attached binding partner to the binding agent. The ligation product is captured in the solid support by use of conditions effective for the binding agent and its binding partner to become coupled together. As a result, any primary ligation product is immobilized to the solid support.

Suitable binding agent-binding partner pairs in accordance with the present invention include, without limitation, antibody-antigen binding partners, streptavidin-biotin binding partners, complementary oligonucleotides, amino groups and EDC activated carboxylic acid groups, thiol based binding partners, histidine moieties and nickel-NTA, and other chemical moieties that may be covalently or ionically linked to each other.

A complement of the primary ligation product sequence may be captured on the solid support in accordance with the present invention. This involves subjecting the primary ligation product to a polymerase extension reaction after subjecting the primary ligase detection reaction mixture to one or more ligase detection reaction cycles and prior to capturing. Alternatively, the primary ligation product itself can be captured on the solid support.

In this aspect of the present invention, the capture oligonucleotide probes at the particular sites on the solid support may be the same as each other. The capture oligonucleotide probes at the particular sites on the solid support may also each be unique with respect to one another.

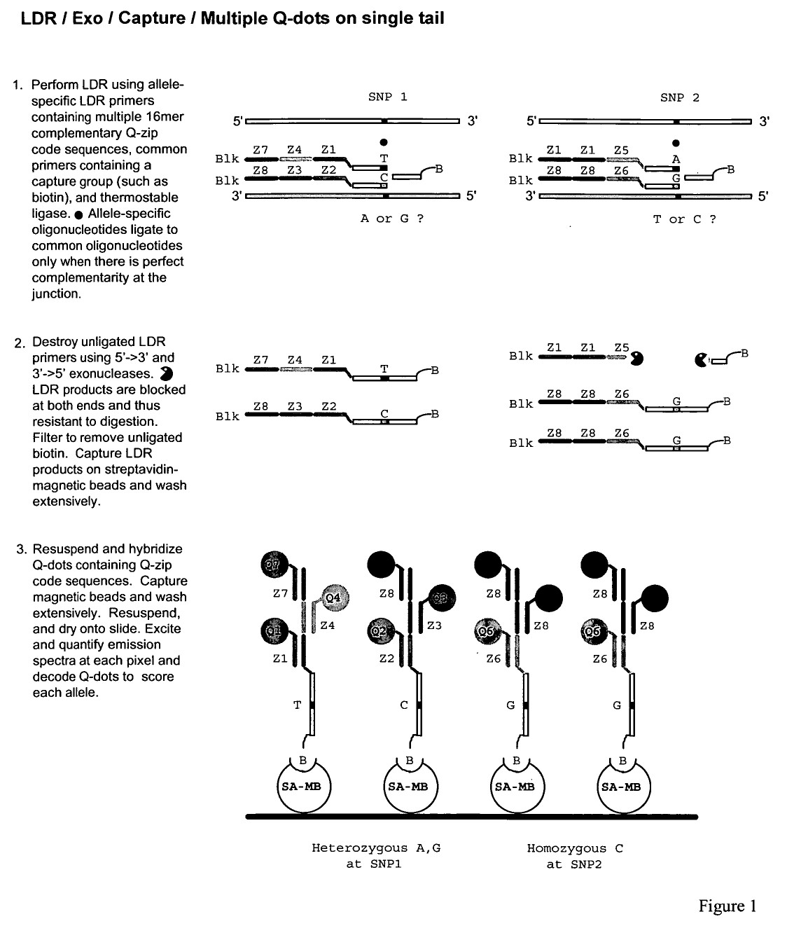

Embodiments of the ligase detection reaction/detection probe hybridization/detection scheme of the present invention are shown in FIGS. 1-6.

FIG. 1 is a flow diagram of an LDR process, in accordance with the present invention, where multiple Q-dots are used on a single tail to distinguish single base differences in nucleic acid molecules. LDR is performed using allele-specific LDR probes containing multiple 16mer complementary Q-Zip portions, common probes containing a capture group (such as biotin), and a thermostable ligase. The allele-specific oligonucleotide probes (Z7,Z4,Z1), (Z8,Z3,Z2), (Z1,Z1,Z5), and (Z8,Z8,Z6) discriminate A, G, T, and C, respectively, on the target nucleic acid molecule. Allele-specific oligonucleotide probes ligate to common oligonucleotide probes only when there is perfect complementarity at the junction. Subsequently, unligated oligonucleotide probes are destroyed using 5′→3′ and 3′→5′ exonucleases. Ligation products are blocked at both ends and are thus resistant to digestion. The remaining biotin-containing fragments are removed using filtration or size-exclusion columns. Ligation products are then captured on streptavidin-magnetic beads and washed extensively. Magnetic beads containing the ligation products are resuspended and hybridized with Q-dot containing Q-Zip code portions. After capture and extensive washing, the magnetic beads are resuspended and dried onto a slide. Emission spectra at each pixel are quantified to decode the presence of specific Q-dots, to score each allele.

The detection results in FIG. 1 show that the sample is heterozygous A, G at SNP1 and homozygous C at SNP2. The A allele at SNP1 is indicated by Q-dots (Q7, Q4, Q1), which are part of detection oligonucleotide probes (Z7, Z4, Z1, respectively). The G allele at SNP1 is indicated by Q-dots (Q8, Q3, Q2), which are part of detection olignucleotide probes (Z8, Z3, Z2, respectively). The C allele at SNP 2 is indicated by the Q-dots (Q8, Q8, Q6,) which are part of detection olignucleotide probes (Z8, Z8, and Z6, respectively).

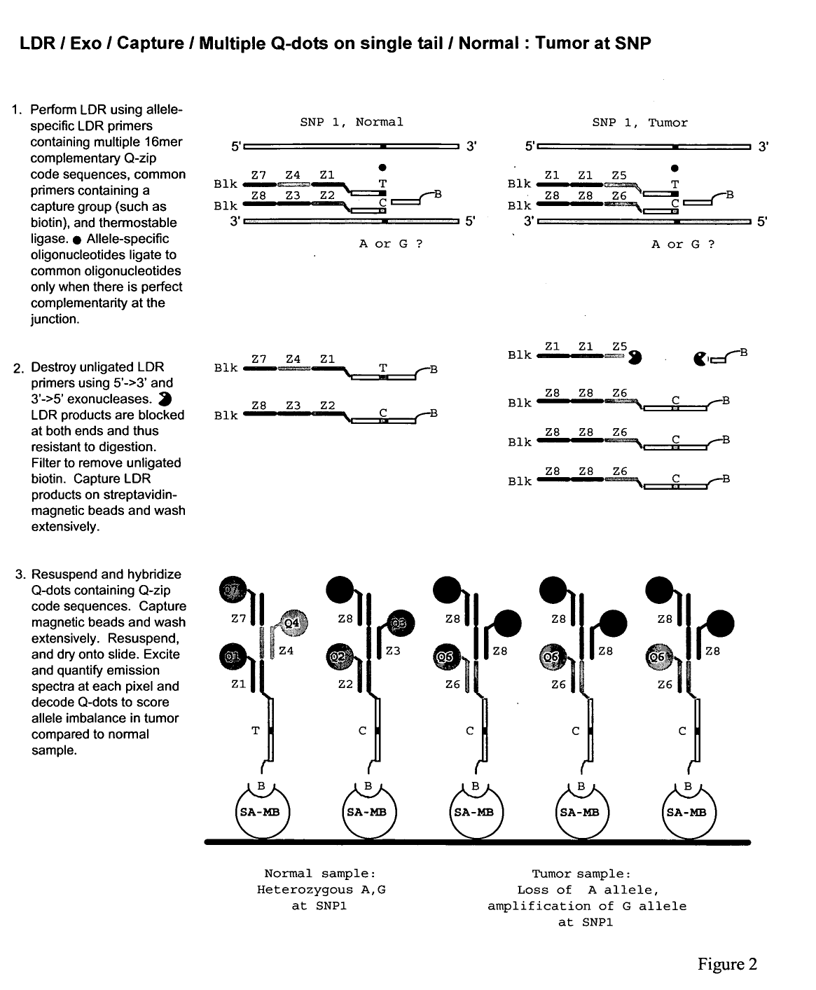

FIG. 2 is a schematic diagram of an LDR process, in accordance with the present invention, where multiple Q-Zip portions containing Q-dots on a single tail are used with microbead capture to score allele imbalance in a tumor sample compared with a normal sample. FIG. 2 is similar to FIG. 1, except the Q-dot encryption is used to score allele imbalance in a tumor sample compared with the normal sample. The detection results in FIG. 2 show that, at SNP1, the normal sample is heterozygous A, G and the tumor sample demonstrates a loss of the A allele and amplification of the G allele. The normal sample has a ratio of Q-dots which are part of detection oligonucleotide probes (i.e. Q7, Q4, Q1)/)(Q8, Q3, Q2) of 1, indicating that the sample is heterozygous for A and G alleles. In the tumor sample, the ratio of Q-dots which are part of detection oligonucleotide probes (i.e. Q1, Q1, Q5)/(Q7, Q4, Q1) is about zero, indicating loss of the A allele. The ratio of Q-dots which are part of detection oligonucleotide probes (i.e. Q8, Q8, Q6)/(Q8, Q3, Q2) is about three, indicating an approximately three-fold amplification of the G allele in the tumor.

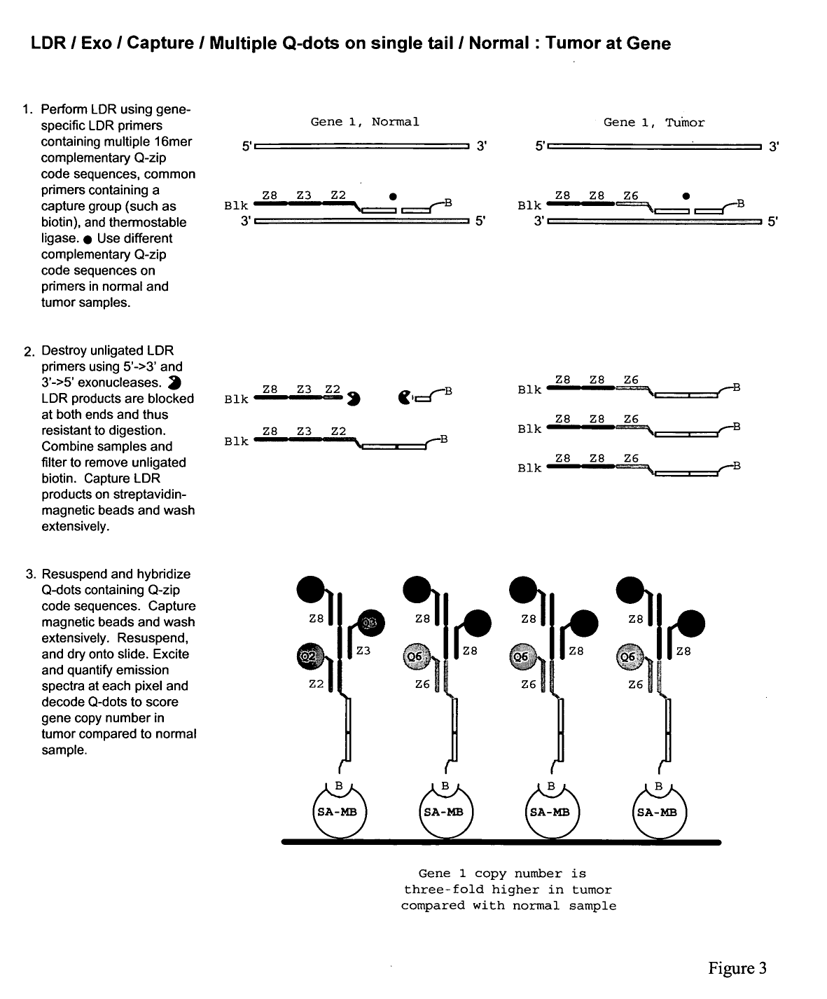

FIG. 3 is a schematic diagram of an LDR process, in accordance with the present invention, where multiple Q-Zip portions containg Q-dots on a single tail are used with microbead capture and a polymerase extension step to score gene copy number in a tumor sample compared with a normal sample. FIG. 3 is similar to FIG. 2, except the Q-dot encryption is used to score gene copy number in a tumor sample compared with the normal sample. The detection results in FIG. 3 show an approximately three-fold amplification of gene 1 in the tumor sample, which is indicated by a ratio of hybridized Q-Zip portions containg Q-dots Q8, Q8, Q6)/(Q8, Q3, Q2) of about three.

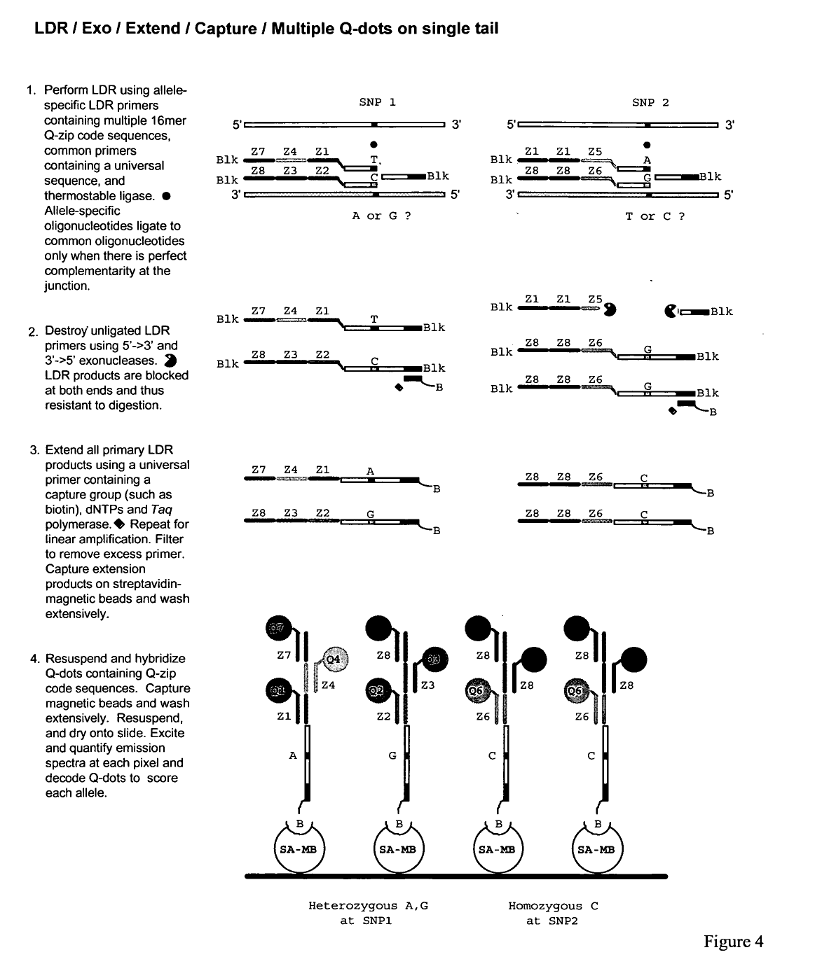

FIG. 4 is a flow diagram of an LDR process, in accordance with the present invention, where multiple Q-Zip portions containing Q-dots are used on a single tail with microbead capture and a polymerase extension step to detect single base differences in nucleic acid molecules. LDR is performed using allele-specific LDR oligonucleotide probes containing multiple 16mer complementary detection probe-specific sequences, common oligonucleotide probes containing a universal sequence, and thermostable ligase. Allele-specific oligonucleotide probes (Z7, Z4, Z1), (Z8, Z3, Z2), (Z1, Z1, Z5), and (Z8, Z8, Z6) discriminate A, G, T, and C, respectively, on the target 3′→5′ nucleotide sequence. Allele-specific oligonucleotide probes ligate to common oligonucleotide probes only when there is perfect complementarity at the junction. Subsequently, unligated LDR oligonucleotide probes are destroyed using 5′→3′ and 3′→5′ exonucleases. Ligation products are blocked at both ends and are thus resistant to digestion. These products may be amplified linearly by extending the primary ligation products using a universal primer containing a capture group (such as biotin), and Taq polymerase. The remaining biotin-containing primers are removed using filtration or size-exclusion columns. Extension products are captured on streptavidin-magnetic beads and washed extensively. Magnetic beads containing the extension products are resuspended and hybridized with Q-dot containing Q-Zip portions. After capture and extensive washing, the magnetic beads are resuspended and dried onto a slide. Emission spectra at each pixel are quantified to decode the presence of specific Q-dots, to score each allele.

The detection results in FIG. 4 show that the sample is heterozygous for A and G at SNP1 and homozygous for C at SNP2. The A allele is indicated by the Q-dots (Q7, Q4, Q1) which are part of detection oligonucleotide probes hybridizing to the ligation product having oligonucleotide probes with Z7, Z4, and Z1, respectively. The G allele is indicated by the Q-dots (Q8, Q3, Q2) which are part of the detection probe hybridizing to the ligation product having oligonucleotide probes Z8, Z3, Z2. The C allele is indicated by Q-dots (Q8, Q8, Q6) which are part of the detection probe hybridizing to the ligation product having oligonucleotide probes with Z8, Z8, Z6.

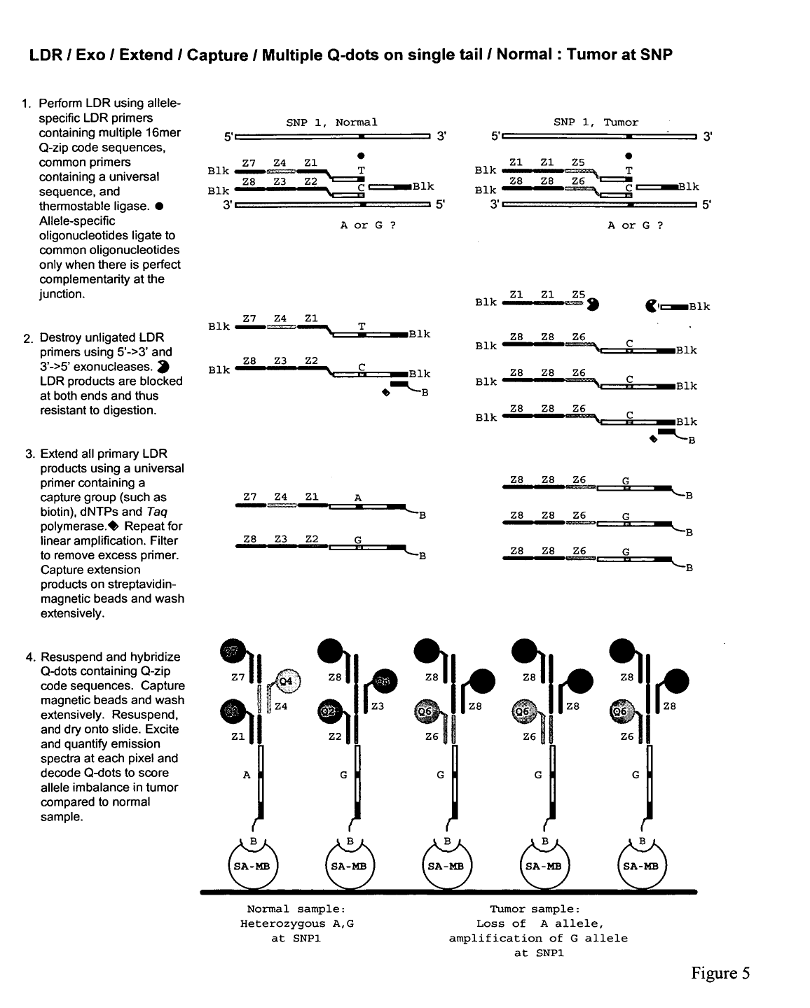

FIG. 5 is a schematic diagram of an LDR process, in accordance with the present invention, where double Q-Zip portion containing Q-dots on a single tail are used with microbead capture and a polymerase extension step to score allele imbalance in a tumor sample compared with a normal sample. FIG. 5 is similar to FIG. 4, except the Q-dot encryption is used to score allele imbalance in a tumor sample compared with the normal sample. The detection results in FIG. 5 show that at SNP1 the normal sample is heterozygous A, G and the tumor sample demonstrates a loss of the A allele and amplification of the G allele. The normal sample has a ratio of Q-dots which are part of detection oligonucleotide probes (i.e. (Q7, Q4, Q1)/(Q8, Q3, Q2)) of 1, indicating that the sample is heterozygous for the A and G alleles. In FIG. 5 the ratio of Q-dots which are part of the detection probe hybridizing to a ligation product (i.e. Q1, Q1, Q5)/(Q7, Q4, Q1)) is about zero, indicating loss of the A allele in the tumor. The ratio of Q-dots which are part of the detection probe hybridizing to a ligation product (i.e. (Q8, Q8, Q6)/(Q8, Q3, Q2)) is about three, indicating an approximately three-fold amplification of the G allele in the tumor.

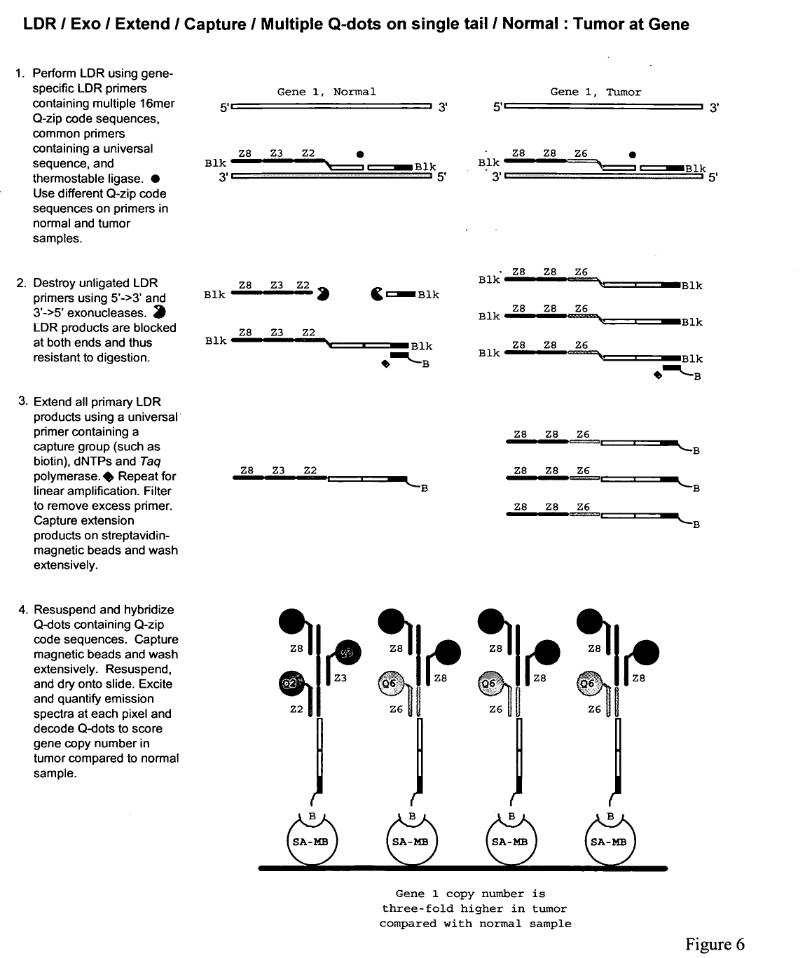

FIG. 6 is a schematic diagram of an LDR process, in accordance with the present invention, where multiple Q-Zip portions containing Q-dots on a single tail are used with microbead capture and a polymerase extension step to score gene copy number in a tumor sample compared with a normal sample. FIG. 6 is similar to FIG. 4, except the Q-dot encryption is used to score gene copy number in a tumor sample compared with the normal sample. In the initial LDR reaction a given gene is interrogated with an allele-specific encrypted oligonucleotide probes in the normal sample in a first tube. The detection results in FIG. 6 show an approximately three-fold amplification of gene 1 in the tumor sample (indicated by a ratio of Q-dots (i.e. (Q8, Q8, Q6)/(Q8, Q3, Q2)) which are part of detection oligonucleotide probes, of about three).

Solid supports containing capture oligonucleotide probes complementary to an addressable array-specific portion of the ligation product (and, initially, in the primary oligonucleotide probe sets) can also used in carrying out the present invention. In this aspect of the present invention, the ligation product is contacted with the solid support at a temperature of 45-90° C. and for a time period of up to 60 minutes. Hybridization may be accelerated by adding volume exclusion or chaotropic agents. When an array consists of dozens to hundreds of addresses, it is important that the correct ligation products have an opportunity to hybridize to the appropriate address. This may be achieved by the thermal motion of oligonucleotides at the high temperatures used, by mechanical movement of the fluid in contact with the array surface, or by moving the oligonucleotides across the array by electric fields. After hybridization, the array is washed sequentially with a low stringency wash buffer and then a high stringency wash buffer.

It is important to select capture oligonucleotide probes and addressable array-specific portions which will hybridize in a stable fashion. This requires that the oligonucleotide probe sets and the capture oligonucleotides be configured so that the oligonucleotide sets hybridize to the target nucleic acid molecules at a temperature less than that which the capture oligonucleotides hybridize to the addressable array-specific portions. Unless the oligonucleotides are designed in this fashion, false positive signals may result due to capture of adjacent unreacted oligonucleotides from the same oligonucleotide set which are hybridized to the target.

The solid support can be made from a wide variety of materials. The substrate may be biological, nonbiological, organic, inorganic, or a combination of any of these, existing as particles, strands, precipitates, gels, sheets, tubing, spheres, containers, capillaries, pads, slices, films, plates, slides, discs, membranes, etc. The substrate may have any convenient shape, such as a disc, square, circle, etc. The substrate is preferably flat but may take on a variety of alternative surface configurations. For example, the substrate may contain raised or depressed regions on which the synthesis takes place. The substrate and its surface preferably form a rigid support on which to carry out the reactions described herein. The substrate and its surface is also chosen to provide appropriate light-absorbing characteristics. For instance, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, polycarbonate, polyethylene, polypropylene, polyvinyl chloride, poly(methyl acrylate), poly(methyl methacrylate), or combinations thereof. Other substrate materials will be readily apparent to those of ordinary skill in the art upon review of this disclosure. In a preferred embodiment, the substrate is flat glass or single-crystal silicon.

A variety of commercially-available materials, which include suitably modified glass, plastic, or carbohydrate surfaces or a variety of membranes, can be used. Depending on the material, surface functional groups (e.g., silanol, hydroxyl, carboxyl, amino) may be present from the outset (perhaps as part of the coating polymer), or will require a separate procedure (e.g., plasma amination, chromic acid oxidation, treatment with a functionalized side chain alkyltrichlorosilane) for introduction of the functional group.

The surface of the functionalized substrate is preferably provided with a layer of linker molecules, although it will be understood that the linker molecules are not required elements of the invention. The linker molecules are preferably of sufficient length to permit polymers in a completed substrate to interact freely with molecules exposed to the substrate. The linker molecules should be 6-50 atoms long to provide sufficient exposure. The linker molecules may be, for example, aryl acetylene, ethylene glycol oligomers containing 2-10 monomer units, diamines, diacids, amino acids, or combinations thereof.

Further details regarding solid supports, functional groups, and linker are set forth in U.S. patent application Ser. No. 08/794,851 to Barany et. al., and WO 97/31256 to Barany et. al., which are hereby incorporated by reference in their entirety. Techniques for improving the performance of addressable arrays is set forth in U.S. Pat. No. 6,506,594 to Barany et. al., which is hereby incorporated by reference in its entirety.

In all aspects of the present invention, a plurality of primary oligonucleotide probe sets may be utilized, with each set characterized by (a) the first oligonucleotide probe being identical in each oligonucleotide probe set and (b) the second oligonucleotide probes in each set having a target-specific portion which is different in each second oligonucleotide probe at a location where single-base changes, insertions, deletions, or translocations occur.

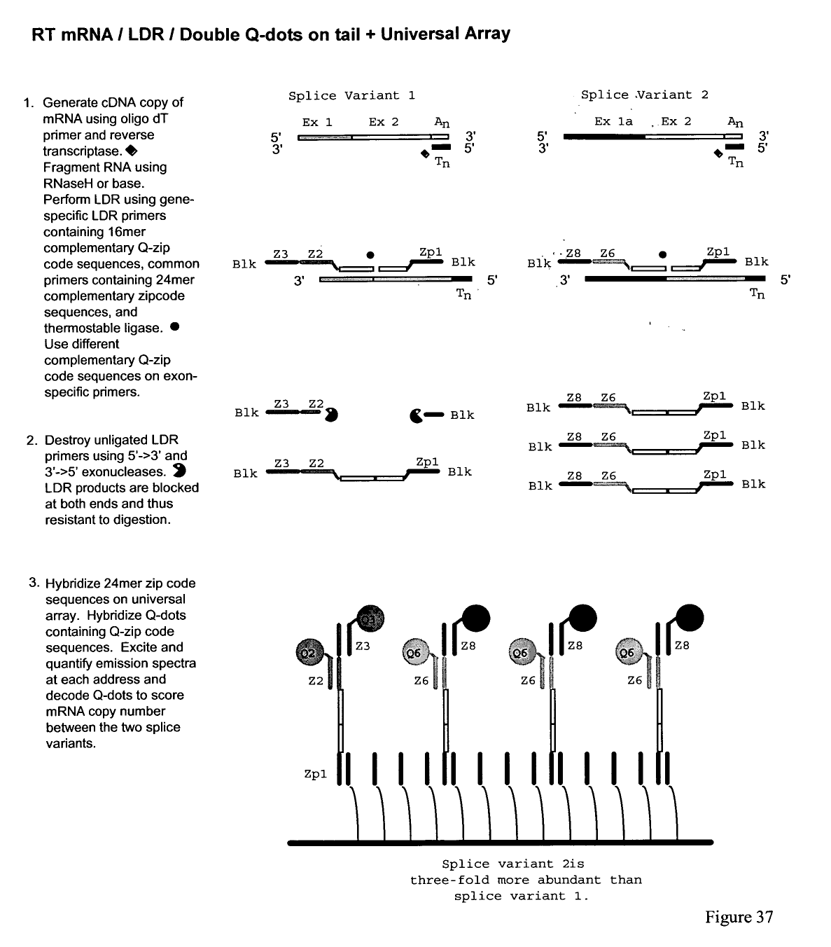

Examples of carrying out the ligase detection reaction/detection oligonucleotide probe hybridization/detection scheme of the present invention with an addressable array are shown in FIGS. 37-53.

FIG. 37 is a schematic diagram of an LDR process, in accordance with the present invention, where double Q-Zip portions containing Q-dots on a single tail are used with a universal array to distinguish and quantify splice variants on mRNA. A cDNA copy of the mRNA is generated using reverse transcriptase on an oligo dT primer containing a capture group (e.g., biotin) on the 5′ end. RNA is fragmented using RNaseH or base. Excess linkers are removed using filtration or size-exclusion columns. LDR is performed directly on the resulting cDNA using exon-specific oligonucleotide probes containing 16mer complementary Q-Zip portions, common oligonucleotide probes containing addressable array-specific portions, and thermostable ligase. Different Q-Zip portions are used on exon-specific oligonucleotide probes. Unligated oligonucleotide probes are destroyed with exonucleases. Ligation products are captured on a universal array by hybridizing the 24-mer addressable array-specific portions to capture probes on the array. Q-Zip portions containing Q-dots are hybridized to ligation products on the universal array. Emission spectra at each location or address on the array are quantified to decode the presence of specific Q-dots, to score each splice variant.

The detection results in FIG. 37 show an approximately three-fold higher expression of the Ex1a splice variant in the sample (indicated by a ratio of Q-Zip code signals (Q8,Q6)/(Q3,Q2) at address Zp1 of about three).

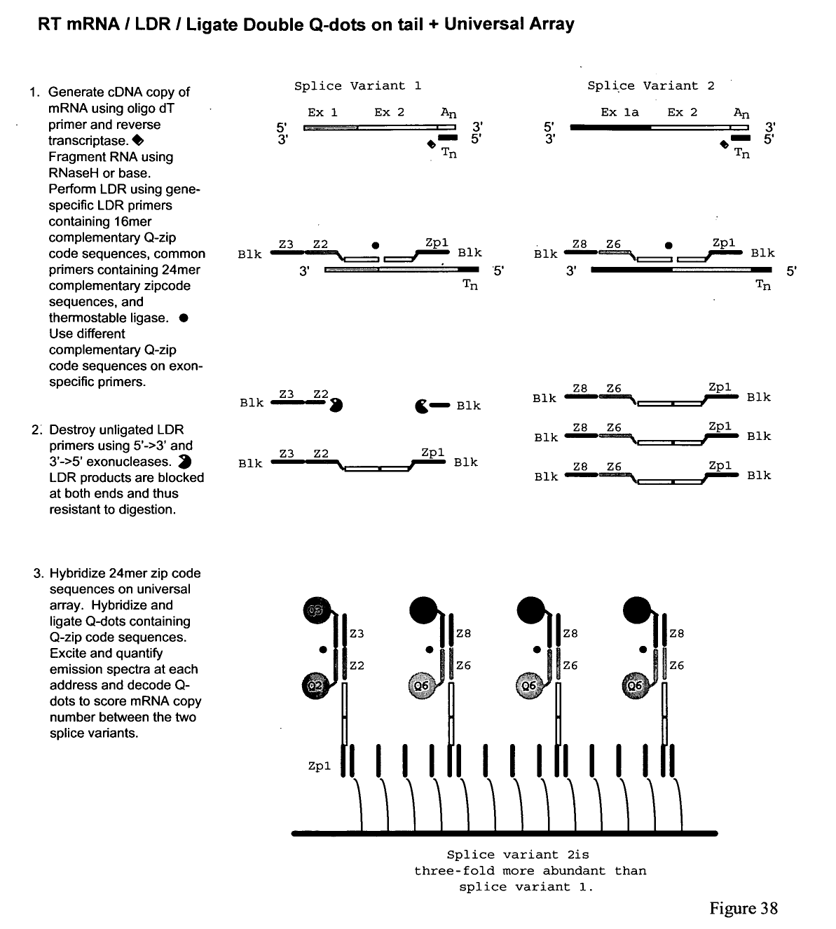

FIG. 38 is a schematic diagram of an LDR process, in accordance with the present invention, where ligated double Q-Zip portions containing Q-dots on a single tail are used with a universal array to distinguish and quantify splice variants on mRNA. FIG. 38 is similar to FIG. 37, but with the addition of ligating adjacent Q-dot containing Q-Zip portions on adjacent 16mer complementary Q-Zip portions in the last step. The detection results in FIG. 38 show an approximately three-fold amplification of gene 1 in the tumor sample (indicated by a ratio of Q-dots (Q8,Q6)/(Q3,Q2) on ligated Q-Zip portions at address Zp1 of about three).

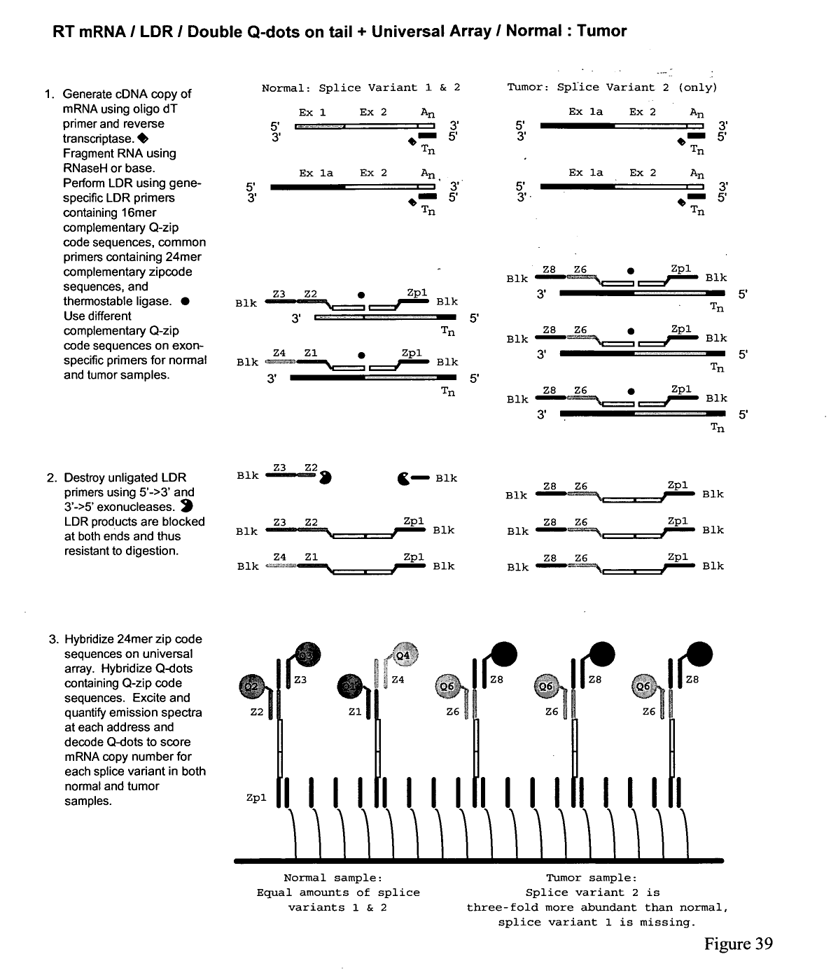

FIG. 39 is a schematic diagram of an LDR process, in accordance with the present invention, where double Q-Zip portions containing Q-dots on a single tail are used with a universal array to distinguish and quantify splice variants on mRNA in a tumor sample compared with a normal sample. FIG. 39 is similar to FIG. 37, except that the Q-dot encryption is used to score splice variants in a tumor sample compared with a normal sample. cDNAs are prepared as described above. Tumor and normal samples are kept separate. In the initial LDR reaction, a given splice junction is interrogated with two allele-specific encrypted oligonucleotide probes in the normal sample in a first tube. The same splice junction is interrogated with two different allele-specific encrypted oligonucleotide probes in the tumor sample in a second tube. Although multiple different splice variants are detected in each tube, for simplicity only splice variants 1 and 2 are shown. After the ligation step, the ligation products are combined in a single tube and treated with exonucleases to destroy unligated oligonucleotide probes. Ligation products are hybridized onto a universal array via the 24-mer addressable array-specific portion on the ligation product and capture oligonucleotide probe on the solid support. Q-dot containing Q-Zip portions are hybridized to the ligation products on the universal array. Emission spectra at each address are quantified to decode the presence of specific Q-dots, to score splice variants in the tumor compared to the normal sample.

The detection results in FIG. 39 show that the tumor demonstrates a loss of the Ex1 variant and an approximately three-fold higher expression of the Ex1a splice variant. In FIG. 39, the ratio of hybridized Q-Zip probes (Q1,Q5)/(Q4,Q1) at the Zp1 address is about zero, indicating a loss of the Ex1 variant in the tumor. The ratio of Q-dots on hybridized Q-Zip portions (i.e. (Q8,Q6)/(Q3,Q2)) at the Zp1 address is about three, indicating an approximately three-fold higher expression of the Ex1a splicing variant in the tumor.

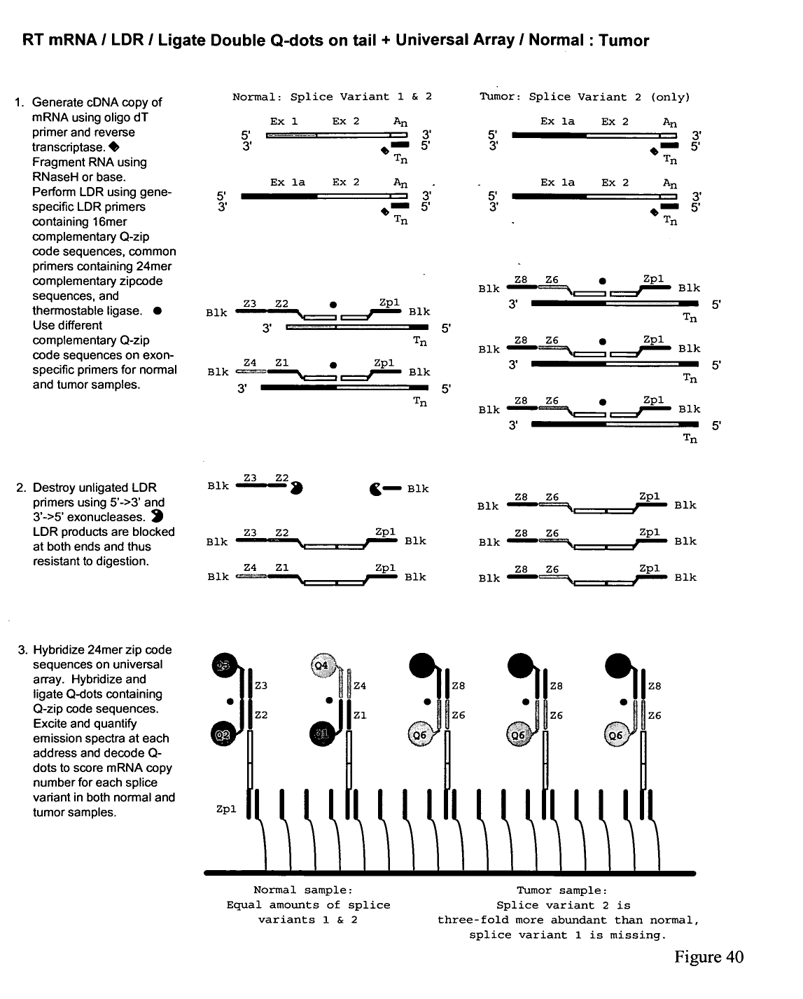

FIG. 40 is a schematic diagram of an LDR process, in accordance with the present invention, where ligated double Q-Zip portions containing Q-dots on a single tail are used with a universal array to score splice variants on mRNA in a tumor sample compared with a normal sample. FIG. 40 is similar to FIG. 39, but with the addition of ligating adjacent Q-dot containing Q-Zip portions on adjacent 16mer complementary Q-Zip portions in the last step.

The detection results in FIG. 40 show that the tumor demonstrates a loss of the Ex1 variant and an approximately three-fold higher expression of the Ex1 a splicing variant. In FIG. 40 the ratio of hybridized ligated Q-Zip portions containing Q-dots (Q1,Q5)/(Q4,Q1) at the Zp1 address is about zero, indicating loss of the Ex1 variant in the tumor. The ratio of hybridized ligated Q-Zip portions containing Q-dots (Q8,Q6)/(Q3,Q2) at the Zp1 address is about three, indicating an approximately three-fold higher expression of the Ex1a splicing variant in the tumor.

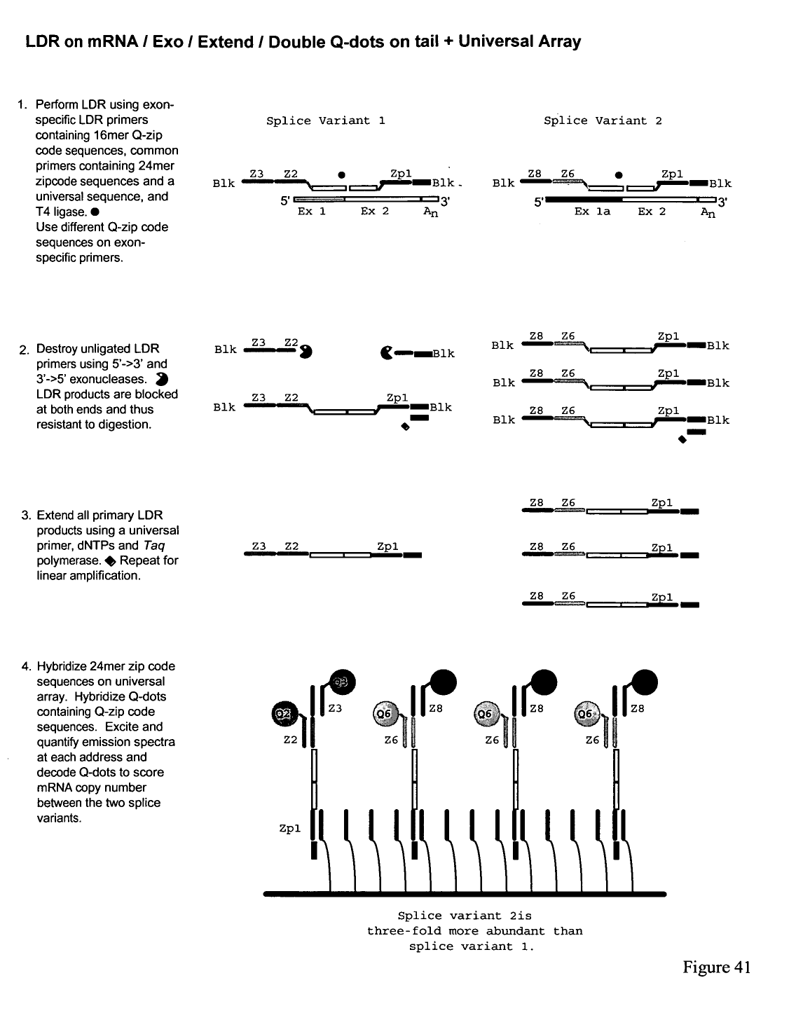

FIG. 41 is a flow diagram of an LDR process, in accordance with the present invention, where double Q-Zip portions containing Q-dots on a single tail are used with a universal array to distinguish and quantify splice variants directly on mRNA. Ligation is performed directly on mRNA using exon-specific oligonucleotide probes containing 16mer Q-Zip portions, common oligonucleotide probes containing addressable array-specific portions and a universal primer site, and T4 ligase. Different Q-Zip portions containing Q-dots are used on exon-specific oligonucleotide probes. Unligated probes are destroyed with exonucleases. Subsequently, all primary ligation products are extended using a universal primer, dNTPs, and Taq polymerase. The extension step is repeated for linear amplification of ligation products as necessary. Extension products are hybridized onto a universal array via the 24-mer zip code sequences. Extension products on the universal array are hybridized with Q-dot containing Q-Zip portions. Emission spectra at each address are quantified to decode the presence of specific Q-dots, to score each splice variant. The detection results in FIG. 41 show an approximately three-fold higher expression of the Ex1a splicing variant in the sample (indicated by a ratio of Q-dots on Q-Zip portions (Q8,Q6)/(Q3,Q2) at address Zp1 of about three).

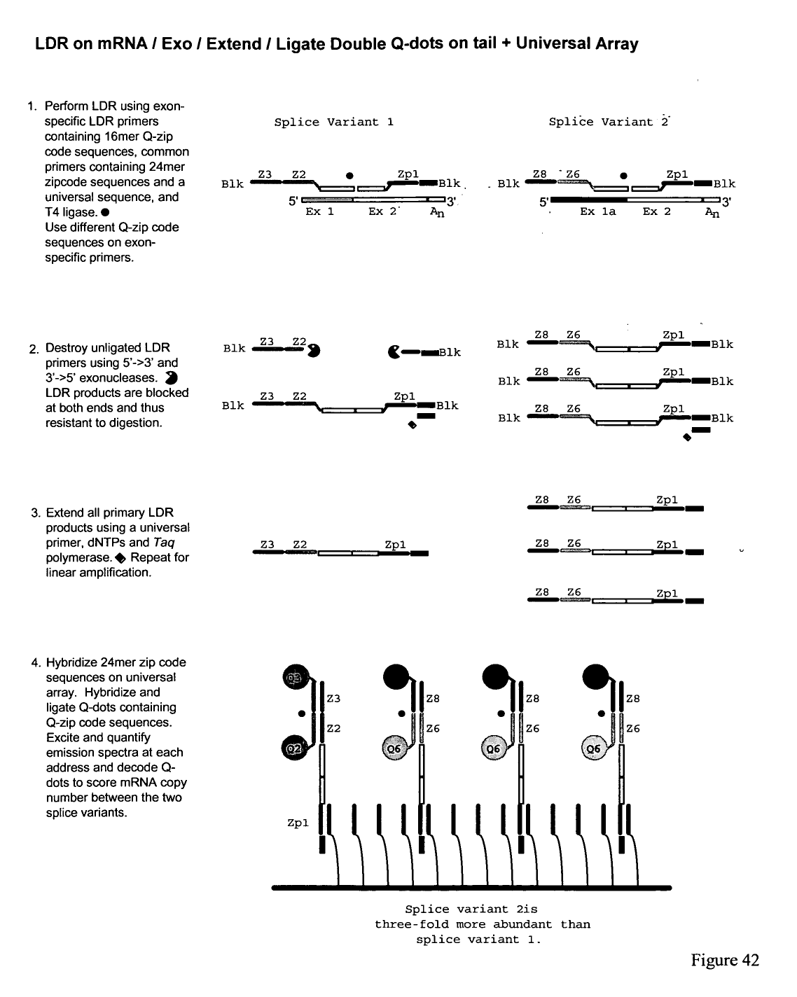

FIG. 42 is a schematic diagram of an LDR process, in accordance with the present invention, where ligated double Q-Zip portions containing Q-dots on a single tail are used with a universal array to distinguish and quantify splice variants directly on mRNA. FIG. 42 is similar to FIG. 41, but with the addition of ligating adjacent Q-dot containing Q-Zip portions containing Q-dots in the last step. The detection results in FIG. 42 show an approximately three-fold higher expression of the Ex1a splicing variant in the tumor sample (indicated by a ratio of ligated Q-Zip portions containing Q-dots (i.e. (Q8,Q6)/(Q3,Q2)) at address Zp1 of about three).

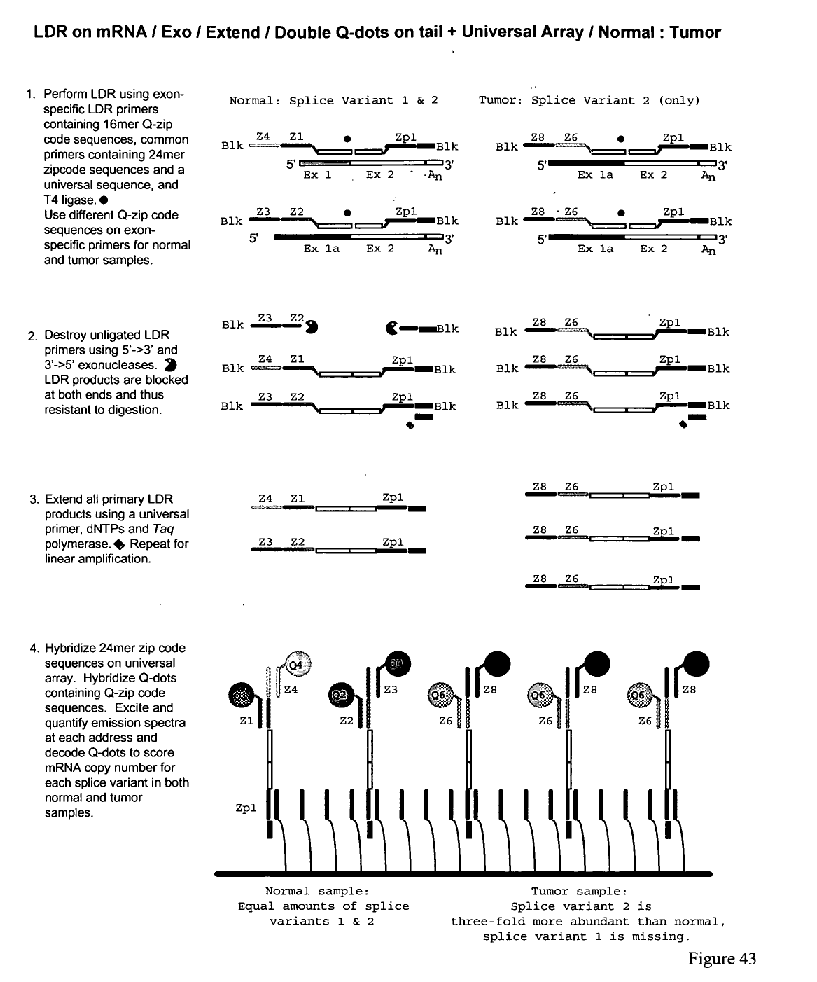

FIG. 43 is a schematic diagram of an LDR process, in accordance with the present invention, where double Q-Zip portions containing Q-dots on a single tail are used with a universal array to score splice variants directly on mRNA in a tumor sample compared with a normal sample. FIG. 43 is similar to FIG. 41, except the Q-dot encryption is used to score splice variants in the tumor sample compared with the normal sample. Tumor and normal samples are kept separate. In the initial ligation reaction a given splice junction is interrogated with two allele-specific encrypted oligonucleotide probes in the normal sample in a first tube. The same splice junction is interrogated with two different allele-specific encrypted oligonucleotide probes in the tumor sample in a second tube. Although multiple different splice variants are detected in each tube, for simplicity only splice variants 1 and 2 are shown. After the ligation step, the ligation products are combined in a single tube and treated with exonucleases to destroy unligated probes. Subsequently, all primary ligation products are extended using a universal primer, dNTPs, and Taq polymerase. The extension step is repeated for linear amplification of ligation products as necessary. Extension products are hybridized onto a universal array via the 24-mer zip code sequences. Extension products on the universal array are hybridized with Q-dots containing Q-Zip portions containing Q-dots. Emission spectra at each address are quantified to decode the presence of specific Q-dots, to score splice variants in the tumor compared to the normal sample.

The detection results in FIG. 43 show that the tumor demonstrates a loss of the Ex1 variant and an approximately three-fold higher expression of the Ex1a splicing variant. In FIG. 43, the ratio of Q-dots attached to Q-Zip portions (Q1,Q5)/(Q4,Q1) hybridized at the Zp1 address is about zero, indicating loss of the Ex1 variant in the tumor. The ratio of Q-dots contained on Q-Zip portions (Q8,Q6)/(Q3,Q2) hybridized at the Zp1 address is about three, indicating an approximately three-fold higher expression of the Ex1a splicing variant in the tumor.

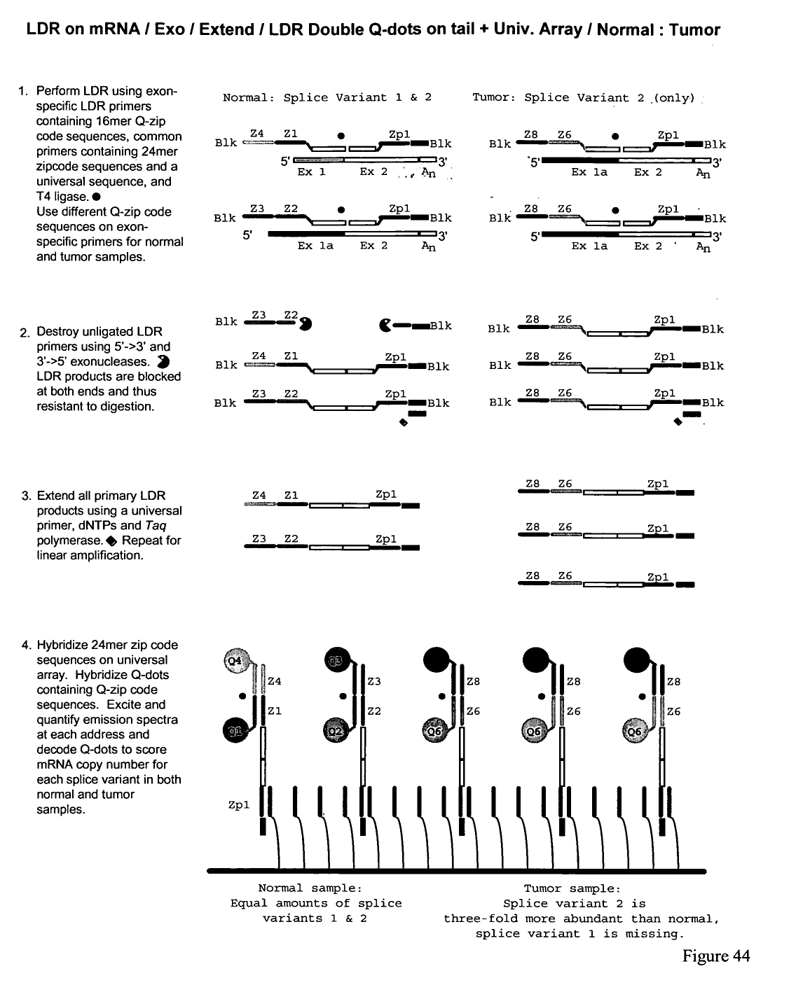

FIG. 44 is a schematic diagram of an LDR process, in accordance with the present invention, where ligated double Q-Zip portions containing Q-dots on a single tail are used with a universal array to score splice variants directly on mRNA in a tumor sample compared with a normal sample. FIG. 44 is similar to FIG. 43, but with the addition of ligating adjacent Q-dot containing Q-Zip portions on adjacent 16mer complementary Q-Zip portions in the last step.

The detection results in FIG. 44 show that the tumor demonstrates a loss of the Ex1 variant and an approximately three-fold higher expression of the Ex1 a splicing variant. In FIG. 44 the ratio of ligated Q-Zip portions containing Q-dots (Q1,Q5)/(Q4,Q1) hybridized at the Zp1 address is about zero, indicating loss of the Ex1 variant in the tumor. The ratio of ligated Q-Zip portions containing Q-dots (Q8,Q6)/(Q3,Q2) hybridized at the Zp1 address is about three, indicating an approximately three-fold higher expression of the Ex1a splicing variant in the tumor.

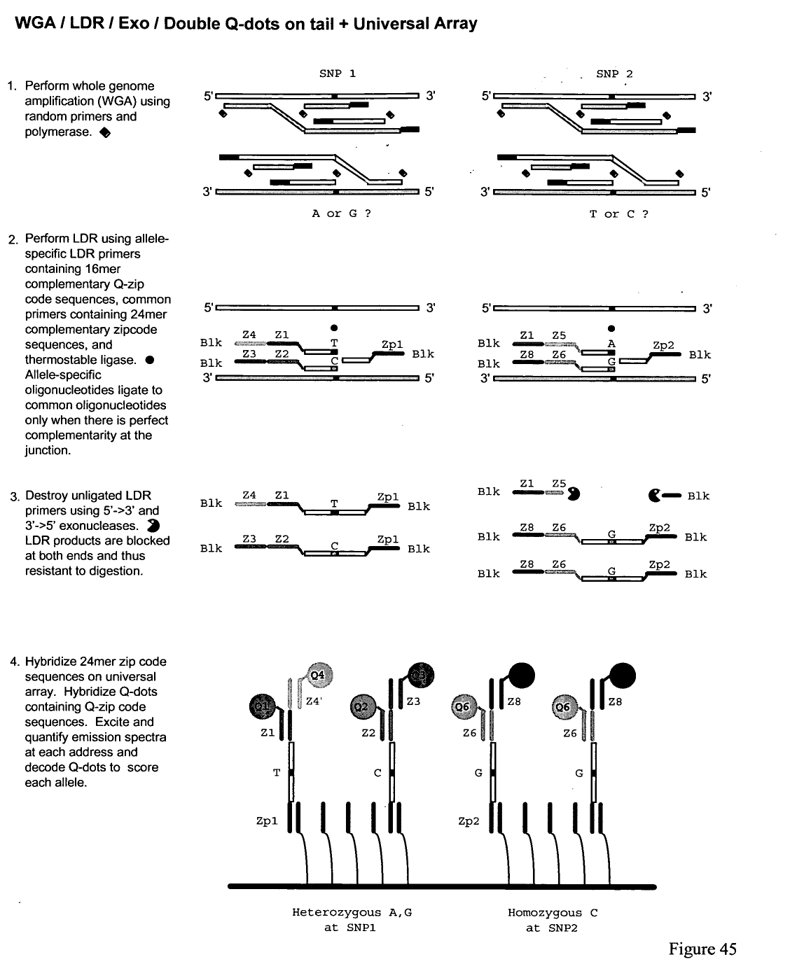

FIG. 45 is a flow diagram of an LDR process, in accordance with the present invention, where whole genome amplification and double Q-Zip portions containing Q-dots on a single tail are used with universal array capture to detect single base differences directly from genomic DNA. Whole genome amplification is performed using random primers and polymerase. LDR is performed using allele-specific oligonucleotide probes containing complementary 16mer Q-Zip portions, common oligonucleotide probes containing complementary 24mer addressable array-specific portions, and thermostable ligase. Allele-specific oligonucleotide probes (i.e. (Z4,Z1), (Z3,Z2), (Z1,Z5), and (Z8,Z6)) discriminate A, G, T, and C, respectively, on the target 3′→5′ nucleotide sequence. The allele-specific oligonucleotide probes ligate to common oligonucleotide probes only when there is perfect complementarity at the junction. Subsequently, unligated oligonucleotide probes are destroyed using 5′→3′ and 3′→5′ exonucleases. Ligation products are blocked at both ends and thus are resistant to digestion. Ligation products are captured on a universal array via the 24-mer addressable array-specific portions in the ligation product and capture oligonucleotide probes on the solid support. Ligation products on the universal array are hybridized with Q-dot containing Q-Zip portions. Emission spectra at each address are quantified to decode the presence of specific Q-dots to score each allele.

The detection results in FIG. 45 show that the sample is heterozygous A, G at SNP1 and homozygous C at SNP2. The A allele is indicated by the presence of Q-Zip portions containing Q-dots (Q4,Q1) at address Zp1. The G allele is indicated by the presence of Q-Zip portions containing Q-dots (Q3,Q2) at address Zp1. The C allele is indicated by the presence of Q-Zip portions containing Q-dots (Q8,Q6) at address Zp2.

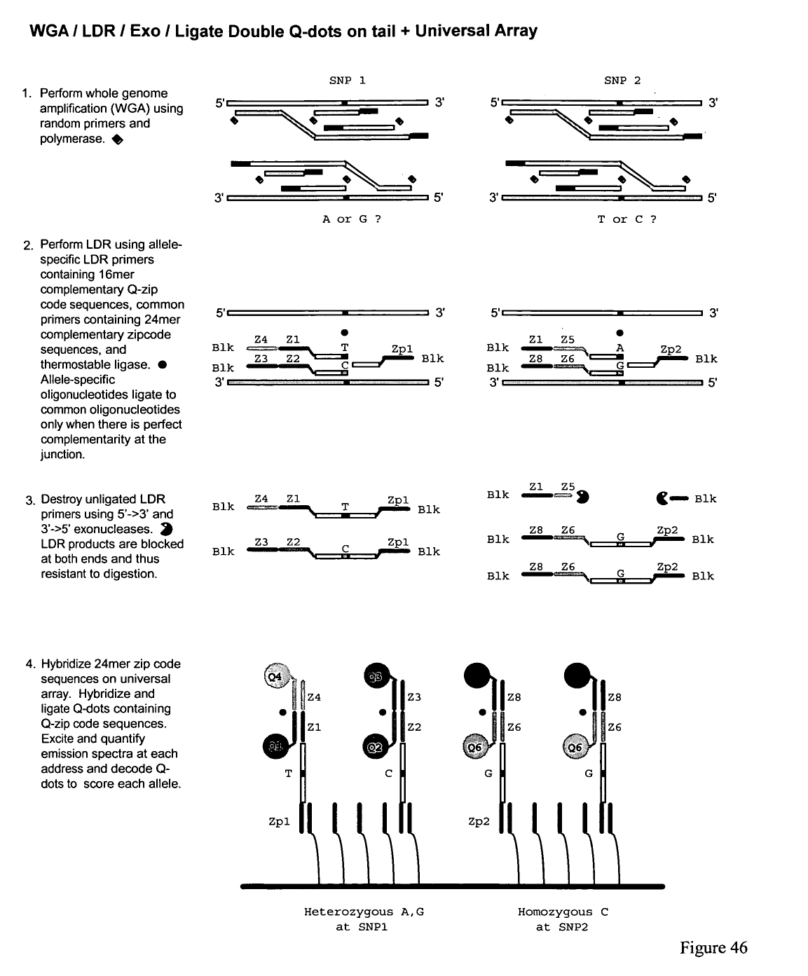

FIG. 46 is a schematic diagram of an LDR process, in accordance with the present invention, where whole genome amplification and ligated double Q-Zip portions containing Q-dots on a single tail are used with universal array capture to distinguish single base differences in nucleic acid molecules. FIG. 46 is similar to FIG. 45, but with the addition of ligating adjacent Q-dot containing Q-Zip portions in the last step.

The detection results in FIG. 46 show that the sample is heterozygous A, G at SNP1 and homozygous C at SNP2. The A allele is indicated by the presence of ligated Q-Zip portions containing Q-dots (Q4,Q1) at address Zp1. The G allele is indicated by the presence of ligated Q-Zip portions containing Q-dots (Q3,Q2) at address Zp1. The C allele is indicated by the presence of Q-Zip portions containing Q-dots (Q8,Q6) at address Zp2.

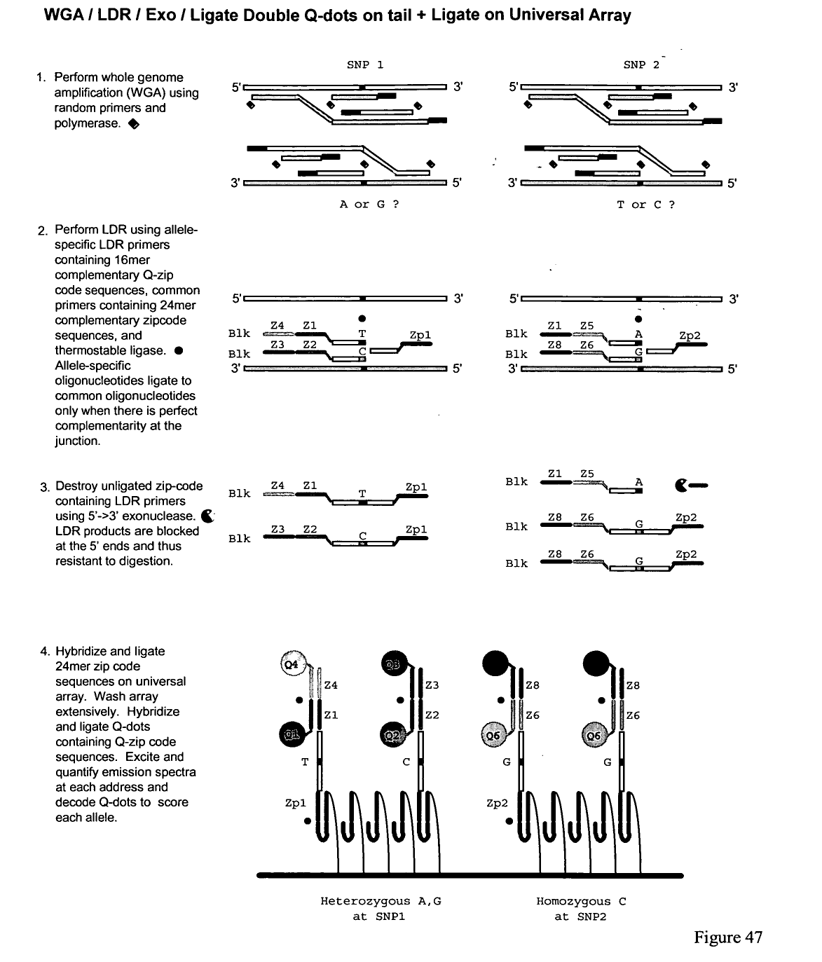

FIG. 47 is a schematic diagram of an/LDR process, in accordance with the present invention, where whole genome amplification and ligated double Q-Zip portions containing Q-dots on a single tail are used with ligation to universal array capture to distinguish single base differences in nucleic acid molecules. FIG. 47 is similar to FIG. 46, but with the addition of ligating the captured complementary addressable array-specific portions to their respective capture probes on the array. In order to allow ligation, the 3′ end of the addressable array-specific portion needs to remain unblocked and, consequently only a 5′→3′ exonuclease is used to destroy unligated versions of this oligonucleotide probe. Alternatively, the 3′ end of the addressable array-specific portion may be synthesized with a thiophosphate group or other modification that inhibits 3′→5′ exonuclease activity, but not ligation. After ligation of ligation products to the array, unligated oligonucleotide probes containing complementary Q-Zip portions are removed by a stringent wash step. Subsequently, Q-dots containing adjacent Q-Zip portions are ligated on adjacent 16mer complementary Q-Zip portions in the last step.

The detection results in FIG. 47 show that the sample is heterozygous A, G at SNP1 and homozygous C at SNP2. The A allele is indicated by the presence of ligated Q-Zip portions containing Q-dots (Q4,Q1) at address Zp1. The G allele is indicated by the presence of ligated Q-Zip portions containing Q-dots (Q3,Q2) at address Zp1. The C allele is indicated by the presence of Q-Zip portions containing Q-dots (Q8,Q6) at address Zp2.

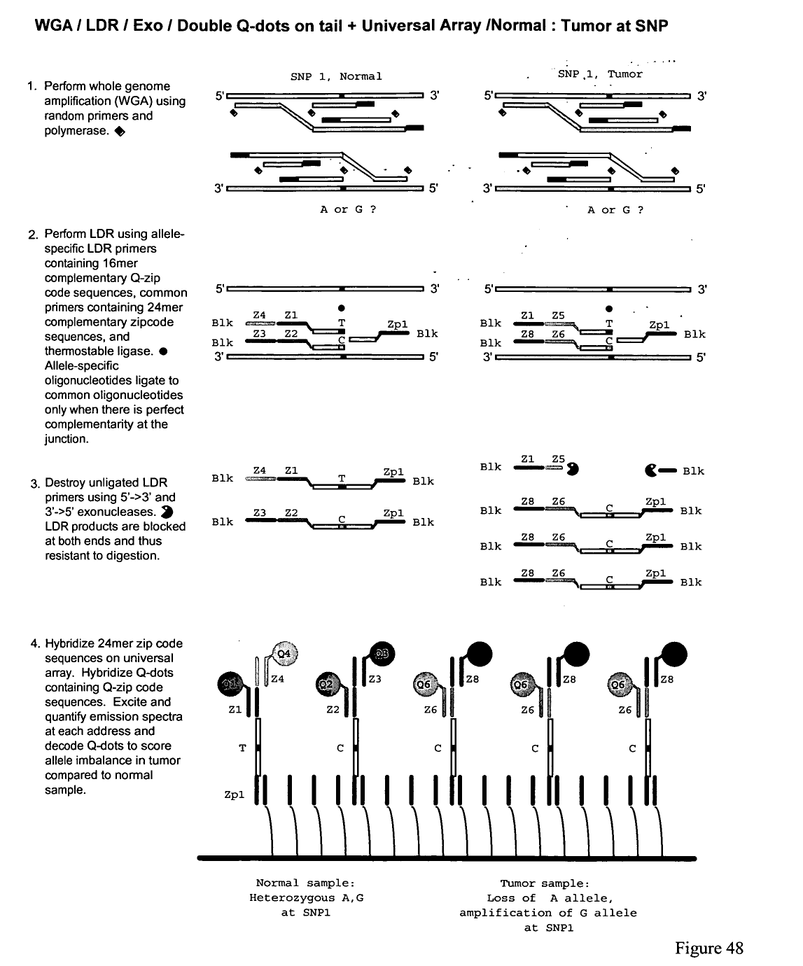

FIG. 48 is a schematic diagram of an LDR process, in accordance with the present invention, where whole genome amplification and double Q-Zip portions containing Q-dots on a single tail are used with universal array capture to score allele imbalance in a tumor sample compared with a normal sample. This figure is similar to FIG. 45, except the Q-dot encryption is used to score allele imbalance in a tumor sample compared with the normal sample. In the initial LDR reaction a given single base difference is interrogated with two allele-specific encrypted oligonucleotide probes in the normal sample in a first tube. The same single base difference is interrogated with two different allele-specific encrypted oligonucleotide probes in the tumor sample in a second tube. Although multiple different single base differences are detected in each tube, for simplicity, only SNP1 is shown. The ligation products are combined in a single tube and the exonuclease digestion and remaining procedure is the same as described in FIG. 45. Emission spectra at each address are quantified to decode the presence of specific Q-dots to score allele imbalance in the tumor compared to the normal sample.

The detection results in FIG. 48 show that at SNP1 the normal sample is heterozygous A, G and the tumor sample demonstrates a loss of the A allele and amplification of the G allele. In FIG. 48, the ratio of Q-Zip portions containing Q-dots (Q1,Q5)/(Q4,Q1) at address Zp1 is about zero, indicating loss of the A allele in the tumor. The ratio of Q-Zip portions containing Q-dots (Q8,Q6)/(Q3,Q2) at address Zp1 is about three, indicating an approximately three-fold amplification of the G allele in the tumor.

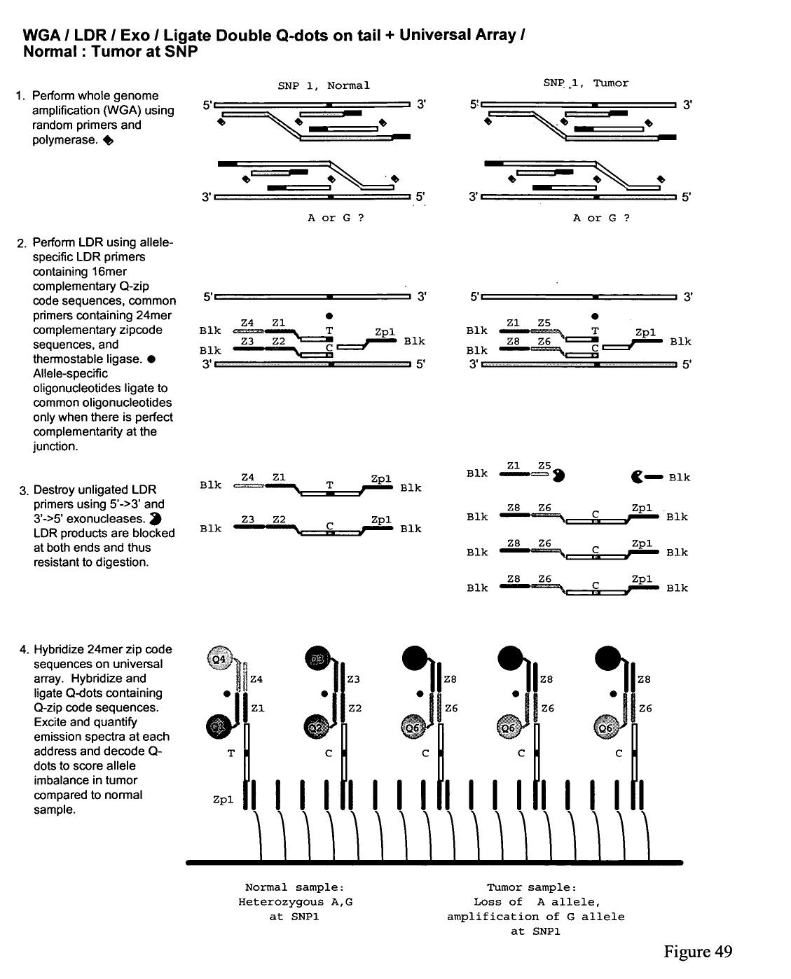

FIG. 49 is a schematic diagram of an LDR process, in accordance with the present invention, where whole genome amplification and ligated double Q-Zip portions containing Q-dots on a single tail are used with universal array capture to score allele imbalance in a tumor sample compared with a normal sample. FIG. 49 is similar to FIG. 48, but with the addition of ligating adjacent Q-dot containing Q-Zip portions in the last step.

The detection results in FIG. 49 show that at SNP1 the normal sample is heterozygous A, G and the tumor sample demonstrates a loss of the A allele and amplification of the G allele. In FIG. 49, the ratio of ligated Q-Zip portions containing Q-dots (Q1,Q5)/(Q4,Q1) at address Zp1 is about zero, indicating loss of the A allele in the tumor. The ratio of ligated Q-Zip portions containing Q-dots (Q8,Q6)/(Q3,Q2) at address Zp1 is about three, indicating an approximately three-fold amplification of the G allele in the tumor.

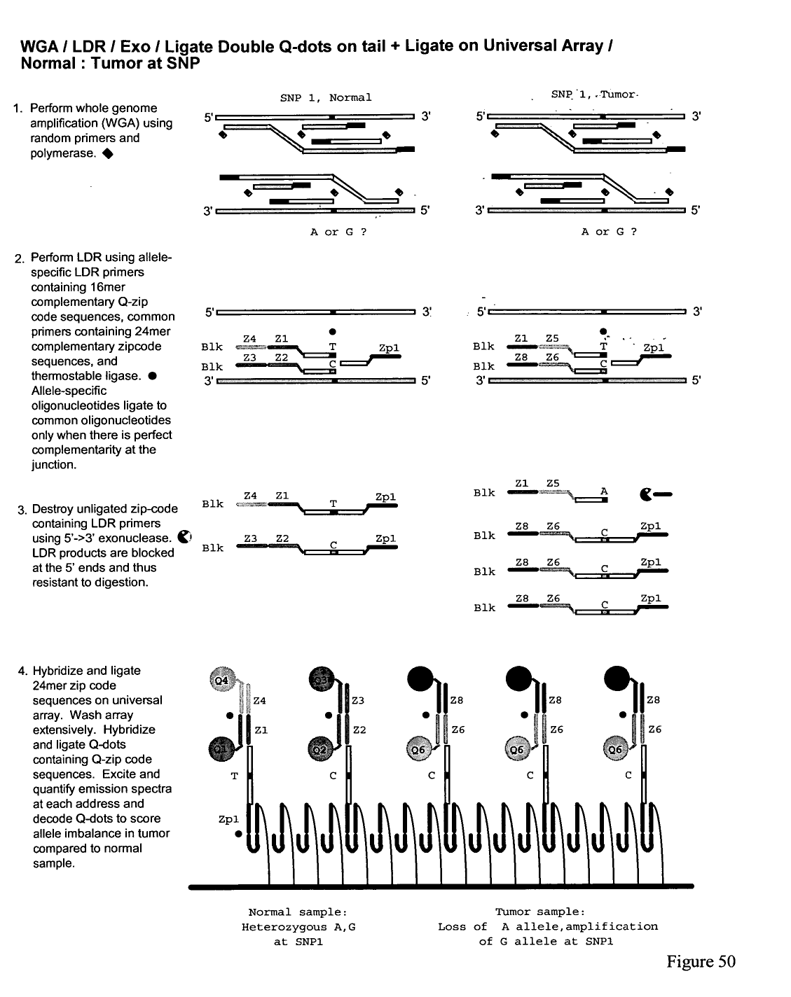

FIG. 50 is a schematic diagram of an LDR process, in accordance with the present invention, where whole genome amplification and ligated double Q-Zip portions containing Q-dots on a single tail are used with ligation to universal array capture to score allele imbalance in a tumor sample compared with a normal sample. FIG. 50 is similar to FIG. 49, but with the addition of ligating the captured addressable array-specific portions of their respective capture probes on the array. In order to allow ligation, the 3′ end of the addressable array-specific portion needs to remain unblocked, and, consequently, only a 5′→3′ exonuclease is used to destroy unligated versions of this probe. Alternatively, the 3′ end of the addressable array-specific portion may be synthesized with a thiophosphate group or other modification that inhibits 3′→5′ exonuclease activity, but not ligation. After ligation of ligation products to the array, unligated oligonucleotide probes containing complementary Q-Zip portions are removed by a stringent wash step. Subsequently, adjacent Q-dot containing adjacent Q-Zip portions are ligated in the last step.

The detection results in FIG. 50 show that at SNP1 the normal sample is heterozygous A, G and the tumor sample demonstrates a loss of the A allele and amplification of the G allele. In FIG. 50, the ratio of ligated Q-Zip portions containing Q-dots (Q1,Q5)/(Q4,Q1) at address Zp1 is about zero, indicating a loss of the A allele in the tumor. The ratio of ligated Q-Zip portions containing Q-dots (Q8,Q6)/(Q3,Q2) at address Zp1 is about three, indicating an approximately three-fold amplification of the G allele in the tumor.

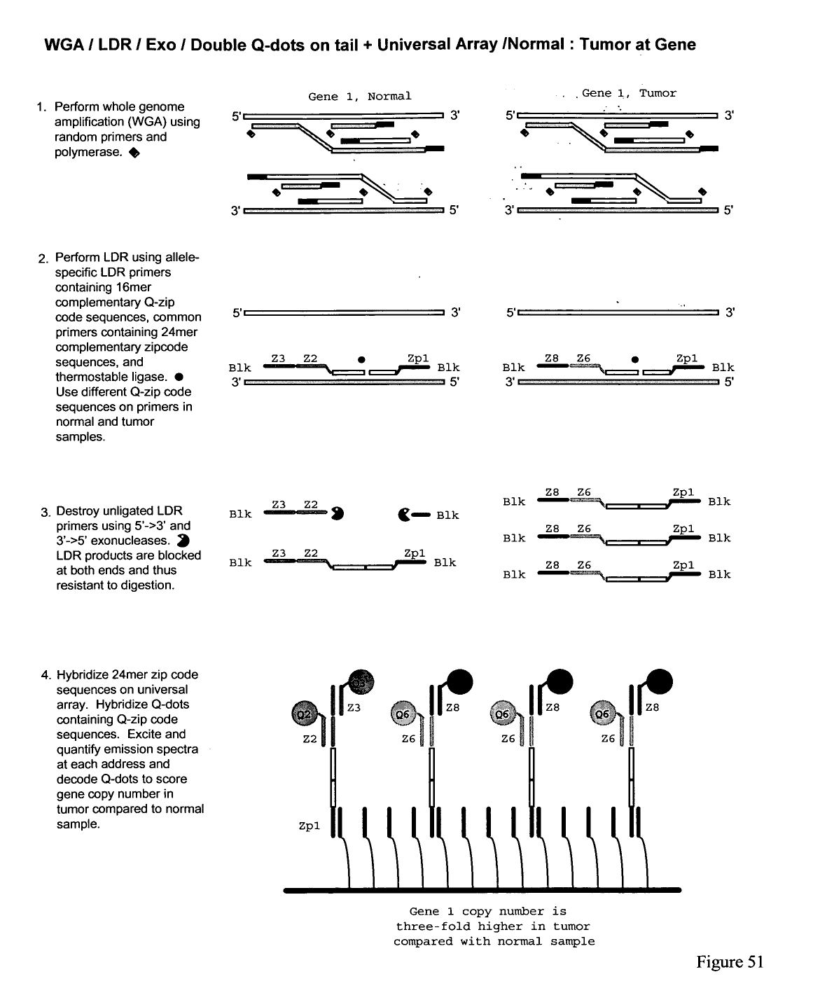

FIG. 51 is a schematic diagram of an LDR process, in accordance with the present invention, where whole genome amplification and double Q-Zip portions containing Q-dots on a single tail are used with universal array capture to score gene copy number in a tumor sample compared with a normal sample. This figure is similar to FIG. 48, except that the Q-dot encryption is used to score gene copy number in a tumor sample compared with the normal sample. In the initial LDR reaction, a given gene is interrogated with an allele-specific encrypted oligonucleotide probes in the normal sample in a first tube. The same gene is interrogated with a different allele-specific encrypted oligonucleotide probe in the tumor sample in a second tube. Although multiple different genes are detected in each tube, for simplicity, only one gene is shown. The ligation products are combined in a single tube, and the exonuclease digestion and remaining procedure is the same as described for FIG. 48. Emission spectra at each address are quantified to decode the presence of specific Q-dots to score gene copy number in the tumor compared to the normal sample. The detection results in FIG. 51 show an approximately three-fold amplification of gene 1 in the tumor sample (indicated by a ratio of Q-Zip portions containing Q-dots (Q8,Q6)/(Q3,Q2) at address Zp1 of about three).

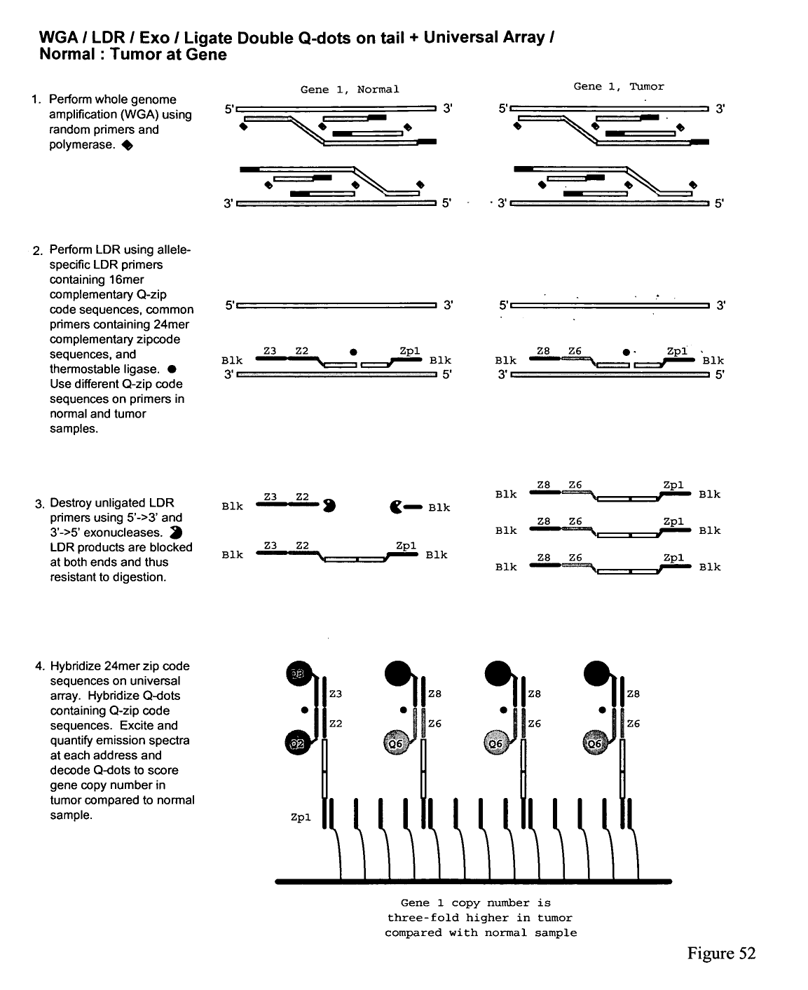

FIG. 52 is a schematic diagram of an LDR process, in accordance with the present invention, where whole genome amplification and ligated double Q-Zip portions containing Q-dots on a single tail are used with universal array capture to score gene copy number in a tumor sample compared with a normal sample. FIG. 52 is similar to FIG. 51, but with the addition of ligating adjacent Q-dot containing Q-Zip portions in the last step. The detection results in FIG. 51 show an approximately three-fold amplification of gene 1 in the tumor sample (indicated by a ratio of ligated Q-Zip portions containing Q-dots (Q8,Q6)/(Q3,Q2) at address Zp1 of about three).

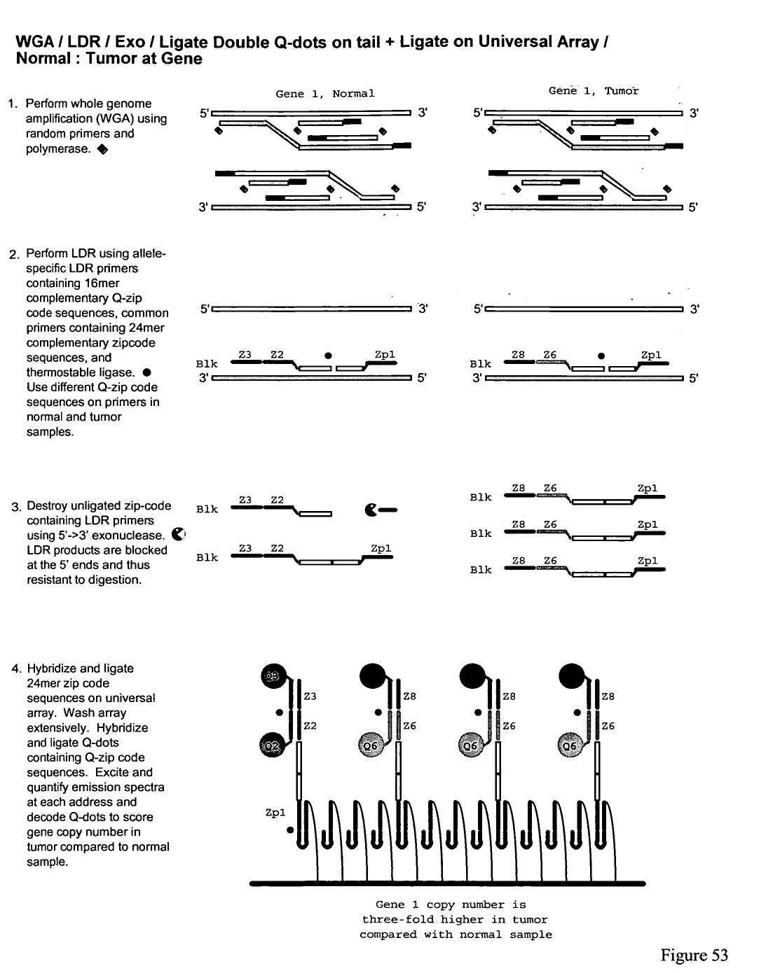

FIG. 53 is a schematic diagram of an LDR process, in accordance with the present invention, where whole genome amplification and ligated double Q-Zip portions containing Q-dots on a single tail are used with ligation to universal array capture to score gene copy number in a tumor sample compared with a normal sample. FIG. 53 is similar to FIG. 52, but with the addition of ligating the addressable array-specific portions to their respective capture oligonucleotide probes on the array. In order to allow ligation, the 3′ end of the addressable array-specific portion needs to remain unblocked, and, consequently, only a 5′→3′ exonuclease is used to destroy unligated versions of this oligonucleotide probe. Alternatively, the 3′ end of the addressable array-specific portion may be synthesized with a thiophosphate group or other modification that inhibits 3′→5′ exonuclease activity, but not ligation. After ligation of ligation products to the array, unligated oligonucleotide probes containing complementary Q-Zip portions are removed by a stringent wash step. Subsequently, Q-dot containing adjacent Q-Zip portions are ligated in the last step. The detection results in FIG. 53 show an approximately three-fold amplification of gene 1 in the tumor sample (indicated by a ratio of ligated Q-Zip portions containing Q-dots (Q8,Q6)/(Q3,Q2) at address Zp1 of about three).

In carrying out the ligase detection reaction/detection probe hybridization/detection scheme of the present invention, relative amounts of one or more of a plurality of target nucleic acid molecules in the test sample, differing by one or more single-base changes, insertions, deletions, or translocations, may be quantified by comparison with a reference sample having reference target nucleic acid molecules. This involves providing one or more secondary oligonucleotide probe sets, which differ from the primary oligonucleotide probe sets. Each of the secondary oligonucleotide probes sets are characterized by (a) a first oligonucleotide probe having a reference target-specific portion and (b) a second oligonucleotide probe having a reference target-specific portion. The oligonucleotide probes in a particular secondary oligonucleotide probe set are suitable for ligation together when hybridized adjacent to a corresponding reference target nucleic acid molecule. However, they have a mismatch which interferes with this ligation when hybridized to any other nucleic acid molecule present in the reference sample. One or both oligonucleotide probes in the secondary oligonucleotide probe set contain one or more detection oligonucleotide probe-specific portions or their complements so that each secondary oligonucleotide probe set contains a unique set of one or more detection oligonucleotide probe-specific portions or their complements. The reference sample, the one or more secondary oligonucleotide probe sets, and the ligase are blended to form a secondary ligase detection reaction mixture. The secondary ligase detection reaction mixture is then subjected to one or more ligase detection reaction cycles comprising a denaturation treatment and a hybridization treatment. In the denaturation treatment, any hybridized oligonucleotides are separated from reference target nucleic acid molecules. In the hybridization treatment, the oligonucleotide probe sets hybridize in a base-specific manner to their respective reference sample target nucleic acid molecule, if present in the sample, and ligate to one another to form a secondary ligation product. The secondary ligation product contains (a) the reference sample-specific portions and (b) the one or more detection oligonucleotide probe-specific portions or their complements. The secondary ligation product for each secondary oligonucleotide probe set is distinguishable from other nucleic acid molecules in the secondary ligase detection reaction mixture. The secondary oligonucleotide probe sets may hybridize to nucleic acid molecules in the reference sample other than their respective reference target nucleic acid molecules but do not ligate together due to the presence of one or more mismatches and individually separate during the denaturation treatment. The first and second ligase detection reaction mixtures are blended after subjecting them to one or more ligase detection reaction cycles and before capturing. As a result, the blended first and second ligase detection reaction mixtures are subjected to capturing, contacting, and detecting. The relative amounts of the reporter labels on the primary and secondary ligation products are then compared. This provides a quantitative measure of the relative level of the one or more target nucleic acid molecules in the test sample compared with the reference sample based on each different labeled ligation product having a unique encryption code with a different pattern of detectable emission spectra.

Ligase Detection Reaction/Polymerase Chain Reaction/Solid Support Capture/Ligase Detection Reaction/Detection Probe Hybridization/Detection Scheme

Another embodiment of the present invention relates to a method for identifying one or more target nucleic acid molecules differing by one or more single-base changes, insertions, deletions, or translocations. This method includes ligase detection reaction, polymerase chain reaction, solid support capture, ligase detection reaction, detection probe hybridization, and detection.

The ligase detection reaction phase involves providing a test sample potentially containing one or more target nucleic acid molecules. One or more primary oligonucleotide probe sets, each set characterized by (a) a first oligonucleotide probe, having a target-specific portion and a 5′ upstream portion containing a translational oligonucleotide portion and (b) a second oligonucleotide probe, having a target-specific portion, and a 3′ downstream primer-specific portion are provided. The oligonucleotide probes in a particular primary oligonucleotide probe set are suitable for ligation together when hybridized to a corresponding target nucleic acid molecule, but have a mismatch which interferes with this ligation when hybridized to any other nucleic acid molecule present in the test sample. The test sample, the one or more primary oligonucleotide probe sets, and a ligase are blended to form a primary ligase detection reaction mixture. The primary ligase detection reaction mixture is subjected to one or more ligase detection reaction cycles, as fully described above. These cycles each include a denaturation treatment and a hybridization treatment. In the denaturation treatment, any hybridized oligonucleotide probes are separated from the target nucleic acid molecules. In the hybridization treatment, the primary oligonucleotide probe sets hybridize in a base-specific manner to their respective target nucleic acid molecules, if present in the test sample, and ligate to one another to form a primary ligation product-containing (a) the 5′ upstream translational oligonucleotide portion, (b) the target-specific portions, and (c) the 3′ downstream primer-specific portion. The primary ligation product for each primary oligonucleotide probe set is distinguishable from other nucleic acids in the ligase detection reaction mixture. The primary oligonucleotide probe sets may hybridize to nucleic acid molecules in the test sample other than their respective target nucleic acid molecules but do not ligate together due to the presence of one or more mismatches and individually separate during the denaturation treatment.

In the polymerase chain reaction phase, a downstream primer complementary to the 3′ downstream primer-specific portion of the primary ligation product is provided. The primary ligation product is blended with the downstream primer and a polymerase to form a polymerase chain reaction mixture. The polymerase chain reaction mixture is subjected to one or more polymerase chain reaction cycles. As described above, the polymerase chain reaction comprising a denaturation treatment, where hybridized nucleic acid molecules are separated, a hybridization treatment, where the primer hybridizes to its complementary 3′ downstream primer-specific portion of the primary ligation product, and an extension treatment, where the hybridized primers are extended to form extension products complementary to the primary ligation product.

In the solid support capture phase, as described above, the extension product is captured on one or more solid supports, so that the extension product may be distinguished individually.

In the second ligase detection reaction phase, as described above, one or more secondary oligonucleotide probe sets, each set characterized by (a) a first oligonucleotide probe, having a translational oligonucleotide portion and a 5′ upstream portion complementary to one or more detection oligonucleotide probe-specific portions and (b) a second oligonucleotide probe, having a target portion, and a 3′ downstream portion complementary to one or more detection oligonucleotide probe-specific portions are provided. The oligonucleotide probes in a particular secondary oligonucleotide probe set are suitable for ligation together when hybridized to a corresponding captured primary extension product, but have a mismatch which interferes with this ligation when hybridized to any other nucleic acid molecule. The captured extension product, the one or more secondary oligonucleotide probe sets, and the ligase are blended to form a second ligase detection reaction mixture. The second ligase detection reaction mixture is subjected to one ligase detection reaction cycle. This cycle comprises a denaturation treatment, where any hybridized oligonucleotides are separated from the captured extension product, and a hybridization treatment, where the secondary oligonucleotide probe sets hybridize in a base-specific manner to their respective captured extension products, if present, and ligate to one another to form a secondary ligation product. The secondary ligation product contains (a) the 5 upstream portion comprising one or more detection oligonucleotide probe-specific portions, (b) the upstream translational oligonucleotide portion connected to the target portion, and (c) the 3′ downstream portion comprising one or more detection oligonucleotide probe-specific portions. The secondary ligation products for each secondary oligonucleotide probe set are distinguishable from other nucleic acids in the second ligase detection reaction mixture. The one or more secondary oligonucleotide probe sets may hybridize to nucleic acid molecules in the sample other than their respective captured extension products but do not ligate together due to the presence of one or more mismatches and individually separate during the denaturation treatment by heating to above the temperature at which each translational oligonucleotide portion melts, but below a temperature at which each secondary ligation product melts. As a result, each secondary ligation product remains hybridized to the captured extension product as a complex.

In the detection probe hybridization phase, as described above, detection oligonucleotide probes which bind to the detection oligonucleotide probe-specific portions of the complex are provided. Each detection oligonucleotide probe has a reporter label, thereby providing each complex containing a secondary ligation product with a unique detectable encryption code. The complex and the detection oligonucleotide probes are contacted under conditions effective to permit hybridization of the detection oligonucleotide probes to the complex so that a labeled complex is formed.

In the detection phase, as described above, the reporter label(s) on the complex are detected, thereby indicating the presence of one or more target nucleic acid molecule in the test sample. As a result, nucleic acid molecules differing by one or more single-base changes, insertions, deletions, or translocations are discriminated from one another during the primary and secondary ligase detection reactions and the discriminated nucleic acid molecules are detected as a result of each different labeled complex having a unique encryption code with a different pattern of detectable emission spectra.

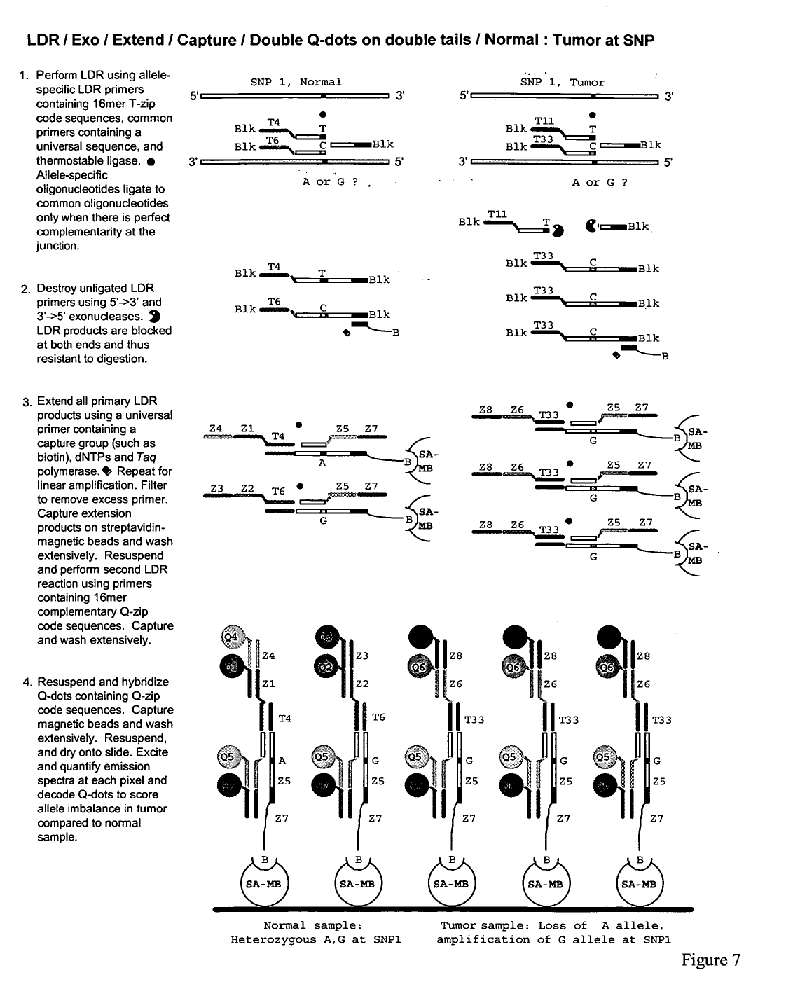

Examples of the ligase chain reaction, polymerase chain reaction, solid support capture, ligase detection reaction, detection probe hybridization, and detection scheme are shown in FIGS. 7-10.

FIG. 7 is a schematic diagram of an LDR process, in accordance with the present invention, where double Q-Zip portions containg Q-dots on double tails are used with microbead capture and a polymerase extension step to score allele imbalance in a tumor sample compared with a normal sample. LDR is performed using allele-specific oligonucleotide probes containing translation oligonucleotide portions (“T-Zip portions”), common oligonucleotide probes containing a universal sequence, and thermostable ligase. Allele-specific oligonucleotide probes containing T-Zip portions T4 and T6 discriminate A and G, respectively, on the target 3′→5′ nucleotide sequence of the normal sample. Allele-specific oligonucleotide probes containing T-Zip portions T 11 and T33 discriminate A and G, respectively, on the target 3′→5′ nucleotide sequence of the tumor sample. Allele-specific oligonucleotide probes ligate to common oligonucleotide probes only when there is perfect complementarity at the junction. Subsequently, unligated oligonucleotide probes are destroyed using 5′→3′ and 3′→5′ exonucleases. Ligation products are blocked at both ends and are thus resistant to digestion. These products may be linearly amplified by extending the primary ligation products using a universal primer containing a capture group (e.g., biotin), and Taq polymerase. The remaining biotin-containing primers are removed using filtration or size-exclusion columns. Extension products are then captured on streptavidin-magnetic beads and washed extensively. Magnetic beads containing the extension products are resuspended and a second LDR reaction is performed, using T-zip portion specific and target-specific oligonucleotide probes containing 16mer complementary Q-Zip portion. Magnetic beads are captured, washed extensively, resuspended, and hybridized with Q-dot containing Q-Zip portions. After capture and extensive washing, the magnetic beads are resuspended and dried onto a slide. Emission spectra at each pixel are quantified to decode the presence of specific Q-dots, to score allele imbalance in the tumor compared to the normal sample.

The detection results in FIG. 7 show that at SNP1 the normal sample is heterozygous A, G and the tumor sample demonstrates a loss of the A allele and amplification of the G allele. In FIG. 7, the ratio of hybridized Q-dot containing Q-Zip portions (Q1,Q5,Q5,Q7)/(Q4,Q1,Q5,Q7) is about zero, indicating loss of the A allele in the tumor. The ratio of hybridized Q-dot containing Q-Zip portions (Q8,Q6,Q5,Q7)/(Q3,Q2,Q5,Q7) is about three, indicating an approximately three-fold amplification of the G allele in the tumor.

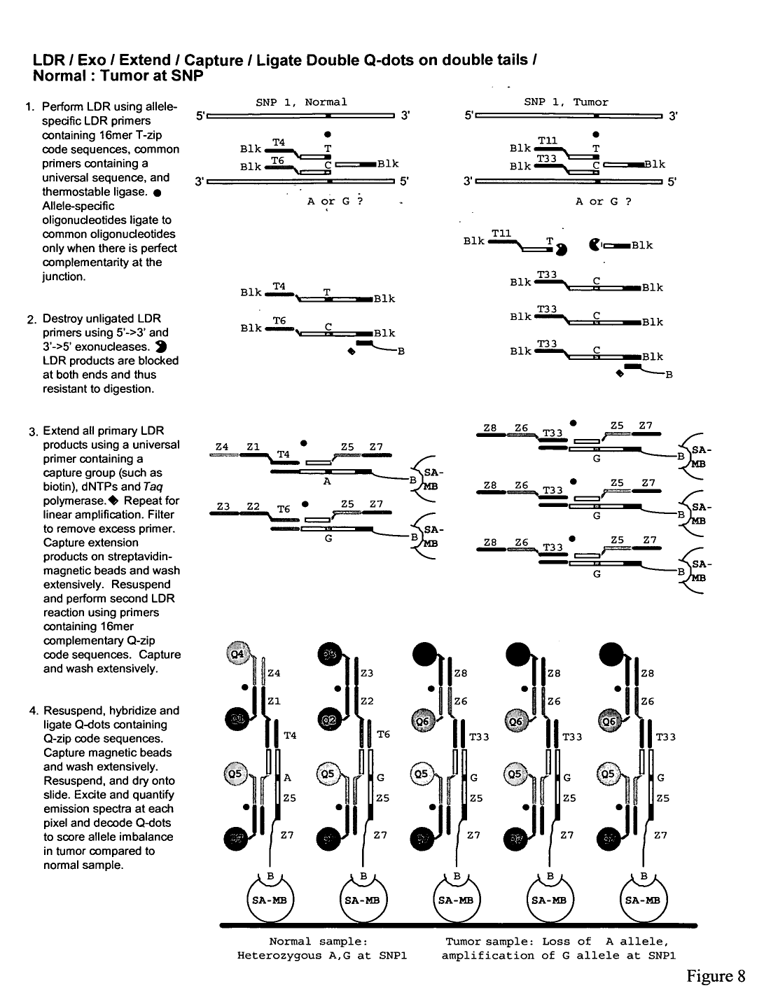

FIG. 8 is a schematic diagram of an LDR process, in accordance with the present invention, where ligated double Q-Zip portions containing Q-dots on double tails are used with microbead capture and a polymerase extension step to score allele imbalance in a tumor sample compared with a normal sample. FIG. 8 is similar to FIG. 7, but with the addition of ligating adjacent Q-Zip portions containing Q-dots.

The detection results in FIG. 8 show that at SNP1 the normal sample is heterozygous A, G and the tumor sample demonstrates a loss of the A allele and amplification of the G allele. In FIG. 8, the ratio of hybridized ligated Q-Zip portions containing Q-dots (Q1,Q5,Q5,Q7)/(Q4,Q1,Q5,Q7) is about zero, indicating loss of the A allele in the tumor. The ratio of hybridized ligated Q-Zip portions containing Q-dots (Q8,Q6,Q5,Q7)/(Q3,Q2,Q5,Q7) is about three, indicating an approximately three-fold amplification of the G allele in the tumor.

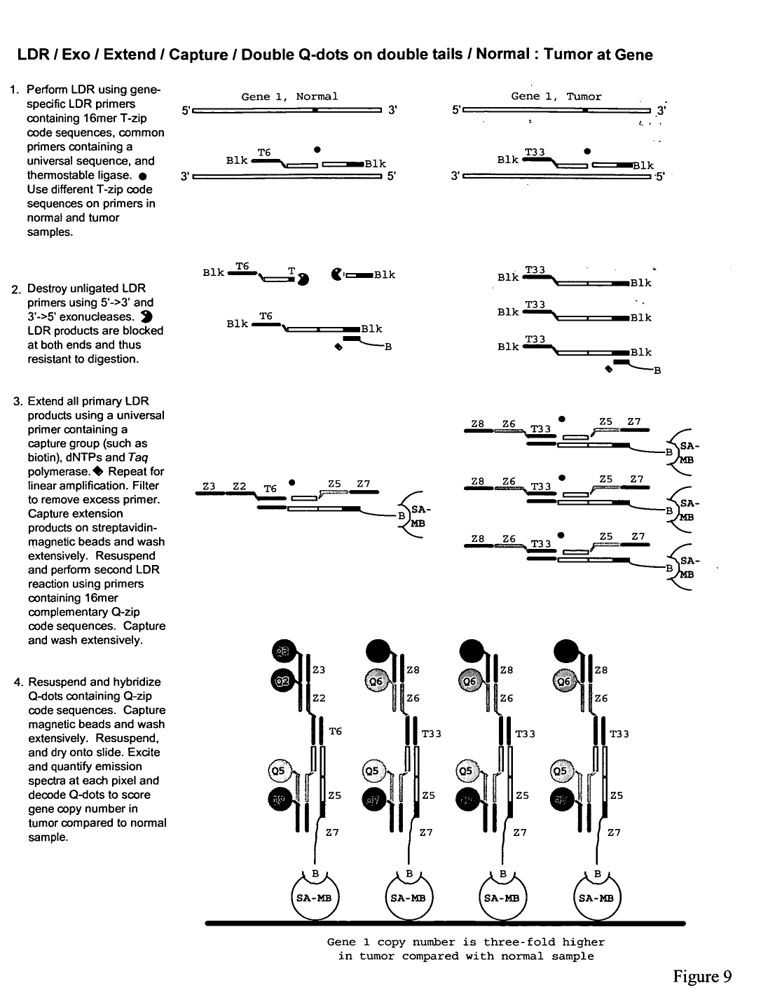

FIG. 9 is a schematic diagram of an LDR process, in accordance with the present invention, where double Q-Zip portions containing Q-dots on double tails are used with microbead capture and a polymerase extension step to score gene copy number in a tumor sample compared with a normal sample. FIG. 9 is similar to FIG. 7, except the Q-dot encryption is used to score gene copy number in a tumor sample compared with the normal sample. In the initial LDR reaction, a given gene is interrogated with a gene-specific encrypted oligonucleotide probes, containing a T-Zip portion in the normal sample in a first tube. The same gene is interrogated with a different gene-specific encrypted oligonucleotide probes, containing a different T-Zip portion in the tumor sample in a second tube. Although multiple different genes are detected in each tube, for simplicity only one gene is shown. The ligation products are combined in a single tube and the exonuclease digestion and subsequent procedure are the same as described in FIG. 7. Emission spectra at each pixel are quantified to decode the presence of specific Q-Zip portions containing Q-dots, to score gene copy number in the tumor compared to the normal sample. The detection results in FIG. 9 show an approximately three-fold amplification of gene 1 in the tumor sample (indicated by a ratio of hybridized Q-Zp portions containing Q-dots (Q8,Q6,Q5,Q7)/(Q3,Q2,Q5,Q7) of about three).

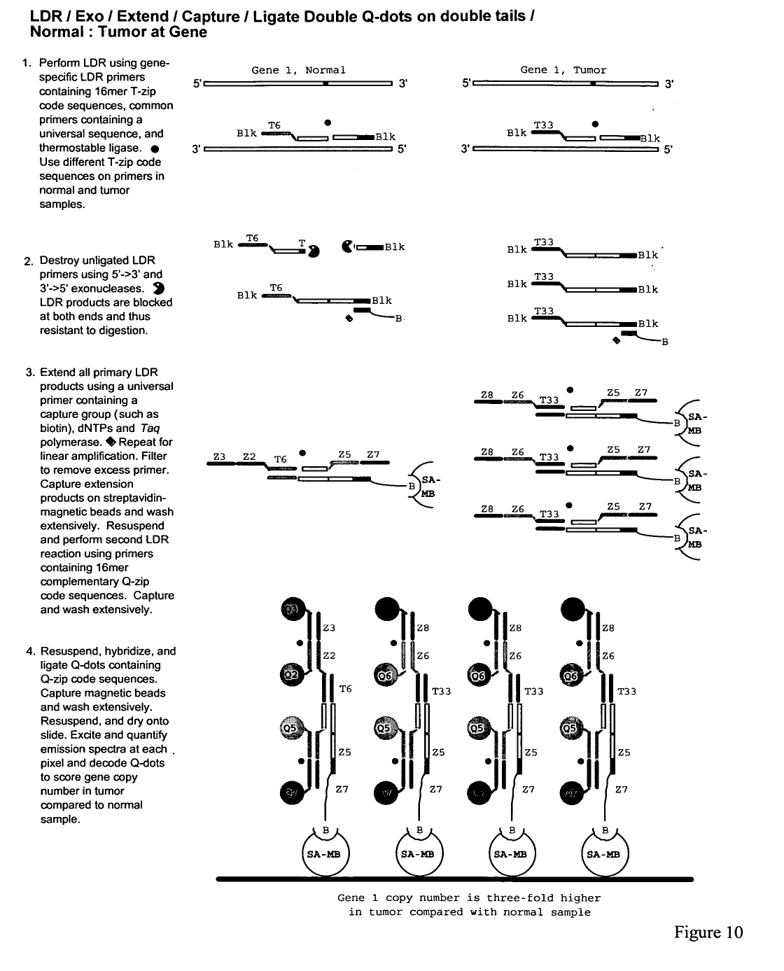

FIG. 10 is a schematic diagram of an LDR process, in accordance with the present invention, where ligated double Q-Zip portions containing Q-dots on double tails are used with microbead capture and a polymerase extension step to score gene copy number in a tumor sample compared with a normal sample. FIG. 10 is similar to FIG. 9, but with the addition of ligating adjacent Q-Zip portions containing Q-dots in the last step. The detection results in FIG. 10 show an approximately three-fold amplification of gene 1 in the tumor sample (indicated by a ratio of hybridized ligated Q-Zip portions containing Q-dots (Q8,Q6,Q5Q7)/(Q3Q2,Q5Q7) of about three).

In carrying out the ligase chain reaction, polymerase extension, solid support capture, ligase detection reaction, detection probe hybridization, and detection scheme of the present invention, relative amounts of one or more of a plurality of target nucleic acid molecules in the test sample, differing by one or more single-base changes, insertions, deletions, or translocations, may be quantified by comparison with a reference sample.

This involves providing one or more tertiary oligonucleotide probe sets, which differ from the primary oligonucleotide probe sets. Each of the tertiary oligonucleotide probes sets are characterized by (a) a first oligonucleotide probe, having a reference target-specific portion and a 5′ upstream portion containing a translational oligonucleotide probe-specific portion and (b) a second oligonucleotide probe, having a reference target-specific portion, and a 3′ downstream primer specific portion. The oligonucleotide probes in a particular tertiary oligonucleotide probe set are suitable for ligation together when hybridized on a corresponding reference target nucleic acid molecule, but have a mismatch which interferes with this ligation when hybridized to any other nucleic acid molecule present in the reference sample.

The reference sample, the one or more tertiary oligonucleotide probe sets, and the ligase are blended to form a tertiary ligase detection reaction mixture. The tertiary ligase detection reaction mixture is then subjected to one or more ligase detection reaction cycles comprising a denaturation treatment and a hybridization treatment. In the denaturation treatment, any hybridized oligonucleotides are separated from the reference target nucleic acid molecules. In the hybridization treatment, the oligonucleotide probe sets hybridize in a base-specific manner to their respective reference target nucleic acid molecule, if present in the sample, and ligate to one another to form a tertiary ligation product containing (a) the 5′ upstream translational oligonucleotide-specific portion, (b) the target-specific portions, and (c) the 3′ downstream primer specific portion. The tertiary ligation product for each tertiary oligonucleotide probe set is distinguishable from other nucleic acid molecules in the tertiary ligase detection reaction mixture. The tertiary oligonucleotide probe sets may hybridize to nucleic acid molecules in the sample other than their respective reference target nucleic acid molecules but do not ligate together due to the presence of one or more mismatches and individually separate during the denaturation treatment.

The primary and tertiary ligase detection reaction mixtures are blended after subjecting them to one or more ligase detection reaction cycles and before subjecting the polymerase chain reaction mixture to one or more polymerase chain reaction cycles. The blended primary and tertiary ligase detection reaction mixtures are subjected to one or more polymerase chain reaction cycles, capturing, subjecting the secondary ligase detection reaction mixture to one ligase detection reaction cycles, contacting the complex and the detection oligonucleotide probes, and detecting the reporter labels on the complex. The relative amounts of the reporter label on the complexes is then compared, to provide a quantitative measure of the relative level of the one or more target nucleic acid molecules in the test sample compared with the reference sample as a result of each different labeled complex having a unique encryption code with a different pattern of detectable emission spectra.

Ligase Detection Reaction, Solid Support Capture, Detection Probe Hybridization, and Detection Scheme

Another aspect of the present invention is directed to a method for identifying one or more target nucleic acid molecules, differing by one or more single-base changes, insertions, deletions, or translocations, in a plurality of nucleic acid molecules or identifying one or more target mRNA molecules differing by one or more splice site variations in a plurality of mRNA molecules. This method includes ligase detection reaction, solid support capture, detection probe hybridization, and detection.

The ligase detection reaction phase, as described above, involves providing a test sample potentially containing one or more target nucleic acid molecules differing by one or more single-base changes, insertions, deletions, or translocations, in a plurality of nucleic acid molecules or one or more target mRNA molecules differing by one or more splice site variations in a plurality of mRNA molecules.

One or more primary oligonucleotide probe sets are provided. Each set is characterized by (a) a first oligonucleotide probe, having one or more detection oligonucleotide probe-specific portions or their complements and a target-specific portion and (b) a second oligonucleotide probe, having a target-specific portion and an addressable array-specific portion or its complement. The oligonucleotide probes in a particular set are suitable for ligation together when hybridized to a corresponding target nucleic acid molecule or target mRNA molecule, but have a mismatch which interferes with this ligation when hybridized to any other nucleic acid molecule or mRNA molecule present in the sample. As a result, each probe set contains a unique combination of detection oligonucleotide probe-specific portions and addressable array-specific portions or their complements.

The sample, the one or more oligonucleotide probe sets, and a ligase are blended to form a primary ligase detection reaction mixture. The primary ligase detection reaction mixture is then subjected to one or more primary ligase detection reaction cycles. These cycles include a denaturation treatment and a hybridization treatment. In the denaturation treatment, any hybridized oligonucleotides are separated from the target nucleic acid molecules or target mRNA molecules. During the hybridization treatment, the one or more oligonucleotide probe sets hybridize in a base-specific manner to their respective target nucleic acid molecules or target mRNA molecules, if present in the sample, and ligate to one another to form a primary ligation product containing (a) the one or more detection oligonucleotide probe-specific portions or their complements, (b) the target-specific portions, and (c) the addressable array-specific portion or its complement. The primary ligation product for each one or more oligonucleotide probe set is distinguished from other nucleic acid molecules or mRNA molecules in the primary ligase detection reaction mixture by virtue of their containing a unique combination of detection oligonucleotide probe-specific portions and addressable array-specific portions or their complements. The primary oligonucleotide probe sets may hybridize to nucleic acid molecules or mRNA molecules in the sample other than their respective target nucleic acid molecules or target mRNA molecules but do not ligate together due to the presence of one or more mismatches and individually separate during the denaturation treatment.

In the solid support capture phase, as described above, a solid support with capture oligonucleotide probes immobilized at different sites is provided. The capture oligonucleotide probes have nucleotide sequences complementary to the addressable array-specific portions or their complements. The primary ligation products, copies of primary ligation products, or complements thereof are contacted with the solid support under conditions effective to hybridize the primary ligation products, copies of primary ligation products, or complements thereof to the capture oligonucleotide probes in a base-specific manner. As a result, the primary ligation products, copies of primary ligation products, or complements thereof are captured on the solid support at the site with the complementary capture oligonucleotide.

In the detection probe hybridization phase, as described bove, detection oligonucleotide probes are provided which bind to the detection oligonucleotide probe-specific portions of the captured primary ligation products, copies of primary ligation products, or complements thereof. Each detection oligonucleotide probe has a reporter label, so that each of the captured primary ligation products, copies of primary ligation products, or complements thereof have a unique detectable encryption code. The captured primary ligation products, copies of primary ligation products, or complements thereof are contacted with the detection oligonucleotide probes under conditions effective to permit hybridization of the detection oligonucleotide probes to the captured primary ligation products, copies of primary ligation products, or complements thereof so that labeled, captured primary ligation products, copies of primary ligation products, or complements thereof are formed.

In the detection phase, as described above, the reporter labels on the labeled, captured primary ligation products, copies of primary ligation products, or complements thereof are detected, thereby indicating the presence of one or more target nucleic acid molecules or target mRNA molecules in the sample. As a result, target nucleic acid molecules differing by one or more single-base changes, insertions, deletions, or translocations in a plurality of nucleic acid molecules or target mRNA molecules differing by one or more splice site variations in a plurality of mRNA molecules are discriminated from one another during the primary ligase detection reaction. The discriminated molecules are detected as a result of different labeled ligation products having encryption codes with a different pattern of detectable emission spectra, at different sites on the solid support.

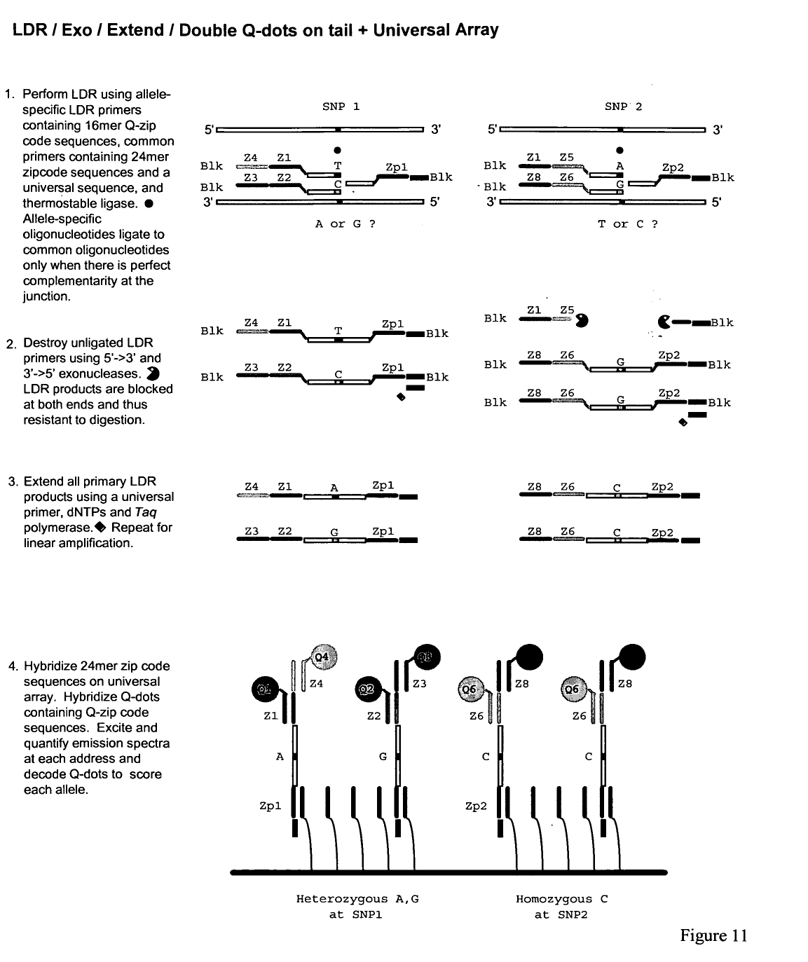

Examples of the ligase detection reaction, solid support capture, detection probe hybridization, and detection scheme are shown in FIGS. 11-16 and 54-62.

FIG. 11 is a schematic diagram of an LDR process, in accordance with the present invention, where double Q-Zip portions containing Q-dots on a single tail are used with universal array capture to distinguish single base differences in nucleic acid molecules. LDR is performed using allele-specific oligonucleotide probes containing 16mer complementary Q-Zip portions, common oligonucleotide probes containing 24mer addressable array-specific portions and a universal sequence, and thermostable ligase. Allele-specific addressable array-specific portions (Z4,Z1), (Z3,Z2), (Z1,Z5), and (Z8,Z6) discriminate A, G, T, and C, respectively, on the target 3′→5′ nucleotide sequence. Allele-specific oligonucleotide probes ligate to common oligonucleotide probes only when there is perfect complementarity at the junction. Subsequently, unligated oligonucleotide probes are destroyed using 5′→3′ and 3′→5′ exonucleases. Ligation products are blocked at both ends and are thus resistant to digestion. All primary ligation products are extended using a universal primer, dNTPs and Taq polymerase. The extension step is repeated for linear amplification of ligation products as necessary. Extension products are hybridized onto a universal array via the 24-mer addressable array-specific portions. Extension products on the universal array are hybridized with Q-dot containing Q-Zip portions. Emission spectra at each address are quantified to decode the presence of specific Q-dots, to score each allele.

The detection results in FIG. 11 show that the sample is heterozygous A, G at SNP1 and homozygous C at SNP2. The A allele is indicated by the presence of Q-Zip portions containing Q-dots (Q4,Q1) at address Zp1. The G allele is indicated by the presence of Q-Zip portions containing Q-dots (Q3,Q2) at address Zp1. The C allele is indicated by the presence of Q-Zip portions containing Q-dots (Q8,Q6) at address Zp2.

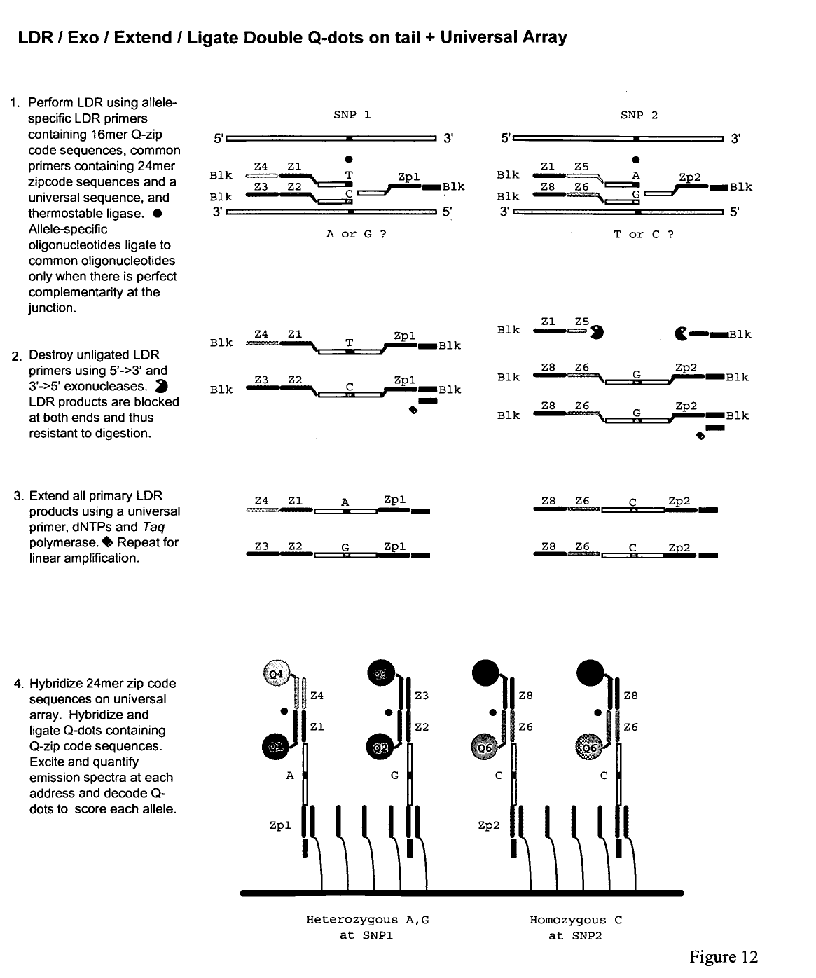

FIG. 12 is a schematic diagram of an LDR process, in accordance with the present invention, where ligated double Q-Zip portions containing Q-dots on a single tail are used with universal array capture to distinguish single base differences in nucleic acid molecules. FIG. 12 is similar to FIG. 11, but with the addition of ligating adjacent Q-Zip portions containing Q-dots in the last step.

The detection results in FIG. 12 show that the sample is heterozygous A, G at SNP1 and homozygous C at SNP2. The A allele is indicated by the presence of ligated Q-Zip portions containg Q-dots (Q4,Q1) at address Zp1. The G allele is indicated by the presence of ligated Q-Zip portions containing Q-dots (Q3,Q2) at address Zp1. The C allele is indicated by the presence of ligated Q-Zip portions containing Q-dots (Q8,Q6) at address Zp2.

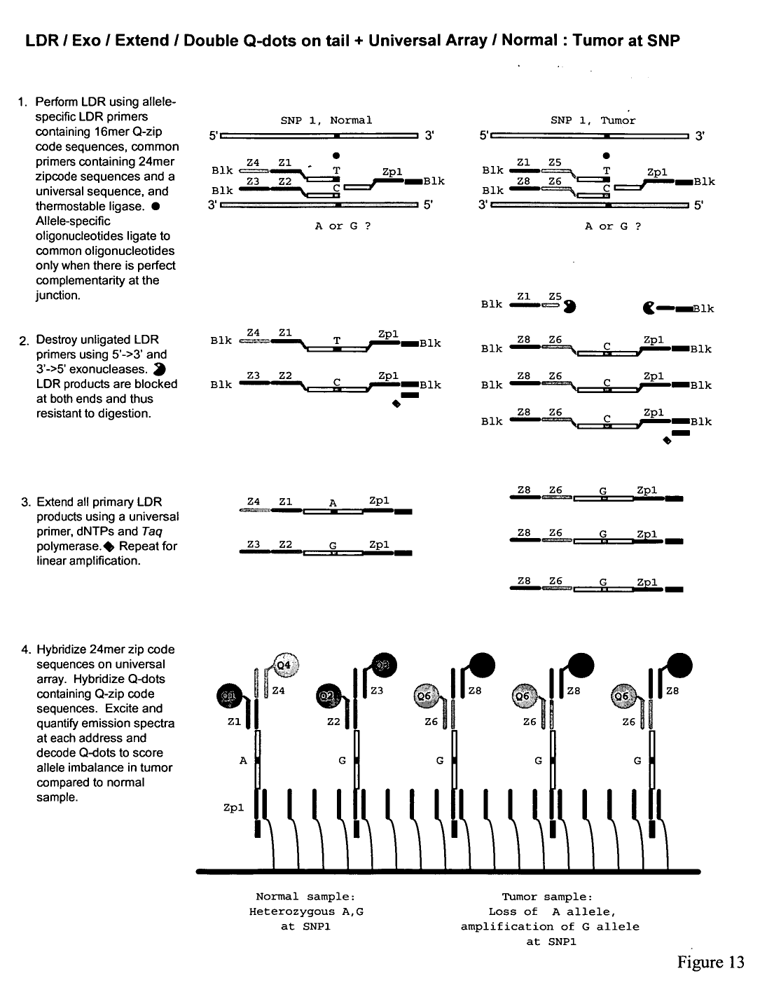

FIG. 13 is a schematic diagram of an LDR process, in accordance with the present invention, where double Q-Zip portions containing Q-dots on a single tail are used with universal array capture to score allele imbalance in a tumor sample compared with a normal sample. FIG. 13 is similar to FIG. 11, except the Q-dot encryption is used to score allele imbalance in a tumor sample compared with the normal sample. In the initial LDR reaction, a given single base difference is interrogated with two allele-specific encrypted oligonucleotide probes in the normal sample in a first tube. The same single base difference is interrogated with two different allele-specific encrypted oligonucleotide probes in the tumor sample in a second tube. Although multiple different single base differences are detected in each tube, for simplicity only SNP1 is shown. The ligation products are combined in a single tube and the exonuclease digestion and subsequent procedure are the same as described in FIG. 11. Emission spectra at each address are quantified to decode the presence of specific Q-dots, to score allele imbalance in the tumor compared to the normal sample.

The detection results in FIG. 13 show that at SNP1 the normal sample is heterozygous A, G and the tumor sample demonstrates a loss of the A allele and amplification of the G allele. In FIG. 13, the ratio of Q-Zip portions containing Q-dots (Q1,Q5)/(Q4,Q1) at address Zp1 is about zero, indicating loss of the A allele in the tumor. The ratio of Q-Zip portions containing Q-dots (Q8,Q6)/(Q3,Q2) at address Zp1 is about three, indicating an approximately three-fold amplification of the G allele in the tumor.

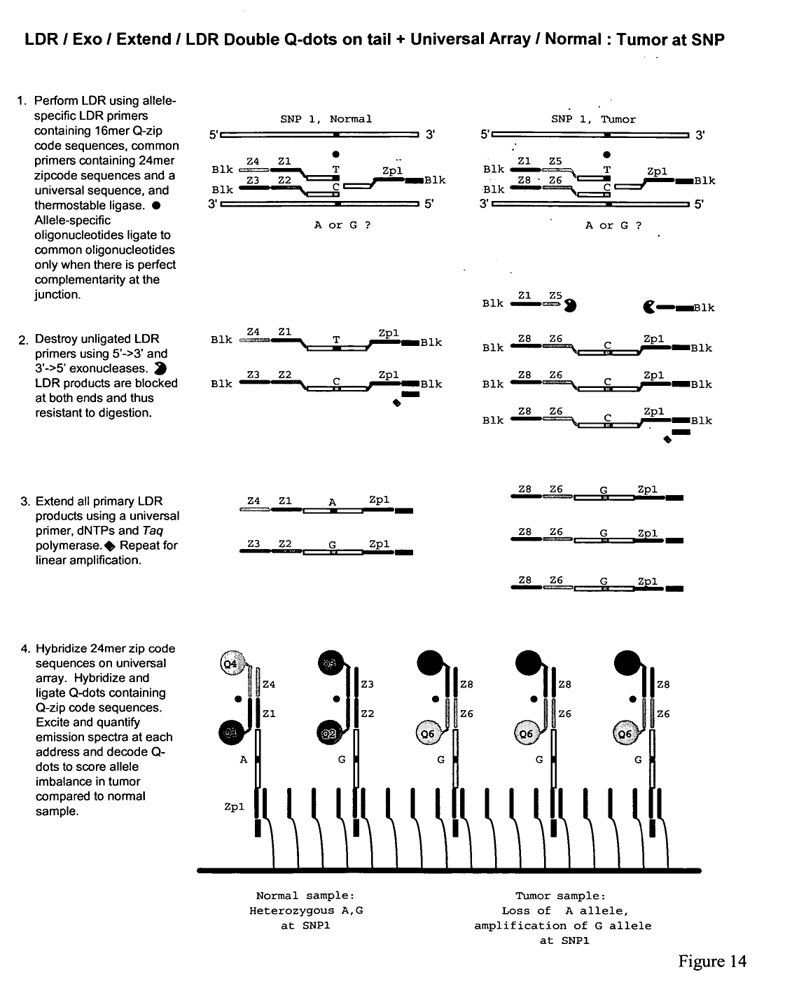

FIG. 14 is a schematic diagram of an LDR process, in accordance with the present invention, where ligated double Q-Zip portions containing Q-dots on a single tail are used with universal array capture to score allele imbalance in a tumor sample compared with a normal sample. FIG. 14 is similar to FIG. 13, but with the addition of ligating adjacent Q-Zip portions containing Q-dots in the last step.

The detection results in FIG. 14 show that at SNP1 the normal sample is heterozygous A, G and the tumor sample demonstrates a loss of the A allele and amplification of the G allele. In FIG. 14, the ratio of ligated Q-Zip portions containing Q-dots (Q1,Q5)/(Q4,Q1) at address Zp1 is about zero, indicating loss of the A allele in the tumor. The ratio of ligated Q-Zip portions containing Q-dots (Q8,Q6)/(Q3,Q2) at address Zp1 is about three, indicating an approximately three-fold amplification of the G allele in the tumor.

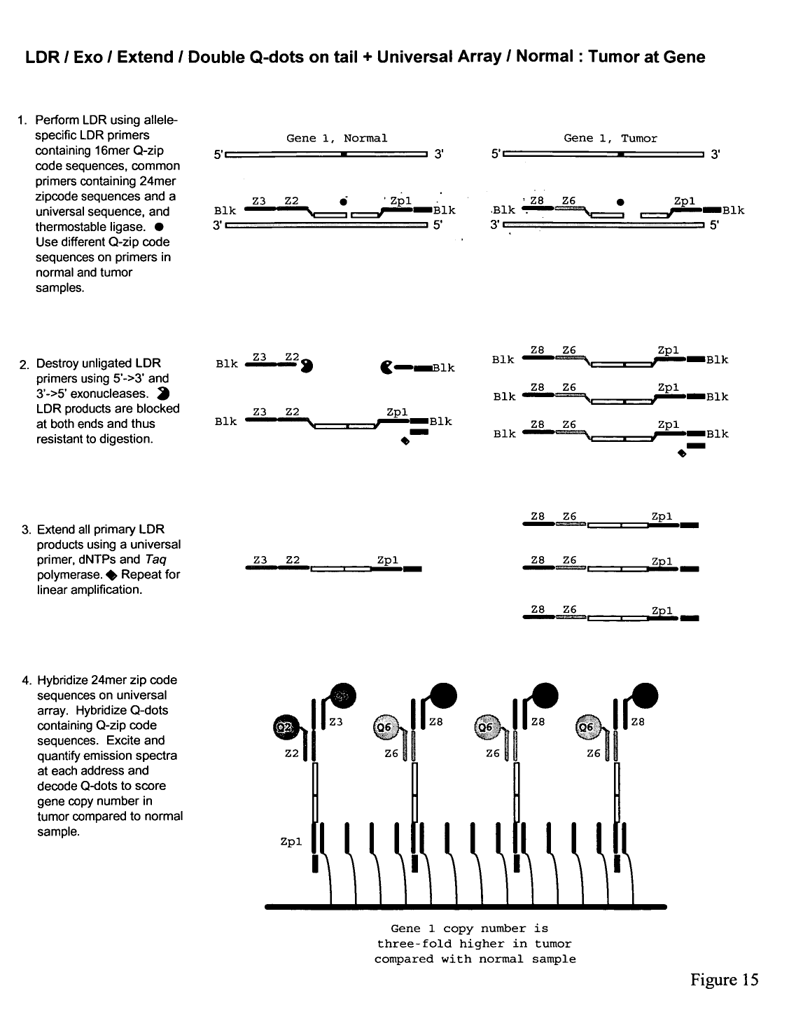

FIG. 15 is a schematic diagram of an LDR process, in accordance with the present invention, where double Q-Zip portions containing Q-dots on a single tail are used with universal array capture to score gene copy number in a tumor sample compared with a normal sample. FIG. 15 is similar to FIG. 11, except the Q-dot encryption is used to score gene copy number in a tumor sample compared with the normal sample. In the initial LDR reaction, a given gene is interrogated with an allele-specific encrypted oligonucleotide probe in the normal sample in a first tube. The same gene is interrogated with a different allele-specific encrypted oligonucleotide probe in the tumor sample in a second tube. Although multiple different genes are detected in each tube, for simplicity only one gene is shown. The ligation products are combined in a single tube and the exonuclease digestion and subsequent procedure are the same as described in FIG. 11. Emission spectra at each address are quantified to decode the presence of specific Q-dots, to score gene copy number in the tumor compared to the normal sample. The detection results in FIG. 15 show an approximately three-fold amplification of gene 1 in the tumor sample (indicated by a ratio of Q-Zip portions containing Q-dots (Q8,Q6)/(Q3,Q2) at address Zp1 of about three).

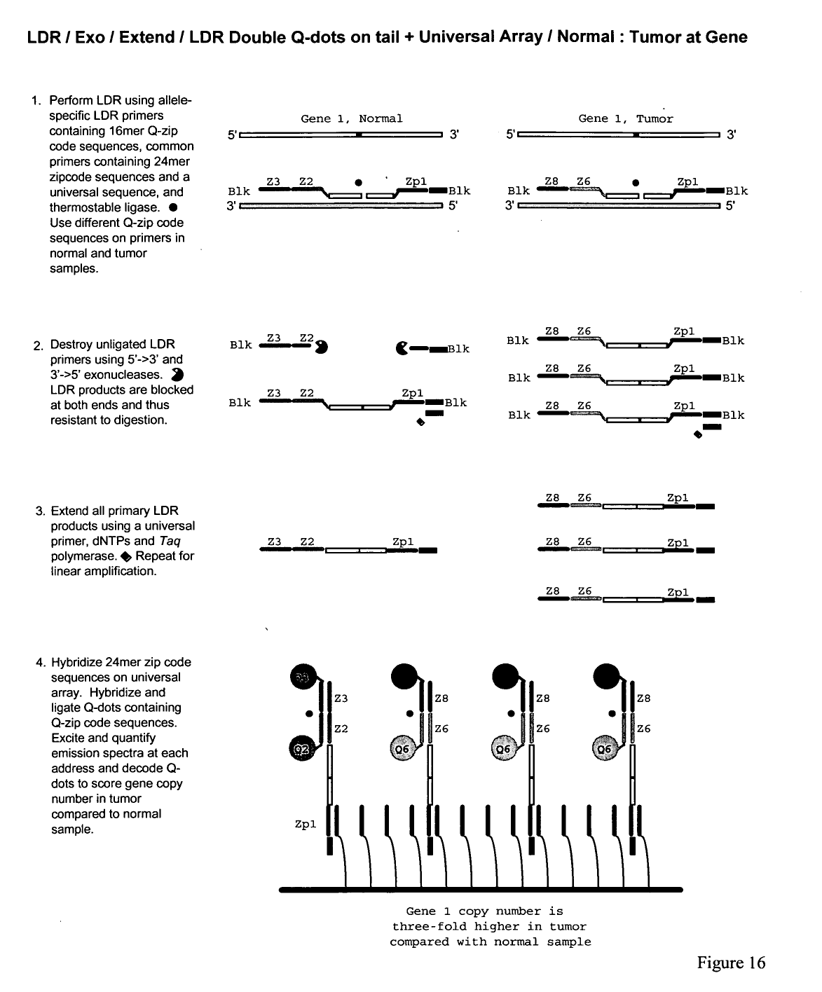

FIG. 16 is a schematic diagram of an LDR process, in accordance with the present invention, where ligated double Q-Zip portions containing Q-dots on a single tail are used with universal array capture to score gene copy number in a tumor sample compared with a normal sample. FIG. 16 is similar to FIG. 15, but with the addition of ligating adjacent Q-Zip portions containing Q-dots in the last step. The detection results in FIG. 16 show an approximately three-fold amplification of gene 1 in the tumor sample (indicated by a ratio of ligated Q-Zip portions containing Q-dots (Q8,Q6)/(Q3,Q2) at address Zp1 of about three).

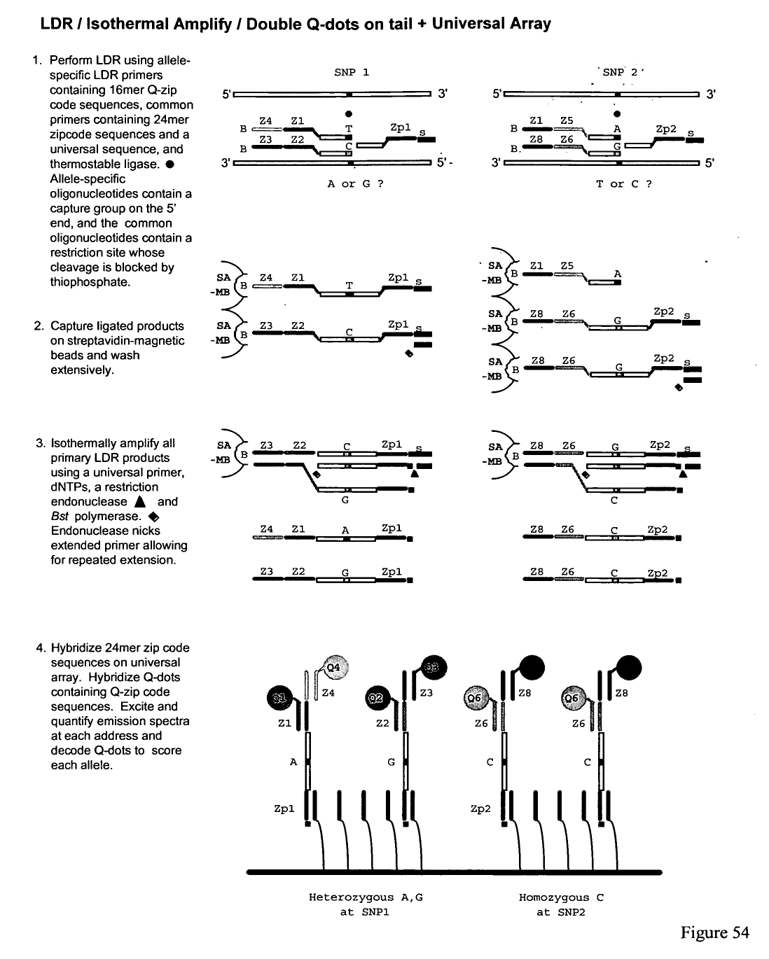

FIG. 54 is a schematic diagram of an LDR process, in accordance with the present invention, where isothermal amplification and double Q-Zip portions containing Q-dots on a single tail are used with universal array capture to distinguish single base differences in nucleic acid molecules. LDR is performed using allele-specific oligonucleotide probes containing 16mer Q-Zip portions, common oligonucleotide probes containing 24mer addressable array-specific portions and a universal sequence, and thermostable ligase. Allele-specific oligonucleotide probes contain a capture group on the 5′ end, and the common oligonucleotide probes contain a restriction site whose cleavage is blocked by a thiophosphate group at the scissile bond. Allele-specific addressable array-specific portions (Z4,Z1), (Z3,Z2), (Z1,Z5), and (Z8,Z6) discriminate A, G, T, and C, respectively, on the target 3′→5′ nucleotide sequence. Allele-specific oligonucleotide probes ligate to common oligonucleotide probes only when there is perfect complementarity at the junction. Subsequently, ligation products are captured on streptavidin-magnetic beads and washed extensively. All primary ligtion products are amplified isothermally using a universal primer, dNTPs, a restriction endonuclease, and Bst polymerase. The universal primer contains the restriction endonuclease site and hybridizes to the ligation products. Once Bst polymerase extends this primer, the restriction endonuclease nicks this strand while extension continues. The ligation product is not cleaved as the thiophosphate strand is resistant. However, the nicked primer becomes a substrate for a new polymerase to bind and extend, displacing the previous strand. This isothermal amplification allows hundreds to thousands or more copies to accumulate in a linear fashion. Extension products are hybridized onto a universal array via the 24-mer addressable array-specific portions in the ligation products and capture oligonucleotide probes on the solid support. Extension products on the universal array are hybridized with Q-dot containing Q-Zip portions. Emission spectra at each address are quantified to decode the presence of specific Q-dots, to score each allele.

The detection results in FIG. 54 show that the sample is heterozygous A, G at SNP1 and homozygous C at SNP2. The A allele is indicated by the presence of Q-Zip portions containing Q-dots (Q4,Q1) at address Zp1. The G allele is indicated by the presence of Q-Zip portions containing Q-dots (Q3,Q2) at address Zp1. The C allele is indicated by the presence of Q-Zip portions containing Q-dots (Q8,Q6) at address Zp2.

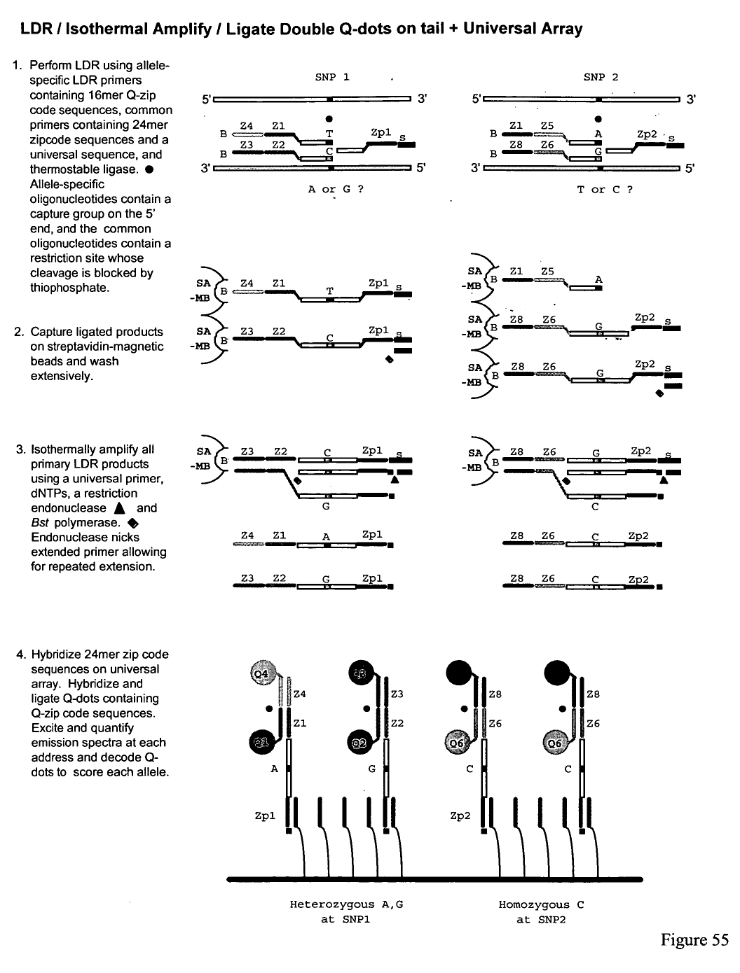

FIG. 55 is a schematic diagram of an LDR process, in accordance with the present invention, where isothermal amplification and ligated double Q-Zip portions containing Q-dots on a single tail are used with universal array capture to distinguish single base differences in nucleic acid molecules. FIG. 55 is similar to FIG. 54, but with the addition of ligating adjacent Q-dot containing Q-Zip portions in the last step.

The detection results in FIG. 55 show that the sample is heterozygous A, G at SNP1 and homozygous C at SNP2. The A allele is indicated by the presence of ligated Q-Zip portions containing Q-dots (Q4,Q1) at address Zp1. The G allele is indicated by the presence of ligated Q-Zip portions containing Q-dots (Q3,Q2) at address Zp1. The C allele is indicated by the presence of ligated Q-Zip portions containing Q-dots (Q8,Q6) at address Zp2.

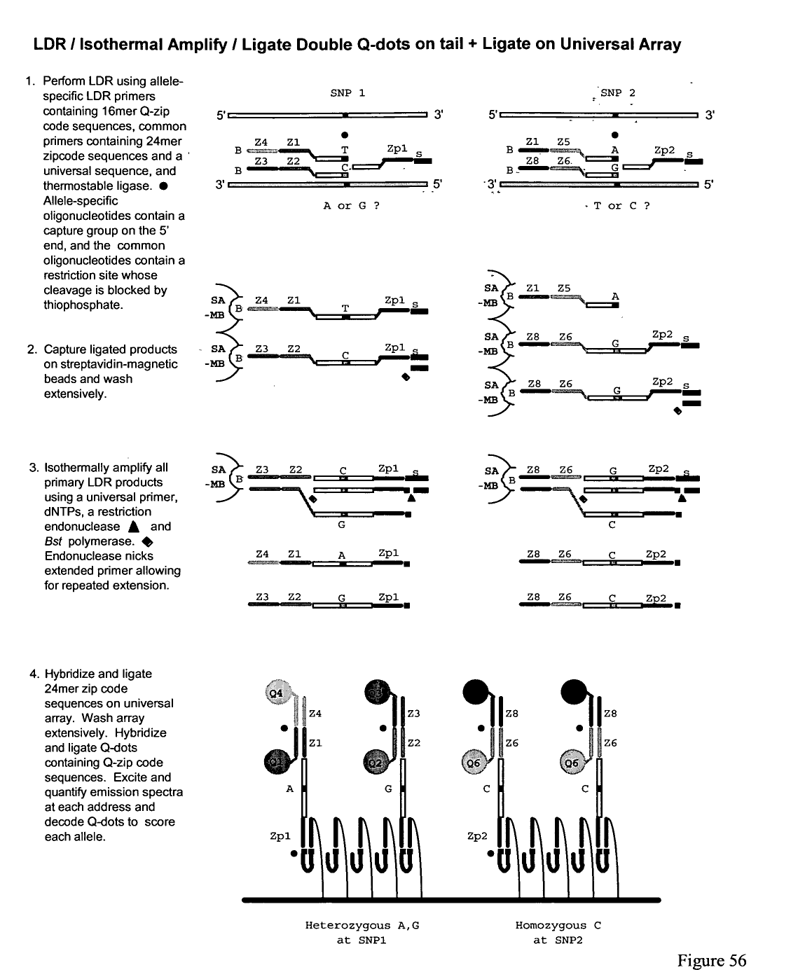

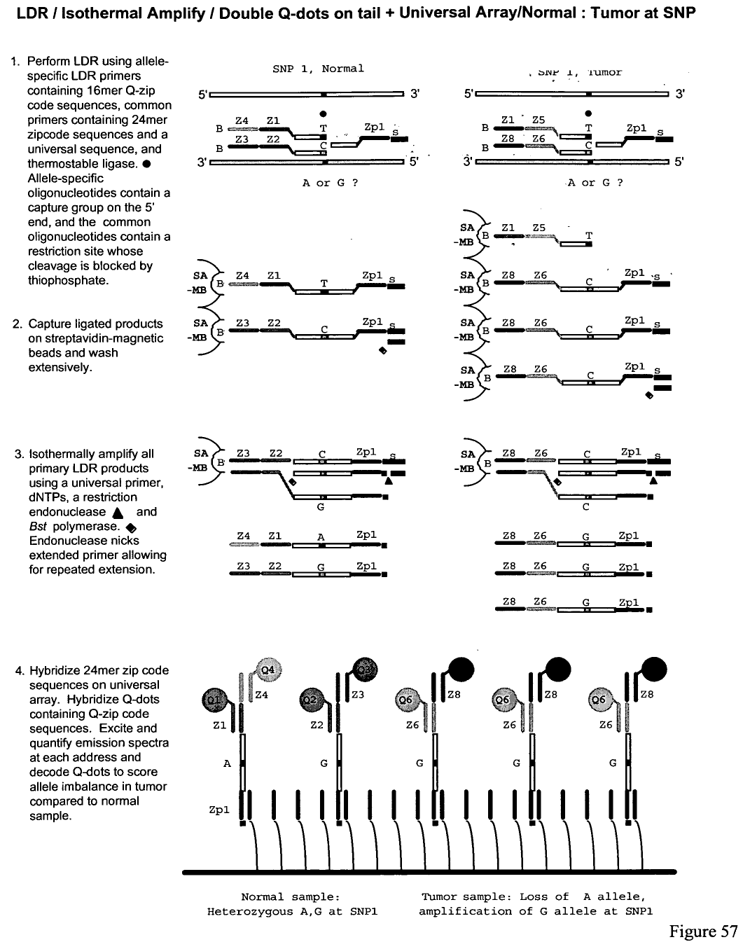

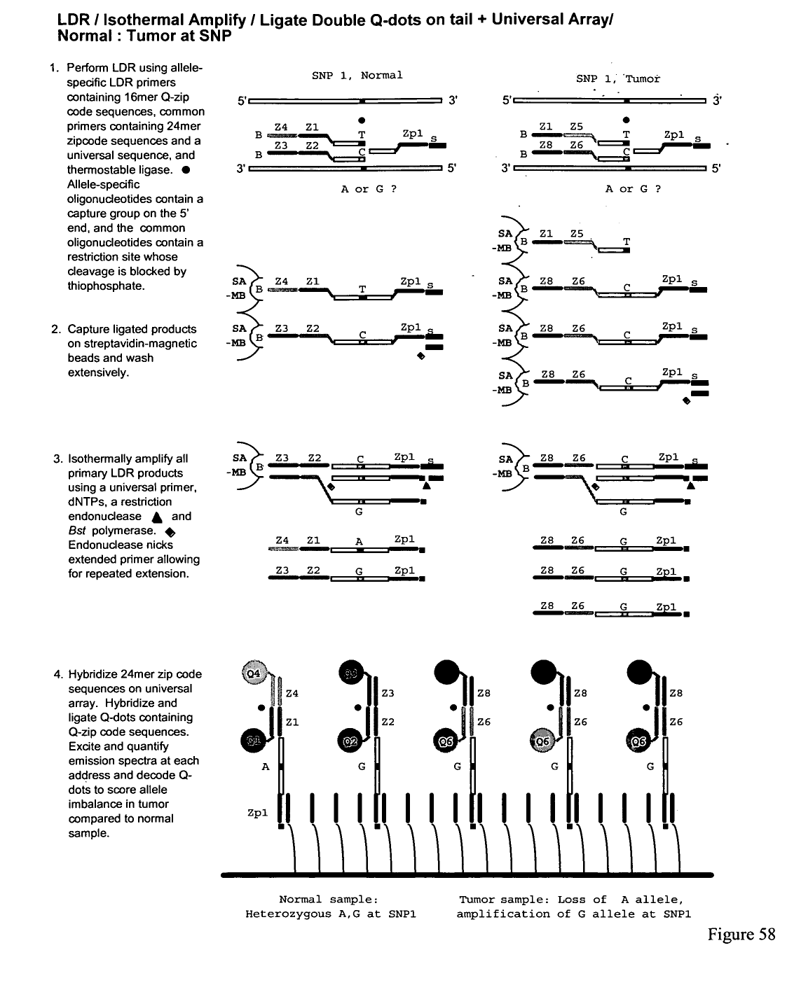

FIG. 56 is a schematic diagram of an LDR process, in accordance with the present invention, where isothermal amplification and ligated double Q-Zip portions containing Q-dots on a single tail are used with ligation to universal array capture to distinguish single base differences in nucleic acid molecules. FIG. 56 is similar to FIG. 55, but with the addition of ligating the captured ligation products to their respective capture probes of the array followed by a stringent wash step. Subsequently, adjacent Q-Zip portions containing Q-dots are ligated in the last step.

The detection results in FIG. 56 show that the sample is heterozygous A, G at SNP1 and homozygous C at SNP2. The A allele is indicated by the presence of ligated Q-Zip portions containing Q-dots (Q4,Q1) at address Zp1. The G allele is indicated by the presence of ligated Q-Zip portions containing Q-dots (Q3,Q2) at address Zp1. The C allele is indicated by the presence of ligated Q-Zip portions containing Q-dots (Q8,Q6) at address Zp2.

FIG. 57 is a schematic diagram of an LDR process, in accordance with the present invention, where isothermal amplification and double Q-Zip portions containing Q-dots on a single tail are used with universal array capture to score allele imbalance in a tumor sample compared with a normal sample. This figure is similar to FIG. 54, except the Q-dot encryption is used to score allele imbalance in a tumor sample compared with the normal sample. In the initial LDR reaction a given single base difference is interrogated with two allele-specific encrypted oligonucleotide probes in the normal sample in a first tube. The same single base difference is interrogated with two different allele-specific encrypted oligonucleotide probes in the tumor sample in a second tube. Although multiple different single base differences are detected in each tube, for simplicity only SNP1 is shown. The ligation products are combined in a single tube and the LDR product capture, isothermal amplification, and subsequent procedure are the same as described in FIG. 54. Emission spectra at each address are quantified to decode the presence of specific Q-dots, to score allele imbalance in the tumor compared to the normal sample.

The detection results in FIG. 57 show that at SNP1 the normal sample is heterozygous A, G and the tumor sample demonstrates a loss of the A allele and amplification of the G allele. In FIG. 57, the ratio of Q-Zip portions containing Q-dots (Q1,Q5)/(Q4,Q1) at address Zp1 is about zero, indicating loss of the A allele in the tumor. The ratio of Q-Zip portions containing Q-dots (Q8,Q6)/(Q3,Q2) at address Zp1 is about three, indicating an approximately three-fold amplification of the G allele in the tumor.

FIG. 58 is a schematic diagram of an LDR process, in accordance with the present invention, where isothermal amplification and ligated double Q-Zip portions containing Q-dots on a single tail are used with universal array capture to score allele imbalance in a tumor sample compared with a normal sample. FIG. 58 is similar to FIG. 57, but with the addition of ligating adjacent Q-Zip portions containing Q-dots are ligated in the last step.

The detection results in FIG. 58 show that at SNP1 the normal sample is heterozygous A, G and the tumor sample demonstrates a loss of the A allele and amplification of the G allele. In FIG. 58, the ratio of ligated Q-Zip portions containing Q-dots (Q1,Q5)/(Q4,Q1) at address Zp1 is about zero, indicating loss of the A allele in the tumor. The ratio of ligated Q-Zip portions containing Q-dots (Q8,Q6)/(Q3,Q2) at address Zp1 is about three, indicating an approximately three-fold amplification of the G allele in the tumor.

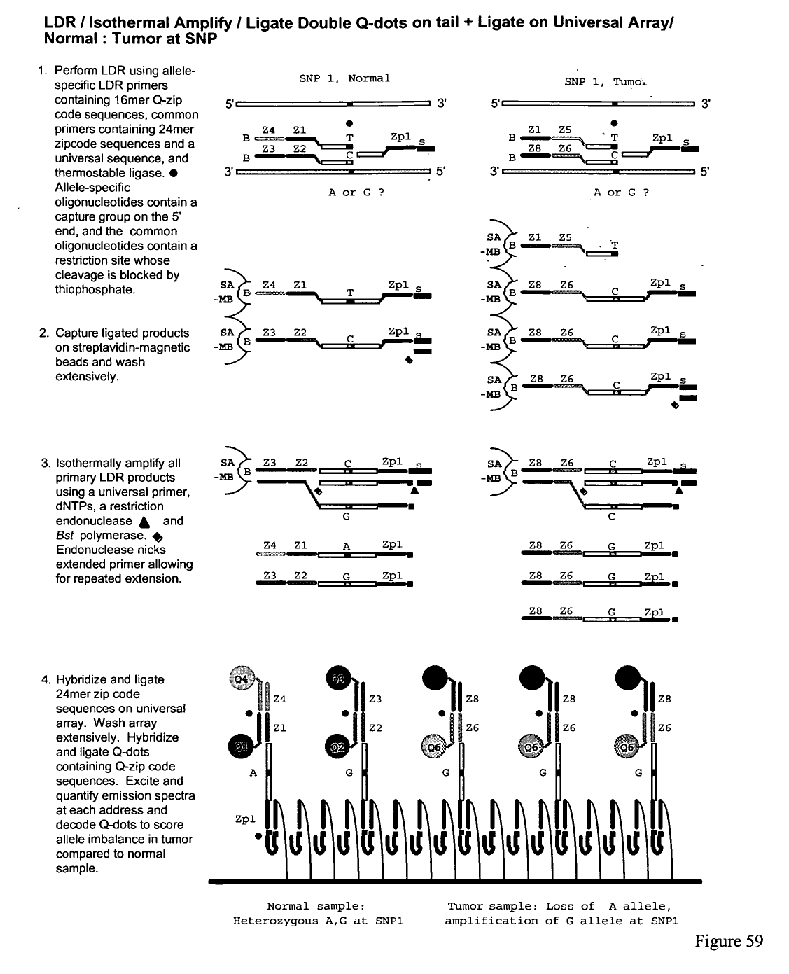

FIG. 59 is a schematic diagram of an LDR process, in accordance with the present invention, where isothermal amplification and ligated double Q-Zip portions containing Q-dots on a single tail are used with ligation to a universal array to score allele imbalance in a tumor sample compared with a normal sample. FIG. 59 is similar to FIG. 58, but with the addition of ligating the captured addressable array-specific portions of the ligation products to their respective capture probes on the array followed by a stringent wash step. Subsequently, adjacent Q-Zip portions containing Q-dots in the last step.

The detection results in FIG. 59 show that at SNP1 the normal sample is heterozygous A, G and the tumor sample demonstrates a loss of the A allele and amplification of the G allele. In FIG. 59, the ratio of ligated Q-Zip portions containing Q-dots (Q1,Q5)/(Q4,Q1) at address Zp1 is about zero, indicating loss of the A allele in the tumor. The ratio of ligated Q-Zip portions containing Q-dots (Q8,Q6)/(Q3,Q2) at address Zp1 is about three, indicating an approximately three-fold amplification of the G allele in the tumor.

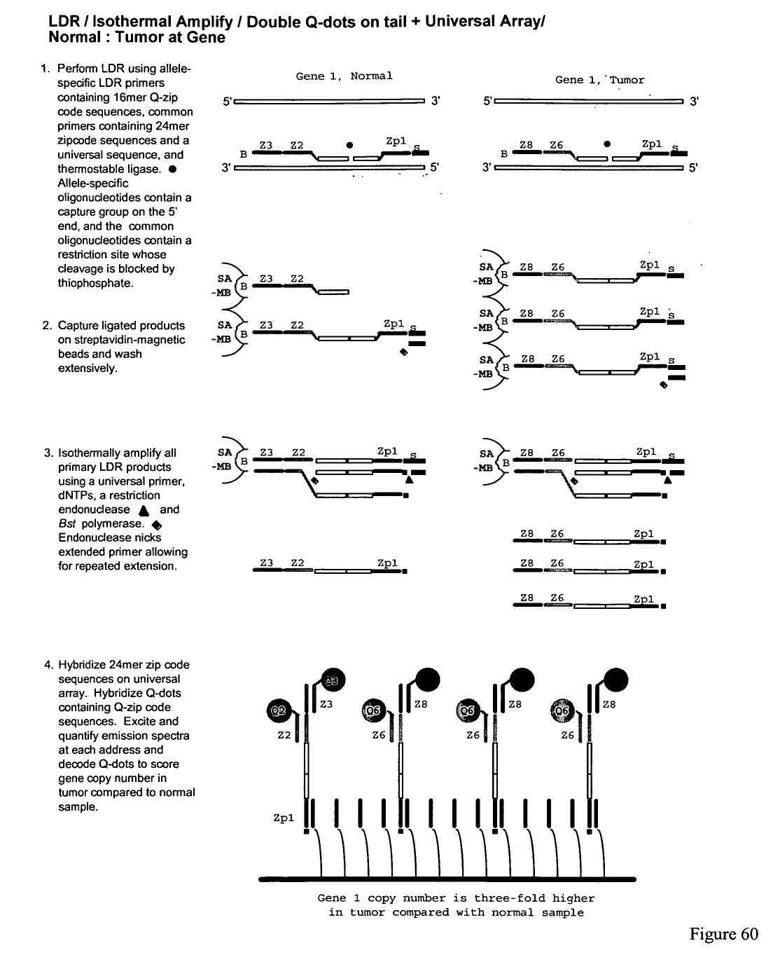

FIG. 60 is a schematic diagram of an LDR process, in accordance with the present invention, where isothermal amplification and double Q-Zip portions containing Q-dots on a single tail are used with universal array capture to score gene copy number in a tumor sample compared with a normal sample. FIG. 60 is similar to FIG. 57, except the Q-dot encryption is used to score gene copy number in a tumor sample compared with the normal sample. In the initial LDR reaction, a given gene is interrogated with an allele-specific encrypted oligonucleotide probes in the normal sample in a first tube. The same gene is interrogated with a different allele-specific encrypted oligonucleotide probes in the tumor sample in a second tube. Although multiple different genes are detected in each tube, for simplicity only one gene is shown. The ligation products are combined in a single tube and the ligation product capture, isothermal amplification, and subsequent procedure are the same as described in FIG. 57. Emission spectra at each address are quantified to decode the presence of specific Q-dots, to score gene copy number in the tumor compared to the normal sample. The detection results in FIG. 60 show an approximately three-fold amplification of gene 1 in the tumor sample (indicated by a ratio of Q-Zip portions containing Q-dots (Q8,Q6)/(Q3,Q2) at address Zp1 of about three).

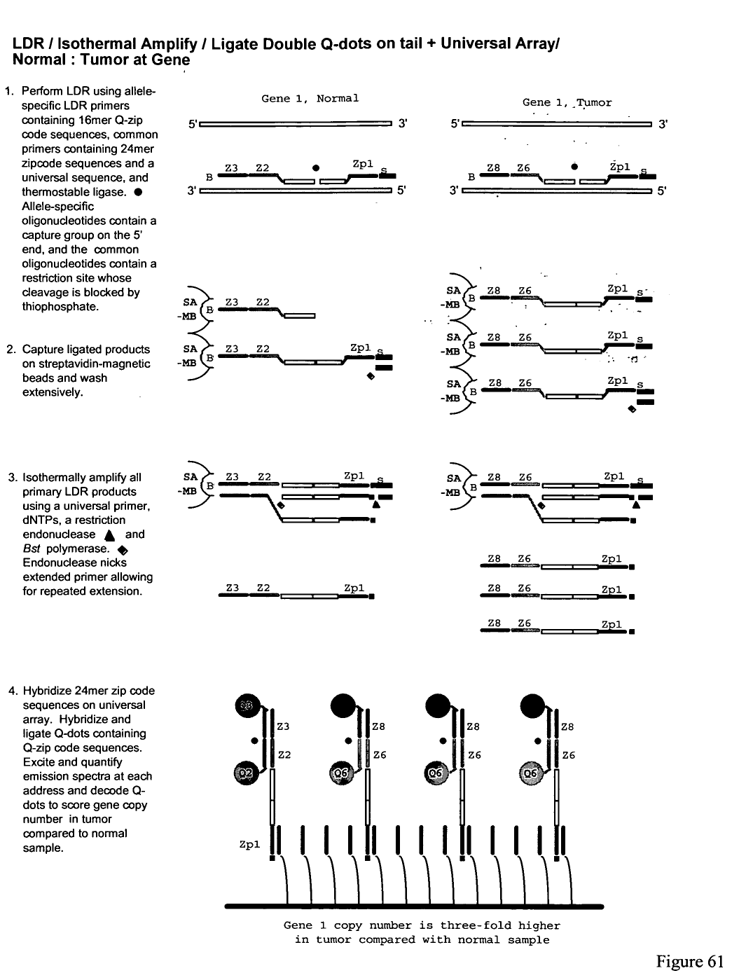

FIG. 61 is a schematic diagram of an LDR process, in accordance with the present invention, where isothermal amplification and ligated double Q-Zip portions containing Q-dots on a single tail are used with universal array capture to score gene copy number in a tumor sample compared with a normal sample. FIG. 61 is similar to FIG. 60, but with the addition of ligating adjacent Q-Zip portions containing Q-dots in the last step. The detection results in FIG. 61 show an approximately three-fold amplification of gene 1 in the tumor sample (indicated by a ratio of ligated Q-Zip portions containing Q-dots (Q8,Q6)/(Q3,Q2) at address Zp1 of about three).

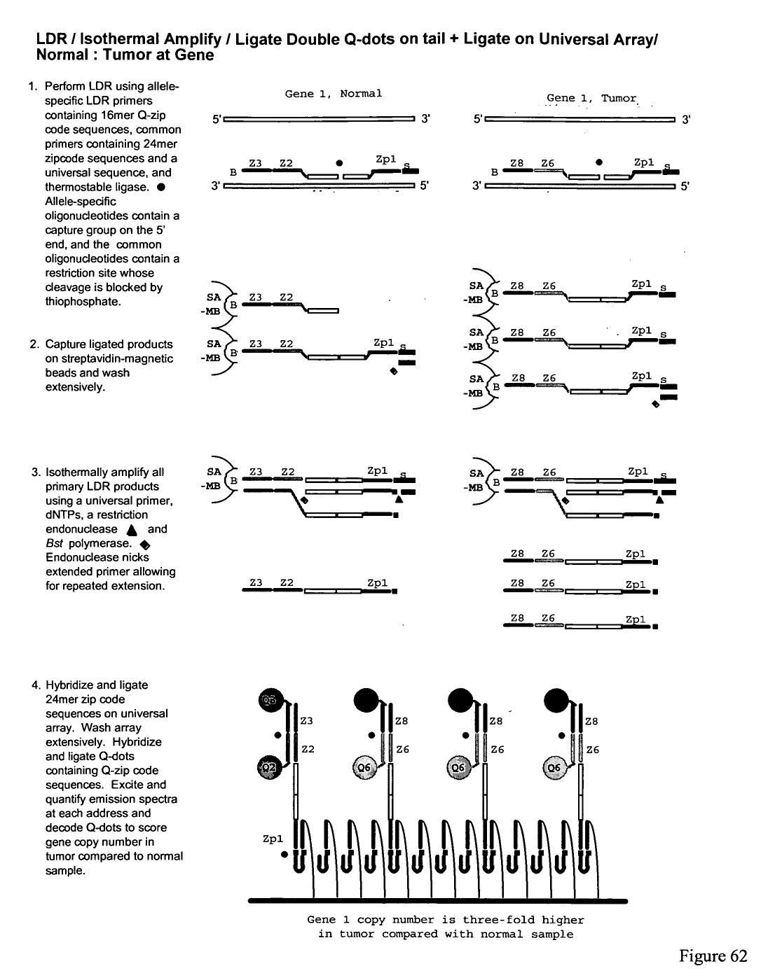

FIG. 62 is a schematic diagram of an LDR process, in accordance with the present invention, where isothermal amplification and ligated double Q-Zip portions containing Q-dots on a single tail are used with ligation to a universal array to score gene copy number in a tumor sample compared with a normal sample. FIG. 62 is similar to FIG. 61, but with the addition of ligating the addressable array-specific portions of the ligation products to their respective capture probes on the array followed by a stringent wash step. Subsequently, adjacent Q-Zip portions containing Q-dots are ligated in the last step. The detection results in FIG. 62 show an approximately three-fold amplification of gene 1 in the tumor sample (indicated by a ratio of ligated Q-Zip portions containing Q-dots (Q8,Q6)/(Q3,Q2) at address Zp1 of about three).

When working with an mRNA target, this aspect of the present invention may further involve, prior to blending, providing a primer complementary to the target mRNA molecule, providing a reverse transcriptase, blending the primer, the reverse transcriptase, and the sample to form a reverse transcription mixture, and subjecting the reverse transcription mixture to a reverse transcription reaction to produce cDNA copies of the target mRNA molecule.

This aspect of the present invention may further involve subjecting the sample to whole genome amplification prior to blending. This may involve providing random primers, providing a polymerase, blending the sample, the random primers, and the polymerase to form a whole genome amplification reaction mixture, and subjecting the whole genome amplification reaction mixture to a polymerase extension reaction under conditions effective to amplify the whole genome. Whole genome amplification is described in Lage et al., “Whole Genome Analysis of Genetic Alterations in Small DNA Samples Using Hyperbranched Strand Displacement Amplification and Array-CGH,” Genome Res. 13:294-307 (2003), which is hereby incorporated by reference in its entirety.

In carrying out the ligase detection reaction, solid support capture, detection probe hybridization, and detection scheme of the present invention, relative amounts of one or more target nucleic acid molecules in the test sample, differing by one or more single-base changes, insertions, deletions, or translocations, or one or more target mRNA molecules differing by one or more splice site variations, may be quantified by comparison with a reference sample having reference target nucleic acid molecules or reference target mRNA molecules.

This involves providing one or more secondary oligonucleotide probe sets, which differ from the primary oligonucleotide probe sets. Each of the secondary oligonucleotide probes sets are characterized by (a) a first oligonucleotide probe, having one or more detection oligonucleotide probe-specific portions or their complements and a reference target-specific portion, and (b) a second oligonucleotide probe, having a reference target-specific portion and an addressable array-specific portion or its complement. The oligonucleotide probes in a particular secondary oligonucleotide probe set are suitable for ligation together when hybridized to one another on a corresponding reference target nucleic acid molecule or reference target mRNA molecules, but have a mismatch which interferes with this ligation when hybridized to any other nucleic acid or mRNA molecule present in the reference sample. One or both oligonucleotide probes in the secondary oligonucleotide probe set contain one or more detection oligonucleotide probe-specific portions or their complements so that each secondary oligonucleotide probe set contains a unique set of one or more detection oligonucleotide probe-specific portions or their complements.

The reference sample, the one or more secondary oligonucleotide probe sets, and the ligase are blended to form a secondary ligase detection reaction mixture. The secondary ligase detection reaction mixture is then subjected to one or more ligase detection reaction cycles comprising a denaturation treatment and a hybridization treatment. In the denaturation treatment, any hybridized oligonucleotides are separated from reference target nucleic acid molecules or reference target mRNA molecules. During the hybridization treatment, the oligonucleotide probe sets hybridize in a base-specific manner to their respective reference target nucleic acid molecule or reference target mRNA molecules, if present in the reference sample, and ligate to one another to form a secondary ligation product containing (a) the one or more detection oligonucleotide probe-specific portions or their complements, (b) the reference sample-specific portions and (c) the addressable array-specific portion or its complement. The secondary ligation products for each secondary oligonucleotide probe set are distinguishable from other nucleic acid molecules or mRNA molecules in the secondary ligase detection reaction mixture. The secondary oligonucleotide probe sets may hybridize to nucleic acid molecules or mRNA molecules in the reference sample other than their respective reference target nucleic acid molecules or reference target mRNA molecules but do not ligate together due to the presence of one or more mismatches and individually separate during the denaturation treatment.

The primary and secondary ligase detection reaction mixtures are blended after subjecting them to one or more ligase detection reaction cycles and before capturing, whereby the blended first and second ligase detection reaction mixtures are subjected to capturing, contacting, and detecting. The relative amounts of the reporter labels on the primary and secondary ligation products are compared, to provide a quantitative measure of the relative level of the one or more target nucleic acid molecules or target mRNA molecules in the test sample compared with that of the reference target nucleic acid molecules or reference target mRNA molecules in the reference sample as a result of each different labeled ligation product having a unique encryption code. This aspect of the present invention is useful for quantifying, without limitation, gene copy number, mRNA copy number, or mRNA splice variant copy number.

Solid Support Capture, Ligase Detection Reaction, Detection Probe Hybridization, and Detection Scheme

Another embodiment of the present invention relates to a method for identifying one or more target nucleic acid molecules, differing by one or more single-base changes, insertions, deletions, or translocations, in a plurality of nucleic acid molecules or identifying one or more target mRNA molecules differing by one or more splice site variations in a plurality of mRNA molecules. This includes solid support capture, ligase detection reaction, detection probe hybridization, and detection.

The solid support capture phase, as described above, involves providing a sample potentially containing one or more target nucleic acid molecules differing by one or more single-base changes, insertions, deletions, or translocations or one or more target mRNA molecules differing by one or more splice site variations. The target nucleic acid molecules or target mRNA molecules in the sample are captured on one or a plurality of solid supports, so that they may be individually distinguished.

In the ligase detection reaction phase, as described above, one or more primary oligonucleotide probe sets are provided, each set characterized by (a) a first oligonucleotide probe, having a target-specific portion and a 5′ upstream portion complementary to one or more detection oligonucleotide probes and (b) a second oligonucleotide probe, having a target-specific portion and a 3′ downstream portion complementary to one or more detection oligonucleotide probes. The oligonucleotide probes in a particular set are suitable for ligation together when hybridized to one another on a corresponding target nucleic molecule, but have a mismatch which interferes with this ligation when hybridized to any other nucleic molecule present in the sample. Each probe set contains a unique set of 5′ upstream and 3′ downstream portions. The sample, the one or more primary oligonucleotide probe sets, and the ligase are blended to form a primary ligase detection reaction mixture. The primary ligase detection reaction mixture is subjected to one or more ligase detection reaction cycles. These cycles include a denaturation treatment and hybridization treatment. In the denaturation treatment, any hybridized oligonucleotides are separated from the target nucleic acid molecule or target mRNA molecule. For the hybridization treatment, the oligonucleotide probe sets hybridize in a base-specific manner to their respective target nucleic acid molecules or target mRNA molecule, if present in the sample, and ligate to one another to form a primary ligation product. The primary ligation product contains (a) the 5′ upstream portion, (b) the target-specific portions, and (c) the 3′ downstream portion with the primary ligation product remaining bound to the captured target nucleic acid molecule or target mRNA molecule. The primary ligation product for each primary oligonucleotide probe set is distinguishable from other nucleic acids in the primary ligase detection reaction mixture by virtue of its containing a unique set of 5′ upstream portion and 3′ downstream portion. The oligonucleotide probe sets may hybridize to nucleic acid molecules in the sample other than their respective target nucleic acid molecule or target mRNA sequences but do not ligate together due to the presence of one or more mismatches and individually separate during the denaturation treatment.

In the detection probe hybridization phase, as described above, detection oligonucleotide probes which bind to the 5′ upstream and 3′ downstream portions of the ligation products are provided. Each detection oligonucleotide probe has a reporter label, so that each labeled primary ligation product has a unique detectable encryption code. The primary ligation products and the detection oligonucleotide probes are contacted under conditions effective to permit hybridization of the detection oligonucleotide probes to the primary ligation products so that a labeled captured primary ligation product is formed.

In the detection phase, as described above, the reporter labels on the primary ligation products are detected. As a result, the presence of one or more target nucleic acid molecules differing by one or more single base changes, insertions, deletions, or translocations or one or more target mRNA molecules differing by one or more splice site variations are discriminated from one another during the ligase detection reaction. The discriminated target nucleic acid molecules or the target mRNA molecules are detected due to each different reporter label having a unique encryption code with a different pattern of detectable emission spectra.

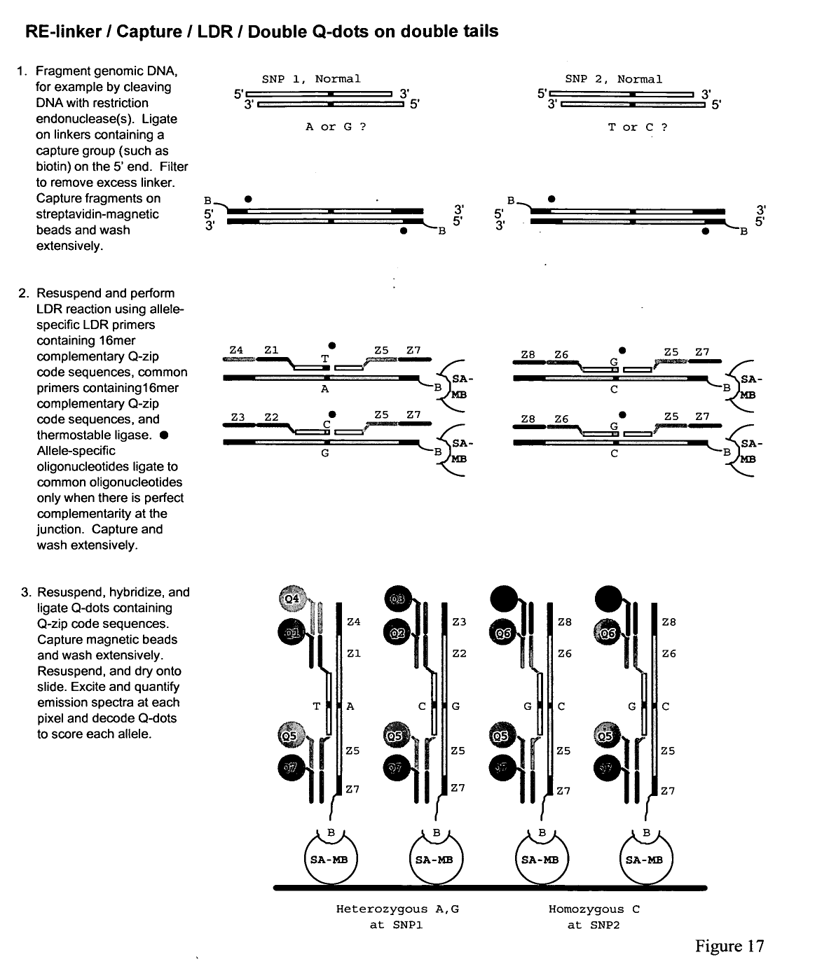

Examples of the solid support capture, ligase detection reaction, detection probe hybridization, and detection scheme are shown in FIGS. 17-36.

FIG. 17 is a schematic diagram of an LDR process, in accordance with the present invention, where double Q-Zip portions containing Q-dots on double tails are used with microbead capture to distinguish single base differences in nucleic acid molecules. Genomic DNA fragments are generated by cleaving DNA with restriction endonuclease(s). Linkers containing a capture group (such as biotin) are ligated onto the 5′ end. Excess linker is removed using filtration or size-exclusion columns. Genomic fragments are captured on streptavidin-magnetic beads and washed extensively. Magnetic beads containing the genomic fragments are resuspended, and LDR is performed directly on the nucleic acid attached to beads using allele-specific oligonucleotide probes containing 16mer complementary Q-Zip portions containing Q-dots, common oligonucleotide probes containing 16mer complementary Q-Zip portions containing Q-dots, and thermostable ligase. Allele-specific oligonucleotide probes (Z4,Z1), (Z3,Z2), (Z1,Z5), and (Z8,Z6) discriminate A, G, T, and C, respectively, on the target 3′->5′ nucleotide sequence. Allele-specific oligonucleotide probes ligate to common oligonucleotide probes only when there is perfect complementarity at the junction. Subsequently, magnetic beads are captured, washed extensively, resuspended, and hybridized with Q-Zip portions containing Q-dots. After capture and extensive washing, the magnetic beads are resuspended and dried onto a slide. Emission spectra at each pixel are quantified to decode the presence of specific Q-dots, to score each allele.

The detection results in FIG. 17 show that the sample is heterozygous A, G at SNP1 and homozygous C at SNP2. The A allele is indicated by the Q-Zip portions containing Q-dots (Q4,Q1,Q5,Q7) hybridizing to the ligation product having addressable array-specific portion (Z4, Z1). The G allele is indicated by the Q-Zip portions containing Q-dots (Q3,Q2,Q5,Q7) hybridizing to the ligation product having addressable array-specific portions (Z3,Z2). The C allele is indicated by the Q-Zip portions containing Q-dots (Q8,Q6,Q5,Q7) hybridizing to the ligation product having addressable array-specific portions (Z8,Z6).

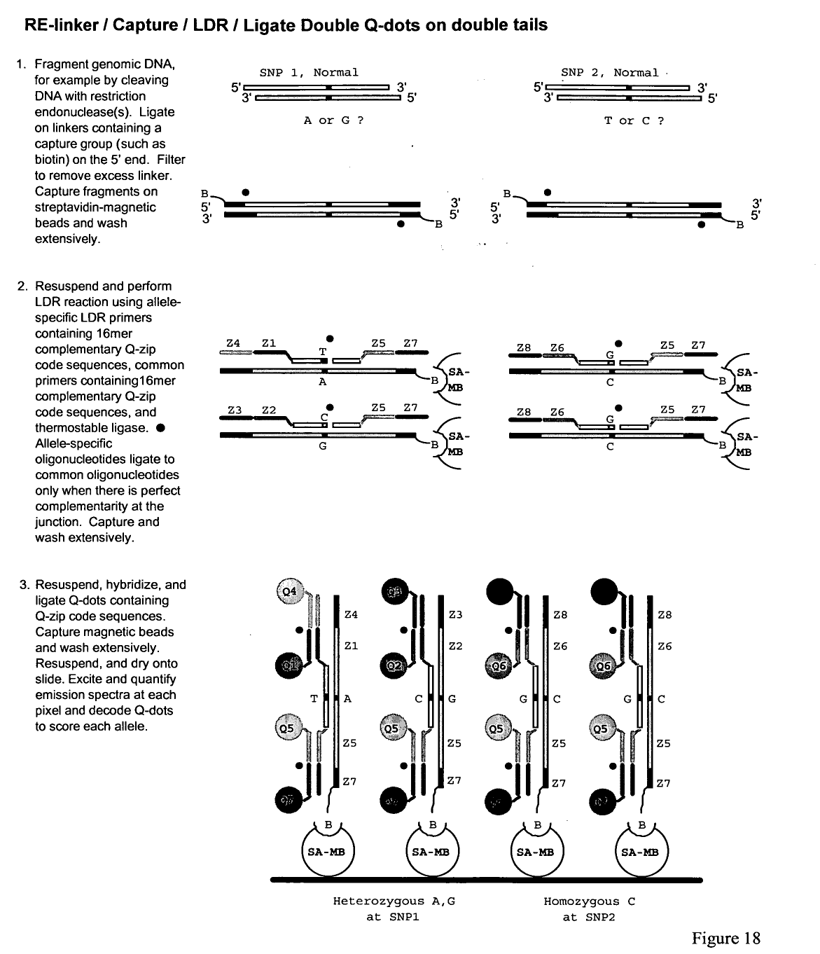

FIG. 18 is a schematic diagram of an LDR process, in accordance with the present invention, where ligated double Q-Zip portions containing Q-dots on double tails are used with microbead capture to distinguish single base differences in nucleic acid molecules. FIG. 18 is similar to FIG. 17, but with the addition of ligating adjacent Q-Zip portions containing Q-dots in the last step.

The detection results in FIG. 18 show that the sample is heterozygous A, G at SNP1 and homozygous C at SNP2. The A allele is indicated by the ligated Q-Zip portions containing Q-dots (Q4,Q1,Q5,Q7) hybridizing to the ligation product having portions containing Q-dots (Z4,Z1). The G allele is indicated by the ligated Q-Zip portions containing Q-dots (Q3,Q2,Q5,Q7) hybridizing to the ligation product having Q-Zip portions containing Q-dots (Z3,Z2). The C allele is indicated by the ligated Q-Zip portions containing Q-dots (Q8,Q6,Q5,Q7) hybridizing to the ligation product having portions containing Q-dots (Z8,Z6).

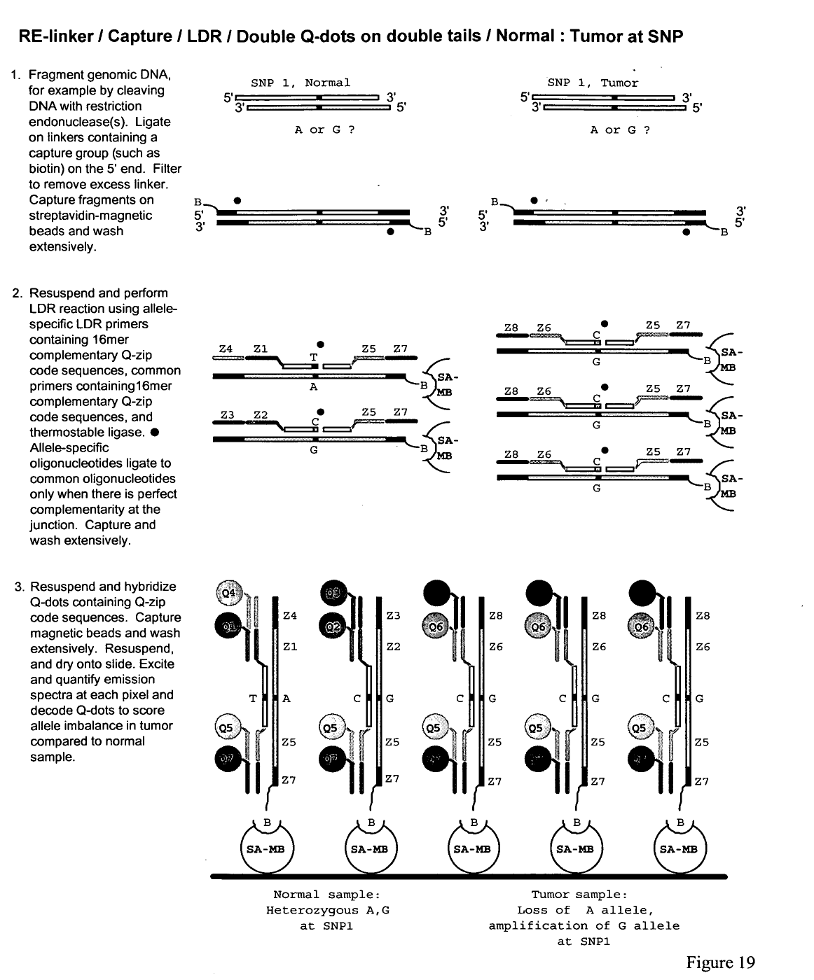

FIG. 19 is a schematic diagram of an LDR process, in accordance with the present invention, where double Q-Zip portions containing Q-dots on double tails are used with microbead capture to score allele imbalance in a tumor sample compared with a normal sample. This figure is similar to FIG. 17, except the Q-dot encryption is used to score allele imbalance in a tumor sample compared with the normal sample. Genomic fragments are captured on streptavidin-magnetic beads and washed extensively as described above. Tumor and normal samples are kept separate. In the initial LDR reaction a given single base difference is interrogated with two allele-specific encrypted oligonucleotide probes in the normal sample in a first tube. The same single base difference is interrogated with two different allele-specific encrypted oligonucleotide probes in the tumor sample in a second tube. Although multiple different single base differences are detected in each tube, for simplicity only SNP1 is shown. After the ligation detection reaction, the ligation products on magnetic beads are combined in a single tube and washed extensively. The beads are resuspended and dried onto a microscope slide. Emission spectra at each pixel are quantified to decode the presence of specific Q-dots, to score allele imbalance in the tumor compared to the normal sample.

The detection results in FIG. 19 show that at SNP1 the normal sample is heterozygous A, G and the tumor sample demonstrates a loss of the A allele and amplification of the G allele. In FIG. 19, the ratio of hybridized Q-Zip portions containing Q-dots (Q1,Q5,Q5,Q7)/(Q4,Q1,Q5,Q7) is about zero, indicating loss of the A allele in the tumor. The ratio of hybridized Q-Zip portions containing Q-dots (Q8,Q6,Q5,Q7)/(Q3,Q2,Q5,Q7) is about three, indicating an approximately three-fold amplification of the G allele in the tumor.

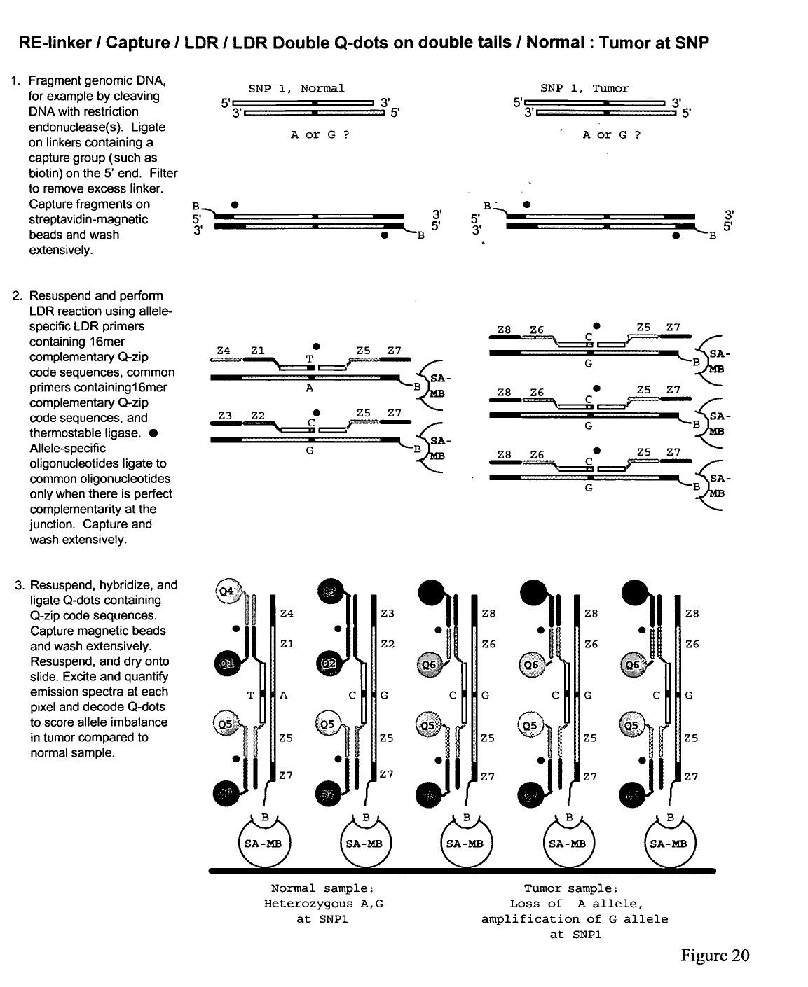

FIG. 20 is a schematic diagram of an LDR process, in accordance with the present invention, where ligated double Q-Zip portions containing Q-dots on double tails are used with microbead capture to score allele imbalance in a tumor sample compared with a normal sample. FIG. 20 is similar to FIG. 19, but with the addition of ligating adjacent Q-Zip portions containing Q-dots in the last step.

The detection results in FIG. 20 show that at SNP1 the normal sample is heterozygous A, G and the tumor sample demonstrates a loss of the A allele and amplification of the G allele. In FIG. 20, the ratio of hybridized ligated Q-Zip portions containing Q-dots (Q1,Q5,Q5,Q7)/(Q4,Q1,Q5,Q7) is about zero, indicating loss of the A allele in the tumor. The ratio of hybridized ligated Q-Zip portions containing Q-dots (Q8,Q6,Q5,Q7)/(Q3,Q2,Q5,Q7) is about three, indicating an approximately three-fold amplification of the G allele in the tumor.

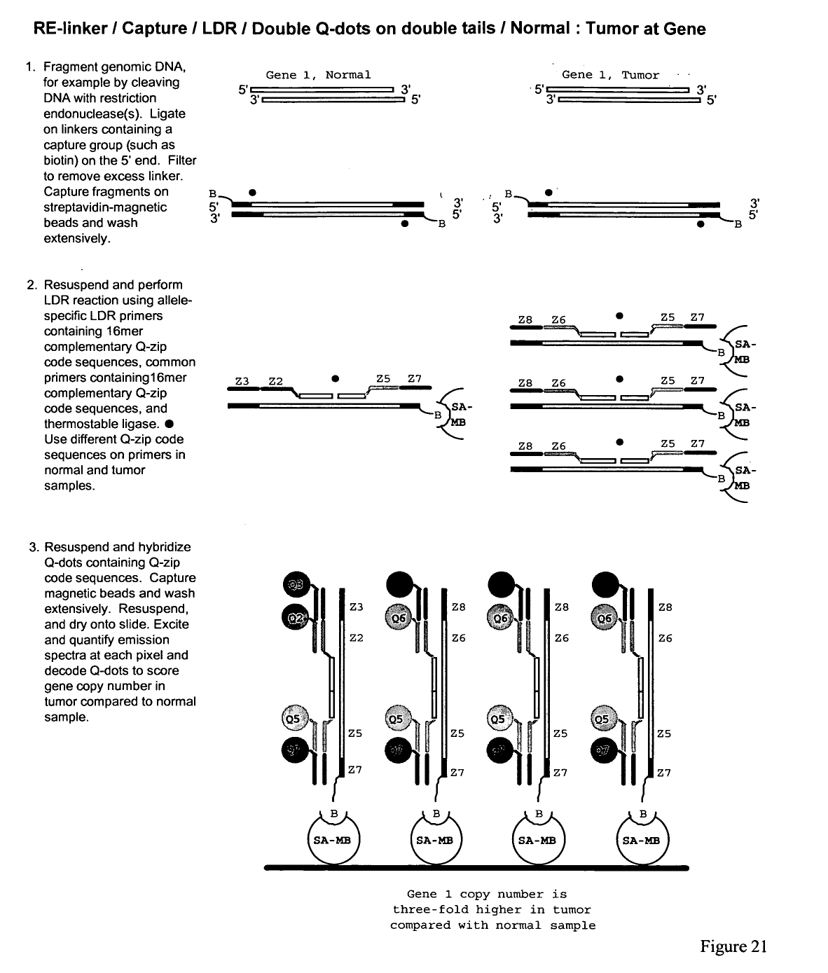

FIG. 21 is a schematic diagram of an LDR process, in accordance with the present invention, where double Q-Zip portions containing Q-dots on double tails are used with microbead capture to score gene copy number in a tumor sample compared with a normal sample. This figure is similar to FIG. 17, except the Q-dot encryption is used to score allele imbalance in a tumor sample compared with the normal sample. Genomic fragments are captured on streptavidin-magnetic beads and washed extensively as described above. Tumor and normal samples are kept separate. In the initial LDR reaction, a given gene is interrogated with a gene-specific encrypted oligonucleotide probe in the normal sample in a first tube. The same gene is interrogated with a different gene-specific encrypted oligonucleotide probe in the tumor sample in a second tube. Although multiple different genes are detected in each tube, for simplicity only gene1 is shown. After ligase detection reaction, the ligation products on magnetic beads are combined in a single tube and washed extensively. The beads are resuspended and dried onto a slide. Emission spectra at each pixel are quantified to decode the presence of specific Q-dots, to score gene copy number in the tumor compared to the normal sample. The detection results in FIG. 21 show an approximately three-fold amplification of gene 1 in the tumor sample (indicated by a ratio of hybridized Q-Zip portions containing Q-dots (Q8,Q6,Q5,Q7)/(Q3,Q2,Q5,Q7) of about three).

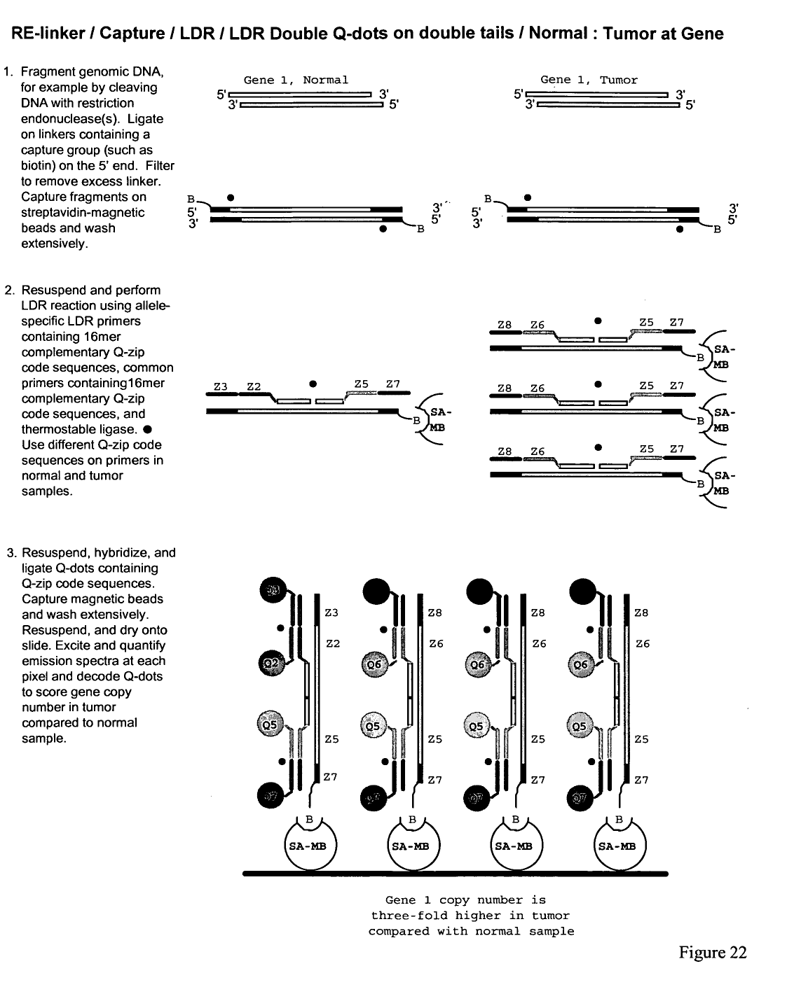

FIG. 22 is a schematic diagram of an LDR process, in accordance with the present invention, where ligated double Q-Zip portions containing Q-dots on double tails are used with microbead capture to score gene copy number in a tumor sample compared with a normal sample. FIG. 22 is similar to FIG. 21, but with the addition of ligating adjacent Q-Zip portions containing Q-dots in the last step. The detection results in FIG. 22 show an approximately three-fold amplification of gene 1 in the tumor sample (indicated by a ratio of hybridized ligated Q-Zip portions containing Q-dots (Q8,Q6,Q5,Q7)/(Q3,Q2,Q5,Q7) of about three).

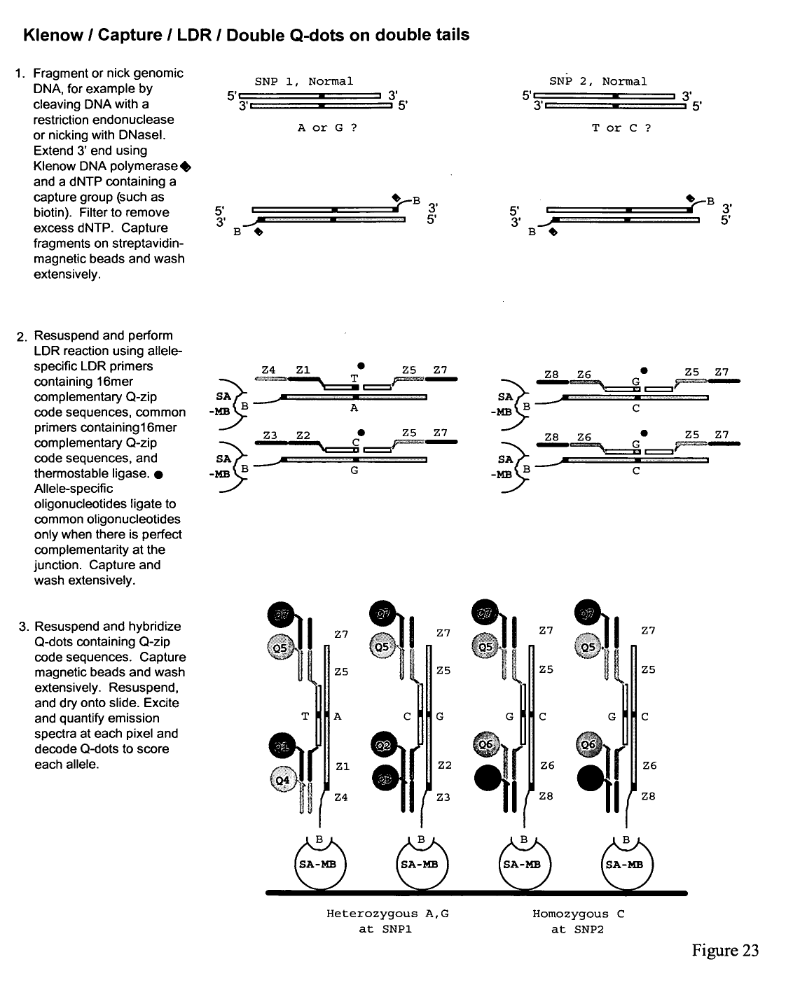

FIG. 23 is a schematic diagram of an LDR process, in accordance with the present invention, where double Q-Zip portions containing Q-dots on double tails are used with microbead capture to distinguish single base differences in nucleic acids. Genomic DNA fragments are generated by cleaving DNA with restriction endonuclease(s), or nicking with DNase. 3′ ends are extended with a polymerase such as Klenow using a dNTP containing a capture group (such as biotin). Excess dNTP is removed using filtration or size-exclusion columns. Genomic fragments are captured on streptavidin-magnetic beads and washed extensively. Magnetic beads containing the genomic fragments are resuspended, and LDR is performed directly on DNA attached to beads using allele-specific oligonucleotide probes containing 16mer complementary Q-Zip portions, common oligonucleotide probes containing 16mer complementary Q-Zip portions, and thermostable ligase. Allele-specific oligonucleotide probes (Z4,Z1), (Z3,Z2), (Z1,Z5), and (Z8,Z6) discriminate A, G, T, and C, respectively, on the target 3′→5′ nucleotide sequence. Allele-specific oligonucleotide probes ligate to common oligonucleotide probes only when there is perfect complementarity at the junction. Subsequently, magnetic beads are captured, washed extensively, resuspended, and hybridized with Q-Zip portions containing Q-dots. After capture and extensive washing, the magnetic beads are resuspended and dried onto a microscope slide. Emission spectra at each pixel are quantified to decode the presence of specific Q-dots, to score each allele.

The detection results in FIG. 23 show that the sample is heterozygous A, G at SNP1 and homozygous C at SNP2. The A allele is indicated by the Q-Zip portions containing Q-dots (Q4,Q1,Q5,Q7) hybridizing to the ligation product having addressable array-specific portions (Z4,Z1). The G allele is indicated by the Q-Zip portions containing Q-dots (Q3,Q2,Q5,Q7) hybridizing to the ligation product having addressable array-specific portions (Z3,Z2). The C allele is indicated by the Q-Zip portions containing Q-dots (Q8,Q6,Q5,Q7) hybridizing to the ligation product having addressable array-specific portions (Z8,Z6).

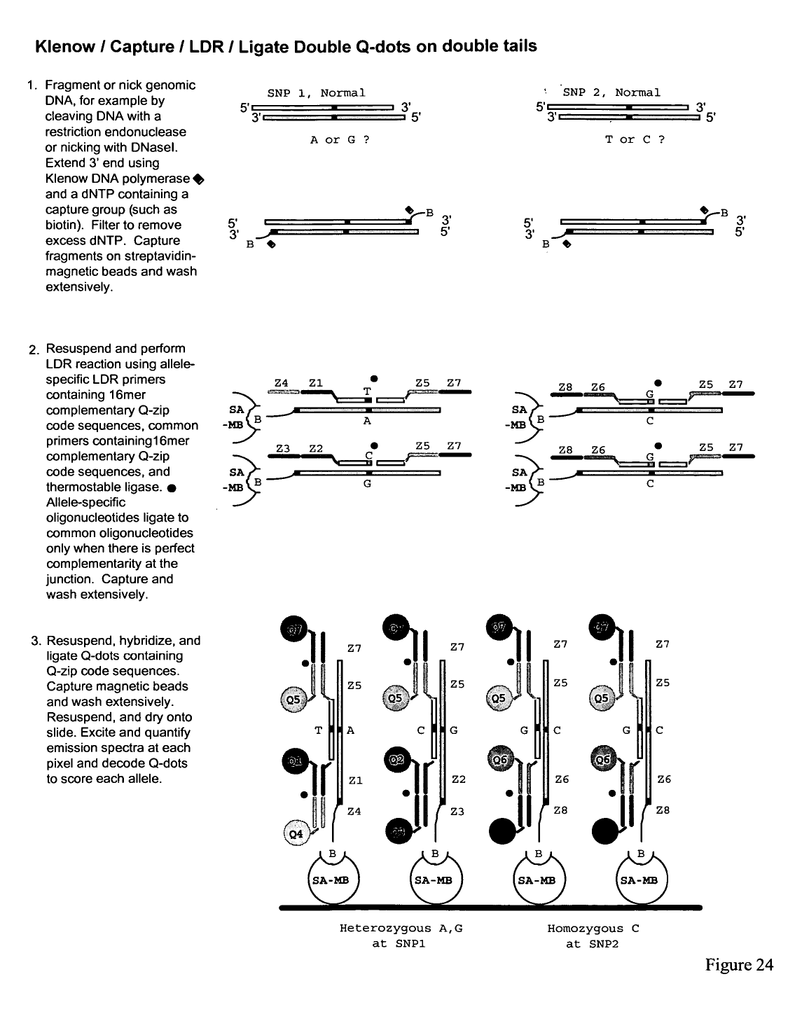

FIG. 24 is a schematic diagram of an LDR process, in accordance with the present invention, where ligated double Q-Zip portions containing Q-dots on double tails are used with microbead capture to distinguish single base differences in nucleic acid molecules. FIG. 24 is similar to FIG. 23, but with the addition of ligating adjacent Q-Zip portions containing Q-dots in the last step.

The detection results in FIG. 24 show that the sample is heterozygous A, G at SNP1 and homozygous C at SNP2. The A allele is indicated by the ligated Q-Zip portions containing Q-dots (Q4,Q1,Q5,Q7) hybridizing to the ligation product having addressable array-specific portions (Z4,Z1); the G allele is indicated by the ligated Q-Zip portions containing Q-dots (Q3,Q2,Q5,Q7) hybridizing to the ligation product having addressable array-specific portions (Z3,Z2). The C allele is indicated by the ligated Q-Zip portions containing Q-dots (Q8,Q6,Q5,Q7) hybridizing to the ligation product having addressable array-specific portions (Z8,Z6).

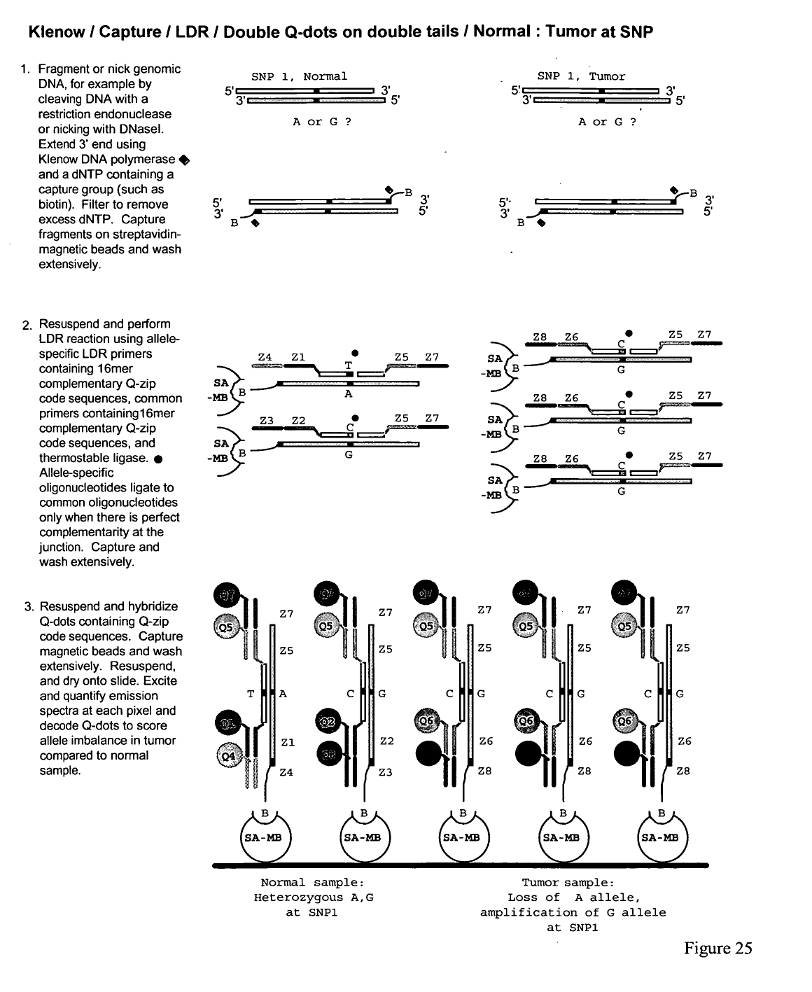

FIG. 25 is a schematic diagram of an LDR process, in accordance with the present invention, where double Q-Zip portions containing Q-dots on double tails are used with microbead capture to score allele imbalance in a tumor sample compared with a normal sample. This figure is similar to FIG. 23, except the Q-dot encryption is used to score allele imbalance in a tumor sample compared with the normal sample. Genomic fragments are captured on streptavidin-magnetic beads and washed extensively as described above. Tumor and normal samples are kept separate. In the initial LDR reaction, a given single base difference is interrogated with two allele-specific encrypted oligonucleotide probes in the normal sample in a first tube. The same single base difference is interrogated with two different allele-specific encrypted oligonucleotide probes in the tumor sample in a second tube. Although multiple different single base differences are detected in each tube, for simplicity only SNP1 is shown. After the ligase detection reaction, ligation products on magnetic beads are combined in a single tube and washed extensively. The beads are resuspended and dried onto a microscope slide. Emission spectra at each pixel are quantified to decode the presence of specific Q-dots, to score allele imbalance in the tumor compared to the normal sample.

The detection results in FIG. 25 show that at SNP1 the normal sample is heterozygous A, G and the tumor sample demonstrates a loss of the A allele and amplification of the G allele. In FIG. 25, the ratio of hybridized Q-Zip portions containing Q-dots (Q1,Q5,Q5,Q7)/(Q4,Q1,Q5,Q7) is about zero, indicating loss of the A allele in the tumor. The ratio of hybridized Q-Zip portions containing Q-dots (Q8,Q6,Q5,Q7)/(Q3,Q2,Q5,Q7) is about three, indicating an approximately three-fold amplification of the G allele in the tumor.

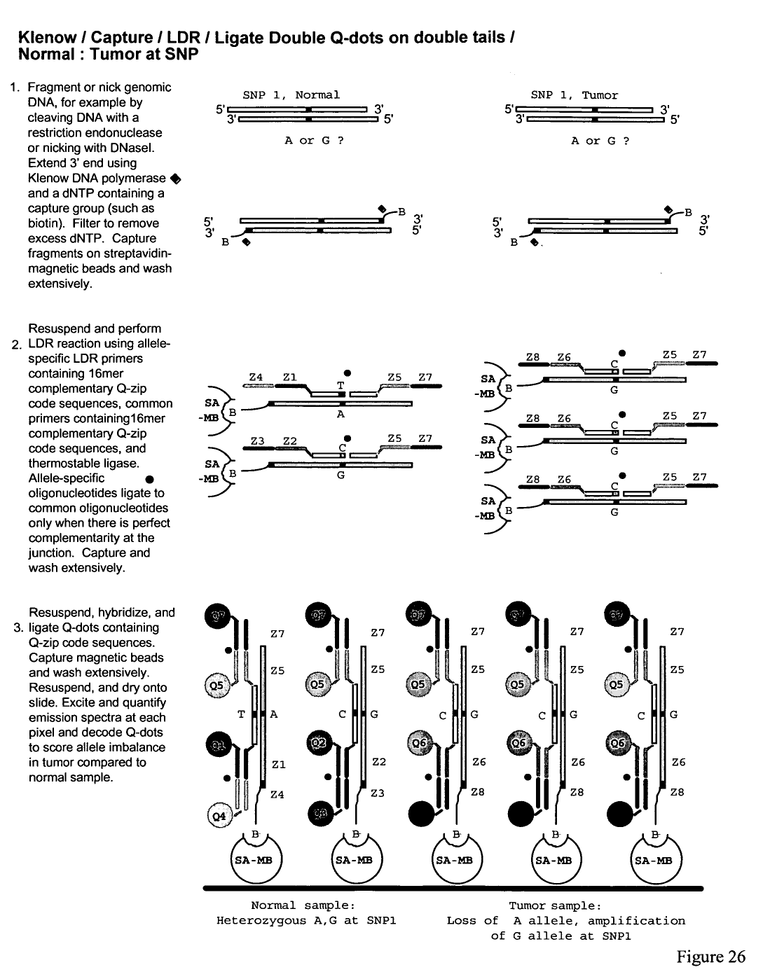

FIG. 26 is a schematic diagram of an LDR process, in accordance with the present invention, where ligated double Q-Zip portions containing Q-dots on double tails are used with microbead capture to score allele imbalance in a tumor sample compared with a normal sample. FIG. 26 is similar to FIG. 25, but with the addition of ligating adjacent Q-Zip portions containing Q-dots in the last step.

The detection results in FIG. 26 show that at SNP1 the normal sample is heterozygous A, G and the tumor sample demonstrates a loss of the A allele and amplification of the G allele. In FIG. 26, the ratio of hybridized ligated Q-Zip portions containing Q-dots (Q1,Q5,Q5,Q7)/(Q4,Q1,Q5,Q7) is about zero, indicating loss of the A allele in the tumor. The ratio of hybridized ligated Q-Zip portions containing Q-dots (Q8,Q6,Q5,Q7)/(Q3,Q2,Q5,Q7) is about three, indicating an approximately three-fold amplification of the G allele in the tumor.

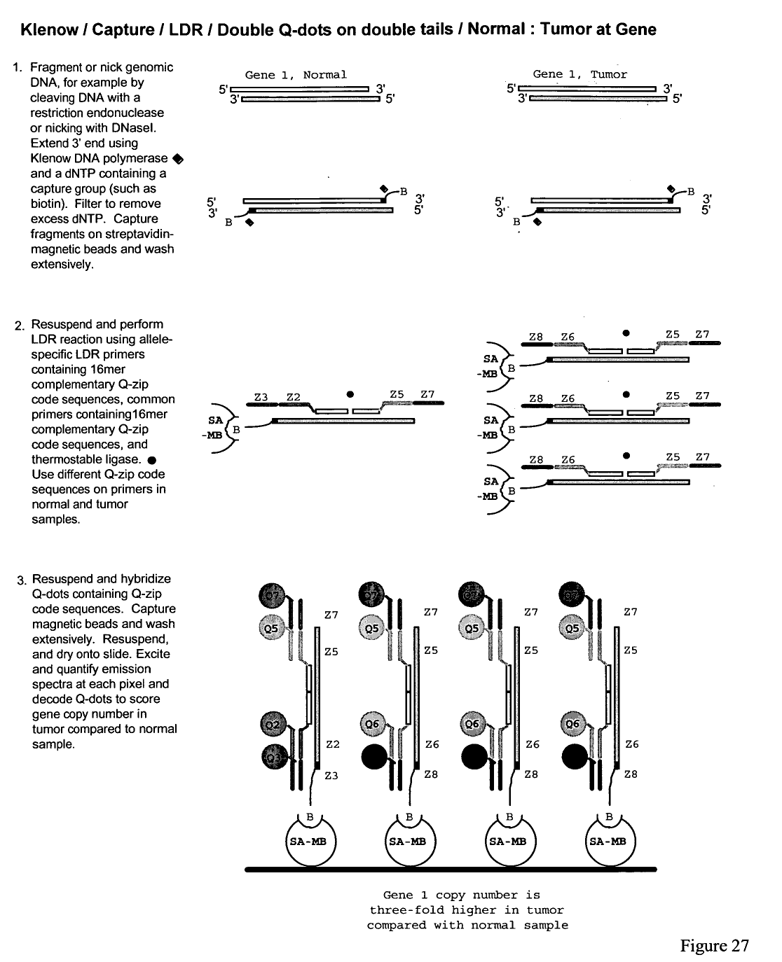

FIG. 27 is a schematic diagram of an LDR process, in accordance with the present invention, where double Q-Zip portions containing Q-dots on double tails are used with microbead capture to score gene copy number in a tumor sample compared with a normal sample. This figure is similar to FIG. 23, except the Q-dot encryption is used to score gene copy number in a tumor sample compared with the normal sample. Genomic fragments are captured on streptavidin-magnetic beads and washed extensively as described above. Tumor and normal samples are kept separate. In the initial LDR reaction, a given gene is interrogated with a gene-specific encrypted oligonucleotide probe in the normal sample in a first tube. The same gene is interrogated with a different gene-specific encrypted oligonucleotide probe in the tumor sample in a second tube. Although multiple different genes are detected in each tube, for simplicity only gene 1 is shown. After the ligase detection reaction, the ligation products on magnetic beads are combined in a single tube and washed extensively. The beads are resuspended and dried onto a microscope slide. Emission spectra at each pixel are quantified to decode the presence of specific Q-dots, to score gene copy number in the tumor compared to the normal sample. The detection results in FIG. 27 show an approximately three-fold amplification of gene 1 in the tumor sample (indicated by a ratio of hybridized Q-Zip portions containing Q-dots (Q8,Q6,Q5,Q7)/(Q3,Q2,Q5,Q7) of about three).

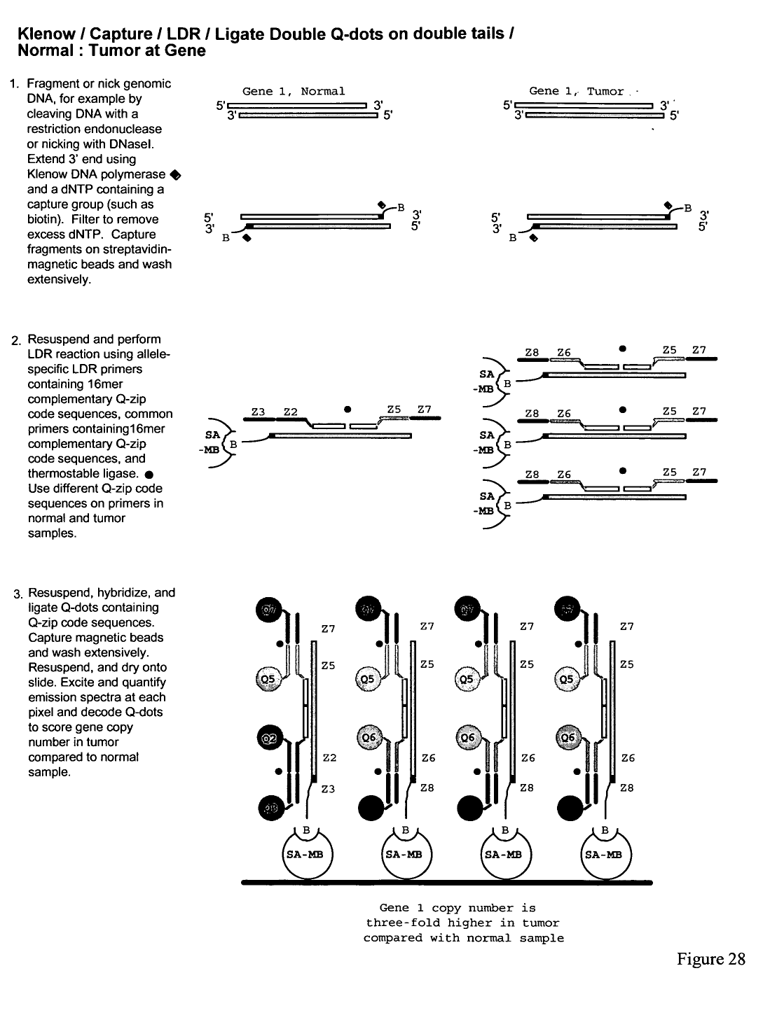

FIG. 28 is a schematic diagram of an LDR process, in accordance with the present invention, where ligated double Q-Zip portions containing Q-dots on double tails are used with microbead capture to score gene copy number in a tumor sample compared with a normal sample. FIG. 28 is similar to FIG. 27, but with the addition of ligating adjacent Q-Zip portions containing Q-dots in the last step. The detection results in FIG. 28 show an approximately three-fold amplification of gene 1 in the tumor sample (indicated by a ratio of hybridized ligated Q-Zip portions containing Q-dots (Q8,Q6,Q5,Q7)/(Q3,Q2,Q5,Q7) of about three).

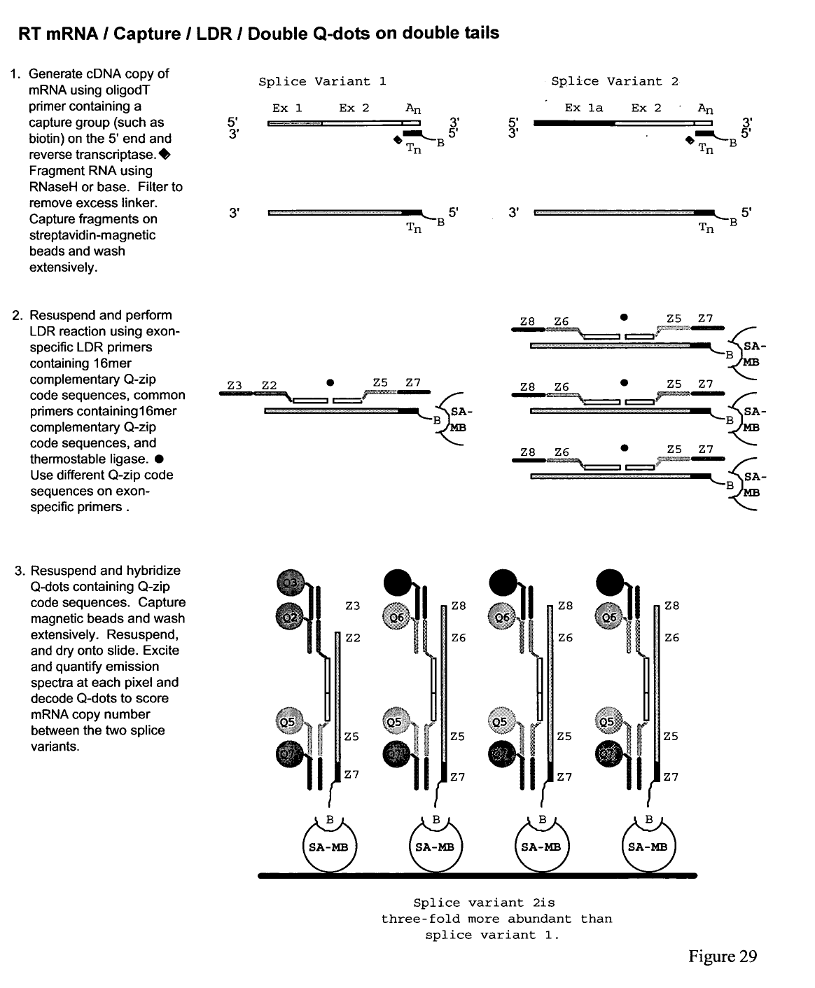

FIG. 29 is a schematic diagram of an LDR process, in accordance with the present invention, where double Q-Zip portions containing Q-dots on double tails are used with microbead capture to distinguish and quantify splice variants on mRNA. A cDNA copy of mRNA is generated using reverse transcriptase on an oligodT primer containing a capture group (such as biotin) on the 5′ end. RNA is fragmented using RNaseH or base. Excess linkers are removed using filtration or size-exclusion columns. cDNAs are captured on streptavidin-magnetic beads and washed extensively. Magnetic beads containing the cDNAs are resuspended, and LDR performed directly on cDNA attached to beads using exon-specific oligonucleotide probes containing 16mer complementary Q-Zip portions containing Q-dots, common oligonucleotide probes containing 16mer complementary Q-Zip portions containing Q-dots, and thermostable ligase. Different Q-Zip probes are used on exon-specific oligonucleotide probes. Subsequently, magnetic beads are captured, washed extensively, resuspended, and hybridized with Q-Zip portions containing Q-dots. After capture and extensive washing, the magnetic beads are resuspended and dried onto a microscope slide. Emission spectra at each pixel are quantified to decode the presence of specific Q-dots, to score mRNA copy number between the two splice variants. The detection results in FIG. 29 show an approximately three-fold greater amount of splice variant containing Ex1a over the splice variant containing Ex1 (indicated by a ratio of hybridized Q-Zip portions containing Q-dots (Q8,Q6,Q5,Q7)/(Q3,Q2,Q5,Q7) of about three).

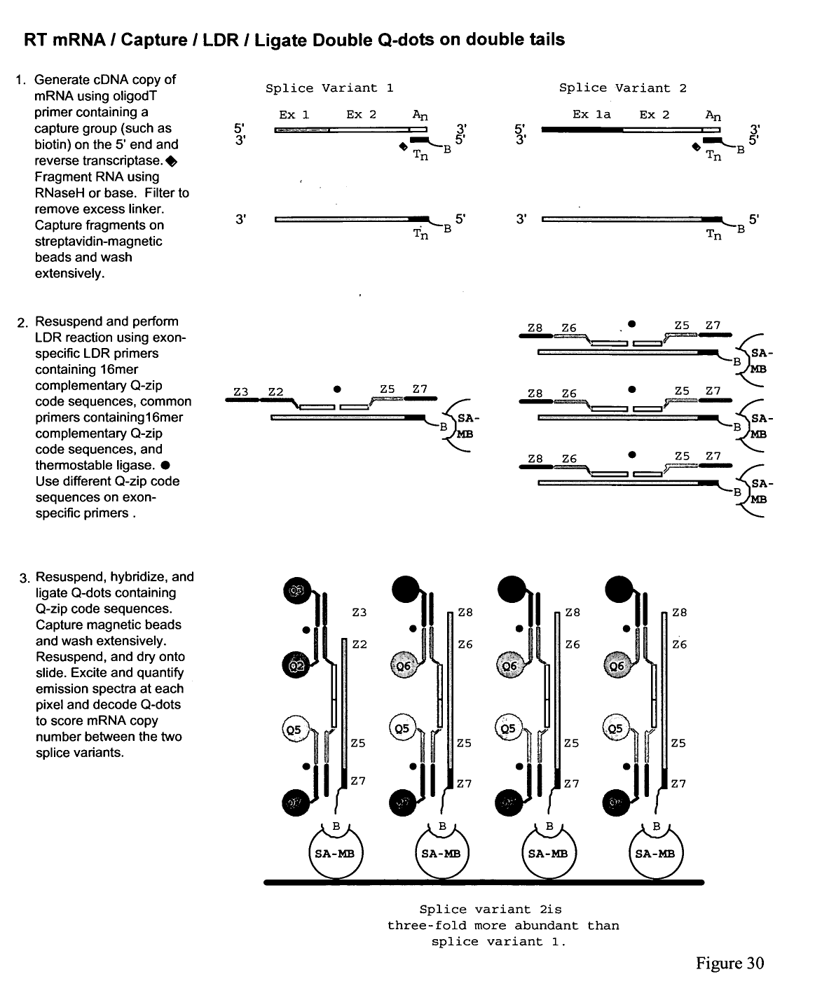

FIG. 30 is a schematic diagram of an LDR process, in accordance with the present invention, where ligated double Q-Zip portions containing Q-dots on double tails are used with microbead capture to distinguish and quantify splice variants on mRNA. FIG. 30 is similar to FIG. 29, but with the addition of ligating adjacent Q-Zip portions containing Q-dots in the last step. The detection results in FIG. 30 show an approximately three-fold greater amount of splice variant containing Ex1a over the splice variant containing Ex1 (indicated by a ratio of hybridized ligated Q-Zip portions containing Q-dots (Q8,Q6,Q5,Q7)/(Q3,Q2,Q5,Q7) of about three).

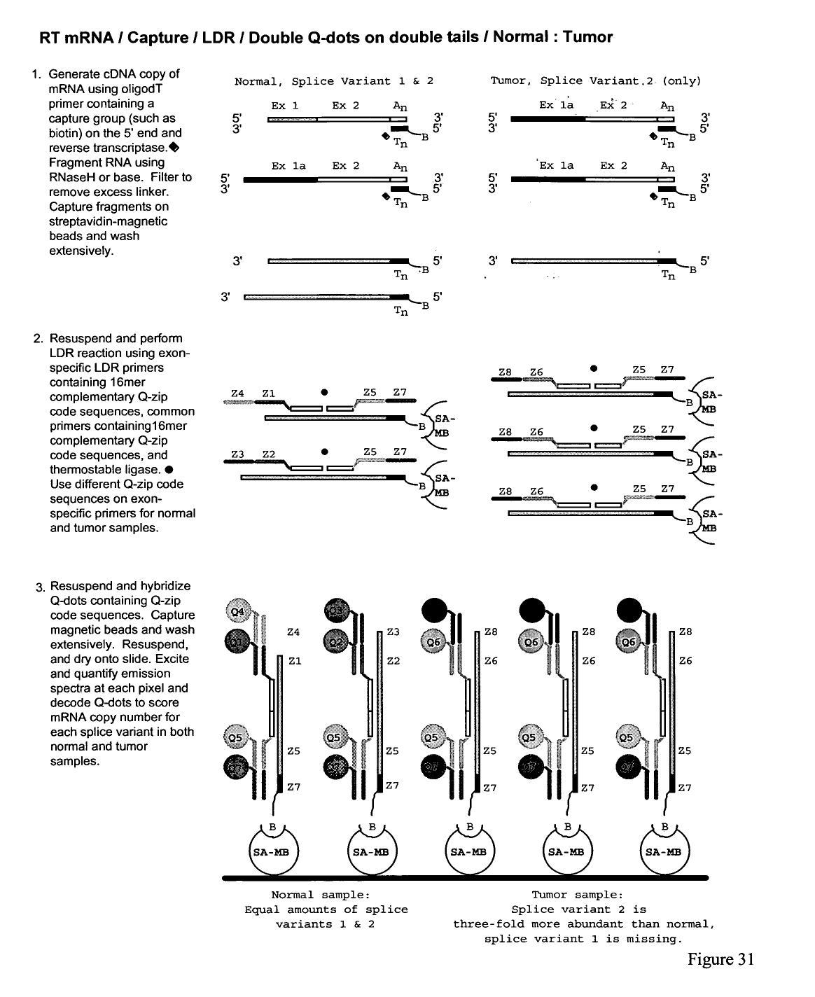

FIG. 31 is a schematic diagram of an LDR process, in accordance with the present invention, where double Q-Zip portions containing Q-dots on double tails are used with microbead capture to distinguish and quantify splice variants on mRNA in a tumor sample compared with a normal sample. This figure is similar to FIG. 29, except the Q-dot encryption is used to score splice variants in the tumor sample compared with the normal sample. cDNAs are captured on streptavidin-magnetic beads and washed extensively as described above. Tumor and normal samples are kept separate. In the initial LDR reaction, a given splice junction is interrogated with two allele-specific encrypted oligonucleotice probes in the normal sample in a first tube. The same splice junction is interrogated with two different allele-specific encrypted oligonucleotice probes in the tumor sample in a second tube. Although multiple different splice variants are detected in each tube, for simplicity only splice variants 1 and 2 are shown. After the ligase detection reaction, the ligation products on magnetic beads are combined in a single tube and washed extensively. The beads are resuspended and dried onto a slide. Emission spectra at each pixel are quantified to decode the presence of specific Q-dots, to score splice variants in the tumor compared to the normal sample.

The detection results in FIG. 31 show that the tumor demonstrates a loss of the Ex1 variant and an approximately three-fold higher expression of the Ex1a splicing variant. In FIG. 31, the ratio of hybridized Q-Zip portions containing Q-dots (Q1,Q5,Q5,Q7)/(Q4,Q1,Q5,Q7) is about zero, indicating loss of the Ex1 variant in the tumor. The ratio of hybridized Q-Zip portions containing Q-dots (Q8,Q6,Q5,Q7)/(Q3,Q2,Q5,Q7) is about three, indicating an approximately three-fold higher expression of the Ex1a splicing variant in the tumor.

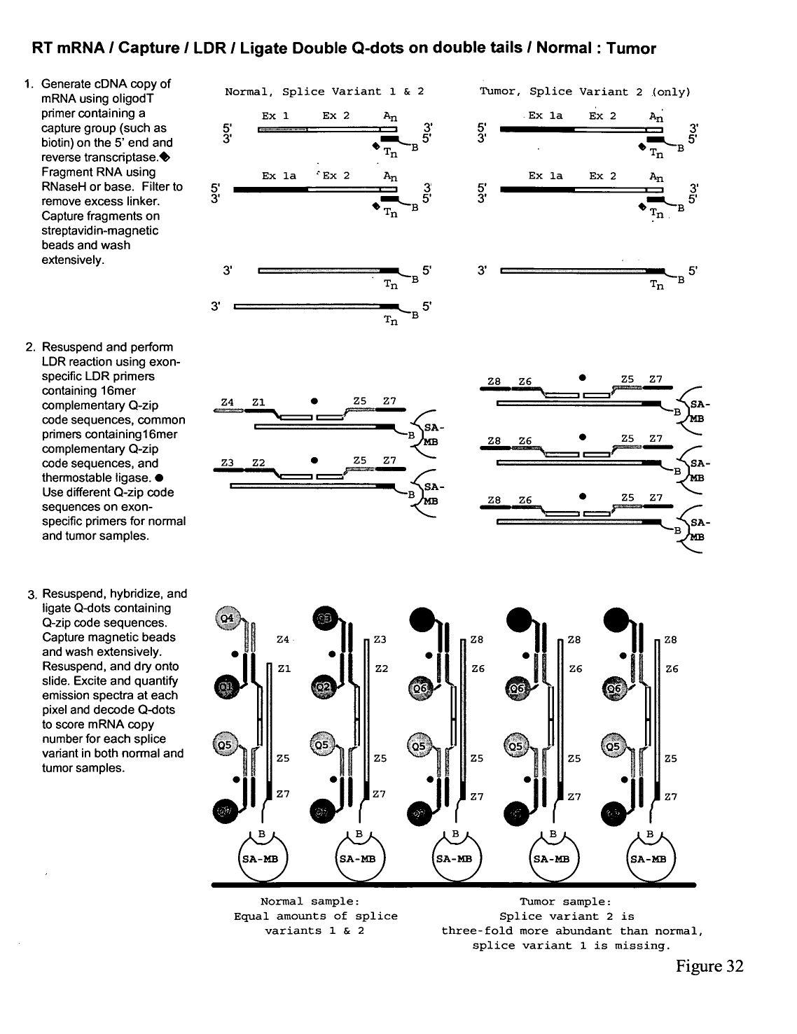

FIG. 32 is a schematic diagram of an LDR process, in accordance with the present invention, where ligated double Q-Zip portions containing Q-dots on double tails are used with microbead capture to distinguish and quantify splice variants on mRNA in a tumor sample compared with a normal sample. FIG. 32 is similar to FIG. 31, but with the addition of ligating adjacent Q-Zip portions containing Q-dots in the last step.

The detection results in FIG. 32 show that the tumor demonstrates a loss of the Ex1 variant and an approximately three-fold higher expression of the Ex1a splicing variant. In FIG. 32, the ratio of hybridized ligated Q-Zip portions containing Q-dots (Q1,Q5,Q5,Q7)/(Q4,Q1,Q5,Q7) is about zero, indicating loss of the Ex1 variant in the tumor. The ratio of hybridized ligated Q-Zip portions containing Q-dots (Q8,Q6,Q5,Q7)/(Q3,Q2,Q5,Q7) is about three, indicating an approximately three-fold higher expression of the Ex1a splicing variant in the tumor.

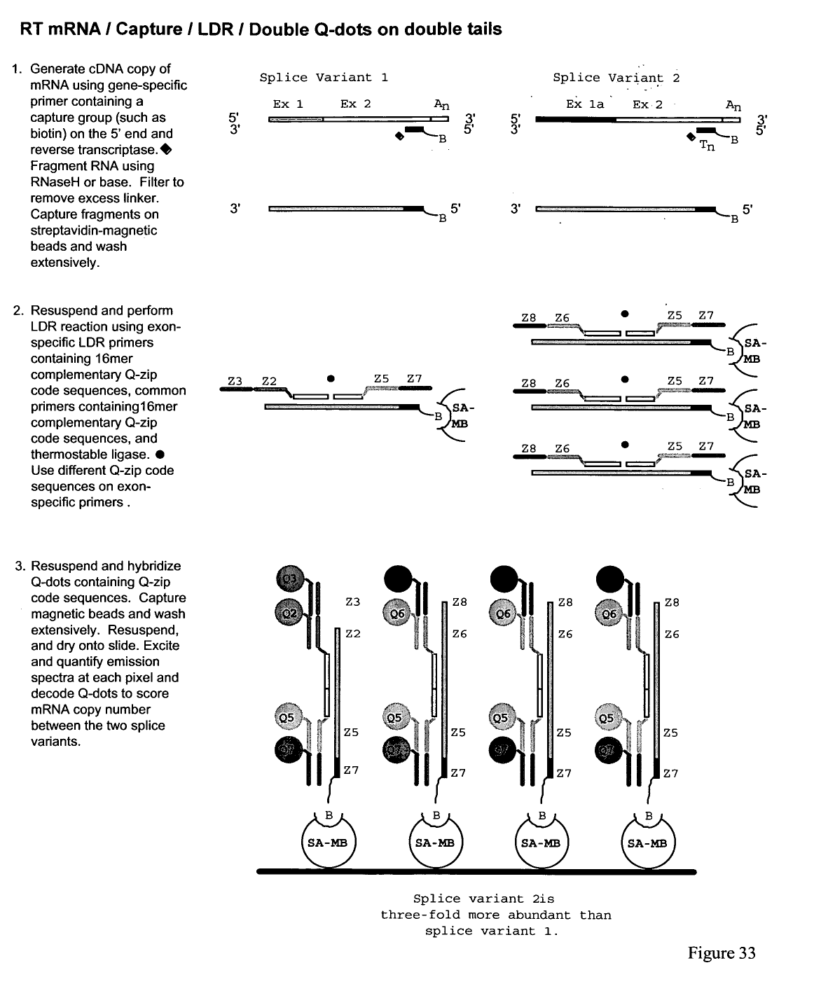

FIG. 33 is a schematic diagram of an LDR process, in accordance with the present invention, where double Q-Zip portions containing Q-dots on double tails are used with microbead capture to distinguish and quantify splice variants on mRNA. FIG. 33 is similar to FIG. 29, but with the use of gene specific primer(s) instead of oligo dT primers to generate the cDNA strand. The detection results in FIG. 33 show an approximately three-fold greater amount of splice variant containing Ex1a over the splice variant containing Ex1 (indicated by a ratio of hybridized Q-Zip portions containing Q-dots (Q8,Q6,Q5,Q7)/(Q3,Q2,Q5,Q7) of about three).

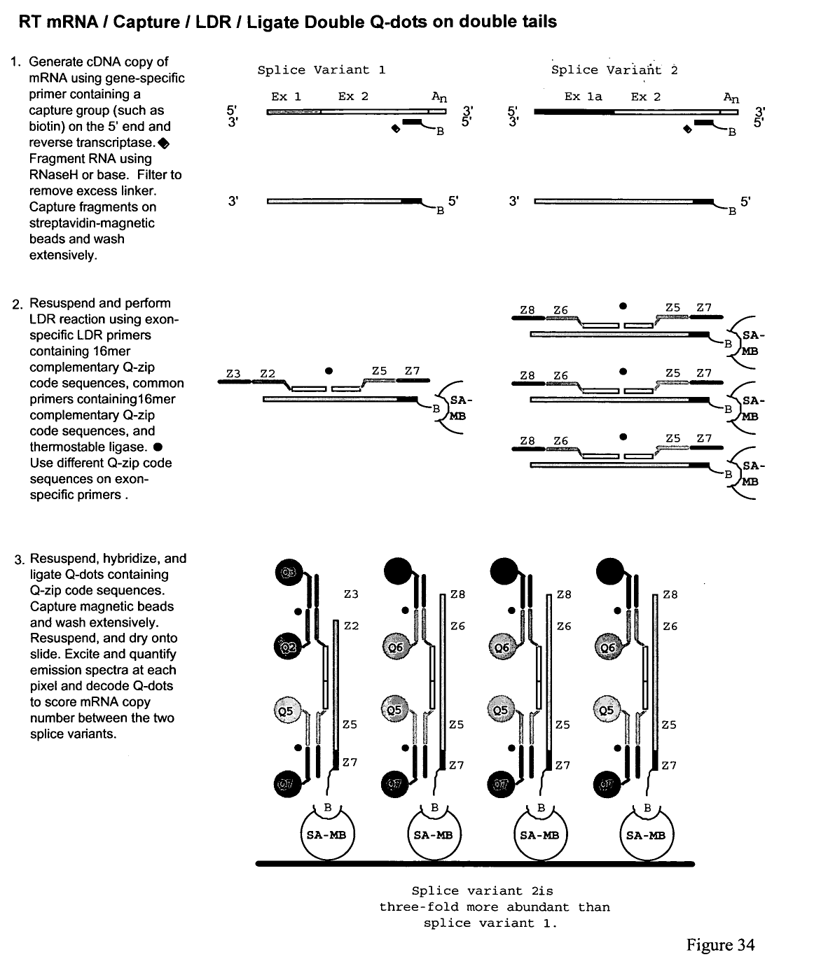

FIG. 34 is a schematic diagram of an LDR process, in accordance with the present invention, where ligated double Q-Zip portions containing Q-dots on double tails are used with microbead capture to distinguish and quantify splice variants on mRNA. FIG. 34 is similar to FIG. 33, but with the addition of ligating adjacent Q-Zip portions containing Q-dots in the last step. The detection results in FIG. 34 show an approximately three-fold greater amount of splice variant containing Ex1a over the splice variant containing Ex1 (indicated by a ratio of hybridized ligated Q-Zip portions containing Q-dots (Q8,Q6,Q5,Q7)/(Q3,Q2,Q5,Q7) of about three).

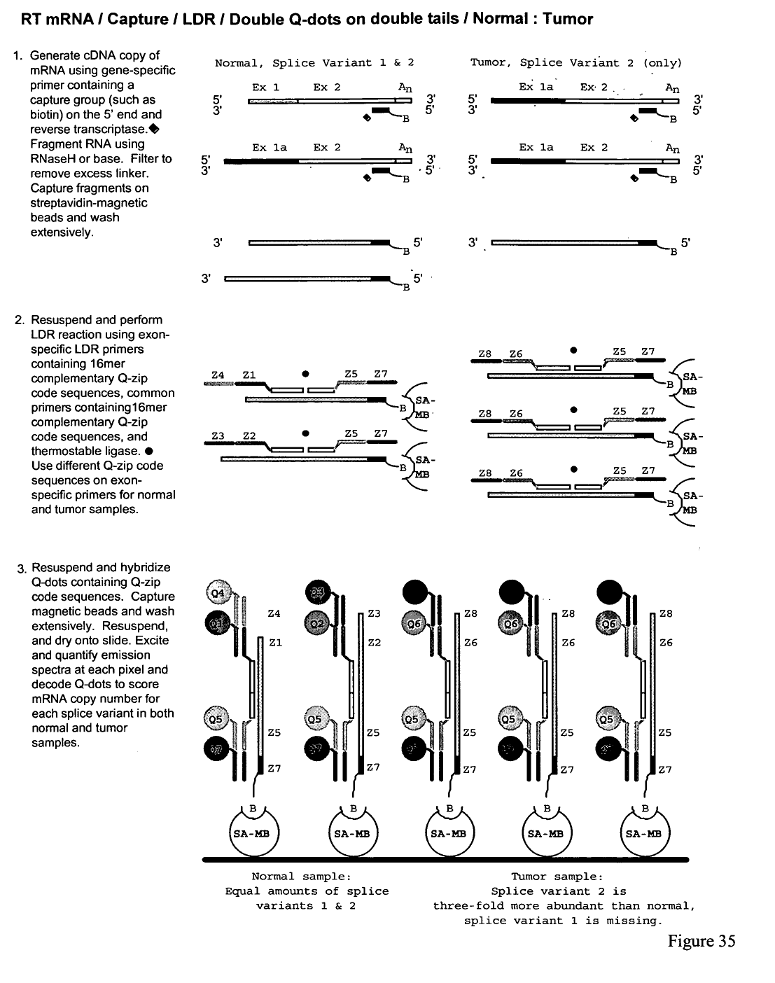

FIG. 35 is a schematic diagram of an LDR process, in accordance with the present invention, where double Q-Zip portions containing Q-dots on double tails are used with microbead capture to distinguish and quantify splice variants on mRNA in a tumor sample compared with a normal sample. FIG. 35 is similar to FIG. 31, but with the use of gene specific primer(s) instead of oligo dT primers to generate the cDNA strand.

The detection results in FIG. 35 show that the tumor demonstrates a loss of the Ex1 variant and an approximately three-fold higher expression of the Ex1 a splicing variant. In FIG. 35, the ratio of hybridized Q-Zip portions containing Q-dots (Q1,Q5,Q5,Q7)/(Q4,Q1,Q5,Q7) is about zero, indicating loss of the Ex1 variant in the tumor. The ratio of hybridized Q-Zip portions containing Q-dots (Q8,Q6,Q5,Q7)/(Q3,Q2,Q5,Q7) is about three, indicating an approximately three-fold higher expression of the Ex1a splicing variant in the tumor.

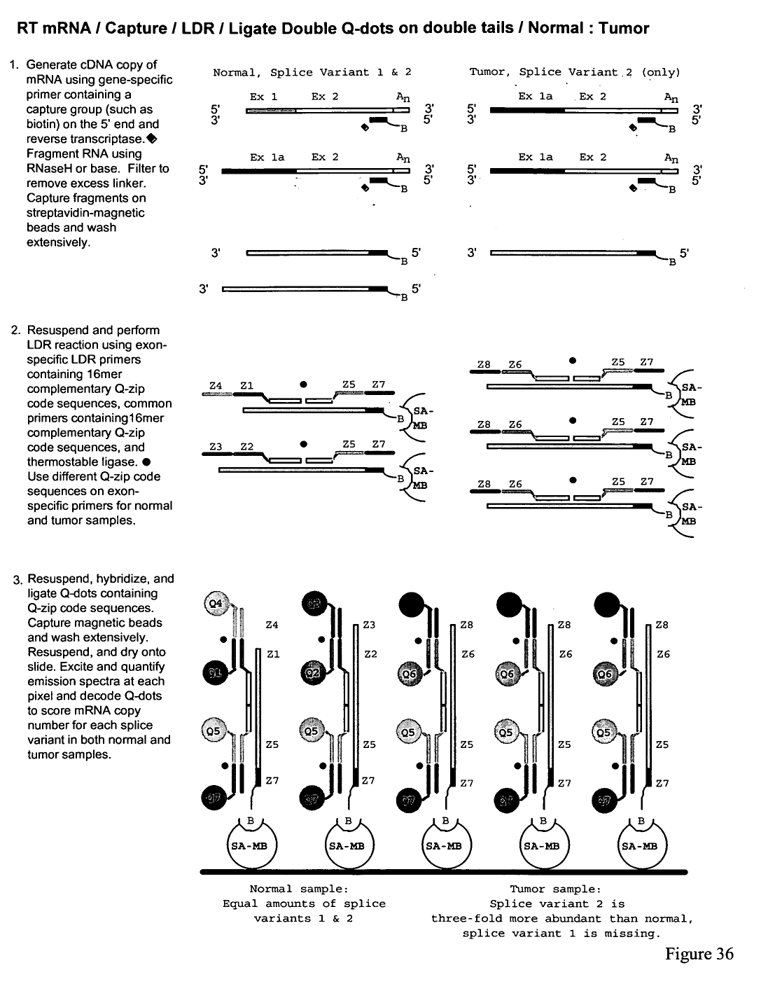

FIG. 36 is a schematic diagram of an LDR process, in accordance with the present invention, where ligated double Q-Zip portions containing Q-dots on double tails are used with microbead capture to distinguish and quantify splice variants on mRNA in a tumor sample compared with a normal sample. FIG. 36 is similar to FIG. 35, but with the addition of ligating adjacent Q-Zip portions containing Q-dots in the last step.

The detection results in FIG. 36 show that the tumor demonstrates a loss of the Ex1 variant and an approximately three-fold higher expression of the Ex1a splicing variant. In FIG. 36, the ratio of hybridized ligated Q-Zip portions containing Q-dots (Q1,Q5,Q5,Q7)/(Q4,Q1,Q5,Q7) is about zero, indicating loss of the Ex1 variant in the tumor. The ratio of hybridized ligated Q-Zip portions containing Q-dots (Q8,Q6,Q5,Q7)/(Q3,Q2,Q5,Q7) is about three, indicating an approximately three-fold higher expression of the Ex1a splicing variant in the tumor.

In carrying out the solid support capture, ligase detection reaction, detection probe hybridization, and detection scheme of the present invention, relative amounts of one or more of a plurality of target nucleic acid molecules in the test sample, differing by one or more single-base changes, insertions, deletions, or translocations or one or more target mRNA molecules differing by one or more splice site variables may be quantified. This is achieved by comparison with a reference sample having reference target nucleic acid molecules or reference target mRNA molecule.

This involves providing one or more secondary oligonucleotide probe sets, which differ from the primary oligonucleotide probe sets. Each of the secondary oligonucleotide probe sets are characterized by (a) a first oligonucleotide probe having a reference target-specific portion and a 5′ upstream portion complementary to one or more detection oligonucleotide probes and (b) a second oligonucleotide probe having a reference target-specific portion and a 3′ downstream portion complementary to one or more detection oligonucleotide probes. The oligonucleotide probes in a particular secondary oligonucleotide probe set are suitable for ligation together when hybridized to one another on a corresponding reference target nucleic acid molecule or reference target mRNA molecule, but have a mismatch which interferes with this ligation when hybridized to any other nucleic acid or mRNA molecule present in the reference sample. One or both oligonucleotide probes in the secondary oligonucleotide probe set contain one or more detection oligonucleotide probe-specific portions or their complements so that each secondary oligonucleotide probe set contains a unique set of one or more detection oligonucleotide probe-specific portions or their complements.

The reference sample, the one or more secondary oligonucleotide probe sets, and the ligase are blended to form a secondary ligase detection reaction mixture. The secondary ligase detection reaction mixture is then subjected to one or more ligase detection reaction cycles comprising a denaturation treatment and a hybridization treatment. In the denaturation treatment, any hybridized oligonucleotides are separated from reference target nucleic acid molecules or reference target mRNA molecules. In the hybridization treatment, the oligonucleotide probe sets hybridize in a base-specific manner to their respective reference sample target nucleic acid molecule or reference target mRNA molecule, if present in the sample, and ligate to one another to form a secondary ligation product containing (a) the reference sample-specific portions and (b) the one or more detection oligonucleotide probe-specific portions or their complements. The secondary ligation product for each secondary oligonucleotide probe set being distinguishable from other nucleic acid molecules in the secondary ligase detection reaction mixture. The secondary oligonucleotide probe sets may hybridize to nucleic acid molecules in the reference sample other than their respective reference target nucleic acid molecules or reference mRNA molecules but do not ligate together due to the presence of one or more mismatches and individually separate during the denaturation treatment.

The primary and secondary ligase detection reaction mixtures are blended after subjecting them to one or more ligase detection reaction cycles and before contacting. As a result, the blended primary and secondary ligase detection reaction mixtures are subjected to contacting and detecting. The relative amounts of the reporter labels on the primary and secondary ligation products are compared, to provide a quantitative measure of the relative level of the one or more target nucleic acid molecules or target mRNA molecules in the test sample compared with that of the reference target nucleic acid molecules or reference target mRNA molecules in the reference sample as a result of each different labeled ligation product having a unique encryption code. This aspect of the present invention is useful for quantifying, without limitation, gene copy number, mRNA copy number, or mRNA splice variant copy number.

Ligase Detection Reaction, Ligase Detection Reaction, Detection Probe Hybridization, and Detection Scheme

Another aspect of the present is directed to a method for identifying one or more target nucleic acid molecules differing by one or more single-base changes, insertions, deletions, or translocations in a plurality of nucleic acid molecules. This method includes ligase detection reaction, ligase detection reaction, detection probe hybridization, and detection.

The first ligase detection reaction phase, as described above, involves providing a test sample potentially containing one or more target nucleic acid molecules. Also provided are one or more primary oligonucleotide probe sets, each set characterized by (a) a first oligonucleotide probe, having a target-specific portion and a 5′ upstream portion containing one or more translational oligonucleotide probes or their complements, and (b) a second oligonucleotide probe, having a target-specific portion. The oligonucleotide probes in a particular primary oligonucleotide set are suitable for ligation together when hybridized to a corresponding target nucleic acid molecule, but have a mismatch which interferes with this ligation when hybridized to any other nucleic acid molecule present in the sample. Each probe set contains a unique combination of translational oligonucleotide probes or their complements.

The sample, the one or more primary oligonucleotide probe sets, and the ligase are blended to form a primary ligase detection reaction mixture. The primary ligase detection reaction mixture is subjected to one or more ligase detection reaction cycles. These cycles include a denaturation treatment and hybridization treatment. In the denaturation treatment, any hybridized oligonucleotides are separated from the target nucleic acid molecules. During the hybridization treatment, the oligonucleotide probes or the primary oligonucleotide probe sets hybridize in a base-specific manner to their respective target nucleic acid molecules, if present in the sample, and ligate to one another to form a primary ligation product. The primary ligation product contains (a) the translational oligonucleotide-specific portion or their complements and (b) the target-specific portions. The ligation product for each primary oligonculeotide probe set is distinguishable from other nucleic acids in the primary ligase detection reaction mixture by virtue of its containing a unique combination of translational oligonucleotide portions or their complements. The primary oligonucleotide probe sets may hybridize to nucleic acid molecules in the sample other than their respective target nucleic acid molecules but do not ligate together due to the presence of one or more mismatches and individually separate during the denaturation treatment.

For the second ligase detection phase, as substantially described above, one or more secondary oligonucleotide probe sets are provided. Each set is characterized by (a) a first oligonucleotide probe, having a translational oligonucleotide-specific portion and a 5′ upstream portion complementary to one or more detection oligonucleotide probe sequences and (b) a second oligonucleotide probe, having a translational oligonucleotide-specific portion, and a 3′ downstream portion complementary to one or more detection oligonucleotide probe sequences. The oligonucleotide probes in a particular secondary oligonucleotide probe set are suitable for ligation together when hybridized to a corresponding complement of a primary ligation product, but have a mismatch which interferes with this ligation when hybridized to any other nucleic acid molecule present in the sample. As a result, each secondary oligonucleotide probe set contains a unique set of 5′ upstream and 3′ downstream portions.

The primary ligation products, the plurality of secondary oligonucleotide probe sets, and the ligase are blended to form a secondary ligase detection reaction mixture. The secondary ligase detection reaction mixture is subjected to one ligase detection reaction cycle. Each cycle includes a denaturation treatment and a hybridization treatment. In the denaturation treatment, any hybridized oligonucleotide probes are separated from nucleic acid molecules to which they are hybridized. During the hybridization treatment, the secondary oligonucleotide probe sets hybridize in a base-specific manner to their corresponding primary ligation products, if present, and ligate to one another to form a secondary ligation product containing (a) the 5′ upstream portion complementary to one or more distinct oligonucleotide probe sequences, (b) the upstream translational oligonucleotide-specific portion, (c) the downstream translational oligonucleotide-specific portion, and (d) the 3′ downstream portion complementary to one or more distinct oligonucleotide probe sequences. The secondary oligonucleotide probe sets may hybridize to nucleic acid molecules other than their respective primary ligation products but do not ligate together due to the presence of one or more mismatches and individually separate during the denaturation treatment. Also provided are detection oligonucleotide probes which bind to the 5′ upstream portion and the 3′ downstream portion, where each detection oligonucleotide probe has a reporter label, so that each of the products has a unique detectable encryption code.

In the detection probe hybridization phase, as described above, the secondary ligation products and the detection oligonucleotide probes are contacted under conditions effective to permit hybridization of the detection oligonucleotide probes to the ligation products so that labeled, secondary ligation products are formed.

In the detection phase, as described above, the reporter labels on the labeled, secondary ligation products are detected. The presence of one or more target nucleic acid molecule in the sample is indicated, where sequences differing by one or more single-base changes, insertions, deletions, or translocations are discriminated from one another during the primary ligase detection reaction. The discriminated 30 sequences are detected as a result of each different labeled secondary ligation product having a unique encryption code with a different pattern of detectable emission spectra.

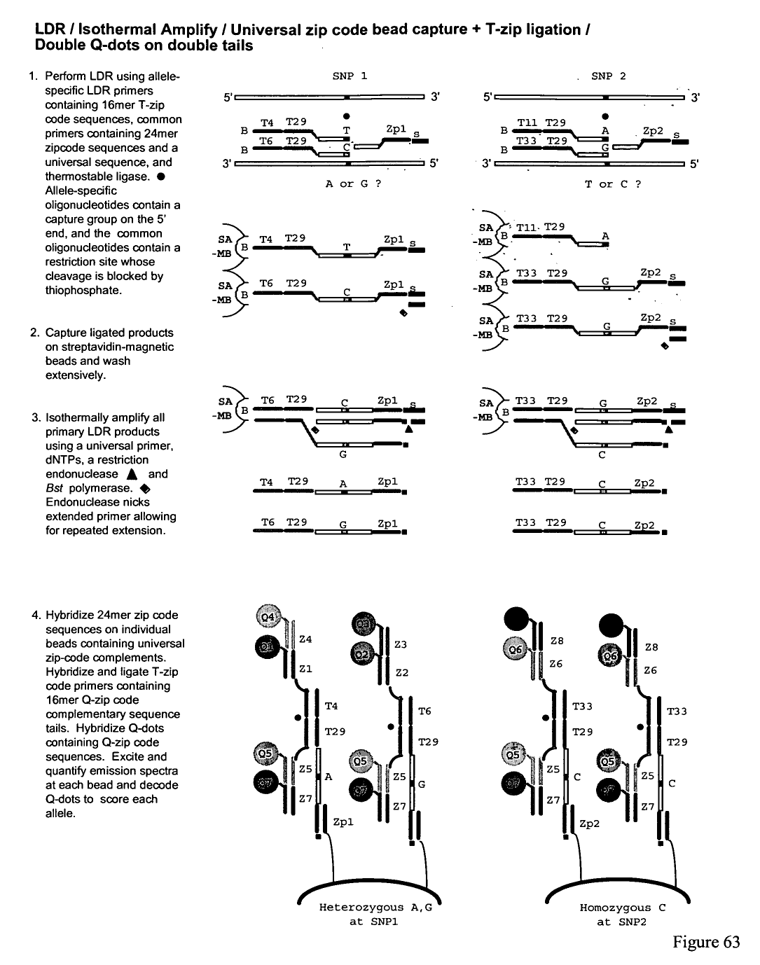

Examples of the ligase detection reaction, ligase detection reaction, detection probe hybridization, and detection scheme are shown in FIGS. 63-71.

FIG. 63 is a schematic diagram of an LDR process, in accordance with the present invention, where isothermal amplification and double Q-Zip portions containing Q-dots on double tails are used with T-Zip portion ligation and universal bead capture with addressable array-specific portions to distinguish single base differences in nucleic acid molecules. LDR is performed using allele-specific oligonucleotide probes containing 16mer T-Zip portions, common oligonucleotide probes containing 24mer addressable array-specific portions and a universal sequence, and a thermostable ligase. Allele-specific T-Zip portions (T4,T29), (T6,T29), (T11,T29), and (T33,T29) discriminate A, G, T, and C, respectively, on the target 3′→5′ nucleotide sequence. Allele-specific oligonucleotide probes contain a capture group on the 5′ end, and the common oligonucleotide probes contain a restriction site whose cleavage is blocked by a thiophosphate group at the scissile bond. Allele-specific oligonucleotide probes ligate to common oligonucleotide probes only when there is perfect complementarity at the junction. Subsequently, ligation products are captured on streptavidin-magnetic beads and washed extensively. All primary ligation products are amplified isothermally using a universal primer, dNTPs, a restriction endonuclease, and Bst polymerase. The universal primer contains the restriction endonuclease site and hybridizes to the ligation products. Once Bst polymerase extends this primer, the restriction endonuclease nicks this strand while extension continues. The ligation product strand is not cleaved as the thiophosphate containing strand is resistant. However, the nicked primer becomes a substrate for a new polymerase to bind and extend, displacing the previous strand. This isothermal amplification allows hundreds to thousands or more copies to accumulate in a linear fashion. Extension products are hybridized onto individual beads containing the universal 24-mer addressable array-specific portions. Pairs of T-Zip portions containing 16mer Q-Zip portions are hybridized onto the extension products and ligated together. T-Zip ligation products on the beads are then hybridized with Q-Zip portions containing Q-dots. Emission spectra at each address are quantified to decode the presence of specific Q-dots, to score each allele.

The detection results in FIG. 63 show that the sample is heterozygous A, G at SNP1 and homozygous C at SNP2. The A allele is indicated by the presence of Q-Zip portions containing Q-dots (Q4,Q1,Q5,Q7) at address Zp1. The G allele is indicated by the presence of Q-Zip portions containing Q-dots (Q3,Q2,Q5,Q7) at address Zp1. The C allele is indicated by the presence of Q-Zip portions containing Q-dots (Q8,Q6,Q5,Q7) at address Zp2.

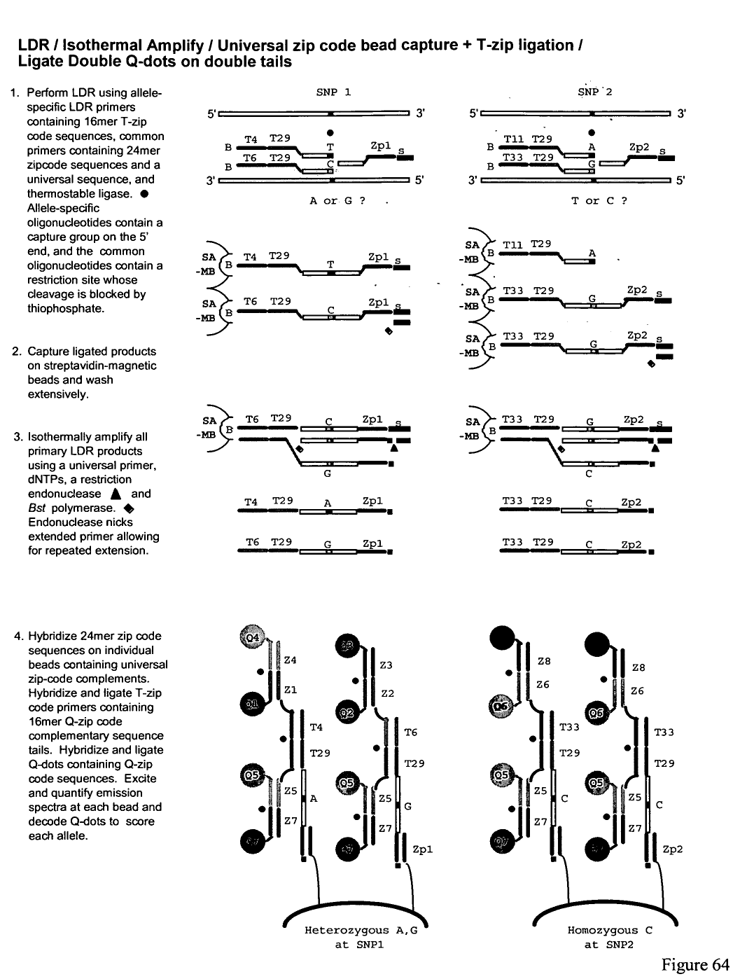

FIG. 64 is a schematic diagram of an LDR process, in accordance with the present invention, where isothermal amplification and ligated double Q-Zip portions containing Q-dots on double tails are used with T-Zip portion ligation and universal addressable array-specific portions to distinguish single base differences in nucleic acid molecules. FIG. 64 is similar to FIG. 63, but with the addition of ligating adjacent Q-Zip portions containing Q-dots in the last step.

The detection results in FIG. 64 show that the sample is heterozygous A, G at SNP1 and homozygous C at SNP2. The A allele is indicated by the presence of ligated Q-Zip portions containing Q-dots (Q4,Q1,Q5,Q7) at address Zp1. The G allele is indicated by the presence of ligated Q-Zip portions containing Q-dots (Q3,Q2,Q5,Q7) at address Zp1. The C allele is indicated by the presence of ligated Q-Zip portions containing Q-dots (Q8,Q6,Q5,Q7) at address Zp2.

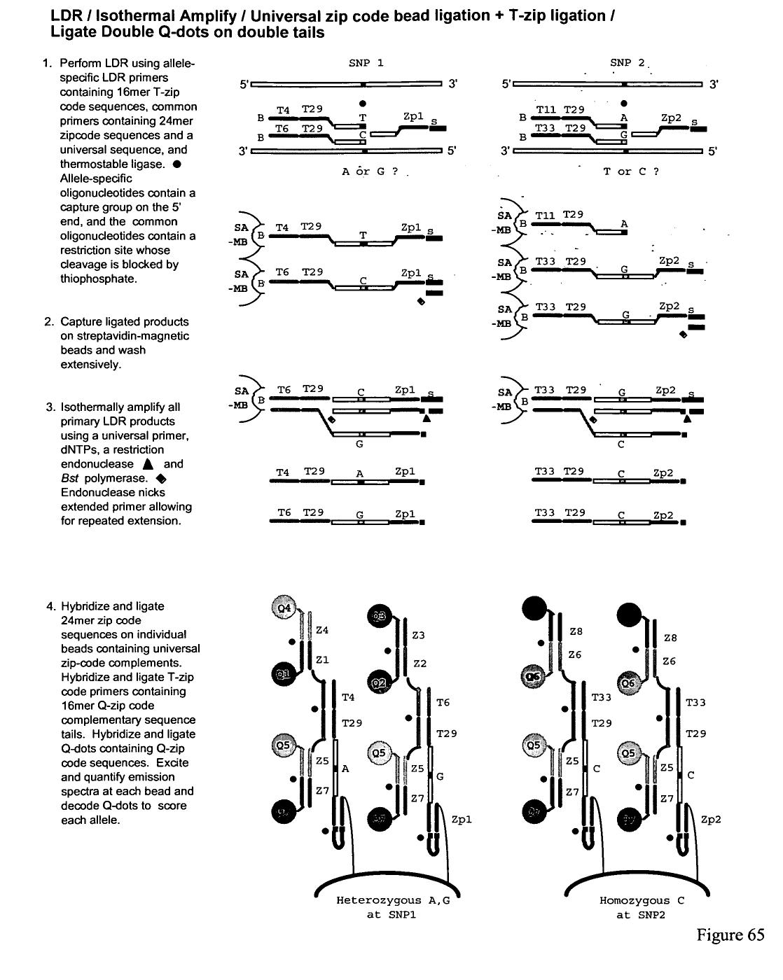

FIG. 65 is a schematic diagram of an LDR process, in accordance with the present invention, where isothermal amplification and ligated double Q-Zip portions containing Q-dots on double tails are used with T-Zip portions and ligation to universal capture probes with addressable array-specific portions on beads to distinguish single base differences in nucleic acid molecules. FIG. 65 is similar to FIG. 64, but with the addition of ligating the captured ligation products to capture probes on the beads prior to or during when the T-Zip portions are ligated. This may be followed by a stringent wash step. Subsequently, adjacent Q-Zip portions containing Q-dots are ligated in the last step.

The detection results in FIG. 65 show that the sample is heterozygous A, G at SNP1 and homozygous C at SNP2. The A allele is indicated by the presence of ligated Q-Zip portions containing Q-dots (Q4,Q1,Q5,Q7) at address Zp1. The G allele is indicated by the presence of ligated Q-Zip portions containing Q-dots (Q3,Q2,Q5,Q7) at address Zp1. The C allele is indicated by the presence of ligated Q-Zip portions containing Q-dots (Q8,Q6,Q5,Q7) at address Zp2.

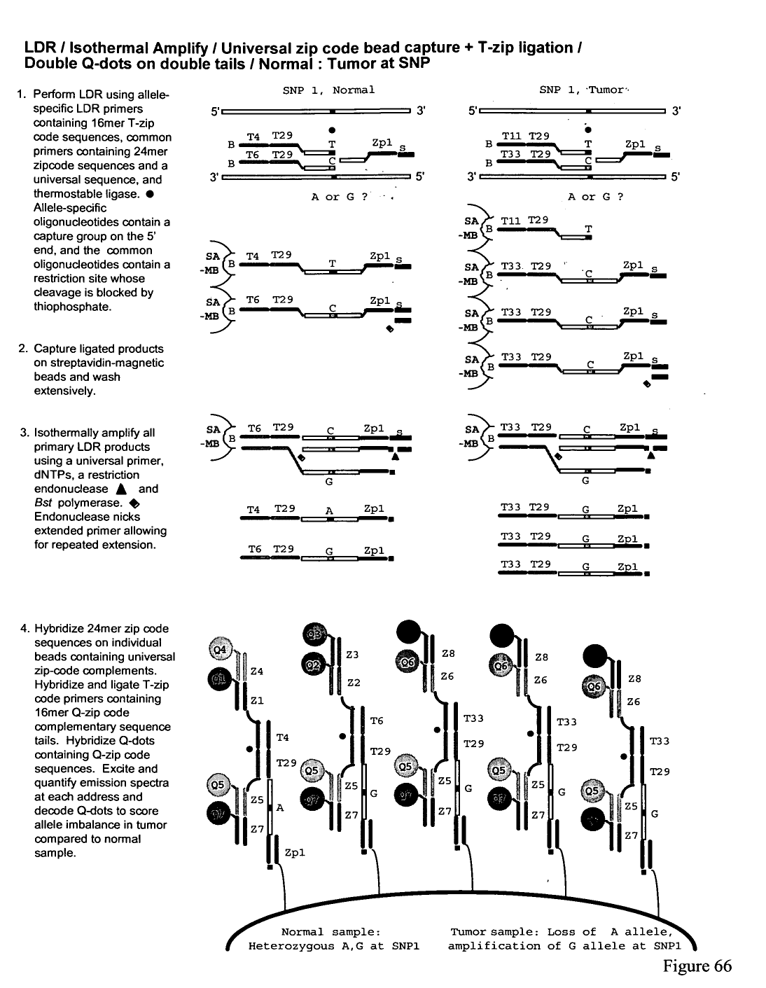

FIG. 66 is a schematic diagram of an LDR process, in accordance with the present invention, where isothermal amplification and ligated double Q-Zip portions containing Q-dots on double tails are used with T-Zip portion ligation to universal capture probes with addressable array-specific portions to score allele imbalance in a tumor sample compared with a normal sample. FIG. 66 is similar to FIG. 63, except the Q-dot encryption is used to score allele imbalance in a tumor sample compared with the normal sample. In the initial LDR reaction a given single base difference is interrogated with two allele-specific encrypted oligonucleotide probes in the normal sample in a first tube. The same single base difference is interrogated with two different allele-specific encrypted oligonucleotide probes in the tumor sample in a second tube. Although multiple different single base differences are detected in each tube, for simplicity only SNP1 is shown. The ligation products are combined in a single tube and the ligation product capture, isothermal amplification, and subsequent procedure are the same as described in FIG. 63. Emission spectra at each bead are quantified to decode the presence of specific Q-dots, to score allele imbalance in the tumor compared to the normal sample.

The detection results in FIG. 66 show that at SNP1 the normal sample is heterozygous A, G and the tumor sample demonstrates a loss of the A allele and amplification of the G allele. In FIG. 66, the ratio of Q-Zip portions containing Q-dots (Q1,Q5,Q5,Q7)/(Q4,Q1,Q5,Q7) at address Zp1 is about zero, indicating loss of the A allele in the tumor. The ratio of Q-Zip portions containing Q-dots (Q8,Q6,Q5,Q7)/(Q3,Q2,Q5,Q7) at address Zp1 is about three, indicating an approximately three-fold amplification of the G allele in the tumor.

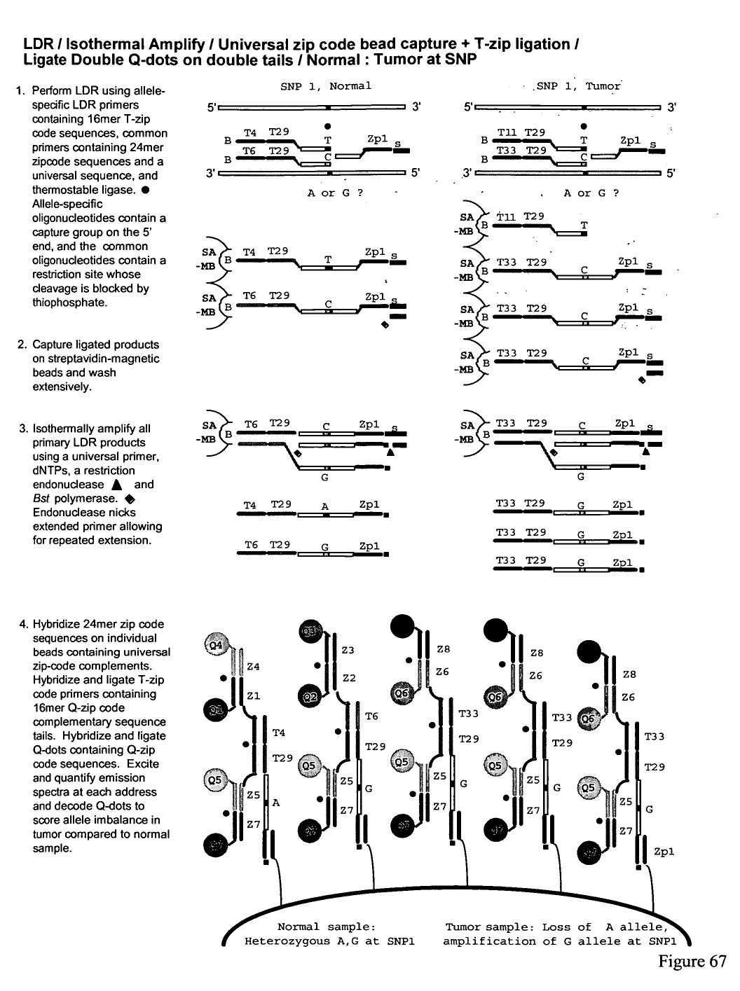

FIG. 67 is a schematic diagram of an LDR process, in accordance with the present invention, where isothermal amplification and double Q-Zip portions containing Q-dots on double tails are used with T-Zip portion ligation and universal bead capture with addressable array-specific portions to score allele imbalance in a tumor sample compared with a normal sample. FIG. 67 is similar to FIG. 66, but with the addition of ligating adjacent Q-Zip portions containing Q-dots in the last step.

The detection results in FIG. 67 show that at SNP1 the normal sample is heterozygous A, G and the tumor sample demonstrates a loss of the A allele and amplification of the G allele. In FIG. 67, the ratio of ligated Q-Zip portions containing Q-dots (Q1,Q5,Q5,Q7)/(Q4,Q1,Q5,Q7) at address Zp1 is about zero, indicating loss of the A allele in the tumor. The ratio of ligated Q-Zip portions containing Q-dots (Q8,Q6,Q5,Q7)/(Q3,Q2,Q5,Q7) at address Zp1 is about three, indicating an approximately three-fold amplification of the G allele in the tumor.

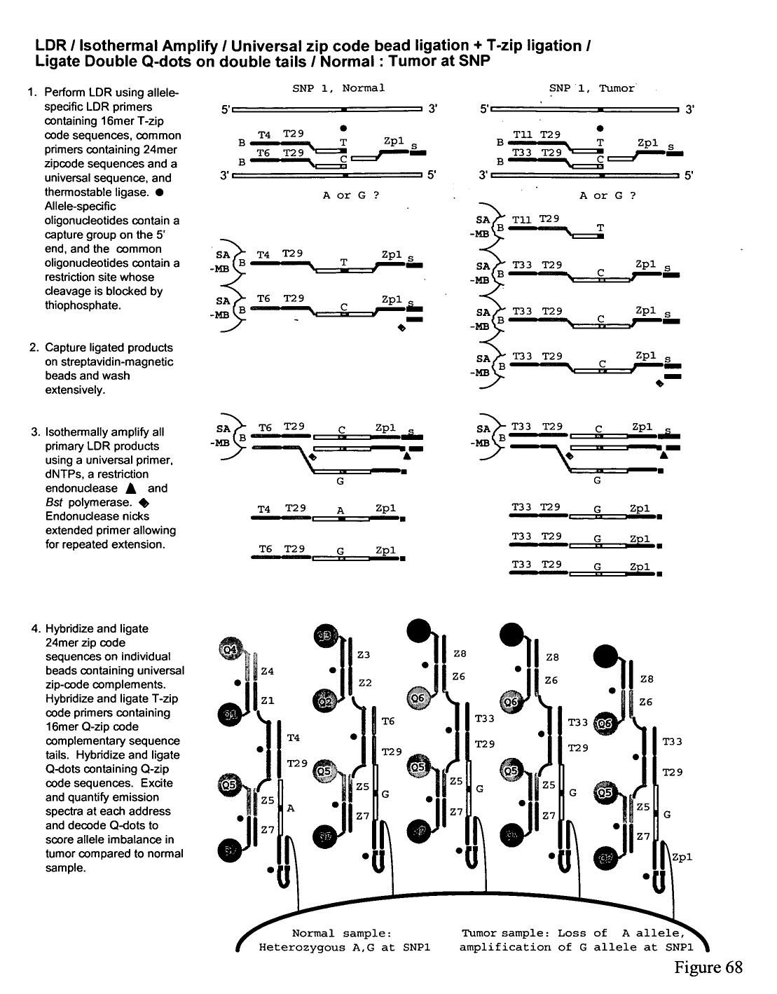

FIG. 68 is a schematic diagram of an LDR process, in accordance with the present invention, where isothermal amplification and ligated double Q-Zip portions containing Q-dots on double tails are used with T-Zip portion ligation and ligation to universal capture probes with addressable array-specific portions on beads to score allele imbalance in a tumor sample compared with a normal sample. FIG. 68 is similar to FIG. 67, but with the addition of ligating the captured ligation products to capture probes on the beads prior to or at the same time as the T-Zip portions are ligated. This may be followed by a stringent wash step. Subsequently, adjacent Q-Zip portions containing Q-dots are ligated in the last step.

The detection results in FIG. 68 show that at SNP1 the normal sample is heterozygous A, G and the tumor sample demonstrates a loss of the A allele and amplification of the G allele. In FIG. 68 the ratio of ligated Q-Zip portions containing Q-dots (Q1,Q5,Q5,Q7)/(Q4,Q1,Q5,Q7) at address Zp1 is about zero, indicating loss of the A allele in the tumor. The ratio of ligated Q-Zip portions containing Q-dots (Q8,Q6,Q5,Q7)/(Q3,Q2,Q5,Q7) at address Zp1 is about three, indicating an approximately three-fold amplification of the G allele in the tumor.

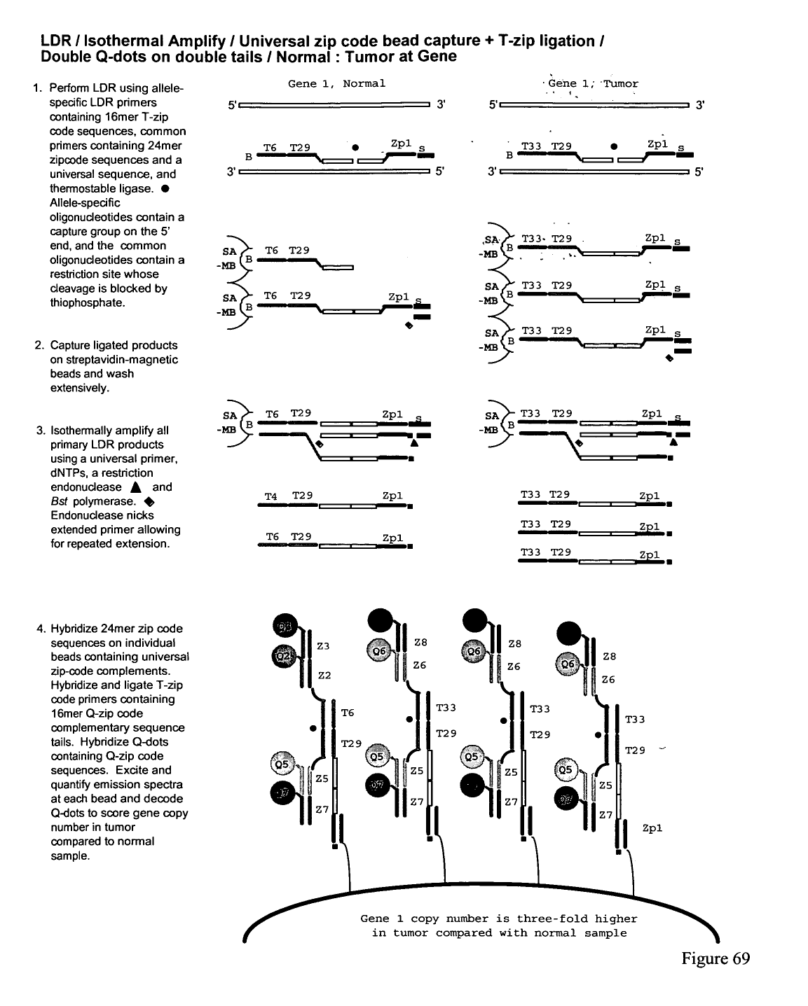

FIG. 69 is a schematic diagram of an LDR process, in accordance with the present invention, where isothermal amplification and double Q-Zip portions containing Q-dots on double tails are used with T-Zip portion ligation and universal bead capture with addressable array-specific portions to score gene copy number in a tumor sample compared with a normal sample. FIG. 69 is similar to FIG. 63, except the Q-dot encryption is used to score gene copy number in a tumor sample compared with the normal sample. In the initial LDR reaction, a given gene is interrogated with an allele-specific encrypted oligonucleotide probes in the normal sample in a first tube. The same gene is interrogated with a different allele-specific encrypted oligonucleotide probes in the tumor sample in a second tube. Although multiple different genes are detected in each tube, for simplicity only gene 1 is shown. The ligation products are combined in a single tube and the ligation product capture, isothermal amplification, and subsequent procedure are the same as described above. Emission spectra at each address are quantified to decode the presence of specific Q-dots, to score gene copy number in the tumor compared to the normal sample. The detection results in FIG. 69 show an approximately three-fold amplification of gene 1 in the tumor sample (indicated by a ratio of Q-Zip portions containing Q-dots (Q8,Q6,Q5,Q7)/(Q3,Q2,Q5,Q7) at address Zp1 of about three).

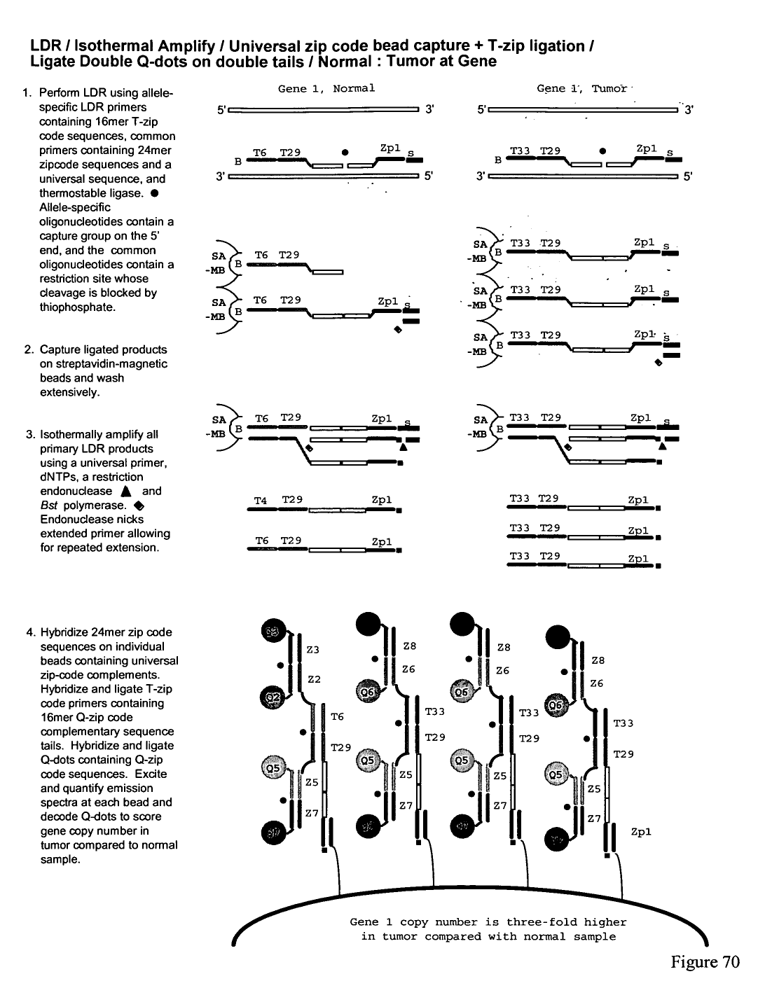

FIG. 70 is a schematic diagram of an LDR process, in accordance with the present invention, where isothermal amplification and ligated double Q-Zip portions containing Q-dots on double tails are used with T-Zip portion ligation and universal bead capture with addressable array-specific portions to score gene copy number in a tumor sample compared with a normal sample. FIG. 70 is similar to FIG. 69, but with the addition of ligating adjacent Q-Zip portions containing Q-dots in the last step. The detection results in FIG. 70 show an approximately three-fold amplification of gene 1 in the tumor sample (indicated by a ratio of ligated Q-Zip portions containing Q-dots (Q8,Q6,Q5,Q7)/(Q3,Q2,Q5,Q7) at address Zp1 of about three).

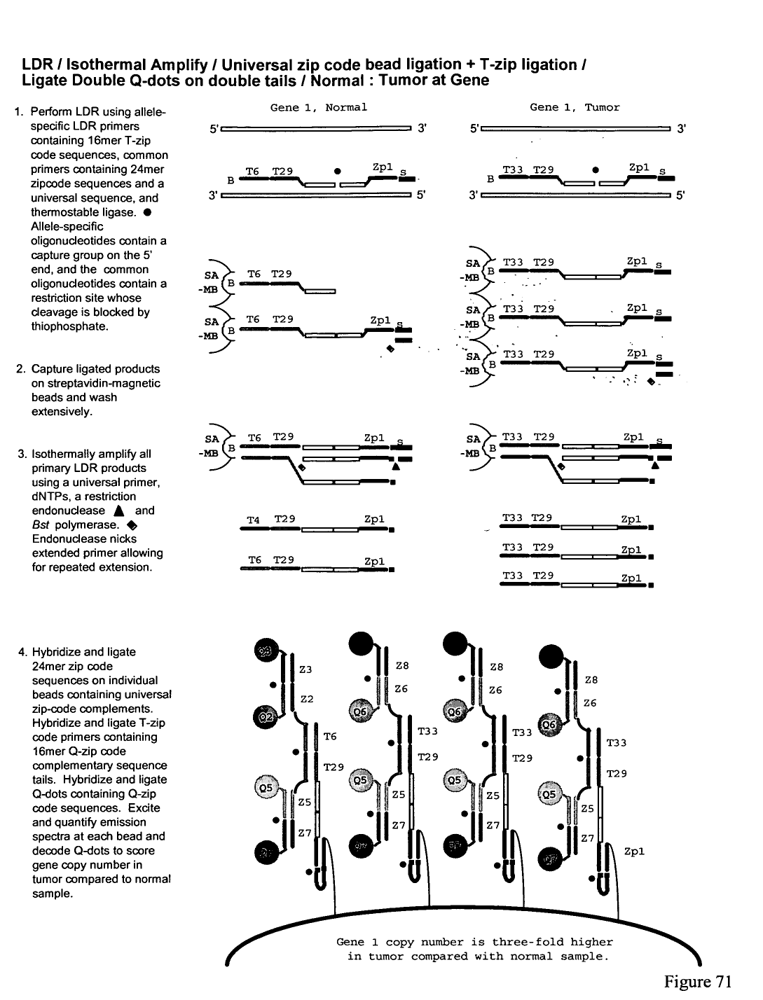

FIG. 71 is a schematic diagram of an LDR process, in accordance with the present invention, where isothermal amplification and ligated double Q-Zip portions containing Q-dots on double tails are used with T-Zip portion ligation and ligation to universal capture probes on beads to score gene copy number in a tumor sample compared with a normal sample. FIG. 71 is similar to FIG. 70, but with the addition of ligating the captured ligation products to the beads prior to or during the same step that the T-Zip portions are ligated to the ligation product. This may be followed by a stringent wash step. Subsequently, adjacent Q-Zip portions containing Q-dots are ligated in the last step. The detection results in FIG. 71 show an approximately three-fold amplification of gene 1 in the tumor sample (indicated by a ratio of ligated Q-Zip portions containing Q-dots (Q8,Q6,Q5,Q7)/(Q3,Q2,Q5,Q7) at address Zp1 of about three).

In carrying out the ligase detection reaction, ligase detection reaction, detection probe hybridization, and detection scheme of the present invention, relative amounts of one or more of a plurality of target nucleic acid molecules in the test sample, differing by one or more single-base changes, insertions, deletions, or translocations, may be quantified by comparison with a reference sample having reference target nucleic acid molecules.

This involves providing one or more reference oligonucleotide probe sets, which differ from the primary oligonucleotide probe sets, where each of the reference oligonucleotide probe sets are characterized by (a) a first oligonucleotide probe having a reference target-specific portion and a 5′ upstream portion containing one or more translational oligonucleotide probes or their complements and (b) a second oligonucleotide probe having a reference target-specific portion. The oligonucleotide probes in a particular reference oligonucleotide probe set are suitable for ligation together when hybridized to one another on a corresponding reference target nucleic acid molecule, but have a mismatch which interferes with this ligation when hybridized to any other nucleic acid molecule present in the reference sample. The reference sample, the one or more reference oligonucleotide probe sets, and the ligase are blended to form a reference ligase detection reaction mixture. The reference ligase detection reaction mixture is then subjected to one or more ligase detection reaction cycles comprising a denaturation treatment and a hybridization treatment. In the denaturation treatment, any hybridized oligonucleotides are separated from reference target nucleic acid molecules. During the hybridization treatment, the reference oligonucleotide probe sets hybridize in a base-specific manner to their respective reference sample target nucleic acid molecule, if present in the sample, and ligate to one another to form a reference ligation product sequence containing (a) the reference sample-specific portions and (b) the 5′ upstream portion containing one or more translation oligonucleotide probes or their complements. The reference ligation product for each reference oligonucleotide probe set is distinguishable from other nucleic acid molecules in the reference ligase detection reaction mixture. The reference oligonucleotide probe sets may hybridize to nucleic acid molecules in the reference sample other than their respective reference target nucleic acid molecules but do not ligate together due to the presence of one or more mismatches and individually separate during the denaturation treatment.

The primary and reference ligase detection reaction mixtures are blended after subjecting them to one or more ligase detection reaction cycles and before blending the primary ligation products, the plurality of secondary oligonucleotide probe sets, and the ligase, subjecting the secondary ligase detection reaction mixture to one or more ligase detection reaction cycles, contacting the secondary ligation products and the detection oligonucleotide probes, and detecting.

The relative amounts of the reporter labels on the secondary and reference ligation products are compared, to provide a quantitative measure of the relative level of the one or more target nucleic acid molecules in the test sample compared with the reference sample as a result of each different labeled ligation product having a unique encryption code.

Whole Genome Amplification Scheme

Another aspect of the present invention relates to a method of generating a linearly amplified representation of a whole genome. This involves providing genomic DNA molecules and subjecting the genomic DNA molecules to enzymatic digestion with a first restriction endonuclease to produce a degenerate oligonucleotide fragment with a degenerate overhang. A hairpin linker is provided which contains an overhang complementary to the degenerate overhang and a modification complementing the 5′ end of the degenerate oligonucleotide fragment within a second restriction site. The modification blocks restriction endonuclease cleavage on the 5′ side, but not the 3′ side, of the degenerate oligonucleotide fragment. The enzymatic digestion step is carried out in the presence of the hairpin linker, a ligase, and a first restriction endonuclease, under conditions effective to permit cleavage of genomic DNA molecules and ligation of the hairpin linker onto ends of the degenerate oligonucleotide fragments. Unligated linkers are then removed. Also provided are a second restriction endonuclease and a processive DNA polymerase with strand-displacement activity. The enzymatically digested genomic DNA molecules, the second restriction endonuclease, and the polymerase are blended to form a representational genome amplification mixture. The representational genome amplification mixture is incubated under conditions effective to permit the second restriction endonuclease to nick the hairpin DNA on its unmodified strand and the polymerase to extend the degenerate oligonucleotide fragments at their free 3′ ends. As the polymerase extends and displaces the pre-existing strand, it reforms the second restriction site allowing for repeated nicking/polymerase extension and linear amplification of a representation of the whole genome.

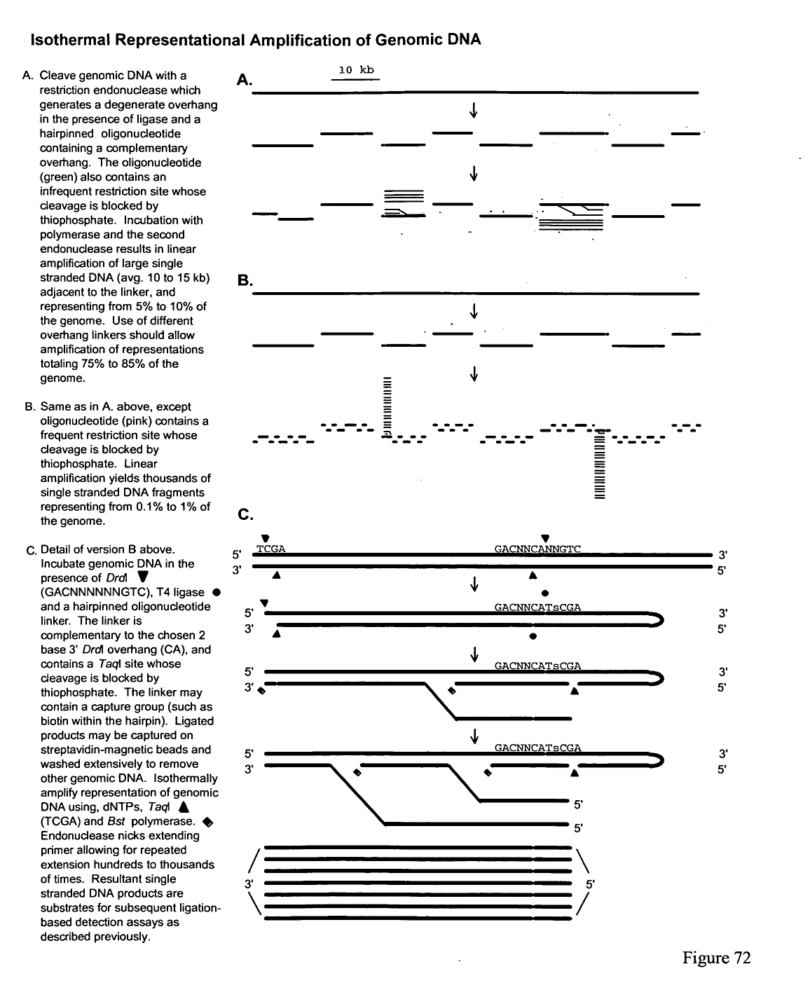

FIGS. 72A-C are flow diagrams showing isothermal representational amplification of genomic DNA. Two approaches for generating single stranded DNA representations of genomic DNA, suitable for subsequent ligation-based detection assays, are shown. Genomic DNA is cleaved with a restriction endonuclease that has an interrupted palindrome recognition sequence which generates a degenerate overhang in the presence of ligase and a hairpinned oligonucleotide containing a complementary overhang. Examples of such restriction enzymes include, without limitation, AlwNI (CAGNNN↓CTG), BglI (GCCNNNN↓NGGC (SEQ ID NO: 38883)), BstAPI (GCANNNN↓NTGC (SEQ ID NO: 38884)), BstXI (CCANNNNN↓NTGG (SEQ ID NO: 38885)), DrdI (GACNNNN↓NNGTC (SEQ ID NO: 38886)), PflMI (CCANNNN↓NTGG (SEQ ID NO: 38887)), and SfiI (GGCCNNNN↓NGGCC (SEQ ID NO: 38888)). The reaction is driven to completion, because genomic DNA that religates also reforms the recognition sequence and is recleaved. However, the site is not regenerated when a genomic end ligates to the linker. Since degenerate ends are non-palindromic, the linker does not form dimers. The oligonucleotide shown in FIG. 72A also contains an infrequent restriction site, such as SfiI (GGCCNNNN↓NGGCC (SEQ ID NO: 38888)) or BssHI (G↓CGCGC), whose cleavage is blocked by thiophosphate. Incubation with polymerase and the second endonuclease results in linear amplification of large single-stranded DNA (avg. 10 kb to 15 kb) adjacent to the linker, and representing from 5% to 10% of the genome. Use of different overhang linkers should allow amplification of representations totaling 75% to 85% of the genome.

The oligonucleotide linker shown in FIG. 72B contains a frequent restriction site whose cleavage is blocked by thiophosphate. Linear isothermal amplification yields thousands of single stranded DNA fragments representing from 0.1% to 1% of the genome.

As shown in FIG. 72C, genomic DNA is incubated in the presence of DrdI (GACNNNN↓NNGTC (SEQ ID NO: 38886)), T4 ligase, and a hairpinned oligonucleotide linker. The linker is complementary to the chosen 2 base 3′ DrdI overhang (CA), and contains a TaqI site whose cleavage is blocked by thiophosphate. The linker may contain a capture group (such as biotin within the hairpin). Ligation products may be captured on streptavidin-magnetic beads and washed extensively to remove other genomic DNA. Representation of genomic DNA may be isothermally amplified using dNTPs, TaqI (T→CGA) and Bst polymerase. Endonuclease nicks, shown in FIG. 72C, extend the primer allowing for repeated extension hundreds to thousands of times. The resultant single-stranded DNA products are substrates for subsequent ligation-based detection assays as described previously.

Designing a Plurality of Detection Oligonucleotide Probes

Another aspect of the present invention relates to a method of designing a plurality of labeled detection oligonucleotide probes for use in combinations of one to four or more probes to identify or quantify complementary sequences which will hybridize with little mismatch. The plurality labeled oligonucleotide probes have melting temperatures within a narrow range. This method involves providing a first set of a plurality of tetramers of four nucleotides linked together, where (1) each tetramer within the set differs from all other tetramers in the set by at least two nucleotide bases, (2) no two tetramers within a set are complementary to one another, (3) no tetramers within a set are palindromic or dinucleotide repeats, and (4) no tetramer within a set has less than one or more than three G and C nucleotides. Groups of 2 to 4 tetramers from the first set are linked together to form a collection of multimer units. All multimer units formed from the same tetramer and all multimer units having a melting temperature in ° C. of less than 6 times the number of tetramers forming a multimer unit removed from the collection of multimer units, to form a modified collection of multimer units. A second collection of multimer units are selected from the modified collection of multimer units so that no consecutive tetramer pair is used twice. 1 or 2 tetramers are added to either or both ends of the second collection of multimers to generate a new set of modified multimer units with higher melting temperatures, so that no consecutive tetramer pair is used twice. The new set of modified multimer units are arranged in a list in order of melting temperature. Units having a melting temperature in ° C. of less than 12 times the number of tetramers and more than 18 times the number of tetramers are removed from the set of modified multimer units to form a further collection of multimer units. Reporter labels are linked to the further collection of multimer units, to form labeled detection oligonucleotide probes.

The present invention also relates to a collection of labeled detection oligonucleotide probes. These probes include a collection of detection oligonucleotide probes to which complementary oligonucleotide probes will hybridize, within a narrow temperature range of greater than 24° C. with little mismatch. The oligonucleotide probes are formed from sets of tetramers where (1) each tetramer within the set differs from all other tetramers in the set by at least two nucleotide bases, (2) no two tetramers within a set are complementary to one another, (3) no tetramers within a set are palindromic or dinucleotide repeats, and (4) no tetramer within a set has less than one or more than three G and C nucleotides. The collection of oligonucleotide probes has oligonucleotides having a melting temperature in ° C. less than 12 times the number of tetramers and more than 18 times the number of tetramers. This aspect of the present invention also includes reporter labels linked to each of the oligonucleotide probes in the collection.

Designing a Plurality of Translational Oligonucleotides for Attachment to Target-Specific Oligonucleotide Probes

The present invention also relates to a method of designing a plurality of translational oligonucleotides for attachment to target-specific oligonucleotide probes to identify or quantify complementary sequences which will hybridize with little mismatch, where the plural translating oligonucleotide sequences have melting temperatures within a narrow range. This involves providing a first set of a plurality of tetramers of four nucleotides linked together, where (1) each tetramer within the set differs from all other tetramers in the set by at least two nucleotide bases, (2) no two tetramers within a set are complementary to one another, (3) no tetramers within a set are palindromic or dinucleotide repeats, and (4) no tetramer within a set has less than one or more than three G and C nucleotides. Groups of 2 to 4 tetramers from the first set are linked together to form a collection of multimer units. All multimer units formed from the same tetramer and all multimer units having a melting temperature in ° C. of less than 3 times the number of tetramers forming a multimer unit are removed from the collection of multimer units to form a modified collection of multimer units. The modified collection of multimer units are arranged in a list in order of melting temperature. The order of the modified collection of multimer units is randomized in 0.1° C. increments of melting temperature. Alternating multimer units in the list are divided into first and second subcollections, each arranged in order of melting temperature. The order of the second subcollection is inverted. In order, the first subcollection of multimer units is linked to the inverted second subcollection of multimer units in order to form a collection of double multimer units. The collection of double multimer units is arranged in a list in order of melting temperature. Those units having a melting temperature in ° C. of less than 12 times the number of tetramers and more than 18 times the number of tetramers are removed from the ordered collection of double multimer units to form a modified collection of multimer units. The double multimer units are linked to a target-specific oligonucleotide probe.

The present invention also relates to a collection of fusion oligonucleotide probes. These probes includes a collection of translational oligonucleotide probes to which complementary oligonucleotide probes will hybridize, within a narrow temperature range of greater than 24° C. with little mismatch. The oligonucleotide probes are formed from sets of tetramers where (1) each tetramer within the set differs from all other tetramers in the set by at least two nucleotide bases, (2) no two tetramers within a set are complementary to one another, and (3) no tetramers within a set are palindromic or dinucleotide repeats, and (4) no tetramer within a set has less than one or more than three G and C nucleotides. The collection of oligonucleotide probes has oligonucleotides having a melting temperature in ° C. less than 12 times the number of tetramers and more than 18 times the number of tetramers. Target-specific oligonucleotide probes linked to each of the oligonucleotide probes in the collection.

Mini-Cycling

When a LDR is performed on two different targets, the percent yield from each will depend upon the abilities of the different probes to ligate at each target region. For example, if probes at Gene 1 ligate with an efficiency of 40%, and at Gene 2 with an efficiency of 80%, analysis of the results would falsely indicate that Gene 2 copy number is twice that of target 1. Differences in ligation efficiency have been adjusted for by always comparing results from the tumor sample with the normal control. In the example above, if the product yield for Control Gene 1=400, Cancer Gene 2=800 in the tumor, and Control Gene 1=300, Cancer Gene 2=600 in the normal sample, the ratio of ratios=800/600:400/300=1.33:1.33=1:1, indicating no amplification of Cancer Gene 2 in the tumor. If product yield for Control Gene 1=400, Cancer Gene 2=800 in the tumor, and Control Gene 1=300, Cancer Gene 2=200 in the normal sample, the ratio of ratios=800/200: 400/300=4:1.33=3:1, or three-fold amplification of Cancer Gene 2 in the tumor.

However, there are situations where a direct comparison of copy number from different gene targets would be preferred. This may be achieved by driving the ligation on a target to completion.

The basic format of an LDR is to denature the DNA, allow annealing of probes, ligation, and repeating several times, giving linear amplification. With mini-cycling, once ligation has occurred, the temperature is increased to dissociate unligated probes, but the temperature is not increased sufficiently to cause the ligation product to dissociate. The temperature is then allowed to fall so that annealing and ligation are repeated, thus increasing the amount of ligation product that is annealed to the target region. For example, ligation may be performed at 65° C. using probes with Tₘ values of 65° C., and then the temperature may be raised to 75° C. to dissociate unligated probes, but not ligation product. In these examples, the mini-cycling is repeated up to 10 times. Four different situations are considered below:

a) Equalizing Ligation Yields on Two Different Targets:
P₍ᵢ₎=x(100−P₍ᵢ₋₁₎)+P₍ᵢ₋₁₎
Where P₍ᵢ₎=the product yield for a given target after i mini-cycles and x=the efficiency of ligation.

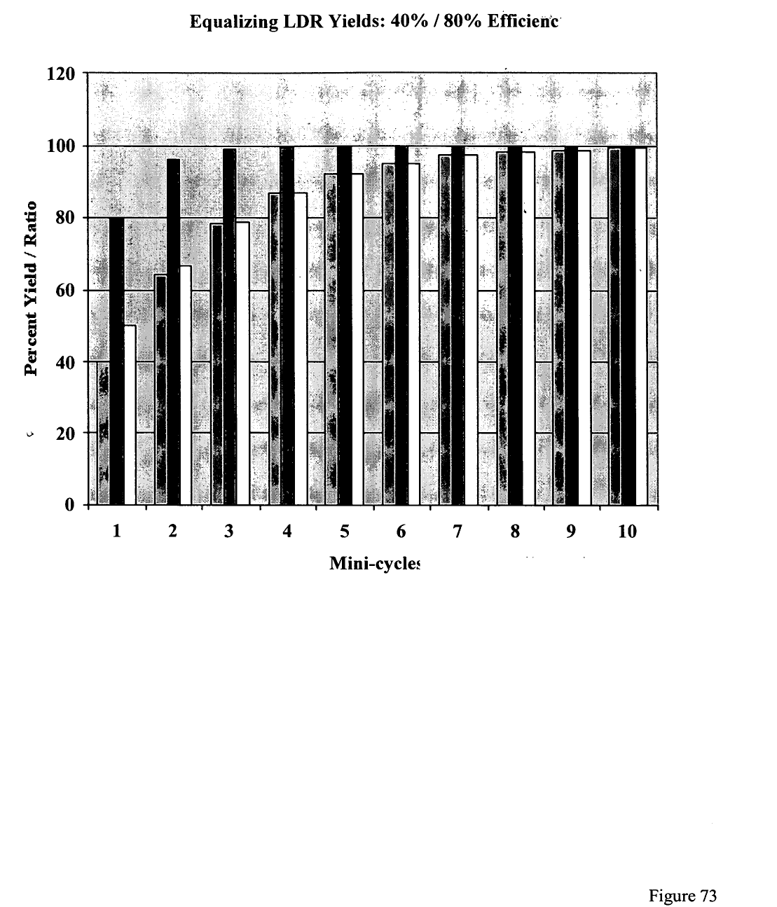

FIG. 73 considers a situation where probes ligate at target 1 with an efficiency of 40%. This leaves 60% of the target available for ligation in the next round of mini-cycling. Again, probes ligate at the remaining 60% of this target with an efficiency of 40%, so in this round, a further 24% of the target is covered. This leaves 36% of the target available for ligation in the next mini-cycle, etc. In this way, 99% of the target region has ligation product molecules annealed after 10 rounds of mini-cycling. For target 2, probes ligate with an efficiency of 80%, so after the initial annealing and ligation, there is twice as much ligation product from this target compared to target 1. After 10 mini-cycles, 100% of the target region has ligation product molecules attached. Thus, 10 rounds of mini-cycling has virtually eliminated any bias in the ratio of products from the two targets.

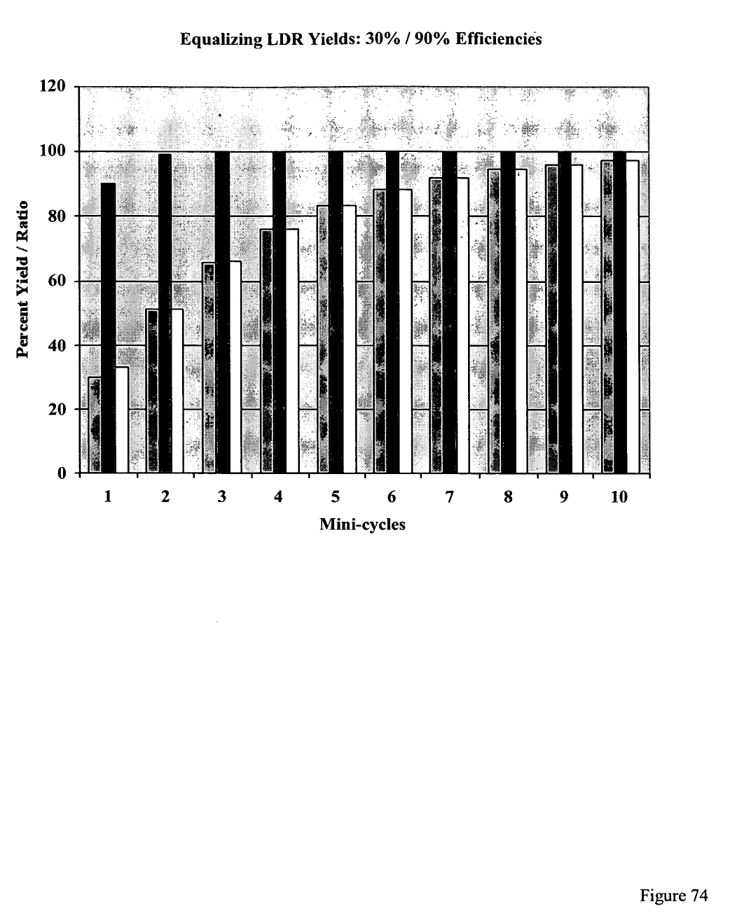

In FIG. 74, a situation is considered where the efficiency of probe ligation at target 1 is one third that of target 2. Following 10 rounds of mini-cycling, 97% of target I has ligation product molecules annealed, compared to 100% of target 2, so it is evident that the initial 1:3 ratio of product yields has again been virtually eliminated.
(b) Equalizing Ligation Yields of Two Different SNPs on the Same Target:

$${P}_{( i )} = \frac{x}{2} ( 100 - {P}_{( i - 1 )} ) + {P}_{( i - 1 )}$$

Where P₍ᵢ₎=the product yield for a given target after i mini-cycles and x=the efficiency of ligation.

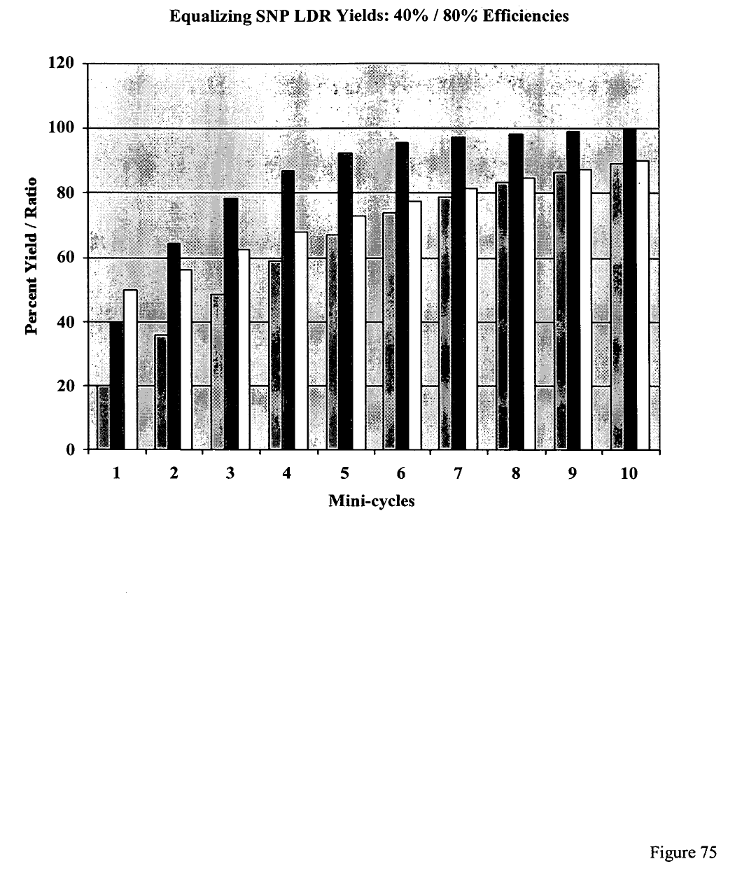

In the case of single nucleotide polymorphisms (SNPs), probes designed to distinguish between wild-type and mutant alleles will differ only at a single position. Therefore, both probes will anneal at a particular target, but only the perfectly matched probe at the junction will allow ligation to occur. In FIG. 75, the two discriminating probes compete for ligation at each target. Therefore, the efficiency with which ligation will occur is halved at each probe. In this example, the efficiency for probe 1 is 40%, and for probe 2 80%, but since they compete for the same target, the yields are 20% and 40%, respectively. After the initial ligation, it can be seen that the percent yield from probe 2 is twice that from probe 1. However, following ten rounds of mini-cycling, the percent yield from probe 1 is 89%, and from probe 2 99%, and thus one ligation yield is within 90% of the other.

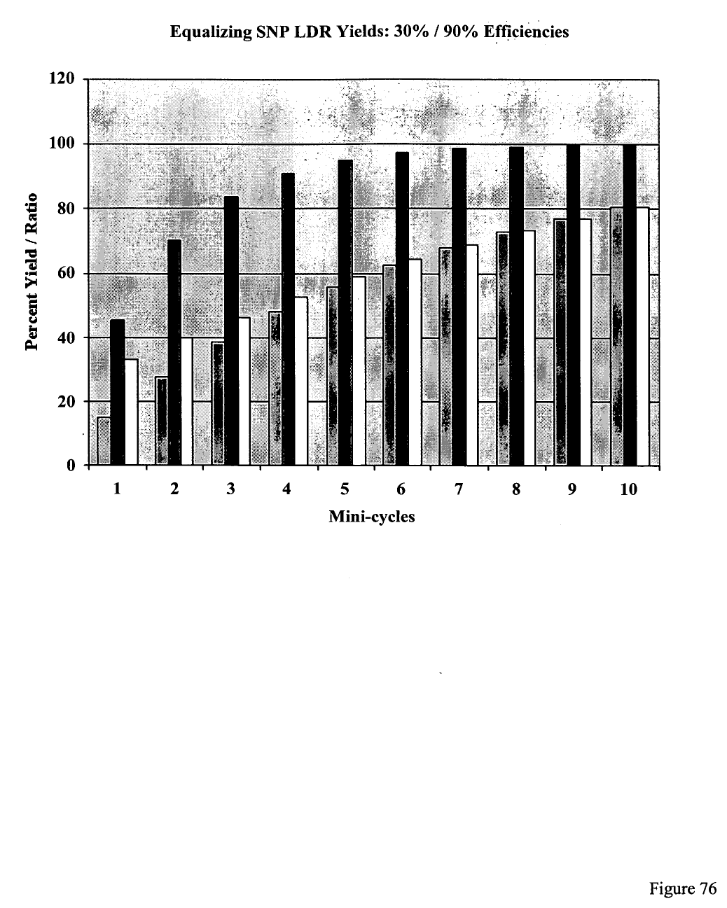

In FIG. 76, target regions with ligation efficiencies of 30% and 90% are examined, but again, for each SNP there are two competing probes, reducing the effective efficiency of ligation to 15% and 45% respectively. Following the first ligation, the percent yield from target 1 is thus one third that of target 1. After 10 rounds of mini-cycling, however, this bias is reduced to 80%.
(c) Equalizing Ligation Yields on Two Different Targets, with 5% Product Dissociation:

$${P}_{( i )} = x ( 100 - 0.95 {P}_{( i - 1 )} ) + 0.95 {P}_{( i - 1 )} + {\sum }_{1}^{i - 1} 0.05 P$$

Where P₍ᵢ₎=the product yield for a given target after i mini-cycles and x=the efficiency of ligation.

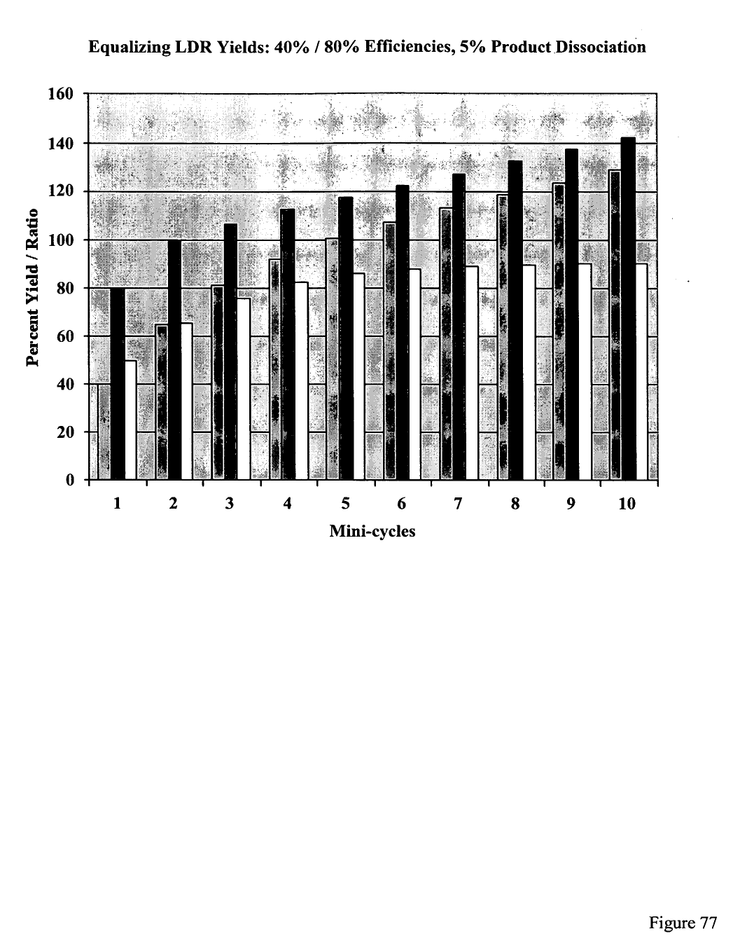

The mini-cycling temperatures may require some optimization. Under conditions that allow efficient dissociation of unligated probes, a small fraction of ligation products may dissociate from the target regions. It is estimated that less than 5% of the ligation product may dissociate during each round of mini-cycling. In the example, probes ligate at target 1 with an efficiency of 40%, in the first round of mini-cycling. Although 40% of the available target will be represented in the ligation product, 5% of this product will dissociate from the target, and so more target will become exposed for annealing of probes in the next round of mini-cycling. Since a proportion of the target region becomes available for probe annealing again due to product dissociation, the percent yield from each target region can exceed 100%. In FIG. 77, from a starting efficiency of 40%, after 10 rounds of mini-cycling, the percent yield from target 1 is 128%, and from target 2, with a probe binding efficiency of 80%, the yield is 142%. The ratio of target 1 to target 2 has improved from 50% to 90%.

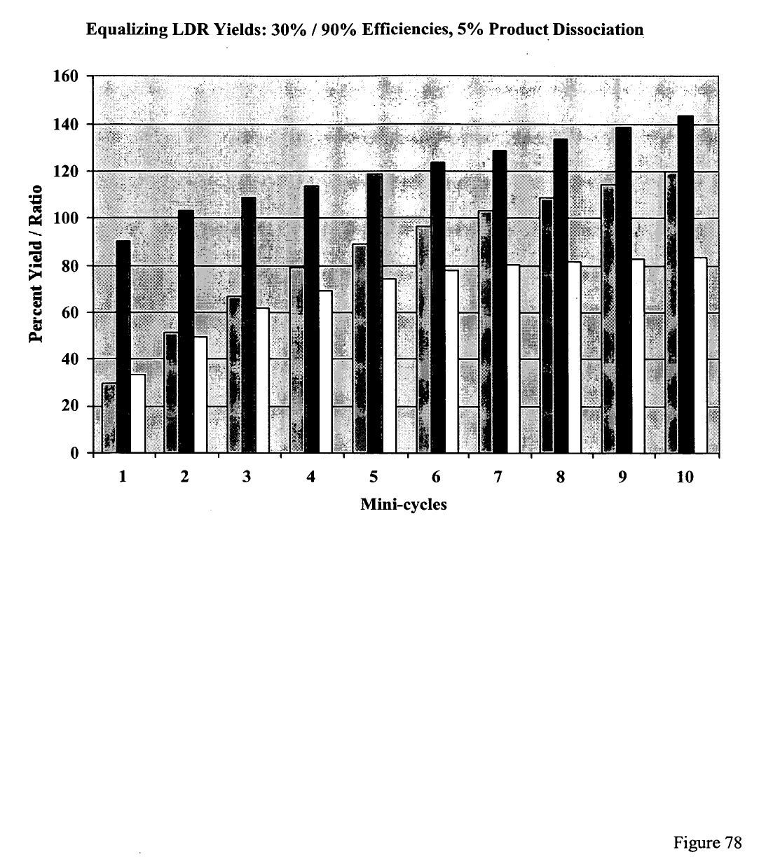

In FIG. 78, from a starting efficiency of 30%, after 10 rounds of mini-cycling, the percent yield from target 1 is 120% and from target 2, with a ligation efficiency of 90%, the percent yield is 144%, so the ratio has improved from 33% to 83%.
(d) Equalizing Ligation Yields of Two Different SNPs on the Same Target, with 5% Product Dissociation:

$${P}_{( i )} = \frac{x}{2} ( 100 - 0.95 {P}_{( i - 1 )} ) + 0.95 {P}_{( i - 1 )} + {\sum }_{1}^{i - 1} 0.05 P$$

Where P₍ᵢ₎=the product yield for a given target after i mini-cycles and x=the efficiency of ligation.

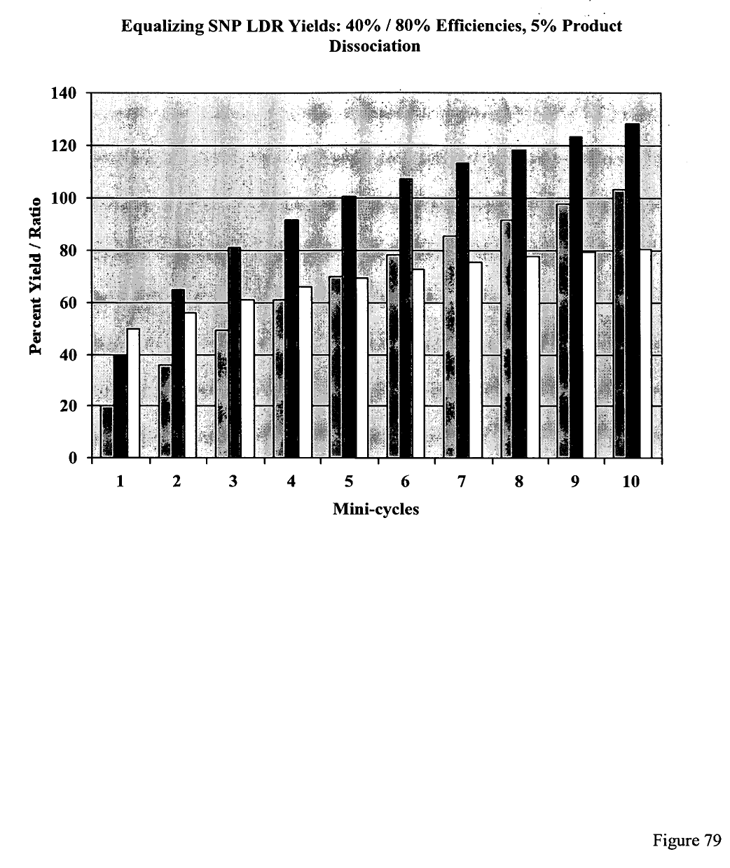

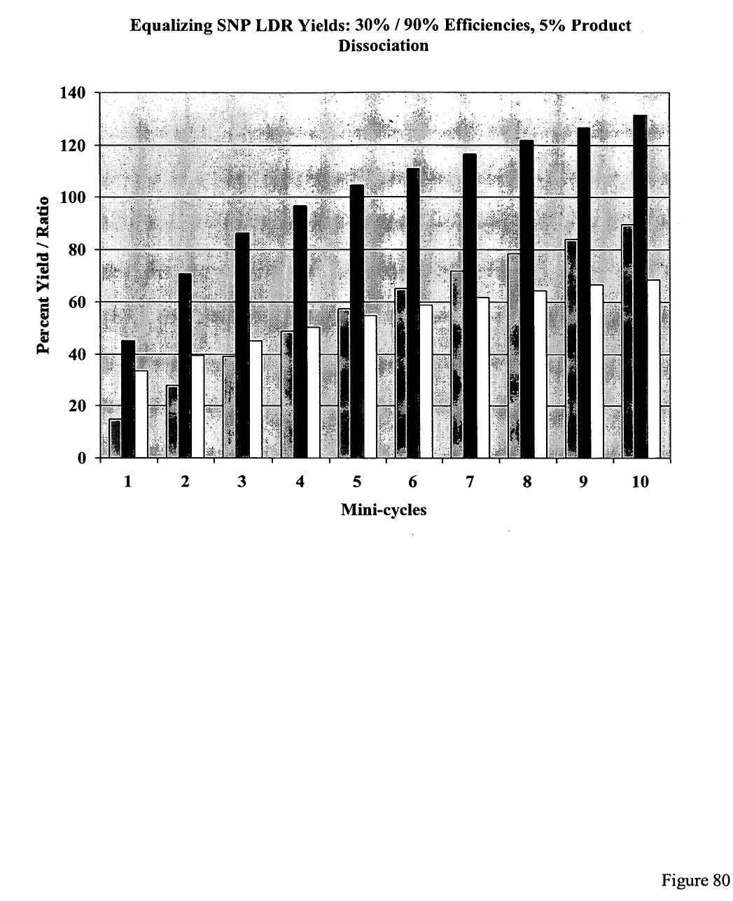

In FIGS. 79-80, a situation where there are two competing probes for each target region, combined with 5% dissociation of ligation product is examined. In FIG. 79, the efficiency of probes ligation at target 1 and 2 are 40% and 80%, respectively, but because of competition between probes, the overall respective efficiencies are 20% and 40%. Following ten rounds of mini-cycling, the percent yield from target 1 is 103% and from target 2 128%, so the ratio of percent yields has risen from 50% to 81% with mini-cycling.

In FIG. 80, the efficiency of ligation to targets 1 and 2 are 30% and 90%, respectively, but, as seen above, the overall efficiencies of probe binding are 15% and 45%, respectively, due to competition between probes. Following ten rounds of mini-cycling, the percent yield from target 1 is 90%, and from target 2 is 131%. Thus, the ratio of percent yields has increased from 33% in the first round to 68% after ten rounds of mini-cycling.

In summary, minicycling ligation reactions contain internal cycles comprising of a hybridization treatment, wherein the oligonucleotide probe sets hybridize at adjacent positions in a base-specific manner to their respective target nucleotide sequences, if present in the sample, and ligate to one another to form a ligation product, and a probe denaturation treatment, where the reaction is heated to above the melting temperature of each probe-specific portion so unligated probes separate from the target, but below the melting temperature of each product-specific portion such that ligation products accumulate on the target with each successive internal cycle (i.e. minicycle), to provide a more quantitative measure of the relative level of two or more target nucleotide sequences in the sample.

### EXAMPLES

### Example 1

### Reagents, Equipment, and Oligodeoxynucleotides

All routine chemical reagents were purchased from Sigma Chemicals (St. Louis, Mo.) or Fisher Scientific (Fair Lawn, N.J.). Microcon YM-50 spin columns were purchased from Millipore Corporation (Bedford, Mass.). Thin-walled 250 μl PCR-tubes were purchased from the Applied Biosystems Division of Perkin-Elmer corporation (Foster City, Calif.).

Streptavidin-coated Proactive magnetic microspheres, with a mean diameter of 860 nm and binding capacity of 686 pmol biotin-FITC/mg., were purchased from Bangs Laboratories, Inc. (Fishers, Ind.). Streptavidin-coated quantum dots, with an emission maximum of 608-611 nm and carrying 3 to 7 streptavidin molecules per Q-dot, were purchased from Quantum Dot Corporation (Hayward, Calif.).

Hybridizations and incubations were performed in a LabLine 307 Hybridization Incubator (Melrose Park, Ill.). Magnetic capture of microspheres was done using a nickel-alloy magnet supplied by Clemente Associates, Inc. (Madison, Conn.). Fluorescence was detected using a GSI Lumonics ScanArray 5000 (Billerica, Mass.) and was quantified by the Packard Biochip Technologies' ScanArray Microarray Acquisition Software (Billerica, Mass.). The absorbance and emission maxima for various fluorescent and near infrared dyes is listed in Table 1, below.

TABLE 1
Absorbance and emission maxima for the various
fluorescent and near infrared dyes

| Dye | Abs. Max(nm) | Em. Max (nm) |
|---|---|---|
| Marina Blue | 365 | 460 |
| Fluorescein | 495 | 520 |
| TET | 521 | 536 |
| TAMRA | 565 | 580 |
| Rhodamine | 575 | 590 |
| ROX | 585 | 610 |
| Texas Red | 600 | 615 |
| Cy2 | 489 | 506 |
| Cy3 | 550 | 570 |
| Cy3.5 | 581 | 596 |
| Cy5 | 649 | 670 |
| Cy5.5 | 675 | 694 |
| Cy7 | 743 | 767 |
| Spectrum Aqua | 433 | 480 |
| Spectrum Green | 509 | 538 |
| Spectrum Orange | 559 | 588 |
| BODIPY FL | 505 | 515 |
| BODIPY R6G | 530 | 550 |
| BODIPY TMR | 545 | 575 |
| BODIPY 564/6570 | 565 | 575 |
| BODIPY 581/591 | 580 | 600 |
| BODIPY TR | 595 | 625 |
| BODIPY 630/650 | 640 | 650 |
| ABI, 5-FAM | 494 | 522 |
| ABI, JOE | 520 | 548 |
| ABI, TAMRA | 560 | 582 |
| ABI, ROX | 588 | 608 |
| YO-PRO ™ -3 | 612 | 631 |
| YOYO ™ -3 | 612 | 631 |
| R-phycocyanin | 618 | 642 |
| C-Phycocyanin | 620 | 648 |
| RiboGreen ™ | 500 | 525 |
| Rhodamine Green | 502 | 527 |
| Rhodamine 123 | 507 | 529 |
| Magnesium Green ™ | 506 | 531 |
| Calcium Green ™ | 506 | 533 |
| TO-PRO ™ -1 | 514 | 533 |
| TOTO ® -1 | 514 | 533 |
| YO-PRO ™ -1 | 491 | 509 |
| YOYO ™ -1 | 491 | 509 |
| Alexa ™ 488 | 490 | 520 |
| Alexa ™ 546 | 555 | 570 |
| Alexa ™ 594 | 590 | 615 |
| Texas Red ® | 595 | 615 |
| Nile Red | 549 | 628 |
| Magnesium Orange ™ | 550 | 575 |
| Phycoerythrin, R & B | 565 | 575 |
| Rhodamine Phalloidin | 550 | 575 |
| Calcium Orange ™ | 549 | 576 |
| Pyronin Y | 555 | 580 |
| Rhodamine B | 555 | 580 |
| DiD DilC(5) | 644 | 665 |
| Thiadicarbocyanine | 651 | 671 |
| FAR-Green One (SE) | 800 | 820 |
| FAR-Green Two (SE) | 772 | 788 |
| DY-750 | 747 | 776 |
| DY-782 | 782 | 800 |
| Alexa Fluor 750 | 749 | 775 |

Oligonucleotides (Table 2) were purchased from Integrated DNA Technologies, Inc. (Coralville, Iowa) or were synthesised on an Expedite Multiple Oligo Synthesis System (PerSeptive Biosystems, Framingham, Mass., a part of the Applied Biosystems family of companies) using reagents supplied by Applied Biosystems.

TABLE 2
Oligonucleotides

| | |
|---|---|
| Oligonucleotide name | Oligonucleotide sequence |

| | | |
|---|---|---|
| BcQz122 | 5′Biotin TEG-Spacer18- | |
| | TTGAAATCCAGCGCAAAATCTGCGAAGCCGTGCGCAACGACGCA | (SEQ ID NO: 38889) |
| | TTGCGGCTTTCGCGCATTGCGGCTTTCG 3′ | |
| Q-Zip1 | 5′ Biotin TEG-Biotin TEG-Spacer 18- | |
| | TCGTTGCGCACGGC(UOMe)(UOMe) 3′ | (SEQ ID NO: 38890) |
| Q-Zip2 | 5′ Biotin TEG-Biotin TEG-Spacer18- | |
| | CGAAAGCCGCAATG(COMe)(GOMe) 3′ | (SEQ ID NO: 38891) |
| Q-Zip4 | 5′ Biotin TEG-Biotin TEG-Spacer18- | |
| | TGCGCTTGCAGCCG(UOMe)(UOMe) 3′ | (SEQ ID NO: 38892) |
| Cy3Qz1 | 5′ Cy3-TCGTTGCGCACGGCTT 3′ | (SEQ ID NO: 38893) |
| Cy3p16Ex1 | 5′ Cy3-ATTGGTTTTTGATTGTAATTATTTGGTGC 3′ | (SEQ ID NO: 38894) |
| Zip1 | 5′ TTGAAATCCAGCGCAAAATCTGCGAAGCC-Spacer18- | (SEQ ID NO: 38895) |
| | Biotin TEG 3′ | |
| cZ1cQ1cQ2 | 5′ CGCAGATTTTGCGCTGGATTTCAATGTGGTAGTTGGAGCT | (SEQ ID NO: 38896) |
| | GGTG AAGCCGTGCGCAACGACGCATTGCGGCTTTCG 3′ | |

### Example 2

### Selection of a Set of 16 Q-Zip Sequences of 16 Bases Each with High Tₘ Values that are within a Close Range of Each Other (65° C. to 68° C.)

Each Q-zip-code sequence is composed of four tetramers (designed as described in the following) so that the full length 16-mers have similar Tₘ values. The 256 (44) possible combinations in which four bases can be arranged as tetramers were reduced to a set of 36; these were chosen so that each tetramer differs from all others by at least two bases (See Table 3). Tetramer complements, as well as tetramers that would result in self-pairing or hairpin formation of the zip codes, were eliminated. Additionally, tetramers that were palindromic, e.g., TCGA, or repetitive, e.g., CACA, were excluded. The indicated set of thirty-six tetramers represents just one of the possible sets that can be created. In the following description, a tetramer is referred to by its designation in the first column of Table 3. For example, #29=TGCG.

TABLE 3
Original Tetramers used to construct Universal
Arrays, Universal Beads, and Q-zips

| | Original | Tetramer | Tetramer | |
|---|---|---|---|---|
| New tetramer | tetramer | sequence | complement | G + C |
| designation | designation | 5′-3′ | 5′-3′ | bases |
| 1 | 6 | TTGA | TCAA | 1 |
| 2 | 7 | TGAT | ATCA | 1 |
| 3 | 8 | TTAG | CTAA | 1 |
| 4 | 26 | AATC | GATT | 1 |
| 5 | 31 | ATAC | GTAT | 1 |
| 6 | 32 | AAAG | CTTT | 1 |
| 7 | 36 | TACA | TGTA | 1 |
| 8 | 1 | TCTG | CAGA | 2 |
| 9 | 2 | TGTC | GACA | 2 |
| 10 | 5 | TCGT | ACGA | 2 |
| 11 | 9 | CTTG | CAAG | 2 |
| 12 | 10 | CGTT | AACG | 2 |
| 13 | 11 | CTCA | TGAG | 2 |
| 14 | 13 | CTGT | ACAG | 2 |
| 15 | 15 | CCAT | ATGG | 2 |
| 16 | 16 | CGAA | TTCG | 2 |
| 17 | 17 | GCTT | AAGC | 2 |
| 18 | 18 | GGTA | TACC | 2 |
| 19 | 19 | GTCT | AGAC | 2 |
| 20 | 21 | GAGT | ACTC | 2 |
| 21 | 23 | GCAA | TTGC | 2 |
| 22 | 25 | AGTG | CACT | 2 |
| 23 | 27 | ACCT | AGGT | 2 |
| 24 | 28 | ATCG | CGAT | 2 |
| 25 | 30 | AGGA | TCCT | 2 |
| 26 | 33 | CCTA | TAGG | 2 |
| 27 | 34 | GATG | CATC | 2 |
| 28 | 3 | TCCC | GGGA | 3 |
| 29 | 4 | TGCG | CGCA | 3 |
| 30 | 12 | CACG | CGTG | 3 |
| 31 | 14 | CAGC | GCTG | 3 |
| 32 | 20 | GACC | GGTC | 3 |
| 33 | 22 | GTGC | GCAC | 3 |
| 34 | 24 | GGAC | GTCC | 3 |
| 35 | 29 | ACGG | CCGT | 3 |
| 36 | 35 | AGCC | GGCT | 3 |

This procedure involved the following steps:

1. Create two columns, 1-46656 all variations of 36×36×36 tetramers.

2. Compute Tₘ values of 46656 12-mers in Oligo 6.4 and sort according to T_{m.}

3. Remove trimer-tetramers (designated herein as “trimers” i.e. 3 tetramers together yielding a 12mer) that contain 1 GC base in each tetramer or contain 3 GC bases in each tetramer.

4. Select trimers (i.e. 12mers) with Tm values between 48 and 50.5. There are 410 “trimer-tetramers” in this list.

5. Since #29 appears in many of the “410 list,” the following procedure is suggested to save #29 for trimers where it is really needed. Remove all trimers with #29 in them.

6. Start selecting some trimers from the remaining list. These are placed onto short list (Short List 1 Tm 48-50.5). For each trimer selected, remove the pair from the No #29 trimers list. For example, if a trimer uses tetramers #28, #34, and #17 in that order, then other trimers containing #28 followed by #34, or #34 followed by #17 should be removed from the list.

7. Create pairs used list to avoid using the same pair of tetramers twice. (Pairs used from Tm 48-50.5).

8. When a tetramer has been used twice in a given column, it should be “retired” and preferably not used again.

9. Now go back to “410 list” and use trimers with 29. Short list now stands at 12 trimers=28 pairs used.

10. Remove pairs from 410 list.

11. Select four more trimers. Put onto short list (Short List 1 Tm 48-50.5). For each trimer selected, remove the pair from the 410 trimers list. The tetramer 29 was used three times in the last position.

12. With a total of 16 probes of length 12, with calculated Tₘ values of 48-49.2, go to 37787 trimers and look up trimers with the first two tetramers the same as the last two tetramers of the Short list primers. Arrange these in order of ascending Tₘ value, and write down the last several tetramers for that position with the caveat that (i) it cannot be the same as one of the existing tetramers in the short list probes, and (ii) it cannot yield a pair which has already been used.

13. Manually choose the last tetramer and add to the short list. (Short List 3+1 Tm 48-50.5).

14. Add newly formed pair to “Pairs used from Tm 48-50.5” list, and verify that no pair has been used twice.

15. Copy sequences into Oligo 6.4. Determine Tₘ of each 16 mer. Range from 64.1 to 68. (Results Short list 3+1.1 Tm)

16. Take 4 lowest sequences and 3 highest sequences and rearrange last tetramers to try to increase lower Tm values, and decrease higher ones. (Short List 3+1.2 W Tm 64.1-68).

17. Copy sequences into Oligo 6.4. Determine Tm of each 16 mer. Range from 64.1 to 68. (Results Short list 3+1.2 W Tm).

18. All sequences moved toward the means except one increased slightly. Adjust manually again.

19. Take final sequence into a new document (Short List 3+1.2 Tm 65.5-67.4), and verify Tms with Oligo 6.4 (Results Short list 3+1.2 Tm), verify pairs (Pairs used from Tm 65.5-67.4).

20. For some applications, Q-Zips and zip-codes will be used together. Therefore, it would be preferable that the two do not have any identical sequences of 16 contiguous bases. Go to master list of 4633 universal zip codes and check to see if Q-Zip tetramers (i.e. 16mers) are used in hexamer (i.e. 24mer) sequences. One sequence was found with a match=Zip code #1459=tetramer #2, #27, #33, #17, #29, #12 with QZ12=#33, #17, #29, #12=GTGCGCTTTGCGCGTT (SEQ ID NO: 38883).

21. Change QZ12 into #33, #17, #29, #31=GTGCGCTTTGCGCAGC (SEQ ID NO: 38884). Incorporate change into other documents.

22. Analyze Q-zipcode sequences for hairpin structures. QZ6=TGCGCTTGCAGCGCTT (SEQ ID NO: 38885) has a 5 bp hairpin. Also, QZ12=GTGCGCTTTGCGCAGC (SEQ ID NO: 38886) has a 5 bp hairpin.

23. Fix QZ6=TGCGCTTGCAGCCGTT (SEQ ID NO: 6) reduce hairpin to 3. Fix QZ12=GTGCGCTTTGCGACGG (SEQ ID NO:12), reduce hairpin to 3.

24. Go back to master list of 4633 zip codes and check to see if replacement Q-Zip tetramers (i.e. 16mer) are used in universal zip-code hexamer (i.e. 24mer) sequences. Now, none are used. Incorporate change into other documents.

25. Final list of Q-zip-code sequences and complements are provided in Table 4.

TABLE 4
Q-dot zip-code sequences and their components.

| | | | | | |
|---|---|---|---|---|---|
| | | | | Tetramer | |
| Q-dot Zip | Tri-# | Tm | Sequence (5′-3′) | Designations | Complement (5′-3′) |

| | | | | | | | | |
|---|---|---|---|---|---|---|---|---|
| QdotZ1 | 12702 | 66.6 | TCGTTGCGCACGGCTT | 10 | 29 | 30 | 17 | AAGCCGTGCGCAACGA |
| | | | (SEQ ID NO:1) | | | | | (SEQ ID NO:17) |
| QdotZ2 | 20721 | 66.9 | CGAAAGCCGCAATGCG | 16 | 36 | 21 | 29 | CGCATTGCGGCTTTCG |
| | | | (SEQ ID NO:2) | | | | | (SEQ ID NO:18) |
| QdotZ3 | 36197 | 66.2 | TCCCGGACGCTTGCAA | 28 | 34 | 17 | 21 | TTGCAAGCGTCCGGGA |
| | | | (SEQ ID NO:3) | | | | | (SEQ ID NO:19) |
| QdotZ4 | 36679 | 66.8 | TGCGCTTGCAGCCGTT | 29 | 11 | 31 | 12 | AACGGCTGCAAGCGCA |
| | | | (SEQ ID NO:4) | | | | | (SEQ ID NO:20) |
| QdotZ5 | 38757 | 66.4 | CACGGTGCGCAAAGCC | 30 | 33 | 21 | 36 | GGCTTTGCGCACCGTG |
| | | | (SEQ ID NO:5) | | | | | (SEQ ID NO:21) |
| QdotZ6 | 40061 | 66.9 | CAGCGTGCTGCGCCAT | 31 | 33 | 29 | 15 | ATGGCGCAGCACGCTG |
| | | | (SEQ ID NO:6) | | | | | (SEQ ID NO:22) |
| QdotZ7 | 44488 | 67 | ACGGCGTTTCCCCGAA | 35 | 12 | 28 | 16 | TTCGGGGAAACGCCGT |
| | | | (SEQ ID NO:7) | | | | | (SEQ ID NO:23) |
| QdotZ8 | 46456 | 66.7 | AGCCCAGCCGAAACGG | 36 | 31 | 16 | 35 | CCGTTTCGGCTGGGCT |
| | | | (SEQ ID NO:8) | | | | | (SEQ ID NO:24) |
| QdotZ9 | 37032 | 66.7 | TGCGGCAAATCGCGTT | 29 | 21 | 24 | 12 | AACGCGATTTGCCGCA |
| | | | (SEQ ID NO:9) | | | | | (SEQ ID NO:25) |
| QdotZ10 | 39635 | 67.4 | CAGCGCAAACGGCGAA | 31 | 21 | 35 | 16 | TTCGCCGTTTGCGCTG |
| | | | (SEQ ID NO:10) | | | | | (SEQ ID NO:26) |
| QdotZ11 | 42077 | 66.6 | GTGCGCTTTGCGACGG | 33 | 17 | 29 | 35 | CCGTCGCAAAGCGCAC |
| | | | (SEQ ID NO:11) | | | | | (SEQ ID NO:27) |
| QdotZ12 | 45213 | 67 | ACGGGACCGTGCAGCC | 35 | 32 | 33 | 36 | GGCTGCACGGTCCCGT |
| | | | (SEQ ID NO:12) | | | | | (SEQ ID NO:28) |
| QdotZ13 | 15280 | 65.5 | CGTTTGCGCGAATCCC | 12 | 29 | 16 | 28 | GGGATTCGCGCAAACG |
| | | | (SEQ ID NO:13) | | | | | (SEQ ID NO:29) |
| QdotZ14 | 36051 | 66.7 | TCCCCACGCCATGCAA | 28 | 30 | 15 | 21 | TTGCATGGCGTGGGGA |
| | | | (SEQ ID NO:14) | | | | | (SEQ ID NO:30) |
| QdotZ15 | 38693 | 67.4 | CACGCAGCTGCGGTGC | 30 | 31 | 29 | 33 | GCACCGCAGCTGCGTG |
| | | | (SEQ ID NO:15) | | | | | (SEQ ID NO:31) |
| QdotZ16 | 45964 | 66.9 | AGCCGCTTTCCCGTGC | 36 | 17 | 28 | 33 | GCACGGGAAAGCGGCT |
| | | | (SEQ ID NO:16) | | | | | (SEQ ID NO:32) |

26. Suitable Q-dot-zip codes according to the present invention include, without limitation, those shown in FIG. 96. FIG. 96 shows a list of 16 Q-dot-zip codes (SEQ ID NO:1 through SEQ ID NO:16), and their 16 complements (SEQ ID NO:17 through SEQ ID NO:32). The Q-Zips are each 16mer.

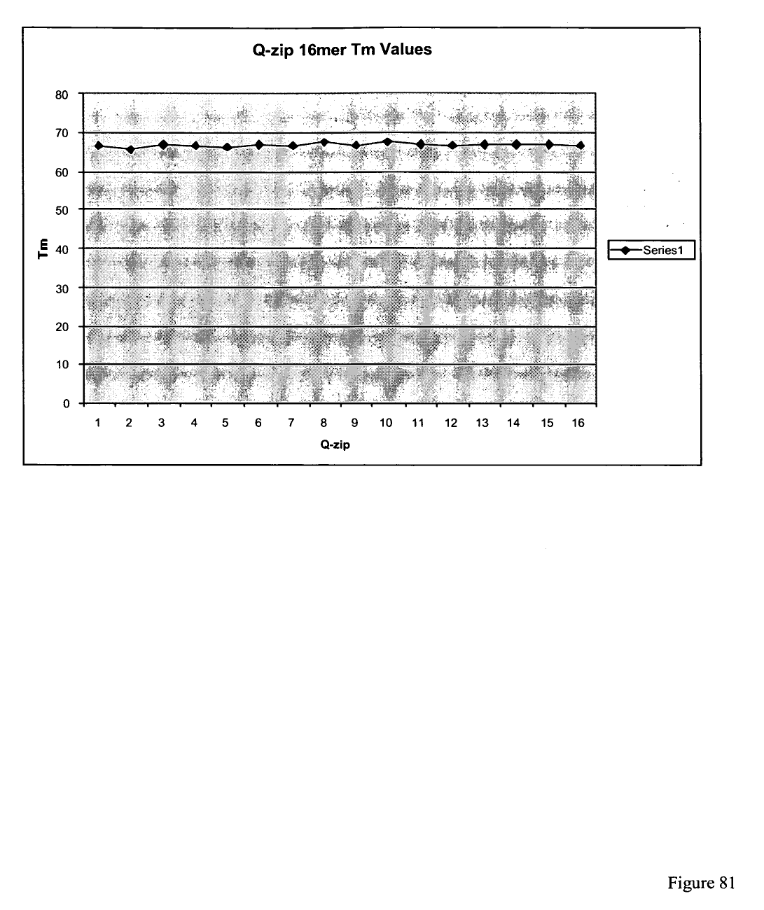

27. FIG. 81 is a graph showing the Tm values of Q-Zip codes. Q-Zip codes, 16mers with Tm values from 63° C. to 69.5° C., are attached to Q-dots to aid in encrypting signal. See FIG. 96.

### Example 3

### Construction of Q-Zip Tails with Three or Four Q-dots Together to Score any LDR Product

Q-Zip tails can be constructed in the following ways:

1. Take list of Q-Zips from “Q-Zip List Trimer Construction” document. Reorder them to have first group of 8 QDot-zips, next 4 QDot-zips (12 total), and last 4 QDot-zips (16 total).

2. Determine Complements of New QdotZ1-Z16 (Q-dot zip+Complement Tm65.5-67.4). Final list of Q-zip-code sequences and complements are provided in Table 3.

3. For the following descriptions and numbering system, each Q-Zip is now designated by its Q-object number. For example, Q# 2=5′ CGAAAGCCGCAATGCG 3′ (SEQ ID NO: 2) (see Table 3).

4. Make a list of all combinations of 12 things taken 3 at a time, with no repeats. To avoid repeats each subsequent column has to have an object number greater than the previous. Total=12×11×10/1×2×3=220.

5. Relabel this list and provide numbers 1-220.

6. Expand the list to include all combinations of 12 things taken 4 at a time, with no repeats. Total=12×11×10×9/1×2×3×4=495. Provide numbers 1-495.

7. Take list of 12 things taken 3 at a time, with no repeats, copy three times, moving the second and the third column to the front for each copy. Recopy that column to obtain all the ways you can have 12 things, 4 at a time, 1st number repeated twice, the other two different. Total=3×12×11×10/1×2×3=3×220=660 Reorder these and number 1-660.

8. Add the repeats to the bottom of the unique 4 at a time list=1155 objects.

9. Reduce this set to also list unique and repeats for 8-Q-dots=8×7×6×5/1×2×3×4=70; add 3×8×7×6/1×2×3=3×56=168. Total=238.

10. Expand the above set to also list unique and repeats for 16-Q-dots=16×15×14×13/1×2×3×4=1820; add 3×16×15×14/1×2×3=3×560=1680. Total=3500.

11. The above results suggest a substantial number of Q-zip tails may be available from just three 16mers. The design of tails should start with a limited number of Q-dots, and expand to include an increasingly larger set. Further, the oligos should be randomized within the initial set to provide a balance in QDot-zip usage. Therefore, prepare sets of three 16mers (48mer) starting with 4, 8, 12, and 16 Q-dot-Zips.

12. Four (4) things taken 3 at a time, with no repeats. Total=4×3×2/1×2×3=4. Then add 4 things, three at a time with 1st number repeated twice=2×4×3/2×1=12. Total=16.

13. Eight (8) things taken 3 at a time, with no repeats. Total=8×7×6/1×2×3=56. Then add 8 things, three at a time with 1st number repeated twice=2×8×7/2×1=56. Total=112. 14. Twelve (12) things taken 3 at a time, with no repeats. Total=12×11×10/1×2×3=220. Then add 12 things, three at a time with 1st number repeated twice=2×12×11/2×1=132. Total=352.

15. Sixteen (16) things taken 3 at a time, with no repeats. Total=16×15×14/1×2×3=560. Then add 12 things, three at a time with 1st number repeated twice=2×16×15/2×1=240. Total=800.

16. The list of Q-Zip tails needs to be randomized, while still retaining the general order of 4 to 16 Q-dots. In other words, if only 8 Q-dots are available at a given time, then those tails need to be used first. Also, if for some technical reason the repeat of a Q-dot provides technical difficulties (i.e. it turns out that some Q-dots have two primers attached, both of which bind to the consecutive repeat 16mers), the repeat sequences are being randomized separately.

17. For making tails with 3Q-dots, start with 3×16mer Tails 4Q-dots UWR.xls” document and randomize both unique and repeat tails separately. Then, place into master document and concatenate Q-dot Zip-complements. Paste back next to tail designation. 4Q Unique=1-4. 4Q Repeat=5-16.

18. Suitable complementary Q-tails according to the present invention include, without limitation, those shown in FIG. 97. FIG. 97 shows a list of 16 complementary Q-tails (SEQ ID NO:33 through SEQ ID NO:48). The tails are each 48mer and consist of three concatenated 16mer Q-Zips. The tails were constructed from the four complementary Q-Zips (SEQ ID NO:17 through SEQ ID NO:20) labeled QZ1-QZ4 in FIG. 96. Use of this format of encryption is illustrated in FIGS. 1-3. Take 3×16merTails 4Q-dot FIN.xls document, and concatenate Q-dot zip. Paste back next to tail designation.

19. Suitable Q-tails according to the present invention include, without limitation, those shown in FIG. 98. FIG. 98 shows a list of 16 Q-tails (SEQ ID NO:49 through SEQ ID NO:64). The tails are each 48mer and consist of three concatenated 16mer Q-Zips. The tails were constructed from the four Q-Zips (SEQ ID NO:1 through SEQ ID NO:4) labeled QZ1-QZ4 in FIG. 96.

20. Use of this format of encryption is illustrated in FIGS. 4-6.

21. Continue with “3×16mer Tails 8Q-dots UWR.xls” document, bring 4Q sequences to top and remove, and randomize both unique and repeat tails separately. Then place into master document and concatenate Q-dot zip-complements. Paste back next to tail designation. 8Q Unique=17-68. 8Q Repeat=69-112.

22. Suitable complementary Q-Zip tails according to the present invention include, without limitation, those shown in FIG. 99. FIG. 99 shows a list of 112 complementary Q-Zip tails (SEQ ID NO:33 through SEQ ID NO:144). The tails are each 48mer and consist of three concatenated 16mer Q-Zips. The tails were constructed from the eight complementary Q-Zips (SEQ ID NO:17 through SEQ ID NO:24) labeled QZ1-QZ8 in FIG. 96. Use of this format of encryption is illustrated in FIGS. 1-3. Take 3×16merTails 8Q-dot FIN.xls document, and concatenate Q-dot zip. Paste back next to tail designation.

23. Suitable Q-Zip tails according to the present invention include, without limitation, those shown in FIG. 100. FIG. 100 shows a list of 112 Q-Zip tails (SEQ ID NO:833 through SEQ ID NO:944). The tails are each 48mer and consist of three concatenated 16mer Q-Zips. The tails were constructed from the eight Q-Zips (SEQ ID NO:1 through SEQ ID NO:8) labeled QZ1-QZ8 in FIG. 96. Use of this format of encryption is illustrated in FIGS. 4-6.

24. Continue with “3×16mer Tails 12Q-dots UWR.xls” document, bring 8Q sequences to top and remove, and randomize both unique and repeat tails separately, (3×16mer Tails 12Q-8Q-dRand.xls). Then place into master document and concatenate Q-dot zip-complements. Paste back next to tail designation. 12Q Unique=113-276. 12Q Repeat=277-352.

25. Suitable complementary Q-Zip tails according to the present invention include, without limitation, those shown in FIG. 101. FIG. 101 shows a list of 352 complementary Q-Zip tails (SEQ ID NO:33 through SEQ ID NO:384). The tails are each 48mer and consist of three concatenated 16mer Q-Zips. The tails were constructed from the twelve complementary Q-Zips (SEQ ID NO:17 through SEQ ID NO:28) labeled QZ1-QZ12 in FIG. 96. Use of this format of encryption is illustrated in FIGS. 1-3. Take 3×16merTails 8Q-dot FIN.xls document, and concatenate Q-dot zip. Paste back next to tail designation.

26. Suitable Q-Zip tails according to the present invention include, without limitation, those shown in FIG. 102. FIG. 102 shows a list of 352 Q-Zip tails (SEQ ID NO:833 through SEQ ID NO:1184). The tails are each 48mer and consist of three concatenated 16mer Q-zips. The tails were constructed from the twelve Q-zips (SEQ ID NO:1 through SEQ ID NO:12) labeled QZ1-QZ12 in FIG. 96. Use of this format of encryption is illustrated in FIGS. 4-6.

27. Continue with “3×16mer Tails 16Q-dots UWR.xls” document, bring 12Q sequences to top and remove, and randomize both unique and repeat tails separately. Then place into master document and concatenate Q-dot Zip-complements. Paste back next to tail designation. 16Q Unique=353-692. 16Q Repeat=693-800. Suitable complementary Q-Zip tails according to the present invention include, without limitation, those shown in FIG. 103. FIG. 103 shows a list of 800 complementary Q-Zip tails (SEQ ID NO:33 through SEQ ID NO:832). The tails are each 48mer and consist of three concatenated 16mer Q-Zips. The tails were constructed from the sixteen complementary Q-Zips (SEQ ID NO:17 through SEQ ID NO:32) labeled QZ1-QZ16 in FIG. 96. Use of this format of encryption is illustrated in FIGS. 1-3. Take 3×16merTails 8Q-dot FIN.xls document, and concatenate Q-dot zip. Paste back next to tail designation.

28. Suitable Q-Zip tails according to the present invention include, without limitation, those shown in FIG. 104. FIG. 104 shows a list of 800 Q-Zip tails (SEQ ID NO:833 through SEQ ID NO:1632). The tails are each 48mer and consist of three concatenated 16mer Q-Zips. The tails were constructed from the sixteen Q-Zips (SEQ ID NO:1 through SEQ ID NO:16) labeled QZ1-QZ12 in FIG. 96. Use of this format of encryption is illustrated in FIGS. 4-6.

29. For making tails with 4Q-dots, start with “4×16mer Tails 8Q-dots UWR.xls” document and randomize both unique and repeat tails separately. Then place into master document and concatenate Q-dot Zip-complements. Paste back next to tail designation. 8Q Unique=1-70. 8Q Repeat=71-238.

30. Suitable complementary Q-Zip tails according to the present invention include, without limitation, those shown in FIG. 105. FIG. 105 shows a list of 238 complementary Q-Zip tails (SEQ ID NO:1633 through SEQ ID NO:1870). The tails are each 64mer and consist of four concatenated 16mer Q-Zips. The tails were constructed from the eight complementary Q-Zips (SEQ ID NO:17 through SEQ ID NO:24) labeled QZ1-QZ8 in FIG. 96.

31. Continue with “4×16mer Tails 12Q-dots UWR.xls” document and randomize both unique and repeat tails separately. Then place into master document and concatenate Q-dot zip-complements. Paste back next to tail designation. 12Q Unique=239-663. 12Q Repeat=664-1,155.

32. Suitable complementary Q-Zip tails according to the present invention include, without limitation, those shown in FIG. 106. FIG. 106 shows a list of 1155 complementary Q-Zip tails (SEQ ID NO:1633 through SEQ ID NO:2787). The tails are each 64mer and consist of four concatenated 16mer Q-Zips. The tails were constructed from the twelve complementary Q-Zips (SEQ ID NO:17 through SEQ ID NO:28) labeled QZ1-QZ12 in FIG. 96.

33. Continue with “4×16mer Tails 16Q-dots UWR.xls” document and randomize both unique and repeat tails separately. Then place into master document and concatenate Q-dot Zip-complements. Paste back next to tail designation. 16Q Unique=1,156-2,481. 16Q Repeat=2,482-3,500.

34. Suitable complementary Q-Zip tails according to the present invention include, without limitation, those shown in FIG. 107. FIG. 107 shows a list of 3500 complementary Q-Zip tails (SEQ ID NO:1633 through SEQ ID NO:5132). The tails are each 64mer and consist of four concatenated 16mer Q-Zips. The tails were constructed from the sixteen complementary Q-Zips (SEQ ID NO:17 through SEQ ID NO:32) labeled QZ1-QZ16 in FIG. 96.

### Example 4

### Construction of Q-Zip Tails with 2 Q-Dots Together on Each LDR Primer to Score LDR Product

One concept is to capture genomic DNA onto a support (e.g. magnetic beads), render it single stranded before or after capture, and then perform a single LDR directly on the captured single-stranded DNA target, effectively, capturing the LDR product onto the support. “Mini-cycling” during the LDR reaction (i.e. cycling between 65° C. and 75° C.) may be used to drive the LDR reaction toward completion, so that the LDR product accurately reflects the initial abundance of starting template. Excess LDR probes may be washed away. Each LDR probe would contain a 2×16mer tail, so that the LDR product is suitable for detection of a total of 4 Q-dots. These four Q-dots may be captured as hybridization reactions, chemical ligations, or biological ligation reactions.

1. Start with list of Q-dot zip and complements in Table 2 below. Sequences were generated as described in Example 1.

2. For making 2 tails with 2Q-dots each, start with “4×16mer Tails 8Q-dRand.xls” and reverse the randomization. The tails need to be re-randomized, but in sets of two to reflect the need to have two different discriminating primers ligating to the same common probe. For each tail, determine if numbers in first two columns (equivalent to common probe) are identical in pairs, label first “1” and second “2”. For odd tails, label “3”. Then sort by this designation. Tag “1” with random number (from 1-10,000), and use same set of random numbers to Tag “2”. Tag “3” with random number from 10,000 to 20,000. Sort by random number. Then place into master document and concatenate 2 tails of Q-dot zip-complements. List discriminating tails first (Columns 3 and 4), then complementary tails (Columns 1 and 2). By flipping the order of the Unique Singlets, they can be made into pairs. Paste back next to tail designation. 8Q Unique Pairs=1-60. 8Q Unique Singlets flipped into pairs=61-70 8Q Repeat Pairs=71-230. 8Q Unique Repeat for controls=231-238.

3. Suitable pairs of complementary Q-Zip tails according to the present invention include, without limitation, those shown in FIG. 108. FIG. 108 shows a list of 238 pairs of complementary Q-Zip tails. Each tail (SEQ ID NO:7593 through SEQ ID NO:7830; SEQ ID NO:11093 through SEQ ID NO:11330) is 32mer and consists of two concatenated Q-Zips. The tails were constructed from the eight complementary Q-Zips (SEQ ID NO:17 through SEQ ID NO:24) labeled QZ1-QZ8 in FIG. 96. Use of this format of encryption is illustrated in FIGS. 7-10, and 17-36.

4. Continue with “4×16mer Tails 12Q-8Q-dRan.xls” and reverse the randomization. The tails need to be re-randomized, but in sets of 2 to reflect the need to have two different discriminating primers ligating to the same common primer. For each tail, determine if numbers in first two columns (equivalent to common primer) are identical in pairs, label first “1” and second “2”. For odd tails, label “3”. Then sort by this designation. Tag “1” with random number (from 1-10,000), and use same set of random numbers to Tag “2”. Tag “3” with random number from 10,000 to 20,000. Sort by random number. Then place into master document and concatenate 2 tails of Q-dot zip-complements. List discriminating tails first (Columns 3 and 4), then complementary tails (Columns 1 and 2). Paste back next to tail designation. 12Q Unique Pairs=239-646. 12Q Unique Singlets flipped into pairs=647-662. 12Q Unique Singlet=663. 12Q Repeat Pairs=664-1155. 12Q Unique Repeat for controls=none.

5. Suitable pairs of complementary Q-Zip tails according to the present invention include, without limitation, those shown in FIG. 109. FIG. 109 shows a list of 1155 pairs of complementary Q-Zip tails. Each tail (SEQ ID NO:7593 through SEQ ID NO:8747; SEQ ID NO:11093 through SEQ ID NO:12247) is 32mer and consists of two concatenated Q-Zips. The tails were constructed from the twelve complementary Q-Zips (SEQ ID NO:17 through SEQ ID NO:28) labeled QZ1-QZ12 in FIG. 96. Use of this format of encryption is illustrated in FIGS. 7-10, and 17-36.

6. Continue with “4×16mer Tails 16Q-12Q-dRan.xls” and reverse the randomization. The tails need to be re-randomized, but in sets of 2 to reflect the need to have two different discriminating probes ligating to the same common probe. For each tail, determine if numbers in first two columns (equivalent to common probe) are identical in pairs, label first “1” and second “2”. For odd tails, label “3”. Then sort by this designation. Tag “1” with random number (from 1-10,000), and use same set of random numbers to Tag “2”. Tag “3” with random number from 10,000 to 20,000. Sort by random number. Then place into master document and concatenate 2 tails of Q-dot zip-complements. List discriminating tails first (Columns 3 and 4), then complementary tails (Columns 1 and 2). Paste back next to tail designation. 16Q Unique Pairs=1146-2457. 16Q Unique Singlets flipped into pairs=2458-2479. 16Q Unique singlet=2480 16Q Repeat Pairs=2481-3496.

16Q Unique Repeat for controls=3497-3500.

7. Suitable pairs of complementary Q-Zip tails according to the present invention include, without limitation, those shown in FIG. 110. FIG. 110 shows a list of 3500 pairs of complementary Q-Zip tails. Each tail (SEQ ID NO:7593 through SEQ ID NO:11092; SEQ ID NO:11093 through SEQ ID NO:14592) is 32mer and consists of two concatenated Q-Zips. The tails were constructed from the sixteen complementary Q-Zips (SEQ ID NO:17 through SEQ ID NO:32) labeled QZ1-QZ16 in FIG. 96.

### Example 5

### Construction of Q-Zip Tails With 2 Q-dots Ligated Together On Each LDR Probe To Score LDR Product

Q-Zip tails with 2 Q-dots ligated together on each LDR probe are constructed as follows:

1. Start with list of Q-dot Zips and complements in Table 3 below. Sequences were generated as described in Example 2.

2. One possibility is to ligate the Q-dot Zips together onto the tails. There are a number of different ways this may be accomplished, but the simplest format would have the Q-dots attached to the non-ligating ends of each 16mer sequence. As such, there may be a need to either make two Q-dots versions for each 16mer; one with the attachment on the 3′ end, the other with the attachment on the 5′ end. Alternatively, by reducing the list of tails, so that two will be “even” and two will be “odd” the Q-dot will always hybridize in the correct orientation. In order to make this list, start with “4×16mer Tails 8Q-dRand.xls” and reverse the randomization. Calculate the sum of the four columns and determine if the sum is even (=True) or Odd (=False). Sort, and remove all odd sums. Thus, a given row will have two odd and two even numbers (what we are looking for), or all even, or all odd. Test for all odd numbers in the first column, all even numbers in the second column. Move to bottom, and remove from list. For each tail, determine if numbers in first two columns (equivalent to common probe) are identical in pairs (one odd, the other even), label first “1” and second “2”. For odd tails, label “3”. Then sort by this designation. Tag “1” with random number (from 1-10,000), and use same set of random numbers to Tag “2”. Look at Tag “3”. If it has two even or two odd numbers in the first two columns, mix columns until one even and one odd in first two columns. Then proceed with labeling these “1” and “2”. Leftovers get 3 designation, and try again, (label 11 and 12 until only left with 13's, tag with random number from 10,000 to 20,000. Sort by random number.

3. Now one needs to orient the pairs so that odd numbers go in the first row, even in the second, odd in the third, and even in the last row. Then place into master document and concatenate 2 tails of Q-dot zip-complements. List discriminating tails first (Columns 3 and 4), then complementary tails (Columns 1 and 2). Once sequences are concatenated, discriminating tails are listed under “First 2XQ-Zip Lig tail complement”, then common tails are listed under “Second 2XQ-Zip Lig tail complement”. 8Q Unique Pairs=1-34. 8Q Unique Singlets for controls=35-36. 8Q Repeat Pairs=37-84.

4. Suitable pairs of complementary Q-Zip tails according to the present invention include, without limitation, those shown in FIG. 111. FIG. 111 shows a list of 84 pairs of complementary Q-Zip tails. Each tail (SEQ ID NO:5133 through SEQ ID NO:5126; SEQ ID NO:6363 through SEQ ID NO:6446) is 32mer and consists of two concatenated Q-Zips. The tails were constructed from the eight complementary Q-Zips (SEQ ID NO:17 through SEQ ID NO:24) labeled QZ1-QZ8 in FIG. 96. Use of this format of encryption is illustrated in FIGS. 8, 10, 18, 20, 22, 24, 26, 28, 30, 32, 34, and 36.

5. Continue with the next set, orienting the pairs so that odd numbers go in the first row, even in the second, odd in the third, and even in the last row. Then place into master document and concatenate 2 tails of Q-dot zip-complements. List discriminating tails first (Columns 3 and 4), then complementary tails (Columns 1 and 2). Once sequences are concatenated, discriminating tails are listed under “First 2XQ-Zip Lig tail complement”, then common tails are listed under “Second 2XQ-Zip Lig tail complement”. 12Q Unique Pairs=85-268. 12Q Unique Singlets for controls=268-273. 12Q Repeat Pairs=274-393. 12Q Repeat Singlets for controls=394-403.

6. Suitable pairs of complementary Q-Zip tails according to the present invention include, without limitation, those shown in FIG. 112. FIG. 112 shows a list of 403 pairs of complementary Q-Zip tails. Each tail (SEQ ID NO:5133 through SEQ ID NO:5535; SEQ ID NO:6363 through SEQ ID NO:6765) is 32mer and consists of two concatenated Q-Zips. The tails were constructed from the twelve complementary Q-Zips (SEQ ID NO:17 through SEQ ID NO:28) labeled QZ1-QZ12 in FIG. 96. Use of this format of encryption is illustrated in FIGS. 8, 10, 18, 20, 22, 24, 26, 28, 30, 32, 34, and 36.

7. Continue with the next set, orienting the pairs so that odd numbers go in the first row, even in the second, odd in the third, and even in the last row. Then place into master document and concatenate 2 tails of Q-dot zip-complements. List discriminating tails first (Columns 3 and 4), then complementary tails (Columns 1 and 2). Once sequences are concatenated, discriminating tails are listed under “First 2XQ-Zip Lig tail complement”, then common tails are listed under “Second 2XQ-Zip Lig tail complement”. 16Q Unique Pairs=404-955. 16Q Unique Singlets for controls=956-962. 16Q Repeat Pairs=963-1218. 16Q Repeat Singlets for controls=1219-1230.

8. Suitable pairs of complementary Q-Zip tails according to the present invention include, without limitation, those shown in FIG. 113. FIG. 113 shows a list of 1230 pairs of complementary Q-Zip tails. Each tail (SEQ ID NO:5133 through SEQ ID NO:6362; SEQ ID NO:6363 through SEQ ID NO:7592) is 32mer and consists of two concatenated Q-Zips. The tails were constructed from the sixteen complementary Q-Zips (SEQ ID NO:17 through SEQ ID NO:32) labeled QZ1-QZ16 in FIG. 96. Use of this format of encryption is illustrated in FIGS. 8, 10, 18, 20, 22, 24, 26, 28, 30, 32, 34, and 36.

### Example 6

### Construction of Q-Zip Tails with 2 Q-dots Together on One LDR Probe (and Universal Array Zipcode on Other LDR Probe) to Score LDR Product

Another concept is to retain the original universal array architecture, but take advantage of the multiple colors made available by using a combination of Q-dots. Here the LDR products will have a traditional 24mer zipcode complementary sequence on one side, for capture on the universal array, and a 32mer Q-dot zip complementary sequence on the other side to allow for capture of two Q-dots. By judicious choice of Q-dots, sequences may be chosen that allow one to distinguish true signal from noise at a given address.

1. Start with list of Q-dot zip and complements in Table 2 below. Sequences were generated as described in Example 1.

2. For making single tails with 2Q-dots each, start with “3×16mer Tails 16Q-dotsUWR.xls” and save the listing of all 120 pairs of 16 items, each used only once (first column has lower number). Then make subcategories of all pairs with numbers <5 (=zips 1-6), <9 (=zips 7-28), and <13 (=zips 29-66), and remaining=67-120.

3. Resave as “16mer SingleTail16Q-dotsRAN.xls” for randomization. For zips 1-6, tag with random numbers 1-10,000. For zips 7-28, tag with random numbers 10,000-20,000. For zips 29-66, tag with random numbers 30,000-40,000. For zips 67-120, tag with random numbers 50,000-60,000. Sort by random number. Then place into master document and concatenate single tail of Q-dot zip-complements.

4. Suitable single complementary Q-Zip tails according to the present invention include, without limitation, those shown in FIG. 114. FIG. 114 shows a list of 120 single complementary Q-Zip tails. Each tail (SEQ ID NO:14593 through SEQ ID NO:14712) is 32mer and consists of two concatenated Q-Zips. The tails were constructed from the sixteen complementary Q-Zips (SEQ ID NO:17 through SEQ ID NO:32) labeled QZ1-QZ16 in FIG. 96. Two Q-dots will hybridize with each tail, allowing detection of two Q-dots per tail. Use of this format of encryption is illustrated in FIGS. 37-40, 45-53. Take 16mer SingleTail16Q-dotsFIN.xls document, and concatenate Q-dot zip. Paste back next to tail designation.

5. Suitable single Q-Zip tails according to the present invention include, without limitation, those shown in FIG. 115. FIG. 115 shows a list of 120 single Q-Zip tails. Each tail (SEQ ID NO:14713 through SEQ ID NO:14832) is 32mer and consists of two concatenated Q-Zips. The tails were constructed from the sixteen Q-Zips (SEQ ID NO:1 through SEQ ID NO:16) labeled QZ1-QZ16 in FIG. 96. Two Q-dots will hybridize with each tail, allowing detection of two Q-dots per tail. Use of this format of encryption is illustrated in FIGS. 11-16, 41-44, and 54-62.

### Example 7

### Construction of Q-Zip Tails with 2 Q-Dots Ligated Together on One LDR Probe (and Universal Array Zipcode on Other LDR Probe) to Score LDR Product

Q-Zip tails with Q-dots ligated together on one LDR probe:

1. Start with list of Q-dot Zip and complements in Table 2 below. (Q-dot zip+Complement Tm65.5-67.4). Sequences were generated as described in Example 2.

2. One possibility is to ligate the Q-dot Zips together onto the tails. There are a number of different ways this may be accomplished, but the simplest format would have the Q-dots attached to the non-ligating ends of each 16mer sequence. As such, there may be a need to either make two Q-dots versions for each 16mer; one with the attachment on the 3′ end, the other with the attachment on the 5′ end. Alternatively, by reducing the list of tails, so that the first position will be “odd” and the second “even” the Q-dot will always hybridize in the correct orientation. Manually make such a list from 1-16, with odd numbers in first row, even numbers in second row. Since the rows are segregated into odd and even, there is no risk of the same two numbers being used in opposite orientation (i.e. 1-8 vs. 8-1). Then make subcategories of all pairs with numbers <5 (=zips 1-4), <9 (=zips 5-16), and <13 (=zips 17-36), and remaining=37-64.

3. Resave as “16mer LigSingleTail 16Q-Ran.xls” for randomization. For zips 1-4, tag with random numbers 1-10,000. For zips 5-16, tag with random numbers 10,000-20,000. For zips 17-36, tag with random numbers 30,000-40,000. For zips 37-64, tag with random numbers 50,000-60,000. Sort by random number. Then place into master document and concatenate single tail of Q-dot zip-complements.

4. Suitable single complementary Q-Zip tails according to the present invention include, without limitation, those shown in FIG. 116. FIG. 116 shows a list of 64 single complementary Q-Zip tails. Each tail (SEQ ID NO:14833 through SEQ ID NO:14896) is 32mer and consists of two concatenated Q-Zips. The tails were constructed from the sixteen complementary Q-Zips (SEQ ID NO:17 through SEQ ID NO:32) labeled QZ1-QZ16 in FIG. 96. Two Q-dots will hybridize with each tail, allowing detection of two Q-dots per tail. Upon hybridization, the Q-dots will ligate to each other in the presence of a thermostable ligase. Use of this format of encryption is illustrated in FIGS. 38, 40, 46, 47, 49, 50, 52, and 53. Take 16mer SingleTail16Q-dotsFIN.xls document, and concatenate Q-dot zip. Paste back next to tail designation.

5. Suitable single Q-Zip tails according to the present invention include, without limitation, those shown in FIG. 117. FIG. 117 shows a list of 64 single Q-Zip tails. Each tail (SEQ ID NO:14897 through SEQ ID NO:14960) is 32mer and consists of two concatenated Q-Zips. The tails were constructed from the sixteen Q-Zips (SEQ ID NO:1 through SEQ ID NO:16) labeled QZ1-QZ16 in FIG. 96. Two Q-dots will hybridize with each tail, allowing detection of two Q-dots per tail. Upon hybridization, the Q-dots will ligate to each other in the presence of a thermostable ligase. Use of this format of encryption is illustrated in FIGS. 12, 14 and 16, 42, 44, 55, 56, 58, 59, 61, and 62.

### Example 8

### T-Zip Tails Construction

Each T-zip-code sequence is composed of four tetramers (designed as described in the following) so that the full-length 16-mers have similar Tm values. The 256 (44) possible combinations in which four bases can be arranged as tetramers were reduced to a set of 36 for use in standard universal array zip codes and Q-Zips; these were chosen so that each tetramer differs from all others by at least two bases (See Table 2). For construction of T-zip sequences, a different set of tetramers were chosen, with the aim of selecting tetramers with 2 or 3 G+C bases, that are substantially different than the set of tetramers used to construct Q-Zips and universal zip codes (see Table 5, below). Tetramer complements, as well as tetramers that would result in self-pairing or hairpin formation of the zip-codes, were eliminated. Furthermore, tetramers that were palindromic, e.g., TCGA, or repetitive, e.g., CACA, were excluded. The indicated set of twenty tetramers represents just one of the possible sets that can be created. In the following description, a tetramer is referred to by its designation in the first column of Table 5. For example, #5=GGAA. In the set of T-tetramers chosen, only two are related to those in Table 1, T-tetramer #3 AGAC is the complement of #19, and T-tetramer #21 GGTC is the complement of #32.

TABLE 5

Tetramer sequences and complements
used to construct T-zip sequences

| | Tetramer | Tetramer | |
|---|---|---|---|
| T-Tetramer | sequence | complement | |
| designation | 5′-3′ | 5′-3′ | G + C bases |
| 1 | ACCA | TGGT | 2 |
| 2 | ACTG | CAGT | 2 |
| 3 | AGAC | GTCT | 2 |
| 4 | CCAA | TTGG | 2 |
| 5 | GGAA | TTCC | 2 |
| 6 | TCAC | GTGA | 2 |
| 7 | TGCT | AGCA | 2 |
| 8 | ACGC | GCGT | 3 |
| 9 | AGCG | CGCT | 3 |
| 10 | CACC | GGTG | 3 |
| 11 | CAGG | CCTG | 3 |
| 12 | CCTC | GAGG | 3 |
| 13 | CGAG | CTCG | 3 |
| 14 | CGGA | TCCG | 3 |
| 15 | GACG | CGTC | 3 |
| 16 | GAGC | GCTC | 3 |
| 17 | GCAG | CTGC | 3 |
| 18 | GCCT | AGGC | 3 |
| 19 | GCGA | TCGC | 3 |
| 20 | GGGT | ACCC | 3 |
| 21 | GGTC | GACC | 3 |
| 22 | GTCC | GGAC | 3 |
| 23 | GTGG | CCAC | 3 |
| 24 | TGGC | GCCA | 3 |

1. Create list 1-576 of all variations of 24×24 tetramers.

2. Compute Tm values of 576 8-mers in Oligo 6.4 and sort according to Tm.

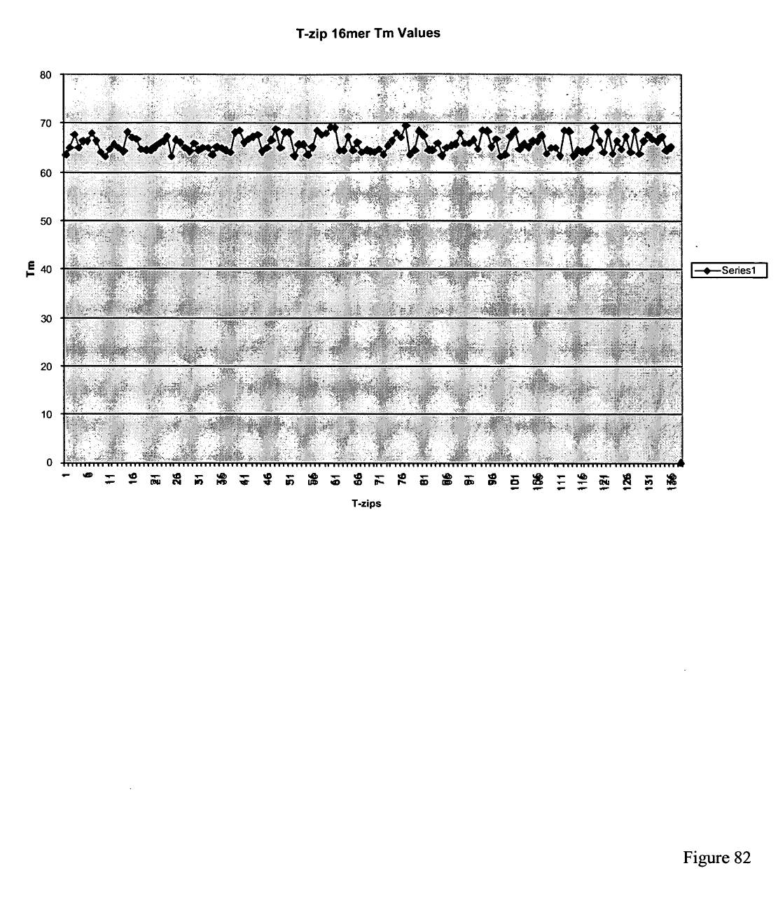

3. Take the 300 8mers with the highest Tm Values (between 6.6 and 25) randomize within Tm value, split in half, reverse one half, and recombine to form 150 16 mers. Calculate Tm values of these=61° C. to 69.5° C. Remove sequences that use a tetramer 3 times. Remove lower Tm oligos so list has 136 members with Tm values from 63° C. to 69.5° C. Attach random number generator and randomize these 16 mer T-Zip sequences. (T-zip Tetramer Tm sortRan2.xls). Plot Tm values (FIG. 82). Also determine complement sequence.

4. Suitable T-Zip codes according to the present invention include, without limitation, those shown in FIG. 118. FIG. 118 shows a list of 136 T-Zip code sequences (SEQ ID NO:14961 through SEQ ID NO:15096), and their 136 complements (SEQ ID NO:15097 through SEQ ID NO:15232). The T-Zips are each 16mer and were constructed.

### Example 9

### Adding Q-Zips to T-Zip Tails for Universal Detection

A set of 136 T-Zip sequences of 16 bases each, compatible for use with either Q-Zips, universal Zip codes, or both, with high Tm values have been designed in Example 8. Each T-Zip is equivalent to two Q-Zips, where each Q-Zip can be from 1 to 16, and the order does not matter ((16×15)/2+16=136). These 136 T-Zips may be combined to form a 32mer sequence (9180 combinations) that may be attached to an oligonucleotide, bead, or array. The presence of this 32mer sequence allows deconvoluting of the original signal by ligating on probes that contain the T-Zip sequences at the ligation junction, and two Q-Zip complements at the non-ligating end of each T-Zip.

1. Starting with the set of 136 different ways to have 2 Q-Zips (from 1-16) combined, sort by combinations less than 5, 9, and 13. Create second list of 136 where order of Q-Zips is inverted. These lists are combined with the unique 136 different T-Zip sequences.

2. Suitable T-Zip codes according to the present invention include, without limitation, those shown in FIG. 119. FIG. 119 shows the list of 136 T-Zip code sequences (SEQ ID NO:14961 through SEQ ID NO:15096) and their 136 complements (SEQ ID NO:15097 through SEQ ID NO:15232) shown in FIG. 118. Also shown are the Q-Zip and reverse Q-Zip numbers. Provide each pair with a unique identifier=(Position 1+Position 2)*40+(Position 2-Position 1). Combine columns and sort by unique identifier, from 80 to 1280. Those examples where a given Q-Zip was used twice will be repeated.

3. Create a new document that contains the 136 different T-Zip sequences. Combine the numbers with the unique identifiers, such that each T-Zip number and corresponding T-Zip sequence is listed at the position corresponding to the unique identifiers.

4. The expanded list serves as a template to interconvert pre-existing Q-Zip lists into Q-Zip/T-Zip lists.

5. Start with the 2×16mer 2Tails 16Q-dotFIN.xls document. Insert 136 Expand list and concatenate Q-Zip complement with T-Zip sequence, with a spacer in between. For the initial LDR probe, the 2 T-Zip portions are fused together. For the upstream probe, the sequence is 2×Q-Zip complement-spacer-T-Zip. For the downstream (phosphorylated) probe, the sequence is T-Zip-spacer-2×Q-Zip-complement.

6. Suitable T-Zip tails according to the present invention include, without limitation, those shown in FIG. 120. FIG. 120 shows a list of 3500 T-Zip tails (SEQ ID NO:15233 through SEQ ID NO:18732) (initial LDR probes). Each tail is 32mer and consists of two concatenated T-Zips. The tails were constructed from the 136 T-Zips (SEQ ID NO:14961 through SEQ ID NO:15096) shown in FIG. 119.

7. Suitable upstream and downstream probes according to the present invention include, without limitation, those shown in FIG. 121. FIG. 121 shows a list of 3500 upstream and 3500 downstream probes. Each upstream probe consists of two concatenated Q-Zips (SEQ ID NO:19963 through SEQ ID NO:23462) followed by a spacer and a T-Zip (SEQ ID NO:23463 through SEQ ID NO:26962). Each downstream probe consists of one T-Zip (SEQ ID NO:26963 through SEQ ID NO:30462) followed by a spacer and two concatenated complementary Q-Zips (SEQ ID NO:30463 through SEQ ID NO:33962). The probes were constructed from the 136 T-Zips (SEQ ID NO:14961 through SEQ ID NO:15096) shown in FIG. 119 and the 16 complementary Q-Zips (SEQ ID NO:17 through SEQ ID NO:32) labeled QZ1-QZ16 in FIG. 96. Sequence identification numbers refer to the nucleotide sequence immediately following or immediately preceding the sequence identification number, as applicable. Use of this format of encryption is illustrated in FIGS. 63-71.

8. Repeat the above procedure with document 2×16mer 2LigTails16Q-FIN.xls to yield appropriate Q-Zip/T-Zip oligonucleotide sequences.

9. Suitable T-Zip tails according to the present invention include, without limitation, those shown in FIG. 122. FIG. 122 shows a list of 1230 T-Zip tails (SEQ ID NO:18733 through SEQ ID NO:19962) (initial LDR primers). Each tail is 32mer and consists of two concatenated T-Zips. The tails were constructed from the 136 T-Zips (SEQ ID NO:14961 through SEQ ID NO:15096) shown in FIG. 119.

10. Suitable upstream and downstream probes according to the present invention include, without limitation, those shown in FIG. 123. FIG. 123 shows a list of 1230 upstream and 1230 downstream probes. Each upstream probe consists of one ligated Q-Zip tail (SEQ ID NO:33963 through SEQ ID NO:35192) followed by a spacer and a T-Zip (SEQ ID NO:35193 through SEQ ID NO:36422). Each downstream probe consists of one T-Zip (SEQ ID NO:36423 through SEQ ID NO:37652) followed by a spacer and one complementary ligated Q-Zip tail (SEQ ID NO:37653 through SEQ ID NO:38882). The probes were constructed from the 1230 T-Zips tails (SEQ ID NO:18733 through SEQ ID NO:19962) shown in FIG. 122, and the 1230 Q-Zip tails (SEQ ID NO:5133 through SEQ ID NO:6362) and 1230 complementary Q-Zip tails (SEQ ID NO:6363 through SEQ ID NO:7592) shown in FIG. 113. Sequence identification numbers refer to the nucleotide sequence immediately following or immediately preceding the sequence identification number, as applicable. Use of this format of encryption is illustrated in FIGS. 64, 65, 67, 68, 70, and 71.

### Example 10

### Whole Genome Amplification

Whole genome amplification is a method of isothermal DNA amplification that is capable of producing in the order of 50 μg of DNA from 100 ng starting material in a 50 μl reaction (Lage et al., “Whole Genome Analysis of Genetic Alterations in Small DNA Samples Using Hyperbranched Strand Displacement Amplification and Array-CGH,” Genome Res. 13:294-307 (2003), which is hereby incorporated by reference in its entirety). The technique has several advantages over the polymerase chain reaction. For example, amplification bias in the product is low (reported to be 3 fold as opposed to 4 to 6 orders of magnitude for PCR) so representation across the genome is highly uniform; amplification products have a high average size; and primer interference and dimerization, which can be particularly problematic when PCR is performed on small amounts of product, are avoided. The technique can be used, for example, when limited quantities of tumor samples are available from a needle biopsy and the amount of DNA that is present is insufficient for detailed analysis.

Semi-random octamer oligonucleotide primers were designed so that the two nucleotides at the 3′ end were known. Two sets of primers were synthesized (Integrated DNA Technologies), one with 3′ AA and another with 3′ CC, with a phosphorothioate linkage between these nucleotides (designated as “s”), to confer resistance to enzymatic digestion. Two nitroindole bases (designated as I(NO₂)) were included at the 5′ end of the primers (Lage et al., “Whole Genome Analysis of Genetic Alterations in Small DNA Samples Using Hyperbranched Strand Displacement Amplification and Array-CGH,” Genome Res. 13:294-307 (2003), which is hereby incorporated by reference in its entirety):

5′-I(NO₂) - I(NO₂) NNN NNN AsA -3′
and
5′-I(NO₂) - I(NO₂) NNN NNN CsC -3′

1.2 μM of one semi-random octamer set was added to 50-100 ng human genomic DNA (Roche) in a total volume of 7.5 μl. These DNAs were heated for 10 minutes at 960 C and were cooled on ice.

A master mix was prepared, containing 5 μl of 2.5 mMdNTPs (Roche), 2 μl DMSO (Sigma), 16 units Bst polymerase (New England Biolabs), 5 μl 10× buffer (consisting of 100 mM tricine, 100 mM KCl, 50 mM (NH₄)₂SO₄, 52.5 mM MgCl₂, 1% Triton X-100, pH adjusted to 9.1-9.15 using NaOH; all reagents supplied by Sigma) and water to a total volume of 43.5 μl for each reaction. Following a brief centrifugation of the DNAs, 43.5 μl of master mix was added to each reaction, giving a total volume of 50 μl. Tubes were incubated at 50° C. for 4 hours, followed by 96° C. for 10 minutes.

During whole genome amplification, semi-random hexamer oligonucleotides anneal to the template DNA and are amplified by Bst polymerase. Bst polymerase is suited to this task, because it is highly processive and has strong displacement activity. Product DNA molecules are more readily dislodged from the template, allowing new primers to anneal. Although the optimal temperature of this enzyme is 65° C., yields of the amplified product were negligible at this temperature, presumably due to primer dissociation. Optimal compromise between enzyme activity and primer annealing was obtained at 500 C. Following incubation at 50° C. for 4 hours, a hyperbranched product is formed that represents a several hundred-fold amplification of the template DNA.

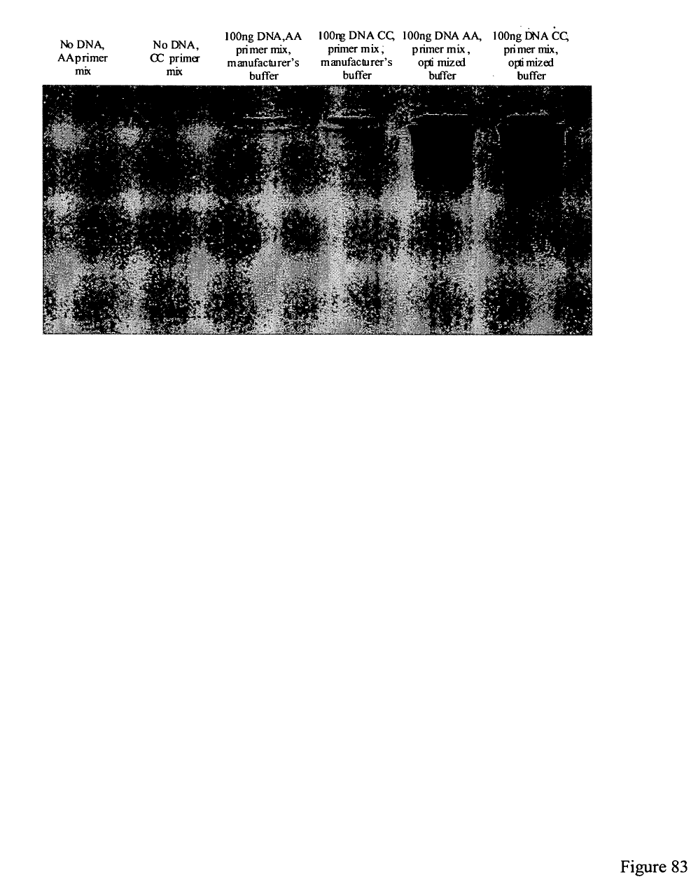

Product yields from amplification of 100 ng human genomic DNA are shown in FIG. 83. Amplification products using no DNA template were run in the left hand two lanes; these appear to be blank, showing that no detectable primer dimers are formed under the conditions used. The right hand lanes show amplification products using the optimized buffer described above; the yield obtained is considerable greater than with the manufacturer's buffer (center lanes). It can be seen that the yield obtained from the semi-random octamers ending in 3′ CC is higher than that obtained from the 3′ AA primers. This presumably reflects the higher average melting temperature of the 3′ CC primers.

### Example 11

### Testing of Synthetic Oligonucleotides for Resistance to Different Exonucleases

As shown in FIGS. 1-71 scoring of ligation products may be achieved by using Q-dots. It is important to minimize any non-specific background noise in the SNP detection that would arise from non-specific binding of unligated LDR oligonucleotide probes. These must be removed or destroyed by exonuclease digestion following the ligation step. For removal by exonucleases, both the allele-specific and common oligonucleotide probes should contain protecting groups on their 5′- and 3′-end, respectively, so that only ligation products are rendered resistant to exonuclease digestion, while unligated LDR oligonucleotide probes are targeted by 5′→3′ and 3′ 5′ exonucleases. With this aim, nine fluorescently labeled oligonucleotides were designed with various protecting groups, listed in Table 6, below, and group(s) resistant to exonuclease digestion were determined.

TABLE 6

List of the synthetic substrates (24-mers) used in the
Exonuclease Test, each carrying one or several protecting
group(s) (shown in bold) on their 5′ and/or 3′ end.

| | |
|---|---|
| Oligo- | |
| nucleotide name: | Oligonucleotide sequence |

| | | |
|---|---|---|
| XS 3′F1 | 5′ CGCAGATTTTGCGCTGGATTTCAA Fluorescein 3′ | (SEQ ID NO: 38887) |
| XS 5′F2 | 5′ Fluorescein -CGCAGATTTTGCGCTGGATTTCAA 3′ | (SEQ ID NO: 38887) |
| XRS 4 | 5′ Biotin TEG - Spacer 18- | (SEQ ID NO: 38887) |
| | CGCAGATTTTGCGCTGGATTTCAA Fluorescein- | |
| | Spacer 18- spacer C3 3′ | |
| XRS 5 | 5′ Amino-Modifier C3- | (SEQ ID NO: 38887) |
| | CGCAGATTTTGCGCTGGATTTCAA Fluorescein- Spacer | |
| | 18- Biotin TEG 3′ | |
| XRS 6 | 5′ Biotin TEG -UOMeCGCAGATTTTGCGCTGGATTTCAA | (SEQ ID NO: 38887) |
| | Fluorescein-UOMe-Biotin TEG 3′ | |
| XRS 7 | 5′ UOMe-Biotin TEG-CGCAGATTTTGCGCTGGATTTCAA | (SEQ ID NO: 38887) |
| | Fluorescein-Biotin TEG UOMe 3′ | |
| XRS 8 | 5′ Biotin TEG -(UOMe)4- | (SEQ ID NO: 38887) |
| | CGCAGATTTTGCGCTGGATTTCAA Fluorescein- | |
| | (UOMe)4-Biotin TEG 3′ | |
| XRS 9 | 5′ (UOMe)4-Biotin TEG- | (SEQ ID NO: 38887) |
| | CGCAGATTTTGCGCTGGATTTCAA Fluorescein- | |
| | (UOMe)4 3′ | |
| XRS 10 | 5′ (UOMe)4-Biotin TEG-CGCAGATTTTGC-Spacer | (SEQ ID NO: 38887) |
| | 18-GCTGGATTTCAA | |
| | Fluorescein Biotin TEG -(UOMe)4 3′ | |

Fluorescein = green fluorescent dye
Biotin TEG (5′ or branched) = 1-Dimethoxytrityloxy-3-O-(N-biotinyl-3-aminopropyl)-triethyleneglycolyl-glyceryl-2-O-(2-cyanoethyl)-(N,N-diisopropyl)-phosphoramidite
Biotin TEG (3′) = 1-Dimethoxytrityloxy-3-O-(N-biotinyl-3-aminopropyl)-triethyleneglycolyl-glyceryl-2-O-succinyl-long chain alkylamino-CPG
Spacer C3 or 18 = spacer phosphoramidites are used to insert a spacer arm in an oligonucleotide, and may also act as blockers of exonuclease activity
5′-Amino-Modifier C3 = designed to functionalize the 5′-terminus of the oligonucleotide with an amino-group attached to a triple carbon chain
UOMe = 2′-O-methyl-uridine riboside

The following commercially available exonucleases were tested:
5′→3′ Lambda exonuclease, whose preferred substrate is 5′-phosphorylated double-stranded DNA;
5′→3′ T7 exonuclease, whose preferred substrate is duplex DNA;
3′→5′ E. coli Exonuclease I, which catalyzes the removal of nucleotides from single-stranded DNA;
3′→5′ Exonuclease III, which catalyzes the removal of nucleotides from double-stranded DNA; and
5′→3′ and 3′→5′ E. coli Exonuclease VII, which is an exclusively single-strand directed enzyme.

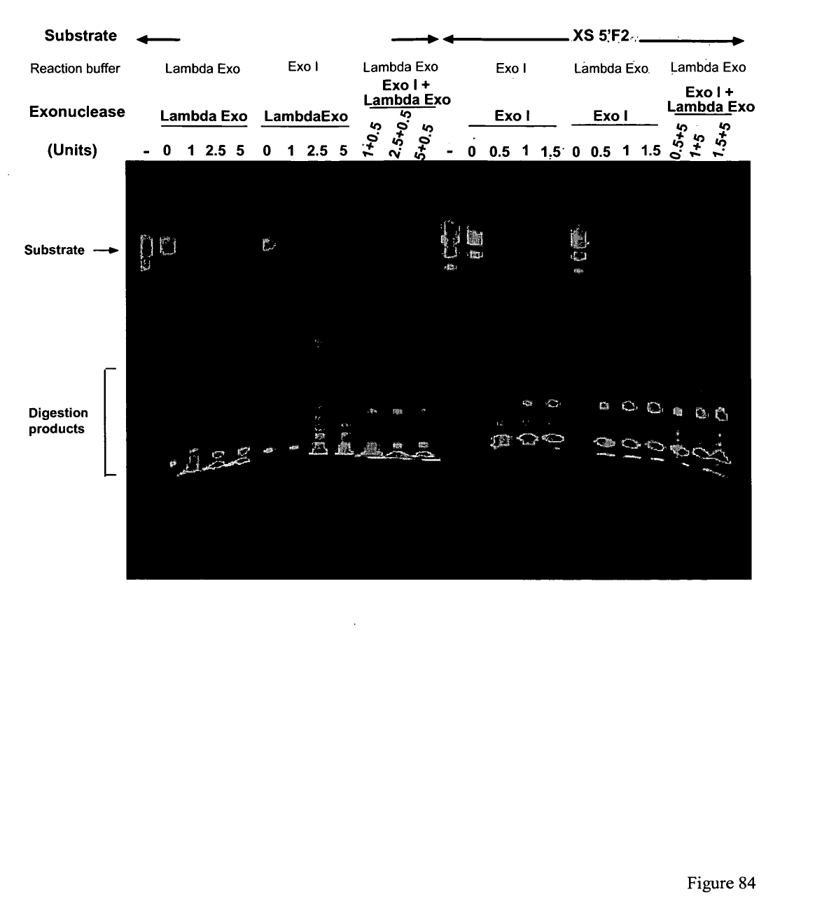

The results of these experiments are shown in FIGS. 84-88. A summary of these results is presented in Table 7, below.

TABLE 7
Test of synthetic oligonucleotides for resistance to different exonucleases.

| | | Lambda | | | | |
|---|---|---|---|---|---|---|
| | Oligo | Exo | T7 Exo | Exo I | Exo III | Exo VII |
| Synthetic oligonucleotides | name | 5'−>3′ | 5′−>3′ | 3′−>5′ | 3′−>5′ | 5′−>3′ & 3′−>5′ |
| 5′ --- F1 3′ | XS 3′F1 | no(*) | partial | untested | untested | no |
| 5′ F1---- 3′ | XS 5′F2 | untested | untested | no | no | no |
| 5′ B-S18-- -F1-S18-Sc3 3′ | XRS 4 | yes | yes | yes | no | no |
| 5′ NH2---F1-S18-B 3′ | XRS 5 | yes | untested | partial | no | no |
| 5′ B-UOMe----F1-UOMe-B 3′ | XRS 6 | partial | yes | yes | partial | no |
| 5′ UOMe-B----F1-B-UOMe 3′ | XRS 7 | partial | untested | yes | partial | no |
| 5′ B-(UOMe)4---F1-(UOMe)4-B 3′ | XRS 8 | yes | yes | yes | partial | no |
| 5′ (UOMe)4-B---F1-(UOMe)4 3′ | XRS 9 | partial | yes | yes | partial | no |
| 5′ (UOMe)4-B--S18-F1-B-(UOMe)4 3′ | XRS 10 | yes | partial | partial | no | no |

yes = Fully resistant to exonuclease digestion.
partial = Partially resistant to exonuclease digestion.
no = Sensitive to exonuclease digestion.

Among the nine synthetic substrates tested for exonuclease resistance, control oligos referred to as XS 3′F1 and XS 5′F2 were designed with a 5′- and a 3′-free terminus, respectively. Prior to exonuclease digestion, the 5′ end of XS 3′ F1 was phosphorylated by T4 polynucleotide kinase. As anticipated, 5′-phosphorylated XS 3′F1 is fully digested by 5′→3′ Lambda exonuclease in Lambda Exo digestion buffer. In the presence of the enzyme, the band corresponding to the substrate is no longer detected, yet digestion products appear at the lower part of the gel, as shown in FIG. 84. One unit of Lambda exonuclease is sufficient to digest the control oligo to completion, whether the reaction is performed in Lambda Exo buffer or in Exonuclease I buffer, both in the presence and absence of Exonuclease I.

Similarly, XS 5′F2 is digested by Exonuclease I in either Exonuclease I buffer or in Lambda Exo buffer. One unit results in complete digestion in either buffer, both in the presence and absence of Lambda exonucleases, as shown in FIG. 84.

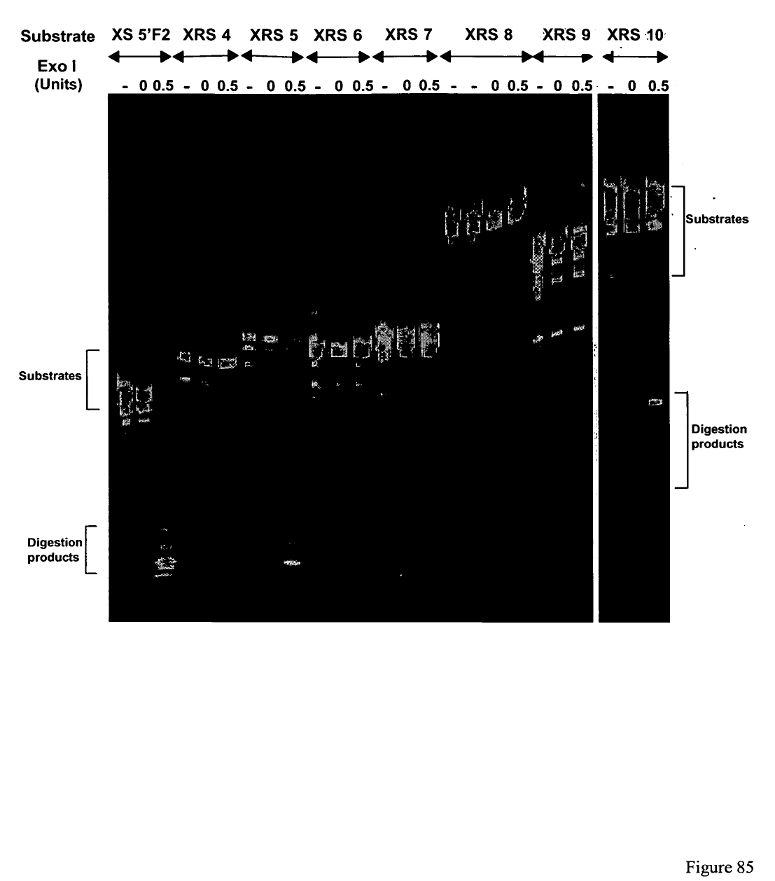

Exo I digestion tests were subsequently conducted on oligos XRS 4 through XRS 10, as well as on XS 5′F2 control oligo, as shown in FIG. 85. As previously observed, XS 5′F2 is digested by 0.5 unit Exo I. As shown in FIG. 85, XRS 4, XRS 6, XRS 7, XRS 8, and XRS 9 are totally resistant to Exo I digestion. In contrast, the detection of some digestion products indicates that XRS 5 and XRS 10 are only partially resistant.

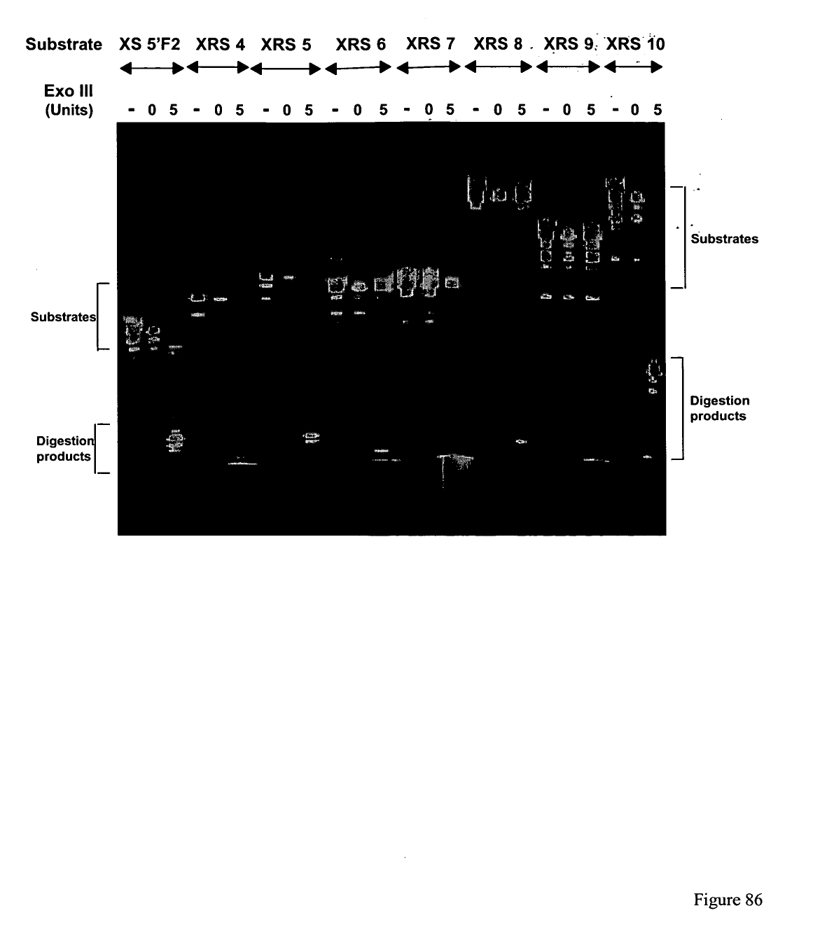

Digestion by Exo III was performed on the same oligos, as shown in FIG. 86. As shown in FIG. 86, XS 5′F2 is degraded by 5 units of Exo III as are XRS 4, XRS 5, and XRS 10. In contrast, XRS 6, XRS 7, XRS 8, and XRS 9 display partial resistance to Exo III digestion.

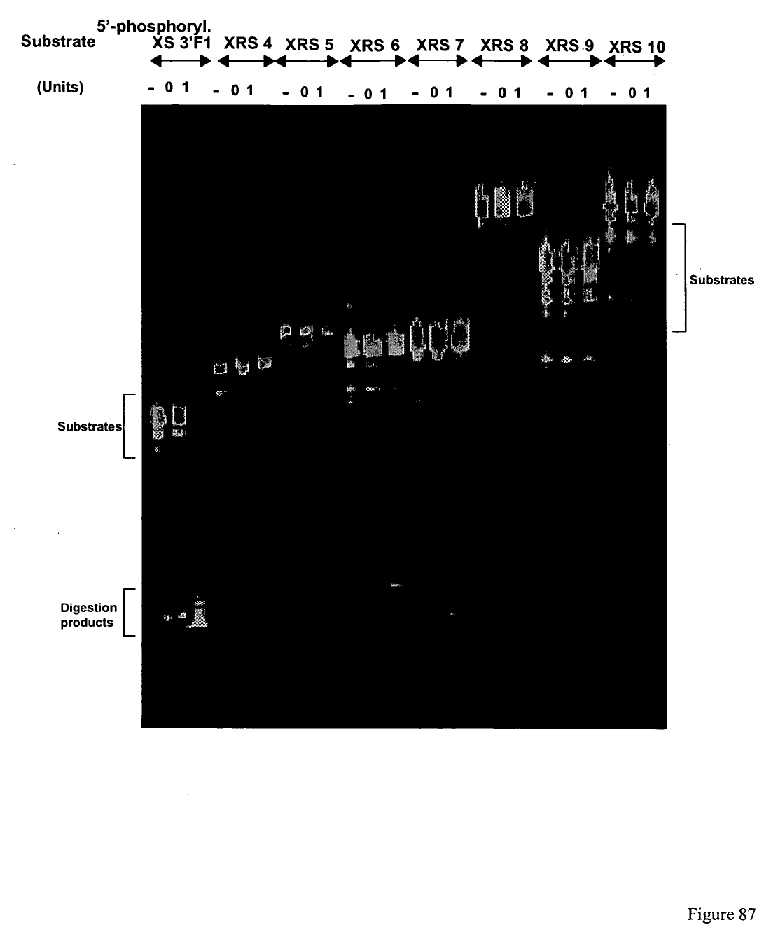

One unit of Lambda exonuclease led to the complete digestion of 5′-phosphorylated XS 3′ F1, shown in FIG. 87, as previously described. As shown in FIG. 87, all the other oligos are resistant to digestion, although XRS 6, XRS 7 and XRS 9 seem very slightly digested, and are therefore considered to be partially resistant.

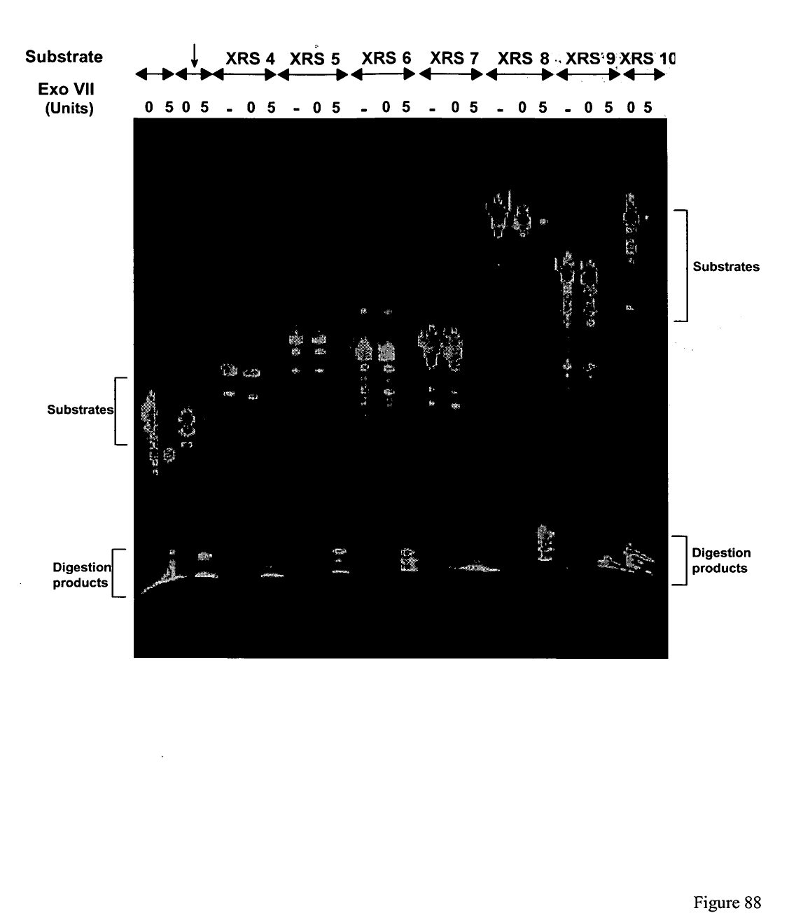

Data obtained with T7 Exonuclease are summarized in Table 6, above. As shown in FIG. 88, none of the tested oligos, including the control oligos, is resistant to Exo VII digestion.

In conclusion, the most effective protecting groups on the 5′-terminus appear to be Biotin-Spacer 18 (on XRS 4) and Biotin-(UOMe)4 (on XRS 8), as well as (UOMe)4-Biotin (on XRS 9). Regarding the 3′-terminus, Spacer 18-Spacer C3, (UOMe)4, Biotin-UOMe, UOMe-Biotin, and (UOMe)4-Biotin and (UOMe)4 are all suitable protecting groups against Exo I digestion. These findings enable the design of LDR oligonucleotide probes with the appropriate blocking groups and thus significantly reduce one important source of background noise.

### Example 12

### Preparation of Q-dot/Q-Zip Conjugates, Immobilization of Biotinylated Oligos on the Streptavidin Microbeads, Hybridization of the Q-Dot/Q-Zip Conjugates, Elution of Q-Dot/Q-Zip Conjugates and Analysis

25 pmol Q-dots were added to 500 pmol double-biotinylated Q-Zips in a final volume of 100 μl, made up with 10 mM HEPES buffer pH7.5. The 20-fold excess of oligonucleotide was used to ensure a high yield of conjugation. Lower loading conditions could be achieved by the use of various ratios of biotinylated oligo to Biocytin. After incubation for 30 minutes at room temperature under gentle agitation, Q-dot/Q-Zip conjugates were purified on Microcon YM-50 spin columns and stored at 4° C. until needed.

0.5 mg of streptavidin-coated microspheres (supplied at 1% suspension in 100 mM borate pH8.5, 0.01% BSA, 0.05% Tween 20, 10 mM EDTA and 0.1% NaN₃) were separated from the storage buffer by magnetic capture and were resuspended in 100 μl TTL buffer (100 mM Tris-HCl pH8, 0.1% Tween 20, 1M LiCl). Magnetic capture was repeated and the microspheres were again resuspended in 50 μl TTL buffer.

0.2 mg aliquots of magnetic beads were incubated for 15 min at room temperature, under gentle agitation, with 68.6 pmol of biotinylated oligodeoxynucleotide to achieve saturation of the microspheres with DNA, in a final volume of 30 μl, made up with TTL buffer. If loading percentages of less than 100% were required, incubation of the microspheres with the appropriate molar quantity of DNA was followed by a second incubation, performed under identical conditions, with 350 pmol Biocytin or a different, unreactive biotinylated oligo (e.g. Zip1), to saturate any excess streptavidin molecules surrounding the spheres.

Following magnetic capture, the microspheres were rinsed twice in TT buffer (250 mM Tris-HCl pH8, 0.1% Tween 20), with appropriate separation steps. The microspheres were then resuspended in 250 μl 2× hybridization buffer (100 mM Tris acetate pH7.5, 600 mM NaCl, 20 mMMgCl₂, 0.1% Tween 20) to give a final concentration of 0.8 μg/μl. Magnetic complexes were stored at 4° C. until needed.

Twelve micrograms (15 μl) of the 0.8 μg/pl magnetic complexes were mixed with 1 pmol of either Q-dot/Q-Zip conjugate, Cy3-labeled control oligo, or 1 pmol water, and the volume was made up to 30 μl with deionized water. This hybridization mixture was incubated at 55° C. for 1 hour in a rotating hybridization oven at position 8. After magnetic capture, magnetic hybrid complexes were washed twice in Wash 1 buffer (50 mM Tris acetate pH7.5, 0.05% Tween 20) at 45° C. for 10 min, and then twice in Wash 2 buffer (50 mM Tris acetate pH7.5, 20 mM NaCl, 0.05% Tween 20) for 10 min at 45° C. Following magnetic capture, magnetic hybrid complexes were resuspended in 20 μl TE buffer.

Two microlitres of 1M NaOH were added to the resuspended magnetic hybrid complexes and incubated at room temperature for 10 min to allow denaturation. The supernatant was collected and neutralized with 0.8 μl 3M sodium acetate pH5.2. Aliquots of 1 μl were loaded onto a glass microscope slide, dried and scanned using GSI Scan Array 5000.

### Example 13

### Plotting of the Q-Dot Standard Curve

0-10 fmol of Q-dots, with no oligonucleotides attached, were spotted directly onto a glass microscope slide. The spots were allowed to dry and the intensity of fluorescence was measured using the ScanArray software on the GSI Lumonics ScanArray 5000 (see Example 1). A standard curve of intensity against quantity of Q-dots, shown in FIG. 90, was plotted using Microsoft Excel. This standard curve allows the quantification of results. Because a saturating signal is obtained from concentrations of Q-dots exceeding 6 fmol, dilutions are sometimes required for accurate quantification.

### Example 14

### Effect of Q-Dot Loading on Signal Intensity

Q-dot/Q-Zip conjugates were prepared using various ratios of oligonucleotide to biocytin (1:0, 1:0.5, 1:1, 1:3, 1:6.6 and 1:13), for both Q-Zip4 and Q-Zip1, as described in Example 12. These Q-dot/Q-Zip conjugates were hybridized onto magnetic microspheres that had oligonucleotide BcQz122 attached at 25% of the total loading capacity of the spheres in a rotating hybridization oven. To allow comparison, Cy3-labeled oligos and Q-dots with Q-Zip1 and Q-Zip2 conjugated at 100% of the loading capacity of the Q-dots. See FIG. 91.

The different loading density of the Q-dots produced signals of variable intensity. The most intense Q-dot/Q-Zip1 signal was obtained from a 1:3 ratio of Q-Zip1 to biocytin (36-fold higher than the signal from the corresponding Q-dot/Q-Zip4 negative control). The intensity of the signal was comparable to that obtained from 100% loading of the Q-dots (27-fold higher than the corresponding Q-dot/Q-Zip4 conjugate signal). Visibly weaker signals were produced from Q-dots with 1:6.6 and 1:13 loading of Q-Zip1 to biocytin. The Cy3-labeled positive control (Cy3Qz1) produced a strong signal.

The loading ratio of the Q-dots does not have a large effect on the efficiency of hybridization, with similar intensity signal produced from Q-dots conjugated to Q-Zip1 to biocytin in ratios of 1:0, 1:0.5, 1: 1, and 1:3. This result is consistent with the Q-dots possessing more than one oligonucleotide, on average, when loading levels greater than this ratio are used. At lower loading levels, each Q-dot may have fewer than one oligonucleotide attached, on average, hence the decrease in the intensity of the signal at 1:6.6 and 1:13 Q-Zip1 to biocytin ratios. Therefore, a 1:3 ratio was used in subsequent Examples.

### Example 15

### Titration of Oligonucleotide Concentration on Magnetic Microspheres Using Biocytin

Variable amounts of oligonucleotide BcQz122 were attached to streptavidin-coated magnetic microspheres giving loading of 100%, 50%, 25%, 10% and 0%. This was followed by saturation of the remaining free streptavidin molecules using an excess of biocytin, as described in Example 12. Cy3-labeled and Q-dot conjugated Q-Zips were incubated with these magnetic conjugates in a rotating hybridization oven. Following denaturation to separate them from the magnetic microspheres, the fluorescent oligos were spotted onto a glass microscope slide and the fluorescent signal was detected using the ScanArray software on the GSI Lumonics ScanArray 5000 (see Example 1). FIG. 92 shows a diagram of Example 15, along with the scanned slide.

The Cy3 labeled Q-Zip1 gave strong signals when hybridized to magnetic beads with attached oligo, although in FIG. 92 the filter sets used are optimized for orange Q-dots, rather than for Cy3 (row A). As shown in FIG. 92, Cy3 labeled p16Ex1 gave virtually no signal (row B), as did magnetic beads with no oligo attached. For Q-dots with no oligo attached (row C) and Q-dot/Q-Zip4 (row D), the intensity of the background signal increases as the amount of oligo on the magnetic beads decreases. For Q-dot/Q-Zip1 (row E) and Q-dot/Q-Zip2 (row F), the signal is strongest for 100% loading of the beads. The signal decreases as the amount of oligonucleotide on the magnetic beads decreases.

The most intense signal is obtained when the magnetic microspheres are saturated with oligonucleotide, at 100% of their loading capacity. Some background signal appears to be produced by non-specific binding when biocytin is used on magnetic beads, and as the amount of BcQz122 surrounding the spheres decreases, the intensity of this background signal increases.

### Example 16

### Titration of Oligonucleotide Concentration on Magnetic Microspheres Using Zip1

Variable amounts of oligonucleotide BcQz122 were attached to streptavidin-coated magnetic microspheres giving loading of 100%, 10%, 1%, 0.1% and 0%. This was followed by saturation of the remaining free streptavidin molecules using an excess of Zip1, as described in Example 11. Cy3-labeled and Q-dot conjugated Q-Zips were incubated with these magnetic conjugates in a rotating hybridization oven. Following denaturation to separate them from the magnetic microspheres, the fluorescent oligos were spotted onto a glass microscope slide and the fluorescent signal was detected using the ScanArray software on the GSI Lumonics ScanArray 5000 (see Example 1). FIG. 93 shows a diagram of Example 16, along with the scanned slide.

The Cy3 labeled Q-Zip1 gave strong signals when hybridized to magnetic beads with attached oligo, although in FIG. 93 the filter sets used are optimized for orange Q-dots, rather than for Cy3 (row A). As shown in FIG. 93, Cy3 labeled p16Ex1 gave virtually no signal (row B), as did magnetic beads with no oligo attached. For Q-dots with no oligo attached (row C) and Q-dot/Q-Zip4 (row D), compared with the analogous positions in FIG. 92, the intensity of the background signal is low, indicating no non-specific binding of either Q-dots or oligos to the magnetic microspheres, and no longer increases as the amount of oligo on the magnetic beads decreases. For Q-dot/Q-Zip1 (row E) and Q-dot/Q-Zip2 (row F), the signal decreases as the amount of oligonucleotide attached to the magnetic beads decreases, but a positive signal is still visible in column C, representing a 100-fold dilution of oligo.

The use of Zip1 to block free streptavidin molecules on the magnetic microspheres results in a lower background signal compared to the use of biocytin as a blocking agent. This would suggest that the biocytin molecules themselves have the ability to bind oligonucleotides and, to a lesser extent, Q-dots, in a non-specific manner. The use of an ‘inert’ biotinylated oligonucleotide circumvents this problem. As shown in FIG. 93, the intensity of signals corresponding to Q-Zip2 (row F) is higher than signals corresponding to Q-Zip1 (row E). This is consistent with the inclusion of two cQ-Zip 2 sequences in B1cQz122 whereas there is only one cQ-Zip 1 sequence, thus allowing twice as many Q-Zip2 Q-dots to hybridize compared to Q-Zip1 Q-dots.

### Example 17

### Estimation of the Relative Intensity of Q-Dot/Q-Zip1 and Q-Dot/Q-Zip2 signals

The products of hybridizations from Example 16 corresponding to FIG. 93 (rows E, F), 100% and 10% loading, were mixed with water to produce a dilution series, shown in FIG. 94, and were spotted onto a microscope slide. The intensity of the fluorescence was measured using the ScanArray software on the GSI Lumonics ScanArray 5000 (see Example 1), and the corresponding quantity of Q-dots was calculated by reference to the standard curve shown in FIG. 90. From this, it was possible to estimate the amount of Q-dots producing the signals.

As shown in FIG. 94, the amount of Q-Zip2 Q-dots corresponding to the intensity of the spot is twice that of Q-Zip1 Q-dots. For the more intense signals, dilution of Q-Zip2 samples with an equal volume of water produces a signal intensity that is comparable to that of Q-Zip1, indicating that the Q-Zip2 signal is roughly twice that of Q-Zip1. The signal intensity of Q-dot/Q-Zip2 may be twice that of Q-dot/Q-Zip1 because of the presence of two cQ-Zip sequences on the magnetic beads compared with one cQ-Zip 1.

### Example 18

### Indirect Hybridization of Q-Dots to Magnetic Microspheres Using a ‘Bridging Oligo’

Biotinylated oligonucleotide Zip1 was attached to magnetic microspheres at 100% of the loading capacity, as described in Example 12. An equimolar amount of bridging oligonucleotide, cZ1 cQ1cQ2, was incubated with the magnetic conjugates at 55° C. in 1× hybridization buffer, in a rotating hybridization oven for 1 hour. After magnetic capture of the hybridized conjugates, samples were washed in Wash buffer 1 and Wash buffer 2 once at 45° C. for 10 minutes, and were resuspended in 250 μl 2× hybridization buffer. A second hybridization was then performed, this time to anneal Cy3-labeled oligonucleotides and Q-dots/Q-Zips onto the bridging oligo, using the conditions described in Example 12. Following denaturation to separate them from the magnetic microspheres, the fluorescent oligos were spotted onto a glass microscope slide and the fluorescent signal was detected using the ScanArray software on the GSI Lumonics ScanArray 5000. FIG. 95 shows a diagram of Example 17, along with the scanned slide. To enable comparison, the results of a direct hybridization of labeled oligos to BcQz122 are shown alongside a negative control, consisting of magnetic microspheres with biotinylated Zip 1 attached, as described in Example 16.

The magnetic microspheres with biotinylated Zip1 attached gave very weak signals for all hybridizations. The Cy3 labeled Q-Zip1 gave strong signals when hybridized to magnetic beads with attached oligo, although in FIG. 95 the filter sets used are optimized for orange Q-dots, rather than for Cy3 (row A). As shown in FIG. 95, Cy3 labeled p16Ex1 gave virtually no signal (row B), as did magnetic beads with no oligo attached. For Q-dots with no oligo attached (row C) and Q-dot/Q-Zip4 (row D), the intensity of the background signal is low, indicating no non-specific binding of either Q-dots or oligos to the magnetic microspheres. For Q-dot/Q-Zip1 (row E) and Q-dot/Q-Zip2 (row F), a strong signal is visible for both the bridging oligo and the direct hybridization. The intensity of the signals produced by direct hybridization (corresponding to 9.8 fmol and 10.0 fmol of Q-dots respectively) are more intense than those using a bridging oligo (corresponding to 6.7 fmol and 6.3 fmol of Q-dots respectively), because in the latter case, a second hybridization was required.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method for identifying one or more target nucleic acid molecules differing by one or more single-base changes, insertions, deletions, or translocations, said method comprising:
providing a test sample potentially containing one or more target nucleic acid molecules differing by one or more single-base changes, insertions, deletions, or translocations;
providing one or more primary oligonucleotide probe sets, each set characterized by (a) a first oligonucleotide probe, having a target-specific portion and (b) a second oligonucleotide probe, having a target-specific portion, wherein the oligonucleotide probes in a particular set are suitable for ligation together when hybridized on a corresponding target nucleic acid molecule, but have a mismatch which interferes with such ligation when hybridized to any other nucleic acid molecule present in the sample, and wherein one or both oligonucleotide probes in the set contain one or more detection oligonucleotide probe-specific portions or their complements such that each probe set contains a unique set of one or more detection oligonucleotide probe-specific portions or their complements;
providing a ligase;
blending the sample, the one or more primary oligonucleotide probe sets, and the ligase to form a primary ligase detection reaction mixture;
subjecting the primary ligase detection reaction mixture to one or more ligase detection reaction cycles comprising a denaturation treatment, wherein any hybridized oligonucleotides are separated from the target nucleic acid molecules, and a hybridization treatment, wherein the primary oligonucleotide probe sets hybridize in a base-specific manner to their respective target nucleic acid molecules, if present in the sample, and ligate to one another to form a primary ligation product containing the target-specific portions and one or more detection oligonucleotide probe-specific portions or their complements, with the primary ligation product for each of the primary oligonucleotide probe sets being distinguishable from other nucleic acid molecules in the primary ligase detection reaction mixture by a unique set of one or more detection oligonucleotide probe-specific portions or their complements, and, wherein the primary oligonucleotide probe sets may hybridize to nucleic acid molecules in the sample other than their respective target nucleic acid molecules but do not ligate together due to a presence of one or more mismatches and individually separate during the denaturation treatment;
providing detection oligonucleotide probes which bind to the complementary detection oligonucleotide probe-specific portion of the primary ligation product or complements thereof, wherein each detection oligonucleotide probe has a reporter label, thereby providing each primary ligation product with a unique detectable encryption code;
contacting the primary ligation products and the detection oligonucleotide probes under conditions effective to permit hybridization of the detection oligonucleotide probes to the primary ligation products so that a labeled primary ligation product is formed; and
detecting the reporter label(s) on the primary ligation product, thereby indicating the presence of one or more target nucleic acid molecules in the sample, wherein nucleic acid molecules differing by one or more single-base changes, insertions, deletions, or translocations are discriminated from one another during the one or more ligase detection reaction cycles and the discriminated nucleic acid molecules are detected as a result of each different labeled, primary ligation product having a unique encryption code with a different pattern of detectable emission spectra.

2. The method according to claim 1 further comprising:
capturing the individual primary ligation products or complements thereof on one or a plurality of solid supports after said subjecting the primary ligase detection reaction mixture to one or more ligase detection reaction cycles and prior to said contacting the captured primary ligation products and the detection oligonucleotide probes, whereby each primary ligation product is individually distinguished.

3. The method according to claim 2, wherein relative amounts of one or more of a plurality of target nucleic acid molecules in the test sample, differing by one or more single-base changes, insertions, deletions, or translocations, are quantified by comparison with a reference sample having reference target nucleic acid molecules, said method further comprising:
providing one or more secondary oligonucleotide probe sets, which differ from the primary oligonucleotide probe sets, wherein each of the secondary oligonucleotide probes sets are characterized by (a) a first oligonucleotide probe having a reference target-specific portion and (b) a second oligonucleotide probe having a reference target-specific portion, wherein the oligonucleotide probes in a particular secondary oligonucleotide probe set are suitable for ligation together when hybridized adjacent to a corresponding reference target nucleic acid molecule, but have a mismatch which interferes with such ligation when hybridized to any other nucleic acid molecule present in the reference sample, and wherein one or both oligonucleotide probes in the secondary oligonucleotide probe set contain one or more detection oligonucleotide probe-specific portions or their complements such that each secondary oligonucleotide probe set contains a unique set of one or more detection oligonucleotide probe-specific portions or their complements;
blending the reference sample, the one or more secondary oligonucleotide probe sets, and the ligase to form a secondary ligase detection reaction mixture;
subjecting the secondary ligase detection reaction mixture to one or more ligase detection reaction cycles comprising a denaturation treatment, wherein any hybridized oligonucleotides are separated from reference target nucleic acid molecules, and a hybridization treatment, wherein the oligonucleotide probe sets hybridize in a base-specific manner to their respective reference sample target nucleic acid molecule, if present in the sample, and ligate to one another to form a secondary ligation product containing (a) the reference sample-specific portions and (b) the one or more detection oligonucleotide probe-specific portions or their complements with the secondary ligation product for each secondary oligonucleotide probe set being distinguishable from other nucleic acid molecules in the secondary ligase detection reaction mixture, and, wherein the secondary oligonucleotide probe sets may hybridize to nucleic acid molecules in the reference sample other than their respective reference target nucleic acid molecules but do not ligate together due to a presence of one or more mismatches and individually separate during the denaturation treatment;
blending the first and second ligase detection reaction mixtures after subjecting them to one or more ligase detection reaction cycles and before said capturing, whereby the blended first and second ligase detection reaction mixtures are subjected to said capturing, said contacting, and said detecting; and
comparing relative amounts of the reporter labels on the primary and secondary ligation products, to provide a quantitative measure of the relative level of the one or more target nucleic acid molecules in the test sample compared with the reference sample based on each different labeled ligation product having a unique encryption code with a different pattern of detectable emission spectra.

4. The method according to claim 2 further comprising:
subjecting the primary ligation detection reaction mixture to exonuclease digestion after said subjecting the primary ligase detection reaction mixture to one or more ligase detection reaction cycles and prior to said capturing under conditions effective to destroy unligated oligonucleotide probes.

5. The method according to claim 4, wherein for each primary oligonucleotide probe set, the first oligonucleotide probe contains a blocking group on its 5′ end, rendering the first oligonucleotide probe resistant to a 5′→3′exonuclease, and the second oligonucleotide probe contains a blocking group on its 3′ end, rendering it resistant to a 3′→5′exonuclease.

6. A method according to claim 5 further comprising:
filtering to remove capture agents from the primary ligation detection reaction mixture after exonuclease digestion.

7. The method according to claim 2, wherein one of the oligonucleotide probes in the oligonucleotide probe set contains one or more detection oligonucleotide probe-specific portions or their complements.

8. The method according to claim 2, wherein both of the oligonucleotide probes in the oligonucleotide probe set contains one or more detection oligonucleotide probe-specific portions or their complements.

9. The method according to claim 2, wherein the first oligonucleotide probe has a binding agent which is incorporated in any primary ligation product, and the solid support has one or more attached binding partner to the binding agent, whereby said capturing is carried out under conditions effective for the binding agent and its binding partner to become coupled together, thereby immobilizing any primary ligation product to the solid support.

10. The method according to claim 9, wherein the binding agent-binding partner pairs are selected from the group consisting of antibody-antigen binding partners, streptavidin-biotin binding partners, complementary oligonucleotides, amino group and EDC activated carboxylic acid group, thiol based binding partners, histidine moieties and nickel-NTA, and other chemical moieties that may be covalently or ionically linked to each other.

11. The method according to claim 2, wherein the solid support is a paramagnetic bead and said method further comprises:
recovering the paramagnetic beads by magnetic attraction after said capturing and placing the recovered paramagnetic beads on a microscope slide.

12. The method according to claim 2, wherein a complement of the primary ligation product sequence is captured on the solid support, said method further comprising:
subjecting the primary ligation product to a polymerase extension reaction after said subjecting the primary ligase detection reaction mixture to one or more ligase detection reaction cycles and prior to said capturing.

13. The method according to claim 2, wherein the primary ligation product is captured on the solid support.

14. The method according to claim 2, wherein the reporter label(s) are nanocrystals and said detecting comprises:
exciting the nanocrystals to produce an emission spectrum and
evaluating the emission spectra of the nanocrystals.

15. The method according to claim 2, wherein the ligase is selected from the group consisting of Thermus aquaticus ligase, Thermus thermophilus ligase, AK16D thermostable ligase, E. coli ligase, T4 ligase, and Pyrococcus ligase.

16. The method according to claim 2, wherein the target-specific portions of the oligonucleotide probes each have a hybridization temperature of 20-85° C.

17. The method according to claim 2, wherein the target-specific portions of the oligonucleotide probes are 15 to 30 nucleotides long.

18. The method according to claim 2, wherein the oligonucleotide probe sets are selected from the group consisting of ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, and mixtures thereof.

19. The method according to claim 2, wherein said method is used to detect infectious diseases caused by bacterial, viral, parasitic, and fungal infectious agents.

20. The method according to claim 19, wherein the infectious disease is caused by a bacterium selected from the group consisting of Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Actinomycetes.

21. The method according to claim 19, wherein the infectious disease is caused by a fungal infectious agent selected from the group consisting of Cryptococcus neoformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis.

22. The method according to claim 19, wherein the infectious disease is caused by a viral infectious agent selected from the group consisting of human immunodeficiency virus, human T-cell lymphocytotrophic virus, hepatitis viruses, Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

23. The method according to claim 19, wherein the infectious disease is caused by a parasitic infectious agent selected from the group consisting of Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

24. The method according to claim 2, wherein said method is used to detect genetic diseases.

25. The method according to claim 24, wherein the genetic disease is selected from the group consisting of 21 hydroxylase deficiency, cystic fibrosis, Fragile X Syndrome, Turner Syndrome, Duchenne Muscular Dystrophy, Down Syndrome, heart disease, single gene diseases, HLA typing, phenylketonuria, sickle cell anemia, Tay-Sachs Syndrome, thalassemia, Klinefelter's Syndrome, Huntington's Disease, autoimmune diseases, lipidosis, obesity defects, hemophilia, inborn errors in metabolism, and diabetes.

26. The method according to claim 2, wherein said method is used to detect cancer involving oncogenes, tumor suppressor genes, or genes involved in DNA amplification, replication, recombination, or repair, or SNPs or adjacent regions that serve as markers for copy number changes or loss of heterozygosity in such genes.

27. The method according to claim 26, wherein the cancer is associated with a gene selected from the group consisting of APC, AKT, ALT, AXL, BAX, Bcl2, Beta-Catenin, bFGF, BRCA1, BRCA2, Braf, Cdc25A, Cdk4, c-Fos, c-Jun, c-Kit, C-met, c-Myc, c-Ret, CSF1R, CSF2, c-Src, CYCD-CDK4, CYCE-CDK2, Cyclin D1, Cyclin E1, Cytokines, Dishevelled, E2F, E-Cadherin, EGFR, elF4E, ErbB-3, ErbB-4, FGFR-1, FGFR-2, FGFR-3, FGFR-4, FH4 (VEGFR-3), Fit-1 (VEGFR-1), Flk-1 (VEGFR-2), Frizzled, G Proteins, GPCR, GRB2-SOS, GSK3 beta, Her2-neu, HGF, HSP27, HSP70, IFGII, IGFR1, K-ras, H-ras, N-ras, LT, MAPK, MDM2, MEK, MLH1, MSH2, MSH6, MYC, p15^{INK4b}, p16^{INK4a}, p19^{ARF}, p21^{Cip}, p27^{Kip}, p53, PDGFR alpha, PDGFR beta, PI3K, PP2A, PTEN, RAF, RAS, RB, Ron, RSK, RTK, Ski, Smad2, Smad4, ST, surviving, TbRII, TCF, Tcf4, TERT, TGF-Beta, TGF-Beta R, TIC2, TOR, VEGF, WAF1, Wisp-1, Wisp-3, WNT, or SNPs or adjacent regions that serve as markers for copy number changes or loss of heterozygosity in such genes, human papillomavirus Types 16 and 18, leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, ENT tumors, and loss of heterozygosity.

28. The method according to claim 2, wherein said method is used for environmental monitoring, forensics, and food and feed industry monitoring.

29. The method according to claim 2, wherein a plurality of primary oligonucleotide probe sets are utilized with each set characterized by (a) the first oligonucleotide probe being identical in each oligonucleotide probe set and (b) the second oligonucleotide probes in each set having a target-specific portion which is different in each second oligonucleotide probe at a location where single-base changes, insertions, deletions, or translocations occur.

30. The method according to claim 2, wherein the target-specific portions of the primary oligonucleotide probe sets have a 3′ discriminating base.

31. The method according to claim 1, wherein the primary ligase detection reaction mixture is subjected to one ligase detection reaction cycle.

32. The method according to claim 1, wherein one or both of the oligonucleotide probes in the primary oligonucleotide probe set contain a plurality of detection oligonucleotide probe-specific portions or their complements.

33. The method according to claim 1, wherein the detection oligonucleotide probe-specific portions of the primary ligation products or complements thereof is selected from the set of sequences shown in FIGS. 97, 98, 99A, 100A, 101A, 102A, 103A, 104A, 105A, 106A, and or 107A or their complements.

34. The method according to claim 2, wherein the relative amounts of two or of a plurality of nucleic acid molecules, differing by one or more single-base changes, insertions, deletions, or translocations and present in a sample in unknown amounts with a plurality of target nucleic acid molecules are quantified, said method further comprising;
quantifying the relative amount of the ligation products after said detecting and
comparing the relative amounts of the ligation products to provide a quantitative measure of the relative amounts of two or a plurality of target nucleic acid molecules in the sample.

35. The method according to claim 34, wherein within one or more of the ligase detection reaction cycles includes internal cycles comprising a hybridization treatment, wherein the oligonucleotide probe sets hybridize at adjacent positions in a base-specific manner to their respective target nucleic acid molecules, if present in the sample, and ligate to one another to form a ligation product and a probe denaturation treatment, wherein when the primary ligase detection reaction mixture is heated to a temperature above that at which each target-specific portion melts, unligated probes separate from the nucleic acid molecules to which they are hybridized, and when heated to a temperature below the melting temperature of each target-specific portion, ligation products hybridized to nucleic acid molecules accumulate with each successive internal cycle to provide a quantitative measure of the relative level of two or more target nucleic acid molecules in the sample.

36. A method for identifying one or more target nucleic acid molecules differing by one or more single-base changes, insertions, deletions, or translocations, said method comprising:
providing a test sample potentially containing one or more target nucleic acid molecules;
providing one or more primary oligonucleotide probe sets, each set characterized by (a) a first oligonucleotide probe, having a target-specific portion and a 5′ upstream portion containing a translational oligonucleotide portion and (b) a second oligonucleotide probe, having a target-specific portion, and a 3′ downstream primer-specific portion, wherein the oligonucleotide probes in a particular primary oligonucleotide probe set are suitable for ligation together when hybridized to a corresponding target nucleic acid molecule, but have a mismatch which interferes with such ligation when hybridized to any other nucleic acid molecule present in the test sample;
providing a ligase;
blending the test sample, the one or more primary oligonucleotide probe sets, and the ligase to form a primary ligase detection reaction mixture;
subjecting the primary ligase detection reaction mixture to one or more ligase detection reaction cycles comprising a denaturation treatment, wherein any hybridized oligonucleotide probes are separated from the target nucleic acid molecules, and a hybridization treatment, wherein the primary oligonucleotide probe sets hybridize in a base-specific manner to their respective target nucleic acid molecules, if present in the test sample, and ligate to one another to form a primary ligation product containing (a) the 5′ upstream translational oligonucleotide portion, (b) the target-specific portions, and (c) the 3′ downstream primer-specific portion, with the primary ligation product for each primary oligonucleotide probe set being distinguishable from other nucleic acids in the ligase detection reaction mixture, and, wherein the primary oligonucleotide probe sets may hybridize to nucleic acid molecules in the test sample other than their respective target nucleic acid molecules but do not ligate together due to a presence of one or more mismatches and individually separate during the denaturation treatment;
providing a downstream primer complementary to the 3′ downstream primer-specific portion of the primary ligation product;
providing a polymerase;
blending the primary ligation product with the downstream primer, and the polymerase to form a polymerase extension reaction mixture;
subjecting the polymerase extension reaction mixture to one or more polymerase chain reaction cycles comprising a denaturation treatment, wherein hybridized nucleic acid molecules are separated, a hybridization treatment, wherein the primer hybridizes to its complementary 3′ downstream primer-specific portion of the primary ligation product, and an extension treatment, wherein the hybridized primers are extended to form extension products complementary to the primary ligation product;
capturing the extension product on one or more solid supports, so that the extension product may be individually distinguished,
providing one or more secondary oligonucleotide probe sets, each set characterized by (a) a first oligonucleotide probe, having a translational oligonucleotide portion and a 5′ upstream portion complementary to one or more detection oligonucleotide probe-specific portions and (b) a second oligonucleotide probe, having a target portion, and a 3′ downstream portion complementary to one or more detection oligonucleotide probe-specific portions, wherein the oligonucleotide probes in a particular secondary oligonucleotide probe set are suitable for ligation together when hybridized to a corresponding captured primary extension product, but have a mismatch which interferes with such ligation when hybridized to any other nucleic acid molecule;
blending the captured extension product, the one or more secondary oligonucleotide probe sets, and the ligase to form a second ligase detection reaction mixture;
subjecting the second ligase detection reaction mixture to one ligase detection reaction cycle comprising a denaturation treatment, wherein any hybridized oligonucleotides are separated from the captured extension product, and a hybridization treatment, wherein the secondary oligonucleotide probe sets hybridize in a base-specific manner to their respective captured extension products, if present, and ligate to one another to form a secondary ligation product containing (a) the 5′ upstream portion comprising one or more detection oligonucleotide probe-specific portions, (b) the upstream translational oligonucleotide portion connected to the target portion, and (c) the 3′ downstream portion comprising one or more detection oligonucleotide probe-specific portions, with the secondary ligation product for each secondary oligonucleotide probe set being distinguishable from other nucleic acids in the second ligase detection reaction mixture, and, wherein the one or more secondary oligonucleotide probe sets may hybridize to nucleic acid molecules in the sample other than their respective captured extension products but do not ligate together due to a presence of one or more mismatches and individually separate during the denaturation treatment by heating to above the temperature at which each translational oligonucleotide portion melts, but below a temperature at which each secondary ligation product melts, whereby each secondary ligation product remains hybridized to the captured extension product as a complex;
providing detection oligonucleotide probes which bind to the detection oligonucleotide probe-specific portions of the complex, wherein each detection oligonucleotide probe has a reporter label, thereby providing each complex containing a secondary ligation product with a unique detectable encryption code;
contacting the complex and the detection oligonucleotide probes under conditions effective to permit hybridization of the detection oligonucleotide probes to the complex so that a labeled complex is formed; and
detecting the reporter label(s) on the complex, thereby indicating the presence of one or more target nucleic acid molecule(s) in the test sample, wherein nucleic acid molecules differing by one or more single-base changes, insertions, deletions, or translocations are discriminated from one another during the primary and secondary ligase detection reactions and the discriminated nucleic acid molecules are detected as a result of each different labeled complex having a unique encryption code with a different pattern of detectable emission spectra.

37. The method according to claim 35, wherein relative amounts of one or more of a plurality of target nucleic acid molecules in the test sample, differing by one or more single-base changes, insertions, deletions, or translocations, are quantified by comparison with a reference sample, said method comprising:
providing one or more tertiary oligonucleotide probe sets, which differ from the primary oligonucleotide probe sets, wherein each of the tertiary oligonucleotide probes sets are characterized by (a) a first oligonucleotide probe, having a reference target-specific portion and a 5′ upstream portion containing a translational oligonucleotide probe-specific portion and (b) a second oligonucleotide probe, having a reference target-specific portion, and a 3′ downstream primer specific portion, wherein the oligonucleotide probes in a particular tertiary oligonucleotide probe set are suitable for ligation together when hybridized on a corresponding reference target nucleic acid molecule, but have a mismatch which interferes with such ligation when hybridized to any other nucleic acid molecule present in the reference sample;
blending the reference sample, the one or more tertiary oligonucleotide probe sets, and the ligase to form a tertiary ligase detection reaction mixture;
subjecting the tertiary ligase detection reaction mixture to one or more ligase detection reaction cycles comprising a denaturation treatment, wherein any hybridized oligonucleotides are separated from the reference target nucleic acid molecules, and a hybridization treatment, wherein the oligonucleotide probe sets hybridize in a base-specific manner to their respective reference target nucleic acid molecule, if present in the sample, and ligate to one another to form a tertiary ligation product containing (a) the 5′ upstream translational oligonucleotide-specific portion, (b) the target-specific portions, and (c) the 3′ downstream primer specific portion with the tertiary ligation product for each tertiary oligonucleotide probe set being distinguishable from other nucleic acid molecules in the tertiary ligase detection reaction mixture, and, wherein the tertiary oligonucleotide probe sets may hybridize to nucleic acid molecules in the sample other than their respective reference target nucleic acid molecules but do not ligate together due to a presence of one or more mismatches and individually separate during the denaturation treatment;
blending the primary and tertiary ligase detection reaction mixtures after subjecting them to one or more ligase detection reaction cycles and before said subjecting the polymerase chain reaction mixture to one or more polymerase extension reaction cycles, whereby the blended primary and tertiary ligase detection reaction mixtures are subjected to one or more polymerase chain reaction cycles, said capturing, said subjecting the secondary ligase detection reaction mixture to one ligase detection reaction cycles, said contacting the complex and the detection oligonucleotide probes, and said detecting the reporter labels on the complex; and
comparing relative amounts of the reporter label on the complexes, to provide a quantitative measure of the relative level of the one or more target nucleic acid molecules in the test sample compared with the reference sample as a result of each different labeled complex having a unique encryption code with a different pattern of detectable emission spectra.

38. The method according to claim 36 further comprising:
subjecting the primary ligation detection reaction mixture to exonuclease digestion after said subjecting the primary ligase detection reaction mixture to one or more ligase detection reaction cycles and prior to said capturing under conditions effective to destroy unligated oligonucleotide primers.

39. The method according to claim 38, wherein for each primary oligonucleotide probe set, the first oligonucleotide probe contains a blocking group on its 5′ end, rendering the first oligonucleotide probe resistant to a 5′→3′exonuclease, and the second oligonucleotide probe contains a blocking group on its 3′ end, rendering it resistant to a 3′→5′exonuclease.

40. A method according to claim 38 further comprising:
filtering to remove capture agents from the primary ligation detection reaction mixture after exonuclease digestion.

41. The method according to claim 36, wherein both of the oligonucleotide probes in each of the secondary oligonucleotide probe sets contains one or more detection oligonucleotide probe-specific portions.

42. The method according to claim 36, wherein the first oligonucleotide probe of each of the primary oligonucleotide probe sets has a binding agent which is incorporated in any primary ligation product, and the solid support has one or more attached binding partner to the binding agent, whereby said capturing is carried out under conditions effective for the binding agent and its binding partner to become coupled together, thereby immobilizing any primary ligation product to the solid support.

43. The method according to claim 42, wherein the binding agent-binding partner pairs are selected from the group consisting of antibody-antigen binding partners, streptavidin-biotin binding partners, complementary oligonucleotides, amino group and EDC activated carboxylic acid group, thiol based binding partners, histidine moieties and nickel-NTA, and other chemical moieties that may be covalently or ionically linked to each other.

44. The method according to claim 36, wherein the solid support is a paramagnetic bead and said method further comprises:
recovering the paramagnetic beads by magnetic attraction after said capturing and
placing the recovered paramagnetic beads on a slide.

45. The method according to claim 36, wherein a complement of the primary ligation product sequence is captured on the solid support.

46. The method according to claim 36, wherein the reporter labels are nanocrystals and said detecting comprises:
exciting the nanocrystals to produce emission spectra and
evaluating the emission spectra of the nanocrystals.

47. The method according to claim 36, wherein the ligase is selected from the group consisting of Thermus aquaticus ligase, Thermus thermophilus ligase, AK16D thermostable DNA ligase, E. coli ligase, T4 ligase, and Pyrococcus ligase.

48. The method according to claim 36, wherein the target-specific portions of the oligonucleotide probes each have a hybridization temperature of 20-85° C.

49. The method according to claim 36, wherein the target-specific portions of the oligonucleotide probes are 15 to 30 nucleotides long.

50. The method according to claim 36, wherein the oligonucleotide probe sets are selected from the group consisting of ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, and mixtures thereof.

51. The method according to claim 36, wherein said method is used to detect infectious diseases caused by bacterial, viral, parasitic, and fungal infectious agents.

52. The method according to claim 51, wherein the infectious disease is caused by a bacterium selected from the group consisting of Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Actinomycetes.

53. The method according to claim 51, wherein the infectious disease is caused by a fungal infectious agent selected from the group consisting of Cryptococcus neoformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis.

54. The method according to claim 51, wherein the infectious disease is caused by a viral infectious agent selected from the group consisting of human immunodeficiency virus, human T-cell lymphocytotrophic virus, hepatitis viruses, Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

55. The method according to claim 51, wherein the infectious disease is caused by a parasitic infectious agent selected from the group consisting of Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

56. The method according to claim 36, wherein said method is used to detect genetic diseases.

57. The method according to claim 56, wherein the genetic disease is selected from the group consisting of 21 hydroxylase deficiency, cystic fibrosis, Fragile X Syndrome, Turner Syndrome, Duchenne Muscular Dystrophy, Down Syndrome, heart disease, single gene diseases, HLA typing, phenylketonuria, sickle cell anemia, Tay-Sachs Syndrome, thalassemia, Klinefelter's Syndrome, Huntington's Disease, autoimmune diseases, lipidosis, obesity defects, hemophilia, inborn errors in metabolism, and diabetes.

58. The method according to claim 36, wherein said method is used to detect cancer involving oncogenes, tumor suppressor genes, or genes involved in DNA amplification, replication, recombination, or repair, or SNPs or adjacent regions that serve as markers for copy number changes or loss of heterozygosity in such genes.

59. The method according to claim 58, wherein the cancer is associated with a gene selected from the group consisting of APC, AKT, ALT, AXL, BAX, Bcl2, Beta-Catenin, bFGF, BRCA1, BRCA2, Braf, Cdc25A, Cdk4, c-Fos, c-Jun, c-Kit, C-met, c-Myc, c-Ret, CSF1R, CSF2, c-Src, CYCD-CDK4, CYCE-CDK2, Cyclin D1, Cyclin E1, Cytokines, Dishevelled, E2F, E-Cadherin, EGFR, elF4E, ErbB-3, ErbB-4, FGFR-1, FGFR-2, FGFR-3, FGFR-4, FH4 (VEGFR-3), Fit-1 (VEGFR-1), Flk-1 (VEGFR-2), Frizzled, G Proteins, GPCR, GRB2-SOS, GSK3 beta, Her2-neu, HGF, HSP27, HSP70, IFGII, IGFR1, K-ras, H-ras, N-ras, LT, MAPK, MDM2, MEK, MLH1, MSH2, MSH6, MYC, p15^{INK4b}, p16^{Ink4a}, p19^{ARF}, p21^{Cip}, p27^{Kip}, p53, PDGFR alpha, PDGFR beta, PI3K, PP2A, PTEN, RAF, RAS, RB, Ron, RSK, RTK, Ski, Smad2, Smad4, ST, surviving, TbRII, TCF, Tcf4, TERT, TGF-Beta, TGF-Beta R, TIC2, TOR, VEGF, WAF1, Wisp-1, Wisp-3, WNT, or SNPs or adjacent regions that serve as markers for copy number changes or loss of heterozygosity in such genes, human papillomavirus Types 16 and 18, leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, ENT tumors, and loss of heterozygosity.

60. The method according to claim 36, wherein said method is used for environmental monitoring, forensics, and food and feed industry monitoring.

61. The method according to claim 36, wherein a plurality of primary oligonucleotide probe sets are utilized with each set characterized by (a) the first oligonucleotide probe being identical in each oligonucleotide probe set and (b) the second oligonucleotide probes in each set having a target-specific portion which is different in each second oligonucleotide probe at a location where single-base changes, insertions, deletions, or translocations occur.

62. The method according to claim 36, wherein the primary ligase detection reaction mixture is subject to one ligase detection reaction cycle.

63. The method according to claim 36, wherein one or both of the oligonucleotide probes in each of the secondary oligonucleotide probe sets contain a plurality of detection oligonucleotide probe-specific portions, said method further comprising:
ligating the plurality of detection oligonucleotide probes hybridized to a particular oligonucleotide probe of the secondary oligonucleotide probe set after said contacting the complex and the detection oligonucleotide probes and before said detecting.

64. The method according to claim 37, wherein the 5′ upstream translational oligonucleotide-specific portion is selected from the set of sequences shown in FIGS. 120A and 122A or their complements.

65. The method according to claim 37, wherein the oligonucleotide probes having a translational oligonucleotide portion and a 5′ upstream portion complementary to one or more detection oligonucleotide probe-specific sequences is selected from the set of sequences shown in FIGS. 121A and 123A or their complements.

66. The method according to claim 37, wherein the 3′ downstream primer-specific portion is selected from the set of sequences shown in FIGS. 108A, 109A, 110A, 111A, 112A, and 113A or their complements.

67. The method according to claim 37, wherein the relative amounts of two or of a plurality of sequences, differing by one or more single-base changes, insertions, deletions, or translocations and present in a sample in unknown amounts with a plurality of target nucleic acid molecules are quantified, said method further comprising;
quantifying the relative amount of the ligation products, after said detecting and
comparing the relative amounts of the ligation products to provide a quantitative measure of the relative amounts of two or a plurality of target nucleic acid molecules in the sample.

68. The method according to claim 67, wherein within one or more of the ligase detection reaction cycles includes internal cycles comprising a hybridization treatment, wherein the oligonucleotide probe sets hybridize at adjacent positions in a base-specific manner to their respective target nucleic acid molecules, if present in the sample, and ligate to one another to form a ligation product, and a probe denaturation treatment, wherein, when the reaction mixture is heated to a temperature above that at which each target-specific portion melts, unligated probes separate from the nucleic acid molecules to which they are hybridized and when heated to a temperature below the melting temperature of each target-specific portion, ligation products hybridized to nucleic acid molecules accumulate with each successive internal cycle to provide a quantitative measure of the relative level of two or more target nucleic acid molecules in the sample.

69. The method according to claim 37, wherein the test sample comprises nucleic acid molecules isolated from a tumor, and the reference sample comprises nucleic acid molecules isolated from normal tissue or matched blood, wherein the relative level of the one or more target nucleic acid molecules in the test sample compared with the reference sample provides a measure of allele imbalance in the tumor at the corresponding loci.

70. The method according to claim 36, wherein the target-specific portions of the primary oligonucleotide probe sets have a 3′ discriminating base.

71. A method for identifying one or more target nucleic acid molecules, differing by one or more single-base changes, insertions, deletions, or translocations, in a plurality of nucleic acid molecules or identifying one or more target mRNA molecules differing by one or more splice site variations in a plurality of mRNA molecules, said method comprising:
providing a test sample potentially containing one or more target nucleic acid molecules differing by one or more single-base changes, insertions, deletions, or translocations, in a plurality of nucleic acid molecules or one or more target mRNA molecules differing by one or more splice site variations in a plurality of mRNA molecules;
providing one or more primary oligonucleotide probe sets, each set characterized by (a) a first oligonucleotide probe, having one or more detection oligonucleotide probe-specific portions or their complements and a target-specific portion and (b) a second oligonucleotide probe, having a target-specific portion and an addressable array-specific portion or its complement, wherein the oligonucleotide probes in a particular set are suitable for ligation together when hybridized to a corresponding target nucleic acid molecule or target mRNA molecule, but have a mismatch which interferes with such ligation when hybridized to any other nucleic acid molecule or mRNA molecule present in the sample, and such that each probe set contains a unique combination of detection oligonucleotide probe-specific portions and addressable array-specific portions or their complements;
providing a ligase;
blending the sample, the one or more oligonucleotide probe sets, and the ligase to form a primary ligase detection reaction mixture;
subjecting the primary ligase detection reaction mixture to one or more primary ligase detection reaction cycles comprising a denaturation treatment, wherein any hybridized oligonucleotides are separated from the target nucleic acid molecules or target mRNA molecules, and a hybridization treatment, wherein the one or more oligonucleotide probe sets hybridize in a base-specific manner to their respective target nucleic acid molecules or target mRNA molecules, if present in the sample, and ligate to one another to form a primary ligation product containing (a) the one or more detection oligonucleotide probe-specific portions or their complements, (b) the target-specific portions, and (c) the addressable array-specific portion or its complement, with the primary ligation product sequence for each one or more oligonucleotide probe set being distinguished from other nucleic acid molecules or mRNA molecules in the primary ligase detection reaction mixture by virtue of their containing a unique combination of detection oligonucleotide probe-specific portions and addressable array-specific portions or their complements, and wherein the primary oligonucleotide probe sets may hybridize to nucleic acid molecules or mRNA molecules in the sample other than their respective target nucleic acid molecules or target mRNA molecules but do not ligate together due to a presence of one or more mismatches and individually separate during the denaturation treatment;
providing a solid support with capture oligonucleotide probes immobilized at different sites, wherein the capture oligonucleotide probes have nucleotide sequences complementary to the addressable array-specific portions or their complements;
contacting the primary ligation products, copies of primary ligation products, or complements thereof with the solid support under conditions effective to hybridize the primary ligation products, copies of primary ligation products, or complements thereof to the capture oligonucleotide probes in a base-specific manner, thereby capturing the primary ligation products, copies of primary ligation products, or complements thereof on the solid support at the site with the complementary capture oligonucleotide,
providing detection oligonucleotide probes which bind to the detection oligonucleotide probe-specific portions of the captured primary ligation products, copies of primary ligation products, or complements thereof, wherein each detection oligonucleotide probe has a reporter label, such that each of the captured primary ligation products, copies of primary ligation products, or
complements thereof have a unique detectable encryption code;
contacting the captured primary ligation products, copies of primary ligation products, or complements thereof and the detection oligonucleotide probes under conditions effective to permit hybridization of the detection oligonucleotide probes to the captured primary ligation products, copies of primary ligation products, or complements thereof so that labeled, captured primary ligation products, copies of primary ligation products, or complements thereof are formed; and
detecting the reporter label on the labeled, captured primary ligation products, copies of primary ligation products, or complements thereof, thereby indicating the presence of one or more target nucleic acid molecules or target mRNA molecules in the sample, wherein target nucleic acid molecules differing by one or more single-base changes, insertions, deletions, or translocations in a plurality of nucleic acid molecules or target mRNA molecules differing by one or more splice site variations in a plurality of mRNA molecules are discriminated from one another during the primary ligase detection reaction and the discriminated molecules are detected as a result of different labeled ligation products having encryption codes with a different pattern of detectable emission spectra, at different sites on the solid support.

72. The method according to claim 71, wherein relative amounts of one or more target nucleic acid molecules in the test sample, differing by one or more single-base changes, insertions, deletions, or translocations, or one or more target mRNA molecules differing by one or more splice site variations, are quantified by comparison with a reference sample having reference target nucleic acid molecules or reference target mRNA molecules, said method comprising:
providing one or more secondary oligonucleotide probe sets, which differ from the primary oligonucleotide probe sets, wherein each of the secondary oligonucleotide probes sets are characterized by (a) a first oligonucleotide probe, having one or more detection oligonucleotide probe-specific portions or their complements and a reference target-specific portion, and (b) a second oligonucleotide probe, having a reference target-specific portion and an addressable array-specific portion or its complement, wherein the oligonucleotide probes in a particular secondary oligonucleotide probe set are suitable for ligation together when hybridized to one another on a corresponding reference target nucleic acid molecule or reference target mRNA molecules, but have a mismatch which interferes with such ligation when hybridized to any other nucleic acid or mRNA molecule present in the reference sample, and wherein one or both oligonucleotide probes in the secondary oligonucleotide probe set contain one or more detection oligonucleotide probe-specific portions or their complements such that each secondary oligonucleotide probe set contains a unique set of one or more detection oligonucleotide probe-specific portions or their complements;
blending the reference sample, the one or more secondary oligonucleotide probe sets, and the ligase to form a secondary ligase detection reaction mixture;
subjecting the secondary ligase detection reaction mixture to one or more ligase detection reaction cycles comprising a denaturation treatment, wherein any hybridized oligonucleotides are separated from reference target nucleic acid molecules or reference target mRNA molecules, and a hybridization treatment, wherein the oligonucleotide probe sets hybridize in a base-specific manner to their respective reference target nucleic acid molecule or reference target mRNA molecules, if present in the reference sample, and ligate to one another to form a secondary ligation product containing (a) the one or more detection oligonucleotide probe-specific portions or their complements, (b) the reference sample-specific portions and (c) the addressable array-specific portion or its complement, with the secondary ligation product for each secondary oligonucleotide probe set being distinguishable from other nucleic acid molecules or mRNA molecules in the secondary ligase detection reaction mixture, and, wherein the secondary oligonucleotide probe sets may hybridize to nucleic acid molecules or mRNA molecules in the reference sample other than their respective reference target nucleic acid molecules or reference target mRNA molecules but do not ligate together due to a presence of one or more mismatches and individually separate during the denaturation treatment;
blending the primary and secondary ligase detection reaction mixtures after subjecting them to one or more ligase detection reaction cycles and before said capturing, whereby the blended first and second ligase detection reaction mixtures are subjected to said capturing, said contacting, and said detecting; and
comparing relative amounts of the reporter labels on the primary and secondary ligation products, to provide a quantitative measure of the relative level of the one or more target nucleic acid molecules or target mRNA molecules in the test sample compared with that of the reference target nucleic acid molecules or reference target mRNA molecules in the reference sample as a result of each different labeled ligation product having a unique encryption code.

73. The method according to claim 72, wherein gene copy number is quantified.

74. The method according to claim 72, wherein mRNA copy number is quantified.

75. The method according to claim 72, wherein mRNA splice variant copy number is quantified.

76. The method according to claim 72, wherein one or more target nucleic acid molecules, differing by one or more single-base changes, insertions, deletions, or translocations, in a plurality of nucleic acid molecules are identified.

77. The method according to claim 71, wherein one or more target mRNA molecules differing by one or more splice site variations in a plurality of mRNA molecules are identified.

78. The method according to claim 71, wherein the detection oligonucleotide probes are in a form of one or more of detection oligonucleotide probe sets, each set characterized by (a) a first oligonucleotide probe and (b) a second oligonucleotide probe, wherein the detection oligonucleotide probes in a particular set are suitable for ligation together when hybridized to the primary ligation product sequence with corresponding detection oligonucleotide specific portions, but have a mismatch which interferes with such ligation when hybridized to any other nucleic acid molecule present and such that each detection oligonucleotide probe set contains a unique combination of detection oligonucleotide probes, and wherein said contacting comprises:
blending the captured primary ligation products, the one or more detection oligonucleotide probe sets, and the ligase to form a tertiary ligase detection reaction mixture and
subjecting the tertiary ligase detection reaction mixture to one or more ligase detection reaction cycles comprising a denaturation treatment, wherein any hybridized oligonucleotides are separated from the captured primary ligation products, and a hybridization treatment, wherein the one or more detection oligonucleotide probe sets hybridize in a base-specific manner to their respective ligation products, if present, and ligate to one another to form a tertiary ligation complex containing the one or more detection oligonucleotide probes or their complements hybridized to a nucleic acid molecule comprising the target-specific portions and the addressable array-specific portion or its complement, with the tertiary ligation complex for each one or more detection oligonucleotide probe set being distinguished from other nucleic acid molecules in the tertiary ligase detection reaction mixture by virtue of their containing a unique combination of detection oligonucleotide probes and addressable array-specific portions or their complements, and wherein the detection oligonucleotide probe sets may hybridize to nucleic acid molecules other than their respective captured primary ligation products but do not ligate together due to a presence of one or more mismatches and individually separate during the denaturation treatment.

79. The method according to claim 71 further comprising:
subjecting the primary ligation detection reaction mixture to exonuclease digestion after said subjecting the primary ligase detection reaction mixture to one or more ligase detection reaction cycles and prior to said contacting the primary ligation products, copies of primary ligation products, or complements thereof with the solid support under conditions effective to destroy unligated oligonucleotide probes.

80. The method according to claim 79, wherein for each primary oligonucleotide probe set, the first oligonucleotide probe contains a blocking group on its 5′ end, rendering the first oligonucleotide probe resistant to a 5′→3′exonuclease, and the second oligonucleotide probe contains a blocking group on its 3′ end, rendering it resistant to a 3′→5′exonuclease.

81. The method according to claim 71, wherein one of the oligonucleotide probes in the primary oligonucleotide probe set contains one or more detection oligonucleotide probe-specific portions or their complements.

82. The method according to claim 71, wherein both of the oligonucleotide probes in the primary oligonucleotide probe set contain one or more detection oligonucleotide probe-specific portions or their complements.

83. The method according to claim 71, wherein a complement of the primary ligation product is captured on the solid support.

84. The method according to claim 83 further comprising:
amplifying the primary ligation product prior to said contacting the primary ligation products, copies of primary ligation products, or complements thereof with the solid support.

85. The method according to claim 84, wherein said amplifying comprises:
hybridizing an oligonucleotide primer to the primary ligation product and
subjecting the hybridized oligonucleotide primer to a polymerase extension reaction under conditions effective to produce an extension product complementary to the primary ligation product.

86. The method according to claim 71, wherein the primary ligation product sequence is captured on the solid support.

87. The method according to claim 71, wherein the capture oligonucleotide probes at the particular sites on the solid support are the same as each other.

88. The method according to claim 71, wherein the capture oligonucleotide probes at the particular sites on the solid support are each unique with respect to one another.

89. The method according to claim 71, wherein the reporter labels are nanocrystals and said detecting comprises:
exciting the nanocrystals to produce emission spectra and
evaluating the emission spectra of the nanocrystals.

90. The method according to claim 71, wherein the ligase is selected from the group consisting of Thermus aquaticus ligase, Thermus thermophilus ligase, AK16D thermostable ligase, E. coli ligase, T4 ligase, and Pyrococcus ligase.

91. The method according to claim 71, wherein the target-specific portions of the oligonucleotide probes each have a hybridization temperature of 20-85° C.

92. The method according to claim 71, wherein the target-specific portions of the oligonucleotide probes are 15 to 30 nucleotides long.

93. The method according to claim 71, wherein the oligonucleotide probe sets are selected from the group consisting of ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, and mixtures thereof.

94. The method according to claim 71, wherein said method is used to detect infectious diseases caused by bacterial, viral, parasitic, and fungal infectious agents.

95. The method according to claim 94, wherein the infectious disease is caused by a bacterium selected from the group consisting of Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Actinomycetes.

96. The method according to claim 94, wherein the infectious disease is caused by a fungal infectious agent selected from the group consisting of Cryptococcus neoformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis.

97. The method according to claim 94, wherein the infectious disease is caused by a viral infectious agent selected from the group consisting of human immunodeficiency virus, human T-cell lymphocytotrophic virus, hepatitis viruses, Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

98. The method according to claim 94, wherein the infectious disease is caused by a parasitic infectious agent selected from the group consisting of Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

99. The method according to claim 71, wherein said method is used to detect genetic diseases.

100. The method according to claim 94, wherein the genetic disease is selected from the group consisting of 21 hydroxylase deficiency, cystic fibrosis, Fragile X Syndrome, Turner Syndrome, Duchenne Muscular Dystrophy, Down Syndrome, heart disease, single gene diseases, HLA typing, phenylketonuria, sickle cell anemia, Tay-Sachs Syndrome, thalassemia, Klinefelter's Syndrome, Huntington's Disease, autoimmune diseases, lipidosis, obesity defects, hemophilia, inborn errors in metabolism, and diabetes.

101. The method according to claim 71, wherein said method is used to detect cancer involving oncogenes, tumor suppressor genes, or genes involved in DNA amplification, replication, recombination, or repair, or SNPs or adjacent regions that serve as markers for copy number changes or loss of heterozygosity in such genes.

102. The method according to claim 101, wherein the cancer is associated with a gene selected from the group consisting of APC, AKT, ALT, AXL, BAX, Bcl2, Beta-Catenin, bFGF, BRCA1, BRCA2, Braf, Cdc25A, Cdk4, c-Fos, c-Jun, c-Kit, C-met, c-Myc, c-Ret, CSF1R, CSF2, c-Src, CYCD-CDK4, CYCE-CDK2, Cyclin D1, Cyclin E1, Cytokines, Dishevelled, E2F, E-Cadherin, EGFR, elF4E, ErbB-3, ErbB-4, FGFR-1, FGFR-2, FGFR-3, FGFR-4, FH4 (VEGFR-3), Fit-1 (VEGFR-1), Flk-I (VEGFR-2), Frizzled, G Proteins, GPCR, GRB2-SOS, GSK3 beta, Her2-neu, HGF, HSP27, HSP70, IFGII, IGFR1, K-ras, H-ras, N-ras, LT, MAPK, MDM2, MEK, MLH1, MSH2, MSH6, MYC, p15 p16^{Ink4a}, p19, p21^{Cip}, p27^{Kip}, p53, PDGFR alpha, PDGFR beta, PI3K, PP2A, PTEN, RAF, RAS, RB, Ron, RSK, RTK, Ski, Smad2, Smad4, ST, surviving, TbRII, TCF, Tcf4, TERT, TGF-Beta, TGF-Beta R, TIC2, TOR, VEGF, WAF1, Wisp-1, Wisp-3, WNT, or SNPs or adjacent regions that serve as markers for copy number changes or loss of heterozygosity in such genes, human papillomavirus Types 16 and 18, leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, ENT tumors, and loss of heterozygosity.

103. The method according to claim 71, wherein said method is used for environmental monitoring, forensics, and food and feed industry monitoring.

104. The method according to claim 71, wherein a plurality of primary oligonucleotide probe sets are utilized with each set characterized by (a) the first oligonucleotide probe being identical in each oligonucleotide probe set and (b) the second oligonucleotide probes in each set having a target-specific portion which is different in each second oligonucleotide probe at locations where single-base changes, insertions, deletions, or translocations occur.

105. The method according to claim 71, wherein the primary ligase detection reaction mixture is subjected to one ligase detection reaction cycle.

106. The method according to claim 71, wherein one or both of the oligonucleotide probes in the primary oligonucleotide probe set contain a plurality of detection oligonucleotide probe-specific portions or their complements.

107. The method according to claim 71, wherein, prior to said blending, said method further comprises:
providing a primer complementary to the target mRNA molecule;
providing a reverse transcriptase;
blending the primer, the reverse transcriptase, and the sample to form a reverse transcription mixture; and
subjecting the reverse transcription mixture to a reverse transcription reaction to produce cDNA copies of the target mRNA molecule.

108. The method according to claim 71 further comprising:
subjecting the sample to whole genome amplification prior to said blending.

109. The method according to claim 108, wherein said subjecting the sample to whole genome amplification comprises:
providing random primers;
providing a polymerase;
blending the sample, the random primers, and the polymerase to form a whole genome amplification reaction mixture; and
subjecting the whole genome amplification reaction mixture to a polymerase extension reaction under conditions effective to amplify the whole genome.

110. The method according to claim 71 further comprising:
providing a universal primer complementary to the primary ligation product;
providing a polymerase;
blending the captured ligation products, copies of the ligation products, or complements thereof, the universal primer, and the polymerase to form an isothermal amplification mixture prior to said contacting the captured ligation products, copies of the captured ligation products, or complements thereof, and the detection oligonucleotide probes; and
subjecting the isothermal amplification mixture to an isothermal amplification procedure to produce a complement of the primary ligation product.

111. The method according to claim 110, wherein the polymerase is Bst polymerase.

112. The method according to claim 110 further comprising:
subjecting the complement of the primary ligation product to restriction endonuclease digestion to cleave the complement of the primary ligation product.

113. The method according to claim 71, wherein the capture probe is provided with a hairpin oligonucleotide and, wherein said method further comprises:
ligating the hairpin oligonucleotide to the ligation products after said contacting the primary ligation products, copies of primary ligation products, or complements thereof with the solid support.

114. The method according to claim 71, wherein the target-specific portions of the primary oligonucleotide probe sets have a 3′ discriminating base.

115. The method according to claim 71, wherein within one or more of the ligase detection reaction cycles includes internal cycles comprising a hybridization treatment, wherein the oligonucleotide probe sets hybridize at adjacent positions in a base-specific manner to their respective target nucleic acid molecules, if present in the sample, and ligate to one another to form a ligation product a probe denaturation treatment, wherein, when the reaction mixture is heated to a temperature above that at which each target-specific portion melts, unligated probes separate from the nucleic acid molecules to which they are hybridized and when heated to a temperature below the melting temperature of each target-specific portion, ligation products hybridized to nucleic acid molecules accumulate with each successive internal cycle to provide a quantitative measure of the relative level of two or more target nucleic acid molecules in the sample.

116. The method according to claim 71, wherein the detection oligonucleotide probe-specific portions of the primary ligation products or complements thereof is selected from the set of sequences shown in FIGS. 114A, 115A, 116A, or 117A or their complements.

117. The method according to claim 71 further comprising:
ligating the detection oligonucleotide probes hybridized to a particular captured primary ligation product after said contacting the captured primary ligation products, copies of primary ligation products, or complements thereof and the detection oligonucleotide probes.

118. A method for identifying one or more target nucleic acid molecules, differing by one or more single-base changes, insertions, deletions, or translocations, in a plurality of nucleic acid molecules or identifying one or more target mRNA molecules differing by one or more splice site variations in a plurality of mRNA molecules, said method comprising:
providing a sample potentially containing one or more target nucleic acid molecules differing by one or more single-base changes, insertions, deletions, or translocations or one or more target mRNA molecules differing by one or more splice site variations;
capturing the target nucleic acid molecules or target mRNA molecules in the sample on one or a plurality of solid supports, so that they may be individually distinguished;
providing one or more primary oligonucleotide probe sets, each set characterized by (a) a first oligonucleotide probe, having a target-specific portion and a 5′ upstream portion complementary to one or more detection oligonucleotide probes and (b) a second oligonucleotide probe, having a target-specific portion and a 3′ downstream portion complementary to one or more detection oligonucleotide probes, wherein the oligonucleotide probes in a particular set are suitable for ligation together when hybridized to one another on a corresponding target nucleic molecule, but have a mismatch which interferes with such ligation when hybridized to any other nucleic molecule present in the sample, and such that each probe set contains a unique set of 5′ upstream and 3′ downstream portions;
providing a ligase;
blending the captured target nucleic acid molecules or target mRNA molecules, the one or more primary oligonucleotide probe sets, and the ligase to form a primary ligase detection reaction mixture;
subjecting the primary ligase detection reaction mixture to one or more ligase detection reaction cycles comprising a denaturation treatment, wherein any hybridized oligonucleotides are separated from the target nucleic acid molecule or target mRNA molecule, and a hybridization treatment, wherein the oligonucleotide probe sets hybridize in a base-specific manner to their respective target nucleic acid molecules or target mRNA molecule, if present in the sample, and ligate to one another to form a primary ligation product containing (a) the 5′ upstream portion, (b) the target-specific portions, and (c) the 3′ downstream portion with the primary ligation product remaining bound to the captured target nucleic acid molecule or target mRNA molecule, with the primary ligation product for each primary oligonucleotide probe set being distinguishable from other nucleic acids in the primary ligase detection reaction mixture by virtue of containing a unique set of 5′ upstream portion and 3′ downstream portion, and, wherein the oligonucleotide probe sets may hybridize to nucleic acid molecules in the sample other than their respective target nucleic acid molecule or target mRNA sequences but do not ligate together due to a presence of one or more mismatches and individually separate during the denaturation treatment;
providing detection oligonucleotide probes which bind to the 5′ upstream and 3′ downstream portions of the ligation products, wherein each detection oligonucleotide probe has a reporter label, such that each labeled primary ligation product has a unique detectable encryption code;
contacting the primary ligation products and the detection oligonucleotide probes under conditions effective to permit hybridization of the detection oligonucleotide probes to the primary ligation products so that a labeled captured primary ligation product is formed; and
detecting the reporter label on the primary ligation products, thereby indicating the presence of one or more target nucleic acid molecules differing by one or more single base changes, insertions, deletions, or translocations or one or more target mRNA molecules differing by one or more splice site variations are discriminated from one another during the ligase detection reaction and the discriminated target nucleic acid molecules or the target mRNA molecules are detected due to each different reporter labels having a unique encryption code with a different pattern of detectable emission spectra.

119. The method according to claim 118, wherein relative amounts of one or more of a plurality of target nucleic acid molecules in the test sample, differing by one or more single-base changes, insertions, deletions, or translocations or one or more target mRNA molecules differing by one or more splice site variables are quantified by comparison with a reference sample having reference target nucleic acid molecules or reference target mRNA molecule, said method comprising:
providing one or more secondary oligonucleotide probe sets, which differ from the primary oligonucleotide probe sets, wherein each of the secondary oligonucleotide probe sets are characterized by (a) a first oligonucleotide probe having a reference target-specific portion and a 5′ upstream portion complementary to one or more detection oligonucleotide probes and (b) a second oligonucleotide probe having a reference target-specific portion and a 3′ downstream portion complementary to one or more detection oligonucleotide probes, wherein the oligonucleotide probes in a particular secondary oligonucleotide probe set are suitable for ligation together when hybridized to one another on a corresponding reference target nucleic acid molecule or reference target mRNA molecule, but have a mismatch which interferes with such ligation when hybridized to any other nucleic acid or mRNA molecule present in the reference sample, and wherein one or both oligonucleotide probes in the secondary oligonucleotide probe set contain one or more detection oligonucleotide probe-specific portions or their complements such that each secondary oligonucleotide probe set contains a unique set of one or more detection oligonucleotide probe-specific portions or their complements;
blending the reference sample, the one or more secondary oligonucleotide probe sets, and the ligase to form a secondary ligase detection reaction mixture;
subjecting the secondary ligase detection reaction mixture to one or more ligase detection reaction cycles comprising a denaturation treatment, wherein any hybridized oligonucleotides are separated from reference target nucleic acid molecules or reference target mRNA molecules, and a hybridization treatment, wherein the oligonucleotide probe sets hybridize in a base-specific manner to their respective reference sample target nucleic acid molecule or reference target mRNA molecule, if present in the sample, and ligate to one another to form a secondary ligation product containing (a) the reference sample-specific portions and (b) the one or more detection oligonucleotide probe-specific portions or their complements with the secondary ligation product for each secondary oligonucleotide probe set being distinguishable from other nucleic acid molecules in the secondary ligase detection reaction mixture, and, wherein the secondary oligonucleotide probe sets may hybridize to nucleic acid molecules in the reference sample other than their respective reference target nucleic acid molecules or reference mRNA molecules but do not ligate together due to a presence of one or more mismatches and individually separate during the denaturation treatment;
blending the primary and secondary ligase detection reaction mixtures after subjecting them to one or more ligase detection reaction cycles and before said contacting, whereby the blended primary and secondary ligase detection reaction mixtures are subjected to said contacting and said detecting; and
comparing relative amounts of the reporter labels on the primary and secondary ligation products, to provide a quantitative measure of the relative level of the one or more target nucleic acid molecules or target mRNA molecules in the test sample compared with that of the reference target nucleic acid molecules or reference target mRNA molecules in the reference sample as a result of each different labeled ligation product having a unique encryption code.

120. The method according to claim 119, wherein gene copy number is quantified.

121. The method according to claim 119, wherein mRNA copy number is quantified.

122. The method according to claim 119, wherein mRNA splice variant copy number is quantified.

123. The method according to claim 118, wherein the detection oligonucleotide probes are in a form of one or more of detection oligonucleotide probe sets, each set characterized by (a) a first oligonucleotide probe and a second oligonucleotide probe, wherein the detection oligonucleotide probes in a particular set are suitable for ligation together when hybridized to the primary ligation products with corresponding detection oligonucleotide specific portions, but have a mismatch which interferes with such ligation when hybridized to any other nucleic acid molecule present, wherein each detection oligonucleotide probe set contains a unique combination of detection oligonucleotide probes, wherein said contacting comprises:
blending the primary ligation products, the one or more detection oligonucleotide probe sets, and the ligase to form a tertiary ligase detection reaction mixture and
subjecting the tertiary ligase detection reaction mixture to one or more ligase detection reaction cycles comprising a denaturation treatment, wherein any hybridized oligonucleotides are separated from the primary ligation products, and a hybridization treatment, wherein the one or more detection oligonucleotide probe sets hybridize in a base-specific manner to their respective primary ligation products, if present, and ligate to one another to form a tertiary ligation complex containing the one or more detection oligonucleotide probes or their complements hybridized to a nucleic acid molecule comprising the target-specific portions and the addressable array-specific portion or its complement, with the tertiary ligation complex for each one or more detection oligonucleotide probe set being distinguished from other nucleic acid molecules in the tertiary ligase detection reaction mixture by virtue of their containing a unique combination of detection oligonucleotide probes and addressable array-specific portions or their complements, and wherein the detection oligonucleotide probe sets may hybridize to nucleic acid molecules other than their respective primary ligation products but do not ligate together due to a presence of one or more mismatches and individually separate during the denaturation treatment.

124. The method according to claim 118, wherein the first oligonucleotide probe has a binding agent which is incorporated in any primary ligation product, said method further comprising:
providing the solid support with one or more attached binding partners to the binding agent;
contacting the primary ligation product, with the solid support under conditions effective for the binding agent and its binding partner to become coupled together, thereby immobilizing any primary ligation product to the solid support, wherein said detecting involves detecting the reporter label of the primary ligation product immobilized to the solid support.

125. The method according to claim 124, wherein the binding agent-binding partner pairs are selected from the group consisting of antibody-antigen binding partners, streptavidin-biotin binding partners, complementary oligonucleotides, amino group and EDC activated carboxylic acid group, thiol based binding partners, histidine moieties and nickel-NTA, and other chemical moieties that may be covalently or ionically linked to each other.

126. The method according to claim 118, wherein the reporter labels are nanocrystals and said detecting comprises:
exciting the nanocrystals to produce emission spectra and
evaluating the emission spectra of the nanocrystals.

127. The method according to claim 118, wherein the ligase is selected from the group consisting of Thermus aquaticus ligase, Thermus thermophilus ligase, AK16D thermostable ligase, E. coli ligase, T4 ligase, and Pyrococcus ligase.

128. The method according to claim 118, wherein the target-specific portions of the oligonucleotide probes each have a hybridization temperature of 20-85° C.

129. The method according to claim 118, wherein the target-specific portions of the oligonucleotide probes are 15 to 30 nucleotides long.

130. The method according to claim 118, wherein the oligonucleotide probe sets are selected from the group consisting of ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, and mixtures thereof.

131. The method according to claim 118, wherein said method is used to detect infectious diseases caused by bacterial, viral, parasitic, and fungal infectious agents.

132. The method according to claim 131, wherein the infectious disease is caused by a bacterium selected from the group consisting of Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Actinomycetes.

133. The method according to claim 131, wherein the infectious disease is caused by a fungal infectious agent selected from the group consisting of Cryptococcus neoformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis.

134. The method according to claim 131, wherein the infectious disease is caused by a viral infectious agent selected from the group consisting of human immunodeficiency virus, human T-cell lymphocytotrophic virus, hepatitis viruses, Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

135. The method according to claim 131, wherein the infectious disease is caused by a parasitic infectious agent selected from the group consisting of Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

136. The method according to claim 118, wherein said method is used to detect genetic diseases.

137. The method according to claim 136, wherein the genetic disease is selected from the group consisting of 21 hydroxylase deficiency, cystic fibrosis, Fragile X Syndrome, Turner Syndrome, Duchenne Muscular Dystrophy, Down Syndrome, heart disease, single gene diseases, HLA typing, phenylketonuria, sickle cell anemia, Tay-Sachs Syndrome, thalassemia, Klinefelter's Syndrome, Huntington's Disease, autoimmune diseases, lipidosis, obesity defects, hemophilia, inborn errors in metabolism, and diabetes.

138. The method according to claim 118, wherein said method is used to detect cancer involving oncogenes, tumor suppressor genes, or genes involved in DNA amplification, replication, recombination, or repair, or SNPs or adjacent regions that serve as markers for copy number changes or loss of heterozygosity in such genes.

139. The method according to claim 138, wherein the cancer is associated with a gene selected from the group consisting of APC, AKT, ALT, AXL, BAX, Bcl2, Beta-Catenin, bFGF, BRCA1, BRCA2, Braf, Cdc25A, Cdk4, c-Fos, c-Jun, c-Kit, C-met, c-Myc, c-Ret, CSF1R, CSF2, c-Src, CYCD-CDK4, CYCE-CDK2, Cyclin D1, Cyclin E1, Cytokines, Dishevelled, E2F, E-Cadherin, EGFR, elF4E, ErbB-3, ErbB-4, FGFR-1, FGFR-2, FGFR-3, FGFR-4, FH4 (VEGFR-3), Fit-1 (VEGFR-1), Flk-1 (VEGFR-2), Frizzled, G Proteins, GPCR, GRB2-SOS, GSK3 beta, Her2-neu, HGF, HSP27, HSP70, IFGII, IGFR1, K-ras, H-ras, N-ras, LT, MAPK, MDM2, MEK, MLH1, MSH2, MSH6, MYC, p15^{INK4b}, p16^{Ink4a}, p19^{ARF}, p21^{Cip}, p27^{Kip}, p53, PDGFR alpha, PDGFR beta, PI3K, PP2A, PTEN, RAF, RAS, RB, Ron, RSK, RTK, Ski, Smad2, Smad4, ST, surviving, TbRII, TCF, Tcf4, TERT, TGF-Beta, TGF-Beta R, TIC2, TOR, VEGF, WAF1, Wisp-1, Wisp-3, WNT, or SNPs or adjacent regions that serve as markers for copy number changes or loss of heterozygosity in such genes, human papillomavirus Types 16 and 18, leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, ENT tumors, and loss of heterozygosity.

140. The method according to claim 118, wherein said method is used for environmental monitoring, forensics, and food and feed industry monitoring.

141. The method according to claim 118, wherein a plurality of primary oligonucleotide probe sets are utilized with each set characterized by (a) the first oligonucleotide probe being identical in each oligonucleotide probe set and (b) the second oligonucleotide probes in each set having a target-specific portion which is different in each second oligonucleotide probe at locations where single-base changes, insertions, deletions, or translocations occur.

142. The method according to claim 118 wherein one or more target nucleic acid molecules, differing by one or more single-base changes, insertions, deletions, or translocations, in a plurality of nucleic acid molecules are identified.

143. The method according to claim 118, wherein one or more target mRNA molecules differing by one or more splice site variations in a plurality of mRNA molecules are identified.

144. The method according to claim 142, wherein the sample is mRNA, said method further comprising:
providing a primer complementary to the target mRNA molecule;
providing a reverse transcriptase;
blending the primer, the reverse transcriptase, and the sample to form a reverse transcription mixture; and
subjecting the reverse transcription mixture to a reverse transcription reaction to produce cDNA copies of the target mRNA molecule.

145. The method according to claim 144, wherein the primer is complementary to the target mRNA molecule.

146. The method according to claim 143, wherein the primer is a complementary to an oligodT added to the target mRNA molecule.

147. The method according to claim 118 further comprising:
subjecting the sample to nucleic acid fragmentation prior to capturing.

148. The method according to claim 118, wherein the primary ligase detection reaction mixture is subjected to one ligase detection reaction cycle.

149. The method according to claim 118, wherein one or both of the oligonucleotide probes in the primary oligonucleotide probe set contain a plurality of detection oligonucleotide probe-specific portions or their complements.

150. The method according to claim 118, wherein the target-specific portions of the primary oligonucleotide probe sets have a 3′ discriminating base.

151. The method according to claim 118, wherein the solid support is a paramagnetic bead and said method further comprises:
recovering the paramagnetic beads by magnetic attraction after said capturing and
placing the recovered paramagnetic beads on a slide.

152. The method according to claim 118, wherein within one or more of the ligase detection reaction cycles includes internal cycles comprising a hybridization treatment, wherein the oligonucleotide probe sets hybridize at adjacent positions in a base-specific manner to their respective target nucleic acid molecules, if present in the sample, and ligate to one another to form a ligation product a probe denaturation treatment, wherein, when the reaction mixture is heated to a temperature above that at which each target-specific portion melts, unligated probes separate from the nucleic acid molecules to which they are hybridized and when heated to a temperature below the melting temperature of each target-specific portion, ligation products hybridized to nucleic acid molecules accumulate with each successive internal cycle to provide a quantitative measure of the relative level of two or more target nucleic acid molecules in the sample.

153. The method according to claim 118, wherein the detection oligonucleotide probe-specific portions of the primary ligation products or complements thereof is selected from the set of sequences shown in FIGS. 108A, 109A, 110A, or 111A or their complements.

154. The method according to claim 118 further comprising:
ligating the detection oligonucleotide probes hybridized to the primary ligation product after said contacting the primary ligation products and the detection oligonucleotide probes.

155. The method according to claim 118 further comprising:
providing a linker containing a capture group;
blending the sample, the linker, a restriction endonuclease, and a ligase to form a ligase/restriction reaction mixture;
incubating the ligase/restriction reaction mixture under conditions to permit cleavage of DNA in the sample and ligation of linkers containing capture groups onto ends of resulting fragments.

156. The method according to claim 155 further comprising:
removing unligated linkers from the ligase/restriction reaction mixture after said incubating by filtration.

157. The method according to claim 118 further comprising:
providing a nucleotide containing a capture group;
providing a polymerase;
providing a restriction endonuclease which generates 5′ overhangs;
blending the sample, the nucleotide containing a capture group, the restriction endonuclease, and the polymerase to form a polymerase/restriction reaction mixture; and
incubating the polymerase/restriction reaction mixture under conditions to permit cleavage of DNA in the sample and extension with the nucleotide containing a capture group onto ends of resulting fragments.

158. A method for identifying one or more target nucleic acid molecules differing by one or more single-base changes, insertions, deletions, or translocations in a plurality of nucleic acid molecules comprising:
providing a test sample potentially containing one or more target nucleic acid molecules;
providing one or more primary oligonucleotide probe sets, each set characterized by (a) a first oligonucleotide probe, having a target-specific portion and a 5′ upstream portion containing one or more translational oligonucleotide probes or their complements, and (b) a second oligonucleotide probe, having a target-specific portion, wherein the oligonucleotide probes in a particular primary oligonucleotide set are suitable for ligation together when hybridized to a corresponding target nucleic acid molecule, but have a mismatch which interferes with such ligation when hybridized to any other nucleic acid molecule present in the sample, and such that each probe set contains a unique combination of translational oligonucleotide probes or their complements;
providing a ligase;
blending the sample, the one or more primary oligonucleotide probe sets, and the ligase to form a primary ligase detection reaction mixture;
subjecting the primary ligase detection reaction mixture to one or more ligase detection reaction cycles comprising a denaturation treatment, wherein any hybridized oligonucleotides are separated from the target nucleic acid molecules, and a hybridization treatment, wherein the oligonucleotide probes or the primary oligonucleotide probe sets hybridize in a base-specific manner to their respective target nucleic acid molecules, if present in the sample, and ligate to one another to form a primary ligation product containing (a) the translational oligonucleotide-specific portion or their complements and (b) the target-specific portions, with the ligation product for each primary oligonculeotide probe set being distinguishable from other nucleic acids in the primary ligase detection reaction mixture by virtue of containing a unique combination of translational oligonucleotide portions or their complements, and, wherein the primary oligonucleotide probe sets may hybridize to nucleic acid molecules in the sample other than their respective target nucleic acid molecules but do not ligate together due to a presence of one or more mismatches and individually separate during the denaturation treatment;
providing one or more secondary oligonucleotide probe sets, each set characterized by (a) a first oligonucleotide probe, having a translational oligonucleotide-specific portion and a 5′ upstream portion complementary to one or more detection oligonucleotide probe sequences and (b) a second oligonucleotide probe, having a translational oligonucleotide-specific portion, and a 3′ downstream portion complementary to one or more detection oligonucleotide probe sequences, wherein the oligonucleotide probes in a particular secondary oligonucleotide probe set are suitable for ligation together when hybridized to a corresponding complement of a primary ligation product, but have a mismatch which interferes with such ligation when hybridized to any other nucleic acid molecule present in the sample, and such that each secondary oligonucleotide probe set contains a unique set of 5′ upstream and 3′ downstream portions, blending the primary ligation products, the plurality of secondary oligonucleotide probe sets, and the ligase to form a secondary ligase detection reaction mixture;
subjecting the secondary ligase detection reaction mixture to one ligase detection reaction cycle comprising a denaturation treatment, wherein any hybridized oligonucleotide probes are separated from nucleic acid molecules to which they are hybridized, and a hybridization treatment, wherein the secondary oligonucleotide probe sets hybridize in a base-specific manner to their corresponding primary ligation products, if present, and ligate to one another to form a secondary ligation product containing (a) the 5′ upstream portion complementary to one or more distinct oligonucleotide probe sequences, (b) the upstream translational oligonucleotide-specific portion, (c) the downstream translational oligonucleotide-specific portion, and (d) the 3′ downstream portion complementary to one or more distinct oligonucleotide probe sequences, wherein the secondary oligonucleotide probe sets may hybridize to nucleic acid molecules other than their respective primary ligation products but do not ligate together due to a presence of one or more mismatches and individually separate during the denaturation treatment;
providing detection oligonucleotide probes which bind to the 5′ upstream portion and the 3′ downstream portion, wherein each detection oligonucleotide probe has a reporter label, such that each of the products has a unique detectable encryption code;
contacting the secondary ligation products and the detection oligonucleotide probes under conditions effective to permit hybridization of the detection oligonucleotide probes to the ligation products so that labeled, secondary ligation products are formed; and
detecting the reporter labels on the labeled, secondary ligation products, thereby indicating a presence of one or more target nucleic acid molecule in the sample, wherein sequences differing by one or more single-base changes, insertions, deletions, or translocations are discriminated from one another during the primary ligase detection reaction and the discriminated sequences are detected as a result of each different labeled secondary ligation product having a unique encryption code with a different pattern of detectable emission spectra.

159. The method according to claim 158, wherein relative amounts of one or more of a plurality of target nucleic acid molecules in the test sample, differing by one or more single-base changes, insertions, deletions, or translocations, are quantified by comparison with a reference sample having reference target nucleic acid molecules, said method comprising:
providing one or more reference oligonucleotide probe sets, which differ from the primary oligonucleotide probe sets, wherein each of the reference oligonucleotide probe sets are characterized by (a) a first oligonucleotide probe having a reference target-specific portion and a 5′ upstream portion containing one or more translational oligonucleotide probes or their complements and (b) a second oligonucleotide probe having a reference target-specific portion, wherein the oligonucleotide probes in a particular reference oligonucleotide probe set are suitable for ligation together when hybridized to one another on a corresponding reference target nucleic acid molecule, but have a mismatch which interferes with such ligation when hybridized to any other nucleic acid molecule present in the reference sample;
blending the reference sample, the one or more reference oligonucleotide probe sets, and the ligase to form a reference ligase detection reaction mixture;
subjecting the reference ligase detection reaction mixture to one or more ligase detection reaction cycles comprising a denaturation treatment, wherein any hybridized oligonucleotides are separated from reference target nucleic acid molecules, and a hybridization treatment, wherein the reference oligonucleotide probe sets hybridize in a base-specific manner to their respective reference sample target nucleic acid molecule, if present in the sample, and ligate to one another to form a reference ligation product sequence containing (a) the reference sample-specific portions and (b) the 5′ upstream portion containing one or more translation oligonucleotide probes or their complements with the reference ligation product for each reference oligonucleotide probe set being distinguishable from other nucleic acid molecules in the reference ligase detection reaction mixture, and, wherein the reference oligonucleotide probe sets may hybridize to nucleic acid molecules in the reference sample other than their respective reference target nucleic acid molecules but do not ligate together due to a presence of one or more mismatches and individually separate during the denaturation treatment;
blending the primary and reference ligase detection reaction mixtures after subjecting them to one or more ligase detection reaction cycles and before said blending the primary ligation products, the plurality of secondary oligonucleotide probe sets, and the ligase, said subjecting the secondary ligase detection reaction mixture to one or more ligase detection reaction cycles, said contacting the secondary ligation products and the detection oligonucleotide probes, whereby the blended primary and reference ligation products are subjected to said detecting; and
comparing relative amounts of the reporter labels on the secondary and reference ligation products, to provide a quantitative measure of the relative level of the one or more target nucleic acid molecules in the test sample compared with the reference sample as a result of each different labeled ligation product having a unique encryption code.

160. The method according to claim 158, wherein the detection oligonucleotide probes are in a form of one or more of detection oligonucleotide probe sets, each set characterized by (a) a first oligonucleotide probe and (b) a second oligonucleotide probe, wherein the detection oligonucleotide probes in a particular set are suitable for ligation together when hybridized to the secondary ligation product with corresponding detection oligonucleotide specific portions, but have a mismatch which interferes with such ligation when hybridized to any other nucleic acid molecule present and such that each detection oligonucleotide probe set contains a unique combination of detection oligonucleotide probes, wherein said contacting the secondary ligation products and the detection oligonucleotide probes comprises:
blending the secondary ligation products, the one or more detection oligonucleotide probe sets, and the ligase to form a tertiary ligase detection reaction mixture and
subjecting the tertiary ligase detection reaction mixture to one or more ligase detection reaction cycles comprising a denaturation treatment, wherein any hybridized oligonucleotides are separated from the secondary ligation products, and a hybridization treatment, wherein the one or more detection oligonucleotide probe sets hybridize in a base-specific manner to their respective secondary ligation products, if present, and ligate to one another to form a tertiary ligation complex containing the one or more detection oligonucleotide probes or their complements hybridized to the corresponding secondary ligation product comprising the 5′ upstream portion, the upstream translational oligonucleotide-specific portion, the downstream translational oligonucleotide-specific portion, and the 3′ downstream portion, with the tertiary ligation complex for each one or more detection oligonucleotide probe set being distinguished from other nucleic acid molecules in the tertiary ligase detection reaction mixture by virtue of their containing a unique combination of detection oligonucleotide probes, and wherein the detection oligonucleotide probe sets may hybridize to nucleic acid molecules other than their respective secondary ligation products but do not ligate together due to a presence of one or more mismatches and individually separate during the denaturation treatment.

161. The method according to claim 158, wherein one of the secondary oligonucleotide probes in the secondary oligonucleotide probe set contains one or more detection oligonucleotide probe-specific portions or their complements.

162. The method according to claim 158, wherein both of the oligonucleotide probes in the secondary oligonucleotide probe set contain one or more detection oligonucleotide probe-specific portions or their complements.

163. The method according to claim 158, wherein the reporter labels are nanocrystals and said detecting comprises:
exciting the nanocrystals to produce emission spectra and evaluating the emission spectra of the nanocrystals.

164. The method according to claim 158, wherein the ligase is selected from the group consisting of Thermus aquaticus ligase, Thermus thermophilus ligase, AK16D thermostable ligase, E. coli ligase, T4 ligase, and Pyrococcus ligase.

165. The method according to claim 158, wherein the target-specific portions of the oligonucleotide probes each have a hybridization temperature of 20-85° C.

166. The method according to claim 158, wherein the target-specific portions of the oligonucleotide probes are 15 to 30 nucleotides long.

167. The method according to claim 158, wherein the oligonucleotide probe sets are selected from the group consisting of ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, and mixtures thereof.

168. The method according to claim 158, wherein said method is used to detect infectious diseases caused by bacterial, viral, parasitic, and fungal infectious agents.

169. The method according to claim 168, wherein the infectious disease is caused by a bacterium selected from the group consisting of Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Actinomycetes.

170. The method according to claim 168, wherein the infectious disease is caused by a fungal infectious agent selected from the group consisting of Cryptococcus neoformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis.

171. The method according to claim 168, wherein the infectious disease is caused by a viral infectious agent selected from the group consisting of human immunodeficiency virus, human T-cell lymphocytotrophic virus, hepatitis viruses, Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

172. The method according to claim 168, wherein the infectious disease is caused by a parasitic infectious agent selected from the group consisting of Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

173. The method according to claim 158, wherein said method is used to detect genetic diseases.

174. The method according to claim 173, wherein the genetic disease is selected from the group consisting of 21 hydroxylase deficiency, cystic fibrosis, Fragile X Syndrome, Turner Syndrome, Duchenne Muscular Dystrophy, Down Syndrome, heart disease, single gene diseases, HLA typing, phenylketonuria, sickle cell anemia, Tay-Sachs Syndrome, thalassemia, Klinefelter's Syndrome, Huntington's Disease, autoimmune diseases, lipidosis, obesity defects, hemophilia, inborn errors in metabolism, and diabetes.

175. The method according to claim 158, wherein said method is used to detect cancer involving oncogenes, tumor suppressor genes, or genes involved in DNA amplification, replication, recombination, or repair, or SNPs or adjacent regions that serve as markers for copy number changes or loss of heterozygosity in such genes.

176. The method according to claim 175, wherein the cancer is associated with a gene selected from the group consisting of APC, AKT, ALT, AXL, BAX, Bcl2, Beta-Catenin, bFGF, BRCA1, BRCA2, Braf, Cdc25A, Cdk4, c-Fos, c-Jun, c-Kit, C-met, c-Myc, c-Ret, CSF1R, CSF2, c-Src, CYCD-CDK4, CYCE-CDK2, Cyclin D1, Cyclin E1, Cytokines, Dishevelled, E2F, E-Cadherin, EGFR, elF4E, ErbB-3, ErbB-4, FGFR-1, FGFR-2, FGFR-3, FGFR-4, FH4 (VEGFR-3), Fit-1 (VEGFR-1), Flk-1 (VEGFR-2), Frizzled, G Proteins, GPCR, GRB2-SOS, GSK3 beta, Her2-neu, HGF, HSP27, HSP70, IFGII, IGFR1, K-ras, H-ras, N-ras, LT, MAPK, MDM2, MEK, MLH1, MSH2, MSH6, MYC, p15^{INK4b}, p16^{Ink4a}, p19^{ARF}, p21^{Cip}, p27^{Kip}, p53, PDGFR alpha, PDGFR beta, PI3K, PP2A, PTEN, RAF, RAS, RB, Ron, RSK, RTK, Ski, Smad2, Smad4, ST, surviving, TbRII, TCF, Tcf4, TERT, TGF-Beta, TGF-Beta R, TIC2, TOR, VEGF, WAF1, Wisp-1, Wisp-3, WNT, or SNPs or adjacent regions that serve as markers for copy number changes or loss of heterozygosity in such genes, human papillomavirus Types 16 and 18, leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, ENT tumors, and loss of heterozygosity.

177. The method according to claim 158, wherein said method is used for environmental monitoring, forensics, and food and feed industry monitoring.

178. The method according to claim 158 further comprising:
providing a plurality of solid supports;
contacting the primary ligation products, copies of the primary ligation products, or complements thereof with the plurality of solid supports, prior to blending the primary ligation products, the plurality of oligonucleotide probe sets, and the ligase, under conditions effective to immobilize the primary ligation products, copies of the primary ligation products, or complements thereof to the solid supports; and
producing complements of the immobilized primary ligation products.

179. The method according to claim 178, wherein said producing complements comprises:
providing a universal primer complementary to the immobilized primary ligation product;
providing a polymerase;
blending the immobilized primary ligation product, the universal primer, and the polymerase to form an isothermal amplification mixture; and
subjecting the isothermal amplification mixture to an isothermal amplification procedure to produce a complement of the primary ligation product.

180. The method according to claim 179, wherein the polymerase is Bst polymerase.

181. The method according to claim 179 further comprising:
subjecting the complement of the primary ligation product to restriction endonuclease digestion to cleave the complement of the primary ligation product.

182. The method according to claim 178, wherein the second oligonucleotide probe of the first oligonucleotide probe set has a binding agent which is incorporated in any primary ligation product, and the solid support has one or more attached binding partner to the binding agent, whereby said contacting the primary ligation products, copies of the primary ligation products, or complements thereof with the plurality of solid supports is carried out under conditions effective for the binding agent and its binding partner to become coupled together, thereby immobilizing the primary ligation products to the solid support.

183. The method according to claim 182, wherein the binding agent-binding partner are selected from the group consisting of antibody-antigen binding partners, streptavidin-biotin binding partners, complementary oligonucleotides, amino group and EDC activated carboxylic acid group, thiol based binding partners, histidine moieties and nickel-NTA, and other chemical moieties that may be covalently or ionically linked to each other.

184. The method according to claim 178, wherein the solid support is a paramagnetic bead and said method further comprises:
recovering the paramagnetic beads by magnetic attraction after said contacting the primary ligation products, copies of the primary ligation products, or complements thereof with the plurality of solid supports and
placing the recovered paramagnetic beads on a microscope slide.

185. The method according to claim 158, wherein the second oligonucleotide probe of the primary oligonucleotide probe set has an addressable array-specific portion or its complement, said method further comprising:
providing a solid support with capture oligonucleotide probes immobilized at different sites, wherein the capture oligonulceotide probes have nucleotide sequences complementary to the addressable array-specific portions or their complement and
contacting the primary ligation products, copies of the primary ligation products, or complements thereof with the solid support with capture oligonucleotides, prior to said blending the primary ligation products, the plurality of second oligonucleotide probe sets, and the ligase, under conditions effective to hybridize the primary ligation products, copies of primary ligation products, or complements thereof to the capture oligonucleotide probes in a base-specific manner, thereby capturing the primary ligation products, copies of the primary ligation products, or complements thereof on the solid support at the site with the complementary capture oligonucleotide.

186. The method according to claim 185, wherein each of the capture probes are provided with a hairpin oligonucleotide and said method further comprises:
ligating the hairpin oligonucleotide to the primary ligation products after said contacting the primary ligation products, copies of primary ligation products, or complements thereof with the solid support.

187. The method according to claim 158, wherein the primary ligase detection reaction mixture is subjected to one ligase detection reaction cycle.

188. The method according to claim 158, wherein the secondary ligase detection reaction mixture is subjected to one ligase detection reaction cycle.

189. The method according to claim 158, wherein a plurality of primary oligonucleotide probe sets are utilized with each set characterized by (a) the first oligonucleotide probes being identical in each oligonucleotide probe set and (b) the second oligonucleotide probes in each set having a target-specific portion which is different in each second oligonucleotide probe at a location where single-base changes, insertions, deletions, or translocations occur.

190. The method according to claim 158, wherein the relative amounts of two or of a plurality of nucleic acid molecules, differing by one or more single-base changes, insertions, deletions, or translocations and present in a sample in unknown amounts with a plurality of target nucleic acid molecules being quantified, said method further comprising;
quantifying the relative amount of the ligation products, after said detecting and
comparing the relative amounts of the ligation products to provide a quantitative measure of the relative amounts of two or a plurality of target nucleic acid molecules in the sample.

191. The method according to claim 158, wherein within one or more of the ligase detection reaction cycles includes internal cycles comprising a hybridization treatment, wherein the oligonucleotide probe sets hybridize at adjacent positions in a base-specific manner to their respective target nucleic acid molecules, if present in the sample, and ligate to one another to form a ligation product and a probe denaturation treatment, wherein, when the ligase detection reaction mixture is heated to a temperature above that at which each target-specific portion melts, so unligated probes separate from the nucleic acid molecules to which they are hybridized, and when heated to a temperature below the melting temperature of each target-specific portion, ligation products hybridized to nucleic acid molecules accumulate with each successive internal cycle to provide a quantitative measure of the relative level of two or more target nucleic acid molecules in the sample.

192. The method according to claim 158, wherein the test sample comprises nucleic acid molecules isolated from a tumor, and the reference sample comprises nucleic acid molecules isolated from normal tissue or matched blood, wherein the relative level of the one or more target nucleic acid molecules in the test sample compared with the reference sample provides a measure of allele imbalance in the tumor at the corresponding loci.

193. The method according to claim 159, wherein the target specific portions of the primary oligonucleotide probe sets have a 3′ discriminating base.

194. The method according to claim 158, wherein the detection oligonucleotide probe-specific portions of the primary ligation products or complements thereof is selected from the set of sequences shown in FIGS. 121A or 123 A.

195. The method according to claim 158 further comprising:
ligating the detection oligonucleotide probes hybridized to a particular secondary ligation product after said contacting the secondary ligation products, copies of secondary ligation products, or complements thereof and the detection oligonucleotide probes.

196. The method according to claim 158 further comprising:
separating any unligated oligonucleotide probes from the ligation products.

197. A method of generating a linearly amplified representation of a whole genome comprising:
providing genomic DNA molecules;
subjecting the genomic DNA molecules to enzymatic digestion with a first restriction endonuclease to produce a degenerate oligonucleotide fragment with a degenerate overhang, wherein said subjecting is carried out in the presence of a hairpin linker containing an overhang complementary to the degenerate overhang and a modification complementing the 5′ end of the degenerate oligonucleotide fragment within a second restriction site, wherein the modification blocks restriction endonuclease cleavage on the 5′ side, but not the 3′ side, of the degenerate oligonucleotide fragment, a ligase, and a first restriction endonuclease, under conditions effective to permit cleavage of genomic DNA molecules and ligation of the hairpin linker containing the second restriction site onto ends of the degenerate oligonucleotide fragments;
removing unligated linkers;
providing a second restriction endonuclease;
providing a processive DNA polymerase with strand-displacement activity;
blending the enzymatically digested genomic DNA molecules, the second restriction endonuclease, and the polymerase to form a representational genome amplification mixture, and
incubating the representational genome amplification mixture under conditions effective to permit the second restriction endonuclease to nick the hairpin DNA on its unmodified strand and the polymerase to extend the degenerate oligonucleotide fragments at their free 3′ ends, wherein, as the polymerase extends and displaces the pre-existing strand, it reforms the second restriction site allowing for repeated nicking/polymerase extension and linear amplification of a representation of the whole genome.

198. A method of designing a plurality of labeled detection oligonucleotide probes for use in combinations of one to four or more probes to identify or quantify complementary sequences which will hybridize with little mismatch, wherein the plurality labeled oligonucleotide probes have melting temperatures within a narrow range, said method comprising:
providing a first set of a plurality of tetramers of four nucleotides linked together, wherein (1) each tetramer within the set differs from all other tetramers in the set by at least two nucleotide bases, (2) no two tetramers within a set are complementary to one another, (3) no tetramers within a set are palindromic or dinucleotide repeats, and (4) no tetramer within a set has less than one or more than three G and C nucleotides;
linking groups of 2 to 4 tetramers from the first set together to form a collection of multimer units;
removing from the collection of multimer units all multimer units formed from the same tetramer and all multimer units having a melting temperature in ° C. of less than 6 times the number of tetramers forming a multimer unit, to form a modified collection of multimer units;
selecting from the modified collection of multimer units a second collection of multimer units such that no consecutive tetramer pair is used twice;
adding 1 or 2 tetramers to either or both ends of the second collection of multimers to generate a new set of modified multimer units with higher melting temperatures, such that no consecutive tetramer pair is used twice;
arranging the new set of modified multimer units in a list in order of melting temperature;
removing from the set of modified multimer units those units having a melting temperature in ° C. of less than 12 times the number of tetramers and more than 18 times the number of tetramers, to form a further collection of multimer units; and
linking reporter labels to the further collection of multimer units, to form labeled detection oligonucleotide probes.

199. The method according to claim 198, wherein the reporter labels are nanocrystals.

200. The method according to claim 198, wherein the further collection of multimer units is shown in FIG. 96.

201. The method according to claim 198, wherein the set of tetramers is shown in Table 1 and complements thereof.

202. The method according to claim 198, wherein the oligonucleotide probes are selected from the group consisting of ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, and mixtures thereof.

203. A method of designing a plurality of translational oligonucleotides for attachment to target-specific oligonucleotide probes to identify or quantify complementary sequences which will hybridize with little mismatch, wherein the plural translating oligonucleotide sequences have melting temperatures within a narrow range, said method comprising:
providing a first set of a plurality of tetramers of four nucleotides linked together, wherein (1) each tetramer within the set differs from all other tetramers in the set by at least two nucleotide bases, (2) no two tetramers within a set are complementary to one another, (3) no tetramers within a set are palindromic or dinucleotide repeats, and (4) no tetramer within a set has less than one or more than three G and C nucleotides;
linking groups of 2 to 4 tetramers from the first set together to form a collection of multimer units;
removing from the collection of multimer units all multimer units formed from the same tetramer and all multimer units having a melting temperature in ° C. of less than 3 times the number of tetramers forming a multimer unit, to form a modified collection of multimer units;
arranging the modified collection of multimer units in a list in order of melting temperature;
randomizing, in 0.1° C. increments of melting temperature, the order of the modified collection of multimer units;
dividing alternating multimer units in the list into first and second subcollections, each arranged in order of melting temperature;
inverting the order of the second subcollection;
linking in order the first subcollection of multimer units to the inverted second subcollection of multimer units in order to form a collection of double multimer units;
arranging the collection of double multimer units in a list in order of melting temperature;
removing from the ordered collection of double multimer units those units having a melting temperature in ° C. of less than 12 times the number of tetramers and more than 18 times the number of tetramers, to form a modified collection of multimer units; and
linking the double multimer units to a target-specific oligonucleotide probe.

204. The method according to claim 203, wherein the collection of double multimer units is shown in FIG. 118A.

205. The method according to claim 203, wherein the set of tetramers is shown in Table 1 and complements thereof.

206. The method according to claim 203, wherein the oligonucleotide probes are selected from the group consisting of ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, and mixtures thereof

207. A collection of labeled detection oligonucleotide probes comprising:
a collection of detection oligonucleotide probes to which complementary oligonucleotide probes will hybridize, within a narrow temperature range of greater than 24° C. with little mismatch, wherein the oligonucleotide probes are formed from sets of tetramers where (1) each tetramer within the set differs from all other tetramers in the set by at least two nucleotide bases, (2) no two tetramers within a set are complementary to one another, (3) no tetramers within a set are palindromic or dinucleotide repeats, and (4) no tetramer within a set has less than one or more than three G and C nucleotides, and wherein the collection of oligonucleotide probes has oligonucleotides having a melting temperature in ° C. less than 12 times the number of tetramers and more than 18 times the number of tetramers and
reporter labels linked to each of the oligonucleotide probes in the collection.

208. The collection according to claim 207, wherein the reporter labels are nanocrystals.

209. The collection according to claim 206, wherein the collection of detection oligonucleotide probes is shown in FIG. 119A.

210. The collection according to claim 207, wherein the set of tetramers is shown in Table 1 and complements thereof.

211. The method according to claim 207, wherein the oligonucleotide probes are selected from the group consisting of ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, and mixtures thereof.

212. A collection of fusion oligonucleotide probes comprising:
a collection of translational oligonucleotide probes to which complementary oligonucleotide probes will hybridize, within a narrow temperature range of greater than 24° C. with little mismatch, wherein the oligonucleotide probes are formed from sets of tetramers where (1) each tetramer within the set differs from all other tetramers in the set by at least two nucleotide bases, (2) no two tetramers within a set are complementary to one another, and (3) no tetramers within a set are palindromic or dinucleotide repeats, and (4) no tetramer within a set has less than one or more than three G and C nucleotides, and wherein the collection of oligonucleotide probes has oligonucleotides having a melting temperature in ° C. less than 12 times the number of tetramers and more than 18 times the number of tetramers removed and
target-specific oligonucleotide probes linked to each of the oligonucleotide probes in the collection.

213. The collection according to claim 212, wherein the collection of translation oligonucleotide probes is shown in FIGS. 121A and 123A.

214. The collection according to claim 212, wherein the set of tetramers is shown in Table 1 and complements thereof.

215. The method according to claim 212, wherein the oligonucleotide probes are selected from the group consisting of ribonucleotides, deoxyribonucleotides, modified ribonucleotides, modified deoxyribonucleotides, peptide nucleic acids, modified peptide nucleotide analogues, modified phosphate-sugar backbone oligonucleotides, nucleotide analogues, and mixtures thereof
